(19) United States
(12) Patent Application Publication
(10) Pub. No.: US 2024/0401061 A1
(43) Pub. Date: Dec. 5, 2024

(54) COMPOUNDS AND METHODS FOR REDUCING PLN EXPRESSION

(71) Applicant: Ionis Pharmaceuticals, Inc., Carlsbad, CA (US)

(72) Inventors: Dieter A. KUBLI, Santee, CA (US); Brooke A. ANDERSON, San Diego, CA (US); Adam MULLICK, Carlsbad, CA (US); Eric E. SWAYZE, Encinitas, CA (US)

(73) Assignee: Ionis Pharmaceuticals, Inc., Carlsbad, CA (US)

(21) Appl. No.: 18/626,889

(22) Filed: Apr. 4, 2024

Related U.S. Application Data
(60) Provisional application No. 63/561,700, filed on Mar. 5, 2024.
(60) Provisional application No. 63/508,453, filed on Jun. 15, 2023.
(60) Provisional application No. 63/494,415, filed on Apr. 5, 2023.

(51) Int. Cl. C12N 15/113; A61P 9/04

(52) U.S. Cl. CPC C12N 15/1138; A61P 9/04; C12N 2310/14; C12N 2310/313; C12N 2310/321; C12N 2310/3341; C12N 2310/3513

(57) ABSTRACT

Provided are oligomeric compounds, methods, and pharmaceutical compositions for reducing the amount or activity of PLN RNA in a cell or animal, and in certain instances reducing the amount of phospholamban protein in a cell or animal. Such oligomeric compounds, methods, and pharmaceutical compositions are useful to treat cardiomyopathy, heart failure, or arrhythmia.

### CROSS-REFERENCE TO RELATED PATENT APPLICATIONS

This specification claims the benefit of priority to U.S. Provisional Patent Application No. 63/494,415 (filed 5 Apr. 2023), U.S. Provisional Patent Application No. 63/508,453 (filed 15 Jun. 2023) and U.S. Provisional Patent Application No. 63/561,700 (filed 5 Mar. 2024). The entire text of the above-referenced patent applications is incorporated by reference into this specification.

### SEQUENCE LISTING

The present application is being filed concurrently with a Sequence Listing in electronic format. The Sequence Listing is provided as a file entitled 201366-WO-PCT.xml, created on Mar. 8, 2024, which is 1,520 KB in size. The contents of the electronic format of the sequence listing are incorporated herein by reference in their entirety.

### FIELD

The present invention relates to compounds, compositions, and uses therefor, including methods for decreasing the levels of PLN expression, PLN RNA, and/or the levels (and/or activity) of phospholamban protein, as well as to methods for preventing, treating, and/or ameliorating at least one symptom of, a cardiac disease, disorder or condition, such as, for example, cardiomyopathy, heart failure, or arrhythmia.

### BACKGROUND

Heart pumping action, which forces blood throughout the body, is generated by the repetitive contraction of cardiac muscle (i.e., myocardium). The rhythmic contraction process of cardiac muscle is regulated by changes in cytosolic calcium ion concentration in cardiac muscle cells (referred to as cardiomyocytes). Increases in cytosolic calcium ion concentration are associated with myocardial contraction (during systole), whereas decreases in cytosolic calcium ion concentration are associated with heart muscle relaxation (during diastole). The sarcoplasmic reticulum, a membranous intracellular tubular structure within cardiac and skeletal muscle cells, is responsible for contractile calcium handling (i.e., uptake and storage of calcium ions from the cytosol during relaxation and release of sequestered calcium ions into the cytosol for muscle contraction); and is associated with multiple proteins that facilitate and regulate its uptake and release of calcium ions. One such protein is sarco(endo)plasmic reticulum calcium ATPase (SERCA), which functions to pump cytosolic calcium into the sarcoplasmic reticulum during relaxation of cardiac muscle. SERCA is regulated by phospholamban (encoded by PLN), a phosphoprotein which inhibits SERCA activity in its dephosphorylated state by decreasing affinity of SERCA for calcium ions. This in turn attenuates filling the sarcoplasmic reticulum calcium store and diminishes contractile force development. Phospholamban phosphorylation reverses its inhibition of SERCA.

Heart failure, a condition wherein the heart is not able to provide sufficient blood flow to the body, is a leading cause of death worldwide. Heart failure can be chronic, developing gradually over time, or acute, which is a more sudden, rapid decline in heart functioning. Heart failure may be caused by conditions that damage, weaken and/or overwork the heart, including cardiomyopathy. Cardiomyopathy refers to heart muscle diseases, disorders and conditions that weaken the heart, eventually resulting in inability to pump effectively. As the heart weakens, normal heart muscle can thicken, stiffen, or thin out, impairing its ability to pump blood which can lead to heart failure.

Arrhythmia is an irregular or abnormal heartbeat and the leading cause of sudden cardiac deaths. Arrhythmia originating in the ventricles (lower chambers of the heart) is particularly dangerous and cause the heart to beat too fast, which impairs blood circulation and can result in cardiac arrest. Ventricular fibrillation (vfib) is a rapid uncoordinated heart rhythm in which the heart's electrical signals often lack a normal and repetitive pattern. Ventricular tachycardia (vtac) is a rapid heart rhythm, which if too fast, can prevent the heart from effectively beating or pumping blood to the entire body and cause loss of consciousness.

Effective management of intracellular calcium is a significant factor in regulation of contractile force in cardiac muscle. Symptoms of heart failure include reduced contractile function, which can appear as blunted, slowed, dysynchronous contraction, and impaired relaxation of the heart. Aberrant calcium cycling and defective intracellular calcium ion homeostasis within cardiac muscle cells are associated with contractile dysfunction and arrhythmias in failing myocardium. There remains a need for therapies targeting cardiac dysfunction in heart failure and conditions associated with heart failure. Therapeutics targeting cardiac calcium regulation may provide a new class of compounds for effective management of contractile function and therapy for subjects at risk of cardiomyopathy and heart failure.

### SUMMARY

Provided herein are compounds, pharmaceutical compositions, and methods of use for reducing the amount or activity of PLN RNA, and in certain embodiments reducing the expression of phospholamban in a cell or subject. In certain embodiments, the subject has a disease or disorder associated with cardiac calcium regulation, regulation of contractile force of cardiac muscle or a mutation in PLN. In certain embodiments, the subject has a cardiomyopathy. In certain embodiments, the subject has cardiac arrythmia. In certain embodiments, the subject has heart failure. In certain embodiments, compounds useful for reducing the amount or activity of PLN RNA are oligomeric compounds, oligomeric duplexes, antisense compounds, RNAi agents. In certain embodiments, compounds useful for decreasing expression of phospholamban are oligomeric compounds, oligomeric duplexes, antisense compounds, and RNAi agents.

Also provided are methods useful for ameliorating at least one symptom of a cardiac disorder or cardiac injury. In certain embodiments the disorder is cardiomyopathy. In certain embodiments, the disorder is arrythmia. In certain embodiments the injury is heart failure. In certain embodiments, the symptom is selected from pain, hypokalemia, heart palpitations (e.g., irregular tempo, fast heartbeat, forceful heartbeat, or fluttering), chest pain, fatigue, shortness of breath, weakness, lightheadedness, dizziness, fainting episode(s), nausea, confusion, intolerance to exertion, blood clots, or a combination thereof.

Provided are modified oligonucleotides and compounds and compositions comprising them, including, but not limited to, antisense compounds, oligomeric compounds, oligomeric duplexes and pharmaceutical compositions comprising modified oligonucleotides. In certain embodiments, a modified oligonucleotide provided herein comprises a nucleobase sequence at least 80% complementary to an equal length portion of a PLN nucleic acid. In certain embodiments, the modified oligonucleotide consists of 12 to 35, 14 to 30, 15 to 28, 16 to 25, or 18 to 23 linked nucleosides targeting PLN nucleic acid. In certain embodiments, a modified oligonucleotide provided herein comprises a sequence of nucleobases complementary to an equal length portion of the nucleobase sequence of SEQ ID NO:1 and/or SEQ ID NO:2.

In certain embodiments, a modified oligonucleotide provided herein comprises at least one modified sugar moiety and/or at least one modified internucleoside linkage. Modified oligonucleotides and compositions comprising them, including, but not limited to, oligomeric compounds, oligomeric duplexes, antisense compounds and pharmaceutical compositions, described herein are useful for reducing or inhibiting PLN expression in a cell, organ, tissue, system, organism or animal.

Additionally provided herein are methods for reducing or inhibiting PLN expression, PLN RNA levels and/or phospholamban protein levels and/or activity in a cell or organism, including, for example, an animal. In certain embodiments, the methods include contacting a cell or subject with a composition provided herein, comprising a modified oligonucleotide, oligomeric compound, and/or oligomeric duplex. In certain embodiments, the subject is a human who has or is at risk of having a disease, disorder, condition or injury associated with cardiac calcium regulation, regulation of contractile force of cardiac muscle or a mutation in PLN. In particular embodiments, the subject is a human who has or is at risk of having heart failure. In certain particular embodiments, the subject is a human who has or is at risk of having arrythmia and/or cardiomyopathy, including, for example, dilated, hypertrophic, arrhythmogenic or restrictive cardiomyopathy.

Also provided herein are methods of preventing, treating, slowing the development or progression of, or ameliorating a disease, disorder, condition or injury associated with cardiac calcium regulation, regulation of contractile force of cardiac muscle or a mutation in PLN. In certain embodiments, methods provided herein include methods of preventing, treating, slowing the development or progression of, or ameliorating cardiomyopathy, heart failure, and/or arrhythmia. In some embodiments, methods provided herein for preventing, treating, slowing the development or progression of, or ameliorating a disease, disorder, condition or injury associated with cardiac calcium regulation, regulation of contractile force of cardiac muscle or a mutation in PLN, e.g., cardiomyopathy, heart failure, and/or arrhythmia, comprise administering to a subject, e.g., a human subject, having or at risk of having a disease, disorder, condition or injury associated with cardiac calcium regulation, regulation of contractile force of cardiac muscle or a mutation in PLN, e.g., cardiomyopathy, heart failure, and/or arrhythmia, a composition provided herein, e.g., a modified oligonucleotide, oligomeric compound, oligomeric duplex or pharmaceutical composition provided herein. Also provided herein are methods of preventing, treating, ameliorating, delaying the onset of, or reducing frequency of at least one symptom of cardiomyopathy, arrhythmia, and/or heart failure. Symptoms of cardiomyopathy, arrhythmia and/or heart failure include, but are not limited to, shortness of breath, trouble breathing, fatigue, swelling in the ankles, legs or feet, cough, irregular heart beat, heart palpitations, dizziness, lightheadedness, syncope, weakness, reduced ability to exercise and abdominal bloating. In some embodiments, methods provided herein for preventing, treating, ameliorating, delaying the onset of, or reducing frequency of at least one symptom of cardiomyopathy, arrhythmia and/or heart failure include administering to a subject, e.g., a human subject, having or at risk of having the at least one symptom, a composition provided herein, e.g., a modified oligonucleotide, oligomeric compound, oligomeric duplex or pharmaceutical composition provided herein.

### BRIEF DESCRIPTION OF THE DRAWINGS

FIGS. 1A-1B depict experimental results from studies described in Example 6 showing potency of 5′-C16, 3′—C16, 5′—BCY17901, or 3′-BCY17901 conjugated modified oligonucleotides and oligomeric duplexes targeting human PLN.

FIGS. 2A-2B depict experimental results from studies described in Example 11 showing potency of BCY17901-conjugated modified oligomeric duplexes targeting human PLN.

FIG. 3 depicts experimental results from duration study described in Example 14 showing potency and duration of BCY17901-conjugated modified oligomeric duplexes targeting human PLN.

FIG. 4 depicts a model of PLN targeted compound activity improvements based on results described herein (see also Example 15).

### DETAILED DESCRIPTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

Section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

The following definitions are provided, along with additional definitions throughout the specification, for a complete understanding of the instant invention. Unless specific definitions are provided herein, nomenclature used in connection with, and procedures and techniques of, analytical chemistry, synthetic organic chemistry, and medicinal and pharmaceutical chemistry described herein are those well-known and commonly used in the art. Unless otherwise indicated, certain terms have the following meanings:

As used herein, “2′-deoxynucleoside” means a nucleoside comprising a 2′-deoxy sugar moiety. Unless otherwise indicated, a 2′-deoxynucleoside is a 2′-β-D-deoxynucleoside which comprises a 2′-β-D-deoxyribosyl sugar moiety, which has the β-D ribosyl configuration as found in naturally occurring deoxyribonucleic acid (DNA).

As used herein, “2′-deoxy sugar moiety” means a 2′-H(H) deoxyribosyl sugar moiety. Unless otherwise indicated, a 2′-deoxy sugar moiety is a 2′-β-D-deoxyribosyl sugar moiety, which has the β-D ribosyl configuration as found in naturally occurring deoxyribonucleic acids (DNA). Herein, in the context of an oligomeric compound comprising a ribonucleic acid oligonucleotide (e.g., an siRNA), a 2′-deoxy sugar moiety is considered e.g., a modified sugar moiety.

As used herein, “2′-MOE” means a 2′-OCH₂CH₂OCH₃ group in place of the 2′—OH group of a ribosyl sugar moiety. A “2′-MOE sugar moiety” means a sugar moiety with a 2′-OCH₂CH₂OCH₃ group in place of the 2′—OH group of a ribosyl sugar moiety. Unless otherwise indicated, a 2′-MOE sugar moiety is in the β-D-ribosyl configuration. “MOE” means O-methoxyethyl.

As used herein, “2′-MOE nucleoside” or “2′-O(CH₂)₂OCH₃ nucleoside” means a nucleoside comprising a 2′-MOE sugar moiety (or 2′-OCH₂CH₂OCH₃ ribosyl sugar moiety). As used herein, “2′-OMe” means a 2′-OCH₃ group in place of the 2′—OH group of a ribosyl sugar moiety. A “2′-OMe sugar moiety” or “2′-O-methyl sugar moiety” means a sugar moiety with a 2′-OCH₃ group in place of the 2′—OH group of a ribosyl sugar moiety. Unless otherwise indicated, a 2′-OMe has the 3-D-ribosyl stereochemical configuration.

As used herein, “2′-OMe nucleoside” means a nucleoside comprising a 2′-OMe sugar moiety.

As used herein, “2′-F” means a 2′-fluoro group in place of the 2′—OH group of a furanosyl sugar moiety. A “2′-F sugar moiety” (i.e., a “2′-fluoro sugar moiety”) means a sugar moiety with a 2′-F (i.e., a 2′-fluoro) group in place of the 2′—OH group of a furanosyl sugar moiety. Unless otherwise indicated, a 2′-F sugar moiety is in the β-D-ribosyl configuration.

As used herein, “2′-F nucleoside” means a nucleoside comprising a 2′-F sugar moiety.

As used herein, “2′-substituted nucleoside” means a nucleoside comprising a 2′-substituted furanosyl sugar moiety. As used herein, “2′-substituted” in reference to a sugar moiety means a sugar moiety wherein at least one 2′-substituent group is other than H. Examples of 2′-substituted modified sugar moieties include sugar moieties comprising a 2′-substituent group independently selected from 2′-F, 2′-MOE, 2′-OMe, and cEt.

As used herein, “5-methylcytosine” means a cytosine modified with a methyl group attached at the 5 position. A 5-methylcytosine is a modified nucleobase.

As used herein, “abasic sugar moiety” means a sugar moiety of a nucleoside that is not attached to a nucleobase. Such abasic sugar moieties are sometimes referred to in the art as “abasic nucleosides.”

As used herein, “ameliorate” means improvement in or lessening of at least one symptom of an associated disease, disorder or condition. In certain embodiments, amelioration is reduction in severity or frequency of a symptom or the delayed onset or slowing of progression in the severity or frequency of a symptom. Progression, frequency, or severity indicators may be determined by subjective or objective measures known in the art and/or described herein.

As used herein, “antisense activity” means any detectable and/or measurable change attributable (whether directly and/or indirectly) to hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound. In certain embodiments, compounds have antisense activity when they reduce or inhibit the amount or activity of a target nucleic acid by 25% or more in an in vitro assay. In certain embodiments, compounds have antisense activity when they reduce or inhibit the amount or activity of a target nucleic acid by 25% or more in an in vivo assay. In certain embodiments antisense activity is assessed in a standard assay.

As used herein, “antisense compound” means an antisense oligonucleotide and optionally one or more additional features, e.g., a paired oligonucleotide, a conjugate group and/or a terminal group. As used herein, “antisense oligonucleotide” means an oligonucleotide that is capable of hybridizing to a target nucleic acid and is capable of at least one antisense activity. In certain embodiments, antisense compounds selectively affect one or more target nucleic acid. In certain embodiments, an antisense compound is a modified oligonucleotide provided herein that is capable of hybridizing to a target nucleic acid and is capable of at least one antisense activity. An antisense oligonucleotide may be paired with a second oligonucleotide (herein, a “sense oligonucleotide”) that is complementary to the antisense oligonucleotide (that is capable of hybridizing to an antisense oligonucleotide to form a double-stranded antisense oligonucleotide, a duplex antisense oligonucleotide), may be an unpaired antisense oligonucleotide (a singled-stranded antisense oligonucleotide) or may be a “hairpin” oligonucleotide that has at least one region that is self-complementary. As used herein, “sense compound” means a sense oligonucleotide and optionally one or more additional features, e.g., a conjugate group.

As used herein, “bicyclic nucleoside” means a nucleoside comprising a bicyclic sugar moiety.

As used herein, “bicyclic sugar” or “bicyclic sugar moiety” means a modified sugar moiety comprising two rings, wherein the second ring is formed via a bridge connecting two of the atoms in the first ring thereby forming a bicyclic structure, wherein the first ring of the bicyclic sugar moiety is a furanosyl ring. Examples of bicyclic sugar moieties include LNA (locked nucleic acid) sugar moiety and cEt sugar moiety as defined herein.

As used herein, “blunt” or “blunt ended” in reference to an oligomeric duplex means that there are no terminal unpaired nucleotides (i.e., no overhanging nucleotides). One or both ends of an oligomeric duplex can be blunt.

As used herein, “cell-targeting moiety” means a conjugate group or portion of a conjugate group that is capable of binding to a particular cell type or particular cell types. In certain embodiments, a cell-targeting moiety binds to a surface moiety, such as a surface receptor on a particular cell type.

As used herein, “cleavable moiety” means a bond or group of atoms that is cleaved under physiological conditions, e.g., in a cell and/or upon administration to a subject.

As used herein, “complementary” in reference to an oligonucleotide or portion thereof means that at least 70% of the nucleobases of such oligonucleotide or portion thereof and the nucleobases of another nucleic acid or portion thereof are capable of hydrogen bonding with one another when the nucleobase sequence of the oligonucleotide and the other nucleic acid are aligned in opposing directions. As used herein, “complementary nucleobases” means nucleobases that are capable of forming hydrogen bonds with one another. Complementary nucleobase pairs include adenine (A) and thymine (T); adenine (A) and uracil (U); cytosine (C) and guanine (G); and 5-methylcytosine (“^{m}C) and guanine (G). Certain modified nucleobases that pair with unmodified nucleobases or with other modified nucleobases are known in the art. For example, hypoxanthine (I), the nucleobase of the nucleoside inosine, can pair with adenine, cytosine, thymine, or uracil. Herein, hypoxanthine (I) is considered a complementary nucleobase to thymine (T), adenine (A), uracil (U), and cytosine (C). Complementary oligonucleotides and/or nucleic acids need not have nucleobase complementarity at each nucleoside. Rather, some mismatches are tolerated. As used herein, “fully complementary” or “100% complementary” in reference to an oligonucleotide, or a portion thereof, means that the oligonucleotide, or portion thereof, is complementary to another oligonucleotide or nucleic acid at each nucleobase of the shorter of the two oligonucleotides, or at each nucleoside if the oligonucleotides are the same length.

As used herein, “complementary region” in reference to an oligonucleotide or portion thereof is a range of nucleobases of the oligonucleotide that is complementary to a nucleobase sequence of an equal-length region of a second oligonucleotide or region thereof (e.g., an oligonucleotide and a target nucleic acid, or an antisense oligonucleotide and a sense oligonucleotide), or to a nucleobase sequence of an equal-length region within a second region of the oligonucleotide (e.g., in a “hairpin oligonucleotide”). A complementary region of an oligonucleotide may be a portion of an oligonucleotide or may include the entire oligonucleotide or may include substantially all of the oligonucleotide.

As used herein, “constrained ethyl” or “cEt” or “cEt sugar moiety” means a β-D ribosyl bicyclic sugar moiety wherein the second ring of the bicyclic sugar is formed via a bridge connecting the 4′-carbon and the 2′-carbon of the β-D ribosyl sugar moiety, wherein the bridge has the formula 4′-CH(CH₃)—O-2′, and wherein the methyl group of the bridge is in the S configuration.

As used herein, “cEt nucleoside” means a nucleoside comprising a cEt sugar moiety.

As used herein, “hybridization” means the process of two complementary nucleic acid sequences (e.g., oligonucleotides, nucleic acids) annealing or bonding together to form a duplex or double stranded region or molecule. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid sequences in separate molecules include, but are not limited to, an antisense compound and a nucleic acid target. In certain embodiments, complementary nucleic acid sequences in separate molecules include, but are not limited to, an oligonucleotide of the invention and a nucleic acid target. In certain embodiments, complementary nucleic acid sequences in separate molecules include, but are not limited to, an antisense compound and a sense compound. In certain embodiments, complementary nucleic acid sequences in a same molecule includes, but is not limited to, an oligomeric compound comprising oligonucleotides of the invention (e.g., a hairpin oligo).

As used herein, “internucleoside linkage” is the covalent linkage between adjacent nucleosides in an oligonucleotide. As used herein “modified internucleoside linkage” means any internucleoside linkage other than a phosphodiester internucleoside linkage. A “phosphorothioate internucleoside linkage” is a modified internucleoside linkage in which one of the non-bridging oxygen atoms of a phosphodiester internucleoside linkage is replaced with a sulfur atom.

As used herein, “inverted nucleoside” means a nucleoside having a 3′ to 3′ and/or 5′ to 5′ internucleoside linkage, as shown herein. As used herein, “inverted sugar moiety” means the sugar moiety of an inverted nucleoside or an abasic sugar moiety having a 3′ to 3′ and/or 5′ to 5′ internucleoside linkage.

As used herein, “linked nucleosides” are nucleosides that are connected in a contiguous sequence (i.e., nucleosides immediately adjacent to one another, no additional nucleosides are presented between those that are linked).

As used herein, “linker-nucleoside” means a nucleoside that links, either directly or indirectly, an oligonucleotide to a conjugate moiety. When present in a compound, linker-nucleosides are located within the conjugate linker of a compound of the invention. Linker-nucleosides are not considered part of the oligonucleotide portion of a compound even if they are contiguous with the oligonucleotide.

As used herein, “mismatch” or “non-complementary” means a nucleobase of a first nucleic acid sequence that is not complementary with the corresponding nucleobase of a second nucleic acid sequence when the first and second nucleic acid sequences are aligned in opposing directions.

As used herein, “modified nucleoside” means a nucleoside comprising a modified nucleobase and/or a modified sugar moiety. Modified nucleosides include abasic nucleosides.

As used herein, “modified sugar moiety” means a furanosyl sugar moiety of a nucleoside other than β-D-ribosyl sugar moiety (the sugar moiety of unmodified RNA).

As used herein, “motif” means the pattern of unmodified and/or modified sugar moieties, nucleobases, and/or internucleoside linkages, in an oligonucleotide.

As used herein, “non-bicyclic modified sugar moiety” means a modified sugar moiety that comprises a modification, such as a substituent, that does not form a bridge between two atoms of the sugar to form a second ring.

As used herein, “nucleobase” means an unmodified nucleobase or a modified nucleobase. As used herein an “unmodified nucleobase” is unmodified adenine (A), unmodified thymine (T), unmodified cytosine (C), unmodified uracil (U), or unmodified guanine (G). As used herein, a “modified nucleobase” is a group of atoms other than unmodified A, T, C, U, or G capable of pairing with at least one unmodified nucleobase. A 5-methylcytosine and hypoxanthine are modified nucleobases.

As used herein, “the nucleobase sequence of” a reference SEQ ID NO, refers only to the nucleobase sequence provided in such SEQ ID NO and therefore, unless otherwise indicated, includes compounds wherein each sugar moiety and each internucleoside linkage, independently, is modified or unmodified, irrespective of the presence or absence of modifications, indicated in the referenced SEQ ID NO.

As used herein, “nucleobase sequence” means the order of contiguous nucleobases in a nucleic acid or oligonucleotide independent of any sugar or internucleoside linkage modification.

As used herein, “nucleoside overhang” or “overhang” refers to unpaired nucleosides at either or both ends of an oligomeric duplex formed by hybridization of two nucleotide sequences.

As used herein, “nucleoside” means a compound or fragment of a compound comprising a nucleobase and a sugar moiety. The nucleobase and the sugar moiety of each nucleoside are each, independently, unmodified or modified. Notwithstanding the foregoing and as described herein, a modified nucleoside includes abasic nucleosides.

As used herein, “oligomeric compound” means an oligonucleotide and optionally one or more additional features, e.g., a conjugate group or terminal group. A “singled-stranded oligomeric compound” is an unpaired oligomeric compound. A “double-stranded oligomeric compound” is a paired oligomeric compound which may be formed by a single oligonucleotide of an oligomeric compound forming a hairpin structure resulting in at least a region of double strand oligonucleotide sequence; or by two oligonucleotides, each of a separate oligomeric compound, either or both of which optionally include additional features, and wherein the oligonucleotide sequences, or portions thereof, pair to form a double strand, an oligomeric duplex. The term “oligomeric duplex” means a duplex formed by two oligomeric compounds, at least a portion of which are complementary to and hybridize to each other. Each oligomeric compound of an oligomeric duplex may be referred to as a “duplexed oligomeric compound.”

As used herein, “oligonucleotide” means a strand of linked nucleosides connected via internucleoside linkages, wherein each nucleoside and/or each internucleoside linkage may be independently modified or unmodified. Unless otherwise indicated, oligonucleotides consist of 12-50 linked nucleosides. As used herein, “modified oligonucleotide” means an oligonucleotide comprising one or more modified nucleosides and/or having one or more modified internucleoside linkages. As used herein, “unmodified oligonucleotide” means an oligonucleotide that does not comprise any nucleoside modifications or internucleoside linkage modifications. An oligonucleotide may be paired with a second oligonucleotide that is complementary to the oligonucleotide or it may be unpaired. As used herein, “single-stranded” means a nucleic acid (including but not limited to an oligonucleotide) that is unpaired and is not part of a duplex. Single-stranded nucleic acids (e.g., oligonucleotides) are capable of hybridizing with complementary nucleic acids to form duplexes, at which point they are no longer single-stranded. As used herein, “duplex” means a structure formed by two separate nucleic acid molecules or portions thereof (e.g., two separate oligonucleotides), at least a portion of which are complementary and that are hybridized to one another, but are not covalently bonded to one another. As used herein, “double-stranded” refers to a region of hybridized oligonucleotide(s). A double-stranded oligonucleotide means either two separate oligonucleotides that are hybridized to one another (a duplex) or a single molecule (e.g., oligonucleotide) that has folded onto itself (e.g., a hairpin structure). In certain embodiments, such double-strand results from hybridization of an oligonucleotide (or portion thereof) to a target region of a transcript. In certain embodiments, a double-strand results from hybridization of two oligonucleotides (or portions thereof) to one another. In certain embodiments, the hybridized regions are portions (including the entirety) of two separate molecules (e.g., no covalent bond connects the two complementary strands together). In certain embodiments, the hybridized regions are portions of the same molecule that have hybridized (e.g., a hairpin structure).

As used herein “pharmaceutical composition” means a mixture of substances suitable for administering to a subject. For example, a pharmaceutical composition may comprise a compound of the invention (e.g., an oligomeric compound, modified oligonucleotide, oligomeric duplex, or antisense compound) and a sterile aqueous solution. In certain embodiments, a pharmaceutical composition shows activity in certain cell lines. As used herein, “pharmaceutically acceptable carrier or diluent” means an ingredient in a pharmaceutical composition suitable for use in administering to a subject. Typically, a “carrier” or “diluent” lacks pharmacological activity, but is necessary or desirable in preparing a pharmaceutical composition. For example, a diluent in an injected composition can be a liquid, e.g., PBS, or saline solution. Certain carriers or diluents enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water, sterile saline, sterile buffer solution or sterile artificial cerebrospinal fluid.

As used herein, “reduced fluorine content” with respect to a contiguous sequence of linked nucleosides, for example, an oligonucleotide (including, for example an antisense oligonucleotide and a sense oligonucleotide) refers to a contiguous linked sequence of nucleosides in which fewer than 51% of the nucleosides contain a sugar moiety or sugar surrogate that includes a fluorine atom, e.g., a 2′-fluoro sugar moiety or a fluoro hexitol (3′-FHNA) sugar moiety. In certain embodiments, no more than 42%, no more than 40%, no more than 35%, no more than 30%, no more than 25%, no more than 20%, no more than 16%, no more than 15%, no more than 14%, no more than 13%, no more than 12%, no more than 10%, no more than 9%, or no more than 5% of the nucleosides in the contiguous linked sequence of nucleosides contain a sugar moiety containing a fluorine atom. In certain embodiments, none of the nucleosides in the contiguous linked sequence of nucleosides contain a sugar moiety containing a fluorine atom. In some embodiments, fewer than 48%, fewer than 45%, fewer than 42%, fewer than 40%, fewer than 35%, fewer than 30%, fewer than 25%, fewer than 20%, fewer than 15%, fewer than 14%, fewer than 13%, fewer than 12%, fewer than 10%, or fewer than 5%, of the nucleosides in the contiguous linked sequence of nucleosides contain a sugar moiety containing a fluorine atom. “Reduced fluorine content” when referring to the total fluorine content of a double-stranded or duplex nucleic acid refers to a double-stranded or duplex nucleic acid in which fewer than 50% of the total nucleosides (i.e., all the nucleosides contained in both strands) of the nucleic acid contain a sugar moiety containing a fluorine atom. In certain embodiments, no more than 45%, no more than 42%, no more than 40%, no more than 35%, no more than 30%, no more than 27%, no more than 26%, no more than 25%, no more than 22%, no more than 15%, no more than 10%, or no more than 5% of the total nucleosides in the double-stranded or duplex nucleic acid contain a sugar moiety containing a fluorine atom. In certain embodiments, none of the nucleosides in the double-stranded or duplex nucleic acid contain a sugar moiety containing a fluorine atom. In some embodiments, fewer than 42%, fewer than 40%, fewer than 35%, fewer than 30%, fewer than 27%, fewer than 26%, fewer than 22%, fewer than 20%, fewer than 15%, fewer than 10%, or fewer than 5% of the total nucleosides in the double-stranded or duplex nucleic acid contain a sugar moiety containing a fluorine atom.

As used herein, “RNAi agent” means an antisense compound that acts, at least in part, through RISC or Ago2 to modulate a target nucleic acid and/or protein encoded by a target nucleic acid. RNAi agents include, but are not limited to double-stranded siRNA, single-stranded RNAi (ssRNAi, and microRNA, including microRNA mimics. RNAi agents may comprise conjugate groups and/or terminal groups. In certain embodiments, an RNAi agent modulates the amount and/or activity, of a target nucleic acid. The term RNAi agent excludes antisense compounds that act through RNase H.

As used herein, “stabilized phosphate group” means a 5′-phosphate analog that is metabolically more stable than a 5′-phosphate as naturally occurs on DNA or RNA.

As used herein, “standard cell assay” means the assays described in the Examples and reasonable variations thereof.

As used herein, “stereorandom” or “stereorandom chiral center” in the context of a population of molecules of identical molecular formula means a chiral center that is not controlled during synthesis, or enriched following synthesis, for a particular absolute stereochemical configuration. It is understood that a stereorandom chiral center may not be racemic because one absolute configuration predominates following synthesis, e.g., due to steric and electronic interactions of reagents with the reactant molecule. In certain embodiments, the stereorandom chiral center is at the phosphorous atom of a stereorandom phosphorothioate or mesyl phosphoramidate internucleoside linkage.

As used herein, “subject” means a human or non-human animal. In certain embodiments, the subject is a human.

As used herein, “sugar moiety” means an unmodified sugar moiety or a modified sugar moiety. As used herein, “unmodified sugar moiety” means a 2′-OH(H) β-D-ribosyl sugar moiety, as found in RNA (an “unmodified RNA sugar moiety”). Unmodified sugar moieties have one hydrogen at each of the 1′, 3′, and 4′ positions, an oxygen at the 3′ position, a hydrogen and an OH at the 2′ position, and two hydrogens at the 5′ position. Modified sugar moieties differ from unmodified RNA by having different substituent(s) (e.g., 2′-F, 2′-MOE, cEt, etc.,), having a 2′-deoxy sugar moiety and/or having different stereochemistry (e.g., a 2′-α-L-deoxyribosyl sugar moiety). In certain embodiments, a modified sugar moiety is a modified ribosyl sugar moiety. In certain embodiments, modified sugar moieties differ from unmodified RNA by having both different chemistry (e.g., different substituent(s), 2′-deoxy sugar moiety) and different stereochemistry.

As used herein, “sugar surrogate” means a moiety that can link a nucleobase to another group, such as an internucleoside linkage, conjugate group, or terminal group in an oligonucleotide, but which is not a furanosyl sugar moiety or a bicyclic sugar moiety or a non-bicyclic sugar moiety. Sugar surrogates may comprise one or more substitutions corresponding to those of other types of modified sugar moieties. Modified nucleosides comprising sugar surrogates can be incorporated into one or more positions within an oligonucleotide and such oligonucleotides are capable of hybridizing to complementary oligonucleotide compounds or target nucleic acids. Examples of sugar surrogates include GNA (glycol nucleic acid), FHNA (fluoro hexitol nucleic acid), morpholino, and other structures described herein and known in the art for use in oligonucleotides.

As used herein, “symptom” means any manifestation, indication, sign, evidence, or physical feature or test result that indicates the existence or extent of a disease or disorder. Symptoms include subjective and objective indicia of a disease and may be perceived, experienced, detected, observed, measured, and/or quantified. In certain embodiments, a symptom is apparent to a subject or to a medical professional examining or testing said subject. In certain embodiments, a symptom is apparent upon invasive diagnostic testing, including, but not limited to, post-mortem tests. A symptom may be an absence of a feature, such as failing to reach expected developmental milestones.

As used herein, “target nucleic acid” means a PLN nucleic acid that an antisense compound is designed to affect. As used herein, “target RNA” means a PLN RNA transcript and includes pre-mRNA and/or mRNA unless otherwise specified or specifically relevant (e.g., intron sequence in pre-mRNA). As used herein, “target region” means a portion of a PLN target nucleic acid to which a compound of the invention (e.g., a modified oligonucleotide, an antisense compound) is designed to hybridize.

As used herein, “therapeutic index” refers to a quantitative relationship, e.g., ratio, between the concentration (or dose) at which a compound becomes toxic or induces unacceptable adverse effects (or the highest concentration or dose at which a compound is not toxic, or is tolerated, before it becomes toxic or induces unacceptable adverse effects) to a subject and the concentration (or dose) at which the compound is pharmacologically effective, or produces the desired efficacy. These concentrations or doses can be determined using various parameters depending in part on the indication for which the compound is being used as a therapeutic agent. The higher the therapeutic index, the more favorable the safety profile is for the compound. The therapeutic index provides a range of effective concentrations (or doses) for a compound to achieve effective results without unacceptable adverse events. A therapeutic index can be increased by raising the concentration (or dose) at which a compound becomes toxic or induces adverse effects and/or lowering the concentration (or dose) at which a compound is effective.

As used herein, “treating” means improving, or preventing, or delaying development or progression of a subject's disease, disorder or condition or injury by administering a compound or therapeutic agent (e.g., a modified oligonucleotide, oligomeric duplex, an antisense compound described herein) to a subject, e.g., a subject having or at risk of developing a disease, disorder or condition or injury. In certain embodiments, treating a subject improves a symptom relative to the same symptom in the absence of the treatment. In certain embodiments, treatment reduces the severity or frequency of a symptom, or delays the onset of a symptom, slows the progression of a symptom, or slows the increase of the severity or slows the frequency (or increase in frequency) of a symptom. In some embodiments, treatment reduces, improves, and/or prevents one or more symptom(s) such that a symptom of the disorder or disease is diminished or no longer apparent.

### EMBODIMENTS

Embodiment 1. An oligomeric duplex comprising a first oligomeric compound and a second oligomeric compound, wherein:
(1) the first oligomeric compound comprises a modified oligonucleotide consisting of 8 to 50 linked nucleosides, wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases of the nucleobase sequence of any one of SEQ ID NOs: 3-314, 627-782, 939-976, 1033-1038, 1254-1255, or 1258-1287, wherein each of the nucleosides of the first modified oligonucleotide independently comprises a modified sugar moiety or sugar surrogate and wherein at least one modified nucleoside and less than 40% of the nucleosides of the first modified oligonucleotide comprises a fluorine; and
(2) the second oligomeric compound comprises a second modified oligonucleotide consisting of 8 to 50 contiguous linked nucleosides comprising a region having a nucleobase sequence complementary to a region of the first modified oligonucleotide, wherein the nucleobase sequence of the second modified oligonucleotide comprises at least 8, at least 9, at least 10, at least 11, at least 12, at least 13, at least 14, at least 15, at least 16, at least 17, at least 18, at least 19, at least 20, or at least 21 contiguous nucleobases of the nucleobase sequence of any one of SEQ ID NOs: 315-626, 783-938, 977-1016, 1027-1032, 1256-1257, or 1288-1290, wherein each of the nucleosides of the second modified oligonucleotide independently comprises a modified sugar moiety or sugar surrogate and wherein less than 40% of the nucleosides of the second modified oligonucleotide comprises a fluorine.

Embodiment 2. The oligomeric duplex of embodiment 1, wherein the first modified oligonucleotide is at least 80%, at least 85%, at least 90%, at least 95%, or at least 99% or 100% complementary to an equal length portion of a PLN nucleic acid, wherein the PLN nucleic acid has the nucleobase sequence of SEQ ID NO: 1 or SEQ ID NO: 2.

Embodiment 3. The oligomeric duplex of embodiment 1 or embodiment 2, wherein each of the nucleosides of the first modified oligonucleotide independently comprises and each of the nucleosides of the second modified oligonucleotide independently comprises (i) a modified sugar moiety or sugar surrogate, or (ii) a sugar moiety independently selected from a 2′-F sugar moiety, a 2′-MOE sugar moiety, a 2′-OMe sugar moiety, a 2′-deoxy sugar moiety, and a 3′-fluoro-hexitol sugar moiety, or (iii) a modified sugar moiety. Embodiment 4. The oligomeric duplex of any one of embodiments 1-3, wherein fewer than 35%, fewer than 30%, fewer than 25%, fewer than 20%, fewer than 15%, fewer than 14%, fewer than 13%, fewer than 12%, fewer than 10%, fewer than 5%, or fewer than 1% of the nucleosides in the first modified oligonucleotide comprises a fluorine.

Embodiment 5. The oligomeric duplex of any one of embodiments 1-4, wherein no more than 1 nucleoside, no more than 2 nucleosides, no more than 3 nucleosides, or no more than 4 nucleosides in the first modified oligonucleotide comprise a modified sugar moiety comprising a fluorine or a sugar surrogate comprising a fluorine.

Embodiment 6. The oligomeric duplex of any one of embodiments 1-5, wherein no more than 1 nucleoside, no more than 2 nucleosides, or no more than 3 nucleosides within the sequence of the first modified oligonucleotide between and including the third and twenty-first nucleosides counting from the 5′ end of the modified oligonucleotide comprise a modified sugar moiety comprising a fluorine or a sugar surrogate comprising a fluorine.

Embodiment 7. The oligomeric duplex of any one of embodiments 1-6, wherein one or more nucleoside(s) of the first modified oligonucleotide comprising i) a modified sugar moiety comprising a fluorine or ii) a sugar surrogate comprising a fluorine is/are independently selected from:
i. the second nucleoside counting from the 5′ end,
ii. the fourteenth and sixteenth nucleosides counting from the 5′ end, or
iii. the second, fourteenth and sixteenth nucleosides counting from the 5′ end; wherein each i) modified sugar moiety comprising a fluorine or ii) sugar surrogate comprising a fluorine is independently a 2′-fluoro sugar moiety or a 3′-fluoro-hexitol sugar moiety.

Embodiment 8. The oligomeric duplex of any one of embodiments 1-7, wherein no more than one of the sugar moieties comprising a fluorine in the first modified oligonucleotide is a 3′-fluoro-hexitol sugar moiety.

Embodiment 9. The oligomeric duplex of any one of embodiments 1-8, wherein one or more nucleosides of the first modified oligonucleotide is a 2′-deoxynucleoside.

Embodiment 10. The oligomeric duplex of embodiment 9, wherein the one or more 2′-deoxynucleosides is one or more nucleosides in a region of the sequence of the first modified oligonucleotide between and including the fifth nucleoside to the sixteenth nucleoside counting from the 5′ end of the first modified oligonucleotide.

Embodiment 11. The oligomeric duplex of embodiment 10, wherein the one or more 2′-deoxynucleosides is in a region of the sequence of the first modified oligonucleotide that is any of the fifth, and/or sixth, and/or seventh nucleosides or that is any of the fourteenth, and/or fifteenth, and/or sixteenth nucleosides counting from the 5′ end of the first modified oligonucleotide.

Embodiment 12. The oligomeric duplex of any one of embodiments 9-11, wherein fewer than 20%, or fewer than 15%, of the nucleosides of the first modified oligonucleotide comprises a fluorine.

Embodiment 13. The oligomeric duplex of any one of embodiments 9-12, wherein the one or more 2′-deoxynucleosides is the fifth, and/or sixth, and/or seventh nucleoside(s) counting from the 5′ end of the first modified oligonucleotide.

Embodiment 14. The oligomeric duplex of any one of embodiments 9-13, wherein only one or only two nucleosides of the first modified oligonucleotide are 2′-deoxynucleosides.

Embodiment 15. The oligomeric duplex of embodiment 14, wherein the two 2′-deoxynucleosides are the fifth and seventh nucleosides, or the fourteenth and sixteenth nucleosides, counting from the 5′ end of the first modified oligonucleotide.

Embodiment 16. The oligomeric duplex of embodiment 14, wherein the only 2′-deoxynucleoside is the sixth or sixteenth nucleoside counting from the 5′ end of the first modified oligonucleotide.

Embodiment 17. The oligomeric duplex of any one of embodiments 1-8, wherein none of the nucleosides of the first modified oligonucleotide is a 2′-deoxynucleoside.

Embodiment 18. The oligomeric duplex of any one of embodiments 1-17, wherein the first oligomeric compound comprises a two-nucleoside overhang comprising the 3′-terminal nucleoside and the nucleoside immediately 5′ of the 3′-terminal nucleoside of the first modified oligonucleotide.

Embodiment 19. The oligomeric duplex of embodiment 18, wherein the overhang nucleosides are two modified adenosine (AA) nucleosides, two modified uridine (UU) nucleosides, two modified inosine (II) nucleosides, or two modified nucleosides wherein one is inosine and one is adenosine (AI or IA).

Embodiment 20. The oligomeric duplex of any one of embodiments 1-19, wherein one or more of the nucleosides of the first modified oligonucleotide independently comprises a sugar surrogate or a bicyclic sugar moiety.

Embodiment 21. The oligomeric duplex of embodiment 20, wherein the bicyclic sugar moiety comprises a 2′-4′ bridge selected from —O—CH₂—; and —O—CH(CH₃)—.

Embodiment 22. The oligomeric duplex of any one of embodiments 1-21, wherein one or more of the nucleosides of the first modified oligonucleotide comprises a 2′-OMe sugar moiety.

Embodiment 23. The oligomeric duplex of embodiment 22, wherein at least 10%, at least 20%, at least 30%, at least 40%, at least 50% or at least 60% of the nucleosides of the first modified oligonucleotide comprise a 2′-OMe sugar moiety.

Embodiment 24. The oligomeric duplex of embodiment 22 or embodiment 23, wherein the one or more nucleosides comprising a 2′-OMe sugar moiety are in a region of the sequence of the first modified oligonucleotide between and including the third and twenty-first nucleosides counting from the 5′ end of the first modified oligonucleotide, and/or wherein the first two nucleosides counting from the 5′ end of the first modified oligonucleotide and/or the first two nucleosides counting from the 3′ end of the first modified oligonucleotide are modified nucleosides independently comprising a sugar surrogate or a modified sugar moiety other than a 2′-OMe sugar moiety.

Embodiment 25. The oligomeric duplex of embodiment 22 or embodiment 23, wherein the 3′-terminal nucleoside of the first modified oligonucleotide comprises a 2′-OMe sugar moiety and/or the nucleoside immediately 5′ of the 3′-terminal nucleoside of the first modified oligonucleotide comprises a 2′-OMe sugar moiety.

Embodiment 26. The oligomeric duplex of any one of embodiments 1-25, wherein one or more of the nucleosides of the first modified oligonucleotide comprise a 2′-MOE sugar moiety.

Embodiment 27. The oligomeric duplex of embodiment 26, wherein the 5′- and/or 3′-terminal nucleosides of the first modified oligonucleotide comprise a 2′-MOE sugar moiety.

Embodiment 28. The oligomeric duplex of any one of embodiments 1-27, wherein the nucleoside immediately 5′ of the 3′-terminal nucleoside of the first modified oligonucleotide comprises a 2′-MOE sugar moiety.

Embodiment 29. The oligomeric duplex of any one of embodiments 1-24 and 26-28, wherein the 5′- and 3′-terminal nucleosides of the first modified oligonucleotide comprise a 2′-MOE sugar moiety and the nucleoside immediately 5′ of the 3′-terminal nucleoside of the first modified oligonucleotide comprises a 2′-MOE sugar moiety.

Embodiment 30. The oligomeric duplex of any one of embodiments 1-29, wherein the first oligomeric compound comprises a stabilized phosphate group attached to the 5′-terminal nucleoside of the first modified oligonucleotide.

Embodiment 31. The oligomeric duplex of embodiment 30, wherein the stabilized phosphate group comprises a cyclopropyl phosphonate or a vinyl phosphonate.

Embodiment 32. The oligomeric duplex of any one of embodiments 1-31, wherein the duplex comprises a conjugate group comprising a conjugate moiety and a conjugate linker.

Embodiment 33. The oligomeric duplex of embodiment 32, wherein the conjugate group comprises a cell-targeting moiety.

Embodiment 34. The oligomeric duplex of embodiment 32, wherein the conjugate moiety comprises an active drug substance, an aliphatic chain, a lipid, a peptide, a protein, a hydrocarbon, a polyamine, a polyamide, a polyether, a thioether, an aptamer, an antibody, an antibody fragment, a vitamin, a fatty acid, a carbohydrate, an intercalator, or a reporter molecule.

Embodiment 35. The oligomeric duplex of embodiment 32, wherein the conjugate group comprises a C22 alkyl, C20 alkyl, C17 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, or C5 alkyl, wherein the alkyl chain optionally has one or more unsaturated bonds.

Embodiment 36. The oligomeric duplex of embodiment 32, wherein the conjugate group comprises a 6-palmitamidohexyl moiety or a 2-(hydroxymethyl)-6-palmitamidohexyl moiety.

Embodiment 37. The oligomeric duplex of embodiment 32, wherein the conjugate group comprises a conjugate moiety that binds type 1 transferrin receptor (TfR1).

Embodiment 38. The oligomeric duplex of embodiment 37, wherein the conjugate moiety is selected from an antibody or fragment thereof, a protein or peptide, and an aptamer capable of binding TfR1.

Embodiment 39. The oligomeric duplex of embodiment 38, wherein the conjugate moiety is a cyclic protein or cyclic peptide.

Embodiment 40. The oligomeric duplex of embodiment 37, wherein the conjugate group comprises a bicycle ligand and a conjugate linker.

Embodiment 41. The oligomeric duplex of embodiment 40, wherein the bicycle ligand comprises a peptide consisting of 13-22 linked amino acids or amino acid mimetics and a molecular scaffold, wherein each of a first, a second, and a third amino acid of the peptide comprises a reactive group, each of which separately forms a bond with the molecular scaffold, thereby forming two peptide loops attached to the molecular scaffold.

Embodiment 42. The oligomeric duplex of embodiment 41, wherein the peptide has an amino acid sequence selected from any one of SEQ ID NOs: 1062-1253 and wherein each cysteine forms a covalent bond with the molecular scaffold 1,1′,1″-(1,3,5-triazinane-1,3,5-triyl)tris(2-bromoethanone) (TATB) or the molecular scaffold 1,1′,1″-(1,3,5-triazinane-1,3,5-triyl)triprop-2-en-1-one (TATA).

Embodiment 43. The oligomeric duplex of embodiment 41, wherein the bicycle ligand or conjugate group has the structure:

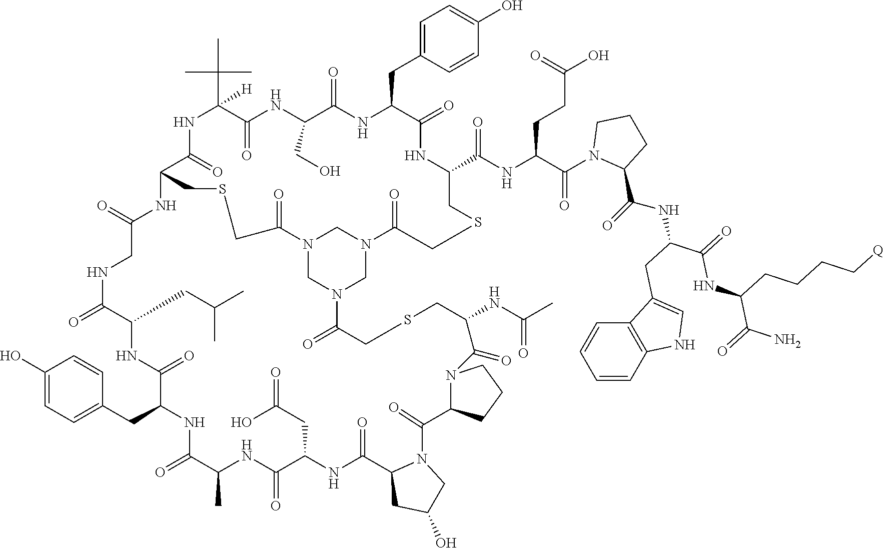

or a salt thereof, wherein Q is N₃ (BCY17901, SEQ ID NO: 1045), NH₂ (BCY21757, SEQ ID NO: 1202), SH (BCY21758, SEQ ID NO: 1203), a conjugate linker, or a conjugate linker covalently connected to an oligonucleotide.

Embodiment 44. The oligomeric duplex of any one of embodiments 32-43, wherein the conjugate group is conjugated to the second modified oligonucleotide.

Embodiment 45. The oligomeric duplex of any one of embodiments 32-43, wherein the conjugate group is attached to the 5′ end or 3′ end of the second modified oligonucleotide.

Embodiment 46. The oligomeric duplex of any one of embodiments 32-43, wherein the conjugate group is attached to the 5′-terminal nucleoside or the 3′-terminal nucleoside of the second modified oligonucleotide.

Embodiment 47. The oligomeric duplex of any one of embodiments 32-43, wherein the conjugate group is attached to the 5′-terminal nucleoside of the second modified oligonucleotide.

Embodiment 48. The oligomeric duplex of any one of embodiments 32-43, wherein the conjugate group is attached to the 3′-terminal nucleoside of the second modified oligonucleotide.

Embodiment 49. The oligomeric duplex of any one of embodiments 32-48, wherein the conjugate linker of the conjugate group consists of a single bond.

Embodiment 50. The oligomeric duplex of any one of embodiments 32-48, wherein the conjugate linker of the conjugate group is cleavable.

Embodiment 51. The oligomeric duplex of any one of embodiments 32-50, wherein the conjugate linker comprises 1 to 3 linker-nucleosides.

Embodiment 52. The oligomeric duplex of any one of embodiments 32-48, wherein the conjugate linker comprises a (bicyclo[6.1.0]nonyne)-formyl (BCN) moiety.

Embodiment 53. The oligomeric duplex of any one of embodiments 32-48, wherein the conjugate linker comprises a 6-(BCN-carbamate)-2-(hydroxymethyl)hexyl phosphoryl moiety or a 6-(BCN-carbamate)hexyl phosphoryl moiety.

Embodiment 54. The oligomeric duplex of embodiment 41, wherein the conjugate group has the structure:

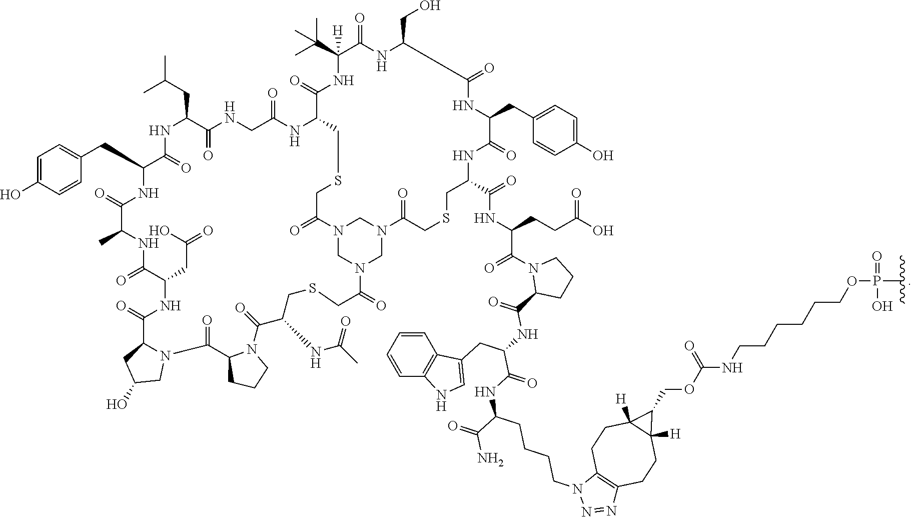

Embodiment 55. The oligomeric duplex of embodiment 41, wherein the conjugate group has the structure:

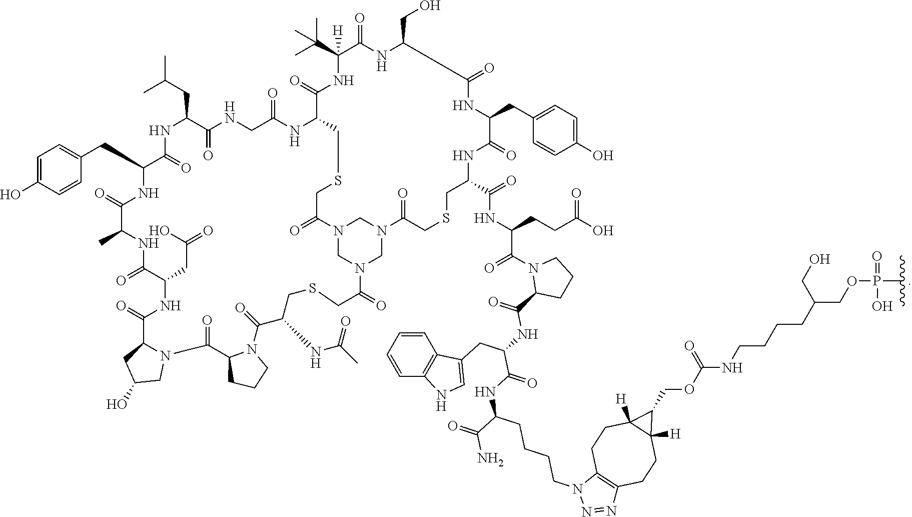

Embodiment 56. The oligomeric duplex of any one of embodiments 1-55, wherein, the nucleobase sequence of the first modified oligonucleotide is at least 80%, at least 85%, at least 90%, at least 95%, or at least 9900 or 100% complementary to an equal length portion within nucleobases selected from any one of 295-316, 296-317, 297-318, 298-320, 299-320, 300-321, 301-322, 302-324, 303-324, 304-325, 304-326, 305-327, 306-325, 306-328, 307-328, 308-329, 309-330, 310-331, 311-332, 312-333, 313-334, 314-335, 315-336, 316-337, 317-339, 318-339, 319-340, 320-341, 321-342, 322-344, 323-345, 324-345, 324-346, 325-345, 325-347, 326-347, 327-348, 328-349, 329-350, 330-351, 331-352, 332-353, 333-354, 334-355, 505-526, 506-528, 507-529, 508-530, 509-531, 510-531, 511-532, 512-533, 513-535, 514-535, 514-536, 515-536, 516-535, 516-537, 517-538, 518-539, 519-541, 520-541, 521-542, 522-544, 523-544, 524-546, 535-556, 536-557, 537-558, 538-559, 539-560, 540-561, 541-563, 542-563, 543-564, 544-565, 545-566, 546-567, 547-568, 548-570, 549-571, 550-572, 551-572, 552-574, 553-574, 554-575, 555-577, 556-578, 557-579, 558-580, 559-580, 560-581, 561-582, 562-583, 563-584, 595-616, 596-618, 597-618, 598-620, 599-621, 600-622, 601-623, 602-623, 603-624, 604-625, 605-627, 606-628, 607-628, 608-629, 609-630, 610-631, 611-632, 612-633, 613-635, 665-687, 666-687, 667-689, 668-689, 669-690, 670-691, 671-692, 672-694, 673-694, 674-695, 675-696, 676-697, 677-698, 678-700, 679-701, 680-702, 681-703, 682-703, 683-704, 684-705, 685-706, 686-708, 687-708, 688-709, 689-710, 690-711, 691-712, 692-714, 693-715, 1675-1696, 1676-1698, 1677-1698, 1678-1699, 1679-1700, 1680-1702, 1681-1702, 1682-1703, 1683-1705, 1684-1705, 1685-1706, 1686-1707, 1687-1709, 1688-1709, 1689-1710, 1690-1712, 1691-1713, 1692-1714, or 1693-1714 of SEQ ID NO: 1.

Embodiment 57. The oligomeric duplex of any one of embodiments 1-55, wherein the nucleobase sequence of the first modified oligonucleotide is at least 80%, at least 85%, at least 90%, at least 95%, or 100% complementary to an equal length portion within the sequence of nucleobases 304-326, 306-325, 324-346, 325-345, 514-536, or 516-535 of SEQ ID NO: 1.

Embodiment 58. The oligomeric duplex of any one of embodiments 1-55, wherein the nucleobase sequence of the first modified oligonucleotide comprises at least 16, at least 17, at least 18, at least 19, at least 20, at least 21, at least 22, or at least 23 contiguous nucleobases of any one of the nucleobase sequences of SEQ ID NOs: 3-314, 627-782, 939-976, 1033-1038, 1254-1255, or 1258-1287.

Embodiment 59. The oligomeric duplex of any one of embodiments 1-55, wherein the nucleobase sequence of the first modified oligonucleotide comprises the nucleobase sequence of any one of SEQ ID NOs: 3-314, 627-782, 939-976, 1033-1038, 1254-1255 or 1258-1287.

Embodiment 60. The oligomeric duplex of any one of embodiments 1-55, wherein the nucleobase sequence of the first modified oligonucleotide consists of the nucleobase sequence of any one of SEQ ID NOs: 3-314, 627-782, 939-976, 1033-1038, 1254-1255 or 1258-1287.

Embodiment 61. The oligomeric duplex of any one of embodiments 1-55, wherein the nucleobase sequence of the first modified oligonucleotide comprises at least 18, at least 19, at least 20, at least 21, at least 22, or at least 23 contiguous nucleobases of any one of the nucleobase sequences of SEQ ID NOs: 29, 171, 176, 967, 968, 971, 972, 974, 975, 1254, 1255, 1033-1038, 1254-1255, or 1258-1287.

Embodiment 62. The oligomeric duplex of any one of embodiments 1-55, wherein the nucleobase sequence of the first modified oligonucleotide comprises the nucleobase sequence of any one of SEQ ID NOs: 29,171,176, 967, 968, 971, 972, 974, 975, 1254, 1255, or 1033-1038, 1254-1255, or 1258-1287.

Embodiment 63. The oligomeric duplex of any one of embodiments 1-55, wherein the nucleobase sequence of the first modified oligonucleotide consists of the nucleobase sequence of any one of SEQ ID NOs: 29,171,176, 967, 968, 971, 972, 974, 975, 1254, 1255, 1033-1038, 1254-1255, or 1258-1287.

Embodiment 64. The oligomeric duplex of any one of embodiments 1-63, wherein the first modified oligonucleotide and the second modified oligonucleotide each independently consist of 18 to 30, 18 to 25, 18 to 24, 18 to 23, 18 to 22, 18 to 21, 18 to 19, 19 to 30, 19 to 25, 19 to 24, 19 to 23, 19 to 21, 19 to 20, 20 to 30, 20 to 25, 20 to 24, 20 to 23, 20 to 22, 20 to 21, 21 and 19, 21 and 20, 23 and 21, 23 and 22, or 22 and 21 linked nucleosides.

Embodiment 65. The oligomeric duplex of any one of embodiments 1-64, wherein the first modified oligonucleotide consists of 23, 22, or 21 nucleosides.

Embodiment 66. The oligomeric duplex of any one of embodiments 1-65, wherein the second modified oligonucleotide consists of 21 nucleosides.

Embodiment 67. The oligomeric duplex of any one of embodiments 1-65, wherein the first modified oligonucleotide consists of 23 nucleosides and the second modified oligonucleotide consists of 21 nucleosides.

Embodiment 68. The oligomeric duplex of any one of embodiments 1-65, wherein the first modified oligonucleotide consists of 22 nucleosides and the second modified oligonucleotide consists of 21 nucleosides.

Embodiment 69. The oligomeric duplex of any one of embodiments 1-65, wherein the first modified oligonucleotide consists of 21 nucleosides and the second modified oligonucleotide consists of 21 nucleosides.

Embodiment 70. The oligomeric duplex of any one of embodiments 1-69, wherein the first modified oligonucleotide has a sugar motif (5′ to 3′) selected from among: efyyydyyyyyyyfyfyyyyyee, efyyydyyyyyyyfyfyyyyyyy, e[FHNA]yyydyyyyyyyfyfyyyyyee, e[FHNA]yyydyyyyyyyfyfyyyyyyy, efyydydyyyyyyfyfyyyyyee, efyydydyyyyyyfyfyyyyyyy, e[FHNA]yydydyyyyyyfyfyyyyyee, e[FHNA]yydydyyyyyyfyfyyyyyyy, efyyyfyyyyyyyfyfyyyyyyy, efyyyfyyyyyyyfyfyyyyyey, efyyyyyyyyyyyfyfyyyyyee, efyyyfyyyyyyyfyfyyyyyee, efyyyyyyyyyyyfyyyyyyyee, efyyyyyyyyyyyfyyyyyyyyy, e[FHNA]yyyfyyyyyyyfyfyyyyyee, e[FHNA]yyyfyyyyyyyfyfyyyyyyy, efyyydyyyyyyydydyyyyyee, efyyydyyyyyyydydyyyyyyy, efyydydyyyyyyfyfyyyyyyy, efyyyfyyyyyyyfyyyyyyyee, efyyyfyyyyyyyfyyyyyyyyy, efyyyyyyyyyyyfyfyyyyyee, efyyyyyyyyyyyfyfyyyyyyy, e[FHNA]yydydyyyyfyfyfyyyyyyy, efyyyfyyyyyyyfydyyyyyyy, efyyyfyyyyyyyfydyyyyyee, efyydydyyyyyyfyfyyyyye, efyydydyyyyyyfyfyyyyyy, efyyydyyyyyyyfyfyyyyye, efyyydyyyyyyyfyfyyyyyy, efyyyyyyyyyyyfyfyyyyye, efyyyyyyyyyyyfyfyyyyyy, efyydydyyyyyyfyfyyyyy, efyyydyyyyyyyfyfyyyyy, efyyyyyyyyyyyfyfyyyyy, efyyyfyyyyyyyfyfyyyee, and efyyyfyyyyyyyfyfyyyyy, wherein each “d” represents a 2′-deoxy sugar moiety, each “y” represents a 2′-OMe sugar moiety, each “e” represents a 2′-MOE sugar moiety, each “f” represents a 2′-fluoro sugar moiety, and each “[FHNA]” represents a 3′-fluoro-hexitol sugar moiety.

Embodiment 71. The oligomeric duplex of any one of embodiments 1-69, wherein the first modified oligonucleotide has a sugar motif (5′ to 3′) selected from among: efyydydyyyyyyfyfyyyyyee, efyyydyyyyyyyfyfyyyyyee, efyydydyyyyyyfyfyyyyyyy, e[FHNA]yydydyyyyfyfyfyyyyyyy, efyyyyyyyyyyyfyfyyyyyee, efyyydyyyyyyyfyfyyyyyyy, efyydydyyyyyyfyfyyyyye, efyydydyyyyyyfyfyyyyyy, efyyydyyyyyyyfyfyyyyye, efyyydyyyyyyyfyfyyyyyy, efyyyyyyyyyyyfyfyyyyye, efyyyyyyyyyyyfyfyyyyyy, efyydydyyyyyyfyfyyyyy, efyyydyyyyyyyfyfyyyyy, and efyyyyyyyyyyyfyfyyyyy.

Embodiment 72. The oligomeric duplex of any one of embodiments 1-71, wherein the first modified oligonucleotide comprises at least one modified internucleoside linkage.

Embodiment 73. The oligomeric duplex of embodiment 72, wherein at least one modified internucleoside linkage is a phosphorothioate or mesyl phosphoramidate internucleoside linkage.

Embodiment 74. The oligomeric duplex of embodiment 72, wherein fewer than 50%, fewer than 40%, fewer than 30%, fewer than 20%, or fewer than 10% of the internucleoside linkages of the first modified oligonucleotide are modified internucleoside linkages.

Embodiment 75. The oligomeric duplex of any one of embodiments 1-74, wherein each internucleoside linkage of the first modified oligonucleotide is independently selected from a phosphodiester internucleoside linkage and a phosphorothioate internucleoside linkage.

Embodiment 76. The oligomeric duplex of embodiment 75, wherein the internucleoside linkages between the first and second nucleosides and the second and third nucleosides counting from the 5′ end of the first modified oligonucleotide are modified internucleoside linkages.

Embodiment 77. The oligomeric duplex of embodiment 75, wherein the internucleoside linkages between the first and second nucleosides and the second and third nucleosides counting from the 3′ end of the first modified oligonucleotide are modified internucleoside linkages.

Embodiment 78. The oligomeric duplex of embodiment 75, wherein the internucleoside linkages between the first and second nucleosides and the second and third nucleosides counting from the 5′ end of the first modified oligonucleotide, and the internucleoside linkages between the first and second nucleosides and the second and third nucleosides counting from the 3′ end of the first modified oligonucleotide are modified internucleoside linkages.

Embodiment 79. The oligomeric duplex of any one of embodiments 72-78, wherein the modified internucleoside linkages are phosphorothioate internucleoside linkages.

Embodiment 80. The oligomeric duplex of embodiment 78 or embodiment 79, wherein all other internucleoside linkages in the first modified oligonucleotide are phosphodiester internucleoside linkages.

Embodiment 81. The oligomeric duplex of embodiment 80, wherein the first modified oligonucleotide has an internucleoside linkage motif of ssooooooooooooooooooss and a sugar motif (5′ to 3′) selected from among: efyydydyyyyyyfyfyyyyyee, efyyydyyyyyyyfyfyyyyyee, efyydydyyyyyyfyfyyyyyyy, e[FHNA]yydydyyyyfyfyfyyyyyyy, efyyyyyyyyyyyfyfyyyyyee and efyyydyyyyyyyfyfyyyyyyy; wherein each “d” represents a 2′-deoxy sugar moiety, each “y” represents a 2′-OMe sugar moiety, each “e” represents a 2′-MOE sugar moiety, each “f” represents a 2′-fluoro sugar moiety, each “[FHNA]” represents a 3′-fluoro-hexitol sugar moiety, each “o” represents a phosphodiester internucleoside linkage and each “s” represents a phosphorothioate internucleoside linkage.

Embodiment 82. The oligomeric duplex of embodiment 72, wherein: 1) the first modified oligonucleotide has an internucleoside linkage motif of ssoooooooooooooooooos and a sugar motif (5′ to 3′) selected from among: efyydydyyyyyyfyfyyyyye, efyydydyyyyyyfyfyyyyyy, efyyydyyyyyyyfyfyyyyye, efyyydyyyyyyyfyfyyyyyy, efyyyyyyyyyyyfyfyyyyye, and efyyyyyyyyyyyfyfyyyyyy; or 2) the first modified oligonucleotide has an internucleoside linkage motif of ssoooooooooooooooooo and a sugar motif (5′ to 3′) selected from among: efyydydyyyyyyfyfyyyyy, efyyydyyyyyyyfyfyyyyy, and efyyyyyyyyyyyfyfyyyyy; wherein each “d” represents a 2′-deoxy sugar moiety, each “y” represents a 2′-OMe sugar moiety, each “e” represents a 2′-MOE sugar moiety, each “f” represents a 2′-fluoro sugar moiety, each “o” represents a phosphodiester internucleoside linkage and each “s” represents a phosphorothioate internucleoside linkage.

Embodiment 83. The oligomeric duplex of any one of embodiments 1-82, wherein fewer than 30%, fewer than 25%, fewer than 20%, fewer than 15%, fewer than 14%, fewer than 13%, fewer than 12%, fewer than 10%, or fewer than 5% of the nucleosides in the second modified oligonucleotide comprise a fluorine.

Embodiment 84. The oligomeric duplex of any one of embodiments 1-82, wherein no more than 4 nucleosides, no more than 3 nucleosides, no more than 2 nucleosides, or no more than 1 nucleoside in the second modified oligonucleotide comprise a modified sugar moiety comprising a fluorine or a sugar surrogate comprising a fluorine; and wherein optionally none of the nucleosides before the seventh or after the eleventh nucleoside counting from the 5′ end of the second modified oligonucleotide comprise a modified sugar moiety comprising a fluorine or a sugar surrogate comprising a fluorine.

Embodiment 85. The oligomeric duplex of any one of embodiments 1-84, wherein one or more of the seventh, ninth and eleventh nucleosides counting from the 5′ end of the second modified oligonucleotide comprises a modified sugar moiety comprising a fluorine.

Embodiment 86. The oligomeric duplex of embodiment 85, wherein two or more of the seventh, ninth and eleventh nucleosides counting from the 5′ end of the second modified oligonucleotide comprise a modified sugar moiety comprising a fluorine.

Embodiment 87. The oligomeric duplex of embodiment 85, wherein the seventh, ninth and eleventh nucleosides counting from the 5′ end of the second modified oligonucleotide comprise a modified sugar moiety comprising a fluorine.

Embodiment 88. The oligomeric duplex of any one of embodiments 1-84, wherein one or both of the tenth and eleventh nucleosides counting from the 5′ end of the second modified oligonucleotide comprises a modified sugar moiety comprising a fluorine.

Embodiment 89. The oligomeric duplex of any one of embodiments 84-88, wherein the modified sugar moiety comprising a fluorine is a 2′-fluoro sugar moiety.

Embodiment 90. The oligomeric duplex of any one of embodiments 1-89, wherein fewer than 30%, fewer than 25%, fewer than 20%, fewer than 15%, fewer than 14%, fewer than 13%, fewer than 12%, fewer than 10%, fewer than 5%, or fewer than 1% of the total nucleosides in the oligomeric duplex comprise a fluorine.

Embodiment 91. The oligomeric duplex of any one of embodiments 1-90, wherein one or more of the nucleosides of the second modified oligonucleotide independently comprises a sugar surrogate or a bicyclic sugar moiety.

Embodiment 92. The oligomeric duplex of embodiment 91, wherein the bicyclic sugar moiety comprises a 2′-4′ bridge selected from —O—CH₂—; and —O—CH(CH₃)—.

Embodiment 93. The oligomeric duplex of any one of embodiments 1-92, wherein one or more of the nucleosides of the second modified oligonucleotide comprises a 2′-OMe sugar moiety.

Embodiment 94. The oligomeric duplex of embodiment 93, wherein at least 10%, at least 20%, at least 30%, at least 40%, at least 50%, at least 60%, at least 65%, at least 70%, at least 80%, at least 85%, or at least 90% of the nucleosides of the second modified oligonucleotide comprise a 2′-OMe sugar moiety.

Embodiment 95. The oligomeric duplex of embodiment 93 or embodiment 94, wherein the one or more nucleosides comprising a 2′-OMe sugar moiety are in a region of the sequence of the second modified oligonucleotide between and including the third and nineteenth nucleosides counting from the 5′ end of the second modified oligonucleotide.

Embodiment 96. The oligomeric duplex of embodiment 93 or embodiment 94, wherein the 3′-terminal nucleoside of the second modified oligonucleotide comprises a 2′-OMe sugar moiety and/or the nucleoside immediately 5′ of the 3′-terminal nucleoside of the second modified oligonucleotide comprises a 2′-OMe sugar moiety.

Embodiment 97. The oligomeric duplex of any one of embodiments 93, 94, and 96, wherein the 5′-terminal nucleoside of the second modified oligonucleotide comprises a 2′-OMe sugar moiety and/or the nucleoside immediately 3′ of the 5′-terminal nucleoside of the second modified oligonucleotide comprises a 2′-OMe sugar moiety.

Embodiment 98. The oligomeric duplex of embodiment 93 or embodiment 94, wherein the 5′-terminal nucleoside, the nucleoside immediately 3′ of the 5′-terminal nucleoside, the 3′-terminal nucleoside, and the nucleoside immediately 5′ of the 3′-terminal nucleoside of the second modified oligonucleotide comprises a 2′-OMe sugar moiety.

Embodiment 99. The oligomeric duplex of any one of embodiments 1-98, wherein one or more of the nucleosides of the second modified oligonucleotide comprise a 2′-MOE sugar moiety.

Embodiment 100. The oligomeric duplex of any one of embodiments 1-95, wherein the 3′-terminal nucleoside of the second modified oligonucleotide comprises a 2′-MOE sugar moiety and/or the nucleoside immediately 5′ of the 3′-terminal nucleoside of the second modified oligonucleotide comprises a 2′-MOE sugar moiety.

Embodiment 101. The oligomeric duplex of any one of embodiments 1-96, 99 and 100, wherein the 5′-terminal nucleoside of the second modified oligonucleotide comprises a 2′-MOE sugar moiety and/or the nucleoside immediately 3′ of the 5′-terminal nucleoside of the second modified oligonucleotide comprises a 2′-MOE sugar moiety.

Embodiment 102. The oligomeric duplex of any one of embodiments 1-95, wherein the 5′-terminal nucleoside, the nucleoside immediately 3′ of the 5′-terminal nucleoside, the 3′-terminal nucleoside, and the nucleoside immediately 5′ of the 3′-terminal nucleoside of the second modified oligonucleotide comprises a 2′-MOE sugar moiety.

Embodiment 103. The oligomeric duplex of any one of embodiments 1-102, wherein the second modified oligonucleotide has a sugar motif (5′ to 3′) selected from among: eeyyyyfyfyfyyyyyyyyee, eeyyyyfyfyfyyyyyyyyyy, yyyyyyfyfyfyyyyyyyyee, yyyyyyfyfyfyyyyyyyyyy, eeyyyyfyfffyyyyyyyyee, eeyyyyfyfffyyyyyyyyyy, yyyyyyfyfffyyyyyyyyee, yyyyyyfyfffyyyyyyyyyy, eeyyyyyyyffyyyyyyyyee, eeyyyyyyyffyyyyyyyyyy, yyyyyyyyyffyyyyyyyyee, yyyyyyyyyffyyyyyyyyyy, eeyyyyyffyyyyyyyyee, eeyyyyyffyyyyyyyyyy, yyyyyyyffyyyyyyyyee, yyyyyyyffyyyyyyyyyy, yyyyyyfyfffyyyyyyyy, eeyyyyfyfffyyyyyyee, eeyyyyfyfffyyyyyyyy, yyyyyyfyfffyyyyyyee, eeyyfyfffyyyyyyyyee, eeyyfyfffyyyyyyyyyy, yyyyfyfffyyyyyyyyee, yyyyfyfffyyyyyyyyyy, eeyyyyyyyydyyyyyyyyee, eeyyyyyyyydyyyyyyyyyy, yyyyyyyyyydyyyyyyyyee, yyyyyyyyyydyyyyyyyyyy, eeyyyyyyyyyyyyyyyyyee, eeyyyyyyyyyyyyyyyyyyy, yyyyyyyyyyyyyyyyyyyee, and yyyyyyyyyyyyyyyyyyyyy wherein each “y” represents a 2′-OMe sugar moiety, each “e” represents a 2′-MOE sugar moiety, each “f” represents a 2′-fluoro sugar moiety, and each “d” represents a 2′-deoxy sugar moiety.

Embodiment 104. The oligomeric duplex of any one of embodiments 1-102, wherein the second modified oligonucleotide has a sugar motif (5′ to 3′) selected from among: eeyyyyyyyffyyyyyyyyee, eeyyyyfyfyfyyyyyyyyee, yyyyyyyyyffyyyyyyyyyy, and yyyyyyfyfyfyyyyyyyyyy, wherein each “y” represents a 2′-OMe sugar moiety, each “e” represents a 2′-MOE sugar moiety, and each “f” represents a 2′-fluoro sugar moiety.

Embodiment 105. The oligomeric duplex of any one of embodiments 1-104, wherein (1) the nucleobase of the 3′-terminal nucleoside and/or of the nucleoside immediately 5′ of the 3′-terminal nucleoside of the first modified oligonucleotide and/or of the second modified oligonucleotide is an adenine or thymine; and/or (2) the nucleobase of the 5′-terminal nucleoside of the first modified oligonucleotide is a thymine.

Embodiment 106. The oligomeric duplex of any one of embodiments 1-104, wherein the nucleobase of the 3′-terminal nucleoside and the nucleoside immediately 5′ of the 3′-terminal nucleoside of the first modified oligonucleotide is an adenine, and the nucleobase of the 5′-terminal nucleoside of the first modified oligonucleotide is a thymine; and wherein the nucleobase of the 3′-terminal nucleoside of the second modified oligonucleotide is an adenine and the nucleobase of the nucleoside immediately 5′ of the 3′-terminal nucleoside of the second modified oligonucleotide is a thymine or uracil.

Embodiment 107. The oligomeric duplex of any one of embodiments 1-106, wherein the second modified oligonucleotide comprises at least one modified internucleoside linkage.

Embodiment 108. The oligomeric duplex of embodiment 107, wherein at least one modified internucleoside linkage is a phosphorothioate or mesyl phosphoramidate internucleoside linkage.

Embodiment 109. The oligomeric duplex of embodiment 107, wherein fewer than 50%, fewer than 40%, fewer than 30%, fewer than 20%, or fewer than 10% of the internucleoside linkages of the second modified oligonucleotide are modified internucleoside linkages.

Embodiment 110. The oligomeric duplex of any one of embodiments 107-109, wherein each internucleoside linkage of the second modified oligonucleotide is independently selected from a phosphodiester internucleoside linkage and a phosphorothioate internucleoside linkage.

Embodiment 111. The oligomeric duplex of any one of embodiments 107-109, wherein the internucleoside linkages between the first and second nucleosides and the second and third nucleosides counting from the 5′ end of the second modified oligonucleotide are modified internucleoside linkages.

Embodiment 112. The oligomeric duplex of any one of embodiments 107-109 and 111, wherein the internucleoside linkages between the first and second nucleosides and the second and third nucleosides counting from the 3′ end of the second modified oligonucleotide are modified internucleoside linkages.

Embodiment 113. The oligomeric duplex of any one of embodiments 107-109, wherein the internucleoside linkages between the first and second nucleosides and the second and third nucleosides counting from the 5′ end of the second modified oligonucleotide, and the internucleoside linkages between the first and second nucleosides and the second and third nucleosides counting from the 3′ end of the second modified oligonucleotide are modified internucleoside linkages.

Embodiment 114. The oligomeric duplex of any one of embodiments 107-113, wherein the modified internucleoside linkages are phosphorothioate internucleoside linkages.

Embodiment 115. The oligomeric duplex of embodiment 114, wherein all other internucleoside linkages in the second modified oligonucleotide are phosphodiester internucleoside linkages.

Embodiment 116. The oligomeric duplex of embodiment 115, wherein the second modified oligonucleotide has an internucleoside linkage motif of ssooooooooooooooooss and a sugar motif (5′ to 3′) selected from among: eeyyyyyyyffyyyyyyyyee, eeyyyyfyfyfyyyyyyyyee, yyyyyyyyyffyyyyyyyyyy, and yyyyyyfyfyfyyyyyyyyyy; wherein each ““y” represents a 2′-OMe sugar moiety, each “e” represents a 2′-MOE sugar moiety, each “f” represents a 2′-fluoro sugar moiety, each “o” represents a phosphodiester internucleoside linkage and each “s” represents a phosphorothioate internucleoside linkage.

Embodiment 117. The oligomeric duplex of any one of embodiments 1-116, wherein the nucleobase sequence of the second modified oligonucleotide comprises at least 14, at least 15, at least 16, at least 17, at least 18, at least 19, at least 20, or at least 21 contiguous nucleobases of any one of the nucleobase sequences of SEQ ID NOs: 315-626, 783-938, 977-1016, 1027-1032, 1256-1257, or 1288-1290.

Embodiment 118. The oligomeric duplex of any one of embodiments 1-116, wherein the nucleobase sequence of the second modified oligonucleotide comprises the nucleobase sequence of any one of SEQ ID NOs: 315-626, 783-938, 977-1016, 1027-1032, 1256-1257, or 1288-1290.

Embodiment 119. The oligomeric duplex of any one of embodiments 1-116, wherein the nucleobase sequence of the second modified oligonucleotide consists of the nucleobase sequence of any one of SEQ ID NOs: 315-626, 783-938, 977-1016, 1027-1032, 1256-1257, or 1288-1290.

Embodiment 120. The oligomeric duplex of any one of embodiments 1-116, wherein the nucleobase sequence of the second modified oligonucleotide comprises at least 16 contiguous nucleobases of any one of the nucleobase sequences of SEQ ID NOs: 341, 483, 488, 1011-1016, 1027-1032, 1256-1257, or 1288-1290.

Embodiment 121. The oligomeric duplex of any one of embodiments 1-116, wherein the nucleobase sequence of the second modified oligonucleotide comprises the nucleobase sequence of any one of SEQ ID NOs: 341, 483, 488, 1011-1016, 1027-1032, 1256-1257, or 1288-1290.

Embodiment 122. The oligomeric duplex of any one of embodiments 1-116, wherein the nucleobase sequence of the second modified oligonucleotide consists of the nucleobase sequence of any one of SEQ ID NOs: 341, 483, 488, 1011-1016, 1027-1032, 1256-1257, or 1288-1290.

Embodiment 123. An oligomeric duplex comprising:
(1) a first oligomeric compound comprising a first modified oligonucleotide consisting of 18 to 30 contiguous linked nucleosides that is at least 80%, at least 85%, at least 90%, at least 95%, or at least 99% complementary, or 100% complementary to an equal length portion within nucleobases selected from any one of 295-316, 296-317, 297-318, 298-320, 299-320, 300-321, 301-322, 302-324, 303-324, 304-325, 304-326, 305-327, 306-325, 306-328, 307-328, 308-329, 309-330, 310-331, 311-332, 312-333, 313-334, 314-335, 315-336, 316-337, 317-339, 318-339, 319-340, 320-341, 321-342, 322-344, 323-345, 324-345, 324-346, 325-345, 325-347, 326-347, 327-348, 328-345, 328-349, 329-350, 330-351, 331-352, 332-353, 333-354, 334-355, 505-526, 506-528, 507-529, 508-530, 509-531, 510-531, 511-532, 512-533, 513-535, 514-535, 514-536, 515-536, 516-535, 516-537, 517-538, 518-539, 519-541, 520-541, 521-542, 522-544, 523-544, 524-546, 535-556, 536-557, 537-558, 538-559, 539-560, 540-561, 541-563, 542-563, 543-564, 544-565, 545-566, 546-567, 547-568, 548-570, 549-571, 550-572, 551-572, 552-574, 553-574, 554-575, 555-577, 556-578, 557-579, 558-580, 559-580, 560-581, 561-582, 562-583, 563-584, 595-616, 596-618, 597-618, 598-620, 599-621, 600-622, 601-623, 602-623, 603-624, 604-625, 605-627, 606-628, 607-628, 608-629, 609-630, 610-631, 611-632, 612-633, 613-635, 665-687, 666-687, 667-689, 668-689, 669-690, 670-691, 671-692, 672-694, 673-694, 674-695, 675-696, 676-697, 677-698, 678-700, 679-701, 680-702, 681-703, 682-703, 683-704, 684-705, 685-706, 686-708, 687-708, 688-709, 689-710, 690-711, 691-712, 692-714, 693-715, 1675-1696, 1676-1698, 1677-1698, 1678-1699, 1679-1700, 1680-1702, 1681-1702, 1682-1703, 1683-1705, 1684-1705, 1685-1706, 1686-1707, 1687-1709, 1688-1709, 1689-1710, 1690-1712, 1691-1713, 1692-1714, or 1693-1714 of SEQ ID NO: 1, and
(2) a second oligomeric compound comprising a second modified oligonucleotide consisting of 16 to 28 contiguous linked nucleosides that is at least 90% complementary to an equal length portion of the first modified oligonucleotide;
   wherein:
(a) each of the nucleosides of the first modified oligonucleotide and each of the nucleosides of the second modified oligonucleotide comprise a modified sugar moiety or a sugar surrogate,
(b) at least one of the modified sugar moiety or sugar surrogate of the first modified oligonucleotide comprises a fluorine, and
(c) fewer than 40% of the nucleosides of the first modified oligonucleotide comprise a fluorine.

Embodiment 124. An oligomeric duplex comprising, (1) a first oligomeric compound comprising a first modified oligonucleotide consisting of 18 to 30 contiguous linked nucleosides, wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases of any one of the nucleobase sequences of SEQ ID NOs: 3-314, 627-782, 939-976, 1033-1038, 1254-1255, or 1258-1287; and (2) a second oligomeric compound comprising a second modified oligonucleotide consisting of 16 to 28 contiguous linked nucleosides that is at least 90% complementary to an equal length portion of the first modified oligonucleotide; wherein: (a) each of the nucleosides of the first modified oligonucleotide and each of the nucleosides of the second modified oligonucleotide comprise a modified sugar moiety or a sugar surrogate, (b) at least one of the modified sugar moiety or sugar surrogate of the first modified oligonucleotide comprises a fluorine, and (c) fewer than 40% of the nucleosides of the first modified oligonucleotide comprise a fluorine.

Embodiment 125. The oligomeric duplex of embodiment 124, wherein each nucleoside of the oligomeric duplex comprises (i) a modified sugar moiety or (ii) a sugar moiety independently selected from a 2′-F sugar moiety, a 2′-MOE sugar moiety, a 2′-OMe sugar moiety, a 2′-deoxy sugar moiety, and a 3′-fluoro-hexitol sugar moiety.

Embodiment 126. The oligomeric duplex of embodiment 124, wherein the nucleobase sequence of the first modified oligonucleotide comprises or consists of the nucleobase sequence of any one of SEQ ID NOs: 3-314, 627-782, 939-976, 1033-1038, 1254-1255, or 1258-1287.

Embodiment 127. The oligomeric duplex of embodiment 124, wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases of any one of the nucleobase sequences of SEQ ID NOs: 29,171,176, 967, 968, 971, 972, 974, 975, 1033-1038, 1254-1255, or 1258-1287.

Embodiment 128. The oligomeric duplex of embodiment 124, wherein the nucleobase sequence of the first modified oligonucleotide comprises the nucleobase sequence of any one of SEQ ID NOs: 29, 171, 176, 967, 968, 971, 972, 974, 975, 1033-1038, 1254-1255, or 1258-1287.

Embodiment 129. The oligomeric duplex of embodiment 124, wherein the nucleobase sequence of the first modified oligonucleotide consists of the nucleobase sequence of any one of SEQ ID NOs: 29, 171, 176, 967, 968, 971, 972, 974, 975, 1033-1038, 1254-1255, or 1258-1287.

Embodiment 130. The oligomeric duplex of embodiment 124, wherein the nucleobase sequence of the second modified oligonucleotide comprises least 8, at least 9, at least 10, at least 11, at least 12, at least 13, at least 14, at least 15, at least 16, at least 17, at least 18, at least 19, at least 20, or at least 21 contiguous nucleobases of any one of the nucleobase sequences of SEQ ID NOs: 315-626, 783-938, 977-1016, 1027-1032, 1256-1257, or 1288-1290.

Embodiment 131. The oligomeric duplex of embodiment 126, wherein the nucleobase sequence of the second modified oligonucleotide comprises the nucleobase sequence of any one of SEQ ID NOs: 315-626, 783-938, 977-1016, 1027-1032, 1256-1257, or 1288-1290.

Embodiment 132. The oligomeric duplex of embodiment 126, wherein the nucleobase sequence of the second modified oligonucleotide consists of the nucleobase sequence of any one of SEQ ID NOs: 315-626, 783-938, 977-1016, 1027-1032, 1256-1257, or 1288-1290.

Embodiment 133. The oligomeric duplex of embodiment 127, wherein the nucleobase sequence of the second modified oligonucleotide comprises at least 8, at least 9, at least 10, at least 11, at least 12, at least 13, at least 14, at least 15, at least 16, at least 17, at least 18, at least 19, at least 20, or at least 21 contiguous nucleobases of any one of the nucleobase sequences of SEQ ID NOs: 341, 483, 488, 1011-1016, 1027-1032, 1256-1257, or 1288-1290.

Embodiment 134. The oligomeric duplex of embodiment 128, wherein the nucleobase sequence of the second modified oligonucleotide comprises the nucleobase sequence of any one of SEQ ID NOs: 341, 483, 488, 1011-1016, 1027-1032, 1256-1257, or 1288-1290.

Embodiment 135. The oligomeric duplex of embodiment 129, wherein the nucleobase sequence of the second modified oligonucleotide consists of the nucleobase sequence of any one of SEQ ID NOs: 341, 483, 488, 1011-1016, 1027-1032, 1256-1257, or 1288-1290.

Embodiment 136. The oligomeric duplex of any one of embodiments 123-135, wherein (1) the nucleobase of the 3′-terminal nucleoside and/or of the nucleoside immediately 5′ of the 3′-terminal nucleoside of the first modified oligonucleotide and/or of the second modified oligonucleotide is an adenine or thymine; and/or (2) the nucleobase of the 5′-terminal nucleoside of the first modified oligonucleotide is a thymine.

Embodiment 137. The oligomeric duplex of any one of embodiments 123-135, wherein the nucleobase of the 3′-terminal nucleoside and the nucleoside immediately 5′ of the 3′-terminal nucleoside of the first modified oligonucleotide is an adenine, and the nucleobase of the 5′-terminal nucleoside of the first modified oligonucleotide is a thymine; and wherein the nucleobase of the 3′-terminal nucleoside of the second modified oligonucleotide is an adenine and the nucleobase of the nucleoside immediately 5′ of the 3′-terminal nucleoside of the second modified oligonucleotide is a thymine or uracil.

Embodiment 138. The oligomeric duplex of any one of embodiments 123-135, wherein the first oligomeric compound comprises a two-nucleoside overhang comprising the 3′-terminal nucleoside and the nucleoside immediately 5′ of the 3′-terminal nucleoside of the first modified oligonucleotide.

Embodiment 139. The oligomeric duplex of embodiment 138, wherein the overhang nucleosides are two modified adenosine (AA) nucleosides, two modified uridine (UU) nucleosides, two modified inosine (II) nucleosides, or two modified nucleosides wherein one is inosine and one is adenosine (AI or IA).

Embodiment 140. The oligomeric duplex of any one of embodiments 123-139, wherein none of the modified sugar moieties or sugar surrogates of the nucleosides of the second modified oligonucleotide comprises a fluorine.

Embodiment 141. The oligomeric duplex of any one of embodiments 123-139, wherein at least one of the modified sugar moieties or sugar surrogates of the second modified oligonucleotide comprises a fluorine and wherein fewer than 40%, fewer than 30%, fewer than 25%, fewer than 20%, fewer than 15%, fewer than 14%, fewer than 13%, fewer than 12%, fewer than 10%, or fewer than 5% of the nucleosides of the second modified oligonucleotide comprises a fluorine; and/or wherein fewer than 30%, fewer than 25%, fewer than 20%, fewer than 15%, fewer than 14%, fewer than 13%, fewer than 12%, fewer than 10%, or fewer than 5% of the total nucleosides in the oligomeric duplex comprise a fluorine.

Embodiment 142. The oligomeric duplex of any one of embodiments 123-139, wherein two, three or four of the nucleosides of the second modified oligonucleotide independently comprise a modified sugar moiety comprising a fluorine or a sugar surrogate comprising a fluorine or no more than 4 nucleosides, no more than 3 nucleosides, no more than 2 nucleosides, or no more than 1 nucleoside of the second modified oligonucleotide comprise a modified sugar moiety comprising a fluorine or a sugar surrogate comprising a fluorine.

Embodiment 143. The oligomeric duplex of any one of embodiments 123-142, wherein two, three or four of the nucleosides of the first modified oligonucleotide independently comprise a modified sugar moiety comprising a fluorine or a sugar surrogate comprising a fluorine or no more than 4 nucleosides, no more than 3 nucleosides, no more than 2 nucleosides, or no more than 1 nucleoside of the first modified oligonucleotide comprise a modified sugar moiety comprising a fluorine or a sugar surrogate comprising a fluorine.

Embodiment 144. The oligomeric duplex of any one of embodiments 123-143, wherein no more than 1 nucleoside, no more than 2 nucleosides, or no more than 3 nucleosides within the sequence of the first modified oligonucleotide between and including the third and twenty-first nucleosides counting from the 5′ end of the modified oligonucleotide comprise a modified sugar moiety comprising a fluorine or a sugar surrogate comprising a fluorine.

Embodiment 145. The oligomeric duplex of any one of embodiments 123-144, wherein one or more nucleoside comprising a modified sugar moiety comprising a fluorine or a sugar surrogate comprising a fluorine of the first modified oligonucleotide is/are independently selected from:
i. the second nucleoside counting from the 5′ end,
ii. the fourteenth and sixteenth nucleosides counting from the 5′ end, or
iii. the second, fourteenth and sixteenth nucleosides counting from the 5′ end;
wherein each modified sugar moiety comprising a fluorine or sugar surrogate comprising a fluorine is independently a 2′-fluoro sugar moiety or a 3′-fluoro-hexitol sugar moiety.

Embodiment 146. The oligomeric duplex of any one of embodiments 123-145, wherein none of the nucleosides of the second modified oligonucleotide is a 2′-deoxynucleoside and/or none of the nucleosides of the first modified oligonucleotide is a 2′-deoxynucleoside.

Embodiment 147. The oligomeric duplex of any one of embodiments 123-146, wherein all of the nucleosides of the second modified oligonucleotide comprise a modified ribosyl sugar moiety.

Embodiment 148. The oligomeric duplex of any one of embodiments 123-145 and 147, wherein one, two, or three of the nucleosides of the first modified oligonucleotide is/are a 2′-deoxynucleoside or wherein no more than 3, nor more than 2, or no more than one nucleoside(s) of the first modified oligonucleotide is/are a 2′-deoxynucleoside(s), or wherein only 3, only 2, or only one nucleoside(s) of the first modified oligonucleotide is/are a 2′-deoxynucleoside(s).

Embodiment 149. The oligomeric duplex of embodiment 148, wherein the fifth and seventh nucleosides counting from the 5′ end of the first modified oligonucleotide are 2′-deoxynucleosides, or wherein the sixth nucleoside counting from the 5′ end of the first modified oligonucleotide is a 2′-deoxynucleoside.

Embodiment 150. The oligomeric duplex of any one of embodiments 123-149, wherein:
(a) the 5′- and 3′-terminal nucleosides, the nucleoside immediately 3′ of the 5′-terminal nucleoside, and the nucleoside immediately 5′ of the 3′-terminal nucleoside of the second modified oligonucleotide comprise a 2′-MOE sugar moiety, and/or
(b) the 5′- and 3′-terminal nucleosides and the nucleoside immediately 5′ of the 3′-terminal nucleoside of the first modified oligonucleotide comprise a 2′-MOE sugar moiety, and/or
(c) any nucleoside in the first and second modified oligonucleotides that does not comprise a fluorine or a 2′-MOE sugar moiety comprises a 2′-OMe sugar moiety.

Embodiment 151. The oligomeric duplex of any one of embodiments 123-149, wherein the 3′-terminal nucleoside of the first modified oligonucleotide comprises a 2′-OMe sugar moiety and/or the nucleoside immediately 5′ of the 3′-terminal nucleoside of the first modified oligonucleotide comprises a 2′—OMe sugar moiety and optionally the 5′-terminal nucleoside of the first modified oligonucleotide comprises a 2′-MOE sugar moiety.

Embodiment 152. The oligomeric duplex of any one of embodiments 123-151, wherein the first oligomeric compound comprises a stabilized phosphate group attached to the 5′-terminal nucleoside of the first modified oligonucleotide.

Embodiment 153. The oligomeric duplex of embodiment 152, wherein the stabilized phosphate group comprises a cyclopropyl phosphonate or a vinyl phosphonate.

Embodiment 154. The oligomeric duplex of any one of embodiments 123-153, wherein no more than four of the nucleosides of the first modified oligonucleotide comprise a modified sugar moiety comprising a fluorine or a sugar surrogate comprising a fluorine.

Embodiment 155. The oligomeric duplex of embodiment 154, wherein the first modified oligonucleotide has a sugar motif (5′ to 3′) selected from among: efyyyfyyyyyyyfyfyyyyyee, efyyyfyyyyyyyfyfyyyyyyy, e[FHNA]yyyfyyyyyyyfyfyyyyyee, e[FHNA]yyyfyyyyyyyfyfyyyyyyy, efyyyfyyyyyyyfyfyyyyyey, e[FHNA]yydydyyyyfyfyfyyyyyyy and efyyyfyyyyyyyfyfyyyee; wherein each “d” represents a 2′-deoxy sugar moiety, each “y” represents a 2′-OMe sugar moiety, each “e” represents a 2′-MOE sugar moiety, each “f” represents a 2′-fluoro sugar moiety, and each “[FHNA]” represents a 3′-fluoro-hexitol sugar moiety.

Embodiment 156. The oligomeric duplex of any one of embodiments 123-153, wherein no more than three of the nucleosides of the first modified oligonucleotide comprise a modified sugar moiety comprising a fluorine or a sugar surrogate comprising a fluorine.

Embodiment 157. The oligomeric duplex of embodiment 156, wherein the first modified oligonucleotide has a sugar motif (5′ to 3′) selected from among: efyyydyyyyyyyfyfyyyyyee, efyyydyyyyyyyfyfyyyyyyy, e[FHNA]yyydyyyyyyyfyfyyyyyee, e[FHNA]yyydyyyyyyyfyfyyyyyyy, efyydydyyyyyyfyfyyyyyee, efyydydyyyyyyfyfyyyyyyy, e[FHNA]yydydyyyyyyfyfyyyyyee, e[FHNA]yydydyyyyyyfyfyyyyyyy, efyyyyyyyyyyyfyfyyyyee, efyyyfyyyyyyyfyyyyyyyyy, efyyyfyyyyyyyfydyyyyyee, efyyyfyyyyyyyfydyyyyyyy, efyyyfyyyyyyyfydyyyyyee, efyyyfyyyyyyyfydyyyyyyy, efyydydyyyyyyfyfyyyyye, efyydydyyyyyyfyfyyyyyy, efyyydyyyyyyyfyfyyyyye, efyyydyyyyyyyfyfyyyyyy, efyyyyyyyyyyyfyfyyyyye, efyyyyyyyyyyyfyfyyyyyy, efyydydyyyyyyfyfyyyyy, efyyydyyyyyyyfyfyyyyy, and efyyyyyyyyyyyfyfyyyyy; wherein each “d” represents a 2′-deoxy sugar moiety, each “y” represents a 2′-OMe sugar moiety, each “e” represents a 2′-MOE sugar moiety, each “f” represents a 2′-fluoro sugar moiety, and each “[FHNA]” represents a 3′-fluoro-hexitol sugar moiety.

Embodiment 158. The oligomeric duplex of any one of embodiments 123-153, wherein no more than two of the nucleosides of the first modified oligonucleotide comprise a modified sugar moiety comprising a fluorine or a sugar surrogate comprising a fluorine.

Embodiment 159. The oligomeric duplex of embodiment 158, wherein the first modified oligonucleotide has a sugar motif (5′ to 3′) selected from among: efyyyyyyyyyyyfyyyyyyyee, efyyyyyyyyyyyfyyyyyyyyy, e[FHNA]yyyyyyyyyyy[FHNA]yyyyyyyee, and e[FHNA]yyyyyyyyyyy[FHNA]yyyyyyyyy; wherein each “y” represents a 2′-OMe sugar moiety, each “e” represents a 2′-MOE sugar moiety, each “f” represents a 2′-fluoro sugar moiety, and each “[FHNA]” represents a 3′-fluoro-hexitol sugar moiety.

Embodiment 160. The oligomeric duplex of any one of embodiments 123-153, wherein only one of the nucleosides of the first modified oligonucleotide comprises a modified sugar moiety comprising a fluorine or a sugar surrogate comprising a fluorine.

Embodiment 161. The oligomeric duplex of embodiment 160, wherein the first modified oligonucleotide has a sugar motif (5′ to 3′) selected from among: efyyydyyyyyyydydyyyyyyy, efyyydyyyyyyydydyyyyyee, e[FHNA]yyydyyyyyyydydyyyyyyy, and e[FHNA]yyydyyyyyyydydyyyyyee; wherein each “d” represents a 2′-deoxy sugar moiety, each “y” represents a 2′-OMe sugar moiety, each “e” represents a 2′-MOE sugar moiety, each “f” represents a 2′-fluoro sugar moiety, and each “[FHNA]” represents a 3′-fluoro-hexitol sugar moiety.

Embodiment 162. The oligomeric duplex of any one of embodiments 123-158 and 160, wherein no more than two of the nucleosides of the first modified oligonucleotide are 2′-deoxynucleosides.

Embodiment 163. The oligomeric duplex of embodiment 162 wherein the first modified oligonucleotide has a sugar motif (5′ to 3′) selected from among: efyydydyyyyyyfyfyyyyyee, efyydydyyyyyyfyfyyyyyyy, e[FHNA]yydydyyyyyyfyfyyyyyee, e[FHNA]yydydyyyyyyfyfyyyyyyy, efyydydyyyyyyfyfyyyyye, efyydydyyyyyyfyfyyyyyy, and efyydydyyyyyyfyfyyyyy; wherein each “d” represents a 2′-deoxy sugar moiety, each “y” represents a 2′-OMe sugar moiety, each “e” represents a 2′-MOE sugar moiety, each “f” represents a 2′-fluoro sugar moiety, and each “[FHNA]” represents a 3′-fluoro-hexitol sugar moiety.

Embodiment 164. The oligomeric duplex of any one of embodiments 123-154, 156, 158 and 160, wherein only one of the nucleosides of the first modified oligonucleotide is a 2′-deoxynucleoside.

Embodiment 165. The oligomeric duplex of embodiment 164, wherein the first modified oligonucleotide has a sugar motif (5′ to 3′) selected from among: efyyydyyyyyyyfyfyyyyyee, efyyydyyyyyyyfyfyyyyyyy, e[FHNA]yyydyyyyyyyfyfyyyyyee, e[FHNA]yyydyyyyyyyfyfyyyyyyy, eyyyfyyyyyyyfydyyyyyyy, efyyyfyyyyyyyfydyyyyyee, eyyy[FHNA]yyyyyyyfydyyyyyyy, e[FHNA]yyyfyyyyyyyfydyyyyyee, efyyydyyyyyyyfyfyyyyye, efyyydyyyyyyyfyfyyyyyy, and efyyydyyyyyyyfyfyyyyy; wherein each “d” represents a 2′-deoxy sugar moiety, each “y” represents a 2′-OMe sugar moiety, each “e” represents a 2′-MOE sugar moiety, each “f” represents a 2′-fluoro sugar moiety, and each “[FHNA]” represents a 3′-fluoro-hexitol sugar moiety.

Embodiment 165a. The oligomeric duplex of embodiment 164, wherein the first modified oligonucleotide has a sugar motif (5′ to 3′) selected from among: efyyydyyyyyyyfyfyyyyyee, efyyydyyyyyyyfyfyyyyyyy, e[FHNA]yyydyyyyyyyfyfyyyyyee, e[FHNA]yyydyyyyyyyfyfyyyyyyy, eyyyfyyyyyyyfydyyyyyyy, efyyyfyyyyyyyfydyyyyyee, efyyy[FHNA]yyyyyyyfydyyyyyyy, e[FHNA]yyyfyyyyyyyfydyyyyyee, efyyydyyyyyyyfyfyyyyye, efyyydyyyyyyyfyfyyyyyy, and efyyydyyyyyyyfyfyyyyy; wherein each “d” represents a 2′-deoxy sugar moiety, each “y” represents a 2′-OMe sugar moiety, each “e” represents a 2′-MOE sugar moiety, each “f” represents a 2′-fluoro sugar moiety, and each “[FHNA]” represents a 3′-fluoro-hexitol sugar moiety.

Embodiment 166. The oligomeric duplex of any one of embodiments 123-154, wherein none of the nucleosides of the first modified oligonucleotide comprises a 2′-deoxy sugar moiety.

Embodiment 167. The oligomeric duplex of embodiment 166, wherein the first modified oligonucleotide has a sugar motif (5′ to 3′) selected from among: efyyyyyyyyyyyfyfyyyyyee, efyyyfyyyyyyyfyyyyyyyyy, efyyyyyyyyyyyfyfyyyyye, efyyyyyyyyyyyfyfyyyyyy, and efyyyyyyyyyyyfyfyyyyy, wherein each “y” represents a 2′-OMe sugar moiety, each “e” represents a 2′-MOE sugar moiety, and each “f” represents a 2′-fluoro sugar moiety.

Embodiment 168. The oligomeric duplex of any one of embodiments 123-139 and 141-167, wherein no more than four of the nucleosides of the second modified oligonucleotide comprise a modified sugar moiety comprising a fluorine or a sugar surrogate comprising a fluorine.

Embodiment 169. The oligomeric duplex of embodiment 168, wherein the second modified oligonucleotide has a sugar motif (5′ to 3′) selected from among: yyyyyyfyfffyyyyyyyyyy, eeyyyyfyfffyyyyyyyyee, yyyyyyfyfffyyyyyyyy, eeyyyyfyfffyyyyyyee, yyyyfyfffyyyyyyyyyy, and eeyyfyfffyyyyyyyyee; wherein each “y” represents a 2′-OMe sugar moiety, each “e” represents a 2′-MOE sugar moiety, and each “f” represents a 2′-fluoro sugar moiety.

Embodiment 170. The oligomeric duplex of any one of embodiments 123-139 and 141-167, wherein no more than three of the nucleosides of the second modified oligonucleotide comprise a modified sugar moiety comprising a fluorine or a sugar surrogate comprising a fluorine.

Embodiment 171. The oligomeric duplex of embodiment 170, wherein the second modified oligonucleotide has a sugar motif (5′ to 3′) selected from among: eeyyyyfyfyfyyyyyyyyee and yyyyyyfyfyfyyyyyyyyyy; wherein each “y” represents a 2′-OMe sugar moiety, each “e” represents a 2′-MOE sugar moiety, and each “f” represents a 2′-fluoro sugar moiety.

Embodiment 172. The oligomeric duplex of any one of embodiments 123-139 and 141-167, wherein no more than two of the nucleosides of the second modified oligonucleotide comprise a modified sugar moiety comprising a fluorine or a sugar surrogate comprising a fluorine.

Embodiment 173. The oligomeric duplex of embodiment 172, wherein the second modified oligonucleotide has a sugar motif (5′ to 3′) selected from among: eeyyyyyyyffyyyyyyyyee, yyyyyyyyyffyyyyyyyyyy, eeyyyyyffyyyyyyyyee, and yyyyyyyffyyyyyyyyyy; wherein each “y” represents a 2′-OMe sugar moiety, each “e” represents a 2′-MOE sugar moiety, and each “f” represents a 2′-fluoro sugar moiety.

Embodiment 174. The oligomeric duplex of any one of embodiments 123-167, wherein none of the nucleosides of the second modified oligonucleotide comprises a modified sugar moiety comprising a fluorine or a sugar surrogate comprising a fluorine and wherein the second modified oligonucleotide has a sugar motif (5′ to 3′) selected from among: yyyyyyyyyyyyyyyyyyyyy, eeyyyyyyyyyyyyyyyyyee, yyyyyyyyyydyyyyyyyyyy, and eeyyyyyyyydyyyyyyyyee; wherein each “y” represents a 2′-OMe sugar moiety, each “e” represents a 2′-MOE sugar moiety, and each “d” represents a 2′-deoxy sugar moiety.

Embodiment 175. The oligomeric duplex of any one of embodiments 123-174, wherein one or more of the internucleoside linkages of the first modified oligonucleotide is/are a modified internucleoside linkage and/or wherein fewer than 50%, fewer than 40%, fewer than 30%, fewer than 20%, or fewer than 10% of the internucleoside linkages of the first modified oligonucleotide are modified internucleoside linkages.

Embodiment 176. The oligomeric duplex of any one of embodiments 123-175, wherein the internucleoside linkages between the first and second nucleosides and the second and third nucleosides counting from the 5′ end of the first modified oligonucleotide, and the internucleoside linkages between the first and second nucleosides and the second and third nucleosides counting from the 3′ end of the first modified oligonucleotide are phosphorothioate internucleoside linkages.

Embodiment 177. The oligomeric duplex of any one of embodiments 123-175, wherein the first modified oligonucleotide has an internucleoside linkage motif (5′ to 3′) selected from among: ssooooooooooooooooooss, ssooosooooooooooooooss, ssoososoooooooooooooss, ssoooooooooooooooooos, ssoooooooooooooooooo, and ssooooooooooooooooss; wherein each “o” represents a phosphodiester internucleoside linkage and each “s” represents a phosphorothioate internucleoside linkage.

Embodiment 178. The oligomeric duplex of any one of embodiments 123-177, wherein one or more of the internucleoside linkages of the second modified oligonucleotide is/are a modified internucleoside linkage(s).

Embodiment 179. The oligomeric duplex of any one of embodiments 123-178, wherein fewer than 50%, fewer than 40%, fewer than 30%, fewer than 20%, or fewer than 10% of the internucleoside linkages of the second modified oligonucleotide are modified internucleoside linkages.

Embodiment 180. The oligomeric duplex of any one of embodiments 123-179, wherein the internucleoside linkages between the first and second nucleosides and the second and third nucleosides counting from the 5′ end of the second modified oligonucleotide, and the internucleoside linkages between the first and second nucleosides and the second and third nucleosides counting from the 3′ end of the second modified oligonucleotide are phosphorothioate internucleoside linkages.

Embodiment 181. The oligomeric duplex of any one of embodiments 123-180, wherein the second modified oligonucleotide has an internucleoside linkage motif (5′ to 3′) selected from among: ssooooooooooooooooss, ssooooooosooooooooss, ssoooooozozoooooooss, and ssooooooooooooooss; wherein each “o” represents a phosphodiester internucleoside linkage, each “s” represents a phosphorothioate internucleoside linkage, and each ‘z’ represents a mesyl phosphoramidate internucleoside linkage.

Embodiment 182. The oligomeric duplex of any one of embodiments 1-4, 12, 18, 19, 30-69, 72, 80, 105-115, 117-139, 152, 153, and 175-181, wherein the first modified oligonucleotide has a first sugar motif (1^{st}) and the second modified oligonucleotide has a second sugar motif (2^{nd}) and the first and second sugar motifs are selected from among the following combinations (5′ to 3′): 1^{st}: efyydydyyyyyyfyfyyyyyee and 2^{nd}: eeyyyyfyfyfyyyyyyyyee; 1^{st}: efyydydyyyyyyfyfyyyyyyy and 2^{nd}: yyyyyfyyyyyyy; 1^{st} efyydydyyyyyyfyfyyyyyee and 2^{nd}: yyyyyyjfffyyyyyyyyyy; 1^{st}: efyydydyyyyyyfyfyyyyyyy and 2^{nd}: eeyyyyfyfyfyyyyyyyyee; 1^{st}: efyyyyyyyyyyyfyfyyyyyee and 2^{nd}: eeyyy ffyyyyyyyyee; 1^{st}: efyyyyyyyyyyyfyfyyyyyyy and 2^{nd}: yyyyyyyyyffyyyyyyyyyy; 1^{st}: efyyyyyyyyyyyfyfyyyyyee and 2^{nd}: yyyyyyyyyffyyyyyyyyyy; 1^{st}: efyyyyyyyyyyyfyfyyyyyyy and 2^{nd}: eeyyyyyyyffyyyyyyyyee; 1^{st}: efyyydyyyyyyyfyfyyyyyee and 2^{nd}: eeyyyyyyyffyyyyyyyyee; 1^{st}: efyyydyyyyyyyfyfyyyyyyy and 2^{nd}: yyyyyyyyyffyyyyyyyyyy; 1^{st}: efyyydyyyyyyyfyfyyyyyee and 2^{nd}: YyyyyyyyffyYyyyyyyy; 1^{st}: efyyydyyyyyyyfyfyyyyyyy and 2^{nd}: eeyyyyyyyffyyyyyyyyee; 1^{st}: efyydydyyyyyyfyfyyyyyee and 2^{nd}: eeyyyyyyyffyyyyyyyyee; 1^{st}: efyydydyyyyyyfyfyyyyyyy and 2^{nd}: Yyyyyyyyyffyyyyyyyyyy; 1^{st}: efyydydyyyyyyfyfyyyyyee and 2^{nd}: yy=yyyffyy=yyyy; 1^{st}: efyydydyyyyyyfyfyyyyyyy and 2^{nd}: eeyyyyyyyffyyyyyyyyee; 1^{st}: efyyydyyyyyyyfyfyyyyyee and 2^{nd}: eeyyyyfyfyfyyyyyyyyee; 1^{st}: efyyydyyyyyyyfyfyyyyyyy and 2^{nd}: yyyyyfyjffyyyyyyyyyy; 1^{st}: efyyydyyyyyyyfyfyyyyyee and 2^{nd}: Yyyyyyfyfyfyyyyyyyyyy; 1^{st}: efyyydyyyyyyyfyfyyyyyyy and 2^{nd}: eeyyyyfyfyfyyyyyyyyee; 1^{st}: efyyyyyyyyyyyfyfyyyyyee and 2^{nd}: eeyyyyfyfyfyyyyee; 1^{st}: efyyyyyyyyyyfyfyyyyyyy and 2^{nd}: yyyyyyfyfyfyyyyyyyyyy; 1^{st}: efyyyyyyyyyyyfyfyyyyyee and 2^{nd}: yyyyyyfyfyfyyyyyyyyyy 1^{st}. efyyyyyyyyyyyfyfyyyyyyy and 2^{nd}: eeyyyyfyfyfyyYYYYYYee; 1^{st}: efyyyyyyyyyyyfyfyyyyye and 2^{nd}: eeyyyyyyyffyyyyyyyyee; 1^{st}: efyyyyyyyyyyyfyfyyyyyy and 2^{nd}: yyyyyyyyyffyyyyyyyyy; 1^{st}: efyyyyyyyyyyyfyfyyyyye and 2^{nd}: yyyyyyyffyyyyyyyyyy; 1^{st}: efyyyyyyyyyyyfyfyyyyyy and 2^{nd}: eeyyyyyyyffyyyyyyyyee; 1^{st}: efyyyyyyyyyyyfyfyyyyye and 2^{nd}: eeyyyyfyfyfyyyyyyyyee; 1^{st}: efyyyyyyyyyyyfyfyyyyyy and 2^{nd}: yyyyyyfyfyyyyyyyyy; 1^{st}: efyyyyyyyyyyyfyfyyyyye and 2^{nd}: YyyyyyfyfyfyYYyyyyyyy; 1^{st}: efyyyyyyyyyyyfyfyyyyyy and 2^{nd}: eeyyyyfyfyfyyyyyyyCC; 1^{st}: efyydydyyyyyyfyfyyyyye and 2^{nd}: eeyyyyfyfyfyyyyyyyyee; 1^{st}: efyydydyyyyyyfyfyyyyyy and 2^{nd}: yyyyyyfyfyfyyyyyyyyyy; 1^{st}: efyydydyyyyyyfyfyyyyye and 2^{nd}: yyyyyfyfyfyyyyyyyyyy; 1^{st} efyydydyyyyyyfyfyyyyyy and 2^{nd}: eeyyyyfyfyfyyyyyyyyee; 1^{st}: efyydydyyyyyyfyfyyyyye and 2^{nd}: eeyyyyyyyffyyyyyyyyee; 1^{st}: efyydydyyyyyyfyfyyyyyy and 2^{nd}: yyyyyyyyyffyyyyy yy; 1^{st}: efyydydyyyyyyfyfyyyyye and 2^{nd}: g yyyyyffyyyyyy; 1′: efyydydyyyyyyfyfyyyyyy and 2^{nd}: eeyyyyyyyffyyyyyyyyee; 1^{st}: efyydyyyyyyyfyfyyyyye and 2^{nd} eeyyyyffyyyyyyyyee; 1^{st}: efyyydyyyyyyyfyfyyyyyy and 2^{nd}: yyyyyyyyyffyyyyyyyyyy; 1^{st}: efyyydyyyyyyyfyfyyyyye and 2^{nd}: yyyyyyyyyffyyyyyyyyyy; 1^{st}: efyyydyyyyyyyfyfyyyyyy and 2^{nd}: eeyyyyyyyffyyyyee; 1^{st}: efyyydyyyyyyyfyfyyyyye and 2^{nd} eeyyyyfyfyfyyyyyyyyee; 1^{st}: efyyydyyyyyyyfyfyyyyyy and 2^{nd} YyyyyyfyfyfyYYyyyyyyy; 1^{st}: efyyydyyyyyyyfyfyyyyye and 2^{nd}. yyyyyfyfyfyyyyyyy; 1^{st}: efyyydyyyyyyyfyfyyyyyy and 2^{nd} eeyyyyfyfyfyyyyyyyyee; 1^{st}: efyyyyyyyyyyyfyfyyyyy and 2^{nd} eeyyyyyyyffyyyyyyyyee; 1^{st}: efyyyyyyyyyfyfyyyyy and 2^{nd}. yyyyyyyffyyyyyyyyyy; 1^{st}: efyyyyyyyyyyyfyfyyyyy and 2^{nd}: eeyyyyfyfyfyyyyyyee; 1^{st}: efyyyyyyyyyyyfyfyyyyy and 2^{nd} yyyyyyfyfyfyyyyyyyyyy; 1^{st}: efyydydyyyyyyfyfyyyyy and 2^{nd}. eeyyyyfyfyfyyyyyyyyee; 1^{st}. efyydydyyyyyyfyfyyyyy and 2^{nd} yyyyyyfyfyfyyyyyyyyyy; 1^{st}: efyydydyyyyyyfyfyyyyy and 2^{nd} eeyyyyyyyffyyyyyyyyee; 1^{st}: efyydydyyyyyyfyfyyyyy and 2^{nd} yyyyyyyyffyyyyyyyyyy; 1^{st} efyyydyyyyyyyfyfyyyyy and 2^{nd} eeyyyyyyyffyyyyyyyyee; 1^{st}: efyyydyyyyyyyfyfyyyyy and 2^{nd} yyyyyyyffyyyyyyyyyy; 1^{st}: efyyydyyyyyyyfyfyyyyy and 2^{nd}. eeyyyyfyfyfyyyyyyyyee; 1^{st}: efyyydyyyyyyyfyfyyyyy and 2^{nd}: yyyyyyfyfyfyyyyyyyyyy; wherein each “y” represents a 2′-OMe sugar moiety, each “e” represents a 2′-MOE sugar moiety, each “f” represents a 2′-fluoro sugar moiety, and each “d” represents a 2′-deoxy sugar moiety.

Embodiment 183. The oligomeric duplex of any one of embodiments 1-4, 12, 18, 19, 30-69, 72, 80, 105-115, 117-139, 152, 153, and 175-181, wherein the first modified oligonucleotide has a first sugar motif (1^{st}) and the second modified oligonucleotide has a second sugar motif (2^{nd}) and the first and second sugar motifs are selected from among the following combinations (5′ to 3′): 1^{st}: efyydydyyyyyyfyfyyyyyee and 2^{nd}: eeyyyyfyfffyyyyyyyyee; 1^{st}: efyydydyyyyyyfyfyyyyyyy and 2^{nd}: yyyyyyfyfffyyyyyyyyyy; 1^{st} efyydydyyyyyyfyfyyyyyee and 2^{nd}: yyyyyfyfffyyyyyyyyyy; 1^{st}: efyydydyyyyyyfyfyyyyyyy and 2^{nd}: eeyyyyfyfffyyyyyyyyee; 1^{st}: efyyyyyyyyyyyfyfyyyyyee and 2^{nd}: eeyyyyfyfffyyyyyyyyee; 1^{st}: efyyyyyyyyyyyfyfyyyyyyy and 2^{nd}: yyyyyyfyfffyyyyyyyyyy; 1^{st}: efyyyyyyyyyyyfyfyyyyyee and 2^{nd}: yyyyyyfyfffyyyyyyyyyy; 1^{st}: efyyyyyyyyyyyfyfyyyyyyy and 2^{nd}. eeyyyyfyfffyyyyyyyyee; 1^{st}: efyyyyyyyyyyyfyfyyyyyee and 2^{nd}: eeyyyyyyyffyyyyyyyyee; 1^{st}: efyyyyyyyyyyyfyfyyyyyyy and 2^{nd}: yyyyyyyyyffyyyyyyyyyy; 1^{st}: efyyyyyyyyyyyfyfyyyyyee and 2^{nd}: yyyyyyyyyffyyyyyyyyyy; 1^{st}: efyyyyyyyyyyyfyfyyyyyyy and 2^{nd}: eeyyyyyyyffyyyyyyyyee; 1^{st}: efyyyyyyyyyyyfyfyyyyyee and 2^{nd}: eeyyyyfyfyfyyyyyyyee; 1^{st}: efyyyyyyyyyyyyfyyyyyy and 2^{nd}: yyyyyyfyfyfyyyyyyyyyy; 1^{st}: efyyyyyyyyyyyfyfyyyyyee and 2^{nd}: yyyyyyfyfyfyyyyyyyyyy; 1^{st}: efyyyyyyyyyyyfyfyyyyyyy and 2^{nd}: eeyyyyfyfyfyyyyyyyyee; 1^{st}: efyyyfyyyyyyyfyfyyyyyyy and 2^{nd}: yyyyyyyyyffyyyyyyyyyy; 1^{st}: efyyyfyyyyyyyfyfyyyyyee and 2^{nd}. eeyyyyyyyffyyyyyyyyee; 1^{st}: efyyyfyyyyyyyfyfyyyyyyy and 2^{nd}. eeyyyyyyyffyyyyyyyyee; 1^{st}: efyyyfyyyyyyyfyfyyyyyee and 2^{nd}: yyyyyyyyyffyyyyyyyyyy; 1^{st}: efyyyfyyyyyyyfyfyyyyyyy and 2^{nd}: yyyyyyfyfyfyyyyyyyyyy; 1^{st}: efyyyfyyyyyyyfyfyyyyyee and 2^{nd}: eeyyyyfyfyfyyyyyyyyee; 1^{st}: efyyyfyyyyyyyfyfyyyyyyy and 2^{nd}: eeyyyyfyfyfyyyyyyyyee; 1^{st}: efyyyfyyyyyyyfyfyyyyyee and 2^{nd}: yyyyyfyfyfyyyyyyyyyy; 1^{st}: efyyyfyyyyyyyfyfyyyee and 2^{nd}: eeyyyyfyfffyyyyyyee; 1^{st}: efyyyfyyyyyyyfyfyyyyy and 2^{nd}: yyyyyyfyfffyyyyyyyy; 1^{st}: efyyyfyyyyyyyfyfyyyee and 2^{nd}: yyyyyyfyfffyyyyyyyy; 1^{st}: efyyyfyyyyyyyfyfyyyyy and 2^{nd}: eeyyyyfyfffyyyyyyee; 1^{st}: efyyyfyyyyyyyfyfyyyee and 2^{nd}: eeyyyyffyyyyyyyyee; 1^{st}: efyyyfyyyyyyyfyfyyyyy and 2^{nd}: yyyyyyffyyyyyyyyyy; 1^{st}: efyyyfyyyyyyyfyfyyyee and 2^{nd}: yyyyyyffyyyyyyyyyy; 1^{st}: efyyyfyyyyyyyfyfyyyyy and 2^{nd}: eeyyyyffyyyyyyyyee; 1^{st}: e[FHNA]yydydyyyyfyfyfyyyyyee and 2^{nd}: eeyyyyfyfffyyyyyyyyee; 1^{st}: efyyyfyyyyyyyfyfyyyyyey and 2^{nd}: eeyyyyfyfffyyyyyyyyee; 1^{st}: efyyyfyyyyyyfyfyyyyyey and 2^{nd}: yyyyyyfffyyyyyyyyyy; 1^{st}: e[FHNA]yydydyyyyfyfyfyyyyyyy and 2^{nd} yyyyyyfyfffyyyyyyyyyy; 1^{st}: e[FHNA]yydydyyyyfyfyfyyyyyee and 2^{nd}: yyyyyyfyfffyyyyyyyyyy; 1^{st}: e[FHNA]yydydyyyyfyfyfyyyyyyy and 2^{nd} eeyyyyfyfffyyyyyyyyee; 1^{st}: e[FHNA]yydydyyyyfyfyfyyyyyee and 2^{nd} eeyyyyfyfyfyyyyyyyyee; 1^{st}: e[FHNA]yydydyyyyfyfyfyyyyyyy and 2^{nd} yYYYYYfYfYfYYYy yYYYY; 1^{st}: e[FHNA]yydydyyyyfyfyfyyyyyee and 2^{nd} yyyyyyfyfyfyyyyyyyyy; and 1^{st}: e[FHNA]yydydyyyyfyfyfyyyyyyy and 2^{nd}: eeyyyyfyfyfyyyyyyyyee, wherein each “y” represents a 2′-OMe sugar moiety, each “e” represents a 2′-MOE sugar moiety, each “f” represents a 2′-fluoro sugar moiety, each “[FHNA]” represents a 3′-fluoro-hexitol sugar moiety, and each “d” represents a 2′-deoxy sugar moiety.

Embodiment 184. The oligomeric duplex of any one of embodiments 1-4, 12, 18, 19, 30-69, 72, 80, 105-115, 117-139, 152, 153, and 175-181, wherein the first modified oligonucleotide has a first sugar motif (1^{st}) and the second modified oligonucleotide has a second sugar motif (2^{nd}) and the first and second sugar motifs are selected from among the following combinations (5′ to 3′): 1^{st}: efyyyfyyyyyyyfyfyyyyyyy and 2^{nd}: yyyyyyfffyyyyyyyyyy; 1^{st}: efyyyfyyyyyyyfyfyyyyyee and 2^{nd}: eeyyyyfyfffyyyyyyyyee; 1^{st}: efyyyfyyyyyyyfyfyyyyyee and 2^{nd}: yyyyyyfyfffyyyyyyyyyy; and 1^{st}: efyyyfyyyyyyyfyfyyyyyyy and 2^{nd}: eeyyyyfyfffyyyyyyyyee, wherein each “y” represents a 2′-OMe sugar moiety, each “e” represents a 2′-MOE sugar moiety, and each “f” represents a 2′-fluoro sugar moiety.

Embodiment 185. The oligomeric duplex of any one of embodiments 1-4, 12, 18, 19, 30-69, 72, 80, 105-115, 117-139, 152, 153, and 175-181, wherein the first modified oligonucleotide has a first sugar motif (Ist) and the second modified oligonucleotide has a second sugar motif (2^{nd}) and the first and second sugar motifs are selected from among the following combinations (5′ to 3′): 1^{st}: efyyyfyyyyyyyfyfyyyyyyy and 2^{nd}: yyyyyyyyyyyyyyy; 1^{st}: efyyyfyyyyyyyfyfyyyyyee and 2^{nd}: eeyyy yyyyyyyyyce; 1^{st}: efyyyfyyyyyyyfyfyyyyyyy and 2^{nd}: eeyyyyyyyyyyyyyyyyyee; 1′t: efyyyfyyyyyyyfyfyyyyyee and 2^{nd}: yyyyyyyyyyyyyyyyyyyyy; 1^{st}: efyyyfyyyyyyyfyfyyyyyyy and 2^{nd}: yy yyyyyydyyyyyyyyyy; 1^{st}: efyyyfyyyyyyyfyfyyyyyee and 2^{nd}: eeyyyyyyyydyyyyyyyyee; 1^{st}: efyyyfyyyyyyyfyfyyyyyyy and 2^{nd}: eeyyyyyyyydyyyyyyyyee; 1^{st}: efyyyfyyyyyyyfyfyyyyyee and 2^{nd}: yyyyyyyyyydyyyyyyyyyy; 1^{st}: efyyyyyyyyyyyfyyyyyyyee and 2^{nd}: eeyyyyfyfffyyyyyyyyee; 1^{st}: efyyyyyyyyyyyfyyyyyyyyy and 2^{nd}: yyyyyyfyfffyyyyyyyyyy; 1^{st}: efyyyyyyyyyyyfyyyyyyyee and 2^{nd}: yyyyyfyfffyyyyyyyyyy; 1^{st}: efyyyyyyyyyyyfyyyyyyyyy and 2^{nd}: eeyyyyfyfffyyyyyyyyee; 1^{st}: efyyyyyyyyyyyfyyyyyyyee and 2^{nd}: eeyyyyfyfyfyyyyyyyyee; 1^{st}: efyyyyyyyYyyyfyyyyyyyyy and 2^{nd}: yyyyyyfyfyfyyyyyyyyyy; 1^{st}: efyyyyyyyyyyyfyyyyyyyee and 2^{nd}: yyyyyyfyfyfyyyyyyyy. 1^{st}: efyyyyyyyyyyyfyyyyyyyyy and 2^{nd}: eeyyyyfyfyfyyyyyyyyee; 1^{st}: efyyydyyyyyyfyfyyyyyee and 2^{nd}: eeyyyyfyfffyyyyyyyyee; 1^{st}: efyyydyyyyyyyfyfyyyyyyy and 2^{nd}: yyyyyyfyfffyyyyyyyyyy; 1^{st}: efyyydyyyyyyyfyfyyyyyee and 2^{nd}: yyyyyyfyfffyyyyyyyyyy; 1^{st}: efyyydyyyyyyyfyfyyyyyyy and 2^{nd}: eeyyyyfyfffyyyyyyyyee; 1^{st}: efyyyfyyyyyyyfyfyyyee and 2^{nd}: eeyyfyfffyyyyyyyyee; 1^{st}: efyyyfyyyyyyyfyfyyyyy and 2^{nd}: yyyyfyfffyyyyyyyyyy; 1^{st}: efyyyfyyyyyyyfyfyyyee and 2^{nd}: yyyyfyfffyyyyyyyyyy; 1^{st}: efyyyfyyyyyyyfyfyyyyy and 2^{nd}: eeyyfyfffyyyyyyyyee; 1^{st}: efyyyfyyyyyyyfydyyyyyee and 2^{nd}: eeyyyyfyfffyyyyyyyyee; 1^{st}: efyyyfyyyyyfydyyyyyyy and 2^{nd}: yyyfyfffyyyyyyyyyy 1^{st}: efyyyfyyyyyyyfydyyyyyee and 2ⁿd yyyyyyfyfffyyyyyyyyyy; 1^{st}: efyyyfyyyyyyyfydyyyyyyy and 2ⁿd eeyyyyfyfffyyyyyyyyee; 1^{st}: e[FHNA]yyyfyyyyyyyfyyyyyee and 2^{nd}: eeyyyyfyfffyyyyyyyyee; 1^{st}: e[FHNA]yyyfyyyyyyyfyfyyyyyyy and 2^{nd}: yyyyyyfyfffyyyyyyyyyy; 1^{st}: e[FHNA]yyyfyyyyyyyfyfyyyyyee and 2^{nd}: yyyyyyfyfffyyyyyyyyyy; and 1^{st}: e[FHNA]yyyfyyyyyyyfyfyyyyyyy and 2^{nd}: eeyyyyfyfffyyyyyyyyee, wherein each “y” represents a 2′-OMe sugar moiety, each “e” represents a 2′-MOE sugar moiety, each “f” represents a 2′-fluoro sugar moiety, each “[FHNA]” represents a 3′-fluoro-hexitol sugar moiety, and each “d” represents a 2′-deoxy sugar moiety.

Embodiment 186. The oligomeric duplex of any one of embodiments 1-4, 12, 18, 19, 30-69, 72, 80, 105-115, 117-139, 152, 153, and 175-181, wherein the first modified oligonucleotide has a first sugar motif (1^{st}) and the second modified oligonucleotide has a second sugar motif (2^{nd}) and the first and second sugar motifs are selected from among the following combinations (5′ to 3′): 1^{st}: efyyyyyyyyyyyfyyyyyyyee and 2^{nd}: eeyyyyyyyffyyyyyee; 1^{st}: efyyyyyyyyyyyfyyyyyyyyy and 2^{nd}: yy yyyffyyyyyyyyy. 1^{st}: efyyyyyyyyyyyfyyyyyyyee and 2^{nd}: yyyyyyyyffyyyyyyyyyy; 1^{st}: efyyyyyyyyyyyfyyyyyyyyy and 2^{nd}: eeyyyyyyyffyyyyyyyyee; 1^{st}: efyyyfyyyyyyyfyyyyyyyee and 2^{nd}: eeyyyyfyfffyyyyyyyyee; 1^{st}: efyyyfyyyyyyyfyyyyyyyyy and 2^{nd}: yyyyyyfyfffyyyyyyyyyy; 1^{st}: efyyyfyyyyyyyfyyyyyyyee and 2^{nd}: yyyyyyfyfffyyyyyyyyyy; 1^{st}: efyyyfyyyyyyyfyyyyyyyyy and 2^{nd}: eeyyyyffffyyyyyyyyee; 1^{st}: efyyyfyyyyyyyfyyyyyyyee and 2^{nd}: eeyyyyyyyffyyyyyyyyee; 1^{st}: efyyyfyyyyyyyfyyyyyyyyy and 2^{nd}: yyyyyyyyyffyyyyyyyyyy; 1^{st}: efyyyfyyyyyyyfyyyyyyyee and 2^{nd}: yyyyyyfyfffyyyyyyyyyy; and 1^{st}: efyyyfyyyyyyyfyyyyyyyyy and 2^{nd}: eeyyyyfyfffyyyyyyyyee, wherein each “y” represents a 2′-OMe sugar moiety, each “e” represents a 2′-MOE sugar moiety, and each “f” represents a 2′-fluoro sugar moiety.

Embodiment 187. The oligomeric duplex of any one of embodiments 123-186, wherein the internucleoside linkages between the first and second nucleosides and the second and third nucleosides counting from the 5′ end of the first modified oligonucleotide, and the internucleoside linkages between the first and second nucleosides and the second and third nucleosides counting from the 3′ end of the first modified oligonucleotide are phosphorothioate internucleoside linkages and wherein all other internucleoside linkages of the first modified oligonucleotide are phosphodiester internucleoside linkages.

Embodiment 188. The oligomeric duplex of any one of embodiments 123-187, wherein the internucleoside linkages between the first and second nucleosides and the second and third nucleosides counting from the 5′ end of the second modified oligonucleotide, and the internucleoside linkages between the first and second nucleosides and the second and third nucleosides counting from the 3′ end of the second modified oligonucleotide are phosphorothioate internucleoside linkages and wherein all other internucleoside linkages of the second modified oligonucleotide are phosphodiester internucleoside linkages.

Embodiment 189. The oligomeric duplex of any one of embodiments 123-188, wherein the nucleobase sequence of the first modified oligonucleotide comprises or consists of the nucleobase sequence of any one of SEQ ID NOs: 967, 968, 971, 972, 974, 975, 1033-1038, 1254-1255, or 1258-1287.

Embodiment 190. The oligomeric duplex of any one of embodiments 1-189, wherein a cytosine nucleobase in the first and/or second modified oligonucleotide is optionally 5-methylcytosine.

Embodiment 191. The oligomeric duplex of any one of embodiments 123-190, wherein the oligomeric duplex comprises a conjugate group comprising a conjugate moiety and a conjugate linker.

Embodiment 192. The oligomeric duplex of embodiment 191, wherein the conjugate moiety comprises an active drug substance, an aliphatic chain, a lipid, a peptide, a protein, a hydrocarbon, a polyamine, a polyamide, a polyether, a thioether, an aptamer, an antibody, an antibody fragment, a vitamin, a fatty acid, a carbohydrate, an intercalator or a reporter molecule.

Embodiment 193. The oligomeric duplex of embodiment 191, wherein the conjugate group comprises a C22 alkyl, C20 alkyl, C17 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, or C5 alkyl, wherein the alkyl chain optionally has one or more unsaturated bonds.

Embodiment 194. The oligomeric duplex of embodiment 191, wherein the conjugate group comprises a 6-palmitamidohexyl moiety or a 2-(hydroxymethyl)-6-palmitamidohexyl moiety.

Embodiment 195. The oligomeric duplex of embodiment 191, wherein the oligomeric duplex comprises a conjugate moiety that binds type 1 transferrin receptor (TfR1).

Embodiment 196. The oligomeric duplex of embodiment 195, wherein the conjugate moiety is selected from an antibody or fragment thereof, a protein or peptide, and an aptamer capable of binding TfR1.

Embodiment 197. The oligomeric duplex of embodiment 196, wherein the conjugate moiety is a cyclic protein or cyclic peptide.

Embodiment 198. The oligomeric duplex of embodiment 195, wherein the conjugate group comprises a bicycle ligand and a conjugate linker.

Embodiment 199. The oligomeric duplex of embodiment 198, wherein the bicycle ligand comprises a peptide consisting of 13-22 linked amino acids or amino acid mimetics and a molecular scaffold, wherein each of a first, a second, and a third amino acid of the peptide comprises a reactive group, each of which separately forms a bond with the molecular scaffold, thereby forming two peptide loops attached to the molecular scaffold.

Embodiment 200. The oligomeric duplex of embodiment 199 wherein the bicycle ligand comprises an amino acid sequence selected from any one of SEQ ID NO: 1062-1253.

Embodiment 201. The oligomeric duplex of any one of embodiments 198-200, wherein the conjugate linker comprises a (bicyclo[6.1.0]nonyne)-formyl (BCN) moiety.

Embodiment 202. The oligomeric duplex of any one of embodiments 198-200, wherein the conjugate linker comprises a 6-(BCN-carbamate)-2-(hydroxymethyl)hexyl phosphoryl moiety or a 6-(BCN-carbamate)hexyl phosphoryl moiety.

Embodiment 203. The oligomeric duplex of any one of embodiments 123-190, comprising a conjugate group consisting of a conjugate moiety and a conjugate linker wherein the conjugate linker comprises a 6-(BCN-carbamate)-2-(hydroxymethyl)hexyl phosphoryl moiety or a 6-(BCN-carbamate)hexyl phosphoryl moiety.

Embodiment 204. The oligomeric duplex of any one of embodiments 123-190, comprising a bicycle ligand comprising a peptide having an amino acid sequence selected from any one of SEQ ID NOs: 1062-1253 and wherein each cysteine forms a covalent bond with the molecular scaffold 1,1′,1″-(1,3,5-triazinane-1,3,5-triyl)tris(2-bromoethanone) (TATB) or the molecular scaffold is 1,1′,1″-(1,3,5-triazinane-1,3,5-triyl)triprop-2-en-1-one (TATA).

Embodiment 205. An oligomeric compound comprising a modified oligonucleotide according to the following chemical notation:

(SEQ ID NO: 1033)
vPTₑₛU_{fs}U_{yo}A_{yo}A_{yo}G_{do}A_{yo}U_{yo}G_{yo}A_{yo}G_{yo}A_{yo}C_{yo}A_{fo}G_{yo}A_{fo}
A_{yo}A_{yo}U_{yo}U_{yo}G_{ys}AₑsA^{e},

wherein:
A=an adenine nucleobase,
C=a cytosine nucleobase,
G=a guanine nucleobase,
T=a thymine nucleobase,
U=a uracil nucleobase,
d=a 2′-deoxy sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate group.

Embodiment 206. An oligomeric compound comprising a modified oligonucleotide according to the following chemical notation: mCesAₑₛA_{yo}U_{yo}U_{yo}U_{yo}C_{yo}U_{yo}G_{yo}U_{fo}C_{fo}U_{yo}C_{yo}A_{yo}U_{yo}C_{yo}U_{yo}U_{yo}A_{ys}AₑₛAₑ (SEQ ID NO: 1027), wherein:
A=an adenine nucleobase,
^{m}C=a 5-methyl cytosine nucleobase,
C=a cytosine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

Embodiment 207. An oligomeric compound comprising a conjugated modified oligonucleotide according to the following chemical notation: [X]ₙ-^{m}CₑₛAₑₛA_{yo}U_{yo}U_{yo}U_{yo}C_{yo}U_{yo}G_{yo}U_{fo}C_{fo}U_{yo}C_{yo}A_{yo}U_{yo}C_{yo}U_{yo}U_{yo}A_{ys}AₑₛAₑ-[X]ₖ (SEQ ID NO: 1039), wherein:
^{m}C=a 5-methyl cytosine nucleobase,
C=a cytosine nucleobase,
A=an adenine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage,
[X]=a conjugate group comprising a bicycle ligand, and
n and k are 0 or 1, wherein if n=1 then k=0, and if n=0 then k=1.

Embodiment 208. An oligomeric duplex comprising the oligomeric compound according to embodiment 205 and the oligomeric compound according to embodiment 206.

Embodiment 209. An oligomeric duplex comprising the oligomeric compound according to embodiment 205 and the oligomeric compound according to embodiment 207.

Embodiment 210. An oligomeric compound comprising a modified oligonucleotide according to the following chemical notation: vP-TₑₛU_{fs}U_{yo}A_{yo}A_{do}G_{yo}A_{do}U_{yo}G_{yo}A_{yo}G_{yo}A_{yo}C_{yo}A_{fo}G_{yo}A_{fo}A_{yo}A_{yo}U_{yo}U_{yo}G_{ys}AₑₛAₑ (SEQ ID NO: 1034), wherein:
A=an adenine nucleobase,
C=a cytosine nucleobase,
G=a guanine nucleobase,
T=a thymine nucleobase,
U=a uracil nucleobase,
d=a 2′-deoxy sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate group.

Embodiment 211. An oligomeric compound comprising a modified oligonucleotide according to the following chemical notation: CₑₛAₑₛA_{yo}U_{yo}U_{yo}U_{yo}C_{fo}U_{yo}G_{fo}U_{yo}C_{fo}U_{yo}C_{yo}A_{yo}U_{yo}C_{yo}U_{yo}U_{yo}A_{ys}AₑₛAₑ (SEQ ID NO: 1028), wherein:
A=an adenine nucleobase,
C=a cytosine nucleobase,
G=a guanine nucleobase,
U=a uracil nucleobase,
e=a 2′-MOE sugar moiety,
f=a 2′-fluoro sugar moiety,
y=a 2′-Ome sugar moiety,
o=a phosphodiester internucleoside linkage, and
s=a phosphorothioate internucleoside linkage.

Embodiment 212. An oligomeric compound comprising a conjugated modified oligonucleotide according to the following chemical notation: [X],-CₑₛAₑₛA_{yo}U_{yo}U_{yo}U_{yo}C_{fo}U_{yo}G_{fo}U_{yo}C_{fo}U_{yo}C_{yo}A_{yo}U_{yo}C_{yo}U_{yo}U_{yo}A_{ys}AₑₛAₑ-[X]ₖ (SEQ ID NO: 1040), wherein:
C=a cytosine nucleobase,
A=an adenine nucleobase,
G=a guanine nucleobase,
U=a uracil nucleobase,
e=a 2′-MOE sugar moiety,
f=a 2′-fluoro sugar moiety,
y=a 2′-Ome sugar moiety,
o=a phosphodiester internucleoside linkage,
s=a phosphorothioate internucleoside linkage,
[X]=a conjugate group comprising a bicycle ligand, and
n and k are 0 or 1, wherein if n=1 then k=0, and if n=0 then k=1.

Embodiment 213. An oligomeric duplex comprising an oligomeric compound of embodiment 210 and an oligomeric compound of embodiment 211.

Embodiment 214. An oligomeric duplex comprising an oligomeric compound of embodiment 210 and an oligomeric compound of embodiment 212.

Embodiment 215. An oligomeric compound comprising a modified oligonucleotide according to the following chemical notation: vPTₑₛA_{fs}U_{yo}A_{yo}A_{yo}A_{do}U_{yo}A_{yo}G_{yo}A_{yo}U_{yo}U_{yo}C_{yo}U_{fo}G_{yo}U_{fo}A_{yo}G_{yo}C_{yo}U_{yo}U_{ys}AₑₛAₑ (SEQ ID NO: 1035), wherein:
A=an adenine nucleobase,
C=a cytosine nucleobase,
G=a guanine nucleobase,
T=a thymine nucleobase,
U=a uracil nucleobase,
d=a 2′-deoxy sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate group.

Embodiment 216. An oligomeric compound comprising a modified oligonucleotide according to the following chemical notation: AₑₛAₑₛG_{yo}C_{yo}U_{yo}A_{yo}C_{yo}A_{yo}G_{yo}A_{fo}A_{fo}U_{yo}C_{yo}U_{yo}A_{yo}U_{yo}U_{yo}U_{yo}A_{ys}TₑₛAₑ (SEQ ID NO: 1029), wherein:
A=an adenine nucleobase,
C=a cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

Embodiment 217. An oligomeric compound comprising a conjugated modified oligonucleotide according to the following chemical notation: [X]ₙ-AₑₛAₑₛG_{yo}C_{yo}U_{yo}A_{yo}C_{yo}A_{yo}G_{yo}A_{fo}A_{fo}U_{yo}C_{yo}U_{yo}A_{yo}U_{yo}U_{yo}U_{yo}A_{ys}TₑₛAₑ-[X]ₖ (SEQ ID NO: 1041),

wherein:
C=a cytosine nucleobase,
A=an adenine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage,
[X]=a conjugate group comprising a bicycle ligand, and
n and k are 0 or 1, wherein if n=1 then k=0, and if n=0 then k=1.

Embodiment 218. An oligomeric duplex comprising an oligomeric compound of embodiment 215 and an oligomeric compound of embodiment 216.

Embodiment 219. An oligomeric compound comprising an oligomeric compound of embodiment 215 and an oligomeric compound of embodiment 217.

Embodiment 220. An oligomeric compound comprising a modified oligonucleotide according to the following chemical notation: vPTₑₛA_{fs}U_{yo}A_{yo}A_{do}A_{yo}T_{do}A_{yo}G_{yo}A_{yo}U_{yo}U_{yo}C_{yo}U_{fo}G_{yo}U_{fo}A_{yo}G_{yo}C_{yo}U_{yo}U_{ys}AₑₛAₑ (SEQ ID NO: 1036), wherein:
A=an adenine nucleobase,
C=a cytosine nucleobase,
G=a guanine nucleobase,
T=a thymine nucleobase,
U=a uracil nucleobase,
d=a 2′-deoxy sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate group.

Embodiment 221. An oligomeric compound comprising a modified oligonucleotide according to the following chemical notation: AₑₛAₑₛG_{yo}C_{yo}U_{yo}A_{yo}C_{fo}A_{yo}G_{fo}A_{yo}A_{fo}U_{yo}C_{yo}U_{yo}A_{yo}U_{yo}U_{yo}U_{yo}A_{ys}UₑₛAₑ (SEQ ID NO: 1030), wherein:
A=an adenine nucleobase,
C=a cytosine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

Embodiment 222. An oligomeric compound comprising a conjugated modified oligonucleotide according to the following chemical notation: [X]ₙ-AₑₛAₑₛG_{yo}C_{yo}U_{yo}A_{yo}C_{fo}A_{yo}G_{fo}A_{yo}A_{fo}U_{yo}C_{yo}U_{yo}A_{yo}U_{yo}U_{yo}U_{yo}A_{ys}UₑₛAₑ-[X]ₖ (SEQ ID NO: 1042), wherein:
C=a cytosine nucleobase,
A=an adenine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage,
[X]=a conjugate group comprising a bicycle ligand, and
n and k are 0 or 1, wherein if n=1 then k=0, and if n=0 then k=1.

Embodiment 223. An oligomeric duplex comprising an oligomeric compound of embodiment 220 and an oligomeric compound of embodiment 221.

Embodiment 224. An oligomeric duplex comprising an oligomeric compound of embodiment 220 and an oligomeric compound of embodiment 222.

Embodiment 225. An oligomeric compound comprising a modified oligonucleotide according to the following chemical notation: vPTₑₛU_{fs}A_{yo}A_{yo}G_{yo}T_{do}U_{yo}U_{yo}U_{yo}A_{yo}G_{yo}U_{yo}C_{yo}U_{fo}U_{yo}A_{fo}A_{yo}U_{yo}C_{yo}U_{yo}U_{ys}AₑₛAₑ (SEQ ID NO: 1037), wherein:
A=an adenine nucleobase,
C=a cytosine nucleobase,
G=a guanine nucleobase,
T=a thymine nucleobase,
U=a uracil nucleobase,
d=a 2′-deoxy sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate group.

Embodiment 226. An oligomeric compound comprising a modified oligonucleotide according to the following chemical notation: AₑₛAₑₛG_{yo}A_{yo}U_{yo}U_{yo}A_{yo}A_{yo}G_{yo}A_{fo}C_{fo}U_{yo}A_{yo}A_{yo}A_{yo}A_{yo}C_{yo}U_{yo}U_{ys}AₑₛAₑ (SEQ ID NO: 1031), wherein:
A=an adenine nucleobase,
C=a cytosine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

Embodiment 227. An oligomeric compound comprising a conjugated modified oligonucleotide according to the following chemical notation: [X]ₙ-AₑₛAₑₛG_{yo}A_{yo}U_{yo}U_{yo}A_{yo}A_{yo}G_{yo}A_{fo}C_{fo}U_{yo}A_{yo}A_{yo}A_{yo}A_{yo}C_{yo}U_{yo}U_{ys}AₑₛAₑ-[X]ₖ (SEQ ID NO: 1043), wherein:
C=a cytosine nucleobase,
A=an adenine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage,
[X]=a conjugate group comprising a bicycle ligand, and
n and k are 0 or 1, wherein if n=1 then k=0, and if n=0 then k=1.

Embodiment 228. An oligomeric duplex comprising the modified oligonucleotide according to embodiment 225 and the modified oligonucleotide according to embodiment 226.

Embodiment 229. An oligomeric duplex comprising the modified oligonucleotide according to embodiment 225 and the conjugate according to embodiment 227.

Embodiment 230. An oligomeric compound comprising a modified oligonucleotide according to the following chemical notation: vPTₑₛU_{fs}A_{yo}A_{yo}G_{do}U_{yo}T_{do}U_{yo}U_{yo}A_{yo}G_{yo}U_{yo}C_{yo}U_{fo}U_{yo}A_{fo}A_{yo}U_{yo}C_{yo}U_{yo}U_{ys}AₑₛAₑ (SEQ ID NO: 1038), wherein:
A=an adenine nucleobase,
C=a cytosine nucleobase,
G=a guanine nucleobase,
T=a thymine nucleobase,
U=a uracil nucleobase,
d=a 2′-deoxy sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate group.

Embodiment 231. An oligomeric compound comprising a modified oligonucleotide according to the following chemical notation: AₑₛAₑₛG_{yo}A_{yo}U_{yo}U_{yo}A_{fo}A_{yo}G_{fo}A_{yo}C_{fo}U_{yo}A_{yo}A_{yo}A_{yo}A_{yo}C_{yo}U_{yo}U_{ys}AₑₛAₑ (SEQ ID NO: 1032), wherein:
A=an adenine nucleobase,
C=a cytosine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

Embodiment 232. An oligomeric compound comprising a conjugated modified oligonucleotide according to the following chemical notation: [X]ₙ-AₑₛAₑₛG_{yo}A_{yo}U_{yo}U_{yo}A_{fo}A_{yo}G_{fo}A_{yo}C_{fo}U_{yo}A_{yo}A_{yo}A_{yo}A_{yo}C_{yo}U_{yo}U_{ys}AₑₛAₑ-[X]ₖ (SEQ ID NO: 1044), wherein:
C=a cytosine nucleobase,
A=an adenine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage,
[X]=a conjugate group comprising a bicycle ligand, and
n and k are 0 or 1, wherein if n=1 then k=0, and if n=0 then k=1.

Embodiment 233. An oligomeric duplex comprising an oligomeric compound of embodiment 230 and an oligomeric compound of embodiment 231.

Embodiment 234. An oligomeric duplex comprising an oligomeric compound of embodiment 230 and an oligomeric compound of embodiment 232.

Embodiment 235. An oligomeric compound comprising a modified oligonucleotide according to the following chemical notation: vPTₑₛA_{fs}U_{yo}A_{yo}A_{yo}A_{yo}U_{yo}A_{yo}G_{yo}A_{yo}U_{yo}U_{yo}C_{yo}U_{fo}G_{yo}U_{fo}A_{yo}G_{yo}C_{yo}U_{yo}U_{ys}AₑₛAₑ (SEQ ID NO: 1255), wherein:
A=an adenine nucleobase,
C=a cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate group.

Embodiment 236. An oligomeric compound comprising a modified oligonucleotide according to the following chemical notation: AₑₛAₑₛG_{yo}C_{yo}U_{yo}A_{yo}C_{yo}A_{yo}G_{yo}A_{fo}A_{fo}U_{yo}C_{yo}U_{yo}A_{yo}U_{yo}U_{yo}U_{yo}A_{ys}TₑₛAₑ (SEQ ID NO: 1029), wherein:
A=an adenine nucleobase,
C=a cytosine nucleobase,
G=a guanine nucleobase,
U=a uracil nucleobase,
T=a thymine nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

Embodiment 237. An oligomeric compound comprising a conjugated modified oligonucleotide according to the following chemical notation: [X]ₙ-AₑₛAₑₛG_{yo}C_{yo}U_{yo}A_{yo}C_{yo}A_{yo}G_{yo}A_{fo}A_{fo}U_{yo}C_{yo}U_{yo}A_{yo}U_{yo}U_{yo}U_{yo}A_{ys}TₑₛAₑ-[X]ₖ (SEQ ID NO: 1041), wherein:
C=a cytosine nucleobase,
A=an adenine nucleobase,
G=a guanine nucleobase,
U=a uracil nucleobase,
T=a thymine nucleobase,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage,
[X]=a conjugate group comprising a bicycle ligand, and
n and k are 0 or 1, wherein if n=1 then k=0, and if n=0 then k=1.

Embodiment 238. An oligomeric duplex comprising the oligomeric compound according to embodiment 235 and the oligomeric compound according to embodiment 236.

Embodiment 239. An oligomeric duplex comprising the oligomeric compound according to embodiment 235 and the oligomeric compound according to embodiment 237.

Embodiment 240. The oligomeric duplex of any one of embodiments 123-190, 209, 214, 219, 224, 229, 234, 239, or the oligomeric compound of any one of embodiments 207, 212, 217, 222, 227, 232, or 237, comprising a bicycle ligand or conjugate group having the following structure:

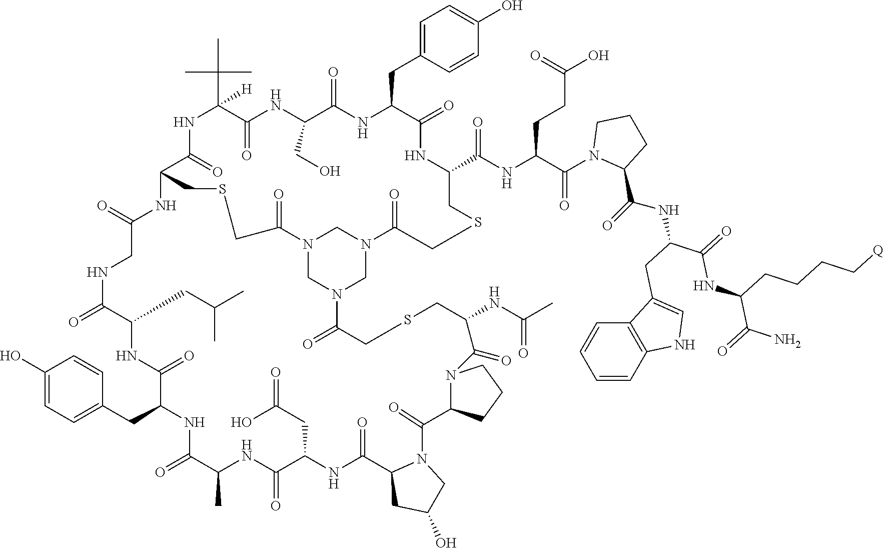

or a salt thereof, wherein Q is N₃ (BCY17901, SEQ ID NO: 1045), NH₂ (BCY21757, SEQ ID NO: 1202), SH (BCY21758, SEQ ID NO:1203), or a conjugate linker, or a conjugate linker covalently connected to an oligonucleotide.

Embodiment 241. The oligomeric duplex or oligomeric compound of embodiment 240, wherein Q is N₃ or a conjugate linker.

Embodiment 242. The oligomeric duplex of any one of embodiments 123-190, 209, 214, 219, 224, 229, 234, 239, or the oligomeric compound of any one of embodiments 207, 212, 217, 222, 227, 232, or 237, comprising a conjugate group having the following structure:

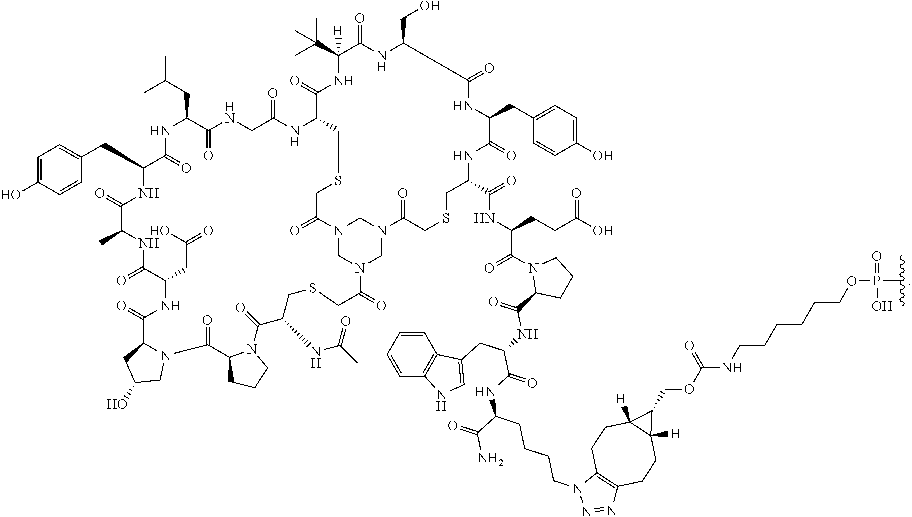

Embodiment 243. The oligomeric duplex of any one of embodiments 123-190, 209, 214, 219, 224, 229, 234, 239, or the oligomeric compound of any one of embodiments 207, 212, 217, 222, 227, 232, or 237, comprising a conjugate group having the following structure:

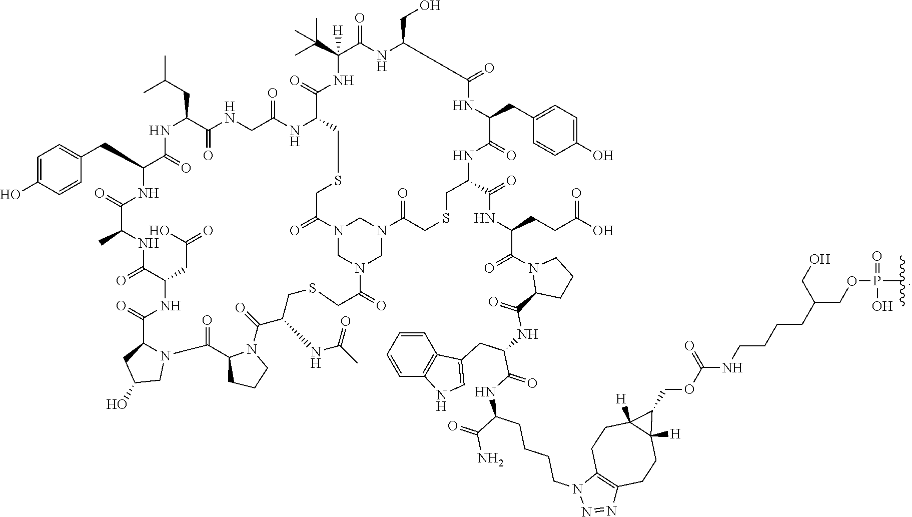

Embodiment 244. The oligomeric duplex or oligomeric compound of any one of embodiments 191-243, wherein the conjugate group is attached to the second modified oligonucleotide.

Embodiment 245. The oligomeric duplex or compound of any one of embodiments 191-243, wherein the conjugate group is attached to the 5′- or 3′-end of the second modified oligonucleotide.

Embodiment 246. The oligomeric duplex or oligomeric compound of any one of embodiments 191-243, wherein the conjugate group is attached to the 5′-terminal nucleoside of the second modified oligonucleotide.

Embodiment 247. An oligomeric duplex according to the following chemical structure:

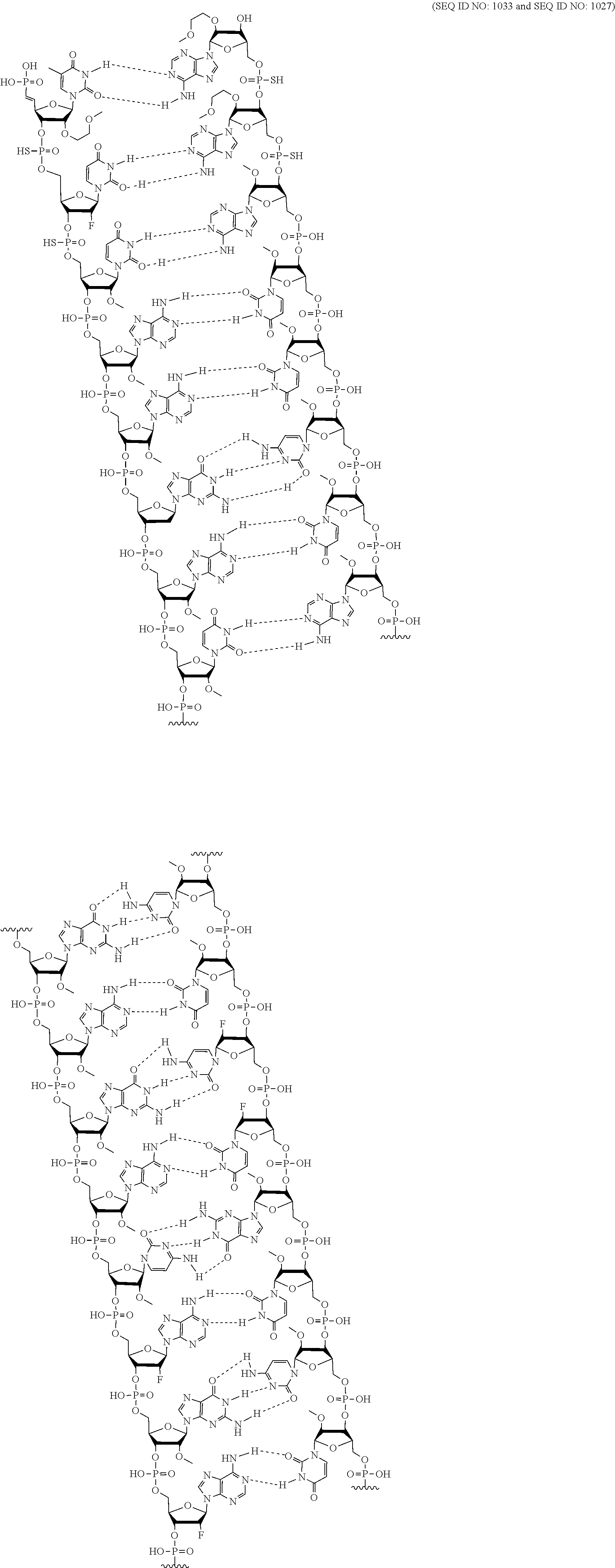

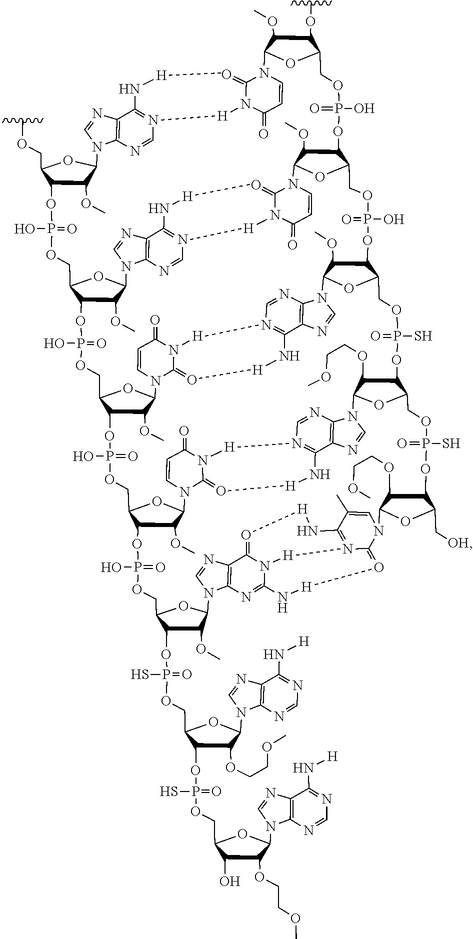

Embodiment 248. The oligomeric duplex of embodiment 247, which is the sodium salt or potassium salt.

Embodiment 249. An oligomeric duplex according to the following chemical structure:

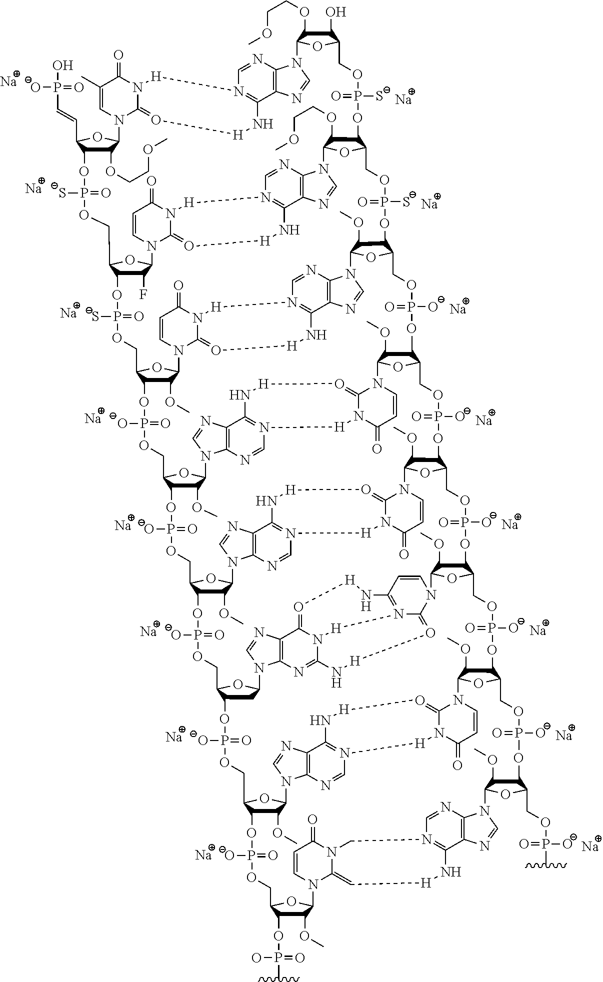

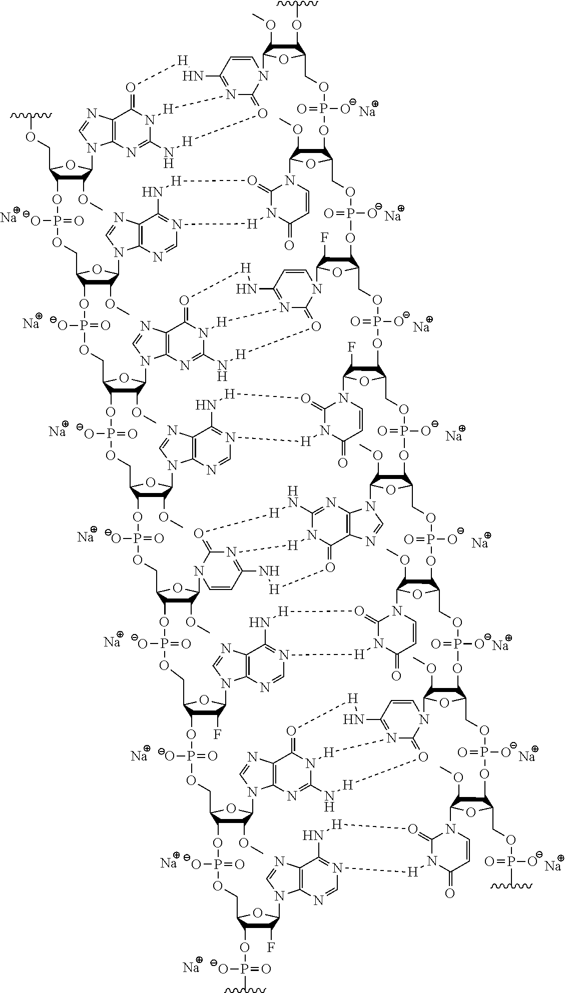

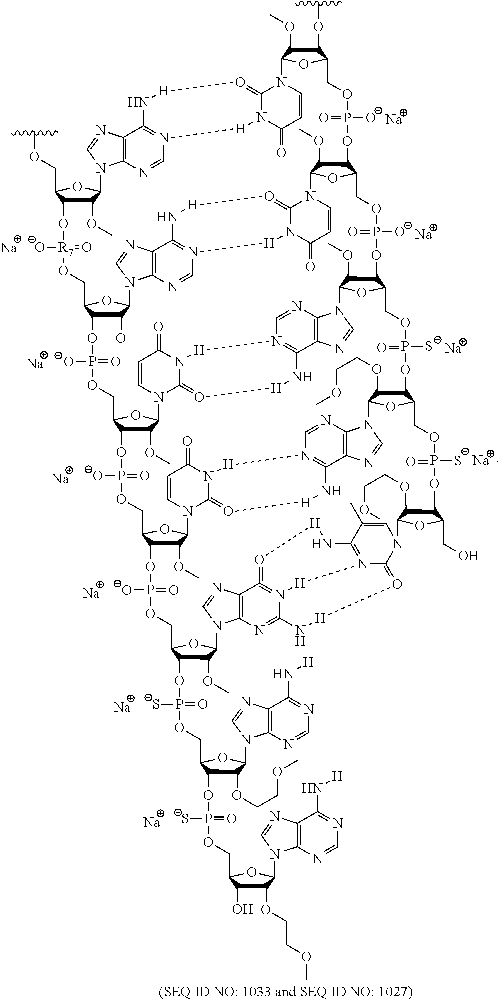

Embodiment 250. An oligomeric duplex according to the following chemical structure:

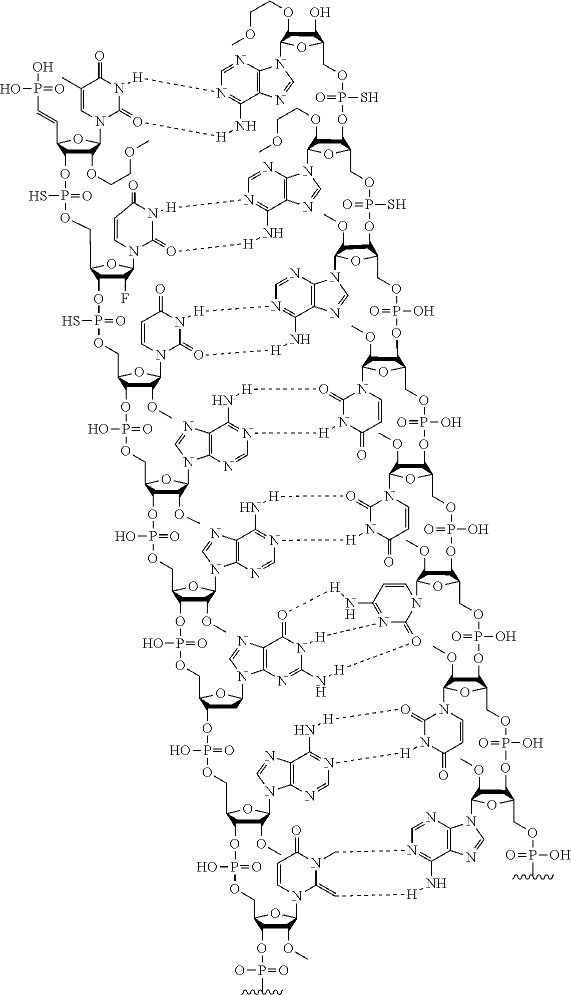

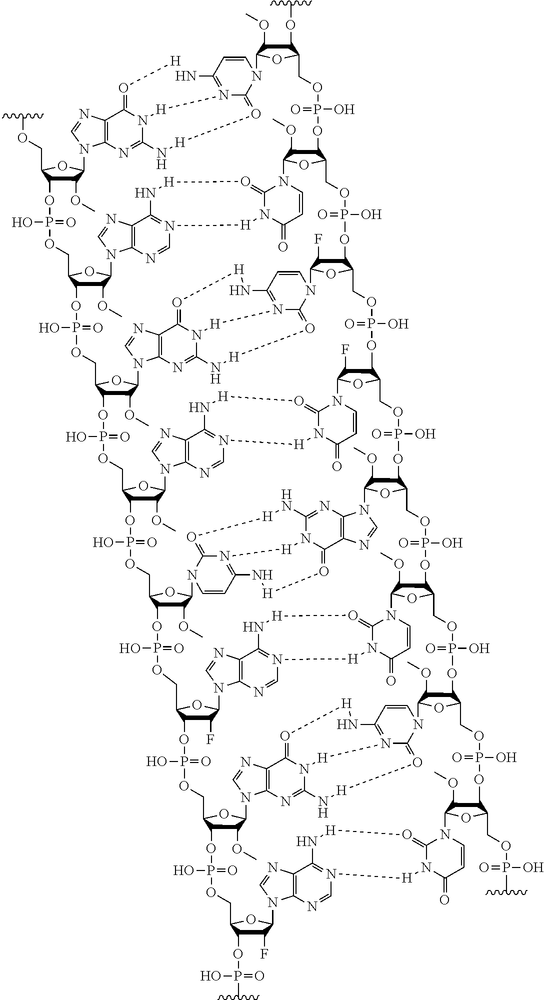

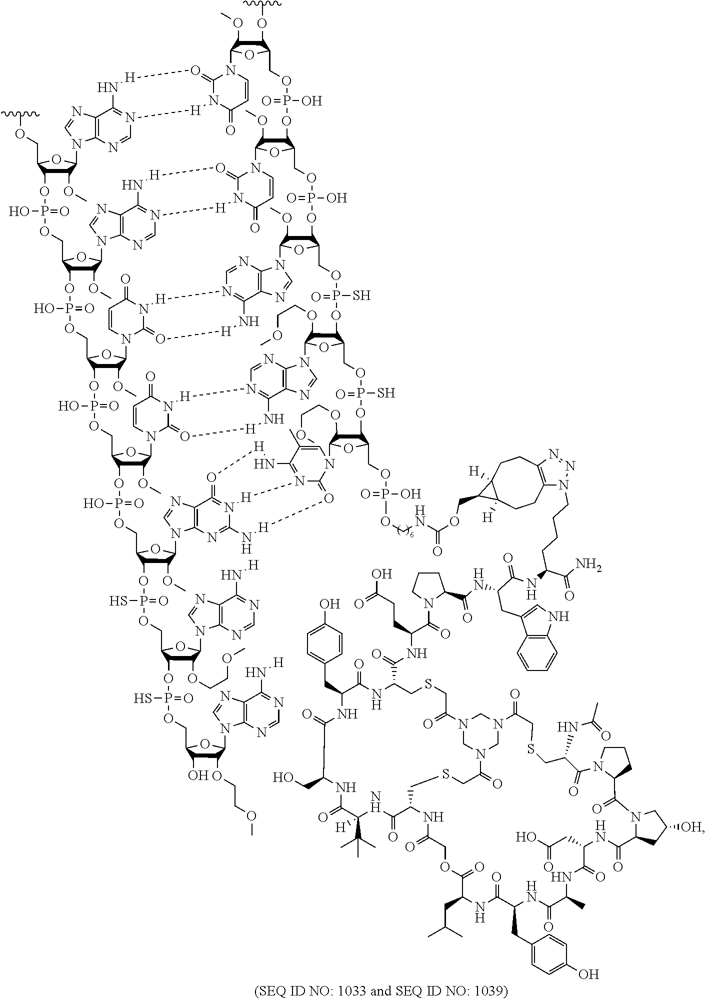

Embodiment 251. The oligomeric duplex of embodiment 250, which is the sodium salt or potassium salt.

Embodiment 252. An oligomeric duplex according to the following chemical structure:

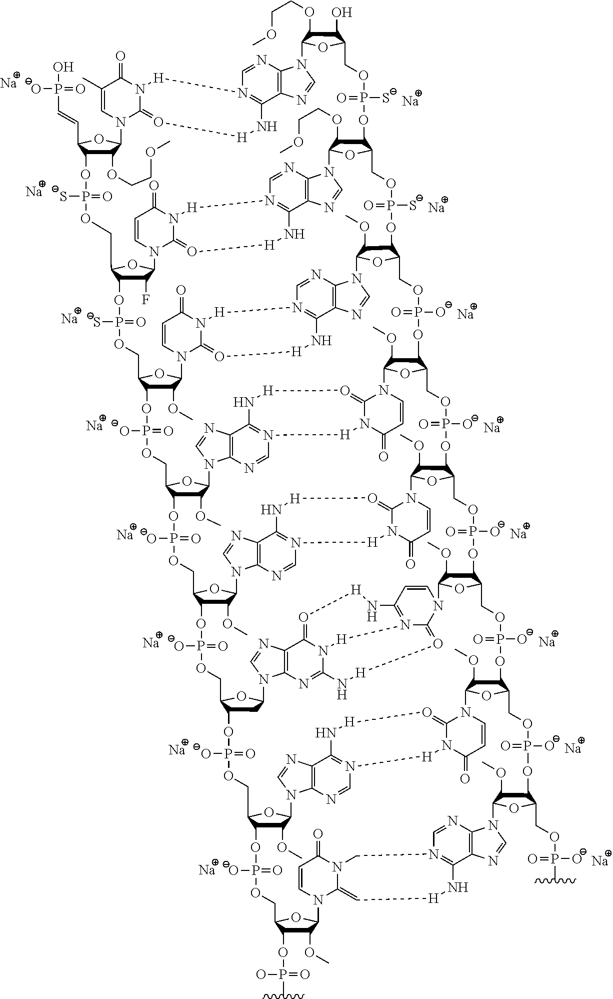

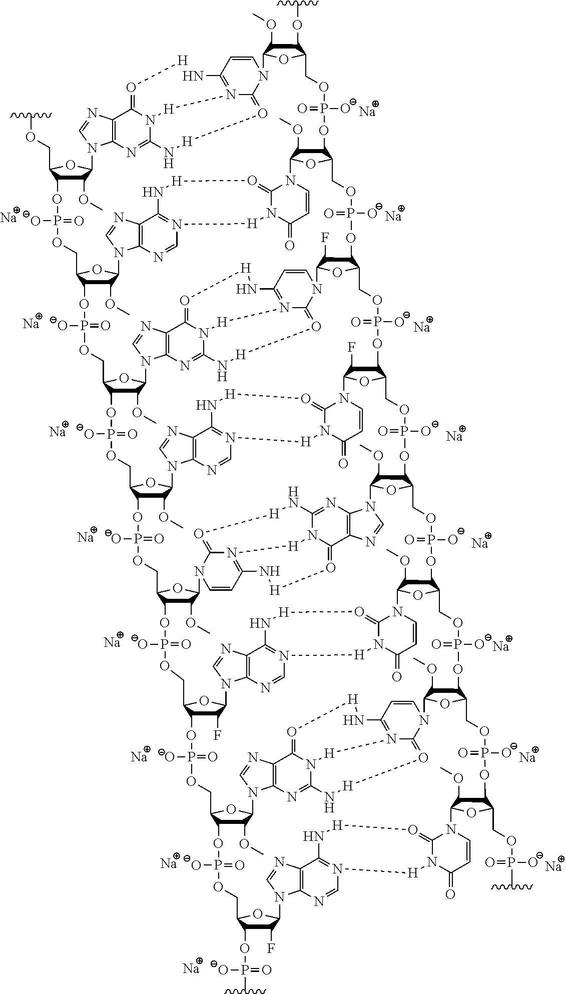

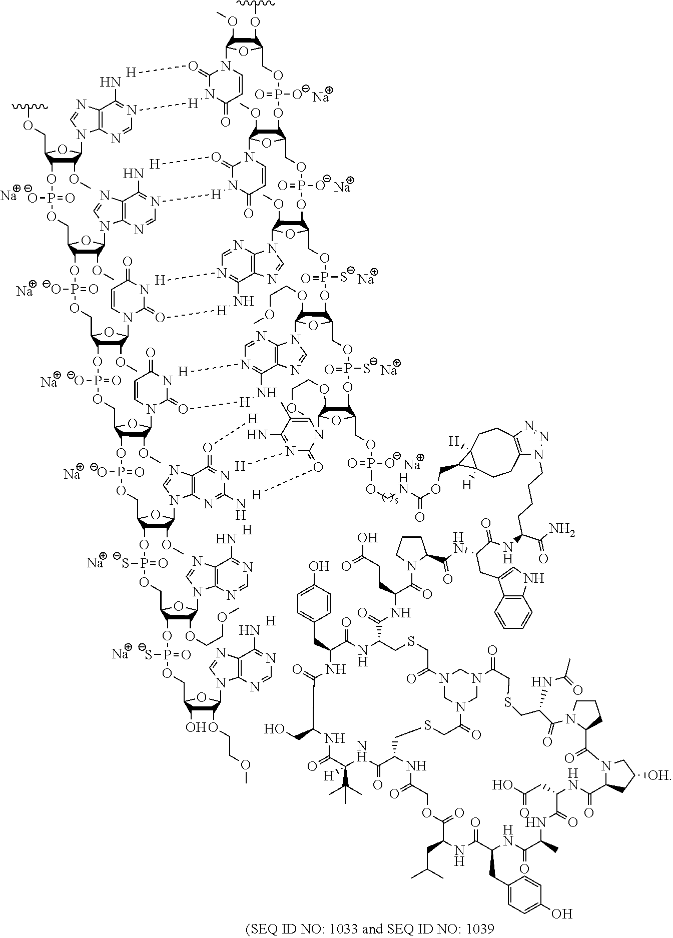

Embodiment 253. An oligomeric duplex according to the following chemical structure:

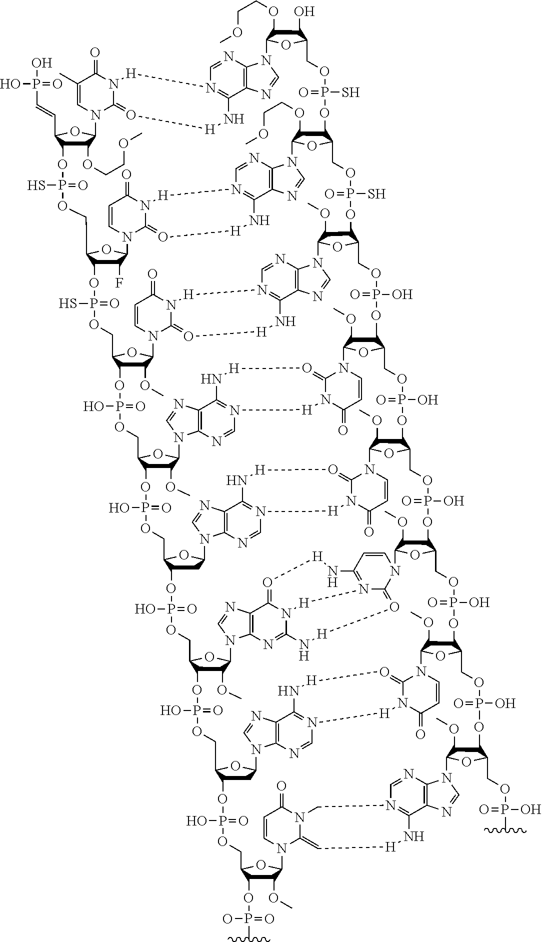

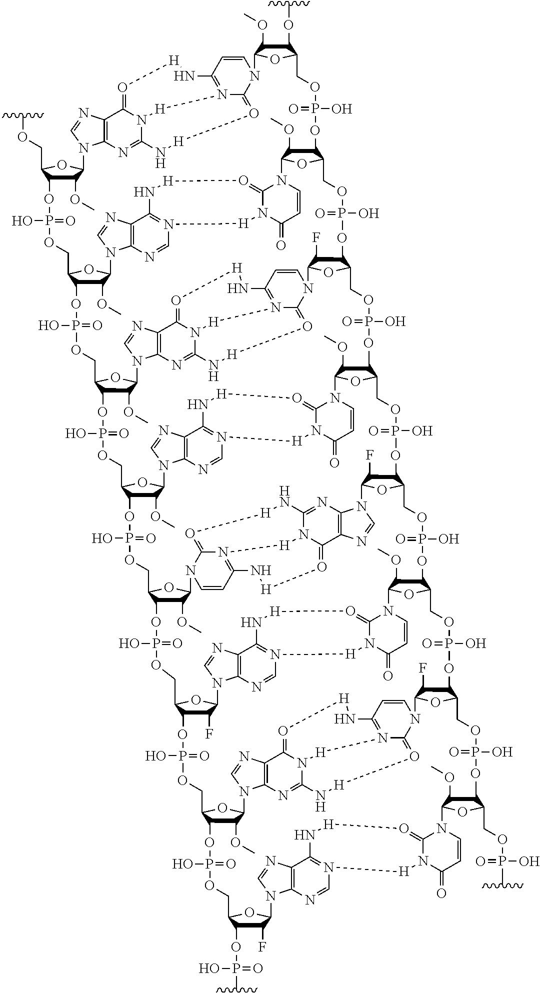

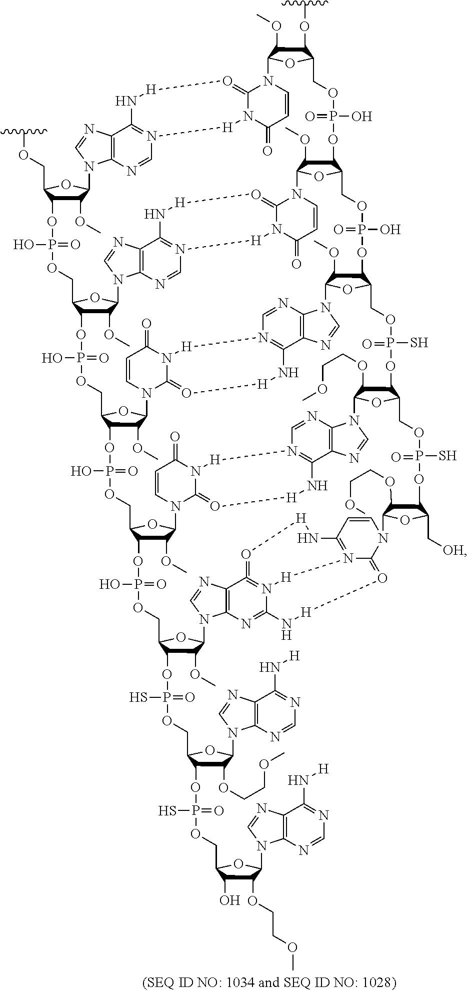

or a salt thereof.

Embodiment 254. The oligomeric duplex of embodiment 253, which is the sodium salt or potassium salt.

Embodiment 255. An oligomeric duplex according to the following chemical structure:

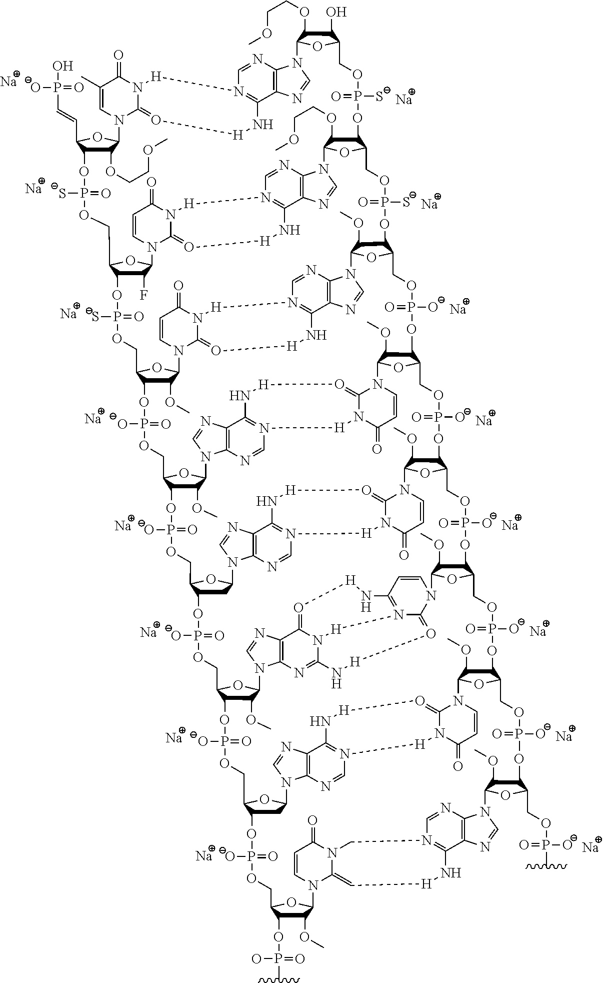

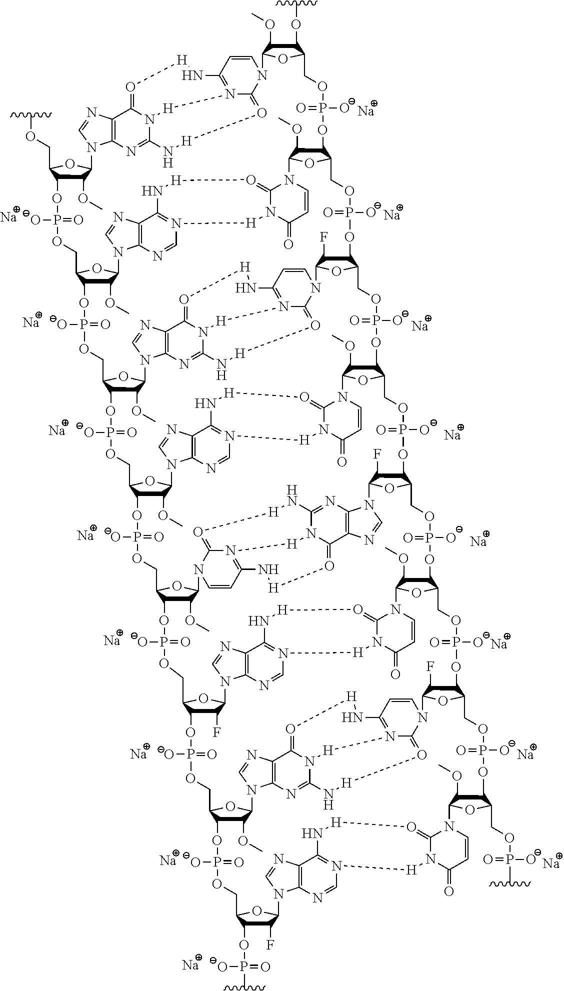

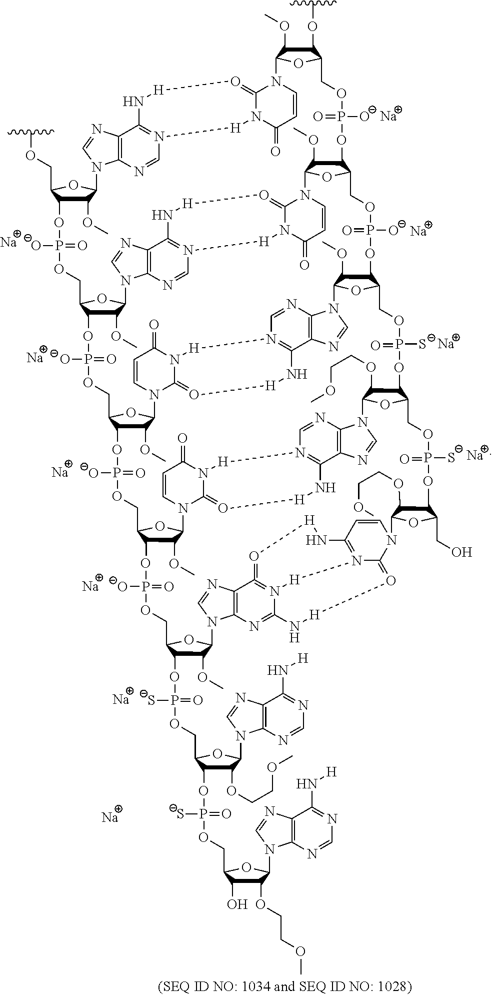

Embodiment 256. An oligomeric duplex according to the following chemical structure:

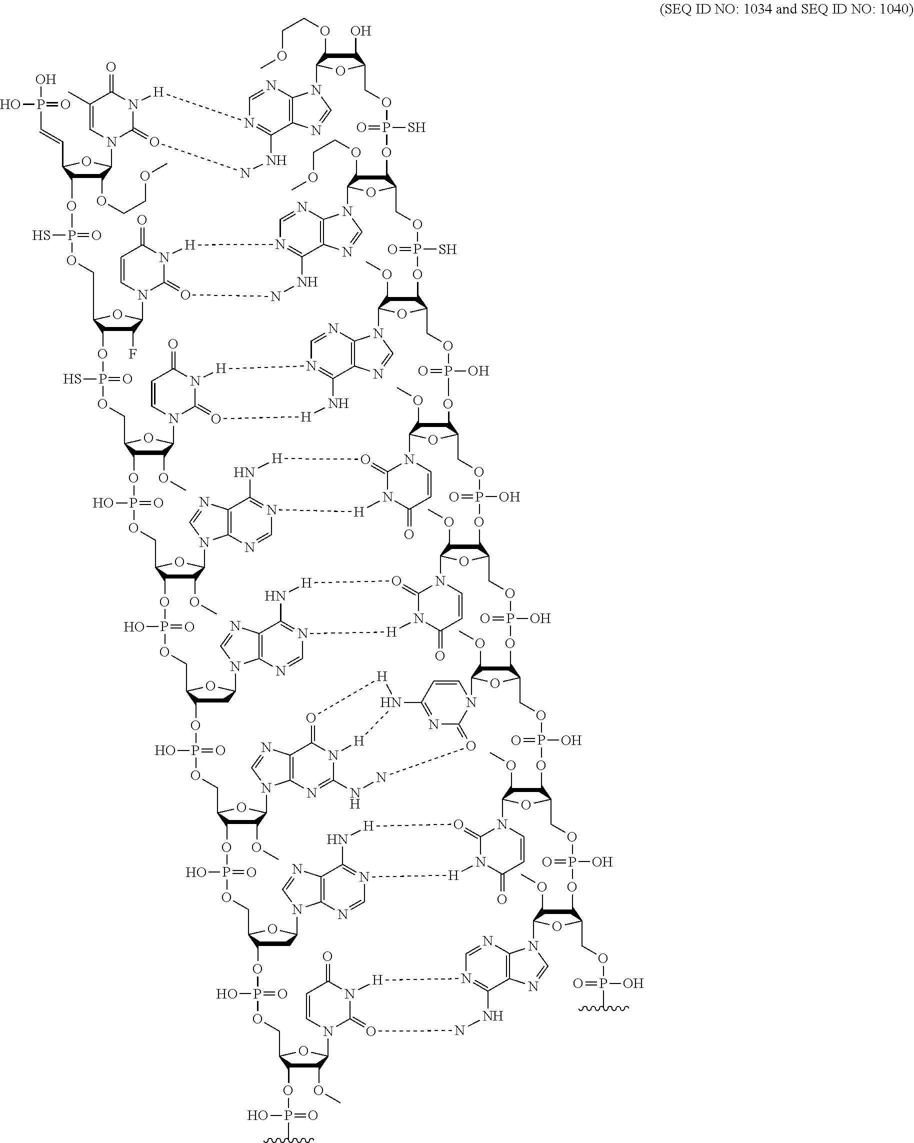

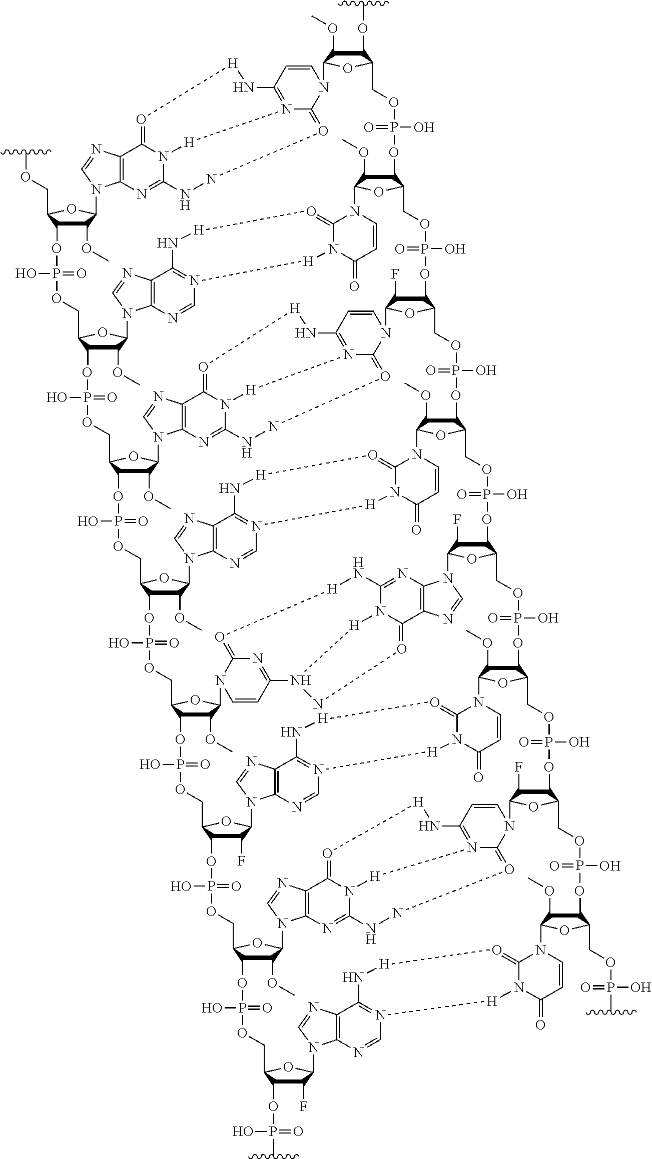

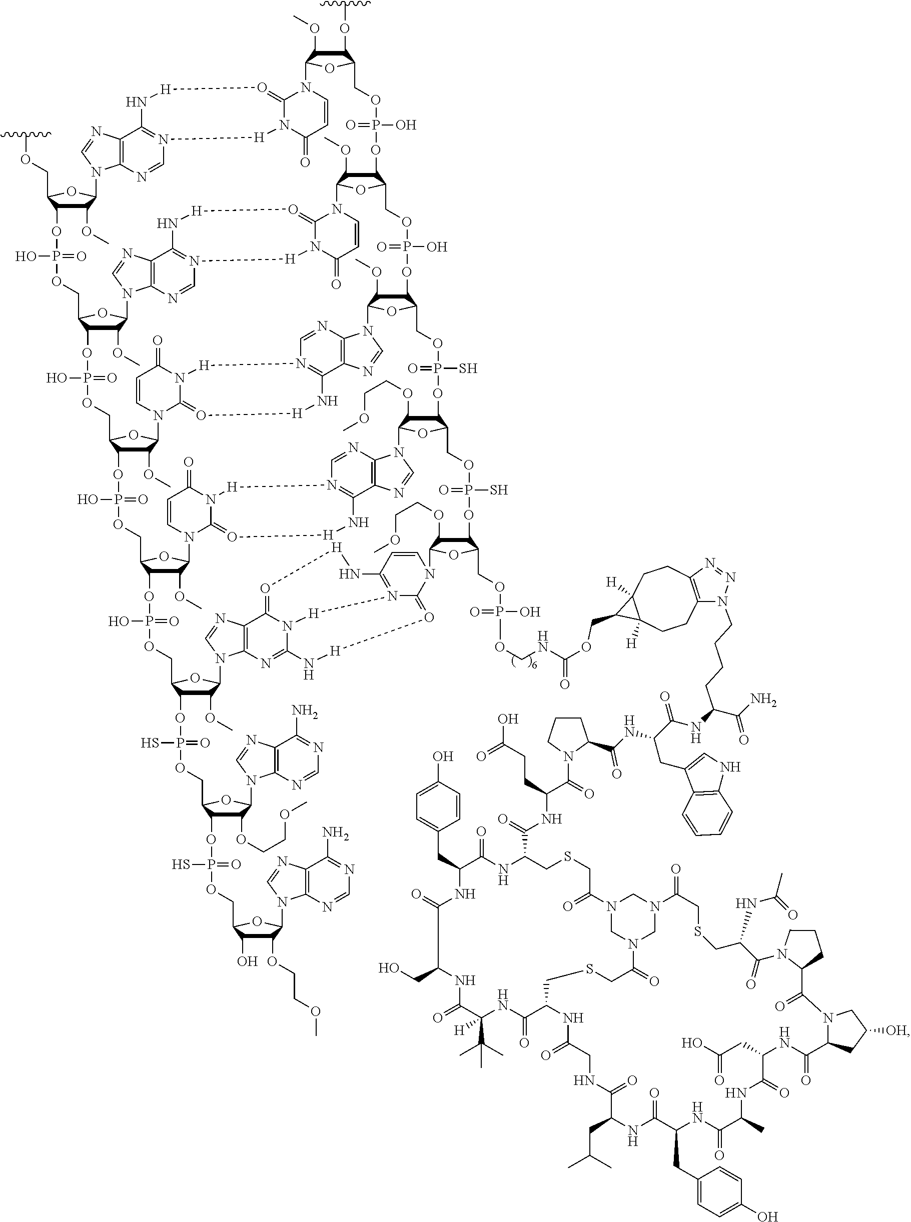

or a salt thereof.

Embodiment 257. The oligomeric duplex of embodiment 256, which is the sodium salt or potassium salt.

Embodiment 258. An oligomeric duplex according to the following chemical structure:

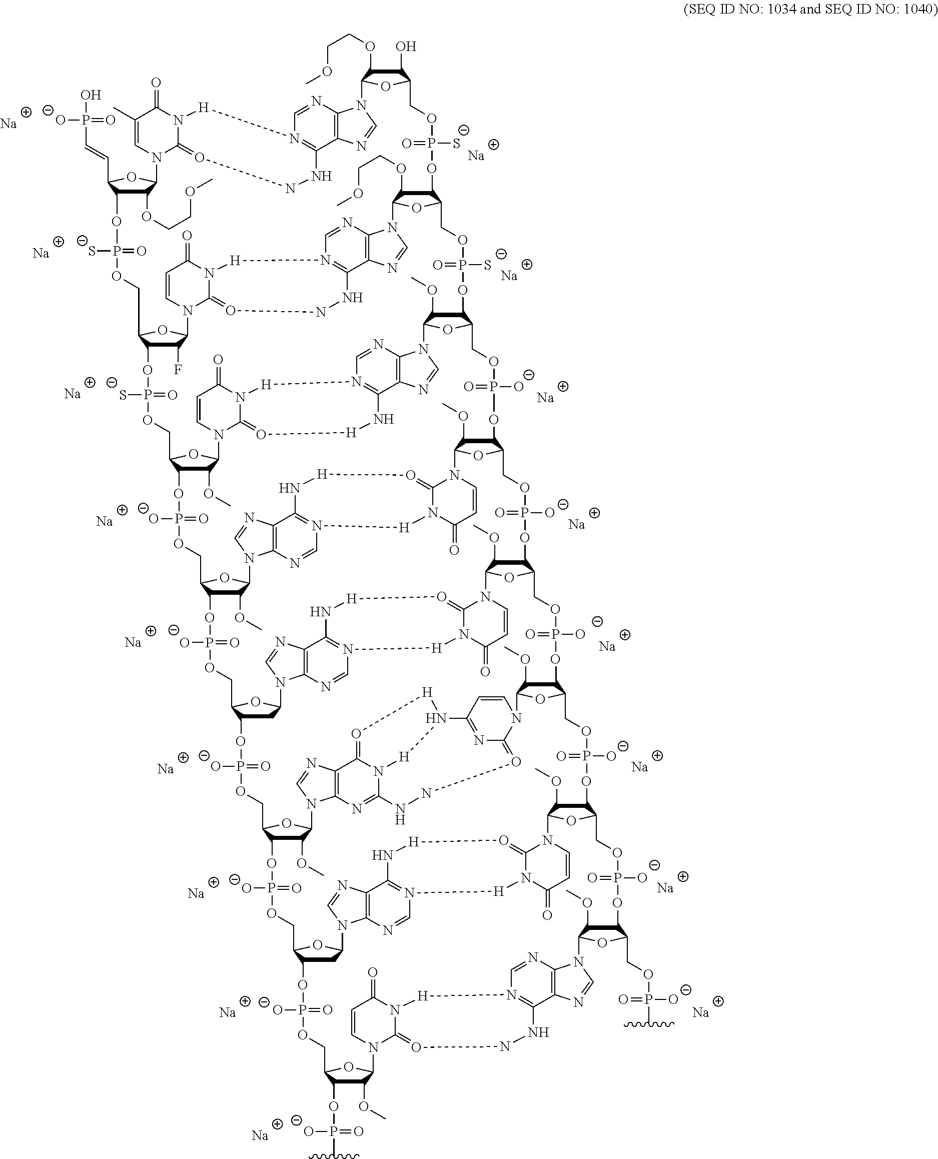

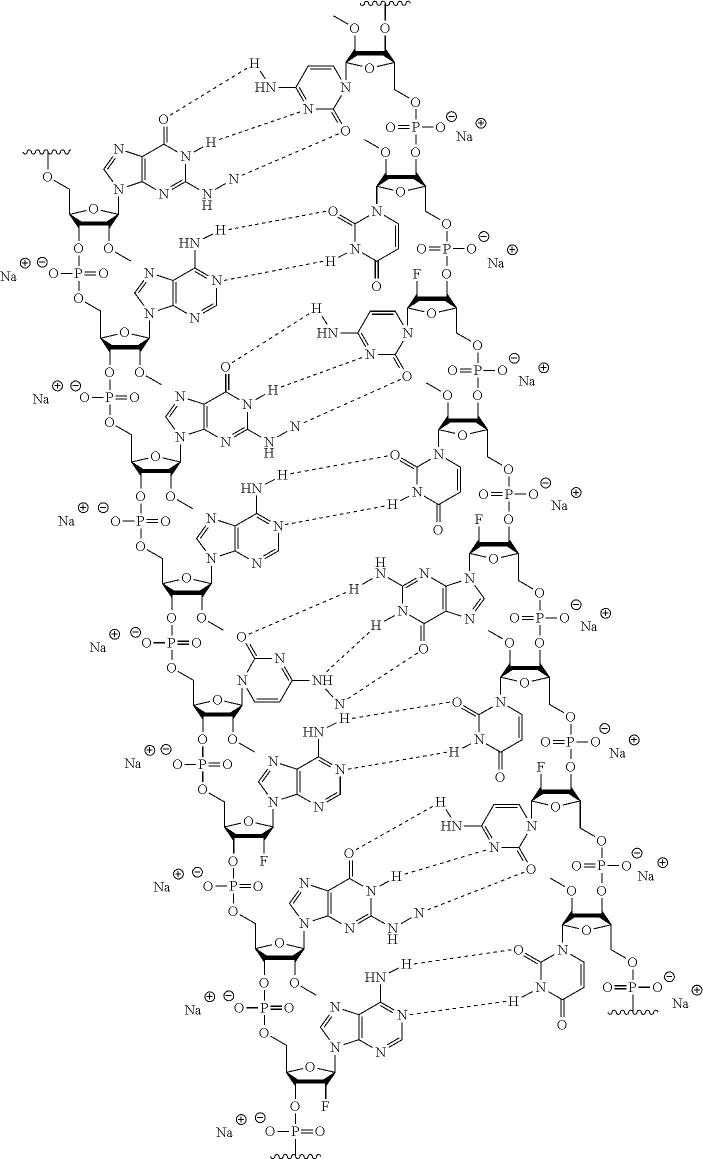

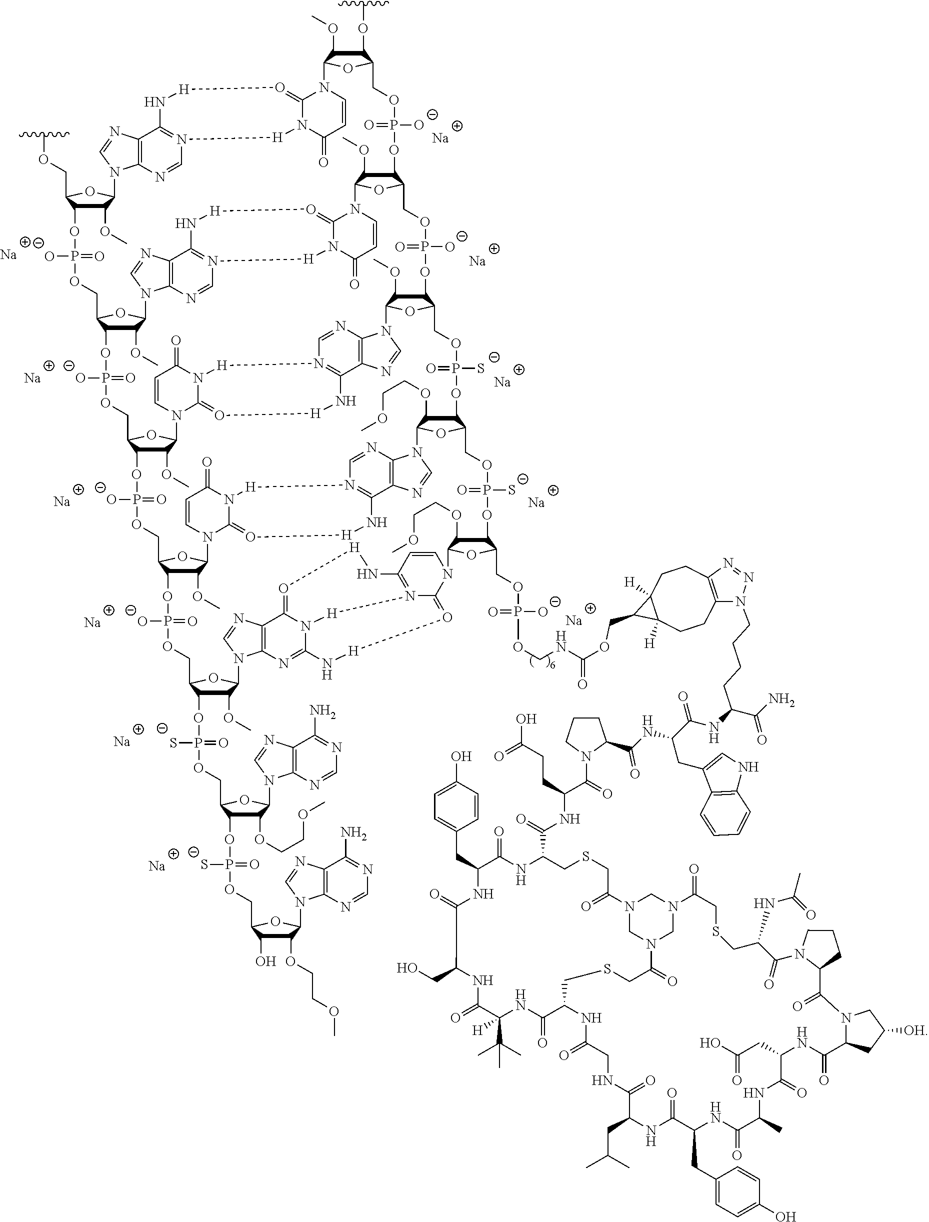

Embodiment 259. An oligomeric duplex according to the following chemical structure:

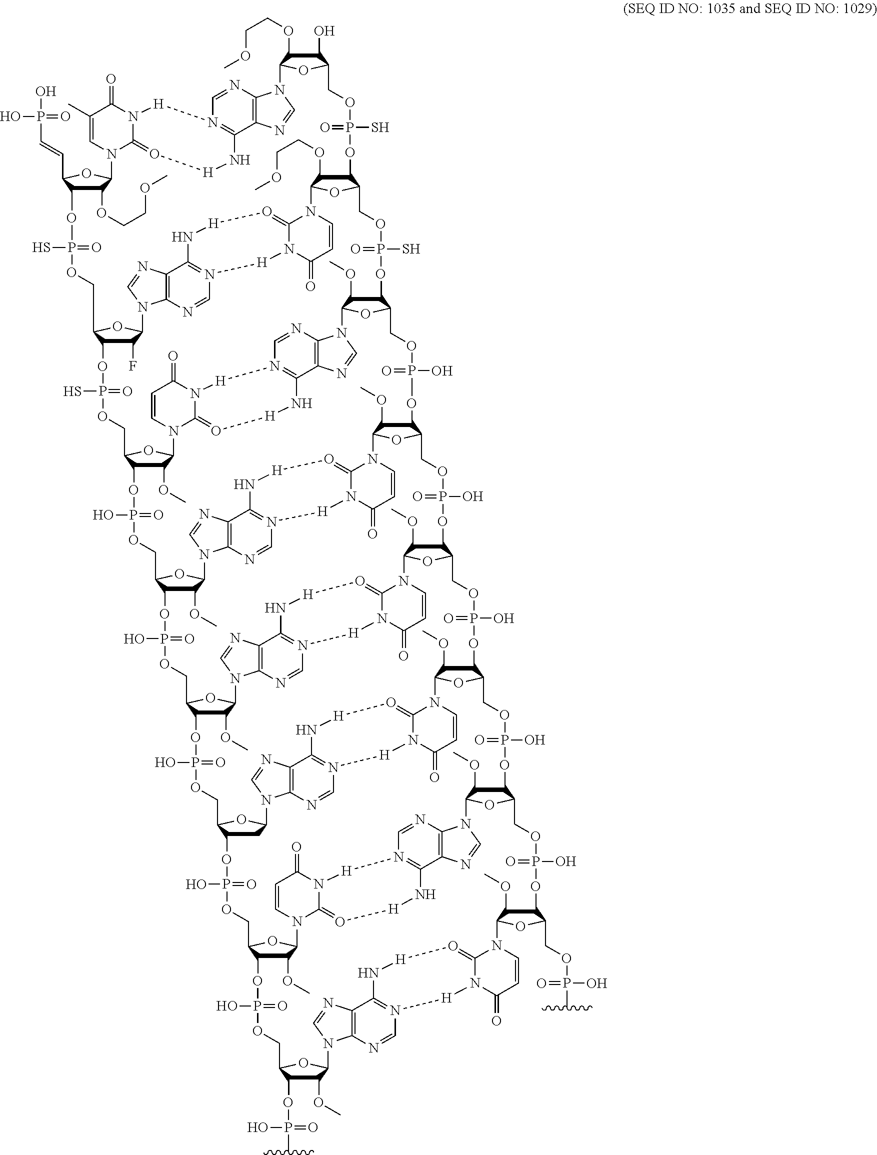

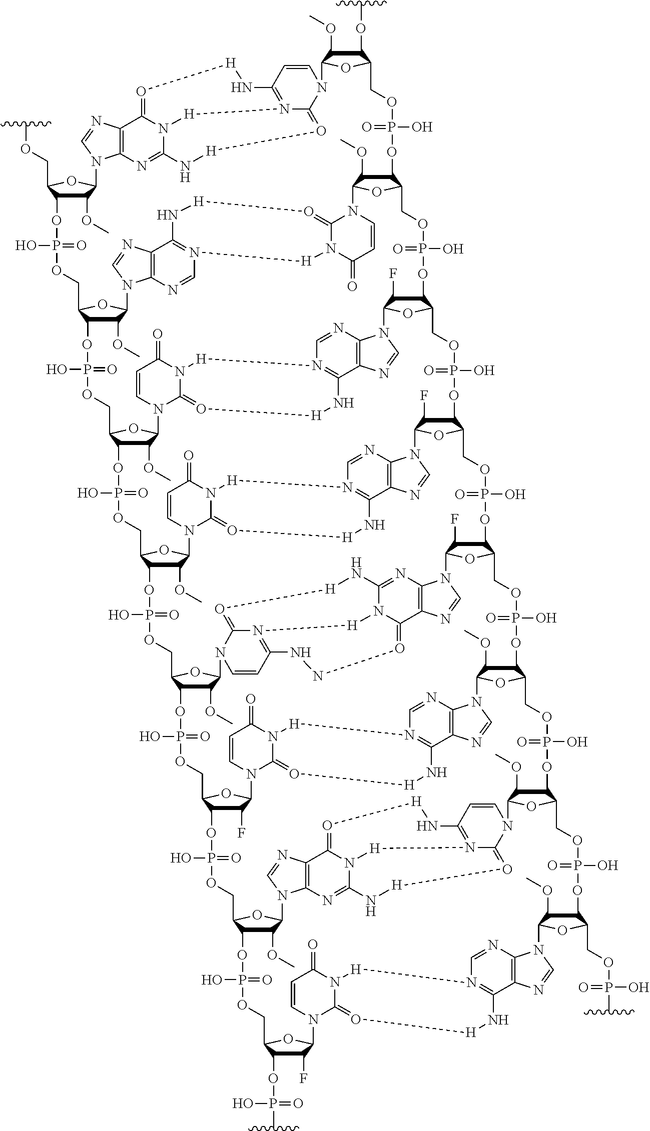

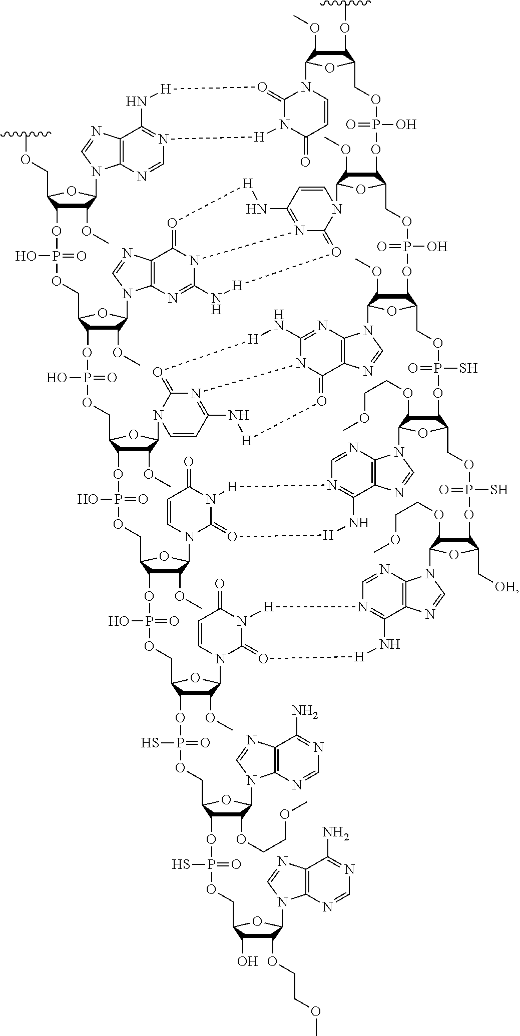

or a salt thereof.

Embodiment 260. The oligomeric duplex of embodiment 259, which is the sodium salt or potassium salt.

Embodiment 261. An oligomeric duplex according to the following chemical structure:

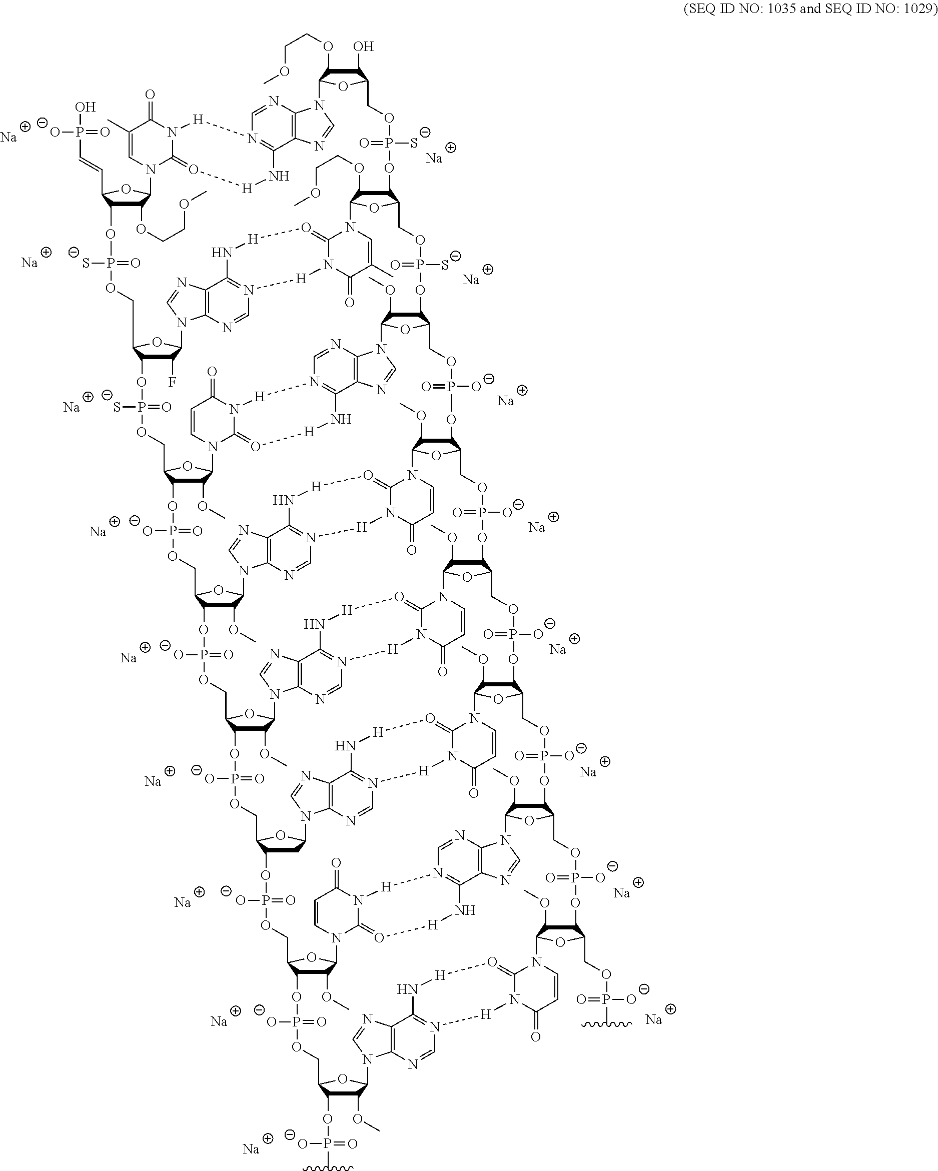

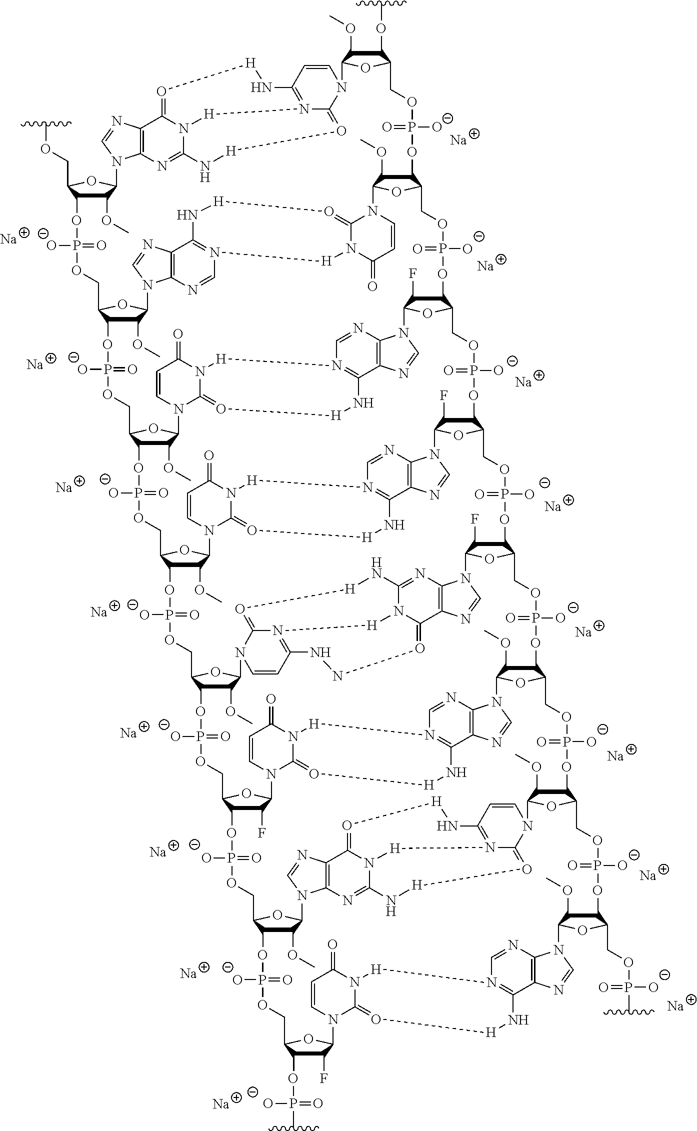

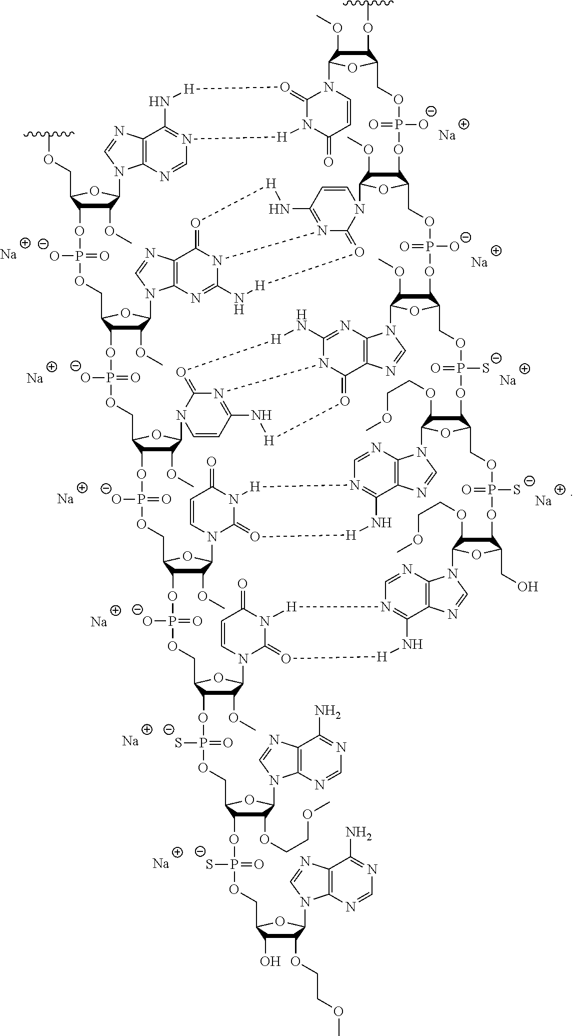

Embodiment 262. An oligomeric duplex according to the following chemical structure:

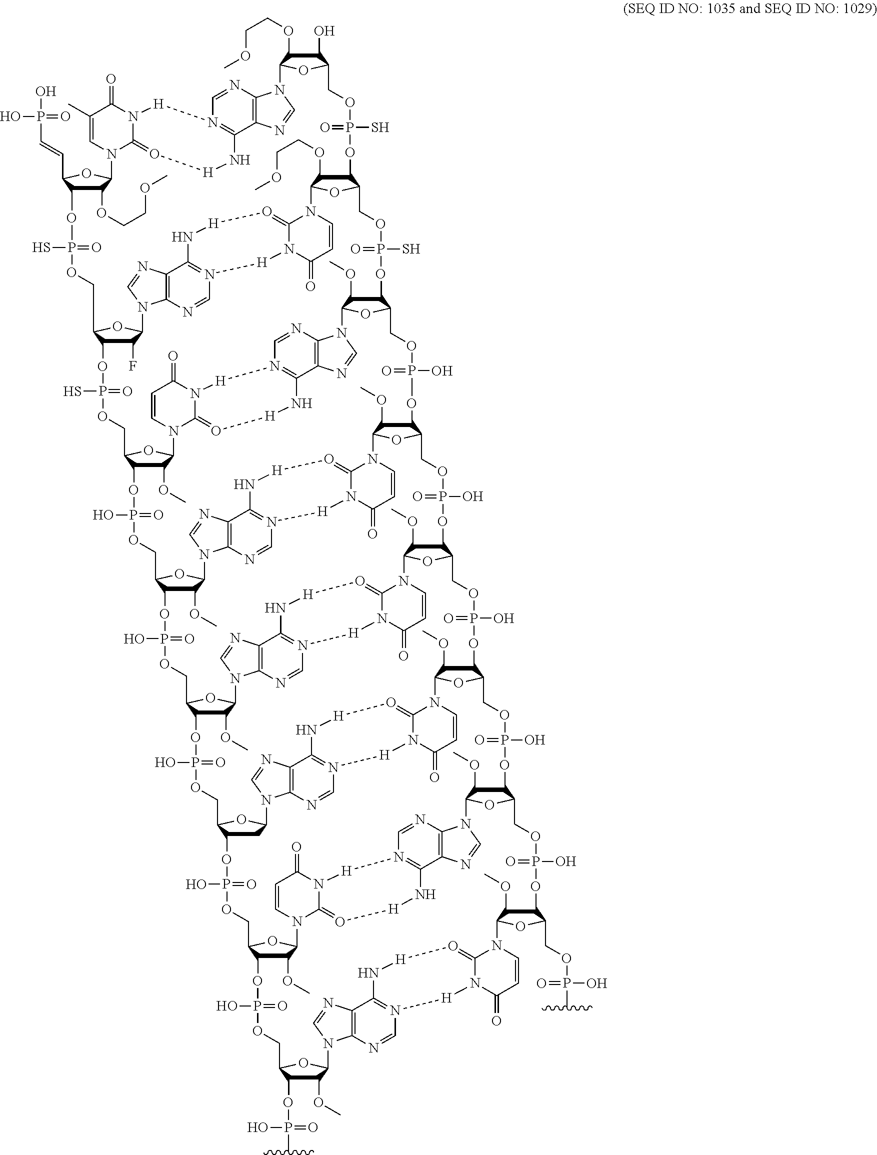

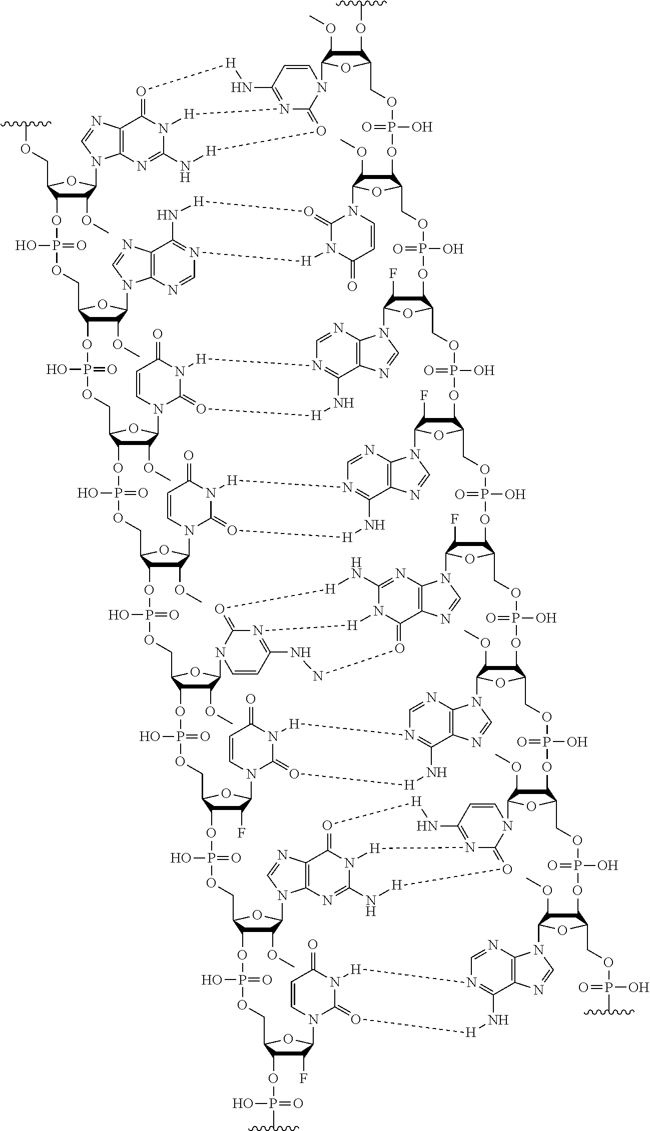

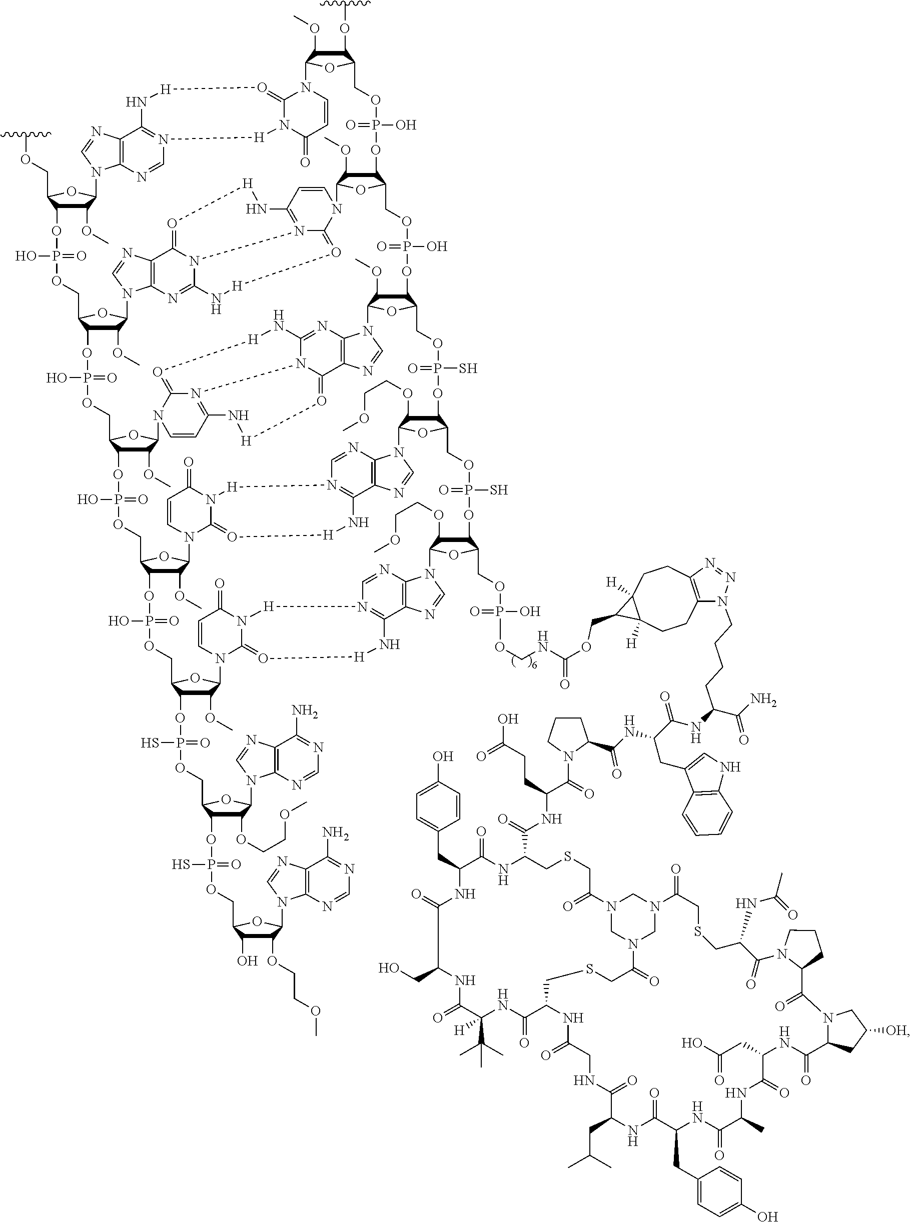

or a salt thereof.

Embodiment 263. The oligomeric duplex of embodiment 262, which is the sodium salt or potassium salt.

Embodiment 264. An oligomeric duplex according to the following chemical structure:

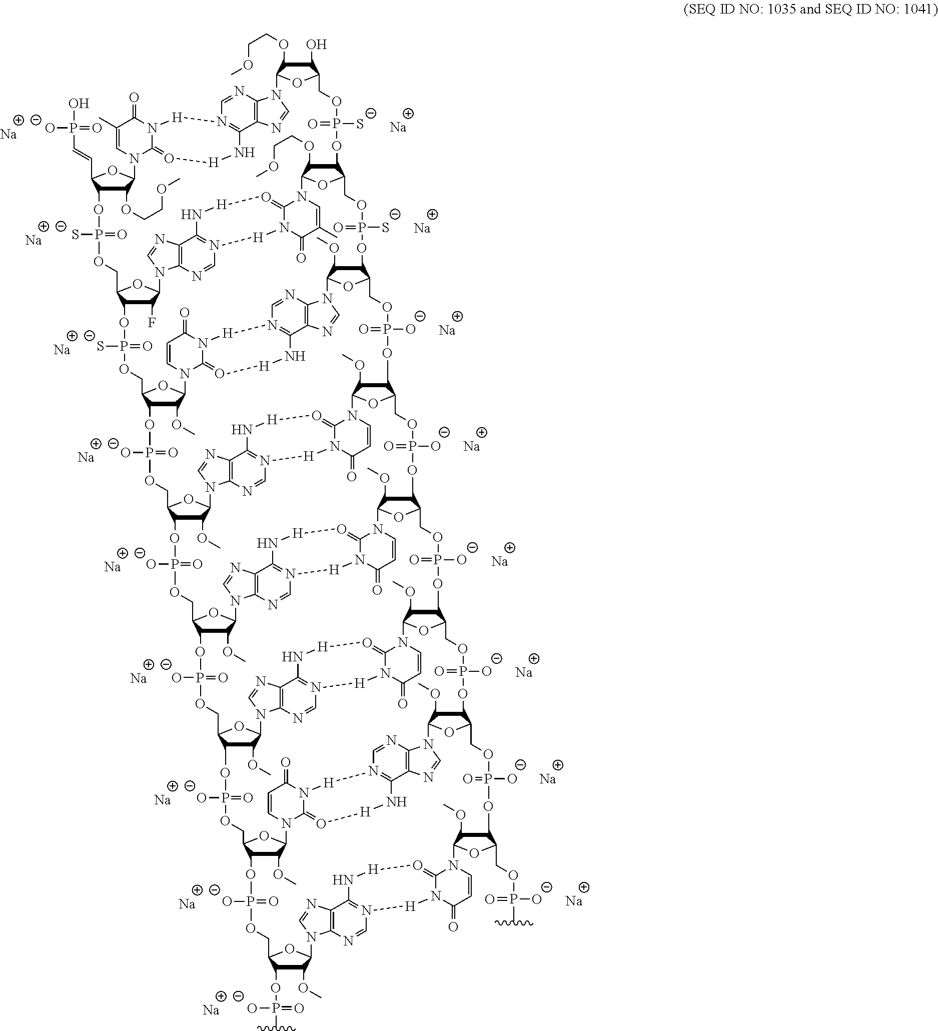

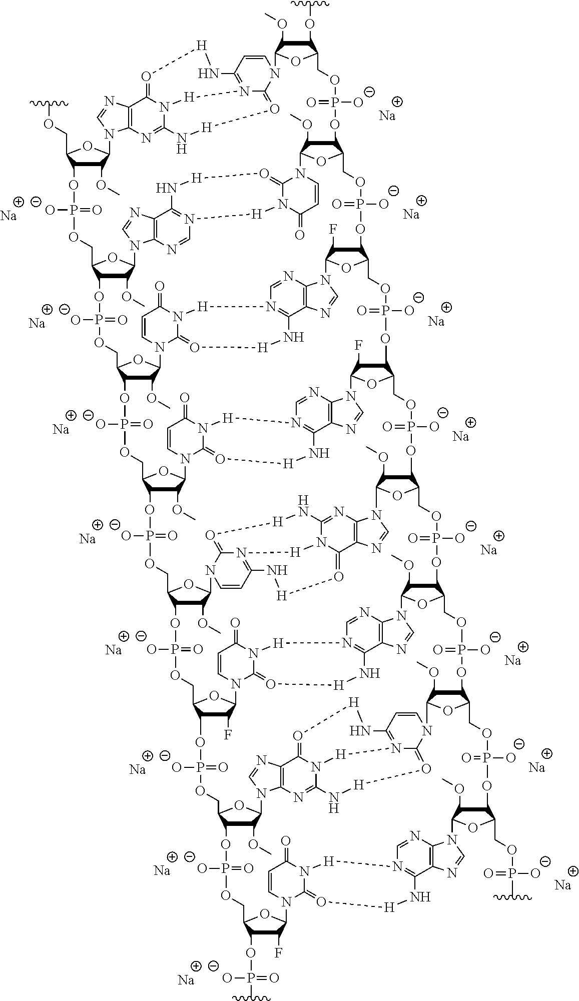

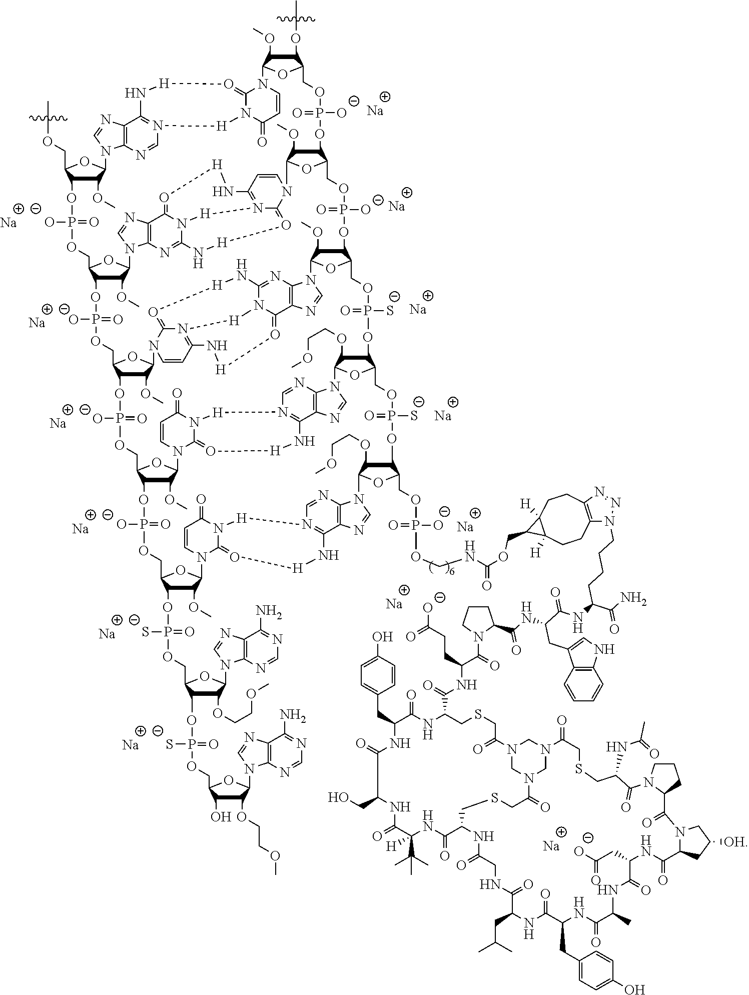

Embodiment 265. An oligomeric duplex according to the following chemical structure:

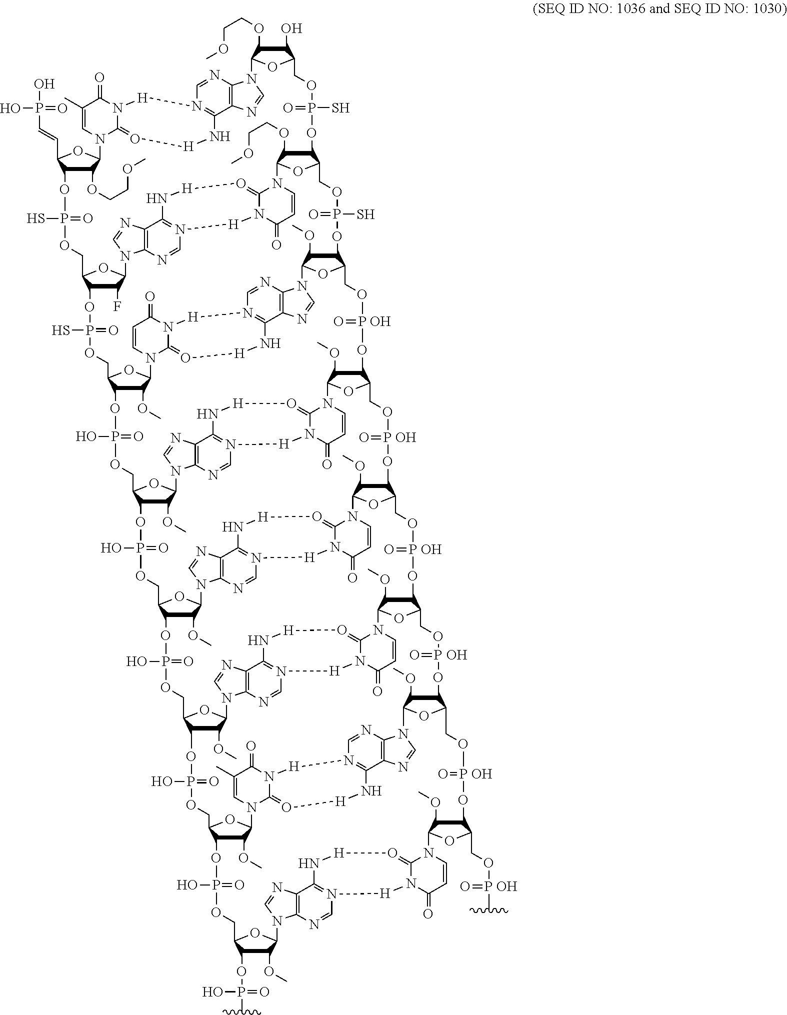

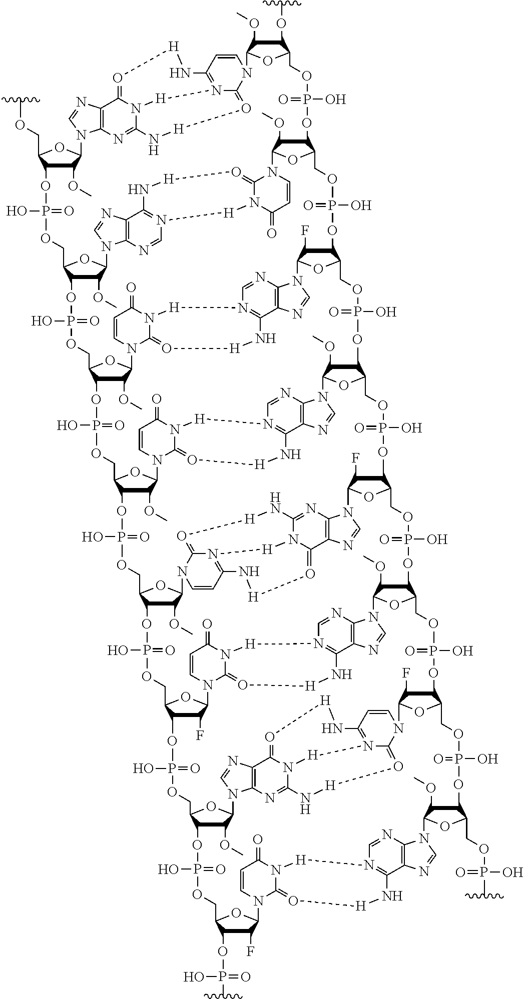

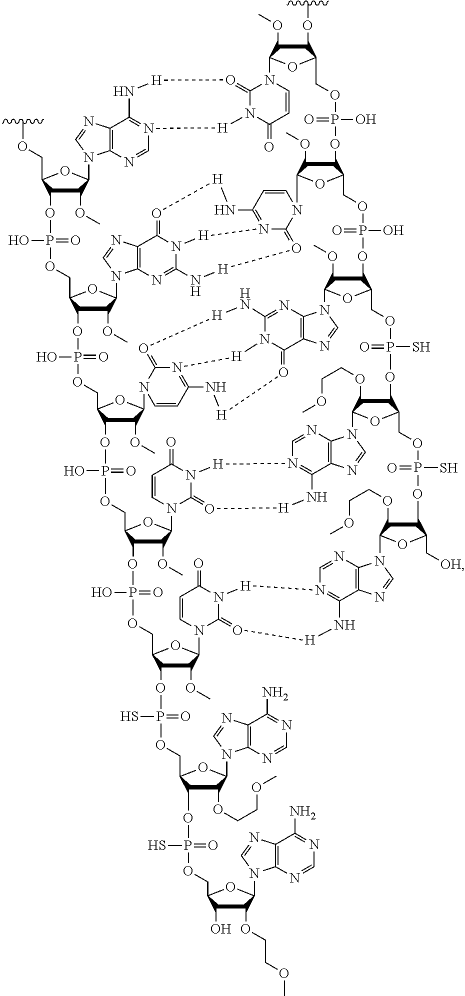

or a salt thereof.

Embodiment 266. The oligomeric duplex of embodiment 265, which is the sodium salt or potassium salt.

Embodiment 267. An oligomeric duplex according to the following chemical structure:

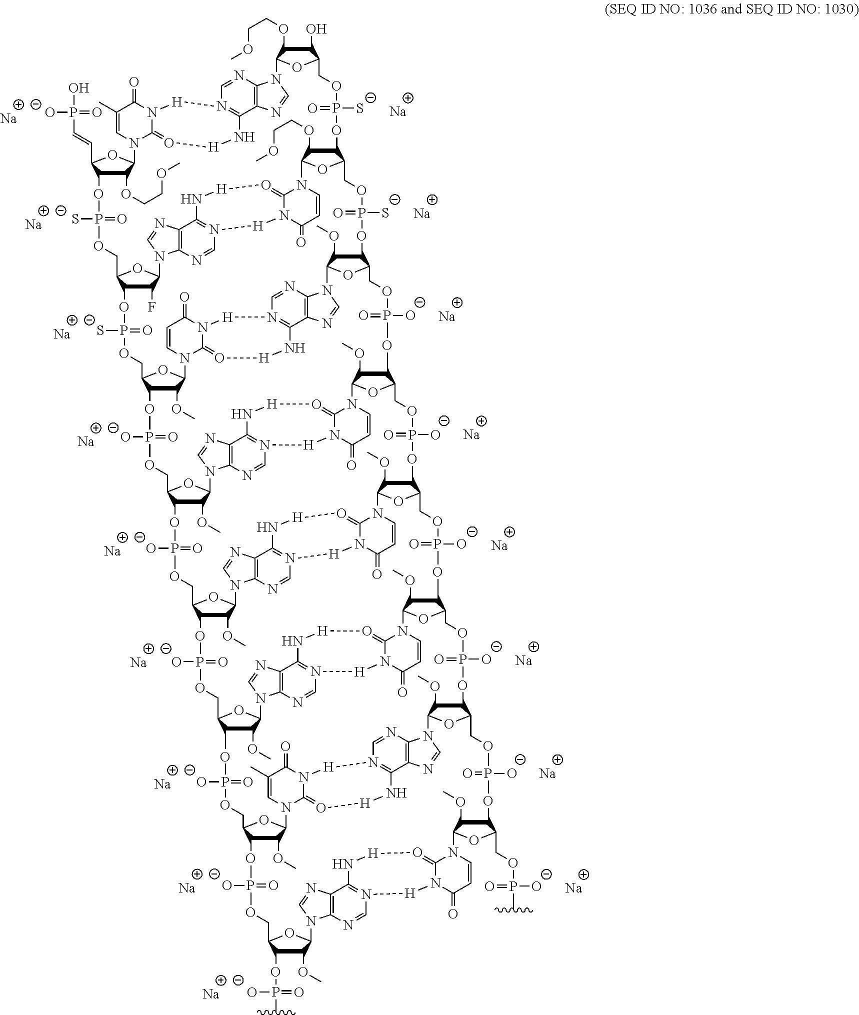

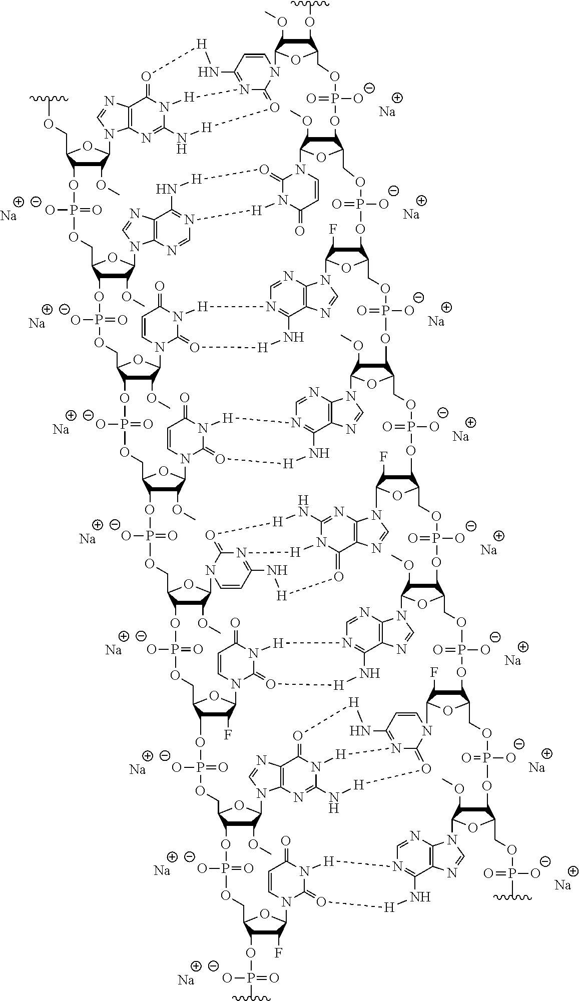

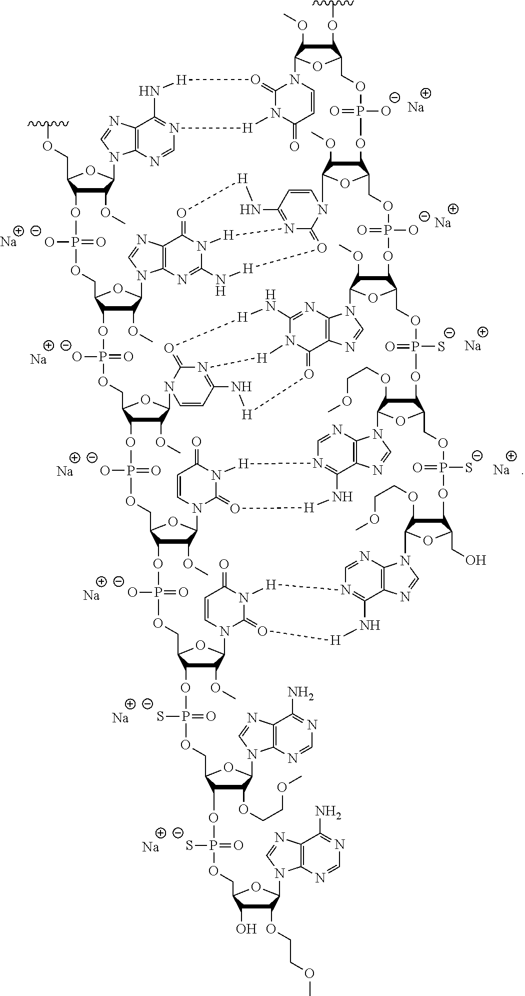

Embodiment 268. An oligomeric duplex according to the following chemical structure:

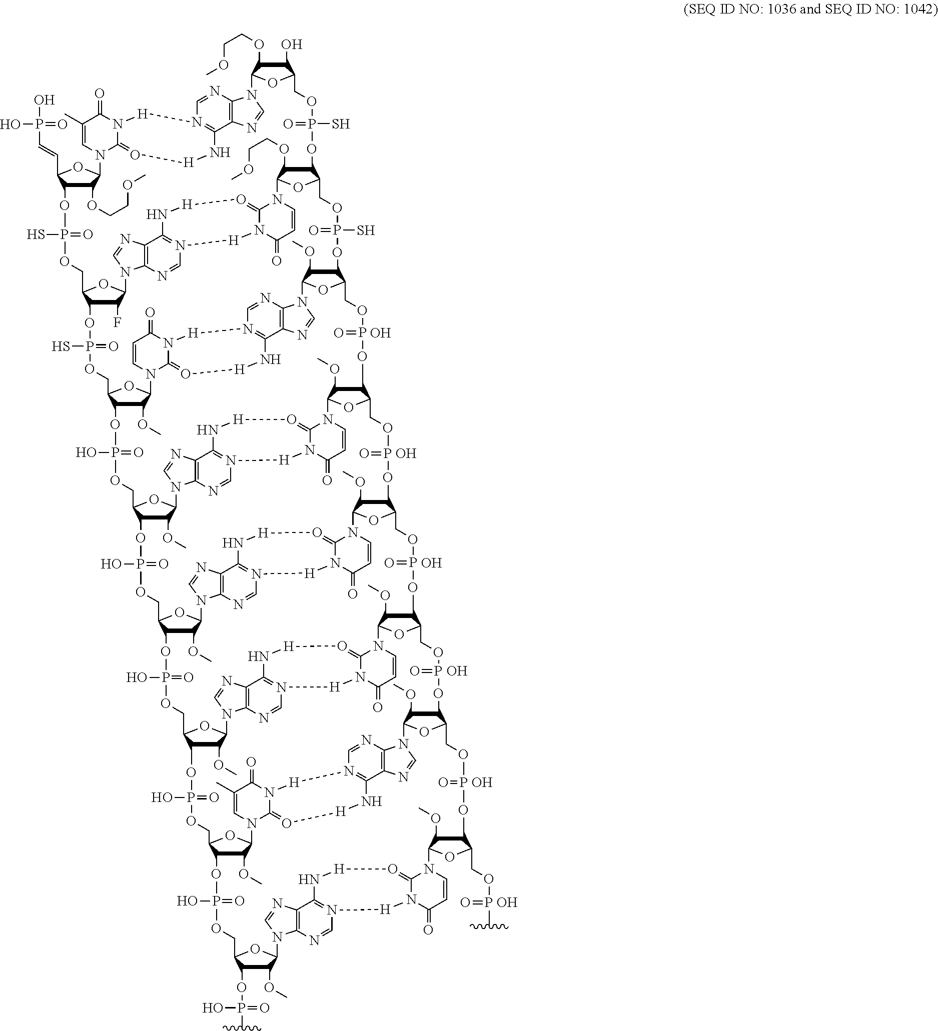

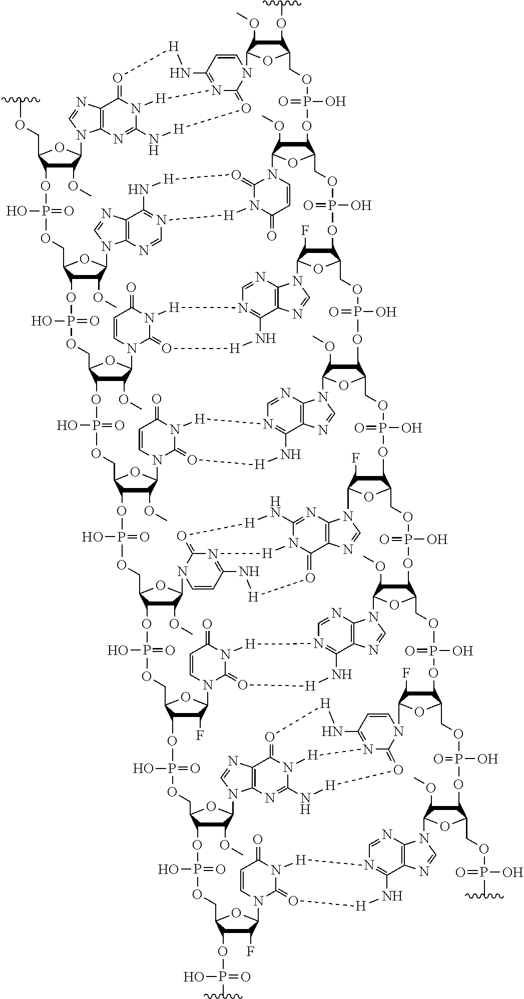

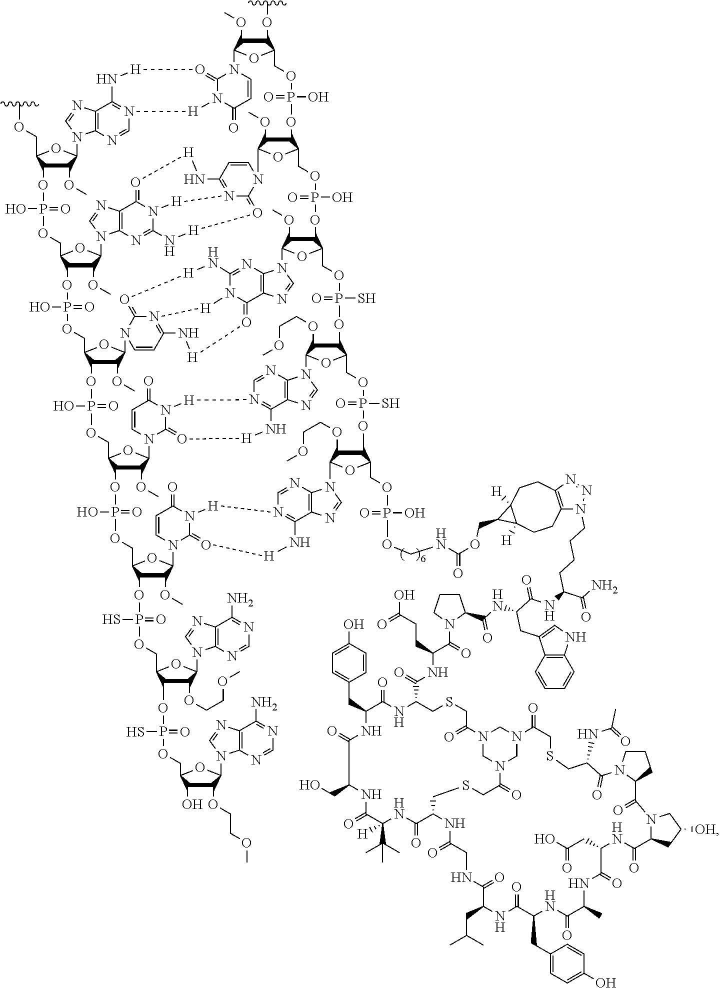

or a salt thereof.

Embodiment 269. The oligomeric duplex of embodiment 268, which is the sodium salt or potassium salt.

Embodiment 270. An oligomeric duplex according to the following chemical structure:

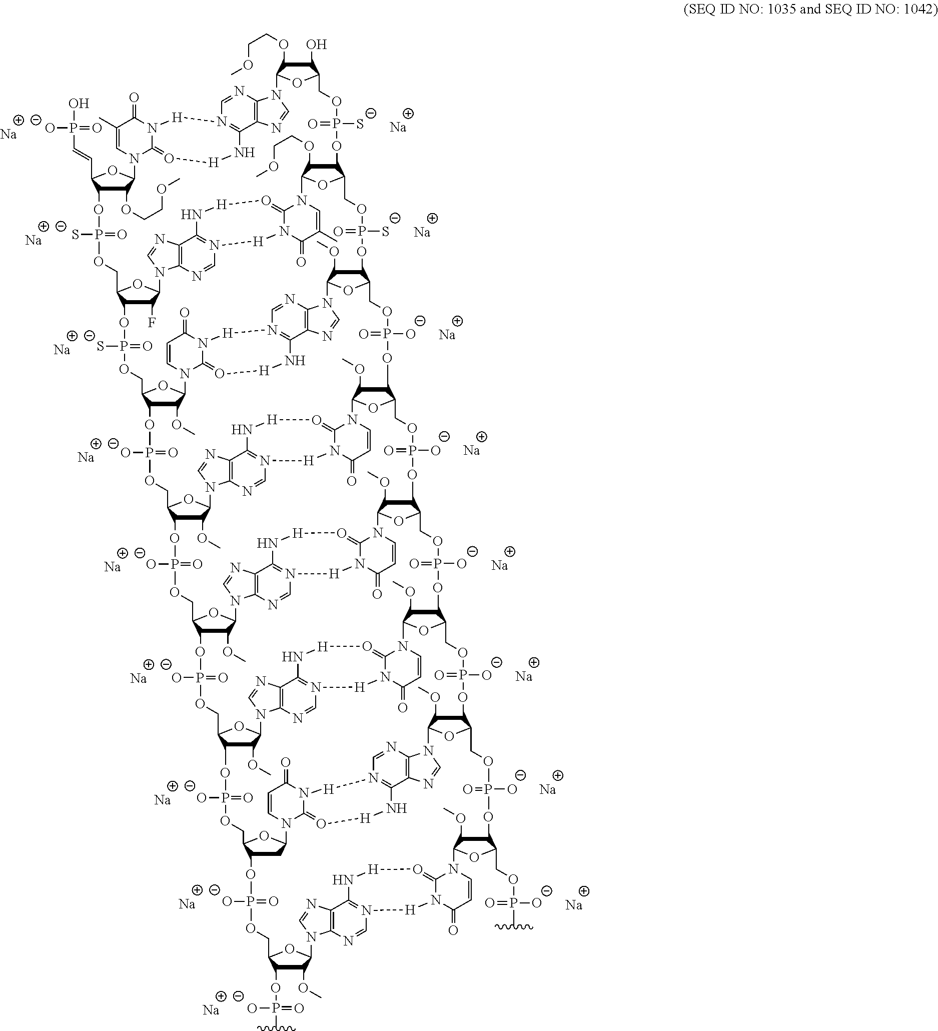

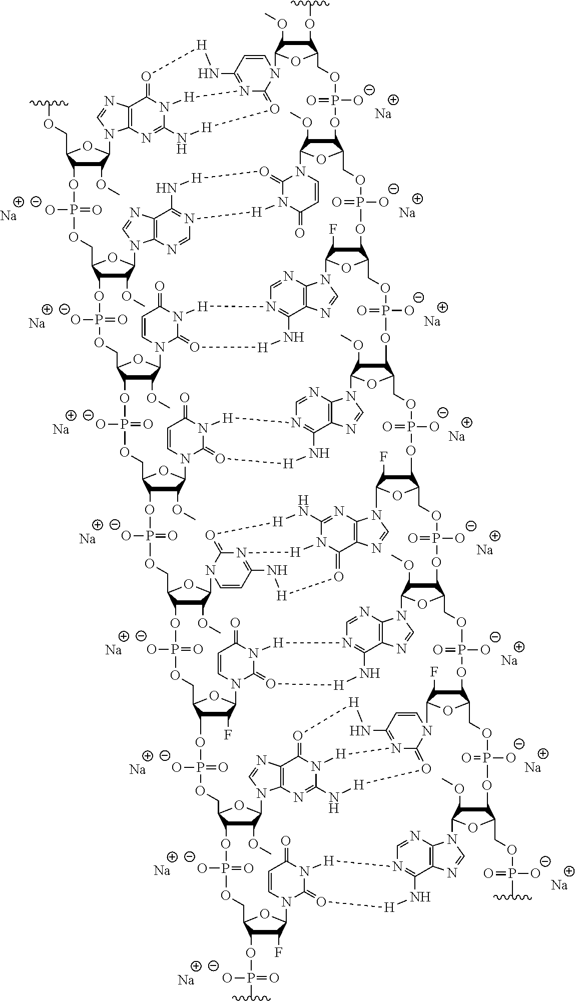

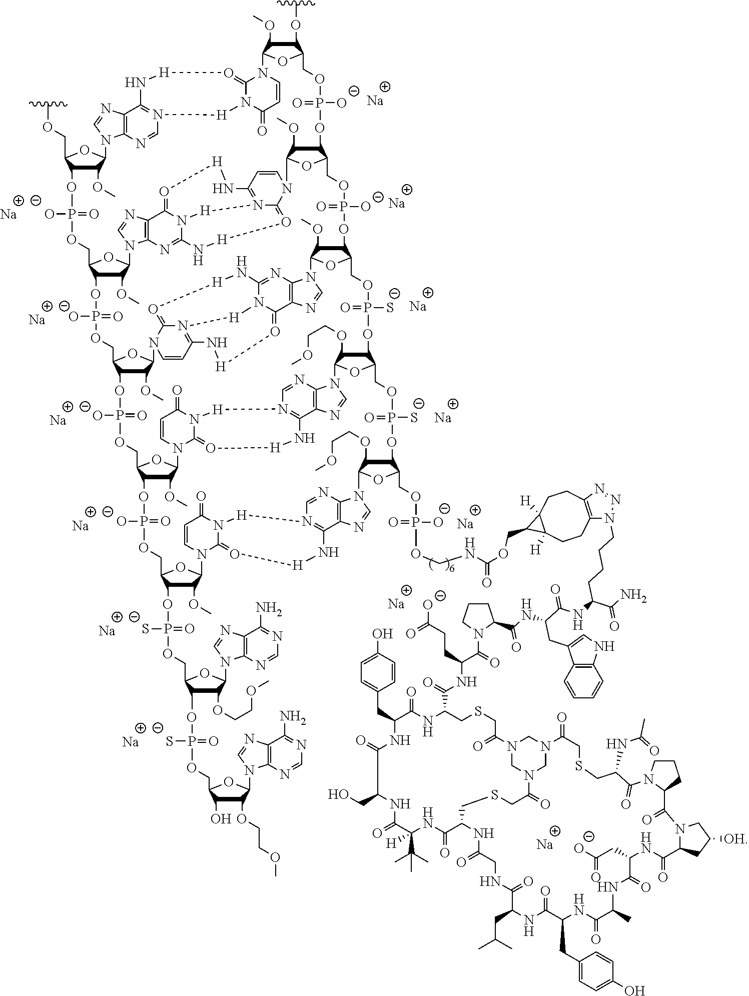

Embodiment 271. An oligomeric duplex according to the following chemical structure:

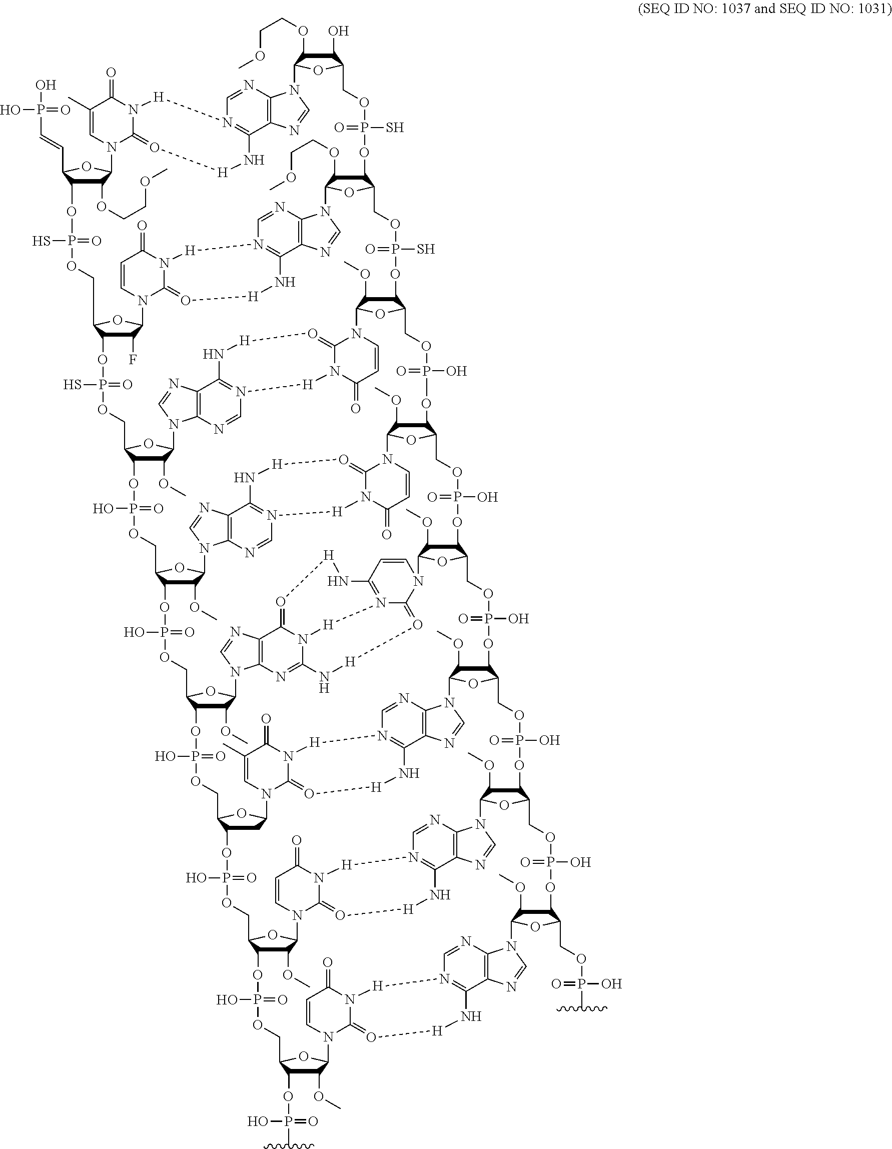

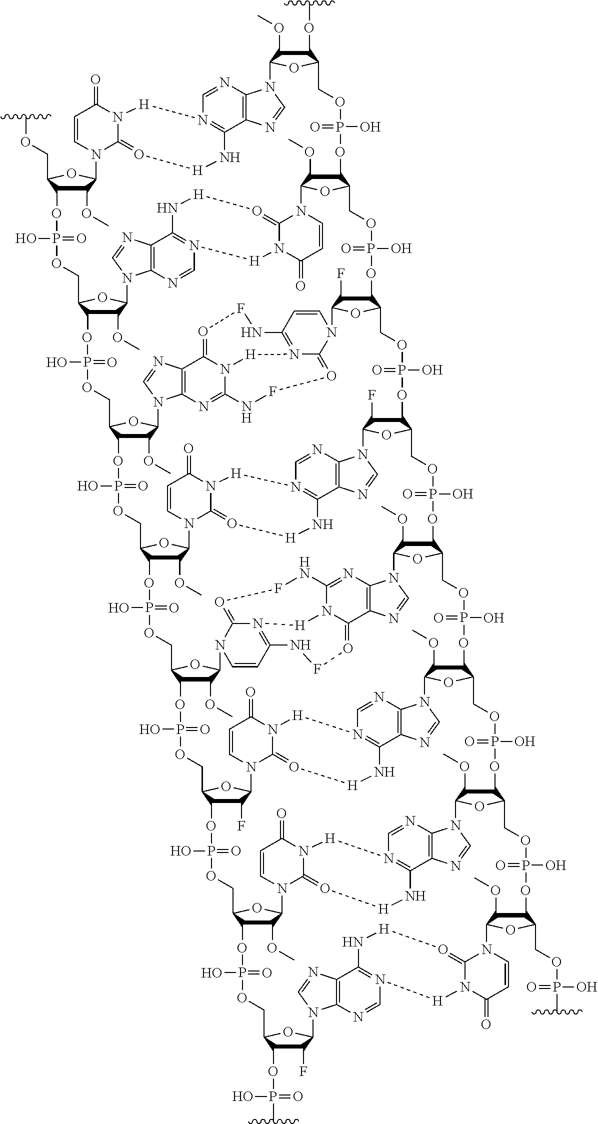

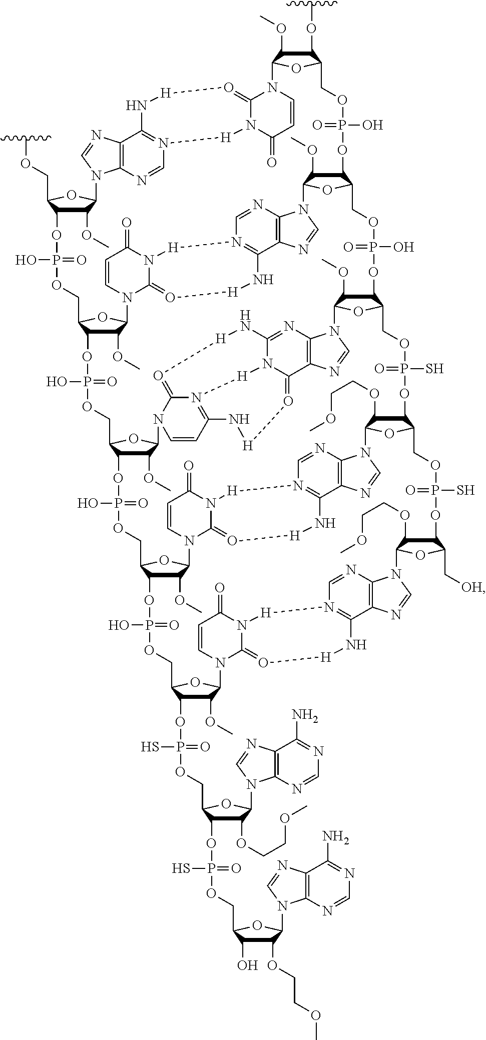

or a salt thereof.

Embodiment 272. The oligomeric duplex of embodiment 271, which is the sodium salt or potassium salt.

Embodiment 273. An oligomeric duplex according to the following chemical structure:

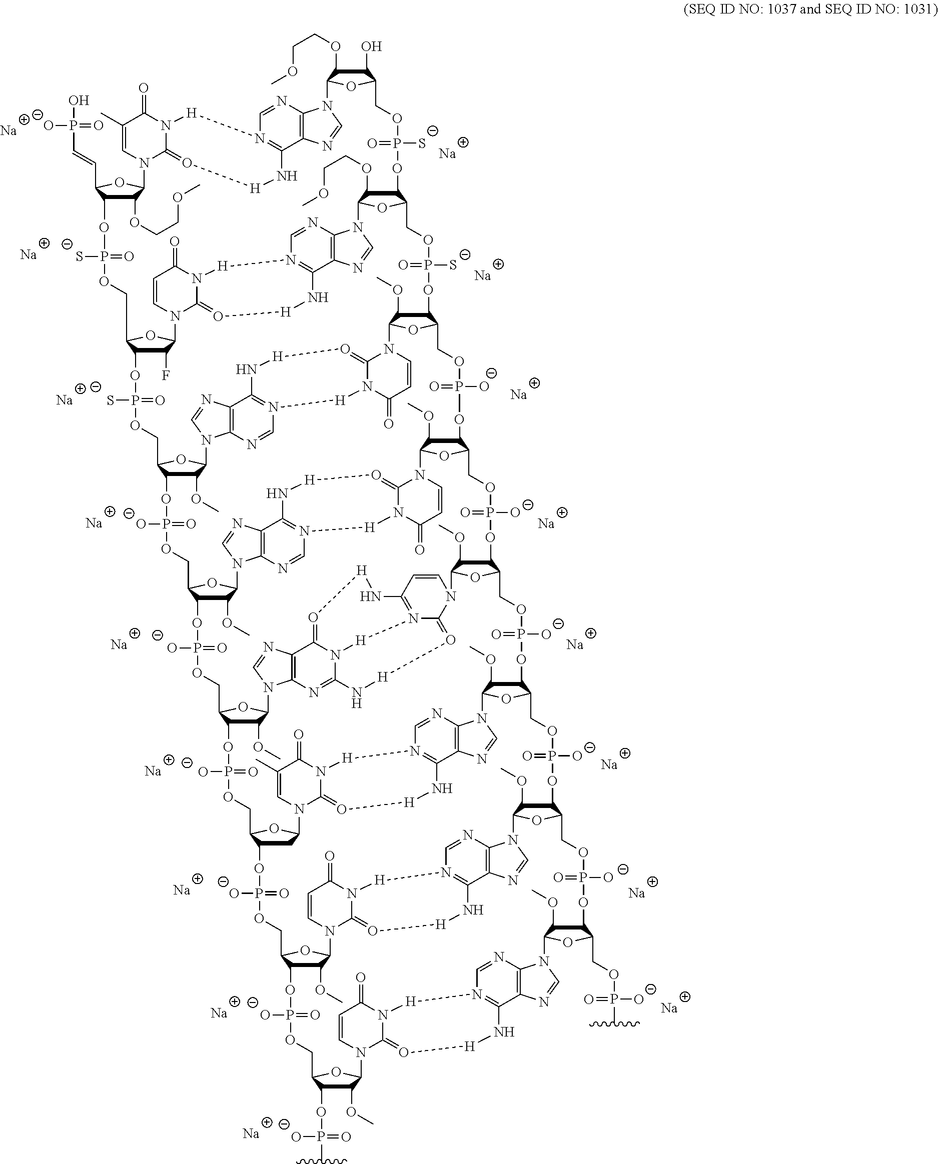

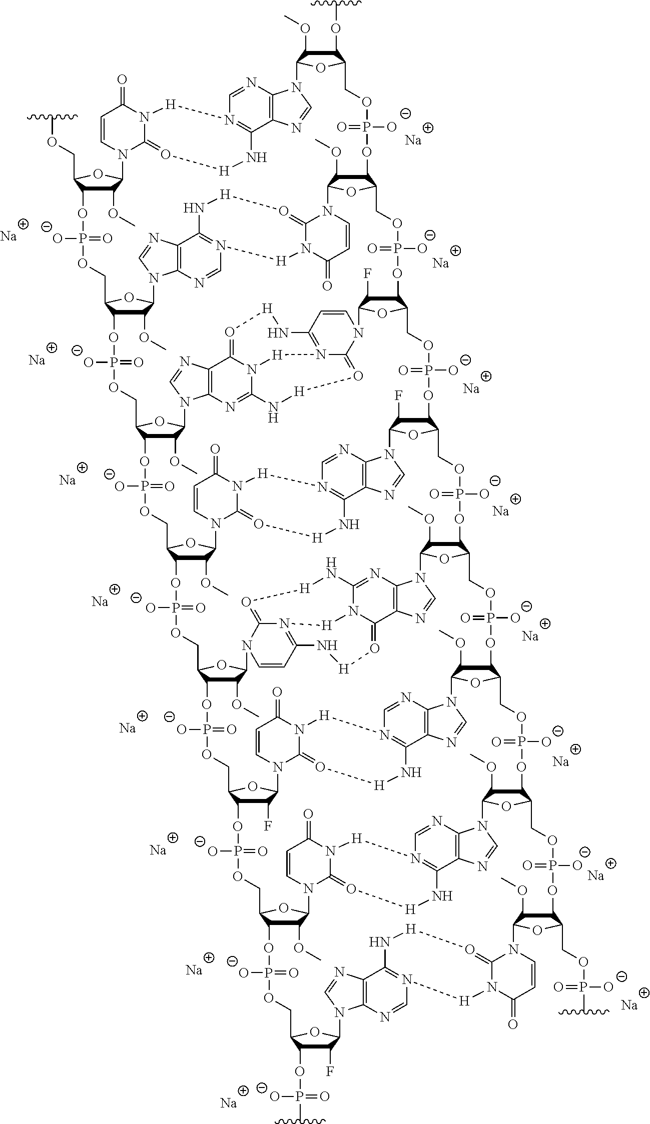

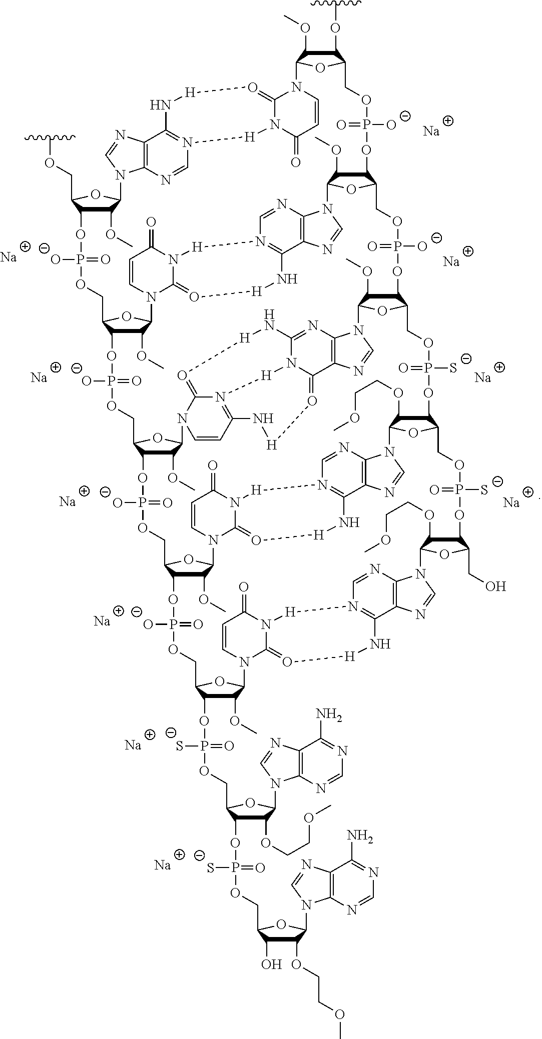

Embodiment 274. An oligomeric duplex according to the following chemical structure:

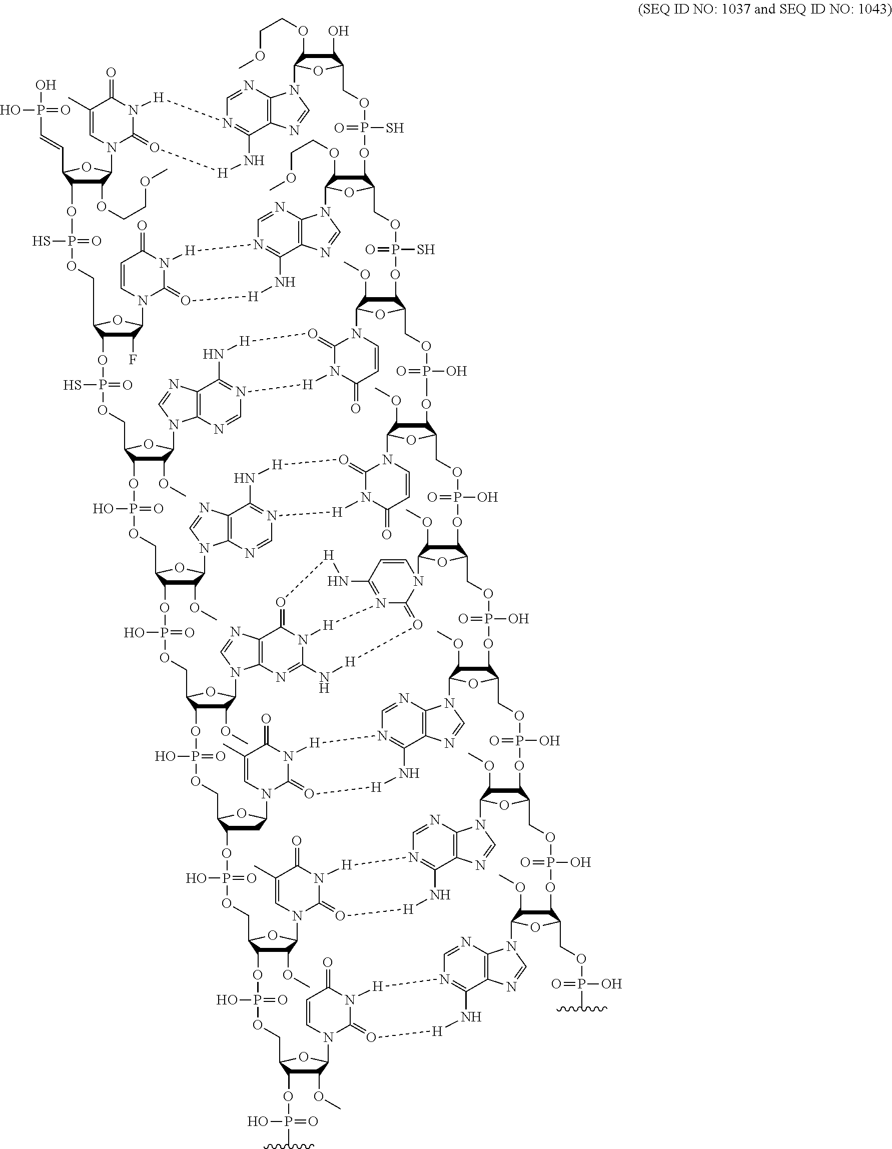

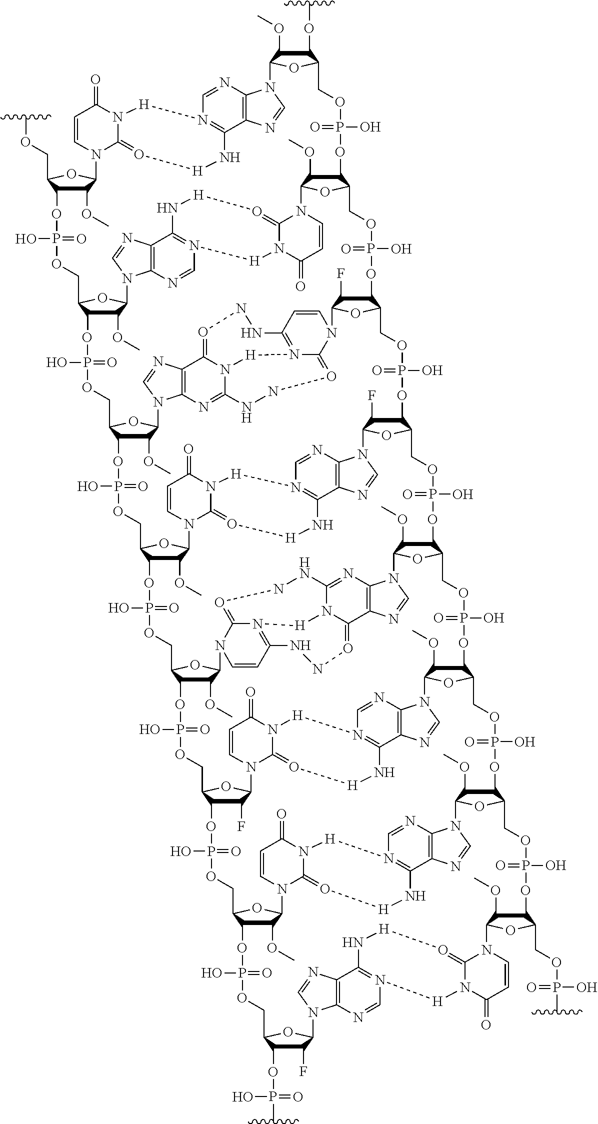

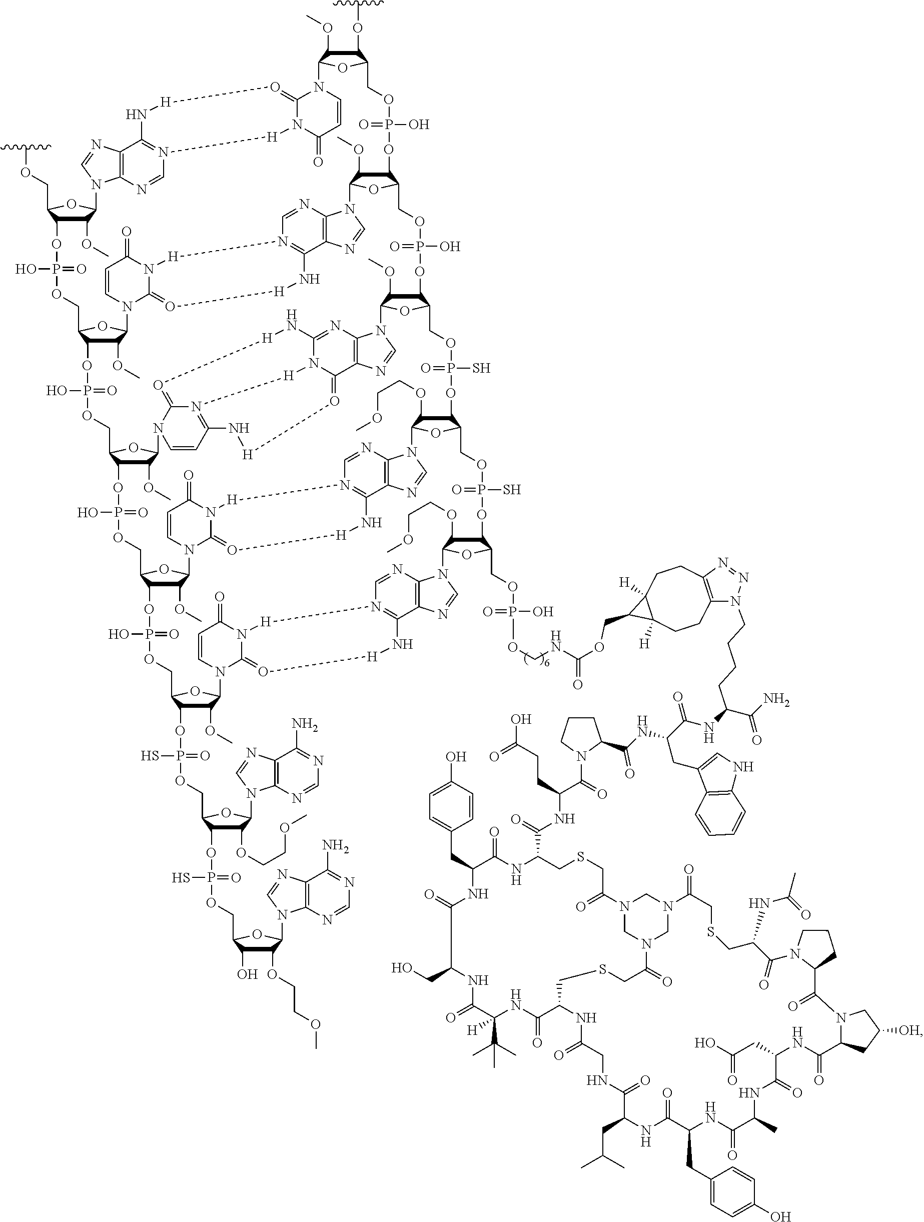

or a salt thereof.

Embodiment 275. The oligomeric duplex of embodiment 274, which is the sodium salt or potassium salt.

Embodiment 276. An oligomeric duplex according to the following chemical structure:

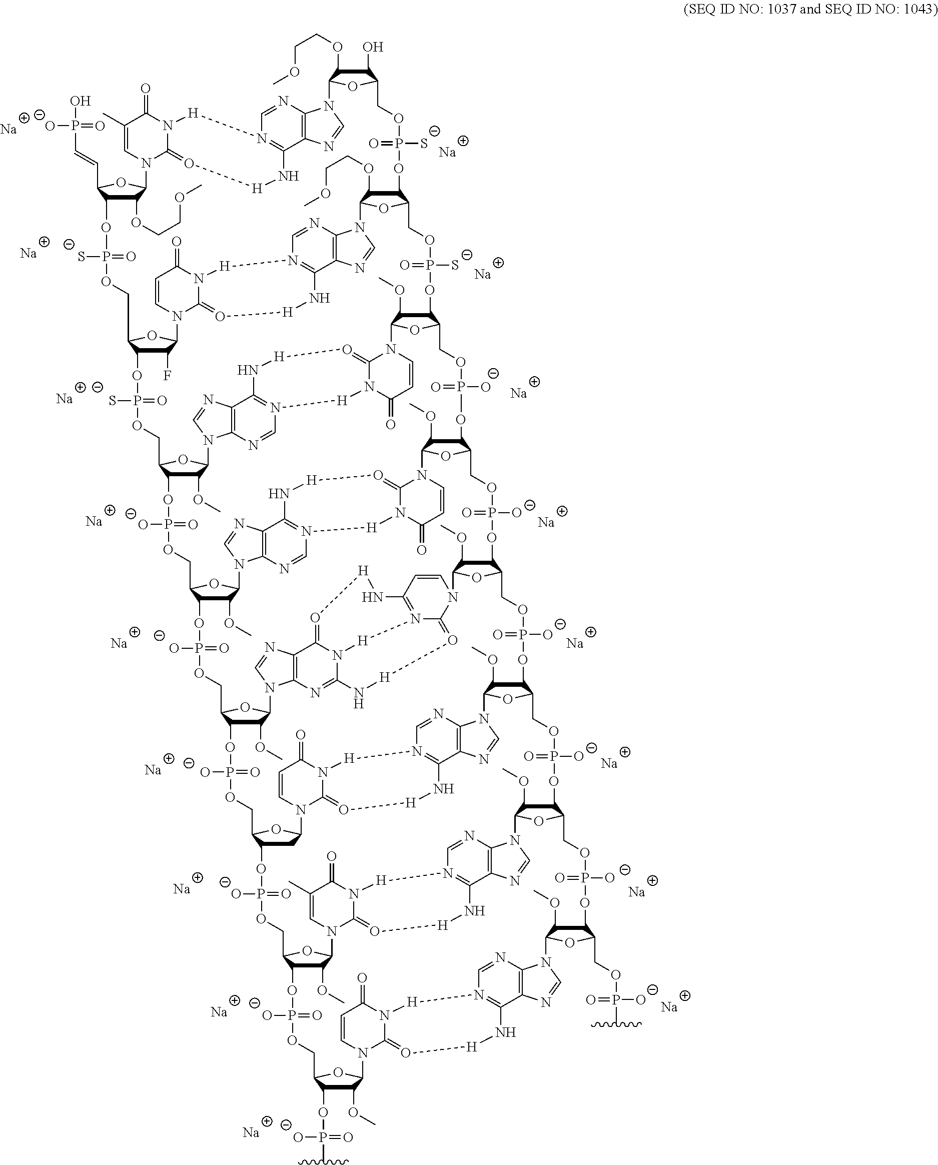

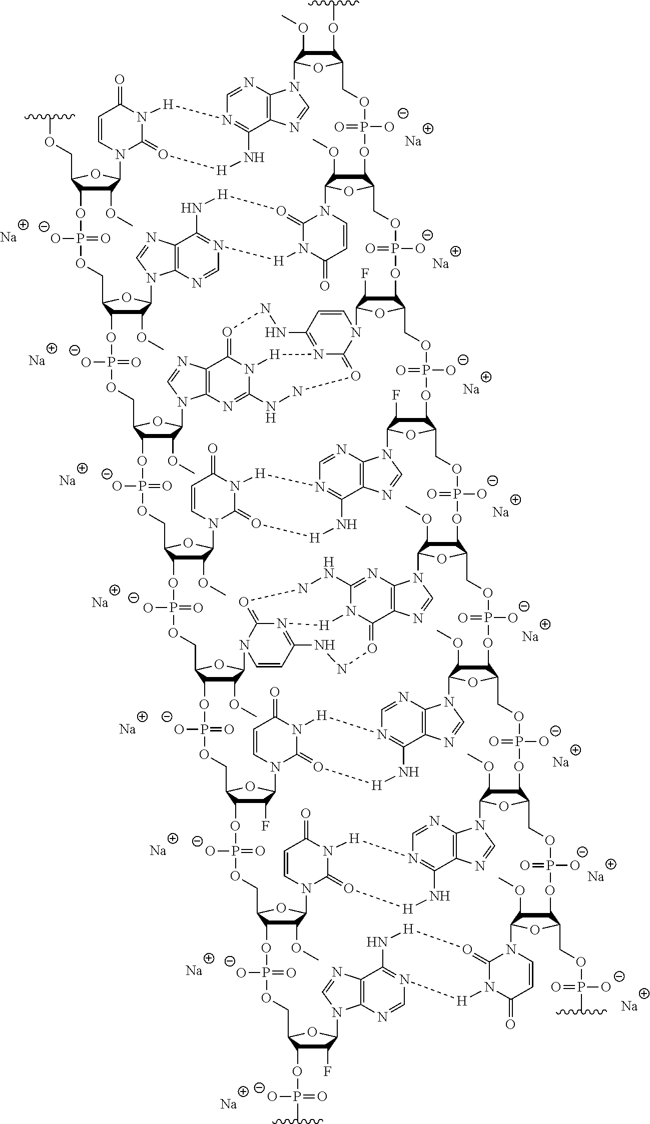

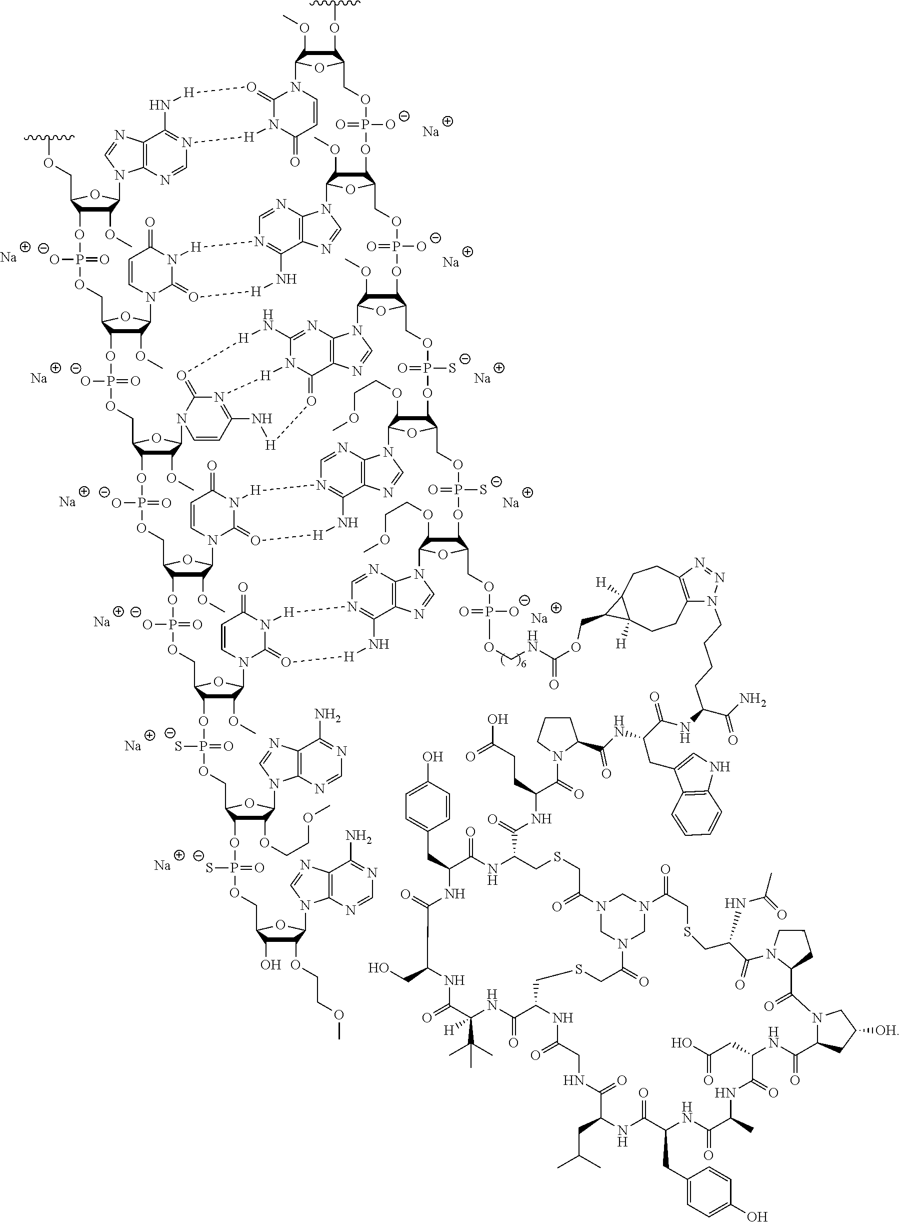

Embodiment 277. An oligomeric duplex according to the following chemical structure:

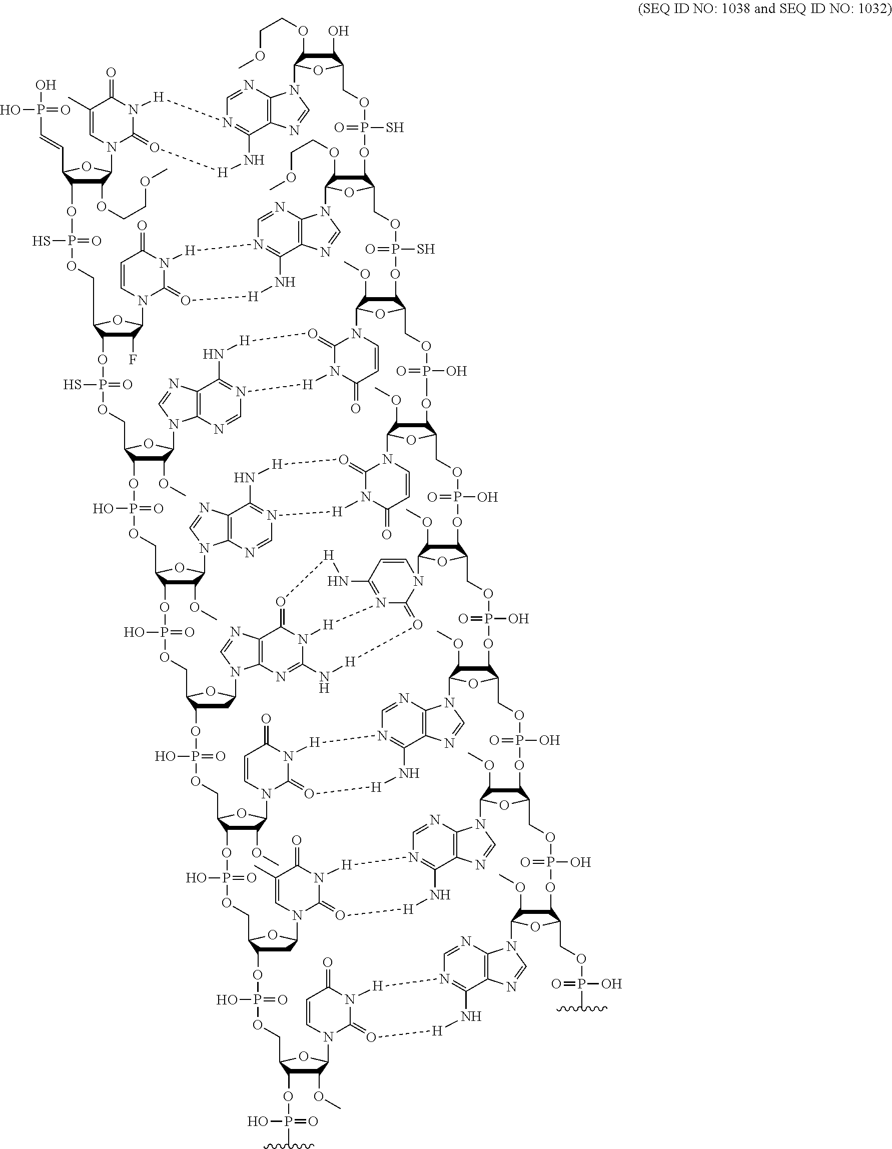

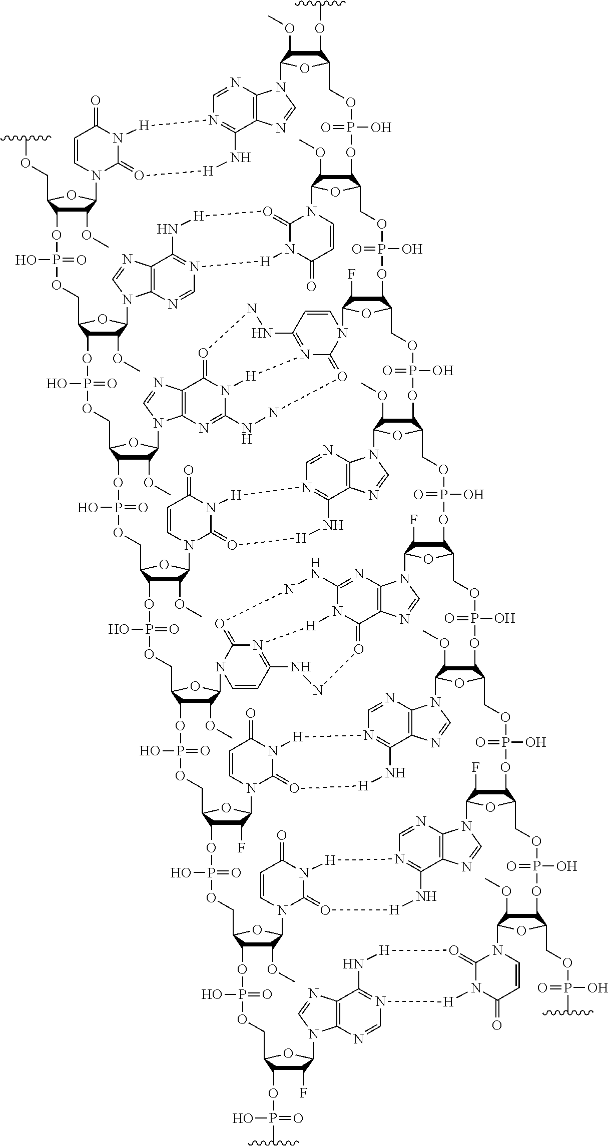

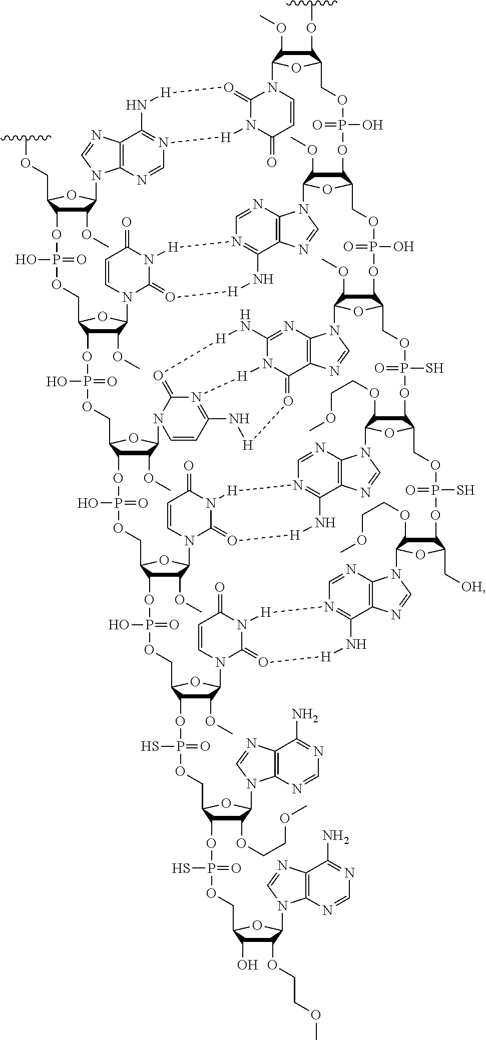

or a salt thereof.

Embodiment 278. The oligomeric duplex of embodiment 277, which is the sodium salt or potassium salt.

Embodiment 279. An oligomeric duplex according to the following chemical structure:

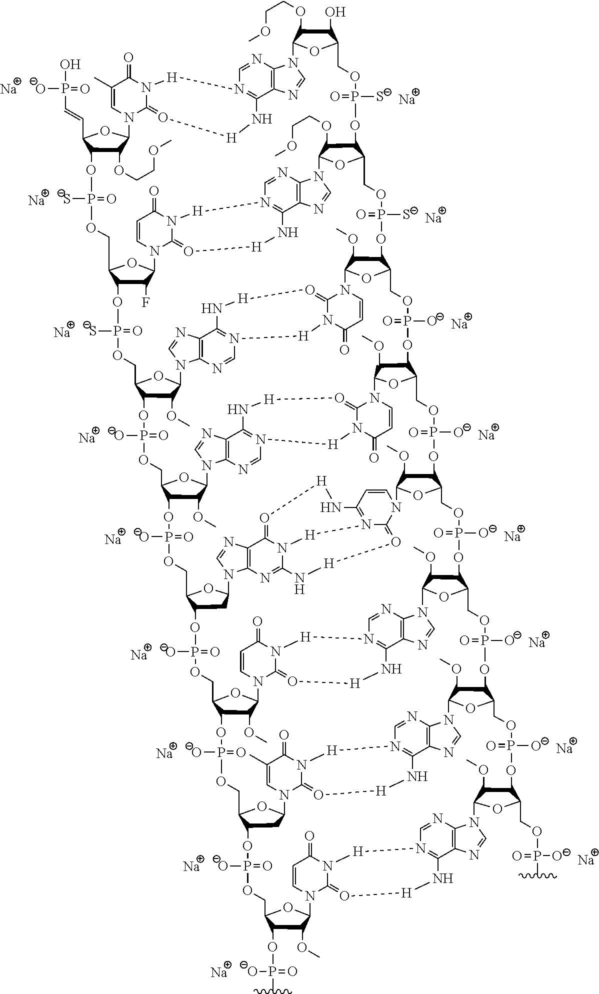

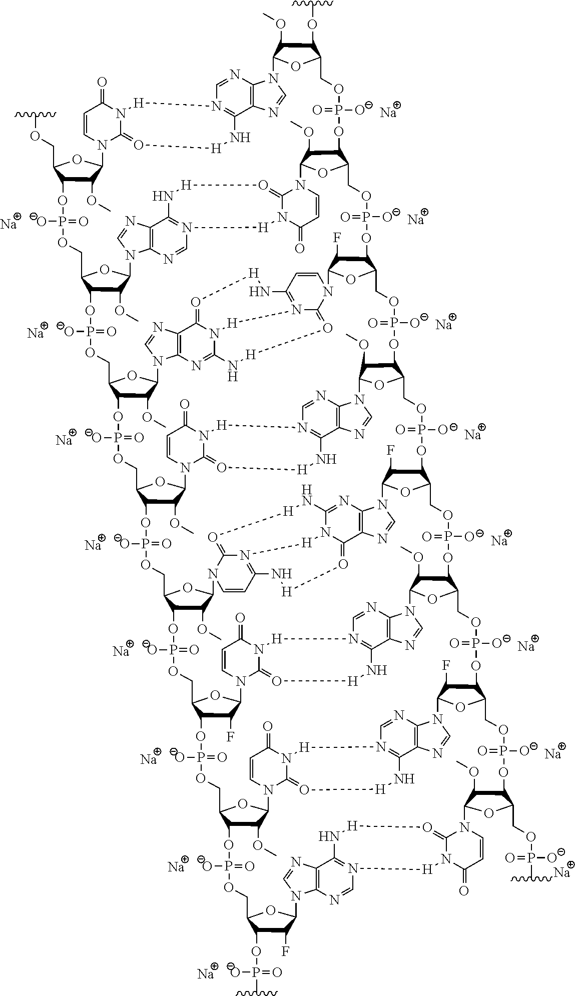

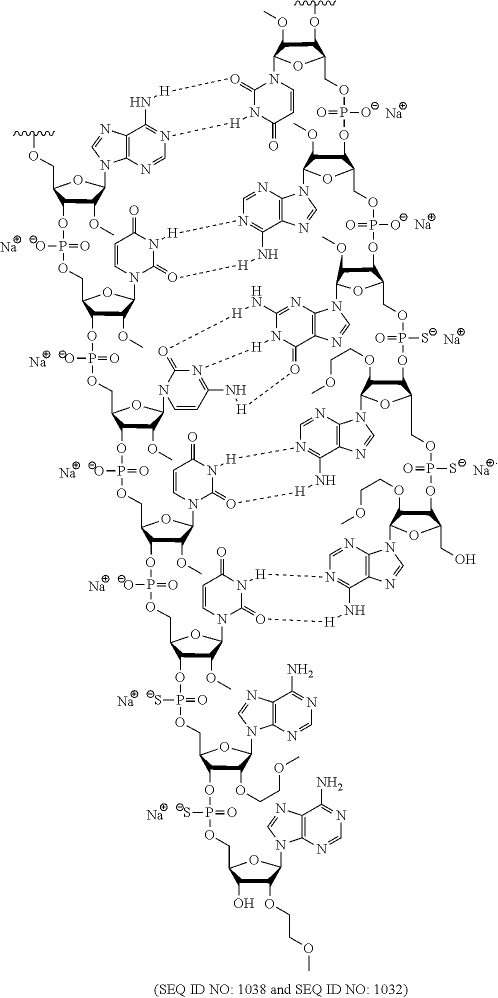

Embodiment 280. An oligomeric duplex according to the following chemical structure:

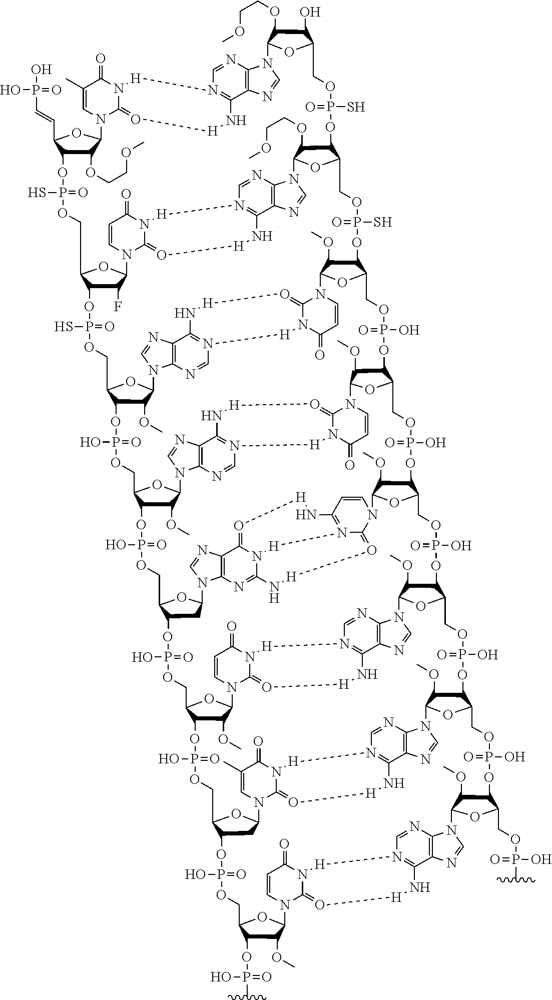

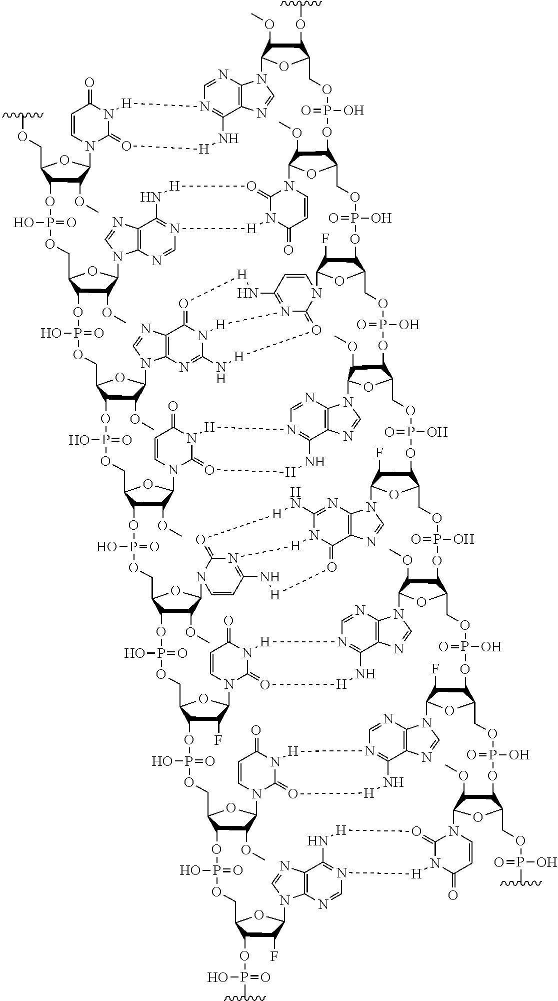

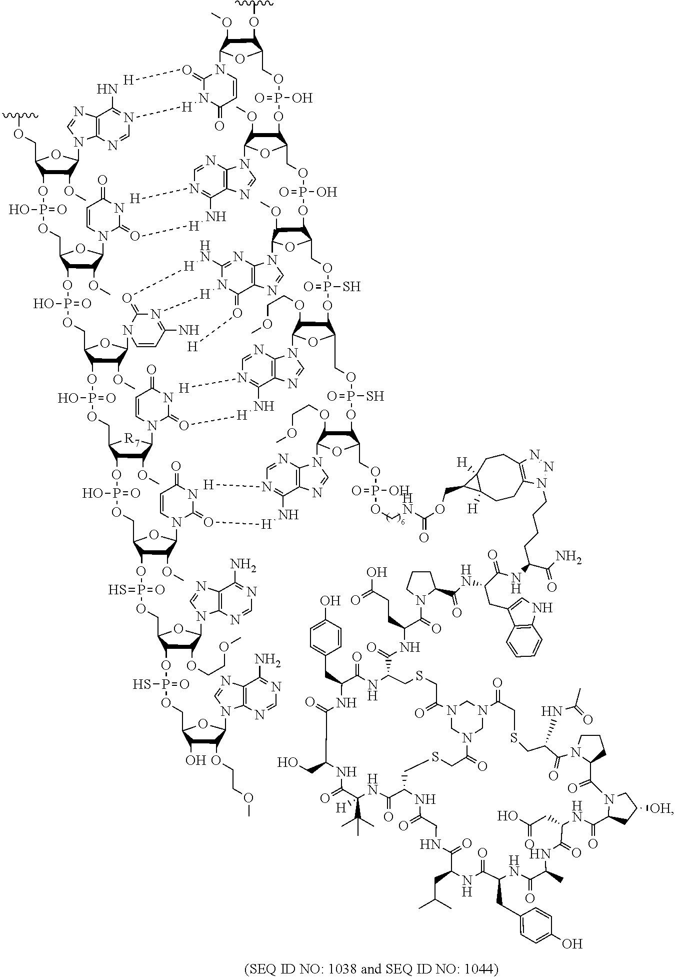

Embodiment 281. The oligomeric duplex of embodiment 280, which is the sodium salt or potassium salt.

Embodiment 282. An oligomeric duplex according to the following chemical structure:

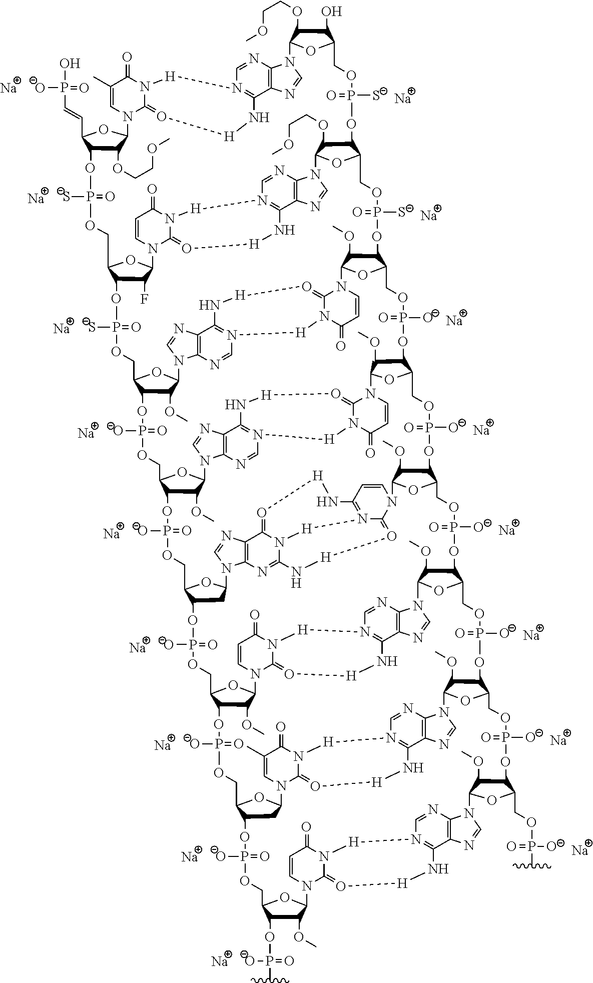

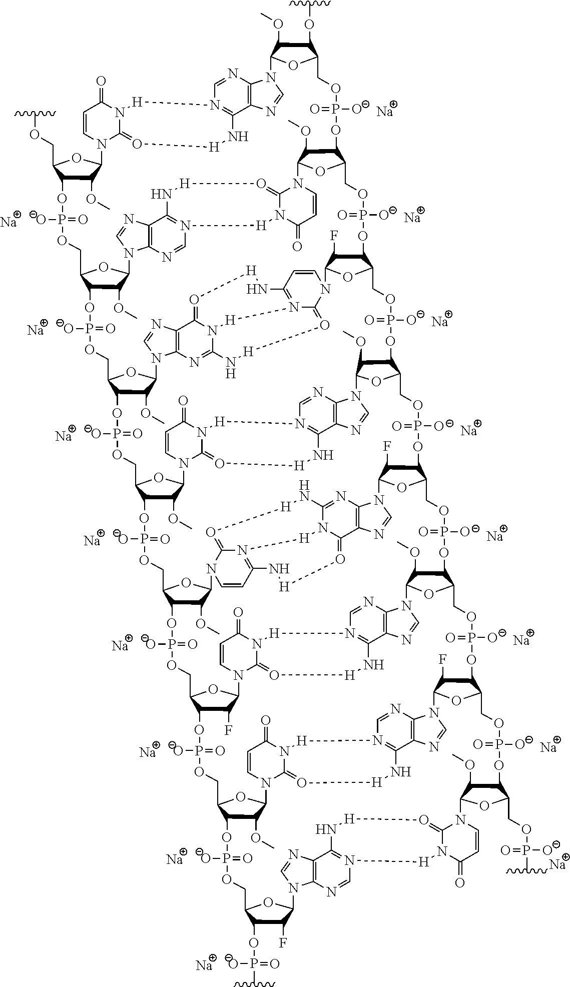

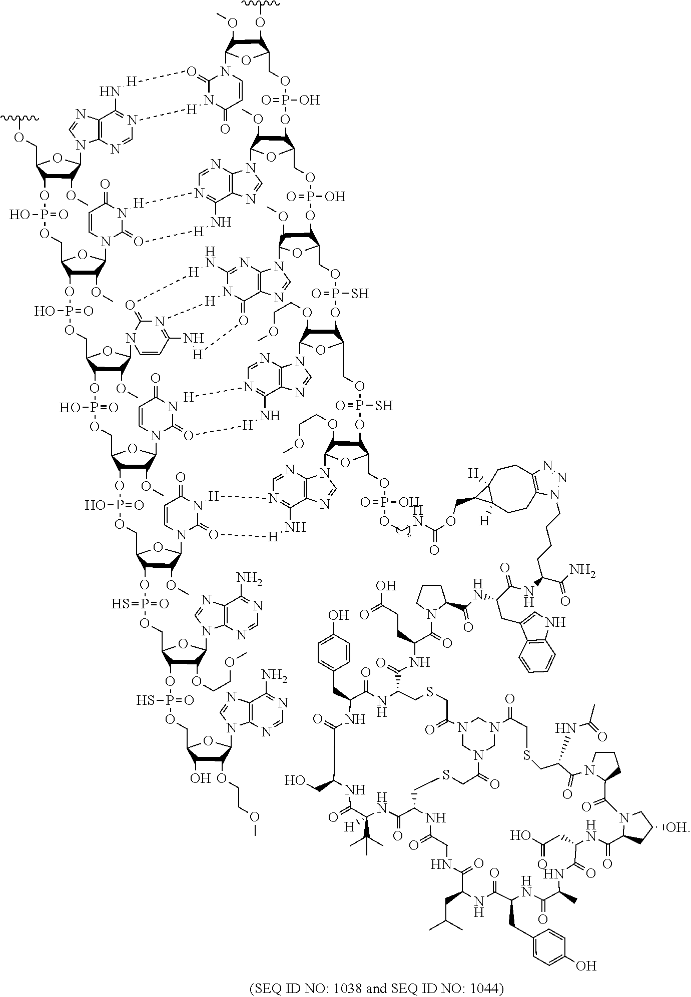

Embodiment 283. An oligomeric duplex according to the following chemical structure:

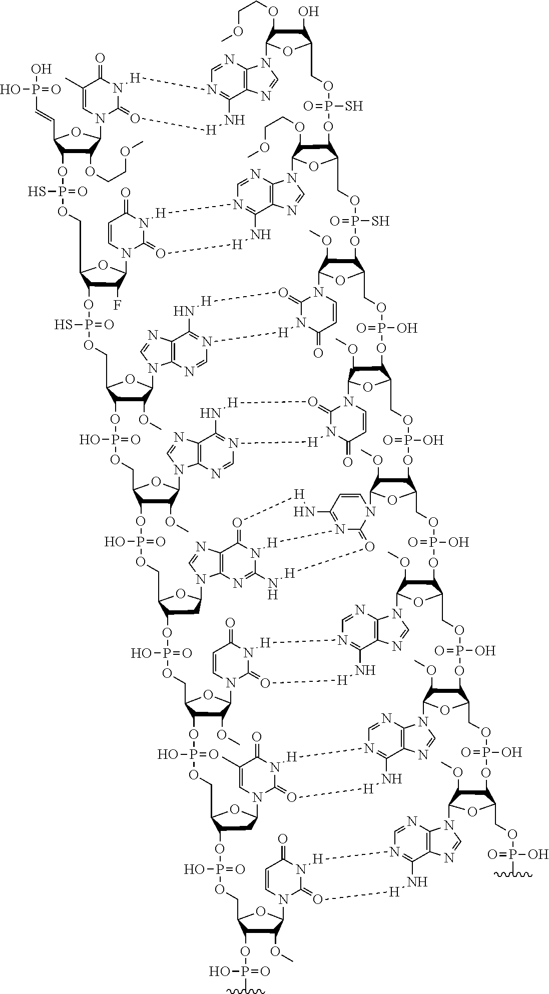

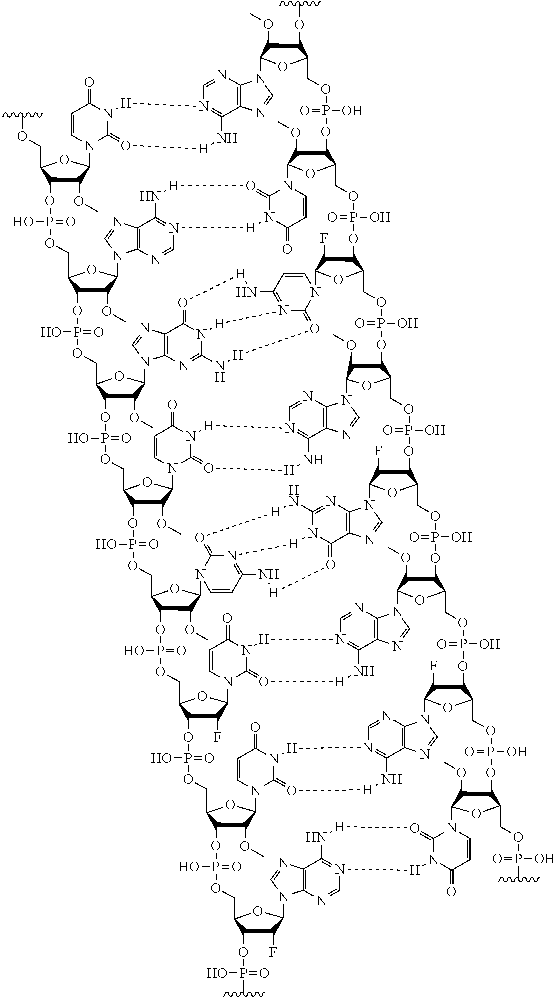

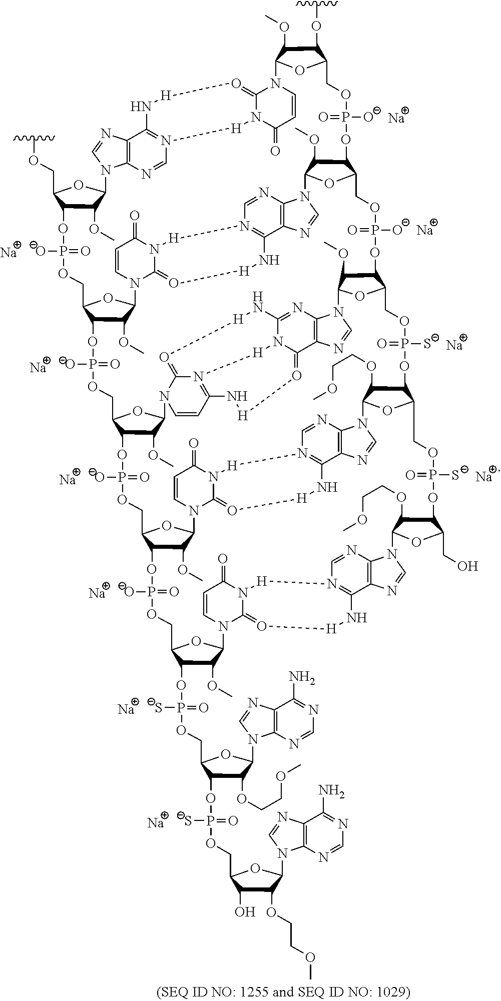

or a salt thereof.

Embodiment 284. The oligomeric duplex of embodiment 283, which is the sodium salt or potassium salt.

Embodiment 285. An oligomeric duplex according to the following chemical structure:

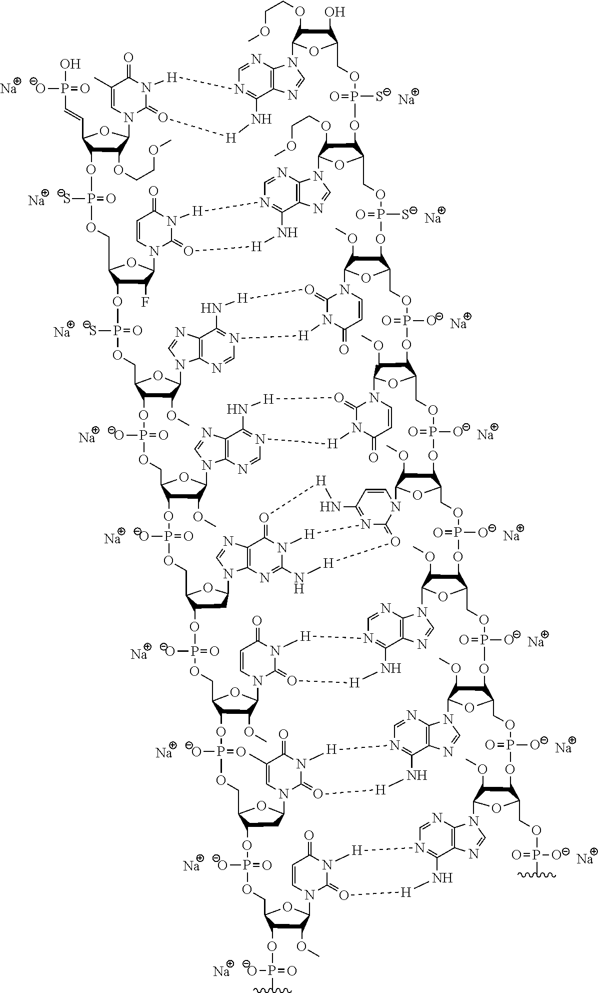

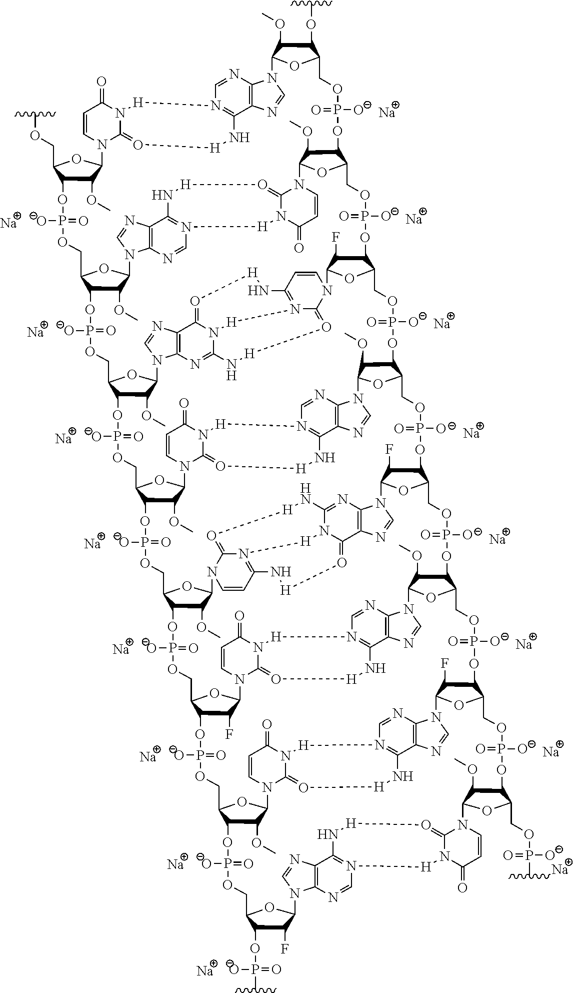

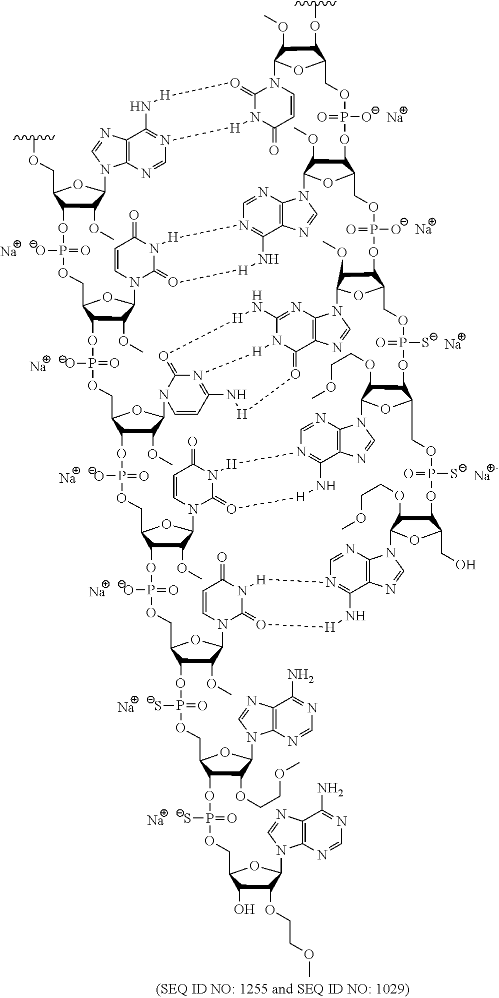

Embodiment 286. An oligomeric duplex according to the following chemical structure:

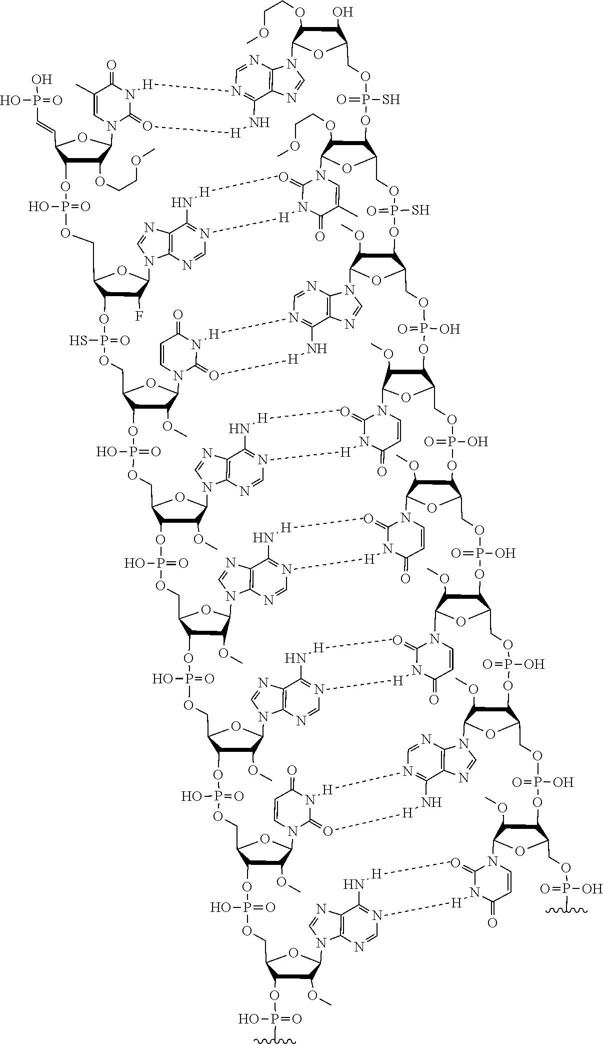

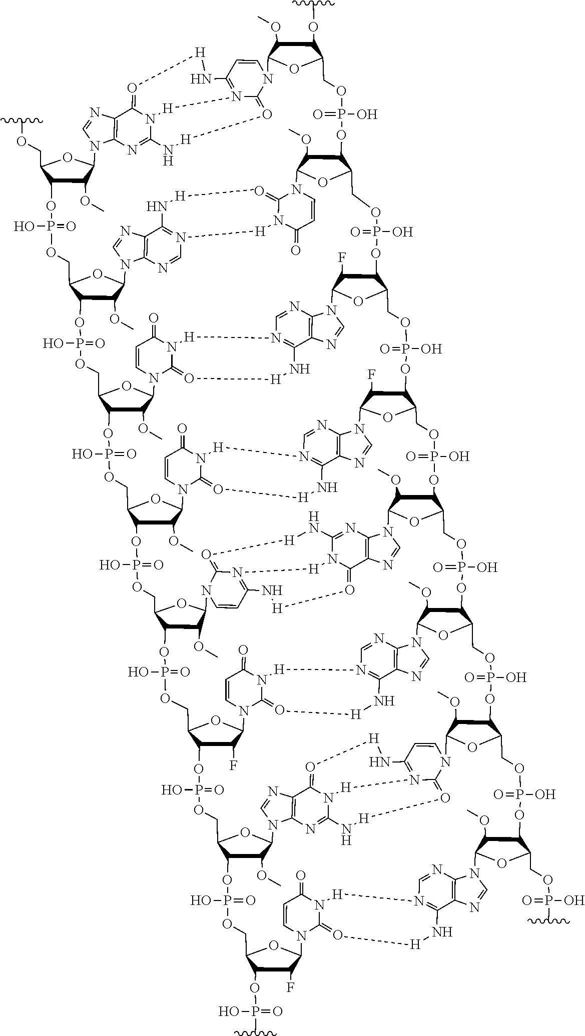

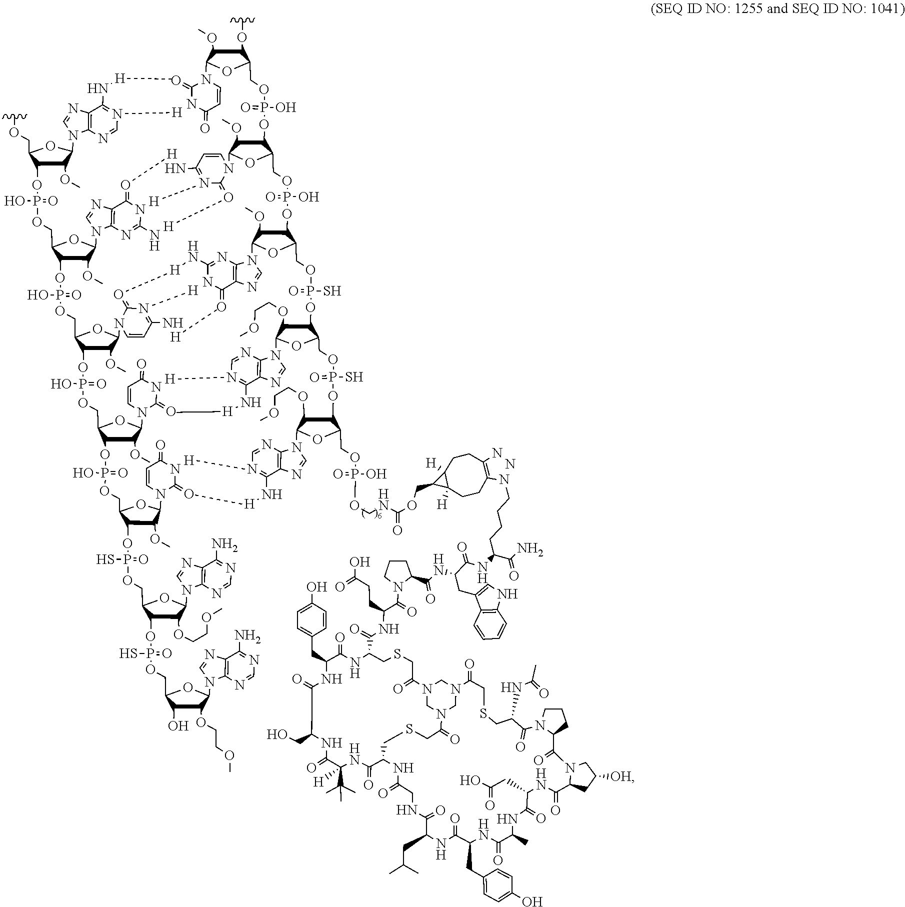

or a salt thereof.

Embodiment 287. The oligomeric duplex of embodiment 286, which is the sodium salt or potassium salt.

Embodiment 288. An oligomeric duplex according to the following chemical structure:

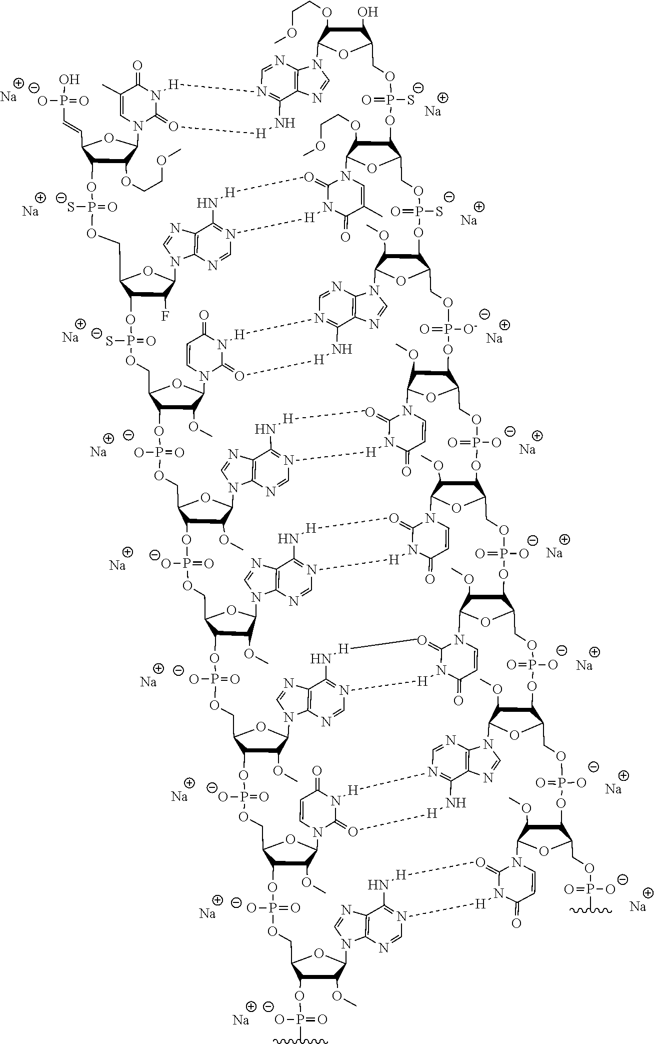

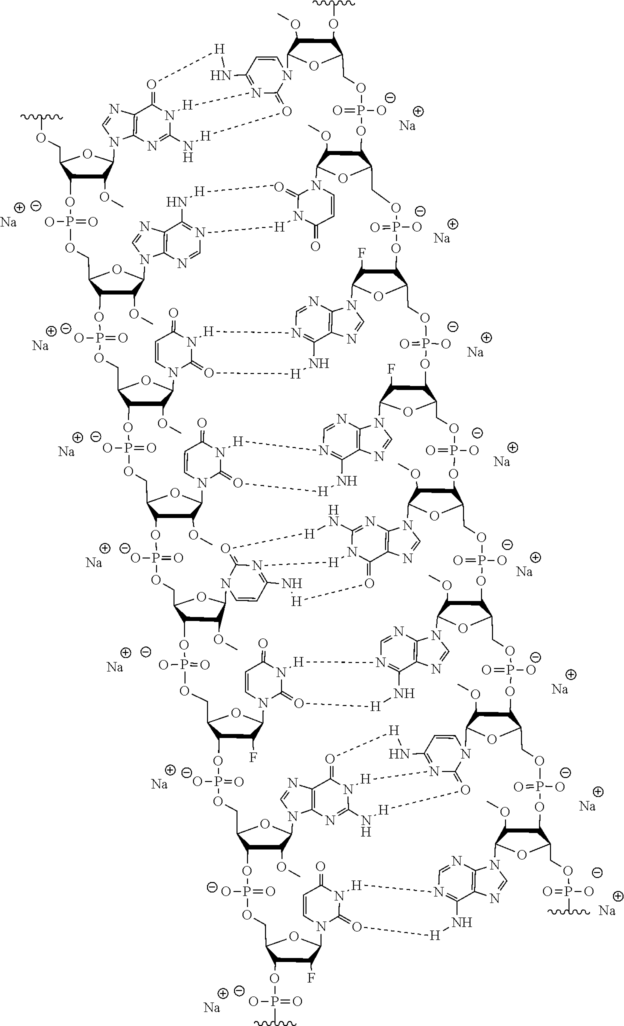

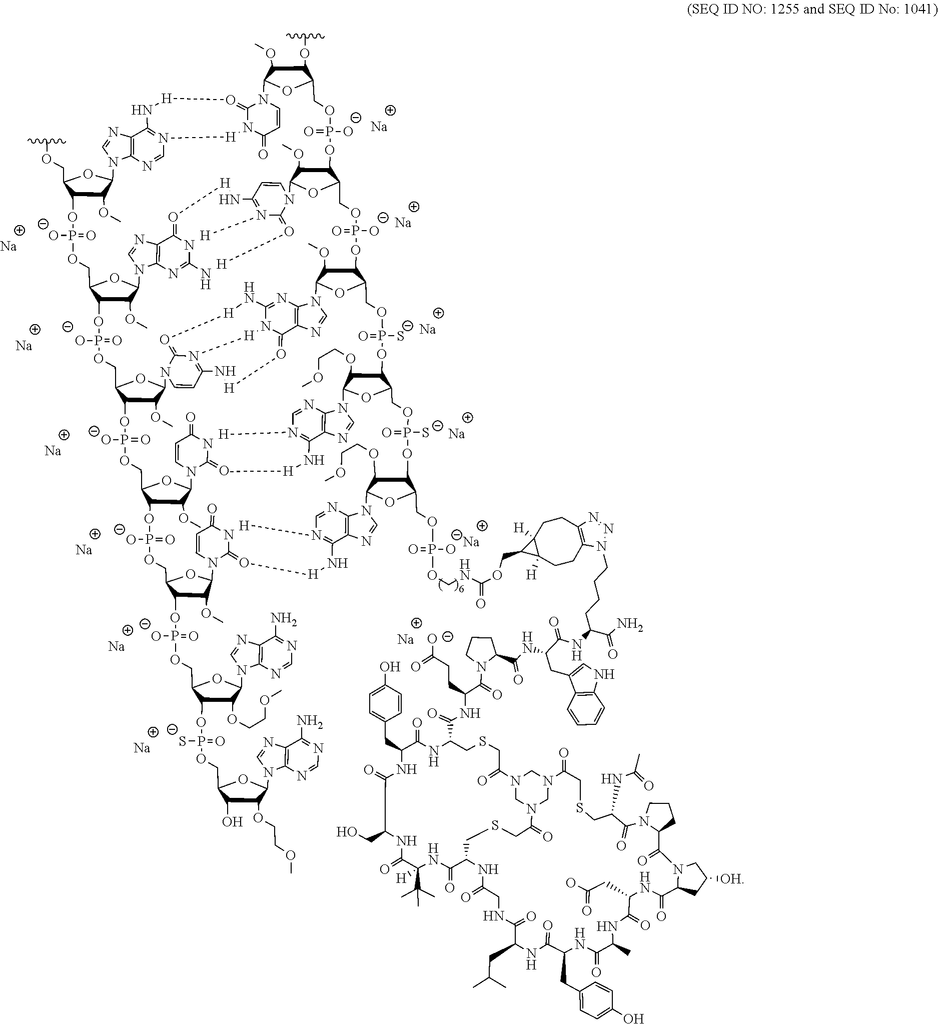

Embodiment 289. A population of oligomeric duplexes or oligomeric compounds of any one of embodiments 1-288, wherein the population is enriched for first and/or second modified oligonucleotides comprising at least one particular phosphorothioate internucleoside linkage having a particular stereochemical configuration.

Embodiment 290. The population of embodiment 289, wherein the population is enriched for first and/or second modified oligonucleotides comprising at least one particular phosphorothioate internucleoside linkage having the (Sp) or (Rp) configuration.

Embodiment 291. The oligomeric duplex or oligomeric compound of any one of embodiments 1-288, wherein the first modified oligonucleotide consists of 23 linked nucleosides and the second modified oligonucleotide consists of 21 linked nucleosides.

Embodiment 292. An antisense compound comprising or consisting of an oligomeric duplex or oligomeric compound of any one of embodiments 1-288.

Embodiment 293. The antisense compound of embodiment 292, wherein the antisense compound is an RNAi agent capable of reducing the amount of PLN nucleic acid through the activation of RISC/Ago2.

Embodiment 294. A pharmaceutical composition comprising the oligomeric duplex or oligomeric compound of any one of embodiments 1-288 or 291, the population of any one of embodiments 289-290, or the antisense compound of embodiment 292 or embodiment 293, and a pharmaceutically acceptable diluent or carrier.

Embodiment 295. The pharmaceutical composition of embodiment 294, wherein the pharmaceutically acceptable diluent is water or phosphate-buffered saline.

Embodiment 296. The pharmaceutical composition of embodiment 295, wherein the pharmaceutical composition consists essentially of the oligomeric duplex, oligomeric compound or the antisense compound, and water or phosphate-buffered saline.

Embodiment 297. A method of decreasing the amount of PLN RNA and/or PLN protein in a cell, tissue, organ or subject, comprising contacting the cell, tissue, organ or subject with the oligomeric duplex or oligomeric compound of any one of embodiments 1-288 or 291, the population of any one of embodiments 289-290, or the antisense compound of embodiment 292 or embodiment 293, or the pharmaceutical composition of any one of embodiments 294-296.

Embodiment 298. The method of embodiment 297, wherein the cell is a muscle cell and/or a cardiac cell.

Embodiment 299. A method comprising administering to a subject the oligomeric duplex or oligomeric compound of any one of embodiments 1-288 or 291, the population of any one of embodiments 289-290, or the antisense compound of embodiment 292 or embodiment 293, or the pharmaceutical composition of any one of embodiments 294-296; wherein the subject has or is at risk for developing a cardiovascular or cardiac injury, disease, condition or disorder, cardiomyopathy, cardiac arrythmia, and/or heart failure.

Embodiment 300. A method of preventing or treating a disease, disorder, condition or injury associated with cardiac calcium misregulation, or postponing a symptom of a disease, disorder or condition associated with a damaged, weakened and/or overworked heart, comprising administering to a subject having, or at risk of having, a disease, disorder or condition associated with cardiac calcium misregulation a therapeutically effective amount of the oligomeric duplex or oligomeric compound of any one of embodiments 1-288 or 291, the population of any one of embodiments 289-290, or the antisense compound of embodiment 292 or embodiment 293, or the pharmaceutical composition of any one of embodiments 294-296; wherein the disease, disorder, condition or injury is selected from a cardiac or cardiovascular disease, disorder, condition or injury, a cardiomyopathy, cardiac arrythmia, and/or heart failure.

Embodiment 301. A method of preventing or treating a cardiac or cardiovascular disease, disorder, condition or injury associated with heart failure, or postponing a symptom of heart failure, comprising administering to a subject having, or at risk of having, a cardiac or cardiovascular disease, disorder, condition or injury a therapeutically effective amount of the oligomeric duplex or oligomeric compound of any one of embodiments 1-288 or 291, the population of any one of embodiments 289-290, or the antisense compound of embodiment 292 or embodiment 293, or the pharmaceutical composition of any one of embodiments 294-296; wherein the disease, disorder, condition or injury is a cardiomyopathy, cardiac arrythmia, and/or heart failure.

Embodiment 302. A method of decreasing the amount of PLN RNA and/or PLN protein in the heart of a subject having or at risk of developing a disease, disorder or condition associated with a damaged, weakened and/or overworked heart, comprising administering to a subject having, or at risk of having, a disease, disorder or condition associated with a damaged, weakened and/or overworked heart a therapeutically effective amount of the oligomeric duplex or oligomeric compound of any one of embodiments 1-288 or 291, the population of any one of embodiments 289-290, or the antisense compound of embodiment 292 or embodiment 293, or the pharmaceutical composition of any one of embodiments 294-296; wherein the disease, disorder, condition or injury is a cardiomyopathy, cardiac arrythmia, and/or heart failure.

Embodiment 303. The method of any one of embodiments 297-302, wherein the amount of PLN RNA and/or PLN protein in cardiac muscle of the subject is decreased.

Embodiment 304. The method of any one of embodiments 297-302, wherein the disease, disorder or condition is cardiomyopathy or arrhythmia.

Embodiment 305. The method of embodiment 304, wherein the cardiomyopathy is genetic cardiomyopathy.

Embodiment 306. The method of embodiment 305, wherein the genetic cardiomyopathy is associated with PLN p.Arg14del, Arg9Cys (R9C), or Arg25Cys (R25C) genetic mutations.

Embodiment 307. The method of embodiment 304, wherein the cardiomyopathy is dilated cardiomyopathy (DCM).

Embodiment 308. The method of embodiment 307 wherein the DCM is genetic DCM.

Embodiment 309. The method of embodiment 308, wherein the genetic DCM is associated with TTN, LMNA, RBM20, SCN5A, MYH7, TNNT2, or TPM1 mutations.

Embodiment 310. The method of embodiment 307, wherein the DCM is arrhythmogenic DCM.

Embodiment 311. The method of any one of embodiments 299-303, wherein the heart failure is heart failure with preserved ejection fraction (HFpEF), heart failure with reduced ejection fraction (HFrEF), acute heart failure, or worsening of chronic heart failure.

Embodiment 312. The method of embodiment 304, wherein the arrhythmia is ventricular tachycardia (vtac) or ventricular fibrillation (vfib).

Embodiment 313. The method of embodiment 300 or embodiment 301, wherein the symptom of a disease, disorder or condition associated with a damaged, weakened and/or overworked heart, or heart failure is reduced myocardial contractile function and/or impaired relaxation of the heart.

Embodiment 314. The method of any one of embodiments 299-313, wherein the method prevents or slows progression of damaged, weakened and/or overworked heart effects and/or heart failure.

Embodiment 315. The method of any one of embodiments 299-313, wherein administering of the oligomeric duplex or oligomeric compound of any one of embodiments 1-288 or 291, the population of any one of embodiments 289-290, or the antisense compound of embodiment 292 or embodiment 293, or the pharmaceutical composition of any one of embodiments 294-296 improves cardiac function, cardiovascular death, cardiac dilation, cardiac fibrosis, low voltage ECG, diastolic calcium uptake, ejection fraction (EF), left ventricular ejection fraction (LVEF), left ventricular end systolic volume (LVESV), left ventricular end diastolic volume (LVEDV), mitral valve flow profile, left ventricle (LV) strain, left ventricle (LV) strain rate, infarct size, heart failure hospitalization, 6 minute walk test (6MWT), the Kansas City Cardiomyopathy Questionnaire Score (KCCQS), heart rate, or heart rhythm in the subject.

Embodiment 316. The method of any one of embodiments 297-315, wherein the oligomeric duplex or oligomeric compound of any one of embodiments 1-288 or 291, the population of any one of embodiments 289-290, or the antisense compound of embodiment 292 or embodiment 293, or the pharmaceutical composition of any one of embodiments 294-296 comprises a conjugate moiety having affinity for a transferrin receptor.

Embodiment 317. The method any one of embodiments 297-315, wherein the oligomeric duplex or oligomeric compound of any one of embodiments 1-288 or 291, the population of any one of embodiments 289-290, or the antisense compound of embodiment 292 or embodiment 293, or the pharmaceutical composition of any one of embodiments 294-296 comprises a bicycle ligand having affinity for a transferrin receptor.

Embodiment 318. The oligomeric duplex or oligomeric compound of any one of embodiments 1-288 or 291, the population of any one of embodiments 289-290, or the antisense compound of embodiment 292 or embodiment 293, or the pharmaceutical composition of any one of embodiments 294-296 for use in for treating or preventing a disease, disorder or condition associated with a damaged, weakened and/or overworked heart or heart failure.

Embodiment 319. Use of the oligomeric duplex or oligomeric compound of any one of embodiments 1-288 or 291, the population of any one of embodiments 289-290, or the antisense compound of embodiment 292 or embodiment 293, or the pharmaceutical composition of any one of embodiments 294-296 for treating or preventing a disease, disorder or condition associated with a damaged, weakened and/or overworked heart or heart failure.

Embodiment 320. Use of the oligomeric duplex or oligomeric compound of any one of embodiments 1-288 or 291, the population of any one of embodiments 289-290, or the antisense compound of embodiment 292 or embodiment 293, or the pharmaceutical composition of any one of embodiments 294-296 in the manufacture of a medicament for treating or preventing a disease, disorder or condition associated with a damaged, weakened and/or overworked heart or heart failure.

Embodiment 321. The use of any one of embodiments 318-320, wherein the disease, disorder or condition associated with a damaged, weakened and/or overworked heart or associated with heart failure is cardiomyopathy, cardiac arrythmia, and/or heart failure.

Embodiment 322. The use of embodiment 321, wherein the cardiomyopathy is genetic cardiomyopathy.

Embodiment 323. The use of embodiment 322, wherein the genetic cardiomyopathy is associated with PLN p.Arg14del, Arg9Cys (R9C), or Arg25Cys (R25C) genetic mutations.

Embodiment 324. The use of embodiment 321, wherein the cardiomyopathy is dilated cardiomyopathy (DCM).

Embodiment 325. The use of embodiment 324, wherein the DCM is genetic DCM.

Embodiment 326. The use of embodiment 325, wherein the genetic DCM is associated with TTN, LMNA, RBM20, SCN5A, MYH7, TNNT2, or TPM1 mutations.

Embodiment 327. The use of embodiment 324, wherein the DCM is arrhythmogenic DCM.

Embodiment 328. The use of embodiment 321, wherein the heart failure is heart failure with preserved ejection fraction (HFpEF), heart failure with reduced ejection fraction (HFrEF), acute heart failure, or worsening of chronic heart failure.

Embodiment 329. The use of embodiment 321, wherein the arrhythmia is ventricular tachycardia (vtac) or ventricular fibrillation (vfib).

Embodiment A1. An oligomeric duplex comprising a first oligomeric compound and a second oligomeric compound, wherein:
(1) a first oligomeric compound comprises a modified oligonucleotide consisting of 8 to 50 linked nucleosides, wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases of the nucleobase sequence of any of SEQ ID NOs: 3-314, 627-782, 939-976, 1033-1038, and 1254-1255 wherein each of the nucleosides of the first modified oligonucleotide comprises a modified sugar moiety or sugar surrogate and wherein at least one modified nucleoside and less than 40% of the nucleosides of the first modified oligonucleotide comprises a fluorine; and
(2) a second oligomeric compound comprises a modified oligonucleotide consisting of 8 to 50 contiguous linked nucleosides wherein the nucleobase sequence of the second modified oligonucleotide comprises at least 8, at least 9, at least 10, at least 11, at least 12, at least 13, at least 14, at least 15, at least 16, at least 17, at least 18, at least 19, at least 20, or at least 21 contiguous nucleobases of the nucleobase sequence of any of SEQ ID NOs: 315-626, 783-938, 977-1016 and 1027-1032, wherein each of the nucleosides of the second modified oligonucleotide comprises a modified sugar moiety or sugar surrogate and wherein less than 40% of the nucleosides of the second modified oligonucleotide comprises a fluorine. Embodiment A2. The oligomeric duplex of embodiment A1, wherein the first modified oligonucleotide is at least 80%, at least 85%, at least 90%, at least 95%, or at least 99% complementary to an equal length portion of a PLN nucleic acid, wherein the PLN nucleic acid has the nucleobase sequence of SEQ ID NO: 1 or SEQ ID NO: 2.

Embodiment A3. The oligomeric duplex of embodiment A1 or embodiment A2, wherein each of the nucleosides of the first modified oligonucleotide independently and the second modified oligonucleotide independently comprises a modified sugar moiety or sugar surrogate independently selected from 2′-F, 2′-MOE, 2′-OMe, 2′-deoxyribosyl, and FHNA.

Embodiment A4. The oligomeric duplex of any one of embodiments A1-A3, wherein fewer than 35%, fewer than 30%, fewer than 25%, fewer than 20%, fewer than 15%, fewer than 14%, fewer than 13%, fewer than 12%, fewer than 10%, fewer than 5%, or fewer than 1% of the nucleosides in the first modified oligonucleotide comprises a fluorine.

Embodiment A5. The oligomeric duplex of any one of embodiments A1-A4, wherein no more than 1 nucleoside, no more than 2 nucleosides, no more than 3 nucleosides, or no more than 4 nucleosides in the first modified oligonucleotide comprise a modified sugar moiety or sugar surrogate comprising a fluorine. Embodiment A6. The oligomeric duplex of any one of embodiments A1-A5, wherein no more than 1 nucleoside, no more than 2 nucleosides, or no more than 3 nucleosides within the sequence of the first modified oligonucleotide between and including the third and twenty-first nucleosides counting from the 5′ end of the modified oligonucleotide comprise a modified sugar moiety or sugar surrogate comprising a fluorine.

Embodiment A7. The oligomeric duplex of any one of embodiments A1-A6, wherein a nucleoside comprising a modified sugar moiety or sugar surrogate comprising a fluorine of the first modified oligonucleotide is independently selected from one of:
i. the second nucleoside counting from the 5′ end,
ii. the fourteenth and sixteenth nucleosides counting from the 5′ end, or
iii. the second, fourteenth and sixteenth nucleosides counting from the 5′ end; wherein each modified sugar moiety or sugar surrogate comprising a fluorine is independently a 2′-fluoro sugar moiety or a 3′-fluoro-hexitol sugar moiety.

Embodiment A8. The oligomeric duplex of any one of embodiments A1-A7, wherein no more than one of the modified sugar moiety and/or sugar surrogate comprising a fluorine in the first modified oligonucleotide is a 3′-fluoro-hexitol sugar moiety.

Embodiment A9. The oligomeric duplex of any one of embodiments A1-A8, wherein one or more nucleosides of the first modified oligonucleotide is a 2′-deoxynucleoside. Embodiment A10. The oligomeric duplex of embodiment A9, wherein the one or more 2′-deoxynucleosides is one or more nucleosides in a region of the sequence of the first modified oligonucleotide between and including the fifth nucleoside to the sixteenth nucleoside counting from the 5′ end of the first modified oligonucleotide.

Embodiment A11. The oligomeric duplex of embodiment A10, wherein the one or more 2′-deoxynucleosides is in a region of the sequence of the first modified oligonucleotide that is any of the fifth, sixth, and/or seventh nucleosides or that is any of the fourteenth, fifteenth, and/or sixteenth nucleosides counting from the 5′ end of the first modified oligonucleotide.

Embodiment A12. The oligomeric duplex of any one of embodiments A9-A11, wherein fewer than 20%, or fewer than 15%, of the nucleosides of the first modified oligonucleotide comprises a fluorine.

Embodiment A13. The oligomeric duplex of any one of embodiments A9-A12, wherein the one or more 2′-deoxynucleosides is the fifth, sixth and/or seventh nucleoside(s) counting from the 5′ end of the first modified oligonucleotide.

Embodiment A14. The oligomeric duplex of any one of embodiments A9-A13, wherein only two nucleosides of the first modified oligonucleotide are 2′-deoxynucleosides.

Embodiment A15. The oligomeric duplex of embodiment A14, wherein the two 2′-deoxynucleosides are the fifth and seventh nucleosides, or the fourteenth and sixteenth nucleosides, counting from the 5′ end of the first modified oligonucleotide.

Embodiment A16. The oligomeric duplex of embodiment A14, wherein the two 2′-deoxynucleosides are the fifth and seventh nucleosides counting from the 5′ end of the first modified oligonucleotide.

Embodiment A17. The oligomeric duplex of any one of embodiments A9-A13, wherein only one of the nucleosides of the first modified oligonucleotide is a 2′-deoxynucleoside.

Embodiment A18. The oligomeric duplex of embodiment A17, wherein the 2′-deoxynucleoside is the sixth or sixteenth nucleoside counting from the 5′ end of the first modified oligonucleotide.

Embodiment A19. The oligomeric duplex of embodiment A17, wherein the 2′-deoxynucleoside is the sixth nucleoside counting from the 5′ end of the first modified oligonucleotide.

Embodiment A20. The oligomeric duplex of any one of embodiments A1-A19, wherein one or more of the nucleosides of the first modified oligonucleotide independently comprises a sugar surrogate or a bicyclic sugar moiety.

Embodiment A21. The oligomeric duplex of embodiment A20, wherein the bicyclic sugar moiety comprises a 2′-4′ bridge selected from —O—CH₂—; and —O—CH(CH₃)—.

Embodiment A22. The oligomeric duplex of any one of embodiments A1-A21, wherein one or more of the nucleosides of the first modified oligonucleotide comprises a 2′-OMe sugar moiety.

Embodiment A23. The oligomeric duplex of embodiment A22, wherein at least 10%, at least 20%, at least 30%, at least 40%, at least 50% or at least 60% of the nucleosides of the first modified oligonucleotide comprise a 2′-OMe sugar moiety.

Embodiment A24. The oligomeric duplex of embodiment A22 or embodiment A23, wherein the one or more nucleosides comprising a 2′-OMe sugar moiety are in a region of the sequence of the first modified oligonucleotide between and including the third and twenty-first nucleosides counting from the 5′ end of the first modified oligonucleotide.

Embodiment A25. The oligomeric duplex of embodiment A22 or embodiment A23, wherein the 3′-terminal nucleoside of the first modified oligonucleotide comprises a 2′-OMe sugar moiety and/or the nucleoside immediately 5′ of the 3′-terminal nucleoside of the first modified oligonucleotide comprises a 2′-OMe sugar moiety.

Embodiment A26. The oligomeric duplex of any one of embodiments A1-A25, wherein one or more of the nucleosides of the first modified oligonucleotide comprise a 2′-MOE sugar moiety.

Embodiment A27. The oligomeric duplex of embodiment A26, wherein the 5′- and/or 3′-terminal nucleosides of the first modified oligonucleotide comprise a 2′-MOE sugar moiety.

Embodiment A28. The oligomeric duplex of any one of embodiments A1-A27, wherein the nucleoside immediately 5′ of the 3′-terminal nucleoside of the first modified oligonucleotide comprises a 2′-MOE sugar moiety.

Embodiment A29. The oligomeric duplex of any one of embodiments A1-A24 and A26-A28, wherein the 5′- and 3′-terminal nucleosides of the first modified oligonucleotide comprise a 2′-MOE sugar moiety and the nucleoside immediately 5′ of the 3′-terminal nucleoside of the first modified oligonucleotide comprises a 2′-MOE sugar moiety.

Embodiment A30. The oligomeric duplex of any one of embodiments A1-A29, wherein the first modified oligonucleotide comprises a stabilized phosphate group attached to the 5′-terminal nucleoside.

Embodiment A31. The oligomeric duplex of embodiment A30, wherein the stabilized phosphate group comprises a cyclopropyl phosphonate or a vinyl phosphonate.

Embodiment A32. The oligomeric duplex of any one of embodiments A1-A31, wherein the duplex comprises a conjugate group comprising a conjugate moiety and a conjugate linker.

Embodiment A33. The oligomeric duplex of embodiment A32, wherein the conjugate group comprises a cell-targeting moiety.

Embodiment A34. The oligomeric duplex of embodiment A32, wherein the conjugate moiety comprises an active drug substance, an aliphatic chain, a lipid, a peptide, a protein, a hydrocarbon, a polyamine, a polyamide, a polyether, a thioether, an aptamer, an antibody or antibody fragment, a vitamin, a fatty acid, a carbohydrate, an intercalator or a reporter molecule.

Embodiment A35. The oligomeric duplex of embodiment A32, wherein the conjugate group comprises a C22 alkyl, C20 alkyl, C17 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, or C5 alkyl, wherein the alkyl chain optionally has one or more unsaturated bonds.

Embodiment A36. The oligomeric duplex of embodiment A32, wherein the conjugate group comprises a 6-palmitamidohexyl moiety or a 2-(hydroxymethyl)-6-palmitamidohexyl moiety.

Embodiment A37. The oligomeric duplex of embodiment A32, wherein the duplex comprises a conjugate moiety that binds type 1 transferrin receptor (TfR1).

Embodiment A38. The oligomeric duplex of embodiment A37, wherein the conjugate moiety is selected from antibody or fragment thereof, a protein or peptide or an aptamer capable of binding TfR1.

Embodiment A39. The oligomeric duplex of embodiment A38, wherein the conjugate moiety is a cyclic protein or cyclic peptide.

Embodiment A40. The oligomeric duplex of embodiment A37, wherein the conjugate group consists of the bicycle ligand and a conjugate linker.

Embodiment A41. The oligomeric duplex of embodiment A40, wherein the bicycle compound comprises a polypeptide consisting of 13-22 linked amino acids or amino acid mimetics and a molecular scaffold, wherein each of a first, a second, and a third amino acid of the polypeptide comprises a reactive group, each of which separately forms a bond with the molecular scaffold, thereby forming two polypeptide loops attached to the molecular scaffold.

Embodiment A42. The oligomeric duplex of embodiment A41, wherein the peptide of the bicycle compound has the following chemical notation:

(SEQ ID NO: 1045)
Ac-CP[HyP]DAYLGC[tBuGly]SYCEPW[K(N₃)]-NH₂,

Ac=acetyl,
C=cysteine,
P=proline,
D=aspartate,
A=alanine,
Y=tyrosine,
L=leucine,
G=glycine,
S=serine,
E=glutamate,
W=tryptophan,
[HyP]=trans-4-hydroxy-L-proline,
[tBuGly]=t-butyl-glycine, and
[K(N₃)]=6-azido lysine;
and wherein each cysteine forms a covalent bond with the molecular scaffold 1,1′,1″-(1,3,5-triazinane-1,3,5-triyl)tris(2-bromoethanone) (TATB).

Embodiment A43. The oligomeric duplex of embodiment A41, wherein the bicycle compound has the structure:

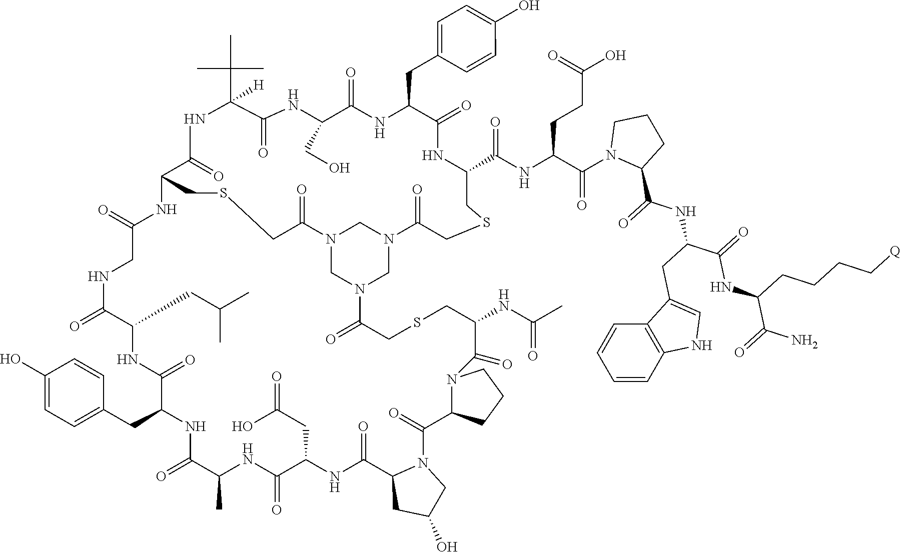

or a salt thereof, wherein Q is N₃ (BCY17901, SEQ ID NO: 1045), NH₂ (BCY21757, SEQ ID NO: 1202), SH (BCY21758, SEQ ID NO: 1203), a conjugate linker, or a conjugate linker covalently connected to an oligonucleotide.

Embodiment A44. The oligomeric duplex of any one of embodiments A32-A43, wherein the second oligomeric compound comprises the conjugate group conjugated to the second modified oligonucleotide.

Embodiment A45. The oligomeric duplex of any one of embodiments A32-A43, wherein the conjugate group is conjugated to the 5′ end or 3′ end of the second modified oligonucleotide.

Embodiment A46. The oligomeric duplex of any one of embodiments A32-A43, wherein the conjugate group is attached to the 5′-terminal nucleoside or the 3′-terminal nucleoside of the second modified oligonucleotide.

Embodiment A47. The oligomeric duplex of any one of embodiments A32-A43, wherein the conjugate group is attached to the 5′-terminal nucleoside of the second modified oligonucleotide.

Embodiment A48. The oligomeric duplex of any one of embodiments A32-A43, wherein the conjugate group is attached to the 3′-terminal nucleoside of the second modified oligonucleotide.

Embodiment A49. The oligomeric duplex of any one of embodiments A32-A48, wherein the conjugate linker of the conjugate group consists of a single bond.

Embodiment A50. The oligomeric duplex of any one of embodiments A32-A48, wherein the conjugate linker of the conjugate group is cleavable.

Embodiment A51. The oligomeric duplex of any one of embodiments A32-A50, wherein the conjugate linker comprises 1 to 3 linker-nucleosides.

Embodiment A52. The oligomeric duplex of any one of embodiments A32-A48, wherein the conjugate linker comprises a (bicyclo[6.1.0]nonyne)-formyl (BCN) moiety.

Embodiment A53. The oligomeric duplex of embodiment A52, wherein the conjugate linker comprises a 6-(BCN-carbamate)-2-(hydroxymethyl)hexyl phosphoryl moiety or a 6-(BCN-carbamate)hexyl phosphoryl moiety.

Embodiment A54. The oligomeric duplex of embodiment A53, wherein the conjugate group has the structure:

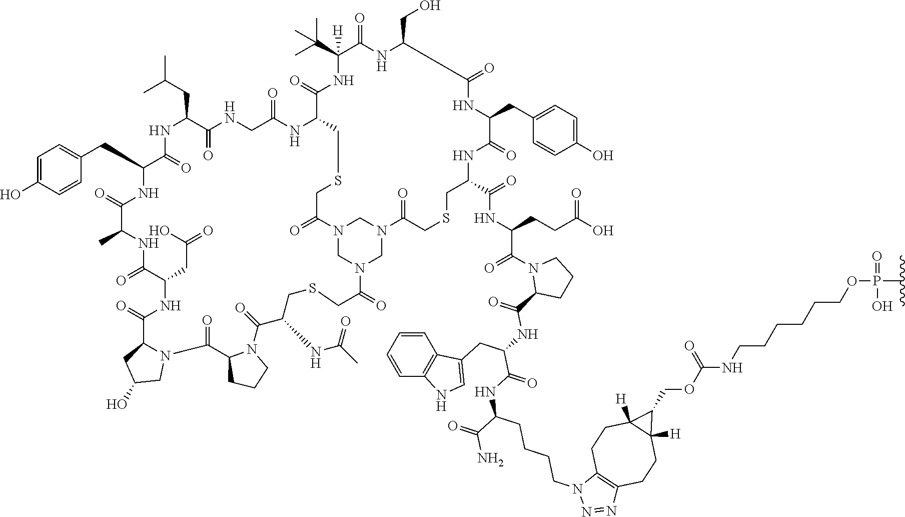

Embodiment A55. The oligomeric duplex of embodiment A53, wherein the conjugate group has the structure:

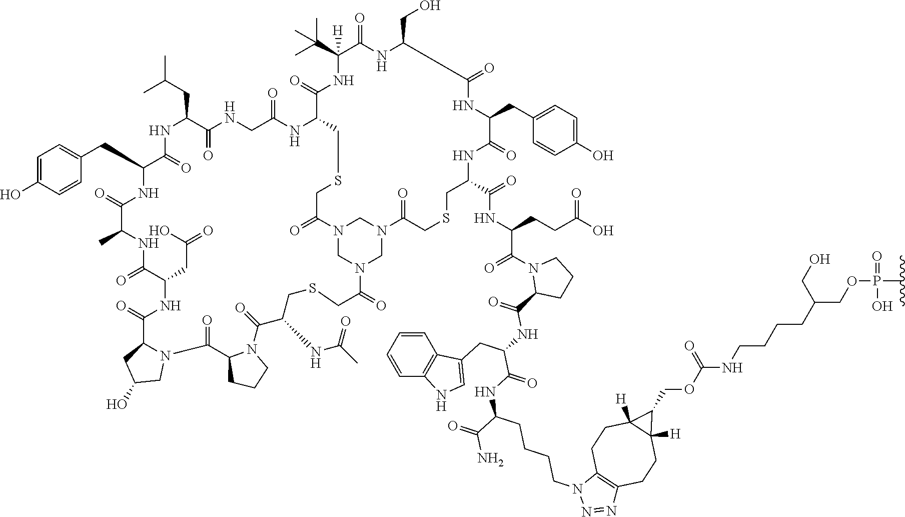

Embodiment A56. The oligomeric duplex of any one of embodiments A1-A55, wherein, the nucleobase sequence of the first modified oligonucleotide is at least 80%, at least 85%, at least 90%, at least 95%, or 100% complementary to an equal length portion within nucleobases 295-316, 296-317, 297-318, 298-320, 299-320, 300-321, 301-322, 302-324, 303-324, 304-325, 304-326, 305-327, 306-325, 306-328, 307-328, 308-329, 309-330, 310-331, 311-332, 312-333, 313-334, 314-335, 315-336, 316-337, 317-339, 318-339, 319-340, 320-341, 321-342, 322-344, 323-345, 324-345, 324-346, 325-345, 325-347, 326-347, 327-348, 328-349, 329-350, 330-351, 331-352, 332-353, 333-354, 334-355, 505-526, 506-528, 507-529, 508-530, 509-531, 510-531, 511-532, 512-533, 513-535, 514-535, 514-536, 515-536, 516-535, 516-537, 517-538, 518-539, 519-541, 520-541, 521-542, 522-544, 523-544, 524-546, 535-556, 536-557, 537-558, 538-559, 539-560, 540-561, 541-563, 542-563, 543-564, 544-565, 545-566, 546-567, 547-568, 548-570, 549-571, 550-572, 551-572, 552-574, 553-574, 554-575, 555-577, 556-578, 557-579, 558-580, 559-580, 560-581, 561-582, 562-583, 563-584, 595-616, 596-618, 597-618, 598-620, 599-621, 600-622, 601-623, 602-623, 603-624, 604-625, 605-627, 606-628, 607-628, 608-629, 609-630, 610-631, 611-632, 612-633, 613-635, 665-687, 666-687, 667-689, 668-689, 669-690, 670-691, 671-692, 672-694, 673-694, 674-695, 675-696, 676-697, 677-698, 678-700, 679-701, 680-702, 681-703, 682-703, 683-704, 684-705, 685-706, 686-708, 687-708, 688-709, 689-710, 690-711, 691-712, 692-714, 693-715, 1675-1696, 1676-1698, 1677-1698, 1678-1699, 1679-1700, 1680-1702, 1681-1702, 1682-1703, 1683-1705, 1684-1705, 1685-1706, 1686-1707, 1687-1709, 1688-1709, 1689-1710, 1690-1712, 1691-1713, 1692-1714, and 1693-1714 of SEQ ID NO: 1.

Embodiment A57. The oligomeric duplex of any one of embodiments A1-A55, wherein the nucleobase sequence of the first modified oligonucleotide is at least 80%, at least 85%, at least 90%, at least 95%, or 100% complementary to an equal length portion within nucleobases 304-326, 306-325, 324-346, 325-345, 514-536, and 516-535 of SEQ ID NO: 1.

Embodiment A58. The oligomeric duplex of any one of embodiments A1-A55, wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases of any of the nucleobase sequences of any of SEQ ID NOs: 3-314, 627-782, 939-976, 1033-1038, and 1254-1255.

Embodiment A59. The oligomeric duplex of any one of embodiments A1-A55, wherein the nucleobase sequence of the first modified oligonucleotide comprises the nucleobase sequence of any of SEQ ID NOs: 3-314, 627-782, 939-976, 1033-1038, and 1254-1255.

Embodiment A60. The oligomeric duplex of any one of embodiments A1-A55, wherein the nucleobase sequence of the first modified oligonucleotide consists of the nucleobase sequence of any of SEQ ID NOs: 3-314, 627-782, 939-976, 1033-1038, and 1254-1255.

Embodiment A61. The oligomeric duplex of any one of embodiments A1-A55, wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases of any of the nucleobase sequences of SEQ ID NOs: 29,171,176, 967, 968, 971, 972, 974, 975, 1254, 1255, and 1033-1038.

Embodiment A62. The oligomeric duplex of any one of embodiments A1-A55, wherein the nucleobase sequence of the first modified oligonucleotide comprises the nucleobase sequence of any of SEQ ID NOs: 29,171,176, 967, 968, 971, 972, 974, 975, 1254, 1255, and 1033-1038.

Embodiment A63. The oligomeric duplex of any one of embodiments A1-A55, wherein the nucleobase sequence of the first modified oligonucleotide consists of the nucleobase sequence of any one of SEQ ID NOs: 29,171,176, 967, 968, 971, 972, 974, 975, 1254, 1255, and 1033-1038.

Embodiment A64. The oligomeric duplex of any one of embodiments A1-A55, wherein the first modified oligonucleotide and the second modified oligonucleotide each independently consist of 18 to 30, 18 to 25, 18 to 24, 18 to 23, 18-22, 18-21, 18-19, 19 to 30, 19 to 25, 19 to 24, 19 to 23, 19-21, 19-20, 20 to 30, 20 to 25, 20 to 24, 20 to 23, 20 to 22, 20 to 21 linked nucleosides.

Embodiment A65. The oligomeric duplex of any one of embodiments A1-A55, wherein the first modified oligonucleotide consists of 23 nucleosides.

Embodiment A66. The oligomeric duplex of any one of embodiments A1-A65, wherein the first modified oligonucleotide has a sugar motif (5′ to 3′) selected from: efyyydyyyyyyyfyfyyyyyee, efyyydyyyyyyyfyfyyyyyyy, e[FHNA]yyydyyyyyyyfyfyyyyyee, e[FHNA]yyydyyyyyyyfyfyyyyyyy, efyydydyyyyyyfyfyyyyyee, efyydydyyyyyyfyfyyyyyyy, e[FHNA]yydydyyyyyyfyfyyyyyee, e[FHNA]yydydyyyyyyfyfyyyyyyy, efyyyfyyyyyyyfyfyyyyyyy, efyyyfyyyyyyyfyfyyyyyey, efyyyyyyyyyyyfyfyyyyyee, efyyyfyyyyyyyfyfyyyyyee, efyyyyyyyyyyyfyyyyyyyee, efyyyyyyyyyyyfyyyyyyyyy, e[FHNA]yyyfyyyyyyyfyfyyyyyee, e[FHNA]yyyfyyyyyyyfyfyyyyyyy, efyyydyyyyyyydydyyyyyee, efyyydyyyyyyydydyyyyyyy, efyydydyyyyyyfyfyyyyyyy, efyyyfyyyyyyyfyyyyyyyee, efyyyfyyyyyyyfyyyyyyyyy, efyyyyyyyyyyyfyfyyyyyee, efyyyyyyyyyyyfyfyyyyyyy, e[FHNA]yydydyyyyfyfyfyyyyyyy, efyyyfyyyyyyyfydyyyyyyy, efyyyfyyyyyyyfydyyyyyee, efyyyfyyyyyyyfyfyyyee, and efyyyfyyyyyyyfyfyyyyy, wherein each “d” represents a 2′-β-D-deoxyribosyl sugar moiety, each “y” represents a 2′-OMe sugar moiety, each “e” represents a 2′-MOE sugar moiety, each “f” represents a 2′-fluoro sugar moiety, and each “[FHNA]” represents a 3′-fluoro-hexitol sugar moiety.

Embodiment A67. The oligomeric duplex of any one of embodiments A1-A65, wherein the first modified oligonucleotide has a sugar motif (5′ to 3′) selected from: efyydydyyyyyyfyfyyyyyee, efyyydyyyyyyyfyfyyyyyee, efyydydyyyyyyfyfyyyyyyy, e[FHNA]yydydyyyyfyfyfyyyyyyy, and efyyydyyyyyyyfyfyyyyyyy.

Embodiment A68. The oligomeric duplex of any one of embodiments A1-A67, wherein the first modified oligonucleotide comprises at least one modified internucleoside linkage.

Embodiment A69. The oligomeric duplex of embodiment A68, wherein at least one modified internucleoside linkage is a phosphorothioate or mesyl phosphoramidate internucleoside linkage.

Embodiment A70. The oligomeric duplex of embodiment A68, wherein fewer than 50%, fewer than 40%, fewer than 30%, fewer than 20%, or fewer than 10% of the internucleoside linkages of the first modified oligonucleotide are modified internucleoside linkages.

Embodiment A71. The oligomeric duplex of any of embodiments A1-A70, wherein each internucleoside linkage of the first modified oligonucleotide is independently selected from a phosphodiester internucleoside linkage and a phosphorothioate internucleoside linkage.

Embodiment A72. The oligomeric duplex of embodiment A71, wherein the internucleoside linkages between the first and second nucleosides and the second and third nucleosides counting from the 5′ end of the first modified oligonucleotide are modified internucleoside linkages.

Embodiment A73. The oligomeric duplex of embodiment A71, wherein the internucleoside linkages between the first and second nucleosides and the second and third nucleosides counting from the 3′ end of the first modified oligonucleotide are modified internucleoside linkages.

Embodiment A74. The oligomeric duplex of embodiment A72, wherein the internucleoside linkages between the first and second nucleosides and the second and third nucleosides counting from the 5′ end of the first modified oligonucleotide, and the internucleoside linkages between the first and second nucleosides and the second and third nucleosides counting from the 3′ end of the first modified oligonucleotide are modified internucleoside linkages.

Embodiment A75. The oligomeric duplex of any one of embodiments A71-A74, wherein the modified internucleoside linkages are phosphorothioate internucleoside linkages.

Embodiment A76. The oligomeric duplex of embodiment A74 or embodiment A75, wherein all other internucleoside linkages in the first modified oligonucleotide are phosphodiester internucleoside linkages.

Embodiment A77. The oligomeric duplex of embodiment A76, wherein the first modified oligonucleotide has an internucleoside linkage motif of ssooooooooooooooooooss and a sugar motif (5′ to 3′) selected from: efyydydyyyyyyfyfyyyyyee, efyyydyyyyyyyfyfyyyyyee, efyydydyyyyyyfyfyyyyyyy, e[FHNA]yydydyyyyfyfyfyyyyyyy, and efyyydyyyyyyyfyfyyyyyyy; wherein each “d” represents a 2′-D-D-deoxyribosyl sugar moiety, each “y” represents a 2′-OMe sugar moiety, each “e” represents a 2′-MOE sugar moiety, each “f” represents a 2′-fluoro sugar moiety, each “[FHNA]” represents a 3′-fluoro-hexitol sugar moiety, each “o” represents a phosphodiester internucleoside linkage and each “s” represents a phosphorothioate internucleoside linkage.

Embodiment A78. The oligomeric duplex of any one of embodiments A1-A77, wherein fewer than 30%, fewer than 25%, fewer than 20%, fewer than 15%, fewer than 14%, fewer than 13%, fewer than 12%, fewer than 10%, or fewer than 5% of the nucleosides in the second modified oligonucleotide comprise a fluorine.

Embodiment A79. The oligomeric duplex of any one of embodiments A1-A77, wherein no more than 4 nucleosides, no more than 3 nucleosides, no more than 2 nucleosides, or no more than 1 nucleoside in the second modified oligonucleotide comprise a modified sugar moiety comprising a fluorine.

Embodiment A80. The oligomeric duplex of any one of embodiments A1-A77, wherein none of the nucleosides before the seventh or after the eleventh nucleoside counting from the 5′ end of the second modified oligonucleotide comprise a modified sugar moiety comprising a fluorine.

Embodiment A81. The oligomeric duplex of any one of embodiments A1-A77, wherein one or more of the seventh, ninth and eleventh nucleosides counting from the 5′ end of the second modified oligonucleotide comprises a modified sugar moiety comprising a fluorine.

Embodiment A82. The oligomeric duplex embodiment A81, wherein two or more of the seventh, ninth and eleventh nucleosides counting from the 5′ end of the second modified oligonucleotide comprise a modified sugar moiety comprising a fluorine.

Embodiment A83. The oligomeric duplex embodiment A81, wherein the seventh, ninth and eleventh nucleosides counting from the 5′ end of the second modified oligonucleotide comprise a modified sugar moiety comprising a fluorine.

Embodiment A84. The oligomeric duplex of any one of embodiments A1-A80, wherein one or both of the tenth and eleventh nucleosides counting from the 5′ end of the second modified oligonucleotide comprises a modified sugar moiety comprising a fluorine.

Embodiment A85. The oligomeric duplex of any one of embodiments A77-A84, wherein the modified sugar moiety comprising a fluorine is a 2′-fluoro sugar moiety.

Embodiment A86. The oligomeric duplex of any one of embodiments A1-A85, wherein fewer than 30%, fewer than 25%, fewer than 20%, fewer than 15%, fewer than 14%, fewer than 13%, fewer than 12%, fewer than 10%, fewer than 5%, or fewer than 1% of the total nucleosides in the compound comprise a fluorine.

Embodiment A87. The oligomeric duplex of any one of embodiments A1-86, wherein one or more of the nucleosides of the second modified oligonucleotide independently comprises a sugar surrogate or a bicyclic sugar moiety.

Embodiment A88. The oligomeric duplex of embodiment A87, wherein the bicyclic sugar moiety comprises a 2′-4′ bridge selected from —O—CH₂—; and —O—CH(CH₃)—.

Embodiment A89. The oligomeric duplex of any one of embodiments A1-88, wherein one or more of the nucleosides of the second modified oligonucleotide comprises a 2′-OMe sugar moiety.

Embodiment A90. The oligomeric duplex of embodiment A89, wherein at least 10%, at least 20%, at least 30%, at least 40%, at least 50%, or at least 60%, or at least 65%, or at least 70%, or at least 80%, or at least 85%, or at least 90% of the nucleosides of the second modified oligonucleotide comprise a 2′-OMe sugar moiety.

Embodiment A91. The oligomeric duplex of embodiment A89 or embodiment A90, wherein the one or more nucleosides comprising a 2′-OMe sugar moiety are in a region of the sequence of the second modified oligonucleotide between and including the third and nineteenth nucleosides counting from the 5′ end of the second modified oligonucleotide.

Embodiment A92. The oligomeric duplex of embodiment A89 or embodiment A90, wherein the 3′-terminal nucleoside of the second modified oligonucleotide comprises a 2′-OMe sugar moiety and/or the nucleoside immediately 5′ of the 3′-terminal nucleoside of the second modified oligonucleotide comprises a 2′-OMe sugar moiety.

Embodiment A93. The oligomeric duplex of any one of embodiments A89, A90 and A92, wherein the 5′-terminal nucleoside of the second modified oligonucleotide comprises a 2′-OMe sugar moiety and/or the nucleoside immediately 3′ of the 5′-terminal nucleoside of the second modified oligonucleotide comprises a 2′-OMe sugar moiety.

Embodiment A94. The oligomeric duplex of embodiment A89 or embodiment A90, wherein the 5′-terminal nucleoside, the nucleoside immediately 3′ of the 5′-terminal nucleoside, the 3′-terminal nucleoside, and the nucleoside immediately 5′ of the 3′-terminal nucleoside of the second modified oligonucleotide comprises a 2′-OMe sugar moiety.

Embodiment A95. The oligomeric duplex of any one of embodiments A1-A94, wherein one or more of the nucleosides of the second modified oligonucleotide comprise a 2′-MOE sugar moiety.

Embodiment A96. The oligomeric duplex of any one of embodiments A1-A93, wherein the 3′-terminal nucleoside of the second modified oligonucleotide comprises a 2′-MOE sugar moiety and/or the nucleoside immediately 5′ of the 3′-terminal nucleoside of the second modified oligonucleotide comprises a 2′-MOE sugar moiety.

Embodiment A97. The oligomeric duplex of any one of embodiments A1-A93, A95 and A96, wherein the 5′-terminal nucleoside of the second modified oligonucleotide comprises a 2′-MOE sugar moiety and/or the nucleoside immediately 3′ of the 5′-terminal nucleoside of the second modified oligonucleotide comprises a 2′-MOE sugar moiety.

Embodiment A98. The oligomeric duplex of any one of embodiments A1-A91, wherein the 5′-terminal nucleoside, the nucleoside immediately 3′ of the 5′-terminal nucleoside, the 3′-terminal nucleoside, and the nucleoside immediately 5′ of the 3′-terminal nucleoside of the second modified oligonucleotide comprises a 2′-MOE sugar moiety.

Embodiment A99. The oligomeric duplex of any one of embodiments A1-A98, wherein the second modified oligonucleotide has a sugar motif (5′ to 3′) selected from: eeyyyyfyfyfyyyyyyyyee, eeyyyyfyfyfyyyyyyyyyy, yyyyyyfyfyfyyyyyyyyee, yyyyyyfyfyfyyyyyyyyyy, eeyyyyfyfffyyyyyyyyee, eeyyyyfyfffyyyyyyyyyy, yyyyyyfyfffyyyyyyyyee, yyyyyyfyfffyyyyyyyyyy, eeyyyyyyyffyyyyyyyyee, eeyyyyyyyffyyyyyyyyyy, yyyyyyyyyffyyyyyyyyee, yyyyyyyyyffyyyyyyyyyy, eeyyyyyffyyyyyyyyee, eeyyyyyffyyyyyyyyyy, yyyyyyyffyyyyyyyyee, yyyyyyyffyyyyyyyyyy, yyyyyyfyfffyyyyyyyy, eeyyyyfyfffyyyyyyee, eeyyyyfyfffyyyyyyyy, yyyyyyfyfffyyyyyyee, eeyyfyfffyyyyyyyyee, eeyyfyfffyyyyyyyyyy, yyyyfyfffyyyyyyyyee, yyyyfyfffyyyyyyyyyy, eeyyyyyyyydyyyyyyyyee, eeyyyyyyyydyyyyyyyyyy, yyyyyyyyyydyyyyyyyyee, yyyyyyyyyydyyyyyyyyyy, eeyyyyyyyyyyyyyyyyyee, eeyyyyyyyyyyyyyyyyyyy, yyyyyyyyyyyyyyyyyyyee, and yyyyyyyyyyyyyyyyyyyyy wherein each “y” represents a 2′-OMe sugar moiety, each “e” represents a 2′-MOE sugar moiety, each “f” represents a 2′-fluoro sugar moiety, and each “d” represents a 2′-β-D-deoxyribosyl sugar moiety.

Embodiment A100. The oligomeric duplex of any one of embodiments A1-A98, wherein the second modified oligonucleotide has a sugar motif (5′ to 3′) selected from: eeyyyyyyyffyyyyyyyyee, eeyyyyfyfyfyyyyyyyyee, yyyyyyyyyffyyyyyyyyyy, and yyyyyyfyfyfyyyyyyyyyy, wherein each “y” represents a 2′-OMe sugar moiety, each “e” represents a 2′-MOE sugar moiety, and each “f” represents a 2′-fluoro sugar moiety.

Embodiment A101. The oligomeric duplex of any one of embodiments A1-A100, wherein the nucleobase of the 3′-terminal nucleoside and/or of the nucleoside immediately 5′ of the 3′-terminal nucleoside of the second modified oligonucleotide is an adenine.

Embodiment A102. The oligomeric duplex of any one of embodiments A1-A100, wherein the nucleobase of the 3′-terminal nucleoside is an adenine and the nucleobase of the nucleoside immediately 5′ of the 3′-terminal nucleoside of the second modified oligonucleotide is a thymine or uracil.

Embodiment A103. The oligomeric duplex of any one of embodiments A1-A100, wherein the second modified oligonucleotide comprises at least one modified internucleoside linkage.

Embodiment A104. The oligomeric duplex of embodiment A103, wherein at least one modified internucleoside linkage is a phosphorothioate or mesyl phosphoramidate internucleoside linkage.

Embodiment A105. The oligomeric duplex of embodiment A103, wherein fewer than 50%, fewer than 40%, fewer than 30%, fewer than 20%, or fewer than 10% of the internucleoside linkages of the first modified oligonucleotide are modified internucleoside linkages.

Embodiment A106. The oligomeric duplex of any one of embodiments A103-A105, wherein each internucleoside linkage of the second modified oligonucleotide is independently selected from a phosphodiester internucleoside linkage and a phosphorothioate internucleoside linkage.

Embodiment A107. The oligomeric duplex of any one of embodiments A103-A105, wherein the internucleoside linkages between the first and second nucleosides and the second and third nucleosides counting from the 5′ end of the second modified oligonucleotide are modified internucleoside linkages.

Embodiment A108. The oligomeric duplex of any one of embodiments A103-A105 and A107, wherein the internucleoside linkages between the first and second nucleosides and the second and third nucleosides counting from the 3′ end of the first modified oligonucleotide are modified internucleoside linkages.

Embodiment A109. The oligomeric duplex of any one of embodiments A103-A105, wherein the internucleoside linkages between the first and second nucleosides and the second and third nucleosides counting from the 5′ end of the second modified oligonucleotide, and the internucleoside linkages between the first and second nucleosides and the second and third nucleosides counting from the 3′ end of the second modified oligonucleotide are modified internucleoside linkages.

Embodiment A110. The oligomeric duplex of any one of embodiments A103-A109, wherein the modified internucleoside linkages are phosphorothioate internucleoside linkages.

Embodiment A111. The oligomeric duplex of embodiment A110, wherein all other internucleoside linkages in the second modified oligonucleotide are phosphodiester internucleoside linkages.

Embodiment A112. The oligomeric duplex of embodiment A111, wherein the second modified oligonucleotide has an internucleoside linkage motif of ssooooooooooooooooss and a sugar motif (5′ to 3′) selected from: eeyyyyyyyffyyyyyyyyee, eeyyyyfyfyfyyyyyyyyee, yyyyyyyyyffyyyyyyyyyy, and yyyyyyfyfyfyyyyyyyyyy; wherein each ““y” represents a 2′-OMe sugar moiety, each “e” represents a 2′-MOE sugar moiety, each “f” represents a 2′-fluoro sugar moiety, each “o” represents a phosphodiester internucleoside linkage and each “s” represents a phosphorothioate internucleoside linkage.

Embodiment A113. The oligomeric duplex of any of embodiments A1-A112, wherein the nucleobase sequence of the second modified oligonucleotide comprises least 8, at least 9, at least 10, at least 11, at least 12, at least 13, at least 14, at least 15, at least 16, at least 17, at least 18, at least 19, at least 20, or at least 21 contiguous nucleobases of any of the nucleobase sequences of any of SEQ ID NOs: 315-626, 783-938, 977-1016, and 1027-1032.

Embodiment A114. The oligomeric duplex of any of embodiments A1-A112, wherein the nucleobase sequence of the second modified oligonucleotide comprises the nucleobase sequence of any of SEQ ID NOs: 315-626, 783-938, 977-1016, and 1027-1032.

Embodiment A115. The oligomeric duplex of any of embodiments A1-A112, wherein the nucleobase sequence of the second modified oligonucleotide consists of the nucleobase sequence of any of SEQ ID NOs: 315-626, 783-938, 977-1016, and 1027-1032.

Embodiment A116. The oligomeric duplex of any one of embodiments A1-A112, wherein the nucleobase sequence of the second modified oligonucleotide comprises at least 8, at least 9, at least 10, at least 11, at least 12, at least 13, at least 14, at least 15, or at least 16 contiguous nucleobases of any of the nucleobase sequences of SEQ ID NOs: 341, 483, 488, 1011-1016, and 1027-1032.

Embodiment A117. The oligomeric duplex of any of embodiments A1-A112, wherein the nucleobase sequence of the second modified oligonucleotide comprises the nucleobase sequence of any of SEQ ID NOs: 341, 483, 488, 1011-1016, and 1027-1032.

Embodiment A118. The oligomeric duplex of any of embodiments A1-A112, wherein the nucleobase sequence of the second modified oligonucleotide consists of the nucleobase sequence of any of SEQ ID NOs: 341, 483, 488, 1011-1016, and 1027-1032.

Embodiment A119. An oligomeric duplex comprising:
(1) a first oligomeric compound comprising a first modified oligonucleotide consisting of 18 to 30 contiguous linked nucleosides that is at least 85%, at least 90%, at least 95% or at least 95% complementary or 100% complementary to an equal length portion within nucleobases 295-316, 296-317, 297-318, 298-320, 299-320, 300-321, 301-322, 302-324, 303-324, 304-325, 304-326, 305-327, 306-325, 306-328, 307-328, 308-329, 309-330, 310-331, 311-332, 312-333, 313-334, 314-335, 315-336, 316-337, 317-339, 318-339, 319-340, 320-341, 321-342, 322-344, 323-345, 324-345, 324-346, 325-345, 325-347, 326-347, 327-348, 328-349, 329-350, 330-351, 331-352, 332-353, 333-354, 334-355, 505-526, 506-528, 507-529, 508-530, 509-531, 510-531, 511-532, 512-533, 513-535, 514-535, 514-536, 515-536, 516-535, 516-537, 517-538, 518-539, 519-541, 520-541, 521-542, 522-544, 523-544, 524-546, 535-556, 536-557, 537-558, 538-559, 539-560, 540-561, 541-563, 542-563, 543-564, 544-565, 545-566, 546-567, 547-568, 548-570, 549-571, 550-572, 551-572, 552-574, 553-574, 554-575, 555-577, 556-578, 557-579, 558-580, 559-580, 560-581, 561-582, 562-583, 563-584, 595-616, 596-618, 597-618, 598-620, 599-621, 600-622, 601-623, 602-623, 603-624, 604-625, 605-627, 606-628, 607-628, 608-629, 609-630, 610-631, 611-632, 612-633, 613-635, 665-687, 666-687, 667-689, 668-689, 669-690, 670-691, 671-692, 672-694, 673-694, 674-695, 675-696, 676-697, 677-698, 678-700, 679-701, 680-702, 681-703, 682-703, 683-704, 684-705, 685-706, 686-708, 687-708, 688-709, 689-710, 690-711, 691-712, 692-714, 693-715, 1675-1696, 1676-1698, 1677-1698, 1678-1699, 1679-1700, 1680-1702, 1681-1702, 1682-1703, 1683-1705, 1684-1705, 1685-1706, 1686-1707, 1687-1709, 1688-1709, 1689-1710, 1690-1712, 1691-1713, 1692-1714, and 1693-1714 of SEQ ID NO: 1, and
(2) a second oligomeric compound comprising a second modified oligonucleotide consisting of 16 to 28 contiguous linked nucleosides that is at least 90% complementary to an equal length portion of the first modified oligonucleotide;
   wherein:
(a) each of the nucleosides of the first modified oligonucleotide and each of the nucleosides of the second modified oligonucleotide comprise a modified sugar moiety or a sugar surrogate, (b) at least one of the modified sugar moiety or sugar surrogate of the first modified oligonucleotide comprises a fluorine, and
(c) fewer than 40% of the nucleosides of the first modified oligonucleotide comprise a fluorine.

Embodiment A120. The oligomeric duplex of embodiment A119, wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases of any of the nucleobase sequences of any of SEQ ID NOs: 3-314, 627-782, 939-976, 1033-1038 and 1254-1255.

Embodiment A121. The oligomeric duplex of embodiment A119, wherein the nucleobase sequence of the first modified oligonucleotide comprises the nucleobase sequence of any of SEQ ID NOs: 3-314, 627-782, 939-976, 1033-1038, and 1254-1255.

Embodiment A122. The oligomeric duplex of embodiment A119, wherein the nucleobase sequence of the first modified oligonucleotide consists of the nucleobase sequence of any of SEQ ID NOs: 3-314, 627-782, 939-976, 1033-1038, and 1254-1255.

Embodiment A123. The oligomeric duplex of embodiment A119, wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases of any of the nucleobase sequences of SEQ ID NOs: 29, 171, 176, 967, 968, 971, 972, 974, 975, 1254, 1255, and 1033-1038.

Embodiment A124. The oligomeric duplex of embodiment A119, wherein the nucleobase sequence of the first modified oligonucleotide comprises the nucleobase sequence of any of SEQ ID NOs: 29, 171, 176, 967, 968, 971, 972, 974, 975, 1254, 1255, and 1033-1038.

Embodiment A125. The oligomeric duplex of embodiment A119, wherein the nucleobase sequence of the first modified oligonucleotide consists of the nucleobase sequence of any of SEQ ID NOs: 29,171,176, 967, 968, 971, 972, 974, 975, 1254, 1255, and 1033-1038.

Embodiment A126. The oligomeric duplex of embodiment A120, wherein the nucleobase sequence of the second modified oligonucleotide comprises least 8, at least 9, at least 10, at least 11, at least 12, at least 13, at least 14, at least 15, at least 16, at least 17, at least 18, at least 19, at least 20, or at least 21 contiguous nucleobases of any of the nucleobase sequences of any of SEQ ID NOs: 315-626, 783-938, 977-1016, and 1027-1032.

Embodiment A127. The oligomeric duplex of embodiment A121, wherein the nucleobase sequence of the second modified oligonucleotide comprises the nucleobase sequence of any of SEQ ID NOs: 315-626, 783-938, 977-1016, and 1027-1032.

Embodiment A128. The oligomeric duplex of embodiment A122, wherein the nucleobase sequence of the second modified oligonucleotide consists of the nucleobase sequence of any of SEQ ID NOs: 315-626, 783-938, 977-1016, and 1027-1032.

Embodiment A129. The oligomeric duplex of embodiment A123, wherein the nucleobase sequence of the second modified oligonucleotide comprises at least 8, at least 9, at least 10, at least 11, at least 12, at least 13, at least 14, at least 15, at least 16, at least 17, at least 18, at least 19, at least 20, or at least 21 contiguous nucleobases of any of the nucleobase sequences of SEQ ID NOs: 341, 483, 488, 1011-1016, and 1027-1032.

Embodiment A130. The oligomeric duplex of embodiment A124, wherein the nucleobase sequence of the second modified oligonucleotide comprises the nucleobase sequence of any of SEQ ID NOs: 341, 483, 488, 1011-1016, and 1027-1032.

Embodiment A131. The oligomeric duplex of embodiment A125, wherein the nucleobase sequence of the second modified oligonucleotide consists of the nucleobase sequence of any of SEQ ID NOs: 341, 483, 488, 1011-1016, and 1027-1032.

Embodiment A132. The oligomeric duplex of any one of embodiments A119-A131, wherein none of the modified sugar moieties of the nucleosides of the second modified oligonucleotide comprises a fluorine.

Embodiment A133. The oligomeric duplex of any one of embodiments A119-A131, wherein at least one of the modified sugar moieties of the second modified oligonucleotide comprises a fluorine and wherein fewer than 40% of the nucleosides of the second modified oligonucleotide comprises a fluorine.

Embodiment A134. The oligomeric duplex of any one of embodiments A119-A131, wherein two, three or four of the nucleosides of the second modified oligonucleotide comprise a modified sugar moiety comprising a fluorine.

Embodiment A135. The oligomeric duplex of any one of embodiments A119-A134, wherein two, three or four of the nucleosides of the first modified oligonucleotide comprise a modified sugar moiety comprising a fluorine.

Embodiment A136. The oligomeric duplex of any one of embodiments A119-A135 wherein none of the nucleosides of the second modified oligonucleotide is a 2′-deoxynucleoside.

Embodiment A137. The oligomeric duplex of any one of embodiments A119-A135 wherein all of the nucleosides of the second modified oligonucleotide comprise a modified ribosyl sugar moiety.

Embodiment A138. The oligomeric duplex of any one of embodiments A119-A137, wherein one, two or three of the nucleosides of the first modified oligonucleotide is/are a 2′-deoxynucleoside.

Embodiment A139. The oligomeric duplex of any one of embodiments A119-A138, wherein:
(a) the 5′- and 3′-terminal nucleosides, the nucleoside immediately adjacent to the 5′-terminal nucleoside, and the nucleoside immediately adjacent to the 3′-terminal nucleoside of the second modified oligonucleotide comprise a 2′-MOE sugar moiety,
(b) the 5′- and 3′-terminal nucleosides and the nucleoside immediately 5′ of the 3′-terminal nucleoside of the first modified oligonucleotide comprise a 2′-MOE sugar moiety, and/or
(b) any nucleoside in the first and second modified oligonucleotides that does not comprise a fluorine or a 2′-MOE sugar moiety comprises a 2′-OMe sugar moiety.

Embodiment A140. The oligomeric duplex of any one of embodiments A119-A139, wherein no more than four of the nucleosides of the first modified oligonucleotide comprise a modified sugar moiety comprising a fluorine.

Embodiment A141. The oligomeric duplex of embodiment A140, wherein the first modified oligonucleotide has a sugar motif (5′ to 3′) selected from: efyyyfyyyyyyyfyfyyyyyee, efyyyfyyyyyyyfyfyyyyyyy, e[FHNA]yyyfyyyyyyyfyfyyyyyee, e[FHNA]yyyfyyyyyyyfyfyyyyyyy, efyyyfyyyyyyyfyfyyyyyey, e[FHNA]yydydyyyyfyfyfyyyyyyy and efyyyfyyyyyyyfyfyyyee; wherein each “d” represents a 2′-β-D-deoxyribosyl sugar moiety, each “y” represents a 2′-OMe sugar moiety, each “e” represents a 2′-MOE sugar moiety, each “f” represents a 2′-fluoro sugar moiety, and each “[FHNA]” represents a 3′-fluoro-hexitol sugar moiety.

Embodiment A142. The oligomeric duplex of any one of embodiments A119-A139, wherein no more than three of the nucleosides of the first modified oligonucleotide comprise a modified sugar moiety comprising a fluorine.

Embodiment A143. The oligomeric duplex of embodiment A142 wherein the first modified oligonucleotide has a sugar motif (5′ to 3′) selected from: efyyydyyyyyyyfyfyyyyyee, efyyydyyyyyyyfyfyyyyyyy, e[FHNA]yyydyyyyyyyfyfyyyyyee, e[FHNA]yyydyyyyyyyfyfyyyyyyy, efyydydyyyyyyfyfyyyyyee, efyydydyyyyyyfyfyyyyyyy, e[FHNA]yydydyyyyyyfyfyyyyyee, e[FHNA]yydydyyyyyyfyfyyyyyyy, efyyyyyyyyyyfyfyyyyyee, efyyyfyyyyyyyfyyyyyyyyy, efyyyfyyyyyyyfydyyyyyee, efyyyfyyyyyyyfydyyyyyyy, efyyyfyyyyyyyfydyyyyyee, efyyyfyyyyyyyfydyyyyyyy; wherein each “d” represents a 2′-β-D-deoxyribosyl sugar moiety, each “y” represents a 2′-OMe sugar moiety, each “e” represents a 2′-MOE sugar moiety, each “f” represents a 2′-fluoro sugar moiety, and each “[FHNA]” represents a 3′-fluoro-hexitol sugar moiety.

Embodiment A144. The oligomeric duplex of any one of embodiments A119-A139, wherein no more than two of the nucleosides of the first modified oligonucleotide comprise a modified sugar moiety comprising a fluorine.

Embodiment A145. The oligomeric duplex of embodiment A144 wherein the first modified oligonucleotide has a sugar motif (5′ to 3′) selected from: efyyyyyyyyyyyfyyyyyyyee, efyyyyyyyyyyyfyyyyyyyyy, e[FHNA]yyyyyyyyyyy[FHNA]yyyyyyyee, and e[FHNA]yyyyyyyyyyy[FHNA]yyyyyyyyy; wherein each “y” represents a 2′-OMe sugar moiety, each “e” represents a 2′-MOE sugar moiety, each “f” represents a 2′-fluoro sugar moiety, and each “[FHNA]” represents a 3′-fluoro-hexitol sugar moiety.

Embodiment A146. The oligomeric duplex of any one of embodiments A119-A139, wherein only one of the nucleosides of the first modified oligonucleotide comprise a modified sugar moiety comprising a fluorine.

Embodiment A147. The oligomeric duplex of embodiment A146 wherein the first modified oligonucleotide has a sugar motif (5′ to 3′) selected from: efyyydyyyyyyydydyyyyyyy, efyyydyyyyyyydydyyyyyee, e[FHNA]yyydyyyyyyydydyyyyyyy, and e[FHNA]yyydyyyyyyydydyyyyyee; wherein each “d” represents a 2′-β-D-deoxyribosyl sugar moiety, each “y” represents a 2′-OMe sugar moiety, each “e” represents a 2′-MOE sugar moiety, and each “f′ represents a 2′-fluoro sugar moiety.

Embodiment A148. The oligomeric duplex of any one of embodiments A119-A146, wherein no more than two of the nucleosides of the first modified oligonucleotide are 2′-deoxynucleosides.

Embodiment A149. The oligomeric duplex of embodiment A148 wherein the first modified oligonucleotide has a sugar motif (5′ to 3′) selected from: efyydydyyyyyyfyfyyyyyee, efyydydyyyyyyfyfyyyyyyy, e[FHNA]yydydyyyyyyfyfyyyyyee, and e[FHNA]yydydyyyyyyfyfyyyyyyy; wherein each “d” represents a 2′-β-D-deoxyribosyl sugar moiety, each “y” represents a 2′-OMe sugar moiety, each “e” represents a 2′-MOE sugar moiety, each “f” represents a 2′-fluoro sugar moiety, and each “[FHNA]” represents a 3′-fluoro-hexitol sugar moiety.

Embodiment A150. The oligomeric duplex of any one of embodiments A119-A147, wherein only one of the nucleosides of the first modified oligonucleotide is a 2′-deoxynucleoside.

Embodiment A151. The oligomeric duplex of embodiment A150, wherein the first modified oligonucleotide has a sugar motif (5′ to 3′) selected from: efyyydyyyyyyyfyfyyyyyee, efyyydyyyyyyyfyfyyyyyyy, e[FHNA]yyydyyyyyyyfyfyyyyyee, e[FHNA]yyydyyyyyyyfyfyyyyyyy, eyyyfyyyyyyyfydyyyyyyy, efyyyfyyyyyyyfydyyyyyee, eyyy[FHNA]yyyyyyyfydyyyyyyy, and e[FHNA]yyyfyyyyyyyfydyyyyyee; wherein each “d” represents a 2′-β-D-deoxyribosyl sugar moiety, each “y” represents a 2′-OMe sugar moiety, each “e” represents a 2′-MOE sugar moiety, each “f′ represents a 2′-fluoro sugar moiety, and each “[FHNA]” represents a 3′-fluoro-hexitol sugar moiety.

Embodiment A152. The oligomeric duplex of any one of embodiments A119-A151, wherein no more than four of the nucleosides of the second modified oligonucleotide comprise a modified sugar moiety comprising a fluorine.

Embodiment A153. The oligomeric duplex of embodiment A152, wherein the second modified oligonucleotide has a sugar motif (5′ to 3′) selected from: yyyyyyfyfffyyyyyyyyyy, eeyyyyfyfffyyyyyyyyee, yyyyyyfyfffyyyyyyyy, eeyyyyfyfffyyyyyyee, yyyyfyfffyyyyyyyyyy, and eeyyfyfffyyyyyyyyee; wherein each “y” represents a 2′-OMe sugar moiety, each “e” represents a 2′-MOE sugar moiety, and each “f” represents a 2′-fluoro sugar moiety.

Embodiment A154. The oligomeric duplex of any one of embodiments A119-A151, wherein no more than three of the nucleosides of the second modified oligonucleotide comprise a modified sugar moiety comprising a fluorine.

Embodiment A155. The oligomeric duplex of embodiment A154, wherein the second modified oligonucleotide has a sugar motif (5′ to 3′) selected from: eeyyyyfyfyfyyyyyyyyee and yyyyyfyfyfyyyyyyyyy; wherein each “y” represents a 2′-OMe sugar moiety, each “e” represents a 2′-MOE sugar moiety, and each “f” represents a 2′-fluoro sugar moiety.

Embodiment A156. The oligomeric duplex of any one of embodiments A119-A152, wherein no more than two of the nucleosides of the second modified oligonucleotide comprise a modified sugar moiety comprising a fluorine.

Embodiment A157. The oligomeric duplex of embodiment A156, wherein the second modified oligonucleotide has a sugar motif (5′ to 3′) selected from: eeyyyyyyyffyyyyyyyyee, yyyyyyyyyffyyyyyyyyyy, eeyyyyyffyyyyyyyyee, and yyyyyyyffyyyyyyyyyy; wherein each “y” represents a 2′-OMe sugar moiety, each “e” represents a 2′-MOE sugar moiety, and each “f” represents a 2′-fluoro sugar moiety.

Embodiment A158. The oligomeric duplex of any one of embodiments A119-A151, wherein none of the nucleosides of the second modified oligonucleotide comprises a modified sugar moiety comprising a fluorine.

Embodiment A159. The oligomeric duplex of embodiment A158, wherein the second modified oligonucleotide has a sugar motif (5′ to 3′) selected from: yyyyyyyyyyyyyyyyyyyyy, eeyyyyyyyyyyyyyyyyyee, yyyyyyyyyydyyyyyyyyyy, and eeyyyyyyyydyyyyyyyyee.

Embodiment A160. The oligomeric duplex of any one of embodiments A119-A159, wherein fewer than 50%, fewer than 40%, fewer than 30%, fewer than 20%, or fewer than 10% of the internucleoside linkages of the first modified oligonucleotide are modified internucleoside linkages.

Embodiment A161. The oligomeric duplex of any one of embodiments A119-A160, wherein the internucleoside linkages between the first and second nucleosides and the second and third nucleosides counting from the 5′ end of the first modified oligonucleotide, and the internucleoside linkages between the first and second nucleosides and the second and third nucleosides counting from the 3′ end of the first modified oligonucleotide are phosphorothioate internucleoside linkages.

Embodiment A162. The oligomeric duplex of any one of embodiments A119-A161, wherein the first modified oligonucleotide has an internucleoside linkage motif (5′ to 3′) selected from: ssooooooooooooooooooss, ssooosooooooooooooooss, ssoososoooooooooooooss, and ssooooooooooooooooss; wherein each “o” represents a phosphodiester internucleoside linkage and each “s” represents a phosphorothioate internucleoside linkage.

Embodiment A163. The oligomeric duplex of any one of embodiments A119-A162, wherein fewer than 50%, fewer than 40%, fewer than 30%, fewer than 20%, or fewer than 10% of the internucleoside linkages of the second modified oligonucleotide are modified internucleoside linkages.

Embodiment A164. The oligomeric duplex of any one of embodiments A119-A163, wherein the internucleoside linkages between the first and second nucleosides and the second and third nucleosides counting from the 5′ end of the second modified oligonucleotide, and the internucleoside linkages between the first and second nucleosides and the second and third nucleosides counting from the 3′ end of the second modified oligonucleotide are phosphorothioate internucleoside linkages.

Embodiment A165. The oligomeric duplex of any one of embodiments A119-A164, wherein the second modified oligonucleotide has an internucleoside linkage motif (5′ to 3′) selected from: ssooooooooooooooooss, ssooooooosooooooooss, ssoooooozozoooooooss, and ssooooooooooooooss; wherein each “o” represents a phosphodiester internucleoside linkage, each “s” represents a phosphorothioate internucleoside linkage, and each ‘z’ represents a mesyl phosphoramidate internucleoside linkage.

Embodiment A166. The oligomeric duplex of any one of embodiments A1-A165, wherein the first modified oligonucleotide has a first sugar motif (1^{st}) and the second modified oligonucleotide has a second sugar motif (2^{nd}) and the first and second sugar motifs are selected from among the following combinations (5′ to 3′): 1^{st}: efyydydyyyyyyfyfyyyyyee and 2^{nd}: eeyyyyfyfyfyyyyyyyyee; 1^{st}: efyydydyyyyyyfyfyyyyyyy and 2^{nd}: yyyyyyfyfyfyyyyyyyyyy; 1^{st}: efyydydyyyyyyfyfyyyyyee and 2^{nd}: yyyyyyfyfyfyyyyyyyyyy; 1^{st}: efyydydyyyyyyfyfyyyyyyy and 2^{nd}: eCeyyyyffyfyyyyyyyyee; 1^{st}: efyyydyyyyyyyfyfyyyyyee and 2^{nd}: eeyyyyyyyffyyyyyyyyee; 1^{st}: efyyydyyyyyyyfyfyyyyyyy and 2^{nd}: yyyyyyyyyffyyyyyyyyyy; 1^{st}: efyyydyyyyyyyfyfyyyyyee and 2^{nd}: yyyyyyyyyffyyyyyyyyyy; 1^{st}: efyyydyyyyyyyfyfyyyyyyy and 2^{nd}: eeyyyyyyyffyyyyyyyyee; 1^{st}: efyydydyyyyyyfyfyyyyyee and 2^{nd}: eeyyyyyyyffyyyyyyyyee; 1^{st}: efyydydyyyyyyfyfyyyyyyy and 2^{nd}: yYYYYYYYYffYYYYyyyyyy; 1^{st}: efyydydyyyyyyfyfyyyyyee and 2^{nd}: yyyyyyyffyyyyyyyy; 1^{st}: efyydydyyyyyyfyfyyyyyyy and 2^{nd}: eeyyyyyyyffyyyyyyyyee; 1^{st}: efyyydyyyyyyyfyfyyyyyee and 2^{nd}: eeyyyyfyfyfyyyyyyyyee; 1^{st}: efyyydyyyyyyyfyfyyyyyyy and 2^{nd}: yyyyyyfyfyfyyyyyyyyyy; 1^{st}: efyyydyyyyyyyfyfyyyyyee and 2^{nd}: yyyyyyfyfyfyyyyyyyyyy; and 1^{st}: efyyydyyyyyyyfyfyyyyyyy and 2^{nd}: eeyyyyfyfyfyyyyyyyyee, wherein each “y” represents a 2′-OMe sugar moiety, each “e” represents a 2′-MOE sugar moiety, each “f” represents a 2′-fluoro sugar moiety, and each “d” represents a 2′-β-D-deoxyribosyl sugar moiety.

Embodiment A167. The oligomeric duplex of any one of embodiments A1-A165, wherein the first modified oligonucleotide has a first sugar motif (1′t) and the second modified oligonucleotide has a second sugar motif (2^{nd}) and the first and second sugar motifs are selected from among the following combinations (5′ to 3′): 1^{st}: efyydydyyyyyyfyfyyyyyee and 2^{nd}: eeyyyyfyfffyyyyyyyyee; 1^{st}: efyydydyyyyyyfyfyyyyyyy and 2^{nd}: yyyyyyfyfffyyyyyyyyyy; 1^{st}: efyydydyyyyyyfyfyyyyyee and 2^{nd}: yyyyyyfyfffyyyyyyyyyy; 1^{st}: efyydydyyyyyyfyfyyyyyyy and 2^{nd}. eeyyyyfyfffyyyyyyyyee; 1^{st}: efyyyyyyyyyyyfyfyyyyyee and 2^{nd}: eeyyyyfyfffyyyyyyyyee; 1^{st}: efyyyyyyyyyyyfyfyyyyyyy and 2^{nd}: yyyyyyfyfffyyyyyyyyyy; 1^{st}: efyyyyyyyyyyfyfyyyyyee and 2^{nd}: yyyyyyfyfffyyyyyyyyyy; 1^{st}: efyyyyyyyyyyyfyfyyyyyyy and 2^{nd}: eeyyyyfyfffyyyyyyyyee; 1^{st}: efyyyyyyyyyyyfyfyyyyyee and 2^{nd}: eeyyyyyyyffyyyyyyyyee; 1^{st}: efyyyyyyyyyyyfyfyyyyyyy and 2^{nd}: yyyyyyyyyffyyyyyyyyyy; 1^{st}: efyyyyyyyyyyyfyfyyyyyee and 2^{nd}: yyyyyyyyffyyyyyyyyyy; 1^{st}: efyyyyyyyyyyyfyfyyyyyyy and 2^{nd}: eeyyyyyyyffyyyyyyyyee; 1^{st}: efyyyyyyyyyyyfyfyyyyyee and 2^{nd}: eeyyyyfyffyyyyyyyyee; 1^{st}: efyyyyyyyyyyyfyfyyyyyyy and 2^{nd}: yyyyyfyfyfyyyyyyyyyy; 1^{st}: efyyyyyyyyyyyfyfyyyyyee and 2^{nd}: yyyyyyfyfyfyyyyyyyyyy; 1^{st}: efyyyyyyyyyyyfyfyyyyyyy and 2^{nd}: eeyyyyfyfyfyyyyyyyyee; 1^{st}: efyyyfyyyyyyyfyfyyyyyyy and 2^{nd}: yyyyyyffyyyyyyyyyy; 1^{st}: efyyyfyyyyyyyfyfyyyyyee and 2^{nd}: eeyyyyyyyffyyyyyyyyee; 1^{st}: efyyyfyyyyyyyfyfyyyyyyy and 2^{nd}: eeyyyyyyffyyyyyyyyee; 1^{st}: efyyyfyyyyyyyfyfyyyyyee and 2^{nd}: yy yyffyyyyyyyyyy; 1^{st}: efyyyfyyyyyyyfyfyyyyyyy and 2^{nd}: yyyyyyfyfyfyyyyyyyyyy; 1^{st}: efyyyfyyyyyyyyfyyyyyee and 2^{nd}: eeyyyyfyfyfyyyyyyyyee; 1^{st}: efyyyfyyyyyyyfyfyyyyyyy and 2^{nd}: eeyyyyfyfyfyyyyyyyyee; 1^{st}: efyyyfyyyyyyyfyfyyyyyee and 2^{nd}: yyyyyyfyfyfyyyyyyyyyy; 1^{st}: efyyyfyyyyyyyfyfyyyee and 2^{nd}: eeyyyyfyfffyyyyyyee; 1^{st}: efyyyfyyyyyyyfyfyyyyy and 2^{nd}: yyyyyyfyfffyyyyyyyy; 1^{st}: efyyyfyyyyyyyfyfyyyee and 2^{nd}: yyyyyyfyfffyyyyyyyy; 1^{st}: efyyyfyyyyyyyfyfyyyyy and 2^{nd}: eeyyyyfyfffyyyyyyee; 1^{st}: efyyyfyyyyyyyfyfyyyee and 2^{nd}: eeyyyyffyyyyyyyyee; 1^{st}: efyyyfyyyyyyyfyfyyyyy and 2^{nd}: yyyyyyffyyyyyyyyyy; 1^{st}: efyyyfyyyyyyyfyfyyyee and 2^{nd}: yyyyyffyyyyyyyyyY 1^{st}: efyyyfyyyyyyyfyfyyyyy and 2^{nd}: eeyyyyffyyyyyyyyee; 1^{st}: e[FHNA]yydydyyyyfyfyfyyyyyee and 2^{nd}: eeyyyyfyfffyyyyyyyyee; 1^{st}: efyyyfyyyyyyyfyfyyyyyey and 2^{nd}: eeyyyyfyfffyyyyyyyyee; 1^{st}: efyyyfyyyyyyyfyfyyyyyey and 2^{nd}: yyyyyyfyfffyyyyyyyyyy; 1^{st}: e[FHNA]yydydyyyyfyfyfyyyyyyy and 2^{nd}: yyyyyfyfffyyyyyyyyyy; 1^{st}: e[FHNA]yydydyyyyfyfyfyyyyyee and 2^{nd}: yyyyyyfyfffyyyyyyyyyy; 1¹: e[FHNA]yydydyyyyfyfyfyyyyyyy and 2^{nd}: eeyyyyfyfffyyyyyyyyee; 1^{st}: e[FHNA]yydydyyyyfyfyfyyyyyee and 2^{nd}: eeyyyyfyfyfyyyyyyyyee; 1^{st}: e[FHNA]yydydyyyyfyfyfyyyyyyy and 2^{nd}: yyyyyyfyfyfyyyyyyyyyy; 1^{st}: e[FHNA]yydydyyyyfyfyfyyyyyee and 2^{nd}: yyyyyyfyfyfyyyyyyyyyy; and 1^{st}: e[FHNA]yydydyyyyfyfyfyyyyyyy and 2^{nd}: eeyyyyfyfyfyyyyyyyyee, wherein each “y” represents a 2′-OMe sugar moiety, each “e” represents a 2′-MOE sugar moiety, each “f” represents a 2′-fluoro sugar moiety, each “[FHNA]” represents a 3′-fluoro-hexitol sugar moiety, and each “d” represents a 2′-D-D-deoxyribosyl sugar moiety.

Embodiment A168. The oligomeric duplex of any one of embodiments A1-A165, wherein the first modified oligonucleotide has a first sugar motif (1′t) and the second modified oligonucleotide has a second sugar motif (2^{nd}) and the first and second sugar motifs are selected from among the following combinations (5′ to 3′): 1^{st}: efyyyfyyyyyyyfyfyyyyyyy and 2^{nd} yyyyyyfyfffyyyyyyyyyy; 1^{st}: efyyyfyyyyyyyfyfyyyyyee and 2^{nd}. eeyyyyfyfffyyyyyyyyee; 1^{st}: efyyyfyyyyyyyfyfyyyyyee and 2^{nd}. yyyyyyfyfffyyyyyyyyyy; and 1^{st}: efyyyfyyyyyyyfyfyyyyyyy and 2^{nd}: eeyyyyfyfffyyyyyyyyee, wherein each “y” represents a 2′-OMe sugar moiety, each “e” represents a 2′-MOE sugar moiety, and each “f” represents a 2′-fluoro sugar moiety.

Embodiment A169. The oligomeric duplex of any one of embodiments A1-A165, wherein the first modified oligonucleotide has a first sugar motif (1′) and the second modified oligonucleotide has a second sugar motif (2^{nd}) and the first and second sugar motifs are selected from among the following combinations (5′ to 3′): 1^{st}: efyyyfyyyyyyyfyfyyyyyyy and 2^{nd} yyyyyyyyyyyyy; 1^{st}: efyyyfyyyyyyyfyfyyyyyee and 2^{nd}. eeyyyyyyyyyyyyyyyyyee; 1^{st}: efyyyfyyyyyyyfyfyyyyyyy and 2^{nd}. eeyyyyyyyyyyyyyyyyyee; 1^{st}: efyyyfyyyyyyyfyfyyyyyee and 2^{nd}. yyyyyyyyyyyyyyyyyyyy; 1^{st}: efyyyfyyyyyyyfyfyyyyyyy and 2^{nd}: yyyyyyyyyydyyyyyyyyyy; 1^{st}: efyyyfyyyyyyyfyfyyyyyee and 2^{nd} eeyyyyyyyydyyyyyyyyee; 1^{st}: efyyyfyyyyyyyfyfyyyyyyy and 2^{nd}: eeyyyyyyyydyyyyyyyyee; 1^{st}: efyyyfyyyyyyyfyfyyyyyee and 2^{nd}: yyyyyyyyyydyyyyyyyyyy; 1^{st}: efyyyyyyyyyyyfyyyyyyyee and 2^{nd}. eeyyyyfyfffyyyyyyyyee; 1^{st}: efyyyyyyyyyyyfyyyyyyyyy and 2^{nd} yyyyyyfyfffyyyyyyyyyy; 1^{st}: efyyyyyyyyyyyfyyyyyyyee and 2^{nd}. yyyyyyfyfffyyyyyyyyyy; 1^{st}: efyyyyyyyyyyyfyyyyyyyyy and 2^{nd}. eeyyyyfyfffyyyyyyyyee; 1′t: efyyyyyyyyyyyfyyyyyyyee and 2^{nd}. eeyyyyfyfyfyyyyyyyyee; 11: efyyyyyyyyyyyfyyyyyyyyy and 2^{nd} yyyyyyfyfyfyyyyyyyyyy; 1^{st}: efyyyyyyyyyyyfyyyyyyyee and 2^{nd} yyyyyyfyfyfyyyyyyyyyy; 1^{st}: efyyyyyyyyyyyfyyyyyyyyy and 2^{nd} eeyyyyfyfyfyyyyyyce; 1^{st}: efyyydyyyyyyyfyfyyyyyee and 2^{nd} eeyyyyfyfffyyyyyyyyee; 1^{st}: efyyydyyyyyyyfyfyyyyyyy and 2^{nd} yyyyyyfyfffyyyyyyyyyy; 1^{st}: efyyydyyyyyyyfyfyyyyyee and 2^{nd}. yyyyyyfyfffyyyyyyyyyy; 1^{st}: efyyydyyyyyyyfyfyyyyyyy and 2^{nd} eeyyyyfyfffyyyyyyyyee; 1^{st}: efyyyfyyyyyyyfyfyyyee and 2^{nd} eeyyfyfffyyyyyyyyee; 1^{st}: efyyyfyyyyyyyfyfyyyyy and 2^{nd}. yyyyfyfffyyyyyyyyyy; 1^{st}: efyyyfyyyyyyyfyfyyyee and 2^{nd}. yyfyfffyyyyyyyyyy; 1^{st}: efyyyfyyyyyyyfyfyyyyy and 2^{nd}. eeyyfyfffyyyyyyyyee; 1^{st}: efyyyfyyyyyyyfydyyyyyee and 2^{nd} eeyyyfyfffyyyyyyyyee; 1^{st}: efyyyfyyyyyyyfydyyyyyyy and 2^{nd} yyyyyyfyfffyyyyyyyyyy; 1^{st}: efyyyfyyyyyyyfydyyyyyee and 2^{nd} yyyyyyfyfffyyyyyyyyyy; 1^{st}: efyyyfyyyyyyyfydyyyyyyy and 2^{nd}. eeyyyyfyfffyyyyyyyyee; 1^{st}: e[FHNA]yyyfyyyyyyyfyfyyyyyee and 2^{nd} eeyyyyfyfffyyyyyyyyee; 1^{st}: e[FHNA]yyyfyyyyyyyfyfyyyyyyy and 2^{nd} yyyyyyfyfffyyyyyyyyyy; 1^{st}: e[FHNA]yyyfyyyyyyyfyfyyyyyee and 2^{nd}: yyyyyyfyfffyyyyyyyyyy; and 1^{st}: e[FHNA]yyyfyyyyyyyfyfyyyyyyy and 2^{nd}: eeyyyyfyfffyyyyyyyyee, wherein each “y” represents a 2′—OMe sugar moiety, each “e” represents a 2′-MOE sugar moiety, each “f” represents a 2′-fluoro sugar moiety, each “[FHNA]” represents a 3′-fluoro-hexitol sugar moiety, and each “d” represents a 2′-D-D-deoxyribosyl sugar moiety.

Embodiment A170. The oligomeric duplex of any one of embodiments A1-A165, wherein the first modified oligonucleotide has a first sugar motif (1′t) and the second modified oligonucleotide has a second sugar motif (2^{nd}) and the first and second sugar motifs are selected from among the following combinations (5′ to 3′): 1^{st}: efyyyyyyyyyyyfyyyyyyyee and 2^{nd} eeyyy yffyyyyyyyyee; 1^{st}: efyyyyyy fyyyyyyyy and 2^{nd} yyyyyyyyyffyyyyyyyyyy 1^{st}: efyyyyyyyyyyyfyyyyyyyee and 2^{nd} yyyyyyyyyffyyyyyyyyyy; 1^{st}: efyyyyyyyyyyyfyyyyyyyyy and 2^{nd}. eeyyyyyyyffyyyyyyyyee; 1^{st}: efyyyfyyyyyyyfyyyyyyyee and 2^{nd}. eeyyyyfyfffyyyyyyyyee; 1^{st}: efyyyfyyyyyyyfyyyyyyyyy and 2^{nd}. yyyyyyfyfffyyyyyyyyyy; 1′: efyyyfyyyyyyyfyyyyyyyee and 2^{nd}. yyyyyyfyfffyyyyyyyyyy; 1^{st}: efyyyfyyyyyyyfyyyyyyyyy and 2^{nd}. eeyyyyfyfffyyyyyyyyee; 1^{st}: efyyyfyyyyyyyfyyyyyyyee and 2^{nd}. eeyyyyyyyffyyyyyyyyee; 1^{st}: efyyyfyyyyyyyfyyyyyyyyy and 2^{nd} yYy)yyyffyyyyyyyyyy; 1^{st}: efyyyfyyyyyyyfyyyyyyyee and 2^{nd}: yyyyyyfyfffyyyyyyyyyy; and 1^{st}: efyyyfyyyyyyyfyyyyyyyyy and 2^{nd}. eeyyyyfyfffyyyyyyyyee, wherein each “y” represents a 2′-OMe sugar moiety, each “e” represents a 2′-MOE sugar moiety, and each “f” represents a 2′-fluoro sugar moiety.

Embodiment A171. The oligomeric duplex of any one of embodiments A119-A170, wherein the internucleoside linkages between the first and second nucleosides and the second and third nucleosides counting from the 5′ end of the first modified oligonucleotide, and the internucleoside linkages between the first and second nucleosides and the second and third nucleosides counting from the 3′ end of the first modified oligonucleotide are phosphorothioate internucleoside linkages and wherein all other internucleoside linkages of the first modified oligonucleotide are phosphodiester internucleoside linkages.

Embodiment A172. The oligomeric duplex of any one of embodiments A119-A171, wherein the internucleoside linkages between the first and second nucleosides and the second and third nucleosides counting from the 5′ end of the second modified oligonucleotide, and the internucleoside linkages between the first and second nucleosides and the second and third nucleosides counting from the 3′ end of the second modified oligonucleotide are phosphorothioate internucleoside linkages and wherein all other internucleoside linkages of the second modified oligonucleotide are phosphodiester internucleoside linkages.

Embodiment A173. The oligomeric duplex of any one of embodiments A119-A172, wherein the nucleobase sequence of the first modified oligonucleotide comprises or consists of the nucleobase sequence of any one of SEQ ID NOs: 967, 968, 971, 972, 974, 975, 1254, 1255, and 1033-1038.

Embodiment A174. The oligomeric duplex of any one of embodiments A1-171, wherein a cytosine nucleobase in the first and/or second modified oligonucleotide is optionally 5-methylcytosine.

Embodiment A175. The oligomeric duplex of any one of embodiments A119-A174, wherein the duplex comprises a conjugate group comprising a conjugate moiety and a conjugate linker.

Embodiment A176. The oligomeric duplex of embodiment A175, wherein the conjugate moiety comprises an active drug substance, an aliphatic chain, a lipid, a peptide, a protein, a hydrocarbon, a polyamine, a polyamide, a polyether, a thioether, an aptamer, an antibody or antibody fragment, a vitamin, a fatty acid, a carbohydrate, an intercalator or a reporter molecule.

Embodiment A177. The oligomeric duplex of embodiment A175, wherein the conjugate group comprises a C22 alkyl, C20 alkyl, C17 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, or C5 alkyl, wherein the alkyl chain optionally has one or more unsaturated bonds.

Embodiment A178. The oligomeric duplex of embodiment A175, wherein the conjugate group comprises a 6-palmitamidohexyl moiety or a 2-(hydroxymethyl)-6-palmitamidohexyl moiety.

Embodiment A179. The oligomeric duplex of embodiment A175, wherein the duplex comprises a conjugate moiety that binds type 1 transferrin receptor (TfR1).

Embodiment A180. The oligomeric duplex of embodiment A179, wherein the conjugate moiety is selected from antibody or fragment thereof, a protein or peptide or an aptamer capable of binding TfR1.

Embodiment A181. The oligomeric duplex of embodiment A180, wherein the conjugate moiety is a cyclic protein or cyclic peptide.

Embodiment A182. The oligomeric duplex of embodiment A181, wherein the conjugate group consists of the bicycle ligand and a conjugate linker.

Embodiment A183. The oligomeric duplex of embodiment A182, wherein the bicycle ligand comprises a polypeptide consisting of 13-22 linked amino acids or amino acid mimetics and a molecular scaffold, wherein each of a first, a second, and a third amino acid of the polypeptide comprises a reactive group, each of which separately forms a bond with the molecular scaffold, thereby forming two polypeptide loops attached to the molecular scaffold.

Embodiment A184. The oligomeric duplex of embodiment A183 wherein the bicycle ligand comprises a sequence selected from any one of SEQ ID NO: 1071-1252.

Embodiment A185. The oligomeric duplex of any one of embodiments A182-A184, wherein the conjugate linker comprises a (bicyclo[6.1.0]nonyne)-formyl (BCN) moiety.

Embodiment A186. The oligomeric duplex of embodiment A185, wherein the conjugate linker comprises a 6-(BCN-carbamate)-2-(hydroxymethyl)hexyl phosphoryl moiety or a 6-(BCN-carbamate)hexyl phosphoryl moiety.

Embodiment A187. The oligomeric duplex of any one of embodiments A119-A174, comprising a conjugate group consisting of a conjugate moiety and a conjugate linker wherein the linker comprises a 6-(BCN-carbamate)-2-(hydroxymethyl)hexyl phosphoryl moiety or a 6-(BCN-carbamate)hexyl phosphoryl moiety.

Embodiment A188. The oligomeric duplex of any one of embodiments A119-A174, comprising a bicycle compound comprising a peptide having the following chemical notation:

(SEQ ID NO: 1045)
Ac-CP[HyP]DAYLGC[tBuGly]SYCEPW[K(N₃)]-NH₂,

wherein:
Ac=acetyl,
C=cysteine,
P=proline,
D=aspartate,
A=alanine,
Y=tyrosine,
L=leucine,
G=glycine,
S=serine,
E=glutamate,
W=tryptophan,
[HyP]=trans-4-hydroxy-L-proline,
[tBuGly]=t-butyl-glycine, and
[K(N₃)]=6-azido lysine; and
wherein each cysteine forms a covalent bond with the molecular scaffold 1,1′,1″-(1,3,5-triazinane-1, 3,5-triyl)tris(2-bromoethanone) (TATB).

Embodiment A189. The oligomeric duplex of any one of embodiments A119-A174, comprising a bicycle compound having the following structure:

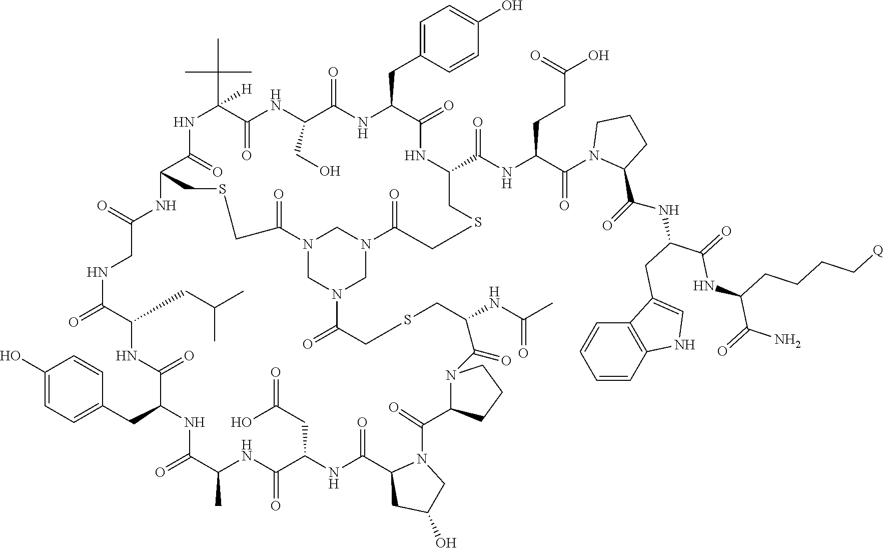

or a salt thereof, wherein Q is N₃ (BCY17901, SEQ ID NO: 1045), NH₂ (BCY21757, SEQ ID NO: 1202), SH (BCY21758, SEQ ID NO:1203), a conjugate linker, or a conjugate linker covalently connected to an oligonucleotide.

Embodiment A190. The oligomeric duplex of embodiment A188, wherein Q is N₃.

Embodiment A191. The oligomeric duplex of any one of embodiments A119-A174, comprising a conjugate group having the following structure:

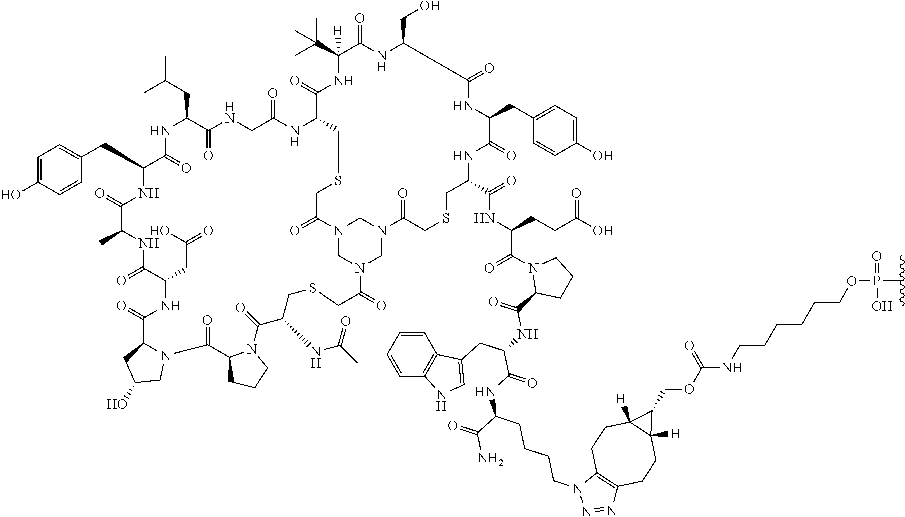

Embodiment A192. The oligomeric duplex of any one of embodiments A119-A174, comprising a conjugate group having the following structure:

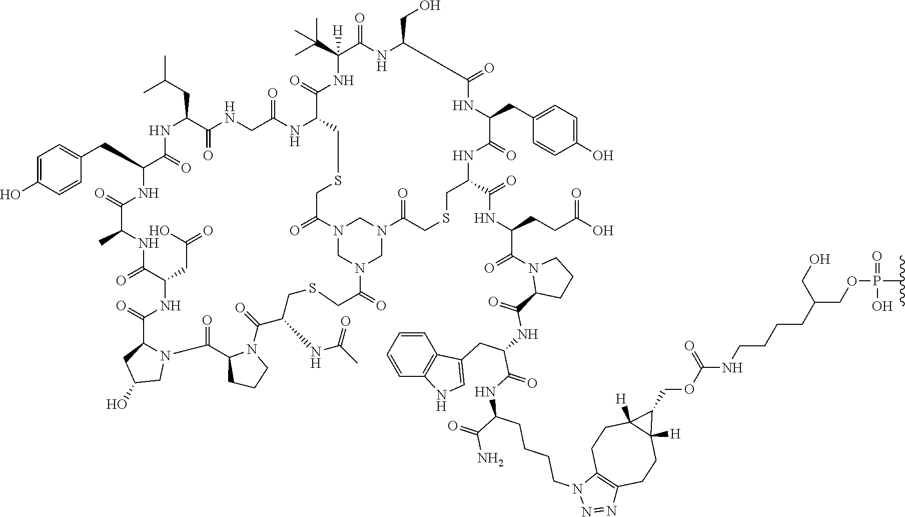

Embodiment A193. The oligomeric duplex of any one of embodiments A175-A191, wherein the bicycle compound or conjugate group is attached to the second modified oligonucleotide.

Embodiment A194. The oligomeric duplex of any one of embodiments A175-A191, wherein the bicycle compound or conjugate group is attached to the 5′- or 3′-end of the second modified oligonucleotide.

Embodiment A195. The oligomeric duplex of any one of embodiments A175-191, wherein the bicycle compound or conjugate group is attached to the 5′-terminal nucleoside of the second modified oligonucleotide.

Embodiment A196. An oligomeric compound comprising a modified oligonucleotide according to the following chemical notation:

(SEQ ID NO: 1033)
vPTₑₛU_{fs}U_{yo}A_{yo}A_{yo}G_{do}A_{yo}U_{yo}G_{yo}A_{yo}G_{yo}A_{yo}C_{yo}A_{fo}G_{yo}A_{fo}
A_{yo}A_{yo}U_{yo}U_{yo}G_{ys}AₑₛAₑ,

wherein:
an adenine nucleobase,
C=a cytosine nucleobase,
G=a guanine nucleobase,
T=a thymine nucleobase,
U=a uracil nucleobase,
d=a 2′-β-D-deoxyribosyl sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate moiety.

Embodiment A197. An oligomeric compound comprising a modified oligonucleotide according to the following chemical notation:

(SEQ ID NO: 1027)
^{m}CₑₛAₑₛA_{yo}U_{yo}U_{yo}U_{yo}C_{yo}U_{yo}G_{yo}U_{fo}C_{fo}U_{yo}C_{yo}A_{yo}U_{yo}C_{yo}
U_{yo}U_{yo}A_{ys}AₑₛAₑ,

wherein:
A=an adenine nucleobase,
^{m}C=a 5-methyl cytosine nucleobase,
C=a cytosine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

Embodiment A198. An oligomeric compound comprising a conjugated modified oligonucleotide according to the following chemical notation: [Ac-C P [HyP]D A Y L G C [tBuGly] S Y C E P W [K(N₃)]—NH₂]-[6-(BCN-carbamate)hexyl]ₒ^{m}CₑₛAₑₛA_{yo}U_{yo}U_{yo}U_{yo}C_{yo}U_{yo}G_{yo}U_{yo}C_{yo}U_{yo}C_{yo}A_{yo}U_{yo}C_{yo}U_{yo}U_{yo}A_{ys}AₑₛAₑ (SEQ ID NO: 1039), wherein:
Ac=acetyl, C=cysteine, [HyP]=trans-4-hydroxy-L-proline, D=aspartate, A=alanine, Y=tyrosine, L=leucine, G=glycine, [tBuGly]=t-butyl-glycine, S=serine, E=glutamate, P=proline, W=tryptophan, [K(N)]=6-azido lysine, NH₂=amino, BCN=(bicyclo[6.1.0]nonyne)-formyl, “^{m}C=a 5-methyl cytosine nucleobase, C=a cytosine nucleobase, A=an adenine nucleobase, G=a guanine nucleobase, U=a uracil nucleobase, e=a 2′-MOE sugar moiety, f=a 2′-fluoro sugar moiety, y=a 2′-OMe sugar moiety, o=a phosphodiester internucleoside linkage, and s=a phosphorothioate internucleoside linkage;
and wherein each cysteine forms a covalent bond with the molecular scaffold 1,1′,1″-(1,3,5-triazinane-1,3,5-triyl)tris(2-bromoethanone) (TATB).

Embodiment A199. An oligomeric duplex comprising the oligomeric compound according to embodiment A195 and the oligomeric compound according to embodiment A196.

Embodiment A200. An oligomeric duplex comprising the oligomeric compound according to embodiment A195 and the oligomeric compound according to embodiment A197.

Embodiment A201. An oligomeric compound comprising a modified oligonucleotide according to the following chemical notation: vP-TₑₛU_{fs}U_{yo}A_{yo}A_{do}G_{yo}A_{do}U_{yo}G_{yo}A_{yo}G_{yo}A_{yo}C_{yo}A_{fo}G_{yo}A_{fo}A_{yo}A_{yo}U_{yo}U_{yo}G_{ys}AₑₛAₑ (SEQ ID NO: 1034), wherein:
A=an adenine nucleobase,
C=a cytosine nucleobase,
G=a guanine nucleobase,
T=a thymine nucleobase,
U=a uracil nucleobase,
d=a 2′-β-D-deoxyribosyl sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate moiety.

Embodiment A202. An oligomeric compound comprising a modified oligonucleotide according to the following chemical notation: CₑₛAₑₛA_{yo}U_{yo}U_{yo}U_{yo}C_{fo}U_{yo}G_{fo}U_{yo}C_{fo}U_{yo}C_{yo}A_{yo}U_{yo}C_{yo}U_{yo}U_{yo}A_{ys}AₑₛAₑ (SEQ ID NO: 1028), wherein:
A=an adenine nucleobase,
C=a cytosine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

Embodiment A203. An oligomeric compound comprising a conjugated modified oligonucleotide according to the following chemical notation: [Ac—C P [HvP] D A Y L G C [tBuGly] S Y C E P W [K(N₃]—NH₂]-[6-(BCN-carbamate)hexyl]ₒCₑₛAₑₛA_{yo}U_{yo}U_{yo}U_{yo}C_{yo}U_{yo}G_{yo}U_{yo}C_{yo}U_{yo}C_{yo}A_{yo}U_{yo}C_{yo}U_{yo}U_{yo}A_{ys}AₑₛAₑ (SEQ ID NO: 1040), wherein:
Ac=acetyl, C=cysteine, [HyP]=trans-4-hydroxy-L-proline, D=aspartate, A=alanine, Y=tyrosine, L=leucine, G=glycine, [tBuGly]=t-butyl-glycine, S=serine, E=glutamate, P=proline, W=tryptophan, [K(N₃)]=6-azido lysine, NH₂=amino, BCN=(bicyclo[6.1.0]nonyne)-formyl, C=a cytosine nucleobase, A=an adenine nucleobase, G=a guanine nucleobase, U=a uracil nucleobase, e=a 2′-MOE sugar moiety, f=a 2′-fluoro sugar moiety, y=a 2′-OMe sugar moiety, o=a phosphodiester internucleoside linkage, and s=a phosphorothioate internucleoside linkage;
   and wherein each cysteine of the peptide forms a covalent bond with a molecular scaffold of 1,1′,1″-(1,3,5-triazinane-1,3,5-triyl)tris(2-bromoethanone) (TATB).

Embodiment A204. An oligomeric duplex comprising an oligomeric compound of embodiment A200 and an oligomeric compound of embodiment A201.

Embodiment A205. An oligomeric duplex comprising an oligomeric compound of embodiment A200 and an oligomeric compound of embodiment A202.

Embodiment A206. An oligomeric compound comprising a modified oligonucleotide according to the following chemical notation:

(SEQ ID NO: 1035)
vPTₑₛA_{fs}U_{yo}A_{yo}A_{yo}A_{do}U_{yo}A_{yo}G_{yo}A_{yo}U_{yo}U_{yo}C_{yo}U_{fo}G_{yo}U_{fo}
A_{yo}G_{yo}C_{yo}U_{yo}U_{ys}AₑₛAₑ,

wherein:
A=an adenine nucleobase,
C=a cytosine nucleobase,
G=a guanine nucleobase,
T=a thymine nucleobase,
U=a uracil nucleobase,
d=a 2′-β-D-deoxyribosyl sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate moiety.

Embodiment A207. An oligomeric compound comprising a modified oligonucleotide according to the following chemical notation: AₑₛAₑₛG_{yo}C_{yo}U_{yo}A_{yo}C_{yo}A_{yo}G_{yo}A_{fo}A_{fo}U_{yo}C_{yo}U_{yo}A_{yo}U_{yo}U_{yo}U_{yo}A_{ys}TₑₛAₑ (SEQ ID NO: 1029), wherein:
A=an adenine nucleobase,
C=a cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

Embodiment A208. An oligomeric compound comprising a conjugated modified oligonucleotide according to the following chemical notation: [Ac—C P [HyP] D A Y L C [tBuGly] S Y C E P W [K(N₃)]—NH₂]-[6-(BCN-carbamate)hexyl]ₒAₑₛAₑₛG_{yo}C_{yo}U_{yo}A_{yo}C_{yo}A_{yo}G_{yo}A_{yo}A_{yo}U_{yo}C_{yo}U_{yo}A_{yo}U_{yo}U_{yo}U_{yo}A_{ys}TₑₛAₑ (SEQ ID NO: 1041), wherein:
Ac=acetyl, C=cysteine, [HyP]=trans-4-hydroxy-L-proline, D=aspartate, A=alanine, Y=tyrosine, L=leucine, G=glycine, [tBuGly]=t-butyl-glycine, S=serine, E=glutamate, P=proline, W=tryptophan, [K(N₃]=6-azido lysine, NH₂=amino, BCN=(bicyclo[6.1.0]nonyne)-formyl, C=a cytosine nucleobase, A=an adenine nucleobase, G=a guanine nucleobase, T=a thymine nucleobase, U=a uracil nucleobase, e=a 2′-MOE sugar moiety, f=a 2′-fluoro sugar moiety, y=a 2′-OMe sugar moiety, o=a phosphodiester internucleoside linkage, and s=a phosphorothioate internucleoside linkage;
   and wherein each cysteine of the peptide forms a covalent bond with a molecular scaffold of 1,1′,1″-(1,3,5-triazinane-1,3,5-triyl)tris(2-bromoethanone) (TATB).

Embodiment A209. An oligomeric duplex comprising an oligomeric compound of embodiment A205 and an oligomeric compound of embodiment A206.

Embodiment A210. An oligomeric compound comprising an oligomeric compound of embodiment A205 and an oligomeric compound of embodiment A207.

Embodiment A211. An oligomeric compound comprising a modified oligonucleotide according to the following chemical notation:

(SEQ ID NO: 1036)
vPTₑₛA_{fs}U_{yo}A_{yo}A_{do}A_{yo}T_{do}A_{yo}G_{yo}A_{yo}U_{yo}U_{yo}C_{yo}U_{fo}G_{yo}U_{fo}
A_{yo}G_{yo}C_{yo}U_{yo}U_{ys}AₑₛAₑ,

wherein:
A=an adenine nucleobase,
C=a cytosine nucleobase,
G=a guanine nucleobase,
T=a thymine nucleobase,
U=a uracil nucleobase,
d=a 2′-β-D-deoxyribosyl sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate moiety.

Embodiment A212. An oligomeric compound comprising a modified oligonucleotide according to the following chemical notation: AₑₛAₑₛG_{yo}C_{yo}U_{yo}A_{yo}C_{fo}A_{yo}G_{fo}A_{yo}A_{fo}U_{yo}C_{yo}U_{yo}A_{yo}U_{yo}U_{yo}U_{yo}A_{ys}UₑₛAₑ (SEQ ID NO: 1030), wherein:
A=an adenine nucleobase,
C=a cytosine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

Embodiment A213. An oligomeric compound comprising a conjugated modified oligonucleotide conjugate according to the following chemical notation: [Ac—C P [HyP] D A X L G C [tBuGly] S Y C E P W [K(N₃]—NH₂]-[6-(BCN-carbamate)hexyl]ₒAₑₛAₑₛG_{yo}C_{yo}U_{yo}A_{yo}C_{fo}A_{yo}G_{fo}A_{yo}A_{fo}U_{yo}C_{yo}U_{yo}A_{yo}U_{yo}U_{yo}U_{yo}A_{ys}UₑₛAₑ (SEQ ID NO: 1042), wherein:
Ac=acetyl, C=cysteine, [HyP]=trans-4-hydroxy-L-proline, D=aspartate, A=alanine, Y=tyrosine, L=leucine, G=glycine, [tBuGly]=t-butyl-glycine, S=serine, E=glutamate, P=proline, W=tryptophan, [K(N₃]=6-azido lysine, NH₂=amino, BCN=(bicyclo[6.1.0]nonyne)-formyl, C=a cytosine nucleobase, A=an adenine nucleobase, G=a guanine nucleobase, U=a uracil nucleobase, e=a 2′-MOE sugar moiety, f=a 2′-fluoro sugar moiety, y=a 2′-OMe sugar moiety, o=a phosphodiester internucleoside linkage, and s=a phosphorothioate internucleoside linkage;
   and wherein each cysteine of the peptide forms a covalent bond with a molecular scaffold of 1,1′,1″-(1,3,5-triazinane-1,3,5-triyl)tris(2-bromoethanone) (TATB).

Embodiment A214. An oligomeric duplex comprising an oligomeric compound of embodiment A210 and an oligomeric compound of embodiment A211.

Embodiment A215. An oligomeric duplex comprising an oligomeric compound of embodiment A210 and an oligomeric compound of embodiment A212.

Embodiment A216. An oligomeric compound comprising a modified oligonucleotide according to the following chemical notation:

(SEQ ID NO: 1037)
vPTₑₛU_{fs}A_{yo}A_{yo}G_{yo}T_{do}U_{yo}U_{yo}U_{yo}A_{yo}G_{yo}U_{yo}C_{yo}U_{fo}U_{yo}A_{fo}
A_{yo}U_{yo}C_{yo}U_{yo}U_{ys}AₑₛAₑ,

wherein:
A=an adenine nucleobase,
C=a cytosine nucleobase,
G=a guanine nucleobase,
T=a thymine nucleobase,
U=a uracil nucleobase,
d=a 2′-β-D-deoxyribosyl sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate moiety.

Embodiment A217. An oligomeric compound comprising a modified oligonucleotide according to the following chemical notation: AₑₛAₑₛG_{yo}A_{yo}U_{yo}U_{yo}A_{yo}A_{yo}G_{yo}A_{fo}C_{fo}U_{yo}A_{yo}A_{yo}A_{yo}A_{yo}C_{yo}U_{yo}U_{ys}AₑₛAₑ (SEQ ID NO: 1031), wherein:
A=an adenine nucleobase,
C=a cytosine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

Embodiment A218. An oligomeric compound comprising a conjugated modified oligonucleotide according to the following chemical notation: [Ac—C P [HyP] D A Y L G C [tBuGly] S Y C E P W [K(N₃]—NH₂]-[6-(BCN-carbamate)hexyl]ₒAₑₛAₑₛG_{yo}A_{yo}U_{yo}U_{yo}A_{yo}A_{yo}G_{yo}A_{yo}C_{yo}U_{yo}A_{yo}A_{yo}A_{yo}A_{yo}C_{yo}U_{yo}U_{ys}AₑₛAₑ (SEQ ID NO: 1043), wherein:
Ac=acetyl, C=cysteine, [HyP]=trans-4-hydroxy-L-proline, D=aspartate, A=alanine, Y=tyrosine, L=leucine, G=glycine, [tBuGly]=t-butyl-glycine, S=serine, E=glutamate, P=proline, W=tryptophan, [K(N₃]=6-azido lysine, NH₂=amino, BCN=(bicyclo[6.1.0]nonyne)-formyl, C=a cytosine nucleobase, A=an adenine nucleobase, G=a guanine nucleobase, U=a uracil nucleobase, e=a 2′-MOE sugar moiety, f=a 2′-fluoro sugar moiety, y=a 2′-OMe sugar moiety, o=a phosphodiester internucleoside linkage, and s=a phosphorothioate internucleoside linkage;
   and wherein each cysteine of the peptide forms a covalent bond with a molecular scaffold of 1,1′,1″-(1,3,5-triazinane-1,3,5-triyl)tris(2-bromoethanone) (TATB).

Embodiment A219. An oligomeric duplex comprising the modified oligonucleotide according to embodiment A215 and the modified oligonucleotide according to embodiment A216.

Embodiment A220. An oligomeric duplex comprising the modified oligonucleotide according to embodiment A215 and the conjugate according to embodiment A217.

Embodiment A221. An oligomeric compound comprising a modified oligonucleotide according to the following chemical notation

(SEQ ID NO: 1038)
vPTₑₛU_{fs}A_{yo}A_{yo}G_{do}U_{yo}T_{do}U_{yo}U_{yo}A_{yo}G_{yo}U_{yo}C_{yo}U_{fo}U_{yo}A_{fo}
A_{yo}U_{yo}C_{yo}U_{yo}U_{ys}AₑₛAₑ,

wherein:
A=an adenine nucleobase,
C=a cytosine nucleobase,
G=a guanine nucleobase,
T=a thymine nucleobase,
U=a uracil nucleobase,
d=a 2′-β-D-deoxyribosyl sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate moiety.

Embodiment A222. An oligomeric compound comprising a modified oligonucleotide according to the following chemical notation: AₑₛAₑₛG_{yo}A_{yo}U_{yo}U_{yo}A_{fo}A_{yo}G_{fo}A_{yo}C_{fo}U_{yo}A_{yo}A_{yo}A_{yo}A_{yo}C_{yo}U_{yo}U_{ys}AₑₛAₑ (SEQ ID NO: 1032), wherein:
A=an adenine nucleobase,
C=a cytosine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

Embodiment A223. An oligomeric compound comprising a conjugated modified oligonucleotide according to the following chemical notation: [Ac—C P [HyP] D A Y L C [tBuGly] S Y C E P W [K(N₃)]—NH₂]-[6-(BCN-carbamate)hexyl]ₒAₑₛAₑₛG_{yo}A_{yo}U_{yo}U_{yo}A_{fo}A_{yo}G_{fo}A_{yo}C_{fo}U_{yo}A_{yo}A_{yo}A_{yo}A_{yo}C_{yo}U_{yo}U_{ys}AₑₛAₑ (SEQ ID NO: 1044), wherein:
Ac=acetyl, C=cysteine, [HyP]=trans-4-hydroxy-L-proline, D=aspartate, A=alanine, Y=tyrosine, L=leucine, G=glycine, [tBuGly]=t-butyl-glycine, S=serine, E=glutamate, P=proline, W=tryptophan, [K(N₃)]=6-azido lysine, NH₂=amino, BCN=(bicyclo[6.1.0]nonyne)-formyl, C=a cytosine nucleobase, A=an adenine nucleobase, G=a guanine nucleobase, U=a uracil nucleobase, e=a 2′-MOE sugar moiety, f=a 2′-fluoro sugar moiety, y=a 2′-OMe sugar moiety, o=a phosphodiester internucleoside linkage, and s=a phosphorothioate internucleoside linkage;
   and wherein each cysteine of the peptide forms a covalent bond with a molecular scaffold of 1,1′,1″-(1,3,5-triazinane-1,3,5-triyl)tris(2-bromoethanone) (TATB).

Embodiment A224. An oligomeric duplex comprising an oligomeric compound of embodiment A220 and an oligomeric compound of embodiment A221.

Embodiment A225. An oligomeric duplex comprising an oligomeric compound of embodiment A220 and an oligomeric compound of embodiment A222.

A268. A population of oligomeric duplexed or oligomeric compounds of any of embodiments A1-A225, wherein the population is enriched for first and/or second modified oligonucleotides comprising at least one particular phosphorothioate internucleoside linkage having a particular stereochemical configuration.

Embodiment A269. The population of embodiment A267, wherein the population is enriched for first and/or second modified oligonucleotides comprising at least one particular phosphorothioate internucleoside linkage having the (Sp) or (Rp) configuration.

Embodiment A270. The oligomeric duplex or oligomeric compound of any one of embodiments A1-A225, wherein the first modified oligonucleotide consists of 23 linked nucleosides and the second modified oligonucleotide consists of 21 linked nucleosides.

Embodiment A271. An antisense compound comprising or consisting of an oligomeric duplex or oligomeric compound of any one of embodiments A1-A225.

Embodiment A272. The antisense compound of embodiment A270, wherein the antisense compound is an RNAi agent capable of reducing the amount of PLN nucleic acid through the activation of RISC/Ago2.

Embodiment A273. A pharmaceutical composition comprising the oligomeric duplex or oligomeric compound of any one of embodiments A1-A225 or A269, the population of any one of embodiments A267-A268, or the antisense compound of embodiment A270 or embodiment A271, and a pharmaceutically acceptable diluent or carrier.

Embodiment A274. The pharmaceutical composition of embodiment A272, wherein the pharmaceutically acceptable diluent is water or phosphate-buffered saline.

Embodiment A275. The pharmaceutical composition of embodiment A273, wherein the pharmaceutical composition consists essentially of the oligomeric duplex, oligomeric compound or the antisense compound, and water or phosphate-buffered saline.

Embodiment A276. A method of decreasing the amount of PLN RNA or PLN protein in a cell, tissue, organ or subject, comprising contacting the cell, tissue, organ or subject with the oligomeric duplex or oligomeric compound of any one of embodiments A1-A225 or A269, the population of any one of embodiments A267-A268, the antisense compound of embodiment A270 or embodiment A271, or the pharmaceutical composition of any one of embodiments A272-A274.

Embodiment A277. The method of embodiment A275, wherein the cell is a muscle cell and/or a cardiac cell.

Embodiment A278. A method comprising administering to a subject the oligomeric duplex or oligomeric compound of any one of embodiments A1-A225 or A269, the population of any one of embodiments A267-A268, the antisense compound of embodiment A270 or embodiment A271, or the pharmaceutical composition of any one of embodiments A272-A274; wherein the subject has or is at risk for developing a cardiovascular or cardiac injury, disease, condition or disorder, cardiomyopathy, cardiac arrythmia, and/or heart failure.

Embodiment A279. A method of preventing or treating a disease, disorder, condition or injury associated with cardiac calcium misregulation, or postponing a symptom of a disease, disorder or condition associated with a damaged, weakened and/or overworked heart, comprising administering to a subject having, or at risk of having, a disease, disorder or condition associated with cardiac calcium misregulation a therapeutically effective amount of the oligomeric duplex or oligomeric compound of any one of embodiments A1-A225 or A269, the population of any one of embodiments A267-A268, the antisense compound of embodiment A270 or embodiment A271, or the pharmaceutical composition of any one of embodiments A272-A274; wherein the disease, disorder, condition or injury is selected from a cardiac or cardiovascular disease, disorder, condition or injury, a cardiomyopathy, cardiac arrythmia, and/or heart failure.

Embodiment A280. A method of preventing or treating a cardiac or cardiovascular disease, disorder, condition or injury associated with heart failure, or postponing a symptom of heart failure, comprising administering to a subject having, or at risk of having, a cardiac or cardiovascular disease, disorder, condition or injury a therapeutically effective amount of the oligomeric duplex or oligomeric compound of any one of embodiments A1-A225 or A269, the population of any one of embodiments A267-A268, the antisense compound of embodiment A270 or embodiment A271, or the pharmaceutical composition of any one of embodiments A272-A274; wherein the disease, disorder, condition or injury is a cardiomyopathy, cardiac arrythmia, and/or heart failure.

Embodiment A281. A method of decreasing the amount of PLN RNA and/or PLN protein in the heart of a subject having or at risk of developing a disease, disorder or condition associated with a damaged, weakened and/or overworked heart, comprising administering to a subject having, or at risk of having, a disease, disorder or condition associated with a damaged, weakened and/or overworked heart a therapeutically effective amount of the oligomeric duplex or oligomeric compound of any one of embodiments A1-A225 or A269, the population of any one of embodiments A267-A268, the antisense compound of embodiment A270 or embodiment A271, or the pharmaceutical composition of any one of embodiments A272-A274; wherein the disease, disorder, condition or injury is a cardiomyopathy, cardiac arrythmia, and/or heart failure.

Embodiment A282. The method of any one of embodiments A275-A280, wherein the amount of PLN RNA and/or PLN protein in cardiac muscle of the subject is decreased.

Embodiment A283. The method of any one of embodiments A276-A281, wherein the disease, disorder or condition is cardiomyopathy or arrhythmia.

Embodiment A284. The method of embodiment A282, wherein the cardiomyopathy is genetic cardiomyopathy.

Embodiment A285. The method of embodiment A283, wherein the genetic cardiomyopathy is associated with p.Arg14del, Arg9Cys (R9C), or Arg25Cys (R25C) genetic mutations.

Embodiment A286. The method of embodiment A282, wherein the cardiomyopathy is dilated cardiomyopathy (DCM).

Embodiment A287. The method of embodiment A285 wherein the DCM is genetic DCM.

Embodiment A288. The method of embodiment A286, wherein the genetic DCM is associated with TTN, LMNA, RBM20, SCN5A, MYH7, TNNT2, and TPM1 mutations.

Embodiment A289. The method of embodiment A285, wherein the DCM is arrhythmogenic DCM.

Embodiment A290. The method of any one of embodiments A277-A281, wherein the heart failure is heart failure with preserved ejection fraction (HFpEF), heart failure with reduced ejection fraction (HFrEF), acute heart failure, or worsening of chronic heart failure.

Embodiment A291. The method of embodiment A282, wherein the arrhythmia is ventricular tachycardia (vtac) or ventricular fibrillation (vfib).

Embodiment A292. The method of embodiment A278, wherein the symptom of a disease, disorder or condition associated with a damaged, weakened and/or overworked heart, or heart failure is reduced myocardial contractile function and/or impaired relaxation of the heart.

Embodiment A293. The method of any one or embodiments A277A-291, wherein the method prevents or slows progression of damage, weakening and/or overworked heart effects and/or heart failure.

Embodiment A294. The method of any one of embodiments A277-A291, wherein administering of the oligomeric duplex or oligomeric compound of any one of embodiments A1-A225 or A269, the population of any one of embodiments A267-A268, the antisense compound of embodiment A270 or embodiment A271, or the pharmaceutical composition of any one of embodiments A272-A274 improves cardiac function, cardiovascular death, cardiac dilation, cardiac fibrosis, low voltage ECG, diastolic calcium uptake, ejection fraction (EF), left ventricular ejection fraction (LVEF), left ventricular end systolic volume (LVESV), left ventricular end diastolic volume (LVEDV), mitral valve flow profile, left ventricle (LV) strain, left ventricle (LV) strain rate, infarct size, heart failure hospitalization, 6 minute walk test (6MWT), the Kansas City Cardiomyopathy Questionnaire Score (KCCQS), heart rate, or heart rhythm in the subject.

Embodiment A295. The method of any one of embodiments A275-A293, wherein the oligomeric duplex or oligomeric compound of any one of embodiments A1-A225 or A269, the population of any one of embodiments A267-A268, the antisense compound of embodiment A270 or embodiment A271, or the pharmaceutical composition of any one of embodiments A272-A274 comprises a conjugate moiety having affinity for a transferrin receptor.

Embodiment A296. The method any one of embodiments A275-A293, wherein the oligomeric duplex or oligomeric compound of any one of embodiments A1-A225 or A269, the population of any one of embodiments A267-A268, the antisense compound of embodiment A270 or embodiment A271, or the pharmaceutical composition of any one of embodiments A272-A274 comprises a bicycle compound having affinity for a transferrin receptor.

Embodiment A297. Use of the oligomeric duplex or oligomeric compound of any one of embodiments A1-A225 or A269, the population of any one of embodiments A267-268, the antisense compound of embodiment A270 or embodiment A271, or the pharmaceutical composition of any one of embodiments A272-A274 for treating or preventing a disease, disorder or condition associated with a damaged, weakened and/or overworked heart or heart failure.

Embodiment A298. Use of the oligomeric duplex or oligomeric compound of any one of embodiments A1-A225 or A269, the population of any one of embodiments A267-A268, the antisense compound of embodiment A270 or embodiment A271, or the pharmaceutical composition of any one of embodiments A272-A274 in the manufacture of a medicament for treating or preventing a disease, disorder or condition associated with a damaged, weakened and/or overworked heart or heart failure.

Embodiment A299. The use of embodiment A296 or A297, wherein the disease, disorder or condition associated with a damaged, weakened and/or overworked heart or associated with heart failure is cardiomyopathy, cardiac arrythmia, and/or heart failure.

Embodiment A300. The use of embodiment A298, wherein the cardiomyopathy is genetic cardiomyopathy.

Embodiment A301. The use of embodiment A299, wherein the genetic cardiomyopathy is associated with p.Arg14del, Arg9Cys (R9C), or Arg25Cys (R25C) genetic mutations.

Embodiment A302. The use of embodiment A298, wherein the cardiomyopathy is dilated cardiomyopathy (DCM).

Embodiment A303. The use of embodiment A301, wherein the DCM is genetic DCM.

Embodiment A304. The use of embodiment A302, wherein the genetic DCM is associated with TTN, LMNA, RBM20, SCN5A, MYH7, TNNT2, and TPM1 mutations.

Embodiment A305. The use of embodiment A301, wherein the DCM is arrhythmogenic DCM.

Embodiment A306. The use of embodiment A298, wherein the heart failure is heart failure with preserved ejection fraction (HFpEF), heart failure with reduced ejection fraction (HFrEF), acute heart failure, or worsening of chronic heart failure.

Embodiment A307. The use of embodiment A298, wherein the arrhythmia is ventricular tachycardia (vtac) or ventricular fibrillation (vfib).

Embodiment B1. An oligomeric duplex comprising a first oligomeric compound and a second oligomeric compound, wherein:
(1) a first oligomeric compound comprises a modified oligonucleotide consisting of 8 to 50 linked nucleosides, wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases of the nucleobase sequence of any one of SEQ ID NOs: 3-314, 627-782, 939-976, 1033-1038, or 1254-1255 wherein each of the nucleosides of the first modified oligonucleotide independently comprises a modified sugar moiety or sugar surrogate and wherein at least one modified nucleoside and less than 40% of the nucleosides of the first modified oligonucleotide comprises a fluorine; and
(2) a second oligomeric compound comprises a modified oligonucleotide consisting of 8 to 50 contiguous linked nucleosides wherein the nucleobase sequence of the second modified oligonucleotide comprises at least 8, at least 9, at least 10, at least 11, at least 12, at least 13, at least 14, at least 15, at least 16, at least 17, at least 18, at least 19, at least 20, or at least 21 contiguous nucleobases of the nucleobase sequence of any one of SEQ ID NOs: 315-626, 783-938, 977-1016 or 1027-1032, wherein each of the nucleosides of the second modified oligonucleotide independently comprises a modified sugar moiety or sugar surrogate and wherein less than 40% of the nucleosides of the second modified oligonucleotide comprises a fluorine.

Embodiment B2. The oligomeric duplex of embodiment B1, wherein the first modified oligonucleotide is at least 80%, at least 85%, at least 90%, at least 95%, or at least 99% complementary to an equal length portion of a PLN nucleic acid, wherein the PLN nucleic acid has the nucleobase sequence of SEQ ID NO: 1 or SEQ ID NO: 2.

Embodiment B3. The oligomeric duplex of embodiment B1 or embodiment B2, wherein each of the nucleosides of the first modified oligonucleotide independently and the second modified oligonucleotide independently comprises a modified sugar moiety or sugar surrogate independently selected from a 2′-F sugar moiety, 2′-MOE sugar moiety, 2′-OMe sugar moiety, 2′-deoxyribosyl sugar moiety, and 3′-fluoro-hexitol sugar moiety.

Embodiment B4. The oligomeric duplex of any one of embodiments B1-B3, wherein fewer than 35%, fewer than 30%, fewer than 25%, fewer than 20%, fewer than 15%, fewer than 14%, fewer than 13%, fewer than 12%, fewer than 10%, fewer than 5%, or fewer than 1% of the nucleosides in the first modified oligonucleotide comprises a fluorine.

Embodiment B5. The oligomeric duplex of any one of embodiments B1-B4, wherein no more than 1 nucleoside, no more than 2 nucleosides, no more than 3 nucleosides, or no more than 4 nucleosides in the first modified oligonucleotide comprise a modified sugar moiety comprising a fluorine or a sugar surrogate comprising a fluorine.

Embodiment B6. The oligomeric duplex of any one of embodiments B1-5, wherein no more than 1 nucleoside, no more than 2 nucleosides, or no more than 3 nucleosides within the sequence of the first modified oligonucleotide between and including the third and twenty-first nucleosides counting from the 5′ end of the modified oligonucleotide comprise a modified sugar moiety comprising a fluorine or a sugar surrogate comprising a fluorine.

Embodiment B7. The oligomeric duplex of any one of embodiments B1-6, wherein a nucleoside comprising a modified sugar moiety comprising a fluorine or a sugar surrogate comprising a fluorine of the first modified oligonucleotide is independently selected from:
i. the second nucleoside counting from the 5′ end,
ii. the fourteenth and sixteenth nucleosides counting from the 5′ end, or
iii. the second, fourteenth and sixteenth nucleosides counting from the 5′ end; wherein each modified sugar moiety comprising a fluorine or sugar surrogate comprising a fluorine is independently a 2′-fluoro sugar moiety or a 3′-fluoro-hexitol sugar moiety.

Embodiment B8. The oligomeric duplex of any one of embodiments B1-B7, wherein no more than one of the modified sugar moiety comprising a fluorine in the first modified oligonucleotide is a 3′-fluoro-hexitol sugar moiety.

Embodiment B9. The oligomeric duplex of any one of embodiments B1-B8, wherein one or more nucleosides of the first modified oligonucleotide is a 2′-deoxynucleoside.

Embodiment B10. The oligomeric duplex of embodiment B9, wherein the one or more 2′-deoxynucleosides is one or more nucleosides in a region of the sequence of the first modified oligonucleotide between and including the fifth nucleoside to the sixteenth nucleoside counting from the 5′ end of the first modified oligonucleotide.

Embodiment B11. The oligomeric duplex of embodiment B10, wherein the one or more 2′-deoxynucleosides is in a region of the sequence of the first modified oligonucleotide that is any of the fifth, sixth, and/or seventh nucleosides or that is any of the fourteenth, fifteenth, and/or sixteenth nucleosides counting from the 5′ end of the first modified oligonucleotide.

Embodiment B12. The oligomeric duplex of any one of embodiments B9-B11, wherein fewer than 20%, or fewer than 15%, of the nucleosides of the first modified oligonucleotide comprises a fluorine.

Embodiment B13. The oligomeric duplex of any one of embodiments B9-B12, wherein the one or more 2′-deoxynucleosides is the fifth, sixth and/or seventh nucleoside(s) counting from the 5′ end of the first modified oligonucleotide.

Embodiment B14. The oligomeric duplex of any one of embodiments B9-B13, wherein only two nucleosides of the first modified oligonucleotide are 2′-deoxynucleosides.

Embodiment B15. The oligomeric duplex of embodiment B14, wherein the two 2′-deoxynucleosides are the fifth and seventh nucleosides, or the fourteenth and sixteenth nucleosides, counting from the 5′ end of the first modified oligonucleotide.

Embodiment B16. The oligomeric duplex of embodiment B14, wherein the two 2′-deoxynucleosides are the fifth and seventh nucleosides counting from the 5′ end of the first modified oligonucleotide.

Embodiment B17. The oligomeric duplex of any one of embodiments B9-B13, wherein only one of the nucleosides of the first modified oligonucleotide is a 2′-deoxynucleoside.

Embodiment B18. The oligomeric duplex of embodiment B17, wherein the 2′-deoxynucleoside is the sixth or sixteenth nucleoside counting from the 5′ end of the first modified oligonucleotide.

Embodiment B19. The oligomeric duplex of embodiment B17, wherein the 2′-deoxynucleoside is the sixth nucleoside counting from the 5′ end of the first modified oligonucleotide.

Embodiment B20. The oligomeric duplex of any one of embodiments B1-B19, wherein one or more of the nucleosides of the first modified oligonucleotide independently comprises a sugar surrogate or a bicyclic sugar moiety.

Embodiment B21. The oligomeric duplex of embodiment B20, wherein the bicyclic sugar moiety comprises a 2′-4′ bridge selected from —O—CH₂—; and —O—CH(CH₃)—.

Embodiment B22. The oligomeric duplex of any one of embodiments B1-B21, wherein one or more of the nucleosides of the first modified oligonucleotide comprises a 2′-OMe sugar moiety.

Embodiment B23. The oligomeric duplex of embodiment B22, wherein at least 10%, at least 20%, at least 30%, at least 40%, at least 50% or at least 60% of the nucleosides of the first modified oligonucleotide comprise a 2′-OMe sugar moiety.

Embodiment B24. The oligomeric duplex of embodiment B22 or embodiment B23, wherein the one or more nucleosides comprising a 2′-OMe sugar moiety are in a region of the sequence of the first modified oligonucleotide between and including the third and twenty-first nucleosides counting from the 5′ end of the first modified oligonucleotide.

Embodiment B25. The oligomeric duplex of embodiment B22 or embodiment B23, wherein the 3′-terminal nucleoside of the first modified oligonucleotide comprises a 2′-OMe sugar moiety and/or the nucleoside immediately 5′ of the 3′-terminal nucleoside of the first modified oligonucleotide comprises a 2′-OMe sugar moiety.

Embodiment B26. The oligomeric duplex of any one of embodiments B1-B25, wherein one or more of the nucleosides of the first modified oligonucleotide comprise a 2′-MOE sugar moiety.

Embodiment B27. The oligomeric duplex of embodiment B26, wherein the 5′- and/or 3′-terminal nucleosides of the first modified oligonucleotide comprise a 2′-MOE sugar moiety.

Embodiment B28. The oligomeric duplex of any one of embodiments B1-B27, wherein the nucleoside immediately 5′ of the 3′-terminal nucleoside of the first modified oligonucleotide comprises a 2′-MOE sugar moiety.

Embodiment B29. The oligomeric duplex of any one of embodiments B1-B24 and B26-B28, wherein the 5′- and 3′-terminal nucleosides of the first modified oligonucleotide comprise a 2′-MOE sugar moiety and the nucleoside immediately 5′ of the 3′-terminal nucleoside of the first modified oligonucleotide comprises a 2′-MOE sugar moiety.

Embodiment B30. The oligomeric duplex of any one of embodiments B1-B29, wherein the first modified oligonucleotide comprises a stabilized phosphate group attached to the 5′-terminal nucleoside.

Embodiment B31. The oligomeric duplex of embodiment B30, wherein the stabilized phosphate group comprises a cyclopropyl phosphonate or a vinyl phosphonate.

Embodiment B32. The oligomeric duplex of any one of embodiments B1-B31, wherein the duplex comprises a conjugate group comprising a conjugate moiety and a conjugate linker.

Embodiment B33. The oligomeric duplex of embodiment B32, wherein the conjugate group comprises a cell-targeting moiety.

Embodiment B34. The oligomeric duplex of embodiment B32, wherein the conjugate moiety comprises an active drug substance, an aliphatic chain, a lipid, a peptide, a protein, a hydrocarbon, a polyamine, a polyamide, a polyether, a thioether, an aptamer, an antibody or antibody fragment, a vitamin, a fatty acid, a carbohydrate, an intercalator or a reporter molecule.

Embodiment B35. The oligomeric duplex of embodiment B32, wherein the conjugate group comprises a C22 alkyl, C20 alkyl, C17 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, or C5 alkyl, wherein the alkyl chain optionally has one or more unsaturated bonds.

Embodiment B36. The oligomeric duplex of embodiment B32, wherein the conjugate group comprises a 6-palmitamidohexyl moiety or a 2-(hydroxymethyl)-6-palmitamidohexyl moiety.

Embodiment B37. The oligomeric duplex of embodiment B32, wherein the duplex comprises a conjugate moiety that binds type 1 transferrin receptor (TfR1).

Embodiment B38. The oligomeric duplex of embodiment B37, wherein the conjugate moiety is selected from an antibody or fragment thereof, a protein or peptide and an aptamer capable of binding TfR1.

Embodiment B39. The oligomeric duplex of embodiment B38, wherein the conjugate moiety is a cyclic protein or cyclic peptide.

Embodiment B40. The oligomeric duplex of embodiment B37, wherein the conjugate group consists of a bicycle ligand and a conjugate linker.

Embodiment B41. The oligomeric duplex of embodiment B40, wherein the bicycle ligand comprises a peptide consisting of 13-22 linked amino acids or amino acid mimetics and a molecular scaffold, wherein each of a first, a second, and a third amino acid of the peptide comprises a reactive group, each of which separately forms a bond with the molecular scaffold, thereby forming two peptide loops attached to the molecular scaffold.

Embodiment B42. The oligomeric duplex of embodiment B41, wherein the peptide has an amino acid sequence selected from any one of SEQ ID NOs: 1071-1253 and wherein each cysteine forms a covalent bond with the molecular scaffold 1,1′,1″-(1,3,5-triazinane-1,3,5-triyl)tris(2-bromoethanone) (TATB) or the molecular scaffold 1,1′,1″-(1,3,5-triazinane-1,3,5-triyl)triprop-2-en-1-one (TATA).

Embodiment B43. The oligomeric duplex of embodiment B41, wherein the conjugate group has the structure:

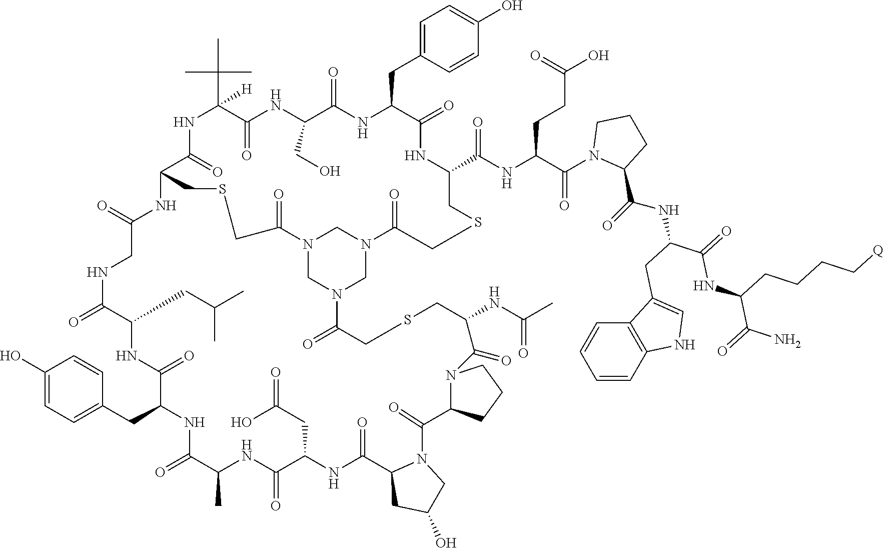

or a salt thereof wherein Q is N₃ (BCY17901, SEQ ID NO: 1045), NH₂ (BCY21757, SEQ ID NO: 1202), SH (BCY21758, SEQ ID NO: 1203), a conjugate linker, or a conjugate linker covalently connected to an oligonucleotide.

Embodiment B44. The oligomeric duplex of any one of embodiments B32-B43, wherein the conjugate group is conjugated to the second modified oligonucleotide.

Embodiment B45. The oligomeric duplex of any one of embodiments B32-B43, wherein the conjugate group is conjugated to the 5′ end or 3′ end of the second modified oligonucleotide.

Embodiment B46. The oligomeric duplex of any one of embodiments B32-B43, wherein the conjugate group is attached to the 5′-terminal nucleoside or the 3′-terminal nucleoside of the second modified oligonucleotide.

Embodiment B47. The oligomeric duplex of any one of embodiments B32-B43, wherein the conjugate group is attached to the 5′-terminal nucleoside of the second modified oligonucleotide.

Embodiment B48. The oligomeric duplex of any one of embodiments B32-B43, wherein the conjugate group is attached to the 3′-terminal nucleoside of the second modified oligonucleotide.

Embodiment B49. The oligomeric duplex of any one of embodiments B32-B48, wherein the conjugate linker of the conjugate group consists of a single bond.

Embodiment B50. The oligomeric duplex of any one of embodiments B32-B48, wherein the conjugate linker of the conjugate group is cleavable.

Embodiment B51. The oligomeric duplex of any one of embodiments B32-B50, wherein the conjugate linker comprises 1 to 3 linker-nucleosides.

Embodiment B52. The oligomeric duplex of any one of embodiments B32-B48, wherein the conjugate linker comprises a (bicyclo[6.1.0]nonyne)-formyl (BCN) moiety.

Embodiment B53. The oligomeric duplex of embodiment B52, wherein the conjugate linker comprises a 6-(BCN-carbamate)-2-(hydroxymethyl)hexyl phosphoryl moiety or a 6-(BCN-carbamate)hexyl phosphoryl moiety.

Embodiment B54. The oligomeric duplex of embodiment B41, wherein the conjugate group has the structure:

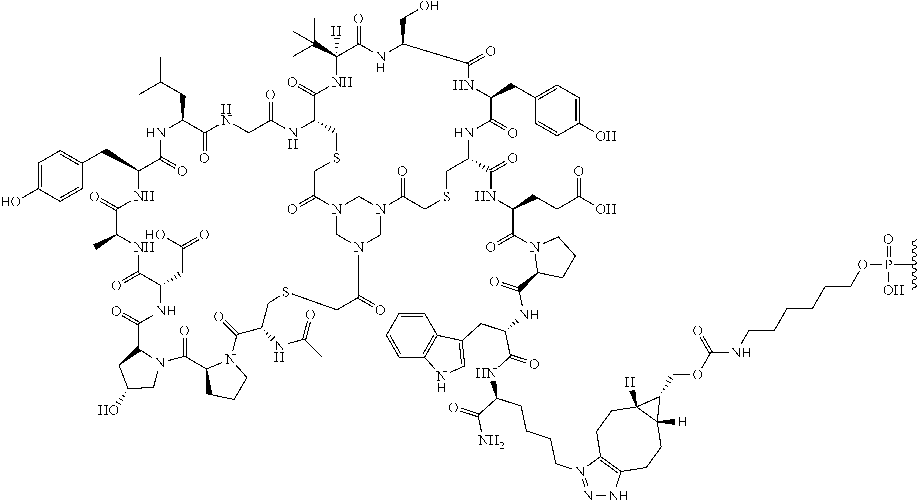

Embodiment B55. The oligomeric duplex of embodiment B41, wherein the conjugate group has the structure:

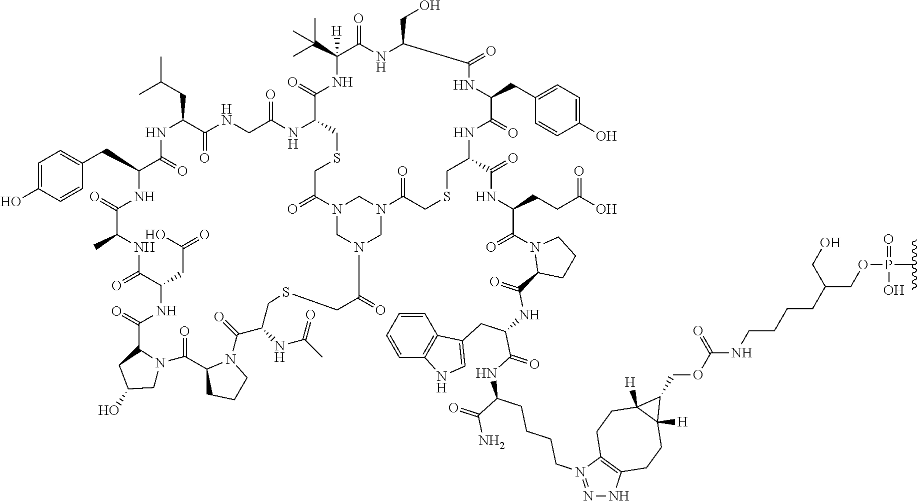

Embodiment B56. The oligomeric duplex of any one of embodiments B1-B55, wherein, the nucleobase sequence of the first modified oligonucleotide is at least 80%, at least 85%, at least 90%, at least 95%, or 100% complementary to an equal length portion within nucleobases selected from any one of 295-316, 296-317, 297-318, 298-320, 299-320, 300-321, 301-322, 302-324, 303-324, 304-325, 304-326, 305-327, 306-325, 306-328, 307-328, 308-329, 309-330, 310-331, 311-332, 312-333, 313-334, 314-335, 315-336, 316-337, 317-339, 318-339, 319-340, 320-341, 321-342, 322-344, 323-345, 324-345, 324-346, 325-345, 325-347, 326-347, 327-348, 328-349, 329-350, 330-351, 331-352, 332-353, 333-354, 334-355, 505-526, 506-528, 507-529, 508-530, 509-531, 510-531, 511-532, 512-533, 513-535, 514-535, 514-536, 515-536, 516-535, 516-537, 517-538, 518-539, 519-541, 520-541, 521-542, 522-544, 523-544, 524-546, 535-556, 536-557, 537-558, 538-559, 539-560, 540-561, 541-563, 542-563, 543-564, 544-565, 545-566, 546-567, 547-568, 548-570, 549-571, 550-572, 551-572, 552-574, 553-574, 554-575, 555-577, 556-578, 557-579, 558-580, 559-580, 560-581, 561-582, 562-583, 563-584, 595-616, 596-618, 597-618, 598-620, 599-621, 600-622, 601-623, 602-623, 603-624, 604-625, 605-627, 606-628, 607-628, 608-629, 609-630, 610-631, 611-632, 612-633, 613-635, 665-687, 666-687, 667-689, 668-689, 669-690, 670-691, 671-692, 672-694, 673-694, 674-695, 675-696, 676-697, 677-698, 678-700, 679-701, 680-702, 681-703, 682-703, 683-704, 684-705, 685-706, 686-708, 687-708, 688-709, 689-710, 690-711, 691-712, 692-714, 693-715, 1675-1696, 1676-1698, 1677-1698, 1678-1699, 1679-1700, 1680-1702, 1681-1702, 1682-1703, 1683-1705, 1684-1705, 1685-1706, 1686-1707, 1687-1709, 1688-1709, 1689-1710, 1690-1712, 1691-1713, 1692-1714, or 1693-1714 of SEQ ID NO: 1.

Embodiment B57. The oligomeric duplex of any one of embodiments B1-B55, wherein the nucleobase sequence of the first modified oligonucleotide is at least 80%, at least 85%, at least 90%, at least 95%, or 100% complementary to an equal length portion within nucleobases 304-326, 306-325, 324-346, 325-345, 514-536, 516-535 of SEQ ID NO: 1.

Embodiment B58. The oligomeric duplex of any one of embodiments B1-B55, wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases of any one of the nucleobase sequences of SEQ ID NOs: 3-314, 627-782, 939-976, 1033-1038, or 1254-1255.

Embodiment B59. The oligomeric duplex of any one of embodiments B1-B55, wherein the nucleobase sequence of the first modified oligonucleotide comprises the nucleobase sequence of any one of SEQ ID NOs: 3-314, 627-782, 939-976, 1033-1038, or 1254-1255.

Embodiment B60. The oligomeric duplex of any one of embodiments B1-B55, wherein the nucleobase sequence of the first modified oligonucleotide consists of the nucleobase sequence of any one of SEQ ID NOs: 3-314, 627-782, 939-976, 1033-1038, or 1254-1255.

Embodiment B61. The oligomeric duplex of any one of embodiments B1-B55, wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases of any one of the nucleobase sequences of SEQ ID NOs: 29,171,176, 967, 968, 971, 972, 974, 975, 1254, 1255, or 1033-1038.

Embodiment B62. The oligomeric duplex of any one of embodiments B1-B55, wherein the nucleobase sequence of the first modified oligonucleotide comprises the nucleobase sequence of any one of SEQ ID NOs: 29,171,176, 967, 968, 971, 972, 974, 975, 1254, 1255, or 1033-1038.

Embodiment B63. The oligomeric duplex of any one of embodiments B1-B55, wherein the nucleobase sequence of the first modified oligonucleotide consists of the nucleobase sequence of any one of SEQ ID NOs: 29,171,176, 967, 968, 971, 972, 974, 975, 1254, 1255, or 1033-1038.

Embodiment B64. The oligomeric duplex of any one of embodiments B1-B55, wherein the first modified oligonucleotide and the second modified oligonucleotide each independently consist of 18 to 30, 18 to 25, 18 to 24, 18 to 23, 18-22, 18-21, 18-19, 19 to 30, 19 to 25, 19 to 24, 19 to 23, 19-21, 19-20, 20 to 30, 20 to 25, 20 to 24, 20 to 23, 20 to 22, or 20 to 21 linked nucleosides.

Embodiment B65. The oligomeric duplex of any one of embodiments B1-B55, wherein the first modified oligonucleotide consists of 23 nucleosides.

Embodiment B66. The oligomeric duplex of any one of embodiments B1-B65, wherein the first modified oligonucleotide has a sugar motif (5′ to 3′) selected from among: efyyydyyyyyyyfyfyyyyyee, efyyydyyyyyyyfyfyyyyyyy, e[FHNA]yyydyyyyyyyfyfyyyyyee, e[FHNA]yyydyyyyyyyfyfyyyyyyy, efyydydyyyyyyfyfyyyyyee, efyydydyyyyyyfyfyyyyyyy, e[FHNA]yydydyyyyyyfyfyyyyyee, e[FHNA]yydydyyyyyyfyfyyyyyyy, efyyyfyyyyyyyfyfyyyyyyy, efyyyfyyyyyyyfyfyyyyyey, efyyyyyyyyyyyfyfyyyyyee, efyyyfyyyyyyyfyfyyyyyee, efyyyyyyyyyyyfyyyyyyyee, efyyyyyyyyyyyfyyyyyyyyy, e[FHNA]yyyfyyyyyyyfyfyyyyyee, e[FHNA]yyyfyyyyyyyfyfyyyyyyy, efyyydyyyyyyydydyyyyyee, efyyydyyyyyyydydyyyyyyy, efyydydyyyyyyfyfyyyyyyy, efyyyfyyyyyyyfyyyyyyyee, efyyyfyyyyyyyfyyyyyyyyy, efyyyyyyyyyyyfyfyyyyyee, efyyyyyyyyyyyfyfyyyyyyy, e[FHNA]yydydyyyyfyfyfyyyyyyy, efyyyfyyyyyyyfydyyyyyyy, efyyyfyyyyyyyfydyyyyyee, efyyyfyyyyyyyfyfyyyee, and efyyyfyyyyyyyfyfyyyyy, wherein each “d” represents a 2′-β-D-deoxyribosyl sugar moiety, each “y” represents a 2′-OMe sugar moiety, each “e” represents a 2′-MOE sugar moiety, each “f” represents a 2′-fluoro sugar moiety, and each “[FHNA]” represents a 3′-fluoro-hexitol sugar moiety.

Embodiment B67. The oligomeric duplex of any one of embodiments B1-B65, wherein the first modified oligonucleotide has a sugar motif (5′ to 3′) selected from among: efyydydyyyyyyfyfyyyyyee, efyyydyyyyyyyfyfyyyyyee, efyydydyyyyyyfyfyyyyyyy, e[FHNA]yydydyyyyfyfyfyyyyyyy, efyyyyyyyyyyyfyfyyyyyee and efyyydyyyyyyyfyfyyyyyyy.

Embodiment B68. The oligomeric duplex of any one of embodiments B1-B67, wherein the first modified oligonucleotide comprises at least one modified internucleoside linkage.

Embodiment B69. The oligomeric duplex of embodiment B68, wherein at least one modified internucleoside linkage is a phosphorothioate or mesyl phosphoramidate internucleoside linkage.

Embodiment B70. The oligomeric duplex of embodiment B68, wherein fewer than 50%, fewer than 40%, fewer than 30%, fewer than 20%, or fewer than 10% of the internucleoside linkages of the first modified oligonucleotide are modified internucleoside linkages.

Embodiment B71. The oligomeric duplex of any one of embodiments B1-70, wherein each internucleoside linkage of the first modified oligonucleotide is independently selected from a phosphodiester internucleoside linkage and a phosphorothioate internucleoside linkage.

Embodiment B72. The oligomeric duplex of embodiment B71, wherein the internucleoside linkages between the first and second nucleosides and the second and third nucleosides counting from the 5′ end of the first modified oligonucleotide are modified internucleoside linkages.

Embodiment B73. The oligomeric duplex of embodiment B71, wherein the internucleoside linkages between the first and second nucleosides and the second and third nucleosides counting from the 3′ end of the first modified oligonucleotide are modified internucleoside linkages.

Embodiment B74. The oligomeric duplex of embodiment B72, wherein the internucleoside linkages between the first and second nucleosides and the second and third nucleosides counting from the 5′ end of the first modified oligonucleotide, and the internucleoside linkages between the first and second nucleosides and the second and third nucleosides counting from the 3′ end of the first modified oligonucleotide are modified internucleoside linkages.

Embodiment B75. The oligomeric duplex of any one of embodiments B71-B74, wherein the modified internucleoside linkages are phosphorothioate internucleoside linkages.

Embodiment B76. The oligomeric duplex of embodiment B74 or embodiment B75, wherein all other internucleoside linkages in the first modified oligonucleotide are phosphodiester internucleoside linkages.

Embodiment B77. The oligomeric duplex of embodiment B76, wherein the first modified oligonucleotide has an internucleoside linkage motif of ssooooooooooooooooooss and a sugar motif (5′ to 3′) selected from among: efyydydyyyyyyfyfyyyyyee, efyyydyyyyyyyfyfyyyyyee, efyydydyyyyyyfyfyyyyyyy, e[FHNA]yydydyyyyfyfyfyyyyyyy, efyyyyyyyyyyyfyfyyyyyee and efyyydyyyyyyyfyfyyyyyyy; wherein each “d” represents a 2′-β-D-deoxyribosyl sugar moiety, each “y” represents a 2′-OMe sugar moiety, each “e” represents a 2′-MOE sugar moiety, each “f” represents a 2′-fluoro sugar moiety, each “[FHNA]” represents a 3′-fluoro-hexitol sugar moiety, each “o” represents a phosphodiester internucleoside linkage and each “s” represents a phosphorothioate internucleoside linkage.

Embodiment B78. The oligomeric duplex of any one of embodiments B1-B77, wherein fewer than 30%, fewer than 25%, fewer than 20%, fewer than 15%, fewer than 14%, fewer than 13%, fewer than 12%, fewer than 10%, or fewer than 5% of the nucleosides in the second modified oligonucleotide comprise a fluorine.

Embodiment B79. The oligomeric duplex of any one of embodiments B1-B77, wherein no more than 4 nucleosides, no more than 3 nucleosides, no more than 2 nucleosides, or no more than 1 nucleoside in the second modified oligonucleotide comprise a modified sugar moiety comprising a fluorine.

Embodiment B80. The oligomeric duplex of any one of embodiments B1-B77, wherein none of the nucleosides before the seventh or after the eleventh nucleoside counting from the 5′ end of the second modified oligonucleotide comprise a modified sugar moiety comprising a fluorine.

Embodiment B81. The oligomeric duplex of any one of embodiments B1-B77, wherein one or more of the seventh, ninth and eleventh nucleosides counting from the 5′ end of the second modified oligonucleotide comprises a modified sugar moiety comprising a fluorine.

Embodiment B82. The oligomeric duplex embodiment B81, wherein two or more of the seventh, ninth and eleventh nucleosides counting from the 5′ end of the second modified oligonucleotide comprise a modified sugar moiety comprising a fluorine.

Embodiment B83. The oligomeric duplex embodiment B81, wherein the seventh, ninth and eleventh nucleosides counting from the 5′ end of the second modified oligonucleotide comprise a modified sugar moiety comprising a fluorine.

Embodiment B84. The oligomeric duplex of any one of embodiments B1-B80, wherein one or both of the tenth and eleventh nucleosides counting from the 5′ end of the second modified oligonucleotide comprises a modified sugar moiety comprising a fluorine.

Embodiment B85. The oligomeric duplex of any one of embodiments B77-B84, wherein the modified sugar moiety comprising a fluorine is a 2′-fluoro sugar moiety.

Embodiment B86. The oligomeric duplex of any one of embodiments B1-B85, wherein fewer than 30%, fewer than 25%, fewer than 20%, fewer than 15%, fewer than 14%, fewer than 13%, fewer than 12%, fewer than 10%, fewer than 5%, or fewer than 1% of the total nucleosides in the oligomeric duplex comprise a fluorine.

Embodiment B87. The oligomeric duplex of any one of embodiments B1-B86, wherein one or more of the nucleosides of the second modified oligonucleotide independently comprises a sugar surrogate or a bicyclic sugar moiety.

Embodiment B88. The oligomeric duplex of embodiment B87, wherein the bicyclic sugar moiety comprises a 2′-4′ bridge selected from —O—CH₂—; and —O—CH(CH₃)—.

Embodiment B89. The oligomeric duplex of any one of embodiments B1-B88, wherein one or more of the nucleosides of the second modified oligonucleotide comprises a 2′-OMe sugar moiety.

Embodiment B90. The oligomeric duplex of embodiment B89, wherein at least 10%, at least 20%, at least 30%, at least 40%, at least 50%, or at least 60%, or at least 65%, or at least 70%, or at least 80%, or at least 85%, or at least 90% of the nucleosides of the second modified oligonucleotide comprise a 2′-OMe sugar moiety.

Embodiment B91. The oligomeric duplex of embodiment B89 or embodiment B90, wherein the one or more nucleosides comprising a 2′-OMe sugar moiety are in a region of the sequence of the second modified oligonucleotide between and including the third and nineteenth nucleosides counting from the 5′ end of the second modified oligonucleotide.

Embodiment B92. The oligomeric duplex of embodiment B89 or embodiment B90, wherein the 3′-terminal nucleoside of the second modified oligonucleotide comprises a 2′-OMe sugar moiety and/or the nucleoside immediately 5′ of the 3′-terminal nucleoside of the second modified oligonucleotide comprises a 2′-OMe sugar moiety.

Embodiment B93. The oligomeric duplex of any one of embodiments B89, B90 and B92, wherein the 5′-terminal nucleoside of the second modified oligonucleotide comprises a 2′-OMe sugar moiety and/or the nucleoside immediately 3′ of the 5′-terminal nucleoside of the second modified oligonucleotide comprises a 2′-OMe sugar moiety.

Embodiment B94. The oligomeric duplex of embodiment B89 or embodiment B90, wherein the 5′-terminal nucleoside, the nucleoside immediately 3′ of the 5′-terminal nucleoside, the 3′-terminal nucleoside, and the nucleoside immediately 5′ of the 3′-terminal nucleoside of the second modified oligonucleotide comprises a 2′-OMe sugar moiety.

Embodiment B95. The oligomeric duplex of any one of embodiments B1-B94, wherein one or more of the nucleosides of the second modified oligonucleotide comprise a 2′-MOE sugar moiety.

Embodiment B96. The oligomeric duplex of any one of embodiments B1-B93, wherein the 3′-terminal nucleoside of the second modified oligonucleotide comprises a 2′-MOE sugar moiety and/or the nucleoside immediately 5′ of the 3′-terminal nucleoside of the second modified oligonucleotide comprises a 2′-MOE sugar moiety.

Embodiment B97. The oligomeric duplex of any one of embodiments B1-B93, B95 and B96, wherein the 5′-terminal nucleoside of the second modified oligonucleotide comprises a 2′-MOE sugar moiety and/or the nucleoside immediately 3′ of the 5′-terminal nucleoside of the second modified oligonucleotide comprises a 2′-MOE sugar moiety.

Embodiment B98. The oligomeric duplex of any one of embodiments B1-B91, wherein the 5′-terminal nucleoside, the nucleoside immediately 3′ of the 5′-terminal nucleoside, the 3′-terminal nucleoside, and the nucleoside immediately 5′ of the 3′-terminal nucleoside of the second modified oligonucleotide comprises a 2′-MOE sugar moiety.

Embodiment B99. The oligomeric duplex of any one of embodiments B1-B98, wherein the second modified oligonucleotide has a sugar motif (5′ to 3′) selected from among: eeyyyyfyfyfyyyyyyyyee, eeyyyyfyfyfyyyyyyyyyy, yyyyyfyfyfyyyyyyyyee, yyyyyfyfyfyyyyyyyyyy, eeyyyyfyfffyyyyyyyyee, eeyyyyfyfffyyyyyyyyyy, yyyyyyfyfffyyyyyyyyee, yyyyyyfyfffyyyyyyyyyy, eeyyyyyyyffyyyyyyyyee, eeyyyyyyyffyyyyyyyyyy, yyyyyyyyyffyyyyyyyyee, yyyyyyyyyffyyyyyyyyyy, eeyyyyyffyyyyyyyyee, eeyyyyyffyyyyyyyyyy, yyyyyyyffyyyyyyyyee, yyyyyyyffyyyyyyyyyy, yyyyyyfyfffyyyyyyyy, eeyyyyfyfffyyyyyyee, eeyyyyfyfffyyyyyyyy, yyyyyyfyfffyyyyyyee, eeyyfyfffyyyyyyyyee, eeyyfyfffyyyyyyyyyy, yyyyfyfffyyyyyyyyee, yyyyfyfffyyyyyyyyyy, eeyyyyyyyydyyyyyyyyee, eeyyyyyyyydyyyyyyyyyy, yyyyyyyyyydyyyyyyyyee, yyyyyyyyyydyyyyyyyyyy, eeyyyyyyyyyyyyyyyyyee, eeyyyyyyyyyyyyyyyyyyy, yyyyyyyyyyyyyyyyyyyee, and yyyyyyyyyyyyyyyyyyyyy wherein each “y” represents a 2′-OMe sugar moiety, each “e” represents a 2′-MOE sugar moiety, each “f” represents a 2′-fluoro sugar moiety, and each “d” represents a 2′-β-D-deoxyribosyl sugar moiety.

Embodiment B100. The oligomeric duplex of any one of embodiments B1-B98, wherein the second modified oligonucleotide has a sugar motif (5′ to 3′) selected from among: eeyyyyyyyffyyyyyyyyee, eeyyyyfyfyfyyyyyyyyee, yyyyyyyyyffyyyyyyyyyy, and yyyyyyfyfyfyyyyyyyyyy, wherein each “y” represents a 2′-OMe sugar moiety, each “e” represents a 2′-MOE sugar moiety, and each “f” represents a 2′-fluoro sugar moiety.

Embodiment B101. The oligomeric duplex of any one of embodiments B1-B100, wherein the nucleobase of the 3′-terminal nucleoside and/or of the nucleoside immediately 5′ of the 3′-terminal nucleoside of the second modified oligonucleotide is an adenine.

Embodiment B102. The oligomeric duplex of any one of embodiments B1-B100, wherein the nucleobase of the 3′-terminal nucleoside is an adenine and the nucleobase of the nucleoside immediately 5′ of the 3′-terminal nucleoside of the second modified oligonucleotide is a thymine or uracil.

Embodiment B103. The oligomeric duplex of any one of embodiments B1-B100, wherein the second modified oligonucleotide comprises at least one modified internucleoside linkage.

Embodiment B104. The oligomeric duplex of embodiment B103, wherein at least one modified internucleoside linkage is a phosphorothioate or mesyl phosphoramidate internucleoside linkage.

Embodiment B105. The oligomeric duplex of embodiment B103, wherein fewer than 50%, fewer than 40%, fewer than 30%, fewer than 20%, or fewer than 10% of the internucleoside linkages of the second modified oligonucleotide are modified internucleoside linkages.

Embodiment B106. The oligomeric duplex of any one of embodiments B103-B105, wherein each internucleoside linkage of the second modified oligonucleotide is independently selected from a phosphodiester internucleoside linkage and a phosphorothioate internucleoside linkage.

Embodiment B107. The oligomeric duplex of any one of embodiments B103-B105, wherein the internucleoside linkages between the first and second nucleosides and the second and third nucleosides counting from the 5′ end of the second modified oligonucleotide are modified internucleoside linkages.

Embodiment B108. The oligomeric duplex of any one of embodiments B103-B105 and B107, wherein the internucleoside linkages between the first and second nucleosides and the second and third nucleosides counting from the 3′ end of the second modified oligonucleotide are modified internucleoside linkages.

Embodiment B109. The oligomeric duplex of any one of embodiments B103-B105, wherein the internucleoside linkages between the first and second nucleosides and the second and third nucleosides counting from the 5′ end of the second modified oligonucleotide, and the internucleoside linkages between the first and second nucleosides and the second and third nucleosides counting from the 3′ end of the second modified oligonucleotide are modified internucleoside linkages.

Embodiment B110. The oligomeric duplex of any one of embodiments B103-B109, wherein the modified internucleoside linkages are phosphorothioate internucleoside linkages.

Embodiment B111. The oligomeric duplex of embodiment B110, wherein all other internucleoside linkages in the second modified oligonucleotide are phosphodiester internucleoside linkages.

Embodiment B112. The oligomeric duplex of embodiment B111, wherein the second modified oligonucleotide has an internucleoside linkage motif of ssooooooooooooooooss and a sugar motif (5′ to 3′) selected from among: eeyyyyyyyffyyyyyyyyee, eeyyyyfyfyfyyyyyyyyee, yyyyyyyyyffyyyyyyyyyy, and yyyyyyfyfyfyyyyyyyyyy; wherein each ““y” represents a 2′-OMe sugar moiety, each “e” represents a 2′-MOE sugar moiety, each “f” represents a 2′-fluoro sugar moiety, each “o” represents a phosphodiester internucleoside linkage and each “s” represents a phosphorothioate internucleoside linkage.

Embodiment B113. The oligomeric duplex of any one of embodiments B1-B112, wherein the nucleobase sequence of the second modified oligonucleotide comprises least 8, at least 9, at least 10, at least 11, at least 12, at least 13, at least 14, at least 15, at least 16, at least 17, at least 18, at least 19, at least 20, or at least 21 contiguous nucleobases of any one of the nucleobase sequences of SEQ ID NOs: 315-626, 783-938, 977-1016, or 1027-1032.

Embodiment B114. The oligomeric duplex of any one of embodiments B1-B112, wherein the nucleobase sequence of the second modified oligonucleotide comprises the nucleobase sequence of any one of SEQ ID NOs: 315-626, 783-938, 977-1016, or 1027-1032.

Embodiment B115. The oligomeric duplex of any one of embodiments B1-B112, wherein the nucleobase sequence of the second modified oligonucleotide consists of the nucleobase sequence of any one of SEQ ID NOs: 315-626, 783-938, 977-1016, or 1027-1032.

Embodiment B116. The oligomeric duplex of any one of embodiments B1-B112, wherein the nucleobase sequence of the second modified oligonucleotide comprises at least 8, at least 9, at least 10, at least 11, at least 12, at least 13, at least 14, at least 15, or at least 16 contiguous nucleobases of any one of the nucleobase sequences of SEQ ID NOs: 341, 483, 488, 1011-1016, or 1027-1032.

Embodiment B117. The oligomeric duplex of any one of embodiments B1-B112, wherein the nucleobase sequence of the second modified oligonucleotide comprises the nucleobase sequence of any one of SEQ ID NOs: 341, 483, 488, 1011-1016, or 1027-1032.

Embodiment B118. The oligomeric duplex of any one of embodiments B1-B112, wherein the nucleobase sequence of the second modified oligonucleotide consists of the nucleobase sequence of any one of SEQ ID NOs: 341, 483, 488, 1011-1016, or 1027-1032.

Embodiment B119. An oligomeric duplex comprising:
(1) a first oligomeric compound comprising a first modified oligonucleotide consisting of 18 to 30 contiguous linked nucleosides that is at least 80%, at least 85%, at least 90%, at least 95% or at least 95% complementary or 100% complementary to an equal length portion within nucleobases selected from any one of 295-316, 296-317, 297-318, 298-320, 299-320, 300-321, 301-322, 302-324, 303-324, 304-325, 304-326, 305-327, 306-325, 306-328, 307-328, 308-329, 309-330, 310-331, 311-332, 312-333, 313-334, 314-335, 315-336, 316-337, 317-339, 318-339, 319-340, 320-341, 321-342, 322-344, 323-345, 324-345, 324-346, 325-345, 325-347, 326-347, 327-348, 328-345, 328-349, 329-350, 330-351, 331-352, 332-353, 333-354, 334-355, 505-526, 506-528, 507-529, 508-530, 509-531, 510-531, 511-532, 512-533, 513-535, 514-535, 514-536, 515-536, 516-535, 516-537, 517-538, 518-539, 519-541, 520-541, 521-542, 522-544, 523-544, 524-546, 535-556, 536-557, 537-558, 538-559, 539-560, 540-561, 541-563, 542-563, 543-564, 544-565, 545-566, 546-567, 547-568, 548-570, 549-571, 550-572, 551-572, 552-574, 553-574, 554-575, 555-577, 556-578, 557-579, 558-580, 559-580, 560-581, 561-582, 562-583, 563-584, 595-616, 596-618, 597-618, 598-620, 599-621, 600-622, 601-623, 602-623, 603-624, 604-625, 605-627, 606-628, 607-628, 608-629, 609-630, 610-631, 611-632, 612-633, 613-635, 665-687, 666-687, 667-689, 668-689, 669-690, 670-691, 671-692, 672-694, 673-694, 674-695, 675-696, 676-697, 677-698, 678-700, 679-701, 680-702, 681-703, 682-703, 683-704, 684-705, 685-706, 686-708, 687-708, 688-709, 689-710, 690-711, 691-712, 692-714, 693-715, 1675-1696, 1676-1698, 1677-1698, 1678-1699, 1679-1700, 1680-1702, 1681-1702, 1682-1703, 1683-1705, 1684-1705, 1685-1706, 1686-1707, 1687-1709, 1688-1709, 1689-1710, 1690-1712, 1691-1713, 1692-1714, or 1693-1714 of SEQ ID NO: 1, and
(2) a second oligomeric compound comprising a second modified oligonucleotide consisting of 16 to 28 contiguous linked nucleosides that is at least 90% complementary to an equal length portion of the first modified oligonucleotide;
   wherein:
(a) each of the nucleosides of the first modified oligonucleotide and each of the nucleosides of the second modified oligonucleotide comprise a modified sugar moiety or a sugar surrogate, (b) at least one of the modified sugar moiety or sugar surrogate of the first modified oligonucleotide comprises a fluorine, and
(c) fewer than 40% of the nucleosides of the first modified oligonucleotide comprise a fluorine.

Embodiment B120. An oligomeric duplex comprising, (1) a first oligomeric compound comprising a first modified oligonucleotide consisting of 18 to 30 contiguous linked nucleosides, wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases of any one of the nucleobase sequences of SEQ ID NOs: 3-314, 627-782, 939-976, 1033-1038 or 1254-1255; and (2) a second oligomeric compound comprising a second modified oligonucleotide consisting of 16 to 28 contiguous linked nucleosides that is at least 90% complementary to an equal length portion of the first modified oligonucleotide; wherein: (a) each of the nucleosides of the first modified oligonucleotide and each of the nucleosides of the second modified oligonucleotide comprise a modified sugar moiety or a sugar surrogate, (b) at least one of the modified sugar moiety or sugar surrogate of the first modified oligonucleotide comprises a fluorine, and (c) fewer than 40% of the nucleosides of the first modified oligonucleotide comprise a fluorine.

Embodiment B121. The oligomeric duplex of embodiment B120, wherein the nucleobase sequence of the first modified oligonucleotide comprises the nucleobase sequence of any one of SEQ ID NOs: 3-314, 627-782, 939-976, 1033-1038, or 1254-1255.

Embodiment B122. The oligomeric duplex of embodiment B120, wherein the nucleobase sequence of the first modified oligonucleotide consists of the nucleobase sequence of any one of SEQ ID NOs: 3-314, 627-782, 939-976, 1033-1038, or 1254-1255.

Embodiment B123. The oligomeric duplex of embodiment B120, wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases of any one of the nucleobase sequences of SEQ ID NOs: 29, 171, 176, 967, 968, 971, 972, 974, 975, 1254, 1255, or 1033-1038.

Embodiment B124. The oligomeric duplex of embodiment B120, wherein the nucleobase sequence of the first modified oligonucleotide comprises the nucleobase sequence of any one of SEQ ID NOs: 29,171,176, 967, 968, 971, 972, 974, 975, 1254, 1255, or 1033-1038.

Embodiment B125. The oligomeric duplex of embodiment B120, wherein the nucleobase sequence of the first modified oligonucleotide consists of the nucleobase sequence of any one of SEQ ID NOs: 29,171,176, 967, 968, 971, 972, 974, 975, 1254, 1255, or 1033-1038.

Embodiment B126. The oligomeric duplex of embodiment B120, wherein the nucleobase sequence of the second modified oligonucleotide comprises least 8, at least 9, at least 10, at least 11, at least 12, at least 13, at least 14, at least 15, at least 16, at least 17, at least 18, at least 19, at least 20, or at least 21 contiguous nucleobases of any one of the nucleobase sequences of SEQ ID NOs: 315-626, 783-938, 977-1016, or 1027-1032.

Embodiment B127. The oligomeric duplex of embodiment B121, wherein the nucleobase sequence of the second modified oligonucleotide comprises the nucleobase sequence of any one of SEQ ID NOs: 315-626, 783-938, 977-1016, or 1027-1032.

Embodiment B128. The oligomeric duplex of embodiment B122, wherein the nucleobase sequence of the second modified oligonucleotide consists of the nucleobase sequence of any one of SEQ ID NOs: 315-626, 783-938, 977-1016, or 1027-1032.

Embodiment B129. The oligomeric duplex of embodiment B123, wherein the nucleobase sequence of the second modified oligonucleotide comprises at least 8, at least 9, at least 10, at least 11, at least 12, at least 13, at least 14, at least 15, at least 16, at least 17, at least 18, at least 19, at least 20, or at least 21 contiguous nucleobases of any one of the nucleobase sequences of SEQ ID NOs: 341, 483, 488, 1011-1016, or 1027-1032.

Embodiment B130. The oligomeric duplex of embodiment B124, wherein the nucleobase sequence of the second modified oligonucleotide comprises the nucleobase sequence of any one of SEQ ID NOs: 341, 483, 488, 1011-1016, or 1027-1032.

Embodiment B131. The oligomeric duplex of embodiment B125, wherein the nucleobase sequence of the second modified oligonucleotide consists of the nucleobase sequence of any one of SEQ ID NOs: 341, 483, 488, 1011-1016, or 1027-1032.

Embodiment B132. The oligomeric duplex of any one of embodiments B119-B131, wherein none of the modified sugar moieties or sugar surrogates of the nucleosides of the second modified oligonucleotide comprises a fluorine.

Embodiment B133. The oligomeric duplex of any one of embodiments B119-B131, wherein at least one of the modified sugar moieties or sugar surrogates of the second modified oligonucleotide comprises a fluorine and wherein fewer than 40% of the nucleosides of the second modified oligonucleotide comprises a fluorine.

Embodiment B134. The oligomeric duplex of any one of embodiments B119-B131, wherein two, three or four of the nucleosides of the second modified oligonucleotide independently comprise a modified sugar moiety comprising a fluorine or a sugar surrogate comprising a fluorine.

Embodiment B135. The oligomeric duplex of any one of embodiments B119-B134, wherein two, three or four of the nucleosides of the first modified oligonucleotide independently comprise a modified sugar moiety comprising a fluorine or a sugar surrogate comprising a fluorine.

Embodiment B136. The oligomeric duplex of any one of embodiments B119-B135 wherein none of the nucleosides of the second modified oligonucleotide is a 2′-deoxynucleoside and/or none of the nucleosides of the first modified oligonucleotide is a 2′-deoxynucleoside.

Embodiment B137. The oligomeric duplex of any one of embodiments B119-B136 wherein all of the nucleosides of the second modified oligonucleotide comprise a modified ribosyl sugar moiety.

Embodiment B138. The oligomeric duplex of any one of embodiments B119-B137, wherein one, two or three of the nucleosides of the first modified oligonucleotide is/are a 2′-deoxynucleoside.

Embodiment B139. The oligomeric duplex of any one of embodiments B119-B138, wherein:
(a) the 5′- and 3′-terminal nucleosides, the nucleoside immediately adjacent to the 5′-terminal nucleoside, and the nucleoside immediately adjacent to the 3′-terminal nucleoside of the second modified oligonucleotide comprise a 2′-MOE sugar moiety,
(b) the 5′- and 3′-terminal nucleosides and the nucleoside immediately 5′ of the 3′-terminal nucleoside of the first modified oligonucleotide comprise a 2′-MOE sugar moiety, and/or
(c) any nucleoside in the first and second modified oligonucleotides that does not comprise a fluorine or a 2′-MOE sugar moiety comprises a 2′-OMe sugar moiety.

Embodiment B140. The oligomeric duplex of any one of embodiments B119-B139, wherein no more than four of the nucleosides of the first modified oligonucleotide comprise a modified sugar moiety comprising a fluorine or a sugar surrogate comprising a fluorine.

Embodiment B141. The oligomeric duplex of embodiment B140, wherein the first modified oligonucleotide has a sugar motif (5′ to 3′) selected from among: efyyyfyyyyyyyfyfyyyyyee, efyyyfyyyyyyyfyfyyyyyyy, e[FHNA]yyyfyyyyyyyfyfyyyyyee, e[FHNA]yyyfyyyyyyyfyfyyyyyyy, efyyyfyyyyyyyfyfyyyyyey, e[FHNA]yydydyyyyfyfyfyyyyyyy and efyyyfyyyyyyyfyfyyyee; wherein each “d” represents a 2′-β-D-deoxyribosyl sugar moiety, each “y” represents a 2′-OMe sugar moiety, each “e” represents a 2′-MOE sugar moiety, each “f” represents a 2′-fluoro sugar moiety, and each “[FHNA]” represents a 3′-fluoro-hexitol sugar moiety.

Embodiment B142. The oligomeric duplex of any one of embodiments B119-B139, wherein no more than three of the nucleosides of the first modified oligonucleotide comprise a modified sugar moiety comprising a fluorine or a sugar surrogate comprising a fluorine.

Embodiment B143. The oligomeric duplex of embodiment B142 wherein the first modified oligonucleotide has a sugar motif (5′ to 3′) selected from among: efyyydyyyyyyyfyfyyyyyee, efyyydyyyyyyyfyfyyyyyyy, e[FHNA]yyydyyyyyyyfyfyyyyyee, e[FHNA]yyydyyyyyyyfyfyyyyyyy, efyydydyyyyyyfyfyyyyyee, efyydydyyyyyyfyfyyyyyyy, e[FHNA]yydydyyyyyyfyfyyyyyee, e[FHNA]yydydyyyyyyfyfyyyyyyy, efyyyyyyyyyyyyfyyyyyee, efyyyfyyyyyyyfyyyyyyyyy, efyyyfyyyyyyyfydyyyyyee, efyyyfyyyyyyyfydyyyyyyy, efyyyfyyyyyyyfydyyyyyee, efyyyfyyyyyyyfydyyyyyyy; wherein each “d” represents a 2′-β-D-deoxyribosyl sugar moiety, each “y” represents a 2′-OMe sugar moiety, each “e” represents a 2′-MOE sugar moiety, each “f” represents a 2′-fluoro sugar moiety, and each “[FHNA]” represents a 3′-fluoro-hexitol sugar moiety.

Embodiment B144. The oligomeric duplex of any one of embodiments B119-B139, wherein no more than two of the nucleosides of the first modified oligonucleotide comprise a modified sugar moiety comprising a fluorine or a sugar surrogate comprising a fluorine.

Embodiment B145. The oligomeric duplex of embodiment B144 wherein the first modified oligonucleotide has a sugar motif (5′ to 3′) selected from among: efyyyyyyyyyyyfyyyyyyyee, efyyyyyyyyyyyfyyyyyyyyy, e[FHNA]yyyyyyyyyyy[FHNA]yyyyyyyee, and e[FHNA]yyyyyyyyyyy[FHNA]yyyyyyyyy; wherein each “y” represents a 2′-OMe sugar moiety, each “e” represents a 2′-MOE sugar moiety, each “f” represents a 2′-fluoro sugar moiety, and each “[FHNA]” represents a 3′-fluoro-hexitol sugar moiety.

Embodiment B146. The oligomeric duplex of any one of embodiments B119-B139, wherein only one of the nucleosides of the first modified oligonucleotide comprises a modified sugar moiety comprising a fluorine or a sugar surrogate comprising a fluorine.

Embodiment B147. The oligomeric duplex of embodiment B146 wherein the first modified oligonucleotide has a sugar motif (5′ to 3′) selected from among: efyyydyyyyyyydydyyyyyyy, efyyydyyyyyyydydyyyyyee, e[FHNA]yyydyyyyyyydydyyyyyyy, and e[FHNA]yyydyyyyyyydydyyyyyee; wherein each “d” represents a 2′-β-D-deoxyribosyl sugar moiety, each “y” represents a 2′-OMe sugar moiety, each “e” represents a 2′-MOE sugar moiety, each “f” represents a 2′-fluoro sugar moiety, and each “[FHNA]” represents a 3′-fluoro-hexitol sugar moiety.

Embodiment B148. The oligomeric duplex of any one of embodiments B119-B144 and B146, wherein no more than two of the nucleosides of the first modified oligonucleotide are 2′-deoxynucleosides.

Embodiment B149. The oligomeric duplex of embodiment B148 wherein the first modified oligonucleotide has a sugar motif (5′ to 3′) selected from among: efyydydyyyyyyfyfyyyyyee, efyydydyyyyyyfyfyyyyyyy, e[FHNA]yydydyyyyyyfyfyyyyyee, and e[FHNA]yydydyyyyyyfyfyyyyyyy; wherein each “d” represents a 2′-β-D-deoxyribosyl sugar moiety, each “y” represents a 2′-OMe sugar moiety, each “e” represents a 2′-MOE sugar moiety, each “f” represents a 2′-fluoro sugar moiety, and each “[FHNA]” represents a 3′-fluoro-hexitol sugar moiety.

Embodiment B150. The oligomeric duplex of any one of embodiments B119-B140, B143 and B146, wherein only one of the nucleosides of the first modified oligonucleotide is a 2′-deoxynucleoside.

Embodiment B151. The oligomeric duplex of embodiment B150, wherein the first modified oligonucleotide has a sugar motif (5′ to 3′) selected from among: efyyydyyyyyyyfyfyyyyyee, efyyydyyyyyyyfyfyyyyyyy, e[FHNA]yyydyyyyyyyfyfyyyyyee, e[FHNA]yyydyyyyyyyfyfyyyyyyy, eyyyfyyyyyyyfydyyyyyyy, efyyyfyyyyyyyfydyyyyyee, eyyy[FHNA]yyyyyyyfydyyyyyyy, and e[FHNA]yyyfyyyyyyyfydyyyyyee; wherein each “d” represents a 2′-β-D-deoxyribosyl sugar moiety, each “y” represents a 2′-OMe sugar moiety, each “e” represents a 2′-MOE sugar moiety, each “f” represents a 2′-fluoro sugar moiety, and each “[FHNA]” represents a 3′-fluoro-hexitol sugar moiety.

Embodiment B152. The oligomeric duplex of any one of embodiments B119-B151, wherein no more than four of the nucleosides of the second modified oligonucleotide comprise a modified sugar moiety comprising a fluorine or a sugar surrogate comprising a fluorine.

Embodiment B153. The oligomeric duplex of embodiment B152, wherein the second modified oligonucleotide has a sugar motif (5′ to 3′) selected from among: yyyyyyfyfffyyyyyyyyyy, eeyyyyfyfffyyyyyyyyee, yyyyyyfyfffyyyyyyyy, eeyyyyfyfffyyyyyyee, yyyyfyfffyyyyyyyyyy, and eeyyfyfffyyyyyyyyee; wherein each “y” represents a 2′-OMe sugar moiety, each “e” represents a 2′-MOE sugar moiety, and each “f” represents a 2′-fluoro sugar moiety.

Embodiment B154. The oligomeric duplex of any one of embodiments B119-B151, wherein no more than three of the nucleosides of the second modified oligonucleotide comprise a modified sugar moiety comprising a fluorine or a sugar surrogate comprising a fluorine.

Embodiment B155. The oligomeric duplex of embodiment B154, wherein the second modified oligonucleotide has a sugar motif (5′ to 3′) selected from among: eeyyyyfyfyfyyyyyyyyee and yyyyyfyfyfyyyyyyyyyy; wherein each “y” represents a 2′-OMe sugar moiety, each “e” represents a 2′-MOE sugar moiety, and each “f” represents a 2′-fluoro sugar moiety.

Embodiment B156. The oligomeric duplex of any one of embodiments B119-B151, wherein no more than two of the nucleosides of the second modified oligonucleotide comprise a modified sugar moiety comprising a fluorine or a sugar surrogate comprising a fluorine.

Embodiment B157. The oligomeric duplex of embodiment B156, wherein the second modified oligonucleotide has a sugar motif (5′ to 3′) selected from among: eeyyyyyyyffyyyyyyyyee, yyyyyyyyyffyyyyyyyyyy, eeyyyyyffyyyyyyyyee, and yyyyyyyffyyyyyyyyyy; wherein each “y” represents a 2′-OMe sugar moiety, each “e” represents a 2′-MOE sugar moiety, and each “f” represents a 2′-fluoro sugar moiety.

Embodiment B158. The oligomeric duplex of any one of embodiments B119-B151, wherein none of the nucleosides of the second modified oligonucleotide comprises a modified sugar moiety comprising a fluorine or a sugar surrogate comprising a fluorine.

Embodiment B159. The oligomeric duplex of embodiment B158, wherein the second modified oligonucleotide has a sugar motif (5′ to 3′) selected from among: yyyyyyyyyyyyyyyyyyyyy, eeyyyyyyyyyyyyyyyyyee, yyyyyyyyyydyyyyyyyyyy, and eeyyyyyyyydyyyyyyyyee; wherein each “y” represents a 2′-OMe sugar moiety, each “e” represents a 2′-MOE sugar moiety, and each “d” represents a 2′-β-D-deoxyribosyl sugar moiety.

Embodiment B160. The oligomeric duplex of any one of embodiments B119-B159, wherein fewer than 50%, fewer than 40%, fewer than 30%, fewer than 20%, or fewer than 10% of the internucleoside linkages of the first modified oligonucleotide are modified internucleoside linkages.

Embodiment B161. The oligomeric duplex of any one of embodiments B119-B160, wherein the internucleoside linkages between the first and second nucleosides and the second and third nucleosides counting from the 5′ end of the first modified oligonucleotide, and the internucleoside linkages between the first and second nucleosides and the second and third nucleosides counting from the 3′ end of the first modified oligonucleotide are phosphorothioate internucleoside linkages.

Embodiment B162. The oligomeric duplex of any one of embodiments B119-B161, wherein the first modified oligonucleotide has an internucleoside linkage motif (5′ to 3′) selected from among: ssooooooooooooooooooss, ssooosooooooooooooooss, ssoososoooooooooooooss, and ssooooooooooooooooss; wherein each “o” represents a phosphodiester internucleoside linkage and each “s” represents a phosphorothioate internucleoside linkage.

Embodiment B163. The oligomeric duplex of any one of embodiments B119-B162, wherein fewer than 50%, fewer than 40%, fewer than 30%, fewer than 20%, or fewer than 10% of the internucleoside linkages of the second modified oligonucleotide are modified internucleoside linkages.

Embodiment B164. The oligomeric duplex of any one of embodiments B119-B163, wherein the internucleoside linkages between the first and second nucleosides and the second and third nucleosides counting from the 5′ end of the second modified oligonucleotide, and the internucleoside linkages between the first and second nucleosides and the second and third nucleosides counting from the 3′ end of the second modified oligonucleotide are phosphorothioate internucleoside linkages.

Embodiment B165. The oligomeric duplex of any one of embodiments B119-B164, wherein the second modified oligonucleotide has an internucleoside linkage motif (5′ to 3′) selected from among: ssooooooooooooooooss, ssooooooosooooooooss, ssoooooozozoooooooss, and ssooooooooooooooss; wherein each “o” represents a phosphodiester internucleoside linkage, each “s” represents a phosphorothioate internucleoside linkage, and each ‘z’ represents a mesyl phosphoramidate internucleoside linkage.

Embodiment B166. The oligomeric duplex of any one of embodiments B1-B165, wherein the first modified oligonucleotide has a first sugar motif (1^{st}) and the second modified oligonucleotide has a second sugar motif (2^{nd}) and the first and second sugar motifs are selected from among the following combinations (5′ to 3′): 1^{st}: efyydydyyyyyyfyfyyyyyee and 2^{nd}: eeyyyyfyfyfyyyyyyyyee; 1^{st}: efyydydyyyyyyfyfyyyyyyy and 2^{nd}: yyyyyyfyfyfyyyyyyyyyy; 1^{st}: efyydydyyyyyyfyfyyyyyee and 2^{nd}: yyyyyyfyfyfyyyyyyyyyy; 1^{st}: efyydydyyyyyyfyfyyyyyyy and 2^{nd}: eeyyyyfyfyfyyyyyyyyee; 1^{st}: efyyyyyyyyyyyfyfyyyyyee and 2^{nd}: eeyyyyyyyffyyyyyyyyee; 1^{st}: efyyyyyyyyyyyfyfyyyyyyy and 2^{nd}: yyyyyyyyyffyyyyyyyyyy; efyyyyyyyyyyyfyfyyyyyee and 2^{nd} yyy yffyy y 1^{st}: efyyyyyyyyyyyfyfyyyyyyy and 2^{nd}: eeyyyyyyyffyyyyyyyyee; 1^{st}: efyyydyyyyyyyfyfyyyyyee and 2^{nd}: eeyyyyyyyffyyyyyyyyee; 1^{st}: efyyydyyyyyyyfyfyyyyyyy and 2^{nd}: yyyyyyyyyffyyyyyyyyyy; 1^{st}: efyyydyyyyyyyfyfyyyyyee and 2^{nd}: yyyyyyyyffyyyyyyyyyy; 1^{st}: efyyydyyyyyyyfyfyyyyyyy and 2^{nd}: eeyyyyyyyffyyyyyyyyee; 1^{st}: efyydydyyyyyyfyfyyyyyee and 2^{nd}: eeyyyyyyyffyyyyyyyyee; 1^{st}: efyydydyyyyyyfyfyyyyyyy and 2^{nd}: yyyyyyyyyffyyyyyyyyyy; 1^{st}: efyydydyyyyyyfyfyyyyyee and 2^{nd}: yyyyyyyyyffyyyyyyyyyy; 1^{st}: efyydydyyyyyyfyfyyyyyyy and 2^{nd}: eeyyyyyyyffyyyyyyyyee; 1^{st}: efyyydyyyyyyyfyfyyyyyee and 2^{nd}: eeyyyyfyfyfyyyyyyyyee; 1^{st}: efyyydyyyyyyyfyfyyyyyyy and 2^{nd}: yyyyyyfyfyfyyyyyyyyyy; 1^{st}: efyyydyyyyyyyfyfyyyyyee and 2^{nd}: yyyyyyfyfyfyyyyyyyyyy; and 1^{st}: efyyydyyyyyyyfyfyyyyyyy and 2^{nd}: eeyyyyfyfyfyyyyyyyyee, wherein each “y” represents a 2′-OMe sugar moiety, each “e” represents a 2′-MOE sugar moiety, each “f” represents a 2′-fluoro sugar moiety, and each “d” represents a 2′-β-D-deoxyribosyl sugar moiety.

Embodiment B167. The oligomeric duplex of any one of embodiments B1-B165, wherein the first modified oligonucleotide has a first sugar motif (1^{st}) and the second modified oligonucleotide has a second sugar motif (2^{nd}) and the first and second sugar motifs are selected from among the following combinations (5′ to 3′): 1^{st}: efyydydyyyyyyfyfyyyyyee and 2^{nd} eeyyyyfyfffyyyyyyyyee; 1^{st}: efyydydyyyyyyfyfyyyyyyy and 2^{nd} yyyyyyfyfffyyyyyyyyyy; 1^{st}: efyydydyyyyyyfyfyyyyyee and 2^{nd} yyyyyyfyfffyyyyyyyyyy; 1^{st}: efyydydyyyyyyfyfyyyyyyy and 2^{nd}. eeyyyyfyfffyyyyyyyyee; 1^{St}. efyyyyyyyyyyyfyfyyyyyee and 2^{nd}. eeyyyyfyfffyyyyyyyyee; 1^{st}: efyyyyyyyyyyyfyfyyyyyyy and 2^{nd}. yyyyyyfyfffyyyyyyyyyy; 1^{st}: efyyyyyyyyyyyfyfyyyyyee and 2^{nd}: yyyyyfyfffyyyyyyyyyy; 1^{st}: efyyyyyyyyyyyfyfyyyyyyy and 2^{nd}. eeyyyyfyfffyyyyyyyyee; 1^{st}: efyyyyyyyyyyyfyfyyyyyee and 2^{nd}. eeyyyyyyyffyyyyyyyyee; 1^{st}: efyyyyyyyyyyyfyfyyyyyyy and 2^{nd} yyyyyyyyyffyyyyyyyyyy; 1^{st}: efyyyyyyyyyyyfyfyyyyyee and 2^{nd}. yyyyyyyyffyyyyyyyyyy; 1^{st}: efyyyyyyyyyyyfyfyyyyyyy and 2^{nd}. eeyyyyyyyffyyyyyyyyee; 1^{st}: efyyyyyyyyyyyfyfyyyyyee and 2^{nd}. eeyyyyfyfyfyyyyyyyyee; 1^{st}: efyyyyyyyyyyyfyfyyyyyyy and 2^{nd}. yyyyyyfyfyfyyyyyyyyyy; 1^{st}: efyyyyyyyyyyyfyfyyyyyee and 2^{nd}. yyyyyyfyfyfyyyyyyyyyy; 1^{st}: efyyyyyyyyyyyfyfyyyyyyy and 2^{nd}. eeyyyyfyfyfyyyyyyyyee; 11: efyyyfyyyyyyyfyfyyyyyyy and 2^{nd} yyyyyffyyyyyyyyyy; 1^{st}: efyyyfyyyyyyyfyfyyyyyee and 2^{nd} eeyyyyyyyffyyyyyyyyee; 1^{st}: efyyyfyyyyyyyfyfyyyyyyy and 2^{nd}. eeyyyyyffyyyyyyyyee; 1^{st}: efyyyfyyyyyyyfyfyyyyyee and 2^{nd}. yy yyffyyyyyyyyyy; 1^{st}: efyyyfyyyyyyyfyfyyyyyyy and 2^{nd}. yyyyyyfyfyfyyyyyyyyyy; 1^{st}: efyyyfyyyyyyyyfyyyyyee and 2^{nd}. eeyyyyfyfyfyyyyyyyyee; 1^{st}. efyyyfyyyyyyyfyfyyyyyyy and 2^{nd} eeyyyyfyfyfyyyyyyyyee; 1^{st}: efyyyfyyyyyyyfyfyyyyyee and 2^{nd} yyyyyyfyfyfyyyyyyyyyy; 1^{st}: efyyyfyyyyyyyfyfyyyee and 2^{nd}. eeyyyyfyfffyyyyyyce; 1^{st}: efyyyfyyyyyyyfyfyyyyy and 2^{nd}. yyyyyyfyfffyyyyyyyy; 1^{st}: efyyyfyyyyyyyfyfyyyee and 2^{nd}. yyyyyyfyfffyyyyyyyy; 1^{st}: efyyyfyyyyyyyfyfyyyyy and 2^{nd} eeyyyyfyfffyyyyyyee; 1^{st}: efyyyfyyyyyyyfyfyyyee and 2^{nd}. eeyyyyffyyyyyyyyee; 1^{st}: efyyyfyyyyyyyfyfyyyyy and 2^{nd}. yyyyyyffyyyyyyyyyy; 1^{st}: efyyyfyyyyyyyfyfyyyee and 2^{nd}. yyyyyffyyyyyyyyyY 1^{st}: efyyyfyyyyyyyfyfyyyyy and 2^{nd}. eeyyyyffyyyyyyyyee; 1^{st}: e[FHNA]yydydyyyyfyfyfyyyyyee and 2^{nd} eeyyyyfyfffyyyyyyyyee; 1′t: efyyyfyyyyyyyfyfyyyyyey and 2^{nd}. eeyyyyfyfffyyyyyyyyee; 1^{st}: efyyyfyyyyyyyfyfyyyyyey and 2^{nd}. yyyyyyfyfffyyyyyyyyyy; 1^{st}: e[FHNA]yydydyyyyfyfyfyyyyyyy and 2^{nd}. yyyyyyfyfffyyyyyyyyyy; 1^{st}: e[FHNA]yydydyyyyfyfyfyyyyyee and 2^{nd} yyyyyyfyfffyyyyyyyyyy; 1¹: e[FHNA]yydydyyyyfyfyfyyyyyyy and 2^{nd} eeyyyyfyfffyyyyyyyyee; 1^{st}: e[FHNA]yydydyyyyfyfyfyyyyyee and 2^{nd} eeyyyyfyfyfyyyyyyyyee; 1^{st}: e[FHNA]yydydyyyyfyfyfyyyyyyy and 2^{nd} yyyyyyfyfyfyyyyyyyyyy; 1^{st}: e[FHNA]yydydyyyyfyfyfyyyyyee and 2^{nd}: yyyyyyfyfyfyyyyyyyyyy; and 1^{st}: e[FHNA]yydydyyyyfyfyfyyyyyyy and 2^{nd}: eeyyyyfyfyfyyyyyyyyee, wherein each “y” represents a 2′-OMe sugar moiety, each “e” represents a 2′-MOE sugar moiety, each “f” represents a 2′-fluoro sugar moiety, each “[FHNA]” represents a 3′-fluoro-hexitol sugar moiety, and each “d” represents a 2′-D-D-deoxyribosyl sugar moiety.

Embodiment B168. The oligomeric duplex of any one of embodiments B1-B165, wherein the first modified oligonucleotide has a first sugar motif (1^{st}) and the second modified oligonucleotide has a second sugar motif (2^{nd}) and the first and second sugar motifs are selected from among the following combinations (5′ to 3′): 1^{st}: efyyyfyyyyyyyfyfyyyyyyy and 2^{nd} yyyyyyfyfffyyyyyyyyyy; 1^{st}: efyyyfyyyyyyyfyfyyyyyee and 2^{nd}. eeyyyyfyfffyyyyyyyyee; 1^{st}: efyyyfyyyyyyyfyfyyyyyee and 2^{nd}. yyyyyyfyfffyyyyyyyyyy; and 1^{st}: efyyyfyyyyyyyfyfyyyyyyy and 2^{nd}: eeyyyyfyfffyyyyyyyyee, wherein each “y” represents a 2′-OMe sugar moiety, each “e” represents a 2′-MOE sugar moiety, and each “f” represents a 2′-fluoro sugar moiety.

Embodiment B169. The oligomeric duplex of any one of embodiments B1-B165, wherein the first modified oligonucleotide has a first sugar motif (1^{st}) and the second modified oligonucleotide has a second sugar motif (2^{nd}) and the first and second sugar motifs are selected from among the following combinations (5′ to 3′): 1^{st}: efyyyfyyyyyyyfyfyyyyyyy and 2^{nd} yyyyyyyyyyyyyyyyy; 1^{st}: efyyyfyyyyyyyfyfyyyyyee and 2^{nd}. eeyyyyyyyyyyyyyyyyyee; 1^{st}: efyyyfyyyyyyyfyfyyyyyyy and 2^{nd}. eeyyyyyyyyyyyyyyyyyee; 1^{st}: efyyyfyyyyyyyfyfyyyyyee and 2^{nd}. yyyyyyyyyyyyyyyyyy; 1^{st}: efyyyfyyyyyyyfyfyyyyyyy and 2^{nd}: yyyyyyyyyydyyyyyyyyyy; 1^{st}: efyyyfyyyyyyyfyfyyyyyee and 2^{nd} eeyyyyyyyydyyyyyyyyee; 1^{st}: efyyyfyyyyyyyfyfyyyyyyy and 2^{nd}: eeyyyyyyyydyyyyyyyyee; 1^{st}: efyyyfyyyyyyyfyfyyyyyee and 2^{nd}: yyyyyyyyyydyyyyyyyyyy; 1^{st}: efyyyyyyyyyyyfyyyyyyyee and 2^{nd}. eeyyyyfyfffyyyyyyyyee; 1^{st}: efyyyyyyyyyyyfyyyyyyyyy and 2^{nd} yyyyyyfyfffyyyyyyyyyy; 1^{st}: efyyyyyyyyyyyfyyyyyyyee and 2^{nd}. yyyyyyfyfffyyyyyyyyyy; 1^{st}: efyyyyyyyyyyyfyyyyyyyyy and 2^{nd}. eeyyyyfyfffyyyyyyyyee; 1′t: efyyyyyyyyyyyfyyyyyyyee and 2^{nd}. eeyyyyfyfyfyyyyyyyyee; 1^{st}: efyyyyyyyyyyyfyyyyyyyyy and 2^{nd} yyyyyyfyfyfyyyyyyyyyy; 1^{st}: efyyyyyyyyyyyfyyyyyyyee and 2^{nd} yyyyyyfyfyfyyyyyyyyyy; 1^{st}: efyyyyyyyyyyyfyyyyyyyyy and 2^{nd} eeyyyyfyfyfyyyyyyce; 1^{st}: efyyydyyyyyyyfyfyyyyyee and 2^{nd} eeyyyyfyfffyyyyyyyyee; 1^{st}: efyyydyyyyyyyfyfyyyyyyy and 2^{nd} yyyyyyfyfffyyyyyyyyyy; 1^{st}: efyyydyyyyyyyfyfyyyyyee and 2^{nd}. yyyyyyfyfffyyyyyyyyyy; 1^{st}: efyyydyyyyyyyfyfyyyyyyy and 2^{nd} eeyyyyfyfffyyyyyyyyee; 1^{st}: efyyyfyyyyyyyfyfyyyee and 2^{nd} eeyyfyfffyyyyyyyyee; 1^{st}: efyyyfyyyyyyyfyfyyyyy and 2^{nd}. yyyyfyfffyyyyyyyyyy; 1^{st}: efyyyfyyyyyyyfyfyyyee and 2^{nd}. yyfyfffyyyyyyyyyy; 1^{st}: efyyyfyyyyyyyfyfyyyyy and 2^{nd}. eeyyfyfffyyyyyyyyee; 1^{st}: efyyyfyyyyyyyfydyyyyyee and 2^{nd} eeyyyfyfffyyyyyyyyee; 1^{st}: efyyyfyyyyyyyfydyyyyyyy and 2^{nd} yyyyyyfyfffyyyyyyyyyy; 1^{st}: efyyyfyyyyyyyfydyyyyyee and 2^{nd} yyyyyyfyfffyyyyyyyyyy; 1^{st}: efyyyfyyyyyyyfydyyyyyyy and 2^{nd}. eeyyyyfyfffyyyyyyyyee; 1^{st}: e[FHNA]yyyfyyyyyyyfyfyyyyyee and 2^{nd} eeyyyyfyfffyyyyyyyyee; 1^{st}: e[FHNA]yyyfyyyyyyyfyfyyyyyyy and 2^{nd} yyyyyyfyfffyyyyyyyyyy; 1^{st}: e[FHNA]yyyfyyyyyyyfyfyyyyyee and 2^{nd}: yyyyyyfyfffyyyyyyyyyy; and 1^{st}: e[FHNA]yyyfyyyyyyyfyfyyyyyyy and 2^{nd}: eeyyyyfyfffyyyyyyyyee, wherein each “y” represents a 2′-OMe sugar moiety, each “e” represents a 2′-MOE sugar moiety, each “f” represents a 2′-fluoro sugar moiety, each “[FHNA]” represents a 3′-fluoro-hexitol sugar moiety, and each “d” represents a 2′-D-D-deoxyribosyl sugar moiety.

Embodiment B170. The oligomeric duplex of any one of embodiments B1-B165, wherein the first modified oligonucleotide has a first sugar motif (1^{st}) and the second modified oligonucleotide has a second sugar motif (2^{nd}) and the first and second sugar motifs are selected from among the following combinations (5′ to 3′): 1^{st}: efyyyyyyyyyyyfyyyyyyyee and 2^{nd}: eeyy yyffyyyyyyyyee; 1^{st}: efyyyyyy fyyyyyyyy and 2^{nd}: yyyyyyyyyffyyyyyyyyyy; 1^{st}: efyyyyyyyyyfyyyyyyyee and 2^{nd}: yyyyyyyyffyyyyyyyyy; 1^{st}: efyyyyyyyyyyyfyyyyyyyyy and 2^{nd}. eeyyyyyyyffyyyyyyyyee; 1^{st}. efyyyfyyyyyyyfyyyyyyyee and 2^{nd}: eeyyyyfyfffyyyyyyyye; 1^{st}: efyyyfyyyyyyyfyyyyyyyyy and 2^{nd}: yyyyyyfyfffyyyyyyyyyy; 1^{st}: efyyyfyyyyyyyfyyyyyyyee and 2^{nd}: yyyyyyfyfffyyyyyyyyyy; 1^{st}: efyyyfyyyyyyyfyyyyyyyyy and 2^{nd}: eeyyyyfyfffyyyyyyyyee; 1^{st}: efyyyfyyyyyyyfyyyyyyyee and 2^{nd}: eeyyyyyyyffyyYyyyyyee; 1^{st}: efyyyfyyyyyyyfyyyyyyyyy and 2^{nd}: yyyy yyffy yyyyyy; 1^{st}: efyyyfyyyyyyyfyyyyyyyee and 2^{nd}: yyyyyyfyfffyyyyyyyyyy; and 1^{st}: efyyyfyyyyyyyfYyyyyyy and 2^{nd}: eeyyyyfyfffyyyyyyyyee, wherein each “y” represents a 2′-OMe sugar moiety, each “e” represents a 2′-MOE sugar moiety, and each “f” represents a 2′-fluoro sugar moiety.

Embodiment B171. The oligomeric duplex of any one of embodiments B119-B170, wherein the internucleoside linkages between the first and second nucleosides and the second and third nucleosides counting from the 5′ end of the first modified oligonucleotide, and the internucleoside linkages between the first and second nucleosides and the second and third nucleosides counting from the 3′ end of the first modified oligonucleotide are phosphorothioate internucleoside linkages and wherein all other internucleoside linkages of the first modified oligonucleotide are phosphodiester internucleoside linkages.

Embodiment B172. The oligomeric duplex of any one of embodiments B119-B171, wherein the internucleoside linkages between the first and second nucleosides and the second and third nucleosides counting from the 5′ end of the second modified oligonucleotide, and the internucleoside linkages between the first and second nucleosides and the second and third nucleosides counting from the 3′ end of the second modified oligonucleotide are phosphorothioate internucleoside linkages and wherein all other internucleoside linkages of the second modified oligonucleotide are phosphodiester internucleoside linkages.

Embodiment B173. The oligomeric duplex of any one of embodiments B119-B172, wherein the nucleobase sequence of the first modified oligonucleotide comprises or consists of the nucleobase sequence of any one of SEQ ID NOs: 967, 968, 971, 972, 974, 975, 1254, 1255, or 1033-1038.

Embodiment B174. The oligomeric duplex of any one of embodiments B1-B172, wherein a cytosine nucleobase in the first and/or second modified oligonucleotide is optionally 5-methylcytosine.

Embodiment B175. The oligomeric duplex of any one of embodiments B119-B174, wherein the duplex comprises a conjugate group comprising a conjugate moiety and a conjugate linker.

Embodiment B176. The oligomeric duplex of embodiment B175, wherein the conjugate moiety comprises an active drug substance, an aliphatic chain, a lipid, a peptide, a protein, a hydrocarbon, a polyamine, a polyamide, a polyether, a thioether, an aptamer, an antibody or antibody fragment, a vitamin, a fatty acid, a carbohydrate, an intercalator or a reporter molecule.

Embodiment B177. The oligomeric duplex of embodiment B175, wherein the conjugate group comprises a C22 alkyl, C20 alkyl, C17 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, or C5 alkyl, wherein the alkyl chain optionally has one or more unsaturated bonds.

Embodiment B178. The oligomeric duplex of embodiment B175, wherein the conjugate group comprises a 6-palmitamidohexyl moiety or a 2-(hydroxymethyl)-6-palmitamidohexyl moiety.

Embodiment B179. The oligomeric duplex of embodiment B175, wherein the duplex comprises a conjugate moiety that binds type 1 transferrin receptor (TfR1).

Embodiment B180. The oligomeric duplex of embodiment B179, wherein the conjugate moiety is selected from an antibody or fragment thereof, a protein or peptide, and an aptamer capable of binding TfR1.

Embodiment B181. The oligomeric duplex of embodiment B180, wherein the conjugate moiety is a cyclic protein or cyclic peptide.

Embodiment B182. The oligomeric duplex of embodiment B181, wherein the conjugate group consists of a bicycle ligand and a conjugate linker.

Embodiment B183. The oligomeric duplex of embodiment B182, wherein the bicycle ligand comprises a peptide consisting of 13-22 linked amino acids or amino acid mimetics and a molecular scaffold, wherein each of a first, a second, and a third amino acid of the peptide comprises a reactive group, each of which separately forms a bond with the molecular scaffold, thereby forming two peptide loops attached to the molecular scaffold.

Embodiment B184. The oligomeric duplex of embodiment B183 wherein the bicycle ligand comprises an amino acid sequence selected from any one of SEQ ID NO: 1071-1253.

Embodiment B185. The oligomeric duplex of any one of embodiments B182-B184, wherein the conjugate linker comprises a (bicyclo[6.1.0]nonyne)-formyl (BCN) moiety.

Embodiment B186. The oligomeric duplex of embodiment B185, wherein the conjugate linker comprises a 6-(BCN-carbamate)-2-(hydroxymethyl)hexyl phosphoryl moiety or a 6-(BCN-carbamate)hexyl phosphoryl moiety.

Embodiment B187. The oligomeric duplex of any one of embodiments B119-B174, comprising a conjugate group consisting of a conjugate moiety and a conjugate linker wherein the conjugate linker comprises a 6-(BCN-carbamate)-2-(hydroxymethyl)hexyl phosphoryl moiety or a 6-(BCN-carbamate)hexyl phosphoryl moiety.

Embodiment B188. The oligomeric duplex of any one of embodiments B119-B174, comprising a bicycle ligand comprising a peptide having an amino acid sequence selected from any one of SEQ ID NOs: 1071-1253 and wherein each cysteine forms a covalent bond with the molecular scaffold 1,1′,1″-(1,3,5-triazinane-1,3,5-triyl)tris(2-bromoethanone) (TATB) or the molecular scaffold is 1,1′,1″-(1,3,5-triazinane-1,3,5-triyl)triprop-2-en-1-one (TATA).

Embodiment B189. The oligomeric duplex of any one of embodiments B119-B174, or the oligomeric compound of any one of embodiments B198, B203, B208, B213, B218, B223, or B310, comprising a bicycle ligand having the following structure:

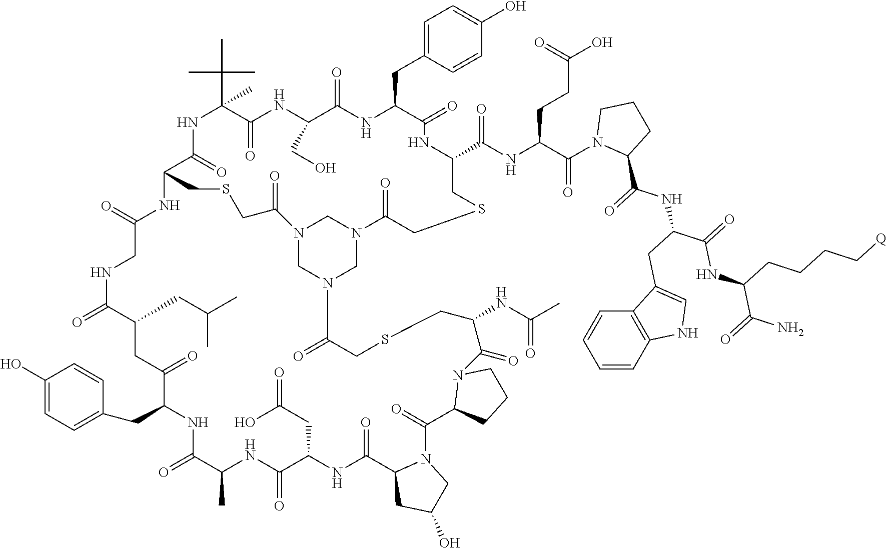

or a salt thereof, wherein Q is N₃ (BCY17901, SEQ ID NO: 1045), NH₂ (BCY21757, SEQ ID NO: 1202), SH (BCY21758, SEQ ID NO:1203), a conjugate linker, or a conjugate linker covalently connected to an oligonucleotide.

Embodiment B190. The oligomeric duplex of embodiment B189, wherein Q is N₃.

Embodiment B191. The oligomeric duplex of any one of embodiments B119-B174, or the oligomeric compound of any one of embodiments B198, 203, 208, 213, 218, 223, or 310, comprising a conjugate group having the following structure:

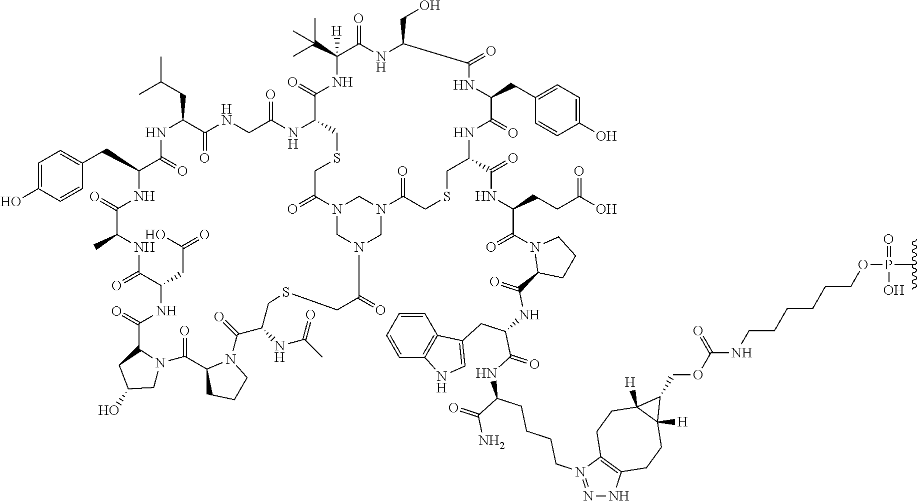

Embodiment B192. The oligomeric duplex of any one of embodiments B119-B174, or the oligomeric compound of any one of embodiments B198, B203, B208, B213, B218, B223, or B310, comprising a conjugate group having the following structure:

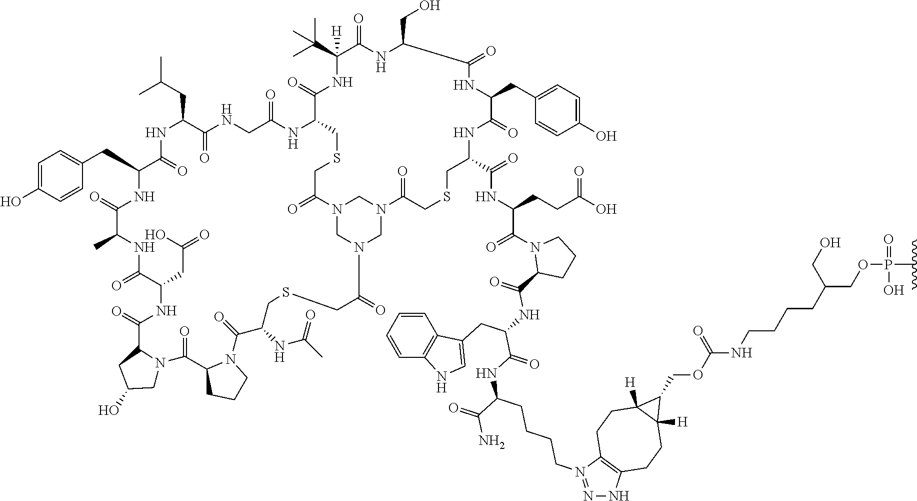

Embodiment B193. The oligomeric duplex of any one of embodiments B175-B192, wherein the bicycle ligand or conjugate group is attached to the second modified oligonucleotide.

Embodiment B194. The oligomeric duplex of any one of embodiments B175-B192, wherein the bicycle ligand or conjugate group is attached to the 5′- or 3′-end of the second modified oligonucleotide.

Embodiment B195. The oligomeric duplex of any one of embodiments B175-B192, or the oligomeric compound of any one of embodiments B198, B203, B208, B213, B218, B223, or B310, wherein the bicycle ligand or conjugate group is attached to the 5′-terminal nucleoside of the second modified oligonucleotide.

Embodiment B196. An oligomeric compound comprising a modified oligonucleotide according to the following chemical notation:

(SEQ ID NO: 1033)
vPTₑₛU_{fs}U_{yo}A_{yo}A_{yo}G_{do}A_{yo}U_{yo}G_{yo}A_{yo}G_{yo}A_{yo}C_{yo}A_{fo}G_{yo}A_{fo}
A_{yo}A_{yo}U_{yo}U_{yo}G_{ys}AₑₛAₑ,

wherein:
A=an adenine nucleobase,
C=a cytosine nucleobase,
G=a guanine nucleobase,
T=a thymine nucleobase,
U=a uracil nucleobase,
d=a 2′-β-D-deoxyribosyl sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate moiety.

Embodiment B197. An oligomeric compound comprising a modified oligonucleotide according to the following chemical notation:

(SEQ ID NO: 1027)
^{m}CₑₛAₑₛA_{yo}U_{yo}U_{yo}U_{yo}C_{yo}U_{yo}G_{yo}U_{fo}C_{fo}U_{yo}C_{yo}A_{yo}U_{yo}C_{yo}
U_{yo}U_{yo}A_{ys}AₑₛAₑ,

wherein:
A=an adenine nucleobase,
^{m}C=a 5-methyl cytosine nucleobase,
C=a cytosine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

Embodiment B198. An oligomeric compound comprising a conjugated modified oligonucleotide according to the following chemical notation: [X]ₙ-^{m}CₑₛAₑₛA_{yo}U_{yo}U_{yo}U_{yo}C_{yo}U_{yo}G_{yo}U_{fo}C_{fo}U_{yo}C_{yo}A_{yo}U_{yo}C_{yo}U_{yo}U_{yo}A_{ys}AₑₛAₑ-[X]ₖ (SEQ ID NO: 1039),

wherein:
^{m}C=a 5-methyl cytosine nucleobase,
C=a cytosine nucleobase,
A=an adenine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage,
[X]=a conjugate group comprising a bicycle ligand, and
n and k are 0 or 1, wherein if n=1 then k=0, and if n=0 then k=1.

Embodiment B199. An oligomeric duplex comprising the oligomeric compound according to embodiment B196 and the oligomeric compound according to embodiment B197.

Embodiment B200. An oligomeric duplex comprising the oligomeric compound according to embodiment B196 and the oligomeric compound according to embodiment B198.

Embodiment B201. An oligomeric compound comprising a modified oligonucleotide according to the following chemical notation: vP-TₑₛU_{fs}U_{yo}A_{yo}A_{do}G_{yo}A_{do}U_{yo}G_{yo}A_{yo}G_{yo}A_{yo}C_{yo}A_{fo}G_{yo}A_{fo}A_{yo}A_{yo}U_{yo}U_{yo}G_{ys}AₑₛAₑ (SEQ ID NO: 1034), wherein:
A=an adenine nucleobase,
C=a cytosine nucleobase,
G=a guanine nucleobase,
T=a thymine nucleobase,
U=a uracil nucleobase,
d=a 2′-β-D-deoxyribosyl sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate moiety.

Embodiment B202. An oligomeric compound comprising a modified oligonucleotide according to the following chemical notation: CₑₛAₑₛA_{yo}U_{yo}U_{yo}U_{yo}C_{fo}U_{yo}G_{fo}U_{yo}C_{fo}U_{yo}C_{yo}A_{yo}U_{yo}C_{yo}U_{yo}U_{yo}A_{ys}AₑₛAₑ (SEQ ID NO: 1028), wherein:
A=an adenine nucleobase,
C=a cytosine nucleobase,
G=a guanine nucleobase,
U=a uracil nucleobase,
e=a 2′-MOE sugar moiety,
f=a 2′-fluoro sugar moiety,
y=a 2′-Ome sugar moiety,
o=a phosphodiester internucleoside linkage, and
s=a phosphorothioate internucleoside linkage.

Embodiment B203. An oligomeric compound comprising a conjugated modified oligonucleotide according to the following chemical notation: [X]ₙ-CₑₛAₑₛA_{yo}U_{yo}U_{yo}U_{yo}C_{fo}U_{yo}G_{fo}U_{yo}C_{fo}U_{yo}C_{yo}A_{yo}U_{yo}C_{yo}U_{yo}U_{yo}A_{ys}AₑₛAₑ-[X]ₖ (SEQ ID NO: 1040), wherein:
C=a cytosine nucleobase,
A=an adenine nucleobase,
G=a guanine nucleobase,
U=a uracil nucleobase,
e=a 2′-MOE sugar moiety,
f=a 2′-fluoro sugar moiety,
y=a 2′-Ome sugar moiety,
o=a phosphodiester internucleoside linkage,
s=a phosphorothioate internucleoside linkage,
[X]=a conjugate group comprising a bicycle ligand, and
n and k are 0 or 1, wherein if n=1 then k=0, and if n=0 then k=1.

Embodiment B204. An oligomeric duplex comprising an oligomeric compound of embodiment B201 and an oligomeric compound of embodiment B202.

Embodiment B205. An oligomeric duplex comprising an oligomeric compound of embodiment B201 and an oligomeric compound of embodiment B203.

Embodiment B206. An oligomeric compound comprising a modified oligonucleotide according to the following chemical notation:

(SEQ ID NO: 1035)
vPTₑₛA_{fs}U_{yo}A_{yo}A_{yo}A_{do}U_{yo}A_{yo}G_{yo}A_{yo}U_{yo}U_{yo}C_{yo}U_{fo}G_{yo}U_{fo}
A_{yo}G_{yo}C_{yo}U_{yo}U_{ys}AₑₛAₑ,

wherein:
A=an adenine nucleobase,
C=a cytosine nucleobase,
G=a guanine nucleobase,
T=a thymine nucleobase,
U=a uracil nucleobase,
d=a 2′-β-D-deoxyribosyl sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate moiety.

Embodiment B207. An oligomeric compound comprising a modified oligonucleotide according to the following chemical notation: AₑₛAₑₛG_{yo}C_{yo}U_{yo}A_{yo}C_{yo}A_{yo}G_{yo}A_{fo}A_{fo}U_{yo}C_{yo}U_{yo}A_{yo}U_{yo}U_{yo}U_{yo}A_{ys}TₑₛAₑ (SEQ ID NO: 1029), wherein:
A=an adenine nucleobase,
C=a cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

Embodiment B208. An oligomeric compound comprising a conjugated modified oligonucleotide according to the following chemical notation: [X]ₙ-AₑₛAₑₛG_{yo}C_{yo}U_{yo}A_{yo}C_{yo}A_{yo}G_{yo}A_{fo}A_{fo}U_{yo}C_{yo}U_{yo}A_{yo}U_{yo}U_{yo}U_{yo}A_{ys}TₑₛAₑ-[X]ₖ (SEQ ID NO: 1041), wherein:
C=a cytosine nucleobase,
A=an adenine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage,
[X]=a conjugate group comprising a bicycle ligand, and
n and k are 0 or 1, wherein if n=1 then k=0, and if n=0 then k=1.

Embodiment B209. An oligomeric duplex comprising an oligomeric compound of embodiment B206 and an oligomeric compound of embodiment B207.

Embodiment B210. An oligomeric compound comprising an oligomeric compound of embodiment B206 and an oligomeric compound of embodiment B208.

Embodiment B211. An oligomeric compound comprising a modified oligonucleotide according to the following chemical notation:

(SEQ ID NO: 1036)
vPTₑₛA_{fs}U_{yo}A_{yo}A_{do}A_{yo}T_{do}A_{yo}G_{yo}A_{yo}U_{yo}U_{yo}C_{yo}U_{fo}G_{yo}U_{fo}
A_{yo}G_{yo}C_{yo}U_{yo}U_{ys}AₑₛAₑ,

wherein:
A=an adenine nucleobase,
C=a cytosine nucleobase,
G=a guanine nucleobase,
T=a thymine nucleobase,
U=a uracil nucleobase,
d=a 2′-β-D-deoxyribosyl sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate moiety.

Embodiment B212. An oligomeric compound comprising a modified oligonucleotide according to the following chemical notation: AₑₛAₑₛG_{yo}C_{yo}U_{yo}A_{yo}C_{fo}A_{yo}G_{fo}A_{yo}A_{fo}U_{yo}C_{yo}U_{yo}A_{yo}U_{yo}U_{yo}U_{yo}A_{ys}UₑₛAₑ (SEQ ID NO: 1030), wherein:
A=an adenine nucleobase,
C=a cytosine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

Embodiment B213. An oligomeric compound comprising a conjugated modified oligonucleotide according to the following chemical notation: [X]ₙ-AₑₛAₑₛG_{yo}C_{yo}U_{yo}A_{yo}C_{fo}A_{yo}G_{fo}A_{yo}A_{fo}U_{yo}C_{yo}U_{yo}A_{yo}U_{yo}U_{yo}U_{yo}A_{ys}UₑₛAₑ-[X]ₖ (SEQ ID NO: 1042), wherein:
C=a cytosine nucleobase,
A=an adenine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage,
[X]=a conjugate group comprising a bicycle ligand, and
n and k are 0 or 1, wherein if n=1 then k=0, and if n=0 then k=1.

Embodiment B214. An oligomeric duplex comprising an oligomeric compound of embodiment B211 and an oligomeric compound of embodiment B212.

Embodiment B215. An oligomeric duplex comprising an oligomeric compound of embodiment B211 and an oligomeric compound of embodiment B213.

Embodiment B216. An oligomeric compound comprising a modified oligonucleotide according to the following chemical notation:

(SEQ ID NO: 1037)
vPTₑₛU_{fs}A_{yo}A_{yo}G_{yo}T_{do}U_{yo}U_{yo}U_{yo}A_{yo}G_{yo}U_{yo}C_{yo}U_{fo}U_{yo}A_{fo}
A_{yo}U_{yo}C_{yo}U_{yo}U_{ys}AₑₛAₑ,

wherein:
A=an adenine nucleobase,
C=a cytosine nucleobase,
G=a guanine nucleobase,
T=a thymine nucleobase,
U=a uracil nucleobase,
d=a 2′-β-D-deoxyribosyl sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate moiety.

Embodiment B217. An oligomeric compound comprising a modified oligonucleotide according to the following chemical notation: AₑₛAₑₛG_{yo}A_{yo}U_{yo}U_{yo}A_{yo}A_{yo}G_{yo}A_{fo}C_{fo}U_{yo}A_{yo}A_{yo}A_{yo}A_{yo}C_{yo}U_{yo}U_{ys}AₑₛAₑ (SEQ ID NO: 1031), wherein:
A=an adenine nucleobase,
C=a cytosine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

Embodiment B218. An oligomeric compound comprising a conjugated modified oligonucleotide according to the following chemical notation: [X]ₙ-AₑₛAₑₛG_{yo}A_{yo}U_{yo}U_{yo}A_{yo}A_{yo}G_{yo}A_{fo}C_{fo}U_{yo}A_{yo}A_{yo}A_{yo}A_{yo}C_{yo}U_{yo}U_{ys}AₑₛAₑ-[X]ₖ (SEQ ID NO: 1043), wherein:
C=a cytosine nucleobase,
A=an adenine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage,
[X]=a conjugate group comprising a bicycle ligand, and
n and k are 0 or 1, wherein if n=1 then k=0, and if n=0 then k=1.

Embodiment B219. An oligomeric duplex comprising the modified oligonucleotide according to embodiment B216 and the modified oligonucleotide according to embodiment B217.

Embodiment B220. An oligomeric duplex comprising the modified oligonucleotide according to embodiment B216 and the conjugate according to embodiment B218.

Embodiment B221. An oligomeric compound comprising a modified oligonucleotide according to the following chemical notation:

(SEQ ID NO: 1038)
vPTₑₛU_{fs}A_{yo}A_{yo}G_{do}U_{yo}T_{do}U_{yo}U_{yo}A_{yo}G_{yo}U_{yo}C_{yo}U_{fo}U_{yo}A_{fo}
A_{yo}U_{yo}C_{yo}U_{yo}U_{ys}AₑsAₑ,

wherein:
A=an adenine nucleobase,
C=a cytosine nucleobase,
G=a guanine nucleobase,
T=a thymine nucleobase,
U=a uracil nucleobase,
d=a 2′-β-D-deoxyribosyl sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate moiety.

Embodiment B222. An oligomeric compound comprising a modified oligonucleotide according to the following chemical notation: AₑₛAₑₛG_{yo}A_{yo}U_{yo}U_{yo}A_{fo}A_{yo}G_{fo}A_{yo}C_{fo}U_{yo}A_{yo}A_{yo}A_{yo}A_{yo}C_{yo}U_{yo}U_{ys}AₑₛAₑ (SEQ ID NO: 1032), wherein:
A=an adenine nucleobase,
C=a cytosine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

Embodiment B223. An oligomeric compound comprising a conjugated modified oligonucleotide according to the following chemical notation: [X]ₙ-AₑₛAₑₛG_{yo}A_{yo}U_{yo}U_{yo}A_{fo}A_{yo}G_{fo}A_{yo}C_{fo}U_{yo}A_{yo}A_{yo}A_{yo}A_{yo}C_{yo}U_{yo}U_{ys}AₑₛAₑ-[X]ₖ (SEQ ID NO: 1044), wherein:
C=a cytosine nucleobase,
A=an adenine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage,
[X]=a conjugate group comprising a bicycle ligand, and
n and k are 0 or 1, wherein if n=1 then k=0, and if n=0 then k=1.

Embodiment B224. An oligomeric duplex comprising an oligomeric compound of embodiment B221 and an oligomeric compound of embodiment B222.

Embodiment B225. An oligomeric duplex comprising an oligomeric compound of embodiment B221 and an oligomeric compound of embodiment B223.

Embodiment B268. A population of oligomeric duplexes or oligomeric compounds of any one of embodiments B1-B225, wherein the population is enriched for first and/or second modified oligonucleotides comprising at least one particular phosphorothioate internucleoside linkage having a particular stereochemical configuration.

Embodiment B269. The population of embodiment B268, wherein the population is enriched for first and/or second modified oligonucleotides comprising at least one particular phosphorothioate internucleoside linkage having the (Sp) or (Rp) configuration.

Embodiment B270. The oligomeric duplex or oligomeric compound of any one of embodiments B1-B225, wherein the first modified oligonucleotide consists of 23 linked nucleosides and the second modified oligonucleotide consists of 21 linked nucleosides.

Embodiment B271. An antisense compound comprising or consisting of an oligomeric duplex or oligomeric compound of any one of embodiments B1-B225.

Embodiment B272. The antisense compound of embodiment B271, wherein the antisense compound is an RNAi agent capable of reducing the amount of PLN nucleic acid through the activation of RISC/Ago2.

Embodiment B273. A pharmaceutical composition comprising the oligomeric duplex or oligomeric compound of any one of embodiments B1-B225 or B270, the population of any one of embodiments B268-269, or the antisense compound of embodiment B271 or embodiment B272, and a pharmaceutically acceptable diluent or carrier.

Embodiment B274. The pharmaceutical composition of embodiment B273, wherein the pharmaceutically acceptable diluent is water or phosphate-buffered saline.

Embodiment B275. The pharmaceutical composition of embodiment B274, wherein the pharmaceutical composition consists essentially of the oligomeric duplex, oligomeric compound or the antisense compound, and water or phosphate-buffered saline.

Embodiment B276. A method of decreasing the amount of PLN RNA or PLN protein in a cell, tissue, organ or subject, comprising contacting the cell, tissue, organ or subject with the oligomeric duplex or oligomeric compound of any one of embodiments B1-B225 or B270, the population of any one of embodiments B268-B269, the antisense compound of embodiment B271 or embodiment B272, or the pharmaceutical composition of any one of embodiments B273-B275.

Embodiment B277. The method of embodiment B276, wherein the cell is a muscle cell and/or a cardiac cell.

Embodiment B278. A method comprising administering to a subject the oligomeric duplex or oligomeric compound of any one of embodiments B1-B225 or B270, the population of any one of embodiments B268-B269, the antisense compound of embodiment B271 or embodiment B272, or the pharmaceutical composition of any one of embodiments B273-B275; wherein the subject has or is at risk for developing a cardiovascular or cardiac injury, disease, condition or disorder, cardiomyopathy, cardiac arrythmia, and/or heart failure.

Embodiment B279. A method of preventing or treating a disease, disorder, condition or injury associated with cardiac calcium misregulation, or postponing a symptom of a disease, disorder or condition associated with a damaged, weakened and/or overworked heart, comprising administering to a subject having, or at risk of having, a disease, disorder or condition associated with cardiac calcium misregulation a therapeutically effective amount of the oligomeric duplex or oligomeric compound of any one of embodiments B1-B225 or B270, the population of any one of embodiments B268-B269, the antisense compound of embodiment B271 or embodiment B272, or the pharmaceutical composition of any one of embodiments B273-B275; wherein the disease, disorder, condition or injury is selected from a cardiac or cardiovascular disease, disorder, condition or injury, a cardiomyopathy, cardiac arrythmia, and/or heart failure.

Embodiment B280. A method of preventing or treating a cardiac or cardiovascular disease, disorder, condition or injury associated with heart failure, or postponing a symptom of heart failure, comprising administering to a subject having, or at risk of having, a cardiac or cardiovascular disease, disorder, condition or injury a therapeutically effective amount of the oligomeric duplex or oligomeric compound of any one of embodiments B1-B225 or B270, the population of any one of embodiments B268-B269, the antisense compound of embodiment B271 or embodiment B272, or the pharmaceutical composition of any one of embodiments B273-B275; wherein the disease, disorder, condition or injury is a cardiomyopathy, cardiac arrythmia, and/or heart failure.

Embodiment B281. A method of decreasing the amount of PLN RNA and/or PLN protein in the heart of a subject having or at risk of developing a disease, disorder or condition associated with a damaged, weakened and/or overworked heart, comprising administering to a subject having, or at risk of having, a disease, disorder or condition associated with a damaged, weakened and/or overworked heart a therapeutically effective amount of the oligomeric duplex or oligomeric compound of any one of embodiments B1-B225 or B270, the population of any one of embodiments B268-B269, the antisense compound of embodiment B271 or embodiment B272, or the pharmaceutical composition of any one of embodiments B273-B275; wherein the disease, disorder, condition or injury is a cardiomyopathy, cardiac arrythmia, and/or heart failure.

Embodiment B282. The method of any one of embodiments B276-B281, wherein the amount of PLN RNA and/or PLN protein in cardiac muscle of the subject is decreased.

Embodiment B283. The method of any one of embodiments B276-B281, wherein the disease, disorder or condition is cardiomyopathy or arrhythmia.

Embodiment B284. The method of embodiment B283, wherein the cardiomyopathy is genetic cardiomyopathy.

Embodiment B285. The method of embodiment B284, wherein the genetic cardiomyopathy is associated with PLN p.Arg14del, Arg9Cys (R9C), or Arg25Cys (R25C) genetic mutations.

Embodiment B286. The method of embodiment B283, wherein the cardiomyopathy is dilated cardiomyopathy (DCM).

Embodiment B287. The method of embodiment B286 wherein the DCM is genetic DCM.

Embodiment B288. The method of embodiment B287, wherein the genetic DCM is associated with TTN, LMNA, RBM20, SCN5A, MYH7, TNNT2, and TPM1 mutations.

Embodiment B289. The method of embodiment B286, wherein the DCM is arrhythmogenic DCM.

Embodiment B290. The method of any one of embodiments B278-B282, wherein the heart failure is heart failure with preserved ejection fraction (HFpEF), heart failure with reduced ejection fraction (HFrEF), acute heart failure, or worsening of chronic heart failure.

Embodiment B291. The method of embodiment B283, wherein the arrhythmia is ventricular tachycardia (vtac) or ventricular fibrillation (vfib).

Embodiment B292. The method of embodiment B279, wherein the symptom of a disease, disorder or condition associated with a damaged, weakened and/or overworked heart, or heart failure is reduced myocardial contractile function and/or impaired relaxation of the heart.

Embodiment B293. The method of any one of embodiments B278-B292, wherein the method prevents or slows progression of damage, weakening and/or overworked heart effects and/or heart failure.

Embodiment B294. The method of any one of embodiments B278-B292, wherein administering of the oligomeric duplex or oligomeric compound of any one of embodiments B1-B225 or B270, the population of any one of embodiments B268-B269, the antisense compound of embodiment B271 or embodiment B272, or the pharmaceutical composition of any one of embodiments B273-B275 improves cardiac function, cardiovascular death, cardiac dilation, cardiac fibrosis, low voltage ECG, diastolic calcium uptake, ejection fraction (EF), left ventricular ejection fraction (LVEF), left ventricular end systolic volume (LVESV), left ventricular end diastolic volume (LVEDV), mitral valve flow profile, left ventricle (LV) strain, left ventricle (LV) strain rate, infarct size, heart failure hospitalization, 6 minute walk test (6MWT), the Kansas City Cardiomyopathy Questionnaire Score (KCCQS), heart rate, or heart rhythm in the subject.

Embodiment B295. The method of any one of embodiments B276-B294, wherein the oligomeric duplex or oligomeric compound of any one of embodiments B1-B225 or B270, the population of any one of embodiments B268-B269, the antisense compound of embodiment B271 or embodiment B272, or the pharmaceutical composition of any one of embodiments B273-B275 comprises a conjugate moiety having affinity for a transferrin receptor.

Embodiment B296. The method any one of embodiments B276-B294, wherein the oligomeric duplex or oligomeric compound of any one of embodiments B1-B225 or B270, the population of any one of embodiments B268-B269, the antisense compound of embodiment B271 or embodiment B272, or the pharmaceutical composition of any one of embodiments B273-B275 comprises a bicycle ligand having affinity for a transferrin receptor.

Embodiment B297. Use of the oligomeric duplex or oligomeric compound of any one of embodiments B1-B225 or B270, the population of any one of embodiments B268-B269, the antisense compound of embodiment B271 or embodiment B272, or the pharmaceutical composition of any one of embodiments B273-B275 for treating or preventing a disease, disorder or condition associated with a damaged, weakened and/or overworked heart or heart failure.

Embodiment B298. Use of the oligomeric duplex or oligomeric compound of any one of embodiments B1-B225 or B270, the population of any one of embodiments B268-269, the antisense compound of embodiment B271 or embodiment B272, or the pharmaceutical composition of any one of embodiments B273-B275 in the manufacture of a medicament for treating or preventing a disease, disorder or condition associated with a damaged, weakened and/or overworked heart or heart failure.

Embodiment B299. The use of embodiment B297 or B298, wherein the disease, disorder or condition associated with a damaged, weakened and/or overworked heart or associated with heart failure is cardiomyopathy, cardiac arrythmia, and/or heart failure.

Embodiment B300. The use of embodiment B299, wherein the cardiomyopathy is genetic cardiomyopathy.

Embodiment B301. The use of embodiment B300, wherein the genetic cardiomyopathy is associated with PLN p.Arg14del, Arg9Cys (R9C), or Arg25Cys (R25C) genetic mutations.

Embodiment B302. The use of embodiment B299, wherein the cardiomyopathy is dilated cardiomyopathy (DCM).

Embodiment B303. The use of embodiment B302, wherein the DCM is genetic DCM.

Embodiment B304. The use of embodiment B303, wherein the genetic DCM is associated with TTN, LMNA, RBM20, SCN5A, MYH7, TNNT2, and TPM1 mutations.

Embodiment B305. The use of embodiment B302, wherein the DCM is arrhythmogenic DCM.

Embodiment B306. The use of embodiment B299, wherein the heart failure is heart failure with preserved ejection fraction (HFpEF), heart failure with reduced ejection fraction (HFrEF), acute heart failure, or worsening of chronic heart failure.

Embodiment B307. The use of embodiment B299, wherein the arrhythmia is ventricular tachycardia (vtac) or ventricular fibrillation (vfib).

Embodiment B308. An oligomeric compound comprising a modified oligonucleotide according to the following chemical notation:

(SEQ ID NO: 1255)
vPTₑₛA_{fs}U_{yo}A_{yo}A_{yo}A_{yo}U_{yo}A_{yo}G_{yo}A_{yo}U_{yo}U_{yo}C_{yo}U_{fo}G_{yo}U_{fo}
A_{yo}G_{yo}C_{yo}U_{yo}U_{ys}AₑₛAₑ,

wherein:
A=an adenine nucleobase,
C=a cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate moiety.

Embodiment B309. An oligomeric compound comprising a modified oligonucleotide according to the following chemical notation: AₑₛAₑₛG_{yo}C_{yo}U_{yo}A_{yo}C_{yo}A_{yo}G_{yo}A_{fo}A_{fo}U_{yo}C_{yo}U_{yo}A_{yo}U_{yo}U_{yo}U_{yo}A_{ys}TₑₛAₑ (SEQ ID NO: 1029), wherein:
A=an adenine nucleobase,
C=a cytosine nucleobase,
G=a guanine nucleobase,
U=a uracil nucleobase,
T=a thymine nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

Embodiment B310. An oligomeric compound comprising a conjugated modified oligonucleotide according to the following chemical notation: [X]ₙ-AₑₛAₑₛG_{yo}C_{yo}U_{yo}A_{yo}C_{yo}A_{yo}G_{yo}A_{fo}A_{fo}U_{yo}C_{yo}U_{yo}A_{yo}U_{yo}U_{yo}U_{yo}A_{ys}TₑₛAₑ-[X]ₖ (SEQ ID NO: 1041), wherein:
C=a cytosine nucleobase,
A=an adenine nucleobase,
G=a guanine nucleobase,
U=a uracil nucleobase,
T=a thymine nucleobase,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage,
[X]=a conjugate group comprising a bicycle ligand, and
n and k are 0 or 1, wherein if n=1 then k=0, and if n=0 then k=1.

Embodiment B311. An oligomeric duplex comprising the oligomeric compound according to embodiment B308 and the oligomeric compound according to embodiment B309.

Embodiment B312. An oligomeric duplex comprising the oligomeric compound according to embodiment B308 and the oligomeric compound according to embodiment B310.

### I. Oligionucleotides

In certain embodiments, provided herein are oligomeric compounds and oligomeric duplexes comprising a modified antisense oligonucleotide complementary to PLN RNA and a modified sense oligonucleotide complementary to an antisense oligonucleotide. Also provided herein in certain embodiments are oligomeric compounds comprising or consisting of a modified oligonucleotide, e.g., a modified antisense oligonucleotide complementary to PLN RNA or a modified sense oligonucleotide complementary to an antisense oligonucleotide that is complementary to PLN RNA. Modified antisense and/or sense oligonucleotides comprise at least one modified nucleoside (comprising a modified sugar moiety or sugar surrogate and/or a modified nucleobase) and/or at least one modified internucleoside linkage. Examples of certain modified nucleosides and modified internucleoside linkages suitable for use in modified antisense and/or sense oligonucleotides are described herein.

### A. Modified Nucleosides

Modified nucleosides comprise a modified sugar moiety or sugar surrogate or a modified nucleobase or both a modified sugar moiety (or sugar surrogate) and a modified nucleobase. In certain embodiments, modified nucleosides comprising the following modified sugar moieties or sugar surrogates and/or the following modified nucleobases may be incorporated into modified antisense and/or sense oligonucleotides of the invention.

### 1. Modified Sugar Moieties and Sugar Surrogates

In certain embodiments, modified sugar moieties are non-bicyclic modified sugar moieties. In certain embodiments, modified sugar moieties are bicyclic or tricyclic sugar moieties. In certain embodiments, a modified sugar moiety is a modified ribosyl sugar moiety. In some embodiments, a modified sugar moiety is a 2′-deoxyfuranosyl sugar moiety, e.g., a 2′-deoxy sugar moiety.

In certain embodiments, modified sugar moieties are non-bicyclic modified furanosyl sugar moieties comprising one or more substituent groups including, but not limited to, substituents at the 2′, 3′, 4′, and/or 5′ positions. In certain embodiments, the furanosyl sugar moiety is a ribosyl sugar moiety. In certain embodiments one or more non-bridging substituent of non-bicyclic modified sugar moieties is branched.

In certain embodiments, non-bicyclic modified sugar moieties comprise a substituent group at the 2′-position. Examples of substituent groups suitable for the 2′-position of modified sugar moieties include but are not limited to: 2′-F, 2′-OCH₃ (“OMe” or “O-methyl”), and 2′-O(CH₂)₂OCH₃ (“MOE” or “O-methoxyethyl”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and R˜ is, independently, H, an amino protecting group, or substituted or unsubstituted C₁-C₁₀ alkyl, —O(CH₂)₂ON(CH₃)₂(“DMAOE”), or 2′-O(CH₂)₂O(CH₂)₂N(CH₃)₂(“DMAEOE”). Synthetic methods for some of these 2′-substituent groups can be found, e.g., in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl.

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂₀N(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted sugar moiety of a modified nucleoside comprises a non-bridging 2′-substituent group selected from: F, OCF₃, OCH₃, OCH₂CH₂OCH₃, O(CH₂)₂SCH₃, O(CH₂)₂₀N(CH₃)₂, O(CH₂)₂O(CH₂)₂N(CH₃)₂, O(CH₂)₂₀N(CH₃)₂(“DMAOE”), O(CH₂)₂O(CH₂)₂N(CH₃)₂ (“DMAEOE”), and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂₀N(CH₃)₂, O(CH₂)₂O(CH₂)₂N(CH₃)₂, and OCH₂C(═O)—N(H)CH₃ (“NMA”).

In certain embodiments, a 2′-substituted sugar moiety of a modified nucleoside comprises a 2′-substituent group selected from: F, OCH₃, and OCH₂CH₂OCH₃.

In certain embodiments, modified furanosyl sugar moieties and nucleosides incorporating such modified furanosyl sugar moieties are further defined by isomeric configuration. For example, a 2′-deoxyfuranosyl sugar moiety (i.e., 2′-(H)H furanosyl sugar moiety) may be in seven isomeric configurations other than the naturally occurring D-D-deoxyribosyl configuration. Such modified sugar moieties are described in, e.g., WO2020/072991, incorporated by reference herein. A 2′-modified sugar moiety has an additional stereocenter at the 2′-position relative to a 2′-deoxyfuranosyl sugar moiety; therefore, such sugar moieties have a total of sixteen possible isomeric configurations. Modified furanosyl sugar moieties described herein are in the β-D-ribosyl isomeric configuration unless otherwise specified.

In certain embodiments, non-bicyclic modified sugar moieties comprise a substituent group at the 4′-position. Examples of substituent groups suitable for the 4′-position of modified sugar moieties include, but are not limited to, alkoxy (e.g., methoxy), alkyl, and those described in Manoharan et al., WO 2015/106128.

In certain embodiments, non-bicyclic modified sugar moieties comprise a substituent group at the 3′-position. Examples of substituent groups suitable for the 3′-position of modified sugar moieties include, but are not limited to, alkoxy (e.g., methoxy), alkyl (e.g., methyl, ethyl).

In certain embodiments, non-bicyclic modified sugar moieties comprise a substituent group at the 5′-position. Examples of substituent groups suitable for the 5′-position of modified sugar moieties include, but are not limited to, allyl (e.g., vinyl), alkoxy (e.g., methoxy), alkynyl, and alkyl (e.g., methyl (R or S), ethyl (R or S)).

In certain embodiments, non-bicyclic modified sugar moieties comprise more than one non-bridging sugar substituent, for example, 2′-F-5′-methyl sugar moieties, such as described in Migawa et al., US2010/0190837, or alternative 2′- and 5′-modified sugar moieties as described in Rajeev et al., US2013/0203836.

In naturally occurring nucleic acids, sugars are linked to one another 3′ to 5′. In certain embodiments, oligonucleotides include one or more nucleoside or sugar moiety linked at an alternative position, for example at the 2′ or inverted 5′ to 3′. For example, where the linkage is at the 2′ position, the 2′-substituent groups may instead be at the 3′-position. As used herein, “inverted nucleoside” means a nucleotide having a non-natural linkage, e.g., a 3′ to 3′ and/or 5′ to 5′ internucleoside linkage, as shown herein. An “inverted sugar moiety” means the sugar moiety of an inverted nucleoside or an abasic sugar moiety, e.g., having a 3′ to 3′ and/or 5′ to 5′ internucleoside linkage.

Certain modified sugar moieties comprise a substituent that bridges two atoms of the furanosyl ring to form a second ring, resulting in a bicyclic sugar moiety. In certain embodiments, the bicyclic sugar moiety comprises a bridge between the 4′ and the 2′ furanose ring atoms. Examples of such 4′ to 2′ bridging sugar substituents include, but are not limited to: 4′-CH₂-2′, 4′—(CH₂)₂-2′, 4′—(CH₂)₃-2′, 4′—CH₂—O-2′ (“LNA”), 4′-CH₂—S-2′, 4′—(CH₂)₂—O-2′ (“ENA”), 4′-CH(CH₃)—O-2′ (referred to as “constrained ethyl” or “cEt” when in the S configuration), 4′-CH₂—O—CH₂-2′, 4′—CH₂—N(R)-2′, 4′—CH(CH₂OCH₃)—O-2′ (“constrained MOE” or “cMOE”) and analogs thereof, 4′-C(CH₃)(CH₃)—O-2′ and analogs thereof, 4′-CH₂—N(OCH₃)-2′ and analogs thereof, 4′-CH₂—O—N(CH₃)-2′, 4′—CH₂—C(H)(CH₃)-2′,4′—CH₂—C(═CH₂)-2′ and analogs thereof, 4′-C(RₐR_{b})—N(R)—O-2′, 4′—C(RₐR_{b})—O—N(R)-2′, 4′—CH₂—O—N(R)-2′, and 4′-CH₂—N(R)—O-2′, wherein each R, Rₐ, and R_{b} is, independently, H, a protecting group, or C₁-C₁₂ alkyl. Representative U.S. patents that teach the preparation of such bicyclic sugar moieties include, but are not limited to: Imanishi et al., U.S. Pat. No. 7,427,672; Swayze et al., U.S. Pat. No. 7,741,457, and Swayze et al., U.S. Pat. No. 8,022,193; Seth et al., U.S. Pat. No. 8,278,283; Prakash et al., U.S. Pat. No. 8,278,425; Seth et al., U.S. Pat. No. 8,278,426.

In certain embodiments, such 4′ to 2′ bridges independently comprise from 1 to 4 linked groups independently selected from: —[C(Ra)(Rb)]n-, —[C(Ra)(Rb)]n-O—, C(Ra)═C(Rb)—, C(Ra)=N—, C(=NRa)—, —C(═O)—, —C(═S)—, —O—, —Si(Ra)₂—, —S(═O)x-, and N(Ra)—; wherein:
x is 0, 1, or 2;
n is 1, 2, 3, or 4;
   each Ra and Rb is, independently, H, a protecting group, hydroxyl, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, heterocycle radical, substituted heterocycle radical, heteroaryl, substituted heteroaryl, C5-C7 alicyclic radical, substituted C5-C7 alicyclic radical, halogen, OJI, NJ1J2, SJ1, N3, COOJ1, acyl (C(═O)—H), substituted acyl, CN, sulfonyl (S(═O)₂-J1), or sulfoxyl (S(═O)-J1); and each J1 and J2 is, independently, H, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, acyl (C(═O)—H), substituted acyl, a heterocycle radical, a substituted heterocycle radical, C1-C12 aminoalkyl, substituted C1-C12 aminoalkyl, or a protecting group.

Additional bicyclic sugar moieties are known in the art, see, for example: Wan, et al., J. Medicinal Chemistry, 2016, 59, 9645-9667; Wengel et al., U.S. Pat. No. 8,080,644; Ramasamy et al., U.S. Pat. No. 6,525,191; Seth et al., U.S. Pat. No. 7,547,684; and Seth et al., U.S. Pat. No. 7,666,854.

In certain embodiments, bicyclic sugar moieties and nucleosides incorporating such bicyclic sugar moieties are further defined by isomeric configuration. For example, an LNA nucleoside (described herein) may be in the α-L configuration or in the β3-D configuration.

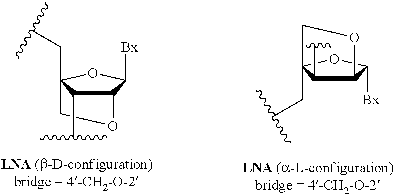

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). The addition of locked nucleic acids to siRNAs has been shown to increase siRNA stability in serum, and to reduce off-target effects (Elmen, J. et al., (2005) Nucleic Acids Research 33(1):439-447; Mook, OR. et al., (2007) Mol Canc Ther 6(3):833-843; Grunweller, A. et al., (2003) Nucleic Acids Research 31(12):3185-3193). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, a modified nucleoside comprises a sugar surrogate. In certain such embodiments, the oxygen atom of the sugar moiety is replaced, e.g., with a sulfur, carbon or nitrogen atom. In certain such embodiments, such sugar surrogates also comprise bridging and/or non-bridging substituents as described herein. For example, certain sugar surrogates comprise a 4′-sulfur atom and a substitution at the 2′-position and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), altritol nucleic acid (“ANA”), manitol nucleic acid (“MNA”), fluoro HNA:

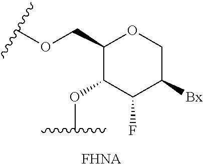

(“FHNA”, see e.g., Egli, et. al., J Am Chem Soc (2011) 133(41):16642-16649, Swayze et al., U.S. Pat. No. 8,088,904; and Swayze et al., U.S. Pat. No. 8,440,803); FHNA can also be referred to as a F-THP or 3′-fluoro tetrahydropyran or 3′-FHNA), and nucleosides comprising additional modified THP compounds having the formula:

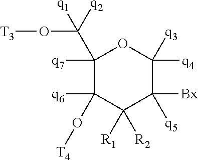

wherein, independently, for each of said modified THP nucleoside:
Bx is a nucleobase moiety;
T₃ and T₄ are each, independently, an internucleoside linkage, linking the modified THP nucleoside to the remainder of an oligonucleotide or one of T₃ and T₄ is an internucleoside linkage, linking the modified THP nucleoside to the remainder of an oligonucleotide and the other of T₃ and T₄ is H, a hydroxyl protecting group, a linked conjugate group, or a 5′ or 3′-terminal group;
q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and
each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is 0, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported. As used here, the term “morpholino” means a sugar surrogate having the following structure:

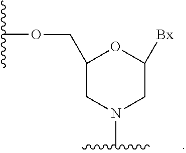

In certain embodiments, a morpholino is modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid, and nucleosides and oligonucleotides described in Manoharan et al., U.S. Pat. No. 10,913,767. Representative U.S. patents that teach the preparation of PNA compounds include, but are not limited to, U.S. Pat. Nos. 5,539,082; 5,714,331; and 5,719,262.

In certain embodiments, sugar surrogates are the “unlocked” sugar structure of UNA (unlocked nucleic acid) nucleosides. UNA is an unlocked acyclic nucleic acid, wherein any of the bonds of the sugar has been removed, forming an unlocked sugar surrogate. Representative U.S. publications that teach the preparation of UNA include, but are not limited to, US Patent Publication No. 2011/0313020.

In certain embodiments, sugar surrogates are the glycerol as found in GNA (glycol nucleic acid) nucleosides as depicted below:

### (S)-GNA

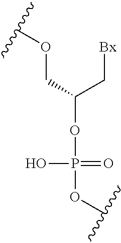

where Bx represents any nucleobase.

Many other bicyclic and tricyclic sugar and sugar surrogates are known in the art that can be used in modified nucleosides. In certain embodiments, the modified oligonucleotide comprises a modification disclosed in U.S. Pat. No. 10,233,448 or 11,504,391.

### 2. Modified Nucleobases

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside. In certain embodiments, modified oligonucleotides contain no abasic nucleosides. In certain embodiments, modified oligonucleotides comprise one or more inosine nucleosides (i.e., nucleosides comprising a hypoxanthine nucleobase). An “unmodified nucleobase” is unmodified adenine (A), unmodified thymine (T), unmodified cytosine (C), unmodified uracil (U), or unmodified guanine (G). A modified nucleobase is a group of atoms other than unmodified A, T, C, U, or G capable of pairing with at least one other nucleobase. 5-methylcytosine and hypoxanthine are examples of modified nucleobases.

Unless otherwise indicated, modified adenine has structure (I):

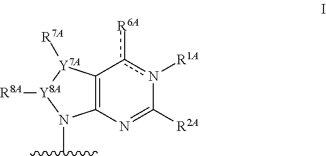

wherein: R^{1A} is absent or H; R^{2A} is H, C₁-C₆ alkyl, substituted C₁-C₆ alkyl, C₁-C₆ thioalkyl, or substituted C₁-C₆ thioalkyl, C₁-C₆ alkyloxy, or substituted C₁-C₆ alkyloxy; R^{6A} is H, N(R^{a})(R^{b}), oxo, acetyl, formyl, or O-phenyl; Y^{7A} is N and R^{7A} is absent or is C₁-C₆ alkyl; or Y^{7A} is C and R^{7A} is selected from H, C₁-C₆ alkyl, or N(R^{a})(R^{b}); Y^{8A} is N and R^{8A} is absent, or Y^{8A} is C and R^{8A} is selected from H, a halogen, OH, C₁-C₆ alkyl, or substituted C₁-C₆ alkyl; R^{a} and R^{b} are independently selected from H, C₁-C₆ alkyl, substituted C₁-C₆ alkyl, C₁-C₆ alkenyl, substituted C₁-C₆ alkenyl, acetyl, or formyl, or together form a 5-7-membered heterocycle; excluding where Y^{7A} is N and R^{7A} is absent; Y^{8A} is C, R^{8A} is H, R^{2A} is H, and R^{6A} is NH₂ (unmodified adenine).

Hypoxanthine has structure (Ia):

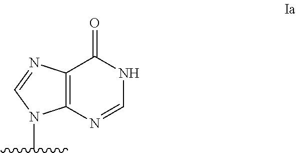 [SMILES: *n1cnc2c(=O)[nH]cnc21]

Hypoxanthine is considered a modified adenine, where Y^{7A} is N and R^{7A} is absent; Y^{8A} is C, R^{8A} is H, R^{1A} is H, R^{2A} is H, and R^{6A} is oxo.

Unless otherwise indicated, modified guanine has structure (II):

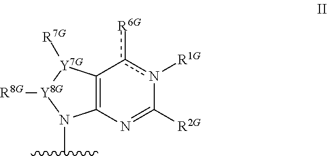

wherein: R^{2G} is N(R^{a})(R^{b}); R^{6G} is oxo and R^{IG} is H, or R^{6G} is selected from O—C₁-C₆ alkyl or S—C₁-C₆ alkyl and R^{IG} is absent; Y^{7G} is N and R^{7G} is absent or is C₁-C₆ alkyl; or Y^{7G} is C and R^{7G} is selected from H, C₁-C₆ alkyl, or N(R^{a})(R^{b}); Y^{8G} is N and R^{8G} is absent, or Y^{8G} is C and R^{8G} is selected from H, a halogen, OH, C₁-C₆ alkyl, or substituted C₁-C₆ alkyl; R and R^{b} are independently selected from H, C₁-C₆ alkyl, substituted C₁-C₆ alkyl, C₁-C₆ alkenyl, substituted C₁-C₆ alkenyl, acetyl, or formyl, or together form a 5-7-membered heterocycle; excluding where Y^{7G} is N and R^{7G} is absent; Y^{8G} is C, R^{8G} is H, R^{2G} is NH₂, and R^{6G} is ═O, and R^{1G} is H (unmodified guanosine).

Unless otherwise indicated, modified thymine or modified uracil has structure (III):

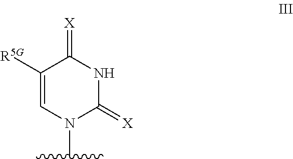

wherein: each X is independently 0 or S and R^{5U} is selected from H, OH, halogen, O—C₁-C₂₀ alkyl, 0-C₁-C₁₂ substituted alkyl, C₁-C₁₂ alkyl, substituted C₁-C₁₂ alkyl, C₁-C₁₂ alkenyl, substituted C₁-C₁₂ alkenyl, C₁-C₁₂ alkynyl, substituted C₁-C₁₂ alkynyl; wherein if each X is 0, R^{5U} is not H or CH₃ (unmodified uracil and unmodified thymine, respectively).

Unless otherwise indicated, modified cytosine has structure (IV):

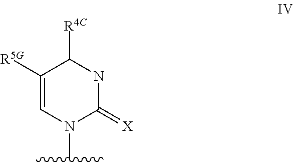

wherein: X is selected from O or S, R^{4C} is N(R^{a})(R^{b}); R^{5C} is selected from H, OH, halogen, O—C₁-C₁₂ alkyl, O—C₁-C₁₂ substituted alkyl, C₁-C₁₂ alkyl, substituted C₁-C₁₂ alkyl, C₁-C₁₂ alkenyl, substituted C₁-C₁₂ alkenyl; R and R^{b} are independently selected from H, C₁-C₆ alkyl, substituted C₁-C₆ alkyl, C₁-C₆ alkenyl, substituted C₁-C₆ alkenyl, C₁-C₁₂ alkynyl, substituted C₁-C₁₂ alkynyl; acetyl, or formyl, or together form a 5-7-membered heterocycle; excluding where X is O, R^{4C} is NH₂ and R^{5C} is H (unmodified cytosine).

In certain embodiments, modified nucleobases of a modified oligonucleotide are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 5-methylcytosine, 1-methylpsuedouridine, 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C═C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo (particularly 5-bromo), 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases, as well as other modified nucleobases include without limitation, Rogers et al., U.S. Pat. No. 5,134,066; Benner et al., U.S. Pat. No. 5,432,272; Matteucci et al., U.S. Pat. No. 5,502,177; Froehler et al., U.S. Pat. No. 5,594,121; and Cook et al., U.S. Pat. No. 5,681,941.

In certain embodiments, at least one nucleobase of a modified oligonucleotide is a modified nucleobase selected from modified adenine (A) having a structure represented by structure I, modified guanine (G) having a structure represented by structure II, modified thymine (T) or modified uracil (U) having a structure represented by structure III, and modified cytosine (C) having a structure represented by structure IV.

In certain embodiments, each nucleobase of a modified oligonucleotide is selected from unmodified A, unmodified G, unmodified C, unmodified T, unmodified U, and 5-methylcytosine (^{m}C). 5-methylcytosine is a modified nucleobase having structure IV, where X is O, R^{4C} is NH₂, and R^{5C} is CH₃.

In certain embodiments, each nucleobase of a modified oligonucleotide is selected from unmodified A, unmodified G, unmodified C, unmodified T, unmodified U, ^{m}C and hypoxanthine. Hypoxanthine is a modified nucleobase having structure Ia and is also a modified A represented by structure I, where Y^{7A} is N and R^{7A} is absent; Y^{8A} is C, R^{8A} is H, R^{1A} is H, R^{2A} is H, and R⁶ is oxo.

In certain embodiments, there are no modified nucleobases in a modified oligonucleotide and each nucleobase of a modified oligonucleotide is selected from unmodified A, unmodified G, unmodified C, unmodified T, and unmodified U.

### 3. ModifiedInternucleoside Linkages

In certain embodiments, oligomeric compounds provided herein comprise or consist of a modified oligonucleotide comprising at least one modified internucleoside linkage. The naturally occurring internucleoside linkage of RNA and DNA is a 3′ to 5′ phosphodiester linkage. In certain embodiments, nucleosides of modified oligonucleotides are linked together using one or more modified internucleoside linkages. The two main classes of internucleoside linkages are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linkages include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide.

In certain embodiments, a modified internucleoside linkage is any of those described in WO/2021/030778, incorporated by reference herein. In certain embodiments, a modified internucleoside linkage comprises the formula:

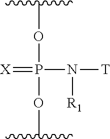

wherein independently for each internucleoside linkage of the modified oligonucleotide:
X is selected from O or S;
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl, substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group; and
R₅ is selected from OCH₃, OH, C₁-C₆ alkyl, and substituted C₁-C₆ alkyl.

In certain embodiments, a modified internucleoside linkage comprises a mesyl phosphoramidate linkage having a formula:

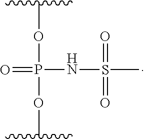 [SMILES: *OP(=O)(NS(C)(=O)=O)O*]

Certain internucleoside linkages having reduced charge (referred to as “neutral internucleoside linkages”) have been described. Such neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl (MOP) (see, e.g., U.S. Pat. No. 9,926,556), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, modified oligonucleotides comprise one or more inverted nucleoside, as shown below:

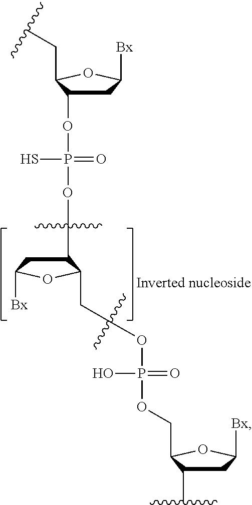

wherein each Bx independently represents any nucleobase.

In certain embodiments, an inverted nucleoside is terminal (i.e., the last nucleoside on one end of an oligonucleotide) and so only one internucleoside linkage depicted above will be present. In certain embodiments, additional features (e.g., a conjugate group) are attached to the inverted nucleoside. Such terminal inverted nucleosides can be attached to either or both ends of an oligonucleotide.

In certain embodiments, inverted nucleosides lack a nucleobase and are referred to herein as inverted sugar moieties. In certain embodiments, an inverted sugar moiety is terminal (i.e., attached to the last nucleoside on one end of an oligonucleotide) and so only one internucleoside linkage above will be present. In certain such embodiments, additional features (e.g., a conjugate group) are attached to the inverted sugar moiety. A terminal inverted sugar moiety can be attached to either or both ends of an oligonucleotide.

In certain embodiments, nucleosides are linked 2′ to 5′ rather than the standard 3′ to 5′ linkage. Such a linkage is illustrated below.

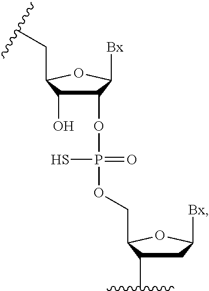

wherein each Bx represents any nucleobase.

In certain embodiments, internucleoside linkages have at least one chiral center. In such embodiments, a chiral atom can be prepared as a racemic mixture, or as separate enantiomers. Representative internucleoside linkages having a chiral center include but are not limited to alkylphosphonates, mesyl phosphoramidates, and phosphorothioates.

The mesyl phosphoramidate internucleoside linkage comprises a chiral center. In certain embodiments, modified oligonucleotides comprising (Rp) and/or (Sp) mesyl phosphoramidates comprise one or more of the following formulas, respectively, wherein “Bx” indicates a nucleobase:

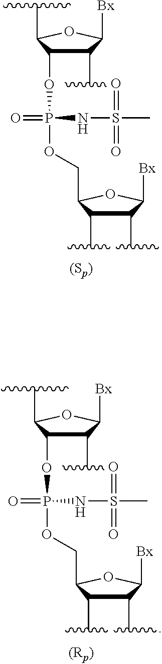

The phosphorothioate internucleoside linkage comprises a chiral center. In certain embodiments, modified oligonucleotides comprising (Rp) and/or (Sp) phosporothioates comprise one or more of the following formulas, respectively, wherein “Bx” indicates a nucleobase:

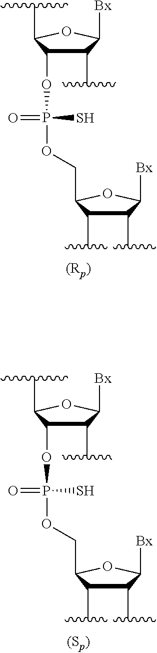

Modified oligonucleotides comprising internucleoside linkages having a chiral center can be prepared as populations of modified oligonucleotides comprising stereorandom internucleoside linkages, or as populations of modified oligonucleotides comprising internucleoside linkages containing chiral centers in particular stereochemical configurations. In certain embodiments, populations of modified oligonucleotides comprise one or more phosphorothioate internucleoside linkages wherein all of the phosphorothioate internucleoside linkages are stereorandom. In certain embodiments, populations of modified oligonucleotides comprise one or more mesyl phosphoramidate internucleoside linkages wherein all of the mesyl phosphoramidate internucleoside linkages are stereorandom. Such modified oligonucleotides can be generated using synthetic methods that result in random selection of the stereochemical configuration of each phosphorothioate and/or mesyl phosphoramidate linkage. Nonetheless, each individual phosphorothioate and/or mesyl phosphoramidate of each individual oligonucleotide molecule has a defined stereoconfiguration. In certain embodiments, populations of modified oligonucleotides are enriched for modified oligonucleotides comprising one or more particular phosphorothioate and/or mesyl phosphoramidate internucleoside linkages in a particular, independently selected stereochemical configuration (e.g., Rp or Sp). In certain embodiments, the particular configuration of the particular phosphorothioate and/or mesyl phosphoramidate linkage is present in at least 65% of the molecules in the population. In certain embodiments, the particular configuration of the particular phosphorothioate and/or mesyl phosphoramidate linkage is present in at least 70% of the molecules in the population. In certain embodiments, the particular configuration of the particular phosphorothioate and/or mesyl phosphoramidate linkage is present in at least 80% of the molecules in the population. In certain embodiments, the particular configuration of the particular phosphorothioate and/or mesyl phosphoramidate linkage is present in at least 90% of the molecules in the population. In certain embodiments, the particular configuration of the particular phosphorothioate and/or mesyl phosphoramidate linkage is present in at least 99% of the molecules in the population. Such chirally enriched populations of modified oligonucleotides can be generated using synthetic methods known in the art, e.g., methods described in Oka et al., J. Am. Chem. Soc. 125, 8307-8317 (2003), Wan et al. Nuc. Acid. Res. 42, 13456 (2014), and WO 2017/015555.

As used herein, “chirally enriched” in reference to a population means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom as defined herein. Chirally enriched populations of molecules having multiple chiral centers within each molecule may contain one or more stereorandom chiral centers. In certain embodiments, the molecules are modified oligonucleotides. In certain embodiments, the molecules are oligomeric compounds comprising modified oligonucleotides. In certain embodiments, the chiral center is at the phosphorous atom of a phosphorothioate internucleoside linkage. In certain embodiments, the chiral center is at the phosphorous atom of a mesyl phosphoramidate internucleoside linkage. In certain embodiments, a population of modified oligonucleotides is enriched for modified oligonucleotides having at least one indicated phosphorothioate and/or mesyl phosphoramidate in the (Sp) configuration. In certain embodiments, a population of modified oligonucleotides is enriched for modified oligonucleotides having at least one phosphorothioate and/or mesyl phosphoramidate in the (Rp) configuration. Unless otherwise indicated, chiral internucleoside linkages of modified oligonucleotides described herein can be stereorandom or in a particular stereochemical configuration. In certain embodiments, modified oligonucleotides of a chirally enriched population are enriched for D-D ribosyl sugar moieties, and all of the phosphorothioate internucleoside linkages are stereorandom and all of the mesyl phosphoramidate internucleoside linkages are stereorandom. In certain embodiments, modified oligonucleotides of a chirally enriched population are enriched for D-D ribosyl sugar moieties, at least one particular phosphorothioate internucleoside linkage in a particular stereochemical configuration is enriched, and all of the mesyl phosphoramidate internucleoside linkages are stereorandom. In certain embodiments, modified oligonucleotides of a chirally enriched population are enriched for D-D ribosyl sugar moieties, at least one particular mesyl phosphoramidate internucleoside linkage in a particular stereochemical configuration is enriched, and all of the phosphorothioate internucleoside linkages are stereorandom. In certain embodiments, modified oligonucleotides of a chirally enriched population are enriched for both β-D ribosyl sugar moieties and at least one, particular phosphorothioate internucleoside linkage in a particular stereochemical configuration and at least one particular mesyl phosphoramidate internucleoside linkage in a particular stereochemical configuration is enriched.

### B. Motifs

In certain embodiments, modified oligonucleotides comprise one or more modified nucleosides comprising a modified sugar moiety or sugar surrogate. In certain embodiments, modified oligonucleotides comprise one or more modified nucleosides comprising a modified nucleobase. In certain embodiments, modified oligonucleotides comprise one or more modified internucleoside linkage. In certain such embodiments, the modified, unmodified, and differently modified sugar moieties, sugar surrogates, nucleobases, and/or internucleoside linkages of a modified oligonucleotide define a pattern or motif. In certain embodiments, the patterns of sugar moieties, sugar surrogates, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the nucleobase sequence).

### 1. Sugar Motifs

In certain embodiments, oligonucleotides comprise one or more type of modified sugar, sugar surrogate, and/or unmodified sugar moiety arranged along the oligonucleotide or portion thereof in a defined pattern or sugar motif. In certain instances, such sugar motifs include but are not limited to any of the sugar modifications and sugar surrogates discussed herein. In certain embodiments, the sugar moiety of at least one nucleoside of an antisense oligonucleotide is a modified sugar moiety or sugar surrogate. In certain embodiments, the sugar moiety of at least one nucleoside of a sense oligonucleotide is a modified sugar moiety or sugar surrogate.

In certain embodiments, modified oligonucleotides comprise or consist of a region having a fully modified sugar motif. In such embodiments, each nucleoside of the fully modified region of the modified oligonucleotide comprises a modified sugar moiety or sugar surrogate. In certain embodiments, each nucleoside of the entire modified oligonucleotide comprises a modified sugar moiety or sugar surrogate and the oligonucleotide is referred to as a fully modified oligonucleotide. In certain embodiments, modified oligonucleotides comprise or consist of a region having a fully modified sugar motif, wherein each nucleoside within the fully modified region comprises the same modified sugar moiety or sugar surrogate, referred to herein as a uniformly modified sugar motif. In certain embodiments, a fully modified oligonucleotide is a uniformly modified oligonucleotide. In certain embodiments, each nucleoside of a uniformly modified oligonucleotide comprises the same 2′-modification. In certain embodiments, every other nucleoside of a uniformly modified oligonucleotide comprises the same 2′-modification, resulting in an alternating 2′-modifications. In certain embodiments, neighboring nucleosides comprise different 2′-modification, and every other nucleoside of a uniformly modified oligonucleotide comprises the same 2′-modification, resulting in a uniform, alternating 2′-modification motif.

In certain embodiments, at least one nucleoside of a modified oligonucleotide comprises a 2′-OMe sugar moiety (i.e., a 2′-OMe modified nucleoside). In certain embodiments, at least 2 nucleosides comprise 2′-OMe sugar moieties. In certain embodiments, at least 5 nucleosides comprise 2′-OMe sugar moieties. In certain embodiments, at least 8 nucleosides comprise 2′-OMe sugar moieties. In certain embodiments, at least 10 nucleosides comprise 2′-OMe sugar moieties. In certain embodiments, at least 12 nucleosides comprise 2′-OMe sugar moieties. In certain embodiments, at least 13 nucleosides comprise 2′-OMe sugar moieties. In certain embodiments, at least 14 nucleosides comprise 2′-OMe sugar moieties. In certain embodiments, at least 15 nucleosides comprise 2′-OMe sugar moieties. In certain embodiments, at least 16 nucleosides comprise 2′-OMe sugar moieties. In certain embodiments, at least 17 nucleosides comprise 2′-OMe sugar moieties. In certain such embodiments, at least 18 nucleosides comprise 2′-OMe sugar moieties. In certain such embodiments, at least 20 nucleosides comprise 2′-OMe sugar moieties. In certain such embodiments, at least 21 nucleosides comprise 2′-OMe sugar moieties.

In certain embodiments, at least one nucleoside of a modified oligonucleotide comprises a 2′-F sugar moiety (i.e., a 2′-F modified nucleoside). In certain embodiments, at least 2 nucleosides comprise 2′-F sugar moieties. In certain embodiments, at least 3 nucleosides comprise 2′-F sugar moieties. In certain embodiments, at least 4 nucleosides comprise 2′-F sugar moieties. In certain embodiments, at least 6 nucleosides comprise 2′-F sugar moieties. In certain embodiments, at least 8 nucleosides comprise 2′-F sugar moieties. In certain embodiments, at least 10 nucleosides comprise 2′-F sugar moieties. In certain embodiments, at least 11 nucleosides comprise 2′-F sugar moieties. In certain embodiments, one, but not more than one nucleoside comprises a 2′-F sugar moiety. In certain embodiments, 1 or 2 nucleosides comprise 2′-F sugar moieties. In certain embodiments, 1-3 nucleosides comprise 2′-F sugar moieties. In certain embodiments, at least 1-4 nucleosides comprise 2′-F sugar moieties. In certain embodiments, antisense oligonucleotides have a block of 2-4 contiguous 2′-F modified nucleosides. In certain embodiments, 4 nucleosides of an antisense oligonucleotide are 2′-F modified nucleosides and 2 or 3 of those 2′-F modified nucleosides are contiguous. In certain embodiments, 1, 2, 3, or 4 nucleosides of an antisense oligonucleotide are 2′-F modified nucleosides and each of those 2′-F modified nucleosides are non-contiguous. In certain such embodiments the remainder of the nucleosides are 2′-OMe modified nucleosides.

In certain embodiments, at least one nucleoside of a modified oligonucleotide comprises a 2′-deoxy sugar moiety that has no additional modifications. In certain embodiments, at least one nucleoside comprises a 2′-deoxy sugar moiety. In certain embodiments, at least 2 nucleosides comprise a 2′-deoxy sugar moiety. In certain embodiments, at least 3 nucleosides comprise a 2′-deoxy sugar moiety. In certain embodiments, at least 4 nucleosides comprise a 2′-deoxy sugar moiety. In certain embodiments, one, but not more than one nucleoside comprises a 2′-deoxy sugar moiety. In certain embodiments, 1 or 2 nucleosides comprise a 2′-deoxy sugar moiety. In certain embodiments, 1-3 nucleosides comprise a 2′-deoxy sugar moiety. In certain embodiments, at least 1-4 nucleosides comprise a 2′-deoxy sugar moiety. In certain embodiments, 1, 2, 3, or 4 nucleosides of an antisense oligonucleotide is/are a 2′-deoxynucleoside and each 2′-deoxynucleoside is not immediately adjacent to another 2′-deoxynucleoside. In certain embodiments, 1, or 2 nucleosides of an antisense oligonucleotide are a 2′-deoxy nucleoside and each 2′-deoxynucleoside is not immediately adjacent to another 2′-deoxynucleoside. In certain embodiments, 1, or 2 nucleosides of a sense oligonucleotide are a 2′-deoxynucleoside and each 2′-deoxy nucleoside is not immediately adjacent to another 2′-deoxynucleoside. In certain embodiments, 2 nucleosides of an antisense oligonucleotide are 2′-deoxynucleosides and one nucleoside of a sense oligonucleotide is a 2′-deoxynucleoside. In certain embodiments, 2 nucleosides of an antisense oligonucleotide are 2′-deoxynucleosides and no nucleoside of a sense oligonucleotide is a 2′-deoxynucleoside. In certain embodiments, 2 nucleosides of an antisense oligonucleotide are 2′-deoxynucleosides.

In certain embodiments, at least one nucleoside of an antisense oligonucleotide and/or a sense oligonucleotide comprises a modified sugar moiety and/or sugar surrogate. In certain embodiments, a sugar moiety of an antisense oligonucleotide is modified, wherein the modified sugar modifications and/or sugar surrogate is selected from 2′-F, 2′-MOE, 2′-OMe, 2′-deoxy, and FHNA. In certain embodiments, a sugar motif (from 5′ to 3′) of the antisense oligonucleotide is selected from yfyyyfyyyyyyyfyfyyyyyyy, efyyyfyyyyyyyfyfyyyyyee, efyyyyyyyyyyyfyyyyyyyee, efyyydyyyyyyyfyfyyyyyee, efyyydyyyyyyydydyyyyyyy, efyyydyyyyyyyfyfyyyyyyy, efyydydyyyyyyfyfyyyyyyy, efyyyfyyyyyyyfyyyyyyyee, efyyyyyyyyyyyfyfyyyyyee, efyyyfyyyyyyyfyyyyyyyyy, efyyyyyyyyyyyfyfyyyyyyy, efyyyfyyyyyyyfyfyyyyyey, efyyyfyyyyyyyfydyyyyyee, efyyyfyyyyyyyfydyyyyyyy, efyydydyyyyyyfyfyyyyyee, efyyyfyyyyyyyfyfyyyee, e[FHNA]yydydyyyyfyfyfyyyyyyy, e[FHNA]yyyfyyyyyyyfyfyyyyyyy, e[FHNA]yyyfyyyyyyyfyfyyyyyee, and e[FHNA]yyyyyyyyyyy[FHNA]yyyyyyyee, wherein each ‘y’ represents a 2′-OMe sugar moiety, each ‘f’ represents a 2′-F sugar moiety, each ‘[FHNA]’ represents a 3′-fluoro-hexitol sugar moiety, each ‘d’ represents a 2′-deoxy sugar moiety, and each ‘e’ represents a 2′-MOE sugar moiety. In certain embodiments, a sugar moiety of a sense oligonucleotide is modified, wherein the modified sugar moiety is selected from 2′-F, 2′-MOE, 2′-OMe, and 2′-deoxy. In certain embodiments, a sugar motif (from 5′ to 3′) of a sense oligonucleotide is selected from among: yyyyyyfyfffyyyyyyyyyy, yyyyyyyyyyyyyyyyyyyyy, yyyyyyyyyffyyyyyyyyyy, yyyyyyfyfyfyyyyyyyyyy, yyyyyyyyyydyyyyyyyyyy, eeyyyyfyfffyyyyyyyyee, eeyyyyyyyffyyyyyyyyee, and eeyyyyfyfyfyyyyyyyyee, wherein each ‘y’ represents a 2′-OMe sugar moiety, each ‘f’ represents a 2′-F sugar moiety, each ‘d’ represents a 2′-deoxy sugar moiety, and each ‘e’ represents a 2′-MOE sugar moiety.

### 2. Nucleobase Motifs

In certain embodiments, oligonucleotides comprise modified and/or unmodified nucleobases arranged along the oligonucleotide or portion thereof in a defined pattern or motif. In certain embodiments, at least one nucleobase is modified. In certain embodiments, none of the nucleobases are modified. In certain embodiments, at least one purine and/or at least pyrimidine is modified. In certain embodiments, at least one adenine is modified. In certain embodiments, at least one guanine is modified. In certain embodiments, at least one thymine is modified. In certain embodiments, at least one uracil is modified. In certain embodiments, at least one cytosine is modified. In certain embodiments, at least one of the cytosine nucleobases in a modified oligonucleotide is 5-methylcytosine. In certain embodiments, all of the cytosine nucleobases are 5-methylcytosines and all of the other nucleobases of the modified oligonucleotide are unmodified nucleobases. In certain embodiments, one or two of the cytosine nucleobases are 5-methylcytosine and all of the other nucleobases of the modified oligonucleotide are unmodified nucleobases. In certain embodiments, each nucleobase is selected from 5-methylcytosine, unmodified cytosine, unmodified thymine, unmodified uracil, unmodified adenine, and unmodified guanine, hypoxanthine, and xanthine. In certain embodiments, each nucleobase is selected from 5-methylcytosine, unmodified cytosine, unmodified thymine, unmodified adenine, and unmodified guanine. In certain embodiments, each nucleobase is selected from unmodified cytosine, unmodified thymine, unmodified uracil, unmodified adenine, and unmodified guanine and hypoxanthine. In certain embodiments, each nucleobase is selected from unmodified cytosine, unmodified thymine, unmodified adenine, and unmodified guanine and hypoxanthine. In certain embodiments, each nucleobase is selected from unmodified cytosine, unmodified thymine, unmodified uracil, unmodified adenine, and unmodified guanine. In certain embodiments, each nucleobase is selected from unmodified cytosine, unmodified thymine, unmodified adenine, and unmodified guanine.

### 3. Internucleoside Linkage Motifs

In certain embodiments, oligonucleotides comprise modified and unmodified internucleoside linkages arranged along the oligonucleotide or portion thereof in a defined pattern or motif. In certain embodiments, each internucleoside linkage is a phosphodiester internucleoside linkage (P═O). In certain embodiments, each internucleoside linkage of a modified oligonucleotide is a phosphorothioate internucleoside linkage (P═S). In certain embodiments, each internucleoside linkage of a modified oligonucleotide is independently selected from a phosphorothioate internucleoside linkage, a mesyl phosphoramidate internucleoside linkage, and phosphodiester internucleoside linkage. In certain embodiments, each internucleoside linkage of a modified oligonucleotide is independently selected from a phosphorothioate internucleoside linkage and a phosphodiester internucleoside linkage. In certain embodiments, each phosphorothioate internucleoside linkage is independently selected from a stereorandom phosphorothioate, a (Sp) phosphorothioate, and a (Rp) phosphorothioate. In certain embodiments, each mesyl phosphoramidate internucleoside linkage is independently selected from a stereorandom mesyl phosphoramidate, a (Sp) mesyl phosphoramidate, and a (Rp) mesyl phosphoramidate.

In certain embodiments, at least one internucleoside linkage of the antisense oligonucleotide is a modified internucleoside linkage. In certain embodiments, the 5′-most internucleoside linkage (i.e., linking the first nucleoside from the 5′-end to the second nucleoside from the 5′-end) is modified. In certain embodiments, the two 5′-most internucleoside linkages are modified. In certain embodiments, the first one or 2 internucleoside linkages from the 3′-end are modified. In certain embodiments, the modified internucleoside linkage is a phosphorothioate internucleoside linkage. In certain embodiments, the modified internucleoside linkage is a mesyl phosphoramidate internucleoside linkage. In certain embodiments, the remaining internucleoside linkages are all unmodified phosphodiester internucleoside linkages. In certain embodiments an antisense oligonucleotide has an internucleoside linkage motif (from 5′ to 3′) of: ssooooooooooooooooooss, wherein each ‘o’ represents a phosphodiester internucleoside linkage and each ‘s’ represents a phosphorothioate internucleoside linkage. In certain embodiments an antisense oligonucleotide has an internucleoside linkage motif (from 5′ to 3′) of: ssooosooooooooooooooss, wherein each ‘o’ represents a phosphodiester internucleoside linkage and each ‘s’ represents a phosphorothioate internucleoside linkage. In certain embodiments an antisense oligonucleotide has an internucleoside linkage motif (from 5′ to 3′) of: ssoososoooooooooooooss, wherein each ‘o’ represents a phosphodiester internucleoside linkage and each ‘s’ represents a phosphorothioate internucleoside linkage. In certain embodiments an antisense oligonucleotide has an internucleoside linkage motif (from 5′ to 3′) of: ssooooooooooooooooss, wherein each ‘o’ represents a phosphodiester internucleoside linkage and each ‘s’ represents a phosphorothioate internucleoside linkage. In certain embodiments an antisense oligonucleotide has an internucleoside linkage motif (from 5′ to 3′) of: ss(o)ₙ₋₅ss, wherein each ‘o’ represents a phosphodiester internucleoside linkage and each ‘s’ represents a phosphorothioate internucleoside linkage, and wherein n represents the number of nucleobases in the oligonucleotide.

In certain embodiments, at least one internucleoside linkage of the sense oligonucleotide is a modified internucleoside linkage. In certain embodiments, the 5′-most internucleoside linkage (i.e., linking the first nucleoside from the 5′-end to the second nucleoside from the 5′-end) is modified. In certain embodiments, the two 5′-most internucleoside linkages are modified. In certain embodiments, the first one or 2 internucleoside linkages from the 3′-end are modified. In certain embodiments, the modified internucleoside linkage is a phosphorothioate internucleoside linkage. In certain embodiments, the modified internucleoside linkage is a mesyl phosphoramidate internucleoside linkage. In certain embodiments, the remaining internucleoside linkages are all unmodified phosphodiester linkages. In certain embodiments a sense oligonucleotide has an internucleoside linkage motif (from 5′ to 3′) of: ssooooooooooooooooss, wherein each ‘o’ represents a phosphodiester internucleoside linkage and each ‘s’ represents a phosphorothioate internucleoside linkage. In certain embodiments a sense oligonucleotide has an internucleoside linkage motif (from 5′ to 3′) of: ssoooooozozoooooooss, wherein each ‘o’ represents a phosphodiester internucleoside linkage and each ‘s’ represents a phosphorothioate internucleoside linkage and each ‘z’ represents a mesyl phosphoramidate internucleoside linkage. In certain embodiments a sense oligonucleotide has an internucleoside linkage motif (from 5′ to 3′) of: ssooooooosooooooooss, wherein each ‘o’ represents a phosphodiester internucleoside linkage and each ‘s’ represents a phosphorothioate internucleoside linkage. In certain embodiments a sense oligonucleotide has an internucleoside linkage motif (from 5′ to 3′) of: ssooooooooooooooss, wherein each ‘o’ represents a phosphodiester internucleoside linkage and each ‘s’ represents a phosphorothioate internucleoside linkage. In certain embodiments a sense oligonucleotide has an internucleoside linkage motif (from 5′ to 3′) of: ss(o)ₙ₋₅ss, wherein each ‘o’ represents a phosphodiester internucleoside linkage and each ‘s’ represents a phosphorothioate internucleoside linkage, and wherein n represents the number of nucleobases in the oligonucleotide.

### C. Lengths

It is possible to increase or decrease the length of an oligonucleotide without eliminating activity. For example, in Woolf et al. (Proc. Natl. Acad. Sci. USA 89:7305-7309, 1992), a series of oligonucleotides 13-25 nucleobases in length were tested for their ability to induce cleavage of a target RNA in an oocyte injection model. Oligonucleotides 25 nucleobases in length with 8 or 11 mismatch bases near the ends of the oligonucleotides were able to direct specific cleavage of the target RNA, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (e.g., modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 27, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments, modified oligonucleotides (including, e.g., antisense oligonucleotides, sense oligonucleotides) comprise 16 linked nucleosides having no more than 1 to 3 mismatches to a target sequence. In certain embodiments, modified oligonucleotides (including, e.g., antisense oligonucleotides, sense oligonucleotides) comprise 17 linked nucleosides having no more than 1 to 3 mismatches to a target sequence. In certain embodiments, modified oligonucleotides (including, e.g., antisense oligonucleotides, sense oligonucleotides) comprise 18 linked nucleosides having no more than 1 to 3 mismatches to a target sequence. In certain embodiments, modified oligonucleotides (including, e.g., antisense oligonucleotides, sense oligonucleotides) comprise 19 linked nucleosides having no more than 1 to 3 mismatches to a target sequence. In certain embodiments, modified oligonucleotides (including, e.g., antisense oligonucleotides, sense oligonucleotides) comprise 20 linked nucleosides having no more than 1 to 3 mismatches to a target sequence. In certain embodiments, modified oligonucleotides (including, e.g., antisense oligonucleotides, sense oligonucleotides) comprise 21 linked nucleosides having no more than 1 to 3 mismatches to a target sequence. In certain embodiments, modified oligonucleotides (including, e.g., antisense oligonucleotides, sense oligonucleotides) comprise 22 linked nucleosides having no more than 1 to 3 mismatches to a target sequence. In certain embodiments, modified oligonucleotides (including, e.g., antisense oligonucleotides, sense oligonucleotides) comprise 23 linked nucleosides having no more than 1 to 3 mismatches to a target sequence.

In certain embodiments, modified oligonucleotides (including, e.g., antisense oligonucleotides, sense oligonucleotides) consist of 16 linked nucleosides. In certain embodiments, modified oligonucleotides (including antisense oligonucleotides) consist of 17 linked nucleosides. In certain embodiments, modified oligonucleotides (including, e.g., antisense oligonucleotides, sense oligonucleotides) consist of 18 linked nucleosides. In certain embodiments, modified oligonucleotides (including, e.g., antisense oligonucleotides, sense oligonucleotides) consist of 19 linked nucleosides. In certain embodiments, modified oligonucleotides (including, e.g., antisense oligonucleotides, sense oligonucleotides) consist of 20 linked nucleosides. In certain embodiments, modified oligonucleotides (including, e.g., antisense oligonucleotides, sense oligonucleotides) consist of 21 linked nucleosides. In certain embodiments, modified oligonucleotides (including, e.g., antisense oligonucleotides, sense oligonucleotides) consist of 22 linked nucleosides. In certain embodiments, modified oligonucleotides (including, e.g., antisense oligonucleotides, sense oligonucleotides) consist of 23 linked nucleosides.

In certain embodiments, antisense oligonucleotides consist of 12-30 linked nucleosides. In certain embodiments, antisense oligonucleotides consist of 17-25 linked nucleosides. In certain embodiments, antisense oligonucleotides consist of 17-23 linked nucleosides. In certain embodiments, antisense oligonucleotides consist of 17-21 linked nucleosides. In certain embodiments, antisense oligonucleotides consist of 18-30 linked nucleosides. In certain embodiments, antisense oligonucleotides consist of 20-30 linked nucleosides. In certain embodiments, antisense oligonucleotides consist of 21-30 linked nucleosides. In certain embodiments, antisense oligonucleotides consist of 23-30 linked nucleosides. In certain embodiments, antisense oligonucleotides consist of 18-25 linked nucleosides. In certain embodiments, antisense oligonucleotides consist of 20-22 linked nucleosides. In certain embodiments, antisense oligonucleotides consist of 21-23 linked nucleosides. In certain embodiments, antisense oligonucleotides consist of 23-24 linked nucleosides. In certain embodiments, antisense oligonucleotides consist of 20 linked nucleosides. In certain embodiments, antisense oligonucleotides consist of 21 linked nucleosides. In certain embodiments, antisense oligonucleotides consist of 22 linked nucleosides. In certain embodiments, antisense oligonucleotides consist of 23 linked nucleosides. In certain embodiments, antisense oligonucleotides consist of 20 linked nucleosides having no more than 1 to 3 mismatches to a target sequence. In certain embodiments, antisense oligonucleotides consist of 21 linked nucleosides having no more than 1 to 3 mismatches to a target sequence. In certain embodiments, antisense oligonucleotides consist of 22 linked nucleosides having no more than 1 to 3 mismatches to a target sequence. In certain embodiments, antisense oligonucleotides consist of 23 linked nucleosides having no more than 1 to 3 mismatches to a target sequence.

In certain embodiments, sense oligonucleotides consist of 12-30 linked nucleosides. In certain embodiments, sense oligonucleotides consist of 16-25 linked nucleosides. In certain embodiments, sense oligonucleotides consist of 16-23 linked nucleosides. In certain embodiments, sense oligonucleotides consist of 16-21 linked nucleosides. In certain embodiments, sense oligonucleotides consist of 16-30 linked nucleosides. In certain embodiments, sense oligonucleotides consist of 18-30 linked nucleosides. In certain embodiments, sense oligonucleotides consist of 19-30 linked nucleosides. In certain embodiments, sense oligonucleotides consist of 19-25 linked nucleosides. In certain embodiments, sense oligonucleotides consist of 18-25 linked nucleosides. In certain embodiments, sense oligonucleotides consist of 18-20 linked nucleosides. In certain embodiments, sense oligonucleotides consist of 19-21 linked nucleosides. In certain embodiments, sense oligonucleotides consist of 18 linked nucleosides. In certain embodiments, sense oligonucleotides consist of 19 linked nucleosides. In certain embodiments, sense oligonucleotides consist of 20 linked nucleosides. In certain embodiments, sense oligonucleotides consist of 21 linked nucleosides. In certain embodiments, sense oligonucleotides consist of 18 linked nucleosides having no more than 1 to 3 mismatches to a target sequence. In certain embodiments, sense oligonucleotides consist of 19 linked nucleosides having no more than 1 to 3 mismatches to a target sequence. In certain embodiments, sense oligonucleotides consist of 20 linked nucleosides having no more than 1 to 3 mismatches to a target sequence. In certain embodiments, sense oligonucleotides consist of 21 linked nucleosides having no more than 1 to 3 mismatches to a target sequence.

### D. Oligomeric Modifications

Provided oligomeric compounds comprise one or more modifications, e.g., sugar, nucleobase, internucleoside linkage, and/or combinations thereof, incorporated into a modified oligonucleotide. In certain embodiments, a modified oligonucleotide is characterized by modification motif(s) and overall length. In certain embodiments, such parameters are each independent of one another. Thus, unless otherwise indicated, each internucleoside linkage of an oligonucleotide having one or more modified sugar moiety or sugar surrogate and/or sugar motif, independently, is modified or unmodified and may or may not follow the modification pattern of the sugar modifications. For example, internucleoside linkages within regions of an oligonucleotide comprising certain sugar modifications may be the same or different from one another and may be the same or different from the internucleoside linkages of the region of the oligonucleotide comprising different sugar modifications. Likewise, such modified oligonucleotides may comprise one or more modified nucleobase independent of the pattern of the sugar modifications and independent of the internucleoside linkages. Unless specifically indicated, all modifications are independent of nucleobase sequence. Furthermore, each modification, whether internucleoside linkage, modified sugar moiety, sugar surrogate, modified nucleobase, of an antisense oligonucleotide is independent of each modification of a sense oligonucleotide binding partner unless specifically indicated otherwise.

### E. Nucleobase Sequence

In certain embodiments, oligonucleotides (unmodified or modified oligonucleotides) are further described by their nucleobase sequence. In certain embodiments oligonucleotides have a nucleobase sequence that is complementary to a nucleobase sequence of a second oligonucleotide or an identified reference nucleic acid, such as a target nucleic acid or portion thereof. In certain such embodiments, a region of an oligonucleotide has a nucleobase sequence that is complementary to a nucleobase sequence of a second oligonucleotide or an identified reference nucleic acid, such as a target nucleic acid or portion thereof. In certain embodiments, the nucleobase sequence of a region or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid or portion thereof.

### II. Oligomeric Duplexes

In certain embodiments, an oligomeric compound provided herein comprises a first modified oligonucleotide having a nucleobase sequence complementary to a sequence in a PLN target nucleic acid paired with a second modified oligonucleotide to form an oligomeric duplex. Such oligomeric duplex comprises a first oligomeric compound comprising a modified oligonucleotide having a portion complementary to a sequence in a PLN target nucleic acid and a second oligomeric compound comprising a modified oligonucleotide having a portion complementary to the first modified oligonucleotide. In certain embodiments, the first oligomeric compound of an oligomeric duplex comprises or consists of a first modified oligonucleotide and optionally a conjugate group and/or terminal group; and the second oligomeric compound of the oligomeric duplex comprises or consists of a second modified oligonucleotide and optionally a terminal group and/or a conjugate group. Either or both oligomeric compounds of an oligomeric duplex may comprise a conjugate group. In certain embodiments the sense oligomeric compound of an oligomeric duplex comprises a conjugate group attached at the 5′ or 3′ end of the sense oligomeric compound. Either or both oligomeric compounds of an oligomeric duplex may comprise a terminal group. In certain embodiments the antisense oligomeric compound of an oligomeric duplex comprises a 5′-terminal group. One or both oligonucleotides of each oligomeric compound of an oligomeric duplex may include one or more (e.g., one, two, three, or more) terminal nucleosides that form an overhang at one (i.e., the 5′ end or 3′ end) or both ends of the oligomeric duplex. In certain embodiments an overhang is one or two nucleosides (e.g., of an antisense oligomeric compound, sense oligomeric compound, antisense oligonucleotide, sense oligonucleotide) of an oligomeric duplex. In certain embodiments the terminal one or two nucleosides at the 3′ end or at the 5′ end of an antisense or sense oligomeric compound or an antisense or sense oligonucleotide are overhang nucleosides of an oligomeric duplex. In certain embodiments the overhang nucleosides are adenosine, inosine, or thymidine. In certain embodiments one or both ends of the oligomeric duplex are blunt ends. In certain embodiments, the two oligonucleotides have at least one mismatch relative to one another. In certain embodiments, the oligomeric duplex is an antisense compound.

In certain embodiments, an overhang is one or two nucleosides of a first oligomeric compound or first modified oligonucleotide of an oligomeric duplex. In certain embodiments the last two 3′-nucleosides (i.e., the 3′-terminal nucleoside and the nucleoside immediately 5′ of the 3′-terminal nucleoside) of a first oligomeric compound or first modified oligonucleotide of an oligomeric duplex are overhang nucleosides. In certain embodiments, a 3′ overhang nucleoside of a first oligomeric compound or first modified oligonucleotide of an oligomeric duplex comprises an unmodified adenine, unmodified thymine, unmodified uracil or hypoxanthine nucleobase. In certain embodiments, a 3′ overhang of a first oligomeric compound or first modified oligonucleotide of an oligomeric duplex comprises two nucleosides, each comprising a nucleobase independently selected from an unmodified adenine, an unmodified thymine, an unmodified uracil and a hypoxanthine. In certain embodiments, the last two nucleosides at the 3′ end of a first oligomeric compound or first modified oligonucleotide of an oligomeric duplex having a 3′ overhang each comprise a nucleobase independently selected from an unmodified adenine, unmodified thymine, unmodified uracil and hypoxanthine. In certain embodiments, a 3′-overhang nucleoside of a first oligomeric compound or first modified oligonucleotide of an oligomeric duplex comprises an unmodified adenine nucleobase or hypoxanthine nucleobase. In certain embodiments, a 3′ overhang of a first oligomeric compound or first modified oligonucleotide of an oligomeric duplex comprises two nucleosides, each comprising a nucleobase independently selected from an unmodified adenine nucleobase or a hypoxanthine nucleobase. In certain embodiments, the last two terminal nucleosides at the 3′ end of a first oligomeric compound or first modified oligonucleotide of an oligomeric duplex having a 3′ overhang each independently comprise an unmodified adenine nucleobase or a hypoxanthine nucleobase. In certain embodiments, the last two terminal nucleosides at the 3′ end of a first oligomeric compound or first modified oligonucleotide of an oligomeric duplex having a 3′ overhang comprise an unmodified adenine nucleobase. In certain embodiments, the last two terminal nucleosides at the 3′ end of a first oligomeric compound or first modified oligonucleotide of an oligomeric duplex having a 3′ overhang each comprise a hypoxanthine nucleobase. In certain embodiments, the 3′-terminal nucleoside and the nucleoside immediately 5′ of the 3′-terminal nucleoside of a first oligomeric compound or first modified oligonucleotide of an oligomeric duplex having a 3′ overhang comprise an unmodified adenine nucleobase and a hypoxanthine nucleobase, respectively. In certain embodiments, the 3′-terminal nucleoside and the nucleoside immediately 5′ of the 3′-terminal nucleoside of a first oligomeric compound or first modified oligonucleotide of an oligomeric duplex having a 3′ overhang comprise a hypoxanthine nucleobase and an unmodified adenine nucleobase, respectively.

In certain embodiments an overhang of an oligomeric duplex is the 3′-terminal nucleoside of a first oligomeric compound or first modified oligonucleotide of the oligomeric duplex. In certain embodiments the 3′-terminal nucleoside of a first oligomeric compound or first modified oligonucleotide of the oligomeric duplex having a 3′ overhang comprises an unmodified adenine, unmodified thymine, unmodified uracil or hypoxanthine nucleobase. In certain embodiments the 3′-terminal nucleoside of a first oligomeric compound or first modified oligonucleotide of the oligomeric duplex having a 3′ overhang comprises an unmodified adenine nucleobase or hypoxanthine nucleobase. In certain embodiments the 3′-terminal nucleoside of a first oligomeric compound or first modified oligonucleotide of the oligomeric duplex having a 3′ overhang comprises an unmodified adenine nucleobase. In certain embodiments the 3′-terminal nucleoside of a first oligomeric compound or first modified oligonucleotide of the oligomeric duplex having a 3′ overhang comprises a hypoxanthine nucleobase.

In certain embodiments, one or both ends of an oligomeric duplex are blunt ends. In certain embodiments, one end of an oligomeric duplex is blunt. In certain embodiments, both ends of an oligomeric duplex are blunt. In certain embodiments, the 5′-terminal nucleoside of a first oligomeric compound or a first modified oligonucleotide of an oligomeric duplex having at least one blunt end comprises an unmodified thymine nucleobase. In certain embodiments, the 3′-terminal nucleoside of a first oligomeric compound or a first modified oligonucleotide of an oligomeric duplex having at least one blunt end comprises an unmodified guanine or unmodified uracil nucleobase. In certain embodiments, the 5′-terminal nucleoside of a first oligomeric compound or a first modified oligonucleotide of an oligomeric duplex having two blunt ends comprises an unmodified thymine nucleobase and the 3′-terminal nucleoside of the first oligomeric compound or first modified oligonucleotide comprises an unmodified guanine or unmodified uracil nucleobase.

In certain embodiments, an oligomeric duplex comprises a first oligomeric compound/second oligomeric compound pair in which the nucleobase sequence of the first modified oligonucleotide and the nucleobase sequence of the second modified oligonucleotide are a duplex comprising any one of the following pairs of SEQ ID NOS: 1033/1039, 1034/1040, 1035/1041, 1036/1042, 1037/1043, 1038/1044, 1036/1288, 1255/1041, 1265/1288, 1265/1042, 1259/1012, 1262/1013, 1268/1015, 1271/1016, 1264/1288, 1264/1042, 1258/1012, 1261/1013, 1267/1015, 1270/1046, 1260/1012, 1263/1013, 1266/1014, 1269/1015, 1272/1016, 1279/1288, 1273/1012, 1276/1013, 1282/1015, 1285/1016, 1280/1288, 1274/1012, 1277/1013, 1283/1015, and 1286/1016, 1275/1012, 1278/1013, 1281/1014, 1284/1015, and 1287/1016.

In certain embodiments, an oligomeric duplex comprises: a first oligomeric compound comprising a first modified oligonucleotide consisting of 12 to 50 linked nucleosides, wherein the first modified oligonucleotide comprises a region having a nucleobase sequence comprising at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases of the nucleobase sequence of any one of SEQ ID Nos: 3-314, 627-782, 939-976, 1033-1038, 1254-1255, and 1258-1287; and a second oligomeric compound comprising a second modified oligonucleotide consisting of 12 to 50 linked nucleosides that contains a region of at least 12 contiguous nucleosides, wherein the nucleobase sequence of the region of at least 12 contiguous nucleosides is at least 80%, at least 90%, at least 95%, or at least 99% or 100% complementary to the nucleobase sequence of an equal length region of the first modified oligonucleotide. In certain embodiments, the nucleobase sequence of the region of the first modified oligonucleotide having a nucleobase sequence comprising at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases of the nucleobase sequence of any one of SEQ ID Nos: 3-314, 627-782, 939-976, 1033-1038, 1254-1255, and 1258-1287 is at least 80% complementary to the nucleobase sequence of an equal length region of a PLN nucleic acid. In certain embodiments, the nucleobase sequence of the second modified oligonucleotide comprises at least 8, at least 9, at least 10, at least 11, at least 12, at least 13, at least 14, at least 15, at least 16, at least 17, at least 18, at least 19, at least 20, or at least 21 contiguous nucleobases of the nucleobase sequence of any one of SEQ ID Nos: 315-626, 783-938, 977-1016, 1027-1032, 1256-1257, and 1288-1290. In certain embodiments, the first oligomeric compound of the oligomeric duplex is an antisense compound, wherein the first modified oligonucleotide is an antisense oligonucleotide. In certain embodiments, the second oligomeric compound of the oligomeric duplex is a sense compound, wherein the second modified oligonucleotide is a sense oligonucleotide. In certain embodiments, the first modified oligonucleotide is an antisense RNAi oligonucleotide. In certain embodiments, the second modified oligonucleotide is a sense RNAi oligonucleotide.

In certain embodiments, an oligomeric duplex comprises: a first oligomeric compound comprising a first modified oligonucleotide consisting of 15 to 30 linked nucleosides, wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases of the nucleobase sequence of any one of SEQ ID Nos: 3-314, 627-782, 939-976, 1033-1038, 1254-1255, and 1258-1287; and a second oligomeric compound comprising a second modified oligonucleotide consisting of 15 to 29 linked nucleosides, wherein the nucleobase sequence of the second modified oligonucleotide comprises at least 8, at least 9, at least 10, at least 11, at least 12, at least 13, at least 14, at least 15, at least 16, at least 17, at least 18, at least 19, at least 20, or at least 21 contiguous nucleobases of the nucleobase sequence of any one of SEQ ID Nos: 315-626, 783-938, 977-1016, 1027-1032, 1256-1257, and 1288-1290. In certain embodiments, the oligomeric duplex is an antisense compound. In certain embodiments, the first oligomeric compound of the oligomeric duplex is an antisense compound, wherein the first modified oligonucleotide is an antisense oligonucleotide. In certain embodiments, the second oligomeric compound of the oligomeric duplex is a sense compound, wherein the second modified oligonucleotide is a sense oligonucleotide. In certain embodiments, the first modified oligonucleotide is an antisense RNAi oligonucleotide. In certain embodiments, the second modified oligonucleotide is a sense RNAi oligonucleotide. In certain embodiments, the nucleobase sequence of the second modified oligonucleotide is at least 90%, 95% or 100% complementary to the nucleobase sequence of an equal length portion of the first modified oligonucleotide.

In certain embodiments, an oligomeric duplex comprises a first oligomeric compound comprising a first modified oligonucleotide consisting of 18 to 30 linked nucleosides, wherein the nucleobase sequence of the first modified oligonucleotide comprises at least 12, at least 13, at least 14, at least 15, at least 16, at least 17, at least 18, at least 19, at least 20, at least 21, at least 22, or 23 contiguous nucleobases of the nucleobase sequence of any one of SEQ ID NO: 3-314, 627-782, 939-976, 1033-1038, 1254-1255, and 1258-1287; and a second oligomeric compound comprising a second modified oligonucleotide consisting of 15 to 29 linked nucleosides, wherein the nucleobase sequence of the second modified oligonucleotide comprises at least 12, at least 13, at least 14, at least 15, at least 16, at least 17, at least 18, at least 19, at least 20, or at least 21 contiguous nucleobases of the nucleobase sequence of any one of SEQ ID Nos: 315-626, 783-938, 977-1016, 1027-1032, 1256-1257, and 1288-1290. In certain embodiments, the first oligomeric compound is an antisense compound, wherein the first modified oligonucleotide is an antisense oligonucleotide. In certain embodiments, the second oligomeric compound is a sense compound, wherein the second modified oligonucleotide is a sense oligonucleotide. In certain embodiments, the first modified oligonucleotide is an antisense RNAi oligonucleotide. In certain embodiments, the second modified oligonucleotide is a sense RNAi oligonucleotide. In certain embodiments, the nucleobase sequence of the second modified oligonucleotide comprises a complementary region of at least 9, at least 10, at least 11, at least 12, at least 13, at least 14, at least 15, at least 16, at least 17, at least 18, at least 19, at least 20, or 21 contiguous nucleobases that is 100% complementary to the nucleobase sequence of an equal length portion of the first modified oligonucleotide. In certain embodiments, the nucleobase sequence of the second modified oligonucleotide is at least 80%, at least 85%, at least 90%, at least 95%, at least 98%, at least 99%, or 100% complementary to the nucleobase sequence of an equal length portion of the first modified oligonucleotide. In certain embodiments, the oligomeric duplex is an antisense compound. In certain embodiments, the nucleobase sequence of the second modified oligonucleotide comprises a complementary region of at least 9, at least 10, at least 11, at least 12, at least 13, at least 14, at least 15, at least 16, at least 17, at least 18, at least 19, at least 20, or 21 contiguous nucleobases that is 100% complementary to the nucleobase sequence of an equal length portion of the first modified oligonucleotide; and the nucleobase sequence of the second modified oligonucleotide is at least 80%, at least 85%, at least 90%, at least 95%, at least 98%, at least 99%, or 100% complementary to the nucleobase sequence of an equal length portion of the first modified oligonucleotide. In certain embodiments, the oligomeric duplex is an antisense compound.

In certain embodiments, an oligomeric duplex comprises a first oligomeric compound comprising a first modified oligonucleotide consisting of 19 to 25 linked nucleosides, wherein the nucleobase sequence of the first modified oligonucleotide comprises at least 10, at least 11, at least 12, at least 13, at least 14, at least 15, at least 16, at least 17, at least 18, at least 19, at least 20, or at least 21 contiguous nucleobases of the nucleobase sequence of any one of SEQ ID NO: 3-314, 627-782, 939-976, 1033-1038, 1254-1255, and 1258-1287; and a second oligomeric compound comprising a second modified oligonucleotide consisting of 16 to 24 linked nucleosides, wherein the nucleobase sequence of the second modified oligonucleotide comprises at least 9, at least 10, at least 11, at least 12, at least 13, at least 14, at least 15, at least 16, at least 17, at least 18, at least 19, or at least 20 contiguous nucleobases of the nucleobase sequence of any one of SEQ ID NO: 315-626, 783-938, 977-1016, 1027-1032, 1256-1257, and 1288-1290. In certain embodiments, the first oligomeric compound is an antisense compound, wherein the first modified oligonucleotide is an antisense oligonucleotide. In certain embodiments, the second oligomeric compound is a sense compound, wherein the second modified oligonucleotide is a sense oligonucleotide. In certain embodiments, the first modified oligonucleotide is an antisense RNAi oligonucleotide, and the second modified oligonucleotide is a sense RNAi oligonucleotide. In certain embodiments, the nucleobase sequence of the second modified oligonucleotide is at least 95% or 100% complementary to the nucleobase sequence of an equal length portion of the first modified oligonucleotide. In certain embodiments, the oligomeric duplex is an antisense compound.

In certain embodiments, an oligomeric duplex comprises a first oligomeric compound comprising a first modified oligonucleotide, wherein the first modified oligonucleotide consists of 23, 22 or 21 linked nucleosides and has nucleobase sequence comprising at least a 19-bp nucleobase sequence of any one of SEQ ID Nos: 3-314, 627-782, 939-976, 1033-1038, 1254-1255, and 1258-1287 having 0, 1, 2 or 3 nucleobases that are different from the corresponding nucleotide in any of SEQ ID Nos: 3-314, 627-782, 939-976, 1033-1038, 1254-1255, and 1258-1287; and a second oligomeric compound comprising a second modified oligonucleotide wherein the second modified oligonucleotide consists of 21 linked nucleosides and has a nucleobase sequence comprising at least a 16-bp nucleobase sequence of any one of SEQ ID Nos: 315-626, 783-938, 977-1016, 1027-1032, 1256-1257, and 1288-1290 having 0, 1, 2 or 3 nucleobases that are different from the corresponding nucleotide in any of SEQ ID Nos: 315-626, 783-938, 977-1016, 1027-1032, 1256-1257, and 1288-1290. In certain embodiments, the first oligomeric compound is an antisense compound, wherein the first modified oligonucleotide is an antisense oligonucleotide. In certain embodiments, the second oligomeric compound is a sense compound, wherein the second modified oligonucleotide is a sense oligonucleotide. In certain embodiments, the first modified oligonucleotide is an antisense RNAi oligonucleotide and the second modified oligonucleotide is a sense RNAi oligonucleotide. In certain embodiments, the nucleobase sequence of the second modified oligonucleotide is at least 95% or 100% complementary to the nucleobase sequence of an equal length portion of the first modified oligonucleotide. In certain embodiments, the oligomeric duplex is an antisense compound. In certain embodiments, the oligomeric duplex comprises one or two unpaired nucleosides at either or both ends, forming one or two overhang ends. In certain embodiments an overhang end is one or two nucleosides of the antisense oligonucleotide. In certain embodiments an overhang end is one or two 3′-nucleosides of the antisense oligonucleotide. In certain embodiments the last two 3′-nucleosides of the antisense oligonucleotide are overhang nucleosides not paired with the sense oligonucleotide. In certain embodiments the last one or two 3′-unpaired overhang nucleosides comprise an adenine nucleobase. In certain embodiments the last one or two 3′-unpaired overhang nucleosides comprise a hypoxanthine nucleobase. In certain embodiments the last one or two 3′-unpaired overhang nucleosides comprise a thymine nucleobase.

In certain embodiments, an oligomeric duplex comprises a first oligomeric compound comprising a first modified oligonucleotide, wherein the first modified oligonucleotide consists of 21, 20 or 19 linked nucleosides and has a nucleobase sequence comprising at least a 19-bp nucleobase sequence of any one of SEQ ID Nos: 3-314, 627-782, 939-976, 1033-1038, 1254-1255, and 1258-1287 having 0, 1, 2 or 3 mismatches with a sequence in a target PLN nucleic acid sequence; and a second oligomeric compound comprising a second modified oligonucleotide wherein the second modified oligonucleotide consists of 19 linked nucleosides, comprising at least a 19-bp nucleobase sequence of any one of SEQ ID Nos: 315-626, 783-938, 977-1016, 1027-1032, 1256-1257, and 1288-1290 having 0, 1, 2 or 3 mismatches to the first modified oligonucleotide. In certain embodiments, the first oligomeric compound is an antisense compound, wherein the first modified oligonucleotide is an antisense oligonucleotide. In certain embodiments, the second oligomeric compound is a sense compound, wherein the second modified oligonucleotide is a sense oligonucleotide. In certain embodiments, the first modified oligonucleotide is an antisense RNAi oligonucleotide and the second modified oligonucleotide is a sense RNAi oligonucleotide. In certain embodiments, the nucleobase sequence of the second modified oligonucleotide is at least 95% or 100% complementary to the nucleobase sequence of an equal length portion of the first modified oligonucleotide. In certain embodiments, the oligomeric duplex is an antisense compound. In certain embodiments, the oligomeric duplex comprises one or two unpaired nucleosides at either or both ends, forming one or two overhang ends. In certain embodiments an overhang end is one or two nucleosides of the antisense oligonucleotide. In certain embodiments an overhang end is one or two 3′-nucleosides of the antisense oligonucleotide. In certain embodiments the last two 3′-nucleosides of the antisense oligonucleotide are overhang nucleosides not paired with the sense oligonucleotide. In certain embodiments the last one or two 3′-unpaired overhang nucleosides comprise an adenine nucleobase. In certain embodiments the last one or two 3′-unpaired overhang nucleosides comprise a hypoxanthine nucleobase. In certain embodiments the last one or two 3′-unpaired overhang nucleosides comprise a thymine nucleobase.

In certain embodiments, an oligomeric duplex comprises a first oligomeric compound comprising a first modified oligonucleotide, wherein the first modified oligonucleotide consists of 23, 22 or 21 linked nucleosides and has a nucleobase sequence comprising at least a 19-bp nucleobase sequence of any one of SEQ ID Nos: 3-314, 627-782, 939-976, 1033-1038, 1254-1255, and 1258-1287; and a second oligomeric compound comprising a second modified oligonucleotide wherein the second modified oligonucleotide consists of 21 linked nucleosides and has a nucleobase sequence comprising at least a 19-bp nucleobase sequence of any one of SEQ ID Nos: 315-626, 783-938, 977-1016, 1027-1032, 1256-1257, and 1288-1290. In certain embodiments, the first oligomeric compound is an antisense compound, wherein the first modified oligonucleotide is an antisense oligonucleotide. In certain embodiments, the second oligomeric compound is a sense compound, wherein the second modified oligonucleotide is a sense oligonucleotide. In certain embodiments, the first modified oligonucleotide is an antisense RNAi oligonucleotide and the second modified oligonucleotide is a sense RNAi oligonucleotide. In certain embodiments, the nucleobase sequence of the second modified oligonucleotide is at least 95% or 100% complementary to the nucleobase sequence of an equal length portion of the first modified oligonucleotide. In certain embodiments, the oligomeric duplex is an antisense compound. In certain embodiments, the oligomeric duplex comprises one or two unpaired nucleosides at either or both ends, forming one or two overhang ends. In certain embodiments an overhang end is one or two nucleosides of the antisense oligonucleotide. In certain embodiments an overhang end is one or two 3′-nucleosides of the antisense oligonucleotide. In certain embodiments the last two 3′-nucleosides of the antisense oligonucleotide are overhang nucleosides not paired with the sense oligonucleotide. In certain embodiments the last one or two 3′-unpaired overhang nucleosides comprise an adenine nucleobase. In certain embodiments the last one or two 3′-unpaired overhang nucleosides comprise a hypoxanthine nucleobase. In certain embodiments the last one or two 3′-unpaired overhang nucleosides comprise a thymine nucleobase.

In certain embodiments, an oligomeric duplex comprises a first oligomeric compound comprising a first modified oligonucleotide, wherein the first modified oligonucleotide consists of 21 linked nucleosides and has a nucleobase sequence comprising at least a 19-bp nucleobase sequence of any one of SEQ ID Nos: 3-314, 627-782, 939-976, 1033-1038, 1254-1255, and 1258-1287; and a second oligomeric compound comprising a second modified oligonucleotide wherein the second modified oligonucleotide consists of 19 linked nucleosides and has a nucleobase sequence comprising at least a 16-bp nucleobase sequence of any one of SEQ ID Nos: 315-626, 783-938, 977-1016, 1027-1032, 1256-1257, and 1288-1290, wherein the nucleobase sequence of the second modified oligonucleotide is at least 90% complementary to the nucleobase sequence of an equal length portion of the first modified oligonucleotide. In certain embodiments, the first oligomeric compound is an antisense compound, wherein the first modified oligonucleotide is an antisense oligonucleotide. In certain embodiments, the second oligomeric compound is a sense compound, wherein the second modified oligonucleotide is a sense oligonucleotide. In certain embodiments, the first modified oligonucleotide is an antisense RNAi oligonucleotide and the second modified oligonucleotide is a sense RNAi oligonucleotide. In certain embodiments, the nucleobase sequence of the second modified oligonucleotide is at least 95% or 100% complementary to the nucleobase sequence of an equal length portion of the first modified oligonucleotide. In certain embodiments, the oligomeric duplex is an antisense compound. In certain embodiments, the oligomeric duplex comprises one or two unpaired nucleosides at either or both ends, forming one or two overhang ends. In certain embodiments an overhang end is one or two nucleosides of the antisense oligonucleotide. In certain embodiments an overhang end is one or two 3′-nucleosides of the antisense oligonucleotide. In certain embodiments the last two 3′-nucleosides of the antisense oligonucleotide are overhang nucleosides not paired with the sense oligonucleotide. In certain embodiments the last one or two 3′-unpaired overhang nucleosides comprise an adenine nucleobase. In certain embodiments the last one or two 3′-unpaired overhang nucleosides comprise a hypoxanthine nucleobase. In certain embodiments the last one or two 3′-unpaired overhang nucleosides comprise a thymine nucleobase.

In certain embodiments, an oligomeric duplex comprises a first oligomeric compound comprising a first modified oligonucleotide consisting of 19 to 25 linked nucleosides and a second oligomeric compound comprising a second modified oligonucleotide consisting of 16 to 24 linked nucleosides, wherein the nucleobase sequence of the first modified oligonucleotide and the nucleobase sequence of the second modified oligonucleotide each comprises at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases of any of the following pairs of nucleobase sequences recited in SEQ ID Nos: 939/978, 939/1001, 941/979, 941/1003, 942/980, 942/1004, 943/981, 943/1005, 944/982, 944/1006, 945/983, 945/1007, 946/984, 946/1008, 947/985, 947/1009, 940/977, 940/1002, 967/1011, 968/1012, 974/1013, 975/1014, 971/1015, 972/1016, 1254/1013, 1033/1039, 1034/1040, 1035/1041, 1036/1042, 1037/1043, 1038/1044, 1036/1288, and 1255/1041, 1265/1288, 1265/1042, 1259/1012, 1262/1013, 1268/1015, 1271/1016, 1264/1288, 1264/1042, 1258/1012, 1261/1013, 1267/1015, 1270/1046, 1260/1012, 1263/1013, 1266/1014, 1269/1015, 1272/1016, 1279/1288, 1273/1012, 1276/1013, 1282/1015, 1285/1016, 1280/1288, 1274/1012, 1277/1013, 1283/1015, 1286/1016, 1275/1012, 1278/1013, 1281/1014, 1284/1015, and 1287/1016, wherein the nucleobase sequence of the first modified oligonucleotide comprises a sequence of contiguous nucleobases of the nucleobase sequence of the first SEQ ID NO recited in the pair and the nucleobase sequence of the second modified oligonucleotide comprises a sequence of contiguous nucleobases of the nucleobase sequence of the second SEQ ID NO recited in the pair. In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide. In certain embodiments, the first modified oligonucleotide is an antisense RNAi oligonucleotide. In certain embodiments, the second oligomeric compound is a sense compound. In certain embodiments, the second modified oligonucleotide is a sense RNAi oligonucleotide. In certain embodiments, the oligomeric duplex comprises one or two unpaired nucleosides at either or both ends, forming one or two overhang ends. In certain embodiments an overhang end is one or two 3′-nucleosides of the antisense oligonucleotide. In certain embodiments the last two 3′-nucleosides of the antisense oligonucleotide are overhang nucleosides not paired with the sense oligonucleotide. In certain embodiments the last one or two 3′-unpaired overhang nucleosides comprise an adenine nucleobase. In certain embodiments the last one or two 3′-unpaired overhang nucleosides comprise a hypoxanthine nucleobase. In certain embodiments the last one or two 3′-unpaired overhang nucleosides comprise a thymine nucleobase.

In certain embodiments, an oligomeric duplex comprises a first oligomeric compound comprising a first modified oligonucleotide consisting of 21 or 23 linked nucleosides and a second oligomeric compound comprising a second modified oligonucleotide consisting of 19 or 21 linked nucleosides, wherein the nucleobase sequences of the first modified oligonucleotide and second modified oligonucleotide consist of any of the following pairs of nucleobase sequences recited in SEQ ID Nos: 939/978, 939/1001, 941/979, 941/1003, 942/980, 942/1004, 943/981, 943/1005, 944/982, 944/1006, 945/983, 945/1007, 946/984, 946/1008, 947/985, 947/1009, 940/977, 940/1002, 967/1011, 968/1012, 974/1013, 975/1014, 971/1015, 972/1016, 1254/1013, 1033/1039, 1034/1040, 1035/1041, 1036/1042, 1037/1043, 1038/1044, 1036/1288, and 1255/1041, 1265/1288, 1265/1042, 1259/1012, 1262/1013, 1268/1015, 1271/1016, 1264/1288, 1264/1042, 1258/1012, 1261/1013, 1267/1015, 1270/1046, 1260/1012, 1263/1013, 1266/1014, 1269/1015, 1272/1016, 1279/1288, 1273/1012, 1276/1013, 1282/1015, 1285/1016, 1280/1288, 1274/1012, 1277/1013, 1283/1015, 1286/1016, 1275/1012, 1278/1013, 1281/1014, 1284/1015, and 1287/1016, wherein the nucleobase sequence of the first modified oligonucleotide comprises the nucleobase sequence of the first SEQ ID NO recited in the pair and the nucleobase sequence of the second modified oligonucleotide comprises the nucleobase sequence of the second SEQ ID NO recited in the pair. In certain embodiments, the first oligomeric compound is an antisense compound. In certain embodiments, the first modified oligonucleotide is an antisense oligonucleotide. In certain embodiments, the second Oligomeric compound is a sense compound. In certain embodiments, the second modified oligonucleotide is a sense oligonucleotide. In certain embodiments, the first modified oligonucleotide is an antisense RNAi oligonucleotide. In certain embodiments, the second oligomeric compound is a sense compound. In certain embodiments, the second modified oligonucleotide is a sense RNAi oligonucleotide. In certain embodiments, the oligomeric duplex comprises one or two unpaired nucleosides at either or both ends, forming one or two overhang ends. In certain embodiments an overhang end is one or two 3′-nucleosides of the antisense oligonucleotide. In certain embodiments the last two 3′-nucleosides of the antisense oligonucleotide are overhang nucleosides not paired with the sense oligonucleotide. In certain embodiments the last one or two 3′-unpaired overhang nucleosides comprise an adenine nucleobase. In certain embodiments the last one or two 3′-unpaired overhang nucleosides comprise a hypoxanthine nucleobase. In certain embodiments the last one or two 3′-unpaired overhang nucleosides comprise a thymine nucleobase. In certain embodiments, the oligomeric duplex comprises one blunt end at either end, or two blunt ends.

In any of the oligomeric duplexes described herein, at least one nucleoside of the first modified oligonucleotide and/or the second modified oligonucleotide comprises a modified sugar moiety. Examples of suitable modified sugar moieties include, but are not limited to, a bicyclic sugar moiety, e.g., a modified furanosyl sugar moiety containing a 2′-4′ bridge selected from —O—CH₂—; and —O—CH(CH₃)—, and a non-bicyclic sugar moiety, e.g., a 2′-MOE sugar moiety, a 2′-F sugar moiety, a 2′-OMe sugar moiety, or a 2′-NMA sugar moiety. In certain embodiments, at least one nucleoside of the first modified oligonucleotide and/or the second modified oligonucleotide comprises a modified 2′-deoxy sugar moiety. In certain embodiments, at least 80%, at least 90%, or 100% of the nucleosides of the first modified oligonucleotide and/or the second modified oligonucleotide comprises a modified sugar moiety independently selected from 2′-F, 2′-MOE, 2′-OMe, and 2′-deoxy. In certain embodiments, at least 80%, at least 90%, or 100% of the nucleosides of the first modified oligonucleotide and the second modified oligonucleotide comprises a modified sugar moiety independently selected from 2′-F, 2′-MOE, 2′-OMe, and 2′-deoxy.

In certain embodiments, in an oligomeric duplex provided herein, at least one nucleoside of the first modified oligonucleotide and/or the second modified oligonucleotide comprises a sugar surrogate. Examples of suitable sugar surrogates include, but are not limited to, morpholino, hexitol nucleic acid (HNA), fluoro-hexitol nucleic acid (FHNA), the sugar surrogates of glycol nucleic acid (GNA), and unlocked nucleic acid (UNA). In certain embodiments, at least one nucleoside of the first modified oligonucleotide comprises a sugar surrogate, which is FHNA. In certain embodiments, at least 80%, at least 90%, or 100% of the nucleosides of the first modified oligonucleotide and/or the second modified oligonucleotide comprises a modified sugar moiety and/or sugar surrogate independently selected from 2′-F, 2′-MOE, 2′-OMe, 2′-deoxy, and FHNA. In certain embodiments, at least 80%, at least 90%, or 100% of the nucleosides of the first modified oligonucleotide and the second modified oligonucleotide comprises a modified sugar moiety and/or sugar surrogate independently selected from 2′-F, 2′-MOE, 2′-OMe, 2′-deoxy, and FHNA.

In certain embodiments, in an oligomeric duplex provided herein, at least one nucleoside of the first modified oligonucleotide and/or the second modified oligonucleotide comprises a modified sugar moiety and/or sugar surrogate. In certain embodiments, in an oligomeric duplex provided herein, a sugar moiety of the first modified oligonucleotide is modified, wherein the modified sugar moiety and/or sugar surrogate is selected from 2′-F, 2′-MOE, 2′-OMe, 2′-deoxy, and FHNA. In certain embodiments, in an oligomeric duplex provided herein, a sugar motif (from 5′ to 3′) of the first modified oligonucleotide is selected from yfyyyfyyyyyyyfyfyyyyyyy, efyyyfyyyyyyyfyfyyyyyee, efyyyyyyyyyyyfyyyyyyyee, efyyydyyyyyyyfyfyyyyyee, efyyydyyyyyyydydyyyyyyy, efyyydyyyyyyyfyfyyyyyyy, efyydydyyyyyyfyfyyyyyyy, efyyyfyyyyyyyfyyyyyyyee, efyyyyyyyyyyfyfyyyyyee, efyyyfyyyyyyyfyyyyyyyyy, efyyyyyyyyyyyfyfyyyyyyy, efyyyfyyyyyyyfyfyyyyyey, efyyyfyyyyyyyfydyyyyyee, efyyyfyyyyyyyfydyyyyyyy, efyydydyyyyyyfyfyyyyyee, efyyyfyyyyyyyfyfyyyee, e[FHNA]yydydyyyyfyfyfyyyyyyy, e[FHNA]yyyfyyyyyyyfyf yyyyyy, e[FHNA]yyyfyyyyyyyfyfyyyyyee, and e[FHNA]yyyyyyyyyyy[FHNA]yyyyyyyee, wherein each ‘y’ represents a 2′-OMe sugar moiety, each ‘f’ represents a 2′-F sugar moiety, each ‘[FHNA]’ represents a 3′-fluoro-hexitol sugar moiety, each ‘d’ represents a 2′-deoxy sugar moiety, and each ‘e’ represents a 2′-MOE sugar moiety. In certain embodiments, in an oligomeric duplex provided herein, a sugar moiety of the second modified oligonucleotide is modified, wherein the modified sugar moiety is selected from 2′-F, 2′-MOE, 2′-OMe, and 2′-deoxy. In certain embodiments, in an oligomeric duplex provided herein, a sugar motif (from 5′ to 3′) of the second modified oligonucleotide is selected from among: yyyyyyfyfffyyyyyyyyyy, yyyyyyyyyyyyyyyyyyyyy, yyyyyyyyyffyyyyyyyyyy, yyyyyyfyfyfyyyyyyyyyy, yyyyyyyyyydyyyyyyyyyy, eeyyyyfyfffyyyyyyyyee, eeyyyyyyyffyyyyyyyyee, and eeyyyyfyfyfyyyyyyyyee, wherein each ‘y’ represents a 2′-OMe sugar moiety, each ‘f’ represents a 2′-F sugar moiety, each ‘d’ represents a 2′-deoxy sugar moiety, and each ‘e’ represents a 2′-MOE sugar moiety.

In certain embodiments, in an oligomeric duplex provided herein, at least one internucleoside linkage of the first modified oligonucleotide and/or the second modified oligonucleotide comprises a modified internucleoside linkage. In certain embodiments, the modified internucleoside linkage is a phosphorothioate internucleoside linkage. In certain embodiments, at least one of the first, second, or third internucleoside linkages from the 5′ end and/or the 3′ end of the first modified oligonucleotide comprises a phosphorothioate linkage. In certain embodiments, at least one of the first, second, or third internucleoside linkages from the 5′ end and/or the 3′ end of the second modified oligonucleotide comprises a phosphorothioate linkage. In certain embodiments, the modified internucleoside linkage is a mesyl phosphoramidate internucleoside linkage. In certain embodiments, at least one of the internucleoside linkages of the first modified oligonucleotide comprises a mesyl phosphoramidate internucleoside linkage. In certain embodiments, at least one of the internucleoside linkages of the second modified oligonucleotide comprises a mesyl phosphoramidate internucleoside linkage.

In certain embodiments, in an oligomeric duplex provided herein, each internucleoside linkage of the first modified oligonucleotide is independently selected from a phosphodiester, a phosphorothioate, or a mesyl phosphoramidate internucleoside linkage, and each internucleoside linkage of the second modified oligonucleotide is independently selected from a phosphodiester, a phosphorothioate, or a mesyl phosphoramidate internucleoside linkage.

In certain embodiments, in an oligomeric duplex provided herein, each internucleoside linkage of the first modified oligonucleotide is independently selected from a phosphodiester or a phosphorothioate internucleoside linkage and each internucleoside linkage of the second modified oligonucleotide is independently selected from a phosphodiester or a phosphorothioate internucleoside linkage.

In certain embodiments, in an oligomeric duplex provided herein, at least one internucleoside linkage of the first modified oligonucleotide (e.g., antisense oligonucleotide) is a modified internucleoside linkage. In certain embodiments, in an oligomeric duplex provided herein, an internucleoside linkage of the first modified oligonucleotide is modified, wherein the 5′-most internucleoside linkage (i.e., linking the first nucleoside from the 5′-end to the second nucleoside from the 5′-end) is modified. In certain embodiments, in an oligomeric duplex provided herein, the internucleoside linkage motif (from 5′ to 3′) of the first modified oligonucleotide is selected from 5′-ssooooooooooooooooooss-3′, 5′-ssooooooooooooooooss-3′, 5′-ssooosooooooooooooooss-3′, 5′-ssoososoooooooooooooss-3′, wherein each “s” is a phosphorothioate internucleoside linkage and each “o” is a phosphodiester internucleoside linkage. In certain embodiments, in an oligomeric duplex provided herein, an internucleoside linkage of the second modified oligonucleotide is modified, wherein the 5′-most internucleoside linkage (i.e., linking the first nucleoside from the 5′-end to the second nucleoside from the 5′-end) is modified. In certain embodiments, in an oligomeric duplex provided herein, the internucleoside linkage motif (from 5′ to 3′) of the second modified oligonucleotide is selected from (from 5′ to 3′) of: ssooooooooooooooooss, ssoooooozozoooooooss, ssooooooosooooooooss, ssooooooooooooooss, wherein each ‘o’ represents a phosphodiester internucleoside linkage and each ‘s’ represents a phosphorothioate internucleoside linkage and each ‘z’ represents a mesyl phosphoramidate internucleoside linkage. In certain embodiments, the two 5′-most internucleoside linkages of a first modified oligonucleotide and/or second modified oligonucleotide of an oligomeric duplex are modified. In certain embodiments, the first one or 2 internucleoside linkages from the 3′-end of a first modified oligonucleotide and/or second modified oligonucleotide of an oligomeric duplex are modified. In certain embodiments, the two 5′-most internucleoside linkages of a first modified oligonucleotide and/or second modified oligonucleotide of an oligomeric duplex are modified and the first two internucleoside linkages from the 3′-end of the first modified oligonucleotide and/or second modified oligonucleotide are modified. In certain embodiments, the modified internucleoside linkage is a phosphorothioate linkage. In certain embodiments, the modified internucleoside linkage is a mesyl phosphoramidate linkage.

In certain embodiments, in an oligomeric duplex provided herein, at least one nucleobase of the first modified oligonucleotide and/or at least one nucleobase of the second modified oligonucleotide is a modified nucleobase. In certain embodiments, the modified nucleobase is 5-methylcytosine. In certain embodiments, the modified nucleobase is hypoxanthine.

In certain embodiments, in an oligomeric duplex provided herein, the first modified oligonucleotide is attached to a terminal group comprising a stabilized phosphate group attached to the 5′ position of the 5′-most nucleoside. In certain embodiments, the stabilized phosphate group comprises a cyclopropyl phosphonate or an (E)-vinyl phosphonate. In certain particular embodiments, the stabilized phosphate group is an (E)-vinyl phosphonate.

In certain embodiments, in an oligomeric duplex provided herein, the first modified oligonucleotide is attached to a conjugate group. In certain embodiments, the conjugate group comprises a conjugate linker and a conjugate moiety. In certain embodiments, the conjugate group is attached to the first modified oligonucleotide at the 5′-end of the first modified oligonucleotide. In certain embodiments, the conjugate group is attached to the first modified oligonucleotide at the 3′-end of the modified oligonucleotide. In certain embodiments, the conjugate group is attached to the first modified oligonucleotide at an internal position. In certain embodiments, the conjugate group is attached to the first modified oligonucleotide through a 2′-modification of a furanosyl sugar moiety. In certain embodiments, the conjugate group is attached to the first modified oligonucleotide through a modified internucleoside linkage. In certain embodiments, the conjugate group comprises N-acetyl galactosamine. In certain embodiments, the conjugate group comprises a cell-targeting moiety having an affinity for transferrin receptor (TfR), TfR1, also known as CD71, TFRC. In certain embodiments, the conjugate group comprises a TfR1 targeting peptide or polypeptide, targeting protein, or targeting low molecular weight molecule. In certain embodiments the conjugate group comprises a TfR1 targeting antibody or fragment thereof. In certain embodiments, the conjugate group comprises a protein or peptide capable of binding TfR1. In certain embodiments, the conjugate group comprises an aptamer capable of binding TfR1. In certain embodiments the conjugate group comprises a bicycle ligand that binds a transferrin receptor. In certain embodiments a conjugate group comprises BCY17901 as further described herein. In certain embodiments, a conjugate group comprises a moiety selected from any of a C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C17 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl. In certain embodiments, a conjugate group comprises a moiety selected from any of C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain optionally has one or more unsaturated bonds.

In any of the oligomeric duplexes described herein, the second modified oligonucleotide optionally is attached to a conjugate group. In certain embodiments, the conjugate group comprises a conjugate linker and a conjugate moiety. In certain embodiments, the conjugate group is attached to the second modified oligonucleotide at the 5′-end of the second modified oligonucleotide. In certain embodiments, the conjugate group is attached to the second modified oligonucleotide at the 3′-end of the modified oligonucleotide. In certain embodiments, the conjugate group is attached to the second modified oligonucleotide at an internal position. In certain embodiments, the conjugate group is attached to the second modified oligonucleotide through a 2′-modification of a furanosyl sugar moiety. In certain embodiments, the conjugate group is attached to the second modified oligonucleotide through a modified internucleoside linkage. In certain embodiments, the conjugate group comprises N-acetyl galactosamine. In certain embodiments, the conjugate group comprises a cell-targeting moiety having an affinity for transferrin receptor (TfR), TfR1, also known as CD71, TFRC. In certain embodiments, the conjugate group comprises a TfR1 targeting peptide or polypeptide, targeting protein, or targeting low molecular weight molecule. In certain embodiments the conjugate group comprises an anti-TfR1 targeting antibody or fragment thereof. In certain embodiments, the conjugate group comprises a protein or peptide capable of binding TfR1. In certain embodiments, the conjugate group comprises an aptamer capable of binding TfR1. In certain embodiments the conjugate group comprises a bicycle ligand that binds a transferrin receptor. In certain embodiments the conjugate group comprises BCY17901 as further described herein. In certain embodiments, a conjugate group comprises a moiety selected from any of a C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C17 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl. In certain embodiments, a conjugate group comprises a moiety selected from any of C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain optionally has one or more unsaturated bonds.

In certain embodiments, an oligomeric compound comprises an antisense compound, which comprises an oligomeric duplex described herein. In certain embodiments, an antisense compound, which comprises an oligomeric duplex described herein, is an RNAi agent capable of reducing the amount of PLN RNA through the activation of RISC/Ago2.

In certain embodiments, an oligomeric compound comprises at least two oligomeric duplexes linked together. In certain embodiments, an oligomeric compound comprises two oligomeric duplexes wherein at least one oligomeric duplex comprises an oligonucleotide comprising a portion having a nucleobase sequence complementary to a nucleobase sequence in a PLN nucleic acid (i.e., is targeted to PLN RNA) as described herein. In certain embodiments, an oligomeric compound comprises two or more of the same oligomeric duplex, which is any of the oligomeric duplexes described herein. In certain embodiments, the two or more oligomeric duplexes are covalently linked together. In certain embodiments, the second modified oligonucleotides of the two or more oligomeric duplexes are covalently linked together. In certain embodiments, the second modified oligonucleotides of two or more oligomeric duplexes are covalently linked together at their 3′ ends. In certain embodiments, the second modified oligonucleotides of two or more oligomeric duplexes are covalently linked together at the 3′ end of one to the 5′ end of the other. In certain embodiments, the two or more oligomeric duplexes are covalently linked together by a glycol linker, e.g., a tetraethylene glycol linker. A structure of oligomeric duplexes covalently linked by a glycol linker is described in, e.g., Alterman, et al., Nature Biotech., 37:844-894, 2019. In some embodiments, an oligomeric compound comprises two or more oligomeric duplexes linked, e.g., covalently linked, together in a branched structure, e.g., a di-branched, tri-branched or tetra-branched structure (see, e.g., WO2022/256565). In some such embodiments, the structure contains a linker (e.g., one or more subunits of an ethylene glycol, alkyl, carbohydrate, block copolymer, peptide, ester, amide, carbamate, triazole) and optionally one or more branch point moieties (e.g., phosphoroamidite, tosylated solketal, 1,3-diaminopropanol, pentaerythritol).

In certain embodiments, an oligomeric duplex comprises two or more regions each of which has a nucleobase sequence complementary to the nucleobase sequence of a different target region of the same nucleic acid target (PLN nucleic acid), or one of which has a nucleobase sequence complementary to the nucleobase sequence of a target region of a PLN nucleic acid (e.g., a PLN RNA such as described herein) and the other having a nucleobase sequence complementary to the nucleobase sequence of a target region of a different nucleic acid target (i.e., other than a PLN nucleic acid target). In some embodiments, the nucleobase sequence(s) complementary to a target region of a PLN nucleic acid targets a PLN nucleic acid region as described herein and/or comprises at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases of the nucleobase sequence of any one of SEQ ID NOs: 3-314, 627-782, 939-976, 1033-1038, 1254-1255, or 1258-1287. In certain embodiments, such an oligomeric duplex comprises a first modified oligonucleotide comprising (1) a first region having a nucleobase sequence complementary to a first sequence in a PLN target nucleic acid and (2) a second region having a nucleobase sequence complementary to (a) a different (i.e., second) sequence in a PLN target nucleic acid, or (b) a sequence in a target nucleic acid other than a PLN nucleic acid. In some such embodiments, the second modified oligonucleotide of the oligomeric duplex comprises (1) a region having a nucleobase sequence complementary to the first region of the first modified oligonucleotide and (2) a region having a nucleobase sequence complementary to the second region of the first modified oligonucleotide. In certain embodiments, an oligomeric duplex comprises a first modified oligonucleotide comprising a first region having a nucleobase sequence complementary to a first sequence in a PLN target nucleic acid and a second modified oligonucleotide comprising a second region having a nucleobase sequence complementary to: (a) the nucleobase sequence of a different (i.e., second) sequence in a PLN target nucleic acid, or (b) a sequence in a target nucleic acid other than a PLN nucleic acid. In some such embodiments, the first modified oligonucleotide further comprises a region having a nucleobase sequence complementary to the nucleobase sequence of the second region in the second modified oligonucleotide. In some such embodiments, the second modified oligonucleotide further comprises a region having a nucleobase sequence complementary to the nucleobase sequence of the first region in the first modified oligonucleotide (see, e.g., PCT International Patent Application Publication WO2020/065602).

### III. Conjugates

In certain embodiments, provided herein are oligomeric compounds comprising one or more modified oligonucleotide and one or more conjugate groups. In certain embodiments, an oligomeric compound optionally further comprises one or more terminal groups. In certain embodiments, oligomeric compounds comprise an oligonucleotide, a cell-targeting moiety, and a conjugate linker. In certain embodiments, oligomeric compounds comprise an oligonucleotide, a transferrin receptor ligand, and a conjugate linker. In certain embodiments, oligomeric compounds comprise an oligonucleotide, a bicycle ligand, and a conjugate linker. In certain embodiments, oligomeric compounds comprise an oligonucleotide, a peptide or polypeptide, a conjugate linker, and optionally N-terminal or C-terminal modifications to the peptide or polypeptide. In certain embodiments, oligomeric compounds comprise an oligonucleotide, two or more peptides or polypeptides, a branching group, a conjugate linker, and optionally N-terminal or C-terminal modifications to the peptides or polypeptides. In certain embodiments, a conjugate linker connects a peptide or polypeptide and/or a bicycle ligand to an oligonucleotide.

In certain embodiments, the N-terminus of a bicycle ligand is covalently connected to a conjugate linker, and the conjugate linker is covalently connected to the 3′ end of an oligonucleotide. In certain embodiments, the C-terminus of a bicycle ligand is covalently connected to a conjugate linker, and the conjugate linker is covalently connected to the 3′ end of an oligonucleotide. In certain embodiments, an internal amino acid of a bicycle ligand is covalently connected to a conjugate linker, and the conjugate linker is covalently connected to the 3′ end of an oligonucleotide. In certain embodiments, the N-terminus of a bicycle ligand is covalently connected to a conjugate linker, and the conjugate linker is covalently connected to the 5′ end of an oligonucleotide. In certain embodiments, the C-terminus of a bicycle ligand is covalently connected to a conjugate linker, and the conjugate linker is covalently connected to the 5′ end of an oligonucleotide. In certain embodiments, an internal amino acid of a bicycle ligand is covalently connected to a conjugate linker, and the conjugate linker is covalently connected to the 5′ end of an oligonucleotide. In certain embodiments, the N-terminus of a bicycle ligand is covalently connected to a conjugate linker, and the conjugate linker is covalently connected at an internal position of an oligonucleotide. In certain embodiments, the C-terminus of a bicycle ligand is covalently connected to a conjugate linker, and the conjugate linker is covalently connected at an internal position of an oligonucleotide. In certain embodiments, an internal amino acid of a bicycle ligand is covalently connected to a conjugate linker, and the conjugate linker is covalently connected at an internal position of an oligonucleotide. In certain embodiments, an internal position of an oligonucleotide is a 2′-position of a modified sugar moiety of a nucleoside within the internal region of an oligonucleotide that is not the 5′ terminal nucleoside or the 3′ terminal nucleoside. In certain embodiments, an internal position of an oligonucleotide is a modified internucleoside linkage of the oligonucleotide.

### A. Conjugate Groups

In certain embodiments, a conjugate moiety modifies one or more properties of an attached oligonucleotide, including but not limited to pharmacodynamics, pharmacokinetics, stability, binding, absorption, tissue distribution, cellular distribution, cellular uptake, charge and clearance. In certain embodiments, a conjugate moiety imparts a new property on the attached oligonucleotide.

In certain embodiments, a conjugate group comprises a conjugate moiety and a conjugate linker. In certain embodiments, a conjugate moiety comprises or consists of a cell-targeting moiety. In certain embodiments, a cell-targeting moiety is capable of binding the cell-surface receptor or the cell-surface moiety. In certain embodiments, a compound comprising a cell-targeting moiety is capable of being internalized when it interacts with or binds the cell-surface receptor or the cell-surface moiety. In certain embodiments, a cell-targeting moiety comprises a bicyclic peptide or polypeptide or a bicycle ligand. In certain embodiments, a cell-targeting moiety consists of a bicyclic peptide or polypeptide or a bicycle ligand.

In certain embodiments, a bicycle ligand comprises a bicyclic peptide ligand including those previously described in International Patent Application Publication No. WO2022/101633, which is incorporated herein by reference, and those previously described in International Patent Application Publication No. WO2023/056388, which is hereby incorporated by reference.

In certain embodiments, a bicycle ligand comprises a peptide or polypeptide or peptidomimetic comprising at least three reactive groups, separated by at least two loop sequences, and a molecular scaffold. A molecular scaffold is a chemical group which forms covalent bonds with reactive groups of a peptide. Through such bonding, at least two peptide loops are formed on the molecular scaffold. In certain embodiments, the molecular scaffold is 1,1′,1″-(1,3,5-triazinane-1,3,5-triyl)triprop-2-en-1-one (TATA). In certain embodiments, the molecular scaffold is 1,1′,1″-(1,3,5-triazinane-1,3,5-triyl)tris(2-bromoethanone) (TATB).

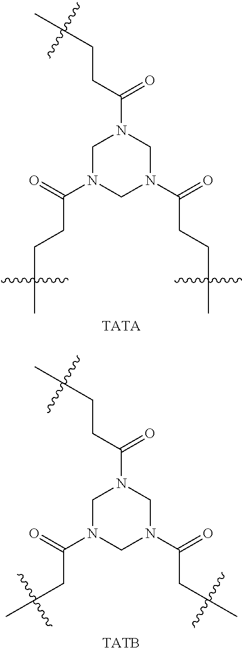 [SMILES: *CCC(=O)N1CN(C(=O)CC*)CN(C(=O)CC*)C1 ; "TATA" ; *CCC(=O)N1CN(C(=O)C*)CN(C(=O)C*)C1]

In certain embodiments, a bicycle ligand peptide or peptidomimetic comprises natural amino acids, non-natural amino acids and/or amino acid mimetics. In certain embodiments, reactive groups of a bicycle ligand peptide are cysteines. In certain embodiments, loop sequences comprise 2, 3, 4, 5, 6, 7, 8, or 9 amino acids. In certain embodiments, a bicycle ligand peptide comprises three cysteine residues separated by two loop sequences, the first of which consists of 2 amino acids and the second of which consists of 9 amino acids. In certain embodiments, a bicycle ligand peptide comprises three cysteine residues separated by two loop sequences both of which consist of 6 amino acids. In certain embodiments, a bicycle ligand peptide comprises three cysteine residues separated by two loop sequences the first of which consists of 3 amino acids and the second of which consists of 8 amino acids. In certain embodiments, a bicycle ligand peptide comprises three cysteine residues separated by two loop sequences the first of which consists of 7 amino acids and the second of which consists of 3 amino acids.

In certain embodiments, a bicycle ligand comprises an amino acid sequence selected from:

CᵢXXCᵢᵢXXXXXXXXXCᵢᵢᵢ,
CᵢXXXXXXCᵢᵢXXXXXXCᵢᵢᵢ,
CᵢXXXCᵢᵢXXXXXXXXCᵢᵢᵢ,
or
CᵢXXXXXXCᵢᵢXXXCᵢᵢᵢ;

wherein C₁, Cᵢᵢ and Cᵢᵢᵢ represent first, second and third cysteine residues, respectively, and each “X” represents an independently selected natural or non-natural amino acid, or a pharmaceutically acceptable salt thereof. In certain embodiments, a bicycle ligand further comprises an N-terminal extension and/or a C-terminal extension.

In certain embodiments, a bicycle ligand comprises an amino acid sequence at least 80%, 85%, 90%, 95%, or 100% identical to any of the following sequences:

(SEQ ID NO: 1062)
CᵢSPDAHLGCᵢᵢISYCᵢᵢᵢ;
(SEQ ID NO: 1063)
CᵢSPDAYLGCᵢᵢISYCᵢᵢᵢ;
(SEQ ID NO: 1064)
CᵢP[HyP]DAYLGCᵢᵢISYCᵢᵢᵢ;
(SEQ ID NO: 1065)
CᵢS[HyP]DAHLGCᵢᵢISYCᵢᵢᵢ;
(SEQ ID NO: 1066)
CᵢS[Aze]DAHLGCᵢᵢISYCᵢᵢᵢ;
(SEQ ID NO: 1067)
CᵢP[HyP]DAYLGCᵢᵢ[tBuGly]SYCᵢᵢᵢ;
(SEQ ID NO: 1068)
Cᵢ[K(N₃)]PDAHLGCᵢᵢISYCᵢᵢᵢ;
(SEQ ID NO: 1069)
CᵢS[K(N₃)]DAHLGCᵢᵢISYCᵢᵢᵢ;
or
(SEQ ID NO: 1070)
CᵢSPD[K(N₃)]HLGCᵢᵢISYCᵢᵢᵢ;

Cᵢ, Cᵢᵢ and Cᵢᵢᵢ represent first, second and third cysteines, respectively; [HyP] represents 4-trans-hydroxy-L-proline; [Aze] represents azetidine; [tBuGly] represents t-butyl glycine; and [K(N₃)] represents 6-azido lysine. In certain embodiments, a bicycle ligand further comprises an N-terminal extension and/or a C-terminal extension.

In certain embodiments, a bicycle ligand comprises an amino acid sequence of: CᵢXXDXXXGCᵢᵢISYCᵢᵢᵢ (SEQ ID NO: 1026); wherein each X is independently selected from natural or non-natural amino acid, or a pharmaceutically acceptable salt thereof. In certain embodiments, a bicycle ligand further comprises an N-terminal extension and/or a C-terminal extension.

In certain embodiments, an oligomeric compound comprises two or more bicyclic ligands attached through a conjugate linker including a bivalent linker including a branching group. In certain embodiments, a bivalent linker comprises one or more PEG repeats. In certain embodiments, the bivalent linker is shown in the structure below:

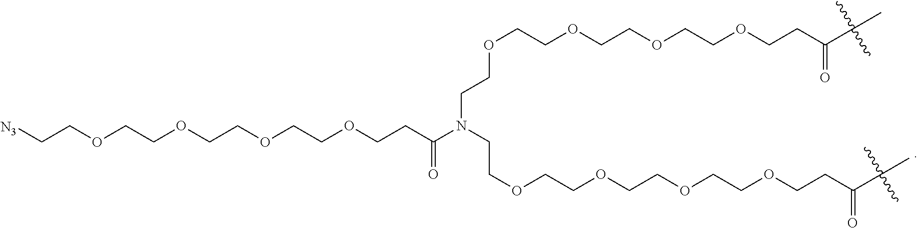 [SMILES: *C(=O)CCOCCOCCOCCOCCN(CCOCCOCCOCCOCCC(*)=O)C(=O)CCOCCOCCOCCOCCN=[N+]=[N-]]

In certain embodiments, a bicycle ligand is capable of interacting with a cell surface receptor on a cell. In certain embodiments, a bicycle ligand is capable of interacting with a cell surface moiety on a cell. In certain embodiments, a bicycle ligand is capable of binding a cell surface receptor on a cell. In certain embodiments, a bicycle ligand is capable of binding a cell surface moiety on a cell. In certain embodiments, a bicycle ligand is capable of being internalized by the cell when it interacts with and/or binds a cell surface receptor and/or cell surface moiety. In certain embodiments, a cell surface receptor is not expressed ubiquitously (e.g., the cell surface receptor is undetectable in at least one tissue of a human subject), and a bicycle ligand selectively delivers an oligonucleotide, oligomeric compound or oligomeric duplex to a tissue of interest or a cell of interest. By way of non-limiting example, tissue of interest may be any one or more of brain, spinal cord, retina, heart, kidney, liver, lung, skeletal muscle, cardiac muscle, smooth muscle, adipose, white adipose, brown adipose, spleen, bone, intestine, colon, testes, breast, ovary, placenta, uterus, bladder, pancreas, pituitary, prostate, skin, adrenal gland, and thyroid. By way of non-limiting example, the cell of interest may be any one or more of a myocyte, adipocyte, hepatocyte, cardiomyocyte, vascular smooth muscle cell, endothelial cell, neuron, blood cell, macrophage, lymphocyte, cancer cell, and immune cell.

In certain embodiments, a bicycle ligand is capable of interacting with or binding a cell surface receptor. In certain embodiments, the cell surface receptor is capable of internalizing the bicycle ligand. In certain embodiments, the cell surface receptor is capable of internalizing an oligonucleotide, oligomeric compound or oligomeric duplex connected to the bicycle ligand via a conjugate linker. In certain embodiments, the cell surface receptor is a human transferrin receptor.

In certain embodiments, a bicycle ligand is represented by the formula [B]ₙ—[Z₁]-[J]ₘ—[Zᵢᵢ]—[O]ₒ—[Zᵢᵢᵢ]—[U]ₚ, wherein:
Zᵢ, Zᵢᵢ, and Zᵢᵢᵢ are the first, second, and third amino acids comprising a reactive group;
each B, J, O, and U is independently selected amino acids or amino acid mimetics;
n is from 0 to 5;
m is from 3 to 7;
o is from 3 to 7;
p is from 0 to 5;
wherein the sum of m+o is less than 12.

In particular embodiments, m is 7 and o is 3.

In certain embodiments, a bicycle ligand comprises the following structure,

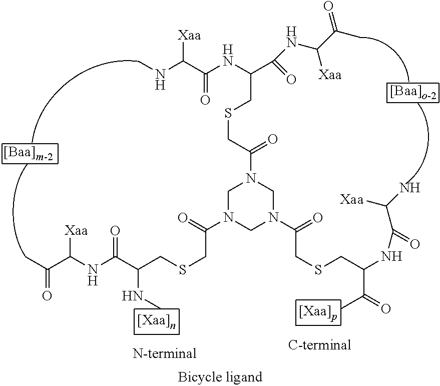

wherein each Xaa is an independently selected amino acid side chain;
each Baa is an independently selected amino acid or amino acid mimetic;
n is from 0 to 5;
m is from 3 to 7;
o is from 3 to 7;
p is from 0 to 5;
wherein the sum of m+o is less than 12.

In particular embodiments, m is 7 and o is 3.

In certain embodiments, the conjugate linker is attached through the N-terminus, the C-terminus, or through one of the loop amino acids.

In certain embodiments, a bicycle ligand comprises the formula [Zᵢ]-[J]ₘ—[Zᵢᵢ]—[O]ₒ—[Zᵢᵢᵢ]. In certain embodiments, a bicycle ligand comprises the following structure:

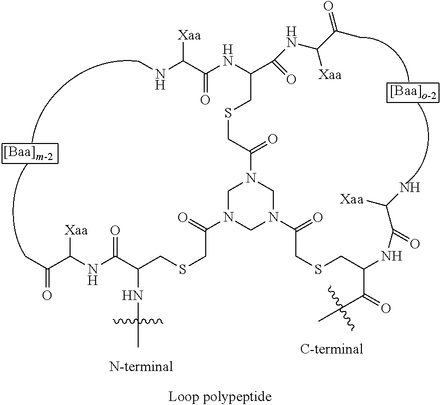

wherein each Xaa is an independently selected amino acid side chain, each Baa is an independently selected amino acid or amino acid mimetic;
m is from 3 to 7 and;
o is from 3 to 7.

In certain embodiments, a bicycle ligand further comprises an N-terminal extension and/or a C-terminal extension.

In certain embodiments, a conjugate linker is attached through the N-terminus, the C-terminus, or through one of the loop amino acids.

In certain embodiments, a bicycle ligand comprises or consists of a sequence selected from Table D:

TABLE D
Bicycle ligands

| Bicycle | N-terminal | | |
|---|---|---|---|
| Number | Mod. | Sequence | SEQ ID NO: |
| BCY13983 | none | ACSADDWLGCISWCA | 1071 |
| BCY14474 | none | ACSADDWLGCISWCA[Sar]₆[K-F1] | 1072 |
| BCY13986 | none | ACSSDAYLGCISWCA | 1073 |
| BCY14475 | none | ACSSDAYLGCISWCA[Sar]₆[K-F1] | 1074 |
| BCY15466 | none | ACPPDAHLGCISWCA | 1075 |
| BCY15889 | Ac | CPPDAHLGCISWC | 1076 |
| BCY15467 | none | ACPQDAYLGCISWCA | 1077 |
| BCY15890 | Ac | CPQDAYLGCISWC | 1078 |
| BCY13989 | none | ACPPDSWQGCISYCA | 1079 |
| BCY14476 | none | ACPPDSWQGCISYCA[Sar]₆[K-F1] | 1080 |
| BCY15468 | none | ACSPDAHLGCISYCA | 1081 |
| BCY15768 | none | ACSPDAHLGCISYCA[Sar]₆[K-F1] | 1082 |
| BCY15934 | none | CSPDAHLGCISYC[Sar]₆[K-F1] | 1083 |
| BCY15937 | Ac | CSPDAHLGCISYCA[Sar]₆[K-F1] | 1084 |
| BCY15938 | Ac | CSPDAHLGCISYC[Sar]₆[K-F1] | 1085 |
| BCY15940 | none | [Fl-G][Sar]₅ACSPDAHLGCISYCA | 1086 |
| BCY18030 | none | N[1Nal]NCSPDAHLGCISYC | 1087 |
| BCY18039 | Ac | CSPDAHLGCISYCE[Pip]W | 1088 |
| BCY17994 | Ac | CSPDAHLGCISYCEPW | 1089 |
| BCY18029 | none | NWNCSPDAHLGCISYC | 1090 |
| BCY17109 | none | NWNCSPDAHLGCISYCA | 1091 |
| BCY18037 | Ac | CSPDAHLGCISYCE[Aze]W | 1092 |
| BCY17992 | Ac | NWNCSPDAHLGCISYC | 1093 |
| BCY18038 | Ac | CSPDAHLGCISYCE[dP]W | 1094 |
| BCY18034 | Ac | N[1Nal]NCSPDAHLGCISYC | 1095 |
| BCY18031 | none | N[dW]NCSPDAHLGCISYC | 1096 |
| BCY18035 | Ac | N[dW]NCSPDAHLGCISYC | 1097 |
| BCY17110 | none | HWMCSPDAHLGCISYCA | 1098 |
| BCY17115 | none | ACSPDAHLGCISYCPHP | 1099 |
| BCY17114 | none | ACSPDAHLGCISYCEPW | 1100 |
| BCY17112 | none | NEVCSPDAHLGCISYCA | 1101 |
| BCY17120 | none | ACSPDAHLGCISYCPIVH | 1102 |
| BCY15891 | Ac | CSPDAHLGCISYC | 1103 |
| BCY17111 | none | HTSCSPDAHLGCISYCA | 1104 |
| BCY18036 | Ac | N[NMeTrp]NCSPDAHLGCISYC | 1105 |
| BCY18032 | none | N[NMeTrp]NCSPDAHLGCISYC | 1106 |
| BCY15939 | Ac | ACSPDAHLGCISYCA | 1107 |
| BCY17119 | none | ACSPDAHLGCISYCEHQE | 1108 |
| BCY17113 | none | ESFCSPDAHLGCISYCA | 1109 |
| BCY17870 | none | NWNCSPDAHLGCISYC[K(N₃)] | 1110 |
| BCY17871 | Ac | NWNCSPDAHLGCISYC[K(N₃)] | 1111 |
| BCY17872 | AzPro | NWNCSPDAHLGCISYC | 1112 |
| BCY17873 | Ac | CSPDAHLGCISYCEPW[K(N₃)] | 1113 |
| BCY17874 | AzPro | CSPDAHLGCISYCEPW | 1114 |
| BCY17868 | Ac | CSPDAHLGCISYC[K(N₃)] | 1115 |
| BCY17869 | AzPro | CSPDAHLGCISYC | 1116 |
| BCY17882 | Ac | N[dY]NCSPDAHLGCISYC[K(N₃)] | 1117 |
| BCY17890 | Ac | CSPDAHLGCISYCE-[dP]W[K(N₃)] | 1118 |
| BCY17892 | Ac | CSPDAHLGCISYCE-[Aze]W[K(N₃)] | 1119 |
| BCY17894 | Ac | CSPDAHLGCISYCE-[Pip]W[K(N₃)] | 1120 |
| BCY17906 | Ac | CSPDAHLGCISYC[K(N₃)(PYA-maleimide)] | 1121 |
| BCY19405 | Ac | CSPDAHLGCISYCEPW[PEG10][K(N₃)] | 1122 |
| BCY19406 | Ac | CSPDAHLGCISYCEPW[PEG24][K(N₃)] | 1123 |
| BCY19407 | Ac | CSPDAHLGCISYCEPWGGSGGS[K(N₃)] | 1124 |
| BCY15469 | none | ACPGDAHLGCISYCA | 1125 |
| BCY15892 | Ac | CPGDAHLGCISYC | 1126 |
| BCY15470 | none | ACPPDSHLGCISYCA | 1127 |
| BCY15893 | Ac | CPPDSHLGCISYC | 1128 |
| BCY15471 | none | ACSADDWLGCISYCA | 1129 |
| BCY15894 | Ac | CSADDWLGCISYC | 1130 |
| BCY17991 | Ac | CP[HyP]DAYLGC[tBuGly]SYC | 1131 |
| BCY17995 | Ac | CP[HyP]DAYLGC[tBuGly]SYCEPW | 1132 |
| BCY17993 | Ac | NWNCP[HyP]DAYLGC[tBuGly]SYC | 1133 |
| BCY16754 | none | ACP[HyP]DAYLGC[tBuGly]SYCA | 1134 |
| BCY18033 | none | NWNCP[HyP]DAYLGC[tBuGly]SYC | 1135 |
| BCY17896 | Ac | CP[HyP]DAYLGC[tBuGly]SYC[K(N₃)] | 1136 |
| BCY17899 | Ac | NWNCP[HyP]DAYLGC[tBuGly]SYC[K(N₃)] | 1137 |
| BCY17901 | Ac | CP[HyP]DAYLGC[tBuGly]SYCEPW[K(N₃)] | 1253 |
| BCY17990 | Ac | CP[HyP]DAYLGCISYC | 1138 |
| BCY17875 | Ac | CP[HyP]DAYLGCISYC[K(N₃)] | 1139 |
| BCY17876 | AzPro | CP[HyP]DAYLGCISYC | 1140 |
| BCY17989 | Ac | CS[HyP]DAHLGCISYC | 1141 |
| BCY16047 | none | ACS[HyP]DAHLGCISYCA | 1142 |
| BCY17877 | Ac | CS[HyP]DAHLGCISYC[K(N₃)] | 1143 |
| BCY17878 | AzPro | CS[HyP]DAHLGCISYC | 1144 |
| BCY16962 | none | ACP[Aib]DAHLGC[tBuGly]SYCA | 1145 |
| BCY17117 | none | TYMNCPPDAHLGCISYCA | 1146 |
| BCY16048 | none | ACPPDAHLGCISYCA | 1147 |
| BCY16963 | none | ACP[Aib]DAYLGC[tBuGly]SYCA | 1148 |
| BCY17987 | Ac | CSADAHLGCISYC | 1149 |
| BCY16753 | none | ACS[Aib]DAHLGC[tBuGly]SYCA | 1150 |
| BCY16046 | none | ACSPDAHLGC[EPA]SYCA | 1151 |
| BCY16964 | none | ACPPDAYLGC[tBuGly]SYCA | 1152 |
| BCY16965 | none | ACS[Aib]DAYLGC[tBuGly]SYCA | 1153 |
| BCY17986 | Ac | CAPDAHLGCISYC | 1154 |
| BCY16550 | none | ACP[Aib]DAHLGCISYCA | 1155 |
| BCY16966 | none | ACSPDAYLGC[tBuGly]SYCA | 1156 |
| BCY16051 | none | ACSPDAHLGC[tBuGly]SYCA | 1157 |
| BCY17118 | none | IDSNCPNDAHLGCISYCA | 1158 |
| BCY17116 | none | WGKSCPIDAHLGCISYCA | 1159 |
| BCY16053 | none | ACSPDAYLGCISYCA | 1160 |
| BCY16557 | none | ACPPDAYLGCISYCA | 1161 |
| BCY16035 | none | ACS[Aib]DAHLGCISYCA | 1162 |
| BCY16043 | none | ACSPDAHLGC[Chg]SYCA | 1163 |
| BCY15769 | none | ACAPDAHLGCISYCA[Sar]₆[K-F1] | 1164 |
| BCY15648 | none | ACYLPDW[tBuAla]CGDEYCA | 1165 |
| BCY16031 | none | ACSPDAHLGCIS[2Nal]CA | 1166 |
| BCY16079 | none | ACSPDAHLGCIS[₃tBuTyr]CA | 1167 |
| BCY16036 | none | ACSPD[Aib]HLGCISYCA | 1168 |
| BCY16029 | none | ACSPDAHLGCIS[1Nal]CA | 1169 |
| BCY16089 | none | ACSPDAH[tBuAla]GCISYCA | 1170 |
| BCY16088 | none | ACSPDAH[Cba]GCISYCA | 1171 |
| BCY16052 | none | ACSPDAHLGCISWCA | 1172 |
| BCY16033 | none | ACSPD[Abu]HLGCISYCA | 1173 |
| BCY16039 | none | ACS[Aze]DAHLGCISYCA | 1174 |
| BCY17988 | Ac | CS[Aze]DAHLGCISYC | 1175 |
| BCY17879 | Ac | CS[Aze]DAHLGCISYC[K(N₃)] | 1176 |
| BCY17880 | AzPro | CS[Aze]DAHLGCISYC | 1177 |
| BCY16038 | none | ACSPDDHLGCISYCA | 1178 |
| BCY16050 | none | ACSPDSHLGCISYCA | 1179 |
| BCY16034 | none | ACSPDAH[Abu]GCISYCA | 1180 |
| BCY16032 | none | ACSPDAHLGCIS[4Pal]CA | 1181 |
| BCY16049 | none | ACP[dA]DAHLGCISYCA | 1182 |
| BCY16558 | none | ACSPDAYLGC[tBuAla]SYCA | 1183 |
| BCY16041 | none | ACSPDAHLGC[C5g]SYCA | 1184 |
| BCY16042 | none | ACSPDAHLGC[Cbg]SYCA | 1185 |
| BCY16045 | none | ACSPDAHL[dA]CISYCA | 1186 |
| BCY16037 | none | ACSPDAH[Aib]GCISYCA | 1187 |
| BCY16044 | none | ACSPDAHLGC[Cpg]SYCA | 1188 |
| BCY16040 | none | ACSPDAHLGC[B-Melle]SYCA | 1189 |
| BCY15771 | none | ACSADAHLGCISYCA[Sar]₆[K-F1] | 1190 |
| BCY15772 | none | ACSPAAHLGCISYCA[Sar]₆[K-F1] | 1191 |
| BCY15773 | none | ACSPDAALGCISYCA[Sar]₆[K-F1] | 1192 |
| BCY15774 | none | ACSPDAHAGCISYCA[Sar]₆[K-F1] | 1193 |
| BCY15775 | none | ACSPDAHLACISYCA[Sar]₆[K-F1] | 1194 |
| BCY15776 | none | ACSPDAHLGCASYCA[Sar]₆[K-F1] | 1195 |
| BCY15777 | none | ACSPDAHLGCIAYCA[Sar]₆[K-F1] | 1196 |
| BCY15770 | none | ACSPDAHLGCISACA[Sar]₆[K-F1] | 1197 |
| BCY17903 | Ac | C[K(N₃)]PDAHLGCISYC | 1198 |
| BCY17904 | Ac | CS[K(N₃)]DAHLGCISYC | 1199 |
| BCY17905 | Ac | CSPD[K(N₃)]HLGCISYC | 1200 |
| BCY23180 | none | C[HyP][HyP]DAYLGC[tBuGly]SYCEPW | 1201 |
| BCY21757 | Ac | CP[HyP]DAYLGC[tBuGly]SYCEPWK | 1202 |
| BCY21758 | Ac | CP[HyP]DAYLGC[tBuGly]SYCEPWC | 1203 |
| BCY23181 | none | C[Oxa][HyP]DAYLGC[tBuGly]SYCEPW | 1204 |
| BCY23182 | none | C[Cis-HyP][HyP]DAYLGC[tBuGly]SYCEPW | 1205 |
| BCY23183 | none | CP[Oxa]DAYLGC[tBuGly]SYCEPW | 1206 |
| BCY23184 | none | CP[Cis-HyP]DAYLGC[tBuGly]SYCEPW | 1207 |
| BCY23185 | none | CP[HyP]DA[DOPA]LGC[tBuGly]SYCEPW | 1208 |
| BCY23186 | none | CP[HyP]DA[pCaPhe]LGC[tBuGly]SYCEPW | 1209 |
| BCY23187 | none | CP[HyP]DA[pCoPhe]LGC[tBuGly]SYCEPW | 1210 |
| BCY23188 | none | CP[HyP]DA[hTyr]LGC[tBuGly]SYCEPW | 1211 |
| BCY23189 | none | CP[HyP]DAYL[dS]C[tBuGly]SYCEPW | 1212 |
| BCY23190 | none | CP[HyP]DAYL[dT]C[tBuGly]SYCEPW | 1213 |
| BCY23191 | none | CP[HyP]DAYL[dD]C[tBuGly]SYCEPW | 1214 |
| BCY23192 | none | CP[HyP]DAYL[dE]C[tBuGly]SYCEPW | 1215 |
| BCY23193 | none | CP[HyP]DAYL[dN]C[tBuGly]SYCEPW | 1216 |
| BCY23194 | none | CP[HyP]DAYL[dQ]C[tBuGly]SYCEPW | 1217 |
| BCY23195 | none | CP[HyP]DAYL[dY]C[tBuGly]SYCEPW | 1218 |
| BCY23196 | none | CP[HyP]DAYLSC[tBuGly]SYCEPW | 1219 |
| BCY23197 | none | CP[HyP]DAYLDC[tBuGly]SYCEPW | 1220 |
| BCY23198 | none | CP[HyP]DAYLYC[tBuGly]SYCEPW | 1221 |
| BCY23199 | none | CP[HyP]DAYLNC[tBuGly]SYCEPW | 1222 |
| BCY23200 | none | CP[HyP]DAYLGC[tBuGly]S[DOPA]CEPW | 1223 |
| BCY23201 | none | CP[HyP]DAYLGC[tBuGly]S[pCaPhe]CEPW | 1224 |
| BCY23202 | none | CP[HyP]DAYLGC[tBuGly]S[pCoPhe]CEPW | 1225 |
| BCY23203 | none | CP[HyP]DAYLGC[tBuGly]S[hTyr]CEPW | 1226 |
| BCY23204 | none | CP[HyP]DAYLGC[tBuGly]SYCE[HyP]W | 1227 |
| BCY23205 | none | CP[HyP]DAYLGC[tBuGly]SYCE[Oxa]W | 1228 |
| BCY23206 | none | CP[HyP]DAYLGC[tBuGly]SYCE[Cis-HyP]W | 1229 |
| BCY23207 | none | CP[HyP]DAYLGC[tBuGly]SYCEPY | 1230 |
| BCY23208 | none | CP[HyP]DAYLGC[tBuGly]SYCEP[DOPA] | 1231 |
| BCY23209 | none | CP[HyP]DAYLGC[tBuGly]SYCEP[pCaPhe] | 1232 |
| BCY23210 | none | CP[HyP]DAYLGC[tBuGly]SYCEP[pCoPhe] | 1233 |
| BCY23211 | none | CP[HyP]DAYLGC[tBuGly]SYCEP[hTyr] | 1234 |
| BCY23216 | none | CP[HyP]EAYLGC[tBuGly]SYCEPW | 1235 |
| BCY23217 | none | CP[HyP][Gla]AYLGC[tBuGly]SYCEPW | 1236 |
| BCY23218 | none | CP[HyP]DAYSGC[tBuGly]SYCEPW | 1237 |
| BCY23219 | none | CP[HyP]DAYTGC[tBuGly]SYCEPW | 1238 |
| BCY23220 | none | CP[HyP]DAYDGC[tBuGly]SYCEPW | 1239 |
| BCY23221 | none | CP[HyP]DAYEGC[tBuGly]SYCEPW | 1240 |
| BCY23222 | none | CP[HyP]DAYNGC[tBuGly]SYCEPW | 1241 |
| BCY23223 | none | CP[HyP]DAYQGC[tBuGly]SYCEPW | 1242 |
| BCY23224 | none | CP[HyP]DAYLGC[tBuGly][HSer]YCEPW | 1243 |
| BCY23225 | none | CP[HyP]DAYLGC[tBuGly]TYCEPW | 1244 |
| BCY23226 | none | CP[HyP]DAYLGC[tBuGly]DYCEPW | 1245 |
| BCY23227 | none | CP[HyP]DAYLGC[tBuGly]EYCEPW | 1246 |
| BCY23228 | none | CP[HyP]DAYLGC[tBuGly]NYCEPW | 1247 |
| BCY23229 | none | CP[HyP]DAYLGC[tBuGly]QYCEPW | 1248 |
| BCY23230 | none | CP[HyP]DAYLGC[tBuGly]SYCDPW | 1249 |
| BCY23231 | none | CP[HyP]DAYLGC[tBuGly]SYC[Gla]PW | 1250 |
| BCY23514 | none | CP[HyP]DAYLGCYSYCEPW | 1251 |
| BCY23515 | none | CP[HyP]DAYLGC[3HyV]SYCEPW | 1252 |

wherein Ac represents acetyl, AzPro represents azidopropyl, Au represents amino butyric acid, Aib represents aminoisobutyric acid, Aze represents azetidine, B-Melle represents beta-methyl isoleucine, C5g represents cyclopentyl glycine, Cba represents 0˜-cyclobutylalanine, Cbg represents cyclobutyl glycine, Chg represents cyclohexyl glycine, Cpg represents cyclopropyl glycine, EPA represents 2-amino-3-ethyl-pentanoic acid, HyP represents trans-4-hydroxy-L-proline, K(N₃) represents 6-azido lysine, 1Nal represents 1-naphthylalanine, 2Nal represents 2-naphthylalanine, 4Pal represents 4-pyridylalanine, Pip represents pipecolic acid; tBuAla represents t-butyl-alanine, tBuGly represents t-butyl-glycine, 3tBuTyr represents 3-t-Butyl-Tyrosine, Sar represents sarcosine, K-Fl represents fluorescein attached at the 6-position of a lysine, Fl-G represents fluorescein attached at the N-terminus of a glycine, NMeTrp represents N-methyl tryptophan, dP represents _{D}-proline, dA represents _{D}-alanine, dW represents _{D}-tryptophan, dS represents _{D}-serine, dT represents _{D}-threonine, dD represents _{D}-aspartic acid, dE represents _{D}-glutamic acid, dN represents _{D}-asparagine, dQ represents _{D}-glutamine, dY represents _{D}-tyrosine, Cis-HyP represents cis-L-4-hydroxyproline, DOPA represents 3,4-dihydroxy-phenylalanine, Gla represents L-γ-carboxyglutamic acid, HSer represents homoserine, hTyr represents homo-tyrosine, 3HyV represents 3-hydroxy-L-valine, Oxa represents oxazolidine-4-carboxylic acid, pCaPhe represents L-4-carbamoylphenylalanine, pCoPhe represents 4-carboxy-L-phenylalanine, and [K(N₃)(PYA-Maleimide)] represents a modified lysine having the following structure:

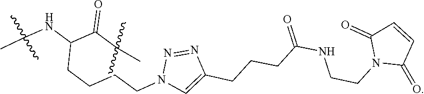 [SMILES: *NC(CCCCn1cc(CCCC(=O)NCCN2C(=O)C=CC2=O)nn1)C(*)=O]

In certain embodiments, a bicycle ligand, or a conjugate group comprising a bicycle ligand, has the structure:

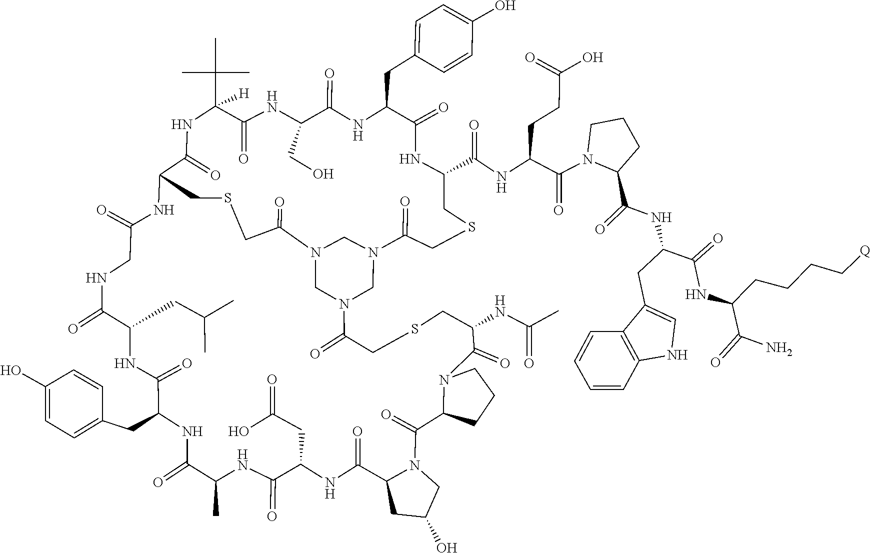

or a salt thereof, wherein Q is N₃ (BCY17901, SEQ ID NO: 1045), NH₂ (BCY21757, SEQ ID NO: 1202), SH (BCY21758, SEQ ID NO: 1203), a conjugate linker, or a conjugate linker covalently connected to an oligonucleotide.

In certain embodiments, a bicycle ligand comprises an amino acid sequence which is selected from CP[HyP]DAYLGC[tBuGly]SYCEPWK (SEQ ID NO: 1202, herein referred to as BCY21757) and CP[HyP]DAYLGC[tBuGly]SYCEPWC (SEQ ID NO: 1203, herein referred to as BCY21758), wherein HyP represents trans-4-hydroxy-L-proline and tBuGly represents t-butyl-glycine. In certain embodiments, the bicycle ligand comprises an N-terminal acetyl group and a C-terminal CONH₂ group. In certain embodiments, the first, second, and third cysteine residues within the bicycle ligand are covalently bonded to a molecular scaffold such that two peptide loops are formed on the molecular scaffold. In certain embodiments, the molecular scaffold is 1,1′,1″-(1,3,5-triazinane-1,3,5-triyl)triprop-2-en-1-one (TATA). In certain embodiments, the molecular scaffold is 1,1′,1″-(1,3,5-triazinane-1,3,5-triyl)tris(2-bromoethanone) (TATB).

### Transferrin Receptor

In certain embodiments, a bicycle ligand is capable of interacting with the type 1 transferrin receptor (e.g., a human type 1 transferrin receptor). In certain embodiments, a bicycle ligand is capable of binding the type 1 transferrin receptor. In certain embodiments, a bicycle ligand is capable of binding the type 1 transferrin receptor while not interfering with the binding of the natural ligand transferrin. In certain embodiments, a bicycle ligand inhibits the binding of the natural ligand transferrin.

### Alternative Transferrin Receptor Ligands

Alternative cell-targeting moieties that have affinity for transferrin receptor (TfR1), including antibodies and antibody fragments, proteins, peptides, and aptamers, have been described. Such moieties may replace a bicycle ligand conjugate moiety described herein for targeting an oligomeric compound to cardiac cells (e.g., cardiac muscle cells)/tissue/heart. Thus, in certain embodiments, a conjugate group comprises a cell-targeting moiety that binds transferrin receptor (TfR). In certain embodiments, a conjugate group described herein comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, the anti-TfR1 antibody or fragment thereof is any known in the art including but not limited to those described in WO1991/004753; WO2013/103800; WO2014/144060; WO2016/081643; WO2016/179257; WO2016/207240; WO2017/221883; WO2018/129384; WO2018/124121; WO2019/151539; WO2020/132584; WO2020/028864; U.S. Pat. Nos. 7,208,174; 9,034,329; 10,550,188; and 11,512,136. In certain embodiments, a fragment of an anti-TfR1 antibody is F(ab′)₂, Fab, Fab′, Fv, scFv, VHH, or VNAR. In certain embodiments, an antibody binds to TfR1 through an engineered Fc domain rather than through the antigen-binding portion, as described in, e.g., US 2020/0223935. In certain embodiments, the conjugate group comprises a protein or peptide capable of binding TfR1. In certain embodiments, the protein or peptide capable of binding TfR1 is any known in the art including but not limited to those described in WO2019/140050; WO2020/037150; WO2020/124032; WO 2022/026555; and U.S. Pat. No. 10,138,483. In certain embodiments, the peptide is a cyclic peptide, as described in WO 2021/167107. In certain embodiments, the conjugate group comprises an aptamer capable of binding TfR1. In certain embodiments, the aptamer capable of binding TfR1 is any known in the art including but not limited to those described in WO2013/163303; WO2019/033051; and WO2020/245198. In certain embodiments, the conjugate group comprises a peptide, including but not limited to a cyclic peptide, capable of binding TfR1. In certain embodiments, the peptide capable of binding TfR1 is any known in the art including but not limited to those described in EP4108676; WO2023/027125; and WO2023/022234.

### Conjugate Linkers

In certain embodiments, oligomeric compounds comprise an oligonucleotide and a conjugate group, wherein the conjugate group comprises or consists of a conjugate moiety and a conjugate linker. In certain embodiments, the conjugate linker links the conjugate moiety to the oligonucleotide. In certain embodiments, the conjugate linker is a single chemical bond (i.e., the conjugate moiety is attached directly to an oligonucleotide through a single bond). In certain embodiments, the conjugate linker comprises one or more atoms. In certain embodiments, the conjugate linker comprises a chemical group. In certain embodiments, the conjugate linker comprises a chain structure, such as a hydrocarbyl chain, or an oligomer of repeating units such as ethylene glycol, nucleosides, or amino acid units. In certain embodiments, the oligonucleotide is a modified oligonucleotide. In certain embodiments, the conjugate moiety is a bicycle ligand. In certain embodiments, the conjugate moiety comprises two peptide loops attached to a molecular scaffold.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises one or more groups selected from alkyl, amino, oxo, amide and ether groups. In certain embodiments, the conjugate linker comprises one or more groups selected from alkyl and amide groups. In certain embodiments, the conjugate linker comprises one or more groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described herein, are bifunctional linking moieties, e.g., those known in the art to be useful for attaching conjugate moieties to parent compounds, such as the oligonucleotides provided herein. In general, a bifunctional linking moiety comprises at least two functional groups. One of the functional groups is selected to react with a particular site on a parent compound and the other is selected to react with a conjugate moiety. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

In certain embodiments, conjugate linkers comprise chemical groups that are formed upon a reaction between a first functional group and a second functional group. In certain embodiments, a modified oligonucleotide is attached to the first functional group during synthesis, and a conjugate moiety is attached to a second functional group during synthesis. Then, the two compounds are mixed under specific conditions to yield the oligomeric compound. In certain embodiments, the conjugate moiety is a bicycle ligand. In certain embodiments, the conjugate moiety comprises two peptide loops attached to a molecular scaffold. Such reactions that are compatible with both oligonucleotide and peptide chemistry have been previously described and are often called “bioconjugation” reactions. These reactions include strain promoted azido-alkyne cycloaddition (SPAAC), copper-catalyzed click reaction (CuAAC), active ester conjugation to an amino modified oligonucleotide, maleimide-thiol Michael addition, ketol/hydroxylamine ligation, the Staudinger ligation, reductive ainiation, thio ether formation, disulfide formation, reductive alkylation, catalyst-free N-arylation, sulfur fluoride exchange click reaction (SuFEx), and inverse demand Diels Alder reaction. Certain such reactions are described in, e.g., Jbara, et al., “Oligonucleotide Bioconjugation with Bifunctional Palladium Reagents”, Angew. Chem. Int. Ed. 2021, 60(21)12109-12115; Dong, et al., “Sulfur(VI) Fluoride Exchange (SuFEx): Another Good Reaction for Click Chemistry,” Angew. Chem. Int. Ed. 2014, 53(36):9430-9448.4; Zhang, et al., “Arylation Chemistry for Bioconjugation,” Angew. Chem. Int. Ed. Engl. 2019; 58(15): 4810-4839; Walsh, et al., “Site-selective modification strategies in antibody-drug conjugates” Chem. Soc. Rev., 2021, 50: 1305-1353; Tiefenbrunn, et al., “Chemoselective ligation techniques: modem applications of time-honored chemistry”, Biopolymers, 2010, 94(1):95-106; Drake, et al., Bioconjug. Chem. 2014, 25(7):1331-1341; Bode, Acc. Chem. Res., 2017, 50, 9, 2104-2115; J. Magano, B. Bock, et al, Org. Proc. Res. Dev. 2014, 18:142-151; Craig S. McKay and M. G. Finn, Chem. Biol. 2014, 21 (9), 1075-1101; Mitchell P. Christy et al., Org. Lett. 2020, 22: 2365; Ren et al., Angew. Chem. Int. Ed. Engl. 2009, 48, 9658-9662; Rohrbacher, F. et al., Helv. Chim. Acta. 2018, 101 (5), e1800039; Baalmaan, et al, “A Bioorthogonal Click Chemistry Toolbox for Targeted Synthesis of Branched and Well-Defined Protein-Protein Conjugates”, Angew. Chem. Int. Ed. 2020 (59): 12885-12893; Lang, et al, “Biorthogonal Reactions for Labeling Proteins”, J. Am. Chem. Soc, 2014, 9(1):16-20; Nair, et al., “The Thiol-Michael Addition Click Reaction: A Powerful and Widely Used Tool in Materials Chemistry”, Chem. Mater. 2013 26(1):724-744; Kalia and Raines, “Hydrolytic Stability of Hydrazones and Oximes”, Angew. Chem. Int. Ed., 2008, 47:7523-7526.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, the bicycle ligand comprises an N-terminal or a C-terminal extension azide group, which optionally may be joined with an oligonucleotide or oligomeric compound by cycloaddition with bicyclo[6.1.0]non-4-yn-9-ylmethyl carbamate-oligo or 2-(cyclooct-2-yn-1-yloxy)acetamide-oligo. In certain embodiments, the bicycle ligand comprises an N-terminal or a C-terminal extension amide group which optionally may be joined with an oligonucleotide or oligomeric compound by coupling with oligo-7-amido-7-oxoheptanoic acid. In certain embodiments, the bicycle ligand comprises an N-terminal or a C-terminal extension 2-(aminooxy)acetamide group which optionally may be joined with an oligonucleotide or oligomeric compound by condensation with 5-oxo-5-(4-oxopiperidin-1-yl)pentanamide-oligo. In certain embodiments, the bicycle ligand comprises an N-terminal or a C-terminal extension thiol group which optionally may be joined with an oligonucleotide or oligomeric compound by addition to 3-(2,5-dioxo-2,5-dihydro-1H-pyrrol-1-yl)propenamide-oligo.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a sugar surrogate. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methyl cytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) an oligonucleotide consisting of 8-30 nucleosides and (2) a conjugate linker comprising 1-10 linker-nucleosides that are contiguous with the nucleosides of the oligonucleotide. The total number of contiguous linked nucleosides in such an oligomeric compound is more than 30. Alternatively, an oligomeric compound may comprise an oligonucleotide consisting of 8-30 nucleosides and no conjugate linker. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate moiety to be cleaved from the oligonucleotide. For example, in certain circumstances oligomeric compounds comprising a particular conjugate moiety are better taken up by a particular cell type, but once the oligomeric compound has been taken up, it is desirable that the conjugate moiety be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphodiester linkage between an oligonucleotide and a conjugate moiety.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphodiester internucleoside linkage and covalently attached to the remainder of the conjugate linker or conjugate moiety by a phosphodiester or phosphorothioate linkage. In certain such embodiments, the cleavable moiety comprises or is a 2′-deoxyadenosine.

In certain embodiments, oligomeric compounds described herein comprise an oligonucleotide linked to a conjugate moiety by a conjugate linker, wherein the oligomeric compound is prepared using Click chemistry known in the art. Compounds have been prepared using Click chemistry wherein alkynyl phosphonate internucleoside linkages on an oligomeric compound attached to a solid support are converted into the 1,2,3-triazolylphosphonate internucleoside linkages and then cleaved from the solid support (Krishna et al., J. Am. Chem. Soc. 2012, 134(28), 11618-11631), which is incorporated by reference herein in its entirety. Additional conjugate linkers suitable for use in several embodiments are prepared by Click chemistry described in “Click Chemistry for Biotechnology and Materials Science” Ed. Joerg Laham, Wiley 2009, which is incorporated by reference herein in its entirety. Further examples of linking chemistry include an inverse electron demand Diels-Alder reaction, e.g., as described in Argamunt et al., J. Org. Chem. 2020, 85, 10, 6593-6604, Sarrett et al., Nat. Protocols 2021, 16, 3348-3381; Handula et al., Molecules, 2021, 26 (15), 4640, Wiessler et al., Int. J. Med. Sci. 2010, 7 (1), 19-28; copper-catalyzed azide-alkyne cycloaddition (CuAAC) see, e.g., S. I. Presolski, et al., J. Am. Chem. Soc. 2010, 132, 14570-14576; D. Soriano Del Amo, et al., J. Am. Chem. Soc., 2010, 132, 16893-16899; Staudinger reaction, see, e.g., Saxon and C. R. Bertozzi, Science, 2000, 287, 2007-2010; B. L. Nilsson, et al., Org. Lett., 2000, 2, 1939-1941, E. Saxon, et al., Org. Lett., 2000, 2, 2141-2143; formation of hydrazones and oximes, see, e.g., J. Y. Axup, et al., Proc. Natl. Acad. Sci. U.S.A, 2012, 109, 16101-16106; photoclick reactions, see, e.g., W. Song, et al., Angew. Chem., Int. Ed., 2008, 47, 2832-2835, A. Herner and Q. Lin, Top. Curr. Chem., 2016, 374, 1; strain-promoted alkyne-nitrone cycloaddition (SPANC) reactions, see, e.g., D. A. MacKenzie, et al., Curr. Opin. Chem. Biol., 2014, 21, 81-88; transition metal catalyzed cross coupling, see, e.g., M. Chalker, et al., J. Am. Chem. Soc., 2009, 131, 16346-16347; nucleophilic additions, in particular, of a thiol to a maleimide, see, e.g., Kang et al., Chem. Sci., 2021, 12, 13613-13647, Bernardim et al., Nat. Comm. 2016, 7, 13128, Jain et al., Pharm. Res. 2015, 32 (11), 3526-3540, each of which is incorporated by reference herein in its entirety.

In certain embodiments, the conjugate linker is prepared by reaction of a first reactive moiety with a second reactive moiety, wherein the first reactive moiety is attached to the oligonucleotide and the second reactive moiety is attached to the conjugate moiety, or a precursor thereof. In certain embodiments, the conjugate linker is prepared by reaction of a dipolarophile (e.g., a triple bonded moiety such as an alkyne or nitrile) with a 1,3-dipole (e.g., an azide, a nitrone, an isocyanate, or a thioisocyanate):

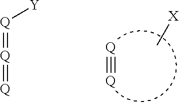

wherein each Q is independently a carbon atom or a heteroatom, one of X and Y is attached to an oligonucleotide, and the other of X and Y is attached to a conjugate moiety. The conjugate linker thus prepared may comprise a five-membered unsaturated heterocyclic ring such as a triazole.

In certain embodiments, the conjugate linker is prepared by reaction of a dieneophile (e.g., an electron rich double bond such as a furan or derivative thereof) with an electron poor diene (e.g., a tetrazine):

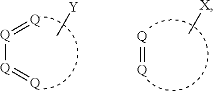

wherein each Q is independently a carbon atom or a heteroatom, one of X and Y is attached to an oligonucleotide, and the other of X and Y is attached to a conjugate moiety. The conjugate linker thus prepared may comprise a six-membered unsaturated heterocyclic ring such as a dihydropyrazine.

In certain embodiments, the conjugate linker is prepared by reaction of a nucleophile (e.g., a thiol or amine) with an electrophile (e.g., an electron-poor carbonyl or carbonyl-conjugated alkene or alkyne):

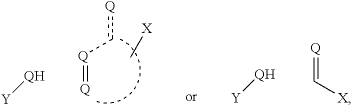

wherein each Q is independently a carbon atom or a heteroatom, one of X and Y is attached to an oligonucleotide, and the other of X and Y is attached to a conjugate moiety. The conjugate linker thus prepared may comprise a thioether, hydrazone, oxime, or amide.

Each of the first reactive moiety and the second reactive moiety may attach at any suitable position of the oligonucleotide and the conjugate moiety, for example, at a position described herein. In certain embodiments, the conjugate moiety comprises a peptide or polypeptide. In certain embodiments, the second reactive moiety is attached to an amino-acid side chain of a peptide or polypeptide. In certain embodiments, the second reactive moiety is attached to the N-terminus of the peptide or polypeptide. In certain embodiments, the second reactive moiety is attached to the C-terminus of a peptide or polypeptide. In certain embodiments, the second reactive moiety replaces the amino group of a lysine of a peptide or polypeptide.

In certain embodiments, a Click reaction is used to link a conjugate moiety and an oligonucleotide by reacting:

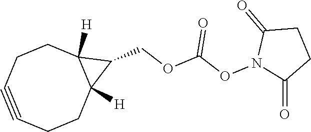 [SMILES: O=C(OC[C@@H]1[C@@H]2CCC#CCC[C@@H]21)ON1C(=O)CCC1=O]

with an oligonucleotide having a terminal amine, including but not limited to the following compound:

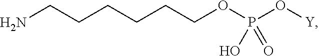

wherein Y is the oligonucleotide, to yield:

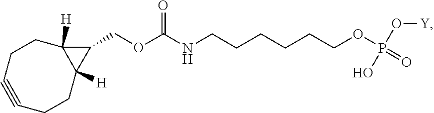

which is reacted with a conjugate moiety having an azide to yield:

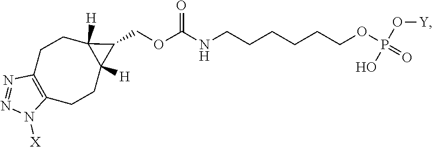

wherein N—N═N is formed from an azido group of the conjugate moiety, and wherein X represents the remainder of the conjugate moiety. In certain embodiments, the conjugate moiety comprises a bicycle ligand. In certain embodiments, the conjugate moiety comprises a peptide or polypeptide. In certain embodiments, the azido group is attached to an amino-acid side chain of the peptide or polypeptide. In certain embodiments, the azido group is attached to the N-terminus of the peptide or polypeptide. In certain embodiments, the azido group is attached to the C-terminus of the peptide or polypeptide. In certain embodiments, the azido group replaces the amino group of a lysine of the peptide or polypeptide.

In certain embodiments, an oligomeric compound comprises an oligonucleotide linked to a conjugate moiety by a conjugate linker, wherein the conjugate linker is prepared from the following compound:

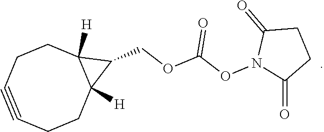 [SMILES: O=C(OC[C@@H]1[C@@H]2CCC#CCC[C@@H]21)ON1C(=O)CCC1=O]

In certain embodiments, an oligomeric compound comprises an oligonucleotide linked to a conjugate moiety by a conjugate linker, wherein the conjugate linker comprises:

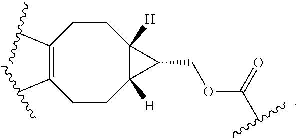 [SMILES: *C(=O)OC[C@@H]1[C@@H]2CC/C(*)=C(/*)CC[C@@H]21]

In certain embodiments, an oligomeric compound comprises an oligonucleotide linked to a conjugate moiety by a conjugate linker, wherein the conjugate linker comprises:

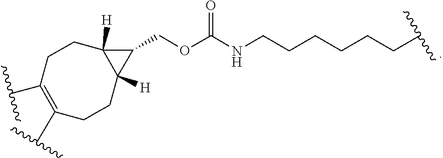 [SMILES: *CCCCCCNC(=O)OC[C@@H]1[C@@H]2CC/C(*)=C(/*)CC[C@@H]21]

In certain embodiments, an oligomeric compound comprises an oligonucleotide linked to a conjugate moiety by a conjugate linker, wherein the compound comprises:

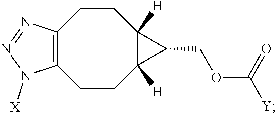

Wherein N—N═N is formed from an azido group of the conjugate moiety; X represents the remainder of the conjugate moiety; and Y represents a portion of the oligomeric compound comprising the oligonucleotide. In certain embodiments, the conjugate moiety comprises a bicycle ligand. In certain embodiments, the conjugate moiety comprises a peptide or polypeptide. In certain embodiments, the azido group is attached to an amino-acid side chain of the peptide or polypeptide. In certain embodiments, the azido group is attached to the N-terminus of the peptide or polypeptide. In certain embodiments, the azido group is attached to the C-terminus of the peptide or polypeptide. In certain embodiments, the azido group replaces the amino group of a lysine of the peptide or polypeptide.

In certain embodiments, an oligomeric compound comprises an oligonucleotide linked to a conjugate moiety by a conjugate linker, wherein the oligomeric compound comprises:

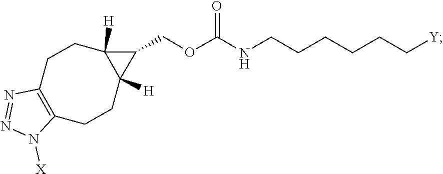

wherein N—N═N is formed from an azido group of the conjugate moiety; X represents the remainder of the conjugate moiety; and Y represents the oligonucleotide. In certain embodiments, the conjugate moiety comprises a bicycle ligand. In certain embodiments, the conjugate moiety comprises a peptide or polypeptide. In certain embodiments, the azido group is attached to an amino-acid side chain of the peptide or polypeptide. In certain embodiments, the azido group is attached to the N-terminus of the peptide or peptide or polypeptide. In certain embodiments, the azido group is attached to the C-terminus of the peptide or polypeptide. In certain embodiments, the azido group replaces the amino group of a lysine of the peptide or polypeptide.

In certain embodiments, an oligomeric compound comprises an oligonucleotide linked to a conjugate moiety by a conjugate linker, wherein the oligomeric compound comprises:

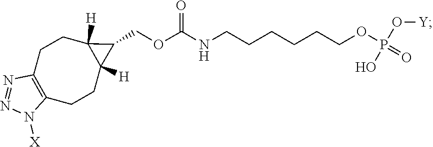

wherein N—N═N is formed from an azido group of the conjugate moiety; X represents the remainder of the conjugate moiety; and Y represents the oligonucleotide. In certain embodiments, the conjugate moiety comprises a bicycle ligand. In certain embodiments, the conjugate moiety comprises a peptide or polypeptide. In certain embodiments, the azido group is attached to an amino-acid side chain of the peptide or polypeptide. In certain embodiments, the azido group is attached to the N-terminus of the peptide or polypeptide. In certain embodiments, the azido group is attached to the C-terminus of the peptide or polypeptide. In certain embodiments, the azido group replaces the amino group of a lysine of the peptide or polypeptide.

In certain embodiments, a Click reaction is used to link a conjugate moiety and an oligonucleotide by reacting:

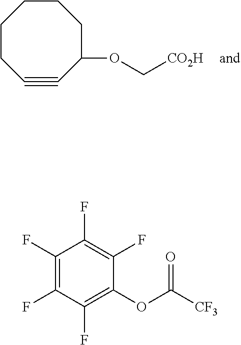 [SMILES: O=C(O)COC1C#CCCCCC1 ; O=C(Oc1c(F)c(F)c(F)c(F)c1F)C(F)(F)F]

in solution together
with an oligonucleotide having a terminal amine, including but not limited to the following compound:

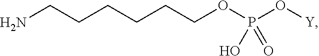

wherein Y is the oligonucleotide, to yield:

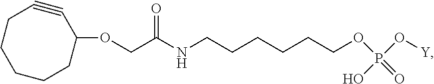

which is reacted with a conjugate moiety having an azide to yield:

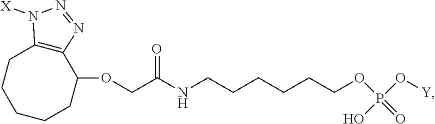

wherein N—N═N is formed from an azido group of the conjugate moiety, and wherein X represents the remainder of the conjugate moiety. In certain embodiments, the conjugate moiety comprises a bicycle ligand. In certain embodiments, the conjugate moiety comprises a peptide or polypeptide. In certain embodiments, the azido group is attached to an amino-acid side chain of the peptide or polypeptide. In certain embodiments, the azido group is attached to the N-terminus of the peptide or polypeptide. In certain embodiments, the azido group is attached to the C-terminus of the peptide or polypeptide. In certain embodiments, the azido group replaces the amino group of a lysine of the peptide or polypeptide.

In certain embodiments, an oligomeric compound comprises an oligonucleotide linked to a conjugate moiety by a conjugate linker, wherein the conjugate linker is prepared from the following compound:

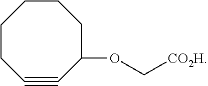 [SMILES: O=C(O)COC1C#CCCCCC1]

In certain embodiments, an oligomeric compound comprises an oligonucleotide linked to a conjugate moiety by a conjugate linker, wherein the conjugate linker comprises:

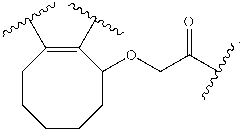 [SMILES: *C(=O)COC1CCCCC/C(*)=C\1*]

In certain embodiments, an oligomeric compound comprises an oligonucleotide linked to a conjugate moiety by a conjugate linker, wherein the conjugate linker comprises:

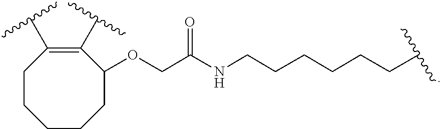 [SMILES: *CCCCCCNC(=O)COC1CCCCC/C(*)=C\1*]

In certain embodiments, an oligomeric compound comprises an oligonucleotide linked to a conjugate moiety by a conjugate linker, wherein the compound comprises:

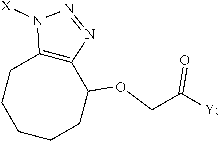

wherein N—N═N is formed from an azido group of the conjugate moiety; X represents the remainder of the conjugate moiety; and Y represents a portion of the oligomeric compound comprising the oligonucleotide. In certain embodiments, the conjugate moiety comprises a bicycle ligand. In certain embodiments, the conjugate moiety comprises a peptide or polypeptide. In certain embodiments, the azido group is attached to an amino-acid side chain of the peptide or polypeptide. In certain embodiments, the azido group is attached to the N-terminus of the peptide or polypeptide. In certain embodiments, the azido group is attached to the C-terminus of the peptide or polypeptide. In certain embodiments, the azido group replaces the amino group of a lysine of the peptide or polypeptide.

In certain embodiments, an oligomeric compound comprises an oligonucleotide linked to a conjugate moiety by a conjugate linker, wherein the oligomeric compound comprises:

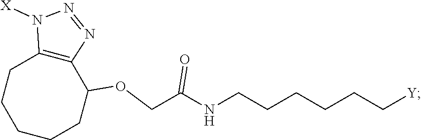

wherein N—N═N is formed from an azido group of the conjugate moiety; X represents the remainder of the conjugate moiety; and Y represents the oligonucleotide. In certain embodiments, the conjugate moiety comprises a bicycle ligand. In certain embodiments, the conjugate moiety comprises a peptide or polypeptide. In certain embodiments, the azido group is attached to an amino-acid side chain of the peptide or polypeptide. In certain embodiments, the azido group is attached to the N-terminus of the peptide or polypeptide. In certain embodiments, the azido group is attached to the C-terminus of the peptide or polypeptide. In certain embodiments, the azido group replaces the amino group of a lysine of the peptide or polypeptide.

In certain embodiments, an oligomeric compound comprises an oligonucleotide linked to a conjugate moiety by a conjugate linker, wherein the oligomeric compound comprises:

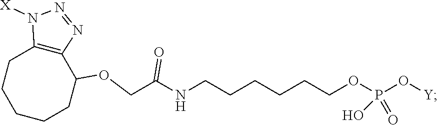

wherein N—N═N is formed from an azido group of the conjugate moiety; X represents the remainder of the conjugate moiety; and Y represents the oligonucleotide. In certain embodiments, the conjugate moiety comprises a bicycle ligand. In certain embodiments, the conjugate moiety comprises a peptide or polypeptide. In certain embodiments, the azido group is attached to an amino-acid side chain of the peptide or polypeptide. In certain embodiments, the azido group is attached to the N-terminus of the peptide or polypeptide. In certain embodiments, the azido group is attached to the C-terminus of the peptide or polypeptide. In certain embodiments, the azido group replaces the amino group of a lysine of the peptide or polypeptide.

In certain embodiments, an oligomeric compound comprises an oligonucleotide linked to a conjugate moiety by a conjugate linker, wherein the oligomeric compound comprises:

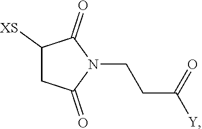

wherein X comprises the conjugate moiety; and Y comprises the oligonucleotide.

In certain embodiments, an oligomeric compound comprises an oligonucleotide linked to a conjugate moiety by a conjugate linker, wherein the oligomeric compound comprises:

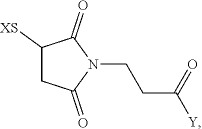

wherein X comprises the oligonucleotide; and Y comprises the conjugate moiety.

In certain embodiments, an oligomeric compound comprises an oligonucleotide linked to a conjugate moiety by a conjugate linker, wherein the conjugate linker comprises:

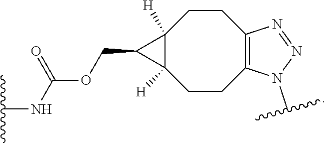 [SMILES: *NC(=O)OC[C@@H]1[C@@H]2CCc3c(nnn3*)CC[C@@H]21]

In certain embodiments, an oligomeric compound comprises an oligonucleotide linked to a conjugate moiety by a conjugate linker, wherein the conjugate linker comprises:

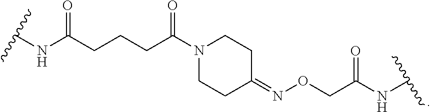 [SMILES: *NC(=O)CCCC(=O)N1CCC(=NOCC(=O)N*)CC1]

In certain embodiments, an oligomeric compound comprises an oligonucleotide linked to a conjugate moiety by a conjugate linker, wherein the conjugate linker comprises:

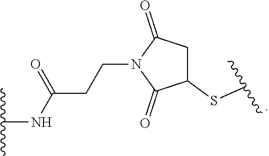 [SMILES: *NC(=O)CCN1C(=O)CC(S*)C1=O]

In certain embodiments, an oligomeric compound comprises an oligonucleotide linked to a conjugate moiety by a conjugate linker, wherein the conjugate linker comprises:

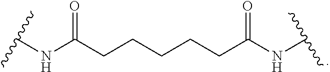 [SMILES: *NC(=O)CCCCCC(=O)N*]

In certain embodiments, an oligomeric compound comprises an oligonucleotide linked to a conjugate moiety by a conjugate linker, wherein the conjugate linker comprises:

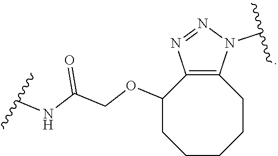 [SMILES: *NC(=O)COC1CCCCCc2c1nnn2*]

In certain embodiments, a Click reaction is used to link a conjugate moiety and an oligonucleotide by reacting:

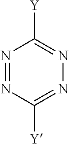

with

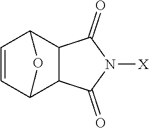

wherein one of Y and Y′ is attached to the conjugate moiety and X is attached to the oligonucleotide, to yield:

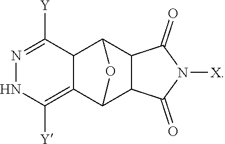

Synthetic methods describing preparation of the above starting materials and intermediates can be found in one or more of the following: Agard, N. J., et al. “A Strain-Promoted [3+2] Azide-Alkyne Cycloaddition for Covalent Modification of Biomolecules in Living Systems.” J. Am. Chem. Soc. 2004, 126, 15046-15047; Lang, K., and Chin, J. W. “Biorthogonal Reactions for Labeling Proteins.” ACS Chem. Biol. 2014, 9 (1), 16-20; Nair, D. P. et al. “The Thiol-Michael Addition Click Reaction: A Powerful and Widely Used Tool in Materials Chemistry.” Chem. Mater. 2013 26 (1), 724-744; WO2011/136645; K61mel, D. K. and Kool, E. T. “Oximes and Hydrazones in Bioconjugation: Mechanism and Catalysis.” Chem. Rev. 2017, 117, 10358-10376; Wang, J. et al. “Polyfluorophenyl Ester-Terminated Homobifunctional Cross-Linkers for Protein Conjugation.” Synlett, 2017, 28 (15), 1934-1938; Kishimoto, S. et al. “Site-Specific Chemical Conjugation of Antibodies by Using Affinity Peptide for the Development of Therapeutic Antibody Format,” Bioconjugate Chem., 2019, 30 (3), 698-702, Wu and Devaraj, “Inverse Electron-Demand Diels-Alder Bioorthogonal Reactions,” Top. Curr. Chem. 2016, 374, 3, Oliveira et al., “Inverse electron demand Diels-Alder reactions in chemical biology,” Chem. Soc. Rev., 2017, 46, 4895-4950.

Synthetic methods describing preparation of conjugated bicycle peptide ligands can be found in one or more of the following: International Patent Application Publication No. WO2022/101633, International Patent Application Publication No. WO2023/056388, each of which is hereby incorporated by reference. Bicycle peptides are synthesized by standard techniques, followed by reaction with a molecular scaffold using standard chemistry to form bicycle compound. See also, e.g., Timmerman et al., Chembiochem, 2005, 6: 821-824; Heinis et al., Angewandte Chemie, International Edition 2014, 53: 1602-1606; van de Langemheen et al., Chembiochem, 2017, 18: 387-395. Standard bio-conjugation, chemistry, and/or coupling techniques may be used to introduce, e.g., activated or activatable N- and/or C-terminal groups, or through side chains, including utilizing linkers described herein, to introduce additional components to conjugate compounds herein.

### B. Certain Terminal Groups

As used herein, “terminal group” means a chemical group or group of atoms that is covalently linked to a terminus of an oligonucleotide. Examples of a terminal group include, but are not limited to, a capping group, a phosphate moiety, a stabilized phosphate group, a protecting group, a modified or unmodified nucleoside, and two or more nucleosides that are independently modified or unmodified, wherein one or more groups is attached to either or both ends of an oligonucleotide. In certain embodiments, one or more terminal groups is attached to either or both ends of an oligonucleotide. In certain embodiments, one or more terminal groups is attached at the 3′ and/or 5′-end of the oligonucleotide. In certain embodiments, one or more terminal groups is attached at the 3′-end of the oligonucleotide. In certain embodiments, one or more terminal groups is attached at the 5′-end of the oligonucleotide. In certain embodiments, one or more terminal groups is attached at the 3′-end of the oligonucleotide and one or more terminal groups is attached at the 5′-end of the oligonucleotide. In certain embodiments, a terminal group is attached at the 3′ and/or 5′-end of the oligonucleotide. In certain embodiments, a terminal group is attached at the 3′-end of the oligonucleotide. In certain embodiments, a terminal group is attached near the 3′-end of the oligonucleotide. In certain embodiments, a terminal group is attached at the 5′-end of the oligonucleotide. In certain embodiments, a terminal group is attached near the 5′-end of the oligonucleotide. In certain embodiments, a terminal group is attached at the 3′-end of the oligonucleotide and a terminal group is attached at the 5′-end of the oligonucleotide.

In certain embodiments, an oligomeric compound comprises one or more terminal groups. In certain embodiments, an oligomeric compound comprises a terminal group comprising a stabilized 5′-phosphate. In certain embodiments, a stabilized phosphate group results in stabilization of a 5′-phosphate moiety of the 5′-terminal nucleoside of an oligonucleotide, relative to the stability of an unmodified 5′-phosphate of a nucleoside under biologic conditions. Such stabilization of a 5′-phosphate group includes but is not limited to resistance to removal by phosphatases. Stabilized 5′-phosphates include, but are not limited to 5′-phosphonates, including, but not limited to 5′-vinylphosphonate, 5′-methylphosphonate, and 5′-cyclopropyl phosphonate. In certain embodiments, the stabilized phosphate group is a cyclopropyl phosphonate or an (E)-vinyl phosphonate. In certain embodiments, a terminal group comprises one or more abasic sugar moieties. In certain embodiments, a terminal group comprises one or more inverted sugar moieties and/or inverted nucleosides. In certain embodiments, a terminal group comprises one or more 2′-linked nucleosides or sugar moieties. In certain embodiments, the 2′-linked terminal group is an abasic sugar moiety. In certain embodiments, an antisense oligonucleotide has a vinylphosphonate group on the 5′-end (5′-vP). In certain particular embodiments, each antisense oligonucleotide has a vinyl phosphonate group on the 5′-end (5′-vP).

### IV. Target Nucleic Acids

### A. PLN

In certain embodiments, oligomeric compounds comprise or consist of an oligonucleotide comprising or consisting of a region that is complementary to an equal-length target region of a target nucleic acid, wherein the target nucleic acid is PLN. In certain embodiments, PLN nucleic acid has the sequence set forth in SEQ ID NO: 1 (GENBANK Accession No. NM_002667.4) or SEQ ID NO: 2 (GENBANK Accession No. NC_000006.12, truncated from nucleosides 118545001 to 118565000). In certain embodiments, contacting a cell with an oligomeric compound (e.g., a single-stranded oligomeric compound or a paired oligomeric compound e.g., an oligomeric duplex) comprising an oligonucleotide comprising or consisting of a region that is complementary to an equal-length target region of SEQ ID NOs: 1 or 2 reduces the amount of PLN RNA in the cell, and in certain embodiments reduces the amount of phospholamban protein in the cell. In certain embodiments, the oligomeric compound consists of a modified oligonucleotide. In certain embodiments, the oligomeric compound consists of a modified oligonucleotide and a conjugate group. In certain embodiments, the oligomeric compound consists of a modified oligonucleotide and one or more terminal group(s). In certain embodiments, the oligomeric compound consists of a modified oligonucleotide and a conjugate group and one or more terminal group(s).

In certain embodiments, oligomeric compounds comprise an antisense oligonucleotide comprising or consisting of a region that is complementary to a nucleobase sequence of an equal-length target region of a target PLN nucleic acid. In certain embodiments, oligomeric compounds comprise an antisense oligonucleotide comprising a region that is complementary to a nucleobase sequence of a target PLN nucleic acid, and a sense oligonucleotide comprising a region that is complementary to the nucleobase sequence of the antisense oligonucleotide or a region thereof. In certain embodiments, the target nucleic acid is an endogenous PLN RNA molecule. In certain embodiments, the target PLN nucleic acid encodes phospholamban. In certain embodiments, the target PLN nucleic acid is a precursor to a nucleic acid that encodes phospholamban. In certain such embodiments, the target PLN nucleic acid is selected from: a mature mRNA and a pre-mRNA, including intronic, exonic and untranslated regions. In certain embodiments, the target PLN RNA is a mature mRNA. In certain embodiments, the target nucleic acid is a pre-mRNA. In certain embodiments, the oligomeric compound or oligomeric duplex is an RNAi agent. In certain embodiments, antisense compounds comprise an antisense oligonucleotide comprising or consisting of a region that is complementary to a nucleobase sequence of a target PLN nucleic acid.

In certain embodiments, antisense oligonucleotides of the invention are complementary to a nucleobase sequence in a target PLN nucleic acid over the entire length of the modified oligonucleotide. In certain embodiments, antisense oligonucleotides are at least 99%, at least 95%, at least 90%, at least 85%, or at least 80% complementary to an equal length portion of the target PLN nucleic acid. In certain embodiments, antisense oligonucleotides are at least 80% complementary to a nucleobase sequence of an equal length portion of the target PLN nucleic acid over the entire length of the oligonucleotide and comprise a region that is 100% or fully complementary to a sequence in the target PLN nucleic acid.

In certain embodiments, a region of full complementarity is from 6 to 20, 10 to 18, 14 to 18, 16 to 20, or 18 to 20 nucleobases in length. In certain embodiments, the complementary region comprises or consists of at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases. In certain embodiments, the complementary region comprises or consists of at least 8, at least 9, at least 10, at least 11, at least 12, at least 13, at least 14, at least 15, at least 16, at least 17, at least 18, at least 19, at least 20, at least 21, at least 22, or 23 contiguous nucleobases. In certain embodiments, the complementary region constitutes at least 75%, at least 80%, at least 85%, at least 90%, or at least 95% of the nucleosides of the antisense oligonucleotide. In certain embodiments, the complementary region constitutes all of the nucleosides of the antisense oligonucleotide. In certain embodiments, the complementary region of the antisense oligonucleotide is at least 99%, at least 95%, at least 90%, at least 85%, or at least 80% complementary to a nucleobase sequence in the target PLN nucleic acid. In certain embodiments, the complementary region of the antisense oligonucleotide is 100% complementary to a nucleobase sequence in the target PLN nucleic acid.

In certain embodiments, antisense oligonucleotides comprise one or more mismatched nucleobases relative to the target PLN nucleic acid or portion thereof. In certain embodiments, antisense activity against the target is reduced by such mismatch, but activity against a non-target is reduced by a greater amount. Thus, in certain embodiments selectivity of the antisense oligonucleotides is improved. In certain embodiments, antisense oligonucleotides are at least 80% complementary to a nucleobase sequence of an equal length portion of the target PLN nucleic acid over the entire length of the oligonucleotide and comprise no more than one to three mismatches with the equal length portion of the target PLN nucleic acid. In certain embodiments, antisense oligonucleotides are at least 80% complementary to a nucleobase sequence in the target PLN nucleic acid over the entire length of the oligonucleotide and comprise no more than one to three mismatches with target nucleic acid, not inclusive of terminal nucleobases of the antisense oligonucleotide. In certain embodiments, antisense oligonucleotides comprise a region that is at least 80% complementary to a nucleobase sequence of an equal length portion of the target PLN nucleic acid and the region comprises no more than one to three mismatches with the equal length portion of the target PLN nucleic acid. In certain embodiments, antisense oligonucleotides are at least 80%, at least 85%, at least 90%, at least 95%, or 100% complementary to an equal length portion of the target PLN nucleic acid over the entire length of the oligonucleotide sequence. In certain embodiments, a mismatch is specifically positioned within an antisense oligonucleotide. In certain embodiments, a mismatch is at position 3, 4, 5, 6, 7, 8, 9, 10, 11, or 12 from the 5′-end of the antisense oligonucleotide. In certain embodiments, a mismatch is at position 11, 10, 9, 8, 7, 6, 5, 4, 3, or 2 from the 3′-end of the antisense oligonucleotide. In certain embodiments, a mismatch is at position 1, 2, 3, or 4 from the 5′-end of the antisense oligonucleotide. In certain embodiments, a mismatch is at position 1 and/or 2 from the 5′-end of the antisense oligonucleotide. In certain embodiments, a mismatch is at position 4, 3, 2, or 1 from the 3′-end of the antisense oligonucleotide. In certain embodiments, a mismatch is at position 1 and/or 2 from the 3′-end of the antisense oligonucleotide. In certain embodiments, 1-2 additional mismatches may be present at a terminus or at both termini of the antisense oligonucleotide.

### B. Target Nucleic Acids in Certain Tissues

In certain embodiments, oligomeric compounds comprise or consist of an oligonucleotide comprising a region that is complementary to a nucleobase sequence of an equal length portion of a PLN target nucleic acid, wherein the PLN target nucleic acid is expressed in a pharmacologically relevant tissue. In certain embodiments, the PLN target nucleic acid is expressed in the heart cells and cardiac tissues, e.g., cardiomyocytes, cardiac muscle.

### C. Oligonucleotide Sequences

Provided herein are oligomeric compounds comprising modified oligonucleotides comprising a region that is complementary to a sequence of nucleobases in a PLN nucleic acid, such as, for example, a human PLN nucleic acid, such as SEQ ID NO: 1 (GENBANK Accession No. NM_002667.4), and/or SEQ ID NO: 2 (GENBANK Accession No. NC_000006.12, truncated from nucleosides 118545001 to 118565000) and compositions comprising such oligomeric compounds. In certain embodiments, a modified oligonucleotide of the invention comprises or consists of a region having a nucleobase sequence that is at least 80%, at least 85%, at least 90%, at least 95%, or at least 99% complementary to a sequence of nucleobases in SEQ ID NOs: 1 or 2. In certain embodiments, a modified oligonucleotide of the invention comprises or consists of a region having a nucleobase sequence that is at least 80%, at least 85%, at least 90%, at least 95%, or at least 99% complementary to a nucleobase sequence comprising or consisting of a nucleobase sequence that is at least 80%, at least 85%, at least 90%, at least 95%, or at least 99% complementary to a sequence of nucleobases in SEQ ID NOs: 1 or 2. In certain embodiments, a modified oligonucleotide comprises or consists of a region having a nucleobase sequence that is 100% complementary to a sequence of nucleobases in SEQ ID NOs: 1 or 2. In certain embodiments, a modified oligonucleotide of the invention has a nucleobase sequence comprising or consisting of a nucleobase sequence that is at least 80%, at least 85%, at least 90%, at least 95%, or at least 99% complementary to a nucleobase sequence comprising or consisting of a nucleobase sequence that is 100% complementary to a sequence of nucleobases in SEQ ID NOs: 1 or 2. In certain embodiments, a modified oligonucleotide has a nucleobase sequence comprising or consisting of a nucleobase sequence that is selected from among the sequences provided in Table A, Tables B1-B2, Table B1a or B1b, Tables 1-21, Table 23, or SEQ ID NOS: 3-1016, 1027-1044, 1254-1290.

### V. Methods and Uses

### A. Antisense Activity

In certain embodiments, oligomeric compounds of the invention are capable of hybridizing to a target nucleic acid, resulting in at least one antisense activity; such oligomeric compounds and oligomeric duplexes are antisense compounds.

In certain antisense activities, hybridization of an antisense oligonucleotide to a target nucleic acid results in recruitment of a protein that cleaves the target nucleic acid. For example, in certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi agents. RNAi agents may be double-stranded (siRNA or dsRNAi) or single-stranded (ssRNA). In certain embodiments, RNAi agents are capable of RISC-mediated modulation of a target nucleic acid in a cell. In certain embodiments, such compounds reduce or inhibit the amount or activity of a target nucleic acid by 25% or more in the standard in vitro assay. In certain embodiments, RNAi agents selectively affect one or more target nucleic acid. Such RNAi agents comprise an oligonucleotide sequence having a nucleobase sequence that hybridizes to one or more target nucleic acid, resulting in one or more desired antisense activity. In certain embodiments, an RNAi agent does not hybridize to one or more non-target nucleic acid or does not hybridize to one or more non-target nucleic acid in such a way that results in significant undesired antisense activity.

In certain embodiments, provided herein are methods of decreasing, reducing and/or inhibiting PLN expression, PLN RNA levels and/or phospholamban levels and/or activity, in a cell, tissue, and/or animal (e.g., human subject) comprising contacting the cell, tissue or subject with an oligomeric compound (e.g., an oligomeric duplex) comprising or consisting of a modified antisense oligonucleotide comprising or consisting of a region having a nucleobase sequence complementary to sequence in a PLN nucleic acid (e.g., a nucleobase sequence in SEQ ID NO: 1 or SEQ ID NO: 2). In certain embodiments, the cell is a cardiac cell, cardiomyocyte, or cardiovascular smooth muscle cell. In certain embodiments, the tissue is heart, muscle (e.g., cardiac muscle). In certain embodiments, such oligomeric compound (e.g., oligomeric duplex) reduces and/or inhibits PLN expression, PLN RNA levels and/or phospholamban levels and/or activity in the heart, plasma, serum, blood or other fluid of the subject.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or animal (e.g., a human). In certain embodiments, the detectable amount of the PLN RNA may be reduced or decreased by at least 10%, at least 20%, at least 30%, at least 40%, at least 50%, at least 60%, at least 70%, at least 80%, or at least 90%, for example, compared to the amount of PLN RNA prior to contacting a cell, tissue, or animal with the oligomeric compound (e.g., an oligomeric duplex) comprising or consisting of a modified antisense oligonucleotide comprising or consisting of linked nucleosides comprising a region having a nucleobase sequence complementary to sequence in a PLN nucleic acid or compared to the amount of PLN RNA in a cell, tissue, or animal that has not been contacted with the oligomeric compound (e.g., an oligomeric duplex), e.g., a control cell, tissue or animal.

### B. Treatment, Prophylaxis

In certain embodiments, provided herein are methods of decreasing, reducing and/or inhibiting PLN expression, PLN RNA levels and/or phospholamban levels and/or activity, in a subject having, or at risk of having, a disease, disorder, condition or injury associated with PLN and/or phospholamban, such as, for example, a disease, disorder, condition or injury associated with cardiac calcium misregulation, wherein the method includes administering to the subject an oligomeric compound, modified antisense oligonucleotide, or oligomeric duplex comprising or consisting of a modified oligonucleotide comprising or consisting of a region having a nucleobase sequence complementary to a nucleobase sequence in a PLN nucleic acid, thereby inhibiting or reducing, PLN expression, PLN RNA levels and/or phospholamban levels and/or activity in the subject. In certain embodiments, administering such oligomeric compound or oligomeric duplex reduces and/or inhibits PLN expression, PLN RNA levels and/or phospholamban levels and/or activity in the heart, plasma, serum, blood and/or other body fluid of the subject. In certain embodiments, administering such oligomeric compound or oligomeric duplex reduces and/or inhibits PLN expression, PLN RNA levels and/or phospholamban levels and/or activity in the muscle, e.g., cardiac muscle, of the subject. In some instances, such oligomeric compound or oligomeric duplex is administered parenterally. In some instances, an oligomeric compound or oligomeric duplex is administered intravenously, intramuscularly, or subcutaneously. In certain embodiments, the detectable amount of the PLN RNA may be reduced by at least 10%, at least 20%, at least 30%, at least 40%, at least 50%, at least 60%, at least 70%, at least 75%, at least 80%, at least 85%, or at least 90%, for example, compared to the amount of PLN RNA prior to administering the oligomeric compound (e.g., an antisense oligonucleotide, oligomeric duplex) to the subject or compared to the amount of PLN RNA in a subject to whom the oligomeric compound has not been administered. In certain embodiments, the detectable amount of PLN RNA may be reduced by about 70% to about 85%, or about 75% to about 85%, or up to about 90%, or up to about 85%. In certain embodiments, an oligomeric compound comprising or consisting of, or an oligomeric duplex comprising, a modified antisense oligonucleotide comprising or consisting of a region having a nucleobase sequence complementary to a nucleobase sequence in SEQ ID NO: 1 or SEQ ID NO: 2 is capable of decreasing or reducing a detectable amount of a phospholamban protein in a cell, organ, e.g., the heart, tissue (e.g., muscle, e.g., cardiac muscle), plasma, serum, blood, or other fluid of the subject, when the oligomeric compound or oligomeric duplex is administered to the cell, a tissue, and/or subject. In certain embodiments, the detectable amount of the phospholamban protein may be reduced by at least 10%, at least 20%, at least 30%, at least 40%, at least 50%, at least 60%, at least 70%, at least 75%, at least 80%, at least 85%, or at least 90%, for example, compared to the amount of phospholamban protein prior to administering the oligomeric compound (e.g., an antisense oligonucleotide, oligomeric duplex) to the cell, tissue and/or subject or compared to the amount of PLN RNA in a cell, tissue, and/or subject to whom the oligomeric compound has not been administered. In certain embodiments, the detectable amount of the phospholamban protein may be reduced by about 70% to about 85%, or about 75% to about 85%, or up to about 90%, or up to about 85%.

In certain embodiments, provided herein are methods for preventing, treating, or delaying or preventing the development or progression of, diseases, disorders, conditions or injuries associated with PLN and/or phospholamban, such as, for example, a disease, disorder, condition or injury associated with cardiac calcium misregulation, wherein the method comprises administering to a subject an oligomeric compound or oligomeric duplex of the invention (e.g., a modified antisense oligonucleotide, an antisense compound) comprising or consisting of a modified oligonucleotide comprising or consisting of a region having a nucleobase sequence complementary to a nucleobase sequence in a PLN nucleic acid. Also provided are methods of ameliorating, preventing, or delaying the onset of, one or more symptoms associated with diseases, disorders, conditions or injuries associated with PLN or phospholamban, such as, for example, a disease, disorder, condition or injury associated with cardiac calcium misregulation, wherein the method comprises administering to a subject an oligomeric compound comprising or consisting of, or an oligomeric duplex comprising, a modified antisense oligonucleotide comprising or consisting of a region having a nucleobase sequence complementary to a nucleobase sequence in a PLN nucleic acid. Examples of diseases, disorders or conditions associated with PLN and/or phospholamban treatable with the compounds, compositions, and methods provided herein include, for example, a disease, disorder or condition associated with cardiac calcium misregulation (e.g., cardiovascular/cardiac injury, disease, disorder or condition as further described herein). In certain embodiments, the disease, disorder, condition or injury associated with cardiac calcium misregulation, for example, a cardiac or cardiovascular disease, disorder, condition or injury, is cardiomyopathy, arrythmia, and/or heart failure. In certain embodiments, cardiomyopathy is genetic, including PLN p.Arg14del, Arg9Cys (R9C), and Arg25Cys (R25C) mutations. In certain embodiments, cardiomyopathy is dilated cardiomyopathy (DCM). In certain embodiments, DCM is genetic, including TTN, LMNA, RBM20, SCN5A, MYH7, TNNT2, and TPM1 mutations. In certain embodiments, DCM is arrhythmogenic DCM. In certain embodiments, heart failure is heart failure with preserved ejection fraction (HFpEF), heart failure with reduced ejection fraction (HFrEF), acute heart failure, or worsening of chronic heart failure. In certain embodiments, arrhythmia is ventricular tachycardia (vtac) or ventricular fibrillation (vfib).

Cardiac dysfunction associated with cardiac calcium misregulation is associated with cardiovascular and cardiac diseases and disorders, conditions and/or injuries such as, for example, cardiomyopathy, cardiac arrythmia and heart failure. Symptoms of a cardiac disease, disorder, condition or injury include pain, hypokalemia, heart palpitations (e.g., irregular tempo, fast heartbeat, forceful heartbeat, or fluttering), chest pain, fatigue, shortness of breath, weakness, lightheadedness, dizziness, fainting episode(s), nausea, confusion, intolerance to exertion, blood clots, or a combination thereof. In certain embodiments, the cardiac disorder or cardiac injury is heart failure, a cardiomyopathy, or a cardiac arrythmia. Cardiomyopathy may be, for example, hypertrophic cardiomyopathy, dilated cardiomyopathy, restrictive cardiomyopathy, arrhythmogenic right ventricular dysplasia, or Takotsubo cardiomyopathy (broken heart syndrome). Cardiac arrythmias may include, but are not limited to, atrial or ventricular arrythmia, for example, atrial fibrillation (afib), ventricular fibrillation (vfib), or ventricular tachycardia (vtac). In some embodiments, compounds, compositions, materials and methods provided herein improve one or more indices of heart function, e.g., cardiovascular death, cardiac dilation, cardiac fibrosis, low voltage ECG, diastolic calcium uptake, ejection fraction (EF), left ventricular ejection fraction (LVEF), left ventricular end systolic volume (LVESV), left ventricular end diastolic volume (LVEDV), mitral valve flow profile, left ventricle (LV) strain, left ventricle (LV) strain rate, infarct size, heart failure hospitalization, 6 minute walk test (6MWT), the Kansas City Cardiomyopathy Questionnaire Score (KCCQS), heart rate, and heart rhythm in the subject. In certain embodiments, the compounds, methods, and pharmaceutical compositions are useful in reducing a progression of heart failure. Progression of heart failure may be classified according to the New York Heart Association classification, the American College of Cardiology/American Heart Association guidelines, and/or another method known in the art.

Thus, in certain embodiments, a method comprises administering to a subject an oligomeric compound provided herein comprising or consisting of an oligomeric duplex comprising a modified antisense oligonucleotide comprising or consisting of a region having a nucleobase sequence complementary to a sequence in a PLN nucleic acid. In certain embodiments, the subject has or is at risk for developing a cardiovascular or cardiac injury, disease, condition or disorder. In certain embodiments, the subject has or is at risk for developing cardiomyopathy, cardiac arrythmia, and/or heart failure. In certain embodiments, cardiomyopathy is genetic, including PLN p.Arg14del, Arg9Cys (R9C), and Arg25Cys (R25C) mutations. In certain embodiments, cardiomyopathy is dilated cardiomyopathy (DCM). In certain embodiments, DCM is genetic, including TTN, LMNA, RBM20, SCN5A, MYH7, TNNT2, and TPM1 mutations. In certain embodiments, DCM is arrhythmogenic DCM. In certain embodiments, heart failure is heart failure with preserved ejection fraction (HFpEF), heart failure with reduced ejection fraction (HFrEF), acute heart failure, or worsening of chronic heart failure. In certain embodiments, arrhythmia is ventricular tachycardia (vtac) or ventricular fibrillation (vfib). In certain embodiments, at least one symptom of the cardiovascular/cardiac injury, disease, condition, or disorder is ameliorated. In certain embodiments, the at least one symptom is selected from pain, hypokalemia, heart palpitations (e.g., irregular tempo, fast heartbeat, forceful heartbeat, or fluttering), chest pain, fatigue, shortness of breath, weakness, lightheadedness, dizziness, fainting episode(s), nausea, confusion, intolerance to exertion, blood clots, or a combination thereof. In certain embodiments, administration of the compound of the invention (e.g., an oligomeric compound, modified antisense oligonucleotide, or oligomeric duplex) to the subject reduces or delays the onset or progression of at least one of pain, hypokalemia, heart palpitations (e.g., irregular tempo, fast heartbeat, forceful heartbeat, or fluttering), chest pain, fatigue, shortness of breath, weakness, lightheadedness, dizziness, fainting episode(s), nausea, confusion, intolerance to exertion, or blood clots, or a combination thereof.

In certain embodiments, a method of treating cardiomyopathy, heart failure, or arrhythmia in a subject comprises administering to the subject an oligomeric compound provided herein, e.g., a modified antisense oligonucleotide, or oligomeric duplex, comprising or consisting of a modified oligonucleotide comprising or consisting of a region having a nucleobase sequence complementary to a sequence in a PLN nucleic acid, thereby treating the subject. In certain embodiments, the subject has or is at risk for developing cardiomyopathy, cardiac arrythmia, and/or heart failure. In certain embodiments, cardiomyopathy is genetic, including PLN p.Arg14del, Arg9Cys (R9C), and Arg25Cys (R25C) mutations. In certain embodiments, cardiomyopathy is dilated cardiomyopathy (DCM). In certain embodiments, DCM is genetic, including TTN, LMNA, RBM20, SCN5A, MYH7, TNNT2, and TPM1 mutations. In certain embodiments, DCM is arrhythmogenic DCM. In certain embodiments, heart failure is heart failure with preserved ejection fraction (HFpEF), heart failure with reduced ejection fraction (HFrEF), acute heart failure, or worsening of chronic heart failure. In certain embodiments, arrhythmia is ventricular tachycardia (vtac) or ventricular fibrillation (vfib). In certain embodiments, administering the oligomeric compound, modified antisense oligonucleotide, or oligomeric duplex (e.g., a therapeutically effective amount of the oligomeric compound, modified antisense oligonucleotide, or oligomeric duplex) improves cardiac function, cardiovascular death, cardiac dilation, cardiac fibrosis, low voltage ECG, diastolic calcium uptake, ejection fraction (EF), left ventricular ejection fraction (LVEF), left ventricular end systolic volume (LVESV), left ventricular end diastolic volume (LVEDV), mitral valve flow profile, left ventricle (LV) strain, left ventricle (LV) strain rate, infarct size, heart failure hospitalization, 6 minute walk test (6MWT), the Kansas City Cardiomyopathy Questionnaire Score (KCCQS), heart rate, or heart rhythm in the subject. In certain embodiments, at least one symptom of the cardiomyopathy, cardiac arrythmia and/or heart failure is ameliorated. In certain embodiments, the at least one symptom is selected from pain, hypokalemia, heart palpitations (e.g., irregular tempo, fast heartbeat, forceful heartbeat, or fluttering), chest pain, fatigue, shortness of breath, weakness, lightheadedness, dizziness, fainting episode(s), nausea, confusion, intolerance to exertion, blood clots, or a combination thereof. In certain embodiments, administration of a pharmaceutical composition comprising a compound of the invention (e.g., an oligomeric compound, modified antisense oligonucleotide, or oligomeric duplex) to the subject reduces or delays the onset or progression of at least one of pain, hypokalemia, heart palpitations (e.g., irregular tempo, fast heartbeat, forceful heartbeat, or fluttering), chest pain, fatigue, shortness of breath, weakness, lightheadedness, dizziness, fainting episode(s), nausea, confusion, intolerance to exertion, or blood clots, or a combination thereof.

In certain embodiments, a method of inhibiting expression of, or decreasing or reducing the amount of, PLN nucleic acid, such as RNA, and/or phospholamban in a subject having or at risk of a disease, injury, condition or disorder associated with PLN comprises administering to the subject an oligomeric compound, modified antisense oligonucleotide, or oligomeric duplex, any of which comprising a modified oligonucleotide comprising or consisting of a region having a nucleobase sequence complementary to a sequence of nucleobases in a PLN nucleic acid, thereby inhibiting expression of, or decreasing or reducing the amount of, PLN nucleic acid and/or PLN protein in the subject. In certain embodiments, administering the oligomeric compound, modified oligonucleotide, or oligomeric duplex inhibits expression of, or decreases or reduces the amount of, PLN nucleic acid and/or PLN protein in the heart. In certain embodiments, the subject has, or is at risk of having, cardiomyopathy, heart failure, or arrhythmia. In certain embodiments, cardiomyopathy is genetic, including PLN p.Arg14del, Arg9Cys (R9C), and Arg25Cys (R25C) mutations. In certain embodiments, cardiomyopathy is dilated cardiomyopathy (DCM). In certain embodiments, DCM is genetic, including TTN, LMNA, RBM20, SCN5A, MYH7, TNNT2, and TPM1 mutations. In certain embodiments, DCM is arrhythmogenic DCM. In certain embodiments, heart failure is heart failure with preserved ejection fraction (HFpEF), heart failure with reduced ejection fraction (HFrEF), acute heart failure, or worsening of chronic heart failure. In certain embodiments, arrhythmia is ventricular tachycardia (vtac) or ventricular fibrillation (vfib). In certain embodiments, administering the oligomeric compound, modified antisense oligonucleotide, or oligomeric duplex improves cardiac function, cardiovascular death, cardiac dilation, cardiac fibrosis, low voltage ECG, diastolic calcium uptake, ejection fraction (EF), left ventricular ejection fraction (LVEF), left ventricular end systolic volume (LVESV), left ventricular end diastolic volume (LVEDV), mitral valve flow profile, left ventricle (LV) strain, left ventricle (LV) strain rate, infarct size, heart failure hospitalization, 6 minute walk test (6MWT), the Kansas City Cardiomyopathy Questionnaire Score (KCCQS), heart rate, or heart rhythm in the subject. In certain embodiments, at least one symptom of the cardiomyopathy, cardiac arrythmia and/or heart failure is ameliorated. In certain embodiments, the at least one symptom is selected from pain, hypokalemia, heart palpitations (e.g., irregular tempo, fast heartbeat, forceful heartbeat, or fluttering), chest pain, fatigue, shortness of breath, weakness, lightheadedness, dizziness, fainting episode(s), nausea, confusion, intolerance to exertion, blood clots, or a combination thereof. In certain embodiments, administration of a pharmaceutical composition comprising a compound of the invention (e.g., a an oligomeric compound, modified antisense oligonucleotide, or oligomeric duplex) to the subject reduces or delays the onset or progression of at least one of pain, hypokalemia, heart palpitations (e.g., irregular tempo, fast heartbeat, forceful heartbeat, or fluttering), chest pain, fatigue, shortness of breath, weakness, lightheadedness, dizziness, fainting episode(s), nausea, confusion, intolerance to exertion, or blood clots, or a combination thereof.

Certain embodiments are drawn to an oligomeric compound, modified oligonucleotide, or oligomeric duplex, any of which comprising a modified antisense oligonucleotide comprising or consisting of a region having a nucleobase sequence complementary to a nucleobase sequence in a PLN nucleic acid, for use in treating a disease, disorder, condition or injury associated with PLN and/or phospholamban. In certain embodiments, the disease, disorder, condition or injury is associated with cardiac calcium misregulation, for example, a cardiac or cardiovascular disease, disorder, condition or injury (e.g., cardiomyopathy, cardiac arrythmia, heart failure). Certain embodiments provided herein are drawn to an oligomeric compound, modified oligonucleotide, or oligomeric duplex, any of which comprising a modified antisense oligonucleotide comprising or consisting of a region having a nucleobase sequence complementary to a nucleobase sequence in a PLN nucleic acid, for use in treating a disease, disorder, condition or injury associated with cardiac calcium misregulation, for example, a cardiac or cardiovascular disease, disorder, condition or injury (e.g., cardiomyopathy, cardiac arrythmia, heart failure). In certain embodiments, the disease, disorder, condition or injury is cardiomyopathy, heart failure, or arrhythmia. In certain embodiments, cardiomyopathy is genetic, including PLN p.Arg14del, Arg9Cys (R9C), and Arg25Cys (R25C) mutations. In certain embodiments, cardiomyopathy is dilated cardiomyopathy (DCM). In certain embodiments, DCM is genetic, including TTN, LMNA, RBM20, SCN5A, MYH7, TNNT2, and TPM1 mutations. In certain embodiments, DCM is arrhythmogenic DCM. In certain embodiments, heart failure is heart failure with preserved ejection fraction (HFpEF), heart failure with reduced ejection fraction (HFrEF), acute heart failure, or worsening of chronic heart failure. In certain embodiments, arrhythmia is ventricular tachycardia (vtac) or ventricular fibrillation (vfib). In certain embodiments, an oligomeric compound, modified oligonucleotide, or oligomeric duplex is for use in improving cardiac function, cardiovascular death, cardiac dilation, cardiac fibrosis, low voltage ECG, diastolic calcium uptake, ejection fraction (EF), left ventricular ejection fraction (LVEF), left ventricular end systolic volume (LVESV), left ventricular end diastolic volume (LVEDV), mitral valve flow profile, left ventricle (LV) strain, left ventricle (LV) strain rate, infarct size, heart failure hospitalization, 6 minute walk test (6MWT), the Kansas City Cardiomyopathy Questionnaire Score (KCCQS), heart rate, or heart rhythm associated with cardiomyopathy, heart failure, or arrhythmia.

Certain embodiments are drawn to an oligomeric compound, modified oligonucleotide, or oligomeric duplex, any of which comprising a modified antisense oligonucleotide comprising or consisting of a region having a nucleobase sequence complementary to a nucleobase sequence in a PLN nucleic acid, for the manufacture or preparation of a medicament for ameliorating, or delaying or preventing development or progression of a disease, disorder, condition or injury and/or for ameliorating, preventing or delaying the onset of one or more symptoms of a disease, disorder, condition or injury, wherein the disease, disorder, condition or injury is associated with cardiac calcium misregulation, for example, a cardiac or cardiovascular disease, disorder, condition or injury (e.g., cardiomyopathy, cardiac arrythmia, heart failure). In certain embodiments, the disease is cardiomyopathy, heart failure, or arrhythmia. In certain embodiments, an oligomeric compound, modified oligonucleotide, or oligomeric duplex is for the manufacture or preparation of a medicament for improving cardiac function, cardiovascular death, cardiac dilation, cardiac fibrosis, low voltage ECG, diastolic calcium uptake, ejection fraction (EF), left ventricular ejection fraction (LVEF), left ventricular end systolic volume (LVESV), left ventricular end diastolic volume (LVEDV), mitral valve flow profile, left ventricle (LV) strain, left ventricle (LV) strain rate, infarct size, heart failure hospitalization, 6 minute walk test (6MWT), the Kansas City Cardiomyopathy Questionnaire Score (KCCQS), heart rate, or heart rhythm associated with cardiomyopathy, heart failure, or arrhythmia. In certain embodiments, cardiomyopathy is genetic, including p.Arg14del, Arg9Cys (R9C), and Arg25Cys (R25C) mutations. In certain embodiments, cardiomyopathy is dilated cardiomyopathy (DCM). In certain embodiments, DCM is genetic, including TTN, LMNA, RBM20, SCN5A, MYH7, TNNT2, and TPM1 mutations. In certain embodiments, DCM is arrhythmogenic DCM. In certain embodiments, heart failure is heart failure with preserved ejection fraction (HFpEF), heart failure with reduced ejection fraction (HFrEF), acute heart failure, or worsening of chronic heart failure. In certain embodiments, arrhythmia is ventricular tachycardia (vtac) or ventricular fibrillation (vfib).

Certain embodiments are drawn to an oligomeric compound, modified oligonucleotide, or oligomeric duplex, any of which comprising a modified antisense oligonucleotide comprising or consisting of a region having a nucleobase sequence complementary to a nucleobase sequence in a PLN nucleic acid, for the manufacture or preparation of a medicament for treating a disease, disorder, condition or injury associated with cardiac calcium misregulation, for example, a cardiac or cardiovascular disease, disorder, condition or injury (e.g., cardiomyopathy, cardiac arrythmia, heart failure). In certain embodiments, cardiomyopathy is genetic, including PLN p.Arg14del, Arg9Cys (R9C), and Arg25Cys (R25C) mutations. In certain embodiments, cardiomyopathy is dilated cardiomyopathy (DCM). In certain embodiments, DCM is genetic, including TTN, LMNA, RBM20, SCN5A, MYH7, TNNT2, and TPM1 mutations. In certain embodiments, DCM is arrhythmogenic DCM. In certain embodiments, heart failure is heart failure with preserved ejection fraction (HFpEF), heart failure with reduced ejection fraction (HFrEF), acute heart failure, or worsening of chronic heart failure. In certain embodiments, arrhythmia is ventricular tachycardia (vtac) or ventricular fibrillation (vfib).

In certain embodiments, prophylactic administration of an oligomeric compound, modified antisense oligonucleotide, or oligomeric duplex or composition provided herein to a subject at risk for cardiomyopathy, heart failure, or arrhythmia, is able to prevent, ameliorate, postpone or delay a symptom and/or development or progression of cardiomyopathy progression and/or heart failure. In certain embodiments, an oligomeric compound, modified oligonucleotide, or oligomeric duplex is for the manufacture or preparation of a medicament for improving cardiac function, cardiovascular death, cardiac dilation, cardiac fibrosis, low voltage ECG, diastolic calcium uptake, ejection fraction (EF), left ventricular ejection fraction (LVEF), left ventricular end systolic volume (LVESV), left ventricular end diastolic volume (LVEDV), mitral valve flow profile, left ventricle (LV) strain, left ventricle (LV) strain rate, infarct size, heart failure hospitalization, 6 minute walk test (6MWT), the Kansas City Cardiomyopathy Questionnaire Score (KCCQS), heart rate, or heart rhythm associated with cardiomyopathy, heart failure, or arrhythmia.

In any of the methods or uses described herein, the oligomeric compound, modified oligonucleotide, oligomeric duplex or antisense compound can be any described herein.

In some embodiments an oligomeric compound or oligomeric duplex of the invention has PLN RNA and/or protein reduction activity, and, in particular embodiments, cardiac PLN RNA and/or protein reduction activity, that is comparable to or greater than the PLN RNA and/or protein reduction activity of a comparator compound, e.g., having one or two modified oligonucleotides having the same or similar nucleobase sequence as the oligonucleotide(s) of the oligomeric compound or oligomeric duplex provided herein but having different modifications and/or motifs (e.g., sugar motif, internucleoside linkage motif). In certain embodiments, the comparator compound comprises a modified oligonucleotide that is complementary to the same or a similar PLN target region as the modified oligonucleotide of an oligomeric compound or oligomeric duplex provided herein. In certain embodiments, the comparator compound comprises a modified oligonucleotide that is complementary to a target region that is different from the target region to which a modified oligonucleotide of the oligomeric compound or oligomeric duplex provided herein is complementary. In certain embodiments, a comparator compound lacks a conjugate group or includes a different conjugate group compared to an oligomeric compound or oligomeric duplex provided herein but is otherwise the same (or includes additional differences, e.g., modifications and/or motifs). In certain embodiments, a comparator compound lacks a terminal group or includes a different terminal group compared to an oligomeric compound or oligomeric duplex provided herein but is otherwise the same (or includes additional differences, e.g., modifications and/or motifs). In certain embodiments an oligomeric compound or oligomeric duplex of the invention has cardiomyocyte PLN RNA and/or protein reduction activity that is comparable to, or greater than, the cardiomyocyte PLN RNA and/or protein reduction activity of a comparator compound, e.g., having the same or similar nucleobase sequence and different modifications. Methods of detecting the level of and/or measuring the amount of PLN RNA and/or protein in a cell, organ, tissue, system or subject (e.g., animal) are described herein and/or known in the art. In some embodiments, the amount of PLN RNA is reduced by at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% in a cell (e.g., cardiomyocyte), organ (e.g., heart), tissue, system or subject (e.g., animal) that has been contacted with or administered an oligomeric compound or oligomeric duplex of the invention provided herein (or a composition comprising such oligomeric compound or oligomeric duplex) compared to a control (e.g., a cell, organ, tissue, system or subject that had not been contacted with or administered the oligomeric compound or duplex, or was contacted with or administered a control substance (e.g., PBS)). In certain embodiments, the detectable amount of PLN RNA may be reduced by about 70% to about 85%, or about 75% to about 85%, or up to about 90%, or up to about 85%. In some embodiments, the percentage of PLN RNA decrease or reduction in a cell (e.g., a cardiomyocyte), organ (e.g., a heart), tissue (e.g., muscle, cardiac muscle), system or subject (e.g., animal) contacted with or administered an oligomeric compound, oligomeric duplex or composition provided herein is 0.1% to 30% greater or less than, 0.1% to 25% greater or less than, 0.1% to 20% greater or less than, 0.1% to 15% greater or less than, 0.1% to 10% greater or less than, or 0.1% to 5% greater or less than, 0.1% to 1% greater or less than, 5% to 40% greater or less than, 5% to 35% greater or less than, 10% to 40% greater or less than, at least 5% greater than, at least 10% greater than, at least 15% greater than, at least 20% greater than, at least 25% greater than, or at least 30% greater than the percentage of PLN RNA decrease or reduction in a cell (e.g., a cardiomyocyte), organ (e.g., a heart), tissue (e.g., muscle, cardiac muscle), system or subject (e.g., animal) contacted with or administered the same concentration or dose of a comparator compound, e.g., having the same or similar nucleobase sequence and different modifications.

In certain embodiments an oligomeric compound or oligomeric duplex of the invention has greater PLN RNA and/or protein reduction activity (i.e., greater specificity of action) in a target cell/organ/tissue/system (e.g., muscle, cardiac muscle cell or cardiac cell/organ/tissue/system) than in a non-target or off-target (e.g., liver or kidney) cell/organ/tissue/system. For example, in some embodiments administration of an oligomeric compound of the invention reduces the amount or activity of target cell/organ/tissue/system (e.g., cardiac cell/organ/tissue/system) PLN RNA and/or protein at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% compared to a control and has no, or a non-significant, effect, or significantly less effect on (e.g., reduction in) the amount or activity of PLN RNA and/or protein in a different or non- or off-target cell/organ/tissue/system, e.g., liver and/or kidney cells/tissue. In certain embodiments, the detectable amount of PLN RNA and/or PLN protein may be decreased by about 70% to about 85%, or about 75% to about 85%, or up to about 90%, or up to about 85%. In some embodiments administration of an oligomeric compound of the invention reduces the amount or activity of target cell/organ/tissue/system (e.g., muscle or cardiac cell/organ/tissue/system) PLN RNA and/or protein 15%-90%, or at least 20%, or at least 25%, or at least 30%, or at least 40%, or at least 50%, or at least 60%, or at least 70%, or at least 80%, or at least 90% more than it reduces the amount or activity of PLN RNA and/or protein in liver and/or kidney cells/tissue.

In some embodiments an oligomeric compound or oligomeric duplex of the invention has PLN RNA and/or PLN protein reduction activity, and, in particular embodiments, cardiac PLN RNA and/or PLN protein reduction activity, that is comparable to or greater than the PLN RNA and/or PLN protein reduction activity of a comparator compound, e.g., having one or two modified oligonucleotides having the same or similar nucleobase sequence as the oligonucleotide(s) of the oligomeric compound or oligomeric duplex provided herein but having different modifications and/or motifs (e.g., sugar motif, internucleoside linkage motif), and/or conjugate group but that has a longer duration of action in a subject than the comparator compound. In such cases, the oligomeric compound or oligomeric duplex may be administered less frequently and/or at lower doses than a comparator compound. In some embodiments, the amount of PLN RNA and/or PLN protein is reduced by at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% in a cell (e.g., cardiomyocyte), organ (e.g., heart), tissue, system or subject (e.g., animal) that has been contacted with or administered an oligomeric compound or oligomeric duplex of the invention provided herein (or a composition comprising such oligomeric compound or oligomeric duplex) compared to a control (e.g., a cell, organ, tissue, system or subject that had not been contacted with or administered the oligomeric compound or duplex, or was contacted with or administered a control substance (e.g., PBS)). In certain embodiments, the detectable amount of PLN RNA and/or PLN protein may be reduced by about 70% to about 85%, or about 75% to about 85%, or up to about 90%, or up to about 85%.

### VI. Pharmaceutical Compositions

In certain embodiments, described herein are pharmaceutical compositions comprising one or more oligomeric compounds or oligomeric duplexes of the invention, wherein each oligomeric compound or duplex comprises or consists of a modified oligonucleotide. In certain embodiments, the one or more oligomeric compounds or oligomeric duplex each consists of or comprises an antisense compound. In certain embodiments, a pharmaceutical composition comprises a pharmaceutically acceptable diluent or carrier. In certain embodiments, a pharmaceutical composition comprises or consists of a sterile saline solution and one or more compound or duplex of the invention. In certain embodiments, the sterile saline is pharmaceutical grade saline. In certain embodiments, a pharmaceutical composition comprises or consists of one or more compound or duplex of the invention and sterile water. In certain embodiments, the sterile water is pharmaceutical grade water. In certain embodiments, a pharmaceutical composition comprises or consists of one or more compound or duplex of the invention and phosphate-buffered saline (PBS). In certain embodiments, sterile PBS is pharmaceutical grade PBS.

In certain embodiments, a pharmaceutical composition comprises an oligomeric compound or oligomeric duplex comprising or consisting of a modified oligonucleotide; and sterile saline. In certain such embodiments, a pharmaceutical composition consists of such oligomeric compound or oligomeric duplex and sterile saline. In certain embodiments, a pharmaceutical composition consists essentially of such oligomeric compound or oligomeric duplex and sterile saline. In certain embodiments, the sterile saline is sterile PBS. In certain embodiments, the sterile saline is pharmaceutical grade.

In certain embodiments, a pharmaceutical composition comprises an oligomeric compound or oligomeric duplex comprising or consisting of a modified oligonucleotide; and sterile saline. In certain such embodiments, a pharmaceutical composition consists of such modified oligonucleotide or oligomeric duplex and sterile saline. In certain embodiments, a pharmaceutical composition consists essentially of such modified oligonucleotide and sterile saline. In certain embodiments, the sterile saline is sterile PBS. In certain embodiments, the sterile saline is pharmaceutical grade.

In certain embodiments, a pharmaceutical composition comprises an oligomeric compound or oligomeric duplex comprising or consisting of a modified oligonucleotide and sterile saline. In certain embodiments, a pharmaceutical composition consists of an oligomeric compound or oligomeric duplex comprising or consisting of a modified oligonucleotide and sterile saline. In certain embodiments, a pharmaceutical composition consists essentially of an oligomeric compound or oligomeric duplex comprising or consisting of a modified oligonucleotide and sterile saline. In certain embodiments, the sterile saline is sterile PBS. In certain embodiments, the sterile saline is pharmaceutical grade.

In certain embodiments, pharmaceutical compositions comprise one or more oligomeric compound or oligomeric duplex of the invention and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, an oligomeric compound or oligomeric duplex of the invention may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound or oligomeric duplex of the invention encompass any pharmaceutically acceptable salts of the compound or duplex, esters of the compound or duplex, or salts of such esters. As used herein “pharmaceutically acceptable salts” means physiologically and pharmaceutically acceptable salts of compounds. Pharmaceutically acceptable salts retain the desired biological activity of the parent compound and do not impart undesired toxicological effects thereto. In certain embodiments, pharmaceutical compositions comprising an oligomeric compound or oligomeric duplex of the invention comprising one or more oligonucleotide, upon administration to a subject, including a human, are capable of providing (directly or indirectly) the biologically active metabolite or residue thereof. Accordingly, for example, the disclosure is also drawn to pharmaceutically acceptable salts of oligomeric compounds or oligomeric duplexes of the invention, prodrugs, pharmaceutically acceptable salts of such prodrugs, and other bioequivalents. In certain embodiments, pharmaceutically acceptable salts comprise inorganic salts, such as monovalent or divalent inorganic salts. Suitable pharmaceutically acceptable salts include, but are not limited to, sodium, potassium, calcium, and magnesium salts.

In certain embodiments, oligomeric compounds or oligomeric duplexes of the invention are lyophilized and isolated, e.g., as sodium salts. In certain embodiments, a sodium salt of a compound or duplex of the invention is mixed with a pharmaceutically acceptable diluent. In certain embodiments, the pharmaceutically acceptable diluent comprises sterile saline, sterile water, or PBS. In certain embodiments, a sodium salt of an oligomeric compound or oligomeric duplex of the invention is mixed with PBS.

Lipid moieties have been used in nucleic acid therapies in a variety of methods. In certain methods, a nucleic acid, such as an oligomeric compound or oligomeric duplex of the invention comprising a nucleic acid, is introduced into preformed liposomes or lipoplexes made of mixtures of cationic lipids and neutral lipids. In certain methods, nucleic acid complexes with mono- or poly-cationic lipids are formed without the presence of a neutral lipid. In certain embodiments, a lipid moiety is selected to increase distribution of a pharmaceutical compound to a particular cell or tissue. In certain embodiments, a lipid moiety is selected to increase distribution of a pharmaceutical compound to fat tissue. In certain embodiments, a lipid moiety is selected to increase distribution of a pharmaceutical compound to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more compounds of the present invention to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), etc.). In certain of such embodiments, a pharmaceutical composition comprises a carrier or diluent and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, diluents, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, certain compounds disclosed herein act as acids. Although such compounds may be drawn or described in protonated (free acid) form, or ionized and in association with a cation (salt) form, aqueous solutions of such compounds exist in equilibrium among such forms. For example, a phosphodiester linkage of an oligonucleotide in aqueous solution exists in equilibrium among free acid, anion and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” herein is intended to include all such forms. Drawn structures necessarily depict a single form. Nevertheless, unless otherwise indicated, such drawings are likewise intended to include corresponding forms. Herein, a structure depicting the free acid of a compound followed by the term “or a pharmaceutically acceptable salt thereof” expressly includes all such forms that may be fully or partially protonated/de-protonated/in association with a cation or a combination of cations. In certain embodiments, one or more specific cation is identified. The cations include, but are not limited to, sodium, potassium, calcium, and magnesium. In certain embodiments, a structure depicting the free acid of a compound followed by the term “or a pharmaceutically acceptable salt thereof” expressly includes all such forms that may be fully or partially protonated/de-protonated/in association with one or more cations selected from sodium, potassium, calcium, and magnesium.

In certain embodiments, oligomeric compounds, modified oligonucleotides, or oligomeric duplexes of the invention are in aqueous solution with sodium. In certain embodiments, oligomeric compounds, modified oligonucleotides or oligomeric duplexes of the invention are in aqueous solution with potassium. In certain embodiments, oligomeric compounds, modified oligonucleotides or oligomeric duplexes of the invention are in PBS. In certain embodiments, oligomeric compounds, modified oligonucleotides or oligomeric duplexes of the invention are in water. In certain such embodiments, the pH of a solution is adjusted with NaOH and/or HCl to achieve a desired pH.

Herein, certain specific doses are described. A dose may be in the form of a dosage unit. For clarity, a dose (or dosage unit) of a compound of the invention (e.g., modified oligonucleotide, oligomeric duplex, antisense compound) in milligrams indicates the mass of the free acid form of the compound. As described herein, in aqueous solution, the free acid is in equilibrium with anionic and salt forms. However, for the purpose of calculating dose, it is assumed that the compound of the invention (e.g., modified oligonucleotide, oligomeric duplex, antisense compound) exists as a solvent-free, sodium-acetate free, anhydrous, free acid. In certain embodiments, where a compound of the invention (e.g., modified oligonucleotide, oligomeric duplex, antisense compound) is in solution comprising sodium (e.g., saline), the compound may be partially or fully de-protonated and in association with sodium ions. However, the mass of the protons is nevertheless counted toward the weight of the dose, and the mass of the sodium ions is not counted toward the weight of the dose. When a compound of the invention comprises a conjugate group, the mass of the conjugate group is included in calculating the dose of such compound. If the conjugate group also has an acid, the conjugate group is likewise assumed to be fully protonated for the purpose of calculating dose.

### VII. Compounds

Table A includes nucleobase sequences for oligomeric compounds and oligomeric duplexes that were previously described in International Patent Application Publication No. WO/2022/173976. Each of those oligomeric duplexes contains an antisense sequence that is complementary to a sequence in a human PLN nucleic acid and a sense sequence that is complementary to the antisense sequence. Each of the antisense oligonucleotides having the nucleobase sequences listed in Table A is described in International Patent Application Publication No. WO/2022/173976 as having a sugar motif (from 5′ to 3′) of: yfyfyfyfyfyfyfyfyfyfyyy, and each of the sense oligonucleotides having the nucleobase sequences listed in Table A is described in International Patent Application Publication No. WO/2022/173976 as having a sugar motif (from 5′ to 3′) of: fyfyfyfyfyfyfyfyfyfyf, wherein each “y” represents a 2′-OMe sugar moiety and each “f” represents a 2′-fluoro ribosyl moiety.

In certain embodiments, provided herein are reduced fluorine content oligomeric compounds and oligomeric duplexes. In some embodiments, a reduced fluorine content oligomeric compound comprises an oligonucleotide (e.g., an antisense oligonucleotide) which has a nucleobase sequence complementary to a sequence in a PLN nucleic acid, e.g., a human PLN nucleic acid, or an oligonucleotide (e.g., a sense oligonucleotide) which has a nucleobase sequence complementary to a sequence of an oligonucleotide which has a nucleobase sequence complementary to a sequence in a PLN nucleic acid, e.g., a human PLN nucleic acid. In some embodiments, a reduced fluorine content oligomeric duplex comprises a first modified oligonucleotide (e.g., an antisense oligonucleotide), which has a nucleobase sequence complementary to a sequence of a PLN nucleic acid (e.g., human PLN nucleic acid), and a second modified oligonucleotide (e.g., a sense oligonucleotide), which has a nucleobase sequence complementary to a sequence of the first modified oligonucleotide. In some embodiments, the reduced fluorine content oligomeric compounds and oligomeric duplexes provided herein, which may be preferable to compounds containing more fluorine atoms due to improved properties, e.g., decreased off-target actions and/or improved tolerability, have PLN RNA and/or protein reduction activity that is comparable to or greater than that of a comparator compound containing more fluorine atoms (e.g., a compound having 50% or more, 45% or more, or 40% or more fluorine-containing nucleosides). In some embodiments, an oligomeric compound or oligomeric duplex having reduced fluorine content provided herein comprises a modified oligonucleotide or a first modified oligonucleotide (e.g., antisense oligonucleotide) which has a nucleobase sequence complementary to a sequence in a PLN nucleic acid, having reduced fluorine content (e.g., fewer than 45%, fewer than 40%, fewer than 35%, fewer than 30%, fewer than 25%, fewer than 20%, fewer than 15%, or fewer than 10% of nucleosides comprising a fluorine atom, e.g., a modified sugar moiety containing fluorine) of nucleosides comprising a fluorine atom, such as e.g., a modified sugar moiety containing fluorine) and/or a modified oligonucleotide or a second modified oligonucleotide (e.g., sense oligonucleotide) comprising or consisting of a region that has a nucleobase sequence complementary to the first oligonucleotide, or to a sequence that is complementary to a sequence in a PLN nucleic acid, having reduced fluorine content (e.g., fewer than 45%, fewer than 40%, fewer than 35%, fewer than 30%, fewer than 25%, fewer than 20%, fewer than 15%, or fewer than 10% of nucleosides comprising a fluorine atom, e.g., a modified sugar moiety containing fluorine). In certain embodiments, an oligomeric compound or oligomeric duplex having reduced fluorine content provided herein comprises a modified oligonucleotide or first modified oligonucleotide (e.g., antisense oligonucleotide) having reduced fluorine content (e.g., fewer than 45%, fewer than 40%, fewer than 35%, fewer than 30%, fewer than 25%, fewer than 20%, or fewer than 15% of nucleosides comprising a fluorine atom, e.g., a modified sugar moiety containing fluorine). In certain such embodiments, the second modified oligonucleotide (e.g., sense oligonucleotide) of such oligomeric duplexes has a reduced fluorine content (e.g., fewer than 45%, fewer than 40%, fewer than 35%, fewer than 30%, fewer than 25%, fewer than 20%, fewer than 15%, or fewer than 10% of nucleosides comprising a fluorine atom, e.g., a modified sugar moiety containing fluorine). In certain embodiments, an oligomeric compound or oligomeric duplex provided herein comprises an oligonucleotide or first modified oligonucleotide (e.g., an antisense oligonucleotide) having reduced fluorine content (e.g., fewer than 45%, fewer than 40%, fewer than 35%, fewer than 30%, fewer than 25%, fewer than 20%, fewer than 15%, or fewer than 10% of nucleosides comprising a fluorine atom, e.g., a modified sugar moiety containing fluorine) comprising or consisting of a region having a nucleobase sequence that is at least 85%, at least 90%, at least 95%, or at least 99% complementary to an equal length portion of a PLN nucleic acid (e.g., a sequence in SEQ ID NO: 1). In certain embodiments, an oligomeric compound or oligomeric duplex provided herein comprises an oligonucleotide or a first modified oligonucleotide (e.g., an antisense oligonucleotide) having reduced fluorine content (e.g., fewer than 45%, fewer than 40%, fewer than 35%, fewer than 30%, fewer than 25%, fewer than 20%, fewer than 15%, or fewer than 10% of nucleosides comprising a fluorine atom, e.g., a modified sugar moiety containing fluorine) and having a nucleobase sequence selected from among the sequences provided in Table A, Table B1 or SEQ ID NOS: 3-314, 627-782, 939-976, 1033-1038, 1254-1255, and 1258-1287. In particular embodiments, such oligomeric compounds or oligomeric duplexes comprise an oligonucleotide or second modified oligonucleotide (e.g., a sense oligonucleotide), comprising or consisting of a region having a nucleobase sequence, e.g., a sequence complementary to the first modified oligonucleotide, selected from among the sequences provided in Table A, Table B2 or SEQ ID NOS: 315-626, 783-938, 977-1016, 1027-1032, 1256-1257, and 1288-1290. In some such embodiments, the modified oligonucleotide or second modified oligonucleotide has a reduced fluorine content (e.g., fewer than 40%, fewer than 35%, fewer than 30%, fewer than 25%, fewer than 20%, fewer than 15%, or fewer than 10% of nucleosides comprising a fluorine atom, e.g., a modified sugar moiety containing fluorine). In certain embodiments, an oligomeric compound or oligomeric duplex provided herein comprises an oligonucleotide or a first modified oligonucleotide (e.g., an antisense oligonucleotide) having reduced fluorine content (e.g., fewer than 45%, fewer than 40%, fewer than 35%, fewer than 30%, fewer than 25%, fewer than 20%, fewer than 15%, or fewer than 10% of nucleosides comprising a fluorine atom, e.g., a modified sugar moiety containing fluorine) and having a nucleobase sequence selected from among the sequences of SEQ ID NOS: 29,171,176, 967, 968, 971, 972, 974, 975, 1033-1038, 1254, 1255, and 1258-1287. In particular embodiments, such oligomeric compounds or oligomeric duplexes comprise an oligonucleotide or second modified oligonucleotide (e.g., a sense oligonucleotide), comprising or consisting of a region having a nucleobase sequence, e.g., a sequence complementary to the first modified oligonucleotide, selected from among the sequences of SEQ ID NOS: 341, 483, 488, 1011-1016, 1027-1032, 1256-1257, and 1288-1290. In some such embodiments, the modified oligonucleotide or second modified oligonucleotide has a reduced fluorine content (e.g., fewer than 40%, fewer than 35%, fewer than 30%, fewer than 25%, fewer than 20%, fewer than 15%, or fewer than 10% of nucleosides comprising a fluorine atom, e.g., a modified sugar moiety containing fluorine).

In some embodiments, an oligomeric compound or oligomeric duplex having reduced fluorine content provided herein comprises a conjugate group. In some such embodiments of oligomeric duplexes provided herein, the conjugate group is attached to the first (e.g., antisense) or second (e.g., sense) modified oligonucleotide of the oligomeric duplex. In some embodiments, the conjugate group is attached to the 5′- or 3′-end of the modified oligonucleotide of an oligomeric compound or of the first or second modified oligonucleotide of an oligomeric duplex, or the 5′- or 3′-terminal nucleoside of the modified oligonucleotide of an oligomeric compound or of the first or second modified oligonucleotide of an oligomeric duplex. In particular embodiments, the conjugate group is attached to the second modified oligonucleotide (e.g., sense oligonucleotide), for example, the 5′- or 3′-terminal nucleoside of the second modified oligonucleotide, of an oligomeric duplex. In particular embodiments, the conjugate group is attached to the 5′-terminal nucleoside of the second modified oligonucleotide. In some embodiments, the conjugate group comprises an active drug substance, a small molecule drug, an aliphatic chain, a lipid, a peptide, a protein, a hydrocarbon, a polyamine, a polyamide, a polyether, a thioether, an aptamer, an antibody, an antibody fragment, a vitamin, a fatty acid, a carbohydrate, an intercalator, a reporter molecule, or an alkyl moiety, e.g., a C22 alkyl, C20 alkyl, C17 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, or C5 alkyl, wherein the alkyl chain optionally has one or more unsaturated bonds. In some embodiments, the conjugate group comprises a 6-palmitamidohexyl moiety, 6-ammnohexyl, or a 2-(hydroxymethyl)-6-palmitamidohexyl moiety. In some embodiments, the conjugate moiety of the conjugate group has affinity for a transferrin receptor, e.g., a type 1 transferrin receptor, e.g., a peptide, polypeptide, protein, and low molecular weight molecule. Examples of such conjugate moieties include, but are not limited to, a transferrin receptor ligand, an antibody, a fragment of an antibody, and a drug, any of which recognizes and/or binds to a transferrin receptor. In some embodiments, the conjugate moiety is a bicycle peptide compound, such as, for example, a bicycle ligand that binds to a transferrin receptor, e.g., a type 1 transferrin receptor, including those previously described in International Patent Application Publication No. WO2022/101633, which is incorporated herein by reference, and those previously described in International Patent Application Publication No. WO2023/056388, which is hereby incorporated by reference. In some embodiments, the bicycle ligand comprises an amino acid sequence of SEQ ID NO: 1045, wherein each cysteine forms a covalent bond with the molecular scaffold 1,1′,1″-(1,3,5-triazinane-1,3,5-triyl)tris(2-bromoethanone) (TATB). In some embodiments, a bicycle ligand or a conjugate group comprising a bicycle ligand has the following structure:

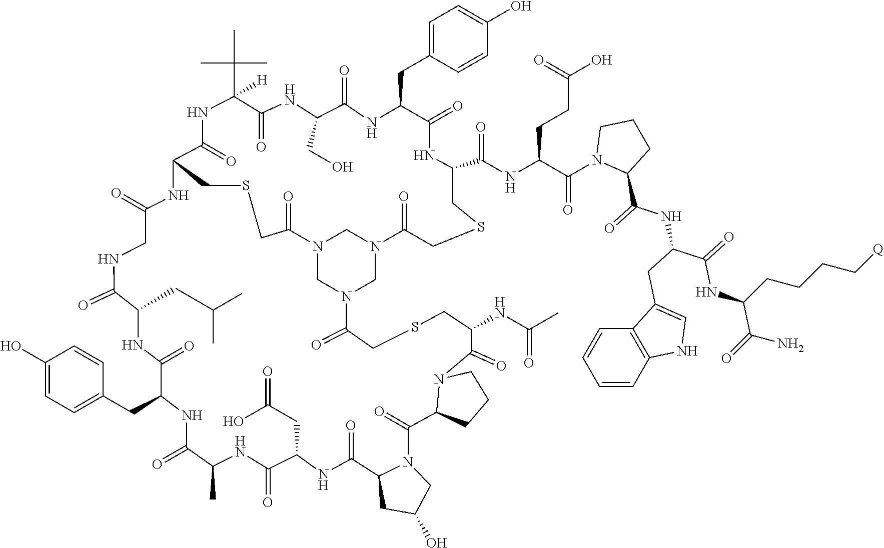

or a salt thereof, wherein Q is N₃ (BCY17901, SEQ ID NO: 1045), NH₂ (BCY21757, SEQ ID NO: 1202), SH (BCY21758, SEQ ID NO: 1203), a conjugate linker, or a conjugate linker covalently connected to the modified oligonucleotide or the first modified oligonucleotide (e.g., antisense oligonucleotide) or second modified oligonucleotide (e.g., sense oligonucleotide). In some embodiments, the conjugate group has the following structure:

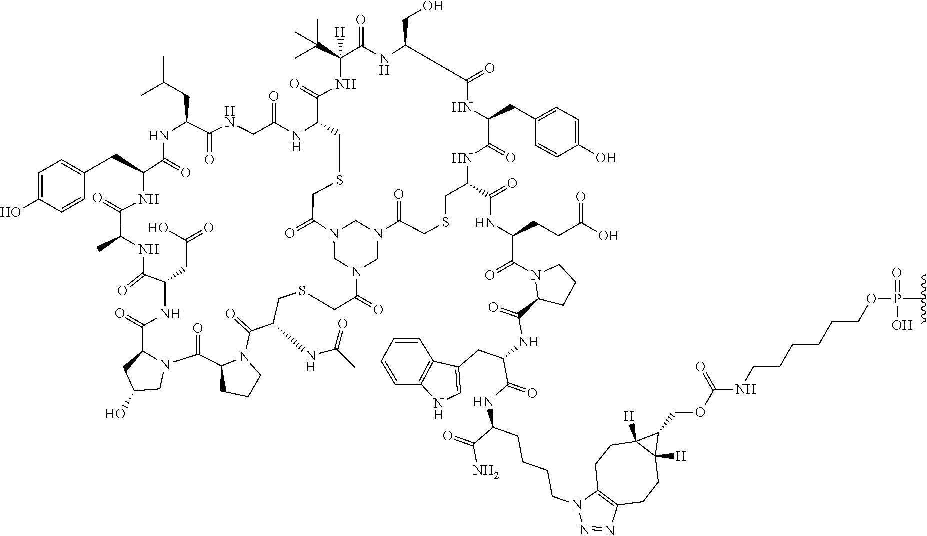 [SMILES: *P(=O)(O)OCCCCCCNC(=O)OC[C@@H]1[C@@H]2CCc3nnn(CCCC[C@H](NC(=O)[C@H](Cc4c[nH]c5ccccc45)NC(=O)[C@@H]4CCCN4C(=O)[C@H](CCC(=O)O)NC(=O)[C@@H]4CSCC(=O)N5CN6CN(C5)C(=O)CSC[C@H](NC(C)=O)C(=O)N5CCC[C@H]5C(=O)N5C[C@H](O)C[C@H]5C(=O)N[C@@H](CC(=O)O)C(=O)N[C@@H](C)C(=O)N[C@@H](Cc5ccc(O)cc5)C(=O)N[C@@H](CC(C)C)C(=O)NCC(=O)N[C@@H](CSCC6=O)C(=O)N[C@@H](C(C)(C)C)C(=O)N[C@@H](CO)C(=O)N[C@@H](Cc5ccc(O)cc5)C(=O)N4)C(N)=O)c3CC[C@@H]21]

and is attached to the modified oligonucleotide of the oligomeric compound or the first modified oligonucleotide (e.g., antisense oligonucleotide) or the second modified oligonucleotide (e.g., sense oligonucleotide) of the oligomeric duplex through a phosphodiester bond, e.g., through a phosphodiester bond with the 5′-terminal nucleoside of the modified oligonucleotide of the oligomeric compound or the second modified oligonucleotide of the oligomeric duplex. In some embodiments, the conjugate group has the following structure:

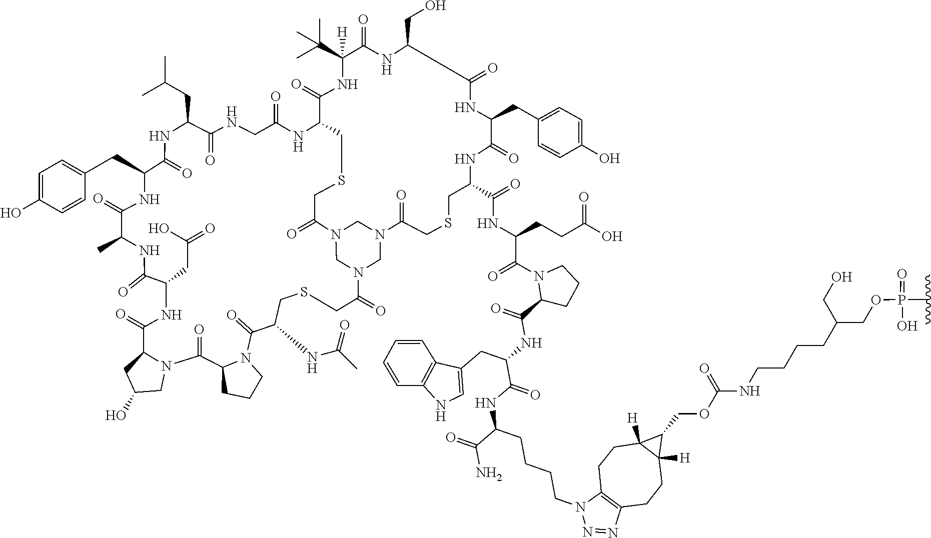 [SMILES: *P(=O)(O)OCC(CO)CCCCNC(=O)OC[C@@H]1[C@@H]2CCc3nnn(CCCC[C@H](NC(=O)[C@H](Cc4c[nH]c5ccccc45)NC(=O)[C@@H]4CCCN4C(=O)[C@H](CCC(=O)O)NC(=O)[C@@H]4CSCC(=O)N5CN6CN(C5)C(=O)CSC[C@H](NC(C)=O)C(=O)N5CCC[C@H]5C(=O)N5C[C@H](O)C[C@H]5C(=O)N[C@@H](CC(=O)O)C(=O)N[C@@H](C)C(=O)N[C@@H](Cc5ccc(O)cc5)C(=O)N[C@@H](CC(C)C)C(=O)NCC(=O)N[C@@H](CSCC6=O)C(=O)N[C@@H](C(C)(C)C)C(=O)N[C@@H](CO)C(=O)N[C@@H](Cc5ccc(O)cc5)C(=O)N4)C(N)=O)c3CC[C@@H]21]

and is attached to the modified oligonucleotide of the oligomeric compound or the first modified oligonucleotide (e.g., antisense oligonucleotide) or the second modified oligonucleotide (e.g., sense oligonucleotide) of the oligomeric duplex through a phosphodiester bond, e.g., through a phosphodiester bond with the 3′-terminal nucleoside of the modified oligonucleotide of the oligomeric compound or the second modified oligonucleotide of the oligomeric duplex.

As noted herein, in certain embodiments, an oligomeric compound or oligomeric duplex provided herein contains a modified oligonucleotide or a first modified oligonucleotide (e.g., antisense oligonucleotide) comprising or consisting of a sequence of linked nucleosides having reduced fluorine content. In some embodiments, fewer than 40% of the nucleosides of the modified oligonucleotide or first modified oligonucleotide (e.g., antisense oligonucleotide) contain a fluorine atom, e.g., a modified sugar moiety containing fluorine. In certain embodiments, fewer than 35%, fewer than 30%, fewer than 25%, fewer than 20%, fewer than 15%, fewer than 14%, fewer than 13%, fewer than 12%, fewer than 10%, fewer than 5%, or fewer than 1% of the nucleosides in the modified oligonucleotide or first modified oligonucleotide (e.g., antisense oligonucleotide) contain a fluorine atom (e.g., a modified sugar moiety containing fluorine). In certain embodiments, such an oligomeric compound or oligomeric duplex provided herein contains a second modified oligonucleotide, or a second modified sense oligonucleotide (e.g., sense oligonucleotide) comprising or consisting of linked nucleosides having reduced fluorine content. In some embodiments, fewer than 40% of the nucleosides of the modified oligonucleotide or second modified oligonucleotide contain a fluorine atom, e.g., a modified sugar moiety containing fluorine. In certain embodiments, fewer than 35%, fewer than 30%, fewer than 25%, fewer than 20%, fewer than 15%, fewer than 14%, fewer than 13%, fewer than 12%, fewer than 10%, fewer than 5%, or fewer than 1% of the nucleosides in the second modified oligonucleotide or sense modified oligonucleotide contain a fluorine atom (e.g., a modified sugar moiety containing fluorine). In particular embodiments, the total fluorine content of the oligomeric compound or oligomeric duplex is reduced (e.g., less than 40% of the total nucleobases in the compound comprising a fluorine atom, a modified sugar moiety containing fluorine). In certain embodiments, less than 35%, less than 30%, less than 25%, less than 20%, less than 15%, less than 14%, less than 13%, less than 12%, less than 10%, less than 5%, or less than 1% of the nucleosides in the oligomeric compound or oligomeric duplex having a reduced fluorine content contain a fluorine atom (e.g., a modified sugar moiety containing fluorine). In some embodiments, no more than 8, no more than 7, no more than 6, no more than 5, no more than 4, no more than 2 or no more than 1 nucleoside(s) in an oligomeric compound or oligomeric duplex having reduced fluorine content provided herein contain a fluorine atom, e.g., a modified sugar moiety containing fluorine.

In some embodiments, an oligomeric compound or oligomeric duplex having a reduced fluorine content provided herein has a modified oligonucleotide or a first modified oligonucleotide (e.g., antisense oligonucleotide) containing one or more nucleosides containing a fluorine atom (e.g., a modified sugar moiety containing fluorine). In some embodiments, such an oligomeric compound or oligomeric duplex provided herein has a modified oligonucleotide or a first modified oligonucleotide (e.g., antisense oligonucleotide) containing one, two, three or four nucleosides containing a fluorine atom. In some embodiments, the nucleosides of the modified oligonucleotide or a first modified oligonucleotide that contain a fluorine atom contain a modified sugar moiety containing a fluorine atom, e.g., a 2′-fluoro sugar moiety, or sugar surrogate containing a fluorine atom, e.g., a 3′-fluoro-hexitol sugar moiety. In some embodiments, the modified oligonucleotide or first modified oligonucleotide (e.g., antisense oligonucleotide) of an oligomeric compound or oligomeric duplex having reduced fluorine content provided herein does not contain any fluorine atoms. In certain embodiments, no more than three nucleosides, or no more than two nucleosides, in the modified oligonucleotide or first modified oligonucleotide (e.g., antisense oligonucleotide) contain a fluorine atom (e.g., a modified sugar moiety containing fluorine). In certain embodiments, no more than three nucleosides in the modified oligonucleotide or first modified oligonucleotide (e.g., antisense oligonucleotide) between and including the second and twenty-first nucleosides counting from the 5′ terminus of the modified oligonucleotide or first modified oligonucleotide contain a fluorine atom (e.g., a modified sugar moiety containing fluorine). In particular embodiments, no more than two nucleosides in the modified oligonucleotide or first modified oligonucleotide (e.g., antisense oligonucleotide) between and including the third and twenty-first nucleosides counting from the 5′ terminus of the modified oligonucleotide or first modified oligonucleotide contain a fluorine atom (e.g., a modified sugar moiety containing fluorine). In some embodiments, at least 85%, or at least 90%, or at least 95%, or at least 99%, or all of the nucleosides of the modified oligonucleotide or first modified oligonucleotide (e.g., antisense oligonucleotide) contain a 2′-modified sugar moiety and one or more of the nucleosides contain a fluorine atom (e.g., a 2′-fluoro sugar moiety). In some such embodiments, one or more of the nucleosides contain a 2′-OMe sugar moiety or a 2′-MOE sugar moiety. In some embodiments, any of the nucleosides that do not contain a fluorine atom independently contain a 2′-OMe sugar moiety or a 2′-MOE sugar moiety. In some embodiments, the 5′- and/or 3′-terminal nucleosides of the modified oligonucleotide or first modified oligonucleotide (e.g., antisense oligonucleotide) comprise a 2′-MOE sugar moiety. In some embodiments, the nucleoside immediately 5′ of the 3′-terminal nucleoside of the modified oligonucleotide or first modified oligonucleotide (e.g., antisense oligonucleotide) comprises a 2′-MOE sugar moiety. In particular embodiments, the 5′- and 3′-terminal nucleosides of the modified oligonucleotide or first modified oligonucleotide comprise a 2′-MOE sugar moiety and the nucleoside immediately 5′ of the 3′-terminal nucleoside of the modified oligonucleotide or first modified oligonucleotide comprises a 2′-MOE sugar moiety. In some embodiments, at least 10%, at least 20%, at least 30%, at least 40%, at least 50% or at least 60%, or more of the nucleosides of the modified oligonucleotide or first modified oligonucleotide (e.g., antisense oligonucleotide) comprise a 2′-OMe sugar moiety. In particular embodiments, the 3′-terminal nucleoside of the modified oligonucleotide or first modified oligonucleotide (e.g., antisense oligonucleotide) comprises a 2′-OMe sugar moiety and/or the nucleoside immediately 5′ of the 3′-terminal nucleoside of the modified oligonucleotide or first modified oligonucleotide comprises a 2′-OMe sugar moiety.

In some embodiments, an oligomeric compound or oligomeric duplex having a reduced fluorine content provided herein has a modified oligonucleotide or first modified oligonucleotide (e.g., antisense oligonucleotide) containing no more than four nucleosides containing a fluorine atom (e.g., a modified sugar moiety containing fluorine). In some such embodiments, the number of nucleosides in the modified oligonucleotide or first modified oligonucleotide is 23, 22, or 21. In some such embodiments, the nucleobase sequence of the modified oligonucleotide or first modified oligonucleotide (e.g., antisense oligonucleotide) comprises or consists of a nucleobase sequence selected from among the sequences provided in Table A, Table B1 or SEQ ID NOS: 3-314, 627-782, 939-976, 1033-1038, 1254-1255, and 1258-1287. Examples of sugar motifs for such a modified oligonucleotide or first modified oligonucleotide (e.g., antisense oligonucleotide) of an oligomeric compound or oligomeric duplex provided herein include, but are not limited to (from 5′ to 3′): efyyyfyyyyyyyfyfyyyyyee, efyyyfyyyyyyyfyfyyyyyyy, efyyyfyyyyyyyfyfyyyyye, e[FHNA]yyyfyyyyyyyfyfyyyyyee, e[FHNA]yyyfyyyyyyyfyfyyyyyyy, e[FHNA]yyyfyyyyyyyfyfyyyyye, e[FHNA]yyyfyyyyyyyfyfyyyyy, efyyyfyyyyyyyfyfyyyyyey, efyyyfyyyyyyyfyfyyyyye, efyyyfyyyyyyyfyfyyyee, and efyyyfyyyyyyyfyfyyyyy, wherein each “y” represents a 2′-OMe sugar moiety, each “e” represents a 2′-MOE sugar moiety, each “f” represents a 2′-fluoro sugar moiety, and each “[FHNA]” represents a 3′-fluoro-hexitol sugar moiety. In some embodiments, an oligomeric compound or oligomeric duplex having a reduced fluorine content provided herein has a modified oligonucleotide or first modified oligonucleotide (e.g., antisense oligonucleotide) containing no more than three nucleosides containing a fluorine atom (e.g., a modified sugar moiety containing fluorine). In some such embodiments, the number of nucleosides in the modified oligonucleotide or first modified oligonucleotide is 23, 22, or 21. In some such embodiments, the nucleobase sequence of the modified oligonucleotide or first modified oligonucleotide (e.g., antisense oligonucleotide) comprises or consists of a nucleobase sequence selected from among the sequences provided in Table A, Table B1 or SEQ ID NOS: 3-314, 627-782, 939-976, 1033-1038, 1254-1255, and 1258-1287. In some such embodiments, the nucleobase sequence of the modified oligonucleotide or first modified oligonucleotide (e.g., antisense oligonucleotide) comprises or consists of a nucleobase sequence selected from among the nucleobase sequences of SEQ ID NOS: 29,171,176, 967, 968, 971, 972, 974, 975, 1033-1038, 1254, 1255, and 1258-1287. Examples of sugar motifs for such a modified oligonucleotide or first modified oligonucleotide of an oligomeric compound or oligomeric duplex provided herein include, but are not limited to, (from 5′ to 3′): efyyyyyyyyyyyfyfyyyyyee, efyyyyyyyyyyyfyfyyyyyyy, efyyyyyyyyyyyfyfyyyyye, efyyyyyyyyyyyfyfyyyyy, efyyyfyyyyyyyfyyyyyyyyy, and efyyyfyyyyyyyfyyyyyyyee, wherein each “y” represents a 2′-OMe sugar moiety, each “e” represents a 2′-MOE sugar moiety, and each “f” represents a 2′-fluoro sugar moiety. In some embodiments, an oligomeric compound or oligomeric duplex having a reduced fluorine content provided herein has a modified oligonucleotide or first modified oligonucleotide (e.g., antisense oligonucleotide) containing no more than two nucleosides containing a fluorine atom (e.g., a modified sugar moiety containing fluorine). In some such embodiments, the number of nucleosides in the modified oligonucleotide or first modified oligonucleotide is 23, 22, or 21. In some such embodiments, the nucleobase sequence of the modified oligonucleotide or first modified oligonucleotide (e.g., antisense oligonucleotide) comprises or consists of a nucleobase sequence selected from among the sequences provided in Table A, Table B1 or SEQ ID NOS: 3-314, 627-782, 939-976, 1033-1038, 1254-1255, and 1258-1287. In some such embodiments, the nucleobase sequence of the modified oligonucleotide or first modified oligonucleotide (e.g., antisense oligonucleotide) comprises or consists of a nucleobase sequence selected from among the nucleobase sequences of SEQ ID NOS: 29, 171, 176, 967, 968, 971, 972, 974, 975, 1033-1038, 1254, 1255, and 1258-1287. Examples of sugar motifs for such a modified oligonucleotide or first modified oligonucleotide of an oligomeric compound or oligomeric duplex provided herein include, but are not limited to, (from 5′ to 3′): efyyyyyyyyyyyfyyyyyyyee, efyyyyyyyyyyyfyyyyyyyyy, efyyyyyyyyyyyfyyyyyyye, efyyyyyyyyyyyfyyyyyyy, e[FHNA]yyyyyyyyyyy[FHNA]yyyyyyyee, e[FHNA]yyyyyyyyyyy[FHNA]yyyyyyyyy, e[FHNA]yyyyyyyyyyy[FHNA]yyyyyyye, and e[FHNA]yyyyyyyyyyy[FHNA]yyyyyyy; wherein each “y” represents a 2′-OMe sugar moiety, each “e” represents a 2′-MOE sugar moiety, each “f” represents a 2′-fluoro sugar moiety, and each “[FHNA]” represents a 3′-fluoro-hexitol sugar moiety. In some embodiments, an oligomeric compound or oligomeric duplex provided herein comprises a modified oligonucleotide or first modified oligonucleotide (e.g., antisense oligonucleotide) comprising or consisting of a sequence of linked nucleosides having reduced fluorine content, such as, for example, any such sugar motifs set out herein, and having an internucleoside linkage motif containing one or more modified internucleoside linkages. In some such embodiments, the modified oligonucleotide or first modified oligonucleotide (e.g., antisense oligonucleotide) contains one or more phosphorothioate internucleoside linkages, such as, for example, 1, 2, 3, 4, 5 or 6 phosphorothioate internucleoside linkages. In some such embodiments, all the internucleoside linkages that are not phosphorothioate internucleoside linkages are phosphodiester internucleoside linkages. In some such embodiments, the number of nucleosides in the modified oligonucleotide or first modified oligonucleotide (e.g., antisense oligonucleotide) is 23, 22 or 21. Examples of internucleoside linkage motifs for a first modified oligonucleotide of an oligomeric compound provided herein include, but are not limited to, (from 5′ to 3′): ssooooooooooooooooooss, ssooosooooooooooooooss, ssoososoooooooooooooss, ssoooooooooooooooooos, ssoooooooooooooooooo, and ssooooooooooooooooss.

In certain embodiments, a modified oligonucleotide or first modified oligonucleotide (e.g., antisense oligonucleotide) of an oligomeric compound or oligomeric duplex having a reduced fluorine content provided herein contains one or more 2′-deoxynucleosides. In some embodiments, the one or more 2′-deoxynucleosides is/are in a region of the sequence of the modified oligonucleotide or first modified oligonucleotide (e.g., antisense oligonucleotide) that is between and includes the fifth and sixteenth, or the fifth and seventh, nucleosides counting from the 5′ terminus of the modified oligonucleotide or first modified oligonucleotide. In particular embodiments, the one or more 2′-deoxynucleosides is one or more of the fifth, sixth and/or seventh nucleoside(s) counting from the 5′ terminus of the modified oligonucleotide or first modified oligonucleotide (e.g., antisense oligonucleotide). In some embodiments, an oligomeric compound or oligomeric duplex having a reduced fluorine content provided herein has a modified oligonucleotide or first modified oligonucleotide (e.g., antisense oligonucleotide) containing no more than three 2′-deoxynucleosides. In particular embodiments, the modified oligonucleotide or first modified oligonucleotide (e.g., antisense oligonucleotide) contains no more than two 2′-deoxynucleosides. In some embodiments, the modified oligonucleotide or first modified oligonucleotide (e.g., antisense oligonucleotide) contains only one 2′-deoxynucleoside. In some embodiments, the modified oligonucleotide or first modified oligonucleotide (e.g., antisense oligonucleotide) does not contain any 2′-deoxynucleosides. In some embodiments, an oligomeric compound or oligomeric duplex having a reduced fluorine content provided herein has a modified oligonucleotide or first modified oligonucleotide (e.g., antisense oligonucleotide) containing one or more 2′-deoxynucleosides and one or more nucleosides containing a fluorine atom (e.g., a fluorine-containing modified sugar moiety e.g., a 2′-F sugar moiety). In some such embodiments, fewer than 20%, or fewer than 15%, of the nucleosides of the modified oligonucleotide or first modified oligonucleotide (e.g., antisense oligonucleotide) contain a fluorine atom. In particular embodiments, an oligomeric compound or oligomeric duplex having a reduced fluorine content provided herein has a modified oligonucleotide or first modified oligonucleotide (e.g., antisense oligonucleotide) containing one, two or three 2′-deoxynucleosides and one, two, or three nucleosides containing fluorine atom (e.g., a fluorine-containing modified sugar moiety e.g., a 2′-F sugar moiety). For example, in some embodiments, the modified oligonucleotide or first modified oligonucleotide (e.g., antisense oligonucleotide) contains one 2′-deoxynucleoside and three nucleosides containing a modified sugar moiety having a fluorine atom (e.g., a fluorine-containing modified sugar moiety e.g., a 2′-F sugar moiety). In some such embodiments, the number of nucleosides in the modified oligonucleotide or first modified oligonucleotide (e.g., antisense oligonucleotide) is 23, 22, or 21. In some such embodiments, the nucleobase sequence of the modified oligonucleotide or first modified oligonucleotide (e.g., antisense oligonucleotide) comprises or consists of a nucleobase sequence selected from among the sequences provided in Table A, Table B1 or SEQ ID NOS: 3-314, 627-782, 939-976, 1033-1038, 1254-1255, and 1258-1287. In some such embodiments, the nucleobase sequence of the modified oligonucleotide or first modified oligonucleotide (e.g., antisense oligonucleotide) comprises or consists of a nucleobase sequence selected from among the nucleobase sequences of SEQ ID NOS: 29,171,176, 967, 968, 971, 972, 974, 975, 1033-1038, 1254, 1255, and 1258-1287. Examples of sugar motifs for such a modified oligonucleotide or first modified oligonucleotide (e.g., antisense oligonucleotide) of an oligomeric compound or oligomeric duplex provided herein include, but are not limited to, (from 5′ to 3′): efyyydyyyyyyyfyfyyyyyee, efyyydyyyyyyyfyfyyyyyyy, efyyydyyyyyyyfyfyyyyye, efyyydyyyyyyyfyfyyyyy, efyyyfyyyyyyyfydyyyyyee, efyyyfyyyyyyyfydyyyyyyy, efyyyfyyyyyyyfydyyyyye, and efyyyfyyyyyyyfydyyyyy; wherein each “y” represents a 2′-OMe sugar moiety, each “e” represents a 2′-MOE sugar moiety, each “f” represents a 2′-fluoro sugar moiety and each “d” represents a 2′-deoxy sugar moiety. In some embodiments, the modified oligonucleotide or first modified oligonucleotide (e.g., antisense oligonucleotide) contains two 2′-deoxynucleosides and three nucleosides containing a modified sugar moiety having a fluorine atom (e.g., a fluorine-containing modified sugar moiety e.g., a 2′-F sugar moiety). In some such embodiments, the number of nucleosides in the modified oligonucleotide or first modified oligonucleotide (e.g., antisense oligonucleotide) is 23, 22 or 21. In some such embodiments, the nucleobase sequence of the modified oligonucleotide or first modified oligonucleotide (e.g., antisense oligonucleotide) comprises or consists of a nucleobase sequence selected from among the sequences provided in Table A, Table B1 or SEQ ID NOS: 3-314, 627-782, 939-976, 1033-1038, 1254-1255, and 1258-1287. In some such embodiments, the nucleobase sequence of the modified oligonucleotide or first modified oligonucleotide (e.g., antisense oligonucleotide) comprises or consists of a nucleobase sequence selected from among the nucleobase sequences of SEQ ID NOS: 29,171,176, 967, 968, 971, 972, 974, 975, 1033-1038, 1254, 1255, and 1258-1287. Examples of sugar motifs for such a modified oligonucleotide or first modified oligonucleotide (e.g., antisense oligonucleotide) of an oligomeric compound or oligomeric duplex provided herein include, but are not limited to, (from 5′ to 3′): efyydydyyyyyyfyfyyyyyee, efyydydyyyyyyfyfyyyyyyy, efyydydyyyyyyfyfyyyyye, and efyydydyyyyyyfyfyyyyy, wherein each “y” represents a 2′-OMe sugar moiety, each “e” represents a 2′-MOE sugar moiety, each “f” represents a 2′-fluoro sugar moiety and each “d” represents a 2′-deoxy sugar moiety. In some embodiments, the modified oligonucleotide or first modified oligonucleotide (e.g., antisense oligonucleotide) contains three 2′-deoxynucleosides and one nucleoside containing a modified sugar moiety having a fluorine atom (e.g., a fluorine-containing modified sugar moiety e.g., a 2′-F sugar moiety). In some such embodiments, the number of nucleosides in the modified oligonucleotide or first modified oligonucleotide (e.g., antisense oligonucleotide) is 23, 22, or 21. In some such embodiments, the nucleobase sequence of the modified oligonucleotide or first modified oligonucleotide (e.g., antisense oligonucleotide) comprises or consists of a nucleobase sequence selected from among the sequences provided in Table A, Table B1 or SEQ ID NOS: 3-314, 627-782, 939-976, 1033-1038, 1254-1255, 1258-1287. In some such embodiments, the nucleobase sequence of the modified oligonucleotide or first modified oligonucleotide (e.g., antisense oligonucleotide) comprises or consists of a nucleobase sequence selected from among the nucleobase sequences of SEQ ID NOS: 29,171,176, 967, 968, 971, 972, 974, 975, 1033-1038, 1254, 1255, and 1258-1287. Examples of sugar motifs for such a modified oligonucleotide or first modified oligonucleotide (e.g., antisense oligonucleotide) of an oligomeric compound or oligomeric duplex provided herein include, but are not limited to, (from 5′ to 3′): efyyydyyyyyyydydyyyyyee, efyyydyyyyyyydydyyyyyyy, efyyydyyyyyyydydyyyyye, and efyyydyyyyyyydydyyyyy, wherein each “y” represents a 2′-OMe sugar moiety, each “e” represents a 2′-MOE sugar moiety, each “f” represents a 2′-fluoro sugar moiety and each “d” represents a 2′-deoxy sugar moiety. In some such embodiments, the modified oligonucleotide or first modified oligonucleotide (e.g., antisense oligonucleotide) contains one or more phosphorothioate internucleoside linkages, such as, for example, 1, 2, 3, 4, 5 or 6 phosphorothioate internucleoside linkages. In some embodiments, an oligomeric compound or oligomeric duplex provided herein comprises a modified oligonucleotide or first modified oligonucleotide (e.g., antisense oligonucleotide) comprising or consisting of a contiguous sequence of linked nucleosides having reduced fluorine content, such as, for example, any such sugar motifs set out herein, and having an internucleoside linkage motif comprising one or more modified internucleoside linkages. In some embodiments, the modified oligonucleotide or first modified oligonucleotide (e.g., antisense oligonucleotide) contains one or more phosphorothioate internucleoside linkages. In some such embodiments, all the internucleoside linkages that are not phosphorothioate internucleoside linkages are phosphodiester linkages. In some such embodiments, the number of nucleosides in the modified oligonucleotide or first modified oligonucleotide (e.g., antisense oligonucleotide) is 23, 22 or 21. Examples of internucleoside linkage motifs for a modified oligonucleotide or first modified oligonucleotide (e.g., antisense oligonucleotide) of an oligomeric compound or oligomeric duplex provided herein include, but are not limited to, (from 5′ to 3′): ssooooooooooooooooooss, ssooosooooooooooooooss, ssoososoooooooooooooss, ssoooooooooooooooooos, ssoooooooooooooooooo, and ssooooooooooooooooss, wherein each “o” represents a phosphodiester linkage and each “s” represents a phosphorothioate linkage.

In some embodiments, an oligomeric compound having a reduced fluorine content provided herein contains a modified oligonucleotide having reduced fluorine content (e.g., fewer than 40% or fewer than 35% of the nucleosides of the modified oligonucleotide comprises a fluorine atom) which comprises or consists of a region having a nucleobase sequence complementary to a sequence that is complementary to a sequence in a PLN nucleic acid. In some embodiments, an oligomeric duplex having a reduced fluorine content provided herein contains a second modified oligonucleotide (e.g., sense oligonucleotide) having reduced fluorine content (e.g., fewer than 40% of the nucleosides of the modified oligonucleotide or second modified oligonucleotide comprising a fluorine atom) which comprises or consists of a region having a nucleobase sequence complementary to a first oligonucleotide (e.g., antisense oligonucleotide) comprising or consisting of a region having a nucleobase sequence complementary to a sequence in a PLN nucleic acid. In some such embodiments, fewer than 30%, fewer than 25%, fewer than 20%, fewer than 15%, fewer than 14%, fewer than 13%, fewer than 12%, fewer than 10%, fewer than 5%, or fewer than 1% of the nucleosides in the modified oligonucleotide or second modified oligonucleotide (e.g., sense oligonucleotide) contain a fluorine atom, e.g., a 2′-F sugar moiety. In particular embodiments, no more than 3, or no more than 2, nucleosides in the modified oligonucleotide or second modified oligonucleotide (e.g., sense oligonucleotide) contain a fluorine atom (e.g., a fluorine-containing modified sugar moiety e.g., a 2′-F sugar moiety). In some embodiments, only one or no nucleosides in the modified oligonucleotide or second modified oligonucleotide (e.g., sense oligonucleotide) contain a fluorine atom (e.g., a fluorine-containing modified sugar moiety e.g., a 2′-F sugar moiety). In certain embodiments, none of the nucleosides before the seventh or after the eleventh nucleoside counting from the 5′ terminus of the modified oligonucleotide or second modified oligonucleotide (e.g., sense oligonucleotide) contain a fluorine atom (e.g., a fluorine-containing modified sugar moiety e.g., a 2′-F sugar moiety). In certain embodiments, none of the nucleosides before the tenth or after the eleventh nucleoside counting from the 5′ terminus of the modified oligonucleotide or second modified oligonucleotide (e.g., sense oligonucleotide) contain a fluorine atom (e.g., a fluorine-containing modified sugar moiety e.g., a 2′-F sugar moiety). In certain embodiments, an oligomeric duplex containing a second modified oligonucleotide (e.g., sense oligonucleotide) that has a sequence of linked nucleosides having reduced fluorine content provided herein contains a first modified oligonucleotide (e.g., antisense oligonucleotide) having a reduced fluorine content. In certain such embodiments, the first modified oligonucleotide (e.g., antisense oligonucleotide) of such an oligomeric duplex is a first modified oligonucleotide having reduced fluorine content as described herein, including, for example, a first modified oligonucleotide having a sugar motif as described herein. In some embodiments, the nucleobase sequence of the first modified oligonucleotide (e.g., antisense oligonucleotide) comprises or consists of a nucleobase sequence selected from among the sequences provided in Table A, Table B1 or SEQ ID NOS: 3-314, 627-782, 939-976, 1033-1038, 1254-1255, and 1258-1287. In some such embodiments, the nucleobase sequence of the first modified oligonucleotide (e.g., antisense oligonucleotide) comprises or consists of a nucleobase sequence selected from among the nucleobase sequences of SEQ ID NOS: 29,171,176, 967, 968, 971, 972, 974, 975, 1033-1038, 1254, 1255, and 1258-1287. In particular embodiments, the total fluorine content of the oligomeric compound or oligomeric duplex is reduced (e.g., fewer than 40% of the total nucleosides in the compound or duplex comprising a fluorine atom). In some embodiments, a modified oligonucleotide, which comprises or consists of a region having a nucleobase sequence complementary to a nucleobase sequence complementary to a sequence in a PLN nucleic acid, of an oligomeric compound provided herein, or a second modified oligonucleotide (e.g., sense oligonucleotide) of an oligomeric duplex provided herein, contains one or more nucleosides containing a fluorine atom (e.g., a fluorine-containing modified sugar moiety e.g., a 2′-F sugar moiety). In some embodiments, the modified oligonucleotide or second modified oligonucleotide (e.g., sense oligonucleotide) contains one, two, three or four nucleosides containing a fluorine atom (e.g., a fluorine-containing modified sugar moiety e.g., a 2′-F sugar moiety). In some embodiments, the number of nucleosides in the modified oligonucleotide or second modified oligonucleotide (e.g., sense oligonucleotide) is 21, 20 or 19. In particular embodiments, one or more, or two or more, of the seventh, ninth and eleventh nucleosides counting from the 5′ terminus of the modified oligonucleotide or second modified oligonucleotide (e.g., sense oligonucleotide) contains a fluorine atom (e.g., a fluorine-containing modified sugar moiety e.g., a 2′-F sugar moiety). In particular embodiments, the seventh, ninth and eleventh nucleosides counting from the 5′ terminus of the modified oligonucleotide or second modified oligonucleotide (e.g., sense oligonucleotide) contain a fluorine atom (e.g., a fluorine-containing modified sugar moiety e.g., a 2′-F sugar moiety). In some embodiments, the tenth and eleventh nucleosides counting from the 5′ terminus of the modified oligonucleotide or second modified oligonucleotide (e.g., sense oligonucleotide) contain a fluorine atom (e.g., a fluorine-containing modified sugar moiety e.g., a 2′-F sugar moiety). In some embodiments, the modified oligonucleotide of an oligomeric compound or the second modified oligonucleotide (e.g., sense oligonucleotide) of an oligomeric duplex provided herein does not contain any fluorine atoms. In some such embodiments, one or more of the nucleosides contain a 2′-OMe sugar moiety or a 2′-MOE sugar moiety. In some embodiments, any of the nucleosides that do not contain a fluorine atom contain a 2′-OMe sugar moiety or a 2′-MOE sugar moiety. In some embodiments, at least 10%, at least 20%, at least 30%, at least 40%, at least 50%, or at least 60%, or at least 65%, or at least 70%, or at least 80%, or at least 85%, or at least 90% of the nucleosides of the modified oligonucleotide or second modified oligonucleotide contain a 2′-OMe sugar moiety. In particular embodiments, the one or more nucleosides comprising a 2′-OMe sugar moiety are in a region of the sequence of the modified oligonucleotide or second modified oligonucleotide (e.g., sense oligonucleotide) between and including the third and nineteenth nucleosides counting from the 5′ terminus of the modified oligonucleotide or second modified oligonucleotide. In some embodiments, the 3′-terminal nucleoside of the modified oligonucleotide or second modified oligonucleotide (e.g., sense oligonucleotide) contains a 2′-OMe sugar moiety and/or the nucleoside immediately 5′ of the 3′-terminal nucleoside of the modified oligonucleotide or second modified oligonucleotide contains a 2′-OMe sugar moiety. In some embodiments, the 5′-terminal nucleoside of the modified oligonucleotide or second modified oligonucleotide (e.g., sense oligonucleotide) comprises a 2′-OMe sugar moiety and/or the nucleoside immediately 3′ of the 5′-terminal nucleoside of the modified oligonucleotide or second modified oligonucleotide comprises a 2′-OMe sugar moiety. In some embodiments, all of the nucleosides contain a 2′-OMe sugar moiety. In some embodiments, one or more of the nucleosides of the modified oligonucleotide or second modified oligonucleotide (e.g., sense oligonucleotide) contain a 2′-MOE sugar moiety. In certain embodiments, the 3′-terminal nucleoside of the modified oligonucleotide or second modified oligonucleotide (e.g., sense oligonucleotide) contains a 2′-MOE sugar moiety and/or the nucleoside immediately 5′ of the 3′-terminal nucleoside of the modified oligonucleotide or second modified oligonucleotide contains a 2′-MOE sugar moiety. In certain embodiments, the 5′-terminal nucleoside of the modified oligonucleotide or second modified oligonucleotide (e.g., sense oligonucleotide) contains a 2′-MOE sugar moiety and/or the nucleoside immediately 3′ of the 5′-terminal nucleoside of the modified oligonucleotide or second modified oligonucleotide contains a 2′-MOE sugar moiety. In particular embodiments, the 5′-terminal nucleoside, the nucleoside immediately 3′ of the 5′-terminal nucleoside, the 3′-terminal nucleoside, and the nucleoside immediately 5′ of the 3′-terminal nucleoside of the modified oligonucleotide or second modified oligonucleotide (e.g., sense oligonucleotide) contains a 2′-MOE sugar moiety.

In some embodiments, an oligomeric compound having reduced fluorine content provided herein contains a modified oligonucleotide comprising or consisting of a region having a nucleobase sequence complementary to a nucleobase sequence that is complementary to a sequence in a PLN nucleic acid wherein the modified oligonucleotide contains no more than four nucleosides containing a fluorine atom (e.g., a fluorine-containing modified sugar moiety, e.g., a 2′-fluoro sugar moiety). In some embodiments, an oligomeric duplex having reduced fluorine content provided herein contains a second modified oligonucleotide (e.g., a sense oligonucleotide) containing no more than four nucleosides containing a fluorine atom (e.g., a fluorine-containing modified sugar moiety e.g., a 2′-F sugar moiety). For example, in some embodiments, the modified oligonucleotide or second modified oligonucleotide (e.g., a sense oligonucleotide) contains four nucleosides containing a modified sugar moiety having a fluorine atom. In some such embodiments, the number of nucleosides in the modified oligonucleotide or second modified oligonucleotide is 21, 20 or 19. In some such embodiments, the nucleobase sequence of the modified oligonucleotide or second modified oligonucleotide (e.g., a sense oligonucleotide) comprises or consists of a nucleobase sequence selected from among the sequences provided in Table A, Table B2 or SEQ ID NOS: 315-626, 783-938, 977-1016, 1027-1032, 1256-1257, and 1288-1290. In some such embodiments, the nucleobase sequence of the modified oligonucleotide or second modified oligonucleotide (e.g., a sense oligonucleotide) comprises or consists of a nucleobase sequence selected from among the nucleobase sequences of SEQ ID NOS: 341, 483, 488, 1011-1016, 1027-1032, 1256-1257, and 1288-1290. Examples of sugar motifs for such a modified oligonucleotide or second modified oligonucleotide (e.g., a sense oligonucleotide) of an oligomeric compound or oligomeric duplex provided herein include, but are not limited to, (from 5′ to 3′): eeyyyyfyfffyyyyyyyyee, eeyyyyfyfffyyyyyyyyyy, yyyyyyfyfffyyyyyyyyee, yyyyyyfyfffyyyyyyyyyy, eeyyyyfyfffyyyyyyee, eeyyyyfyfffyyyyyyyy, yyyyyyfyfffyyyyyyee, yyyyyyfyfffyyyyyyyy, eeyyfyfffyyyyyyyyee, eeyyfyfffyyyyyyyyyy, yyyyfyfffyyyyyyyyee, and yyyyfyfffyyyyyyyyyy, wherein each “y” represents a 2′-OMe sugar moiety, each “e” represents a 2′-MOE sugar moiety, and each “f” represents a 2′-fluoro sugar. In some embodiments, an oligomeric compound or oligomeric duplex provided herein has a modified oligonucleotide or second modified oligonucleotide (e.g., sense oligonucleotide) containing no more than three nucleosides containing a fluorine atom (e.g., a fluorine-containing modified sugar moiety). For example, in some embodiments, the modified oligonucleotide or second modified oligonucleotide (e.g., a sense oligonucleotide) contains three nucleosides containing a modified sugar moiety having a fluorine atom. In some such embodiments, the number of nucleosides in the modified oligonucleotide or second modified oligonucleotide is independently 21, 20 or 19. In some such embodiments, the nucleobase sequence of the modified oligonucleotide or second modified oligonucleotide (e.g., a sense oligonucleotide) comprises or consists of a nucleobase sequence selected from among the sequences provided in Table A, Table B2 or SEQ ID NOS: 315-626, 783-938, 977-1016, 1027-1032, 1256-1257, and 1288-1290. In some such embodiments, the nucleobase sequence of the modified oligonucleotide or second modified oligonucleotide (e.g., a sense oligonucleotide) comprises or consists of a nucleobase sequence selected from among the nucleobase sequences of SEQ ID NOS: 341, 483, 488, 1011-1016, 1027-1032, 1256-1257, and 1288-1290. Examples of sugar motifs for such a modified oligonucleotide or second modified oligonucleotide (e.g., a sense oligonucleotide) of an oligomeric compound or oligomeric duplex provided herein include, but are not limited to, (from 5′ to 3′): eeyyyyfyfyfyyyyyyyyee, eeyyyyfyfyfyyyyyyyyyy, yyyyyyfyfyfyyyyyyyyee, and yyyyyyfyfyfyyyyyyyyyy, wherein each “y” represents a 2′-OMe sugar moiety, each “e” represents a 2′-MOE sugar moiety, and each “f” represents a 2′-fluoro sugar moiety. In some embodiments, an oligomeric compound or oligomeric duplex provided herein has a modified oligonucleotide or second modified oligonucleotide (e.g., a sense oligonucleotide) containing no more than two nucleosides containing a fluorine atom (e.g., a fluorine-containing modified sugar moiety e.g., a 2′-F sugar moiety). For example, in some embodiments, the modified oligonucleotide or second modified oligonucleotide (e.g., a sense oligonucleotide) contains two nucleosides containing a modified sugar moiety having a fluorine atom. In some such embodiments, the number of nucleosides in the second modified oligonucleotide is 21, 20, or 19. In some such embodiments, the nucleobase sequence of the modified oligonucleotide or second modified oligonucleotide (e.g., a sense oligonucleotide) comprises or consists of a nucleobase sequence selected from among the sequences provided in Table A, Table B2 or SEQ ID NOS: 315-626, 783-938, 977-1016, 1027-1032, 1256-1257, and 1288-1290. In some such embodiments, the nucleobase sequence of the modified oligonucleotide or second modified oligonucleotide (e.g., a sense oligonucleotide) comprises or consists of a nucleobase sequence selected from among the nucleobase sequences of SEQ ID NOS: 341, 483, 488, 1011-1016, 1027-1032, 1256-1257, and 1288-1290. Examples of sugar motifs for such a modified oligonucleotide or second modified oligonucleotide of an oligomeric compound or oligomeric duplex provided herein include, but are not limited to, (from 5′ to 3′): eeyyyyyyyffyyyyyyyyee, eeyyyyyyyffyyyyyyyyyy, yyyyyyyyyffyyyyyyyyee, yyyyyyyyyffyyyyyyyyyy, eeyyyyyffyyyyyyyyee, eeyyyyyffyyyyyyyyyy, yyyyyyyffyyyyyyyyee, and yyyyyyyffyyyyyyyyyy, wherein each “y” represents a 2′-OMe sugar moiety, each “e” represents a 2′-MOE sugar moiety, and each “f” represents a 2′-fluoro sugar moiety. In some embodiments, the modified oligonucleotide or second modified oligonucleotide (e.g., a sense oligonucleotide) of an oligomeric compound or oligomeric duplex provided herein does not contain any fluorine atoms. In some such embodiments, the modified oligonucleotide or second modified oligonucleotide may contain one or more 2′-deoxynucleosides. In particular such embodiments, the modified oligonucleotide or second modified oligonucleotide (e.g., a sense oligonucleotide) contains only one 2′-deoxynucleoside. In some such embodiments, the number of nucleosides in the modified oligonucleotide or second modified oligonucleotide is 21, 20, or 19. In some such embodiments, the nucleobase sequence of the modified oligonucleotide or second modified oligonucleotide (e.g., a sense oligonucleotide) comprises or consists of a nucleobase sequence selected from among the sequences provided in Table A, Table B2 or SEQ ID NOS: 315-626, 783-938, 977-1016, 1027-1032, 1256-1257, and 1288-1290. In some such embodiments, the nucleobase sequence of the modified oligonucleotide or second modified oligonucleotide (e.g., a sense oligonucleotide) comprises or consists of a nucleobase sequence selected from among the nucleobase sequences of SEQ ID NOS: 341, 483, 488, 1011-1016, 1027-1032, 1256-1257, and 1288-1290. Examples of a sugar motif for such a modified oligonucleotide or second modified oligonucleotide (e.g., a sense oligonucleotide) of an oligomeric compound or oligomeric duplex provided herein include, but are not limited to, (from 5′ to 3′): eeyyyyyyyydyyyyyyyyee, eeyyyyyyyydyyyyyyyyyy, yyyyyyyyyydyyyyyyyyee, and yyyyyyyyyydyyyyyyyyyy, wherein each “y” represents a 2′-OMe sugar moiety, each “e” represents a 2′-MOE sugar moiety, and each “d” represents a 2′-deoxy sugar moiety. In some embodiments, all of the nucleosides of the modified oligonucleotide or second modified oligonucleotide (e.g., a sense oligonucleotide) contain a 2′-modified sugar moiety and none of the nucleosides contain a fluorine atom. In particular such embodiments, none of the modified sugar moieties is a 2′-deoxynucleoside. In some embodiments, the number of nucleosides in the modified oligonucleotide or second modified oligonucleotide is 21, 20, or 19. In some such embodiments, the nucleobase sequence of the modified oligonucleotide or second modified (e.g., a sense oligonucleotide) oligonucleotide comprises or consists of a nucleobase sequence selected from among the sequences provided in Table A, Table B2 or SEQ ID NOS: 315-626, 783-938, 977-1016, 1027-1032, 1256-1257, and 1288-1290. In some such embodiments, the nucleobase sequence of the modified oligonucleotide or second modified oligonucleotide (e.g., a sense oligonucleotide) comprises or consists of a nucleobase sequence selected from among the nucleobase sequences of SEQ ID NOS: 341, 483, 488, 1011-1016, 1027-1032, 1256-1257, and 1288-1290. Examples of a sugar motif for a modified oligonucleotide or second modified oligonucleotide (e.g., a sense oligonucleotide) of an oligomeric compound or oligomeric duplex provided herein that does contain a fluorine atom include, but are not limited to, (from 5′ to 3′): eeyyyyyyyyyyyyyyyyyee, eeyyyyyyyyyyyyyyyyyyy, yyyyyyyyyyyyyyyyyyyee, and yyyyyyyyyyyyyyyyyyyyy, wherein each “y” represents a 2′-OMe sugar moiety and each “e” represents a 2′-MOE sugar moiety. In some embodiments, an oligomeric compound or oligomeric duplex provided herein comprises a modified oligonucleotide or second modified oligonucleotide (e.g., a sense oligonucleotide) containing a sequence of linked nucleosides having reduced fluorine content, such as, for example, any such sugar motifs set out herein, and having an internucleoside linkage motif comprising one or more modified internucleoside linkages. In such embodiments, the modified oligonucleotide or second modified oligonucleotide (e.g., a sense oligonucleotide) may contain one or more mesyl phosphoramidate internucleoside linkages and/or one or more phosphorothioate internucleoside linkages, such as, for example, 1, 2, 3, 4, 5, or 6 phosphorothioate internucleoside linkages. In some embodiments, no more than 4 of the internucleoside linkages of the modified oligonucleotide or second modified oligonucleotide (e.g., a sense oligonucleotide) are phosphorothioate internucleoside linkages. In some such embodiments, all the internucleoside linkages that are not phosphorothioate internucleoside linkages are phosphodiester internucleoside linkages. Examples of internucleoside linkage motifs for a modified oligonucleotide or second modified oligonucleotide (e.g., a sense oligonucleotide) of an oligomeric compound or oligomeric duplex provided herein include, but are not limited to, (from 5′ to 3′): ssooooooooooooooooss, ssooooooosooooooooss, ssooooooooooooooss, and ssoooooozozoooooooss, wherein each “o” represents a phosphodiester linkage, each “z” represents a mesyl phosphoramidate linkage, and each “s” represents a phosphorothioate linkage.

In some embodiments, oligomeric duplexes having reduced fluorine content provided herein contain a first modified oligonucleotide (e.g., an antisense oligonucleotide) having a reduced fluorine content and a second modified oligonucleotide (e.g., a sense oligonucleotide) having a reduced fluorine content. In some such embodiments, the number of nucleosides in the first modified oligonucleotide is 23, 22, or 21 and the number of nucleosides in the second modified oligonucleotide is 21, 20, or 19. In some such embodiments, the number of nucleosides in the first modified oligonucleotide is 23 and the number of nucleosides in the second modified oligonucleotide is 21. In some embodiments, the number of nucleosides in the first modified oligonucleotide is 21 and the number of nucleosides in the second modified oligonucleotide is 19. In some embodiments, the nucleobase sequence of the first modified oligonucleotide (e.g., an antisense oligonucleotide) comprises or consists of a nucleobase sequence selected from among the sequences provided in Table A, Table B1 or SEQ ID NOS: 3-314, 627-782, 939-976, 1033-1038, 1254-1255, and 1258-1287. In some embodiments, the nucleobase sequence of the first modified oligonucleotide comprises or consists of a nucleobase sequence selected from among the nucleobase sequences of SEQ ID NOS: 29,171,176, 967, 968, 971, 972, 974, 975, 1033-1038, 1254, 1255, and 1258-1287. In some such embodiments, the nucleobase sequence of the second modified oligonucleotide (e.g., a sense oligonucleotide) comprises or consists of a nucleobase sequence complementary to a sequence in the nucleobase sequence of the first modified oligonucleotide, or of a nucleobase sequence selected from among the sequences provided in Table A, Table B2 or SEQ ID NOS: 315-626, 783-938, 977-1016, 1027-1032, 1256-1257, and 1288-1290. In some embodiments, an oligomeric duplex having a reduced fluorine content provided herein has a first modified oligonucleotide (e.g., antisense oligonucleotide) having a reduced fluorine content containing one or more nucleosides containing a fluorine atom and/or a second modified oligonucleotide (e.g., a sense oligonucleotide) having a reduced fluorine content containing one or more nucleosides containing a fluorine atom (e.g., a fluorine-containing modified sugar moiety e.g., a 2′-F sugar moiety). In some such embodiments, the first modified oligonucleotide and the second modified oligonucleotide each independently contain none or one, two, three, or four nucleosides containing a fluorine atom. In some such embodiments, the first and/or second modified oligonucleotide contains no 2′-deoxynucleosides or contains one or more 2′-deoxynucleosides. In some such embodiments, the first modified oligonucleotide (e.g., an antisense oligonucleotide) contains one or more 2′-deoxynucleosides and one or more nucleosides containing a fluorine atom (e.g., a fluorine-containing modified sugar moiety e.g., a 2′-F sugar moiety). In particular embodiments, the first modified oligonucleotide (e.g., an antisense oligonucleotide) contains one, two or three 2′-deoxynucleosides and one, two, or three nucleosides containing fluorine atom (e.g., a fluorine-containing modified sugar moiety, e.g., a 2′-F sugar moiety). For example, in some embodiments, the first modified oligonucleotide (e.g., an antisense oligonucleotide) contains only one 2′-deoxynucleoside and three nucleosides containing a sugar moiety having a fluorine atom (e.g., a fluorine-containing modified sugar moiety e.g., a 2′-F sugar moiety). In some embodiments, the first modified oligonucleotide (e.g., an antisense oligonucleotide) contains no more than three 2′-deoxynucleosides or no more than two 2′-deoxynucleosides. In some such embodiments, the first modified oligonucleotide or the second modified nucleotide contains only one 2′-deoxynucleoside. In some embodiments, all of the nucleosides of the first modified oligonucleotide and of the second modified oligonucleotide contain a 2′-modified sugar moiety and one or more of the nucleosides contain a fluorine atom (e.g., a fluorine-containing modified sugar moiety e.g., a 2′-F sugar moiety). In some such embodiments, one or more of the nucleosides contain a 2′-OMe sugar moiety or a 2′-MOE sugar moiety. In some embodiments, any of the nucleosides in the first modified oligonucleotide or the second modified oligonucleotide that do not contain a fluorine atom contain a 2′-OMe sugar moiety or a 2′-MOE sugar moiety.

In some embodiments, oligomeric duplexes having reduced fluorine content provided herein contain a first modified oligonucleotide (e.g., an antisense oligonucleotide) having a reduced fluorine content and a second modified oligonucleotide (e.g., a sense oligonucleotide) having a reduced fluorine content and each of the first modified oligonucleotide and the second modified oligonucleotide contain no more than four nucleosides containing a fluorine atom. For example, in some such embodiments, the first modified oligonucleotide contains four nucleosides containing a fluorine atom and the second modified oligonucleotide contains four, three, two, one, or no nucleosides containing a fluorine atom, or the first modified oligonucleotide contains four, three, two, one, or no nucleosides containing a fluorine atom, and the second modified oligonucleotide contains four nucleosides containing a fluorine atom. In some such embodiments, the number of nucleosides in the first modified oligonucleotide is 23, 22, or 21 and the number of nucleosides in the second modified oligonucleotide is 21, 20, or 19. In some such embodiments, the number of nucleosides in the first modified oligonucleotide is 23 and the number of nucleosides in the second modified oligonucleotide is 21. In some embodiments, the number of nucleosides in the first modified oligonucleotide is 21 and the number of nucleosides in the second modified oligonucleotide is 19. In some embodiments, the nucleobase sequence of the first modified oligonucleotide (e.g., an antisense oligonucleotide) comprises or consists of a nucleobase sequence selected from among the sequences provided in Table A, Table B1 or SEQ ID NOS: 3-314, 627-782, 939-976, 1033-1038, 1254-1255, and 1258-1287. In some embodiments, the nucleobase sequence of the first modified oligonucleotide (e.g., an antisense oligonucleotide) comprises or consists of a nucleobase sequence selected from among the nucleobase sequences of SEQ ID NOS: 29, 171, 176, 967, 968, 971, 972, 974, 975, 1033-1038, 1254, 1255, and 1258-1287. In some such embodiments, the nucleobase sequence of the second modified oligonucleotide (e.g., a sense oligonucleotide) comprises or consists of a nucleobase sequence complementary to a sequence in the nucleobase sequence of the first modified oligonucleotide, or of a nucleobase sequence selected from among the sequences provided in Table A, Table B2 or SEQ ID NOS: 315-626, 783-938, 977-1016, 1027-1032, 1256-1257, and 1288-1290. In some such embodiments, the nucleobase sequence of the second modified oligonucleotide (e.g., a sense oligonucleotide) comprises or consists of a nucleobase sequence selected from among the nucleobase sequences of SEQ ID NOS: 341, 483, 488, 1011-1016, 1027-1032, 1256-1257, and 1288-1290. In some embodiments, each of the first modified oligonucleotide and the second modified oligonucleotide contains no more than three nucleosides containing a fluorine atom. For example, in some such embodiments, the first modified oligonucleotide contains three nucleosides containing a fluorine atom and the second modified oligonucleotide contains three, two, one, or no nucleosides containing a fluorine atom, or the first modified oligonucleotide contains three, two, one, or no nucleosides containing a fluorine atom, and the second modified oligonucleotide contains three nucleosides containing a fluorine atom. In particular such embodiments, the first modified oligonucleotide has three nucleosides comprising a fluorine and the second modified oligonucleotide has two or three nucleosides comprising a fluorine. In some such embodiments, the number of nucleosides in the first modified oligonucleotide is 23, 22, or 21 and the number of nucleosides in the second modified oligonucleotide is 21, 20 or 19. In some such embodiments, the number of nucleosides in the first modified oligonucleotide is 23 and the number of nucleosides in the second modified oligonucleotide is 21. In some embodiments, the number of nucleosides in the first modified oligonucleotide is 21 and the number of nucleosides in the second modified oligonucleotide is 19. In some embodiments, the nucleobase sequence of the first modified oligonucleotide (e.g., an antisense oligonucleotide) comprises or consists of a nucleobase sequence selected from among the sequences provided in Table A, Table B1 or SEQ ID NOS: 3-314, 627-782, 939-976, 1033-1038, 1254-1255, and 1258-1287. In some embodiments, the nucleobase sequence of the first modified oligonucleotide (e.g., an antisense oligonucleotide) comprises or consists of a nucleobase sequence selected from among the nucleobase sequences of SEQ ID NOS: 29, 171, 176, 967, 968, 971, 972, 974, 975, 1033-1038, 1254, 1255, and 1258-1287. In some such embodiments, the nucleobase sequence of the second modified oligonucleotide (e.g., a sense oligonucleotide) comprises or consists of a nucleobase sequence complementary to a sequence in the nucleobase sequence of the first modified oligonucleotide, or of a nucleobase sequence selected from among the sequences provided in Table A, Table B2 or SEQ ID NOS: 315-626, 783-938, 977-1016, 1027-1032, 1256-1257, and 1288-1290. In some such embodiments, the nucleobase sequence of the second modified oligonucleotide (e.g., a sense oligonucleotide) comprises or consists of a nucleobase sequence selected from among the nucleobase sequences of SEQ ID NOS: 341, 483, 488, 1011-1016, 1027-1032, 1256-1257, and 1288-1290. In some embodiments, each of the first modified oligonucleotide and the second modified oligonucleotide contains no more than two nucleosides containing a fluorine atom. For example, in some such embodiments, the first modified oligonucleotide (e.g., an antisense oligonucleotide) contains two nucleosides containing a fluorine atom and the second modified oligonucleotide (e.g., a sense oligonucleotide) contains two, one, or no nucleosides containing a fluorine atom, or the first modified oligonucleotide contains two, one, or no nucleosides containing a fluorine atom, and the second modified oligonucleotide contains two nucleosides containing a fluorine atom. In some such embodiments, the number of nucleosides in the first modified oligonucleotide is 23, 22, or 21 and the number of nucleosides in the second modified oligonucleotide is 21, 20 or 19. In some such embodiments, the number of nucleosides in the first modified oligonucleotide is 23 and the number of nucleosides in the second modified oligonucleotide is 21. In some embodiments, the number of nucleosides in the first modified oligonucleotide is 21 and the number of nucleosides in the second modified oligonucleotide is 19. In some embodiments, the nucleobase sequence of the first modified oligonucleotide (e.g., an antisense oligonucleotide) comprises or consists of a nucleobase sequence selected from among the sequences provided in Table A, Table B1 or SEQ ID NOS: 3-314, 627-782, 939-976, and 1033-1038, 1254-1255, and 1258-1287. In some embodiments, the nucleobase sequence of the first modified oligonucleotide (e.g., an antisense oligonucleotide) comprises or consists of a nucleobase sequence selected from among the nucleobase sequences of SEQ ID NOS: 29,171,176, 967, 968, 971, 972, 974, 975, 1033-1038, 1254, 1255, and 1258-1287. In some such embodiments, the nucleobase sequence of the second modified oligonucleotide (e.g., a sense oligonucleotide) comprises or consists of a nucleobase sequence complementary to a sequence in the nucleobase sequence of the first modified oligonucleotide, or of a nucleobase sequence selected from among the sequences provided in Table A, Table B2 or SEQ ID NOS: 315-626, 783-938, 977-1016, 1027-1032, 1256-1257, and 1288-1290. In some such embodiments, the nucleobase sequence of the second modified oligonucleotide (e.g., a sense oligonucleotide) comprises or consists of a nucleobase sequence selected from among the nucleobase sequences of SEQ ID NOS: 341, 483, 488, 1011-1016, 1027-1032, 1256-1257, and 1288-1290.

In some embodiments, an oligomeric duplex having a reduced fluorine content provided herein contains a first modified oligonucleotide (e.g., an antisense oligonucleotide) having a reduced fluorine content having a first sugar motif (1^{st}) and a second modified oligonucleotide (e.g., a sense oligonucleotide) having a reduced fluorine content having a second sugar motif (2^{nd}) and the first and second sugar motifs are independently selected from among the first and second sugar motifs provided in Table C. In some embodiments, an oligomeric duplex having a reduced fluorine content provided herein comprises a first modified oligonucleotide (e.g., an antisense oligonucleotide) having a reduced fluorine content having a first sugar motif (1^{st}) and a second modified oligonucleotide (e.g., a sense oligonucleotide) having a reduced fluorine content having a second sugar motif (2^{nd}) and the first and second sugar motifs are any one of the exemplary combinations of first and second sugar motifs set out in Table C. In Table C, each “y” represents a 2′-OMe sugar moiety, each “e” represents a 2′-MOE sugar moiety, each “f” represents a 2′-fluoro sugar moiety, each “[FfHNA]” represents a 3′-fluoro-hexitol sugar moiety, and each “d” represents a 2′-deoxy sugar moiety.

TABLE C
Exemplary Modified Oligonucleotide Sugar Motifs

| Exemplary | | |
|---|---|---|
| Combination | | |
| Number | First Sugar Motif | Second Sugar Motif |
| 1 | efyydydyyyyyyfyfyyyyyee | eeyyyyfyfyfyyyyyyyyee |
| 2 | efyydydyyyyyyfyfyyyyyyy | yyyyyyfyfyfyyyyyyyyyy |
| 3 | efyydydyyyyyyfyfyyyyyee | yyyyyyfyfyfyyyyyyyyyy |
| 4 | efyydydyyyyyyfyfyyyyyyy | eeyyyyfyfyfyyyyyyyyee |
| 5 | efyyydyyyyyyyfyfyyyyyee | eeyyyyyyyffyyyyyyyyee |
| 6 | efyyydyyyyyyyfyfyyyyyyy | yyyyyyyyyffyyyyyyyyyy |
| 7 | efyyydyyyyyyyfyfyyyyyee | yyyyyyyyyffyyyyyyyyyy |
| 8 | efyyydyyyyyyyfyfyyyyyyy | eeyyyyyyyffyyyyyyyyee |
| 9 | efyydydyyyyyyfyfyyyyyee | eeyyyyyyyffyyyyyyyyee |
| 10 | efyydydyyyyyyfyfyyyyyyy | yyyyyyyyyffyyyyyyyyyy |
| 11 | efyydydyyyyyyfyfyyyyyee | yyyyyyyyyffyyyyyyyyyy |
| 12 | efyydydyyyyyyfyfyyyyyyy | eeyyyyyyyffyyyyyyyyee |
| 13 | efyyydyyyyyyyfyfyyyyyee | eeyyyyfyfyfyyyyyyyyee |
| 14 | efyyydyyyyyyyfyfyyyyyyy | yyyyyyfyfyfyyyyyyyyyy |
| 15 | efyyydyyyyyyyfyfyyyyyee | yyyyyyfyfyfyyyyyyyyyy |
| 16 | efyyydyyyyyyyfyfyyyyyyy | eeyyyyfyfyfyyyyyyyyee |
| 17 | efyydydyyyyyyfyfyyyyyee | eeyyyyfyfffyyyyyyyyee |
| 18 | efyydydyyyyyyfyfyyyyyyy | yyyyyyfyfffyyyyyyyyyy |
| 19 | efyydydyyyyyyfyfyyyyyee | yyyyyyfyfffyyyyyyyyyy |
| 20 | efyydydyyyyyyfyfyyyyyyy | eeyyyyfyfffyyyyyyyyee |
| 21 | efyyyyyyyyyyyfyfyyyyyee | eeyyyyfyfffyyyyyyyyee |
| 22 | efyyyyyyyyyyyfyfyyyyyyy | yyyyyyfyfffyyyyyyyyyy |
| 23 | efyyyyyyyyyyyfyfyyyyyee | yyyyyyfyfffyyyyyyyyyy |
| 24 | efyyyyyyyyyyyfyfyyyyyyy | eeyyyyfyfffyyyyyyyyee |
| 25 | efyyyyyyyyyyyfyfyyyyyee | eeyyyyyyyffyyyyyyyyee |
| 26 | efyyyyyyyyyyyfyfyyyyyyy | yyyyyyyyyffyyyyyyyyyy |
| 27 | efyyyyyyyyyyyfyfyyyyyee | yyyyyyyyyffyyyyyyyyyy |
| 28 | efyyyyyyyyyyyfyfyyyyyyy | eeyyyyyyyffyyyyyyyyee |
| 29 | efyyyyyyyyyyyfyfyyyyyee | eeyyyyfyfyfyyyyyyyyee |
| 30 | efyyyyyyyyyyyfyfyyyyyyy | yyyyyyfyfyfyyyyyyyyyy |
| 31 | efyyyyyyyyyyyfyfyyyyyee | yyyyyyfyfyfyyyyyyyyyy |
| 32 | efyyyyyyyyyyyfyfyyyyyyy | eeyyyyfyfyfyyyyyyyyee |
| 33 | efyyyfyyyyyyyfyfyyyyyyy | yyyyyyyyyffyyyyyyyyyy |
| 34 | efyyyfyyyyyyyfyfyyyyyee | eeyyyyyyyffyyyyyyyyee |
| 35 | efyyyfyyyyyyyfyfyyyyyyy | eeyyyyyyyffyyyyyyyyee |
| 36 | efyyyfyyyyyyyfyfyyyyyee | yyyyyyyyyffyyyyyyyyyy |
| 37 | efyyyfyyyyyyyfyfyyyyyyy | yyyyyyfyfyfyyyyyyyyyy |
| 38 | efyyyfyyyyyyyfyfyyyyyee | eeyyyyfyfyfyyyyyyyyee |
| 39 | efyyyfyyyyyyyfyfyyyyyyy | eeyyyyfyfyfyyyyyyyyee |
| 40 | efyyyfyyyyyyyfyfyyyyyee | yyyyyyfyfyfyyyyyyyyyy |
| 41 | efyyyfyyyyyyyfyfyyyee | eeyyyyfyfffyyyyyyee |
| 42 | efyyyfyyyyyyyfyfyyyyy | yyyyyyfyfffyyyyyyyy |
| 43 | efyyyfyyyyyyyfyfyyyee | yyyyyyfyfffyyyyyyyy |
| 44 | efyyyfyyyyyyyfyfyyyyy | eeyyyyfyfffyyyyyyee |
| 45 | efyyyfyyyyyyyfyfyyyee | eeyyyyffyyyyyyyyee |
| 46 | efyyyfyyyyyyyfyfyyyyy | yyyyyyffyyyyyyyyyy |
| 47 | efyyyfyyyyyyyfyfyyyee | yyyyyyffyyyyyyyyyy |
| 48 | efyyyfyyyyyyyfyfyyyyy | eeyyyyffyyyyyyyyee |
| 49 | e[FHNA]yydydyyyyfyfyfyyyyyee | eeyyyyfyfffyyyyyyyyee |
| 50 | efyyyfyyyyyyyfyfyyyyyey | eeyyyyfyfffyyyyyyyyee |
| 51 | efyyyfyyyyyyyfyfyyyyyey | yyyyyyfyfffyyyyyyyyyy |
| 52 | e[FHNA]yydydyyyyfyfyfyyyyyyy | yyyyyyfyfffyyyyyyyyyy |
| 53 | e[FHNA]yydydyyyyfyfyfyyyyyee | yyyyyyfyfffyyyyyyyyyy |
| 54 | e[FHNA]yydydyyyyfyfyfyyyyyyy | eeyyyyfyfffyyyyyyyyee |
| 55 | e[FHNA]yydydyyyyfyfyfyyyyyee | eeyyyyfyfyfyyyyyyyyee |
| 56 | e[FHNA]yydydyyyyfyfyfyyyyyyy | yyyyyyfyfyfyyyyyyyyyy |
| 57 | e[FHNA]yydydyyyyfyfyfyyyyyee | yyyyyyfyfyfyyyyyyyyyy |
| 58 | e[FHNA]yydydyyyyfyfyfyyyyyyy | eeyyyyfyfyfyyyyyyyyee |
| 59 | efyyyfyyyyyyyfyfyyyyyyy | yyyyyyfyfffyyyyyyyyyy |
| 60 | efyyyfyyyyyyyfyfyyyyyee | eeyyyyfyfffyyyyyyyyee |
| 61 | efyyyfyyyyyyyfyfyyyyyee | yyyyyyfyfffyyyyyyyyyy |
| 62 | efyyyfyyyyyyyfyfyyyyyyy | eeyyyyfyfffyyyyyyyyee |
| 63 | efyyyfyyyyyyyfyfyyyyyyy | yyyyyyyyyyyyyyyyyyyyy |
| 64 | efyyyfyyyyyyyfyfyyyyyee | eeyyyyyyyyyyyyyyyyyee |
| 65 | efyyyfyyyyyyyfyfyyyyyyy | eeyyyyyyyyyyyyyyyyyee |
| 66 | efyyyfyyyyyyyfyfyyyyyee | yyyyyyyyyyyyyyyyyyyyy |
| 67 | efyyyfyyyyyyyfyfyyyyyyy | yyyyyyyyyydyyyyyyyyyy |
| 68 | efyyyfyyyyyyyfyfyyyyyee | eeyyyyyyyydyyyyyyyyee |
| 69 | efyyyfyyyyyyyfyfyyyyyyy | eeyyyyyyyydyyyyyyyyee |
| 70 | efyyyfyyyyyyyfyfyyyyyee | yyyyyyyyyydyyyyyyyyyy |
| 71 | efyyyyyyyyyyyfyyyyyyyee | eeyyyyfyfffyyyyyyyyee |
| 72 | efyyyyyyyyyyyfyyyyyyyyy | yyyyyyfyfffyyyyyyyyyy |
| 73 | efyyyyyyyyyyyfyyyyyyyee | yyyyyyfyfffyyyyyyyyyy |
| 74 | efyyyyyyyyyyyfyyyyyyyyy | eeyyyyfyfffyyyyyyyyee |
| 75 | efyyyyyyyyyyyfyyyyyyyee | eeyyyyfyfyfyyyyyyyyee |
| 76 | efyyyyyyyyyyyfyyyyyyyyy | yyyyyyfyfyfyyyyyyyyyy |
| 77 | efyyyyyyyyyyyfyyyyyyyee | yyyyyyfyfyfyyyyyyyyyy |
| 78 | efyyyyyyyyyyyfyyyyyyyyy | eeyyyyfyfyfyyyyyyyyee |
| 79 | efyyydyyyyyyyfyfyyyyyee | eeyyyyfyfffyyyyyyyyee |
| 80 | efyyydyyyyyyyfyfyyyyyyy | yyyyyyfyfffyyyyyyyyyy |
| 81 | efyyydyyyyyyyfyfyyyyyee | yyyyyyfyfffyyyyyyyyyy |
| 82 | efyyydyyyyyyyfyfyyyyyyy | eeyyyyfyfffyyyyyyyyee |
| 83 | efyyyfyyyyyyyfyfyyyee | eeyyfyfffyyyyyyyyee |
| 84 | efyyyfyyyyyyyfyfyyyyy | yyyyfyfffyyyyyyyyyy |
| 85 | efyyyfyyyyyyyfyfyyyee | yyyyfyfffyyyyyyyyyy |
| 86 | efyyyfyyyyyyyfyfyyyyy | eeyyfyfffyyyyyyyyee |
| 87 | efyyyfyyyyyyyfydyyyyyee | eeyyyyfyfffyyyyyyyyee |
| 88 | efyyyfyyyyyyyfydyyyyyyy | yyyyyyfyfffyyyyyyyyyy |
| 89 | efyyyfyyyyyyyfydyyyyyee | yyyyyyfyfffyyyyyyyyyy |
| 90 | efyyyfyyyyyyyfydyyyyyyy | eeyyyyfyfffyyyyyyyyee |
| 91 | e[FHNA]yyyfyyyyyyyfyfyyyyyee | eeyyyyfyfffyyyyyyyyee |
| 92 | e[FHNA]yyyfyyyyyyyfyfyyyyyyy | yyyyyyfyfffyyyyyyyyyy |
| 93 | e[FHNA]yyyfyyyyyyyfyfyyyyyee | yyyyyyfyfffyyyyyyyyyy |
| 94 | e[FHNA]yyyfyyyyyyyfyfyyyyyyy | eeyyyyfyfffyyyyyyyyee |
| 95 | efyyyyyyyyyyyfyyyyyyyee | eeyyyyyyyffyyyyyyyyee |
| 96 | efyyyyyyyyyyyfyyyyyyyyy | yyyyyyyyyffyyyyyyyyyy |
| 97 | efyyyyyyyyyyyfyyyyyyyee | yyyyyyyyyffyyyyyyyyyy |
| 98 | efyyyyyyyyyyyfyyyyyyyyy | eeyyyyyyyffyyyyyyyyee |
| 99 | efyyyfyyyyyyyfyyyyyyyee | eeyyyyfyfffyyyyyyyyee |
| 100 | efyyyfyyyyyyyfyyyyyyyyy | yyyyyyfyfffyyyyyyyyyy |
| 101 | efyyyfyyyyyyyfyyyyyyyee | yyyyyyfyfffyyyyyyyyyy |
| 102 | efyyyfyyyyyyyfyyyyyyyyy | eeyyyyfyfffyyyyyyyyee |
| 103 | efyyyfyyyyyyyfyyyyyyyee | eeyyyyyyyffyyyyyyyyee |
| 104 | efyyyfyyyyyyyfyyyyyyyyy | yyyyyyyyyffyyyyyyyyyy |
| 105 | efyyyfyyyyyyyfyyyyyyyee | yyyyyyfyfffyyyyyyyyyy |
| 106 | efyyyfyyyyyyyfyyyyyyyyy | eeyyyyfyfffyyyyyyyyee |
| 107 | efyyyyyyyyyyyfyfyyyyye | eeyyyyyyyffyyyyyyyyee |
| 108 | efyyyyyyyyyyyfyfyyyyyy | yyyyyyyyyffyyyyyyyyyy |
| 109 | efyyyyyyyyyyyfyfyyyyye | yyyyyyyyyffyyyyyyyyyy |
| 110 | efyyyyyyyyyyyfyfyyyyyy | eeyyyyyyyffyyyyyyyyee |
| 111 | efyyyyyyyyyyyfyfyyyyye | eeyyyyfyfyfyyyyyyyyee |
| 112 | efyyyyyyyyyyyfyfyyyyyy | yyyyyyfyfyfyyyyyyyyyy |
| 113 | efyyyyyyyyyyyfyfyyyyye | yyyyyyfyfyfyyyyyyyyyy |
| 114 | efyyyyyyyyyyyfyfyyyyyy | eeyyyyfyfyfyyyyyyyyee |
| 115 | efyyyyyyyyyyyfyfyyyyy | eeyyyyyyyffyyyyyyyyee |
| 116 | efyyyyyyyyyyyfyfyyyyy | yyyyyyyyyffyyyyyyyyyy |
| 117 | efyyyyyyyyyyyfyfyyyyy | eeyyyyfyfyfyyyyyyyyee |
| 118 | efyyyyyyyyyyyfyfyyyyy | yyyyyyfyfyfyyyyyyyyyy |
| 119 | efyyyfyyyyyyyfyyyyyyye | eeyyyyfyfffyyyyyyyyee |
| 120 | efyyyfyyyyyyyfyyyyyyyy | yyyyyyfyfffyyyyyyyyyy |
| 121 | efyyyfyyyyyyyfyyyyyyye | yyyyyyfyfffyyyyyyyyyy |
| 122 | efyyyfyyyyyyyfyyyyyyyy | eeyyyyfyfffyyyyyyyyee |
| 123 | efyydydyyyyyyfyfyyyyye | eeyyyyfyfyfyyyyyyyyee |
| 124 | efyydydyyyyyyfyfyyyyyy | yyyyyyfyfyfyyyyyyyyyy |
| 125 | efyydydyyyyyyfyfyyyyye | yyyyyyfyfyfyyyyyyyyyy |
| 126 | efyydydyyyyyyfyfyyyyyy | eeyyyyfyfyfyyyyyyyyee |
| 127 | efyydydyyyyyyfyfyyyyy | eeyyyyfyfyfyyyyyyyyee |
| 128 | efyydydyyyyyyfyfyyyyy | yyyyyyfyfyfyyyyyyyyyy |
| 129 | efyydydyyyyyyfyfyyyyye | eeyyyyyyyffyyyyyyyyee |
| 130 | efyydydyyyyyyfyfyyyyyy | yyyyyyyyyffyyyyyyyyyy |
| 131 | efyydydyyyyyyfyfyyyyye | yyyyyyyyyffyyyyyyyyyy |
| 132 | efyydydyyyyyyfyfyyyyyy | eeyyyyyyyffyyyyyyyyee |
| 133 | efyydydyyyyyyfyfyyyyy | eeyyyyyyyffyyyyyyyyee |
| 134 | efyydydyyyyyyfyfyyyyy | yyyyyyyyyffyyyyyyyyyy |
| 135 | efyydydyyyyyyfyfyyyyye | eeyyyyfyfffyyyyyyyyee |
| 136 | efyydydyyyyyyfyfyyyyyy | yyyyyyfyfffyyyyyyyyyy |
| 137 | efyydydyyyyyyfyfyyyyye | yyyyyyfyfffyyyyyyyyyy |
| 138 | efyydydyyyyyyfyfyyyyyy | eeyyyyfyfffyyyyyyyyee |
| 139 | efyydydyyyyyyfyfyyyyy | eeyyyyfyfffyyyyyyyyee |
| 140 | efyydydyyyyyyfyfyyyyy | yyyyyyfyfffyyyyyyyyyy |
| 141 | efyyydyyyyyyyfyfyyyyye | eeyyyyyyyffyyyyyyyyee |
| 142 | efyyydyyyyyyyfyfyyyyyy | yyyyyyyyyffyyyyyyyyyy |
| 143 | efyyydyyyyyyyfyfyyyyye | yyyyyyyyyffyyyyyyyyyy |
| 144 | efyyydyyyyyyyfyfyyyyyy | eeyyyyyyyffyyyyyyyyee |
| 145 | efyyydyyyyyyyfyfyyyyy | eeyyyyyyyffyyyyyyyyee |
| 146 | efyyydyyyyyyyfyfyyyyy | yyyyyyyyyffyyyyyyyyyy |
| 147 | efyyydyyyyyyyfyfyyyyye | eeyyyyfyfyfyyyyyyyyee |
| 148 | efyyydyyyyyyyfyfyyyyyy | yyyyyyfyfyfyyyyyyyyyy |
| 149 | efyyydyyyyyyyfyfyyyyye | yyyyyyfyfyfyyyyyyyyyy |
| 150 | efyyydyyyyyyyfyfyyyyyy | eeyyyyfyfyfyyyyyyyyee |
| 151 | efyyydyyyyyyyfyfyyyyy | eeyyyyfyfyfyyyyyyyyee |
| 152 | efyyydyyyyyyyfyfyyyyy | yyyyyyfyfyfyyyyyyyyyy |
| 153 | efyyydyyyyyyyfyfyyyyye | eeyyyyfyfffyyyyyyyyee |
| 154 | efyyydyyyyyyyfyfyyyyyy | yyyyyyfyfffyyyyyyyyyy |
| 155 | efyyydyyyyyyyfyfyyyyye | yyyyyyfyfffyyyyyyyyyy |
| 156 | efyyydyyyyyyyfyfyyyyyy | eeyyyyfyfffyyyyyyyyee |
| 157 | efyyydyyyyyyyfyfyyyyy | eeyyyyfyfffyyyyyyyyee |
| 158 | efyyydyyyyyyyfyfyyyyy | yyyyyyfyfffyyyyyyyyyy |

In some embodiments, an oligomeric duplex having a reduced fluorine content provided herein contains a first modified oligonucleotide (e.g., an antisense oligonucleotide) having a reduced fluorine content having a first sugar motif (1^{st}) and a second modified oligonucleotide (e.g., a sense oligonucleotide) having a reduced fluorine content having a second sugar motif (2^{nd}) and the first and second sugar motifs are independently selected from among the first and second sugar motifs provided in Table C. In some such embodiments, the number of nucleosides in the first modified oligonucleotide is 23, 22, or 21 and the number of nucleosides in the second modified oligonucleotide is 21, 20, or 19. In some such embodiments, the number of nucleosides in the first modified oligonucleotide is 23 and the number of nucleosides in the second modified oligonucleotide is 21. In some embodiments, the number of nucleosides in the first modified oligonucleotide is 21 and the number of nucleosides in the second modified oligonucleotide is 19. In some such embodiments, the first modified oligonucleotide (e.g., an antisense oligonucleotide) comprises or consists of a nucleobase sequence selected from among the sequences provided in Table A, Table B1 or SEQ ID NOS: 3-314, 627-782, 939-976, 1033-1038, 1254-1255, and 1258-1287 (or at least 21 contiguous nucleosides of any one of the sequences provided in Table A, Table B1 or SEQ ID NOS: 3-314, 627-782, 939-976, 1033-1038, 1254-1255, and 1258-1287) and/or the nucleobase sequence of the second modified oligonucleotide (e.g., a sense oligonucleotide) comprises or consists of a nucleobase sequence complementary to a sequence in the nucleobase sequence of the first modified oligonucleotide, or of a nucleobase sequence selected from among the sequences provided in Table A, Table B2 or SEQ ID NOS: 315-626, 783-938, 977-1016, 1027-1032, 1256-1257, and 1288-1290 (or at least 19 contiguous nucleosides of any one of the sequences provided in Table A, Table B2 or SEQ ID NOS: 315-626, 783-938, 977-1016, 1027-1032, 1256-1257, and 1288-1290). In some such embodiments, the first modified oligonucleotide (e.g., an antisense oligonucleotide) comprises or consists of a nucleobase sequence selected from among SEQ ID NOS: 29,171,176, 967, 968, 971, 972, 974, 975, 1033-1038, 1254-1255, and 1258-1287 (or at least 21 contiguous nucleosides of any one of SEQ ID NOS: 29,171,176, 967, 968, 971, 972, 974, 975, 1033-1038, 1254-1255, and 1258-1287) and/or the nucleobase sequence of the second modified oligonucleotide (e.g., a sense oligonucleotide) comprises or consists of a nucleobase sequence selected from among SEQ ID NOS: 341, 483, 488, 1011-1016, 1027-1032, 1256-1257, and 1288-1290 (or at least 19 contiguous nucleosides of any one of SEQ ID NOS: 341, 483, 488, 1011-1016, 1027-1032, 1256-1257, and 1288-1290). In some such embodiments, the first modified oligonucleotide comprises one or more modified internucleoside linkages. For example, the first modified oligonucleotide (e.g., an antisense oligonucleotide) may contain one or more phosphorothioate internucleoside linkages, such as, for example, 1, 2, 3, 4, 5 or 6 phosphorothioate internucleoside linkages. In some embodiments, no more than 4 of the internucleoside linkages of the first modified oligonucleotide are phosphorothioate internucleoside linkages. In some embodiments, all the internucleoside linkages that are not phosphorothioate internucleoside linkages are phosphodiester internucleoside linkages. Examples of internucleoside linkage motifs for a first modified oligonucleotide (e.g., an antisense oligonucleotide) having a first sugar motif selected from among the first sugar motifs provided in Table C include, but are not limited to, (from 5′ to 3′): ssooooooooooooooooooss, ssooosooooooooooooooss, ssoososoooooooooooooss, ssoooooooooooooooooos, ssoooooooooooooooooo, and ssooooooooooooooooss, wherein each “o” represents a phosphodiester internucleoside linkage and each “s” represents a phosphorothioate internucleoside linkage. In some such embodiments, the second modified oligonucleotide (e.g., a sense oligonucleotide) comprises one or more modified internucleoside linkages. In such embodiments, the second modified oligonucleotide may contain one or more mesyl phosphoramidate internucleoside linkages and/or one or more phosphorothioate internucleoside linkages, such as, for example, 1, 2, 3, 4, 5, or 6 phosphorothioate internucleoside linkages. In some embodiments, no more than 4 of the internucleoside linkages of the second modified oligonucleotide are phosphorothioate internucleoside linkages. In some embodiments, all the internucleoside linkages that are not phosphorothioate internucleoside linkages are phosphodiester internucleoside linkages. Examples of internucleoside linkage motifs for a second modified oligonucleotide (e.g., a sense oligonucleotide) having a second sugar motif selected from among the second sugar motifs provided in Table C include, but are not limited to, (from 5′ to 3′): ssooooooooooooooooss, ssooooooosooooooooss, ssooooooooooooooss, and ssoooooozozoooooooss, wherein each “o” represents a phosphodiester linkage, each “z” represents a mesyl phosphoramidate linkage, and each “s” represents a phosphorothioate linkage. In particular embodiments, the first modified oligonucleotide has an internucleoside linkage motif of ssooooooooooooooooooss and the second modified oligonucleotide has an internucleoside linkage motif of ssooooooooooooooooss.

In some embodiments, an oligomeric duplex having a reduced fluorine content provided herein contains a first modified oligonucleotide (e.g., an antisense oligonucleotide) having a reduced fluorine content having a first sugar motif (1^{st}) and a second modified oligonucleotide (e.g., a sense oligonucleotide) having a reduced fluorine content having a second sugar motif (2^{nd}) and the first and second sugar motifs are any one of the exemplary combinations of first and second sugar motifs set out in Table C. In some such embodiments, the number of nucleosides in the first modified oligonucleotide is 23, 22, or 21 and the number of nucleosides in the second modified oligonucleotide is 21, 20, or 19. In some such embodiments, the number of nucleosides in the first modified oligonucleotide is 23 and the number of nucleosides in the second modified oligonucleotide is 21. In some embodiments, the number of nucleosides in the first modified oligonucleotide is 21 and the number of nucleosides in the second modified oligonucleotide is 19. In some embodiments, the first modified oligonucleotide (e.g., an antisense oligonucleotide) comprises or consists of a nucleobase sequence selected from among the sequences provided in Table A, Table B1 or SEQ ID NOS: 3-314, 627-782, 939-976, 1033-1038, 1254-1255, and 1258-1287 (or at least 21 contiguous nucleosides of any one of the sequences provided in Table A, Table B1 or SEQ ID NOS: 3-314, 627-782, 939-976, 1033-1038, 1254-1255, and 1258-1287) and/or the nucleobase sequence of the second modified oligonucleotide (e.g., a sense oligonucleotide) comprises or consists of a nucleobase sequence complementary to a sequence in the nucleobase sequence of the first modified oligonucleotide, or of a nucleobase sequence selected from among the sequences provided in Table A, Table B2 or SEQ ID NOS: 315-626, 783-938, 977-1016, 1027-1032, 1256-1257, and 1288-1290 (or at least 19 contiguous nucleosides of any one of the sequences provided in Table A, Table B2 or SEQ ID NOS: 315-626, 783-938, 977-1016, 1027-1032, 1256-1257, and 1288-1290). In some such embodiments, the nucleobase sequence of the first modified oligonucleotide (e.g., an antisense oligonucleotide) comprises or consists of a nucleobase sequence selected from among SEQ ID NOS: 29,171,176, 967, 968, 971, 972, 974, 975, 1033-1038, 1254-1255, and 1258-1287 (or at least 21 contiguous nucleosides of any one of SEQ ID NOS: 29,171,176, 967, 968, 971, 972, 974, 975, 1033-1038, 1254-1255, and 1258-1287) and/or the nucleobase sequence of the second modified oligonucleotide (e.g., a sense oligonucleotide) comprises or consists of a nucleobase sequence selected from among SEQ ID NOS: 341, 483, 488, 1011-1016, 1027-1032, 1256-1257, and 1288-1290 (or at least 19 contiguous nucleosides of any one of SEQ ID NOS: 341, 483, 488, 1011-1016, 1027-1032, 1256-1257, and 1288-1290). In some such embodiments, the first modified oligonucleotide comprises one or more modified internucleoside linkages. For example, the first modified oligonucleotide may contain one or more phosphorothioate internucleoside linkages, such as, for example, 1, 2, 3, 4, 5 or 6 phosphorothioate internucleoside linkages. In some embodiments, no more than 4 of the internucleoside linkages of the first modified oligonucleotide are phosphorothioate internucleoside linkages. In some embodiments, all the internucleoside linkages that are not phosphorothioate internucleoside linkages are phosphodiester internucleoside linkages. Examples of internucleoside linkage motifs for a first modified oligonucleotide (e.g., an antisense oligonucleotide) having a first sugar motif in a combination of first and second sugar motifs selected from among the combinations provided in Table C include, but are not limited to, (from 5′ to 3′): ssooooooooooooooooooss, ssooosooooooooooooooss, ssoososoooooooooooooss, ssoooooooooooooooooos, ssoooooooooooooooooo, and ssooooooooooooooooss, wherein each “o” represents a phosphodiester internucleoside linkage and each “s” represents a phosphorothioate internucleoside linkage. In some such embodiments, the second modified oligonucleotide (e.g., a sense oligonucleotide) comprises one or more modified internucleoside linkages. In such embodiments, the second modified oligonucleotide may contain one or more mesyl phosphoramidate internucleoside linkages and/or one or more phosphorothioate internucleoside linkages, such as, for example, 1, 2, 3, 4, 5, or 6 phosphorothioate internucleoside linkages. In some embodiments, no more than 4 of the internucleoside linkages of the second modified oligonucleotide are phosphorothioate internucleoside linkages. In some embodiments, all the internucleoside linkages that are not phosphorothioate internucleoside linkages are phosphodiester internucleoside linkages. Examples of internucleoside linkage motifs for a second modified oligonucleotide (e.g., a sense oligonucleotide) having a second sugar motif in a combination of first and second sugar motifs selected from among the combinations provided in Table C include, but are not limited to, (from 5′ to 3′): ssooooooooooooooooss, ssooooooosooooooooss, ssooooooooooooooss, and ssoooooozozoooooooss, wherein each “o” represents a phosphodiester linkage, each “z” represents a mesyl phosphoramidate linkage, and each “s” represents a phosphorothioate linkage. In particular embodiments, the first modified oligonucleotide has an internucleoside linkage motif of ssooooooooooooooooooss and the second modified oligonucleotide has an internucleoside linkage motif of ssooooooooooooooooss.

a. Compound No. 1757465

Provided herein is Compound No. 1757465, which is an oligomeric duplex that consists of a first oligomeric compound containing a first modified oligonucleotide and a second oligomeric compound containing a second modified oligonucleotide attached to a conjugate group as follows.

### First Oligomeric Compound of Compound No. 1757465

The first oligomeric compound of Compound no. 1757465, which is Compound No. 1746643, has a first modified oligonucleotide having a nucleobase sequence of (from 5′ to 3′) TUUAAGAUGAGACAGAAAUUGAA (SEQ ID NO: 967), wherein each of nucleosides 1, 22 and 23 (from 5′ to 3′) comprises a 2′-MOE modified sugar moiety, each of nucleosides 2, 14 and 16 (from 5′ to 3′) comprises a 2′-fluoro modified sugar moiety, each of nucleosides 3-5, 7-13, 15 and 17-21 (from 5′ to 3′) comprises a 2′-OMe modified sugar moiety, and nucleoside 6 (from 5′ to 3′) is a 2′-deoxynucleoside, wherein each of the internucleoside linkages linking nucleosides 1 to 2, 2 to 3, 21 to 22, and 22 to 23 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, 18 to 19, 19 to 20, and 20 to 21 (from 5′ to 3′) is a phosphodiester linkage, and wherein nucleoside 1 (from 5′ to 3′) is attached to a vinyl phosphonate group.

The first oligomeric compound of Compound 1757465 is represented by the following chemical notation: vPTₑₛU_{fs}U_{yo}A_{yo}A_{yo}G_{do}A_{yo}U_{yo}G_{yo}A_{yo}G_{yo}A_{yo}C_{yo}A_{fo}G_{yo}A_{fo}A_{yo}A_{yo}U_{yo}U_{yo}G_{ys}AₑₛAₑ (SEQ ID NO: 1033); wherein
A=an adenine nucleobase,
C=a cytosine nucleobase,
G=a guanine nucleobase,
T=a thymine nucleobase,
U=a uracil nucleobase,
d=a 2′-deoxy sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate group.

### Second Oligomeric Compound of Compound No. 1757465

The second oligomeric compound of Compound No. 1757465, which is Compound No. 1757454, has a second modified oligonucleotide having a nucleobase sequence of (from 5′ to 3′) CAAUUUCUGUCUCAUCUUAAA (SEQ ID NO: 1011), wherein each of nucleosides 1, 2, 20 and 21 (from 5′ to 3′) comprises a 2′-MOE modified sugar moiety, each of nucleosides 10 and 11 (from 5′ to 3′) comprises a 2′-fluoro modified sugar moiety, and each of nucleosides 3-9, and 12-19 (from 5′ to 3′) comprises a 2′-OMe modified sugar moiety, wherein each of the internucleoside linkages linking nucleosides 1 to 2, 2 to 3, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, and 18 to 19 (from 5′ to 3′) is a phosphodiester linkage, and wherein nucleoside 1 (from 5′ to 3′) is a 5-methylcytosine attached through a 5′-phosphodiester linkage and a 6-(BCN-carbamate)hexyl conjugate linker to a conjugate moiety comprising the amino acid sequence of bicycle compound BCY17901 (SEQ ID NO: 1045) wherein each cysteine forms a covalent bond with the molecular scaffold 1,1′,1″-(1,3,5-triazinane-1,3,5-triyl)tris(2-bromoethanone) (TATB).

The second oligomeric compound of Compound 1757465 is represented by the following chemical notation: [X]₁-^{m}CₑₛAₑₛA_{yo}U_{yo}U_{yo}U_{yo}C_{yo}U_{yo}G_{yo}U_{fo}C_{fo}U_{yo}C_{yo}A_{yo}U_{yo}C_{yo}U_{yo}U_{yo}A_{ys}AₑₛAₑ (SEQ ID NO: 1039); wherein:
^{m}C=a 5-methyl cytosine nucleobase,
C=a cytosine nucleobase,
A=an adenine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
[X]₁=a conjugate group having the following structure:

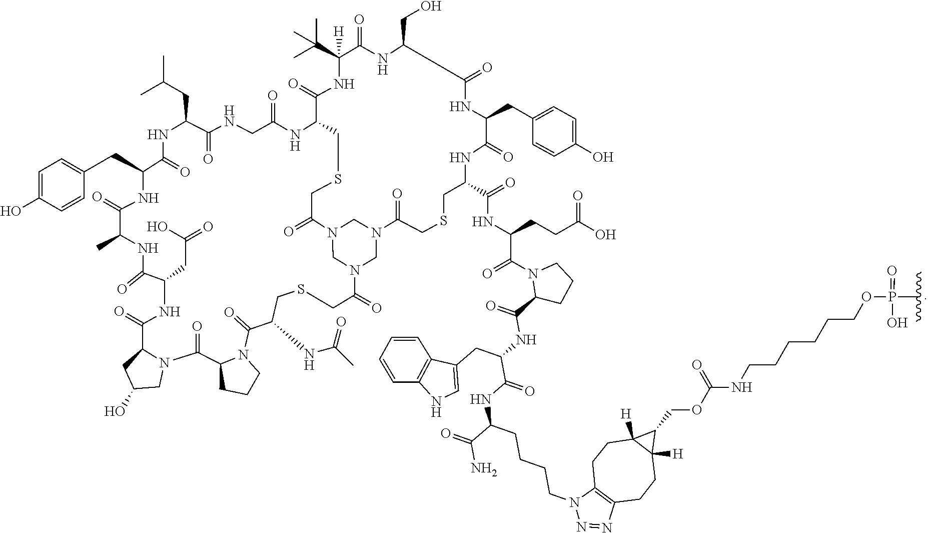 [SMILES: *P(=O)(O)OCCCCCCNC(=O)OC[C@@H]1[C@@H]2CCc3nnn(CCCC[C@H](NC(=O)[C@H](Cc4c[nH]c5ccccc45)NC(=O)[C@@H]4CCCN4C(=O)[C@H](CCC(=O)O)NC(=O)[C@@H]4CSCC(=O)N5CN6CN(C5)C(=O)CSC[C@H](NC(C)=O)C(=O)N5CCC[C@H]5C(=O)N5C[C@H](O)C[C@H]5C(=O)N[C@@H](CC(=O)O)C(=O)N[C@@H](C)C(=O)N[C@@H](Cc5ccc(O)cc5)C(=O)N[C@@H](CC(C)C)C(=O)NCC(=O)N[C@@H](CSCC6=O)C(=O)N[C@@H](C(C)(C)C)C(=O)N[C@@H](CO)C(=O)N[C@@H](Cc5ccc(O)cc5)C(=O)N4)C(N)=O)c3CC[C@@H]21]

The following chemical structure is one structural representation of Compound No. 1757465:

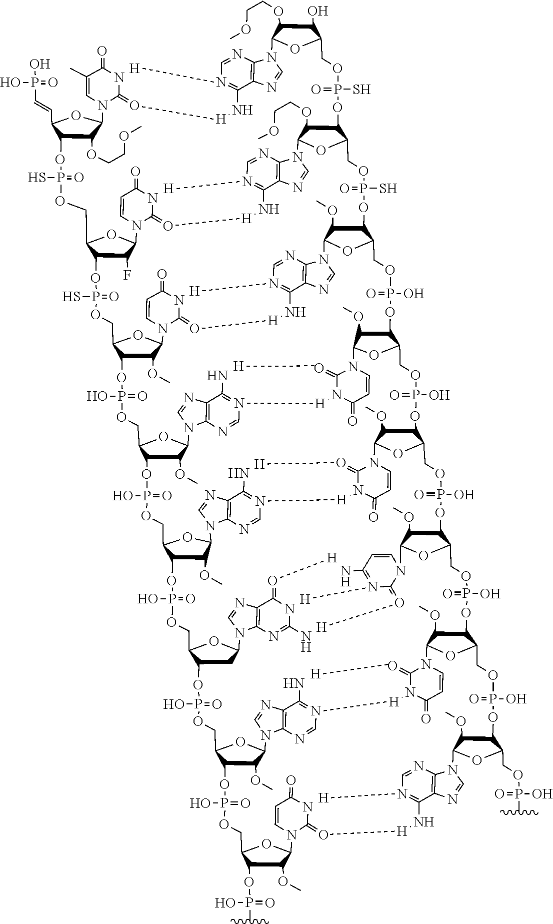

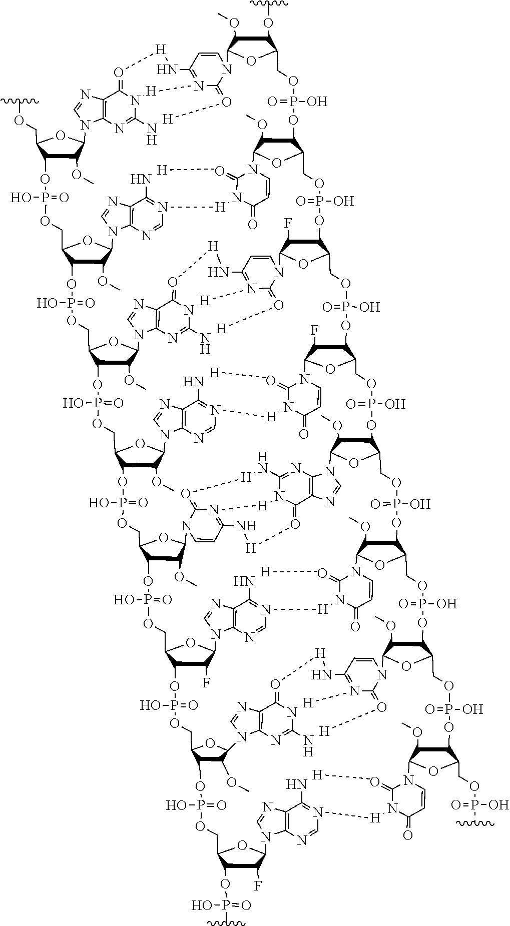

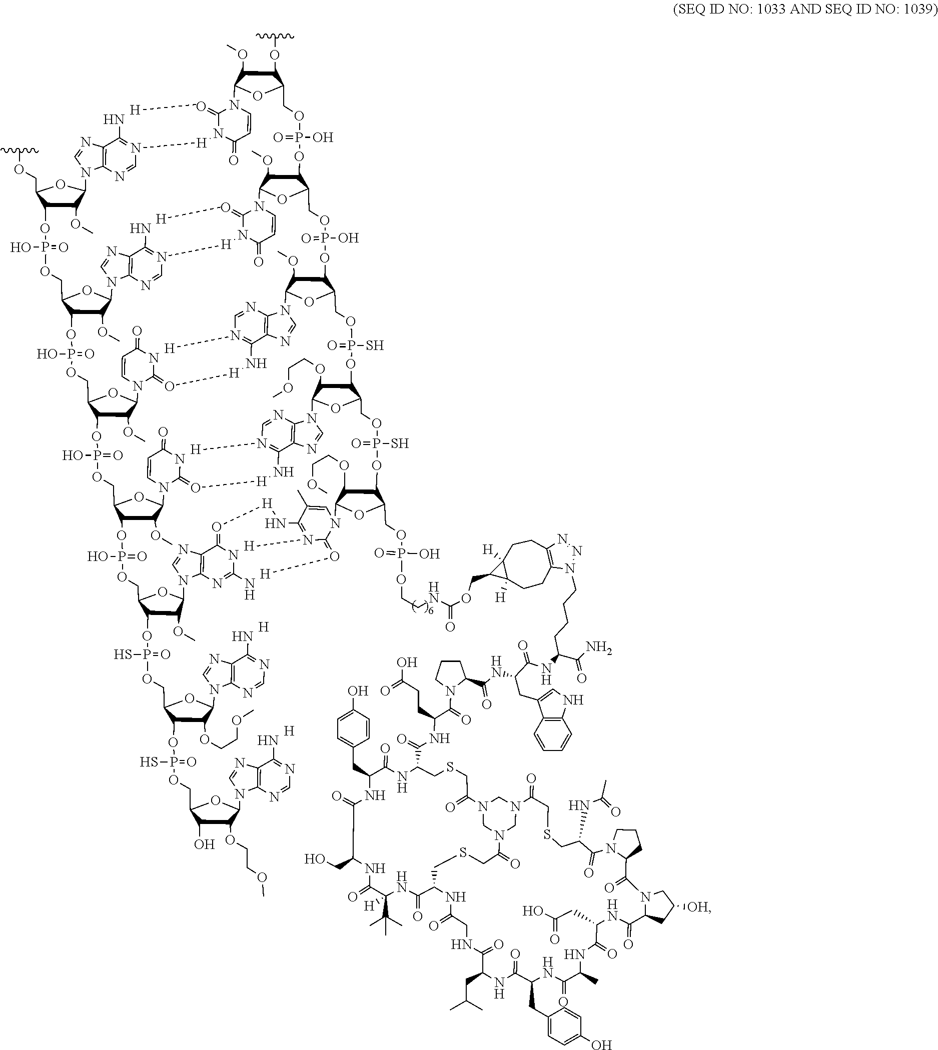

In certain embodiments, Compound No. 1757465 is in the form of an anion or a salt thereof, for example, a sodium salt. In certain embodiments, the oligomeric duplex is in anionic form in a solution. In certain embodiments, Compound No. 1757465 is a sodium salt or a potassium salt.

The following chemical structure represents Compound No. 1757465 in sodium solution:

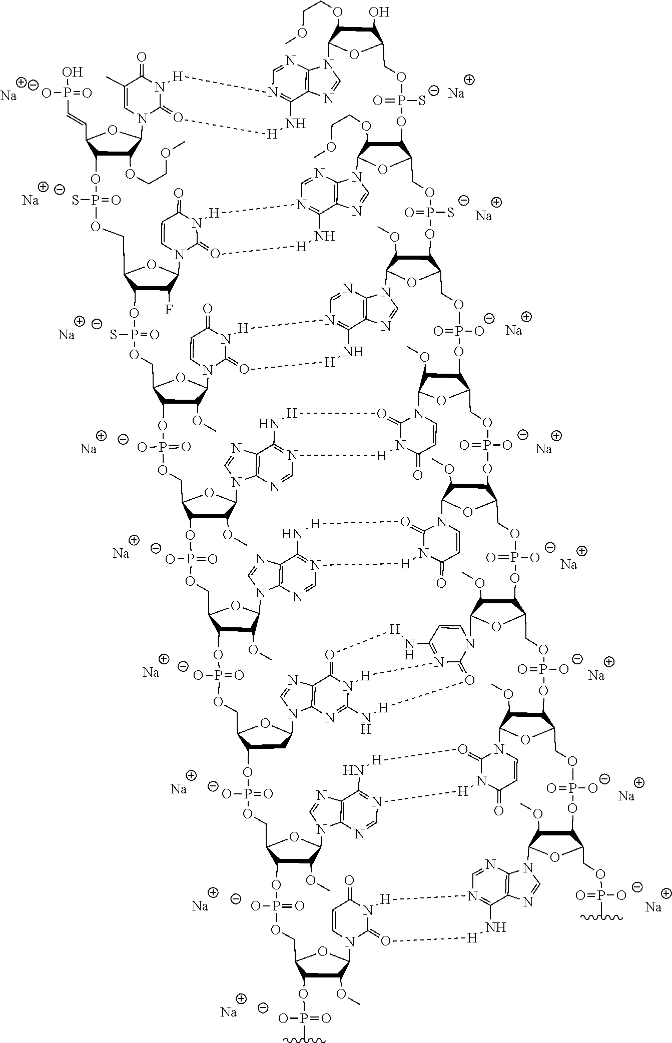

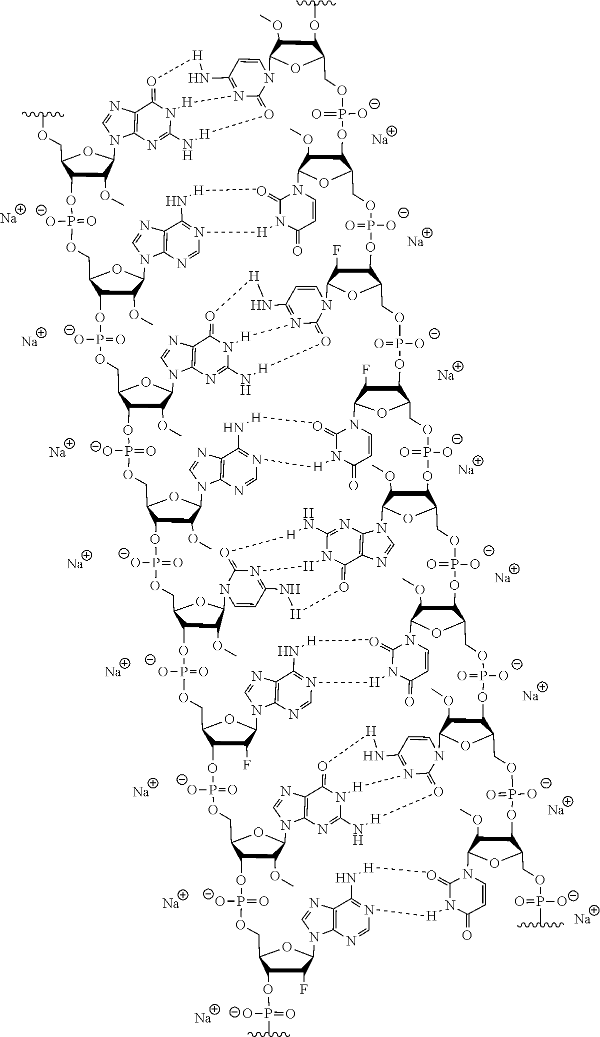

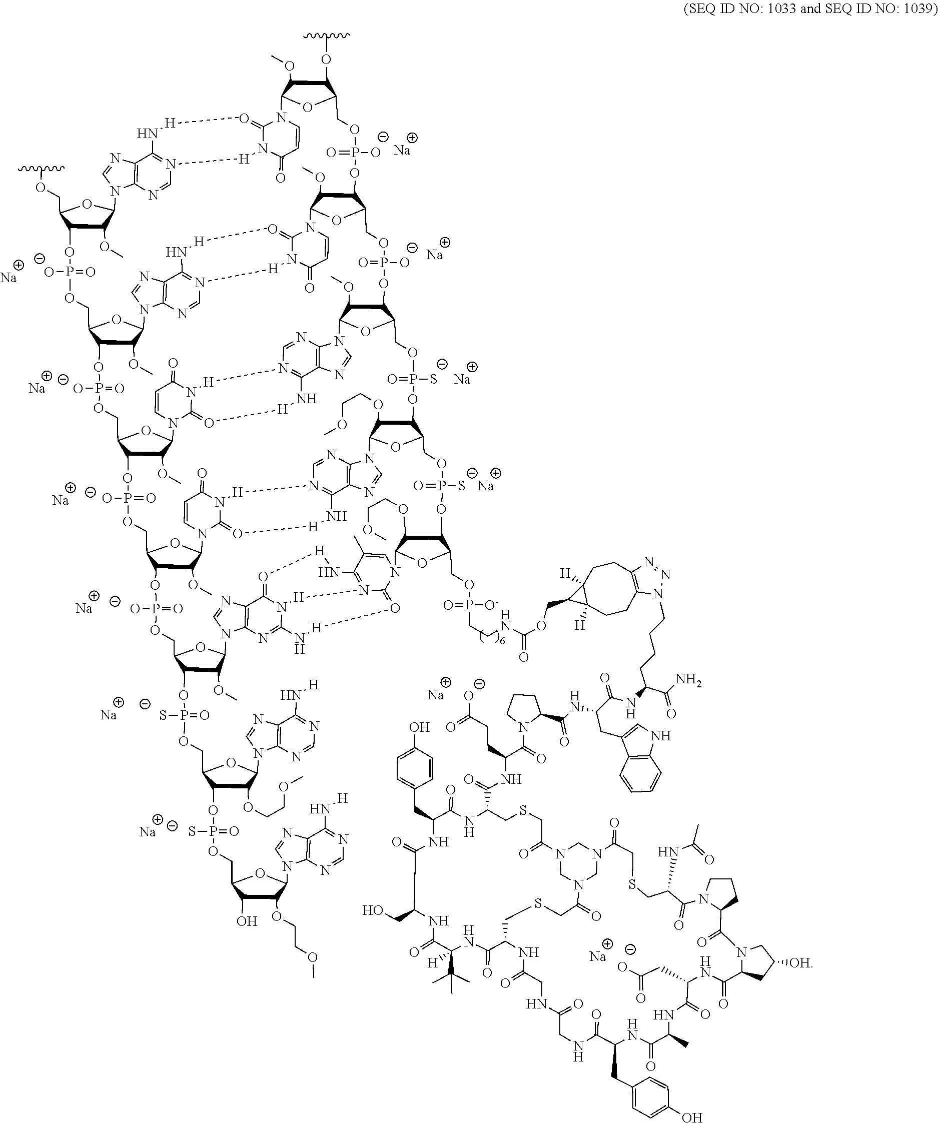

In certain embodiments, provided herein are oligomeric duplexes comprising or consisting of a first oligomeric compound containing a first modified oligonucleotide and a second oligomeric compound containing a second modified oligonucleotide as follows.

### First Oligomeric Compound

The first modified oligonucleotide has a nucleobase sequence of (from 5′ to 3′) TUUAAGAUGAGACAGAAAUUGAA (SEQ ID NO: 967), wherein each of nucleosides 1, 22 and 23 (from 5′ to 3′) comprises a 2′-MOE modified sugar moiety, each of nucleosides 2, 14 and 16 (from 5′ to 3′) comprises a 2′-fluoro modified sugar moiety, each of nucleosides 3-5, 7-13, 15 and 17-21 (from 5′ to 3′) comprises a 2′-OMe modified sugar moiety, and nucleoside 6 (from 5′ to 3′) is a 2′-deoxynucleoside, wherein each of the internucleoside linkages linking nucleosides 1 to 2, 2 to 3, 21 to 22, and 22 to 23 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, 18 to 19, 19 to 20, and 20 to 21 (from 5′ to 3′) is a phosphodiester linkage, and wherein nucleoside 1 (from 5′ to 3′) is attached to a vinyl phosphonate group.

The first oligomeric compound is represented by the following chemical notation: vPTₑₛU_{fs}U_{yo}A_{yo}A_{yo}G_{do}A_{yo}U_{yo}G_{yo}A_{yo}G_{yo}A_{yo}C_{yo}A_{fo}G_{yo}A_{fo}A_{yo}A_{yo}U_{yo}U_{yo}G_{ys}AₑₛAₑ (SEQ ID NO: 1033); wherein
A=an adenine nucleobase,
C=a cytosine nucleobase,
G=a guanine nucleobase,
T=a thymine nucleobase,
U=a uracil nucleobase,
d=a 2′-deoxy sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate group.

### Second Oligomeric Compound

The second modified oligonucleotide has a nucleobase sequence of (from 5′ to 3′) CAAUUUCUGUCUCAUCUUAAA (SEQ ID NO: 1011), wherein each of nucleosides 1, 2, 20 and 21 (from 5′ to 3′) comprises a 2′-MOE modified sugar moiety, each of nucleosides 10 and 11 (from 5′ to 3′) comprises a 2′-fluoro modified sugar moiety, and each of nucleosides 3-9, and 12-19 (from 5′ to 3′) comprises a 2′-OMe modified sugar moiety, wherein each of the internucleoside linkages linking nucleosides 1 to 2, 2 to 3, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, and 18 to 19 (from 5′ to 3′) is a phosphodiester linkage.

The second modified oligonucleotide is represented by the following chemical notation: ^{m}CₑₛAₑₛA_{yo}U_{yo}U_{yo}U_{yo}C_{yo}U_{yo}G_{yo}U_{fo}C_{fo}U_{yo}C_{yo}A_{yo}U_{yo}C_{yo}U_{yo}U_{yo}A_{ys}AₑₛAₑ (SEQ ID NO: 1027); wherein:
A=an adenine nucleobase,
^{m}C=a 5-methyl cytosine nucleobase,
C=a cytosine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

In particular embodiments, such an oligomeric duplex is represented by the following chemical structure:

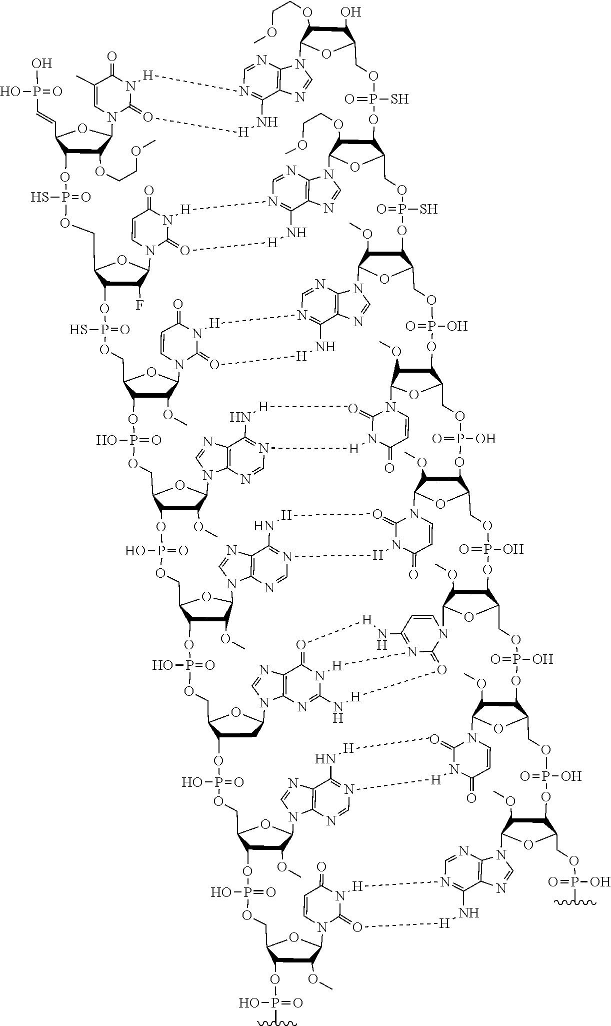

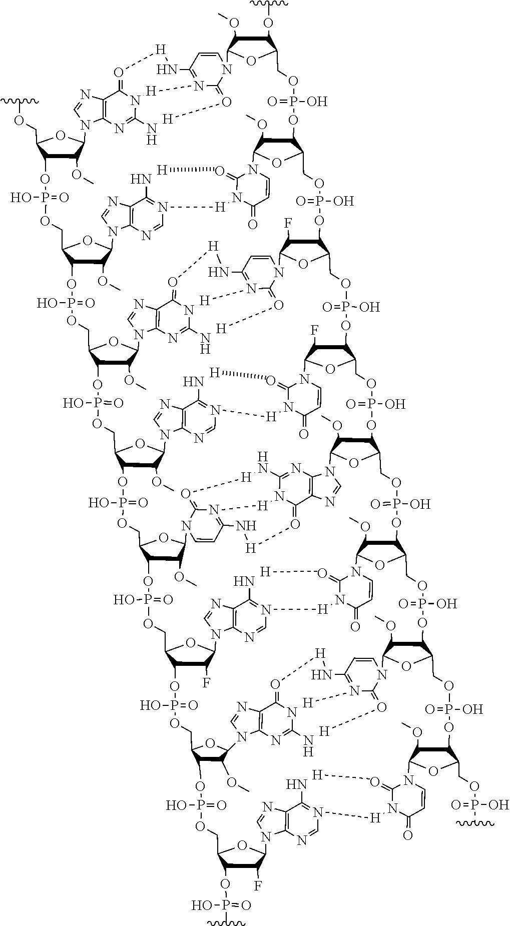

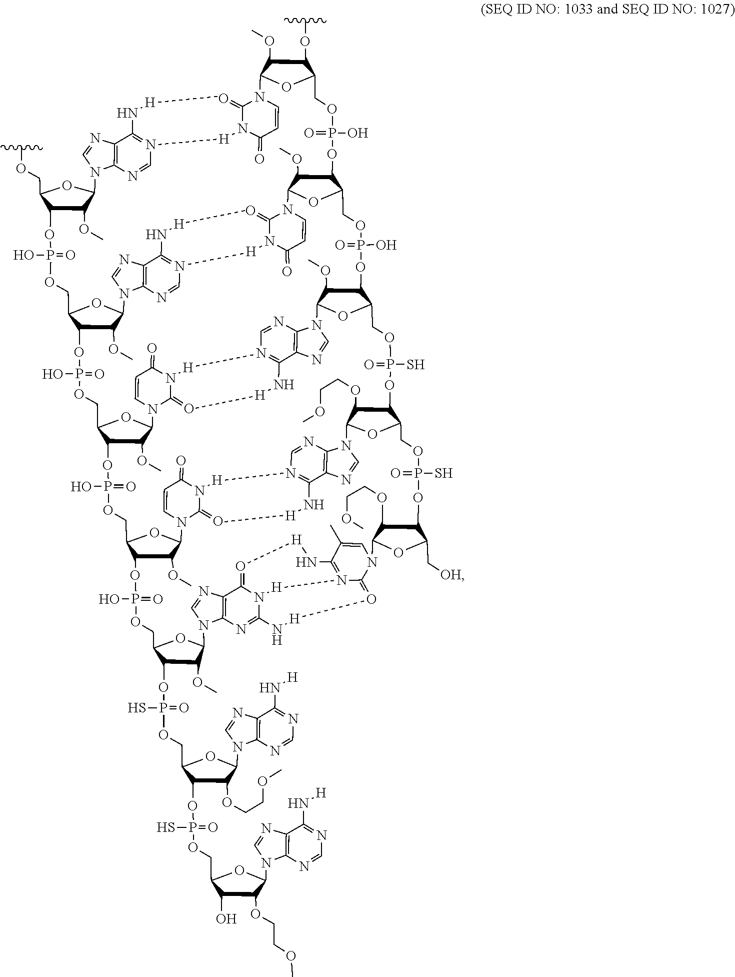

or a salt thereof.

In certain embodiments, such an oligomeric duplex is in the form of an anion or a salt thereof, for example, a sodium salt. In certain embodiments, the oligomeric duplex is in anionic form in a solution. In certain embodiments, the oligomeric duplex is a sodium salt or a potassium salt.

The following chemical structure represents such an oligomeric duplex in sodium solution:

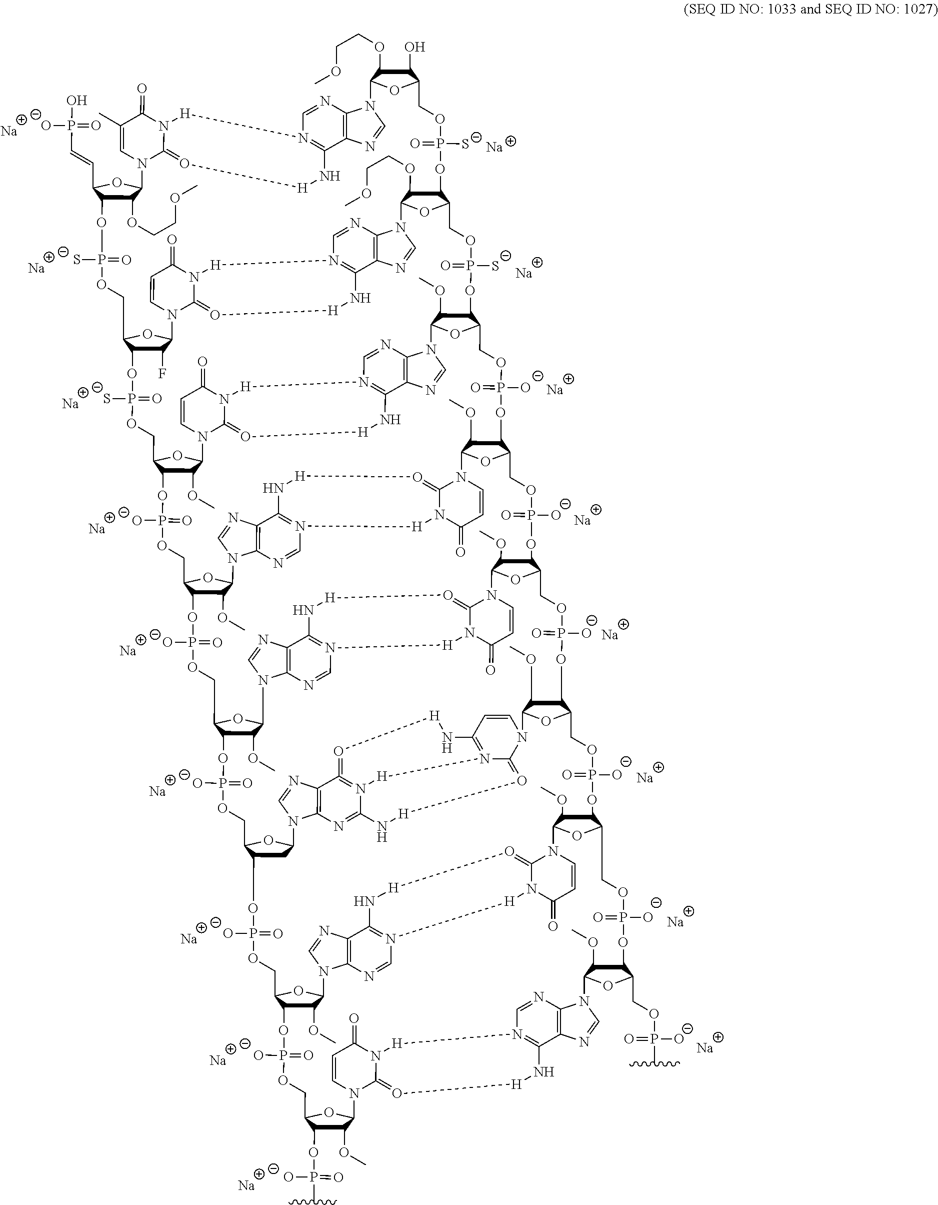

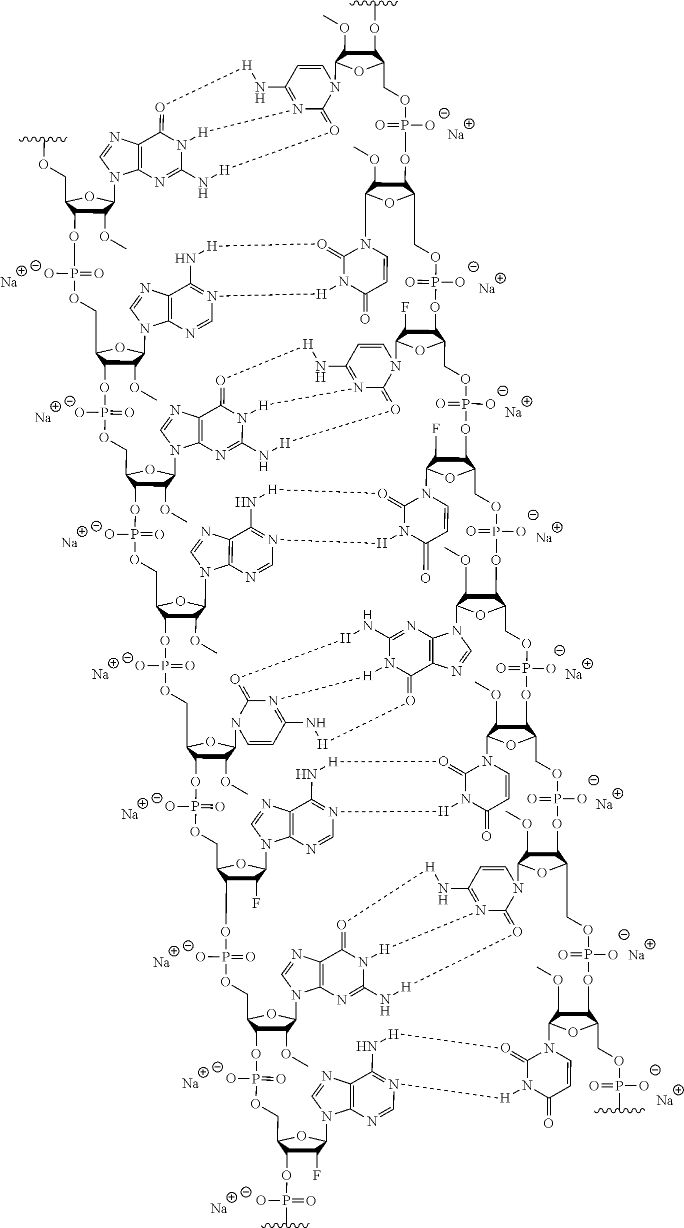

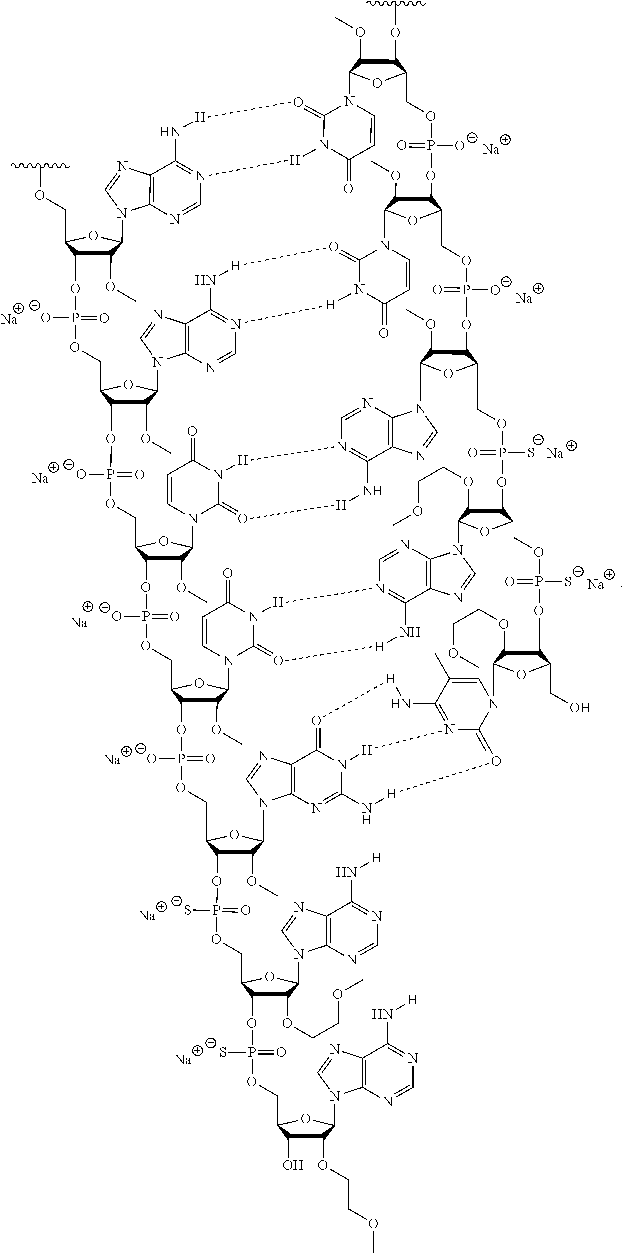

A. b. Compound No. 1757468

Provided herein is Compound No. 1757468, which is an oligomeric duplex that consists of a first oligomeric compound containing a first modified oligonucleotide and a second oligomeric compound containing a second modified oligonucleotide attached to a conjugate group as follows.

### First Oligomeric Compound of Compound No. 1757468

The first oligomeric compound of Compound No. 1757468, which is Compound No. 1746644, has a first modified oligonucleotide having a nucleobase sequence of (from 5′ to 3′) TUUAAGAUGAGACAGAAAUUGAA (SEQ ID NO: 968), wherein each of nucleosides 1, 22 and 23 (from 5′ to 3′) comprises a 2′-MOE modified sugar moiety, each of nucleosides 2, 14 and 16 (from 5′ to 3′) comprises a 2′-fluoro modified sugar moiety, each of nucleosides 3, 4, 6, 8-13, 15 and 17-21 (from 5′ to 3′) comprises a 2′-OMe modified sugar moiety, and each of nucleosides 5 and 7 (from 5′ to 3′) is a 2′-deoxynucleoside, wherein each of the internucleoside linkages linking nucleosides 1 to 2, 2 to 3, 21 to 22, and 22 to 23 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, 18 to 19, 19 to 20, and 20 to 21 (from 5′ to 3′) is a phosphodiester linkage, and wherein nucleoside 1 (from 5′ to 3′) is attached to a vinyl phosphonate group.

The first modified oligonucleotide of Compound 1757468 is represented by the following chemical notation: vPTₑₛU_{fs}U_{yo}A_{yo}A_{do}G_{yo}A_{do}U_{yo}G_{yo}A_{yo}G_{yo}A_{yo}C_{yo}A_{fo}G_{yo}A_{fo}A_{yo}A_{yo}U_{yo}U_{yo}G_{ys}AₑₛAₑ (SEQ ID NO: 1034); wherein
A=an adenine nucleobase,
C=a cytosine nucleobase,
G=a guanine nucleobase,
T=a thymine nucleobase,
U=a uracil nucleobase,
d=a 2′-deoxy sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate group.

### Second Oligomeric Compound of Compound No. 1757468

The second oligomeric compound of Compound No. 1757468, which is Compound No. 1753740, has a second modified oligonucleotide having a nucleobase sequence of (from 5′ to 3′) CAAUUUCUGUCUCAUCUUAAA (SEQ ID NO: 1012), wherein each of nucleosides 1, 2, 20 and 21 (from 5′ to 3′) comprises a 2′-MOE modified sugar moiety, each of nucleosides 7, 9, and 11 (from 5′ to 3′) comprises a 2′-fluoro modified sugar moiety, and each of nucleosides 3-6, 8, 10, and 12-19 (from 5′ to 3′) comprises a 2′-OMe modified sugar moiety, wherein each of the internucleoside linkages linking nucleosides 1 to 2, 2 to 3, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, and 18 to 19 (from 5′ to 3′) is a phosphodiester linkage, and wherein nucleoside 1 (from 5′ to 3′) is attached through a 5′-phosphodiester linkage and a 6-(BCN-carbamate)hexyl conjugate linker to a conjugate moiety comprising the amino acid sequence of bicycle compound BCY17901 (SEQ ID NO: 1045) wherein each cysteine forms a covalent bond with the molecular scaffold 1,1′,1″-(1,3,5-triazinane-1,3,5-triyl)tris(2-bromoethanone) (TATB).

The second oligomeric compound of Compound 1757468 is represented by the following chemical notation: [X]₁-CₑₛAₑₛA_{yo}U_{yo}U_{yo}U_{yo}C_{fo}U_{yo}G_{fo}U_{yo}C_{fo}U_{yo}C_{yo}A_{yo}U_{yo}C_{yo}U_{yo}U_{yo}A_{ys}AₑₛAₑ (SEQ ID NO: 1040); wherein:
C=a cytosine nucleobase,
A=an adenine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
[X]₁=a conjugate group having the following structure:

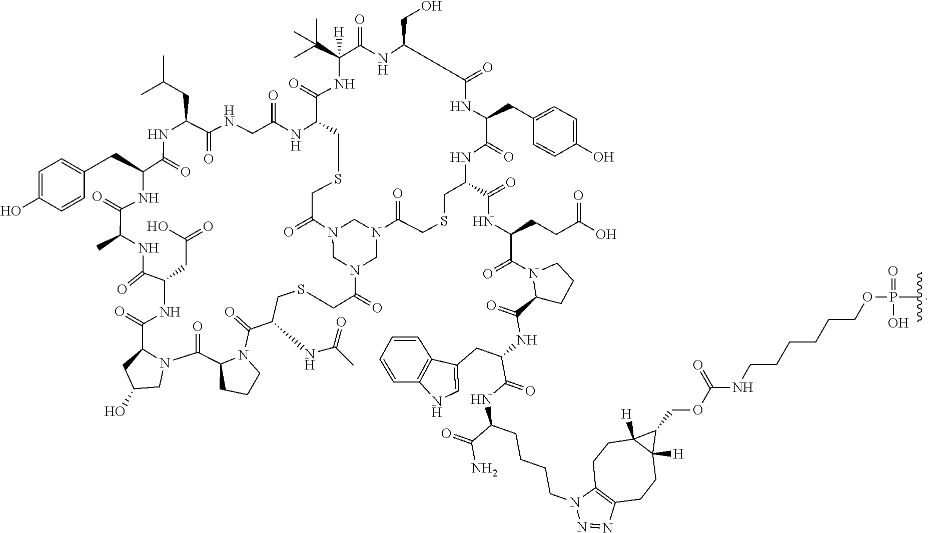 [SMILES: *P(=O)(O)OCCCCCCNC(=O)OC[C@@H]1[C@@H]2CCc3nnn(CCCC[C@H](NC(=O)[C@H](Cc4c[nH]c5ccccc45)NC(=O)[C@@H]4CCCN4C(=O)[C@H](CCC(=O)O)NC(=O)[C@@H]4CSCC(=O)N5CN6CN(C5)C(=O)CSC[C@H](NC(C)=O)C(=O)N5CCC[C@H]5C(=O)N5C[C@H](O)C[C@H]5C(=O)N[C@@H](CC(=O)O)C(=O)N[C@@H](C)C(=O)N[C@@H](Cc5ccc(O)cc5)C(=O)N[C@@H](CC(C)C)C(=O)NCC(=O)N[C@@H](CSCC6=O)C(=O)N[C@@H](C(C)(C)C)C(=O)N[C@@H](CO)C(=O)N[C@@H](Cc5ccc(O)cc5)C(=O)N4)C(N)=O)c3CC[C@@H]21]

The following chemical structure is one structural representation of Compound No. 1757468:

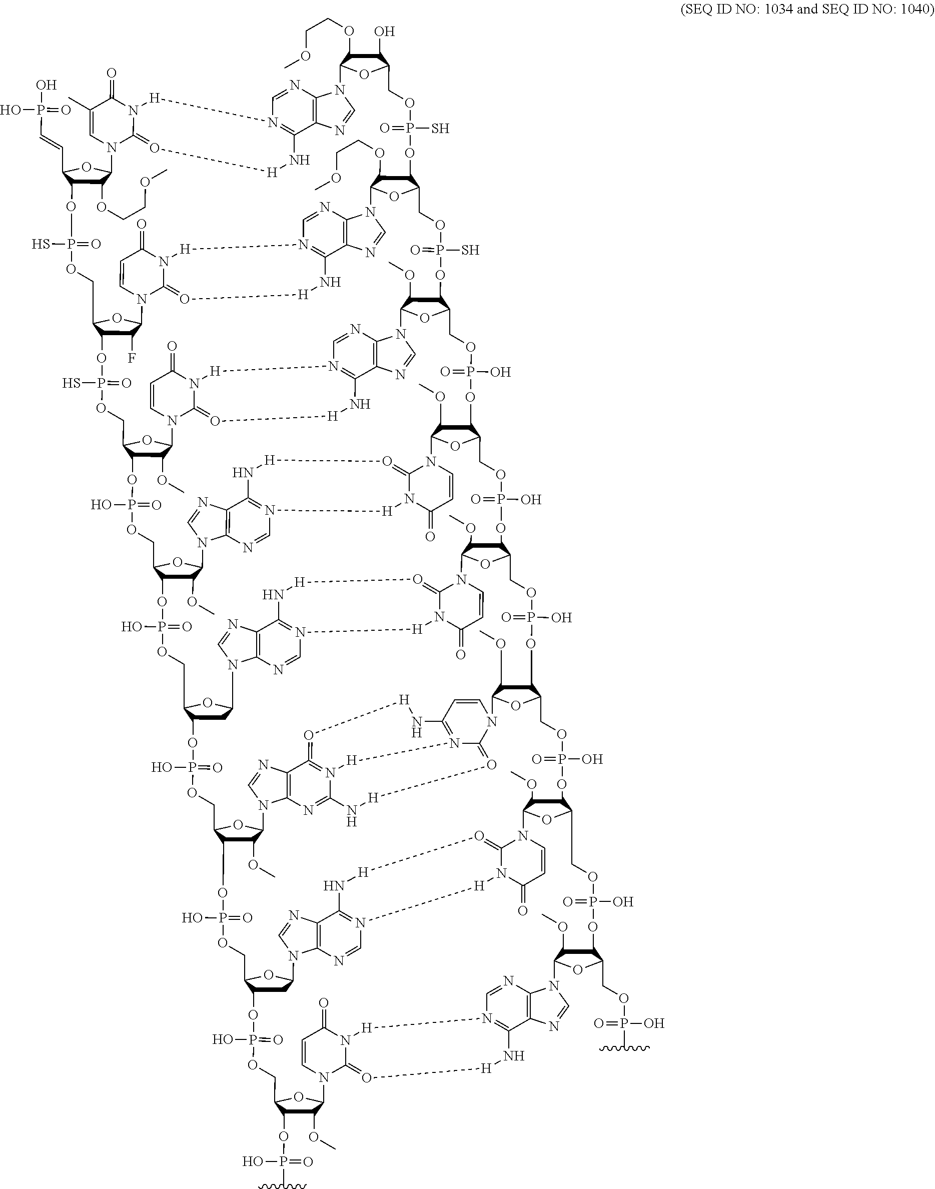

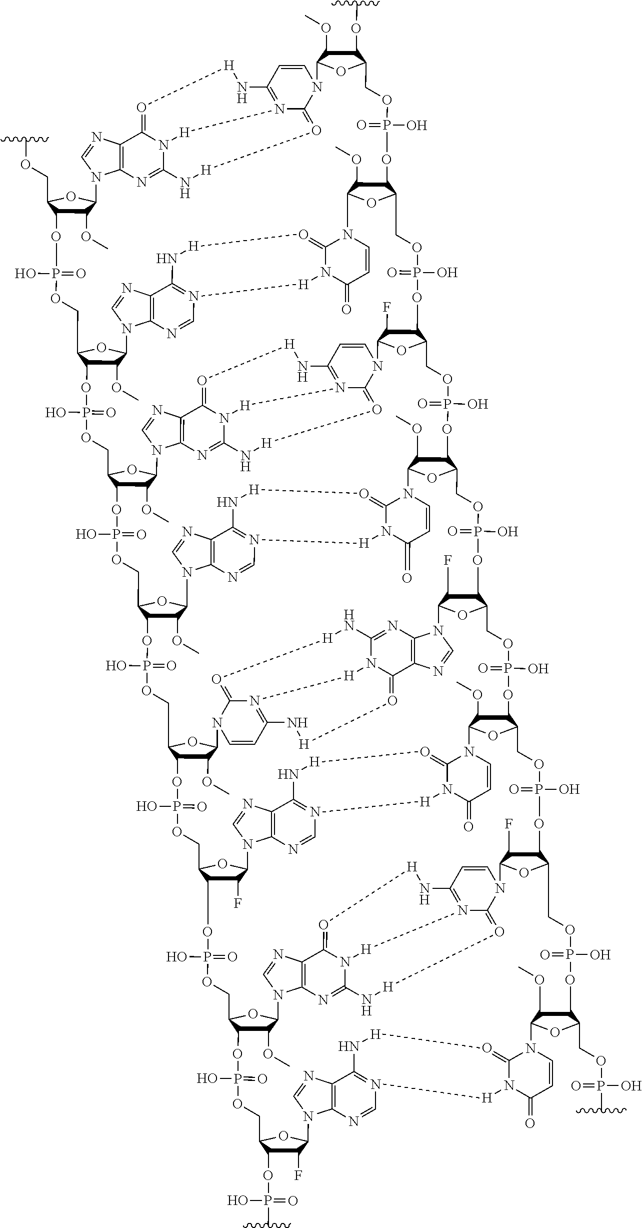

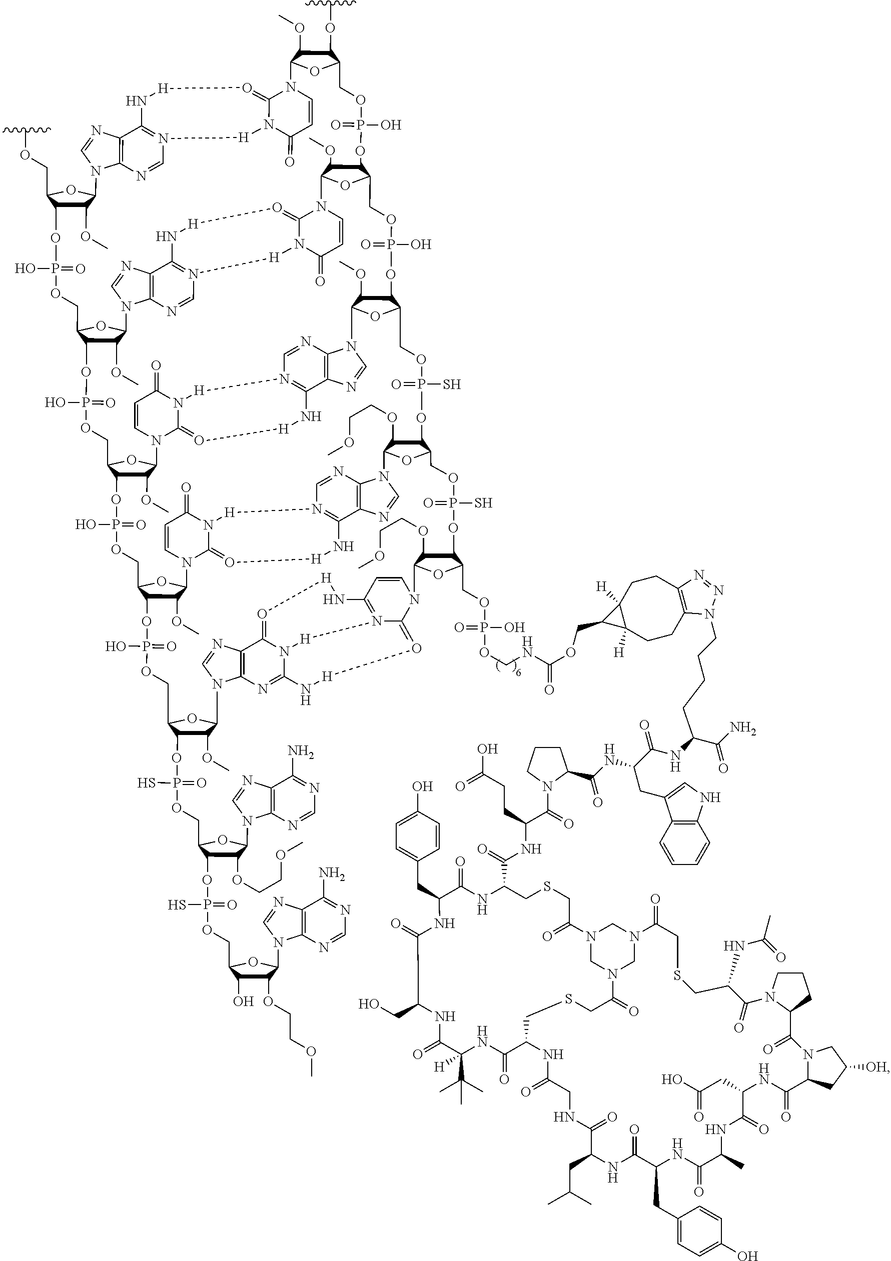

or a salt thereof.

In certain embodiments. Compound No. 1757468 is in the form of an anion or a salt thereof. for example, a sodium salt. In certain embodiments, the oligomeric duplex is in anionic form in a solution. In certain embodiments. Compound No. 1757468 is a sodium salt or a potassium salt.

The following chemical structure represents Compound No. 1757468 in sodium solution:

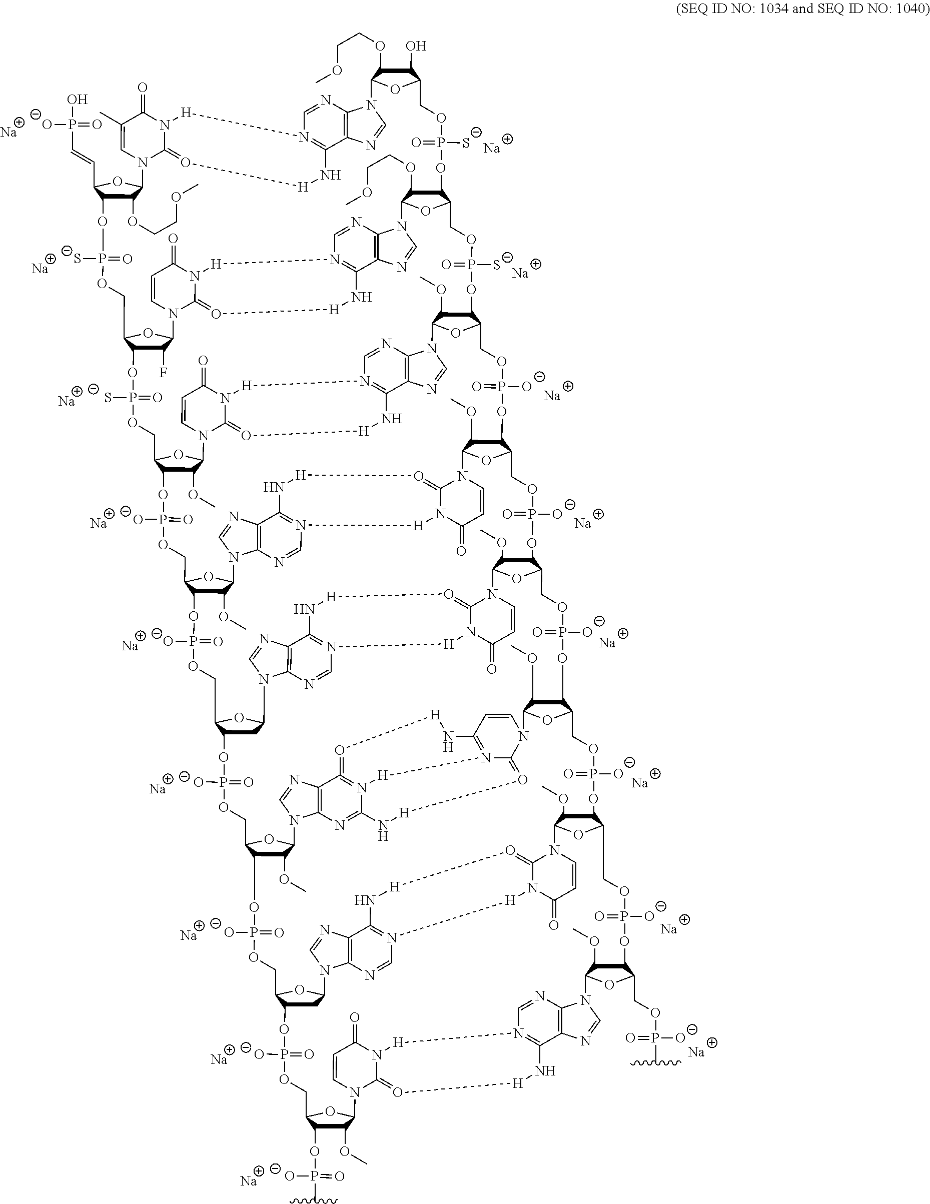

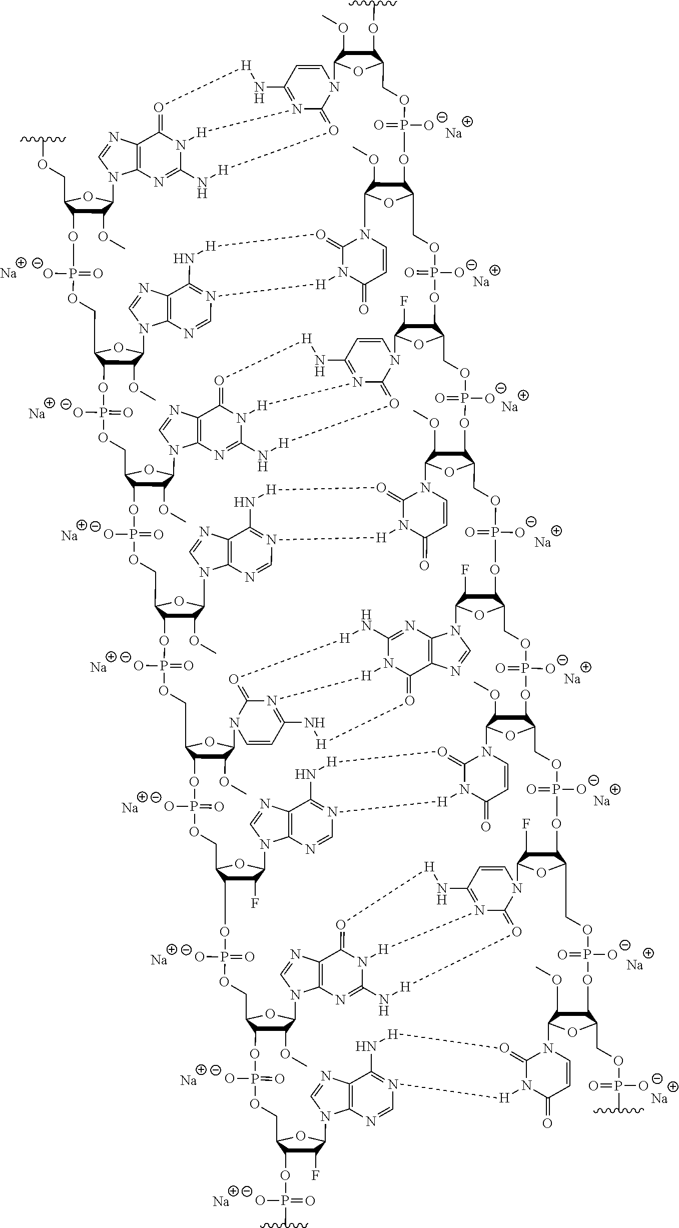

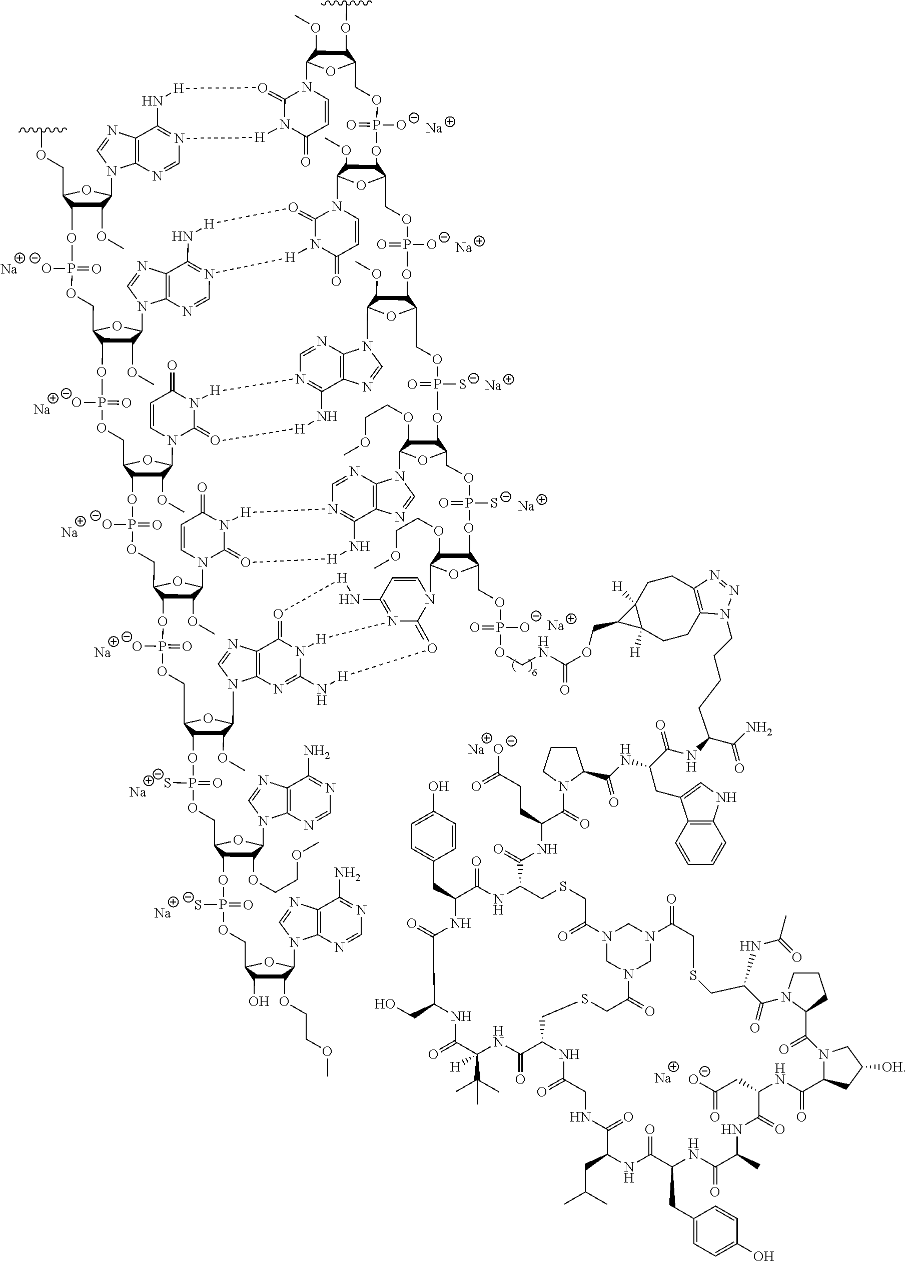

In certain embodiments. provided herein are oligomeric duplexes comprising or consisting of a first oligomeric compound containing a first modified oligonucleotide and a second oligomeric compound containing a second modified oligonucleotide as follows.

### First Oligomeric Compound

The first modified oligonucleotide has a nucleobase sequence of (from 5′ to 3′) TUUAAGAUGAGACAGAAAUUGAA (SEQ ID NO: 968), wherein each of nucleosides 1, 22 and 23 (from 5′ to 3′) comprises a 2′-MOE modified sugar moiety, each of nucleosides 2, 14 and 16 (from 5′ to 3′) comprises a 2′-fluoro modified sugar moiety, each of nucleosides 3, 4, 6, 8-13, 15 and 17-21 (from 5′ to 3′) comprises a 2′-OMe modified sugar moiety, and each of nucleosides 5 and 7 (from 5′ to 3′) is a 2′-deoxynucleoside, wherein each of the internucleoside linkages linking nucleosides 1 to 2, 2 to 3, 21 to 22, and 22 to 23 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, 18 to 19, 19 to 20, and 20 to 21 (from 5′ to 3′) is a phosphodiester linkage, and wherein nucleoside 1 (from 5′ to 3′) is attached to a vinyl phosphonate group.

The first oligomeric compound is represented by the following chemical notation:

(SEQ ID NO: 1034)
vPTₑₛU_{fs}U_{yo}A_{yo}A_{do}G_{yo}A_{do}U_{yo}G_{yo}A_{yo}G_{yo}A_{yo}C_{yo}A_{fo}G_{yo}A_{fo}
A_{yo}A_{yo}U_{yo}U_{yo}G_{ys}AₑₛAₑ;

wherein
A=an adenine nucleobase,
C=a cytosine nucleobase,
G=a guanine nucleobase,
T=a thymine nucleobase,
U=a uracil nucleobase,
d=a 2′-deoxy sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate group.

### Second Oligomeric Compound

The second modified oligonucleotide has a nucleobase sequence of (from 5′ to 3′) CAAUUUCUGUCUCAUCUUAAA (SEQ ID NO: 1012), wherein each of nucleosides 1, 2, 20 and 21 (from 5′ to 3′) comprises a 2′-MOE modified sugar moiety, each of nucleosides 7, 9, and 11 (from 5′ to 3′) comprises a 2′-fluoro modified sugar moiety, and each of nucleosides 3-6, 8, 10, and 12-19 (from 5′ to 3′) comprises a 2′-OMe modified sugar moiety, wherein each of the internucleoside linkages linking nucleosides 1 to 2, 2 to 3, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, and 18 to 19 (from 5′ to 3′) is a phosphodiester linkage.

The second oligomeric compound is represented by the following chemical notation: CₑₛAₑₛA_{yo}U_{yo}U_{yo}U_{yo}C_{fo}U_{yo}G_{fo}U_{yo}C_{fo}U_{yo}C_{yo}A_{yo}U_{yo}C_{yo}U_{yo}U_{yo}A_{ys}AₑₛAₑ (SEQ ID NO: 1028), wherein
A=an adenine nucleobase,
C=a cytosine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

In particular embodiments, such an oligomeric duplex is represented by the following chemical structure:

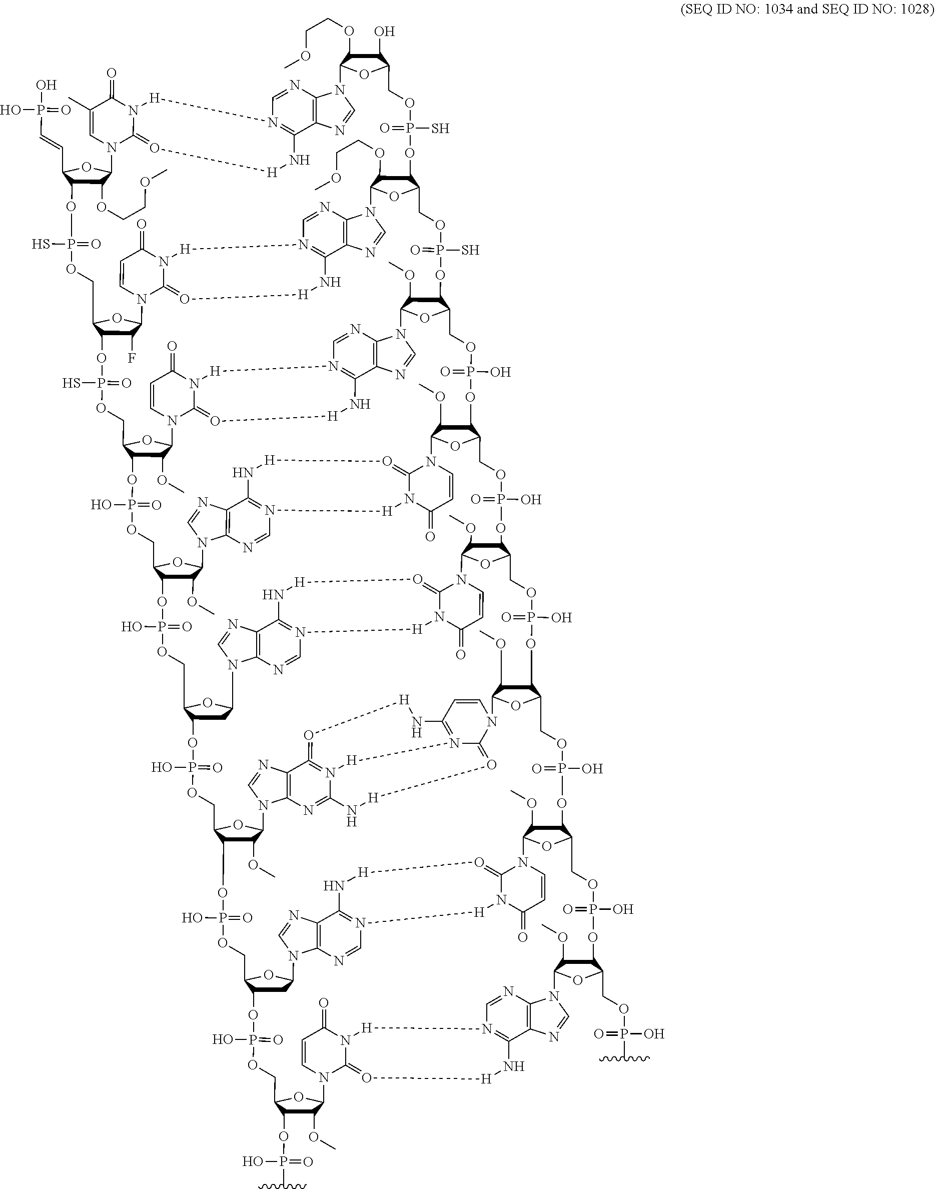

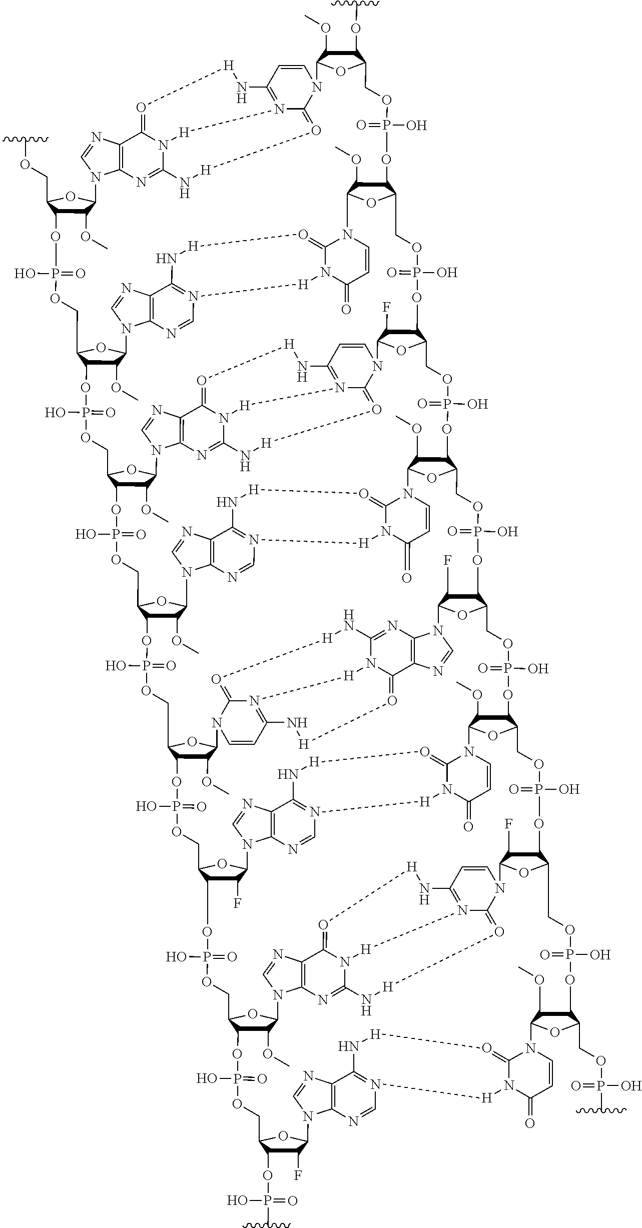

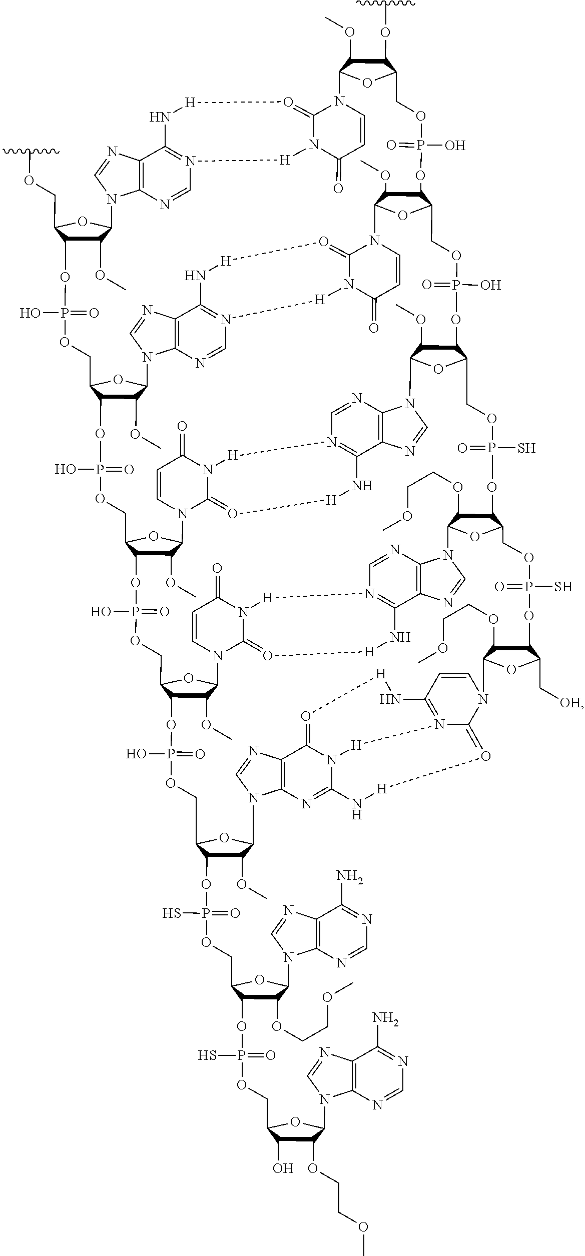

or a salt thereof.

In certain embodiments, such an oligomeric duplex is in the form of an anion or a salt thereof, for example, a sodium salt. In certain embodiments, the oligomeric duplex is in anionic form in a solution. In certain embodiments, the oligomeric duplex is a sodium salt or a potassium salt.

The following chemical structure represents such an oligomeric duplex in sodium solution:

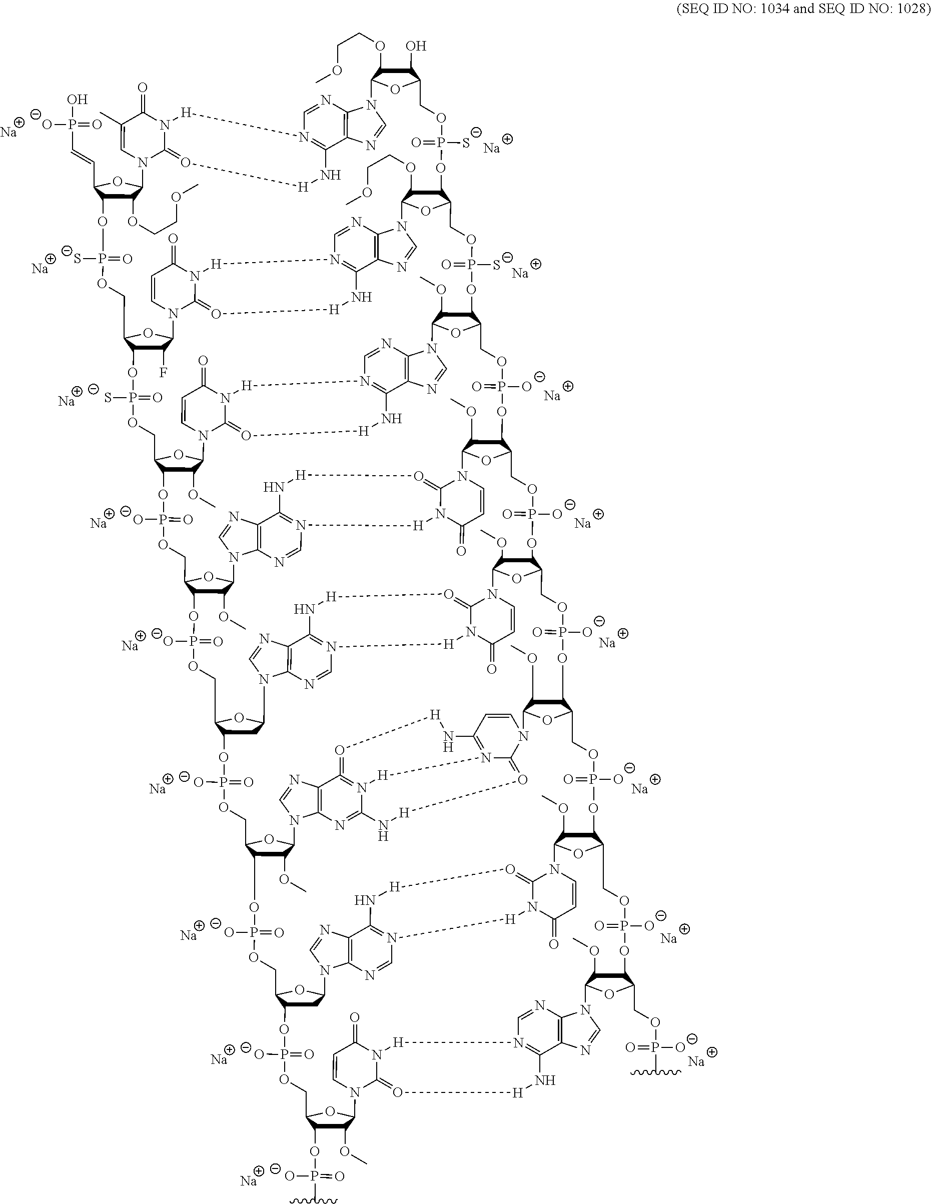

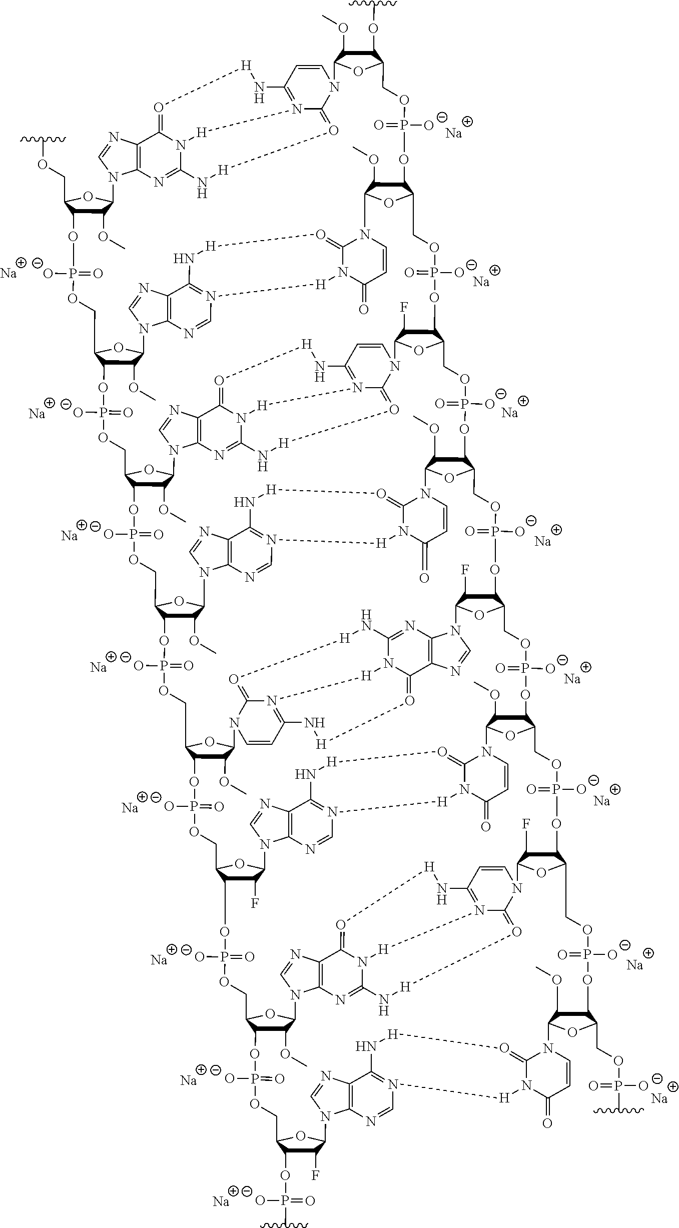

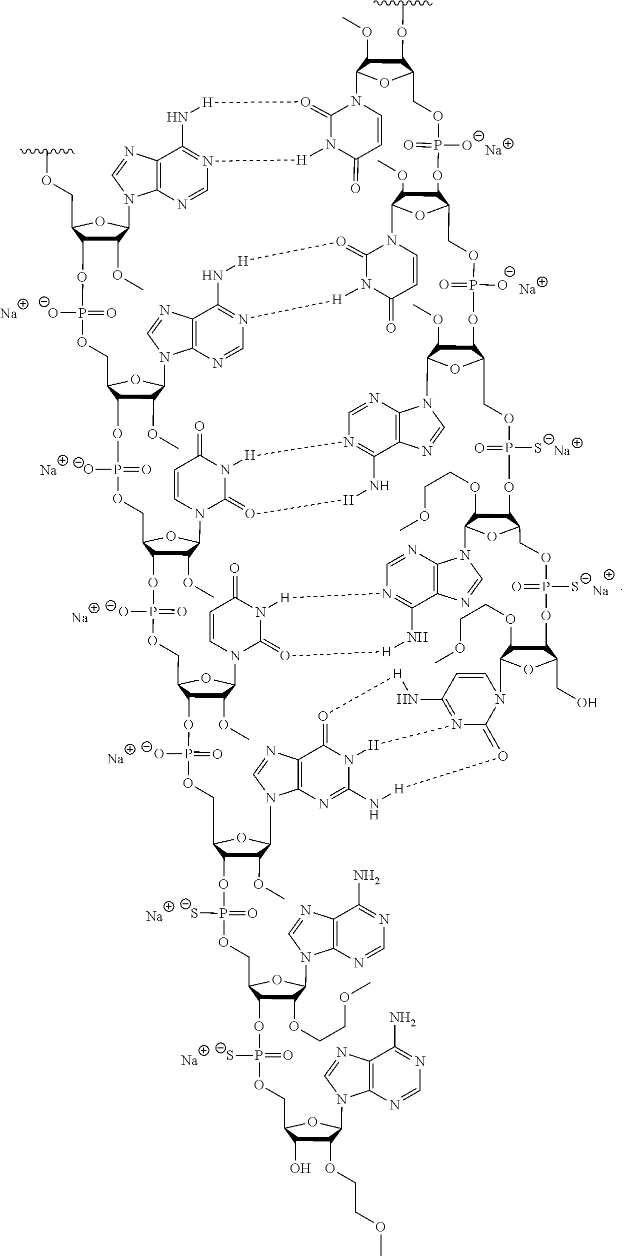

A. c. Copon NoN1577

Provided herein is Compound No. 1757471, which is an oligomeric duplex that consists of a first oligomeric compound containing a first modified oligonucleotide and a second oligomeric compound containing a second modified oligonucleotide attached to a conjugate group as follows.

### First Oligomeric Compound of Compound No. 1757471

The first modified oligonucleotide of Compound no. 1757471, which is Compound No. 1753975, has a nucleobase sequence of (from 5′ to 3′) TAUAAAUAGAUUCUGUAGCUUAA (SEQ ID NO: 974), wherein each of nucleosides 1, 22 and 23 (from 5′ to 3′) comprises a 2′-MOE modified sugar moiety, each of nucleosides 2, 14 and 16 (from 5′ to 3′) comprises a 2′-fluoro modified sugar moiety, each of nucleosides 3-5, 7-13, 15 and 17-21 (from 5′ to 3′) comprises a 2′-OMe modified sugar moiety, and nucleoside 6 (from 5′ to 3′) is a 2′-deoxynucleoside, wherein each of the internucleoside linkages linking nucleosides 1 to 2, 2 to 3, 21 to 22, and 22 to 23 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, 18 to 19, 19 to 20, and 20 to 21 (from 5′ to 3′) is a phosphodiester linkage, and wherein nucleoside 1 (from 5′ to 3′) is attached to a vinyl phosphonate group.

The first oligomeric compound of Compound 1757471 is represented by the following chemical notation: vPTₑₛA_{fs}U_{yo}A_{yo}A_{yo}A_{do}U_{yo}A_{yo}G_{yo}A_{yo}U_{yo}U_{yo}C_{yo}U_{fo}G_{yo}U_{fo}A_{yo}G_{yo}C_{yo}U_{yo}U_{ys}AₑₛAₑ (SEQ ID NO: 1035); wherein
A=an adenine nucleobase,
C=a cytosine nucleobase,
G=a guanine nucleobase,
T=a thymine nucleobase,
U=a uracil nucleobase,
d=a 2′-deoxy sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate group.

### Second Oligomeric Compound of Compound No. 1757471

The second modified oligonucleotide of Compound No. 1757471, which is Compound No. 1757456, has a nucleobase sequence of (from 5′ to 3′) AAGCUACAGAAUCUAUUUATA (SEQ ID NO: 1013), wherein each of nucleosides 1, 2, 20 and 21 (from 5′ to 3′) comprises a 2′-MOE modified sugar moiety, each of nucleosides 10 and 11 (from 5′ to 3′) comprises a 2′-fluoro modified sugar moiety, and each of nucleosides 3-9, and 12-19 (from 5′ to 3′) comprises a 2′-OMe modified sugar moiety, wherein each of the internucleoside linkages linking nucleosides 1 to 2, 2 to 3, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 1, 11 to 12, 12 to 13, 13 to 14, 14 to 15, 15 to 16, 16 to 17, 17 to 18, and 18 to 19 (from 5′ to 3′) is a phosphodiester linkage, and wherein nucleoside 1 (from 5′ to 3′) is attached through a 5′-phosphodiester linkage and a 6-(BCN-carbamate)hexyl conjugate linker to a conjugate moiety comprising the amino acid sequence of bicycle compound BCY17901 (SEQ ID NO: 1045) wherein each cysteine forms a covalent bond with the molecular scaffold 1,1′,1″-(1,3,5-triazinane-1,3,5-triyl)tris(2-bromoethanone) (TATB).

The second oligomeric compound of Compound 1757471 is represented by the following chemical notation: [X]1-AₑₛAₑₛG_{yo}C_{yo}U_{yo}A_{yo}C_{yo}A_{yo}G_{yo}A_{fo}A_{fo}U_{yo}C_{yo}U_{yo}A_{yo}U_{yo}U_{yo}U_{yo}A_{ys}TₑₛAₑ (SEQ ID NO: 1041); wherein:
C=a cytosine nucleobase,
A=an adenine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage,
[X]₁=a conjugate group having the following structure:

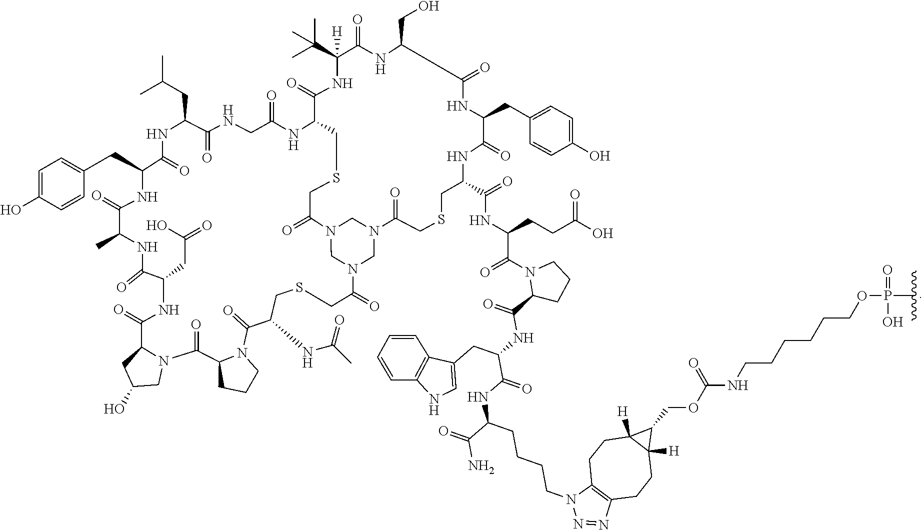 [SMILES: *P(=O)(O)OCCCCCCNC(=O)OC[C@@H]1[C@@H]2CCc3nnn(CCCC[C@H](NC(=O)[C@H](Cc4c[nH]c5ccccc45)NC(=O)[C@@H]4CCCN4C(=O)[C@H](CCC(=O)O)NC(=O)[C@@H]4CSCC(=O)N5CN6CN(C5)C(=O)CSC[C@H](NC(C)=O)C(=O)N5CCC[C@H]5C(=O)N5C[C@H](O)C[C@H]5C(=O)N[C@@H](CC(=O)O)C(=O)N[C@@H](C)C(=O)N[C@@H](Cc5ccc(O)cc5)C(=O)N[C@@H](CC(C)C)C(=O)NCC(=O)N[C@@H](CSCC6=O)C(=O)N[C@@H](C(C)(C)C)C(=O)N[C@@H](CO)C(=O)N[C@@H](Cc5ccc(O)cc5)C(=O)N4)C(N)=O)c3CC[C@@H]21]

The following chemical structure is one structural representation of Compound No. 1757471:

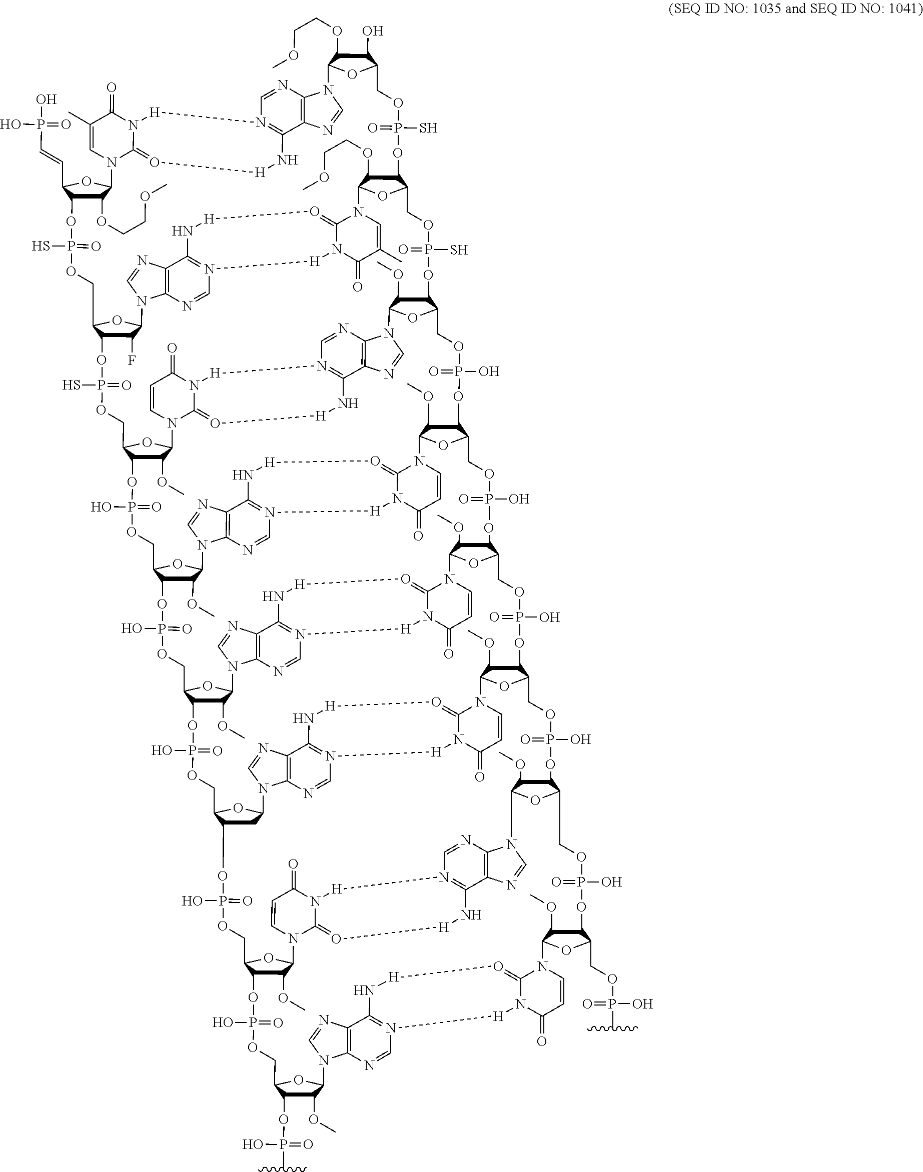

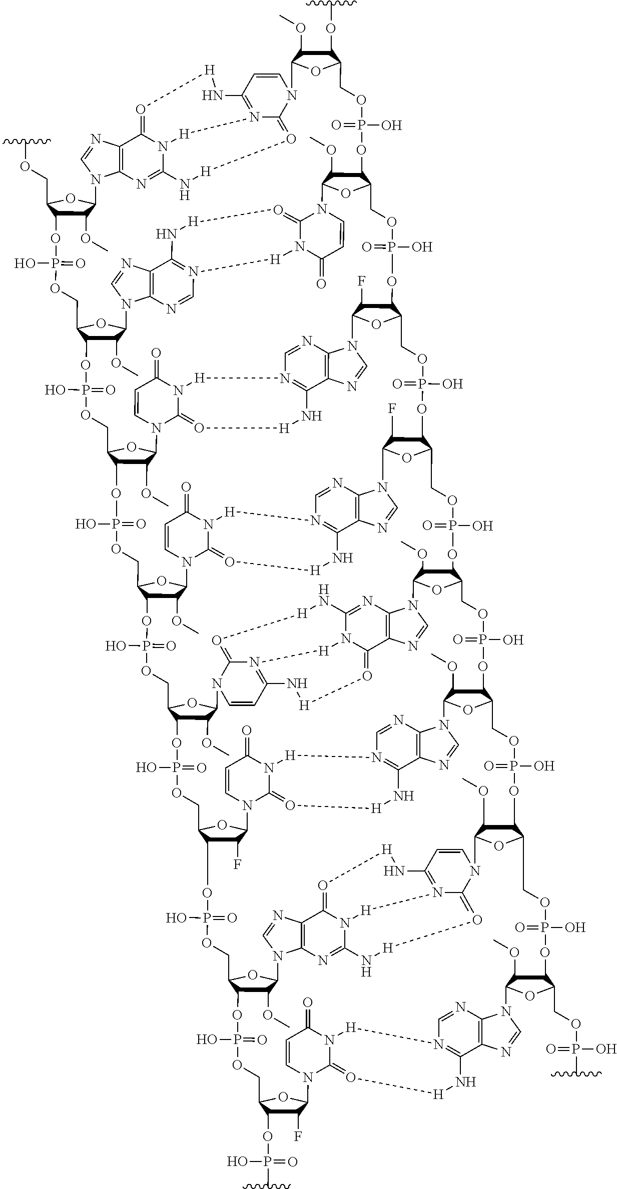

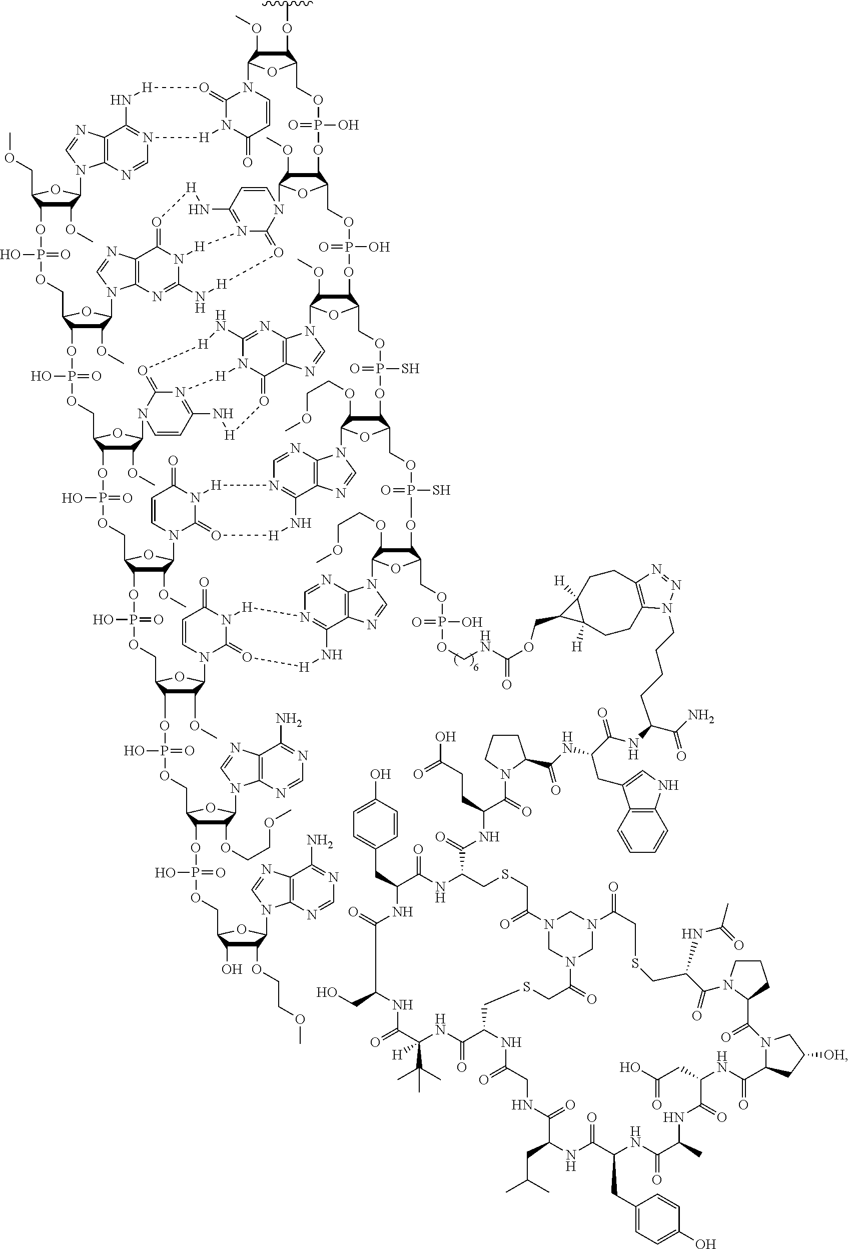

or a salt thereof.

In certain embodiments, Compound No. 1757471 is in the form of an anion or a salt thereof, for example, the oligomeric duplex may be in the form of a sodium salt. In certain embodiments, the oligomeric duplex is in anionic form in a solution. In certain embodiments, Compound No. 1757471 is a sodium salt or a potassium salt.

The following chemical structure represents Compound No. 1757471 in sodium solution:

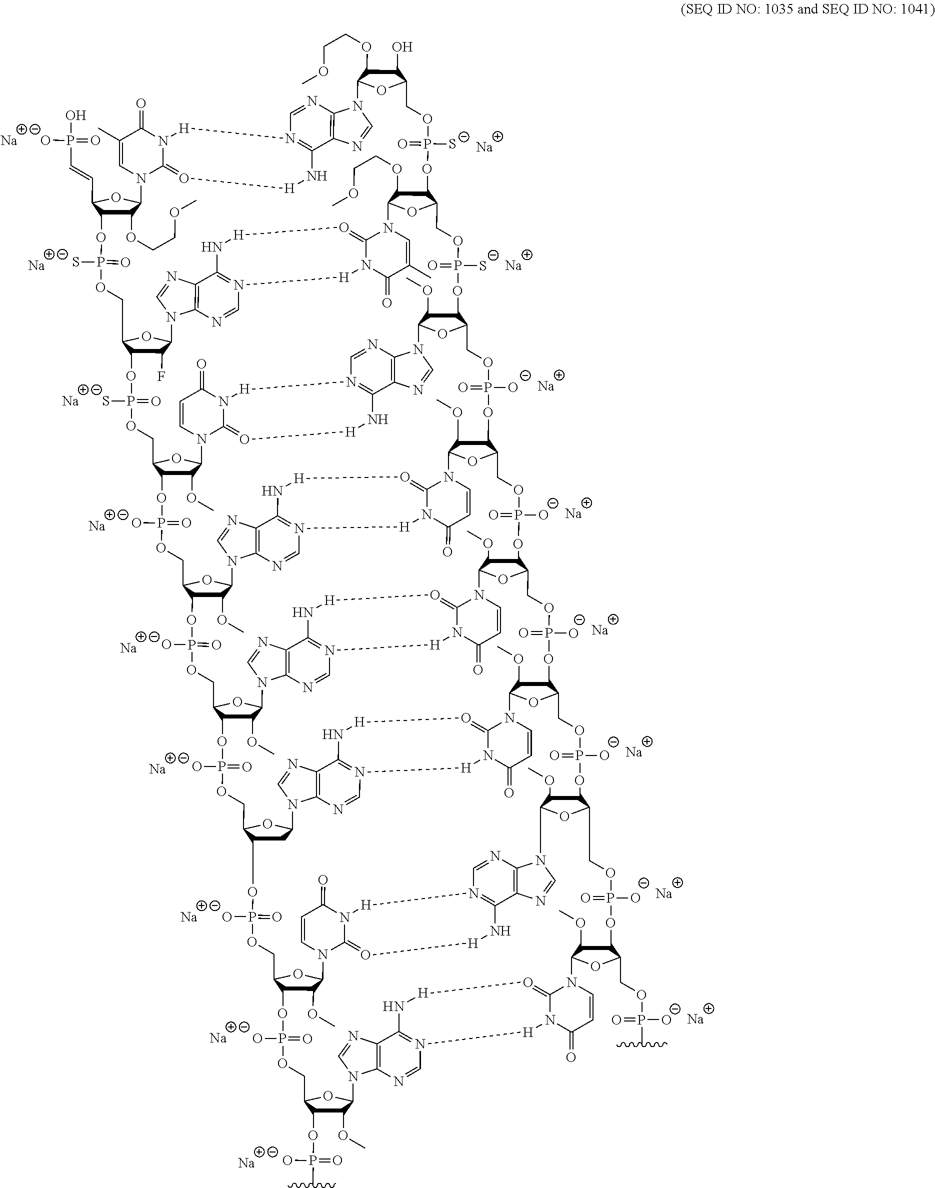

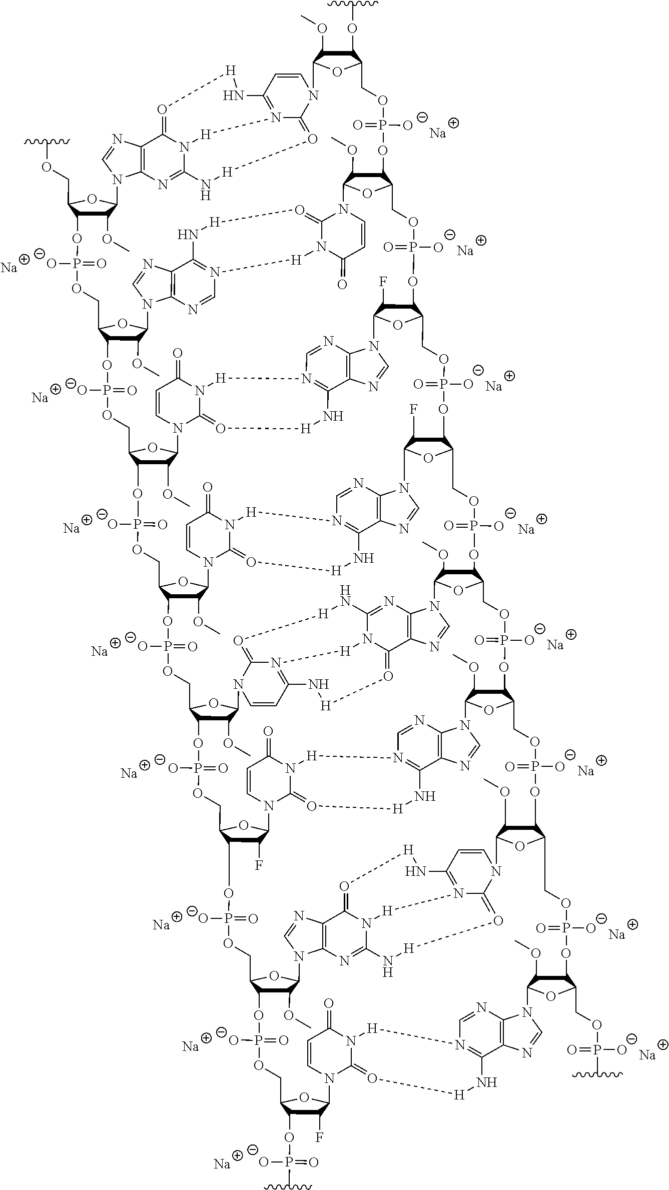

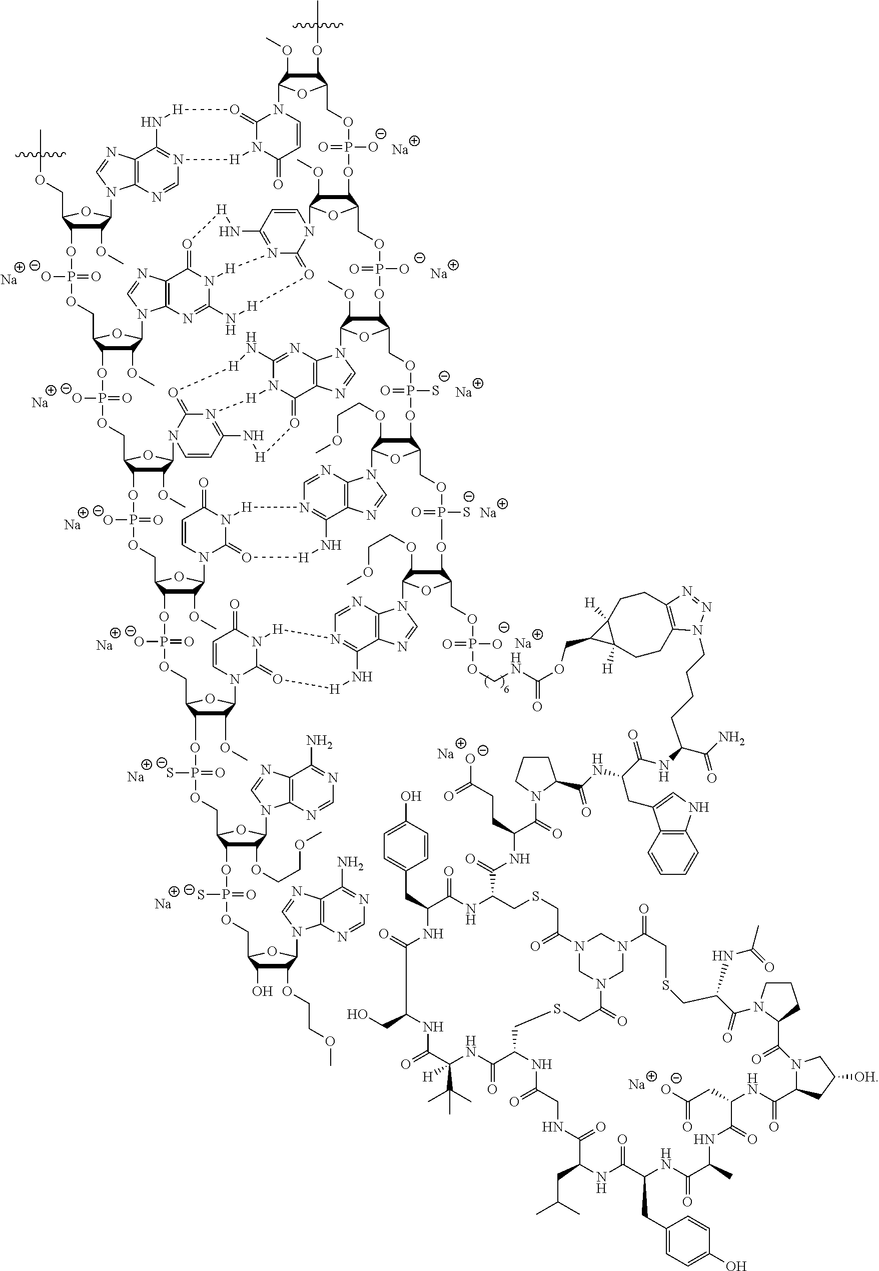

In certain embodiments, provided herein are oligomeric duplexes comprising or consisting of a first oligomeric compound containing a first modified oligonucleotide and a second oligomeric compound containing a second modified oligonucleotide as follows.

### First Oligomeric Compound

The first modified oligonucleotide has a nucleobase sequence of (from 5′ to 3′) TAUAAAUAGAUUCUGUAGCUUAA (SEQ ID NO: 974), wherein each of nucleosides 1, 22 and 23 (from 5′ to 3′) comprises a 2′-MOE modified sugar moiety, each of nucleosides 2, 14 and 16 (from 5′ to 3′) comprises a 2′-fluoro modified sugar moiety, each of nucleosides 3-5, 7-13, 15 and 17-21 (from 5′ to 3′) comprises a 2′-OMe modified sugar moiety, and nucleoside 6 (from 5′ to 3′) is a 2′-deoxynucleoside, wherein each of the internucleoside linkages linking nucleosides 1 to 2, 2 to 3, 21 to 22, and 22 to 23 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, 18 to 19, 19 to 20, and 20 to 21 (from 5′ to 3′) is a phosphodiester linkage, and wherein nucleoside 1 (from 5′ to 3′) is attached to a vinyl phosphonate group.

The first oligomeric compound is represented by the following chemical notation:

(SEQ ID NO: 1035)
vPTₑₛA_{fs}U_{yo}A_{yo}A_{yo}A_{do}U_{yo}A_{yo}G_{yo}A_{yo}U_{yo}U_{yo}C_{yo}U_{fo}G_{yo}U_{fo}
A_{yo}G_{yo}C_{yo}U_{yo}U_{ys}AₑₛAₑ;

wherein
A=an adenine nucleobase,
C=a cytosine nucleobase,
G=a guanine nucleobase,
T=a thymine nucleobase,
U=a uracil nucleobase,
d=a 2′-deoxy sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate group.

### Second Oligomeric Compound

The second modified oligonucleotide has a nucleobase sequence of (from 5′ to 3′) AAGCUACAGAAUCUAUUUATA (SEQ ID NO: 1013), wherein each of nucleosides 1, 2, 20 and 21 (from 5′ to 3′) comprises a 2′-MOE modified sugar moiety, each of nucleosides 10 and 11 (from 5′ to 3′) comprises a 2′-fluoro modified sugar moiety, and each of nucleosides 3-9, and 12-19 (from 5′ to 3′) comprises a 2′-OMe modified sugar moiety, wherein each of the internucleoside linkages linking nucleosides 1 to 2, 2 to 3, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, and 18 to 19 (from 5′ to 3′) is a phosphodiester linkage.

The second oligomeric compound is represented by the following chemical notation:

(SEQ ID NO: 1029)
AₑₛAₑₛG_{yo}C_{yo}U_{yo}A_{yo}C_{yo}A_{yo}G_{yo}A_{fo}A_{fo}U_{yo}C_{yo}U_{yo}A_{yo}U_{yo}
U_{yo}U_{yo}A_{ys}TₑₛAₑ;

A=an adenine nucleobase,
C=a cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

In particular embodiments, such an oligomeric duplex is represented by the following chemical structure:

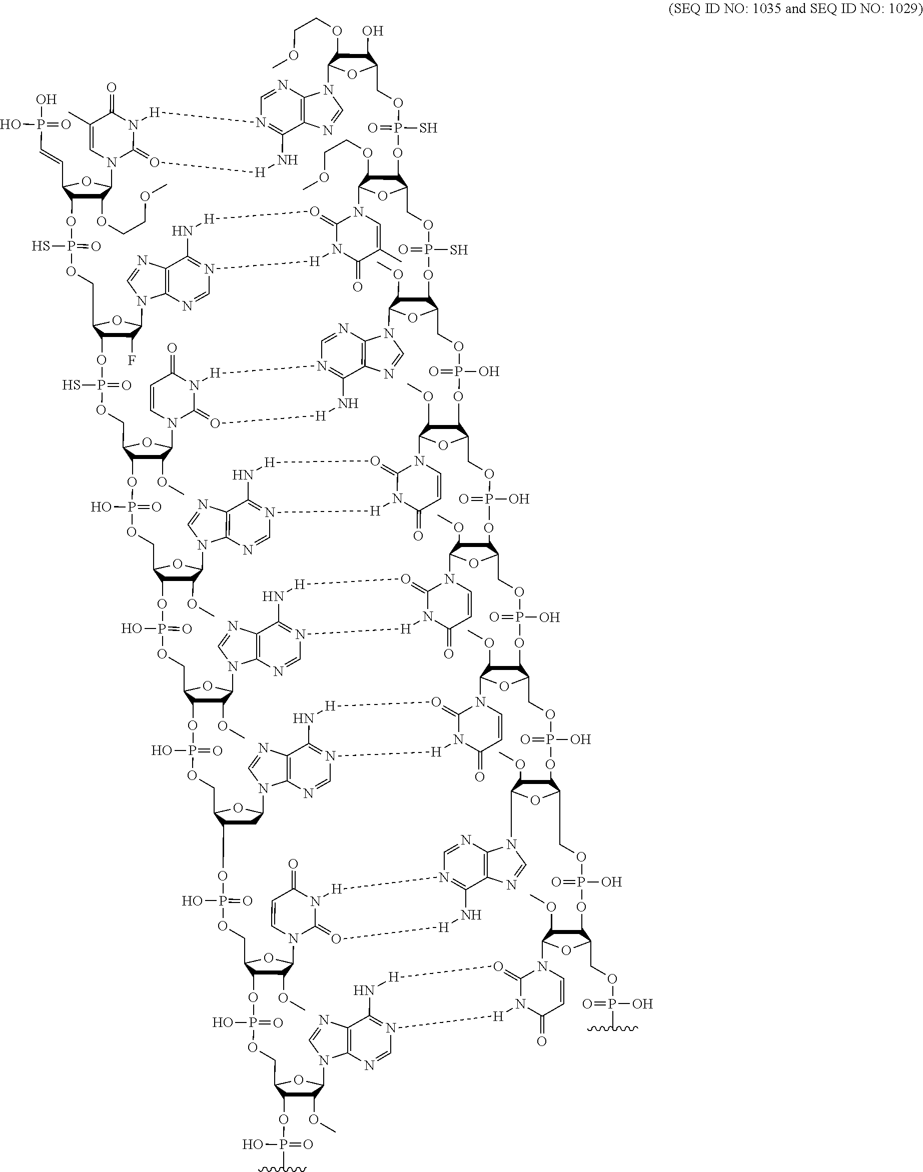

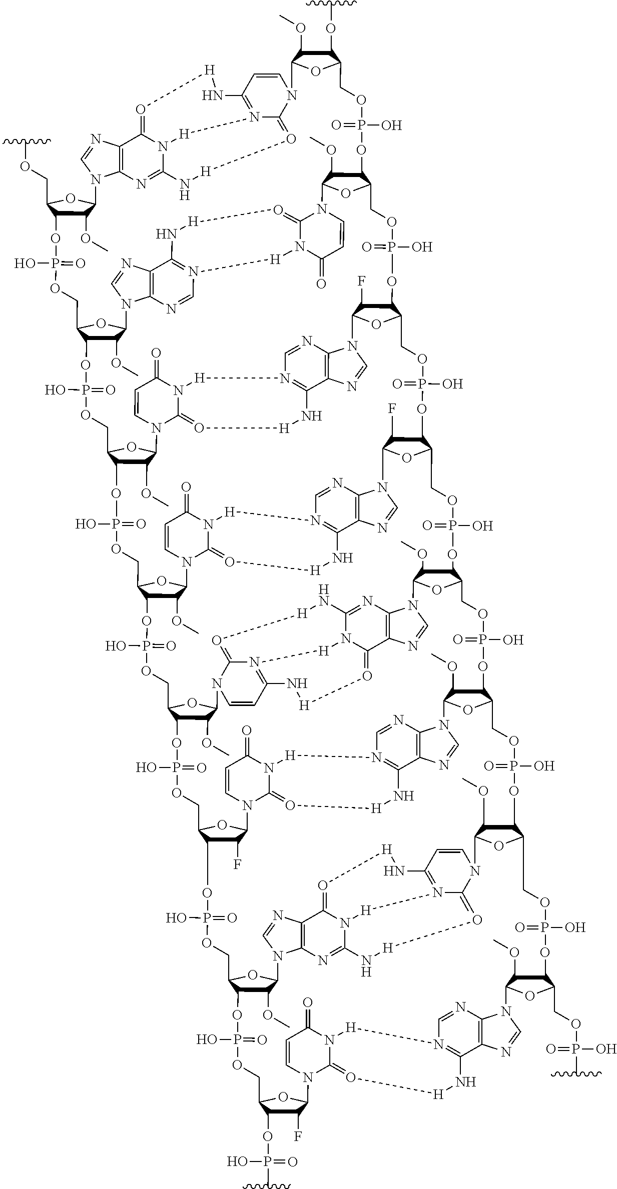

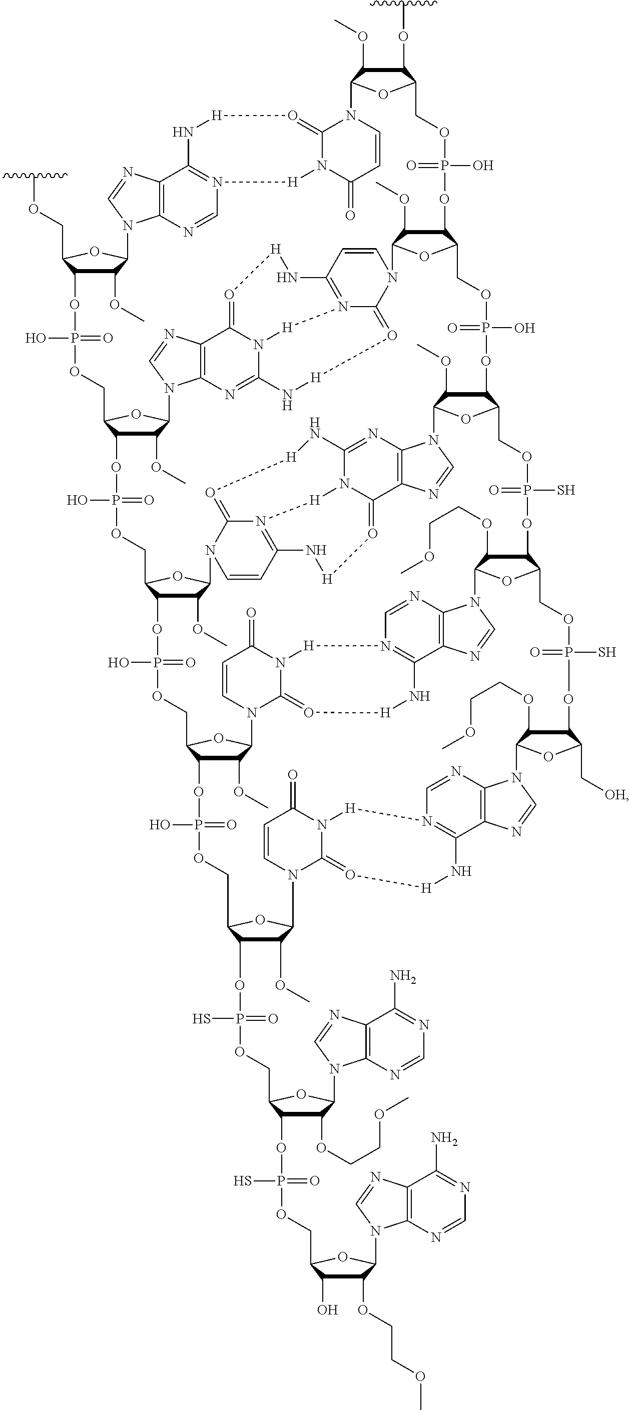

or a salt thereof.

In certain embodiments, such an oligomeric duplex is in the form of an anion or a salt thereof, for example, a sodium salt. In certain embodiments, the oligomeric duplex is in anionic form in a solution. In certain embodiments, the oligomeric duplex is a sodium salt or a potassium salt.

The following chemical structure represents such an oligomeric duplex in sodium solution:

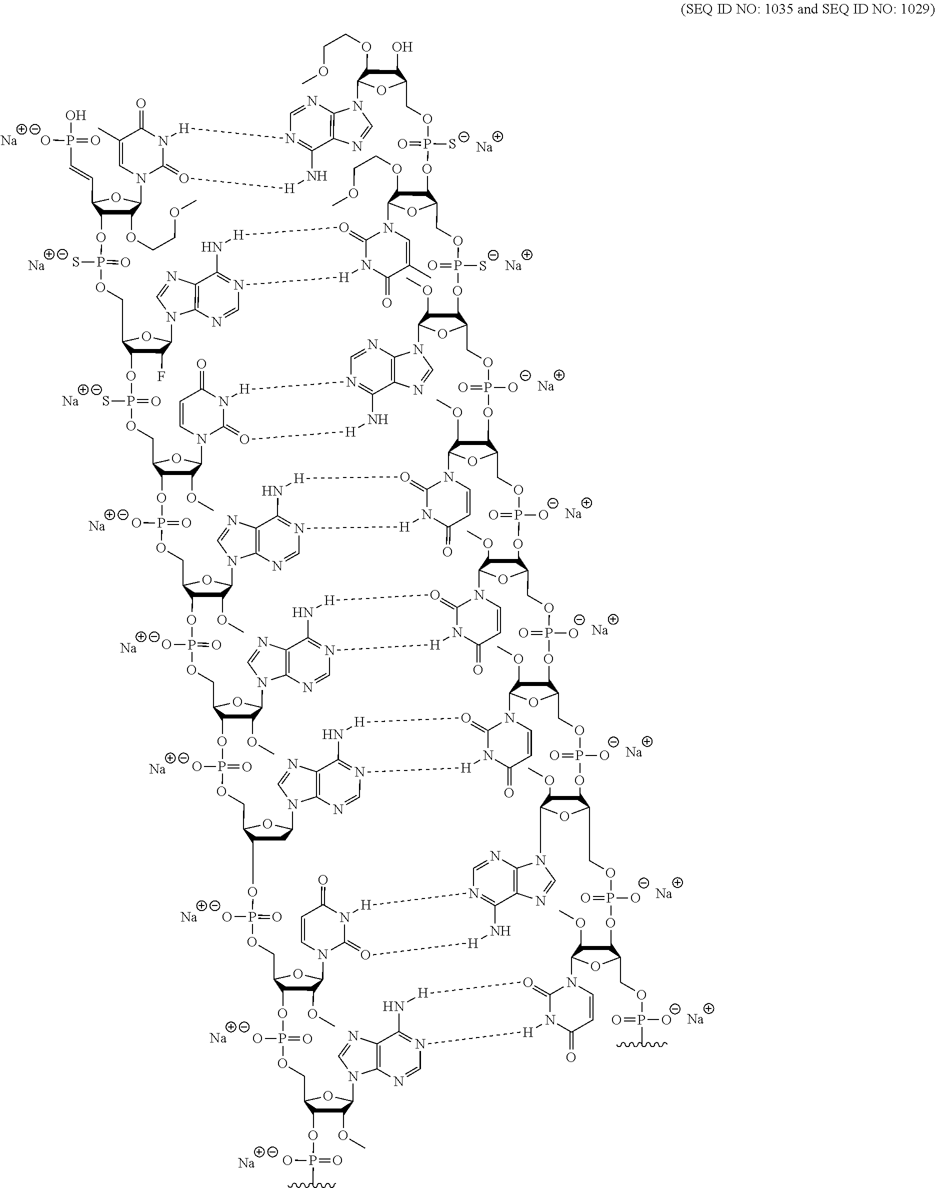

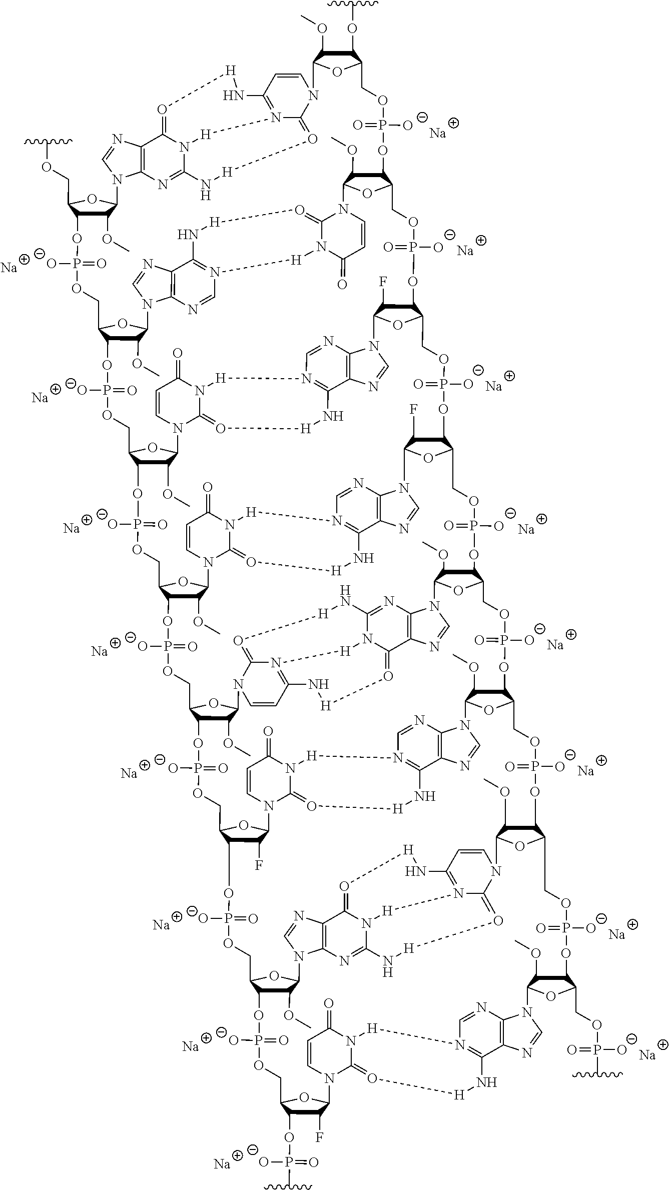

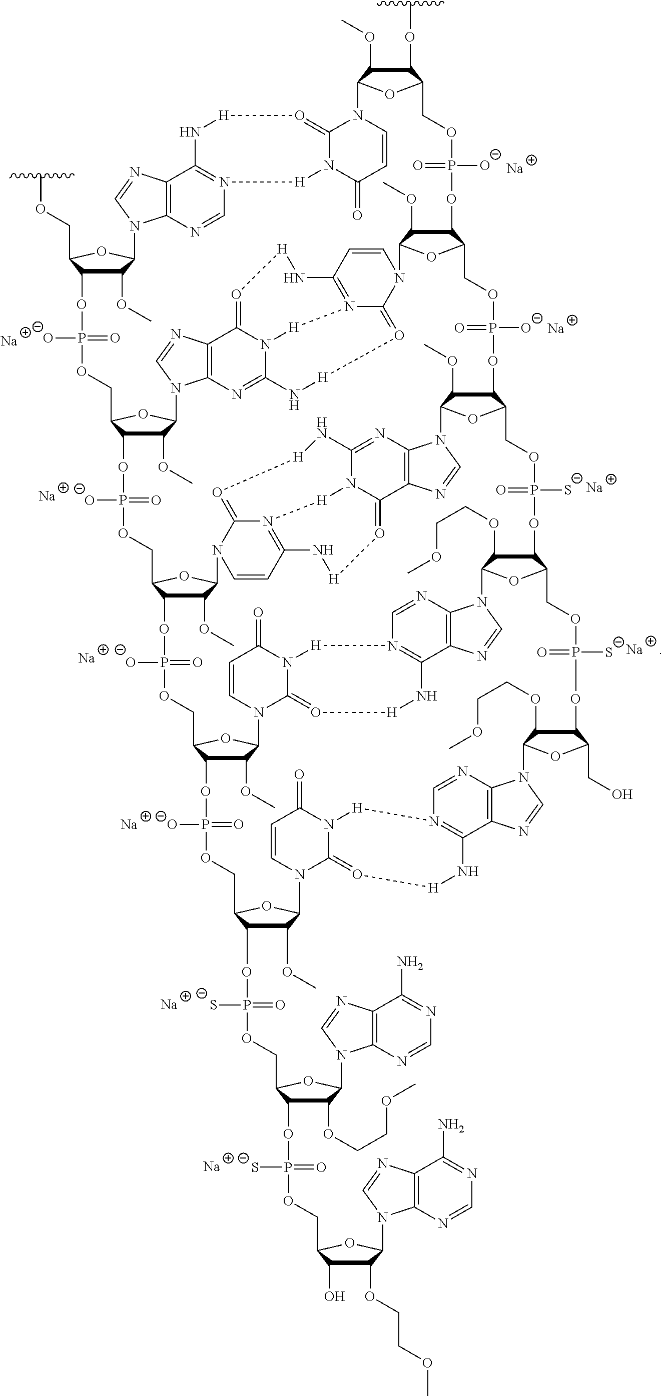

d. Compound No. 1757474

Provided herein is Compound No. 1757474, which is an oligomeric duplex that consists of a first oligomeric compound containing a first modified oligonucleotide and a second oligomeric compound containing a second modified oligonucleotide attached to a conjugate group as follows.

### First Oligomeric Compound of Compound No. 1757474

The first modified oligonucleotide of Compound No. 1757474, which is Compound No. 1753976, has a nucleobase sequence of (from 5′ to 3′) TAUAAATAGAUUCUGUAGCUUAA (SEQ ID NO: 975), wherein each of nucleosides 1, 22 and 23 (from 5′ to 3′) comprises a 2′-MOE modified sugar moiety, each of nucleosides 2, 14 and 16 (from 5′ to 3′) comprises a 2′-fluoro modified sugar moiety, each of nucleosides 3, 4, 6, 8-13, 15 and 17-21 (from 5′ to 3′) comprises a 2′-OMe modified sugar moiety, and each of nucleosides 5 and 7 (from 5′ to 3′) is a 2′-deoxynucleoside, wherein each of the internucleoside linkages linking nucleosides 1 to 2, 2 to 3, 21 to 22, and 22 to 23 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, 18 to 19, 19 to 20, and 20 to 21 (from 5′ to 3′) is a phosphodiester linkage, and wherein nucleoside 1 (from 5′ to 3′) is attached to a vinyl phosphonate group.

The first oligomeric compound of Compound 1757474 is represented by the following chemical notation: vPTₑₛA_{fs}U_{yo}A_{yo}A_{do}A_{yo}T_{do}A_{yo}G_{yo}A_{yo}U_{yo}U_{yo}C_{yo}U_{fo}G_{yo}U_{fo}A_{yo}G_{yo}C_{yo}U_{yo}U_{ys}AₑₛAₑ (SEQ ID NO: 1036); wherein
A=an adenine nucleobase,
C=a cytosine nucleobase,
G=a guanine nucleobase,
T=a thymine nucleobase,
U=a uracil nucleobase,
d=a 2′-deoxy sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate group.

### Second Oligomeric Compound of Compound No. 1757474

The second modified oligonucleotide of Compound No. 1757474, which is Compound No. 1757458, has a nucleobase sequence of (from 5′ to 3′) AAGCUACAGAAUCUAUUUAUA (SEQ ID NO: 1014), wherein each of nucleosides 1, 2, 20 and 21 (from 5′ to 3′) comprises a 2′-MOE modified sugar moiety, each of nucleosides 7, 9, and 11 (from 5′ to 3′) comprises a 2′-fluoro modified sugar moiety, and each of nucleosides 3-6, 8, 10, and 12-19 (from 5′ to 3′) comprises a 2′-OMe modified sugar moiety, wherein each of the internucleoside linkages linking nucleosides 1 to 2, 2 to 3, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, and 18 to 19 (from 5′ to 3′) is a phosphodiester linkage, and wherein nucleoside 1 (from 5′ to 3′) is attached through a 5′-phosphodiester linkage and a 6-(BCN-carbamate)hexyl conjugate linker to a conjugate moiety comprising the amino acid sequence of bicycle compound BCY17901 (SEQ ID NO: 1045) wherein each cysteine forms a covalent bond with the molecular scaffold 1,1′,1″-(1,3,5-triazinane-1,3,5-triyl) tris(2-bromoethanone) (TATB).

The second oligomeric compound of Compound 1757474 is represented by the following chemical notation: [X]₁-AₑₛAₑₛG_{yo}C_{yo}U_{yo}A_{yo}C_{fo}A_{yo}G_{fo}A_{yo}A_{fo}U_{yo}C_{yo}U_{yo}A_{yo}U_{yo}U_{yo}U_{yo}A_{ys}UₑₛAₑ (SEQ ID NO: 1042); wherein:
C=a cytosine nucleobase,
A=an adenine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
[X]₁=a conjugate group having the following structure:

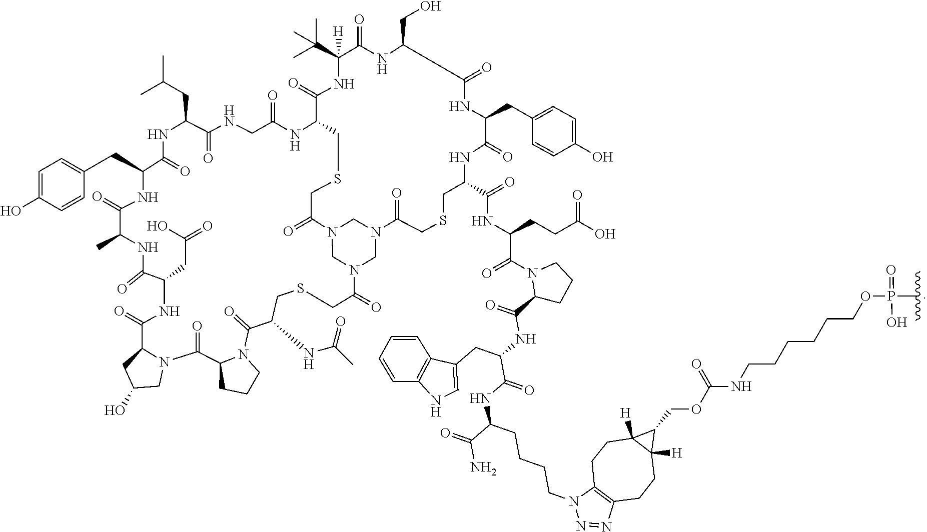 [SMILES: *P(=O)(O)OCCCCCCNC(=O)OC[C@@H]1[C@@H]2CCc3nnn(CCCC[C@H](NC(=O)[C@H](Cc4c[nH]c5ccccc45)NC(=O)[C@@H]4CCCN4C(=O)[C@H](CCC(=O)O)NC(=O)[C@@H]4CSCC(=O)N5CN6CN(C5)C(=O)CSC[C@H](NC(C)=O)C(=O)N5CCC[C@H]5C(=O)N5C[C@H](O)C[C@H]5C(=O)N[C@@H](CC(=O)O)C(=O)N[C@@H](C)C(=O)N[C@@H](Cc5ccc(O)cc5)C(=O)N[C@@H](CC(C)C)C(=O)NCC(=O)N[C@@H](CSCC6=O)C(=O)N[C@@H](C(C)(C)C)C(=O)N[C@@H](CO)C(=O)N[C@@H](Cc5ccc(O)cc5)C(=O)N4)C(N)=O)c3CC[C@@H]21]

The following chemical structure is one structural representation of Compound No. 1757474:

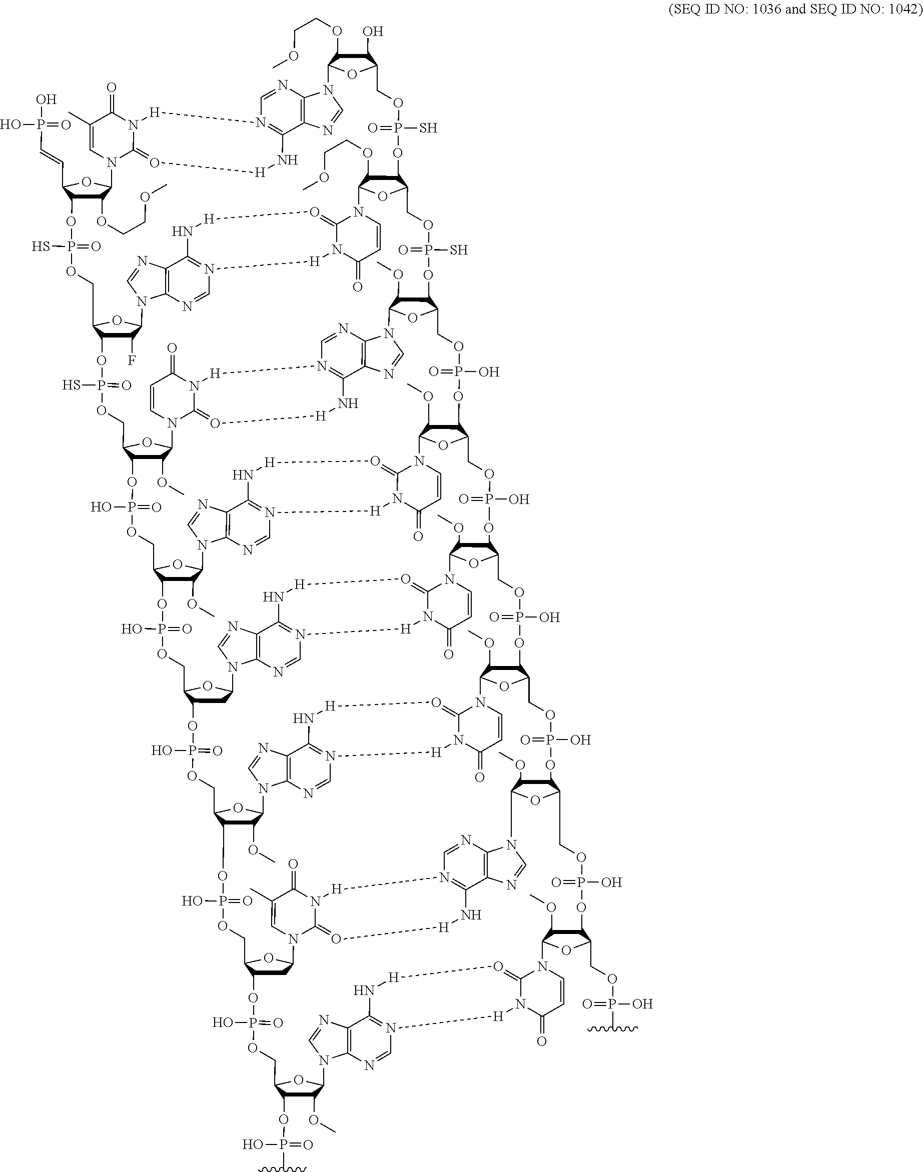

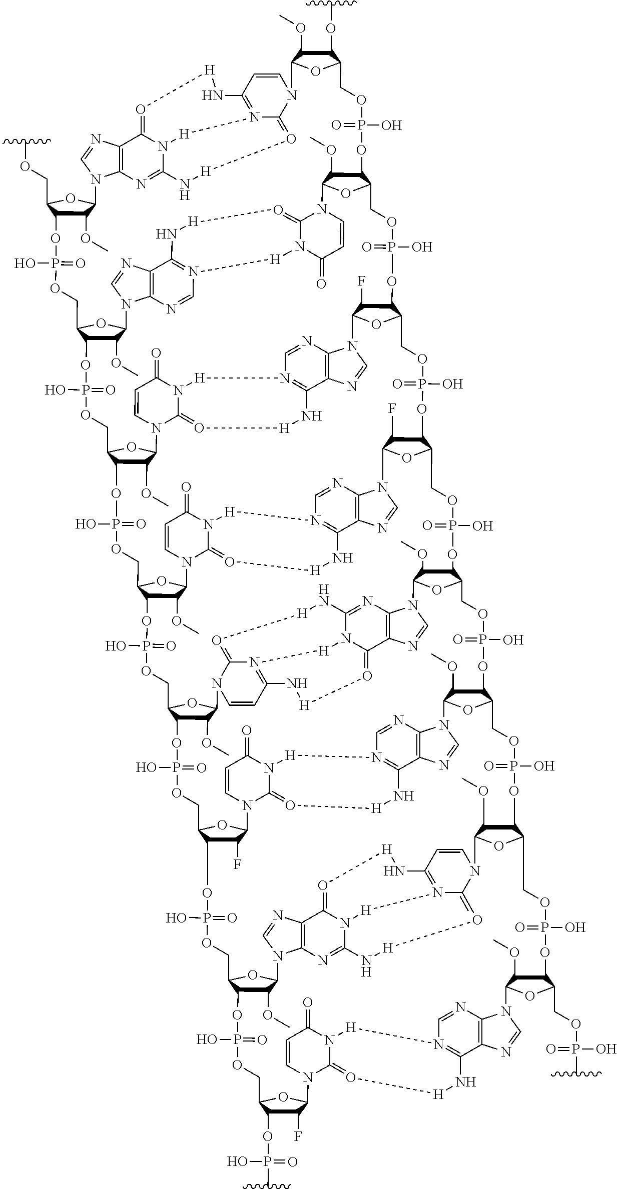

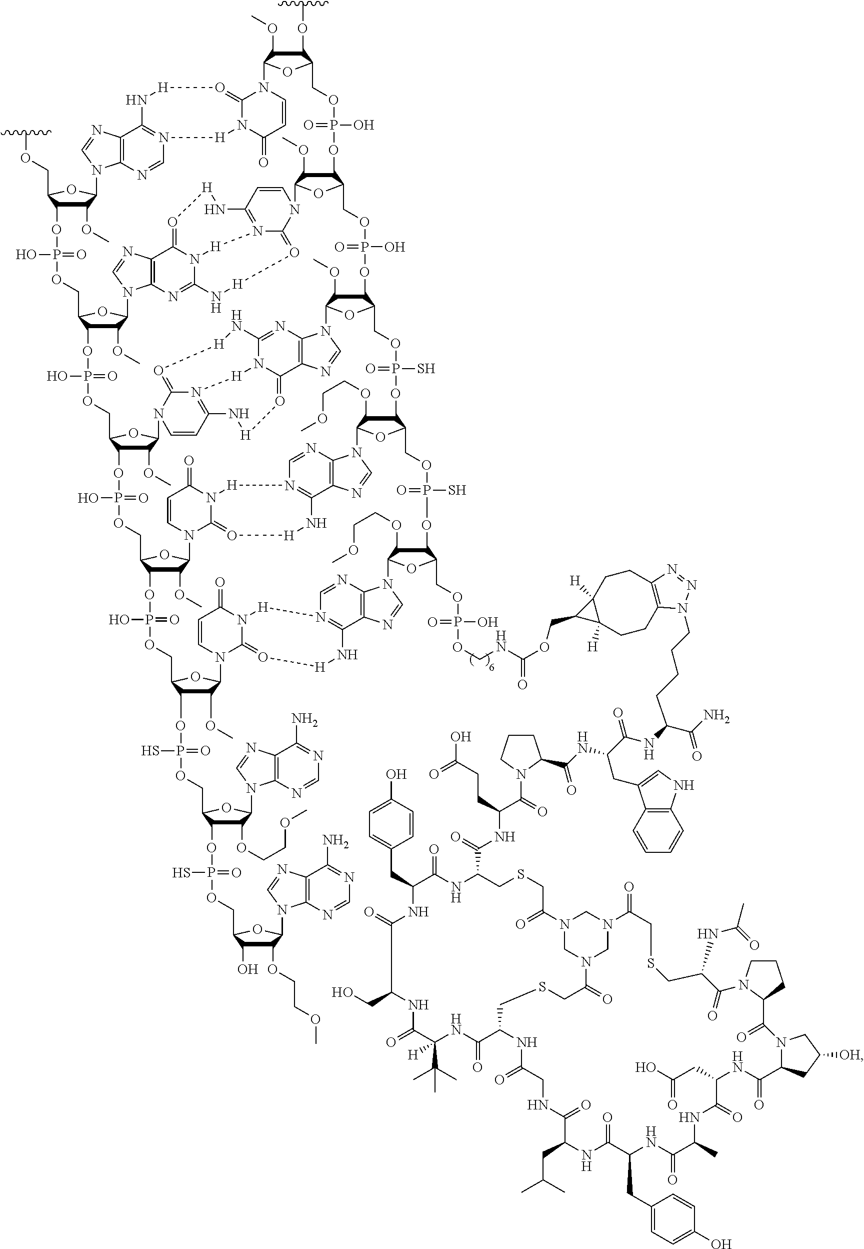

or a salt thereof.

In certain embodiments, Compound No. 1757474 is in the form of an anion or a salt thereof, for example, a sodium salt. In certain embodiments, the oligomeric duplex is in anionic form in a solution. In certain embodiments, Compound No. 1757474 is a sodium salt or a potassium salt.

The following chemical structure represents Compound No. 1757474 in sodium solution:

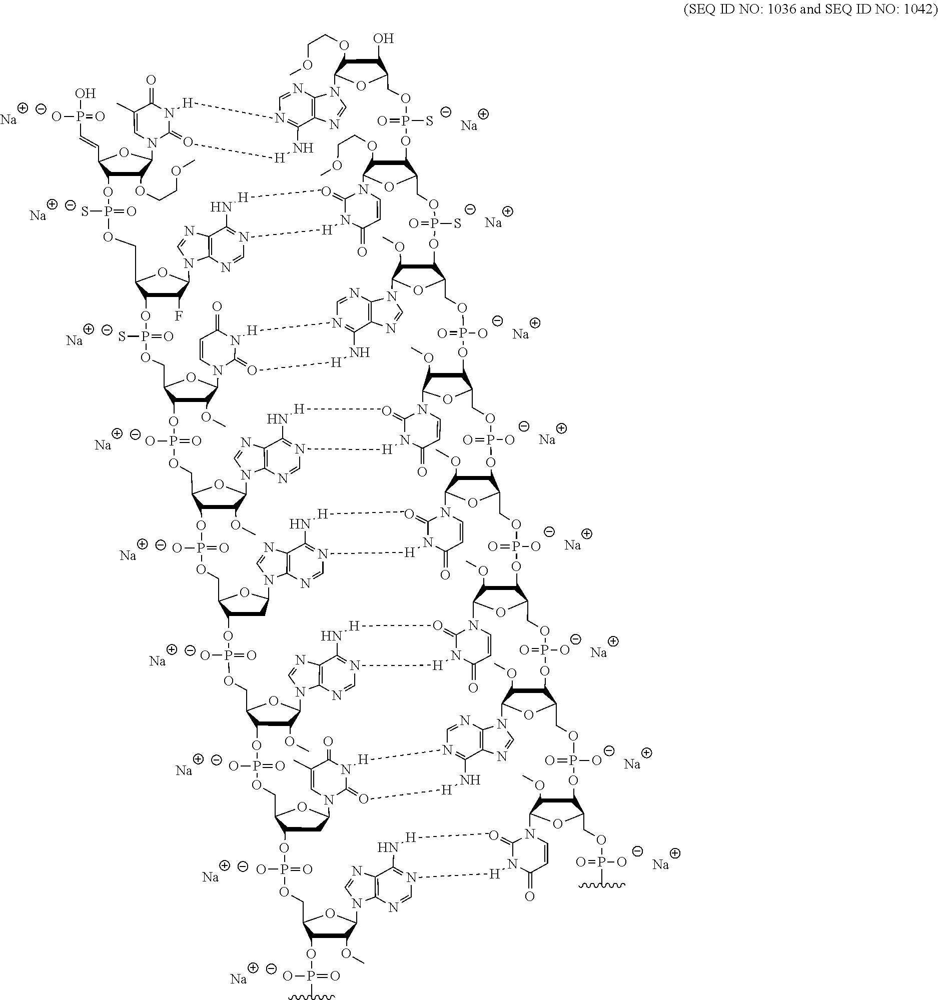

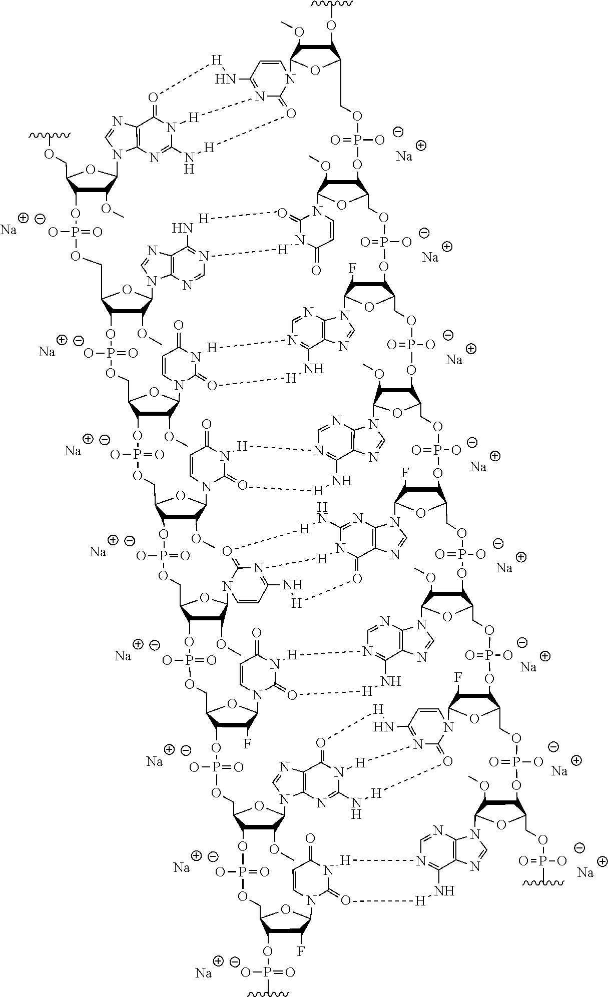

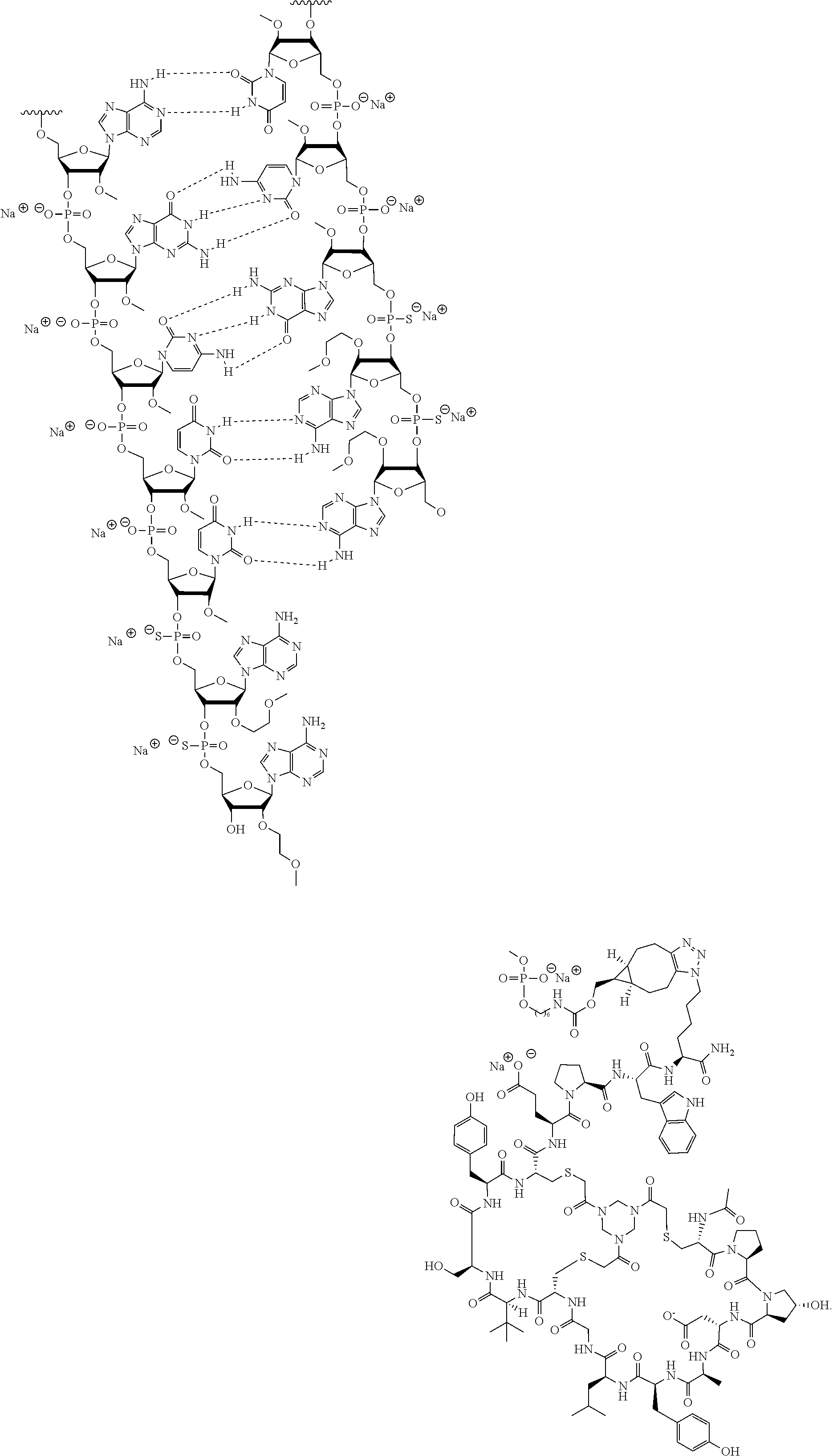

In certain embodiments, provided herein are oligomeric duplexes comprising or consisting of a first oligomeric compound containing a first modified oligonucleotide and a second oligomeric compound containing a second modified oligonucleotide as follows.

### First Oligomeric Compound

The first modified oligonucleotide has a nucleobase sequence of (from 5′ to 3′) TAUAAATAGAUUCUGUAGCUUAA (SEQ ID NO: 975), wherein each of nucleosides 1, 22 and 23 (from 5′ to 3′) comprises a 2′-MOE modified sugar moiety, each of nucleosides 2, 14 and 16 (from 5′ to 3′) comprises a 2′-fluoro modified sugar moiety, each of nucleosides 3, 4, 6, 8-13, 15 and 17-21 (from 5′ to 3′) comprises a 2′-OMe modified sugar moiety, and each of nucleosides 5 and 7 (from 5′ to 3′) is a 2′-deoxynucleoside, wherein each of the internucleoside linkages linking nucleosides 1 to 2, 2 to 3, 21 to 22, and 22 to 23 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, 18 to 19, 19 to 20, and 20 to 21 (from 5′ to 3′) is a phosphodiester linkage, and wherein nucleoside 1 (from 5′ to 3′) is attached to a vinyl phosphonate group.

The first oligomeric compound is represented by the following chemical notation:

(SEQ ID NO: 1036)
vPTₑₛA_{fs}U_{yo}A_{yo}A_{do}A_{yo}T_{do}A_{yo}G_{yo}A_{yo}U_{yo}U_{yo}C_{yo}U_{fo}G_{yo}U_{fo}
A_{yo}G_{yo}C_{yo}U_{yo}U_{ys}AₑₛAₑ;

A=an adenine nucleobase,
C=a cytosine nucleobase,
G=a guanine nucleobase,
T=a thymine nucleobase,
U=a uracil nucleobase,
d=a 2′-deoxy sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate group.

### Second Oligomeric Compound

The second modified oligonucleotide has a nucleobase sequence of (from 5′ to 3′) AAGCUACAGAAUCUAUUUAUA (SEQ ID NO: 1014), wherein each of nucleosides 1, 2, 20 and 21 (from 5′ to 3′) comprises a 2′-MOE modified sugar moiety, each of nucleosides 7, 9, and 11 (from 5′ to 3′) comprises a 2′-fluoro modified sugar moiety, and each of nucleosides 3-6, 8, 10, and 12-19 (from 5′ to 3′) comprises a 2′-OMe modified sugar moiety, wherein each of the internucleoside linkages linking nucleosides 1 to 2, 2 to 3, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, and 18 to 19 (from 5′ to 3′) is a phosphodiester linkage.

The second oligomeric compound is represented by the following chemical notation:

(SEQ ID NO: 1030)
AₑₛAₑₛG_{yo}C_{yo}U_{yo}A_{yo}C_{fo}A_{yo}G_{fo}A_{yo}A_{fo}U_{yo}C_{yo}U_{yo}A_{yo}U_{yo}
U_{yo}U_{yo}A_{ys}UₑₛAₑ,

wherein
A=an adenine nucleobase,
C=a cytosine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

In particular embodiments, such an oligomeric duplex is represented by the following chemical structure:

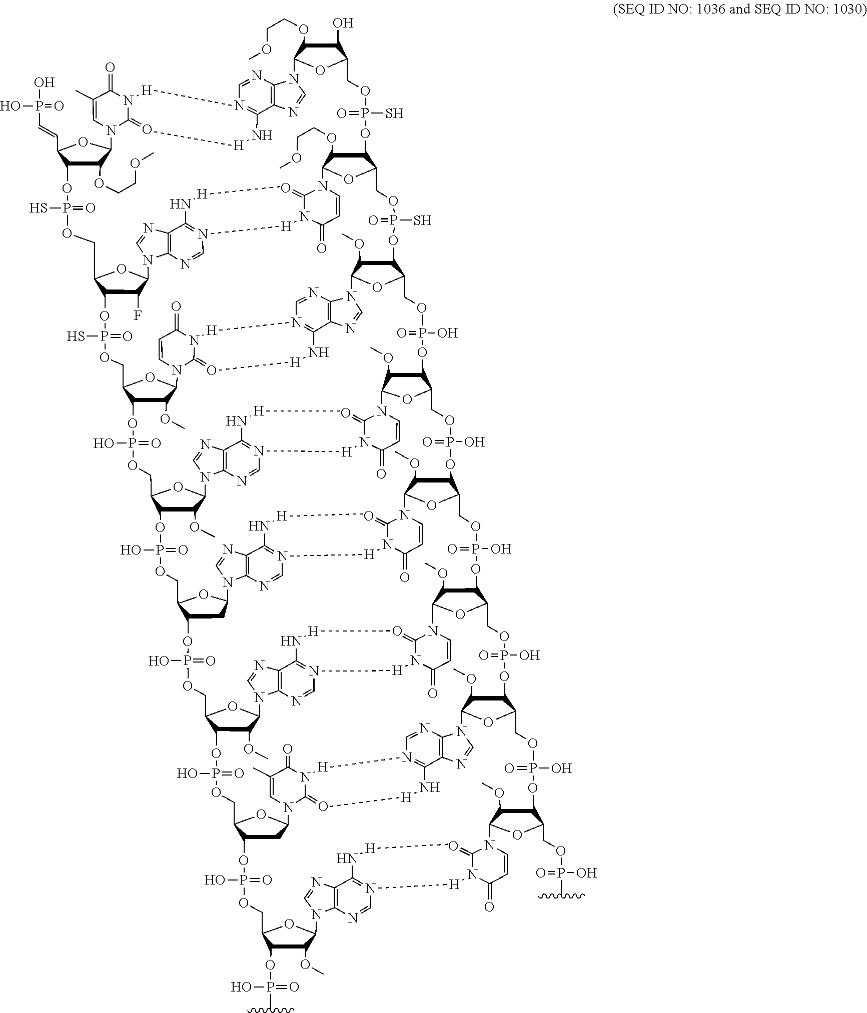

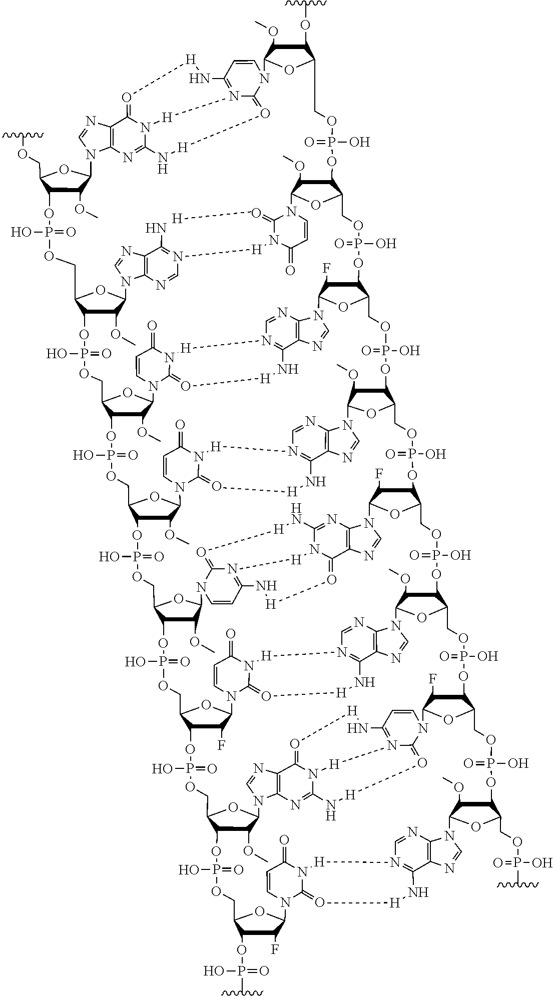

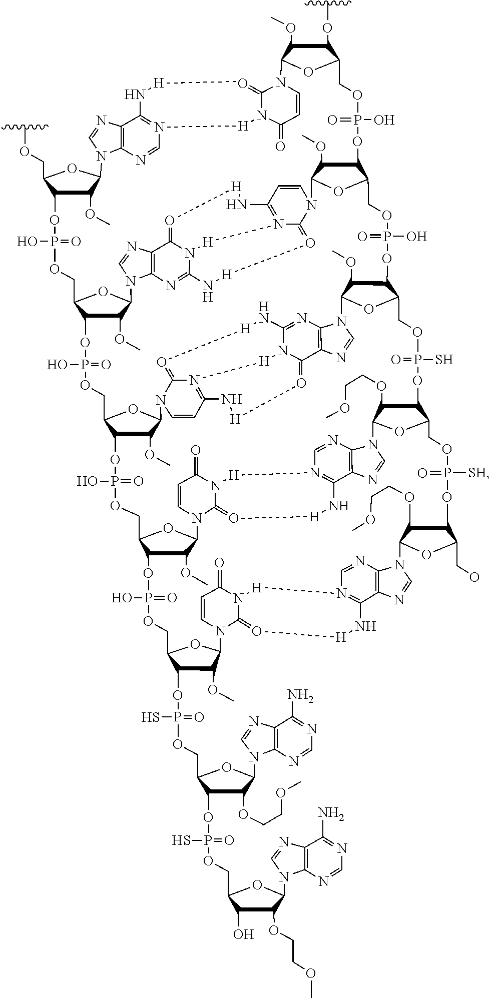

or a salt thereof.

In certain embodiments, such an oligomeric duplex is in the form of an anion or a salt thereof, for example, a sodium salt. In certain embodiments, the oligomeric duplex is in anionic form in a solution. In certain embodiments, the oligomeric duplex is a sodium salt or a potassium salt.

The following chemical structure represents such an oligomeric duplex in sodium solution:

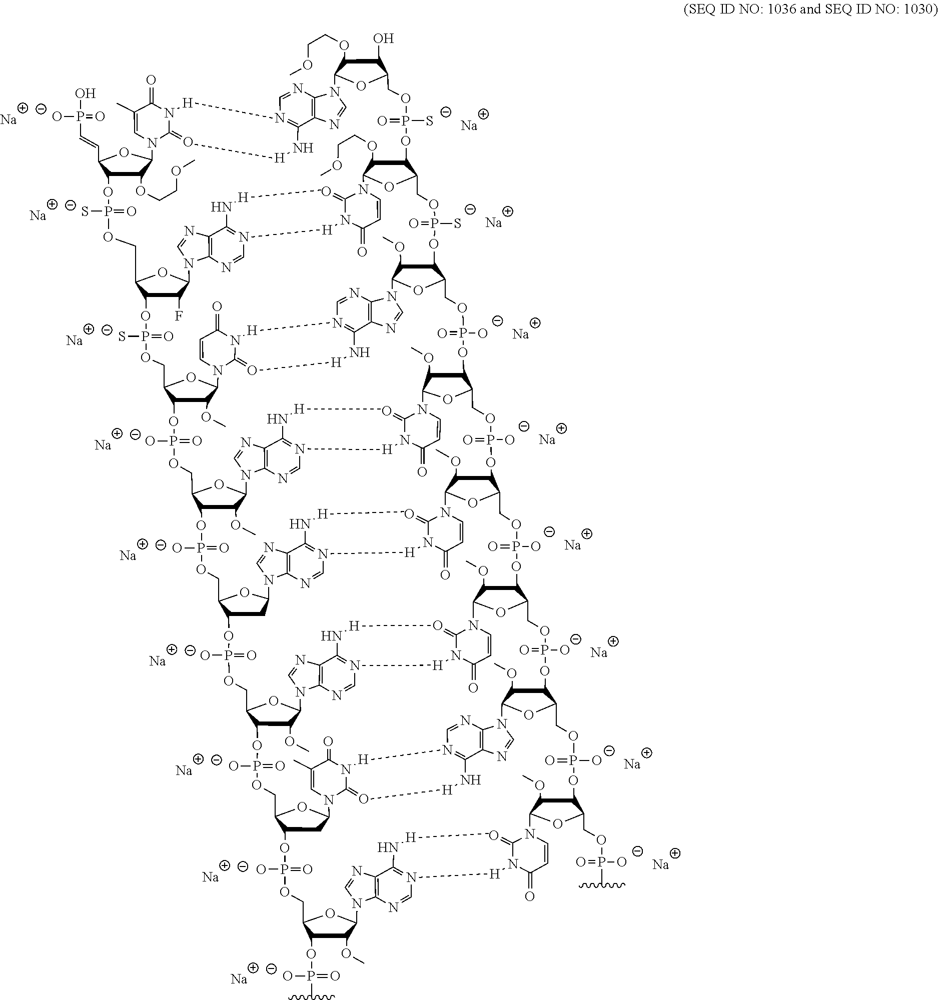

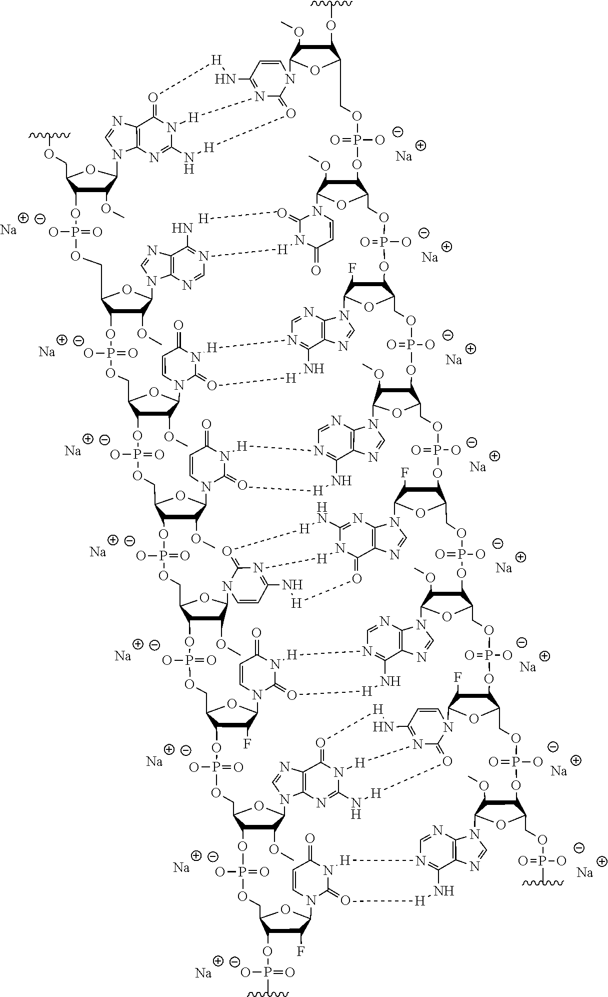

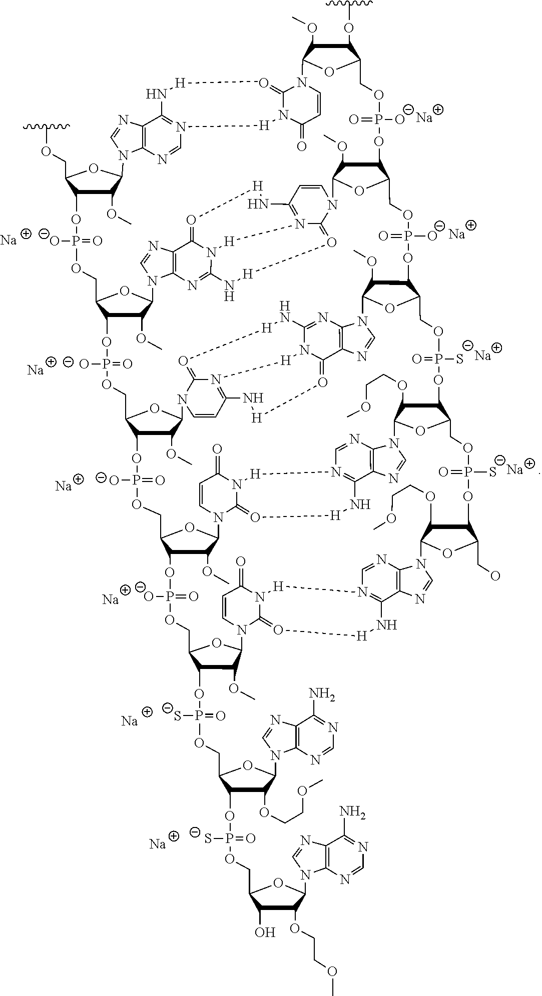

e. Compound No. 1757477

Provided herein is Compound No. 1757477, which is an oligomeric duplex that consists of a first oligomeric compound containing a first modified oligonucleotide and a second oligomeric compound containing a second modified oligonucleotide attached to a conjugate group as follows.

### First Oligomeric Compound of Compound No. 1757477

The first modified oligonucleotide of compound no. 1757477, which is Compound No. 1753977, has a nucleobase sequence of (from 5′ to 3′) TUAAGTUUUAGUCUUAAUCUUAA (SEQ ID NO: 971), wherein each of nucleosides 1, 22 and 23 (from 5′ to 3′) comprises a 2′-MOE modified sugar moiety, each of nucleosides 2, 14 and 16 (from 5′ to 3′) comprises a 2′-fluoro modified sugar moiety, each of nucleosides 3-5, 7-13, 15 and 17-21 (from 5′ to 3′) comprises a 2′-OMe modified sugar moiety, and nucleoside 6 (from 5′ to 3′) is a 2′-deoxynucleoside, wherein each of the internucleoside linkages linking nucleosides 1 to 2, 2 to 3, 21 to 22, and 22 to 23 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, 18 to 19, 19 to 20, and 20 to 21 (from 5′ to 3′) is a phosphodiester linkage, and wherein nucleoside 1 (from 5′ to 3′) is attached to a vinyl phosphonate group.

The first oligomeric compound of Compound 1757477 is represented by the following chemical notation: vPTₑₛU_{fs}A_{yo}A_{yo}G_{yo}T_{do}U_{yo}U_{yo}U_{yo}A_{yo}G_{yo}U_{yo}C_{yo}U_{fo}U_{yo}A_{fo}A_{yo}U_{yo}C_{yo}U_{yo}U_{ys}AₑₛAₑ (SEQ ID NO: 1037); wherein
A=an adenine nucleobase,
C=a cytosine nucleobase,
G=a guanine nucleobase,
T=a thymine nucleobase,
U=a uracil nucleobase,
d=a 2′-deoxy sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate group.

### Second Oligomeric Compound of Compound No. 1757477

The second modified oligonucleotide of Compound No. 1757477, which is Compound No. 1757460, has a nucleobase sequence of (from 5′ to 3′) AAGAUUAAGACUAAAACUUAA (SEQ ID NO: 1015), wherein each of nucleosides 1, 2, 20 and 21 (from 5′ to 3′) comprises a 2′-MOE modified sugar moiety, each of nucleosides 10 and 11 (from 5′ to 3′) comprises a 2′-fluoro modified sugar moiety, and each of nucleosides 3-9, and 12-19 (from 5′ to 3′) comprises a 2′-OMe modified sugar moiety, wherein each of the internucleoside linkages linking nucleosides 1 to 2, 2 to 3, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, and 18 to 19 (from 5′ to 3′) is a phosphodiester linkage, and wherein nucleoside 1 (from 5′ to 3′) is attached through a 5′-phosphodiester linkage and a 6-(BCN-carbamate)hexyl conjugate linker to a conjugate moiety comprising the amino acid sequence of bicycle compound BCY17901 (SEQ ID NO: 1045) wherein each cysteine forms a covalent bond with the molecular scaffold 1,1′,1″-(1,3,5-triazinane-1,3,5-triyl)tris(2-bromoethanone) (TATB).

The second oligomeric compound of Compound 1757477 is represented by the following chemical notation: [X]₁-AₑₛAₑₛG_{yo}A_{yo}U_{yo}U_{yo}A_{yo}A_{yo}G_{yo}A_{fo}C_{fo}U_{yo}A_{yo}A_{yo}A_{yo}A_{yo}C_{yo}U_{yo}U_{ys}AₑₛAₑ (SEQ ID NO: 1043); wherein:
C=a cytosine nucleobase,
A=an adenine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
[X]₁=a conjugate group having the following structure:

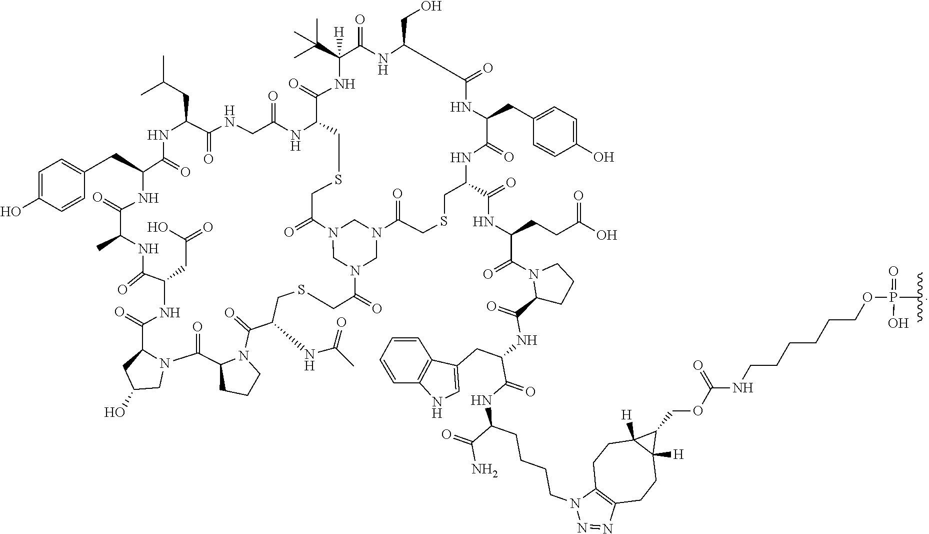 [SMILES: *P(=O)(O)OCCCCCCNC(=O)OC[C@@H]1[C@@H]2CCc3nnn(CCCC[C@H](NC(=O)[C@H](Cc4c[nH]c5ccccc45)NC(=O)[C@@H]4CCCN4C(=O)[C@H](CCC(=O)O)NC(=O)[C@@H]4CSCC(=O)N5CN6CN(C5)C(=O)CSC[C@H](NC(C)=O)C(=O)N5CCC[C@H]5C(=O)N5C[C@H](O)C[C@H]5C(=O)N[C@@H](CC(=O)O)C(=O)N[C@@H](C)C(=O)N[C@@H](Cc5ccc(O)cc5)C(=O)N[C@@H](CC(C)C)C(=O)NCC(=O)N[C@@H](CSCC6=O)C(=O)N[C@@H](C(C)(C)C)C(=O)N[C@@H](CO)C(=O)N[C@@H](Cc5ccc(O)cc5)C(=O)N4)C(N)=O)c3CC[C@@H]21]

The following chemical structure is one structural representation of Compound No. 1757477:

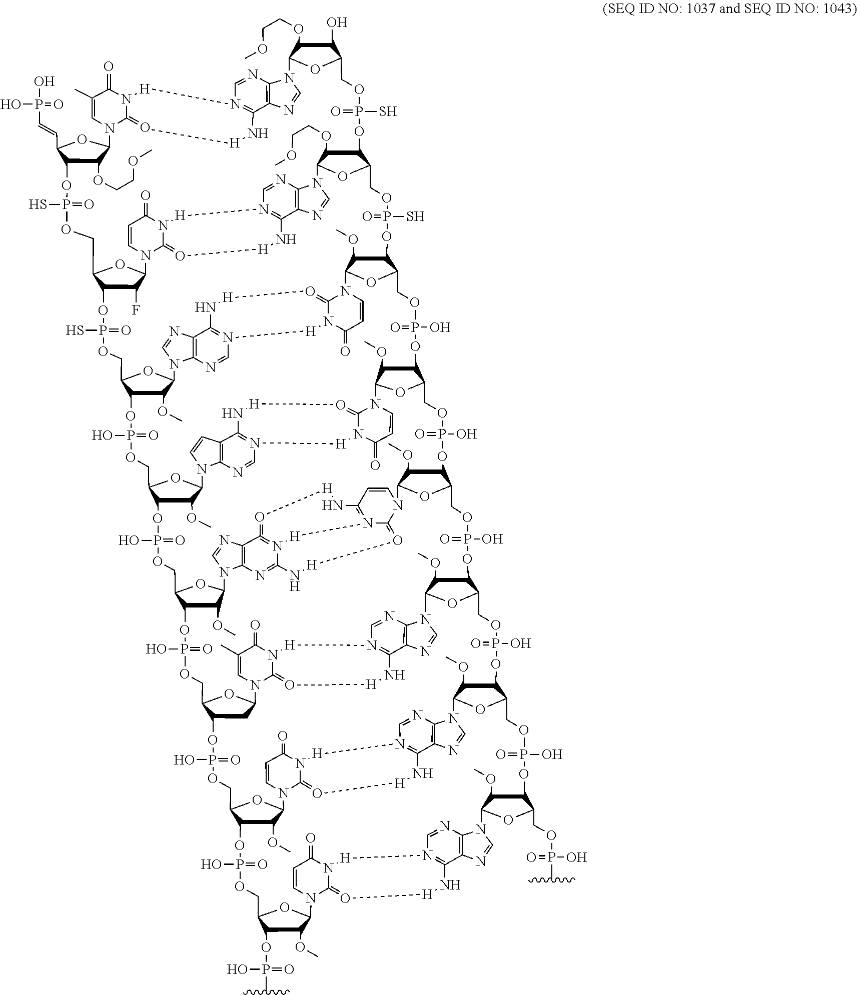

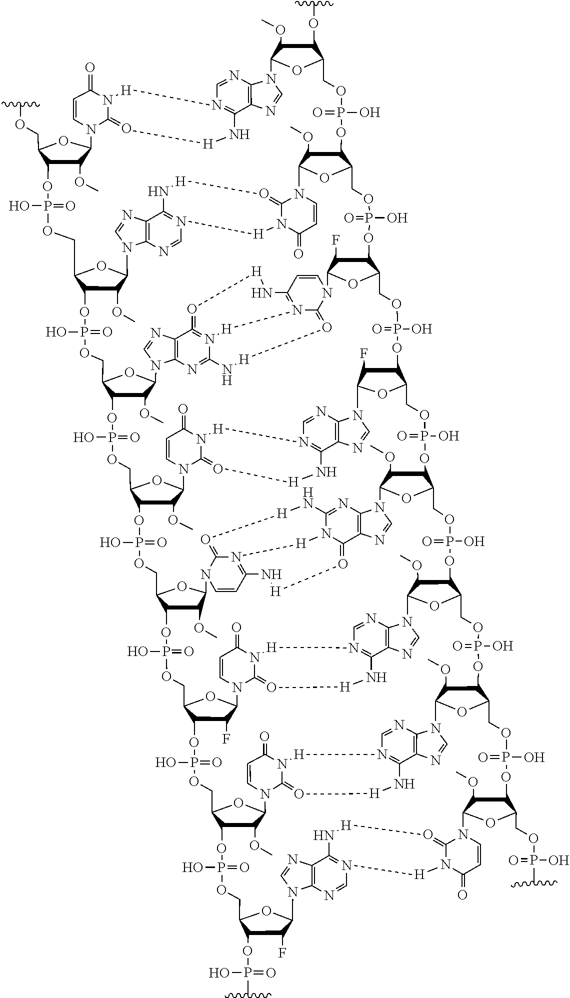

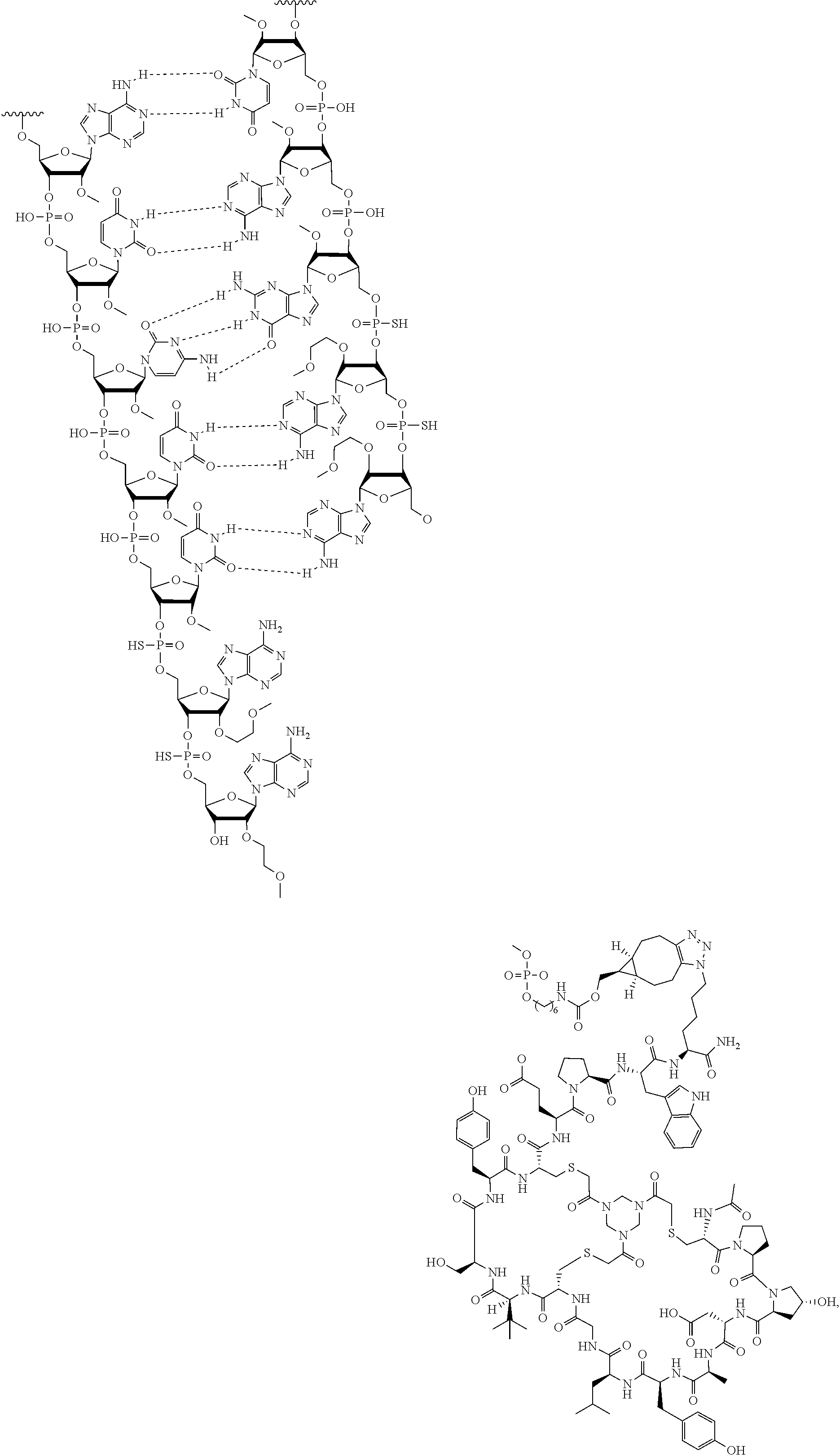

or a salt thereof.

In certain embodiments, Compound No. 1757477 is in the form of an anion or a salt thereof, for example, a sodium salt. In certain embodiments, the oligomeric duplex is in anionic form in a solution. In certain embodiments, Compound No. 1757477 is a sodium salt or a potassium salt.

The following chemical structure represents Compound No. 1757477 in sodium solution:

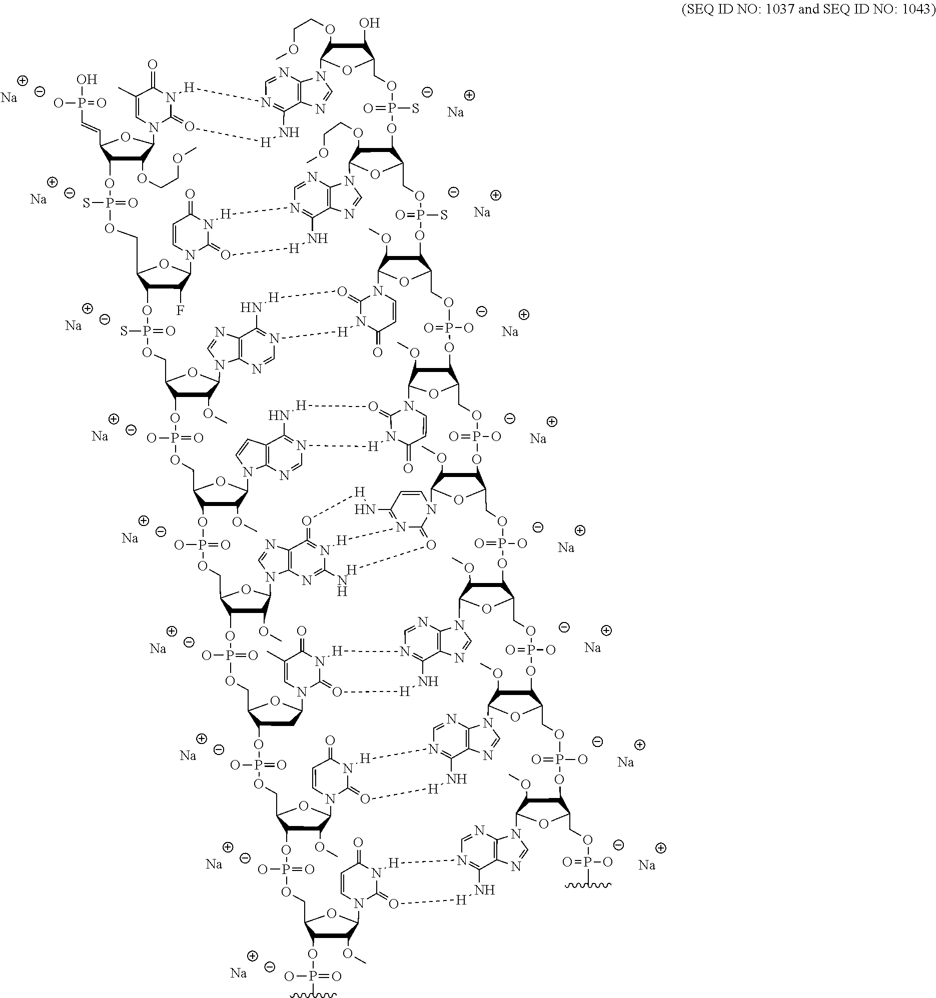

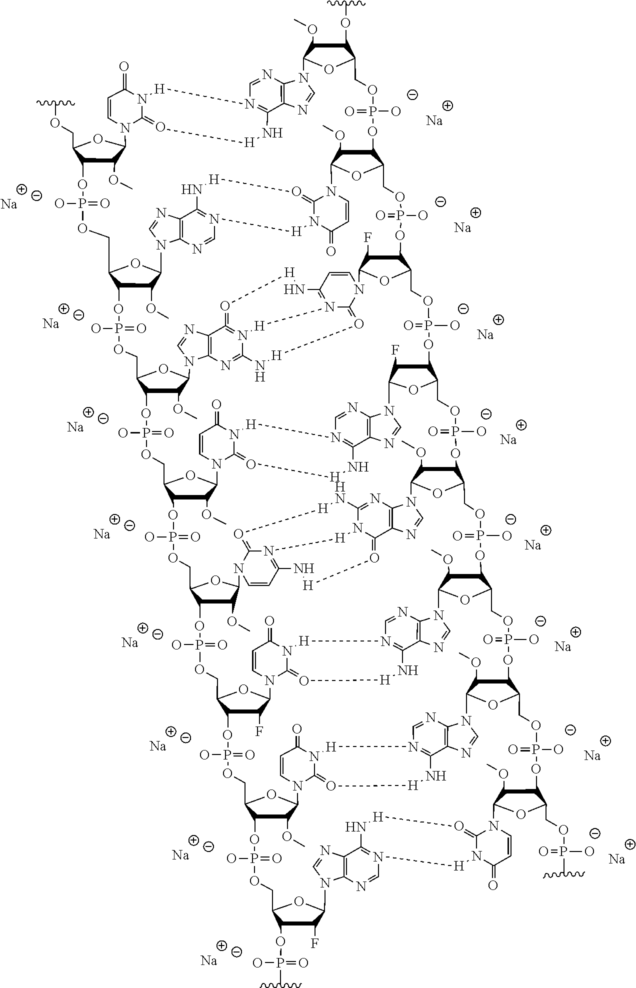

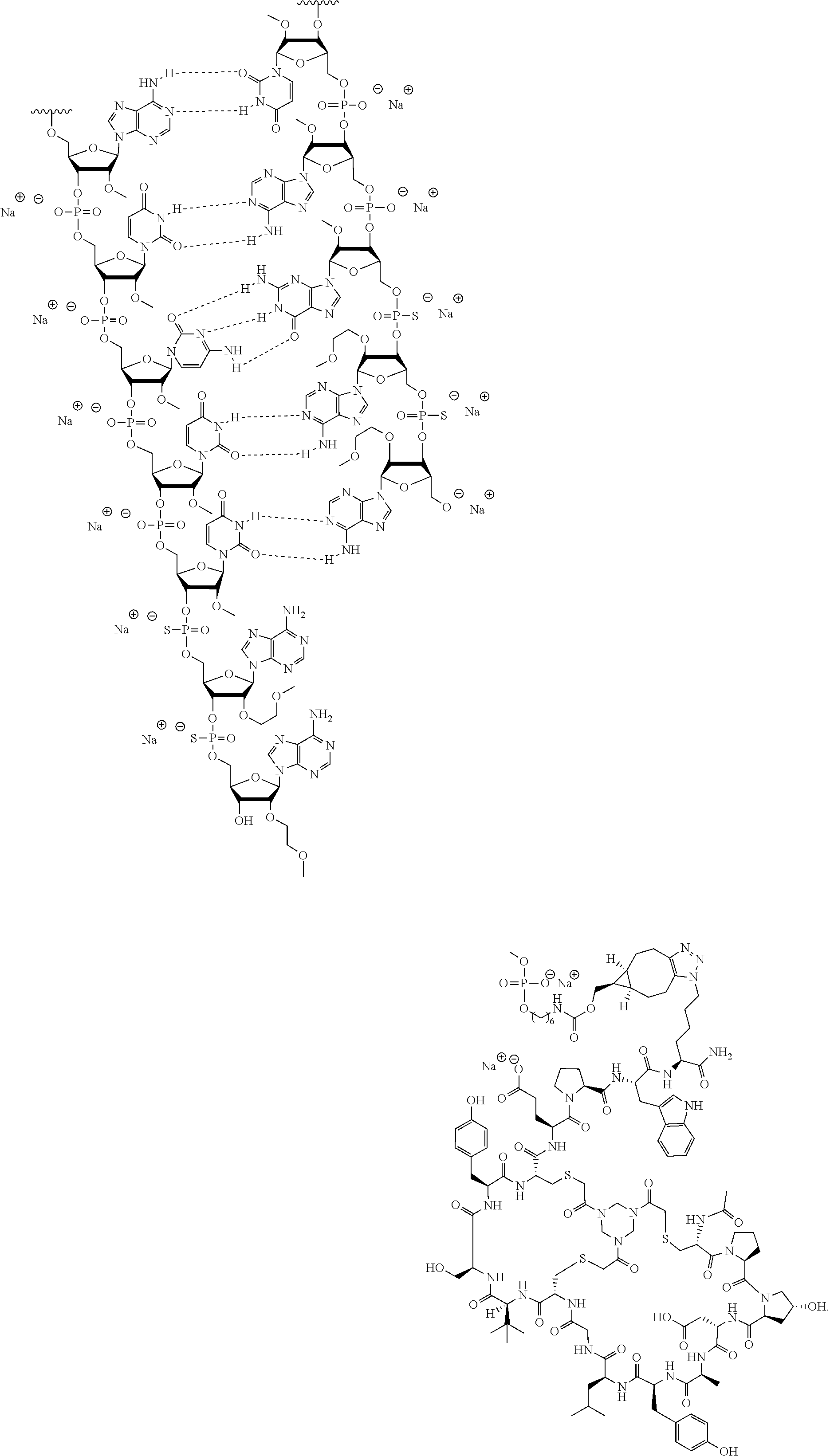

In certain embodiments, provided herein are oligomeric duplexes comprising or consisting of a first oligomeric compound containing a first modified oligonucleotide and a second oligomeric compound containing a second modified oligonucleotide as follows.

### First Oligomeric Compound

The first modified oligonucleotide has a nucleobase sequence of (from 5′ to 3′) TUAAGTUUUAGUCUUAAUCUUAA (SEQ ID NO: 971), wherein each of nucleosides 1, 22 and 23 (from 5′ to 3′) comprises a 2′-MOE modified sugar moiety, each of nucleosides 2, 14 and 16 (from 5′ to 3′) comprises a 2′-fluoro modified sugar moiety, each of nucleosides 3-5, 7-13, 15 and 17-21 (from 5′ to 3′) comprises a 2′-OMe modified sugar moiety, and nucleoside 6 (from 5′ to 3′) is a 2′-deoxynucleoside, wherein each of the internucleoside linkages linking nucleosides 1 to 2, 2 to 3, 21 to 22, and 22 to 23 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, 18 to 19, 19 to 20, and 20 to 21 (from 5′ to 3′) is a phosphodiester linkage, and wherein nucleoside 1 (from 5′ to 3′) is attached to a vinyl phosphonate group.

The first oligomeric compound is represented by the following chemical notation:

(SEQ ID NO: 1037)
vPTₑₛU_{fs}A_{yo}A_{yo}G_{yo}T_{do}U_{yo}U_{yo}U_{yo}A_{yo}G_{yo}U_{yo}C_{yo}U_{fo}U_{yo}A_{fo}
A_{yo}U_{yo}C_{yo}U_{yo}U_{ys}AₑₛAₑ;

A=an adenine nucleobase,
C=a cytosine nucleobase,
G=a guanine nucleobase,
T=a thymine nucleobase,
U=a uracil nucleobase,
d=a 2′-deoxy sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate group.

### Second Oligomeric Compound

The second modified oligonucleotide has a nucleobase sequence of (from 5′ to 3′) AAGAUUAAGACUAAAACUUAA (SEQ ID NO: 1015), wherein each of nucleosides 1, 2, 20 and 21 (from 5′ to 3′) comprises a 2′-MOE modified sugar moiety, each of nucleosides 10 and 11 (from 5′ to 3′) comprises a 2′-fluoro modified sugar moiety, and each of nucleosides 3-9, and 12-19 (from 5′ to 3′) comprises a 2′-OMe modified sugar moiety, wherein each of the internucleoside linkages linking nucleosides 1 to 2, 2 to 3, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, and 18 to 19 (from 5′ to 3′) is a phosphodiester linkage.

The second oligomeric compound is represented by the following chemical notation:

(SEQ ID NO: 1031)
AₑₛAₑₛG_{yo}A_{yo}U_{yo}U_{yo}A_{yo}A_{yo}G_{yo}A_{fo}C_{fo}U_{yo}A_{yo}A_{yo}A_{yo}A_{yo}
C_{yo}U_{yo}U_{ys}AₑₛAₑ;

wherein:
A=an adenine nucleobase,
C=a cytosine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

In particular embodiments, such an oligomeric duplex is represented by the following chemical structure:

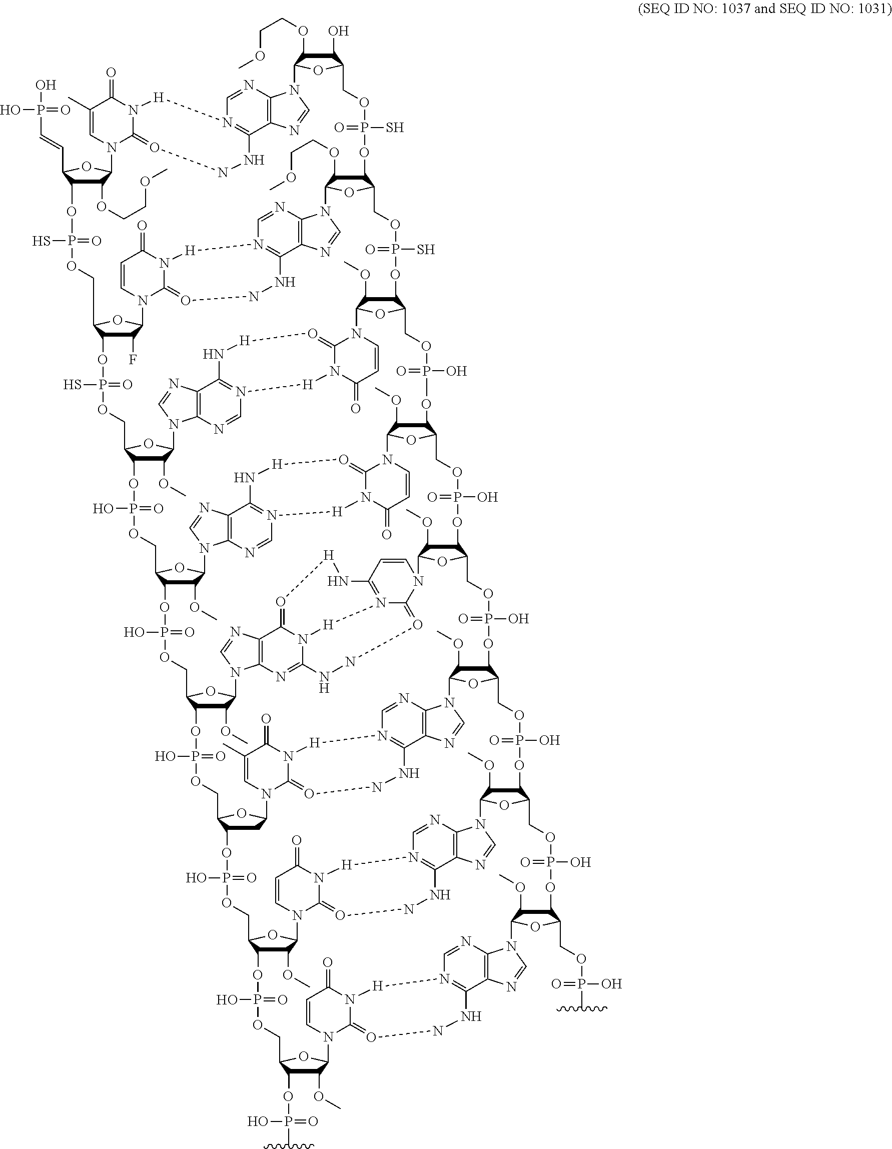

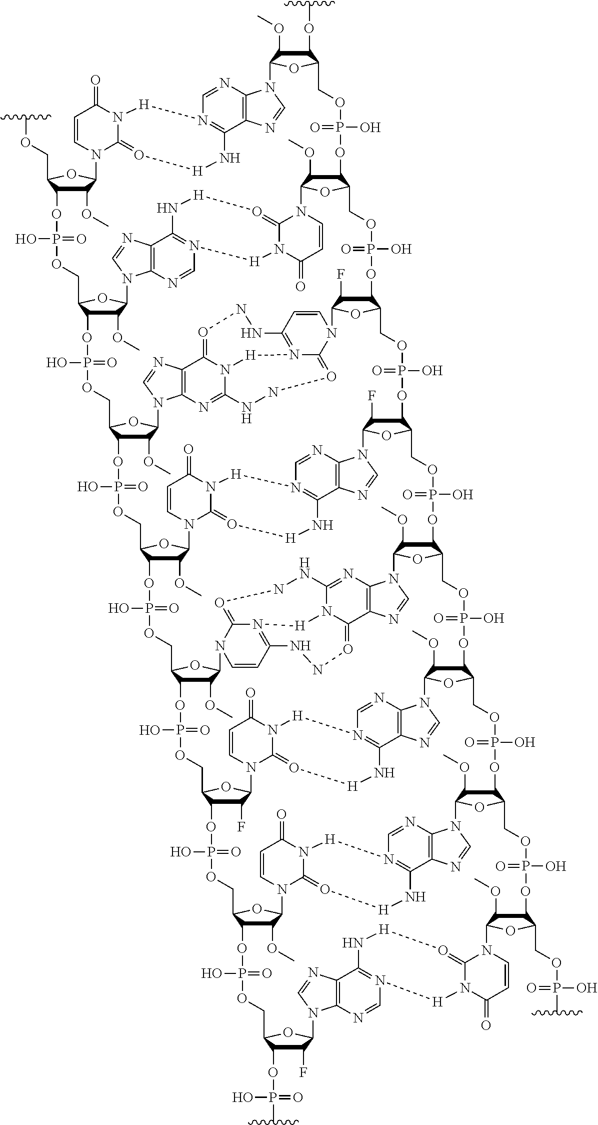

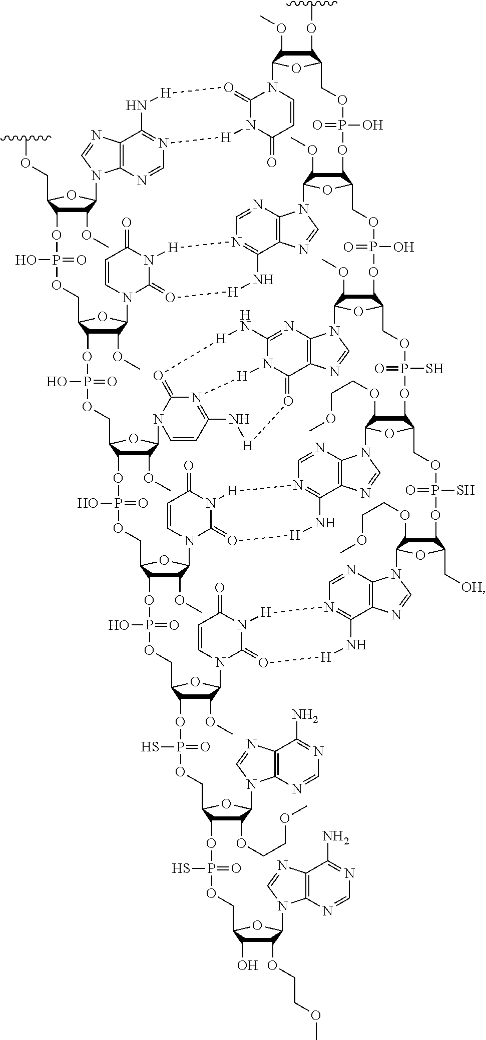

or a salt thereof.

In certain embodiments, such an oligomeric duplex is in the form of an anion or a salt thereof, for example, a sodium salt. In certain embodiments, the oligomeric duplex is in anionic form in a solution. In certain embodiments, the oligomeric duplex is a sodium salt or a potassium salt.

The following chemical structure represents such an oligomeric duplex in sodium solution:

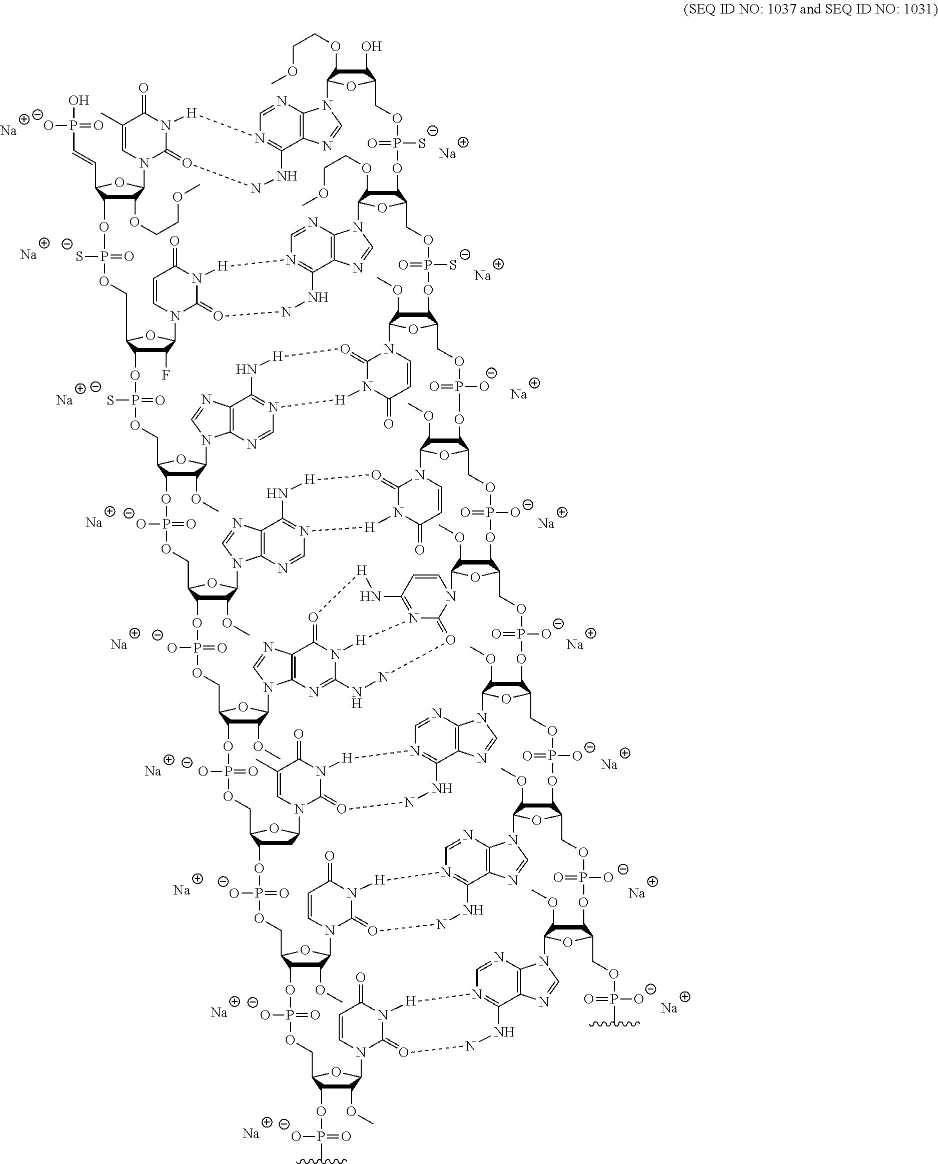

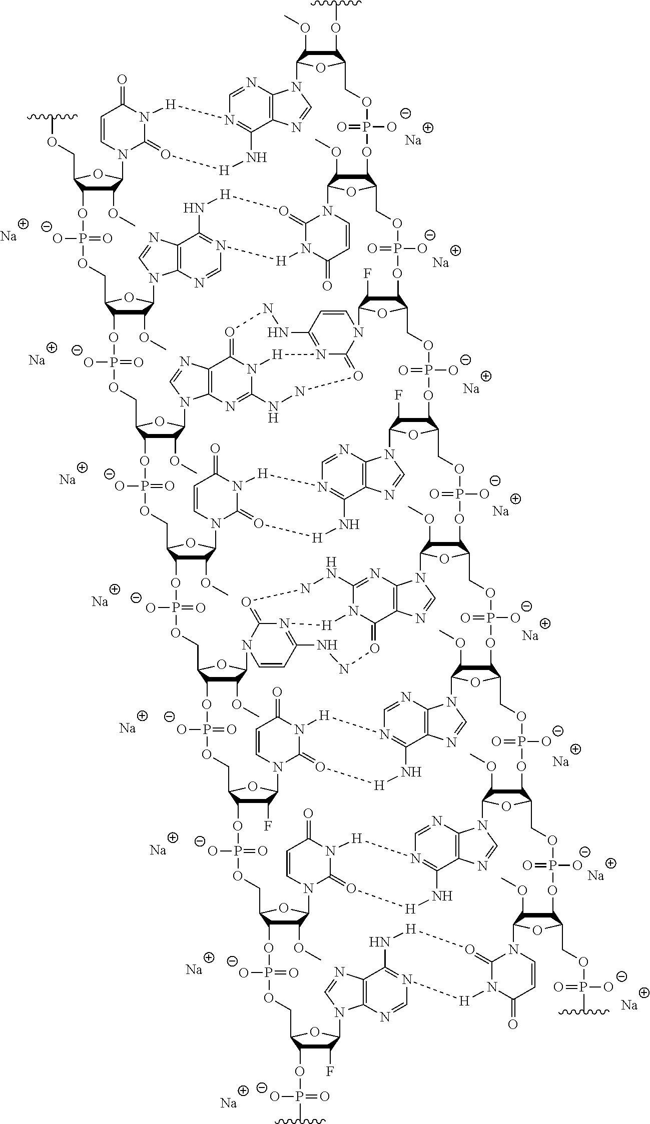

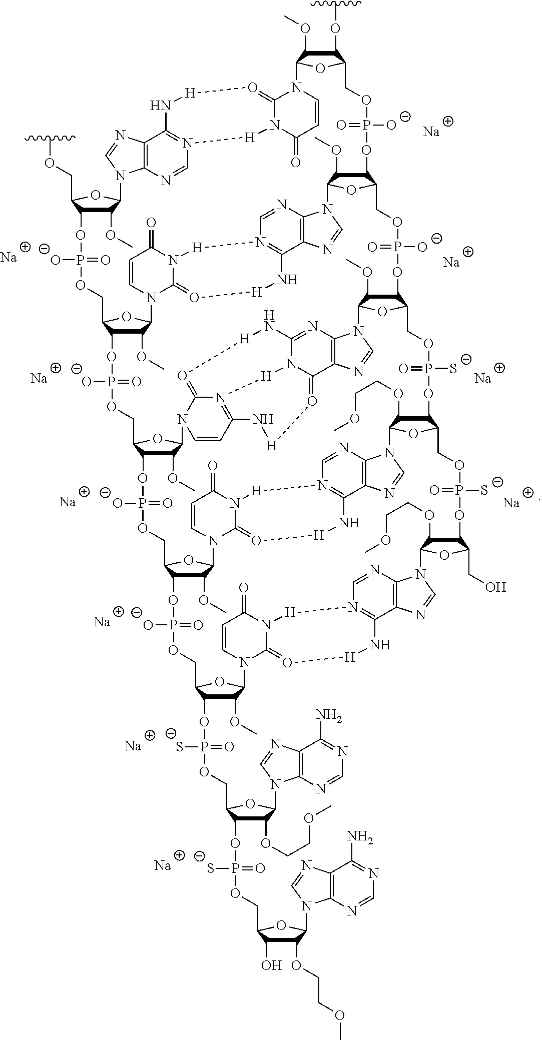

f. Compound No. 1757480

Provided herein is Compound No. 1757480, which is an oligomeric duplex that consists of a first oligomeric compound containing a first modified oligonucleotide and a second oligomeric compound containing a second modified oligonucleotide attached to a conjugate group as follows.

### First Oligomeric Compound of Compound No. 1757480

The first modified oligonucleotide of Compound No. 1757480, which is Compound No. 1753978, has a nucleobase sequence of (from 5′ to 3′) TUAAGUTUUAGUCUUAAUCUUAA (SEQ ID NO: 972), wherein each of nucleosides 1, 22 and 23 (from 5′ to 3′) comprises a 2′-MOE modified sugar moiety, each of nucleosides 2, 14 and 16 (from 5′ to 3′) comprises a 2′-fluoro modified sugar moiety, each of nucleosides 3, 4, 6, 8-13, 15 and 17-21 (from 5′ to 3′) comprises a 2′-OMe modified sugar moiety, and each of nucleosides 5 and 7 (from 5′ to 3′) is a 2′-deoxynucleoside, wherein each of the internucleoside linkages linking nucleosides 1 to 2, 2 to 3, 21 to 22, and 22 to 23 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, 18 to 19, 19 to 20, and 20 to 21 (from 5′ to 3′) is a phosphodiester linkage, and wherein nucleoside 1 (from 5′ to 3′) is attached to a vinyl phosphonate group.

The first oligomeric compound of Compound 1757480 is represented by the following chemical notation: vPTₑₛU_{fs}A_{yo}A_{yo}G_{do}U_{yo}T_{do}U_{yo}U_{yo}A_{yo}G_{yo}U_{yo}C_{yo}U_{fo}U_{yo}A_{fo}A_{yo}U_{yo}C_{yo}U_{yo}U_{ys}AₑₛAₑ (SEQ ID NO: 1038); wherein
A=an adenine nucleobase,
C=a cytosine nucleobase,
G=a guanine nucleobase,
T=a thymine nucleobase,
U=a uracil nucleobase,
d=a 2′-deoxy sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate group.

### Second Oligomeric Compound of Compound No. 1757480

The second modified oligonucleotide of Compound No. 1757480, which is Compound No. 1757462, has a nucleobase sequence of (from 5′ to 3′) AAGAUUAAGACUAAAACUUAA (SEQ ID NO: 1016), wherein each of nucleosides 1, 2, 20 and 21 (from 5′ to 3′) comprises a 2′-MOE modified sugar moiety, each of nucleosides 7, 9, and 11 (from 5′ to 3′) comprises a 2′-fluoro modified sugar moiety, and each of nucleosides 3-6, 8, 10, and 12-19 (from 5′ to 3′) comprises a 2′-OMe modified sugar moiety, wherein each of the internucleoside linkages linking nucleosides 1 to 2, 2 to 3, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, and 18 to 19 (from 5′ to 3′) is a phosphodiester linkage, and wherein nucleoside 1 (from 5′ to 3′) is attached through a 5′-phosphodiester linkage and a 6-(BCN-carbamate)hexyl conjugate linker to a conjugate moiety comprising the amino acid sequence of bicycle compound BCY17901 (SEQ ID NO: 1045) wherein each cysteine forms a covalent bond with the molecular scaffold 1,1′,1″-(1,3,5-triazinane-1,3,5-triyl) tris(2-bromoethanone) (TATB).

The second oligomeric compound of Compound 1757480 is represented by the following chemical notation: [X]₁-AₑₛAₑₛG_{yo}A_{yo}U_{yo}U_{yo}A_{fo}A_{yo}G_{fo}A_{yo}C_{fo}U_{yo}A_{yo}A_{yo}A_{yo}A_{yo}C_{yo}U_{yo}U_{ys}AₑₛAₑ (SEQ ID NO: 1044); wherein:
C=a cytosine nucleobase,
A=an adenine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
[X]₁=a conjugate group having the following structure:

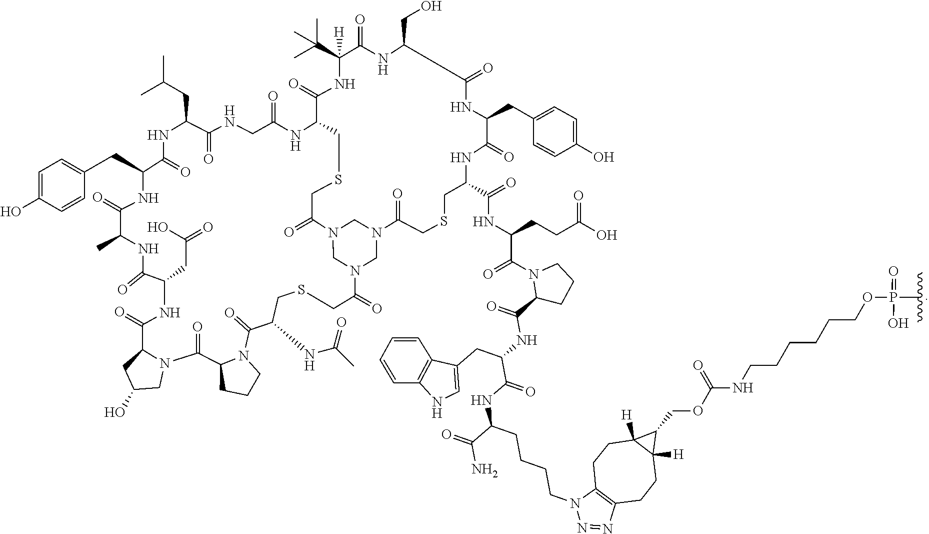 [SMILES: *P(=O)(O)OCCCCCCNC(=O)OC[C@@H]1[C@@H]2CCc3nnn(CCCC[C@H](NC(=O)[C@H](Cc4c[nH]c5ccccc45)NC(=O)[C@@H]4CCCN4C(=O)[C@H](CCC(=O)O)NC(=O)[C@@H]4CSCC(=O)N5CN6CN(C5)C(=O)CSC[C@H](NC(C)=O)C(=O)N5CCC[C@H]5C(=O)N5C[C@H](O)C[C@H]5C(=O)N[C@@H](CC(=O)O)C(=O)N[C@@H](C)C(=O)N[C@@H](Cc5ccc(O)cc5)C(=O)N[C@@H](CC(C)C)C(=O)NCC(=O)N[C@@H](CSCC6=O)C(=O)N[C@@H](C(C)(C)C)C(=O)N[C@@H](CO)C(=O)N[C@@H](Cc5ccc(O)cc5)C(=O)N4)C(N)=O)c3CC[C@@H]21]

The following chemical structure is one structural representation of Compound No. 1757480:

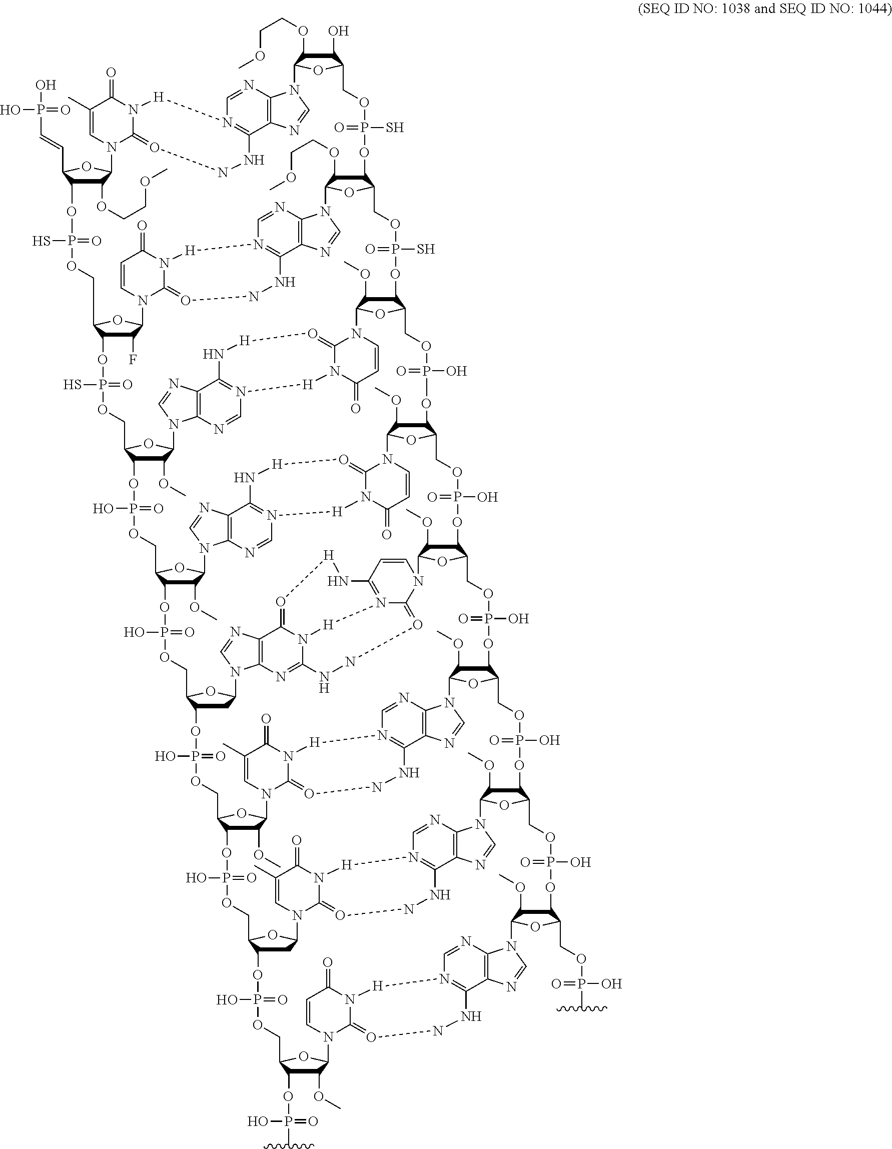

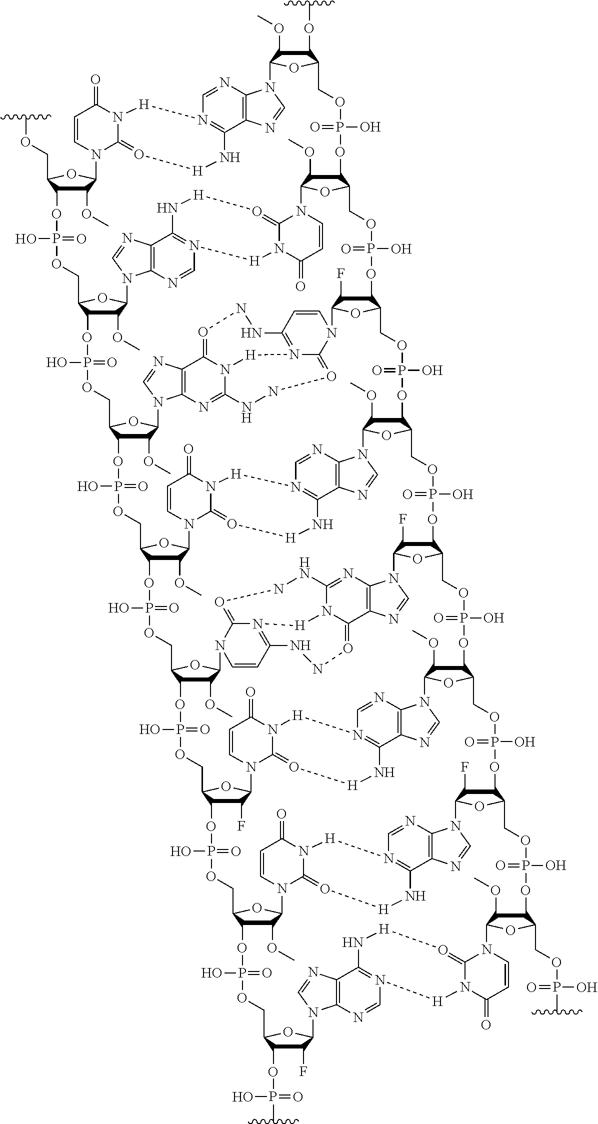

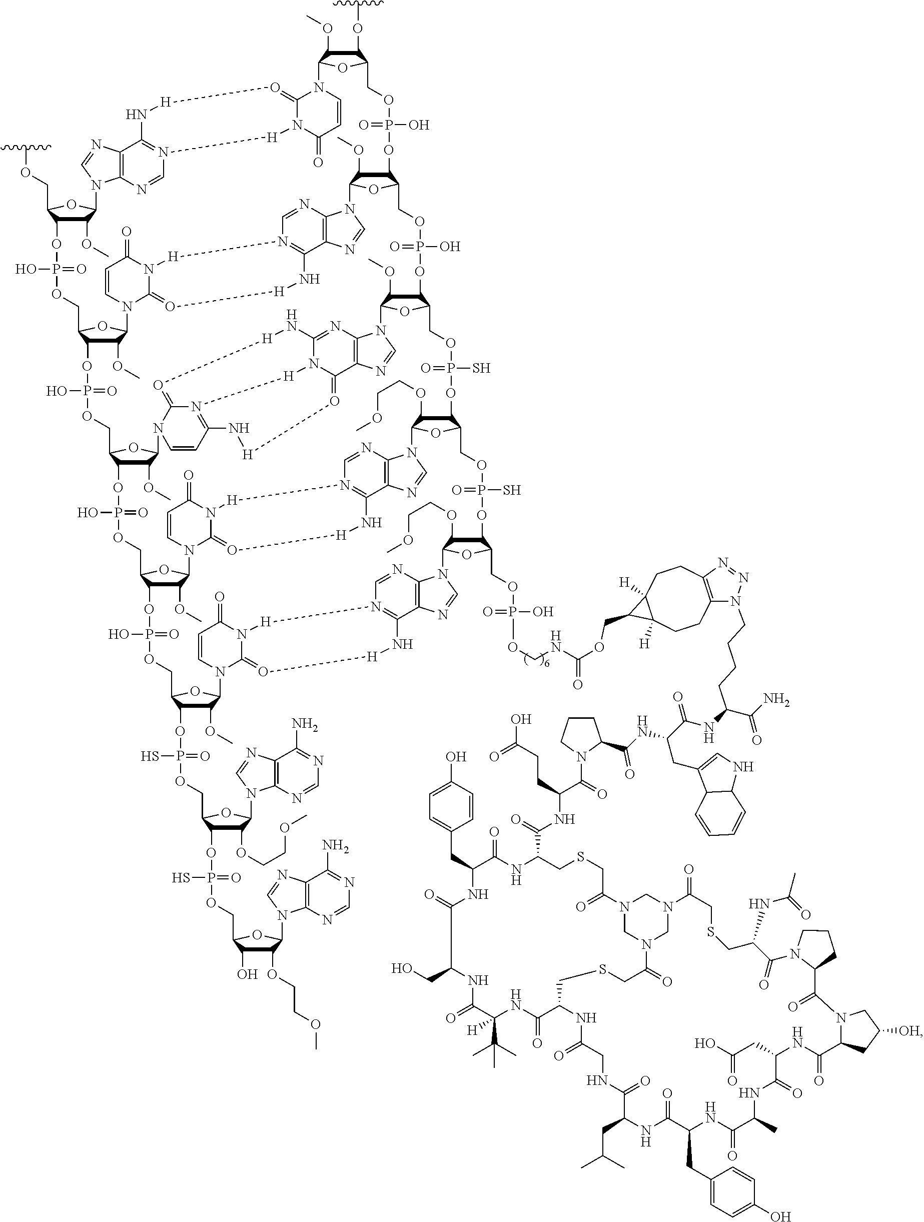

or a salt thereof.

In certain embodiments, Compound No. 1757480 is in the form of an anion or a salt thereof, for example, a sodium salt. In certain embodiments, the oligomeric duplex is in anionic form in a solution. In certain embodiments, Compound No. 1757480 is a sodium salt or a potassium salt.

The following chemical structure represents Compound No. 1757480 in sodium solution can be represented by the following chemical structure:

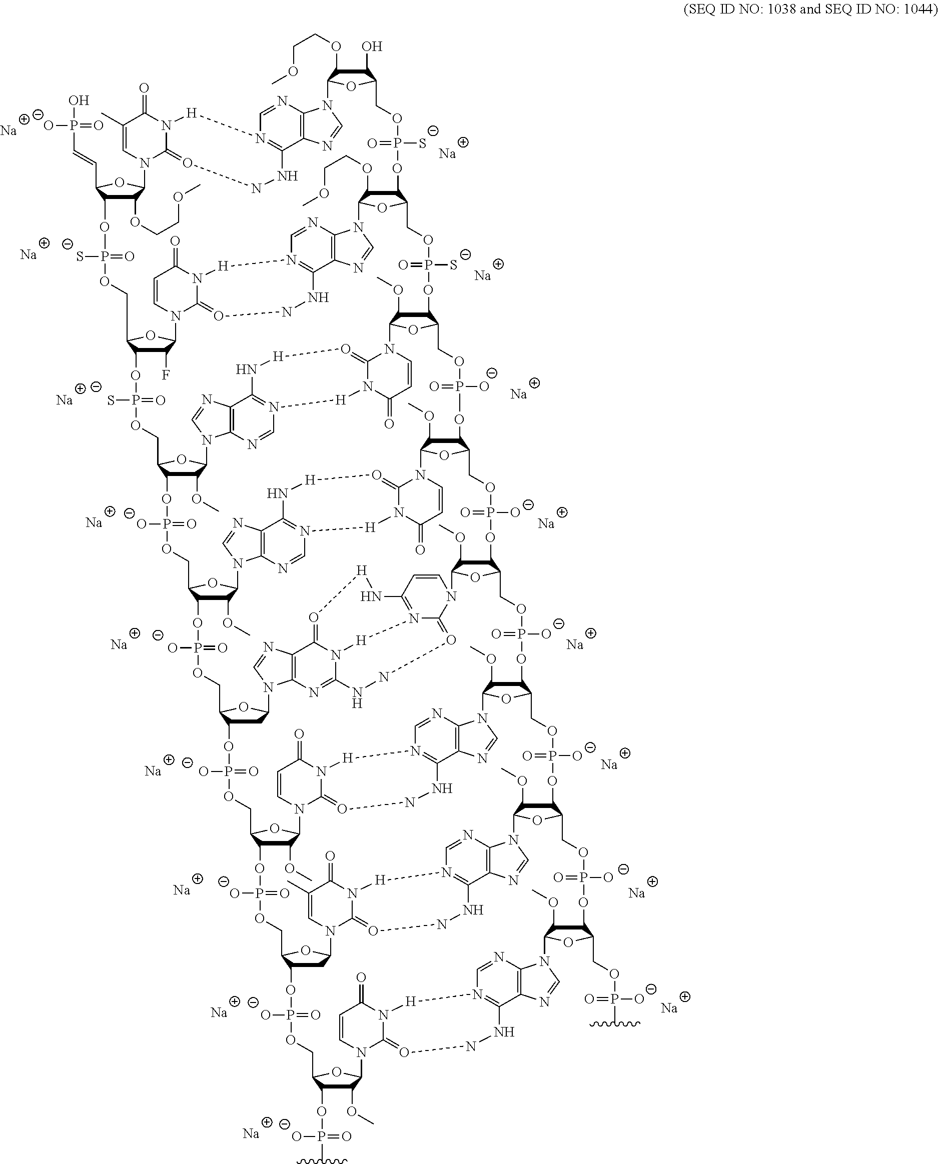

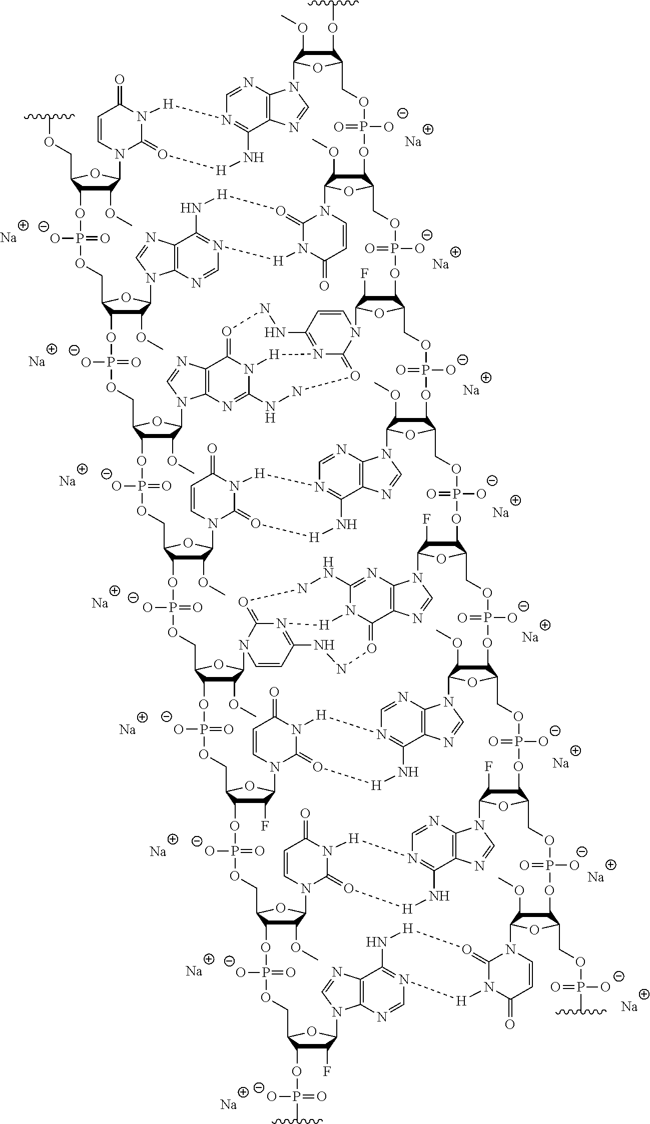

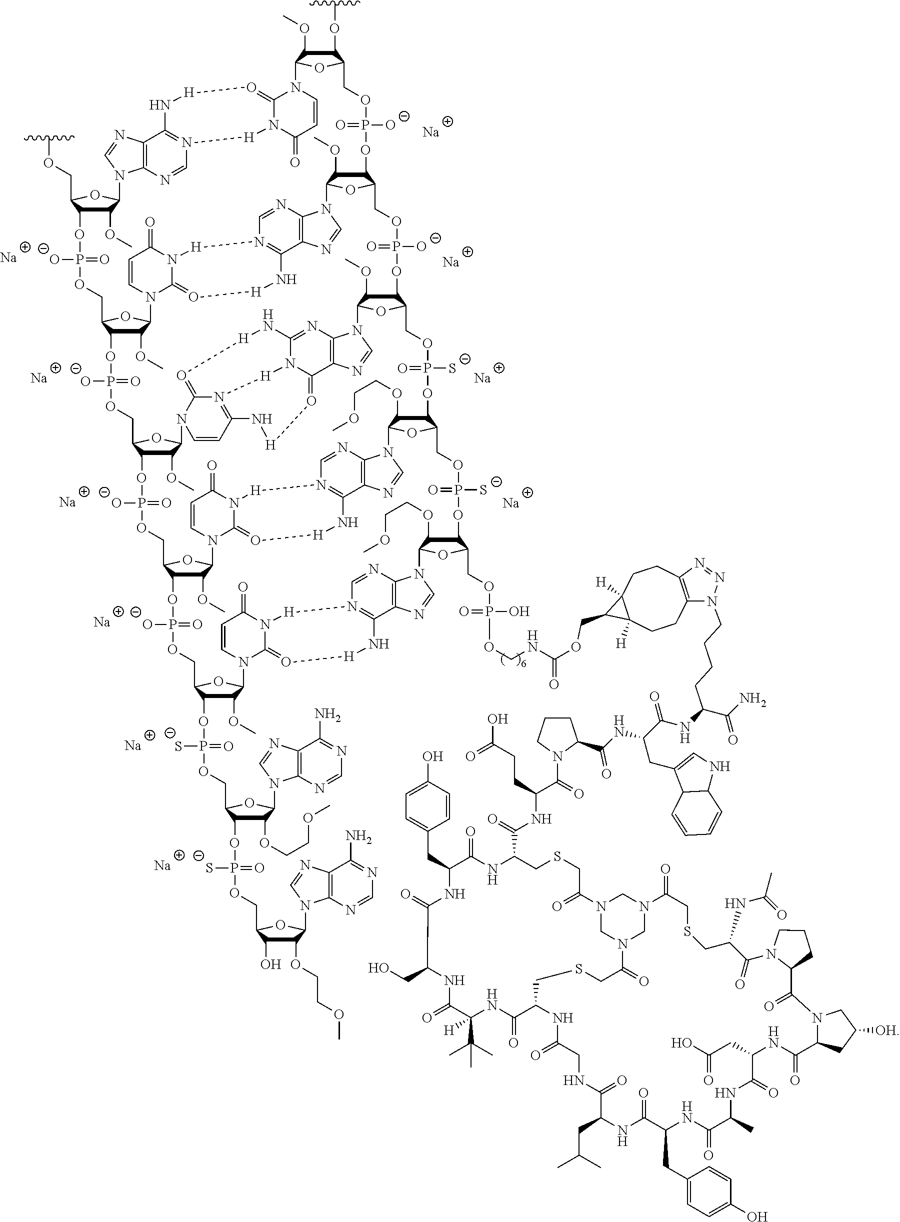

In certain embodiments, provided herein are oligomeric duplexes comprising or consisting of a first oligomeric compound containing a first modified oligonucleotide and a second oligomeric compound containing a second modified oligonucleotide as follows.

### First Oligomeric Compound

The first modified oligonucleotide has a nucleobase sequence of (from 5′ to 3′) TUAAGUTUUAGUCUUAAUCUUAA (SEQ ID NO: 972), wherein each of nucleosides 1, 22 and 23 (from 5′ to 3′) comprises a 2′-MOE modified sugar moiety, each of nucleosides 2, 14 and 16 (from 5′ to 3′) comprises a 2′-fluoro modified sugar moiety, each of nucleosides 3, 4, 6, 8-13, 15 and 17-21 (from 5′ to 3′) comprises a 2′-OMe modified sugar moiety, and each of nucleosides 5 and 7 (from 5′ to 3′) is a 2′-deoxynucleoside, wherein each of the internucleoside linkages linking nucleosides 1 to 2, 2 to 3, 21 to 22, and 22 to 23 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, 18 to 19, 19 to 20, and 20 to 21 (from 5′ to 3′) is a phosphodiester linkage, and wherein nucleoside 1 (from 5′ to 3′) is attached to a vinyl phosphonate group.

The first oligomeric compound is represented by the following chemical notation:

(SEQ ID NO: 1038)
vPTₑₛU_{fs}A_{yo}A_{yo}G_{do}U_{yo}T_{do}U_{yo}U_{yo}A_{yo}G_{yo}U_{yo}C_{yo}U_{fo}U_{yo}A_{fo}
A_{yo}U_{yo}C_{yo}U_{yo}U_{ys}AₑₛAₑ;

A=an adenine nucleobase,
C=a cytosine nucleobase,
G=a guanine nucleobase,
T=a thymine nucleobase,
U=a uracil nucleobase,
d=a 2′-deoxy sugar moiety,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate group.

### Second Oligomeric Compound

The second modified oligonucleotide has a nucleobase sequence of (from 5′ to 3′) AAGAUUAAGACUAAAACUUAA (SEQ ID NO: 1016), wherein each of nucleosides 1, 2, 20 and 21 (from 5′ to 3′) comprises a 2′-MOE modified sugar moiety, each of nucleosides 7, 9, and 11 (from 5′ to 3′) comprises a 2′-fluoro modified sugar moiety, and each of nucleosides 3-6, 8, 10, and 12-19 (from 5′ to 3′) comprises a 2′-OMe modified sugar moiety, wherein each of the internucleoside linkages linking nucleosides 1 to 2, 2 to 3, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, and 18 to 19 (from 5′ to 3′) is a phosphodiester linkage.

The second oligomeric compound is represented by the following chemical notation:

(SEQ ID NO: 1032)
AₑₛAₑₛG_{yo}A_{yo}U_{yo}U_{yo}A_{fo}A_{yo}G_{fo}A_{yo}C_{fo}U_{yo}A_{yo}A_{yo}A_{yo}A_{yo}
C_{yo}U_{yo}U_{ys}AₑₛAₑ,

wherein
A=an adenine nucleobase,
C=a cytosine nucleobase,
G=a guanine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

In particular embodiments, such an oligomeric duplex is represented by the following chemical structure:

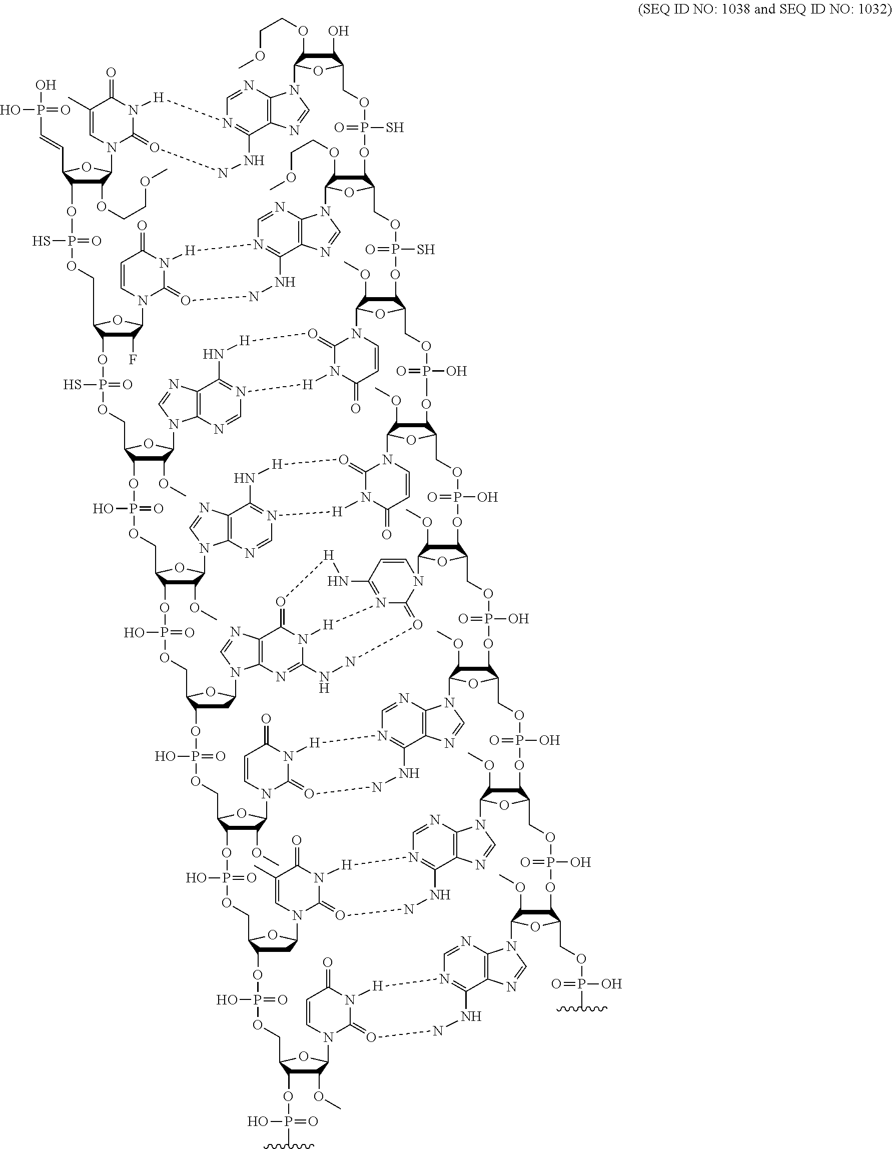

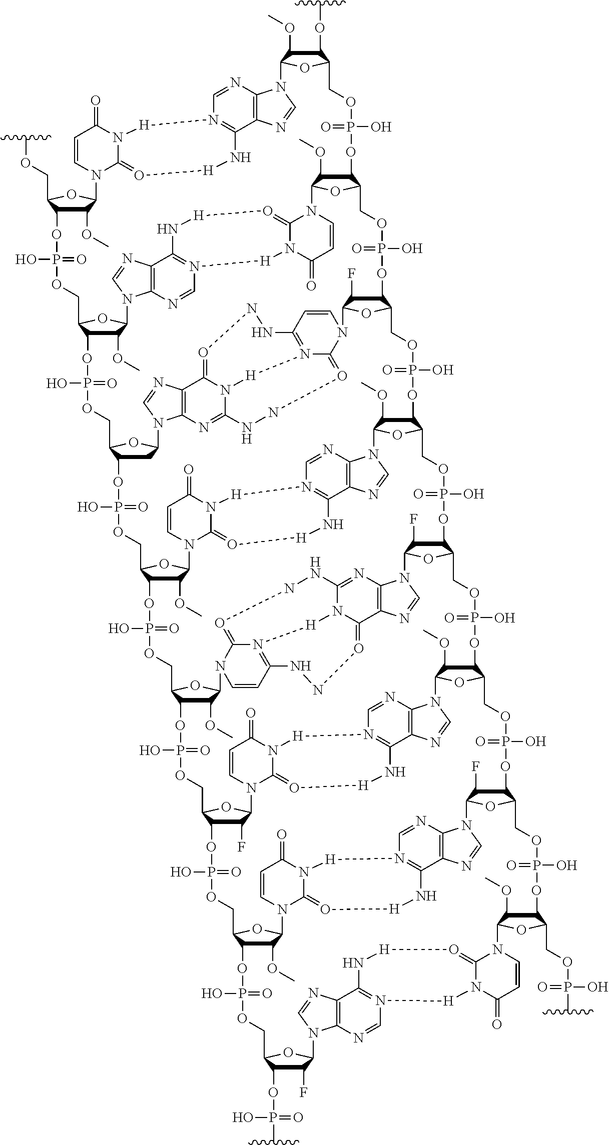

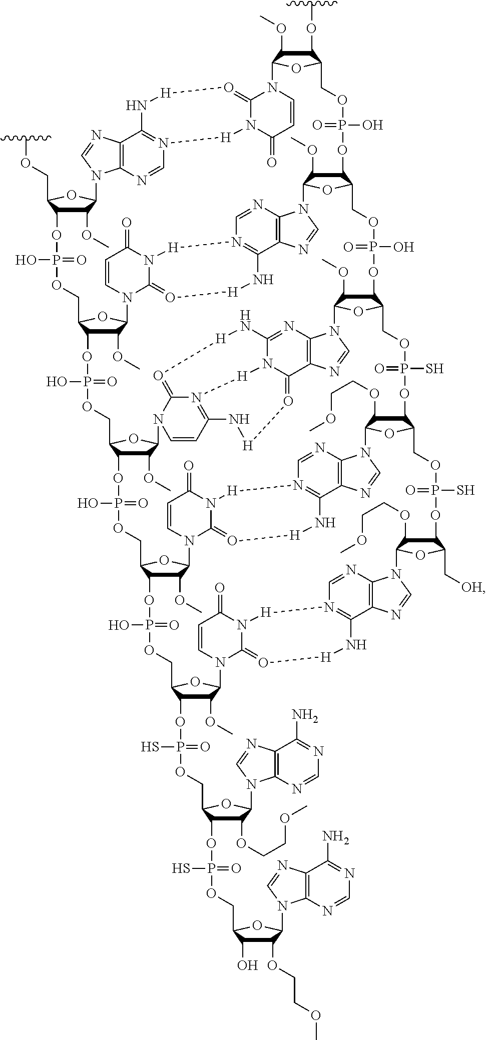

or a salt thereof.

In certain embodiments, such an oligomeric duplex is in the form of an anion or a salt thereof, for example, a sodium salt. In certain embodiments, the oligomeric duplex is in anionic form in a solution. In certain embodiments, the oligomeric duplex is a sodium salt or a potassium salt.

The following chemical structure represents such an oligomeric duplex in sodium solution:

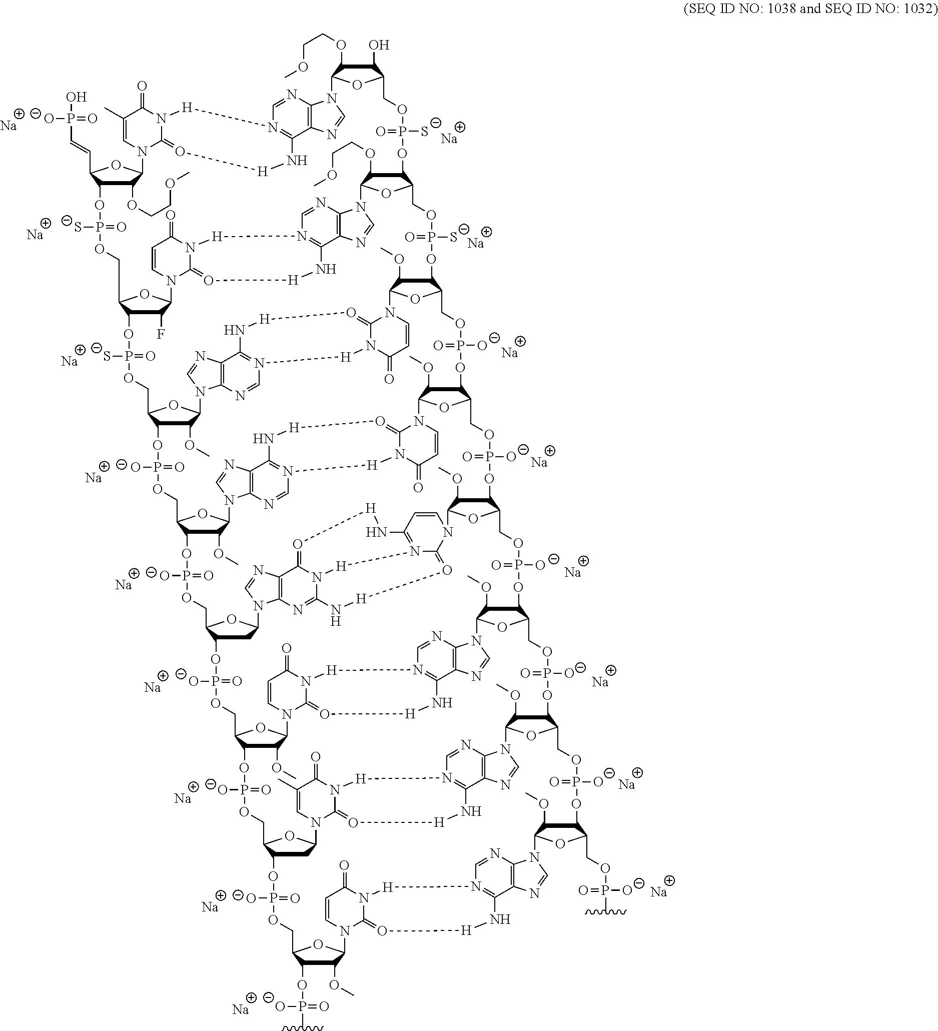

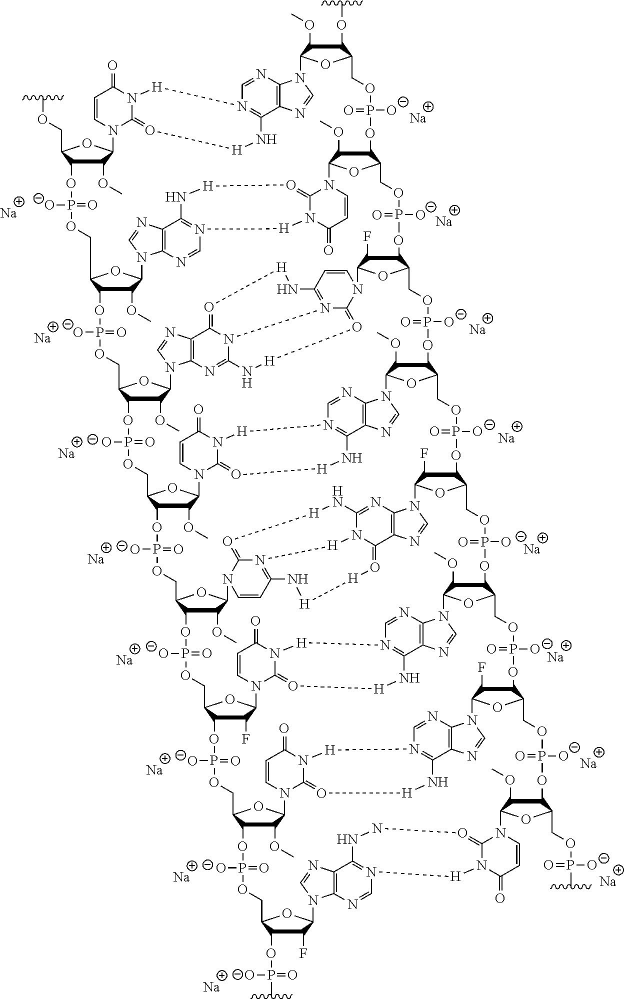

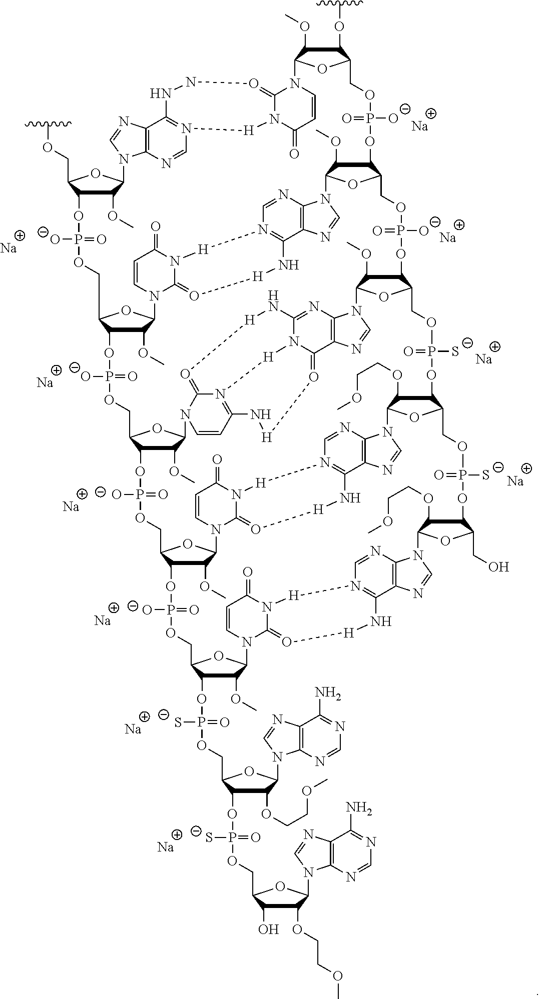

g. Compound No. 1779744

Provided herein is Compound No. 1779744, which is an oligomeric duplex that consists of a first oligomeric compound containing a first modified oligonucleotide and a second oligomeric compound containing a second modified oligonucleotide attached to a conjugate group as follows.

### First Oligomeric Compound of Compound No. 1779744

The first modified oligonucleotide of Compound no. 1779744, which is Compound No. 1753981, has a nucleobase sequence of (from 5′ to 3′) TAUAAAUAGAUUCUGUAGCUUAA (SEQ ID NO: 1254), wherein each of nucleosides 1, 22 and 23 (from 5′ to 3′) comprises a 2′-MOE modified sugar moiety, each of nucleosides 2, 14 and 16 (from 5′ to 3′) comprises a 2′-fluoro modified sugar moiety, and each of nucleosides 3-13, 15 and 17-21 (from 5′ to 3′) comprises a 2′-OMe modified sugar moiety, wherein each of the internucleoside linkages linking nucleosides 1 to 2, 2 to 3, 21 to 22, and 22 to 23 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 1, 11 to 12, 12 to 13, 13 to 14, 14 to 15, 15 to 16, 16 to 17, 17 to 18, 18 to 19, 19 to 20, and 20 to 21 (from 5′ to 3′) is a phosphodiester linkage, and wherein nucleoside 1 (from 5′ to 3′) is attached to a vinyl phosphonate group.

The first oligomeric compound of Compound 1779744 is represented by the following chemical notation: vPTₑₛA_{fs}U_{yo}A_{yo}A_{yo}A_{yo}U_{yo}A_{yo}G_{yo}A_{yo}U_{yo}U_{yo}C_{yo}U_{fo}G_{yo}U_{fo}A_{yo}G_{yo}C_{yo}U_{yo}U_{ys}AₑₛAₑ (SEQ ID NO: 1255); wherein
A=an adenine nucleobase,
C=a cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate group.

### Second Oligomeric Compound of Compound No. 1779744

The second modified oligonucleotide of Compound No. 1779744, which is Compound No. 1757456, has a nucleobase sequence of (from 5′ to 3′) AAGCUACAGAAUCUAUUUATA (SEQ ID NO: 1013), wherein each of nucleosides 1, 2, 20 and 21 (from 5′ to 3′) comprises a 2′-MOE modified sugar moiety, each of nucleosides 10 and 11 (from 5′ to 3′) comprises a 2′-fluoro modified sugar moiety, and each of nucleosides 3-9 and 12-19 (from 5′ to 3′) comprises a 2′-OMe modified sugar moiety, wherein each of the internucleoside linkages linking nucleosides 1 to 2, 2 to 3, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 1, 11 to 12, 12 to 13, 13 to 14, 14 to 15, 15 to 16, 16 to 17, 17 to 18, and 18 to 19 (from 5′ to 3′) is a phosphodiester linkage, and wherein nucleoside 1 (from 5′ to 3′) is attached through a 5′-phosphodiester linkage and a 6-(BCN-carbamate)hexyl conjugate linker to a conjugate moiety comprising the amino acid sequence of bicycle compound BCY17901 (SEQ ID NO: 1045) wherein each cysteine forms a covalent bond with the molecular scaffold 1,1′,1″-(1,3,5-triazinane-1,3,5-triyl)tris(2-bromoethanone) (TATB).

The second oligomeric compound of Compound 1779744 is represented by the following chemical notation: [X]₁-AₑₛAₑₛG_{yo}C_{yo}U_{yo}A_{yo}C_{yo}A_{yo}G_{yo}A_{fo}A_{fo}U_{yo}C_{yo}U_{yo}A_{yo}U_{yo}U_{yo}U_{yo}A_{ys}TₑₛAₑ (SEQ ID NO: 1041); wherein:
C=a cytosine nucleobase,
A=an adenine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
[X]₁=a conjugate group having the following structure:

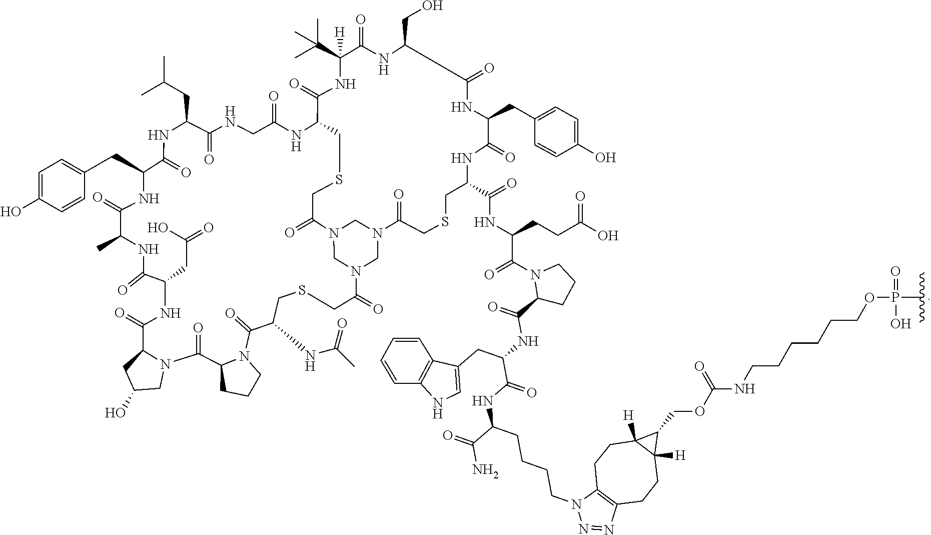 [SMILES: *P(=O)(O)OCCCCCCNC(=O)OC[C@@H]1[C@@H]2CCc3nnn(CCCC[C@H](NC(=O)[C@H](Cc4c[nH]c5ccccc45)NC(=O)[C@@H]4CCCN4C(=O)[C@H](CCC(=O)O)NC(=O)[C@@H]4CSCC(=O)N5CN6CN(C5)C(=O)CSC[C@H](NC(C)=O)C(=O)N5CCC[C@H]5C(=O)N5C[C@H](O)C[C@H]5C(=O)N[C@@H](CC(=O)O)C(=O)N[C@@H](C)C(=O)N[C@@H](Cc5ccc(O)cc5)C(=O)N[C@@H](CC(C)C)C(=O)NCC(=O)N[C@@H](CSCC6=O)C(=O)N[C@@H](C(C)(C)C)C(=O)N[C@@H](CO)C(=O)N[C@@H](Cc5ccc(O)cc5)C(=O)N4)C(N)=O)c3CC[C@@H]21]

The following chemical structure is one structural representation of Compound No. 1779744:

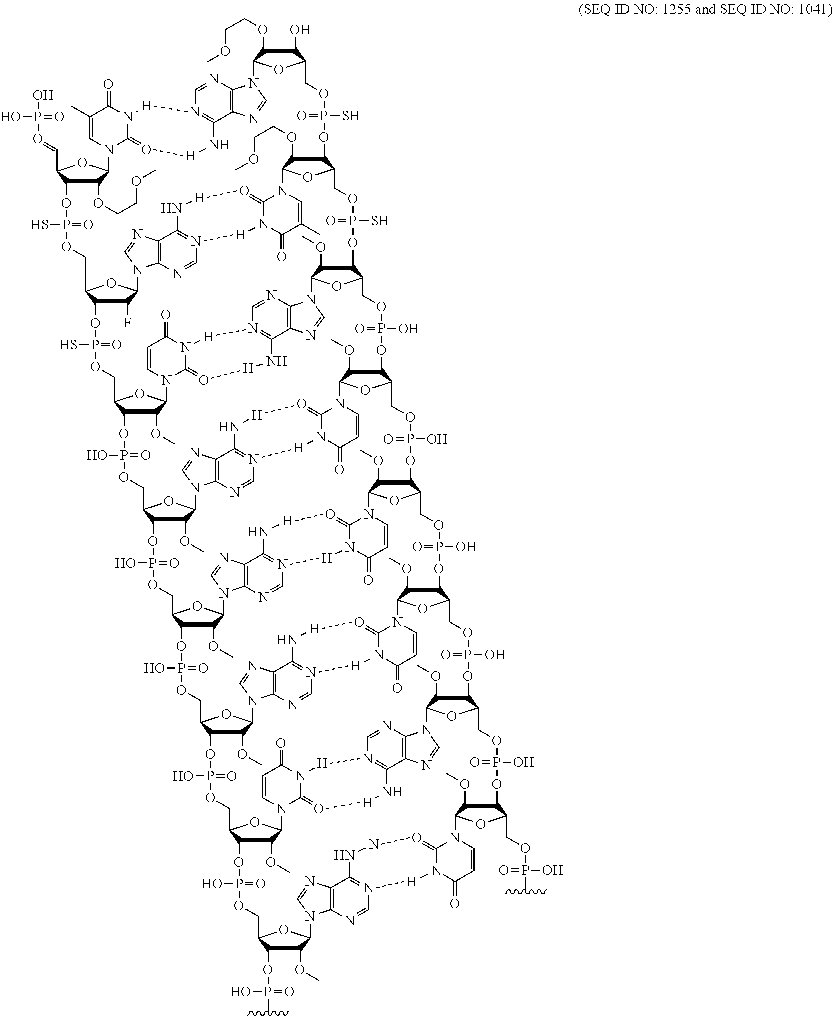

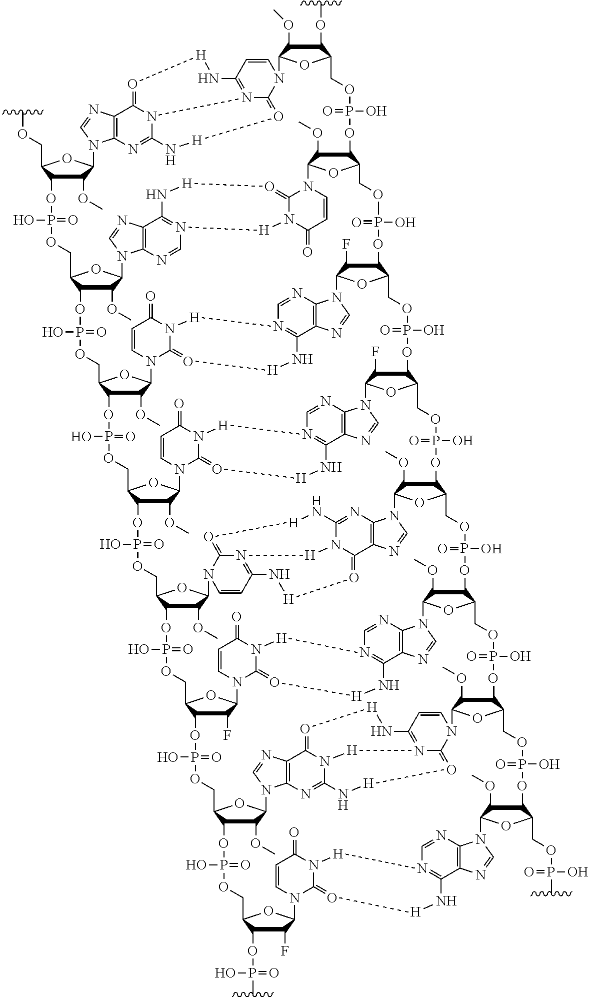

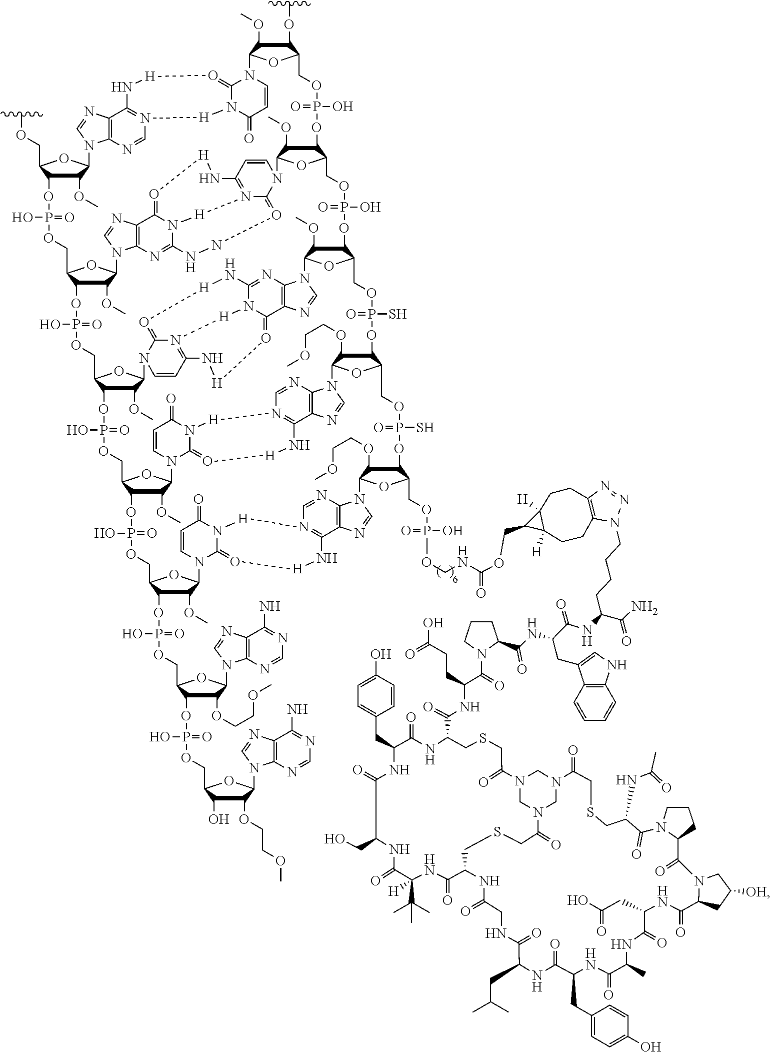

or a salt thereof.

In certain embodiments, Compound No. 1779744 is in the form of an anion or a salt thereof, for example, a sodium salt. In certain embodiments, the oligomeric duplex is in anionic form in a solution. In certain embodiments, Compound No. 1779744 is a sodium salt or a potassium salt. The following chemical structure represents Compound No. 1779744 in sodium solution:

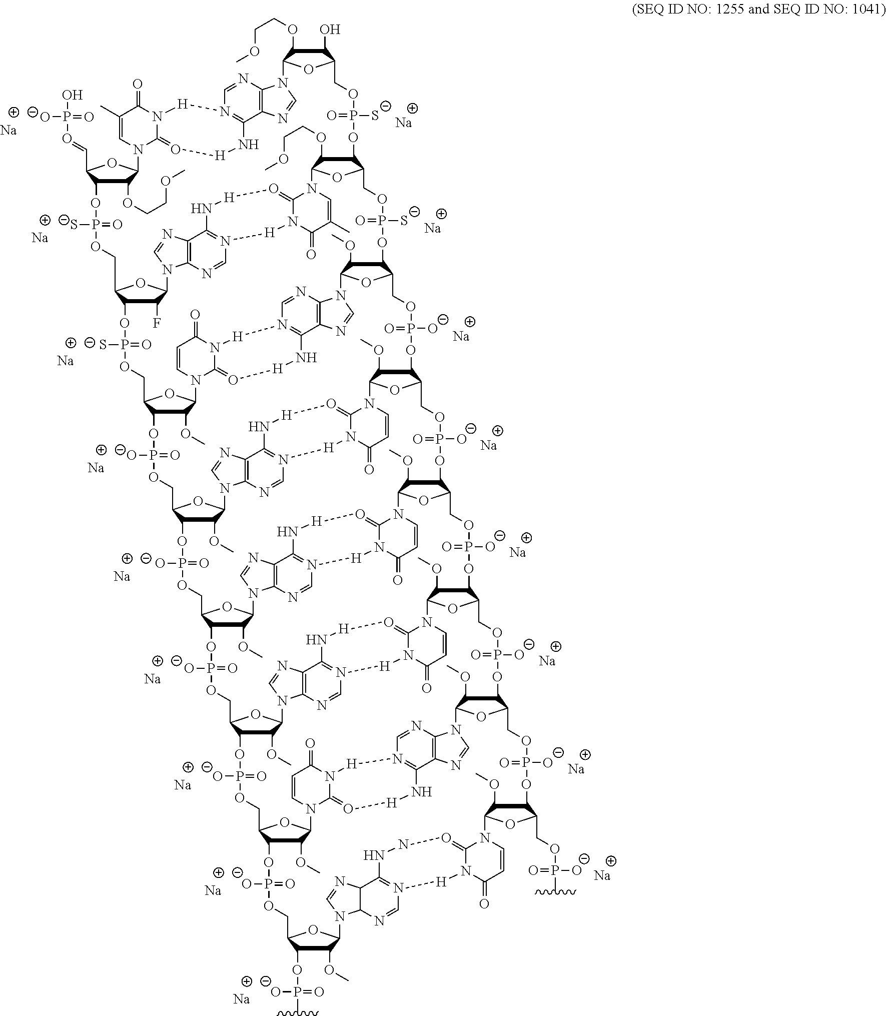

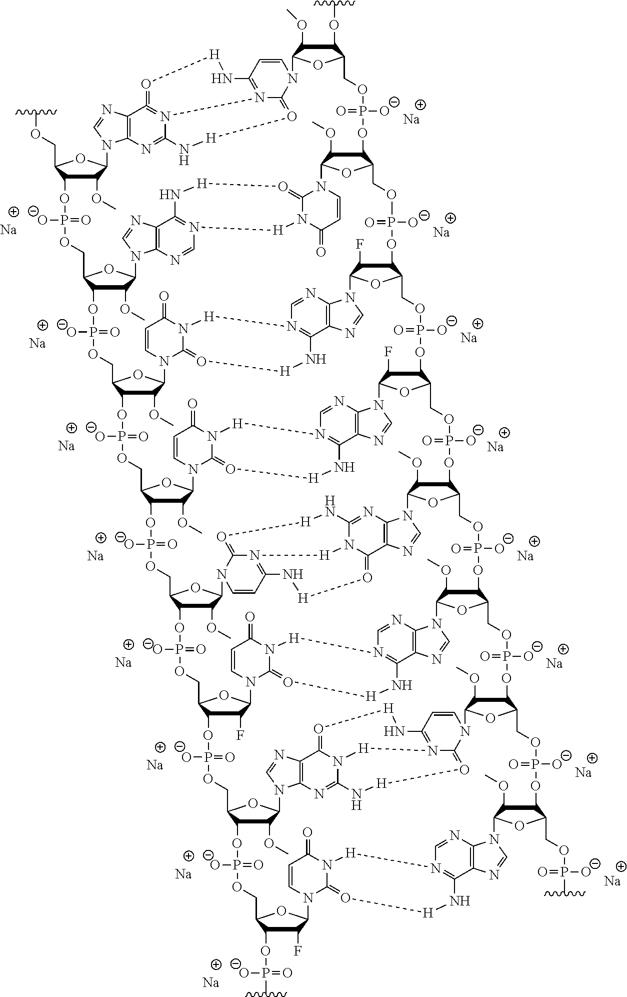

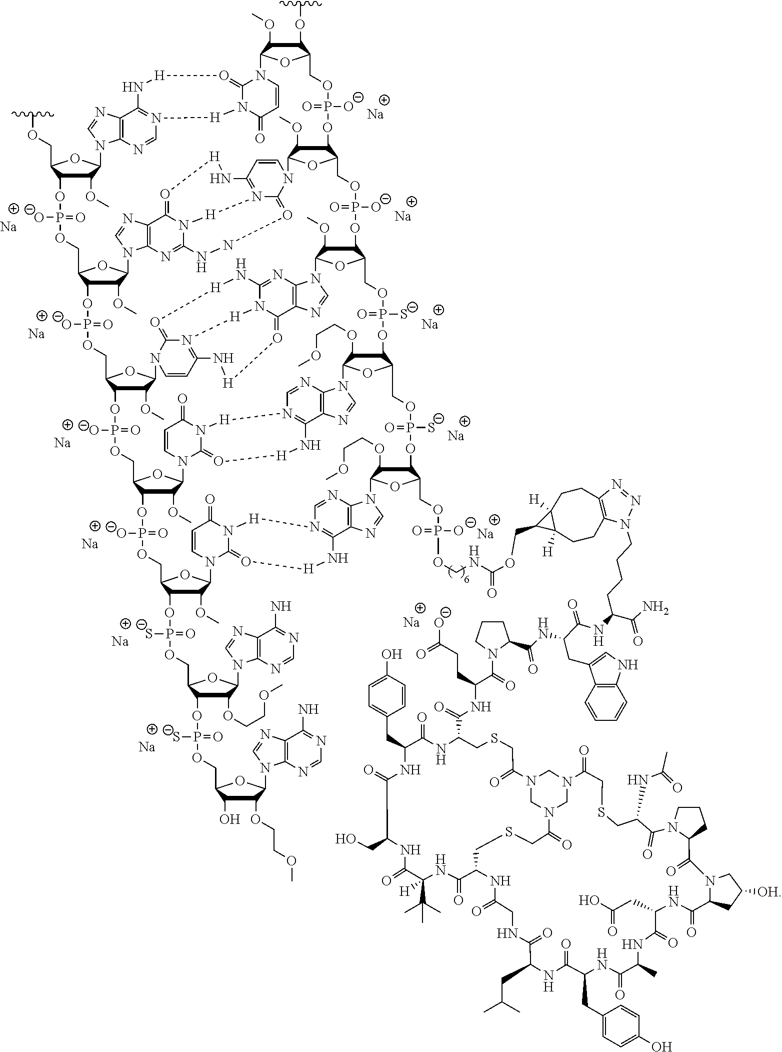

In certain embodiments, provided herein are oligomeric duplexes comprising or consisting of a first oligomeric compound containing a first modified oligonucleotide and a second oligomeric compound containing a second modified oligonucleotide as follows.

### First Oligomeric Compound

The first modified oligonucleotide has a nucleobase sequence of (from 5′ to 3′) TAUAAAUAGAUUCUGUAGCUUAA (SEQ ID NO: 1254), wherein each of nucleosides 1, 22 and 23 (from 5′ to 3′) comprises a 2′-MOE modified sugar moiety, each of nucleosides 2, 14 and 16 (from 5′ to 3′) comprises a 2′-fluoro modified sugar moiety, and each of nucleosides 3-13, 15 and 17-21 (from 5′ to 3′) comprises a 2′-OMe modified sugar moiety, wherein each of the internucleoside linkages linking nucleosides 1 to 2, 2 to 3, 21 to 22, and 22 to 23 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, 18 to 19, 19 to 20, and 20 to 21 (from 5′ to 3′) is a phosphodiester linkage, and wherein nucleoside 1 (from 5′ to 3′) is attached to a vinyl phosphonate group.

The first oligomeric compound is represented by the following chemical notation:

(SEQ ID NO: 1255)
vPTₑₛA_{fs}U_{yo}A_{yo}A_{yo}A_{yo}U_{yo}A_{yo}G_{yo}A_{yo}U_{yo}U_{yo}C_{yo}U_{fo}G_{yo}U_{fo}
A_{yo}G_{yo}C_{yo}U_{yo}U_{ys}AₑsAₑ;

wherein
A=an adenine nucleobase,
C=a cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage,
s=a phosphorothioate internucleoside linkage, and
vP=a 5′ vinyl phosphonate group.

### Second Oligomeric Compound

The second modified oligonucleotide has a nucleobase sequence of (from 5′ to 3′) AAGCUACAGAAUCUAUUUATA (SEQ ID NO: 1013), wherein each of nucleosides 1, 2, 20 and 21 (from 5′ to 3′) comprises a 2′-MOE modified sugar moiety, each of nucleosides 10 and 11 (from 5′ to 3′) comprises a 2′-fluoro modified sugar moiety, and each of nucleosides 3-9 and 12-19 (from 5′ to 3′) comprises a 2′-OMe modified sugar moiety, wherein each of the internucleoside linkages linking nucleosides 1 to 2, 2 to 3, 19 to 20, and 20 to 21 (from 5′ to 3′) is a phosphorothioate internucleoside linkage and each of the internucleoside linkages linking nucleosides 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, and 18 to 19 (from 5′ to 3′) is a phosphodiester linkage.

The second oligomeric compound is represented by the following chemical notation:

(SEQ ID NO: 1029)
AₑₛAₑₛG_{yo}C_{yo}U_{yo}A_{yo}C_{yo}A_{yo}G_{yo}A_{fo}A_{fo}U_{yo}C_{yo}U_{yo}A_{yo}U_{yo}
U_{yo}U_{yo}A_{ys}TₑₛAₑ;

wherein:
A=an adenine nucleobase,
C=a cytosine nucleobase,
G=a guanine nucleobase,
T=a thymine nucleobase,
U=a uracil nucleobase,
e=a 2′-MOE sugar moiety,
f=a 2′-fluoro sugar moiety,
y=a 2′-OMe sugar moiety,
o=a phosphodiester internucleoside linkage, and
s=a phosphorothioate internucleoside linkage.

In particular embodiments, such an oligomeric duplex is represented by the following chemical structure:

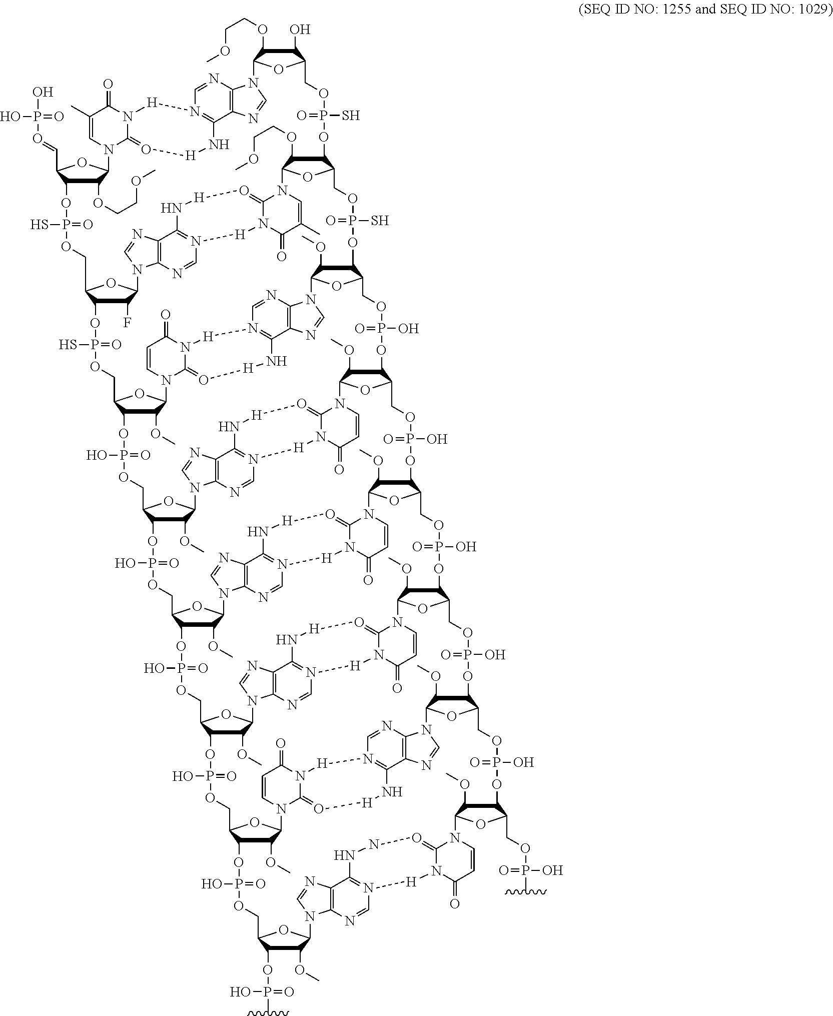

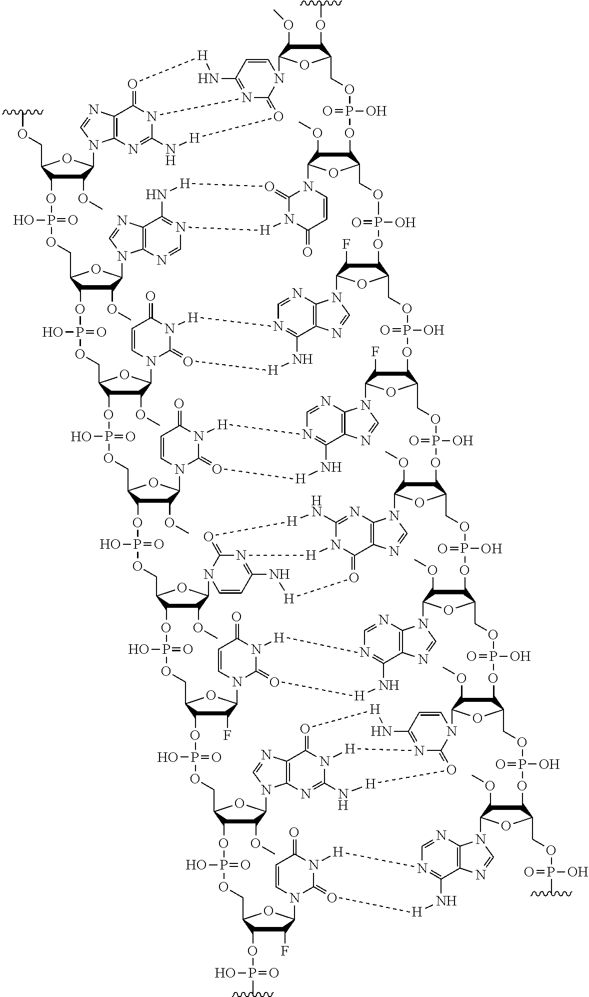

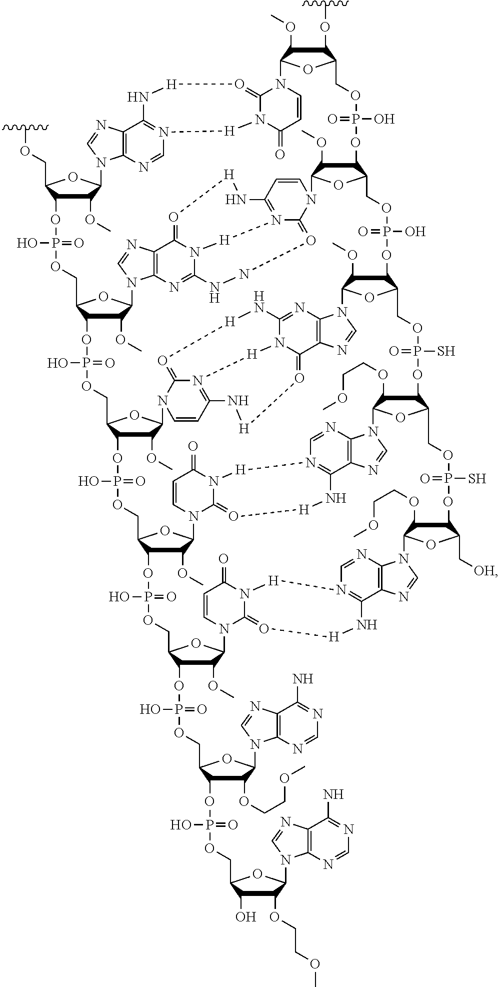

In certain embodiments, such an oligomeric duplex is in the form of an anion or a salt thereof, for example, a sodium salt. In certain embodiments, the oligomeric duplex is in anionic form in a solution. In certain embodiments, the oligomeric duplex is a sodium salt or a potassium salt.

The following chemical structure represents such an oligomeric duplex in sodium solution:

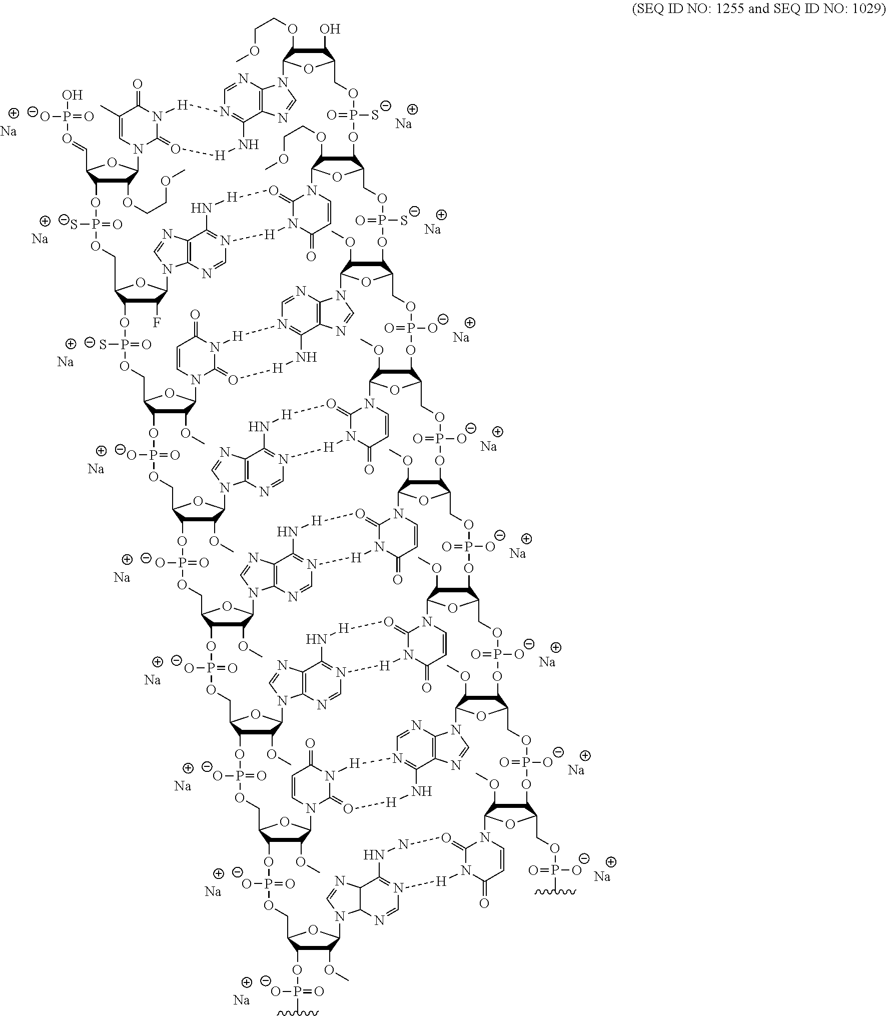

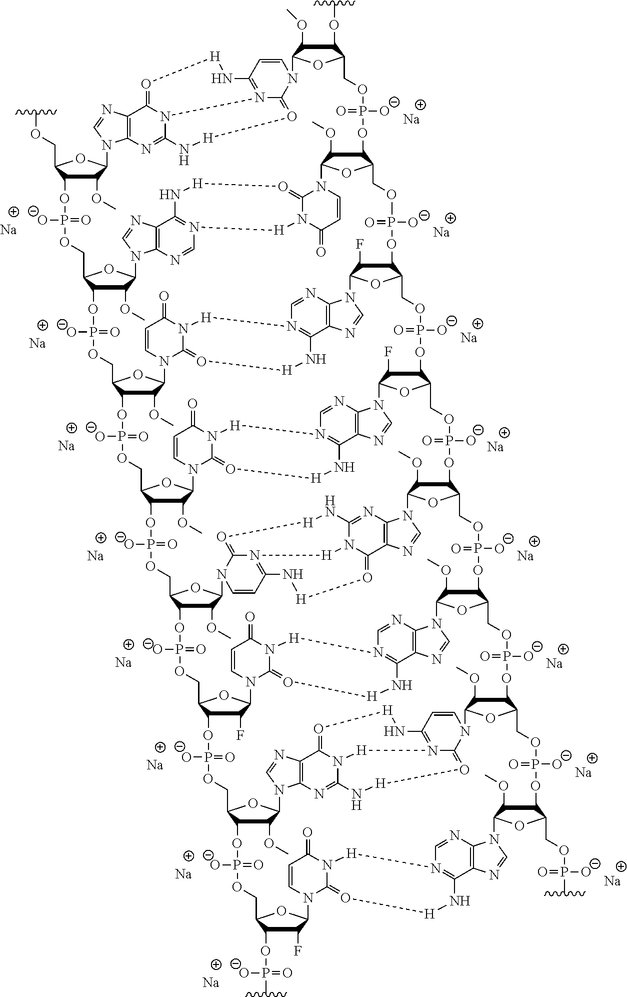

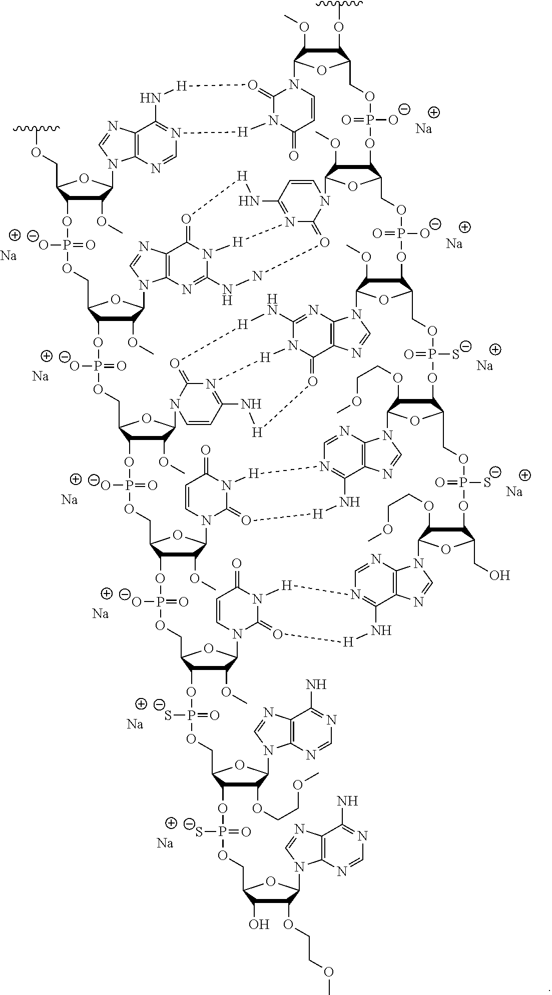

### Nonlimiting Disclosure and Incorporation by Reference

Each of the literature and patent publications listed herein is incorporated by reference in its entirety.

While certain compounds, compositions and methods described herein have been described with specificity in accordance with certain embodiments, the following examples serve only to illustrate the compounds described herein and are not intended to limit the same. Each of the references, GenBank accession numbers, ENSEMBL identifiers, and the like recited in the present application is incorporated herein by reference in its entirety.

The sequence listing accompanying this filing identifies each sequence as either “RNA” or “DNA” as required; however, one of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (5-methyl uracil) in place of an uracil of RNA).

Herein, the description of compounds as having “the nucleobase sequence of” a SEQ ID NO. describes only the nucleobase sequence. Accordingly, absent additional description, such description of compounds by reference to a nucleobase sequence of a SEQ ID NO. does not limit sugar or internucleoside linkage modifications or presence or absence of additional substituents such as a conjugate group. Herein, the description of compounds by chemical notation (subscripts and/or superscripts to indicate chemical modifications) without reference to a specific Compound No. include only each noted modification, but may include additional substituents, such as a conjugate group, unless otherwise indicated. For example, the chemical notation of “AₑₛTₖₒ^{m}C_{ez}G_{ds}C_{d}” indicates a compound wherein the first nucleoside comprises a 2′-MOE sugar moiety (indicated by the “e” subscript) and an unmodified adenine nucleobase linked to the second nucleoside via a phosphorothioate linkage (indicated by the “s” subscript); the second nucleoside comprises a cEt sugar moiety (indicated by the “k” subscript) and an unmodified thymine nucleobase linked to the third nucleoside via a phosphodiester linkage (indicated by the “o” subscript); the third nucleoside comprises a 2′-MOE sugar moiety and a 5-methyl modified cytosine nucleobase (indicated by the “m” superscript) linked to the fourth nucleoside via a mesyl phosphoramidate linkage (indicated by the “z” subscript); the fourth nucleoside comprises a 2′-deoxy sugar moiety (indicated by the “d” subscript) and an unmodified guanine nucleobase linked to the fifth nucleoside with a phosphorothioate linkage; and the fifth nucleoside comprises a 2′-deoxy sugar moiety and an unmodified cytosine nucleobase; and the compound may include additional substituents, such as a conjugate group. Herein, where a specific compound (e.g., with reference to a Compound No.) is described (as in the examples) by chemical notation, each nucleobase, sugar, and internucleoside linkage of such specific compound is modified only as indicated. Accordingly, in the context of a description of a specific compound having a particular Compound No., “AₑₛTₖₒ^{m}C_{ez}G_{ds}C_{d}” indicates a compound wherein the nucleosides comprise the indicated sugar moieties and nucleobases and are linked by the indicated internucleoside linkages and the compound does not include additional substituents unless specifically indicated otherwise.

Herein, sugar, internucleoside linkage, and nucleobase modifications may be indicated within a nucleotide or nucleobase sequence (e.g., by superscript or subscript, as shown above) or may be indicated in text accompanying a sequence (e.g., in separate text that appears within or above or below a table of compounds).

Where a specific compound is described herein by way of a drawn chemical structure, each nucleobase, sugar, and internucleoside linkage of such a specific compound includes only the modifications indicated in the drawn chemical structure. One of skill will appreciate, however, that drawn compounds may exist in equilibrium between tautomeric forms and/or as salts in equilibrium with protonated or ionic forms. Drawn structures are intended to capture all such forms of such compounds. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXEMPLIFICATION

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example A: Oligomeric Compounds that Target Human PLN

Oligomeric compounds comprising antisense oligonucleotides complementary to a human PLN nucleic acid and sense oligonucleotides complementary to the antisense oligonucleotides were previously described in International publication no. WO2022173976, filed Feb. 10, 2022, published Aug. 18, 2022, the entire contents of which is incorporated herein by reference. Oligomeric compounds were described having an antisense oligonucleotide 23 nucleosides in length; a sugar motif (from 5′ to 3′) of: yfyfyfyfyfyfyfyfyfyfyyy, wherein each “y” represents a 2′-OMe sugar moiety, and each “f” represents a 2′-fluoro sugar moiety; and has an internucleoside linkage motif (from 5′ to 3′) of: ssooooooooooooooooooss, wherein each “o” represents a phosphodiester internucleoside linkage, and each “s” represents a phosphorothioate internucleoside linkage. Sense oligonucleotide in each case is 21 nucleosides in length; has a sugar motif (from 5′ to 3′) of: fyfyfyfyfyfyfyfyfyfyf, wherein each “y” represents a 2′-OMe sugar moiety, and each “f” represents a 2′-fluoro sugar moiety; and has an internucleoside linkage motif (from 5′ to 3′) of: ssooooooooooooooooss, wherein each “o” represents a phosphodiester internucleoside linkage, and each “s” represents a phosphorothioate intenucleoside linkage. Each antisense oligonucleotide having the nucleobase sequence in Table A is complementary to the target nucleic acid (PLN), and each sense oligonucleotide having the nucleobase sequence in Table A is complementary to the first ofthe 21 nucleosides of the antisense oligonucleotide (from 5′ to 3′) wherein the last two 3′-nucleosides ofthe antisense oligonucleotides are not paired with the sense oligonucleotide (are overhanging nucleosides). “Start site” indicates the 5′-most nucleoside to which the antisense oligonucleotide is complementary in the human PLN sequence. “Stop site” indicates the 3′-most nucleoside to which the antisense oligonucleotide is complementary in the human PLN sequence. Each modified antisense oligonucleotide listed in Table A is 100% complementary to SEQ ID NO: 1 (GENBANK Accession No. NM_002667.4), with the exception of Compound No. 1564340 which is 10000 complementary to SEQ ID NO: 2 (GENBANK Accession No. NC_000006.12, truncated from nucleosides 118545001 to 118565000) from nucleosides 16694 to 16716.

TABLE A
Oligomeric compounds targeting human PLN

| | | | | SEQ ID | | | | |
|---|---|---|---|---|---|---|---|---|
| | | | | NO: 1 | SEQ ID | | | |
| | | | | Anti- | NO: 1 | | | |
| | Anti- | Antisense | SEQ | sense | Anti- | | Sense | SEQ |
| Compound | sense | Sequence | ID | Start | sense | Sense | Sequence | ID |
| Number | ID | (5′ to 3′) | NO. | Site | Stop Site | ID | (5′ to 3′) | NO. |
| 1563877 | 1563889 | UCAUCACAGUAU | 3 | 94 | 116 | 1563885 | CAAUACUCUAU | 315 |
| | | AGAGUAUUGUG | | | | | ACUGUGAUGA | |
| 1563878 | 1563891 | GGUGUUUAGCUG | 4 | 54 | 76 | 1563883 | AAAACUCCCCA | 316 |
| | | GGGAGUUUUCU | | | | | GCUAAACACC | |
| 1563874 | 1563886 | UCUGACUCUGUC | 5 | 34 | 56 | 1563880 | AAACUGGGUGA | 317 |
| | | ACCCAGUUUAU | | | | | CAGAGUCAGA | |
| 1563875 | 1563887 | GGUAGCCUUGGC | 6 | 114 | 136 | 1563881 | AUCACAGCUGC | 318 |
| | | AGCUGUGAUCA | | | | | CAAGGCUACC | |
| 1563876 | 1563890 | GUGUUGUAUGAA | 7 | 74 | 96 | 1563882 | CCGUAAGACUU | 319 |
| | | GUCUUACGGGU | | | | | CAUACAACAC | |
| 1563879 | 1563888 | GAGAUAACUGUC | 8 | 134 | 156 | 1563884 | CUAAAAGAAGA | 320 |
| | | UUCUUUUAGGU | | | | | CAGUUAUCUC | |
| 1563894 | 1563905 | UUUCUCCAUGAU | 9 | 214 | 236 | 1563900 | CCUGCUGGUAU | 321 |
| | | ACCAGCAGGAC | | | | | CAUGGAGAAA | |
| 1563897 | 1563909 | AAAAGCUGGCAG | 10 | 154 | 176 | 1563903 | CAUAUUUGGCU | 322 |
| | | CCAAAUAUGAG | | | | | GCCAGCUUUU | |
| 1563892 | 1563906 | AAGUGGUCGAGA | 11 | 174 | 196 | 1563899 | UUAUCUUUCUC | 323 |
| | | GAAAGAUAAAA | | | | | UCGACCACUU | |
| 1563896 | 1563908 | GACAGGAAGUCU | 12 | 194 | 216 | 1563902 | UAAAACUUCAG | 324 |
| | | GAAGUUUUAAG | | | | | ACUUCCUGUC | |
| 1563893 | 1563907 | UUGAGGCUCUUC | 13 | 254 | 276 | 1563901 | CAGCUAUAAGA | 325 |
| | | UUAUAGCUGAG | | | | | AGAGCCUCAA | |
| 1563895 | 1563904 | GAGCGAGUGAGG | 14 | 234 | 256 | 1563898 | AGUCCAAUACC | 326 |
| | | UAUUGGACUUU | | | | | UCACUCGCUC | |
| 1563910 | 1563925 | UUGUUGAGGCAU | 15 | 274 | 296 | 1563919 | ACCAUUGAAAU | 327 |
| | | UUCAAUGGUUG | | | | | GCCUCAACAA | |
| 1563915 | 1563926 | UUCUGUAGCUUU | 16 | 294 | 316 | 1563920 | AGCACGUCAAA | 328 |
| | | UGACGUGCUUG | | | | | AGCUACAGAA | |
| 1563911 | 1563924 | UAGCAGAACUUC | 17 | 374 | 396 | 1563918 | UGCUUCUCUGA | 329 |
| | | AGAGAAGCAUC | | | | | AGUUCUGCUA | |
| 1563913 | 1563922 | AUCACGAUGAUA | 18 | 354 | 376 | 1563917 | GCUGAUCUGUA | 330 |
| | | CAGAUCAGCAA | | | | | UCAUCGUGAU | |
| 1563914 | 1563927 | GACAGAAAUUGA | 19 | 314 | 336 | 1563921 | AUCUAUUUAUC | 331 |
| | | UAAAUAGAUUC | | | | | AAUUUCUGUC | |
| 1563912 | 1563923 | CAAGAGACAUAU | 20 | 334 | 356 | 1563916 | CUCAUCUUAAU | 332 |
| | | UAAGAUGAGAC | | | | | AUGUCUCUUG | |
| 1563928 | 1563945 | AGCUGCAGAUCU | 21 | 394 | 416 | 1563939 | ACAACCUCUAG | 333 |
| | | AGAGGUUGUAG | | | | | AUCUGCAGCU | |
| 1563931 | 1563942 | CUGUUAUACAAU | 22 | 454 | 476 | 1563934 | GAAAACAAUAU | 334 |
| | | AUUGUUUUCCU | | | | | UGUAUAACAG | |
| 1563933 | 1563940 | GAUUUUAAGCUG | 23 | 414 | 436 | 1563936 | UUGCCACAUCA | 335 |
| | | AUGUGGCAAGC | | | | | GCUUAAAAUC | |
| 1563932 | 1563943 | CCUGUCUGCAUG | 24 | 434 | 456 | 1563937 | CUGUCAUCCCA | 336 |
| | | GGAUGACAGAU | | | | | UGCAGACAGG | |
| 1563930 | 1563941 | UCUUCUACUCAG | 25 | 474 | 496 | 1563935 | GACCACUUCCU | 337 |
| | | GAAGUGGUCUG | | | | | GAGUAGAAGA | |
| 1563929 | 1563944 | UGACCUUUUCAC | 26 | 494 | 516 | 1563938 | AGUUUCUUUGU | 338 |
| | | AAAGAAACUCU | | | | | GAAAAGGUCA | |
| 1563947 | 1563960 | AUCAACAGUUGC | 27 | 614 | 636 | 1563954 | GUAUAAAAUGC | 339 |
| | | AUUUUAUACAC | | | | | AACUGUUGAU | |
| 1563948 | 1563958 | CACUUAUUUUGA | 28 | 594 | 616 | 1563953 | AAAUUAACUUC | 340 |
| | | AGUUAAUUUUU | | | | | AAAAUAAGUG | |
| 1563951 | 1563962 | AUAAGUUUUAGU | 29 | 514 | 536 | 1563956 | AAGAUUAAGAC | 341 |
| | | CUUAAUCUUGA | | | | | UAAAACUUAU | |
| 1563950 | 1563963 | CAUGUUUACAAG | 30 | 554 | 576 | 1563957 | CUGUUGGAUCU | 342 |
| | | AUCCAACAGAU | | | | | UGUAAACAUG | |
| 1563946 | 1563959 | GAUGAAUACAUA | 31 | 534 | 556 | 1563955 | UUGUUACCAUA | 343 |
| | | UGGUAACAAUA | | | | | UGUAUUCAUC | |
| 1563949 | 1563961 | UUUGAAAAUAAA | 32 | 574 | 596 | 1563952 | GAAAAGGGCUU | 344 |
| | | GCCCUUUUCAU | | | | | UAUUUUCAAA | |
| 1563964 | 1563976 | ACUAAACUCUUC | 33 | 674 | 696 | 1563973 | UCUGAAGAUGA | 345 |
| | | AUCUUCAGAAA | | | | | AGAGUUUAGU | |
| 1563965 | 1563977 | CAAAAGAGUAAA | 34 | 734 | 756 | 1563972 | AGCAUUAUUUU | 346 |
| | | AAUAAUGCUUU | | | | | UACUCUUUUG | |
| 1563969 | 1563980 | UUGUGAGCCAUG | 35 | 634 | 656 | 1563974 | UUUCCUCAACA | 347 |
| | | UUGAGGAAAUC | | | | | UGGCUCACAA | |
| 1563967 | 1563975 | GUUGGCAGUGCA | 36 | 694 | 716 | 1563970 | UUUUAAAACUG | 348 |
| | | GUUUUAAAACU | | | | | CACUGCCAAC | |
| 1563966 | 1563979 | UUUAUAUAUGAA | 37 | 714 | 736 | 1563971 | CAAGUUCACUU | 349 |
| | | GUGAACUUGUU | | | | | CAUAUAUAAA | |
| 1563968 | 1563981 | AAAAGAUUUGGG | 38 | 654 | 676 | 1563978 | AAUUUCUAUCC | 350 |
| | | AUAGAAAUUUG | | | | | CAAAUCUUUU | |
| 1563985 | 1563996 | UACUUUGAUACU | 39 | 814 | 836 | 1563988 | UCUUCACCAAG | 351 |
| | | UGGUGAAGACC | | | | | UAUCAAAGUA | |
| 1563983 | 1563994 | UGACACUUCAUU | 40 | 834 | 856 | 1563990 | AAUAACACAAA | 352 |
| | | UGUGUUAUUAC | | | | | UGAAGUGUCA | |
| 1563982 | 1563997 | AAUAUAAAUUAU | 41 | 754 | 776 | 1563991 | GAGGUGAAUAU | 353 |
| | | AUUCACCUCAA | | | | | AAUUUAUAUU | |
| 1563986 | 1563998 | ACCUGAAAAAUA | 42 | 794 | 816 | 1563993 | AAUACUAAGUA | 354 |
| | | CUUAGUAUUAA | | | | | UUUUUCAGGU | |
| 1563987 | 1563999 | UAAAGAAGCUUU | 43 | 774 | 796 | 1563992 | UACAAUGUAAA | 355 |
| | | UACAUUGUAAU | | | | | AGCUUCUUUA | |
| 1563984 | 1563995 | GUUAUUACUUUG | 44 | 819 | 841 | 1563989 | ACCAAGUAUCA | 356 |
| | | AUACUUGGUGA | | | | | AAGUAAUAAC | |
| 1564003 | 1564015 | UGUAGAUUCUGA | 45 | 914 | 936 | 1564006 | UAGUAACUAUC | 357 |
| | | UAGUUACUACA | | | | | AGAAUCUACA | |
| 1564000 | 1564012 | CAGUGGACUAUU | 46 | 854 | 876 | 1564009 | AUUAUUCAAAA | 358 |
| | | UUGAAUAAUGA | | | | | UAGUCCACUG | |
| 1564005 | 1564017 | AGAUAACAGAUG | 47 | 874 | 896 | 1564010 | GACUCCUCACA | 359 |
| | | UGAGGAGUCAG | | | | | UCUGUUAUCU | |
| 1564002 | 1564013 | AUACAAUUUCUG | 48 | 934 | 956 | 1564007 | AUUCUAAAACA | 360 |
| | | UUUUAGAAUGU | | | | | GAAAUUGUAU | |
| 1564004 | 1564016 | ACAAAUAGUUCU | 49 | 894 | 916 | 1564011 | UUAUUAUAAAG | 361 |
| | | UUAUAAUAAGA | | | | | AACUAUUUGU | |
| 1564001 | 1564014 | GUUAAUGUGGCA | 50 | 954 | 976 | 1564008 | UUUUUUCUAUG | 362 |
| | | UAGAAAAAAUA | | | | | CCACAUUAAC | |
| 1564021 | 1564029 | CUUACUUUUCCA | 51 | 994 | 1016 | 1564025 | AAUCAAGUAUG | 363 |
| | | UACUUGAUUCU | | | | | GAAAAGUAAG | |
| 1564022 | 1564034 | UUAUUAUGUAAG | 52 | 1014 | 1036 | 1564028 | GGCCAUACUCU | 364 |
| | | AGUAUGGCCUU | | | | | UACAUAAUAA | |
| 1564018 | 1564030 | UCUCAUCAACUU | 53 | 974 | 996 | 1564026 | CAUCUUUUAAA | 365 |
| | | UAAAAGAUGUU | | | | | GUUGAUGAGA | |
| 1564023 | 1564035 | CAUACUUGAUUC | 54 | 984 | 1006 | 1564033 | AGUUGAUGAGA | 366 |
| | | UCAUCAACUUU | | | | | AUCAAGUAUG | |
| 1564020 | 1564031 | AAAAUUACUUAA | 55 | 1034 | 1056 | 1564024 | AAAUUCCUUUU | 367 |
| | | AAGGAAUUUUA | | | | | AAGUAAUUUU | |
| 1564019 | 1564032 | GAAUUCUGUGAU | 56 | 1054 | 1076 | 1564027 | UUUCAAAGAAU | 368 |
| | | UCUUUGAAAAA | | | | | CACAGAAUUC | |
| 1564036 | 1564051 | UAUGUCUUAGAA | 57 | 1094 | 1116 | 1564045 | AUAAAUCUGUU | 369 |
| | | CAGAUUUAUGA | | | | | CUAAGACAUA | |
| 1564038 | 1564047 | AUUAGUGAUAUG | 58 | 1154 | 1176 | 1564043 | GAGAUUAGUCA | 370 |
| | | ACUAAUCUCAC | | | | | UAUCACUAAU | |
| 1564040 | 1564053 | CACUGUCACAUA | 59 | 1134 | 1156 | 1564048 | GGUGGUUAAUA | 371 |
| | | UUAACCACCAG | | | | | UGUGACAGUG | |
| 1564037 | 1564050 | UUAGAUUCUGUU | 60 | 1174 | 1196 | 1564044 | UAUACUAACAA | 372 |
| | | GUUAGUAUAUU | | | | | CAGAAUCUAA | |
| 1564039 | 1564049 | CAGUUCUCAUCU | 61 | 1114 | 1136 | 1564042 | AUGAUCAACAG | 373 |
| | | GUUGAUCAUAU | | | | | AUGAGAACUG | |
| 1564041 | 1564052 | UGAUUUACCUAC | 62 | 1074 | 1096 | 1564046 | CUAGUACAUGU | 374 |
| | | AUGUACUAGAA | | | | | AGGUAAAUCA | |
| 1564058 | 1564071 | UAGUAUGGUAAG | 63 | 1234 | 1256 | 1564065 | GUUACCUAGCU | 375 |
| | | CUAGGUAACUC | | | | | UACCAUACUA | |
| 1564054 | 1564067 | UACAGUGCCUUA | 64 | 1194 | 1216 | 1564063 | AUCUUCAUUUA | 376 |
| | | AAUGAAGAUUA | | | | | AGGCACUGUA | |
| 1564059 | 1564070 | CUCUAGCUCAGA | 65 | 1214 | 1236 | 1564064 | AGUGAAUUAUC | 377 |
| | | UAAUUCACUAC | | | | | UGAGCUAGAG | |
| 1564057 | 1564069 | GAUAUAGUAUGG | 66 | 1238 | 1260 | 1564061 | CCUAGCUUACC | 378 |
| | | UAAGCUAGGUA | | | | | AUACUAUAUC | |
| 1564055 | 1564068 | UGAUUCCAAAGA | 67 | 1248 | 1270 | 1564062 | CAUACUAUAUC | 379 |
| | | UAUAGUAUGGU | | | | | UUUGGAAUCA | |
| 1564056 | 1564066 | CCAAAGAUAUAG | 68 | 1243 | 1265 | 1564060 | CUUACCAUACU | 380 |
| | | UAUGGUAAGCU | | | | | AUAUCUUUGG | |
| 1564074 | 1564081 | CUGCAUUGGAUG | 69 | 1314 | 1336 | 1564078 | CCAGCCUAACA | 381 |
| | | UUAGGCUGGAA | | | | | UCCAAUGCAG | |
| 1564072 | 1564082 | GUUUCAUGAUUC | 70 | 1254 | 1276 | 1564077 | AUAUCUUUGGA | 382 |
| | | CAAAGAUAUAG | | | | | AUCAUGAAAC | |
| 1564084 | 1564088 | AAAUCUUUUAUU | 71 | 1334 | 1356 | 1564086 | GGCAAGGAAAA | 383 |
| | | UUCCUUGCCUG | | | | | UAAAAGAUUU | |
| 1564073 | 1564080 | GAAUGGAAGACA | 72 | 1294 | 1316 | 1564076 | UUUGCAGGUUG | 384 |
| | | ACCUGCAAAAU | | | | | UCUUCCAUUC | |
| 1564085 | 1564089 | AUAUAUUUUUCU | 73 | 1354 | 1376 | 1564087 | UCCAGUGACAG | 385 |
| | | GUCACUGGAAA | | | | | AAAAAUAUAU | |
| 1564075 | 1564083 | AAUCAUUCUGAA | 74 | 1274 | 1296 | 1564079 | CCUUAAGACUU | 386 |
| | | GUCUUAAGGUU | | | | | CAGAAUGAUU | |
| 1564090 | 1564104 | UUUAAAAAAUAC | 75 | 1374 | 1396 | 1564101 | UUAUCUCAAGU | 387 |
| | | UUGAGAUAAUA | | | | | AUUUUUUAAA | |
| 1564093 | 1564105 | CUAAUAAUUAGU | 76 | 1414 | 1436 | 1564100 | CAAAUAUUAAC | 388 |
| | | UAAUAUUUGGA | | | | | UAAUUAUUAG | |
| 1564094 | 1564102 | GGAGAGAGAAUU | 77 | 1394 | 1416 | 1564097 | AAAUAUAUGAA | 389 |
| | | CAUAUAUUUUU | | | | | UUCUCUCUCC | |
| 1564092 | 1564103 | AUGUAAUAGAUG | 78 | 1454 | 1476 | 1564099 | UUGUUGGCCCA | 390 |
| | | GGCCAACAAGU | | | | | UCUAUUACAU | |
| 1564091 | 1564107 | UGUUCAAGGGUC | 79 | 1474 | 1496 | 1564096 | UCUACAGCUGA | 391 |
| | | AGCUGUAGAUG | | | | | CCCUUGAACA | |
| 1564095 | 1564106 | AGUUCAUUUCAA | 80 | 1434 | 1456 | 1564098 | GAUUAUAUUUU | 392 |
| | | AAUAUAAUCUA | | | | | GAAAUGAACU | |
| 1564113 | 1564122 | GUCAGCUCCCCU | 81 | 1494 | 1516 | 1564118 | AUGGGGGUUAG | 393 |
| | | AACCCCCAUGU | | | | | GGGAGCUGAC | |
| 1564111 | 1564124 | UAGUUAAGAUUU | 82 | 1522 | 1544 | 1564116 | GGGUCCGCAAA | 394 |
| | | UGCGGACCCAC | | | | | AUCUUAACUA | |
| 1564110 | 1564120 | AUUUACUGUUUA | 83 | 1574 | 1596 | 1564114 | CUGAUAACAUA | 395 |
| | | UGUUAUCAGUA | | | | | AACAGUAAAU | |
| 1564109 | 1564121 | GUAAGGUUUAUG | 84 | 1554 | 1576 | 1564115 | ACUAUUGACCA | 396 |
| | | GUCAAUAGUAG | | | | | UAAACCUUAC | |
| 1564112 | 1564123 | UAGGCUAUUAGG | 85 | 1534 | 1556 | 1564119 | UCUUAACUACC | 397 |
| | | UAGUUAAGAUU | | | | | UAAUAGCCUA | |
| 1564108 | 1564125 | AUUUUGCGGACC | 86 | 1514 | 1536 | 1564117 | CAAUUCGUGGG | 398 |
| | | CACGAAUUGUC | | | | | UCCGCAAAAU | |
| 1564127 | 1564139 | UAUUUUCUCUUU | 87 | 1674 | 1696 | 1564134 | AAUCAUAAGAA | 399 |
| | | CUUAUGAUUUU | | | | | AGAGAAAAUA | |
| 1564129 | 1564142 | UAGCUUACUUUA | 88 | 1634 | 1656 | 1564137 | UUCCUACAAUA | 400 |
| | | UUGUAGGAAUA | | | | | AAGUAAGCUA | |
| 1564128 | 1564140 | CAUUUAAUGAAU | 89 | 1694 | 1716 | 1564132 | AUAUUUACUAU | 401 |
| | | AGUAAAUAUAU | | | | | UCAUUAAAUG | |
| 1564131 | 1564138 | AACACGCAAAAU | 90 | 1594 | 1616 | 1564133 | UUAACACAUAU | 402 |
| | | AUGUGUUAAUU | | | | | UUUGCGUGUU | |
| 1564130 | 1564141 | UUUCUAAAUAAC | 91 | 1654 | 1676 | 1564136 | AGAGAAAAUGU | 403 |
| | | AUUUUCUCUAG | | | | | UAUUUAGAAA | |
| 1564126 | 1564143 | AUAUAGUGUAUA | 92 | 1614 | 1636 | 1564135 | UAUAUGUAUUA | 404 |
| | | AUACAUAUAAC | | | | | UACACUAUAU | |
| 1564146 | 1564157 | CUCCUCUGCCUA | 93 | 1754 | 1776 | 1564152 | CACAUUGAGUA | 405 |
| | | CUCAAUGUGAA | | | | | GGCAGAGGAG | |
| 1564145 | 1564159 | CCCCACACCCCU | 94 | 1814 | 1836 | 1564153 | UCUUGUCUUAG | 406 |
| | | AAGACAAGACU | | | | | GGGUGUGGGG | |
| 1564147 | 1564158 | UCUUCCUCCCCA | 95 | 1774 | 1796 | 1564150 | GGAGAAAGAUG | 407 |
| | | UCUUUCUCCUC | | | | | GGGAGGAAGA | |
| 1564149 | 1564160 | CUUUUAUGUUGA | 96 | 1714 | 1736 | 1564155 | GGAAGUGGGUC | 408 |
| | | CCCACUUCCAU | | | | | AACAUAAAAG | |
| 1564148 | 1564161 | GAAGACAAUGAG | 97 | 1734 | 1756 | 1564154 | GUCUUCAUUCU | 409 |
| | | AAUGAAGACUU | | | | | CAUUGUCUUC | |
| 1564144 | 1564156 | ACUGCAAGACCA | 98 | 1794 | 1816 | 1564151 | AGAAGGCGUUG | 410 |
| | | ACGCCUUCUCU | | | | | GUCUUGCAGU | |
| 1564167 | 1564178 | UGAAUAUUCUUU | 99 | 1834 | 1856 | 1564169 | GAGUGGGGGAA | 411 |
| | | CCCCCACUCCC | | | | | AGAAUAUUCA | |
| 1564162 | 1564174 | UGCAAGGGUCCA | 100 | 1854 | 1876 | 1564170 | AUGUAUAAGUG | 412 |
| | | CUUAUACAUGA | | | | | GACCCUUGCA | |
| 1564166 | 1564175 | AUCCUAUUACAG | 101 | 1894 | 1916 | 1564168 | AAGGGUCAACU | 413 |
| | | UUGACCCUUGA | | | | | GUAAUAGGAU | |
| 1564163 | 1564176 | AGAGGAAGAAAA | 102 | 1914 | 1936 | 1564172 | UAUAGCUAUUU | 414 |
| | | AUAGCUAUAUC | | | | | UUCUUCCUCU | |
| 1564164 | 1564177 | AUGCUUACCAUU | 103 | 1934 | 1956 | 1564173 | UAUCAACCAAA | 415 |
| | | UGGUUGAUAGA | | | | | UGGUAAGCAU | |
| 1564165 | 1564179 | UGAACAACAAGG | 104 | 1874 | 1896 | 1564171 | AAUUCAAGCCC | 416 |
| | | GCUUGAAUUGC | | | | | UUGUUGUUCA | |
| 1564184 | 1564195 | AAAUUUAUUCAU | 105 | 2014 | 2036 | 1564188 | AUUUUCUUGAU | 417 |
| | | CAAGAAAAUAA | | | | | GAAUAAAUUU | |
| 1564181 | 1564194 | UUAUUGGAAGAU | 106 | 2054 | 2076 | 1564189 | UUUCAGAACAU | 418 |
| | | GUUCUGAAAUG | | | | | CUUCCAAUAA | |
| 1564180 | 1564192 | GGAUCUAUAAUU | 107 | 1974 | 1996 | 1564191 | ACUGAGCUAAA | 419 |
| | | UAGCUCAGUAG | | | | | UUAUAGAUCC | |
| 1564183 | 1564193 | UAAUUAUAAAUA | 108 | 1994 | 2016 | 1564190 | CAGCUAUGCUA | 420 |
| | | GCAUAGCUGGA | | | | | UUUAUAAUUA | |
| 1564182 | 1564197 | AUGGUCAGAGGA | 109 | 2034 | 2056 | 1564186 | UUCAAUUUCUC | 421 |
| | | GAAAUUGAAAA | | | | | CUCUGACCAU | |
| 1564185 | 1564196 | UAGAGUGGACUG | 110 | 1954 | 1976 | 1564187 | UCUAUUUUGCA | 422 |
| | | CAAAAUAGAUG | | | | | GUCCACUCUA | |
| 1564202 | 1564215 | UUACCAAAGUUU | 111 | 2114 | 2136 | 1564205 | AUAUUCACCAA | 423 |
| | | GGUGAAUAUAU | | | | | ACUUUGGUAA | |
| 1564199 | 1564210 | GAUUAUGCAGUA | 112 | 2174 | 2196 | 1564204 | UUACACCUAUA | 424 |
| | | UAGGUGUAAAC | | | | | CUGCAUAAUC | |
| 1564198 | 1564212 | UAUUUUCCAGCA | 113 | 2094 | 2116 | 1564209 | AAAAUUGAGUG | 425 |
| | | CUCAAUUUUAC | | | | | CUGGAAAAUA | |
| 1564200 | 1564214 | AACUAUUUUAGA | 114 | 2154 | 2176 | 1564208 | AAAAUUAAGUC | 426 |
| | | CUUAAUUUUAA | | | | | UAAAAUAGUU | |
| 1564201 | 1564211 | UAAACUUUAGUC | 115 | 2134 | 2156 | 1564206 | AUUUAAGUUGA | 427 |
| | | AACUUAAAUUA | | | | | CUAAAGUUUA | |
| 1564203 | 1564213 | UACUUCAGUUGU | 116 | 2074 | 2096 | 1564207 | ACUCAUAAAAC | 428 |
| | | UUUAUGAGUUA | | | | | AACUGAAGUA | |
| 1564216 | 1564228 | AACUGAAAUUAA | 117 | 2194 | 2216 | 1564222 | CCAACAAUUUU | 429 |
| | | AAUUGUUGGAU | | | | | AAUUUCAGUU | |
| 1564221 | 1564229 | UAUAUUAGUAAC | 118 | 2214 | 2236 | 1564227 | UGAAGACAUGU | 430 |
| | | AUGUCUUCAAC | | | | | UACUAAUAUA | |
| 1564217 | 1564231 | UAGUAACCAUGU | 119 | 2274 | 2296 | 1564224 | UCUUCUAAAAC | 431 |
| | | UUUAGAAGAUA | | | | | AUGGUUACUA | |
| 1564220 | 1564233 | AUAUGGUUAAUU | 120 | 2254 | 2276 | 1564223 | AGGAUGUGUAA | 432 |
| | | ACACAUCCUCU | | | | | UUAACCAUAU | |
| 1564218 | 1564232 | AUUGAUGUUACA | 121 | 2294 | 2316 | 1564225 | AAAAGAAUAUG | 433 |
| | | UAUUCUUUUAG | | | | | UAACAUCAAU | |
| 1564219 | 1564230 | UCUACUCUUUUA | 122 | 2234 | 2256 | 1564226 | AACUAUUAUUA | 434 |
| | | AUAAUAGUUAU | | | | | AAAGAGUAGA | |
| 1564235 | 1564247 | UAUCCCUGAACC | 123 | 2414 | 2436 | 1564241 | AAAGUGUUUGG | 435 |
| | | AAACACUUUCC | | | | | UUCAGGGAUA | |
| 1564237 | 1564248 | UCUCUCAUGGGU | 124 | 2374 | 2396 | 1564242 | AUUUUCUGAAC | 436 |
| | | UCAGAAAAUUU | | | | | CCAUGAGAGA | |
| 1564234 | 1564249 | UAUUGAGUUAGC | 125 | 2334 | 2356 | 1564244 | ACAAGUGUUGC | 437 |
| | | AACACUUGUGU | | | | | UAACUCAAUA | |
| 1564238 | 1564250 | UGUAAGAAACCA | 126 | 2314 | 2336 | 1564243 | UAUUGACCUUG | 438 |
| | | AGGUCAAUAUU | | | | | GUUUCUUACA | |
| 1564256 | 1564265 | GAAUUUCCAGCU | 127 | 2474 | 2496 | 1564261 | UCAUCUACAAG | 439 |
| | | UGUAGAUGAGG | | | | | CUGGAAAUUC | |
| 1564236 | 1564246 | UUUAAUAGUGUC | 128 | 2354 | 2376 | 1564240 | AGUGAAGGAGA | 440 |
| | | UCCUUCACUAU | | | | | CACUAUUAAA | |
| 1564239 | 1564251 | UCCACUCCCCAU | 129 | 2394 | 2416 | 1564245 | AUACUAGAGAU | 441 |
| | | CUCUAGUAUCU | | | | | GGGGAGUGGA | |
| 1564253 | 1564269 | ACUAAAACCAGU | 130 | 2534 | 2556 | 1564258 | ACUCACCUCAC | 442 |
| | | GAGGUGAGUGU | | | | | UGGUUUUAGU | |
| 1564257 | 1564268 | AGGCGUCACUUA | 131 | 2454 | 2476 | 1564263 | GAGAUUUCUUA | 443 |
| | | AGAAAUCUCUG | | | | | AGUGACGCCU | |
| 1564254 | 1564267 | UGUAUCACCUGU | 132 | 2514 | 2536 | 1564259 | UUAUAAACAAC | 444 |
| | | UGUUUAUAAGC | | | | | AGGUGAUACA | |
| 1564252 | 1564266 | CUGCCCUUCUGU | 133 | 2434 | 2456 | 1564260 | AUCUGAAGAAC | 445 |
| | | UCUUCAGAUAU | | | | | AGAAGGGCAG | |
| 1564255 | 1564264 | AGCUUUCUACUU | 134 | 2494 | 2516 | 1564262 | CCUAAAAACAA | 446 |
| | | GUUUUUAGGAA | | | | | GUAGAAAGCU | |
| 1564270 | 1564283 | ACUUUCUGUAUU | 135 | 2554 | 2576 | 1564280 | UAAAUUACCAA | 447 |
| | | GGUAAUUUACU | | | | | UACAGAAAGU | |
| 1564274 | 1564284 | AAUCAGUUCAAA | 136 | 2594 | 2616 | 1564278 | AGUGGAAAAUU | 448 |
| | | UUUUCCACUUG | | | | | UGAACUGAUU | |
| 1564271 | 1564285 | AAAAUAUUGUAA | 137 | 2634 | 2656 | 1564277 | CACUGUUUGUU | 449 |
| | | CAAACAGUGUA | | | | | ACAAUAUUUU | |
| 1564275 | 1564286 | GUUAUUUCUGUU | 138 | 2654 | 2676 | 1564276 | UUCUCAGUAAA | 450 |
| | | UACUGAGAAAA | | | | | CAGAAAUAAC | |
| 1564272 | 1564282 | UUGUUUUUAAGA | 139 | 2574 | 2596 | 1564279 | UAUCCCUAGUC | 451 |
| | | CUAGGGAUACU | | | | | UUAAAAACAA | |
| 1564273 | 1564287 | GUAAUCAAAGGA | 140 | 2614 | 2636 | 1564281 | UAGUCAUAUUC | 452 |
| | | AUAUGACUAAU | | | | | CUUUGAUUAC | |
| 1564292 | 1564304 | AAUGAAGAACAA | 141 | 2674 | 2696 | 1564299 | CUAAUUUUUUU | 453 |
| | | AAAAAUUAGUU | | | | | GUUCUUCAUU | |
| 1564293 | 1564305 | UAUGAUGAAGAA | 142 | 2774 | 2796 | 1564298 | AAAUAAUUAUU | 454 |
| | | UAAUUAUUUAA | | | | | CUUCAUCAUA | |
| 1564288 | 1564300 | UAAAGAGUUUAU | 143 | 2754 | 2776 | 1564294 | UAAAGUAGAAU | 455 |
| | | UCUACUUUAUA | | | | | AAACUCUUUA | |
| 1564291 | 1564302 | GAUUUUAAUUUC | 144 | 2694 | 2716 | 1564297 | UCUUUGAUAGA | 456 |
| | | UAUCAAAGAAU | | | | | AAUUAAAAUC | |
| 1564289 | 1564301 | AUAAUUUGAGUU | 145 | 2734 | 2756 | 1564296 | GAAUACUAUAA | 457 |
| | | AUAGUAUUCUG | | | | | CUCAAAUUAU | |
| 1564290 | 1564303 | CUGUAAUCCUCA | 146 | 2714 | 2736 | 1564295 | CUUAUUCUGUG | 458 |
| | | CAGAAUAAGAU | | | | | AGGAUUACAG | |
| 1564308 | 1564319 | AUUGUUUUUAGU | 147 | 2894 | 2916 | 1564316 | UUACAGGGCAC | 459 |
| | | GCCCUGUAAAU | | | | | UAAAAACAAU | |
| 1564310 | 1564320 | CUAUAGUAGAAC | 148 | 2854 | 2876 | 1564313 | CUCAAAUAUGU | 460 |
| | | AUAUUUGAGCA | | | | | UCUACUAUAG | |
| 1564307 | 1564321 | AAUUAAGAUAAG | 149 | 2874 | 2896 | 1564317 | GAAUAAGUUCU | 461 |
| | | AACUUAUUCUA | | | | | UAUCUUAAUU | |
| 1564311 | 1564323 | AUAUCUUAUUCU | 150 | 2794 | 2816 | 1564314 | AAAGUGUAAAG | 462 |
| | | UUACACUUUAU | | | | | AAUAAGAUAU | |
| 1564309 | 1564318 | GCAUUUAGUAUA | 151 | 2834 | 2856 | 1564312 | AAAAUUUAAUA | 463 |
| | | UUAAAUUUUAA | | | | | UACUAAAUGC | |
| 1564306 | 1564322 | UAAAAAUAAAUU | 152 | 2814 | 2836 | 1564315 | UAAGAAAACAA | 464 |
| | | GUUUUCUUAUA | | | | | UUUAUUUUUA | |
| 1564328 | 1564336 | UUAAACAAAUAU | 153 | 2954 | 2976 | 1564332 | UAAGAAUAUAU | 465 |
| | | AUAUUCUUAAC | | | | | AUUUGUUUAA | |
| 1564325 | 1564340 | UGAGUUUUCAAG | 154 | N/A | N/A | 1564335 | AUGAAUGUUCU | 466 |
| | | AACAUUCAUUU | | | | | UGAAAACUCA | |
| 1564324 | 1564339 | AACCAAUUAAAA | 155 | 2934 | 2956 | 1564333 | CCUUUUAUAUU | 467 |
| | | UAUAAAAGGCA | | | | | UUAAUUGGUU | |
| 1564329 | 1564341 | GCAACAUUAAGC | 156 | 2914 | 2936 | 1564334 | UUUUAAAAUGC | 468 |
| | | AUUUUAAAAUU | | | | | UUAAUGUUGC | |
| 1564326 | 1564337 | ACAUUCAUUUUA | 157 | 2987 | 3009 | 1564330 | UCACUAUAUUA | 469 |
| | | AUAUAGUGAUU | | | | | AAAUGAAUGU | |
| 1564327 | 1564338 | UAUAGUGAUUCU | 158 | 2974 | 2996 | 1564331 | AUGCAAAUCAG | 470 |
| | | GAUUUGCAUUA | | | | | AAUCACUAUA | |
| 1576614 | 1576627 | AUAGAGUAUUGU | 159 | 84 | 106 | 1576620 | UCAUACAACAC | 471 |
| | | GUUGUAUGAAG | | | | | AAUACUCUAU | |
| 1576613 | 1576624 | GCAGCUGUGAUC | 160 | 104 | 126 | 1576618 | UACUGUGAUGA | 472 |
| | | AUCACAGUAUA | | | | | UCACAGCUGC | |
| 1576616 | 1576629 | AAGUCUUACGGG | 161 | 64 | 86 | 1576622 | AGCUAAACACC | 473 |
| | | UGUUUAGCUGG | | | | | CGUAAGACUU | |
| 1576617 | 1576628 | AGCCAAAUAUGA | 162 | 144 | 166 | 1576623 | ACAGUUAUCUC | 474 |
| | | GAUAACUGUCU | | | | | AUAUUUGGCU | |
| 1576615 | 1576626 | UCUUCUUUUAGG | 163 | 124 | 146 | 1576619 | CCAAGGCUACC | 475 |
| | | UAGCCUUGGCA | | | | | UAAAAGAAGA | |
| 1576612 | 1576625 | UGGGGAGUUUUC | 164 | 44 | 66 | 1576621 | ACAGAGUCAGA | 476 |
| | | UGACUCUGUCA | | | | | AAACUCCCCA | |
| 1576632 | 1576643 | AUUUCAAUGGUU | 165 | 264 | 286 | 1576640 | AAGAGCCUCAA | 477 |
| | | GAGGCUCUUCU | | | | | CCAUUGAAAU | |
| 1576634 | 1576645 | CUGAAGUUUUAA | 166 | 184 | 206 | 1576639 | CUCGACCACUU | 478 |
| | | GUGGUCGAGAG | | | | | AAAACUUCAG | |
| 1576635 | 1576646 | AUACCAGCAGGA | 167 | 204 | 226 | 1576637 | GACUUCCUGUC | 479 |
| | | CAGGAAGUCUG | | | | | CUGCUGGUAU | |
| 1576630 | 1576647 | GAGAAAGAUAAA | 168 | 164 | 186 | 1576636 | UGCCAGCUUUU | 480 |
| | | AAGCUGGCAGC | | | | | UAUCUUUCUC | |
| 1576633 | 1576644 | GGUAUUGGACUU | 169 | 224 | 246 | 1576638 | UCAUGGAGAAA | 481 |
| | | UCUCCAUGAUA | | | | | GUCCAAUACC | |
| 1576631 | 1576642 | UCUUAUAGCUGA | 170 | 244 | 266 | 1576641 | CUCACUCGCUC | 482 |
| | | GCGAGUGAGGU | | | | | AGCUAUAAGA | |
| 1576652 | 1576664 | AUUAAGAUGAGA | 171 | 324 | 346 | 1576659 | CAAUUUCUGUC | 483 |
| | | CAGAAAUUGAU | | | | | UCAUCUUAAU | |
| 1576651 | 1576663 | UACAGAUCAGCA | 172 | 344 | 366 | 1576654 | UAUGUCUCUUG | 484 |
| | | AGAGACAUAUU | | | | | CUGAUCUGUA | |
| 1576648 | 1576661 | UUUGACGUGCUU | 173 | 284 | 306 | 1576657 | UGCCUCAACAA | 485 |
| | | GUUGAGGCAUU | | | | | GCACGUCAAA | |
| 1576649 | 1576660 | CUAGAGGUUGUA | 174 | 384 | 406 | 1576656 | AAGUUCUGCUA | 486 |
| | | GCAGAACUUCA | | | | | CAACCUCUAG | |
| 1576650 | 1576662 | UCAGAGAAGCAU | 175 | 364 | 386 | 1576655 | AUCAUCGUGAU | 487 |
| | | CACGAUGAUAC | | | | | GCUUCUCUGA | |
| 1576653 | 1576665 | GAUAAAUAGAUU | 176 | 304 | 326 | 1576658 | AAGCUACAGAA | 488 |
| | | CUGUAGCUUUU | | | | | UCUAUUUAUC | |
| 1576667 | 1576678 | ACAAAGAAACUC | 177 | 484 | 506 | 1576674 | UGAGUAGAAGA | 489 |
| | | UUCUACUCAGG | | | | | GUUUCUUUGU | |
| 1576666 | 1576677 | UGAUGUGGCAAG | 178 | 404 | 426 | 1576673 | GAUCUGCAGCU | 490 |
| | | CUGCAGAUCUA | | | | | UGCCACAUCA | |
| 1576671 | 1576683 | GUCUUAAUCUUG | 179 | 504 | 526 | 1576679 | UGAAAAGGUCA | 491 |
| | | ACCUUUUCACA | | | | | AGAUUAAGAC | |
| 1576669 | 1576681 | AUAUUGUUUUCC | 180 | 444 | 466 | 1576676 | AUGCAGACAGG | 492 |
| | | UGUCUGCAUGG | | | | | AAAACAAUAU | |
| 1576668 | 1576680 | AGGAAGUGGUCU | 181 | 464 | 486 | 1576672 | UUGUAUAACAG | 493 |
| | | GUUAUACAAUA | | | | | ACCACUUCCU | |
| 1576670 | 1576682 | UGGGAUGACAGA | 182 | 424 | 446 | 1576675 | AGCUUAAAAUC | 494 |
| | | UUUUAAGCUGA | | | | | UGUCAUCCCA | |
| 1576684 | 1576697 | UAUGGUAACAAU | 183 | 524 | 546 | 1576693 | CUAAAACUUAU | 495 |
| | | AAGUUUUAGUC | | | | | UGUUACCAUA | |
| 1576686 | 1576694 | GCAUUUUAUACA | 184 | 604 | 626 | 1576692 | CAAAAUAAGUG | 496 |
| | | CUUAUUUUGAA | | | | | UAUAAAAUGC | |
| 1576689 | 1576701 | AGAUCCAACAGA | 185 | 544 | 566 | 1576696 | AUGUAUUCAUC | 497 |
| | | UGAAUACAUAU | | | | | UGUUGGAUCU | |
| 1576688 | 1576700 | AAGCCCUUUUCA | 186 | 564 | 586 | 1576695 | UUGUAAACAUG | 498 |
| | | UGUUUACAAGA | | | | | AAAAGGGCUU | |
| 1576687 | 1576699 | GAAGUUAAUUUU | 187 | 584 | 606 | 1576690 | UUAUUUUCAAA | 499 |
| | | UGAAAAUAAAG | | | | | AAUUAACUUC | |
| 1576685 | 1576698 | UGUUGAGGAAAU | 188 | 624 | 646 | 1576691 | CAACUGUUGAU | 500 |
| | | CAACAGUUGCA | | | | | UUCCUCAACA | |
| 1576705 | 1576716 | AAGUGAACUUGU | 189 | 704 | 726 | 1576708 | GCACUGCCAAC | 501 |
| | | UGGCAGUGCAG | | | | | AAGUUCACUU | |
| 1576706 | 1576719 | CAGUUUUAAAAC | 190 | 684 | 706 | 1576713 | AAGAGUUUAGU | 502 |
| | | UAAACUCUUCA | | | | | UUUAAAACUG | |
| 1576704 | 1576714 | AAAAUAAUGCUU | 191 | 724 | 746 | 1576709 | UCAUAUAUAAA | 503 |
| | | UAUAUAUGAAG | | | | | GCAUUAUUUU | |
| 1576703 | 1576717 | AUAUUCACCUCA | 192 | 744 | 766 | 1576711 | UUACUCUUUUG | 504 |
| | | AAAGAGUAAAA | | | | | AGGUGAAUAU | |
| 1576702 | 1576715 | GGAUAGAAAUUU | 193 | 644 | 666 | 1576710 | AUGGCUCACAA | 505 |
| | | GUGAGCCAUGU | | | | | AUUUCUAUCC | |
| 1576707 | 1576718 | UCAUCUUCAGAA | 194 | 664 | 686 | 1576712 | CCAAAUCUUUU | 506 |
| | | AAGAUUUGGGA | | | | | CUGAAGAUGA | |
| 1576725 | 1576737 | UACUUAGUAUUA | 195 | 784 | 806 | 1576730 | AAGCUUCUUUA | 507 |
| | | AAGAAGCUUUU | | | | | AUACUAAGUA | |
| 1576723 | 1576734 | UUUUGAAUAAUG | 196 | 844 | 866 | 1576726 | AUGAAGUGUCA | 508 |
| | | ACACUUCAUUU | | | | | UUAUUCAAAA | |
| 1576722 | 1576733 | UGUGAGGAGUCA | 197 | 864 | 886 | 1576727 | AUAGUCCACUG | 509 |
| | | GUGGACUAUUU | | | | | ACUCCUCACA | |
| 1576724 | 1576736 | CUUGGUGAAGAC | 198 | 804 | 826 | 1576731 | AUUUUUCAGGU | 510 |
| | | CUGAAAAAUAC | | | | | CUUCACCAAG | |
| 1576721 | 1576732 | CUUUAUAAUAAG | 199 | 884 | 906 | 1576729 | AUCUGUUAUCU | 511 |
| | | AUAACAGAUGU | | | | | UAUUAUAAAG | |
| 1576720 | 1576735 | UUUACAUUGUAA | 200 | 764 | 786 | 1576728 | UAAUUUAUAUU | 512 |
| | | UAUAAAUUAUA | | | | | ACAAUGUAAA | |
| 1576742 | 1576754 | CAUAGAAAAAAU | 201 | 944 | 966 | 1576749 | AGAAAUUGUAU | 513 |
| | | ACAAUUUCUGU | | | | | UUUUUCUAUG | |
| 1576743 | 1576755 | UGUUUUAGAAUG | 202 | 924 | 946 | 1576748 | CAGAAUCUACA | 514 |
| | | UAGAUUCUGAU | | | | | UUCUAAAACA | |
| 1576741 | 1576751 | UUUAAAAGAUGU | 203 | 964 | 986 | 1576744 | GCCACAUUAAC | 515 |
| | | UAAUGUGGCAU | | | | | AUCUUUUAAA | |
| 1576740 | 1576750 | AGAGUAUGGCCU | 204 | 1004 | 1026 | 1576745 | GGAAAAGUAAG | 516 |
| | | UACUUUUCCAU | | | | | GCCAUACUCU | |
| 1576738 | 1576753 | GAUAGUUACUAC | 205 | 904 | 926 | 1576747 | GAACUAUUUGU | 517 |
| | | AAAUAGUUCUU | | | | | AGUAACUAUC | |
| 1576739 | 1576752 | AAAAGGAAUUUU | 206 | 1024 | 1046 | 1576746 | UUACAUAAUAA | 518 |
| | | AUUAUGUAAGA | | | | | AAUUCCUUUU | |
| 1576760 | 1576773 | AACAGAUUUAUG | 207 | 1084 | 1106 | 1576770 | UAGGUAAAUCA | 519 |
| | | AUUUACCUACA | | | | | UAAAUCUGUU | |
| 1576759 | 1576771 | UAUUAACCACCA | 208 | 1124 | 1146 | 1576763 | GAUGAGAACUG | 520 |
| | | GUUCUCAUCUG | | | | | GUGGUUAAUA | |
| 1576761 | 1576772 | AUUCUUUGAAAA | 209 | 1044 | 1066 | 1576766 | UAAGUAAUUUU | 521 |
| | | AAUUACUUAAA | | | | | UUCAAAGAAU | |
| 1576758 | 1576767 | CUGUUGAUCAUA | 210 | 1104 | 1126 | 1576762 | UCUAAGACAUA | 522 |
| | | UGUCUUAGAAC | | | | | UGAUCAACAG | |
| 1576757 | 1576769 | UGACUAAUCUCA | 211 | 1144 | 1166 | 1576765 | AUGUGACAGUG | 523 |
| | | CUGUCACAUAU | | | | | AGAUUAGUCA | |
| 1576756 | 1576768 | ACAUGUACUAGA | 212 | 1064 | 1086 | 1576764 | UCACAGAAUUC | 524 |
| | | AUUCUGUGAUU | | | | | UAGUACAUGU | |
| 1576777 | 1576786 | AAGUCUUAAGGU | 213 | 1264 | 1286 | 1576781 | AAUCAUGAAAC | 525 |
| | | UUCAUGAUUCC | | | | | CUUAAGACUU | |
| 1576778 | 1576791 | GAUAAUUCACUA | 214 | 1204 | 1226 | 1576785 | AAGGCACUGUA | 526 |
| | | CAGUGCCUUAA | | | | | GUGAAUUAUC | |
| 1576779 | 1576790 | UAAAUGAAGAUU | 215 | 1184 | 1206 | 1576784 | ACAGAAUCUAA | 527 |
| | | AGAUUCUGUUG | | | | | UCUUCAUUUA | |
| 1576776 | 1576787 | AGCUAGGUAACU | 216 | 1224 | 1246 | 1576780 | CUGAGCUAGAG | 528 |
| | | CUAGCUCAGAU | | | | | UUACCUAGCU | |
| 1576775 | 1576789 | UGCAAAAUCAUU | 217 | 1279 | 1301 | 1576782 | AGACUUCAGAA | 529 |
| | | CUGAAGUCUUA | | | | | UGAUUUUGCA | |
| 1576774 | 1576788 | UUGUUAGUAUAU | 218 | 1164 | 1186 | 1576783 | AUAUCACUAAU | 530 |
| | | UAGUGAUAUGA | | | | | AUACUAACAA | |
| 1576796 | 1576809 | UUUUCCUUGCCU | 219 | 1324 | 1346 | 1576806 | AUCCAAUGCAG | 531 |
| | | GCAUUGGAUGU | | | | | GCAAGGAAAA | |
| 1576793 | 1576803 | UUCAUAUAUUUU | 220 | 1384 | 1406 | 1576801 | UAUUUUUUAAA | 532 |
| | | UAAAAAAUACU | | | | | AAUAUAUGAA | |
| 1576797 | 1576808 | UGUUAGGCUGGA | 221 | 1304 | 1326 | 1576802 | GUCUUCCAUUC | 533 |
| | | AUGGAAGACAA | | | | | CAGCCUAACA | |
| 1576792 | 1576804 | CAACCUGCAAAA | 222 | 1284 | 1306 | 1576800 | UCAGAAUGAUU | 534 |
| | | UCAUUCUGAAG | | | | | UUGCAGGUUG | |
| 1576794 | 1576807 | ACUUGAGAUAAU | 223 | 1364 | 1386 | 1576799 | GAAAAAUAUAU | 535 |
| | | AUAUUUUUCUG | | | | | UAUCUCAAGU | |
| 1576795 | 1576805 | CUGUCACUGGAA | 224 | 1344 | 1366 | 1576798 | AUAAAAGAUUU | 536 |
| | | AUCUUUUAUUU | | | | | CCAGUGACAG | |
| 1576813 | 1576825 | UGGGCCAACAAG | 225 | 1444 | 1466 | 1576819 | UGAAAUGAACU | 537 |
| | | UUCAUUUCAAA | | | | | UGUUGGCCCA | |
| 1576810 | 1576821 | GUUAAUAUUUGG | 226 | 1404 | 1426 | 1576817 | AUUCUCUCUCC | 538 |
| | | AGAGAGAAUUC | | | | | AAAUAUUAAC | |
| 1576812 | 1576826 | CUAACCCCCAUG | 227 | 1484 | 1506 | 1576820 | ACCCUUGAACA | 539 |
| | | UUCAAGGGUCA | | | | | UGGGGGUUAG | |
| 1576814 | 1576824 | AAAAUAUAAUCU | 228 | 1424 | 1446 | 1576818 | CUAAUUAUUAG | 540 |
| | | AAUAAUUAGUU | | | | | AUUAUAUUUU | |
| 1576815 | 1576827 | GAAUUGUCAGCU | 229 | 1499 | 1521 | 1576822 | GGUUAGGGGAG | 541 |
| | | CCCCUAACCCC | | | | | CUGACAAUUC | |
| 1576811 | 1576823 | UCAGCUGUAGAU | 230 | 1464 | 1486 | 1576816 | AUCUAUUACAU | 542 |
| | | GUAAUAGAUGG | | | | | CUACAGCUGA | |
| 1576829 | 1576845 | UAAUACAUAUAA | 231 | 1604 | 1626 | 1576838 | UUUUGCGUGUU | 543 |
| | | CACGCAAAAUA | | | | | AUAUGUAUUA | |
| 1576830 | 1576840 | AUAUGUGUUAAU | 232 | 1584 | 1606 | 1576837 | AAACAGUAAAU | 544 |
| | | UUACUGUUUAU | | | | | UAACACAUAU | |
| 1576828 | 1576842 | UAUUGUAGGAAU | 233 | 1624 | 1646 | 1576839 | AUACACUAUAU | 545 |
| | | AUAGUGUAUAA | | | | | UCCUACAAUA | |
| 1576831 | 1576844 | UAUGUUAUCAGU | 234 | 1564 | 1586 | 1576834 | AUAAACCUUAC | 546 |
| | | AAGGUUUAUGG | | | | | UGAUAACAUA | |
| 1576833 | 1576841 | CCCACGAAUUGU | 235 | 1504 | 1526 | 1576836 | GGGGAGCUGAC | 547 |
| | | CAGCUCCCCUA | | | | | AAUUCGUGGG | |
| 1576832 | 1576843 | UGGUCAAUAGUA | 236 | 1544 | 1566 | 1576835 | CUAAUAGCCUA | 548 |
| | | GGCUAUUAGGU | | | | | CUAUUGACCA | |
| 1576848 | 1576859 | AGAAUGAAGACU | 237 | 1724 | 1746 | 1576853 | CAACAUAAAAG | 549 |
| | | UUUAUGUUGAC | | | | | UCUUCAUUCU | |
| 1576850 | 1576863 | AUAGUAAAUAUA | 238 | 1684 | 1706 | 1576860 | AAGAGAAAAUA | 550 |
| | | UUUUCUCUUUC | | | | | UAUUUACUAU | |
| 1576851 | 1576862 | UUCUUAUGAUUU | 239 | 1664 | 1686 | 1576856 | UUAUUUAGAAA | 551 |
| | | UCUAAAUAACA | | | | | AUCAUAAGAA | |
| 1576849 | 1576857 | GACCCACUUCCA | 240 | 1704 | 1726 | 1576852 | UUCAUUAAAUG | 552 |
| | | UUUAAUGAAUA | | | | | GAAGUGGGUC | |
| 1576847 | 1576858 | UACUCAAUGUGA | 241 | 1744 | 1766 | 1576854 | UCAUUGUCUUC | 553 |
| | | AGACAAUGAGA | | | | | ACAUUGAGUA | |
| 1576846 | 1576861 | ACAUUUUCUCUA | 242 | 1644 | 1666 | 1576855 | AAAGUAAGCUA | 554 |
| | | GCUUACUUUAU | | | | | GAGAAAAUGU | |
| 1576869 | 1576881 | GGGCUUGAAUUG | 243 | 1864 | 1886 | 1576876 | GGACCCUUGCA | 555 |
| | | CAAGGGUCCAC | | | | | AUUCAAGCCC | |
| 1576868 | 1576879 | CAUCUUUCUCCU | 244 | 1764 | 1786 | 1576873 | AGGCAGAGGAG | 556 |
| | | CCUCUGCCUAC | | | | | GAGAAAGAUG | |
| 1576864 | 1576877 | CUAAGACAAGAC | 245 | 1804 | 1826 | 1576871 | GGUCUUGCAGU | 557 |
| | | UGCAAGACCAA | | | | | CUUGUCUUAG | |
| 1576865 | 1576875 | UUCCCCCACUCC | 246 | 1824 | 1846 | 1576872 | GGGGUGUGGGG | 558 |
| | | CCACACCCCUA | | | | | AGUGGGGGAA | |
| 1576866 | 1576874 | CACUUAUACAUG | 247 | 1844 | 1866 | 1576870 | AAGAAUAUUCA | 559 |
| | | AAUAUUCUUUC | | | | | UGUAUAAGUG | |
| 1576867 | 1576880 | CAACGCCUUCUC | 248 | 1784 | 1806 | 1576878 | GGGGAGGAAGA | 560 |
| | | UUCCUCCCCAU | | | | | GAAGGCGUUG | |
| 1576882 | 1576895 | AGUUGACCCUUG | 249 | 1884 | 1906 | 1576889 | CUUGUUGUUCA | 561 |
| | | AACAACAAGGG | | | | | AGGGUCAACU | |
| 1576884 | 1576894 | UUUAGCUCAGUA | 250 | 1964 | 1986 | 1576888 | AGUCCACUCUA | 562 |
| | | GAGUGGACUGC | | | | | CUGAGCUAAA | |
| 1576883 | 1576896 | UAGCAUAGCUGG | 251 | 1984 | 2006 | 1576890 | AUUAUAGAUCC | 563 |
| | | AUCUAUAAUUU | | | | | AGCUAUGCUA | |
| 1576887 | 1576897 | AAAUAGCUAUAU | 252 | 1904 | 1926 | 1576891 | UGUAAUAGGAU | 564 |
| | | CCUAUUACAGU | | | | | AUAGCUAUUU | |
| 1576885 | 1576899 | UUUGGUUGAUAG | 253 | 1924 | 1946 | 1576892 | UUUCUUCCUCU | 565 |
| | | AGGAAGAAAAA | | | | | AUCAACCAAA | |
| 1576886 | 1576898 | UGCAAAAUAGAU | 254 | 1944 | 1966 | 1576893 | AUGGUAAGCAU | 566 |
| | | GCUUACCAUUU | | | | | CUAUUUUGCA | |
| 1576901 | 1576912 | CACUCAAUUUUA | 255 | 2084 | 2106 | 1576909 | CAACUGAAGUA | 567 |
| | | CUUCAGUUGUU | | | | | AAAUUGAGUG | |
| 1576903 | 1576913 | AUGUUCUGAAAU | 256 | 2044 | 2066 | 1576907 | CCUCUGACCAU | 568 |
| | | GGUCAGAGGAG | | | | | UUCAGAACAU | |
| 1576902 | 1576911 | GUUUUAUGAGUU | 257 | 2064 | 2086 | 1576906 | UCUUCCAAUAA | 569 |
| | | AUUGGAAGAUG | | | | | CUCAUAAAAC | |
| 1576900 | 1576915 | AUCAAGAAAAUA | 258 | 2004 | 2026 | 1576908 | AUUUAUAAUUA | 570 |
| | | AUUAUAAAUAG | | | | | UUUUCUUGAU | |
| 1576905 | 1576916 | AUUGAAAAUUUA | 259 | 2019 | 2041 | 1576910 | CUUGAUGAAUA | 571 |
| | | UUCAUCAAGAA | | | | | AAUUUUCAAU | |
| 1576904 | 1576917 | GAGAAAUUGAAA | 260 | 2024 | 2046 | 1576914 | UGAAUAAAUUU | 572 |
| | | AUUUAUUCAUC | | | | | UCAAUUUCUC | |
| 1576919 | 1576933 | ACAUGUCUUCAA | 261 | 2204 | 2226 | 1576926 | UAAUUUCAGUU | 573 |
| | | CUGAAAUUAAA | | | | | GAAGACAUGU | |
| 1576920 | 1576929 | AAAAUUGUUGGA | 262 | 2184 | 2206 | 1576925 | ACUGCAUAAUC | 574 |
| | | UUAUGCAGUAU | | | | | CAACAAUUUU | |
| 1576923 | 1576934 | UCAACUUAAAUU | 263 | 2124 | 2146 | 1576928 | AACUUUGGUAA | 575 |
| | | ACCAAAGUUUG | | | | | UUUAAGUUGA | |
| 1576922 | 1576935 | GACUUAAUUUUA | 264 | 2144 | 2166 | 1576931 | ACUAAAGUUUA | 576 |
| | | AACUUUAGUCA | | | | | AAAUUAAGUC | |
| 1576921 | 1576930 | UAUAGGUGUAAA | 265 | 2164 | 2186 | 1576924 | CUAAAAUAGUU | 577 |
| | | CUAUUUUAGAC | | | | | UACACCUAUA | |
| 1576918 | 1576932 | UUGGUGAAUAUA | 266 | 2104 | 2126 | 1576927 | GCUGGAAAAUA | 578 |
| | | UUUUCCAGCAC | | | | | UAUUCACCAA | |
| 1576937 | 1576950 | CAAGGUCAAUAU | 267 | 2304 | 2326 | 1576945 | GUAACAUCAAU | 579 |
| | | UGAUGUUACAU | | | | | AUUGACCUUG | |
| 1576938 | 1576947 | CAUAUUCUUUUA | 268 | 2284 | 2306 | 1576943 | CAUGGUUACUA | 580 |
| | | GUAACCAUGUU | | | | | AAAGAAUAUG | |
| 1576939 | 1576949 | GUUUUAGAAGAU | 269 | 2264 | 2286 | 1576942 | AUUAACCAUAU | 581 |
| | | AUGGUUAAUUA | | | | | CUUCUAAAAC | |
| 1576936 | 1576951 | AUAGUUAUAUUA | 270 | 2219 | 2241 | 1576944 | ACAUGUUACUA | 582 |
| | | GUAACAUGUCU | | | | | AUAUAACUAU | |
| 1576941 | 1576952 | UUACACAUCCUC | 271 | 2244 | 2266 | 1576946 | AAAAGAGUAGA | 583 |
| | | UACUCUUUUAA | | | | | GGAUGUGUAA | |
| 1576940 | 1576953 | UAAUAAUAGUUA | 272 | 2224 | 2246 | 1576948 | UUACUAAUAUA | 584 |
| | | UAUUAGUAACA | | | | | ACUAUUAUUA | |
| 1576956 | 1576966 | AUCUCUAGUAUC | 273 | 2384 | 2406 | 1576961 | CCCAUGAGAGA | 585 |
| | | UCUCAUGGGUU | | | | | UACUAGAGAU | |
| 1576957 | 1576967 | UAGUAUCUCUCA | 274 | 2379 | 2401 | 1576960 | CUGAACCCAUG | 586 |
| | | UGGGUUCAGAA | | | | | AGAGAUACUA | |
| 1576959 | 1576970 | UCUCCUUCACUA | 275 | 2344 | 2366 | 1576964 | CUAACUCAAUA | 587 |
| | | UUGAGUUAGCA | | | | | GUGAAGGAGA | |
| 1576955 | 1576968 | CCAAACACUUUC | 276 | 2404 | 2426 | 1576963 | UGGGGAGUGGA | 588 |
| | | CACUCCCCAUC | | | | | AAGUGUUUGG | |
| 1576958 | 1576971 | GUUCAGAAAAUU | 277 | 2364 | 2386 | 1576965 | ACACUAUUAAA | 589 |
| | | UAAUAGUGUCU | | | | | UUUUCUGAAC | |
| 1576954 | 1576969 | GCAACACUUGUG | 278 | 2324 | 2346 | 1576962 | GGUUUCUUACA | 590 |
| | | UAAGAAACCAA | | | | | CAAGUGUUGC | |
| 1576974 | 1576985 | UUGUUUUUAGGA | 279 | 2484 | 2506 | 1576979 | GCUGGAAAUUC | 591 |
| | | AUUUCCAGCUU | | | | | CUAAAAACAA | |
| 1576976 | 1576988 | CUUGUAGAUGAG | 280 | 2464 | 2486 | 1576984 | AAGUGACGCCU | 592 |
| | | GCGUCACUUAA | | | | | CAUCUACAAG | |
| 1576977 | 1576989 | UAAGAAAUCUCU | 281 | 2444 | 2466 | 1576982 | CAGAAGGGCAG | 593 |
| | | GCCCUUCUGUU | | | | | AGAUUUCUUA | |
| 1576975 | 1576983 | UUUAGGAAUUUC | 282 | 2479 | 2501 | 1576978 | UACAAGCUGGA | 594 |
| | | CAGCUUGUAGA | | | | | AAUUCCUAAA | |
| 1576972 | 1576986 | GUUCUUCAGAUA | 283 | 2424 | 2446 | 1576981 | GUUCAGGGAUA | 595 |
| | | UCCCUGAACCA | | | | | UCUGAAGAAC | |
| 1576973 | 1576987 | GUUGUUUAUAAG | 284 | 2504 | 2526 | 1576980 | AGUAGAAAGCU | 596 |
| | | CUUUCUACUUG | | | | | UAUAAACAAC | |
| 1576993 | 1577004 | GAAUAUGACUAA | 285 | 2604 | 2626 | 1576996 | UUGAACUGAUU | 597 |
| | | UCAGUUCAAAU | | | | | AGUCAUAUUC | |
| 1576995 | 1577006 | GACUAGGGAUAC | 286 | 2564 | 2586 | 1577000 | AUACAGAAAGU | 598 |
| | | UUUCUGUAUUG | | | | | AUCCCUAGUC | |
| 1576992 | 1577002 | ACAGUGUAAUCA | 287 | 2619 | 2641 | 1576998 | AUAUUCCUUUG | 599 |
| | | AAGGAAUAUGA | | | | | AUUACACUGU | |
| 1576994 | 1577007 | UUGGUAAUUUAC | 288 | 2544 | 2566 | 1577001 | CUGGUUUUAGU | 600 |
| | | UAAAACCAGUG | | | | | AAAUUACCAA | |
| 1576990 | 1577005 | GUGAGGUGAGUG | 289 | 2524 | 2546 | 1576999 | CAGGUGAUACA | 601 |
| | | UAUCACCUGUU | | | | | CUCACCUCAC | |
| 1576991 | 1577003 | AAUUUUCCACUU | 290 | 2584 | 2606 | 1576997 | CUUAAAAACAA | 602 |
| | | GUUUUUAAGAC | | | | | GUGGAAAAUU | |
| 1577013 | 1577024 | UUUACUGAGAAA | 291 | 2644 | 2666 | 1577018 | UACAAUAUUUU | 603 |
| | | AAUAUUGUAAC | | | | | UCUCAGUAAA | |
| 1577008 | 1577022 | AACAAACAGUGU | 292 | 2624 | 2646 | 1577016 | CCUUUGAUUAC | 604 |
| | | AAUCAAAGGAA | | | | | ACUGUUUGUU | |
| 1577011 | 1577021 | UCUAUCAAAGAA | 293 | 2684 | 2706 | 1577014 | UGUUCUUCAUU | 605 |
| | | UGAAGAACAAA | | | | | CUUUGAUAGA | |
| 1577010 | 1577020 | AAUAAGAUUUUA | 294 | 2699 | 2721 | 1577015 | GAUAGAAAUUA | 606 |
| | | AUUUCUAUCAA | | | | | AAAUCUUAUU | |
| 1577012 | 1577025 | AAAAAAAUUAGU | 295 | 2664 | 2686 | 1577019 | ACAGAAAUAAC | 607 |
| | | UAUUUCUGUUU | | | | | UAAUUUUUUU | |
| 1577009 | 1577023 | CACAGAAUAAGA | 296 | 2704 | 2726 | 1577017 | AAAUUAAAAUC | 608 |
| | | UUUUAAUUUCU | | | | | UUAUUCUGUG | |
| 1577028 | 1577041 | UUAUUUAAAGAG | 297 | 2759 | 2781 | 1577038 | UAGAAUAAACU | 609 |
| | | UUUAUUCUACU | | | | | CUUUAAAUAA | |
| 1577026 | 1577037 | UUAUAGUAUUCU | 298 | 2724 | 2746 | 1577032 | GAGGAUUACAG | 610 |
| | | GUAAUCCUCAC | | | | | AAUACUAUAA | |
| 1577030 | 1577043 | UUGUUUUCUUAU | 299 | 2804 | 2826 | 1577039 | GAAUAAGAUAU | 611 |
| | | AUCUUAUUCUU | | | | | AAGAAAACAA | |
| 1577027 | 1577035 | AAUAAUUAUUUA | 300 | 2764 | 2786 | 1577033 | UAAACUCUUUA | 612 |
| | | AAGAGUUUAUU | | | | | AAUAAUUAUU | |
| 1577031 | 1577042 | AUUCUACUUUAU | 301 | 2744 | 2766 | 1577036 | ACUCAAAUUAU | 613 |
| | | AAUUUGAGUUA | | | | | AAAGUAGAAU | |
| 1577029 | 1577040 | CUUUACACUUUA | 302 | 2784 | 2806 | 1577034 | UCUUCAUCAUA | 614 |
| | | UGAUGAAGAAU | | | | | AAGUGUAAAG | |
| 1577045 | 1577058 | GCAUUUUAAAAU | 303 | 2904 | 2926 | 1577052 | CUAAAAACAAU | 615 |
| | | UGUUUUUAGUG | | | | | UUUAAAAUGC | |
| 1577049 | 1577060 | AGAACUUAUUCU | 304 | 2864 | 2886 | 1577054 | UUCUACUAUAG | 616 |
| | | AUAGUAGAACA | | | | | AAUAAGUUCU | |
| 1577046 | 1577057 | CUGUAAAUUAAG | 305 | 2879 | 2901 | 1577050 | AGUUCUUAUCU | 617 |
| | | AUAAGAACUUA | | | | | UAAUUUACAG | |
| 1577047 | 1577056 | GUGCCCUGUAAA | 306 | 2884 | 2906 | 1577051 | UUAUCUUAAUU | 618 |
| | | UUAAGAUAAGA | | | | | UACAGGGCAC | |
| 1577048 | 1577061 | ACAUAUUUGAGC | 307 | 2844 | 2866 | 1577055 | AUACUAAAUGC | 619 |
| | | AUUUAGUAUAU | | | | | UCAAAUAUGU | |
| 1577044 | 1577059 | UAUUAAAUUUUA | 308 | 2824 | 2846 | 1577053 | AUUUAUUUUUA | 620 |
| | | AAAAUAAAUUG | | | | | AAAUUUAAUA | |
| 1577066 | 1577075 | AAUAUAAAAGGC | 309 | 2924 | 2946 | 1577071 | CUUAAUGUUGC | 621 |
| | | AACAUUAAGCA | | | | | CUUUUAUAUU | |
| 1577067 | 1577079 | UUCAAGAACAUU | 310 | 2994 | 3016 | 1577073 | AUUAAAAUGAA | 622 |
| | | CAUUUUAAUAU | | | | | UGUUCUUGAA | |
| 1577065 | 1577078 | AUAUAUUCUUAA | 311 | 2944 | 2966 | 1577072 | UUUAAUUGGUU | 623 |
| | | CCAAUUAAAAU | | | | | AAGAAUAUAU | |
| 1577064 | 1577077 | CUGAUUUGCAUU | 312 | 2964 | 2986 | 1577069 | UAUUUGUUUAA | 624 |
| | | AAACAAAUAUA | | | | | UGCAAAUCAG | |
| 1577063 | 1577076 | AUUUUAAUAUAG | 313 | 2981 | 3003 | 1577068 | UCAGAAUCACU | 625 |
| | | UGAUUCUGAUU | | | | | AUAUUAAAAU | |
| 1577062 | 1577074 | AAAAGGCAACAU | 314 | 2919 | 2941 | 1577070 | AAAUGCUUAAU | 626 |
| | | UAAGCAUUUUA | | | | | GUUGCCUUUU | |

Oligomeric duplexes described above were shown to reduce PLN RNA in single dose as well as in a dose dependent effect in cultured cells. We have found modification of oligomeric duplex compounds directed to PLN, as well as conjugated compounds comprising an oligomeric duplex conjugated to a cell targeting moiety, e.g., a transferin receptor ligand (e.g., a bicycle peptide compound targeting TfrRI) improves activity and/or other properties, such as pharmacokinetic and/or pharinacodynamic (e.g., duration of action, specificity of action, tolerability) of PLN-targeted compounds. Modifications provided herein may be applied to any one of the oligomeric compounds previously described. For example, improved modifications to oligomeric antisense and sense oligonucleotides, including specific modified sugar, nucleobase and/or internucleoside linkages, or motifs comprising any one or more of such modifications, may be incorporated into the nucleobase sequences previously described. Further, conjugation to cell targeting moieties as provided herein may be applied to previously described antisense and/or sense oligonucleotide nucleobase sequences to confer improved compounds.

Compound Nos. 1343077 (Table A1) and 1436542 (Table A2) are comparator compounds previously disclosed in International publication no. WO2022173976. Compound 1436542 is conjugated to a 6-palmitamidohexyl phosphoryl conjugate group attached to the 5′-OH of the oligonucleotide. The structure for the conjugate group 5′-C16 is depicted in Example 1.

TABLE A1
Modified oligonucleotide comparator targeted to human PLN

| | SEQ | SEQ ID | | | | |
|---|---|---|---|---|---|---|
| | ID No: | No: 2 | | | Internucleoside | SEQ |
| Compound | 2 Start | Stop | Sequence | Sugar Motif | Linkage | ID |
| No. | Site | Site | (5′ to 3′) | (5′ to 3′) | (5′ to 3′) | NO |
| 1343077 | 5501 | 5516 | ACACGAGTATATTAGG | kkkddddddddddkkk | sssssssssssssss | 1024 |

TABLE A2
5′ 6-palmitamidohexyl conjugated modified oligonucleotide comparator targeted to human
PLN

| | SEQ | SEQ | | | | |
|---|---|---|---|---|---|---|
| | ID No: | ID No: | | | Internucleoside | SEQ |
| Compound | 2 Start | 2 Stop | Sequence | Sugar Motif | Linkage | ID |
| No. | Site | Site | (5′ to 3′) | (5′ to 3′) | (5′ to 3′) | NO |
| 1436542 | 5501 | 5516 | ACACGAGTATATTAGG | kkkddddddddddkkk | sssssssssssssss | 1025 |

### Example B: Oligomeric Compounds that Target Human PLN

Design of Antisense Oligonucleotides that Target Human PIN

Oligomeric compounds comprising antisense oligonucleotides complementary to a human PLN nucleic acid, and sense oligonucleotides complementary to the antisense oligonucleotides were designed having nucleobase sequences as shown in Table B1 (including Table B1a and Table B1b) and Table B2, respectively. Each antisense oligonucleotide in Table B1 is 23 nucleosides in length having the nucleobase sequence in Table B1; has a sugar motif (from 5′ to 3′) of: yfyyyfyyyyyyyfyfyyyyyyy; wherein ‘y’ represents a 2′-OMe sugar moiety and ‘f’ represents a 2′-fluoro sugar moiety; and an intemnucleoside linkage motif (from 5′ to 3′) of: ssooooooooooooooooooss; wherein ‘o’ represents a phosphodiester intemnucleoside linkage and ‘s’ represents a phosphorothioate intemnucleoside linkage. Each cytosine residue is a non-methylated cytosine. Each antisense oligonucleotide has a terminal phosphate at the 5′-end. “Staff site” indicates the 5′-most nucleoside to which the antisense oligonucleotide is complementary in the target nucleic acid sequence. “Stop site” indicates the 3′-most nucleoside to which the antisense oligonucleotide is complementary in the target nucleic acid sequence. Each antisense oligonucleotide is 10000 complementary to SEQ ID NO: 1 (GenBank Accession NM_002667.4), to SEQ ID NO: 2 (GenBank Accession NC_000006.12 truncated from nucleosides 118545001 to 118565000), or to both, with the exception of a single mismatch at the 5′ end for certain sequences.

TABLE B1
Antisense oligonucleotides (antisense oligo.) targeted to human PLN

| | SEQ ID | SEQ ID | SEQ ID | SEQ ID | | |
|---|---|---|---|---|---|---|
| Antisense | NO: 1 | NO: 1 | NO: 2 | NO: 2 | | |
| Oligo. | Antisense | Antisense | Antisense | Antisense | | SEQ |
| Compound | Oligo. | Oligo. | Oligo. | Oligo. | | ID |
| No. | Start Site | Stop Site | Start Site | Stop Site | Antisense Strand Sequence (5′ to 3′) | NO |
| 1716582 | 298 | 320 | 13992 | 14014 | UAGAUUCUGUAGCUUUUGACGUG | 627 |
| 1716594 | 302 | 324 | 13996 | 14018 | UAAAUAGAUUCUGUAGCUUUUGA | 628 |
| 1716603 | 305 | 327 | 13999 | 14021 | UGAUAAAUAGAUUCUGUAGCUUU | 629 |
| 1716606 | 306 | 328 | 14000 | 14022 | UUGAUAAAUAGAUUCUGUAGCUU | 630 |
| 1716639 | 317 | 339 | 14011 | 14033 | UGAGACAGAAAUUGAUAAAUAGA | 631 |
| 1716654 | 322 | 344 | 14016 | 14038 | UAAGAUGAGACAGAAAUUGAUAA | 632 |
| 1716657 | 323 | 345 | 14017 | 14039 | UUAAGAUGAGACAGAAAUUGAUA | 633 |
| 1716663 | 325 | 347 | 14019 | 14041 | UAUUAAGAUGAGACAGAAAUUGA | 634 |
| 1716696 | 506 | 528 | 14200 | 14222 | UAGUCUUAAUCUUGACCUUUUCA | 635 |
| 1716699 | 507 | 529 | 14201 | 14223 | UUAGUCUUAAUCUUGACCUUUUC | 636 |
| 1716702 | 508 | 530 | 14202 | 14224 | UUUAGUCUUAAUCUUGACCUUUU | 637 |
| 1716705 | 509 | 531 | 14203 | 14225 | UUUUAGUCUUAAUCUUGACCUUU | 638 |
| 1716717 | 513 | 535 | 14207 | 14229 | UAAGUUUUAGUCUUAAUCUUGAC | 639 |
| 1716735 | 519 | 541 | 14213 | 14235 | UAACAAUAAGUUUUAGUCUUAAU | 640 |
| 1716744 | 522 | 544 | 14216 | 14238 | UGGUAACAAUAAGUUUUAGUCUU | 641 |
| 1716750 | 524 | 546 | 14218 | 14240 | UAUGGUAACAAUAAGUUUUAGUC | 642 |
| 1716771 | 541 | 563 | 14235 | 14257 | UCCAACAGAUGAAUACAUAUGGU | 643 |
| 1716795 | 548 | 570 | 14242 | 14264 | UACAAGAUCCAACAGAUGAAUAC | 644 |
| 1716798 | 549 | 571 | 14243 | 14265 | UUACAAGAUCCAACAGAUGAAUA | 645 |
| 1716801 | 550 | 572 | 14244 | 14266 | UUUACAAGAUCCAACAGAUGAAU | 646 |
| 1716807 | 552 | 574 | 14246 | 14268 | UGUUUACAAGAUCCAACAGAUGA | 647 |
| 1716816 | 555 | 577 | 14249 | 14271 | UCAUGUUUACAAGAUCCAACAGA | 648 |
| 1716819 | 556 | 578 | 14250 | 14272 | UUCAUGUUUACAAGAUCCAACAG | 649 |
| 1716822 | 557 | 579 | 14251 | 14273 | UUUCAUGUUUACAAGAUCCAACA | 650 |
| 1716825 | 558 | 580 | 14252 | 14274 | UUUUCAUGUUUACAAGAUCCAAC | 651 |
| 1716846 | 596 | 618 | 14290 | 14312 | UACACUUAUUUUGAAGUUAAUUU | 652 |
| 1716855 | 598 | 620 | 14292 | 14314 | UAUACACUUAUUUUGAAGUUAAU | 653 |
| 1716858 | 599 | 621 | 14293 | 14315 | UUAUACACUUAUUUUGAAGUUAA | 654 |
| 1716861 | 600 | 622 | 14294 | 14316 | UUUAUACACUUAUUUUGAAGUUA | 655 |
| 1716864 | 601 | 623 | 14295 | 14317 | UUUUAUACACUUAUUUUGAAGUU | 656 |
| 1716876 | 605 | 627 | 14299 | 14321 | UGCAUUUUAUACACUUAUUUUGA | 657 |
| 1716879 | 606 | 628 | 14300 | 14322 | UUGCAUUUUAUACACUUAUUUUG | 658 |
| 1716900 | 613 | 635 | 14307 | 14329 | UCAACAGUUGCAUUUUAUACACU | 659 |
| 1716903 | 665 | 687 | 14359 | 14381 | UUCAUCUUCAGAAAAGAUUUGGG | 660 |
| 1716909 | 667 | 689 | 14361 | 14383 | UCUUCAUCUUCAGAAAAGAUUUG | 661 |
| 1716927 | 672 | 694 | 14366 | 14388 | UAAACUCUUCAUCUUCAGAAAAG | 662 |
| 1716945 | 678 | 700 | 14372 | 14394 | UAAAACUAAACUCUUCAUCUUCA | 663 |
| 1716948 | 679 | 701 | 14373 | 14395 | UUAAAACUAAACUCUUCAUCUUC | 664 |
| 1716951 | 680 | 702 | 14374 | 14396 | UUUAAAACUAAACUCUUCAUCUU | 665 |
| 1716954 | 681 | 703 | 14375 | 14397 | UUUUAAAACUAAACUCUUCAUCU | 666 |
| 1716969 | 686 | 708 | 14380 | 14402 | UGCAGUUUUAAAACUAAACUCUU | 667 |
| 1716987 | 692 | 714 | 14386 | 14408 | UGGCAGUGCAGUUUUAAAACUAA | 668 |
| 1716990 | 693 | 715 | 14387 | 14409 | UUGGCAGUGCAGUUUUAAAACUA | 669 |
| 1716996 | 1676 | 1698 | 15370 | 15392 | UAUAUUUUCUCUUUCUUAUGAUU | 670 |
| 1717008 | 1680 | 1702 | 15374 | 15396 | UAAAUAUAUUUUCUCUUUCUUAU | 671 |
| 1717017 | 1683 | 1705 | 15377 | 15399 | UAGUAAAUAUAUUUUCUCUUUCU | 672 |
| 1717029 | 1687 | 1709 | 15381 | 15403 | UGAAUAGUAAAUAUAUUUUCUCU | 673 |
| 1717038 | 1690 | 1712 | 15384 | 15406 | UAAUGAAUAGUAAAUAUAUUUUC | 674 |
| 1717041 | 1691 | 1713 | 15385 | 15407 | UUAAUGAAUAGUAAAUAUAUUUU | 675 |
| 1717044 | 1692 | 1714 | 15386 | 15408 | UUUAAUGAAUAGUAAAUAUAUUU | 676 |
| 1716573 | 295 | 316 | 13989 | 14010 | UUUCUGUAGCUUUUGACGUGCUU | 677 |
| 1716576 | 296 | 317 | 13990 | 14011 | UAUUCUGUAGCUUUUGACGUGCU | 678 |
| 1716579 | 297 | 318 | 13991 | 14012 | UGAUUCUGUAGCUUUUGACGUGC | 679 |
| 1716585 | 299 | 320 | 13993 | 14014 | UUAGAUUCUGUAGCUUUUGACGU | 680 |
| 1716588 | 300 | 321 | 13994 | 14015 | UAUAGAUUCUGUAGCUUUUGACG | 681 |
| 1716591 | 301 | 322 | 13995 | 14016 | UAAUAGAUUCUGUAGCUUUUGAC | 682 |
| 1716597 | 303 | 324 | 13997 | 14018 | UUAAAUAGAUUCUGUAGCUUUUG | 683 |
| 1716600 | 304 | 325 | 13998 | 14019 | UAUAAAUAGAUUCUGUAGCUUUU | 684 |
| 1716609 | 307 | 328 | 14001 | 14022 | UUUGAUAAAUAGAUUCUGUAGCU | 685 |
| 1716612 | 308 | 329 | 14002 | 14023 | UAUUGAUAAAUAGAUUCUGUAGC | 686 |
| 1716615 | 309 | 330 | 14003 | 14024 | UAAUUGAUAAAUAGAUUCUGUAG | 687 |
| 1716618 | 310 | 331 | 14004 | 14025 | UAAAUUGAUAAAUAGAUUCUGUA | 688 |
| 1716621 | 311 | 332 | 14005 | 14026 | UGAAAUUGAUAAAUAGAUUCUGU | 689 |
| 1716624 | 312 | 333 | 14006 | 14027 | UAGAAAUUGAUAAAUAGAUUCUG | 690 |
| 1716627 | 313 | 334 | 14007 | 14028 | UCAGAAAUUGAUAAAUAGAUUCU | 691 |
| 1716630 | 314 | 335 | 14008 | 14029 | UACAGAAAUUGAUAAAUAGAUUC | 692 |
| 1716633 | 315 | 336 | 14009 | 14030 | UGACAGAAAUUGAUAAAUAGAUU | 693 |
| 1716636 | 316 | 337 | 14010 | 14031 | UAGACAGAAAUUGAUAAAUAGAU | 694 |
| 1716642 | 318 | 339 | 14012 | 14033 | UUGAGACAGAAAUUGAUAAAUAG | 695 |
| 1716645 | 319 | 340 | 14013 | 14034 | UAUGAGACAGAAAUUGAUAAAUA | 696 |
| 1716648 | 320 | 341 | 14014 | 14035 | UGAUGAGACAGAAAUUGAUAAAU | 697 |
| 1716651 | 321 | 342 | 14015 | 14036 | UAGAUGAGACAGAAAUUGAUAAA | 698 |
| 1716660 | 324 | 345 | 14018 | 14039 | UUUAAGAUGAGACAGAAAUUGAU | 699 |
| 1716666 | 326 | 347 | 14020 | 14041 | UUAUUAAGAUGAGACAGAAAUUG | 700 |
| 1716669 | 327 | 348 | 14021 | 14042 | UAUAUUAAGAUGAGACAGAAAUU | 701 |
| 1716672 | 328 | 349 | 14022 | 14043 | UCAUAUUAAGAUGAGACAGAAAU | 702 |
| 1716675 | 329 | 350 | 14023 | 14044 | UACAUAUUAAGAUGAGACAGAAA | 703 |
| 1716678 | 330 | 351 | 14024 | 14045 | UGACAUAUUAAGAUGAGACAGAA | 704 |
| 1716681 | 331 | 352 | 14025 | 14046 | UAGACAUAUUAAGAUGAGACAGA | 705 |
| 1716684 | 332 | 353 | 14026 | 14047 | UGAGACAUAUUAAGAUGAGACAG | 706 |
| 1716687 | 333 | 354 | 14027 | 14048 | UAGAGACAUAUUAAGAUGAGACA | 707 |
| 1716690 | 334 | 355 | 14028 | 14049 | UAAGAGACAUAUUAAGAUGAGAC | 708 |
| 1716693 | 505 | 526 | 14199 | 14220 | UGUCUUAAUCUUGACCUUUUCAC | 709 |
| 1716708 | 510 | 531 | 14204 | 14225 | UUUUUAGUCUUAAUCUUGACCUU | 710 |
| 1716711 | 511 | 532 | 14205 | 14226 | UGUUUUAGUCUUAAUCUUGACCU | 711 |
| 1716714 | 512 | 533 | 14206 | 14227 | UAGUUUUAGUCUUAAUCUUGACC | 712 |
| 1716720 | 514 | 535 | 14208 | 14229 | UUAAGUUUUAGUCUUAAUCUUGA | 713 |
| 1716723 | 515 | 536 | 14209 | 14230 | UAUAAGUUUUAGUCUUAAUCUUG | 714 |
| 1716726 | 516 | 537 | 14210 | 14231 | UAAUAAGUUUUAGUCUUAAUCUU | 715 |
| 1716729 | 517 | 538 | 14211 | 14232 | UCAAUAAGUUUUAGUCUUAAUCU | 716 |
| 1716732 | 518 | 539 | 14212 | 14233 | UACAAUAAGUUUUAGUCUUAAUC | 717 |
| 1716738 | 520 | 541 | 14214 | 14235 | UUAACAAUAAGUUUUAGUCUUAA | 718 |
| 1716741 | 521 | 542 | 14215 | 14236 | UGUAACAAUAAGUUUUAGUCUUA | 719 |
| 1716747 | 523 | 544 | 14217 | 14238 | UUGGUAACAAUAAGUUUUAGUCU | 720 |
| 1716753 | 535 | 556 | 14229 | 14250 | UGAUGAAUACAUAUGGUAACAAU | 721 |
| 1716756 | 536 | 557 | 14230 | 14251 | UAGAUGAAUACAUAUGGUAACAA | 722 |
| 1716759 | 537 | 558 | 14231 | 14252 | UCAGAUGAAUACAUAUGGUAACA | 723 |
| 1716762 | 538 | 559 | 14232 | 14253 | UACAGAUGAAUACAUAUGGUAAC | 724 |
| 1716765 | 539 | 560 | 14233 | 14254 | UAACAGAUGAAUACAUAUGGUAA | 725 |
| 1716768 | 540 | 561 | 14234 | 14255 | UCAACAGAUGAAUACAUAUGGUA | 726 |
| 1716774 | 542 | 563 | 14236 | 14257 | UUCCAACAGAUGAAUACAUAUGG | 727 |
| 1716777 | 543 | 564 | 14237 | 14258 | UAUCCAACAGAUGAAUACAUAUG | 728 |
| 1716780 | 544 | 565 | 14238 | 14259 | UGAUCCAACAGAUGAAUACAUAU | 729 |
| 1716783 | 545 | 566 | 14239 | 14260 | UAGAUCCAACAGAUGAAUACAUA | 730 |
| 1716786 | 546 | 567 | 14240 | 14261 | UAAGAUCCAACAGAUGAAUACAU | 731 |
| 1716792 | 547 | 568 | 14241 | 14262 | UCAAGAUCCAACAGAUGAAUACA | 732 |
| 1716804 | 551 | 572 | 14245 | 14266 | UUUUACAAGAUCCAACAGAUGAA | 733 |
| 1716810 | 553 | 574 | 14247 | 14268 | UUGUUUACAAGAUCCAACAGAUG | 734 |
| 1716813 | 554 | 575 | 14248 | 14269 | UAUGUUUACAAGAUCCAACAGAU | 735 |
| 1716828 | 559 | 580 | 14253 | 14274 | UUUUUCAUGUUUACAAGAUCCAA | 736 |
| 1716831 | 560 | 581 | 14254 | 14275 | UCUUUUCAUGUUUACAAGAUCCA | 737 |
| 1716834 | 561 | 582 | 14255 | 14276 | UCCUUUUCAUGUUUACAAGAUCC | 738 |
| 1716837 | 562 | 583 | 14256 | 14277 | UCCCUUUUCAUGUUUACAAGAUC | 739 |
| 1716840 | 563 | 584 | 14257 | 14278 | UGCCCUUUUCAUGUUUACAAGAU | 740 |
| 1716843 | 595 | 616 | 14289 | 14310 | UCACUUAUUUUGAAGUUAAUUUU | 741 |
| 1716849 | 597 | 618 | 14291 | 14312 | UUACACUUAUUUUGAAGUUAAUU | 742 |
| 1716867 | 602 | 623 | 14296 | 14317 | UUUUUAUACACUUAUUUUGAAGU | 743 |
| 1716870 | 603 | 624 | 14297 | 14318 | UAUUUUAUACACUUAUUUUGAAG | 744 |
| 1716873 | 604 | 625 | 14298 | 14319 | UCAUUUUAUACACUUAUUUUGAA | 745 |
| 1716882 | 607 | 628 | 14301 | 14322 | UUUGCAUUUUAUACACUUAUUUU | 746 |
| 1716885 | 608 | 629 | 14302 | 14323 | UGUUGCAUUUUAUACACUUAUUU | 747 |
| 1716888 | 609 | 630 | 14303 | 14324 | UAGUUGCAUUUUAUACACUUAUU | 748 |
| 1716891 | 610 | 631 | 14304 | 14325 | UCAGUUGCAUUUUAUACACUUAU | 749 |
| 1716894 | 611 | 632 | 14305 | 14326 | UACAGUUGCAUUUUAUACACUUA | 750 |
| 1716897 | 612 | 633 | 14306 | 14327 | UAACAGUUGCAUUUUAUACACUU | 751 |
| 1716906 | 666 | 687 | 14360 | 14381 | UUUCAUCUUCAGAAAAGAUUUGG | 752 |
| 1716915 | 668 | 689 | 14362 | 14383 | UUCUUCAUCUUCAGAAAAGAUUU | 753 |
| 1716918 | 669 | 690 | 14363 | 14384 | UCUCUUCAUCUUCAGAAAAGAUU | 754 |
| 1716921 | 670 | 691 | 14364 | 14385 | UACUCUUCAUCUUCAGAAAAGAU | 755 |
| 1716924 | 671 | 692 | 14365 | 14386 | UAACUCUUCAUCUUCAGAAAAGA | 756 |
| 1716930 | 673 | 694 | 14367 | 14388 | UUAAACUCUUCAUCUUCAGAAAA | 757 |
| 1716933 | 674 | 695 | 14368 | 14389 | UCUAAACUCUUCAUCUUCAGAAA | 758 |
| 1716936 | 675 | 696 | 14369 | 14390 | UACUAAACUCUUCAUCUUCAGAA | 759 |
| 1716939 | 676 | 697 | 14370 | 14391 | UAACUAAACUCUUCAUCUUCAGA | 760 |
| 1716942 | 677 | 698 | 14371 | 14392 | UAAACUAAACUCUUCAUCUUCAG | 761 |
| 1716957 | 682 | 703 | 14376 | 14397 | UUUUUAAAACUAAACUCUUCAUC | 762 |
| 1716960 | 683 | 704 | 14377 | 14398 | UGUUUUAAAACUAAACUCUUCAU | 763 |
| 1716963 | 684 | 705 | 14378 | 14399 | UAGUUUUAAAACUAAACUCUUCA | 764 |
| 1716966 | 685 | 706 | 14379 | 14400 | UCAGUUUUAAAACUAAACUCUUC | 765 |
| 1716972 | 687 | 708 | 14381 | 14402 | UUGCAGUUUUAAAACUAAACUCU | 766 |
| 1716975 | 688 | 709 | 14382 | 14403 | UGUGCAGUUUUAAAACUAAACUC | 767 |
| 1716978 | 689 | 710 | 14383 | 14404 | UAGUGCAGUUUUAAAACUAAACU | 768 |
| 1716981 | 690 | 711 | 14384 | 14405 | UCAGUGCAGUUUUAAAACUAAAC | 769 |
| 1716984 | 691 | 712 | 14385 | 14406 | UGCAGUGCAGUUUUAAAACUAAA | 770 |
| 1716993 | 1675 | 1696 | 15369 | 15390 | UUAUUUUCUCUUUCUUAUGAUUU | 771 |
| 1716999 | 1677 | 1698 | 15371 | 15392 | UUAUAUUUUCUCUUUCUUAUGAU | 772 |
| 1717002 | 1678 | 1699 | 15372 | 15393 | UAUAUAUUUUCUCUUUCUUAUGA | 773 |
| 1717005 | 1679 | 1700 | 15373 | 15394 | UAAUAUAUUUUCUCUUUCUUAUG | 774 |
| 1717011 | 1681 | 1702 | 15375 | 15396 | UUAAAUAUAUUUUCUCUUUCUUA | 775 |
| 1717014 | 1682 | 1703 | 15376 | 15397 | UGUAAAUAUAUUUUCUCUUUCUU | 776 |
| 1717020 | 1684 | 1705 | 15378 | 15399 | UUAGUAAAUAUAUUUUCUCUUUC | 777 |
| 1717023 | 1685 | 1706 | 15379 | 15400 | UAUAGUAAAUAUAUUUUCUCUUU | 778 |
| 1717026 | 1686 | 1707 | 15380 | 15401 | UAAUAGUAAAUAUAUUUUCUCUU | 779 |
| 1717032 | 1688 | 1709 | 15382 | 15403 | UUGAAUAGUAAAUAUAUUUUCUC | 780 |
| 1717035 | 1689 | 1710 | 15383 | 15404 | UAUGAAUAGUAAAUAUAUUUUCU | 781 |
| 1717047 | 1693 | 1714 | 15387 | 15408 | UUUUAAUGAAUAGUAAAUAUAUU | 782 |

Antisense oligonucleotides having the nucleobase sequences in Table B1, however, differing in the 3′terminal nucleoside or two 3′terminal nucleosides may also be prepared and used in the duplexes herein. For example, one or both of the 3′ terminal nucleoside is an inosine (having the nucleobase hypoxanthine designated as “I” in Table B1a). In another example, one or both of the 3′terminal nucleosides of the sequences in Table B1 is removed. Exemplary nucleobase sequences are depicted in Tables B1a and B1b:

TABLE Bla
Antisense oligonucleotides (antisense oligo)
targeted to human PLN

| SEQ ID NO: | NUCLEOBASE SEQUENCE |
|---|---|
| 1258 | TUUAAGAUGAGACAGAAAUUGAI |
| 1259 | TUUAAGAUGAGACAGAAAUUGII |
| 1260 | TUUAAGAUGAGACAGAAAUUGIA |
| 1261 | TAUAAAUAGAUUCUGUAGCUUAI |
| 1262 | TAUAAAUAGAUUCUGUAGCUUII |
| 1263 | TAUAAAUAGAUUCUGUAGCUUIA |
| 1264 | TAUAAATAGAUUCUGUAGCUUAI |
| 1265 | TAUAAATAGAUUCUGUAGCUUII |
| 1266 | TAUAAATAGAUUCUGUAGCUUIA |
| 1267 | TUAAGTUUUAGUCUUAAUCUUAI |
| 1268 | TUAAGTUUUAGUCUUAAUCUUII |
| 1269 | TUAAGTUUUAGUCUUAAUCUUIA |
| 1270 | TUAAGUTUUAGUCUUAAUCUUAI |
| 1271 | TUAAGUTUUAGUCUUAAUCUUII |
| 1272 | TUAAGUTUUAGUCUUAAUCUUIA |
| 1273 | TUUAAGAUGAGACAGAAAUUGA |
| 1274 | TUUAAGAUGAGACAGAAAUUGI |
| 1276 | TAUAAAUAGAUUCUGUAGCUUA |
| 1277 | TAUAAAUAGAUUCUGUAGCUUI |
| 1279 | TAUAAATAGAUUCUGUAGCUUA |
| 1280 | TAUAAATAGAUUCUGUAGCUUI |
| 1282 | TUAAGTUUUAGUCUUAAUCUUA |
| 1283 | TUAAGTUUUAGUCUUAAUCUUI |
| 1285 | TUAAGUTUUAGUCUUAAUCUUA |
| 1286 | TUAAGUTUUAGUCUUAAUCUUI |

TABLE B1b
Antisense oligonucleotides (antisense oligo)
targeted to human PLN

| SEQ ID NO: | NUCLEOBASE SEQUENCE |
|---|---|
| 1275 | TUUAAGAUGAGACAGAAAUUG |
| 1278 | TAUAAAUAGAUUCUGUAGCUU |
| 1281 | TAUAAATAGAUUCUGUAGCUU |
| 1284 | TUAAGTUUUAGUCUUAAUCUU |
| 1287 | TUAAGUTUUAGUCUUAAUCUU |

Design of Sense Oligonucleotides and Oligomeric Duplex Compounds that Target Human PLN

Sense oligonucleotides are listed in Table B2. Each sense oligonucleotide is 21 nucleosides in length having the nucleobase sequence in Table B2; has a sugar motif (from 5′ to 3′) of: yfyfffyyyyyyyyyy; wherein ‘y’ represents a 2′-OMe sugar moiety and the ‘f’ represents a 2′-fluoro sugar moiety; and an internucleoside linkage motif (from 5′ to 3′) of: ssooooooooooooooooss; wherein ‘o’ represents a phosphodiester internucleoside linkage and ‘s’ represents a phosphorothioate internucleoside linkage. Each antisense oligonucleotide in Table B1 is complementary to target nucleic acid PLN, and each sense oligonucleotide in Table B2 is complementary to the first 21 nucleosides of an antisense oligonucleotide (from 5′ to 3′) in Table B1 wherein the last two 3′-nucleosides of the antisense oligonucleotides are not paired with the sense oligonucleotide (are overhanging nucleosides). The oligomeric duplex compound with corresponding sense oligonucleotide (sense oligo.) compound and antisense oligonucleotide (antisense oligo.) compound are indicated by compound numbers, together with the sense oligonucleotide sequences in Table B2.

TABLE B2
Sense oligonucleotides and Duplex compounds
targeting human PLN

| | Anti- | | | |
|---|---|---|---|---|
| | sense | Sense | | |
| Duplex | Oligo. | Oligo. | | SEQ |
| Compound | Compound | Compound | Sense Oligo. Sequence | ID |
| Number | No. | No. | (5′ to 3′) | NO |
| 1716584 | 1716582 | 1716583 | CGUCAAAAGCUACAGAAUCUA | 783 |
| 1716596 | 1716594 | 1716595 | AAAAGCUACAGAAUCUAUUUA | 784 |
| 1716605 | 1716603 | 1716604 | AGCUACAGAAUCUAUUUAUCA | 785 |
| 1716608 | 1716606 | 1716607 | GCUACAGAAUCUAUUUAUCAA | 786 |
| 1716641 | 1716639 | 1716640 | UAUUUAUCAAUUUCUGUCUCA | 787 |
| 1716656 | 1716654 | 1716655 | AUCAAUUUCUGUCUCAUCUUA | 788 |
| 1716659 | 1716657 | 1716658 | UCAAUUUCUGUCUCAUCUUAA | 789 |
| 1716665 | 1716663 | 1716664 | AAUUUCUGUCUCAUCUUAAUA | 790 |
| 1716698 | 1716696 | 1716697 | AAAAGGUCAAGAUUAAGACUA | 791 |
| 1716701 | 1716699 | 1716700 | AAAGGUCAAGAUUAAGACUAA | 792 |
| 1716704 | 1716702 | 1716703 | AAGGUCAAGAUUAAGACUAAA | 793 |
| 1716707 | 1716705 | 1716706 | AGGUCAAGAUUAAGACUAAAA | 794 |
| 1716719 | 1716717 | 1716718 | CAAGAUUAAGACUAAAACUUA | 795 |
| 1716737 | 1716735 | 1716736 | UAAGACUAAAACUUAUUGUUA | 796 |
| 1716746 | 1716744 | 1716745 | GACUAAAACUUAUUGUUACCA | 797 |
| 1716752 | 1716750 | 1716751 | CUAAAACUUAUUGUUACCAUA | 798 |
| 1716773 | 1716771 | 1716772 | CAUAUGUAUUCAUCUGUUGGA | 799 |
| 1716797 | 1716795 | 1716796 | AUUCAUCUGUUGGAUCUUGUA | 800 |
| 1716800 | 1716798 | 1716799 | UUCAUCUGUUGGAUCUUGUAA | 801 |
| 1716803 | 1716801 | 1716802 | UCAUCUGUUGGAUCUUGUAAA | 802 |
| 1716809 | 1716807 | 1716808 | AUCUGUUGGAUCUUGUAAACA | 803 |
| 1716818 | 1716816 | 1716817 | UGUUGGAUCUUGUAAACAUGA | 804 |
| 1716821 | 1716819 | 1716820 | GUUGGAUCUUGUAAACAUGAA | 805 |
| 1716824 | 1716822 | 1716823 | UUGGAUCUUGUAAACAUGAAA | 806 |
| 1716827 | 1716825 | 1716826 | UGGAUCUUGUAAACAUGAAAA | 807 |
| 1716848 | 1716846 | 1716847 | AUUAACUUCAAAAUAAGUGUA | 808 |
| 1716857 | 1716855 | 1716856 | UAACUUCAAAAUAAGUGUAUA | 809 |
| 1716860 | 1716858 | 1716859 | AACUUCAAAAUAAGUGUAUAA | 810 |
| 1716863 | 1716861 | 1716862 | ACUUCAAAAUAAGUGUAUAAA | 811 |
| 1716866 | 1716864 | 1716865 | CUUCAAAAUAAGUGUAUAAAA | 812 |
| 1716878 | 1716876 | 1716877 | AAAAUAAGUGUAUAAAAUGCA | 813 |
| 1716881 | 1716879 | 1716880 | AAAUAAGUGUAUAAAAUGCAA | 814 |
| 1716902 | 1716900 | 1716901 | UGUAUAAAAUGCAACUGUUGA | 815 |
| 1716905 | 1716903 | 1716904 | CAAAUCUUUUCUGAAGAUGAA | 816 |
| 1716911 | 1716909 | 1716910 | AAUCUUUUCUGAAGAUGAAGA | 817 |
| 1716929 | 1716927 | 1716928 | UUUCUGAAGAUGAAGAGUUUA | 818 |
| 1716947 | 1716945 | 1716946 | AAGAUGAAGAGUUUAGUUUUA | 819 |
| 1716950 | 1716948 | 1716949 | AGAUGAAGAGUUUAGUUUUAA | 820 |
| 1716953 | 1716951 | 1716952 | GAUGAAGAGUUUAGUUUUAAA | 821 |
| 1716956 | 1716954 | 1716955 | AUGAAGAGUUUAGUUUUAAAA | 822 |
| 1716971 | 1716969 | 1716970 | GAGUUUAGUUUUAAAACUGCA | 823 |
| 1716989 | 1716987 | 1716988 | AGUUUUAAAACUGCACUGCCA | 824 |
| 1716992 | 1716990 | 1716991 | GUUUUAAAACUGCACUGCCAA | 825 |
| 1716998 | 1716996 | 1716997 | UCAUAAGAAAGAGAAAAUAUA | 826 |
| 1717010 | 1717008 | 1717009 | AAGAAAGAGAAAAUAUAUUUA | 827 |
| 1717019 | 1717017 | 1717018 | AAAGAGAAAAUAUAUUUACUA | 828 |
| 1717031 | 1717029 | 1717030 | AGAAAAUAUAUUUACUAUUCA | 829 |
| 1717040 | 1717038 | 1717039 | AAAUAUAUUUACUAUUCAUUA | 830 |
| 1717043 | 1717041 | 1717042 | AAUAUAUUUACUAUUCAUUAA | 831 |
| 1717046 | 1717044 | 1717045 | AUAUAUUUACUAUUCAUUAAA | 832 |
| 1716575 | 1716573 | 1716574 | GCACGUCAAAAGCUACAGAAA | 833 |
| 1716578 | 1716576 | 1716577 | CACGUCAAAAGCUACAGAAUA | 834 |
| 1716581 | 1716579 | 1716580 | ACGUCAAAAGCUACAGAAUCA | 835 |
| 1716587 | 1716585 | 1716586 | GUCAAAAGCUACAGAAUCUAA | 836 |
| 1716590 | 1716588 | 1716589 | UCAAAAGCUACAGAAUCUAUA | 837 |
| 1716593 | 1716591 | 1716592 | CAAAAGCUACAGAAUCUAUUA | 838 |
| 1716599 | 1716597 | 1716598 | AAAGCUACAGAAUCUAUUUAA | 839 |
| 1716602 | 1716600 | 1716601 | AAGCUACAGAAUCUAUUUAUA | 840 |
| 1716611 | 1716609 | 1716610 | CUACAGAAUCUAUUUAUCAAA | 841 |
| 1716614 | 1716612 | 1716613 | UACAGAAUCUAUUUAUCAAUA | 842 |
| 1716617 | 1716615 | 1716616 | ACAGAAUCUAUUUAUCAAUUA | 843 |
| 1716620 | 1716618 | 1716619 | CAGAAUCUAUUUAUCAAUUUA | 844 |
| 1716623 | 1716621 | 1716622 | AGAAUCUAUUUAUCAAUUUCA | 845 |
| 1716626 | 1716624 | 1716625 | GAAUCUAUUUAUCAAUUUCUA | 846 |
| 1716629 | 1716627 | 1716628 | AAUCUAUUUAUCAAUUUCUGA | 847 |
| 1716632 | 1716630 | 1716631 | AUCUAUUUAUCAAUUUCUGUA | 848 |
| 1716635 | 1716633 | 1716634 | UCUAUUUAUCAAUUUCUGUCA | 849 |
| 1716638 | 1716636 | 1716637 | CUAUUUAUCAAUUUCUGUCUA | 850 |
| 1716644 | 1716642 | 1716643 | AUUUAUCAAUUUCUGUCUCAA | 851 |
| 1716647 | 1716645 | 1716646 | UUUAUCAAUUUCUGUCUCAUA | 852 |
| 1716650 | 1716648 | 1716649 | UUAUCAAUUUCUGUCUCAUCA | 853 |
| 1716653 | 1716651 | 1716652 | UAUCAAUUUCUGUCUCAUCUA | 854 |
| 1716662 | 1716660 | 1716661 | CAAUUUCUGUCUCAUCUUAAA | 855 |
| 1716668 | 1716666 | 1716667 | AUUUCUGUCUCAUCUUAAUAA | 856 |
| 1716671 | 1716669 | 1716670 | UUUCUGUCUCAUCUUAAUAUA | 857 |
| 1716674 | 1716672 | 1716673 | UUCUGUCUCAUCUUAAUAUGA | 858 |
| 1716677 | 1716675 | 1716676 | UCUGUCUCAUCUUAAUAUGUA | 859 |
| 1716680 | 1716678 | 1716679 | CUGUCUCAUCUUAAUAUGUCA | 860 |
| 1716683 | 1716681 | 1716682 | UGUCUCAUCUUAAUAUGUCUA | 861 |
| 1716686 | 1716684 | 1716685 | GUCUCAUCUUAAUAUGUCUCA | 862 |
| 1716689 | 1716687 | 1716688 | UCUCAUCUUAAUAUGUCUCUA | 863 |
| 1716692 | 1716690 | 1716691 | CUCAUCUUAAUAUGUCUCUUA | 864 |
| 1716695 | 1716693 | 1716694 | GAAAAGGUCAAGAUUAAGACA | 865 |
| 1716710 | 1716708 | 1716709 | GGUCAAGAUUAAGACUAAAAA | 866 |
| 1716713 | 1716711 | 1716712 | GUCAAGAUUAAGACUAAAACA | 867 |
| 1716716 | 1716714 | 1716715 | UCAAGAUUAAGACUAAAACUA | 868 |
| 1716722 | 1716720 | 1716721 | AAGAUUAAGACUAAAACUUAA | 869 |
| 1716725 | 1716723 | 1716724 | AGAUUAAGACUAAAACUUAUA | 870 |
| 1716728 | 1716726 | 1716727 | GAUUAAGACUAAAACUUAUUA | 871 |
| 1716731 | 1716729 | 1716730 | AUUAAGACUAAAACUUAUUGA | 872 |
| 1716734 | 1716732 | 1716733 | UUAAGACUAAAACUUAUUGUA | 873 |
| 1716740 | 1716738 | 1716739 | AAGACUAAAACUUAUUGUUAA | 874 |
| 1716743 | 1716741 | 1716742 | AGACUAAAACUUAUUGUUACA | 875 |
| 1716749 | 1716747 | 1716748 | ACUAAAACUUAUUGUUACCAA | 876 |
| 1716755 | 1716753 | 1716754 | UGUUACCAUAUGUAUUCAUCA | 877 |
| 1716758 | 1716756 | 1716757 | GUUACCAUAUGUAUUCAUCUA | 878 |
| 1716761 | 1716759 | 1716760 | UUACCAUAUGUAUUCAUCUGA | 879 |
| 1716764 | 1716762 | 1716763 | UACCAUAUGUAUUCAUCUGUA | 880 |
| 1716767 | 1716765 | 1716766 | ACCAUAUGUAUUCAUCUGUUA | 881 |
| 1716770 | 1716768 | 1716769 | CCAUAUGUAUUCAUCUGUUGA | 882 |
| 1716776 | 1716774 | 1716775 | AUAUGUAUUCAUCUGUUGGAA | 883 |
| 1716779 | 1716777 | 1716778 | UAUGUAUUCAUCUGUUGGAUA | 884 |
| 1716782 | 1716780 | 1652838 | AUGUAUUCAUCUGUUGGAUCA | 885 |
| 1716785 | 1716783 | 1716784 | UGUAUUCAUCUGUUGGAUCUA | 886 |
| 1716788 | 1716786 | 1716787 | GUAUUCAUCUGUUGGAUCUUA | 887 |
| 1716794 | 1716792 | 1716793 | UAUUCAUCUGUUGGAUCUUGA | 888 |
| 1716806 | 1716804 | 1716805 | CAUCUGUUGGAUCUUGUAAAA | 889 |
| 1716812 | 1716810 | 1716811 | UCUGUUGGAUCUUGUAAACAA | 890 |
| 1716815 | 1716813 | 1716814 | CUGUUGGAUCUUGUAAACAUA | 891 |
| 1716830 | 1716828 | 1716829 | GGAUCUUGUAAACAUGAAAAA | 892 |
| 1716833 | 1716831 | 1716832 | GAUCUUGUAAACAUGAAAAGA | 893 |
| 1716836 | 1716834 | 1716835 | AUCUUGUAAACAUGAAAAGGA | 894 |
| 1716839 | 1716837 | 1716838 | UCUUGUAAACAUGAAAAGGGA | 895 |
| 1716842 | 1716840 | 1716841 | CUUGUAAACAUGAAAAGGGCA | 896 |
| 1716845 | 1716843 | 1716844 | AAUUAACUUCAAAAUAAGUGA | 897 |
| 1716851 | 1716849 | 1716850 | UUAACUUCAAAAUAAGUGUAA | 898 |
| 1716869 | 1716867 | 1716868 | UUCAAAAUAAGUGUAUAAAAA | 899 |
| 1716872 | 1716870 | 1716871 | UCAAAAUAAGUGUAUAAAAUA | 900 |
| 1716875 | 1716873 | 1716874 | CAAAAUAAGUGUAUAAAAUGA | 901 |
| 1716884 | 1716882 | 1716883 | AAUAAGUGUAUAAAAUGCAAA | 902 |
| 1716887 | 1716885 | 1716886 | AUAAGUGUAUAAAAUGCAACA | 903 |
| 1716890 | 1716888 | 1716889 | UAAGUGUAUAAAAUGCAACUA | 904 |
| 1716893 | 1716891 | 1716892 | AAGUGUAUAAAAUGCAACUGA | 905 |
| 1716896 | 1716894 | 1716895 | AGUGUAUAAAAUGCAACUGUA | 906 |
| 1716899 | 1716897 | 1716898 | GUGUAUAAAAUGCAACUGUUA | 907 |
| 1716908 | 1716906 | 1716907 | AAAUCUUUUCUGAAGAUGAAA | 908 |
| 1716917 | 1716915 | 1716916 | AUCUUUUCUGAAGAUGAAGAA | 909 |
| 1716920 | 1716918 | 1716919 | UCUUUUCUGAAGAUGAAGAGA | 910 |
| 1716923 | 1716921 | 1716922 | CUUUUCUGAAGAUGAAGAGUA | 911 |
| 1716926 | 1716924 | 1716925 | UUUUCUGAAGAUGAAGAGUUA | 912 |
| 1716932 | 1716930 | 1716931 | UUCUGAAGAUGAAGAGUUUAA | 913 |
| 1716935 | 1716933 | 1716934 | UCUGAAGAUGAAGAGUUUAGA | 914 |
| 1716938 | 1716936 | 1716937 | CUGAAGAUGAAGAGUUUAGUA | 915 |
| 1716941 | 1716939 | 1716940 | UGAAGAUGAAGAGUUUAGUUA | 916 |
| 1716944 | 1716942 | 1716943 | GAAGAUGAAGAGUUUAGUUUA | 917 |
| 1716959 | 1716957 | 1716958 | UGAAGAGUUUAGUUUUAAAAA | 918 |
| 1716962 | 1716960 | 1716961 | GAAGAGUUUAGUUUUAAAACA | 919 |
| 1716965 | 1716963 | 1716964 | AAGAGUUUAGUUUUAAAACUA | 920 |
| 1716968 | 1716966 | 1716967 | AGAGUUUAGUUUUAAAACUGA | 921 |
| 1716974 | 1716972 | 1716973 | AGUUUAGUUUUAAAACUGCAA | 922 |
| 1716977 | 1716975 | 1716976 | GUUUAGUUUUAAAACUGCACA | 923 |
| 1716980 | 1716978 | 1716979 | UUUAGUUUUAAAACUGCACUA | 924 |
| 1716983 | 1716981 | 1716982 | UUAGUUUUAAAACUGCACUGA | 925 |
| 1716986 | 1716984 | 1716985 | UAGUUUUAAAACUGCACUGCA | 926 |
| 1716995 | 1716993 | 1716994 | AUCAUAAGAAAGAGAAAAUAA | 927 |
| 1717001 | 1716999 | 1717000 | CAUAAGAAAGAGAAAAUAUAA | 928 |
| 1717004 | 1717002 | 1717003 | AUAAGAAAGAGAAAAUAUAUA | 929 |
| 1717007 | 1717005 | 1717006 | UAAGAAAGAGAAAAUAUAUUA | 930 |
| 1717013 | 1717011 | 1717012 | AGAAAGAGAAAAUAUAUUUAA | 931 |
| 1717016 | 1717014 | 1717015 | GAAAGAGAAAAUAUAUUUACA | 932 |
| 1717022 | 1717020 | 1717021 | AAGAGAAAAUAUAUUUACUAA | 933 |
| 1717025 | 1717023 | 1717024 | AGAGAAAAUAUAUUUACUAUA | 934 |
| 1717028 | 1717026 | 1717027 | GAGAAAAUAUAUUUACUAUUA | 935 |
| 1717034 | 1717032 | 1717033 | GAAAAUAUAUUUACUAUUCAA | 936 |
| 1717037 | 1717035 | 1717036 | AAAAUAUAUUUACUAUUCAUA | 937 |
| 1717049 | 1717047 | 1717048 | UAUAUUUACUAUUCAUUAAAA | 938 |

### Activity of Oligomeric Duplex Compounds Targeting Human PLN

Cultured iCell® cardiomyocytes (FujiFilm Cellular Dynamics, Inc.; Catalog No: R1017) were treated with compound at a concentration of 125 nM using Lipofectamine RNAiMAX at a density of 8,000 cells per well; or cultured A431 cells were treated with compound at a concentration of 100 nM using Lipofectamine RNAiMAX at a density of 10,000 cells per well. After a treatment period of 24 hours (for cardiomyocytes) or 72 hours (for A431 cells), total RNA was isolated from the cells and PLN RNA levels were measured by quantitative real-time RTPCR. PLN RNA levels were measured by human primer-probe set RTS40403 (forward sequence GAAGAACAGAAGGGCAGAGATT, designated herein as SEQ ID NO: 1046; reverse sequence AAACCAGTGAGGTGAGTGTATC, designated herein as SEQ ID NO: 1047; probe sequence TTTCCAGCTTGTAGATGAGGCGTCAC, designated herein as SEQ ID NO: 1048) and human primer-probe set RTS40407 (forward sequence GCTGCCAAGGCTACCTAAA, designated herein as SEQ ID NO: 1049; reverse sequence GAGTGAGGTATTGGACTTTCTCC, designated herein as SEQ ID NO: 1050; probe sequence TCAGACTTCCTGTCCTGCTGGTATCA, designated herein as SEQ ID NO: 1051). PLN RNA levels were normalized to total RNA content, as measured by RIBOGREEN®. Reduction of PLN RNA is presented as percent PLN RNA relative to the amount of PLN RNA in untreated control cells (% UTC). Results from various experiments are presented in Table B3.

TABLE B3
Effect of oligomeric duplex compounds
targeting human PLN in Cells

| | PLN RNA in IPS derived | | PLN RNA in A431 | |
|---|---|---|---|---|
| Compound | cardiomyocytes (% UTC) | | cells (% UTC) | |
| No. | RTS40403 | RTS40407 | RTS40403 | RTS40407 |
| 1716812 | 6 | 8 | 13 | 12 |
| 1716815 | 4 | 5 | 19 | 13 |
| 1716818 | 5 | 6 | 34 | 28 |
| 1716821 | 12 | 12 | 22 | 19 |
| 1716824 | 5 | 6 | 20 | 18 |
| 1716827 | 5 | 7 | 15 | 12 |
| 1716830 | 6 | 8 | 19 | 17 |
| 1716833 | 6 | 8 | 19 | 16 |
| 1716836 | 5 | 7 | 23 | 21 |
| 1716839 | 5 | 7 | 24 | 23 |
| 1716842 | 6 | 6 | 25 | 19 |
| 1716845 | 7 | 8 | 21 | 20 |
| 1716848 | 4 | 5 | 31 | 28 |
| 1716851 | 8 | 10 | 39 | 35 |
| 1716857 | 5 | 6 | 24 | 21 |
| 1716860 | 6 | 9 | 24 | 23 |
| 1716863 | 9 | 12 | 28 | 29 |
| 1716866 | 4 | 8 | 22 | 24 |
| 1716869 | 6 | 11 | 38 | 42 |
| 1716872 | 5 | 6 | 25 | 24 |
| 1716875 | 3 | 5 | 28 | 23 |
| 1716878 | 11 | 12 | 32 | 27 |
| 1716881 | 6 | 9 | 26 | 25 |
| 1716884 | 12 | 12 | 30 | 24 |
| 1716887 | 9 | 11 | 22 | 22 |
| 1716890 | 9 | 9 | 24 | 18 |
| 1716893 | 8 | 11 | 22 | 22 |
| 1716896 | 7 | 11 | 31 | 31 |
| 1716899 | 9 | 13 | 24 | 28 |
| 1716902 | 5 | 6 | 24 | 20 |
| 1716905 | 9 | 17 | 65 | 69 |
| 1716908 | 8 | 27 | 27 | 37 |
| 1716911 | 9 | 25 | 39 | 48 |
| 1716917 | 8 | 26 | 35 | 57 |
| 1716920 | 7 | 25 | 28 | 43 |
| 1716923 | 12 | 21 | 22 | 30 |
| 1716926 | 5 | 19 | 21 | 37 |
| 1716929 | 11 | 40 | 26 | 43 |
| 1716932 | 22 | 44 | 39 | 62 |
| 1716935 | 5 | 13 | 18 | 28 |
| 1716938 | 4 | 14 | 23 | 35 |
| 1716941 | 4 | 18 | 35 | 51 |
| 1716944 | 7 | 27 | 21 | 35 |
| 1716947 | 5 | 18 | 26 | 43 |
| 1716950 | 6 | 23 | 22 | 41 |
| 1716953 | 7 | 22 | 16 | 25 |
| 1716956 | 9 | 24 | 19 | 32 |
| 1716959 | 7 | 29 | 17 | 30 |
| 1716962 | 6 | 22 | 21 | 28 |
| 1716965 | 7 | 20 | 53 | 56 |
| 1716968 | 7 | 16 | 51 | 58 |
| 1716971 | 13 | 30 | 30 | 41 |
| 1716974 | 26 | 45 | 18 | 30 |
| 1716977 | 40 | 40 | 51 | 65 |
| 1716980 | 12 | 26 | 33 | 39 |
| 1716983 | 10 | 27 | 44 | 59 |
| 1716986 | 15 | 46 | 34 | 48 |
| 1716989 | 15 | 36 | 25 | 36 |
| 1716992 | 42 | 49 | 38 | 41 |
| 1716995 | 6 | 37 | 24 | 45 |
| 1716998 | 8 | 34 | 26 | 42 |
| 1717001 | 8 | 53 | 20 | 48 |
| 1717004 | 9 | 41 | 22 | 39 |
| 1717007 | 9 | 57 | 22 | 42 |
| 1717010 | 10 | 77 | 25 | 44 |
| 1717013 | 5 | 35 | 20 | 37 |
| 1717016 | 8 | 57 | 14 | 34 |
| 1717019 | 8 | 66 | 16 | 39 |
| 1717022 | 7 | 51 | 16 | 34 |
| 1717025 | 5 | 33 | 23 | 47 |
| 1717028 | 7 | 47 | 17 | 34 |
| 1717031 | 8 | 48 | 39 | 48 |
| 1717034 | 6 | 36 | 20 | 33 |
| 1717037 | 17 | 52 | 57 | 58 |
| 1717040 | 5 | 36 | 20 | 32 |
| 1717043 | 5 | 46 | 27 | 48 |
| 1717046 | 10 | 38 | 23 | 43 |
| 1717049 | 7 | 46 | 22 | 35 |
| 1716575 | 10 | 10 | 42 | 29 |
| 1716578 | 4 | 3 | 25 | 16 |
| 1716581 | 6 | 2 | 30 | 22 |
| 1716584 | 5 | 3 | 22 | 13 |
| 1716587 | 6 | 4 | 18 | 10 |
| 1716590 | 7 | 5 | 31 | 20 |
| 1716593 | 6 | 5 | 33 | 34 |
| 1716596 | 15 | 13 | 30 | 31 |
| 1716599 | 14 | 12 | 56 | 56 |
| 1716602 | 3 | 2 | 20 | 16 |
| 1716605 | 348 | 3 | 21 | 14 |
| 1716608 | 750085 | 3 | 19 | 11 |
| 1716611 | 5 | 4 | 32 | 24 |
| 1716614 | 4 | 3 | 29 | 21 |
| 1716617 | 6 | 6 | 29 | 25 |
| 1716620 | 9 | 8 | 21 | 16 |
| 1716623 | 5 | 4 | 18 | 12 |
| 1716626 | 4 | 3 | 20 | 13 |
| 1716629 | 10 | 8 | 52 | 57 |
| 1716632 | 5 | 4 | 45 | 37 |
| 1716635 | 4 | 3 | 71 | 68 |
| 1716638 | 7 | 4 | 35 | 22 |
| 1716641 | 5 | 4 | 37 | 35 |
| 1716644 | 6 | 4 | 23 | 17 |
| 1716647 | 6 | 5 | 34 | 28 |
| 1716650 | 6 | 3 | 35 | 26 |
| 1716653 | 17 | 9 | 37 | 37 |
| 1716656 | 11 | 8 | 39 | 39 |
| 1716659 | 11 | 9 | 41 | 35 |
| 1716662 | 3 | 2 | 20 | 15 |
| 1716665 | 12 | 10 | 36 | 37 |
| 1716668 | 16 | 12 | 37 | 24 |
| 1716671 | 8 | 4 | 32 | 24 |
| 1716674 | 11 | 9 | 30 | 19 |
| 1716677 | 10 | 8 | 34 | 29 |
| 1716680 | 8 | 5 | 21 | 15 |
| 1716683 | 113 | 93 | 62 | 63 |
| 1716686 | 41 | 32 | 54 | 57 |
| 1716689 | 10 | 7 | 30 | 21 |
| 1716692 | 6 | 4 | 21 | 13 |
| 1716695 | 5 | 4 | 31 | 25 |
| 1716698 | 13 | 11 | 65 | 58 |
| 1716701 | 8 | 8 | 28 | 20 |
| 1716704 | 5 | 4 | 30 | 21 |
| 1716707 | 22 | 20 | 24 | 20 |
| 1716710 | 8 | 8 | 19 | 14 |
| 1716713 | 7 | 7 | 25 | 21 |
| 1716716 | 8 | 9 | 38 | 33 |
| 1716719 | 7 | 8 | 36 | 33 |
| 1716722 | 6 | 6 | 29 | 19 |
| 1716725 | 5 | 5 | 58 | 60 |
| 1716728 | 11 | 10 | 65 | 65 |
| 1716731 | 5 | 7 | 21 | 15 |
| 1716734 | 8 | 12 | 21 | 17 |
| 1716737 | 7 | 9 | 39 | 31 |
| 1716740 | 9 | 9 | 22 | 18 |
| 1716743 | 8 | 9 | 26 | 19 |
| 1716746 | 8 | 11 | 31 | 22 |
| 1716749 | 10 | 12 | 29 | 18 |
| 1716752 | 5 | 7 | 20 | 15 |
| 1716755 | 8 | 9 | 33 | 19 |
| 1716758 | 11 | 11 | 34 | 25 |
| 1716761 | 23 | 23 | 33 | 26 |
| 1716764 | 9 | 10 | 26 | 17 |
| 1716767 | 9 | 11 | 28 | 23 |
| 1716770 | 9 | 12 | 20 | 15 |
| 1716773 | 9 | 11 | 19 | 13 |
| 1716776 | 8 | 12 | 21 | 17 |
| 1716779 | 13 | 16 | 25 | 14 |
| 1716782 | 14 | 15 | 35 | 22 |
| 1716785 | 13 | 14 | 22 | 14 |
| 1716788 | 7 | 11 | 22 | 14 |
| 1716794 | 10 | 13 | 24 | 14 |
| 1716797 | 9 | 13 | 36 | 24 |
| 1716800 | 24 | 23 | 31 | 16 |
| 1716803 | 10 | 13 | 29 | 18 |
| 1716806 | 10 | 12 | 22 | 14 |
| 1716809 | 14 | 14 | 21 | 12 |

### Transgenic Animals

The following transgenic mice were used to determine effects of the modified oligonucleotides and oligomeric duplex compounds provided herein on human PLN:

hPLN^{tg/tg} knock-in mice (Taconic Biosciences): The human PLN allele (NCBI transcript NM_002667.4) harboring the p.Arg14del mutation was generated by CRISPR/Cas9-mediated gene editing. The mutant human allele was inserted into mouse PLN (NCBI transcript NM_023129.5) to generate constitutive humanization of the PIn gene. The mouse genomic sequence from exon 1 including the 5′ UTR to 29 bp downstream of exon 2 including the 3′ UTR was replaced with the human counterpart. A plasmid containing homology regions of the mouse PLN gene and the replaced human region including the R14del mutation was co-transfected with a plasmid allowing the expression of Cas9 mRNA, the specific gRNA and puromycin N acetyl transferase (puromycin resistance) into the Taconic C57BL/6N Tac ES cell line. Heterozygous mice generated are called hPLN^{tg/+} herein. Homozygous mice generated are called hPLN^{tg/tg} herein.

hTfR^{+/-} knock-in mice were used to determine effects of the modified oligonucleotides described below on mouse PLN. Human transferrin receptor (hTFR)/CD71 knock-in mice used in these studies have the coding region of mouse exon 2 as well as the splice donor-site of mouse intron 2 replaced with the human TFR open reading frame according to NCBI transcript NM_001128148.2. Humanization of the transferrin receptor gene was done via CRISPR/Cas-9-mediated gene editing, allowing for generation of a model with constitutive expression of humanized transferrin receptor gene. Targeting strategy was based on NCBI transcripts NM_011638.4 (mouse) and NM_001128148.2 (human). A plasmid allowing expression of Cas9 mRNA, specific gRNA, and the puromycin resistance cassette; and a plasmid containing the homology regions of the mouse transferrin receptor gene, an FRT site, and the replaced human region were co-transfected into the Taconic Biosciences C57BL/6N Tac ES cell line. The humanized mice are called hTfR^{+/-} knock-in mice herein. They express one copy of the mouse TFR gene and one copy of the humanized TFR gene under the control of the endogenous mouse promoter.

hTfR^{tg/+}/hPLN^{tg/tg} knock-in mice were generated by crossing hTfR^{tg/+} and hPLN^{tg/tg}. These mice are heterozygous for hTfr and homozygous for human PLN.

hTfR^{tg/+}/hPLN^{tg/+} mice were generated by crossing hTfR^{tg/+} and hPLN^{tg/+}. These mice are heterozygous for hTfr and heterozygous for human PLN.

### Example 1: Design of Modified Oligomeric Compounds that Target Human PLN

Oligomeric compounds comprising antisense oligonucleotides complementary to a human PLN nucleic acid, and sense oligonucleotides complementary to the antisense oligonucleotides were designed as follows.

### Design of Antisense Oligonucleotides

Antisense oligonucleotides having nucleobase sequences in Tables 1-9 below are 23 (Tables 1-6), or 22 (Tables 8 and 9), or 21 (Table 7) nucleosides in length, have a sugar motif as designated in the column labeled “Antisense Strand Sugar Motif (5′ to 3′),” wherein each ‘y’ represents a 2′-OMe sugar moiety, each ‘f’ represents a 2′-F sugar moiety, each ‘[FHNA]’ represents a 3′-fluoro-hexitol sugar moiety, each ‘d’ represents a 2′-deoxy sugar moiety, and each ‘e’ represents a 2′-MOE sugar moiety. In Table 1, each antisense oligonucleotide has an internucleoside linkage motif (from 5′ to 3′) of: ssooooooooooooooooooss, in Table 2 each antisense oligonucleotide has an internucleoside linkage motif (from 5′ to 3′) of: ssooosooooooooooooooss, in Table 3 each antisense oligonucleotide has an internucleoside linkage motif (from 5′ to 3′) of: ssooooooooooooooooooss, in Table 4 each antisense oligonucleotide has an internucleoside linkage motif (from 5′ to 3′) of: ssooooooooooooooooooss, in Table 5 each antisense oligonucleotide has an internucleoside linkage motif (from 5′ to 3′) of: ssoososoooooooooooooss, in Table 6 each antisense oligonucleotide has an internucleoside linkage motif (from 5′ to 3′) of: ssooooooooooooooooooss, in Table 7 each antisense oligonucleotide has an internucleoside linkage motif (from 5′ to 3′) of: ssooooooooooooooooss, in Table 8 each antisense oligonucleotide has an internucleoside linkage motif (from 5′ to 3′) of ssoooooooooooooooooos, and in Table 9 each antisense oligonucleotide has an internucleoside linkage motif (from 5′ to 3′) of: ssoooooooooooooooooos; wherein each ‘o’ represents a phosphodiester internucleoside linkage and each ‘s’ represents a phosphorothioate internucleoside linkage. Each cytosine residue is a non-methylated cytosine. Each antisense oligonucleotide has a vinyl phosphonate (vP-) group on the 5′-end.

In each of Tables 1-9, “Start site” indicates the 5′-most nucleoside to which the antisense oligonucleotide is complementary in the target nucleic acid sequence. “Stop site” indicates the 3′-most nucleoside to which the antisense oligonucleotide is complementary in the target nucleic acid sequence.

Each antisense oligonucleotide listed in Tables 1-9 is complementary to SEQ ID NO: 1 (GENBANK Accession No. NM_002667.4), except for a single mismatch at position 1 on the 5′ end of the antisense oligonucleotide in each of Tables 1-9; and a second mismatch at position 23 on the 3′-end of the antisense oligonucleotides in Table 3, a second mismatch at position 22 on the 3′-end of the antisense oligonucleotides in Table 4, a second mismatch at position 23 on the 3′-end of the antisense oligonucleotides in Table 5, two mismatches at positions 22 and 23 on the 3′-end of the antisense oligonucleotides in Table 6, two mismatches at positions 20 and 21 on the 3′-end of the antisense oligonucleotides in Table 7, and a second mismatch at position 22 on the 3′-end of the antisense oligonucleotides in Table 8.

TABLE 1
Antisense oligonucleotides targeted to human PLN

| | SEQ | NO: 1 | | | |
|---|---|---|---|---|---|
| | ID | SEQ ID | | | |
| Compound | NO: 1 | Stop | Antisense Strand Sequence | Antisense Strand Sugar Motif | SEQ |
| No. | Start Site | Site | (5′ to 3′) | (5′ to 3′) | ID NO |
| 1652826 | 324 | 345 | TUUAAGAUGAGACAGAAAUUGAU | efyyyfyyyyyyyfyfyyyyyyy | 939 |
| 1652709 | 304 | 325 | TAUAAAUAGAUUCUGUAGCUUUU | efyyyfyyyyyyyfyfyyyyyyy | 940 |
| 1691449 | 1044 | 1065 | TUUCUUUGAAAAAAUUACUUAAA | efyyyfyyyyyyyfyfyyyyyyy | 941 |
| 1691837 | 514 | 535 | TUAAGUUUUAGUCUUAAUCUUGA | efyyyfyyyyyyyfyfyyyyyyy | 942 |
| 1692941 | 554 | 575 | TAUGUUUACAAGAUCCAACAGAU | efyyyfyyyyyyyfyfyyyyyyy | 943 |
| 1692943 | 604 | 625 | TCAUUUUAUACACUUAUUUUGAA | efyyyfyyyyyyyfyfyyyyyyy | 944 |
| 1692945 | 684 | 705 | TAGUUUUAAAACUAAACUCUUCA | efyyyfyyyyyyyfyfyyyyyyy | 945 |
| 1692950 | 674 | 695 | TCUAAACUCUUCAUCUUCAGAAA | efyyyfyyyyyyyfyfyyyyyyy | 946 |
| 1692952 | 1684 | 1705 | TUAGUAAAUAUAUUUUCUCUUUC | efyyyfyyyyyyyfyfyyyyyyy | 947 |
| 1731386 | 324 | 345 | TUUAAGAUGAGACAGAAAUUGAT | efyyyfyyyyyyyfyfyyyyyee | 948 |
| 1731388 | 324 | 345 | TUUAAGAUGAGACAGAAAUUGAT | e[FHNA]yyyyyyyyyyy[FHNA]yyyyyyyee | 949 |
| 1731387 | 324 | 345 | TUUAAGAUGAGACAGAAAUUGAT | efyyyyyyyyyyyfyyyyyyyee | 950 |
| 1731389 | 324 | 345 | TUUAAGAUGAGACAGAAAUUGAT | e[FHNA]yyyfyyyyyyyfyfyyyyyee | 951 |
| 1731390 | 324 | 345 | TUUAAGAUGAGACAGAAAUUGAT | efyyydyyyyyyyfyfyyyyyee | 952 |
| 1736402 | 324 | 345 | TUUAAGAUGAGACAGAAAUUGAU | e[FHNA]yyyfyyyyyyyfyfyyyyyyy | 953 |
| 1736403 | 324 | 345 | TUUAAGAUGAGACAGAAAUUGAU | efyyydyyyyyyydydyyyyyyy | 954 |
| 1736404 | 324 | 345 | TUUAAGAUGAGACAGAAAUUGAU | efyyydyyyyyyyfyfyyyyyyy | 955 |
| 1738170 | 324 | 345 | TUUAAGAUGAGACAGAAAUUGAU | efyydydyyyyyyfyfyyyyyyy | 956 |
| 1738171 | 324 | 345 | TUUAAGAUGAGACAGAAAUUGAT | efyyyfyyyyyyyfyyyyyyyee | 957 |
| 1738172 | 324 | 345 | TUUAAGAUGAGACAGAAAUUGAT | efyyyyyyyyyyyfyfyyyyyee | 958 |
| 1738173 | 324 | 345 | TUUAAGAUGAGACAGAAAUUGAU | efyyyfyyyyyyyfyyyyyyyyy | 959 |
| 1738174 | 324 | 345 | TUUAAGAUGAGACAGAAAUUGAU | efyyyyyyyyyyyfyfyyyyyyy | 960 |
| 1749977 | 324 | 345 | TUUAAGAUGAGACAGAAAUUGAU | e[FHNA]yydydyyyyfyfyfyyyyyyy | 961 |
| 1744533 | 324 | 345 | TUUAAGAUGAGACAGAAAUUGAU | efyyyfyyyyyyyfydyyyyyyy | 962 |
| 1744534 | 324 | 345 | TUUAAGAUGAGACAGAAAUUGAT | efyyyfyyyyyyyfydyyyyyee | 963 |
| 1749975 | 324 | 345 | TUUAAGAUGAGACAGAAAUUGAU | efyyyfyyyyyyyfyfyyyyyey | 964 |
| 1801601 | 304 | 325 | TAUAAATAGAUUCUGUAGCUUII | efyydydyyyyyyfyfyyyyyee | 1265 |

TABLE 2
Antisense oligonucleotide targeted to human PLN

| | SEQ | SEQ | | | |
|---|---|---|---|---|---|
| | ID | ID | | | |
| | NO: 1 | NO: 1 | | | |
| Compound | Start | Stop | Antisense Strand Sequence | Antisense Strand Sugar Motif | SEQ |
| No. | Site | Site | (5′ to 3′) | (5′ to 3′) | ID NO |
| 1731397 | 324 | 345 | TUUAAGAUGAGACAGAAAUUGAT | efyyydyyyyyyyfyfyyyyyee | 965 |

TABLE 3
Antisense oligonucleotides targeted to human PLN

| | SEQ | SEQ | | | |
|---|---|---|---|---|---|
| | ID | ID | | | |
| | NO: 1 | NO: 1 | | | |
| Compound | Start | Stop | Antisense Strand Sequence | Antisense Strand Sugar Motif | SEQ |
| No. | Site | Site | (5′ to 3′) | (5′ to 3′) | ID NO |
| 1749976 | 325 | 345 | TUUAAGAUGAGACAGAAAUUGAA | efyyyfyyyyyyyfyfyyyyyey | 966 |
| 1746643 | 325 | 345 | TUUAAGAUGAGACAGAAAUUGAA | efyyydyyyyyyyfyfyyyyyee | 967 |
| 1746644 | 325 | 345 | TUUAAGAUGAGACAGAAAUUGAA | efyydydyyyyyyfyfyyyyyee | 968 |
| 1749974 | 325 | 345 | TUUAAGAUGAGACAGAAAUUGAA | efyyyyyyyyyyyfyfyyyyyee | 969 |
| 1736400 | 325 | 345 | TUUAAGAUGAGACAGAAAUUGAA | efyyyfyyyyyyyfyfyyyyyee | 970 |

TABLE 4
Antisense oligonucleotides targeted to human PLN

| | SEQ | SEQ | | | |
|---|---|---|---|---|---|
| Antisense | ID | ID | | | |
| Oligo. | NO: 1 | NO: 1 | | | |
| Compound | Start | Stop | Antisense Strand Sequence | Antisense Strand Sugar Motif | SEQ |
| No. | Site | Site | (5′ to 3′) | (5′ to 3′) | ID NO |
| 1753977 | 516 | 535 | TUAAGTUUUAGUCUUAAUCUUAA | efyyydyyyyyyyfyfyyyyyee | 971 |
| 1753978 | 516 | 535 | TUAAGUTUUAGUCUUAAUCUUAA | efyydydyyyyyyfyfyyyyyee | 972 |
| 1801600 | 306 | 325 | TAUAAATAGAUUCUGUAGCUUAI | efyydydyyyyyyfyfyyyyyee | 1264 |

TABLE 5
Antisense oligonucleotide targeted to human PLN

| | SEQ | SEQ | | | |
|---|---|---|---|---|---|
| | ID | ID | | | |
| | NO: 1 | NO: 1 | | | |
| Compound | Start | Stop | Antisense Strand Sequence | Antisense Strand Sugar Motif | SEQ |
| No. | Site | Site | (5′ to 3′) | (5′ to 3′) | ID NO |
| 1746645 | 325 | 345 | TUUAAGAUGAGACAGAAAUUGAA | efyydydyyyyyyfyfyyyyyee | 973 |

TABLE 6
Antisense oligonucleotides targeted to human PLN

| | SEQ | SEQ | | | |
|---|---|---|---|---|---|
| | ID | ID | | | |
| | NO: 1 | NO: 1 | | | |
| Compound | Start | Stop | Antisense Strand Sequence | Antisense Strand Sugar | SEQ |
| No. | Site | Site | (5′ to 3′) | Motif (5′ to 3′) | ID NO |
| 1753975 | 306 | 325 | TAUAAAUAGAUUCUGUAGCUUAA | efyyydyyyyyyyfyfyyyyyee | 974 |
| 1753976 | 306 | 325 | TAUAAATAGAUUCUGUAGCUUAA | efyydydyyyyyyfyfyyyyyee | 975 |
| 1753981 | 306 | 325 | TAUAAAUAGAUUCUGUAGCUUAA | efyyyyyyyyyyyfyfyyyyyee | 1254 |

TABLE 7
Antisense oligonucleotide targeted to human PLN

| | SEQ | SEQ | | | |
|---|---|---|---|---|---|
| | ID | ID | | | |
| | NO: 1 | NO: 1 | | | |
| Compound | Start | Stop | Antisense Strand Sequence | Antisense Strand Sugar | SEQ |
| No. | Site | Site | (5′ to 3′) | Motif (5′ to 3′) | ID NO |
| 1736401 | 328 | 345 | TUUAAGAUGAGACAGAAAUAA | efyyyfyyyyyyyfyfyyyee | 976 |

TABLE 8
Antisense oligonucleotide targeted to human PLN

| | SEQ | SEQ | | | |
|---|---|---|---|---|---|
| | ID | ID | | | |
| | NO: 1 | NO: 1 | | | |
| Compound | Start | Stop | Antisense Strand Sequence | Antisense Strand Sugar Motif | SEQ |
| No. | Site | Site | (5′ to 3′) | (5′ to 3′) | ID NO |
| 1805817 | 306 | 325 | TAUAAATAGAUUCUGUAGCUUA | efyydydyyyyyyfyfyyyyye | 1279 |

TABLE 9
Antisense oligonucleotide targeted to human PLN

| | SEQ | SEQ | | | |
|---|---|---|---|---|---|
| | ID | ID | | | |
| | NO: 1 | NO: 1 | | | |
| Compound | Start | Stop | Antisense Strand Sequence | Antisense Strand Sugar Motif | SEQ |
| No. | Site | Site | (5′ to 3′) | (5′ to 3′) | ID NO |
| 1801602 | 305 | 325 | TAUAAATAGAUUCUGUAGCUUI | efyydydyyyyyyfyfyyyyye | 1280 |

### Design of Sense Oligonucleotides

The sense oligonucleotide in each case having the nucleobase sequence in Tables 10-15 below is 19-21 nucleosides in length. Each sense oligonucleotide has a sugar motif as designated in the column labeled “Sense Strand Sugar Motif (5′ to 3′); wherein each ‘y’ represents a 2′-OMe sugar moiety, each ‘d’ represents a 2′-deoxy sugar moiety, each ‘e’ represents a 2′-MOE sugar moiety, and each ‘f’ represents a 2′-F sugar moiety; and an internucleoside linkage motif as designated in the column labeled “Sense Strand Internucleoside Linkage (5′ to 3′)”; wherein each ‘o’ represents a phosphodiester internucleoside linkage, each ‘z’ represents a mesyl phosphoramidate internucleoside linkage, and each ‘s’ represents a phosphorothioate internucleoside linkage. Each cytosine residue is non-methylated unless otherwise indicated. 5-methylcytosines are represented in bold underlined italicized font as “C”. Each sense oligonucleotide is complementary to an antisense oligonucleotide in one or more of Tables 1-9.

Each sense oligonucleotide in Table 10 is conjugated to a 2-(hydroxymethyl)-6-palmitamidohexyl phosphoryl conjugate group attached to the 3′-OH of the oligonucleotide. Each sense oligonucleotide in Table 11 and in Table 12 is conjugated to a 6-palmitamidohexyl phosphoryl conjugate group attached to the 5′-OH of the oligonucleotide. Each sense oligonucleotide in Table 13 is conjugated to a 6-aminohexyl phosphoryl conjugate group attached to the 5′-OH of the oligonucleotide. Each sense oligonucleotide in Table 14 is conjugated at the 3′-end of the sense oligonucleotide to a bicycle ligand, BCY17901. Each sense oligonucleotide in Table 15 is conjugated at the 5′-end of the sense oligonucleotide to a bicycle ligand, BCY17901.

In Table 10, the structure for the 6-palmitamidohexyl phosphoryl conjugate group attached to the 3′-OH of the oligonucleotide is:

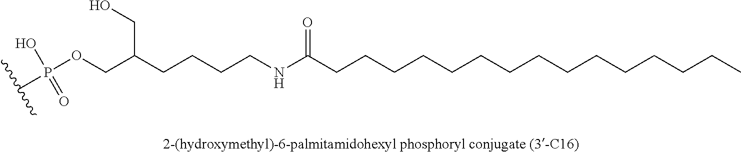 [SMILES: *P(=O)(O)OCC(CO)CCCCNC(=O)CCCCCCCCCCCCCCC]

TABLE 10
3′-2-(hydroxymethyl)-6-palmitamidohexyl phosphoryl conjugated sense oligonucleotides

| | | | Sense Strand | SEQ |
|---|---|---|---|---|
| Compound | Sense Oligo. Sequence | Sense Strand Sugar Motif | Internucleoside Linkage | ID |
| No. | (5′ to 3′) | (5′ to 3′) | (5′ to 3′) | NO |
| 1652825 | AAGCUACAGAAUCUAUUUAUA | yyyyyyfyfffyyyyyyyyyy | ssooooooooooooooooss | 977 |
| 1652841 | CAAUUUCUGUCUCAUCUUAAA | yyyyyyfyfffyyyyyyyyyy | ssooooooooooooooooss | 978 |
| 1691836 | UAAGUAAUUUUUUCAAAGAAA | yyyyyyfyfffyyyyyyyyyy | ssooooooooooooooooss | 979 |
| 1692947 | AAGAUUAAGACUAAAACUUAA | yyyyyyfyfffyyyyyyyyyy | ssooooooooooooooooss | 980 |
| 1692948 | CUGUUGGAUCUUGUAAACAUA | yyyyyyfyfffyyyyyyyyyy | ssooooooooooooooooss | 981 |
| 1692949 | CAAAAUAAGUGUAUAAAAUGA | yyyyyyfyfffyyyyyyyyyy | ssooooooooooooooooss | 982 |
| 1692946 | AAGAGUUUAGUUUUAAAACUA | yyyyyyfyfffyyyyyyyyyy | ssooooooooooooooooss | 983 |
| 1692951 | UCUGAAGAUGAAGAGUUUAGA | yyyyyyfyfffyyyyyyyyyy | ssooooooooooooooooss | 984 |
| 1692953 | AAGAGAAAAUAUAUUUACUAA | yyyyyyfyfffyyyyyyyyyy | ssooooooooooooooooss | 985 |

In Table 11 and Table 12, the structure for the 6-palmitamidohexyl phosphoryl conjugate group attached to the 5′-OH of the oligonucleotide is:

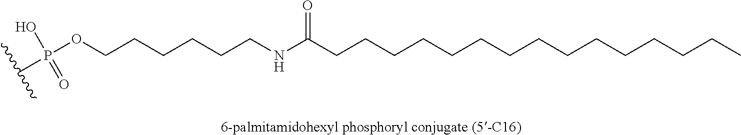 [SMILES: *P(=O)(O)OCCCCCCNC(=O)CCCCCCCCCCCCCCC]

TABLE 11
5′ 6-palmitamidohexyl phosphoryl conjugated sense oligonucleotides

| | | | Sense Strand | SEQ |
|---|---|---|---|---|
| Compound | Sense Oligo. Sequence | Sense Strand Sugar Motif | Internucleoside Linkage | ID |
| No. | (5′ to 3′) | (5′ to 3′) | (5′ to 3′) | NO |
| 1717680 | CAAUUUCUGUCUCAUCUUAAA | yyyyyyfyfffyyyyyyyyyy | ssooooooooooooooooss | 986 |
| 1731395 | CAAUUUCUGUCUCAUCUUAAA | yyyyyyyyyyyyyyyyyyyyy | ssooooooooooooooooss | 987 |
| 1731393 | CAAUUUCUGUCUCAUCUUAAA | yyyyyyyyyffyyyyyyyyyy | ssooooooooooooooooss | 988 |
| 1731392 | CAAUUUCUGUCUCAUCUUAAA | yyyyyyfyfyfyyyyyyyyyy | ssooooooooooooooooss | 989 |
| 1731394 | CAAUUUCUGUCUCAUCUUAAA | yyyyyyyyyffyyyyyyyyyy | ssoooooozozoooooooss | 990 |
| 1731396 | CAAUUUCUGUCUCAUCUUAAA | yyyyyyyyyydyyyyyyyyyy | ssooooooooooooooooss | 991 |
| 1736406 | CAAUUUCUGUCUCAUCUUAAA | eeyyyyfyfffyyyyyyyyee | ssooooooooooooooooss | 992 |
| 1736407 | CAAUUUCUGUCUCAUCUUAAA | eeyyyyyyyffyyyyyyyyee | ssooooooooooooooooss | 993 |
| 1738167 | CAAUUUCUGUCUCAUCUUAAA | eeyyyyfyfyfyyyyyyyyee | ssooooooooooooooooss | 994 |
| 1738168 | CAAUUUCUGUCUCAUCUUAAA | eeyyyyyyyffyyyyyyyyee | ssooooooooooooooooss | 995 |
| 1738169 | CAAUUUCUGUCUCAUCUUAAA | yyyyyyyyyffyyyyyyyyyy | ssooooooooooooooooss | 996 |
| 1736490 | CAAUUUCUGUCUCAUCUUAAA | eeyyyyfyfffyyyyyyyyee | ssooooooooooooooooss | 997 |
| 1736405 | AUUUCUGUCUCAUCUUAAA | yyyyyyfyfffyyyyyyyy | ssooooooooooooooooss | 998 |
| 1737448 | AUUUCUGUCUCAUCUUAAA | yyyyfyfffyyyyyyyyyy | ssooooooooooooooooss | 999 |
| 1738166 | AUUUCUGUCUCAUCUUAAA | yyyyyyyffyyyyyyyyyy | ssooooooooooooooooss | 1000 |

TABLE 12
5′ 6-palmitamidohexyl phosphoryl conjugated sense oligonucleotides

| | | | Sense Strand | |
|---|---|---|---|---|
| Compound | | Sense Strand Sugar | Internucleoside | SEQ |
| No. | Sense Oligo. Sequence (5′ to 3′) | Motif (5′ to 3′) | Linkage (5′ to 3′) | ID NO |
| 1750677 | CAAUUUCUGUCUCAUCUUAAA | eeyyyyyyyffyyyyyyyyee | ssooooooooooooooooss | 1256 |
| 1753984 | AAGCUACAGAAUCUAUUUATA | eeyyyyyyyffyyyyyyyyee | ssooooooooooooooooss | 1257 |

In Table 13, the structure for the 6-aminohexyl phosphoryl conjugate group attached to the 5′-OH of the oligonucleotide is:

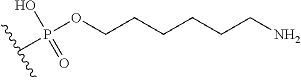 [SMILES: *P(=O)(O)OCCCCCCN]

TABLE 13
5′ 6-aminohexyl phosphoryl conjugated sense oligonucleotides

| | | | Sense Strand | |
|---|---|---|---|---|
| Compound | | Sense Strand Sugar | Internucleoside | SEQ |
| No. | Sense Oligo. Sequence (5′ to 3′) | Motif (5′ to 3′) | Linkage (5′ to 3′) | ID NO |
| 1738338 | CAAUUUCUGUCUCAUCUUAAA | eeyyyyfyfyfyyyyyyyyee | ssooooooooooooooooss | 1289 |
| 1753982 | AAGCUACAGAAUCUAUUUAUA | eeyyyyfyfyfyyyyyyyyee | ssooooooooooooooooss | 1288 |
| 1750001 | AAGCUACAGAAUCUAUUUATA | eeyyyyyyyffyyyyyyyyee | ssooooooooooooooooss | 1290 |

In Table 14 a bicycle ligand, BCY17901, is conjugated to the 3′ end of the sense oligonucleotide via click reaction to a 6-(BCN-carbamate)-2-(hydroxymethyl)hexyl phosphoryl linker. BCY17901 contains the amino acid sequence CP[HyP]DAYLGC[tBuGly]SYCEPW[K(N₃)] (SEQ ID NO: 1253), wherein [HyP]represents trans-4-hydroxy-L-proline, [tBuGly] represents t-butyl-glycine, and [K(N₃)] represents 6-azido lysine. BCY17901 has an N-terminal acetyl modification and a C-terminal amide modification, and each cysteine forms a covalent bond with the molecular scaffold 1,1′,1″-(1,3,5-triazinane-1,3,5-triyl)tris(2-bromoethanone) (TATB). The structure for the 3′-BCY17901 conjugate group is:

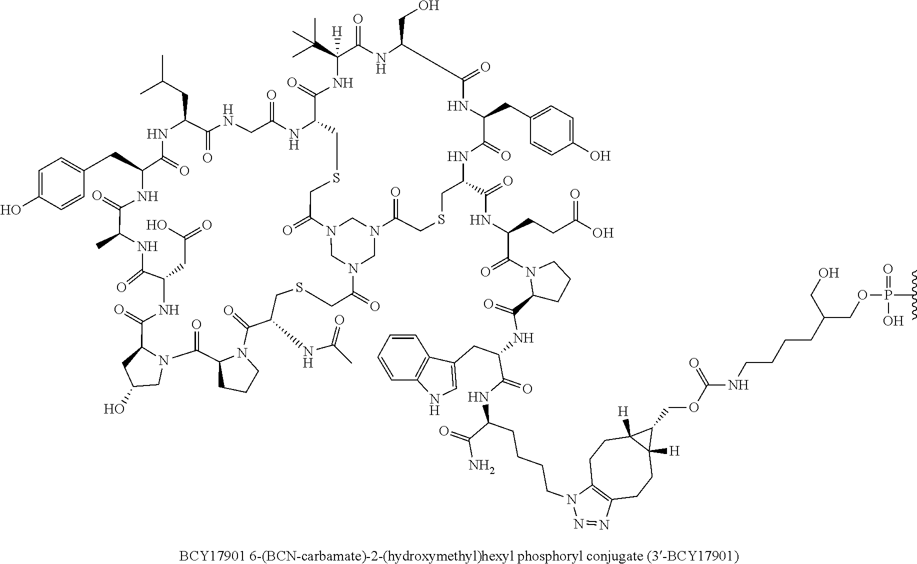 [SMILES: *P(=O)(O)OCC(CO)CCCCNC(=O)OC[C@@H]1[C@@H]2CCc3nnn(CCCC[C@H](NC(=O)[C@H](Cc4c[nH]c5ccccc45)NC(=O)[C@@H]4CCCN4C(=O)[C@H](CCC(=O)O)NC(=O)[C@@H]4CSCC(=O)N5CN6CN(C5)C(=O)CSC[C@H](NC(C)=O)C(=O)N5CCC[C@H]5C(=O)N5C[C@H](O)C[C@H]5C(=O)N[C@@H](CC(=O)O)C(=O)N[C@@H](C)C(=O)N[C@@H](Cc5ccc(O)cc5)C(=O)N[C@@H](CC(C)C)C(=O)NCC(=O)N[C@@H](CSCC6=O)C(=O)N[C@@H](C(C)(C)C)C(=O)N[C@@H](CO)C(=O)N[C@@H](Cc5ccc(O)cc5)C(=O)N4)C(N)=O)c3CC[C@@H]21]

TABLE 14
Sense oligonucleotides conjugated to BCY17901 at the 3′-end

| | | | Sense Strand | SEQ |
|---|---|---|---|---|
| Compound | Sense Oligo. Sequence | Sense Strand Sugar Motif | Internucleoside Linkage | ID |
| No. | (5′ to 3′) | (5′ to 3′) | (5′ to 3′) | NO |
| 1696305 | CAAUUUCUGUCUCAUCUUAAA | yyyyyyfyfffyyyyyyyyyy | ssooooooooooooooooss | 1001 |
| 1722764 | AAGCUACAGAAUCUAUUUAUA | yyyyyyfyfffyyyyyyyyyy | ssooooooooooooooooss | 1002 |
| 1722765 | UAAGUAAUUUUUUCAAAGAAA | yyyyyyfyfffyyyyyyyyyy | ssooooooooooooooooss | 1003 |
| 1722767 | AAGAUUAAGACUAAAACUUAA | yyyyyyfyfffyyyyyyyyyy | ssooooooooooooooooss | 1004 |
| 1722768 | CUGUUGGAUCUUGUAAACAUA | yyyyyyfyfffyyyyyyyyyy | ssooooooooooooooooss | 1005 |
| 1722770 | CAAAAUAAGUGUAUAAAAUGA | yyyyyyfyfffyyyyyyyyyy | ssooooooooooooooooss | 1006 |
| 1722771 | AAGAGUUUAGUUUUAAAACUA | yyyyyyfyfffyyyyyyyyyy | ssooooooooooooooooss | 1007 |
| 1722773 | UCUGAAGAUGAAGAGUUUAGA | yyyyyyfyfffyyyyyyyyyy | ssooooooooooooooooss | 1008 |
| 1722774 | AAGAGAAAAUAUAUUUACUAA | yyyyyyfyfffyyyyyyyyyy | ssooooooooooooooooss | 1009 |

In Table 15 a bicycle ligand, BCY17901, is conjugated at the 5′-end of the sense oligonucleotide via click reaction to a 6-(BCN-carbamate)hexyl phosphoryl linker. BCY17901 contains the amino acid sequence CP[HyP]DAYLGC[tBuGly]SYCEPW[K(N₃)] (SEQ ID NO: 1253), wherein [HyP] represents trans-4-hydroxy-L-proline, [tBuGly] represents t-butyl-glycine, and [K(N₃)] represents 6-azido lysine. BCY17901 has an N-terminal acetyl modification and a C-terminal amide modification, and each cysteine forms a covalent bond with the molecular scaffold 1,1′,1″-(1,3,5-triazinane-1,3,5-triyl)tris(2-bromoethanone) (TATB). The structure for the 5′-BCY17901 conjugate group is:

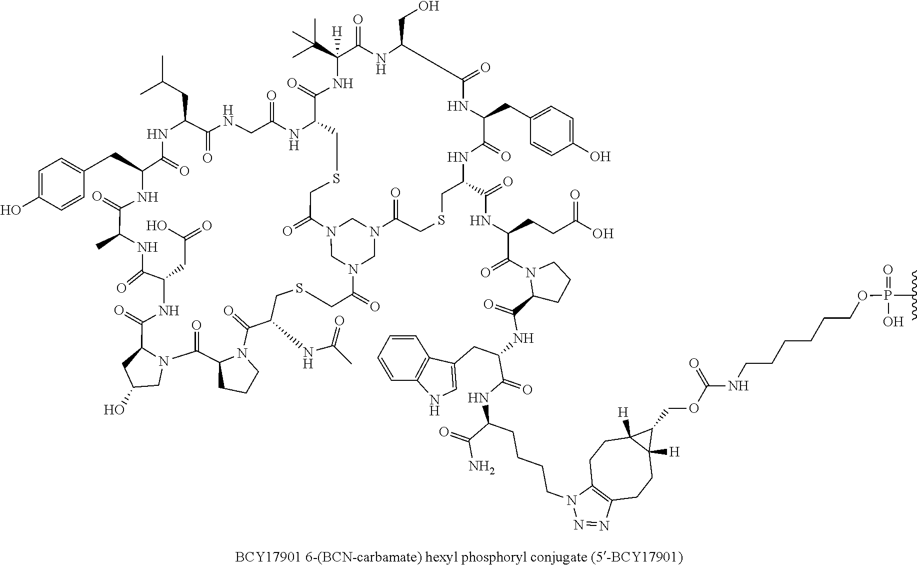 [SMILES: *P(=O)(O)OCCCCCCNC(=O)OC[C@@H]1[C@@H]2CCc3nnn(CCCC[C@H](NC(=O)[C@H](Cc4c[nH]c5ccccc45)NC(=O)[C@@H]4CCCN4C(=O)[C@H](CCC(=O)O)NC(=O)[C@@H]4CSCC(=O)N5CN6CN(C5)C(=O)CSC[C@H](NC(C)=O)C(=O)N5CCC[C@H]5C(=O)N5C[C@H](O)C[C@H]5C(=O)N[C@@H](CC(=O)O)C(=O)N[C@@H](C)C(=O)N[C@@H](Cc5ccc(O)cc5)C(=O)N[C@@H](CC(C)C)C(=O)NCC(=O)N[C@@H](CSCC6=O)C(=O)N[C@@H](C(C)(C)C)C(=O)N[C@@H](CO)C(=O)N[C@@H](Cc5ccc(O)cc5)C(=O)N4)C(N)=O)c3CC[C@@H]21]

TABLE 15
Sense oligonucleotides conjugated to BCY17901 at the 5′-end

| | | | Sense Strand | SEQ |
|---|---|---|---|---|
| Compound | Sense Oligo. Sequence | Sense Strand Sugar Motif | Internucleoside Linkage | ID |
| No. | (5′ to 3′) | (5′ to 3′) | (5′ to 3′) | NO |
| 1719928 | CAAUUUCUGUCUCAUCUUAAA | yyyyyyfyfffyyyyyyyyyy | ssooooooooooooooooss | 1010 |
| 1757454 | CAAUUUCUGUCUCAUCUUAAA | eeyyyyyyyffyyyyyyyyee | ssooooooooooooooooss | 1011 |
| 1753740 | CAAUUUCUGUCUCAUCUUAAA | eeyyyyfyfyfyyyyyyyyee | ssooooooooooooooooss | 1012 |
| 1757456 | AAGCUACAGAAUCUAUUUATA | eeyyyyyyyffyyyyyyyyee | ssooooooooooooooooss | 1013 |
| 1757458 | AAGCUACAGAAUCUAUUUAUA | eeyyyyfyfyfyyyyyyyyee | ssooooooooooooooooss | 1014 |
| 1757460 | AAGAUUAAGACUAAAACUUAA | eeyyyyyyyffyyyyyyyyee | ssooooooooooooooooss | 1015 |
| 1757462 | AAGAUUAAGACUAAAACUUAA | eeyyyyfyfyfyyyyyyyyee | ssooooooooooooooooss | 1016 |

Oligomeric duplexes conjugated to BCY17901 at the end of a sense modified oligonucleotide were prepared following the scheme and general procedures. BCY17901 was synthesized as described in International Patent Application Publication No. WO2022/101633, and International Patent Application Publication No. WO2023/056388.

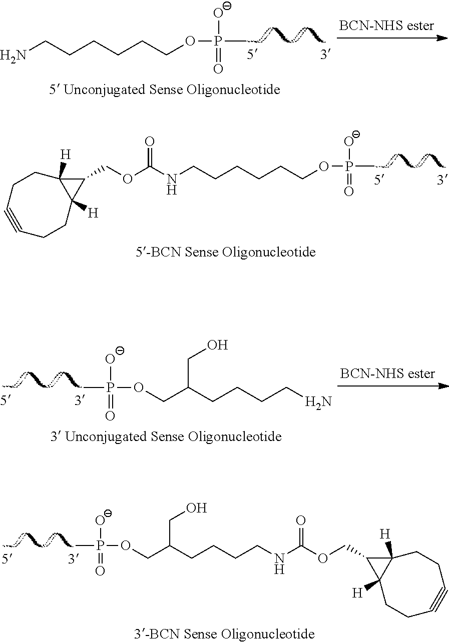

Sense oligonucleotide with a 6-aminohexyl linker attached via a phosphoryl group at the 5′ end were synthesized following standard procedure. 25-30 mg, 2.5-3.0 gmol, 1 equiv. sense oligonucleotide was dissolved in 300 μL of 0.1 M borax buffer pH 8.5. BCN—NHS ester (3 equiv.) dissolved in DMF (300 μL) was added to the sense oligonucleotide solution, and the reaction mixture stirred at room temperature. Reaction completion was confirmed after 30-60 min by LCMS. Crude reaction mixtures were purified by strong anion exchange (SAX) HPLC, using a gradient of mobile phase A (100 mM NH₄OAc in 3:7 ACN:H₂O) and mobile phase B (100 mM NH₄OAc, 1.5 M NaBr in 3:7 ACN:H₂O). After purification, oligonucleotides were desalted by RP HPLC on a 5 g Sep-Pak C18 cartridge, loaded and washed with 1.0 M NaCl, then eluted in 1:1 ACN:H₂O. Desalted fractions were lyophilized to yield BCN-conjugated sense oligonucleotide.

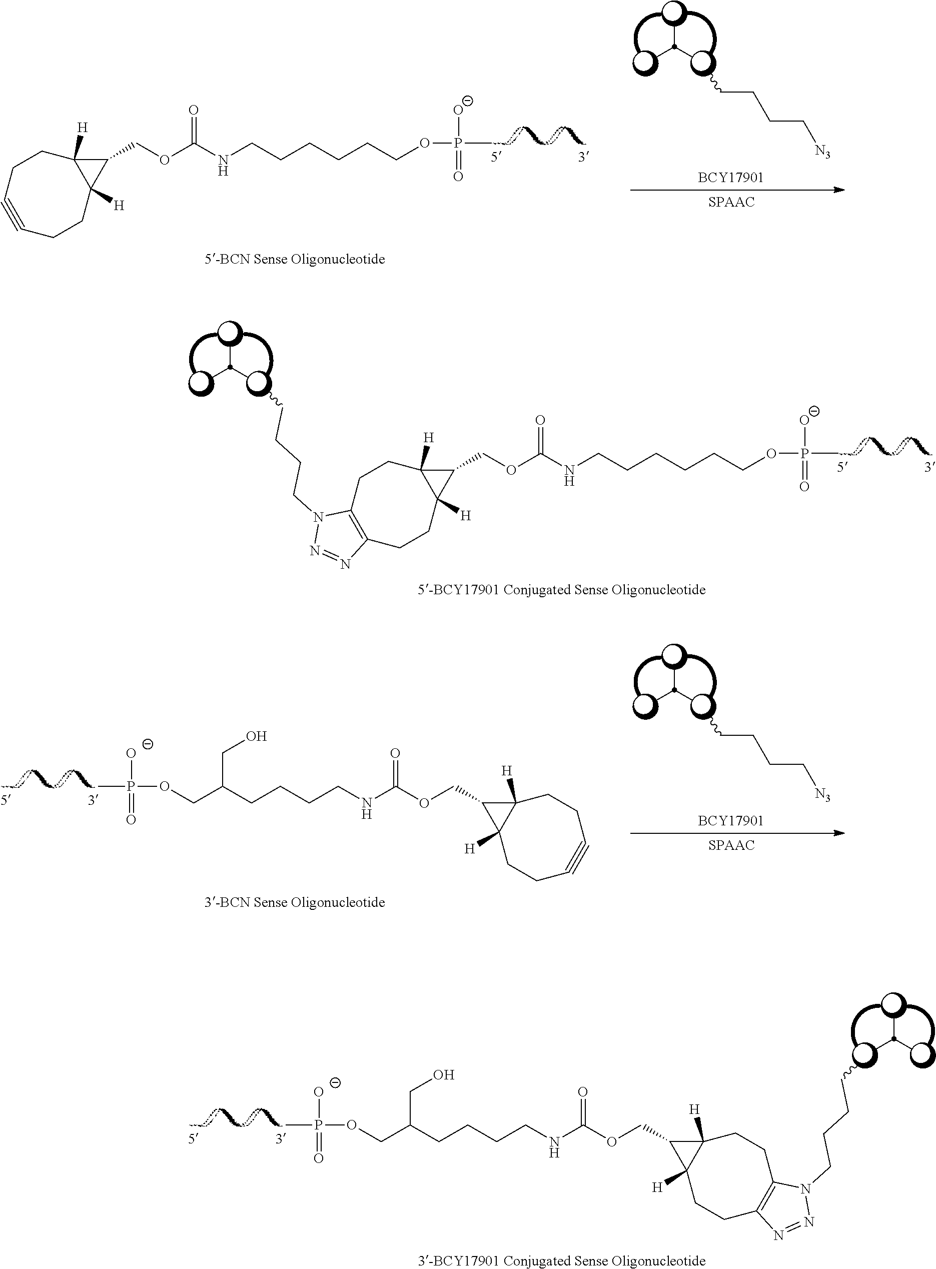

BCY17901 was conjugated to BCN-sense oligonucleotide using a strain-promoted azide-alkyne click (SPAAC) reaction. BCY17901 (1.5 equiv.) dissolved in DMF (300 μL) was added to a solution of BCN-sense oligonucleotide (˜20 mg, 2.5-3.0 gmol, 1 equiv.) dissolved in H₂O (1 mL), stirred at room temperature overnight, and conjugation was confirmed by LCMS. Crude reaction mixtures were purified by SAX HPLC, using a gradient of mobile phase A and mobile phase B (described above). After purification, oligonucleotides were desalted by RP HPLC on a 5 g Sep-Pak C18 cartridge. Desalted fractions were lyophilized to yield 5′ BCY17901-conjugated sense oligonucleotide.

For synthesis of 3′ BCY-conjugated sense oligonucleotide, procedure described above was followed, with the following modification: unconjugated sense oligonucleotide starting material has a 2-(hydroxymethyl)-6-aminohexyl phosphoryl group conjugated to the 3′-OH.

Solutions of individual sense and antisense oligonucleotides were prepared at 2× concentration in Dulbecco's Phosphate Buffered Saline (DPBS, no calcium, no magnesium) and filtered through 0.22 m filters. The 5′-BCY17901 conjugated sense oligonucleotide and antisense oligonucleotide were mixed 1:1 (v:v) and incubated at room temperature for 1-2 hours to give the final 1× concentration of oligomeric duplex in DPBS. Duplex formation was verified by size-exclusion chromatography (SEC) and masses verified by HPLC-MS. Annealed 5′-BCY17901 conjugated modified oligomeric duplexes were stored at 4° C.

### Design of Oligomeric Duplexes

Oligomeric duplex compounds prepared with antisense oligonucleotide compound numbers and corresponding sense oligonucleotide compound numbers are listed in Table 16.

TABLE 16
Oligomeric duplexes targeted to human PLN

| Compound | Antisense | Sense |
|---|---|---|
| Number | Compound No. | Compound No. |
| 1700644 | 1652826 | 1696305 |
| 1722807 | 1652826 | 1719928 |
| 1726638 | 1652709 | 1722764 |
| 1726639 | 1691449 | 1722765 |
| 1726640 | 1691837 | 1722767 |
| 1726641 | 1692941 | 1722768 |
| 1726645 | 1692943 | 1722770 |
| 1726646 | 1692945 | 1722771 |
| 1726647 | 1692950 | 1722773 |
| 1726648 | 1692952 | 1722774 |
| 1669056 | 1652709 | 1652825 |
| 1669057 | 1652826 | 1652841 |
| 1703266 | 1691449 | 1691836 |
| 1703330 | 1691837 | 1692947 |
| 1703395 | 1692941 | 1692948 |
| 1703527 | 1692943 | 1692949 |
| 1703530 | 1692945 | 1692946 |
| 1703608 | 1692950 | 1692951 |
| 1703668 | 1692952 | 1692953 |
| 1722804 | 1652826 | 1717680 |
| 1735533 | 1731386 | 1717680 |
| 1735536 | 1731388 | 1717680 |
| 1735539 | 1731387 | 1717680 |
| 1735542 | 1731389 | 1717680 |
| 1735545 | 1731390 | 1717680 |
| 1735551 | 1731397 | 1717680 |
| 1735554 | 1652826 | 1731395 |
| 1735557 | 1652826 | 1731393 |
| 1735560 | 1652826 | 1731392 |
| 1735563 | 1652826 | 1731394 |
| 1735566 | 1652826 | 1731396 |
| 1735569 | 1731387 | 1731393 |
| 1735572 | 1731388 | 1731393 |
| 1735575 | 1731389 | 1731393 |
| 1735578 | 1731390 | 1731393 |
| 1735581 | 1731387 | 1731392 |
| 1735584 | 1731387 | 1731394 |
| 1735587 | 1731387 | 1731396 |
| 1735590 | 1731387 | 1731395 |
| 1739642 | 1736400 | 1717680 |
| 1739646 | 1736401 | 1736405 |
| 1739649 | 1736401 | 1737448 |
| 1739652 | 1736401 | 1738166 |
| 1739655 | 1736402 | 1717680 |
| 1739658 | 1736403 | 1717680 |
| 1739661 | 1736404 | 1717680 |
| 1739664 | 1738170 | 1717680 |
| 1739667 | 1738171 | 1717680 |
| 1739670 | 1738172 | 1717680 |
| 1739673 | 1738173 | 1717680 |
| 1739676 | 1738174 | 1717680 |
| 1739679 | 1652826 | 1736406 |
| 1739682 | 1652826 | 1736407 |
| 1739685 | 1652826 | 1738167 |
| 1739688 | 1652826 | 1738168 |
| 1739691 | 1652826 | 1738169 |
| 1739694 | 1736404 | 1736407 |
| 1739697 | 1738172 | 1736407 |
| 1739700 | 1738171 | 1736407 |
| 1750177 | 1736400 | 1736406 |
| 1750180 | 1749976 | 1717680 |
| 1750207 | 1749977 | 1717680 |
| 1750213 | 1744533 | 1717680 |
| 1750219 | 1744534 | 1717680 |
| 1750225 | 1746643 | 1717680 |
| 1750231 | 1746644 | 1717680 |
| 1750237 | 1746645 | 1717680 |
| 1750243 | 1749974 | 1717680 |
| 1750248 | 1749975 | 1717680 |
| 1750254 | 1749974 | 1736407 |
| 1750259 | 1749974 | 1738168 |
| 1750268 | 1749974 | 1738167 |
| 1750274 | 1746643 | 1736407 |
| 1750280 | 1746643 | 1736490 |
| 1750292 | 1746644 | 1736407 |
| 1750301 | 1736402 | 1736407 |
| 1750355 | 1652826 | 1736490 |
| 1750676 | 1749977 | 1738167 |
| 1750683 | 1738170 | 1736407 |
| 1755748 | 1746643 | 1750677 |
| 1755751 | 1746644 | 1738167 |
| 1755757 | 1753975 | 1753984 |
| 1755763 | 1753981 | 1753984 |
| 1757465 | 1746643 | 1757454 |
| 1757468 | 1746644 | 1753740 |
| 1757471 | 1753975 | 1757456 |
| 1757474 | 1753976 | 1757458 |
| 1757477 | 1753977 | 1757460 |
| 1757480 | 1753978 | 1757462 |
| 1779744 | 1753981 | 1757456 |
| 1758638 | 1753976 | 1753982 |
| 1809053 | 1801600 | 1757458 |
| 1809061 | 1801601 | 1757458 |
| 1809071 | 1805817 | 1753982 |
| 1809072 | 1801601 | 1753982 |
| 1809073 | 1801600 | 1753982 |
| 1809074 | 1801602 | 1753982 |
| 1758632 | 1746644 | 1738338 |
| 1779726 | 1753981 | 1750001 |

### Example 2: Design of Modified Oligonucleotides Targeted to Human PLN

Modified oligonucleotides complementary to a human PLN RNA were designed as described in Tables 17-18. “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each antisense oligonucleotide is 100% complementary to SEQ ID NO: 2 (GENBANK Accession No. (NC_000006.12 truncated from nucleosides 118545001 to 118565000).

The modified oligonucleotides in Tables 17-18 are 16 nucleosides in length, wherein the sugar motifs for the modified oligonucleotides are as designated in the column labeled “Sugar Motif (5′ to 3′)”; wherein each ‘e’ represents a 2′-MOE sugar moiety, each ‘k’ represents a cEt sugar moiety, and each ‘d’ represents a 2′-deoxy sugar moiety. The internucleoside linkage motifs for the modified oligonucleotides are presented in the column labeled “Internucleoside Linkage (5′ to 3′)”, wherein each ‘s’ represents a phosphorothioate internucleoside linkage, and each ‘o’ represents a phosphodiester internucleoside linkage. Each cytosine residue is a 5-methylcytosine.

Each modified oligonucleotide in Table 17 is conjugated to a 6-palmitamidohexyl phosphoryl conjugate group attached to the 5′-OH of the oligonucleotide. The structure for the conjugate group 5′-C16 is depicted in Example 1.

TABLE 17
5′ 6-palmitamidohexyl conjugated modified oligonucleotides targeted to human PLN

| | SEQ | SEQ | | | | |
|---|---|---|---|---|---|---|
| | ID No: | ID No: | | | Internucleoside | SEQ |
| Compound | 2 Start | 2 Stop | Sequence | Sugar Motif | Linkage | ID |
| No. | Site | Site | (5′ to 3′) | (5′ to 3′) | (5′ to 3′) | NO |
| 1558169 | 5499 | 5514 | ACGAGTATATTAGG | kkdddddddddkekek | sosssssssssooos | 1017 |
| | | | AA | | | |

Each modified oligonucleotide in Table 18 is conjugated at the 5′-end to a bicycle ligand, BCY17901, via click reaction to a 6-(BCN-carbamate)hexyl phosphoryl linker. BCY17901 contains the amino acid sequence CP[HyP]DAYLGC[tBuGly]SYCEPW[K(N₃)] (SEQ ID NO: 1253), wherein [HyP]represents trans-4-hydroxy-L-proline, [tBuGly] represents t-butyl-glycine, and [K(N₃)] represents 6-azido lysine. BCY17901 has an N-terminal acetyl modification and a C-terminal amide modification, and each cysteine forms a covalent bond with the molecular scaffold 1,1′,1″-(1,3,5-triazinane-1,3,5-triyl)tris(2-bromoethanone) (TATB). The structure for the BCY17901 conjugate group, 5′-BCY17901, is depicted in Example 1.

TABLE 18
BCY17901 conjugated modified oligonucleotides targeted to human PLN

| | SEQ | SEQ | | | | |
|---|---|---|---|---|---|---|
| | ID | ID | | | | |
| | No: 2 | No: 2 | | | Internucleoside | SEQ |
| Compound | Start | Stop | Sequence | Sugar Motif | Linkage | ID |
| No. | Site | Site | (5′ to 3′) | (5′ to 3′) | (5′ to 3′) | NO |
| 1696302 | 5499 | 5514 | ACGAGTATATTAGGAA | kkdddddddddkekek | sosssssssssooos | 1018 |

### Example 3: Design of Modified Oligonucleotides Targeted to Mouse PLN RNA

Modified oligonucleotides complementary to a mouse PLN RNA were designed as described in Tables 19-21. “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each antisense oligonucleotide listed is 100% complementary to SEQ ID NO: 1052 (GENBANK Accession No. (NC_000076.6 truncated from nucleosides 53335001 to 53349000).

The modified oligonucleotides in Tables 19-21 are 16 nucleosides in length, wherein the sugar motifs for the modified oligonucleotide are as designated in the column labeled “Sugar Motif (5′ to 3′)”; wherein each ‘e’ represents a 2′-MOE sugar moiety, each ‘k’ represents a cEt sugar moiety, and each ‘d’ represents a 2′-deoxy sugar moiety. The internucleoside linkage motifs for the modified oligonucleotide are presented in the column labeled “Internucleoside Linkage (5′ to 3′)”, wherein each ‘s’ represents a phosphorothioate internucleoside linkage, and each ‘o’ represents a phosphodiester internucleoside linkage. Each cytosine residue is a 5-methylcytosine.

TABLE 19
Modified oligonucleotide targeted to mouse PLN

| | SEQ ID | SEQ ID | | | | |
|---|---|---|---|---|---|---|
| | No: | No: | | | Internucleoside | SEQ |
| Compound | 1052Start | 1052Stop | Sequence | Sugar Motif | Linkage | ID |
| No. | Site | Site | (5′ to 3′) | (5′ to 3′) | (5′ to 3′) | NO |
| 1639304 | 3812 | 3827 | TGAGAATTGTAGAGGT | kkkdddddddddkkke | sssssssssssssss | 1019 |

Each modified oligonucleotide in Table 20 is conjugated to a 6-palmitamidohexyl phosphoryl conjugate group attached to the 5′-OH of the oligonucleotide. The structure for the conjugate group 5′-C16 is depicted in Example 1.

TABLE 20
5′ 6-palmitamidohexyl conjugated modified oligonucleotides targeted to mouse PLN

| | SEQ | SEQ | | | | |
|---|---|---|---|---|---|---|
| | ID | ID | | | | |
| | No: | No: | | | | |
| | 1052 | 1052 | | | Internucleoside | SEQ |
| Compound | Start | Stop | Sequence | Sugar Motif | Linkage | ID |
| No. | Site | Site | (5′ to 3′) | (5′ to 3′) | (5′ to 3′) | NO |
| 1604918 | 3812 | 3827 | TGAGAATTGTAGAGGT | kkkdddddddddkkke | sssssssssssssss | 1020 |
| 1653557 | 3812 | 3827 | TGAGAATTGTAGAGGT | kkkdddddddddkkke | ooosssssssssoos | 1021 |

Each modified oligonucleotide in Table 21 is conjugated at the 5′-end to a bicycle ligand, BCY17901, via click reaction to a 6-(BCN-carbamate)hexyl phosphoryl linker. BCY17901 contains the amino acid sequence CP[HyP]DAYLGC[tBuGly]SYCEPW[K(N₃)], wherein [HyP] represents trans-4-hydroxy-L-proline, [tBuGly] represents t-butyl-glycine, and [K(N₃)] represents 6-azido lysine. BCY17901 has an N-terminal acetyl modification and a C-terminal amide modification, and each cysteine forms a covalent bond with the molecular scaffold 1,1′,1″-(1,3,5-triazinane-1,3,5-triyl)tris(2-bromoethanone) (TATB). The structure for the BCY17901 conjugate group, 5′-BCY17901, is depicted in Example 1.

TABLE 21
BCY17901 conjugated modified oligonucleotides targeted to mouse PLN

| | SEQ | SEQ | | | | |
|---|---|---|---|---|---|---|
| | ID | ID | | | | |
| | No: | No: | | | | |
| | 1052 | 1052 | | | Internucleoside | SEQ |
| Compound | Start | Stop | Sequence | Sugar Motif | Linkage | ID |
| No. | Site | Site | (5′ to 3′) | (5′ to 3′) | (5′ to 3′) | NO |
| 1683006 | 3812 | 3827 | TGAGAATTGTAGAGGT | kkkdddddddddkkke | sssssssssssssss | 1022 |
| 1683007 | 3812 | 3827 | TGAGAATTGTAGAGGT | kkkdddddddddkkke | ooosssssssssoos | 1023 |

### Example 4: Potency of 5′-C16 or 5′-BCY17901 Conjugated Oligonucleotides Targeting Mouse PLN

hTfR^{+/-} knock-in mice were used to determine effects of modified oligonucleotides on mouse PLN. hTfR^{+/-} knock-in mice were divided into groups of 3 mice each. Each mouse received intravenous injections of modified oligonucleotide once a week for three weeks for a total of 3 treatments (on Days 1, 6, and 13) at various doses. One group of 4 mice received intravenous injections of PBS once a week for three weeks (a total of 3 treatments). One group of 3 mice remained untreated (Naïve). PBS-injected group and non-treated naïve group served as control groups to which oligonucleotide-treated groups were compared.

Eight days post final treatment (Day 21), mice were sacrificed and RNA was extracted from mouse heart for real-time RTPCR analysis of PLN RNA expression. Mouse PLN primer probe set RTS37645 (forward sequence TCTGCTCTCTCTAGGGTTTGA, designated herein as SEQ ID NO: 1053; reverse sequence GGAGATACATGAGAGCGACAAG, designated herein as SEQ ID NO: 1054; probe sequence ACAGCCAACACAGCAAGATGTTCC, designated herein as SEQ ID NO: 1055) was used to measure mouse PLN RNA levels. PLN RNA levels were normalized to total RNA content, as measured by RIBOGREEN®. Results are presented as percent PLN RNA, relative to amount of PLN in a combination of the PBS treated animals and non-treated naïve animals (% control). Half maximal effective dose (ED₅₀) of each modified oligonucleotide was calculated using GraphPad Prism 7 software (GraphPad Software, San Diego, CA).

TABLE 22
Reduction of mouse PLN RNA

| Compound | | Dose | PLN RNA (% control) | |
|---|---|---|---|---|
| No. | Conjugate | (mg/kg) | Heart | ED50 (mg/kg) |
| PBS + Naïve | — | — | 100 | — |
| 1604918 | 5′-C16 | 5.7 | 54 | 6.44 |
| | | 17.2 | 23 | |
| | | 51.7 | 7 | |
| 1639304 | None | 16.7 | 85 | 74.65 |
| | | 50.1 | 58 | |
| | | 150.3 | 34 | |
| 1653557 | 5′-C16 | 5.7 | 57 | 6.94 |
| | | 17.2 | 20 | |
| | | 51.7 | 4 | |
| 1683006 | 5′-BCY17901 | 5.8 | 60 | 33.05 |
| | | 17.5 | 64 | |
| | | 52.5 | 42 | |
| 1683007 | 5′-BCY17901 | 5.8 | 52 | 8.81 |
| | | 17.5 | 46 | |
| | | 52.5 | 36 | |

As shown in Table 22, modified oligonucleotide was more potent in decreasing the level of PLN RNA in hearts of transgenic mice when conjugated to a 6-palmitamidohexyl phosphoryl group (Compound Nos 1604918 and 1653557) or a human transferrin receptor 1-binding peptide, BCY17901 (Compound Nos 1683006 and 1683007) as compared to potency of unconjugated oligonucleotide (Compound No 1639304).

### Example 5: Activity of Conjugated Oligomeric Duplexes Targeting Human PLN

hPLN^{tg/+} heterozygous mice were used to determine effects of 3′-C16-conjugated oligomeric duplexes on human PLN. hTfR^{tg/+}/hPLN^{tg/tg} knock-in mice, generated by crossing hTfR^{tg/+} and hPLN^{tg/tg}, were used to determine effects of 3′-BCY17901-conjugated oligomeric duplexes on human PLN. The antisense strand sugar motif (5′ to 3′) for all compounds in Table 23 is efyyyfyyyyyyyfyfyyyyyyy and the sense strand sugar motif (5′ to 3′) for all compounds in Table 23 is yyyyyyfyfffyyyyyyyyyy. Transgenic mice (hPLN^{tg/+} or hTfR^{tg/+}/hPLN^{tg/tg} knock-in) were divided into groups of 3-4 mice each. Each mouse received a single subcutaneous injection of oligomeric duplex. C16-conjugated oligomeric duplexes were administered at a dose of 50 mg/kg; and BCY17901-conjugated oligomeric duplexes were administered at a dose of 5 mg/kg. One group of 3-4 mice (for each strain) received a single subcutaneous injection of PBS. PBS-injected group served as the control group to which duplex-treated groups were compared. Seven (7) days post final treatment, mice were sacrificed and RNA extracted from mouse heart for real-time RTPCR analysis of PLN RNA expression. Human PLN primer probe set ABI53044 (forward sequence CACCCGTAAGACTTCATACAACACA, designated herein as SEQ ID NO: 1056; reverse sequence TGGCAGCCAAATATGAGATAACTGT, designated herein as SEQ ID NO: 1057; probe sequence TGCCAAGGCTACCTAA, designated herein as SEQ ID NO: 1058) was used to measure human PLN RNA levels. PLN RNA levels were normalized to total RNA content, as measured by RIBOGREEN®. Results are presented as percent human PLN RNA, relative to amount of human PLN RNA in corresponding PBS treated animals (% control). n/d refers to datapoints that were not determined.

TABLE 23
Reduction of human PLN RNA

| | | Nucleobase Sequence of | SEQ ID NO: | Single | PLN RNA |
|---|---|---|---|---|---|
| Compound | | Antisense Strand (5′ to 3′) | Antisense and | Dose | (% control) |
| No. | Conjugate | and Sense Strand (5′ to 3′) | Sense | (mg/kg) | Heart |
| PBS | | | | | 100 |
| 1669057 | 3′-C16 | TUUAAGAUGAGACAGAAAUUGAU | 939 | 50 | 38 |
| | | CAAUUUCUGUCUCAUCUUAAA | 978 | | |
| 1700644 | BCY17901 | TUUAAGAUGAGACAGAAAUUGAU | 939 | 5 | 64 |
| | | CAAUUUCUGUCUCAUCUUAAA | 1001 | | |
| 1703266 | 3′-C16 | TUUCUUUGAAAAAAUUACUUAAA | 941 | 50 | 91 |
| | | UAAGUAAUUUUUUCAAAGAAA | 979 | | |
| 1726639 | BCY17901 | TUUCUUUGAAAAAAUUACUUAAA | 941 | 5 | 89 |
| | | UAAGUAAUUUUUUCAAAGAAA | 1003 | | |
| 1703330 | 3′-C16 | TUAAGUUUUAGUCUUAAUCUUGA | 942 | 50 | 48 |
| | | AAGAUUAAGACUAAAACUUAA | 980 | | |
| 1726640 | BCY17901 | TUAAGUUUUAGUCUUAAUCUUGA | 942 | 5 | 37 |
| | | AAGAUUAAGACUAAAACUUAA | 1004 | | |
| 1703395 | 3′-C16 | TAUGUUUACAAGAUCCAACAGAU | 943 | 50 | 48 |
| | | CUGUUGGAUCUUGUAAACAUA | 981 | | |
| 1726641 | BCY17901 | TAUGUUUACAAGAUCCAACAGAU | 943 | 5 | 47 |
| | | CUGUUGGAUCUUGUAAACAUA | 1005 | | |
| 1703527 | 3′-C16 | TCAUUUUAUACACUUAUUUUGAA | 944 | 50 | 52 |
| | | CAAAAUAAGUGUAUAAAAUGA | 982 | | |
| 1726645 | BCY17901 | TCAUUUUAUACACUUAUUUUGAA | 944 | 5 | 46 |
| | | CAAAAUAAGUGUAUAAAAUGA | 1006 | | |
| 1703530 | 3′-C16 | TAGUUUUAAAACUAAACUCUUCA | 945 | 50 | 89 |
| | | AAGAGUUUAGUUUUAAAACUA | 983 | | |
| 1726646 | BCY17901 | TAGUUUUAAAACUAAACUCUUCA | 945 | 5 | 85 |
| | | AAGAGUUUAGUUUUAAAACUA | 1007 | | |
| 1703608 | 3′-C16 | TCUAAACUCUUCAUCUUCAGAAA | 946 | 50 | 70 |
| | | UCUGAAGAUGAAGAGUUUAGA | 984 | | |
| 1726647 | BCY17901 | TCUAAACUCUUCAUCUUCAGAAA | 946 | 5 | 78 |
| | | UCUGAAGAUGAAGAGUUUAGA | 1008 | | |
| 1703668 | 3′-C16 | TUAGUAAAUAUAUUUUCUCUUUC | 947 | 50 | 49 |
| | | AAGAGAAAAUAUAUUUACUAA | 985 | | |
| 1726648 | BCY17901 | TUAGUAAAUAUAUUUUCUCUUUC | 947 | 5 | 55 |
| | | AAGAGAAAAUAUAUUUACUAA | 1009 | | |
| 1669056 | 3′-C16 | TAUAAAUAGAUUCUGUAGCUUUU | 940 | 50 | n/d |
| | | AAGCUACAGAAUCUAUUUAUA | 977 | | |
| 1726638 | BCY17901 | TAUAAAUAGAUUCUGUAGCUUUU | 940 | 5 | 34 |
| | | AAGCUACAGAAUCUAUUUAUA | 1002 | | |

As shown in Table 23, oligomeric duplexes conjugated to a bicycle ligand (BCY17901) were more potent in decreasing human PLN RNA levels in knock-in mouse heart than the same oligomeric duplexes conjugated to a 2-(hydroxymethyl)-6-palmitamidohexyl phosphoryl group, while rank order of potency of duplexes conjugated to BCY17901 was the same as the rank order of potency of duplexes conjugated to a 2-(hydroxymethyl)-6-palmitamidohexyl phosphoryl group.

### Example 6: Potency of 5′-C16, 3′—C16, 5′—BCY17901, or 3′-BCY17901 Conjugated Modified Oligonucleotides and Oligomeric Duplex Compounds Targeting Human PLN

hTfR^{tg/+}/hPLN^{tg+} mice, generated by crossing hTfR^{tg/+} and hPLNs^{tg/+}, were used to determine effects of modified oligonucleotides and oligomeric duplexes on human PLN. hTfR^{tg/+}/hPLNs^{tg/+} mice were divided into groups of 3 mice each. Each mouse received either intravenous (IV) injections of oligomeric duplex compounds or modified oligonucleotides on Days 1, 8, and 15 (a total of 3 treatments) at various doses as indicated. One group of 4 mice received IV injections of PBS as indicated. PBS-injected groups served as the control groups to which oligonucleotide-treated groups were compared. Seven days post final treatment (Day 22), mice were sacrificed and RNA extracted from mouse heart for real-time RTPCR analysis of PLN RNA expression. Human PLN primer probe set ABI53044 (described in Example 5) was used to measure human PLN RNA levels. PLN RNA levels were normalized to total RNA content, as measured by RIBOGREEN®. Results are presented as percent PLN RNA, relative to the amount of PLN in PBS treated animals (% control). Half maximal effective dose (ED₅₀) and the half maximal effective concentration (EC₅₀) of each modified oligonucleotide or oligomeric duplex was calculated using GraphPad Prism 7 software (GraphPad Software, San Diego, CA). ED₅₀ values are presented in mg/kg. EC₅₀ values are presented in ug/g and calculated based on concentration of the antisense oligonucleotide (either modified oligonucleotide or the antisense oligonucleotide of the oligomeric duplex) as measured by mass spectrometry carried out on heart tissue extracted from treated animals. In cases where ED₅₀/EC₅₀ could not be reliably calculated, the values are presented as Not Defined (N.D).

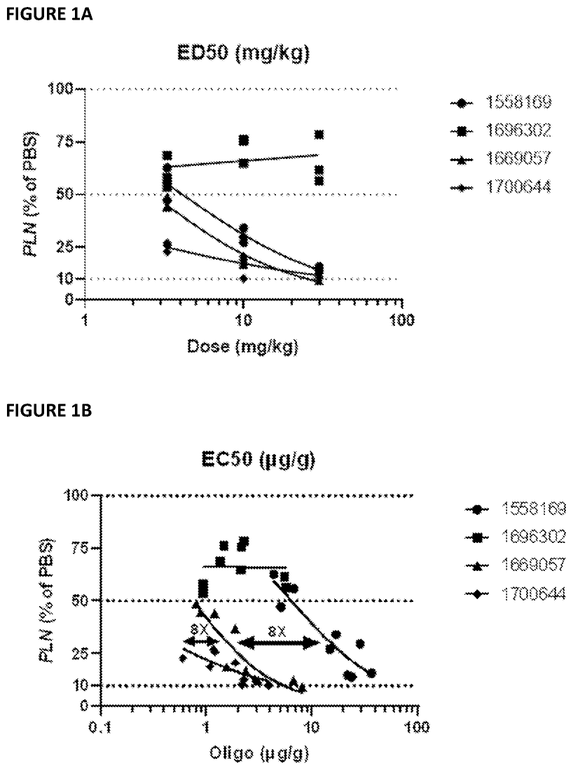

The data is also presented in graphical format in FIGS. 1A and 1B.

TABLE 24
Potency of conjugated modified oligonucleotides and oligomeric duplex compounds

| Compound | | Route of | Dose | PLN RNA (% control) | | |
|---|---|---|---|---|---|---|
| No. | Conjugate | Administration | (mg/kg) | Heart | ED50 (mg/kg) | EC50 (μg/kg) |
| PBS | — | IV | — | 100 | — | — |
| 1558169 | 5′-C16-ASO | IV | 3.3 | 55 | 4.13 | 6.43 |
| | | | 10 | 30 | | |
| | | | 30 | 15 | | |
| 1669057 | 3′-C16-duplex | IV | 3.3 | 46 | 2.69 | 0.75 |
| | | | 10 | 18‡ | | |
| | | | 30 | 11 | | |
| 1696302 | 5′-BCY17901-ASO | IV | 3.3 | 60 | N.D. | N.D. |
| | | | 10 | 72 | | |
| | | | 30 | 66 | | |
| 1700644 | 3′-BCY17901-duplex | IV | 3.3 | 25 | 0.25 | 0.10 |
| | | | 10 | 17 | | |
| | | | 30 | 12 | | |

‡Fewer than 3 samples were available

hTfR^{tg/+}/hPLN^{tg/+} mice, generated by crossing hTfR^{tg/+} and hPLN^{tg/+}, were used to determine effects of oligomeric duplexes on human PLN. hTfR^{tg/+}/hPLN^{tg/+} mice were divided into groups of 3 mice each. Each mouse received either intravenous (IV) or subcutaneous (SC) injections of conjugated duplex compound on Days 1, 8, 15 (a total of 3 treatments) at various doses as indicated. Groups of 2 mice received either IV or SC injections of PBS. PBS-injected groups served as the control groups to which compound-treated groups were compared. Seven days post final treatment (Day 22) mice were sacrificed and RNA extracted from mouse heart for real-time RTPCR analysis of PLN RNA expression. Human PLN primer probe set ABI53044 (described in Example 5) was used to measure human PLN RNA levels. PLN RNA levels were normalized to total RNA content, as measured by RIBOGREEN®. Levels of PLN RNA in hearts of PBS-treated mice administered by IV and SC routes were averaged for comparison to compound treated group. Results are presented as percent PLN RNA, relative to the amount of PLN in PBS treated animals (% control). Half maximal effective dose (ED₅₀) of each oligomeric duplex was calculated using GraphPad Prism 7 software (GraphPad Software, San Diego, CA).

TABLE 25
Potency of BCY17901 conjugated oligomeric duplex compounds

| | | | | PLN RNA | |
|---|---|---|---|---|---|
| | | | | (% control) | |
| Compound | | Route of | Dose | | ED50 |
| No. | Conjugate | Administration | (mg/kg) | Heart | (mg/kg) |
| PBS | — | IV or SC | — | 100 | — |
| 1700644 | 3′-BCY17901 | IV | 0.1 | 84 | 1.04 |
| | | | 0.3 | 70 | |
| | | | 1.0 | 53 | |
| | | | 3.0 | 30 | |
| 1700644 | 3′-BCY17901 | SC | 0.1 | 84 | 0.94 |
| | | | 0.3 | 65 | |
| | | | 1.0 | 60 | |
| | | | 3.0 | 20 | |

hTfR^{tg/+}/hPLN^{tg/tg} mice, generated by crossing hTfR^{tg/+} and hPLN^{tg/tg}, were used to determine effects of a modified oligonucleotide and an oligomeric duplex on human PLN. hTfR^{tg/+}/hPLN^{tg/tg} mice were divided into groups of 3 mice each. Each mouse received subcutaneous (SC) injections of conjugated oligomeric duplex compound or conjugated modified oligonucleotide on Days 1, 7, and 11 (a total of 3 treatments) at various doses as indicated. One group of 4 mice received SC injections of PBS as indicated. PBS-injected groups served as the control groups to which compound-treated groups were compared. Five days post final treatment (Day 16) mice were sacrificed and RNA extracted from mouse heart for real-time RTPCR analysis of PLN RNA expression. Human PLN primer probe set ABI53044 (described in Example 5) was used to measure human PLN RNA levels. PLN RNA levels were normalized to total RNA content, as measured by RIBOGREEN®. Results are presented as percent PLN RNA, relative to the amount of PLN in PBS treated animals (% control). Half maximal effective dose (ED₅₀) of each compound was calculated using GraphPad Prism 7 software (GraphPad Software, San Diego, CA).

TABLE 26
Potency of C16 conjugated modified oligonucleotides
and oligomeric duplex compounds

| | | | | PLN RNA | |
|---|---|---|---|---|---|
| | | | | (% control) | |
| Compound | | Route of | Dose | | ED50 |
| No. | Conjugate | Administration | (mg/kg) | Heart | (mg/kg) |
| PBS | — | SC | — | 100 | — |
| 1558169 | 5′-C16 | SC | 2.7 | 100 | 18.82 |
| | ASO | | 8.0 | 71 | |
| | | | 24.0 | 44 | |
| 1669057 | 3′-C16 | SC | 2.7 | 57 | 3.56 |
| | duplex | | 8.0 | 29 | |
| | | | 24.0 | 11 | |

Results of intravenous and subcutaneous administration of oligonucleotide or oligomeric duplex compounds targeting human PLN RNA shown in Tables 24, 25 and 26 demonstrated both routes of administration yield similar potencies for BCY17901-conjugated oligomeric duplex (Compound No. 1700644) in decreasing hPLN RNA in knock-in mouse heart. Similarly, C16-conjugated oligomeric duplex (Compound No. 1669057) yielded similar potency in decreasing hPLN RNA in knock-in mouse heart when delivered intravenously or subcutaneously. C16-conjugated antisense oligonucleotide (Compound No. 1558169) was more potent in decreasing the amount of hPLN RNA in the mouse heart when administered intravenously as compared to subcutaneous administration.

As shown in Tables 24-26 and FIG. 1, ED50 for a BCY17901-conjugated oligomeric duplex (Compound No. 1700644) was about 2.7-fold lower than the ED50 for a corresponding C16-conjugated oligomeric duplex compound (Compound No. 1669057) and about 4-fold lower than that of a modified antisense oligonucleotide (ASO) conjugated to a C16 group (Compound No. 1558169); estimated EC50 for the antisense strand of the BCY17901-conjugated oligomeric duplex compound (Compound No. 1700644) was about 8-fold lower than the EC50 for the antisense strand of the corresponding C16-conjugated oligomeric duplex compound (Compound No. 1669057) and about 64-fold lower than the EC50 for a modified antisense oligonucleotide (ASO) conjugated to a C16 (Compound No. 1558169). Because of the potency of the BCY17901-conjugated oligomeric duplex (Compound No. 1700644), the four-point dose response shown in Table 25 yielding estimated ED50 1.04 mg/kg was used as a more accurate estimator, and data was also used to estimate EC50 in Table 24. Notably, BCY17901-conjugated antisense oligonucleotide (compound 1696302) failed to demonstrate dose dependent reduction in PLN activity.

### Example 7: Activity of 5′-C16, 3′—C16, 5′—BCY17901, or 3′-BCY17901 Conjugated Oligomeric Duplex Compounds

hPLN^{tg/tg} knock-in mice and hTfR^{tg/+}/hPLN^{tg/tg} knock-in mice were used to determine effects of the conjugated oligomeric duplex compounds on human PLN. hPLN^{tg/tg} and hTfR^{tg/+}/hPLN^{tg/tg} knock-in mice were divided into groups of 3 mice each. Each mouse received a single subcutaneous injection of compound at a dose of either 30 mg/kg or 3 mg/kg. Two groups of 3 mice each received a single subcutaneous injection of PBS, serve as control groups. Seven days post treatment, mice were sacrificed and RNA extracted from mouse heart for real-time RTPCR analysis of PLN RNA expression. Human PLN primer probe set ABI53044 (described in Example 5) was used to measure human PLN RNA levels. PLN RNA levels were normalized to total RNA content, as measured by RIBOGREEN®. Results are presented as percent PLN RNA, relative to the amount of PLN in PBS treated animals (% control).

TABLE 27
Reduction of PLN RNA by conjugated
oligomeric duplex compounds

| | | | | PLN RNA |
|---|---|---|---|---|
| Mouse | Compound | | Dose | (% control) |
| Strain | No. | Conjugate | (mg/kg) | Heart |
| hPLN^{tg/tg} | PBS | — | — | 100 |
| hTfR^{tg/+}/hPLN^{tg/tg} | PBS | — | — | 103 |
| hPLN^{tg/tg} | 1669057 | 3′-C16 | 30 | 47 |
| hPLN^{tg/tg} | 1722804 | 5′-C16 | 30 | 42 |
| hTfR^{tg/+}/hPLN^{tg/tg} | 1700644 | 3′-BCY17901 | 3 | 67 |
| hTfR^{tg/+}/hPLN^{tg/tg} | 1722807 | 5′-BCY17901 | 3 | 50 |
| hPLN^{tg/tg} | 1722807 | 5′-BCY17901 | 3 | 104‡ |

‡fewer than 3 samples were available

As shown in Table 27, compounds having conjugate attached at the 5′ end or 3′ end of the sense strand of the duplex yielded similar activity in decreasing amount of hPLN RNA in hPLN knock-in mice. Oligomeric duplex compounds targeting human PLN RNA are significantly more potent when conjugated to the transferrin receptor 1-binding peptide BCY17901 as compared to a C16 conjugate group, and the activity of the BCY17901-conjugated duplex oligonucleotide compounds in heart in vivo requires human transferrin receptor 1.

### Example 8: Potency of 3′-BCY17901 Conjugated Oligomeric Duplex Compounds Targeting Human PLN

hTfR^{tg/+}/hPLN^{tg/tg} knock-in mice were used to determine effects of the compounds on human PLN. hTfR^{tg/+}/hPLN^{tg/tg} knock-in mice were divided into groups of 3 mice each. Each mouse received a single subcutaneous injection of compound at various doses. One group of 4 mice received a single subcutaneous injection of PBS and served as the control group to which compound-treated groups were compared. Two weeks post treatment, mice were sacrificed and RNA extracted from mouse heart for real-time RTPCR analysis of PLN RNA expression. Human PLN primer probe set ABI53044 (described in Example 5) was used to measure human PLN RNA levels as indicated in Table 28. PLN RNA levels were normalized to total RNA content, as measured by RIBOGREEN®. Results are presented as percent PLN RNA, relative to the amount of PLN in PBS treated animals (% control). Half maximal effective dose (ED₅₀) of each modified oligonucleotide was calculated using GraphPad Prism 7 software (GraphPad Software, San Diego, CA).

TABLE 28
Potency of 3′-BCY17901 conjugated oligomeric duplex compounds

PLN RNA
(% control)

| Compound | Dose | | ED50 |
|---|---|---|---|
| No. | (mg/kg) | Heart | (mg/kg) |
| PBS | — | 100 | — |
| 1700644 | 1 | 81 | 6.15 |
| | 3 | 61 | |
| | 9 | 44 | |
| 1726638 | 1 | 102 | 4.22 |
| | 3 | 59 | |
| | 9 | 25 | |
| 1726640 | 1 | 107 | 8.78 |
| | 3 | 76 | |
| | 9 | 51 | |
| 1726641 | 1 | 100 | 23.83 |
| | 3 | 88 | |
| | 9 | 75 | |
| 1726645 | 1 | 96 | 29.81 |
| | 3 | 90 | |
| | 9 | 75 | |
| 1726648 | 1 | 91 | 11.04 |
| | 3 | 67 | |
| | 9 | 56 | |

As shown in Table 28, 3′ conjugated BCY17901 oligomeric duplex compounds demonstrated dose dependent activity decreasing the amount of hPLN RNA in hPLN knock-in mice.

### Example 9: Activity of 5′-C16 Conjugated Oligomeric Duplex Compounds Targeting Human PLN

hPLN^{tg/tg} knock-in mice were used to determine effects of the oligomeric duplex compounds containing modified oligonucleotides on human PLN. hPLN^{tg/tg} Knock-in mice were divided into groups of 3 mice each. Each mouse received a single subcutaneous injection of compound at a dose of 50 mg/kg. One group of 4 mice received a single subcutaneous injection of PBS and served as the control group to which compound-treated groups were compared. Two weeks post treatment, mice were sacrificed and RNA extracted from mouse heart for real-time RTPCR analysis of PLN RNA expression. Human PLN primer probe set ABI53044 (described in Example 5) was used to measure human PLN RNA levels. PLN RNA levels were normalized to total RNA content, as measured by RIBOGREEN®. Results of evaluation of various chemical modifications of antisense and sense strands of oligonucleotide duplexes targeting human PLN RNA are presented in Table 29 (A-C) as percent human PLN RNA, relative to the amount of human PLN in PBS treated animals (% control). Several compounds were selected for dose-response studies to evaluate potency of the compounds in decreasing human PLN RNA in hPLN knock-in mouse heart cells in the following example.

TABLE 29
Reduction of human PLN RNA by 5′-C16
conjugated oligomeric duplex compounds

| A. | | B. | | C. | |
|---|---|---|---|---|---|
| | PLN RNA | | PLN RNA | | PLN RNA |
| Compound | (% control) | Compound | (% control) | Compound | (% control) |
| No. | Heart | No. | Heart | No. | Heart |
| PBS | 100 | PBS | 100 | PBS | 100 |
| 1722804 | 16 | 1722804 | 14 | 1722804 | 18 |
| 1735533 | 20 | 1739642 | 10 | 1750177 | 12 |
| 1735536 | 82 | 1739646 | 11 | 1750180 | 21 |
| 1735539 | 18 | 1739649 | 15 | 1750207 | 7 |
| 1735542 | 24 | 1739652 | 13 | 1750213 | 18 |
| 1735545 | 18 | 1739655 | 14 | 1750219 | 25 |
| 1735551 | 20 | 1739658 | 22 | 1750225 | 17 |
| 1735554 | 19 | 1739661 | 11‡ | 1750231 | 7 |
| 1735557 | 12 | 1739664 | 4 | 1750237 | 9 |
| 1735560 | 15 | 1739667 | 17 | 1750243 | 9 |
| 1735563 | 17 | 1739670 | 8 | 1750248 | 12 |
| 1735566 | 19 | 1739673 | 19 | 1750254 | 11 |
| 1735569 | 20 | 1739676 | 8 | 1750259 | 13 |
| 1735572 | 54 | 1739679 | 9 | 1750268 | 17 |
| 1735575 | 30 | 1739682 | 12 | 1750274 | 12 |
| 1735578 | 18 | 1739685 | 9 | 1750280 | 17 |
| 1735581 | 17 | 1739688 | 8 | 1750292 | 8 |
| 1735584 | 45 | 1739691 | 10 | 1750301 | 30 |
| 1735587 | 53 | 1739694 | 11 | 1750355 | 21 |
| 1735590 | 59 | 1739697 | 9 | 1750676 | 8 |
| | | 1739700 | 19 | 1750683 | 8 |

‡fewer than 3 samples

### Example 10: Potency of 5′-C16 Conjugated Oligomeric Duplex Compounds Targeting Human PLN

hPLN^{tg/tg} mice were used to determine effects of compounds on human PLN. hPLN^{tg/tg} mice were divided into groups of 3 mice each. Each mouse received a single subcutaneous injection of compound. One group of 4 mice received a single subcutaneous injection of PBS, served as the control group to which compound-treated groups were compared. Two weeks post treatment, mice were sacrificed and RNA extracted from mouse heart for real-time RTPCR analysis of PLN RNA expression. Human PLN primer probe set ABI53044 (described in Example 5) was used to measure human PLN RNA levels. PLN RNA levels were normalized to total RNA content, as measured by RIBOGREEN®. Results are presented in Table 30 as percent PLN RNA, relative to the amount of PLN in PBS treated animals (% control). Half maximal effective dose (ED₅₀) of each modified oligonucleotide was calculated using GraphPad Prism 7 software (GraphPad Software, San Diego, CA). Compounds having reduced fluorine content and/or containing one or more deoxynucleosides were more potent in decreasing the amount of human PLN.

TABLE 30
Reduction of human PLN RNA by 5′-C16 conjugated
oligomeric duplex compounds in hPLN^{tg/tg} mice

| | | |
|---|---|---|
| Compound | Dose | PLN RNA (% control) |

| No. | (mg/kg) | Heart | ED50 (mg/kg) |
|---|---|---|---|
| PBS | — | 100 | — |
| 1722804 | 6 | 87 | 14.41 |
| | 18 | 37 | |
| | 54 | 13 | |
| 1750254 | 6 | 69 | 9.46 |
| | 18 | 23 | |
| | 54 | 8 | |
| 1750274 | 6 | 59 | 9.78 |
| | 18 | 40 | |
| | 54 | 12 | |
| 1750292 | 6 | 37 | 3.76 |
| | 18 | 13 | |
| | 54 | 5 | |
| 1750676 | 6 | 47 | 5.40 |
| | 18 | 16 | |
| | 54 | 7 | |
| 1750683 | 6 | 45 | 4.96 |
| | 18 | 19 | |
| | 54 | 7 | |

### Example 11: Potency of BCY17901-Conjugated Oligomeric Duplex Compounds Targeting Human PLN

Six compounds, directed to three different PLN sites and each containing one or two deoxynucleosides in the antisense strand and low compound fluorine content were analyzed in a 5-point dose response study. hTfR^{tg/+}/hPLN^{tg/tg} knock-in mice were divided into groups of 3 mice each. Each mouse received subcutaneous injections of compound once a week for three weeks (a total of 3 treatments) at various doses indicated in Table 31. One group of 3 mice received subcutaneous injections of PBS once a week for three weeks (a total of 3 treatments) and served as a control group.

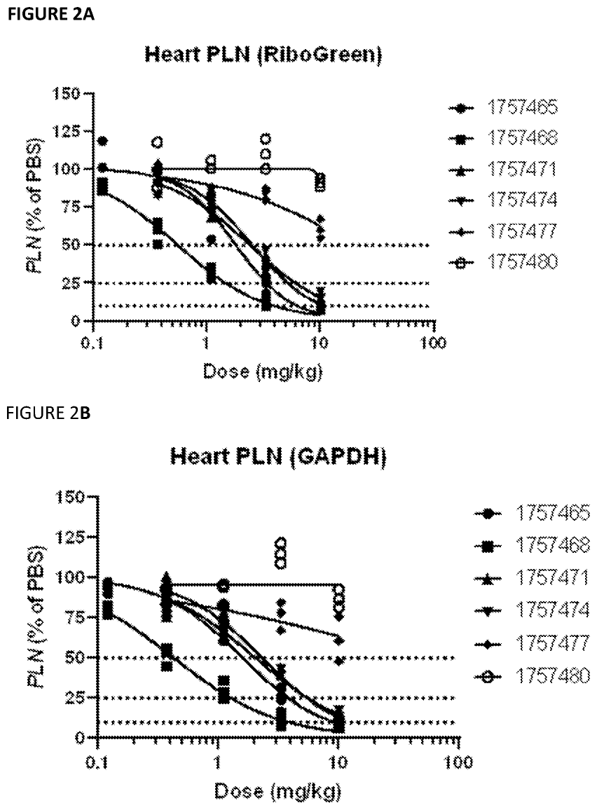

Seven days post final treatment, mice were sacrificed and RNA extracted from mouse heart for real-time RTPCR analysis of PLN RNA expression. Human PLN primer probe set ABI53044 (described in Example 5) was used to measure human PLN RNA levels. PLN RNA levels were normalized to total RNA content, as measured by RIBOGREEN® and were normalized to mouse GAPDH RNA levels. Mouse GAPDH was amplified using mouse primer probe set mGapdh_LTS00102 (forward sequence GGCAAATTCAACGGCACAGT, designated herein as SEQ ID NO: 1059; reverse sequence GGGTCTCGCTCCTGGAAGAT, designated herein as SEQ ID NO: 1060; probe sequence AAGGCCGAGAATGGGAAGCTTGTCATC, designated herein as SEQ ID NO: 1061). Results are presented as percent human PLN RNA, relative to the amount of human PLN RNA in tissue from PBS treated mice (% control). Half maximal effective dose (ED₅₀) of each compound was calculated using GraphPad Prism 7 software (GraphPad Software, San Diego, CA). “N.D.” refers to ED₅₀ values that could not be reliably determined. The data is also presented in graphical format in FIGS. 2A and 2B.

TABLE 31
Reduction of human PLN by 5′-BCY17901-
conjugated oligomeric duplex compounds

| | | PLN RNA (% control) | | | |
|---|---|---|---|---|---|
| Compound | Dose | Ribogreen | | GAPDH | |
| No. | (mg/kg) | Heart | ED50 (mg/kg) | Heart | ED50 (mg/kg) |
| PBS | — | 100 | — | 100 | — |
| 1757465 | 0.12 | 113 | 1.82 | 94 | 1.60 |
| | 0.37 | 96 | | 84 | |
| | 1.1 | 70 | | 66 | |
| | 3.3 | 24 | | 24 | |
| | 10 | 12 | | 11 | |
| 1757468 | 0.12 | 89 | 0.56 | 80 | 0.43 |
| | 0.37 | 59 | | 51 | |
| | 1.1 | 32 | | 30 | |
| | 3.3 | 11 | | 9 | |
| | 10 | 8 | | 6 | |
| 1757471 | 0.37 | 95 | 2.53 | 86 | 2.30 |
| | 1.1 | 79 | | 76 | |
| | 3.3 | 39 | | 38 | |
| | 10 | 12 | | 12 | |
| 1757474 | 0.37 | 88 | 2.40 | 85 | 2.03 |
| | 1.1 | 74 | | 68 | |
| | 3.3 | 39 | | 37 | |
| | 10 | 16 | | 14 | |
| 1757477 | 0.37 | 95 | 20.39 | 86 | 43.23 |
| | 1.1 | 84 | | 77 | |
| | 3.3 | 84 | | 77 | |
| | 10 | 61 | | 62 | |
| 1757480 | 0.37 | 102 | N.D. | 90 | N.D. |
| | 1.1 | 104 | | 91 | |
| | 3.3 | 110 | | 115 | |
| | 10 | 91 | | 87 | |

While single dose activity for each of the compounds compared to their respective parent counterparts appeared to yield similar improvements in initial studies (data not shown), dose response of compounds having the same modifications found certain sites demonstrate particularly significant and unexpected improvements in potency over parent compounds. See FIG. 2. and Table 31.

### Example 12: Activity of Oligomeric Duplexes Targeting Human PLN

hPLN^{tg/tg} knock-in mice were used to determine effects of the oligomeric duplex compounds on human PLN. Mice were divided into groups of 3 mice; and each mouse received a single subcutaneous injection of oligomeric duplex compound at a dose of 45 mg/kg. One group of 4 mice served as a control group and each mouse received a single subcutaneous injection of PBS. Two weeks post treatment, mice were sacrificed and RNA extracted from mouse heart for real-time RTPCR analysis of PLN RNA expression. Human PLN primer probe set AB153044 (described in Example 5) was used to measure human PLN RNA levels. PLN RNA levels were normalized to total RNA content, as measured by RIBOGREEN®. Results are presented in Table 32 as percent PLN RNA, relative to the amount of PLN in PBS treated animals (% control).

TABLE 32
Reduction of human PLN RNA by oligomeric duplex compounds

| | PLN RNA |
|---|---|
| Compound | (% control) |
| No. | Heart |
| PBS | 100 |
| 1722804 | 13‡ |
| 1755748 | 16 |
| 1755751 | 7 |
| 1755757 | 27 |
| 1755763 | 15 |

‡indicates fewer than 3 samples available

### Example 13: Potency of Oligomeric Duplexes Targeting Human PLN

hTfR^{tg/+}/hPLN^{tg/tg} knock-in mice were used to determine effects of the oligomeric duplex compounds on human PLN. hTfR^{tg/+}/hPLN^{tg/tg} knock-in mice were divided into groups of 3 mice each. Each mouse received subcutaneous injections of oligomeric duplex compound on Day 1, Day 5, and Day 8 (a total of 3 treatments) at various doses indicated in Table 33. One group of 4 mice received subcutaneous injections of PBS on Day 1, Day 5, and Day 8 (a total of 3 treatments) and served as the control group to which oligomeric duplex-treated groups were compared. 7 days post final treatment (Day 15), mice were sacrificed and RNA extracted from mouse heart for real-time RTPCR analysis of PLN RNA expression. Human PLN primer probe set ABI53044 (described in Example 5) was used to measure human PLN RNA levels, and PLN RNA levels were normalized to total RNA content, as measured by RIBOGREEN®. Results are presented in Table 33 as percent PLN RNA, relative to the amount of PLN RNA in PBS treated animals (% control). Half maximal effective dose (ED₅₀) of each oligomeric duplex compound was calculated using GraphPad Prism 7 software (GraphPad Software, San Diego, CA).

TABLE 33
Potency of oligomeric duplex compounds

PLN RNA
(% control)

| Compound | Dose | | ED₅₀ |
|---|---|---|---|
| No. | (mg/kg) | Heart | (mg/kg) |
| PBS | — | 100 | — |
| 1757471 | 1.1 | 68 | 1.82 |
| | 3.3 | 28 | |
| | 10 | 12 | |
| 1779744 | 0.12 | 95 | 1.89 |
| | 0.37 | 102 | |
| | 1.1 | 68 | |
| | 3.3 | 28 | |
| | 10 | 11 | |

TABLE 34
Potency of oligomeric duplex compounds

PLN RNA
(% control)

| Compound | Dose | | ED50 |
|---|---|---|---|
| No. | (mg/kg) | Heart | (mg/kg) |
| PBS | — | 100 | — |
| 1757468 | 0.37 | 59 | 0.50 |
| | 1.1 | 27 | |
| | 3.3 | 13 | |
| 1757474 | 0.37 | 102 | 1.57 |
| | 1.1 | 59 | |
| | 3.3 | 26 | |

The potency of oligomeric duplex compounds 1757468 and 1757474 was also evaluated in hTfR^{tg/+}/hPLN^{tg/tg} knock-in mice. Each mouse received subcutaneous injections of oligomeric duplex compound on Day 1, Day 5, and Day 10 (a total of 3 treatments) at various doses indicated in Table 34. One group of 4 mice received subcutaneous injections of PBS on Day 1, Day 5, and Day 10 (a total of 3 treatments) and served as the control group to which oligomeric duplex-treated groups were compared. 6 days post final treatment (Day 16), mice were sacrificed and RNA extracted from mouse heart for real-time RTPCR analysis of PLN RNA expression. Human PLN primer probe set ABI53044 (described in Example 5) was used to measure human PLN RNA levels, and PLN RNA levels were normalized to total RNA content, as measured by RIBOGREEN®. Results are presented in Table 34 as percent PLN RNA, relative to the amount of PLN RNA in PBS treated animals (% control). Half maximal effective dose (ED₅₀) of each oligomeric duplex compound was calculated using GraphPad Prism 7 software (GraphPad Software, San Diego, CA).

### Example 14: Duration of Action Study of Oligomeric Duplex Compounds Targeting Human PLN in hTfR^{tg/+}/hPLN^{tg/tg} Knock-In Mice

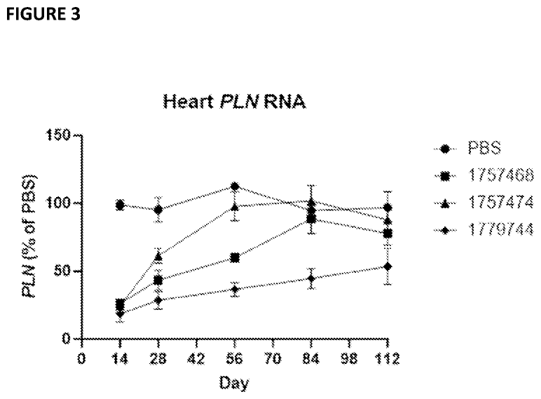

hTfR^{tg/+}/hPLN^{tg/tg} knock-in mice (described herein above) were used to determine effects of the oligomeric duplex compounds on human PLN. Mice, divided into groups of 3 mice each, received subcutaneous injections of oligomeric duplex compound at a dose of either 6 mg/kg or 1.5 mg/kg, as specified in Table 35, on Day 1, 5, and 8 (a total of 3 treatments). One group of 3 mice received subcutaneous injections of PBS on Day 1, 5, and 8 (a total of 3 treatments) served as the control group to which oligomeric duplex-treated groups were compared. Following treatment, mice were sacrificed at various timepoints as indicated in Table 35, and RNA was extracted from mouse heart for real-time RTPCR analysis of PLN RNA expression. Human PLN primer probe set Hs00160179_m1 (Integrated DNA Technologies) was used to measure human PLN RNA levels. PLN RNA levels of each sample were normalized to total RNA content of each sample, as measured by RIBOGREEN®. Results are presented in Table 35 and FIG. 3 as percent PLN RNA, relative to the amount of PLN RNA averaged from all the PBS-treated animals at all timepoints (% control).

TABLE 35
Reduction of human PLN RNA in transgenic mouse
heart by oligomeric duplex compounds

| | | Timepoint | |
|---|---|---|---|
| Compound | Dose | (Days post | PLN RNA |
| No. | (mg/kg) | final dose) | (% control) |
| PBS | — | 14 | 99 |
| | | 28 | 95 |
| | | 56 | 113 |
| | | 84 | 95‡ |
| | | 112 | 97 |
| 1757468 | 1.5 | 14 | 26 |
| | | 28 | 43 |
| | | 56 | 60 |
| | | 84 | 89 |
| | | 112 | 78 |
| 1757474 | 6 | 14 | 24 |
| | | 28 | 61 |
| | | 56 | 98 |
| | | 84 | 102 |
| | | 112 | 88 |
| 1779744 | 6 | 14 | 19 |
| | | 28 | 29 |
| | | 56 | 37 |
| | | 84 | 45 |
| | | 112 | 54 |

‡indicates fewer than 3 samples available

Results presented in Table 35 and FIG. 3 confirm each of the three oligomeric compounds (U.S. Pat. Nos. 1,757,468, 1,757,474, 1,779,744) are potent compounds for reducing human PLN RNA in transgenic mouse heart, with durable effects following compound administration. Compound 1779744 provided the most sustained reduction of PLN RNA levels over time post treatment.

### Example 15: Model of Improved Oligomeric Compounds

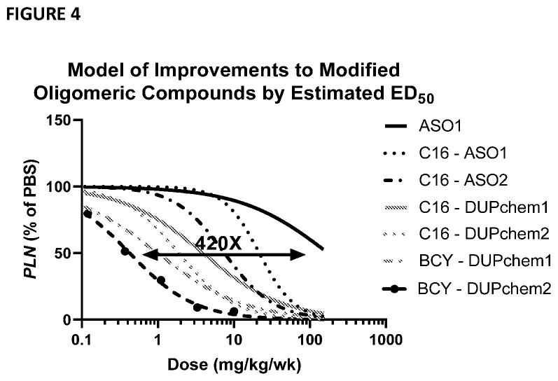

FIG. 4 is a model depicting improvements in activity of compounds targeting human PLN that is based on results of experiments described herein as well as similar experiments not shown. Relative estimated monthly in vivo clinical doses (represented as estimated ED50 values) of different oligomeric compounds targeting PLN for RNA reduction were calculated using dose-response data from knock-in mice studies described herein and similar studies carried out. Compounds represented include a full phosphorothioate 3-10-3 cEt antisense oligonucleotide (ASO1), a C16-conjugated ASO (C16-ASO1), a C16-conjugated ASO having a mixed backbone with reduced phosphorothioate (C16-ASO2), a C16-conjugated first modified oligomeric duplex (C16-DUPchem1), a C16-conjugated second modified oligomeric duplex (C16-DUPchem2), a bicycle ligand-conjugated first modified oligomeric duplex (BCY-DUPchem1), and a bicycle ligand conjugated second modified oligomeric duplex (BCY-DUPchem2). Estimated ED50 for an oligomeric compound comprising a second modified duplex conjugated to a transferrin receptor 1-binding BCY peptide (BCY-DUPchem2) targeted to human PLN is about 420-fold lower than that for an unconjugated single-stranded antisense oligonucleotide (ASO1) targeted to human PLN, about 10-fold lower than an oligomeric compound comprising a modified duplex conjugated to C16 (C16-DUPchem2), and at least two-fold lower than a oligomeric compound having different modifications (BCY-DUPchem1). SEE FIG. 4.

1-123. (canceled)

124. An oligomeric duplex comprising, (1) a first oligomeric compound comprising a first modified oligonucleotide consisting of 18 to 30 contiguous linked nucleosides, wherein the nucleobase sequence of the first modified oligonucleotide comprises at least 21, at least 22, or at least 23 contiguous nucleobases of any one of the nucleobase sequences of SEQ ID NOs: 3-314, 627-782, 939-976, 1033-1038, 1254-1255, or 1258-1287; and (2) a second oligomeric compound comprising a second modified oligonucleotide consisting of 16 to 28 contiguous linked nucleosides that is at least 90% complementary to an equal length portion of the first modified oligonucleotide; wherein: (a) each of the nucleosides of the first modified oligonucleotide and each of the nucleosides of the second modified oligonucleotide comprise a modified sugar moiety or a sugar surrogate, (b) at least one of the modified sugar moiety or sugar surrogate of the first modified oligonucleotide comprises a fluorine, and (c) fewer than 40% of the nucleosides of the first modified oligonucleotide comprise a fluorine; wherein the oligomeric duplex optionally comprises a conjugate group.

125. The oligomeric duplex of claim 124, wherein each nucleoside of the oligomeric duplex comprises (i) a modified sugar moiety or (ii) a sugar moiety independently selected from a 2′-F sugar moiety, a 2′-MOE sugar moiety, a 2′-OMe sugar moiety, a 2′-deoxy sugar moiety, and a 3′-fluoro-hexitol sugar moiety.

126-129. (canceled)

130. The oligomeric duplex of claim 124, wherein the nucleobase sequence of the second modified oligonucleotide comprises least 19, at least 20, or at least 21 contiguous nucleobases of any one of the nucleobase sequences of SEQ ID NOs: 315-626, 783-938, 977-1016, 1027-1032, 1256-1257, or 1288-1290.

131-135. (canceled)

136. The oligomeric duplex of claim 124, wherein (1) the nucleobase of the 3′-terminal nucleoside and/or of the nucleoside immediately 5′ of the 3′-terminal nucleoside of the first modified oligonucleotide and/or of the second modified oligonucleotide is an adenine or thymine; and/or (2) the nucleobase of the 5′-terminal nucleoside of the first modified oligonucleotide is a thymine.

137. The oligomeric duplex of claim 124, wherein the nucleobase of the 3′-terminal nucleoside and the nucleoside immediately 5′ of the 3′-terminal nucleoside of the first modified oligonucleotide is an adenine, and the nucleobase of the 5′-terminal nucleoside of the first modified oligonucleotide is a thymine; and wherein the nucleobase of the 3′-terminal nucleoside of the second modified oligonucleotide is an adenine and the nucleobase of the nucleoside immediately 5′ of the 3′-terminal nucleoside of the second modified oligonucleotide is a thymine or uracil.

138. The oligomeric duplex of claim 124, wherein the first oligomeric compound comprises a two-nucleoside overhang comprising the 3′-terminal nucleoside and the nucleoside immediately 5′ of the 3′-terminal nucleoside of the first modified oligonucleotide

139-140. (canceled)

141. The oligomeric duplex of claim 124, wherein at least one of the modified sugar moieties or sugar surrogates of the second modified oligonucleotide comprises a fluorine and wherein fewer than 40%, fewer than 30%, fewer than 25%, fewer than 20%, fewer than 15%, fewer than 14%, fewer than 13%, fewer than 12%, or fewer than 10%, of the nucleosides of the second modified oligonucleotide comprises a fluorine; and/or
wherein fewer than 30%, fewer than 25%, fewer than 20%, fewer than 15%, fewer than 14%, fewer than 13%, or fewer than 12%, of the total nucleosides in the oligomeric duplex comprise a fluorine.

142-144. (canceled)

145. The oligomeric duplex of claim 124, wherein one or more nucleoside comprising a modified sugar moiety comprising a fluorine or a sugar surrogate comprising a fluorine of the first modified oligonucleotide is/are independently selected from:
i. the second nucleoside counting from the 5′ end,
ii. the fourteenth and sixteenth nucleosides counting from the 5′ end, or
iii. the second, fourteenth and sixteenth nucleosides counting from the 5′ end;
wherein each modified sugar moiety comprising a fluorine or sugar surrogate comprising a fluorine is independently a 2′-fluoro sugar moiety or a 3′-fluoro-hexitol sugar moiety.

146. The oligomeric duplex of claim 124, wherein none of the nucleosides of the second modified oligonucleotide is a 2′-deoxynucleoside and/or none of the nucleosides of the first modified oligonucleotide is a 2′-deoxynucleoside, and/or wherein all of the nucleosides of the second modified oligonucleotide comprise a modified ribosyl sugar moiety.

147. (canceled)

148. The oligomeric duplex of claim 124, wherein one, two, or three of the nucleosides of the first modified oligonucleotide is/are a 2′-deoxynucleoside or wherein no more than 3, nor more than 2, or no more than one nucleoside(s) of the first modified oligonucleotide is/are a 2′-deoxynucleoside(s), or wherein only 3, only 2, or only one nucleoside(s) of the first modified oligonucleotide is/are a 2′-deoxynucleoside(s).

149. (canceled)

150. The oligomeric duplex of claim 124, wherein:
(a) the 5′- and 3′-terminal nucleosides, the nucleoside immediately 3′ of the 5′-terminal nucleoside, and the nucleoside immediately 5′ of the 3′-terminal nucleoside of the second modified oligonucleotide comprise a 2′-MOE sugar moiety, and/or
(b) the 5′- and 3′-terminal nucleosides and the nucleoside immediately 5′ of the 3′-terminal nucleoside of the first modified oligonucleotide comprise a 2′-MOE sugar moiety, and/or
(c) any nucleoside in the first and second modified oligonucleotides that does not comprise a fluorine or a 2′-MOE sugar moiety comprises a 2′-OMe sugar moiety.

151. (canceled)

152. The oligomeric duplex of claim 124, wherein the first oligomeric compound comprises a stabilized phosphate group attached to the 5′-terminal nucleoside of the first modified oligonucleotide.

153-155. (canceled)

156. The oligomeric duplex of claim 124, wherein no more than three of the nucleosides of the first modified oligonucleotide comprise a modified sugar moiety comprising a fluorine or a sugar surrogate comprising a fluorine.

157. The oligomeric duplex of claim 156, wherein the first modified oligonucleotide has a sugar motif (5′ to 3′) selected from among: efyyydyyyyyyyfyfyyyyyee, efyyydyyyyyyyfyfyyyyyyy, e[FHNA]yyydyyyyyyyfyfyyyyyee, e[FHNA]yyydyyyyyyyfyfyyyyyyy, efyydydyyyyyyfyfyyyyyee, efyydydyyyyyyfyfyyyyyyy, e[FHNA]yydydyyyyyyfyfyyyyyee, e[FHNA]yydydyyyyyyfyfyyyyyyy, efyyyyyyyyyyyfyfyyyyyee, efyyyfyyyyyyyfyyyyyyyyy, efyyyfyyyyyyyfydyyyyyee, efyyyfyyyyyyyfydyyyyyyy, efyyyfyyyyyyyfydyyyyyee, efyyyfyyyyyyyfydyyyyyyy, efyydydyyyyyyfyfyyyyye, efyydydyyyyyyfyfyyyyyy, efyyydyyyyyyyfyfyyyyye, efyyydyyyyyyyfyfyyyyyy, efyyyyyyyyyyyfyfyyyyye, efyyyyyyyyyyyfyfyyyyyy, efyydydyyyyyyfyfyyyyy, efyyydyyyyyyyfyfyyyyy, and efyyyyyyyyyyyfyfyyyyy; wherein each “d” represents a 2′-deoxy sugar moiety, each “y” represents a 2′-OMe sugar moiety, each “e” represents a 2′-MOE sugar moiety, each “f” represents a 2′-fluoro sugar moiety, and each “[FHNA]” represents a 3′-fluoro-hexitol sugar moiety.

158-165. (canceled)

166. The oligomeric duplex of claim 124, wherein none of the nucleosides of the first modified oligonucleotide comprises a 2′-deoxy sugar moiety.

167-169. (canceled)

170. The oligomeric duplex of claim 124, wherein no more than three or no more than two of the nucleosides of the second modified oligonucleotide comprise a modified sugar moiety comprising a fluorine or a sugar surrogate comprising a fluorine.

171. The oligomeric duplex of claim 170, wherein the second modified oligonucleotide has a sugar motif (5′ to 3′) selected from among: eeyyyyfyfyfyyyyyyyyee and yyyyyyfyfyfyyyyyyyyyy; eeyyyyyyyffyyyyyyyyee, vvvvvvvvvffvyvvvvvvvv, eeyyvyyffvyyyyyyyee, and yyvvvvvffyyvvvvvvyy; wherein each “y” represents a 2′-OMe sugar moiety, each “e” represents a 2′-MOE sugar moiety, and each “f” represents a 2′-fluoro sugar moiety.

172-174. (canceled)

175. The oligomeric duplex of claim 124, wherein one or more of the internucleoside linkages of the first modified oligonucleotide is/are a modified internucleoside linkage and/or wherein fewer than 50%, fewer than 40%, fewer than 30%, fewer than 20%, or fewer than 10% of the internucleoside linkages of the first modified oligonucleotide are modified internucleoside linkages and/or
wherein one or more of the internucleoside linkages of the second modified oligonucleotide is/are a modified internucleoside linkage(s), and wherein fewer than 50%, fewer than 40%, fewer than 30%, fewer than 20%, or fewer than 10% of the internucleoside linkages of the second modified oligonucleotide are modified internucleoside linkages.

176. The oligomeric duplex of claim 124, wherein the internucleoside linkages between the first and second nucleosides and the second and third nucleosides counting from the 5′ end of the first modified oligonucleotide, and the internucleoside linkages between the first and second nucleosides and the second and third nucleosides counting from the 3′ end of the first modified oligonucleotide are phosphorothioate internucleoside linkages and/or
wherein one or more of the internucleoside linkages of the second modified oligonucleotide is/are a modified internucleoside linkage(s), and wherein fewer than 50%, fewer than 40%, fewer than 30%, fewer than 20%, or fewer than 10% of the internucleoside linkages of the second modified oligonucleotide are modified internucleoside linkages.

177-190. (canceled)

191. The oligomeric duplex of claim 124, wherein the oligomeric duplex comprises a conjugate group comprising a conjugate moiety and a conjugate linker wherein the conjugate moiety comprises a cell-targeting moiety that targets a cell surface receptor.

192-194. (canceled)

195. The oligomeric duplex of claim 191, wherein the oligomeric duplex comprises a conjugate moiety selected from an antibody or fragment thereof, a protein or peptide, and an aptamer, wherein the conjugate moiety that targets type 1 transferrin receptor (TfR1).

196. (canceled)

197. The oligomeric duplex of claim 195, wherein the conjugate moiety is a cyclic protein or cyclic peptide.

198. The oligomeric duplex of claim 195, wherein the conjugate group comprises a bicycle ligand and a conjugate linker, wherein the bicycle ligand comprises a peptide consisting of 13-22 linked amino acids or amino acid mimetics and a molecular scaffold, wherein each of a first, a second, and a third amino acid of the peptide comprises a reactive group, each of which separately forms a bond with the molecular scaffold, thereby forming two peptide loops attached to the molecular scaffold.

199. (canceled)

200. The oligomeric duplex of claim 198 wherein the bicycle ligand comprises an amino acid sequence selected from any one of SEQ ID NO: 1062-1253 and/or wherein the conjugate linker comprises a (bicyclo[6.1.0]nonyne)-formyl (BCN) moiety.

201-203. (canceled)

204. The oligomeric duplex of claim 124, and wherein each cysteine of the peptide forms a covalent bond with the molecular scaffold 1,1′,1″-(1,3,5-triazinane-1,3,5-triyl)tris(2-bromoethanone) (TATB) or the molecular scaffold is 1,1′,1″-(1,3,5-triazinane-1,3,5-triyl)triprop-2-en-1-one (TATA).

205-293. (canceled)

294. A pharmaceutical composition comprising the oligomeric duplex of claim 124, and a pharmaceutically acceptable diluent or carrier.

295-296. (canceled)

297. A method of decreasing the amount of PLN RNA and/or PLN protein in a cell, tissue, organ or subject, comprising contacting the cell, tissue, organ or subject with the oligomeric duplex of claim 124.

298-300. (canceled)

301. A method of preventing or treating a cardiac or cardiovascular disease, disorder, condition or injury associated with heart failure, or postponing a symptom of heart failure, comprising administering to a subject having, or at risk of having, a cardiac or cardiovascular disease, disorder, condition or injury the oligomeric duplex of claim 124 ; wherein the disease, disorder, condition or injury is a cardiomyopathy, cardiac arrythmia, and/or heart failure.

302-329. (canceled)

330. The oligomeric duplex of claim 124, wherein fewer than 20%, fewer than 15%, or fewer than 14% of the nucleosides of the first modified oligonucleotide comprise a fluorine; and/or wherein fewer than 20%, fewer than 15%, fewer than 14%, fewer than 13%, or fewer than 12% of the total nucleosides in the oligomeric duplex comprise a fluorine.


SEQUENCE LISTING (1290 sequences)

SEQ ID NO: 1 - RNA, 3021 nt, Homo sapiens
  source 1..3021: mol_type = mRNA
actttttctcctccacctactgcaactgttcccataaactgggtgacagagtcagaaaactccccagctaaacacccgtaagacttcatacaacacaatactctatactgtgatgatcacagctgccaaggctacctaaaagaagacagttatctcatatttggctgccagctttttatctttctctcgaccacttaaaacttcagacttcctgtcctgctggtatcatggagaaagtccaatacctcactcgctcagctataagaagagcctcaaccattgaaatgcctcaacaagcacgtcaaaagctacagaatctatttatcaatttctgtctcatcttaatatgtctcttgctgatctgtatcatcgtgatgcttctctgaagttctgctacaacctctagatctgcagcttgccacatcagcttaaaatctgtcatcccatgcagacaggaaaacaatattgtataacagaccacttcctgagtagaagagtttctttgtgaaaaggtcaagattaagactaaaacttattgttaccatatgtattcatctgttggatcttgtaaacatgaaaagggctttattttcaaaaattaacttcaaaataagtgtataaaatgcaactgttgatttcctcaacatggctcacaaatttctatcccaaatcttttctgaagatgaagagtttagttttaaaactgcactgccaacaagttcacttcatatataaagcattatttttactcttttgaggtgaatataatttatattacaatgtaaaagcttctttaatactaagtatttttcaggtcttcaccaagtatcaaagtaataacacaaatgaagtgtcattattcaaaatagtccactgactcctcacatctgttatcttattataaagaactatttgtagtaactatcagaatctacattctaaaacagaaattgtattttttctatgccacattaacatcttttaaagttgatgagaatcaagtatggaaaagtaaggccatactcttacataataaaattccttttaagtaattttttcaaagaatcacagaattctagtacatgtaggtaaatcataaatctgttctaagacatatgatcaacagatgagaactggtggttaatatgtgacagtgagattagtcatatcactaatatactaacaacagaatctaatcttcatttaaggcactgtagtgaattatctgagctagagttacctagcttaccatactatatctttggaatcatgaaaccttaagacttcagaatgattttgcaggttgtcttccattccagcctaacatccaatgcaggcaaggaaaataaaagatttccagtgacagaaaaatatattatctcaagtattttttaaaaatatatgaattctctctccaaatattaactaattattagattatattttgaaatgaacttgttggcccatctattacatctacagctgacccttgaacatgggggttaggggagctgacaattcgtgggtccgcaaaatcttaactacctaatagcctactattgaccataaaccttactgataacataaacagtaaattaacacatattttgcgtgttatatgtattatacactatattcctacaataaagtaagctagagaaaatgttatttagaaaatcataagaaagagaaaatatatttactattcattaaatggaagtgggtcaacataaaagtcttcattctcattgtcttcacattgagtaggcagaggaggagaaagatggggaggaagagaaggcgttggtcttgcagtcttgtcttaggggtgtggggagtgggggaaagaatattcatgtataagtggacccttgcaattcaagcccttgttgttcaagggtcaactgtaataggatatagctatttttcttcctctatcaaccaaatggtaagcatctattttgcagtccactctactgagctaaattatagatccagctatgctatttataattattttcttgatgaataaattttcaatttctcctctgaccatttcagaacatcttccaataactcataaaacaactgaagtaaaattgagtgctggaaaatatattcaccaaactttggtaatttaagttgactaaagtttaaaattaagtctaaaatagtttacacctatactgcataatccaacaattttaatttcagttgaagacatgttactaatataactattattaaaagagtagaggatgtgtaattaaccatatcttctaaaacatggttactaaaagaatatgtaacatcaatattgaccttggtttcttacacaagtgttgctaactcaatagtgaaggagacactattaaattttctgaacccatgagagatactagagatggggagtggaaagtgtttggttcagggatatctgaagaacagaagggcagagatttcttaagtgacgcctcatctacaagctggaaattcctaaaaacaagtagaaagcttataaacaacaggtgatacactcacctcactggttttagtaaattaccaatacagaaagtatccctagtcttaaaaacaagtggaaaatttgaactgattagtcatattcctttgattacactgtttgttacaatatttttctcagtaaacagaaataactaatttttttgttcttcattctttgatagaaattaaaatcttattctgtgaggattacagaatactataactcaaattataaagtagaataaactctttaaataattattcttcatcataaagtgtaaagaataagatataagaaaacaatttatttttaaaatttaatatactaaatgctcaaatatgttctactatagaataagttcttatcttaatttacagggcactaaaaacaattttaaaatgcttaatgttgccttttatattttaattggttaagaatatatatttgtttaatgcaaatcagaatcactatattaaaatgaatgttcttgaaaactc

SEQ ID NO: 2 - DNA, 20000 bp, Homo sapiens
  source 1..20000: mol_type = genomic DNA
gataggtaaaatcttcttcctgggtgtctaggagatctaatctaaaaaaagctccttatctttgatgtattgtgaacattacaaaaattgattcaaagcttgcattttactgtatctctgtctaaatattaaaatgctttaatttagtcatcacttatattaacttctggtcaaaaatttccaaaaaccgattctattttgaagcttcttcgccctccggatacaccactacagactggagtttaaatgctcaatgtaaaagattactacatacatcacagtgcccttcacaaaacgttagtaatatagctgggcgcggtggctcacgcctgtaatcccagcactttgggaggccgaggtgggcagatcacgaggtcaggagtttgagaccagcctgggcaatatggtgaaaccccatctctactaaaaatacaaaagttagctgggcgtggtggcacacgcctgtagtctcagctacttgggaggctgaggcaggagaatcgcttgaacccgagagggagaggttgcagtgagctgagatcgcaccattcactcggggctgagtaacagagtgagactccgcacccccttccaaaaaaaattagtaatacttttgaggaacacatttaaaatgctacaaaactatataaaaacaaaagtaaagacatgaactcatgattcagttgaaactgaatactaaatttcatttagagcttagtaaaaataaatttgtaattattttcccatccaagttcaaagacactttacccttaagaatccaccttctaaaggaaaaaagaaaataagtgctaacactaaatgaacactgttgcaggatctctgataatcactatgtacacttcatgtcattaaaccatcacaaatactattccacagattaggctgcttaggaaagttatttgtataaagccatgtgaccaaaaatacattcaaactatagtgtaactccaaatgaagactgtgacaaagcacctcaaaaaaattttctagcctctagctgaggaatcattgaggaacactctactgataacactataactcattaatactcctattaaacaactttctgtttgggcaattaaaaacaatgttcagttaaaactaaaatctaatacttcctagagttccacatgagatcatcaccatcatcgtcatcttcactagctaacccaaaggaacaaggtaaacacaaaaactccccactcttgtaccaatctaaaaagtaaatttttgaatccattttccaactgtgtcacacacagttgttagtatccttctaaggtgtgtatatgcctatgtatactttttaagtaaacttataaacatgtggtaactagggcatattttattctgtctacggcagcttcttggtttatccaactaagtattactaattctattttcttagaatgagaagttctcagaacaatgatctatcacctaaatttcaaatgaatgtgtaactggtagacaaacttaaagcagactttctaaaactattaacagtgctcaagaatgcatctatttacatcttaaaaatttctcagttactatatgctactctaatgaaagctatattcaaattataattttttcccacagcatgccaacagattaggaattataaacagacagatctggatttgaatcctgtctattctttagcagctggttatctctgacaaattactgtaaacctcgttaggcttctcggttttgtattatcaatcatcattggtaactgatagtactgagagatcctcattatgtaaggattgaaagaaaaatggttgtagcactgagaaaagtatcaaacattattctgcaatttccttaacttgggttttacttttaagttgtaaattttcccttggcaatgttcacaggatatctttgctaaaatattatttttcagttagttttaagctgactctttaaatatcaaatgatttgctctagagatggattttctttctttggaagaagccaagatcatttccatttaagggcatgtaagcagctgctgctacacattttagttacccataagaaagtgaagtgggctaagttcactctgtcataagcatttaaaaaattaaaaattacaagtacctcctctaaaattaaagaagagatttaagaattaatattttcgagtgtggtgacttaagtaaatctaacatctaagacaacaaattagataaacagagaagaggagaatgatgcccggctttacaaaaaaattatttccccagttctactgtaagataaactgttctaggaagttcaaaatatcatctcacttactagcatctcatttttatagtaattttcaggaaagcaactgctagattaaatgaggcacatcctttcaaaaacattacaaaacttaaatttcaacacatcaaacctataaagcctcattaactgagatgtaactgatttggaatctgtaattatttcttttttgccataaagttgcccttaatactgcatcagtttattaaaaatttggtaagattgtgtaataatttaatgcaattgtttttaaaaaatcttaaatacttaaagtagcatattgctattttgggggtgttgtatgtatcagtttaaatataagtattactattcacaaatatattgttaaactgctaattgaagaaaacacaaatgaattaaatacatttattagttctttaactgtaagataaaaataattttttaaaacaaaaagcaattagcctgattttctcatactccttctcccatacacacaagaacaagaacaaaataagaaaacaggtataaaattttattgtgtaaaaatatttcactttcactaaaagctaacattatgaagacacgtattaaagcaacagcagcaagaattcaatgaaagttgttttgcttatacctgtgtttatttttctcataattaaaattcctgcagtaactattttagctcttcaacatcttcaccattaatattcaattatctctaagcctgaagatgcatgaatctttaacaaaagctttccaccacctcaagtttacttatttttttcatttatctaccatatactgttttaaattaaaaacaaaagatgaagcacaaagtgttaatgacttttccatacctaaagtaagaagttctaaagtttggttgtgataagactgagactgtggctaaccaatcgaaacttcagaattcctatgtgacatcataagacctccctagaacactttttctcctccacctactgcaactgttcccataaactgggtgacagagtcagaaaactccccagctaaacacccgtaagacttcatacaacacaatactctatactgtgatgatcacagctgccaaggtaagaaacagatttcgctgactttggtaattcttactttctataattcttaattcctataatttctataatctgaacttttaaaatgagttaattttatgaatatcagttcaatcttaggcaataaaatgacatgacaaagtgcttaaatttataaatgcttatccatttagtgacaggaaaaatgagacaactttgaaatgaaaaaggtcatattcatctttgatcctggttactggtttgtcatttaaatttatctttcacttttattttgcttttcaaatgtgaatttctgccaattaaaaaatcttccaattatgttaccatatttggtagtagtggttaatttctccatgcaaaaacaatgacttaattttgttatttattgctctgctgagaaacagtgcagaactgagctatttacatctgaactaatatgaatagtcatttgactacaacatgcacattatcagcatgcctattccaatattatcataatttcatacagtgaataatgaaatggagtttcagaatatataatacaagctacttttaaagtgcccttgaggatgtttcccctctaaagtccagtcaacttttaacaaacatttctaaaaaaattccatgctattaataaattgagttactgatattaaattcaatattgtatcataaagaagtttagaaattttaaaacaatgttcacaaacacaaaaaatctgacgttagagaataatttgatactttaaaaagttattttgacacacgaatattcttaactatcttattttcacacaaatttgattaaatggatttctgcctattaatgcctatagttttgtcaagttattatcctccagtggtactccatttcctctctcggatttagaaagaaaggaaaaaataaaataaatgccaggaatggagatgccacaatcaattttatgacttctgtttgaattctaccctattctttatgttacaatcctcaatttagtaaatgaaacgtttcctcattcttagatctctgtcagggcatacagaaaattacatatattttatttattttgattttcatatttcatatatatgttaaaataaaatttatctctaaaattctcaattcctatgacaaatgttaattctattgctcactacaaataagaactttgtaactctgtaagcagctttccttaagacataaaaaaacataatttttgtctaccactgaggtatcctaattgtgtctccatatctctatactcatatttttcctttgatagtacataaaagtcattcagtgtaggtgtcttatgttatttgtgcatttttattcaaatatctatcaaattacattctcacacttcaaattgggtgttcacgagttattttattgtatcagaattttgctatatcacatattactctaaaaagtacacatacttctggatagagcagaaattatattaaacttacccttagctcactagtatgtctcagttaacagaaattacagttacagagactgctaaaatttcaaatataatgtggtagtaccatacattgtgtcccaactagttcatctataccttaaggtgcaagttaagtattatctttgtttatgaagacatgtggtttcaaagtaagtgctaaaaaaagtgttagatgaaatcaaatggaaaaaatatgaaagatgtttaaattaagaagcattaagttttaaaaacgcatgaaagagccagagtacttgctttactaaaaggcgtgacagcactatcatttcctaaaactcaaatatcaaatccattttatttgtattttagtaggcataaaggaaaatgatcacatttgggtatataatcatatctttaattagtgagattaagaaataataaaatattgcataaatacaatagtttcagaaaattttaatgtattttaatgttttaatgtactcattacacatgtacactaaattatgtaatatattaaatttttaattaaaaaacctaaagtctagttacctctccaaattagacaatatattaccaatcccagaaatgaggagggccaatgaacacttcttttttattcttaaagagcaatttcctaatatactcgtgtacacaaatatttcatttggtcctcttaattatatatttgatactgcatccctattctacaatttaggaaagatgcaaaaaggtgaactgctcaagatcacagaactaagaaaacagggaaaccttggcagaagtttaggttttgtcactaagccacatgcacatcctctatatactgatcagcatggcttaaagtaaaccatgacactgacaaacaaaatttgaaagcccccaaatactgtaaatgatattctaaaaggcaaaaggcaaattcagagaatacaaacttctattttaattaaaagccattccactccatttaaattcaatttatgcatagcattacattcttaatagtctaaattatattctactgatcattttcaaaaccagaaatatacgaaaattacagatgcccttagttattaagcaatacctcctggaaggcaaaattcaaactaactgcacagactttaaaaaaaataacacaccccaatcaatcaaagtttccttttatttgtttcattgctaaaatactcatgccttcaattcatctgatacctaaaatccaagttaacatctcctcaaaactttcacaaaaatcacatttcgatactttattctaggttccctagaatcaagtagtctctttgtacaatttcaaagttcttacccagagtaattctgaagcatgtgttaccaaataaacacatgaaaatgaagcctggatgtacagcatactcatagtcaactgaggacctctagtgcctttgaaaaggtactccatctttagttttcaacccccaacccccgcacaaaaaaaaaggttgactaactcactcacattaatagggggtatatttattaagtggcattcattttaaatccttaagactttctcaatactacctccccatattctcttcccccagaaaagattctgaatttccaactcctacagggttaagaagaatattttacttgtaaatacacttgtaaagcattgtttcaatatttaaaaaacaataatgaaggcaaacctacagacaatatagcaacagcaataagctaaattgtcagttttagagagaaaaatatctctgtgctgttgtttaatgaattctcctcaagacagagctttaacatgagaagcacttcattatgggtcaatttaggaatgagaatatattgatttttatgcatatacaattattttcataaccagacagaagcagcttgaagattctttcaaacagccagtctggaccaacaaagctctggaatgttgctaccacaaagtaacttcaaatagaatgtccaagtttagtattaggcaatctcaaaaggaaaatgactccactggagtgaacttttgtaagccaacagattcatcgtaaatctaatgatgaactgtcctaatttattaccacaagtccaaaaaagggtatatgcacattcacttatggcttatcagtggaaatgtcagaaagtagtagaaaaataaaattccaaacaatccaaatatagacaagtcctgtttattcaatctatagaaaattctaatcttctaaaccaattagatggaaaaaacatcctagagtatatcagactgtagccttagatattacttgaattcaatttaataaagtatttcttcagtactcccatgtttactaaaagaataaacaaaattaaaaggagaaaagcaaaattctaaattctacaaacataaagaatcccaggaatttattatttcagcaaaaggaataccaccaaaaatactgaagtttgagaagttctgcaagaagtgaacatacttaaagtatagatggagattaatgctcagcatatacaacatcattaaagtatcaattgtctagcaagtcataaagcaggccaccaaaagttttcagaagactattgaggttccttccgcacaagaatgcatctgatattacattgctaaaacaggactcactgcagacaaagtaaggactcctgaaggagttgatatatagtagcttccaccaaacatgttgcctaacatggtatatgacattagtcaaaagattagagttgcctaatggcttttctatttccttcttccacctcaaaaaaaaaaccagaataccatcttgaacttgattttttttttaatttccagcgttacttttcttgaggaaaggacgacaaccaaaggatttataaatcccacaaagaatagtaaagaattctttatttttattcttctcattcccctaaaaagtagagtaatcatggatccacagacataagatactctgtggatactttcctgcctctgacatgtaactcaagagacaggagtctggcatttctacacccaaactcctaacaagcacctttcatgttttcaatactcttgattctagggcctagaagacaatttgattttaaaattcatctataaatgaataccactcttgtgtcaaaacaaccaatttatatatattgtcattcttttaataaaagggtggagagcagcttcagtttccatggttgtttaggccatatacaagttcagaaaatcccaaaactatttatgattagactaacagaatgctctaaaaagatcagataacatgaaaataaaactaaactatttttgtatgctttccatcgaaaaagtactctattaaacagcttagctgttaagaaattaaaaaaaaaaaaaaaaaggaaactcatacagtggttgtcagtgcagagaagagaatgataaacatctgacaaccatgccataaattataatgtagtaaaggtcacagacaactccaaaaataggattctgctgttcccacaacaaaggttttcaattctcaatgtatgacaggaagccccatcttctatggataaaatctggcctaattatcatcatacaaatgttggtattcagtagaactaggattccacacatttcagatgataactgtaaaaaattgagcccaataaattaaatgtatatcattaacattttagcgtgtaaagcagatttaagtttaggcataaaatcataaagtagttacaaataactttaggattatggcatatttctaaatctgacaagaaacaggatactatttttggtcatctcctttacacccaatttgtttccagaaacaaactcttctttttcaataacagttagctgttgtaggattataagatcatctttaaagtatgttaactatgctttaaacagtaaagagctcatctctttagggcatatctctgtatttcacaaatactaaaaacccatttatacaaagcctagttcaggttaatactgtgttaatcaggtaaccattaatttaggtgaatcctctaagcctcatttttctcacctgtgatagaaagagacttgaggaaaatattagagcttacatgtccaactaacaaattgttattagaactagttaatctgtcaatcacagcattatttttaaaattgtttaacttattattaaaaatgagggccaggcatggtcattcacacttgtaatcccaggaattacctgagctcaggagtttgagaccagcctgtgcagcacagtgaaaccctgtccctaccaaaaatacaaaaattagccaggcatggtggcgcacacctgtggtctcagctactcaggaggctgaggcagggagatcgctagagcctgggaagttgaggtgcagtgagccacgattgcacaactgcactccagcctgggtgacagagcaagaccctgtcagtcattcattcatccattcataaaaacaattaattaattaaaaatgagatggggtctcattatgttgcccaagctggtctcgaactcctgggctcaaacattcgtcccgcctaggccttccaaagtgttggaattatagatgtgagccaccatgccctgcccacagcattatttttaattaacaattttctaatagttttagtcataaaaatcccttatttaactttagtgatttattgaagcataccaattatgcactaagtactaggatactaaataaacacacagtttctttacttcagaaactatcagtctagctggggatacaaacatgtaaattgaaattgtgctataatctgacagatatcctcatatagataacactaaagccctattaaaaaccagtagaggtgacaaccaattccaccttaataactaatgaaaggtttcactaggaagtgatatttaaactgaaccttaagaatcaactgaagcttgcatggcagacaatcaaaaggtggagaacacattccaggcagagatagaacaacatgtgcaaagaacaaaatcacaaaagggaatggagtatttatggaaaggtgacatgtttcatgtgactgaagtacaacttaccagtgtcaacgtaggggatgactcctgtatatgcttcatgataaactcctggccaactccctcctttcccatgttttgtctcaaaataatttttaaaatttctgaccttttaagaccacttaagcccaaaaacctcacaaaaccatatgttcaaataggaattaccaagtctaattattattcctttcttaactgtattctgctacacatcttcataaaaggtgactactcggccaggtgcagtggctcacgcctgcaatcccagcactttgggaggccgaggcgggtggatcacgaggtcgggagatcgagaccatcctggctaacatggtgaaaccccgtctctactaaaaacacaaaaaaattaggcatggtggcgggcacctgtagtcccagctactcgggaggctaaggcaggagaatggcgtgaacccgggaggtggagcttgcagtgagccgagatcgtgccactgcactccagcctgggcaacacagcaagactgtctcaaaaaaaaaaaaaaaaagtgactactcaaatataaataaaatcttcccataattttccaatccactagatctacaataagcaaatatcaacataagcaaatatcaacataagcaacttaaaggatgaatctcaataaagaacaaaagaaaatctatttatattttacctgaagaattcaaacaaaagtcattaaatatatgtcaagtttaagctgctatgtcccttatttttaacttttgttttaagtttgggggtatgtgtgaaggtttgttacacaggtaaacacatgtcatggggagcttgttgtatgtattacttcatcatcccgttattaagcccagtacccaacagttttctgctcctctgcctcctcccatcctccccgctcaagtagaccccagcgtctgttgtttccttctttgtgttcataagttcttatcatcttgctcccacctgtaagtgagaacctgtggtatttggttttcttttcctatgttagtttgctagggataatgacctccagctccatccatgttcccgcaaaagacatgatatcattcttttttatggctgcatagtattgcatggtgtatatgtaccacattttctttatccagtctgtcactgatgagcacttaggccggttcatgtctttgctattgtgaatagtgctgcaatgaatattcatgggtgtatgtctttacggcagaaagatttacactcctctggatatatacccagtaatgggattgttgggtcgaatggtagttctgcttttataagctgctattttctaagtagaagtgttatagacaacctaaataacagcctttagccacctgtttagacaatgaagaatatatgctagaaaaatacaggtagttaataactctttaaggaaagaaaatttgatagaagttgagtgtttggaactccttgctacagaccaatattatgaagggggacagagtctcaagccaaaaatgcccagtctactatataattcagaatataatagacccaagttgaacactacagaatctaaacaccataaaccatatggtttctttgccaaagtaaatgccgggttctaactagtcatgcctgatacttaccttcccctttgccattctttcagcacttccttctgctcccttcagcactttcctctctcaggaatgggctaacaggttgagatgggcttttatgggatgtataaaaggacttaggagaaaataaaatctgacacctggggtaaggtagtaatatgttgattaacaataatatatcaacaaatgttaacagtcactatgtctggcagttaattactgactttttttctccgtttttcatttttcttatttcaaaattgacacataagtatactgattgaaaacaaaatgtccccaagtgttaataacgattatctctggacagttaatacagttaattttcagtctatatataatttttctgtaatgacatgtaaattcttattttattaatcatcaaagtataggccccacaggcagcaatagcttgtgggatctctgaaagaaggcatgatggaaactactactattaagtagaagtctgtaaataggatatgtctgtggtagtgagataataataattactatgtgttccaagcttgacagtcttcagtacttaacatcaaatcttattaaaatgttacttaagataacattctataaaactataaaatcataaaaaggaataacaatttgaatctatatttttggtaaaacacttcccatttaacactattttattttagattaaaattaagatccagggaacaatttgttgtatgcatttcctttttatgagattggctctggagctattaatacagtgactgatcaaaccaattacatctacagagtaacagatttaaagaaactatatgttagaacttttacccaaaagaataacccaaaacaagctgtaccttaaaccaaatacagcaggtcctcaaataacatcatttctatgtcattttgttataacatttaagtatgtgtggggcttgcatattctccccacatctgtgggactctggtttcctcccatatcccaaaacagtgcacattaggttcactggcatgtttaagttgtcctgggttgagtgtgagtgtgtacatgagtgtatcctgtgatggagtggcctcctatccaaggtgggttcctccctccttgagctgctgggattggctccagccaccagcaccccaaaccagaataagagggttgaaaaatgaatgaatggatgaatacaaattattatcgaataaaaattggtaaagtctacggtgatcatacaaatgcatgacaataaacgctatggcacaaaagcactcagagatcccaccgtatttgaattgtttttgatctgcatggagctggatgtgctcctgacaattttcatttctcaaacatgattccatgtttttaatagaccaccacaaccactgttactcactgattcaccaaatactgggtaattatctttgttggcttttttttttttttcctgggacagagttttgctctgtcacccaggctggagtacagtggcatgatctgggctcaatgcaacccctgcctcctgggctccagcgatcctcccaagtagctaggattacaggcacatgccaccagcctagctaatttttatatttttagtagagacagggtttcaccatgttggccaggctggtctcaaactcctgacctcaggggaactgcccgccttggcctcccaaagtgctgggattacaggcatgagccactgcacctggcctatctggtttttattaatctttcttaaatgtatacatagttcatatttctctcaacgtttaatattagaagtgtttggggtctttattgagaagtttggtgatgtttttgtgaccagaaatatgctataggaacttaactcttgtttacatcaatcagcctagggtaaaaatggttttgttatatgttgttttgcgtaaagtcatggtttccaggaacttatggaagacactgaggattactccaatacttgtagtaaatttgggagaaaaaggcaaggactaaagttactactcctaacacaaatgagacggtcatggtgtgccccatcaaaaaaggagagaaagagagacagacacatagaaacacgtgcacatacacacacacacacacacacacacacacacacacagagagagagagagagagagagagagagggagagagactatagaaacatgatgttataaataacaatagtgctgaggaagatgaattagtgtaaaattgtattttttgttctgaggataggttacatagatgattctaatccatttattatttttacattccaggctacctaaaagaagacagttatctcatatttggctgccagctttttatctttctctcgaccacttaaaacttcagacttcctgtcctgctggtatcatggagaaagtccaatacctcactcgctcagctataagaagagcctcaaccattgaaatgcctcaacaagcacgtcaaaagctacagaatctatttatcaatttctgtctcatcttaatatgtctcttgctgatctgtatcatcgtgatgcttctctgaagttctgctacaacctctagatctgcagcttgccacatcagcttaaaatctgtcatcccatgcagacaggaaaacaatattgtataacagaccacttcctgagtagaagagtttctttgtgaaaaggtcaagattaagactaaaacttattgttaccatatgtattcatctgttggatcttgtaaacatgaaaagggctttattttcaaaaattaacttcaaaataagtgtataaaatgcaactgttgatttcctcaacatggctcacaaatttctatcccaaatcttttctgaagatgaagagtttagttttaaaactgcactgccaacaagttcacttcatatataaagcattatttttactcttttgaggtgaatataatttatattacaatgtaaaagcttctttaatactaagtatttttcaggtcttcaccaagtatcaaagtaataacacaaatgaagtgtcattattcaaaatagtccactgactcctcacatctgttatcttattataaagaactatttgtagtaactatcagaatctacattctaaaacagaaattgtattttttctatgccacattaacatcttttaaagttgatgagaatcaagtatggaaaagtaaggccatactcttacataataaaattccttttaagtaattttttcaaagaatcacagaattctagtacatgtaggtaaatcataaatctgttctaagacatatgatcaacagatgagaactggtggttaatatgtgacagtgagattagtcatatcactaatatactaacaacagaatctaatcttcatttaaggcactgtagtgaattatctgagctagagttacctagcttaccatactatatctttggaatcatgaaaccttaagacttcagaatgattttgcaggttgtcttccattccagcctaacatccaatgcaggcaaggaaaataaaagatttccagtgacagaaaaatatattatctcaagtattttttaaaaatatatgaattctctctccaaatattaactaattattagattatattttgaaatgaacttgttggcccatctattacatctacagctgacccttgaacatgggggttaggggagctgacaattcgtgggtccgcaaaatcttaactacctaatagcctactattgaccataaaccttactgataacataaacagtaaattaacacatattttgcgtgttatatgtattatacactatattcctacaataaagtaagctagagaaaatgttatttagaaaatcataagaaagagaaaatatatttactattcattaaatggaagtgggtcaacataaaagtcttcattctcattgtcttcacattgagtaggcagaggaggagaaagatggggaggaagagaaggcgttggtcttgcagtcttgtcttaggggtgtggggagtgggggaaagaatattcatgtataagtggacccttgcaattcaagcccttgttgttcaagggtcaactgtaataggatatagctatttttcttcctctatcaaccaaatggtaagcatctattttgcagtccactctactgagctaaattatagatccagctatgctatttataattattttcttgatgaataaattttcaatttctcctctgaccatttcagaacatcttccaataactcataaaacaactgaagtaaaattgagtgctggaaaatatattcaccaaactttggtaatttaagttgactaaagtttaaaattaagtctaaaatagtttacacctatactgcataatccaacaattttaatttcagttgaagacatgttactaatataactattattaaaagagtagaggatgtgtaattaaccatatcttctaaaacatggttactaaaagaatatgtaacatcaatattgaccttggtttcttacacaagtgttgctaactcaatagtgaaggagacactattaaattttctgaacccatgagagatactagagatggggagtggaaagtgtttggttcagggatatctgaagaacagaagggcagagatttcttaagtgacgcctcatctacaagctggaaattcctaaaaacaagtagaaagcttataaacaacaggtgatacactcacctcactggttttagtaaattaccaatacagaaagtatccctagtcttaaaaacaagtggaaaatttgaactgattagtcatattcctttgattacactgtttgttacaatatttttctcagtaaacagaaataactaatttttttgttcttcattctttgatagaaattaaaatcttattctgtgaggattacagaatactataactcaaattataaagtagaataaactctttaaataattattcttcatcataaagtgtaaagaataagatataagaaaacaatttatttttaaaatttaatatactaaatgctcaaatatgttctactatagaataagttcttatcttaatttacagggcactaaaaacaattttaaaatgcttaatgttgccttttatattttaattggttaagaatatatatttgtttaatgcaaatcagaatcactatattaaaatgaatgttcttgaaaactcagtggggctgctctataatacacttacaattgatactaacgataaaacgctctaattatagttttaattttataggggtttttttggtgactttccccaaataaaacaaaatcaagacgcccaagtccagacacacaagagggcagacgtatataagataacaaattgctactacatagcgattatttatcttttgattatttttgagacttctggcaaagtaggactataataaattttcagaaaagatctgctcttctgctaagtaacagcacacttcaaaatgtccagaaattttatgctaatattacatagaaaacttgacagatgaaaaaagtgaaacacattactaattttattcattaaaacaggtatataaaagtcctctttaactacctaatgaatgtgtcatatcctaatatagggagtgtcatagtgttcacatctttgaatatataaattatctcaaacagttaaaaaattaaagccagaacagggtacaaaaataagaataaaaataagaaaactttctgttcacaggacgtaacagaatactttaaacactctcaagttataaataaaactttttcattattatttcatgttagacaaaaaacaagtcattttactttaactcattttatcaattttttttttttaaatcaagagacagggtgttactctgtcatgcaggctggaatgcagtcacatgatcattgcacactgcagccttgaactcttggggtgcaaaggattctccaaccttggttcccaagtagctggcactacaggggcacatcaccatatatggctaattctttttattttttgtagagatggggtctcattgcccaggctggtcccaaactcctatcctcaagctatccaccccccatcagcctcccaaagtgttgggattacaggggtgagctaccatgcttggtccaaattttttgtttatacaagggaaaatctccagtaaaatatgtataggtgccaataccataactgttcaaacacccttatgactaaataggcacaaaatttcatctttttaatgactgccacctgaagttcagaaacatcctagggaaaaaaggataaacaacttcatcaataaagaggctactattatttcactactgtctgggagatccatcgtgggcttacaggaatccaaatggatactgccgttacgtgtagaagtgtttatcctccatcccttccaccctaccttccccaagaaaaggaactcaagggggccatggaaagggaaggagtcttcccaaaaagaccagactgtttttcctcctctgtcatagtccctatagagtcaggcagcttaggggttgcagggggtgggtagcataagtgtgatgtactaccgaggaaagagtcaaagactagcagtaggcattacacatggagccttctgtcataaaccatctgagggcataatggcttcttttcattcacaaaggaaaatgattcagagtactacactcctgcactcttgatgatttagcagaaaaccatggaaatcaaggaacaacacacacataagaatgtgtgtctggctcaataattattctggactaagtcctaggaagagcaaaccttcaatgttacaaattaacctggggagtggggagctgtgagaatcaaatgagataaaatactgtgaaagctacagaattctataacatataaatctaaggttttattagattatgactttcagccaaattccttttcttagttgtaaaaatattgttttttcatggtgagtgtttaggagttgtgcttgcaaatcacaatttatcagacggcctggtccttattaaggttcttctctttatgtaaaaattagactaaaagaaaatcagtgtttctatgatttttatcttttttcctttttagagacggggtctggccatgttgcccaggctagtctcaaactcctggactcaggcaattctcctgccttggcctcccaaattgctgggattacaggcgtgcgctacagtgcctggccagaagtcaatgtttcaaagaggcttttaagaaacaactatgatgaaagcttttaaaataaagataatactacttggaggctccaagaaggtatttaacaacttttttctgtagttttctaaggaggaaattagaagggaagcagtcatttaatgattacaacttagtttgtctagttttctttacctgaattcatctttctactccaaaacttaaaagcgctataaagagaccatcccaaataaagttaatcaagaagtagggctttccagggtatatgcaaattttgctctttagttgccttctctctctactatgcactagaaagaagatttaaaaacaaatttatctcaacagtgtgtccccaacctccctaccctcctctgcattgctttgaggctaagaaaaacaccttgtttaaaccagagatttgtccaagcagaataaaaagcatgaatcagtatttactccttgaaatttgcccaattatctctatcacacctactctagtttggaatagcatactcagccccactggacagctccaattctgtgtaaagacaccagctggagaaacaactgcctggatctctgttttcctataatgacaaacatttgaaaactaaaacttgacagtatttgaaaaattaggtttcctatttttcatcacttgaatggataaatcacaaatttctaattcaatcataatgattcttctgttttgattgtgttgtttaaatacataatcaaaattataagaactgtatctaaaataagaataaaggtacatgcgacatgttaagggttctcctatgaatgtagtatgttagggttctatgttttgagatcatactcatatttgtgaggttcctggacaagtgatttccttatcctttcatagcctatgttcatttattctatttgtttttattagtagtagtaatgaataccaattttacatttttactctagggaaaatttgcaattttttatagaattaatttgtttcagaaaattctgatgataaaaaacagatgacaggacttatatcagaaaattagtttccttgttaaaacaagctacagaaaattgtcttggaatagaaacattcccagattaagaaatttacacatttacatgctattccctagaccaactgggggctgtatactatttttctttatatatgatatcatcataaagcactttctataagctgattaatgaatgccttgacacataatttttttt

SEQ ID NO: 3 - RNA, 23 nt, synthetic construct
  source 1..23: mol_type = other RNA
tcatcacagtatagagtattgtg

SEQ ID NO: 4 - RNA, 23 nt, synthetic construct
  source 1..23: mol_type = other RNA
ggtgtttagctggggagttttct

SEQ ID NO: 5 - RNA, 23 nt, synthetic construct
  source 1..23: mol_type = other RNA
tctgactctgtcacccagtttat

SEQ ID NO: 6 - RNA, 23 nt, synthetic construct
  source 1..23: mol_type = other RNA
ggtagccttggcagctgtgatca

SEQ ID NO: 7 - RNA, 23 nt, synthetic construct
  source 1..23: mol_type = other RNA
gtgttgtatgaagtcttacgggt

SEQ ID NO: 8 - RNA, 23 nt, synthetic construct
  source 1..23: mol_type = other RNA
gagataactgtcttcttttaggt

SEQ ID NO: 9 - RNA, 23 nt, synthetic construct
  source 1..23: mol_type = other RNA
tttctccatgataccagcaggac

SEQ ID NO: 10 - RNA, 23 nt, synthetic construct
  source 1..23: mol_type = other RNA
aaaagctggcagccaaatatgag

SEQ ID NO: 11 - RNA, 23 nt, synthetic construct
  source 1..23: mol_type = other RNA
aagtggtcgagagaaagataaaa

SEQ ID NO: 12 - RNA, 23 nt, synthetic construct
  source 1..23: mol_type = other RNA
gacaggaagtctgaagttttaag

SEQ ID NO: 13 - RNA, 23 nt, synthetic construct
  source 1..23: mol_type = other RNA
ttgaggctcttcttatagctgag

SEQ ID NO: 14 - RNA, 23 nt, synthetic construct
  source 1..23: mol_type = other RNA
gagcgagtgaggtattggacttt

SEQ ID NO: 15 - RNA, 23 nt, synthetic construct
  source 1..23: mol_type = other RNA
ttgttgaggcatttcaatggttg

SEQ ID NO: 16 - RNA, 23 nt, synthetic construct
  source 1..23: mol_type = other RNA
ttctgtagcttttgacgtgcttg

SEQ ID NO: 17 - RNA, 23 nt, synthetic construct
  source 1..23: mol_type = other RNA
tagcagaacttcagagaagcatc

SEQ ID NO: 18 - RNA, 23 nt, synthetic construct
  source 1..23: mol_type = other RNA
atcacgatgatacagatcagcaa

SEQ ID NO: 19 - RNA, 23 nt, synthetic construct
  source 1..23: mol_type = other RNA
gacagaaattgataaatagattc

SEQ ID NO: 20 - RNA, 23 nt, synthetic construct
  source 1..23: mol_type = other RNA
caagagacatattaagatgagac

SEQ ID NO: 21 - RNA, 23 nt, synthetic construct
  source 1..23: mol_type = other RNA
agctgcagatctagaggttgtag

SEQ ID NO: 22 - RNA, 23 nt, synthetic construct
  source 1..23: mol_type = other RNA
ctgttatacaatattgttttcct

SEQ ID NO: 23 - RNA, 23 nt, synthetic construct
  source 1..23: mol_type = other RNA
gattttaagctgatgtggcaagc

SEQ ID NO: 24 - RNA, 23 nt, synthetic construct
  source 1..23: mol_type = other RNA
cctgtctgcatgggatgacagat

SEQ ID NO: 25 - RNA, 23 nt, synthetic construct
  source 1..23: mol_type = other RNA
tcttctactcaggaagtggtctg

SEQ ID NO: 26 - RNA, 23 nt, synthetic construct
  source 1..23: mol_type = other RNA
tgaccttttcacaaagaaactct

SEQ ID NO: 27 - RNA, 23 nt, synthetic construct
  source 1..23: mol_type = other RNA
atcaacagttgcattttatacac

SEQ ID NO: 28 - RNA, 23 nt, synthetic construct
  source 1..23: mol_type = other RNA
cacttattttgaagttaattttt

SEQ ID NO: 29 - RNA, 23 nt, synthetic construct
  source 1..23: mol_type = other RNA
ataagttttagtcttaatcttga

SEQ ID NO: 30 - RNA, 23 nt, synthetic construct
  source 1..23: mol_type = other RNA
catgtttacaagatccaacagat

SEQ ID NO: 31 - RNA, 23 nt, synthetic construct
  source 1..23: mol_type = other RNA
gatgaatacatatggtaacaata

SEQ ID NO: 32 - RNA, 23 nt, synthetic construct
  source 1..23: mol_type = other RNA
tttgaaaataaagcccttttcat

SEQ ID NO: 33 - RNA, 23 nt, synthetic construct
  source 1..23: mol_type = other RNA
actaaactcttcatcttcagaaa

SEQ ID NO: 34 - RNA, 23 nt, synthetic construct
  source 1..23: mol_type = other RNA
caaaagagtaaaaataatgcttt

SEQ ID NO: 35 - RNA, 23 nt, synthetic construct
  source 1..23: mol_type = other RNA
ttgtgagccatgttgaggaaatc

SEQ ID NO: 36 - RNA, 23 nt, synthetic construct
  source 1..23: mol_type = other RNA
gttggcagtgcagttttaaaact

SEQ ID NO: 37 - RNA, 23 nt, synthetic construct
  source 1..23: mol_type = other RNA
tttatatatgaagtgaacttgtt

SEQ ID NO: 38 - RNA, 23 nt, synthetic construct
  source 1..23: mol_type = other RNA
aaaagatttgggatagaaatttg

SEQ ID NO: 39 - RNA, 23 nt, synthetic construct
  source 1..23: mol_type = other RNA
tactttgatacttggtgaagacc

SEQ ID NO: 40 - RNA, 23 nt, synthetic construct
  source 1..23: mol_type = other RNA
tgacacttcatttgtgttattac

SEQ ID NO: 41 - RNA, 23 nt, synthetic construct
  source 1..23: mol_type = other RNA
aatataaattatattcacctcaa

SEQ ID NO: 42 - RNA, 23 nt, synthetic construct
  source 1..23: mol_type = other RNA
acctgaaaaatacttagtattaa

SEQ ID NO: 43 - RNA, 23 nt, synthetic construct
  source 1..23: mol_type = other RNA
taaagaagcttttacattgtaat

SEQ ID NO: 44 - RNA, 23 nt, synthetic construct
  source 1..23: mol_type = other RNA
gttattactttgatacttggtga

SEQ ID NO: 45 - RNA, 23 nt, synthetic construct
  source 1..23: mol_type = other RNA
tgtagattctgatagttactaca

SEQ ID NO: 46 - RNA, 23 nt, synthetic construct
  source 1..23: mol_type = other RNA
cagtggactattttgaataatga

SEQ ID NO: 47 - RNA, 23 nt, synthetic construct
  source 1..23: mol_type = other RNA
agataacagatgtgaggagtcag

SEQ ID NO: 48 - RNA, 23 nt, synthetic construct
  source 1..23: mol_type = other RNA
atacaatttctgttttagaatgt

SEQ ID NO: 49 - RNA, 23 nt, synthetic construct
  source 1..23: mol_type = other RNA
acaaatagttctttataataaga

SEQ ID NO: 50 - RNA, 23 nt, synthetic construct
  source 1..23: mol_type = other RNA
gttaatgtggcatagaaaaaata

SEQ ID NO: 51 - RNA, 23 nt, synthetic construct
  source 1..23: mol_type = other RNA
cttacttttccatacttgattct

SEQ ID NO: 52 - RNA, 23 nt, synthetic construct
  source 1..23: mol_type = other RNA
ttattatgtaagagtatggcctt

SEQ ID NO: 53 - RNA, 23 nt, synthetic construct
  source 1..23: mol_type = other RNA
tctcatcaactttaaaagatgtt

SEQ ID NO: 54 - RNA, 23 nt, synthetic construct
  source 1..23: mol_type = other RNA
catacttgattctcatcaacttt

SEQ ID NO: 55 - RNA, 23 nt, synthetic construct
  source 1..23: mol_type = other RNA
aaaattacttaaaaggaatttta

SEQ ID NO: 56 - RNA, 23 nt, synthetic construct
  source 1..23: mol_type = other RNA
gaattctgtgattctttgaaaaa

SEQ ID NO: 57 - RNA, 23 nt, synthetic construct
  source 1..23: mol_type = other RNA
tatgtcttagaacagatttatga

SEQ ID NO: 58 - RNA, 23 nt, synthetic construct
  source 1..23: mol_type = other RNA
attagtgatatgactaatctcac

SEQ ID NO: 59 - RNA, 23 nt, synthetic construct
  source 1..23: mol_type = other RNA
cactgtcacatattaaccaccag

SEQ ID NO: 60 - RNA, 23 nt, synthetic construct
  source 1..23: mol_type = other RNA
ttagattctgttgttagtatatt

SEQ ID NO: 61 - RNA, 23 nt, synthetic construct
  source 1..23: mol_type = other RNA
cagttctcatctgttgatcatat

SEQ ID NO: 62 - RNA, 23 nt, synthetic construct
  source 1..23: mol_type = other RNA
tgatttacctacatgtactagaa

SEQ ID NO: 63 - RNA, 23 nt, synthetic construct
  source 1..23: mol_type = other RNA
tagtatggtaagctaggtaactc

SEQ ID NO: 64 - RNA, 23 nt, synthetic construct
  source 1..23: mol_type = other RNA
tacagtgccttaaatgaagatta

SEQ ID NO: 65 - RNA, 23 nt, synthetic construct
  source 1..23: mol_type = other RNA
ctctagctcagataattcactac

SEQ ID NO: 66 - RNA, 23 nt, synthetic construct
  source 1..23: mol_type = other RNA
gatatagtatggtaagctaggta

SEQ ID NO: 67 - RNA, 23 nt, synthetic construct
  source 1..23: mol_type = other RNA
tgattccaaagatatagtatggt

SEQ ID NO: 68 - RNA, 23 nt, synthetic construct
  source 1..23: mol_type = other RNA
ccaaagatatagtatggtaagct

SEQ ID NO: 69 - RNA, 23 nt, synthetic construct
  source 1..23: mol_type = other RNA
ctgcattggatgttaggctggaa

SEQ ID NO: 70 - RNA, 23 nt, synthetic construct
  source 1..23: mol_type = other RNA
gtttcatgattccaaagatatag

SEQ ID NO: 71 - RNA, 23 nt, synthetic construct
  source 1..23: mol_type = other RNA
aaatcttttattttccttgcctg

SEQ ID NO: 72 - RNA, 23 nt, synthetic construct
  source 1..23: mol_type = other RNA
gaatggaagacaacctgcaaaat

SEQ ID NO: 73 - RNA, 23 nt, synthetic construct
  source 1..23: mol_type = other RNA
atatatttttctgtcactggaaa

SEQ ID NO: 74 - RNA, 23 nt, synthetic construct
  source 1..23: mol_type = other RNA
aatcattctgaagtcttaaggtt

SEQ ID NO: 75 - RNA, 23 nt, synthetic construct
  source 1..23: mol_type = other RNA
tttaaaaaatacttgagataata

SEQ ID NO: 76 - RNA, 23 nt, synthetic construct
  source 1..23: mol_type = other RNA
ctaataattagttaatatttgga

SEQ ID NO: 77 - RNA, 23 nt, synthetic construct
  source 1..23: mol_type = other RNA
ggagagagaattcatatattttt

SEQ ID NO: 78 - RNA, 23 nt, synthetic construct
  source 1..23: mol_type = other RNA
atgtaatagatgggccaacaagt

SEQ ID NO: 79 - RNA, 23 nt, synthetic construct
  source 1..23: mol_type = other RNA
tgttcaagggtcagctgtagatg

SEQ ID NO: 80 - RNA, 23 nt, synthetic construct
  source 1..23: mol_type = other RNA
agttcatttcaaaatataatcta

SEQ ID NO: 81 - RNA, 23 nt, synthetic construct
  source 1..23: mol_type = other RNA
gtcagctcccctaacccccatgt

SEQ ID NO: 82 - RNA, 23 nt, synthetic construct
  source 1..23: mol_type = other RNA
tagttaagattttgcggacccac

SEQ ID NO: 83 - RNA, 23 nt, synthetic construct
  source 1..23: mol_type = other RNA
atttactgtttatgttatcagta

SEQ ID NO: 84 - RNA, 23 nt, synthetic construct
  source 1..23: mol_type = other RNA
gtaaggtttatggtcaatagtag

SEQ ID NO: 85 - RNA, 23 nt, synthetic construct
  source 1..23: mol_type = other RNA
taggctattaggtagttaagatt

SEQ ID NO: 86 - RNA, 23 nt, synthetic construct
  source 1..23: mol_type = other RNA
attttgcggacccacgaattgtc

SEQ ID NO: 87 - RNA, 23 nt, synthetic construct
  source 1..23: mol_type = other RNA
tattttctctttcttatgatttt

SEQ ID NO: 88 - RNA, 23 nt, synthetic construct
  source 1..23: mol_type = other RNA
tagcttactttattgtaggaata

SEQ ID NO: 89 - RNA, 23 nt, synthetic construct
  source 1..23: mol_type = other RNA
catttaatgaatagtaaatatat

SEQ ID NO: 90 - RNA, 23 nt, synthetic construct
  source 1..23: mol_type = other RNA
aacacgcaaaatatgtgttaatt

SEQ ID NO: 91 - RNA, 23 nt, synthetic construct
  source 1..23: mol_type = other RNA
tttctaaataacattttctctag

SEQ ID NO: 92 - RNA, 23 nt, synthetic construct
  source 1..23: mol_type = other RNA
atatagtgtataatacatataac

SEQ ID NO: 93 - RNA, 23 nt, synthetic construct
  source 1..23: mol_type = other RNA
ctcctctgcctactcaatgtgaa

SEQ ID NO: 94 - RNA, 23 nt, synthetic construct
  source 1..23: mol_type = other RNA
ccccacacccctaagacaagact

SEQ ID NO: 95 - RNA, 23 nt, synthetic construct
  source 1..23: mol_type = other RNA
tcttcctccccatctttctcctc

SEQ ID NO: 96 - RNA, 23 nt, synthetic construct
  source 1..23: mol_type = other RNA
cttttatgttgacccacttccat

SEQ ID NO: 97 - RNA, 23 nt, synthetic construct
  source 1..23: mol_type = other RNA
gaagacaatgagaatgaagactt

SEQ ID NO: 98 - RNA, 23 nt, synthetic construct
  source 1..23: mol_type = other RNA
actgcaagaccaacgccttctct

SEQ ID NO: 99 - RNA, 23 nt, synthetic construct
  source 1..23: mol_type = other RNA
tgaatattctttcccccactccc

SEQ ID NO: 100 - RNA, 23 nt, synthetic construct
  source 1..23: mol_type = other RNA
tgcaagggtccacttatacatga

SEQ ID NO: 101 - RNA, 23 nt, synthetic construct
  source 1..23: mol_type = other RNA
atcctattacagttgacccttga

SEQ ID NO: 102 - RNA, 23 nt, synthetic construct
  source 1..23: mol_type = other RNA
agaggaagaaaaatagctatatc

SEQ ID NO: 103 - RNA, 23 nt, synthetic construct
  source 1..23: mol_type = other RNA
atgcttaccatttggttgataga

SEQ ID NO: 104 - RNA, 23 nt, synthetic construct
  source 1..23: mol_type = other RNA
tgaacaacaagggcttgaattgc

SEQ ID NO: 105 - RNA, 23 nt, synthetic construct
  source 1..23: mol_type = other RNA
aaatttattcatcaagaaaataa

SEQ ID NO: 106 - RNA, 23 nt, synthetic construct
  source 1..23: mol_type = other RNA
ttattggaagatgttctgaaatg

SEQ ID NO: 107 - RNA, 23 nt, synthetic construct
  source 1..23: mol_type = other RNA
ggatctataatttagctcagtag

SEQ ID NO: 108 - RNA, 23 nt, synthetic construct
  source 1..23: mol_type = other RNA
taattataaatagcatagctgga

SEQ ID NO: 109 - RNA, 23 nt, synthetic construct
  source 1..23: mol_type = other RNA
atggtcagaggagaaattgaaaa

SEQ ID NO: 110 - RNA, 23 nt, synthetic construct
  source 1..23: mol_type = other RNA
tagagtggactgcaaaatagatg

SEQ ID NO: 111 - RNA, 23 nt, synthetic construct
  source 1..23: mol_type = other RNA
ttaccaaagtttggtgaatatat

SEQ ID NO: 112 - RNA, 23 nt, synthetic construct
  source 1..23: mol_type = other RNA
gattatgcagtataggtgtaaac

SEQ ID NO: 113 - RNA, 23 nt, synthetic construct
  source 1..23: mol_type = other RNA
tattttccagcactcaattttac

SEQ ID NO: 114 - RNA, 23 nt, synthetic construct
  source 1..23: mol_type = other RNA
aactattttagacttaattttaa

SEQ ID NO: 115 - RNA, 23 nt, synthetic construct
  source 1..23: mol_type = other RNA
taaactttagtcaacttaaatta

SEQ ID NO: 116 - RNA, 23 nt, synthetic construct
  source 1..23: mol_type = other RNA
tacttcagttgttttatgagtta

SEQ ID NO: 117 - RNA, 23 nt, synthetic construct
  source 1..23: mol_type = other RNA
aactgaaattaaaattgttggat

SEQ ID NO: 118 - RNA, 23 nt, synthetic construct
  source 1..23: mol_type = other RNA
tatattagtaacatgtcttcaac

SEQ ID NO: 119 - RNA, 23 nt, synthetic construct
  source 1..23: mol_type = other RNA
tagtaaccatgttttagaagata

SEQ ID NO: 120 - RNA, 23 nt, synthetic construct
  source 1..23: mol_type = other RNA
atatggttaattacacatcctct

SEQ ID NO: 121 - RNA, 23 nt, synthetic construct
  source 1..23: mol_type = other RNA
attgatgttacatattcttttag

SEQ ID NO: 122 - RNA, 23 nt, synthetic construct
  source 1..23: mol_type = other RNA
tctactcttttaataatagttat

SEQ ID NO: 123 - RNA, 23 nt, synthetic construct
  source 1..23: mol_type = other RNA
tatccctgaaccaaacactttcc

SEQ ID NO: 124 - RNA, 23 nt, synthetic construct
  source 1..23: mol_type = other RNA
tctctcatgggttcagaaaattt

SEQ ID NO: 125 - RNA, 23 nt, synthetic construct
  source 1..23: mol_type = other RNA
tattgagttagcaacacttgtgt

SEQ ID NO: 126 - RNA, 23 nt, synthetic construct
  source 1..23: mol_type = other RNA
tgtaagaaaccaaggtcaatatt

SEQ ID NO: 127 - RNA, 23 nt, synthetic construct
  source 1..23: mol_type = other RNA
gaatttccagcttgtagatgagg

SEQ ID NO: 128 - RNA, 23 nt, synthetic construct
  source 1..23: mol_type = other RNA
tttaatagtgtctccttcactat

SEQ ID NO: 129 - RNA, 23 nt, synthetic construct
  source 1..23: mol_type = other RNA
tccactccccatctctagtatct

SEQ ID NO: 130 - RNA, 23 nt, synthetic construct
  source 1..23: mol_type = other RNA
actaaaaccagtgaggtgagtgt

SEQ ID NO: 131 - RNA, 23 nt, synthetic construct
  source 1..23: mol_type = other RNA
aggcgtcacttaagaaatctctg

SEQ ID NO: 132 - RNA, 23 nt, synthetic construct
  source 1..23: mol_type = other RNA
tgtatcacctgttgtttataagc

SEQ ID NO: 133 - RNA, 23 nt, synthetic construct
  source 1..23: mol_type = other RNA
ctgcccttctgttcttcagatat

SEQ ID NO: 134 - RNA, 23 nt, synthetic construct
  source 1..23: mol_type = other RNA
agctttctacttgtttttaggaa

SEQ ID NO: 135 - RNA, 23 nt, synthetic construct
  source 1..23: mol_type = other RNA
actttctgtattggtaatttact

SEQ ID NO: 136 - RNA, 23 nt, synthetic construct
  source 1..23: mol_type = other RNA
aatcagttcaaattttccacttg

SEQ ID NO: 137 - RNA, 23 nt, synthetic construct
  source 1..23: mol_type = other RNA
aaaatattgtaacaaacagtgta

SEQ ID NO: 138 - RNA, 23 nt, synthetic construct
  source 1..23: mol_type = other RNA
gttatttctgtttactgagaaaa

SEQ ID NO: 139 - RNA, 23 nt, synthetic construct
  source 1..23: mol_type = other RNA
ttgtttttaagactagggatact

SEQ ID NO: 140 - RNA, 23 nt, synthetic construct
  source 1..23: mol_type = other RNA
gtaatcaaaggaatatgactaat

SEQ ID NO: 141 - RNA, 23 nt, synthetic construct
  source 1..23: mol_type = other RNA
aatgaagaacaaaaaaattagtt

SEQ ID NO: 142 - RNA, 23 nt, synthetic construct
  source 1..23: mol_type = other RNA
tatgatgaagaataattatttaa

SEQ ID NO: 143 - RNA, 23 nt, synthetic construct
  source 1..23: mol_type = other RNA
taaagagtttattctactttata

SEQ ID NO: 144 - RNA, 23 nt, synthetic construct
  source 1..23: mol_type = other RNA
gattttaatttctatcaaagaat

SEQ ID NO: 145 - RNA, 23 nt, synthetic construct
  source 1..23: mol_type = other RNA
ataatttgagttatagtattctg

SEQ ID NO: 146 - RNA, 23 nt, synthetic construct
  source 1..23: mol_type = other RNA
ctgtaatcctcacagaataagat

SEQ ID NO: 147 - RNA, 23 nt, synthetic construct
  source 1..23: mol_type = other RNA
attgtttttagtgccctgtaaat

SEQ ID NO: 148 - RNA, 23 nt, synthetic construct
  source 1..23: mol_type = other RNA
ctatagtagaacatatttgagca

SEQ ID NO: 149 - RNA, 23 nt, synthetic construct
  source 1..23: mol_type = other RNA
aattaagataagaacttattcta

SEQ ID NO: 150 - RNA, 23 nt, synthetic construct
  source 1..23: mol_type = other RNA
atatcttattctttacactttat

SEQ ID NO: 151 - RNA, 23 nt, synthetic construct
  source 1..23: mol_type = other RNA
gcatttagtatattaaattttaa

SEQ ID NO: 152 - RNA, 23 nt, synthetic construct
  source 1..23: mol_type = other RNA
taaaaataaattgttttcttata

SEQ ID NO: 153 - RNA, 23 nt, synthetic construct
  source 1..23: mol_type = other RNA
ttaaacaaatatatattcttaac

SEQ ID NO: 154 - RNA, 23 nt, synthetic construct
  source 1..23: mol_type = other RNA
tgagttttcaagaacattcattt

SEQ ID NO: 155 - RNA, 23 nt, synthetic construct
  source 1..23: mol_type = other RNA
aaccaattaaaatataaaaggca

SEQ ID NO: 156 - RNA, 23 nt, synthetic construct
  source 1..23: mol_type = other RNA
gcaacattaagcattttaaaatt

SEQ ID NO: 157 - RNA, 23 nt, synthetic construct
  source 1..23: mol_type = other RNA
acattcattttaatatagtgatt

SEQ ID NO: 158 - RNA, 23 nt, synthetic construct
  source 1..23: mol_type = other RNA
tatagtgattctgatttgcatta

SEQ ID NO: 159 - RNA, 23 nt, synthetic construct
  source 1..23: mol_type = other RNA
atagagtattgtgttgtatgaag

SEQ ID NO: 160 - RNA, 23 nt, synthetic construct
  source 1..23: mol_type = other RNA
gcagctgtgatcatcacagtata

SEQ ID NO: 161 - RNA, 23 nt, synthetic construct
  source 1..23: mol_type = other RNA
aagtcttacgggtgtttagctgg

SEQ ID NO: 162 - RNA, 23 nt, synthetic construct
  source 1..23: mol_type = other RNA
agccaaatatgagataactgtct

SEQ ID NO: 163 - RNA, 23 nt, synthetic construct
  source 1..23: mol_type = other RNA
tcttcttttaggtagccttggca

SEQ ID NO: 164 - RNA, 23 nt, synthetic construct
  source 1..23: mol_type = other RNA
tggggagttttctgactctgtca

SEQ ID NO: 165 - RNA, 23 nt, synthetic construct
  source 1..23: mol_type = other RNA
atttcaatggttgaggctcttct

SEQ ID NO: 166 - RNA, 23 nt, synthetic construct
  source 1..23: mol_type = other RNA
ctgaagttttaagtggtcgagag

SEQ ID NO: 167 - RNA, 23 nt, synthetic construct
  source 1..23: mol_type = other RNA
ataccagcaggacaggaagtctg

SEQ ID NO: 168 - RNA, 23 nt, synthetic construct
  source 1..23: mol_type = other RNA
gagaaagataaaaagctggcagc

SEQ ID NO: 169 - RNA, 23 nt, synthetic construct
  source 1..23: mol_type = other RNA
ggtattggactttctccatgata

SEQ ID NO: 170 - RNA, 23 nt, synthetic construct
  source 1..23: mol_type = other RNA
tcttatagctgagcgagtgaggt

SEQ ID NO: 171 - RNA, 23 nt, synthetic construct
  source 1..23: mol_type = other RNA
attaagatgagacagaaattgat

SEQ ID NO: 172 - RNA, 23 nt, synthetic construct
  source 1..23: mol_type = other RNA
tacagatcagcaagagacatatt

SEQ ID NO: 173 - RNA, 23 nt, synthetic construct
  source 1..23: mol_type = other RNA
tttgacgtgcttgttgaggcatt

SEQ ID NO: 174 - RNA, 23 nt, synthetic construct
  source 1..23: mol_type = other RNA
ctagaggttgtagcagaacttca

SEQ ID NO: 175 - RNA, 23 nt, synthetic construct
  source 1..23: mol_type = other RNA
tcagagaagcatcacgatgatac

SEQ ID NO: 176 - RNA, 23 nt, synthetic construct
  source 1..23: mol_type = other RNA
gataaatagattctgtagctttt

SEQ ID NO: 177 - RNA, 23 nt, synthetic construct
  source 1..23: mol_type = other RNA
acaaagaaactcttctactcagg

SEQ ID NO: 178 - RNA, 23 nt, synthetic construct
  source 1..23: mol_type = other RNA
tgatgtggcaagctgcagatcta

SEQ ID NO: 179 - RNA, 23 nt, synthetic construct
  source 1..23: mol_type = other RNA
gtcttaatcttgaccttttcaca

SEQ ID NO: 180 - RNA, 23 nt, synthetic construct
  source 1..23: mol_type = other RNA
atattgttttcctgtctgcatgg

SEQ ID NO: 181 - RNA, 23 nt, synthetic construct
  source 1..23: mol_type = other RNA
aggaagtggtctgttatacaata

SEQ ID NO: 182 - RNA, 23 nt, synthetic construct
  source 1..23: mol_type = other RNA
tgggatgacagattttaagctga

SEQ ID NO: 183 - RNA, 23 nt, synthetic construct
  source 1..23: mol_type = other RNA
tatggtaacaataagttttagtc

SEQ ID NO: 184 - RNA, 23 nt, synthetic construct
  source 1..23: mol_type = other RNA
gcattttatacacttattttgaa

SEQ ID NO: 185 - RNA, 23 nt, synthetic construct
  source 1..23: mol_type = other RNA
agatccaacagatgaatacatat

SEQ ID NO: 186 - RNA, 23 nt, synthetic construct
  source 1..23: mol_type = other RNA
aagcccttttcatgtttacaaga

SEQ ID NO: 187 - RNA, 23 nt, synthetic construct
  source 1..23: mol_type = other RNA
gaagttaatttttgaaaataaag

SEQ ID NO: 188 - RNA, 23 nt, synthetic construct
  source 1..23: mol_type = other RNA
tgttgaggaaatcaacagttgca

SEQ ID NO: 189 - RNA, 23 nt, synthetic construct
  source 1..23: mol_type = other RNA
aagtgaacttgttggcagtgcag

SEQ ID NO: 190 - RNA, 23 nt, synthetic construct
  source 1..23: mol_type = other RNA
cagttttaaaactaaactcttca

SEQ ID NO: 191 - RNA, 23 nt, synthetic construct
  source 1..23: mol_type = other RNA
aaaataatgctttatatatgaag

SEQ ID NO: 192 - RNA, 23 nt, synthetic construct
  source 1..23: mol_type = other RNA
atattcacctcaaaagagtaaaa

SEQ ID NO: 193 - RNA, 23 nt, synthetic construct
  source 1..23: mol_type = other RNA
ggatagaaatttgtgagccatgt

SEQ ID NO: 194 - RNA, 23 nt, synthetic construct
  source 1..23: mol_type = other RNA
tcatcttcagaaaagatttggga

SEQ ID NO: 195 - RNA, 23 nt, synthetic construct
  source 1..23: mol_type = other RNA
tacttagtattaaagaagctttt

SEQ ID NO: 196 - RNA, 23 nt, synthetic construct
  source 1..23: mol_type = other RNA
ttttgaataatgacacttcattt

SEQ ID NO: 197 - RNA, 23 nt, synthetic construct
  source 1..23: mol_type = other RNA
tgtgaggagtcagtggactattt

SEQ ID NO: 198 - RNA, 23 nt, synthetic construct
  source 1..23: mol_type = other RNA
cttggtgaagacctgaaaaatac

SEQ ID NO: 199 - RNA, 23 nt, synthetic construct
  source 1..23: mol_type = other RNA
ctttataataagataacagatgt

SEQ ID NO: 200 - RNA, 23 nt, synthetic construct
  source 1..23: mol_type = other RNA
tttacattgtaatataaattata

SEQ ID NO: 201 - RNA, 23 nt, synthetic construct
  source 1..23: mol_type = other RNA
catagaaaaaatacaatttctgt

SEQ ID NO: 202 - RNA, 23 nt, synthetic construct
  source 1..23: mol_type = other RNA
tgttttagaatgtagattctgat

SEQ ID NO: 203 - RNA, 23 nt, synthetic construct
  source 1..23: mol_type = other RNA
tttaaaagatgttaatgtggcat

SEQ ID NO: 204 - RNA, 23 nt, synthetic construct
  source 1..23: mol_type = other RNA
agagtatggccttacttttccat

SEQ ID NO: 205 - RNA, 23 nt, synthetic construct
  source 1..23: mol_type = other RNA
gatagttactacaaatagttctt

SEQ ID NO: 206 - RNA, 23 nt, synthetic construct
  source 1..23: mol_type = other RNA
aaaaggaattttattatgtaaga

SEQ ID NO: 207 - RNA, 23 nt, synthetic construct
  source 1..23: mol_type = other RNA
aacagatttatgatttacctaca

SEQ ID NO: 208 - RNA, 23 nt, synthetic construct
  source 1..23: mol_type = other RNA
tattaaccaccagttctcatctg

SEQ ID NO: 209 - RNA, 23 nt, synthetic construct
  source 1..23: mol_type = other RNA
attctttgaaaaaattacttaaa

SEQ ID NO: 210 - RNA, 23 nt, synthetic construct
  source 1..23: mol_type = other RNA
ctgttgatcatatgtcttagaac

SEQ ID NO: 211 - RNA, 23 nt, synthetic construct
  source 1..23: mol_type = other RNA
tgactaatctcactgtcacatat

SEQ ID NO: 212 - RNA, 23 nt, synthetic construct
  source 1..23: mol_type = other RNA
acatgtactagaattctgtgatt

SEQ ID NO: 213 - RNA, 23 nt, synthetic construct
  source 1..23: mol_type = other RNA
aagtcttaaggtttcatgattcc

SEQ ID NO: 214 - RNA, 23 nt, synthetic construct
  source 1..23: mol_type = other RNA
gataattcactacagtgccttaa

SEQ ID NO: 215 - RNA, 23 nt, synthetic construct
  source 1..23: mol_type = other RNA
taaatgaagattagattctgttg

SEQ ID NO: 216 - RNA, 23 nt, synthetic construct
  source 1..23: mol_type = other RNA
agctaggtaactctagctcagat

SEQ ID NO: 217 - RNA, 23 nt, synthetic construct
  source 1..23: mol_type = other RNA
tgcaaaatcattctgaagtctta

SEQ ID NO: 218 - RNA, 23 nt, synthetic construct
  source 1..23: mol_type = other RNA
ttgttagtatattagtgatatga

SEQ ID NO: 219 - RNA, 23 nt, synthetic construct
  source 1..23: mol_type = other RNA
ttttccttgcctgcattggatgt

SEQ ID NO: 220 - RNA, 23 nt, synthetic construct
  source 1..23: mol_type = other RNA
ttcatatatttttaaaaaatact

SEQ ID NO: 221 - RNA, 23 nt, synthetic construct
  source 1..23: mol_type = other RNA
tgttaggctggaatggaagacaa

SEQ ID NO: 222 - RNA, 23 nt, synthetic construct
  source 1..23: mol_type = other RNA
caacctgcaaaatcattctgaag

SEQ ID NO: 223 - RNA, 23 nt, synthetic construct
  source 1..23: mol_type = other RNA
acttgagataatatatttttctg

SEQ ID NO: 224 - RNA, 23 nt, synthetic construct
  source 1..23: mol_type = other RNA
ctgtcactggaaatcttttattt

SEQ ID NO: 225 - RNA, 23 nt, synthetic construct
  source 1..23: mol_type = other RNA
tgggccaacaagttcatttcaaa

SEQ ID NO: 226 - RNA, 23 nt, synthetic construct
  source 1..23: mol_type = other RNA
gttaatatttggagagagaattc

SEQ ID NO: 227 - RNA, 23 nt, synthetic construct
  source 1..23: mol_type = other RNA
ctaacccccatgttcaagggtca

SEQ ID NO: 228 - RNA, 23 nt, synthetic construct
  source 1..23: mol_type = other RNA
aaaatataatctaataattagtt

SEQ ID NO: 229 - RNA, 23 nt, synthetic construct
  source 1..23: mol_type = other RNA
gaattgtcagctcccctaacccc

SEQ ID NO: 230 - RNA, 23 nt, synthetic construct
  source 1..23: mol_type = other RNA
tcagctgtagatgtaatagatgg

SEQ ID NO: 231 - RNA, 23 nt, synthetic construct
  source 1..23: mol_type = other RNA
taatacatataacacgcaaaata

SEQ ID NO: 232 - RNA, 23 nt, synthetic construct
  source 1..23: mol_type = other RNA
atatgtgttaatttactgtttat

SEQ ID NO: 233 - RNA, 23 nt, synthetic construct
  source 1..23: mol_type = other RNA
tattgtaggaatatagtgtataa

SEQ ID NO: 234 - RNA, 23 nt, synthetic construct
  source 1..23: mol_type = other RNA
tatgttatcagtaaggtttatgg

SEQ ID NO: 235 - RNA, 23 nt, synthetic construct
  source 1..23: mol_type = other RNA
cccacgaattgtcagctccccta

SEQ ID NO: 236 - RNA, 23 nt, synthetic construct
  source 1..23: mol_type = other RNA
tggtcaatagtaggctattaggt

SEQ ID NO: 237 - RNA, 23 nt, synthetic construct
  source 1..23: mol_type = other RNA
agaatgaagacttttatgttgac

SEQ ID NO: 238 - RNA, 23 nt, synthetic construct
  source 1..23: mol_type = other RNA
atagtaaatatattttctctttc

SEQ ID NO: 239 - RNA, 23 nt, synthetic construct
  source 1..23: mol_type = other RNA
ttcttatgattttctaaataaca

SEQ ID NO: 240 - RNA, 23 nt, synthetic construct
  source 1..23: mol_type = other RNA
gacccacttccatttaatgaata

SEQ ID NO: 241 - RNA, 23 nt, synthetic construct
  source 1..23: mol_type = other RNA
tactcaatgtgaagacaatgaga

SEQ ID NO: 242 - RNA, 23 nt, synthetic construct
  source 1..23: mol_type = other RNA
acattttctctagcttactttat

SEQ ID NO: 243 - RNA, 23 nt, synthetic construct
  source 1..23: mol_type = other RNA
gggcttgaattgcaagggtccac

SEQ ID NO: 244 - RNA, 23 nt, synthetic construct
  source 1..23: mol_type = other RNA
catctttctcctcctctgcctac

SEQ ID NO: 245 - RNA, 23 nt, synthetic construct
  source 1..23: mol_type = other RNA
ctaagacaagactgcaagaccaa

SEQ ID NO: 246 - RNA, 23 nt, synthetic construct
  source 1..23: mol_type = other RNA
ttcccccactccccacaccccta

SEQ ID NO: 247 - RNA, 23 nt, synthetic construct
  source 1..23: mol_type = other RNA
cacttatacatgaatattctttc

SEQ ID NO: 248 - RNA, 23 nt, synthetic construct
  source 1..23: mol_type = other RNA
caacgccttctcttcctccccat

SEQ ID NO: 249 - RNA, 23 nt, synthetic construct
  source 1..23: mol_type = other RNA
agttgacccttgaacaacaaggg

SEQ ID NO: 250 - RNA, 23 nt, synthetic construct
  source 1..23: mol_type = other RNA
tttagctcagtagagtggactgc

SEQ ID NO: 251 - RNA, 23 nt, synthetic construct
  source 1..23: mol_type = other RNA
tagcatagctggatctataattt

SEQ ID NO: 252 - RNA, 23 nt, synthetic construct
  source 1..23: mol_type = other RNA
aaatagctatatcctattacagt

SEQ ID NO: 253 - RNA, 23 nt, synthetic construct
  source 1..23: mol_type = other RNA
tttggttgatagaggaagaaaaa

SEQ ID NO: 254 - RNA, 23 nt, synthetic construct
  source 1..23: mol_type = other RNA
tgcaaaatagatgcttaccattt

SEQ ID NO: 255 - RNA, 23 nt, synthetic construct
  source 1..23: mol_type = other RNA
cactcaattttacttcagttgtt

SEQ ID NO: 256 - RNA, 23 nt, synthetic construct
  source 1..23: mol_type = other RNA
atgttctgaaatggtcagaggag

SEQ ID NO: 257 - RNA, 23 nt, synthetic construct
  source 1..23: mol_type = other RNA
gttttatgagttattggaagatg

SEQ ID NO: 258 - RNA, 23 nt, synthetic construct
  source 1..23: mol_type = other RNA
atcaagaaaataattataaatag

SEQ ID NO: 259 - RNA, 23 nt, synthetic construct
  source 1..23: mol_type = other RNA
attgaaaatttattcatcaagaa

SEQ ID NO: 260 - RNA, 23 nt, synthetic construct
  source 1..23: mol_type = other RNA
gagaaattgaaaatttattcatc

SEQ ID NO: 261 - RNA, 23 nt, synthetic construct
  source 1..23: mol_type = other RNA
acatgtcttcaactgaaattaaa

SEQ ID NO: 262 - RNA, 23 nt, synthetic construct
  source 1..23: mol_type = other RNA
aaaattgttggattatgcagtat

SEQ ID NO: 263 - RNA, 23 nt, synthetic construct
  source 1..23: mol_type = other RNA
tcaacttaaattaccaaagtttg

SEQ ID NO: 264 - RNA, 23 nt, synthetic construct
  source 1..23: mol_type = other RNA
gacttaattttaaactttagtca

SEQ ID NO: 265 - RNA, 23 nt, synthetic construct
  source 1..23: mol_type = other RNA
tataggtgtaaactattttagac

SEQ ID NO: 266 - RNA, 23 nt, synthetic construct
  source 1..23: mol_type = other RNA
ttggtgaatatattttccagcac

SEQ ID NO: 267 - RNA, 23 nt, synthetic construct
  source 1..23: mol_type = other RNA
caaggtcaatattgatgttacat

SEQ ID NO: 268 - RNA, 23 nt, synthetic construct
  source 1..23: mol_type = other RNA
catattcttttagtaaccatgtt

SEQ ID NO: 269 - RNA, 23 nt, synthetic construct
  source 1..23: mol_type = other RNA
gttttagaagatatggttaatta

SEQ ID NO: 270 - RNA, 23 nt, synthetic construct
  source 1..23: mol_type = other RNA
atagttatattagtaacatgtct

SEQ ID NO: 271 - RNA, 23 nt, synthetic construct
  source 1..23: mol_type = other RNA
ttacacatcctctactcttttaa

SEQ ID NO: 272 - RNA, 23 nt, synthetic construct
  source 1..23: mol_type = other RNA
taataatagttatattagtaaca

SEQ ID NO: 273 - RNA, 23 nt, synthetic construct
  source 1..23: mol_type = other RNA
atctctagtatctctcatgggtt

SEQ ID NO: 274 - RNA, 23 nt, synthetic construct
  source 1..23: mol_type = other RNA
tagtatctctcatgggttcagaa

SEQ ID NO: 275 - RNA, 23 nt, synthetic construct
  source 1..23: mol_type = other RNA
tctccttcactattgagttagca

SEQ ID NO: 276 - RNA, 23 nt, synthetic construct
  source 1..23: mol_type = other RNA
ccaaacactttccactccccatc

SEQ ID NO: 277 - RNA, 23 nt, synthetic construct
  source 1..23: mol_type = other RNA
gttcagaaaatttaatagtgtct

SEQ ID NO: 278 - RNA, 23 nt, synthetic construct
  source 1..23: mol_type = other RNA
gcaacacttgtgtaagaaaccaa

SEQ ID NO: 279 - RNA, 23 nt, synthetic construct
  source 1..23: mol_type = other RNA
ttgtttttaggaatttccagctt

SEQ ID NO: 280 - RNA, 23 nt, synthetic construct
  source 1..23: mol_type = other RNA
cttgtagatgaggcgtcacttaa

SEQ ID NO: 281 - RNA, 23 nt, synthetic construct
  source 1..23: mol_type = other RNA
taagaaatctctgcccttctgtt

SEQ ID NO: 282 - RNA, 23 nt, synthetic construct
  source 1..23: mol_type = other RNA
tttaggaatttccagcttgtaga

SEQ ID NO: 283 - RNA, 23 nt, synthetic construct
  source 1..23: mol_type = other RNA
gttcttcagatatccctgaacca

SEQ ID NO: 284 - RNA, 23 nt, synthetic construct
  source 1..23: mol_type = other RNA
gttgtttataagctttctacttg

SEQ ID NO: 285 - RNA, 23 nt, synthetic construct
  source 1..23: mol_type = other RNA
gaatatgactaatcagttcaaat

SEQ ID NO: 286 - RNA, 23 nt, synthetic construct
  source 1..23: mol_type = other RNA
gactagggatactttctgtattg

SEQ ID NO: 287 - RNA, 23 nt, synthetic construct
  source 1..23: mol_type = other RNA
acagtgtaatcaaaggaatatga

SEQ ID NO: 288 - RNA, 23 nt, synthetic construct
  source 1..23: mol_type = other RNA
ttggtaatttactaaaaccagtg

SEQ ID NO: 289 - RNA, 23 nt, synthetic construct
  source 1..23: mol_type = other RNA
gtgaggtgagtgtatcacctgtt

SEQ ID NO: 290 - RNA, 23 nt, synthetic construct
  source 1..23: mol_type = other RNA
aattttccacttgtttttaagac

SEQ ID NO: 291 - RNA, 23 nt, synthetic construct
  source 1..23: mol_type = other RNA
tttactgagaaaaatattgtaac

SEQ ID NO: 292 - RNA, 23 nt, synthetic construct
  source 1..23: mol_type = other RNA
aacaaacagtgtaatcaaaggaa

SEQ ID NO: 293 - RNA, 23 nt, synthetic construct
  source 1..23: mol_type = other RNA
tctatcaaagaatgaagaacaaa

SEQ ID NO: 294 - RNA, 23 nt, synthetic construct
  source 1..23: mol_type = other RNA
aataagattttaatttctatcaa

SEQ ID NO: 295 - RNA, 23 nt, synthetic construct
  source 1..23: mol_type = other RNA
aaaaaaattagttatttctgttt

SEQ ID NO: 296 - RNA, 23 nt, synthetic construct
  source 1..23: mol_type = other RNA
cacagaataagattttaatttct

SEQ ID NO: 297 - RNA, 23 nt, synthetic construct
  source 1..23: mol_type = other RNA
ttatttaaagagtttattctact

SEQ ID NO: 298 - RNA, 23 nt, synthetic construct
  source 1..23: mol_type = other RNA
ttatagtattctgtaatcctcac

SEQ ID NO: 299 - RNA, 23 nt, synthetic construct
  source 1..23: mol_type = other RNA
ttgttttcttatatcttattctt

SEQ ID NO: 300 - RNA, 23 nt, synthetic construct
  source 1..23: mol_type = other RNA
aataattatttaaagagtttatt

SEQ ID NO: 301 - RNA, 23 nt, synthetic construct
  source 1..23: mol_type = other RNA
attctactttataatttgagtta

SEQ ID NO: 302 - RNA, 23 nt, synthetic construct
  source 1..23: mol_type = other RNA
ctttacactttatgatgaagaat

SEQ ID NO: 303 - RNA, 23 nt, synthetic construct
  source 1..23: mol_type = other RNA
gcattttaaaattgtttttagtg

SEQ ID NO: 304 - RNA, 23 nt, synthetic construct
  source 1..23: mol_type = other RNA
agaacttattctatagtagaaca

SEQ ID NO: 305 - RNA, 23 nt, synthetic construct
  source 1..23: mol_type = other RNA
ctgtaaattaagataagaactta

SEQ ID NO: 306 - RNA, 23 nt, synthetic construct
  source 1..23: mol_type = other RNA
gtgccctgtaaattaagataaga

SEQ ID NO: 307 - RNA, 23 nt, synthetic construct
  source 1..23: mol_type = other RNA
acatatttgagcatttagtatat

SEQ ID NO: 308 - RNA, 23 nt, synthetic construct
  source 1..23: mol_type = other RNA
tattaaattttaaaaataaattg

SEQ ID NO: 309 - RNA, 23 nt, synthetic construct
  source 1..23: mol_type = other RNA
aatataaaaggcaacattaagca

SEQ ID NO: 310 - RNA, 23 nt, synthetic construct
  source 1..23: mol_type = other RNA
ttcaagaacattcattttaatat

SEQ ID NO: 311 - RNA, 23 nt, synthetic construct
  source 1..23: mol_type = other RNA
atatattcttaaccaattaaaat

SEQ ID NO: 312 - RNA, 23 nt, synthetic construct
  source 1..23: mol_type = other RNA
ctgatttgcattaaacaaatata

SEQ ID NO: 313 - RNA, 23 nt, synthetic construct
  source 1..23: mol_type = other RNA
attttaatatagtgattctgatt

SEQ ID NO: 314 - RNA, 23 nt, synthetic construct
  source 1..23: mol_type = other RNA
aaaaggcaacattaagcatttta

SEQ ID NO: 315 - RNA, 21 nt, synthetic construct
  source 1..21: mol_type = other RNA
caatactctatactgtgatga

SEQ ID NO: 316 - RNA, 21 nt, synthetic construct
  source 1..21: mol_type = other RNA
aaaactccccagctaaacacc

SEQ ID NO: 317 - RNA, 21 nt, synthetic construct
  source 1..21: mol_type = other RNA
aaactgggtgacagagtcaga

SEQ ID NO: 318 - RNA, 21 nt, synthetic construct
  source 1..21: mol_type = other RNA
atcacagctgccaaggctacc

SEQ ID NO: 319 - RNA, 21 nt, synthetic construct
  source 1..21: mol_type = other RNA
ccgtaagacttcatacaacac

SEQ ID NO: 320 - RNA, 21 nt, synthetic construct
  source 1..21: mol_type = other RNA
ctaaaagaagacagttatctc

SEQ ID NO: 321 - RNA, 21 nt, synthetic construct
  source 1..21: mol_type = other RNA
cctgctggtatcatggagaaa

SEQ ID NO: 322 - RNA, 21 nt, synthetic construct
  source 1..21: mol_type = other RNA
catatttggctgccagctttt

SEQ ID NO: 323 - RNA, 21 nt, synthetic construct
  source 1..21: mol_type = other RNA
ttatctttctctcgaccactt

SEQ ID NO: 324 - RNA, 21 nt, synthetic construct
  source 1..21: mol_type = other RNA
taaaacttcagacttcctgtc

SEQ ID NO: 325 - RNA, 21 nt, synthetic construct
  source 1..21: mol_type = other RNA
cagctataagaagagcctcaa

SEQ ID NO: 326 - RNA, 21 nt, synthetic construct
  source 1..21: mol_type = other RNA
agtccaatacctcactcgctc

SEQ ID NO: 327 - RNA, 21 nt, synthetic construct
  source 1..21: mol_type = other RNA
accattgaaatgcctcaacaa

SEQ ID NO: 328 - RNA, 21 nt, synthetic construct
  source 1..21: mol_type = other RNA
agcacgtcaaaagctacagaa

SEQ ID NO: 329 - RNA, 21 nt, synthetic construct
  source 1..21: mol_type = other RNA
tgcttctctgaagttctgcta

SEQ ID NO: 330 - RNA, 21 nt, synthetic construct
  source 1..21: mol_type = other RNA
gctgatctgtatcatcgtgat

SEQ ID NO: 331 - RNA, 21 nt, synthetic construct
  source 1..21: mol_type = other RNA
atctatttatcaatttctgtc

SEQ ID NO: 332 - RNA, 21 nt, synthetic construct
  source 1..21: mol_type = other RNA
ctcatcttaatatgtctcttg

SEQ ID NO: 333 - RNA, 21 nt, synthetic construct
  source 1..21: mol_type = other RNA
acaacctctagatctgcagct

SEQ ID NO: 334 - RNA, 21 nt, synthetic construct
  source 1..21: mol_type = other RNA
gaaaacaatattgtataacag

SEQ ID NO: 335 - RNA, 21 nt, synthetic construct
  source 1..21: mol_type = other RNA
ttgccacatcagcttaaaatc

SEQ ID NO: 336 - RNA, 21 nt, synthetic construct
  source 1..21: mol_type = other RNA
ctgtcatcccatgcagacagg

SEQ ID NO: 337 - RNA, 21 nt, synthetic construct
  source 1..21: mol_type = other RNA
gaccacttcctgagtagaaga

SEQ ID NO: 338 - RNA, 21 nt, synthetic construct
  source 1..21: mol_type = other RNA
agtttctttgtgaaaaggtca

SEQ ID NO: 339 - RNA, 21 nt, synthetic construct
  source 1..21: mol_type = other RNA
gtataaaatgcaactgttgat

SEQ ID NO: 340 - RNA, 21 nt, synthetic construct
  source 1..21: mol_type = other RNA
aaattaacttcaaaataagtg

SEQ ID NO: 341 - RNA, 21 nt, synthetic construct
  source 1..21: mol_type = other RNA
aagattaagactaaaacttat

SEQ ID NO: 342 - RNA, 21 nt, synthetic construct
  source 1..21: mol_type = other RNA
ctgttggatcttgtaaacatg

SEQ ID NO: 343 - RNA, 21 nt, synthetic construct
  source 1..21: mol_type = other RNA
ttgttaccatatgtattcatc

SEQ ID NO: 344 - RNA, 21 nt, synthetic construct
  source 1..21: mol_type = other RNA
gaaaagggctttattttcaaa

SEQ ID NO: 345 - RNA, 21 nt, synthetic construct
  source 1..21: mol_type = other RNA
tctgaagatgaagagtttagt

SEQ ID NO: 346 - RNA, 21 nt, synthetic construct
  source 1..21: mol_type = other RNA
agcattatttttactcttttg

SEQ ID NO: 347 - RNA, 21 nt, synthetic construct
  source 1..21: mol_type = other RNA
tttcctcaacatggctcacaa

SEQ ID NO: 348 - RNA, 21 nt, synthetic construct
  source 1..21: mol_type = other RNA
ttttaaaactgcactgccaac

SEQ ID NO: 349 - RNA, 21 nt, synthetic construct
  source 1..21: mol_type = other RNA
caagttcacttcatatataaa

SEQ ID NO: 350 - RNA, 21 nt, synthetic construct
  source 1..21: mol_type = other RNA
aatttctatcccaaatctttt

SEQ ID NO: 351 - RNA, 21 nt, synthetic construct
  source 1..21: mol_type = other RNA
tcttcaccaagtatcaaagta

SEQ ID NO: 352 - RNA, 21 nt, synthetic construct
  source 1..21: mol_type = other RNA
aataacacaaatgaagtgtca

SEQ ID NO: 353 - RNA, 21 nt, synthetic construct
  source 1..21: mol_type = other RNA
gaggtgaatataatttatatt

SEQ ID NO: 354 - RNA, 21 nt, synthetic construct
  source 1..21: mol_type = other RNA
aatactaagtatttttcaggt

SEQ ID NO: 355 - RNA, 21 nt, synthetic construct
  source 1..21: mol_type = other RNA
tacaatgtaaaagcttcttta

SEQ ID NO: 356 - RNA, 21 nt, synthetic construct
  source 1..21: mol_type = other RNA
accaagtatcaaagtaataac

SEQ ID NO: 357 - RNA, 21 nt, synthetic construct
  source 1..21: mol_type = other RNA
tagtaactatcagaatctaca

SEQ ID NO: 358 - RNA, 21 nt, synthetic construct
  source 1..21: mol_type = other RNA
attattcaaaatagtccactg

SEQ ID NO: 359 - RNA, 21 nt, synthetic construct
  source 1..21: mol_type = other RNA
gactcctcacatctgttatct

SEQ ID NO: 360 - RNA, 21 nt, synthetic construct
  source 1..21: mol_type = other RNA
attctaaaacagaaattgtat

SEQ ID NO: 361 - RNA, 21 nt, synthetic construct
  source 1..21: mol_type = other RNA
ttattataaagaactatttgt

SEQ ID NO: 362 - RNA, 21 nt, synthetic construct
  source 1..21: mol_type = other RNA
ttttttctatgccacattaac

SEQ ID NO: 363 - RNA, 21 nt, synthetic construct
  source 1..21: mol_type = other RNA
aatcaagtatggaaaagtaag

SEQ ID NO: 364 - RNA, 21 nt, synthetic construct
  source 1..21: mol_type = other RNA
ggccatactcttacataataa

SEQ ID NO: 365 - RNA, 21 nt, synthetic construct
  source 1..21: mol_type = other RNA
catcttttaaagttgatgaga

SEQ ID NO: 366 - RNA, 21 nt, synthetic construct
  source 1..21: mol_type = other RNA
agttgatgagaatcaagtatg

SEQ ID NO: 367 - RNA, 21 nt, synthetic construct
  source 1..21: mol_type = other RNA
aaattccttttaagtaatttt

SEQ ID NO: 368 - RNA, 21 nt, synthetic construct
  source 1..21: mol_type = other RNA
tttcaaagaatcacagaattc

SEQ ID NO: 369 - RNA, 21 nt, synthetic construct
  source 1..21: mol_type = other RNA
ataaatctgttctaagacata

SEQ ID NO: 370 - RNA, 21 nt, synthetic construct
  source 1..21: mol_type = other RNA
gagattagtcatatcactaat

SEQ ID NO: 371 - RNA, 21 nt, synthetic construct
  source 1..21: mol_type = other RNA
ggtggttaatatgtgacagtg

SEQ ID NO: 372 - RNA, 21 nt, synthetic construct
  source 1..21: mol_type = other RNA
tatactaacaacagaatctaa

SEQ ID NO: 373 - RNA, 21 nt, synthetic construct
  source 1..21: mol_type = other RNA
atgatcaacagatgagaactg

SEQ ID NO: 374 - RNA, 21 nt, synthetic construct
  source 1..21: mol_type = other RNA
ctagtacatgtaggtaaatca

SEQ ID NO: 375 - RNA, 21 nt, synthetic construct
  source 1..21: mol_type = other RNA
gttacctagcttaccatacta

SEQ ID NO: 376 - RNA, 21 nt, synthetic construct
  source 1..21: mol_type = other RNA
atcttcatttaaggcactgta

SEQ ID NO: 377 - RNA, 21 nt, synthetic construct
  source 1..21: mol_type = other RNA
agtgaattatctgagctagag

SEQ ID NO: 378 - RNA, 21 nt, synthetic construct
  source 1..21: mol_type = other RNA
cctagcttaccatactatatc

SEQ ID NO: 379 - RNA, 21 nt, synthetic construct
  source 1..21: mol_type = other RNA
catactatatctttggaatca

SEQ ID NO: 380 - RNA, 21 nt, synthetic construct
  source 1..21: mol_type = other RNA
cttaccatactatatctttgg

SEQ ID NO: 381 - RNA, 21 nt, synthetic construct
  source 1..21: mol_type = other RNA
ccagcctaacatccaatgcag

SEQ ID NO: 382 - RNA, 21 nt, synthetic construct
  source 1..21: mol_type = other RNA
atatctttggaatcatgaaac

SEQ ID NO: 383 - RNA, 21 nt, synthetic construct
  source 1..21: mol_type = other RNA
ggcaaggaaaataaaagattt

SEQ ID NO: 384 - RNA, 21 nt, synthetic construct
  source 1..21: mol_type = other RNA
tttgcaggttgtcttccattc

SEQ ID NO: 385 - RNA, 21 nt, synthetic construct
  source 1..21: mol_type = other RNA
tccagtgacagaaaaatatat

SEQ ID NO: 386 - RNA, 21 nt, synthetic construct
  source 1..21: mol_type = other RNA
ccttaagacttcagaatgatt

SEQ ID NO: 387 - RNA, 21 nt, synthetic construct
  source 1..21: mol_type = other RNA
ttatctcaagtattttttaaa

SEQ ID NO: 388 - RNA, 21 nt, synthetic construct
  source 1..21: mol_type = other RNA
caaatattaactaattattag

SEQ ID NO: 389 - RNA, 21 nt, synthetic construct
  source 1..21: mol_type = other RNA
aaatatatgaattctctctcc

SEQ ID NO: 390 - RNA, 21 nt, synthetic construct
  source 1..21: mol_type = other RNA
ttgttggcccatctattacat

SEQ ID NO: 391 - RNA, 21 nt, synthetic construct
  source 1..21: mol_type = other RNA
tctacagctgacccttgaaca

SEQ ID NO: 392 - RNA, 21 nt, synthetic construct
  source 1..21: mol_type = other RNA
gattatattttgaaatgaact

SEQ ID NO: 393 - RNA, 21 nt, synthetic construct
  source 1..21: mol_type = other RNA
atgggggttaggggagctgac

SEQ ID NO: 394 - RNA, 21 nt, synthetic construct
  source 1..21: mol_type = other RNA
gggtccgcaaaatcttaacta

SEQ ID NO: 395 - RNA, 21 nt, synthetic construct
  source 1..21: mol_type = other RNA
ctgataacataaacagtaaat

SEQ ID NO: 396 - RNA, 21 nt, synthetic construct
  source 1..21: mol_type = other RNA
actattgaccataaaccttac

SEQ ID NO: 397 - RNA, 21 nt, synthetic construct
  source 1..21: mol_type = other RNA
tcttaactacctaatagccta

SEQ ID NO: 398 - RNA, 21 nt, synthetic construct
  source 1..21: mol_type = other RNA
caattcgtgggtccgcaaaat

SEQ ID NO: 399 - RNA, 21 nt, synthetic construct
  source 1..21: mol_type = other RNA
aatcataagaaagagaaaata

SEQ ID NO: 400 - RNA, 21 nt, synthetic construct
  source 1..21: mol_type = other RNA
ttcctacaataaagtaagcta

SEQ ID NO: 401 - RNA, 21 nt, synthetic construct
  source 1..21: mol_type = other RNA
atatttactattcattaaatg

SEQ ID NO: 402 - RNA, 21 nt, synthetic construct
  source 1..21: mol_type = other RNA
ttaacacatattttgcgtgtt

SEQ ID NO: 403 - RNA, 21 nt, synthetic construct
  source 1..21: mol_type = other RNA
agagaaaatgttatttagaaa

SEQ ID NO: 404 - RNA, 21 nt, synthetic construct
  source 1..21: mol_type = other RNA
tatatgtattatacactatat

SEQ ID NO: 405 - RNA, 21 nt, synthetic construct
  source 1..21: mol_type = other RNA
cacattgagtaggcagaggag

SEQ ID NO: 406 - RNA, 21 nt, synthetic construct
  source 1..21: mol_type = other RNA
tcttgtcttaggggtgtgggg

SEQ ID NO: 407 - RNA, 21 nt, synthetic construct
  source 1..21: mol_type = other RNA
ggagaaagatggggaggaaga

SEQ ID NO: 408 - RNA, 21 nt, synthetic construct
  source 1..21: mol_type = other RNA
ggaagtgggtcaacataaaag

SEQ ID NO: 409 - RNA, 21 nt, synthetic construct
  source 1..21: mol_type = other RNA
gtcttcattctcattgtcttc

SEQ ID NO: 410 - RNA, 21 nt, synthetic construct
  source 1..21: mol_type = other RNA
agaaggcgttggtcttgcagt

SEQ ID NO: 411 - RNA, 21 nt, synthetic construct
  source 1..21: mol_type = other RNA
gagtgggggaaagaatattca

SEQ ID NO: 412 - RNA, 21 nt, synthetic construct
  source 1..21: mol_type = other RNA
atgtataagtggacccttgca

SEQ ID NO: 413 - RNA, 21 nt, synthetic construct
  source 1..21: mol_type = other RNA
aagggtcaactgtaataggat

SEQ ID NO: 414 - RNA, 21 nt, synthetic construct
  source 1..21: mol_type = other RNA
tatagctatttttcttcctct

SEQ ID NO: 415 - RNA, 21 nt, synthetic construct
  source 1..21: mol_type = other RNA
tatcaaccaaatggtaagcat

SEQ ID NO: 416 - RNA, 21 nt, synthetic construct
  source 1..21: mol_type = other RNA
aattcaagcccttgttgttca

SEQ ID NO: 417 - RNA, 21 nt, synthetic construct
  source 1..21: mol_type = other RNA
attttcttgatgaataaattt

SEQ ID NO: 418 - RNA, 21 nt, synthetic construct
  source 1..21: mol_type = other RNA
tttcagaacatcttccaataa

SEQ ID NO: 419 - RNA, 21 nt, synthetic construct
  source 1..21: mol_type = other RNA
actgagctaaattatagatcc

SEQ ID NO: 420 - RNA, 21 nt, synthetic construct
  source 1..21: mol_type = other RNA
cagctatgctatttataatta

SEQ ID NO: 421 - RNA, 21 nt, synthetic construct
  source 1..21: mol_type = other RNA
ttcaatttctcctctgaccat

SEQ ID NO: 422 - RNA, 21 nt, synthetic construct
  source 1..21: mol_type = other RNA
tctattttgcagtccactcta

SEQ ID NO: 423 - RNA, 21 nt, synthetic construct
  source 1..21: mol_type = other RNA
atattcaccaaactttggtaa

SEQ ID NO: 424 - RNA, 21 nt, synthetic construct
  source 1..21: mol_type = other RNA
ttacacctatactgcataatc

SEQ ID NO: 425 - RNA, 21 nt, synthetic construct
  source 1..21: mol_type = other RNA
aaaattgagtgctggaaaata

SEQ ID NO: 426 - RNA, 21 nt, synthetic construct
  source 1..21: mol_type = other RNA
aaaattaagtctaaaatagtt

SEQ ID NO: 427 - RNA, 21 nt, synthetic construct
  source 1..21: mol_type = other RNA
atttaagttgactaaagttta

SEQ ID NO: 428 - RNA, 21 nt, synthetic construct
  source 1..21: mol_type = other RNA
actcataaaacaactgaagta

SEQ ID NO: 429 - RNA, 21 nt, synthetic construct
  source 1..21: mol_type = other RNA
ccaacaattttaatttcagtt

SEQ ID NO: 430 - RNA, 21 nt, synthetic construct
  source 1..21: mol_type = other RNA
tgaagacatgttactaatata

SEQ ID NO: 431 - RNA, 21 nt, synthetic construct
  source 1..21: mol_type = other RNA
tcttctaaaacatggttacta

SEQ ID NO: 432 - RNA, 21 nt, synthetic construct
  source 1..21: mol_type = other RNA
aggatgtgtaattaaccatat

SEQ ID NO: 433 - RNA, 21 nt, synthetic construct
  source 1..21: mol_type = other RNA
aaaagaatatgtaacatcaat

SEQ ID NO: 434 - RNA, 21 nt, synthetic construct
  source 1..21: mol_type = other RNA
aactattattaaaagagtaga

SEQ ID NO: 435 - RNA, 21 nt, synthetic construct
  source 1..21: mol_type = other RNA
aaagtgtttggttcagggata

SEQ ID NO: 436 - RNA, 21 nt, synthetic construct
  source 1..21: mol_type = other RNA
attttctgaacccatgagaga

SEQ ID NO: 437 - RNA, 21 nt, synthetic construct
  source 1..21: mol_type = other RNA
acaagtgttgctaactcaata

SEQ ID NO: 438 - RNA, 21 nt, synthetic construct
  source 1..21: mol_type = other RNA
tattgaccttggtttcttaca

SEQ ID NO: 439 - RNA, 21 nt, synthetic construct
  source 1..21: mol_type = other RNA
tcatctacaagctggaaattc

SEQ ID NO: 440 - RNA, 21 nt, synthetic construct
  source 1..21: mol_type = other RNA
agtgaaggagacactattaaa

SEQ ID NO: 441 - RNA, 21 nt, synthetic construct
  source 1..21: mol_type = other RNA
atactagagatggggagtgga

SEQ ID NO: 442 - RNA, 21 nt, synthetic construct
  source 1..21: mol_type = other RNA
actcacctcactggttttagt

SEQ ID NO: 443 - RNA, 21 nt, synthetic construct
  source 1..21: mol_type = other RNA
gagatttcttaagtgacgcct

SEQ ID NO: 444 - RNA, 21 nt, synthetic construct
  source 1..21: mol_type = other RNA
ttataaacaacaggtgataca

SEQ ID NO: 445 - RNA, 21 nt, synthetic construct
  source 1..21: mol_type = other RNA
atctgaagaacagaagggcag

SEQ ID NO: 446 - RNA, 21 nt, synthetic construct
  source 1..21: mol_type = other RNA
cctaaaaacaagtagaaagct

SEQ ID NO: 447 - RNA, 21 nt, synthetic construct
  source 1..21: mol_type = other RNA
taaattaccaatacagaaagt

SEQ ID NO: 448 - RNA, 21 nt, synthetic construct
  source 1..21: mol_type = other RNA
agtggaaaatttgaactgatt

SEQ ID NO: 449 - RNA, 21 nt, synthetic construct
  source 1..21: mol_type = other RNA
cactgtttgttacaatatttt

SEQ ID NO: 450 - RNA, 21 nt, synthetic construct
  source 1..21: mol_type = other RNA
ttctcagtaaacagaaataac

SEQ ID NO: 451 - RNA, 21 nt, synthetic construct
  source 1..21: mol_type = other RNA
tatccctagtcttaaaaacaa

SEQ ID NO: 452 - RNA, 21 nt, synthetic construct
  source 1..21: mol_type = other RNA
tagtcatattcctttgattac

SEQ ID NO: 453 - RNA, 21 nt, synthetic construct
  source 1..21: mol_type = other RNA
ctaatttttttgttcttcatt

SEQ ID NO: 454 - RNA, 21 nt, synthetic construct
  source 1..21: mol_type = other RNA
aaataattattcttcatcata

SEQ ID NO: 455 - RNA, 21 nt, synthetic construct
  source 1..21: mol_type = other RNA
taaagtagaataaactcttta

SEQ ID NO: 456 - RNA, 21 nt, synthetic construct
  source 1..21: mol_type = other RNA
tctttgatagaaattaaaatc

SEQ ID NO: 457 - RNA, 21 nt, synthetic construct
  source 1..21: mol_type = other RNA
gaatactataactcaaattat

SEQ ID NO: 458 - RNA, 21 nt, synthetic construct
  source 1..21: mol_type = other RNA
cttattctgtgaggattacag

SEQ ID NO: 459 - RNA, 21 nt, synthetic construct
  source 1..21: mol_type = other RNA
ttacagggcactaaaaacaat

SEQ ID NO: 460 - RNA, 21 nt, synthetic construct
  source 1..21: mol_type = other RNA
ctcaaatatgttctactatag

SEQ ID NO: 461 - RNA, 21 nt, synthetic construct
  source 1..21: mol_type = other RNA
gaataagttcttatcttaatt

SEQ ID NO: 462 - RNA, 21 nt, synthetic construct
  source 1..21: mol_type = other RNA
aaagtgtaaagaataagatat

SEQ ID NO: 463 - RNA, 21 nt, synthetic construct
  source 1..21: mol_type = other RNA
aaaatttaatatactaaatgc

SEQ ID NO: 464 - RNA, 21 nt, synthetic construct
  source 1..21: mol_type = other RNA
taagaaaacaatttattttta

SEQ ID NO: 465 - RNA, 21 nt, synthetic construct
  source 1..21: mol_type = other RNA
taagaatatatatttgtttaa

SEQ ID NO: 466 - RNA, 21 nt, synthetic construct
  source 1..21: mol_type = other RNA
atgaatgttcttgaaaactca

SEQ ID NO: 467 - RNA, 21 nt, synthetic construct
  source 1..21: mol_type = other RNA
ccttttatattttaattggtt

SEQ ID NO: 468 - RNA, 21 nt, synthetic construct
  source 1..21: mol_type = other RNA
ttttaaaatgcttaatgttgc

SEQ ID NO: 469 - RNA, 21 nt, synthetic construct
  source 1..21: mol_type = other RNA
tcactatattaaaatgaatgt

SEQ ID NO: 470 - RNA, 21 nt, synthetic construct
  source 1..21: mol_type = other RNA
atgcaaatcagaatcactata

SEQ ID NO: 471 - RNA, 21 nt, synthetic construct
  source 1..21: mol_type = other RNA
tcatacaacacaatactctat

SEQ ID NO: 472 - RNA, 21 nt, synthetic construct
  source 1..21: mol_type = other RNA
tactgtgatgatcacagctgc

SEQ ID NO: 473 - RNA, 21 nt, synthetic construct
  source 1..21: mol_type = other RNA
agctaaacacccgtaagactt

SEQ ID NO: 474 - RNA, 21 nt, synthetic construct
  source 1..21: mol_type = other RNA
acagttatctcatatttggct

SEQ ID NO: 475 - RNA, 21 nt, synthetic construct
  source 1..21: mol_type = other RNA
ccaaggctacctaaaagaaga

SEQ ID NO: 476 - RNA, 21 nt, synthetic construct
  source 1..21: mol_type = other RNA
acagagtcagaaaactcccca

SEQ ID NO: 477 - RNA, 21 nt, synthetic construct
  source 1..21: mol_type = other RNA
aagagcctcaaccattgaaat

SEQ ID NO: 478 - RNA, 21 nt, synthetic construct
  source 1..21: mol_type = other RNA
ctcgaccacttaaaacttcag

SEQ ID NO: 479 - RNA, 21 nt, synthetic construct
  source 1..21: mol_type = other RNA
gacttcctgtcctgctggtat

SEQ ID NO: 480 - RNA, 21 nt, synthetic construct
  source 1..21: mol_type = other RNA
tgccagctttttatctttctc

SEQ ID NO: 481 - RNA, 21 nt, synthetic construct
  source 1..21: mol_type = other RNA
tcatggagaaagtccaatacc

SEQ ID NO: 482 - RNA, 21 nt, synthetic construct
  source 1..21: mol_type = other RNA
ctcactcgctcagctataaga

SEQ ID NO: 483 - RNA, 21 nt, synthetic construct
  source 1..21: mol_type = other RNA
caatttctgtctcatcttaat

SEQ ID NO: 484 - RNA, 21 nt, synthetic construct
  source 1..21: mol_type = other RNA
tatgtctcttgctgatctgta

SEQ ID NO: 485 - RNA, 21 nt, synthetic construct
  source 1..21: mol_type = other RNA
tgcctcaacaagcacgtcaaa

SEQ ID NO: 486 - RNA, 21 nt, synthetic construct
  source 1..21: mol_type = other RNA
aagttctgctacaacctctag

SEQ ID NO: 487 - RNA, 21 nt, synthetic construct
  source 1..21: mol_type = other RNA
atcatcgtgatgcttctctga

SEQ ID NO: 488 - RNA, 21 nt, synthetic construct
  source 1..21: mol_type = other RNA
aagctacagaatctatttatc

SEQ ID NO: 489 - RNA, 21 nt, synthetic construct
  source 1..21: mol_type = other RNA
tgagtagaagagtttctttgt

SEQ ID NO: 490 - RNA, 21 nt, synthetic construct
  source 1..21: mol_type = other RNA
gatctgcagcttgccacatca

SEQ ID NO: 491 - RNA, 21 nt, synthetic construct
  source 1..21: mol_type = other RNA
tgaaaaggtcaagattaagac

SEQ ID NO: 492 - RNA, 21 nt, synthetic construct
  source 1..21: mol_type = other RNA
atgcagacaggaaaacaatat

SEQ ID NO: 493 - RNA, 21 nt, synthetic construct
  source 1..21: mol_type = other RNA
ttgtataacagaccacttcct

SEQ ID NO: 494 - RNA, 21 nt, synthetic construct
  source 1..21: mol_type = other RNA
agcttaaaatctgtcatccca

SEQ ID NO: 495 - RNA, 21 nt, synthetic construct
  source 1..21: mol_type = other RNA
ctaaaacttattgttaccata

SEQ ID NO: 496 - RNA, 21 nt, synthetic construct
  source 1..21: mol_type = other RNA
caaaataagtgtataaaatgc

SEQ ID NO: 497 - RNA, 21 nt, synthetic construct
  source 1..21: mol_type = other RNA
atgtattcatctgttggatct

SEQ ID NO: 498 - RNA, 21 nt, synthetic construct
  source 1..21: mol_type = other RNA
ttgtaaacatgaaaagggctt

SEQ ID NO: 499 - RNA, 21 nt, synthetic construct
  source 1..21: mol_type = other RNA
ttattttcaaaaattaacttc

SEQ ID NO: 500 - RNA, 21 nt, synthetic construct
  source 1..21: mol_type = other RNA
caactgttgatttcctcaaca

SEQ ID NO: 501 - RNA, 21 nt, synthetic construct
  source 1..21: mol_type = other RNA
gcactgccaacaagttcactt

SEQ ID NO: 502 - RNA, 21 nt, synthetic construct
  source 1..21: mol_type = other RNA
aagagtttagttttaaaactg

SEQ ID NO: 503 - RNA, 21 nt, synthetic construct
  source 1..21: mol_type = other RNA
tcatatataaagcattatttt

SEQ ID NO: 504 - RNA, 21 nt, synthetic construct
  source 1..21: mol_type = other RNA
ttactcttttgaggtgaatat

SEQ ID NO: 505 - RNA, 21 nt, synthetic construct
  source 1..21: mol_type = other RNA
atggctcacaaatttctatcc

SEQ ID NO: 506 - RNA, 21 nt, synthetic construct
  source 1..21: mol_type = other RNA
ccaaatcttttctgaagatga

SEQ ID NO: 507 - RNA, 21 nt, synthetic construct
  source 1..21: mol_type = other RNA
aagcttctttaatactaagta

SEQ ID NO: 508 - RNA, 21 nt, synthetic construct
  source 1..21: mol_type = other RNA
atgaagtgtcattattcaaaa

SEQ ID NO: 509 - RNA, 21 nt, synthetic construct
  source 1..21: mol_type = other RNA
atagtccactgactcctcaca

SEQ ID NO: 510 - RNA, 21 nt, synthetic construct
  source 1..21: mol_type = other RNA
atttttcaggtcttcaccaag

SEQ ID NO: 511 - RNA, 21 nt, synthetic construct
  source 1..21: mol_type = other RNA
atctgttatcttattataaag

SEQ ID NO: 512 - RNA, 21 nt, synthetic construct
  source 1..21: mol_type = other RNA
taatttatattacaatgtaaa

SEQ ID NO: 513 - RNA, 21 nt, synthetic construct
  source 1..21: mol_type = other RNA
agaaattgtattttttctatg

SEQ ID NO: 514 - RNA, 21 nt, synthetic construct
  source 1..21: mol_type = other RNA
cagaatctacattctaaaaca

SEQ ID NO: 515 - RNA, 21 nt, synthetic construct
  source 1..21: mol_type = other RNA
gccacattaacatcttttaaa

SEQ ID NO: 516 - RNA, 21 nt, synthetic construct
  source 1..21: mol_type = other RNA
ggaaaagtaaggccatactct

SEQ ID NO: 517 - RNA, 21 nt, synthetic construct
  source 1..21: mol_type = other RNA
gaactatttgtagtaactatc

SEQ ID NO: 518 - RNA, 21 nt, synthetic construct
  source 1..21: mol_type = other RNA
ttacataataaaattcctttt

SEQ ID NO: 519 - RNA, 21 nt, synthetic construct
  source 1..21: mol_type = other RNA
taggtaaatcataaatctgtt

SEQ ID NO: 520 - RNA, 21 nt, synthetic construct
  source 1..21: mol_type = other RNA
gatgagaactggtggttaata

SEQ ID NO: 521 - RNA, 21 nt, synthetic construct
  source 1..21: mol_type = other RNA
taagtaattttttcaaagaat

SEQ ID NO: 522 - RNA, 21 nt, synthetic construct
  source 1..21: mol_type = other RNA
tctaagacatatgatcaacag

SEQ ID NO: 523 - RNA, 21 nt, synthetic construct
  source 1..21: mol_type = other RNA
atgtgacagtgagattagtca

SEQ ID NO: 524 - RNA, 21 nt, synthetic construct
  source 1..21: mol_type = other RNA
tcacagaattctagtacatgt

SEQ ID NO: 525 - RNA, 21 nt, synthetic construct
  source 1..21: mol_type = other RNA
aatcatgaaaccttaagactt

SEQ ID NO: 526 - RNA, 21 nt, synthetic construct
  source 1..21: mol_type = other RNA
aaggcactgtagtgaattatc

SEQ ID NO: 527 - RNA, 21 nt, synthetic construct
  source 1..21: mol_type = other RNA
acagaatctaatcttcattta

SEQ ID NO: 528 - RNA, 21 nt, synthetic construct
  source 1..21: mol_type = other RNA
ctgagctagagttacctagct

SEQ ID NO: 529 - RNA, 21 nt, synthetic construct
  source 1..21: mol_type = other RNA
agacttcagaatgattttgca

SEQ ID NO: 530 - RNA, 21 nt, synthetic construct
  source 1..21: mol_type = other RNA
atatcactaatatactaacaa

SEQ ID NO: 531 - RNA, 21 nt, synthetic construct
  source 1..21: mol_type = other RNA
atccaatgcaggcaaggaaaa

SEQ ID NO: 532 - RNA, 21 nt, synthetic construct
  source 1..21: mol_type = other RNA
tattttttaaaaatatatgaa

SEQ ID NO: 533 - RNA, 21 nt, synthetic construct
  source 1..21: mol_type = other RNA
gtcttccattccagcctaaca

SEQ ID NO: 534 - RNA, 21 nt, synthetic construct
  source 1..21: mol_type = other RNA
tcagaatgattttgcaggttg

SEQ ID NO: 535 - RNA, 21 nt, synthetic construct
  source 1..21: mol_type = other RNA
gaaaaatatattatctcaagt

SEQ ID NO: 536 - RNA, 21 nt, synthetic construct
  source 1..21: mol_type = other RNA
ataaaagatttccagtgacag

SEQ ID NO: 537 - RNA, 21 nt, synthetic construct
  source 1..21: mol_type = other RNA
tgaaatgaacttgttggccca

SEQ ID NO: 538 - RNA, 21 nt, synthetic construct
  source 1..21: mol_type = other RNA
attctctctccaaatattaac

SEQ ID NO: 539 - RNA, 21 nt, synthetic construct
  source 1..21: mol_type = other RNA
acccttgaacatgggggttag

SEQ ID NO: 540 - RNA, 21 nt, synthetic construct
  source 1..21: mol_type = other RNA
ctaattattagattatatttt

SEQ ID NO: 541 - RNA, 21 nt, synthetic construct
  source 1..21: mol_type = other RNA
ggttaggggagctgacaattc

SEQ ID NO: 542 - RNA, 21 nt, synthetic construct
  source 1..21: mol_type = other RNA
atctattacatctacagctga

SEQ ID NO: 543 - RNA, 21 nt, synthetic construct
  source 1..21: mol_type = other RNA
ttttgcgtgttatatgtatta

SEQ ID NO: 544 - RNA, 21 nt, synthetic construct
  source 1..21: mol_type = other RNA
aaacagtaaattaacacatat

SEQ ID NO: 545 - RNA, 21 nt, synthetic construct
  source 1..21: mol_type = other RNA
atacactatattcctacaata

SEQ ID NO: 546 - RNA, 21 nt, synthetic construct
  source 1..21: mol_type = other RNA
ataaaccttactgataacata

SEQ ID NO: 547 - RNA, 21 nt, synthetic construct
  source 1..21: mol_type = other RNA
ggggagctgacaattcgtggg

SEQ ID NO: 548 - RNA, 21 nt, synthetic construct
  source 1..21: mol_type = other RNA
ctaatagcctactattgacca

SEQ ID NO: 549 - RNA, 21 nt, synthetic construct
  source 1..21: mol_type = other RNA
caacataaaagtcttcattct

SEQ ID NO: 550 - RNA, 21 nt, synthetic construct
  source 1..21: mol_type = other RNA
aagagaaaatatatttactat

SEQ ID NO: 551 - RNA, 21 nt, synthetic construct
  source 1..21: mol_type = other RNA
ttatttagaaaatcataagaa

SEQ ID NO: 552 - RNA, 21 nt, synthetic construct
  source 1..21: mol_type = other RNA
ttcattaaatggaagtgggtc

SEQ ID NO: 553 - RNA, 21 nt, synthetic construct
  source 1..21: mol_type = other RNA
tcattgtcttcacattgagta

SEQ ID NO: 554 - RNA, 21 nt, synthetic construct
  source 1..21: mol_type = other RNA
aaagtaagctagagaaaatgt

SEQ ID NO: 555 - RNA, 21 nt, synthetic construct
  source 1..21: mol_type = other RNA
ggacccttgcaattcaagccc

SEQ ID NO: 556 - RNA, 21 nt, synthetic construct
  source 1..21: mol_type = other RNA
aggcagaggaggagaaagatg

SEQ ID NO: 557 - RNA, 21 nt, synthetic construct
  source 1..21: mol_type = other RNA
ggtcttgcagtcttgtcttag

SEQ ID NO: 558 - RNA, 21 nt, synthetic construct
  source 1..21: mol_type = other RNA
ggggtgtggggagtgggggaa

SEQ ID NO: 559 - RNA, 21 nt, synthetic construct
  source 1..21: mol_type = other RNA
aagaatattcatgtataagtg

SEQ ID NO: 560 - RNA, 21 nt, synthetic construct
  source 1..21: mol_type = other RNA
ggggaggaagagaaggcgttg

SEQ ID NO: 561 - RNA, 21 nt, synthetic construct
  source 1..21: mol_type = other RNA
cttgttgttcaagggtcaact

SEQ ID NO: 562 - RNA, 21 nt, synthetic construct
  source 1..21: mol_type = other RNA
agtccactctactgagctaaa

SEQ ID NO: 563 - RNA, 21 nt, synthetic construct
  source 1..21: mol_type = other RNA
attatagatccagctatgcta

SEQ ID NO: 564 - RNA, 21 nt, synthetic construct
  source 1..21: mol_type = other RNA
tgtaataggatatagctattt

SEQ ID NO: 565 - RNA, 21 nt, synthetic construct
  source 1..21: mol_type = other RNA
tttcttcctctatcaaccaaa

SEQ ID NO: 566 - RNA, 21 nt, synthetic construct
  source 1..21: mol_type = other RNA
atggtaagcatctattttgca

SEQ ID NO: 567 - RNA, 21 nt, synthetic construct
  source 1..21: mol_type = other RNA
caactgaagtaaaattgagtg

SEQ ID NO: 568 - RNA, 21 nt, synthetic construct
  source 1..21: mol_type = other RNA
cctctgaccatttcagaacat

SEQ ID NO: 569 - RNA, 21 nt, synthetic construct
  source 1..21: mol_type = other RNA
tcttccaataactcataaaac

SEQ ID NO: 570 - RNA, 21 nt, synthetic construct
  source 1..21: mol_type = other RNA
atttataattattttcttgat

SEQ ID NO: 571 - RNA, 21 nt, synthetic construct
  source 1..21: mol_type = other RNA
cttgatgaataaattttcaat

SEQ ID NO: 572 - RNA, 21 nt, synthetic construct
  source 1..21: mol_type = other RNA
tgaataaattttcaatttctc

SEQ ID NO: 573 - RNA, 21 nt, synthetic construct
  source 1..21: mol_type = other RNA
taatttcagttgaagacatgt

SEQ ID NO: 574 - RNA, 21 nt, synthetic construct
  source 1..21: mol_type = other RNA
actgcataatccaacaatttt

SEQ ID NO: 575 - RNA, 21 nt, synthetic construct
  source 1..21: mol_type = other RNA
aactttggtaatttaagttga

SEQ ID NO: 576 - RNA, 21 nt, synthetic construct
  source 1..21: mol_type = other RNA
actaaagtttaaaattaagtc

SEQ ID NO: 577 - RNA, 21 nt, synthetic construct
  source 1..21: mol_type = other RNA
ctaaaatagtttacacctata

SEQ ID NO: 578 - RNA, 21 nt, synthetic construct
  source 1..21: mol_type = other RNA
gctggaaaatatattcaccaa

SEQ ID NO: 579 - RNA, 21 nt, synthetic construct
  source 1..21: mol_type = other RNA
gtaacatcaatattgaccttg

SEQ ID NO: 580 - RNA, 21 nt, synthetic construct
  source 1..21: mol_type = other RNA
catggttactaaaagaatatg

SEQ ID NO: 581 - RNA, 21 nt, synthetic construct
  source 1..21: mol_type = other RNA
attaaccatatcttctaaaac

SEQ ID NO: 582 - RNA, 21 nt, synthetic construct
  source 1..21: mol_type = other RNA
acatgttactaatataactat

SEQ ID NO: 583 - RNA, 21 nt, synthetic construct
  source 1..21: mol_type = other RNA
aaaagagtagaggatgtgtaa

SEQ ID NO: 584 - RNA, 21 nt, synthetic construct
  source 1..21: mol_type = other RNA
ttactaatataactattatta

SEQ ID NO: 585 - RNA, 21 nt, synthetic construct
  source 1..21: mol_type = other RNA
cccatgagagatactagagat

SEQ ID NO: 586 - RNA, 21 nt, synthetic construct
  source 1..21: mol_type = other RNA
ctgaacccatgagagatacta

SEQ ID NO: 587 - RNA, 21 nt, synthetic construct
  source 1..21: mol_type = other RNA
ctaactcaatagtgaaggaga

SEQ ID NO: 588 - RNA, 21 nt, synthetic construct
  source 1..21: mol_type = other RNA
tggggagtggaaagtgtttgg

SEQ ID NO: 589 - RNA, 21 nt, synthetic construct
  source 1..21: mol_type = other RNA
acactattaaattttctgaac

SEQ ID NO: 590 - RNA, 21 nt, synthetic construct
  source 1..21: mol_type = other RNA
ggtttcttacacaagtgttgc

SEQ ID NO: 591 - RNA, 21 nt, synthetic construct
  source 1..21: mol_type = other RNA
gctggaaattcctaaaaacaa

SEQ ID NO: 592 - RNA, 21 nt, synthetic construct
  source 1..21: mol_type = other RNA
aagtgacgcctcatctacaag

SEQ ID NO: 593 - RNA, 21 nt, synthetic construct
  source 1..21: mol_type = other RNA
cagaagggcagagatttctta

SEQ ID NO: 594 - RNA, 21 nt, synthetic construct
  source 1..21: mol_type = other RNA
tacaagctggaaattcctaaa

SEQ ID NO: 595 - RNA, 21 nt, synthetic construct
  source 1..21: mol_type = other RNA
gttcagggatatctgaagaac

SEQ ID NO: 596 - RNA, 21 nt, synthetic construct
  source 1..21: mol_type = other RNA
agtagaaagcttataaacaac

SEQ ID NO: 597 - RNA, 21 nt, synthetic construct
  source 1..21: mol_type = other RNA
ttgaactgattagtcatattc

SEQ ID NO: 598 - RNA, 21 nt, synthetic construct
  source 1..21: mol_type = other RNA
atacagaaagtatccctagtc

SEQ ID NO: 599 - RNA, 21 nt, synthetic construct
  source 1..21: mol_type = other RNA
atattcctttgattacactgt

SEQ ID NO: 600 - RNA, 21 nt, synthetic construct
  source 1..21: mol_type = other RNA
ctggttttagtaaattaccaa

SEQ ID NO: 601 - RNA, 21 nt, synthetic construct
  source 1..21: mol_type = other RNA
caggtgatacactcacctcac

SEQ ID NO: 602 - RNA, 21 nt, synthetic construct
  source 1..21: mol_type = other RNA
cttaaaaacaagtggaaaatt

SEQ ID NO: 603 - RNA, 21 nt, synthetic construct
  source 1..21: mol_type = other RNA
tacaatatttttctcagtaaa

SEQ ID NO: 604 - RNA, 21 nt, synthetic construct
  source 1..21: mol_type = other RNA
cctttgattacactgtttgtt

SEQ ID NO: 605 - RNA, 21 nt, synthetic construct
  source 1..21: mol_type = other RNA
tgttcttcattctttgataga

SEQ ID NO: 606 - RNA, 21 nt, synthetic construct
  source 1..21: mol_type = other RNA
gatagaaattaaaatcttatt

SEQ ID NO: 607 - RNA, 21 nt, synthetic construct
  source 1..21: mol_type = other RNA
acagaaataactaattttttt

SEQ ID NO: 608 - RNA, 21 nt, synthetic construct
  source 1..21: mol_type = other RNA
aaattaaaatcttattctgtg

SEQ ID NO: 609 - RNA, 21 nt, synthetic construct
  source 1..21: mol_type = other RNA
tagaataaactctttaaataa

SEQ ID NO: 610 - RNA, 21 nt, synthetic construct
  source 1..21: mol_type = other RNA
gaggattacagaatactataa

SEQ ID NO: 611 - RNA, 21 nt, synthetic construct
  source 1..21: mol_type = other RNA
gaataagatataagaaaacaa

SEQ ID NO: 612 - RNA, 21 nt, synthetic construct
  source 1..21: mol_type = other RNA
taaactctttaaataattatt

SEQ ID NO: 613 - RNA, 21 nt, synthetic construct
  source 1..21: mol_type = other RNA
actcaaattataaagtagaat

SEQ ID NO: 614 - RNA, 21 nt, synthetic construct
  source 1..21: mol_type = other RNA
tcttcatcataaagtgtaaag

SEQ ID NO: 615 - RNA, 21 nt, synthetic construct
  source 1..21: mol_type = other RNA
ctaaaaacaattttaaaatgc

SEQ ID NO: 616 - RNA, 21 nt, synthetic construct
  source 1..21: mol_type = other RNA
ttctactatagaataagttct

SEQ ID NO: 617 - RNA, 21 nt, synthetic construct
  source 1..21: mol_type = other RNA
agttcttatcttaatttacag

SEQ ID NO: 618 - RNA, 21 nt, synthetic construct
  source 1..21: mol_type = other RNA
ttatcttaatttacagggcac

SEQ ID NO: 619 - RNA, 21 nt, synthetic construct
  source 1..21: mol_type = other RNA
atactaaatgctcaaatatgt

SEQ ID NO: 620 - RNA, 21 nt, synthetic construct
  source 1..21: mol_type = other RNA
atttatttttaaaatttaata

SEQ ID NO: 621 - RNA, 21 nt, synthetic construct
  source 1..21: mol_type = other RNA
cttaatgttgccttttatatt

SEQ ID NO: 622 - RNA, 21 nt, synthetic construct
  source 1..21: mol_type = other RNA
attaaaatgaatgttcttgaa

SEQ ID NO: 623 - RNA, 21 nt, synthetic construct
  source 1..21: mol_type = other RNA
tttaattggttaagaatatat

SEQ ID NO: 624 - RNA, 21 nt, synthetic construct
  source 1..21: mol_type = other RNA
tatttgtttaatgcaaatcag

SEQ ID NO: 625 - RNA, 21 nt, synthetic construct
  source 1..21: mol_type = other RNA
tcagaatcactatattaaaat

SEQ ID NO: 626 - RNA, 21 nt, synthetic construct
  source 1..21: mol_type = other RNA
aaatgcttaatgttgcctttt

SEQ ID NO: 627 - RNA, 23 nt, synthetic construct
  source 1..23: mol_type = other RNA
tagattctgtagcttttgacgtg

SEQ ID NO: 628 - RNA, 23 nt, synthetic construct
  source 1..23: mol_type = other RNA
taaatagattctgtagcttttga

SEQ ID NO: 629 - RNA, 23 nt, synthetic construct
  source 1..23: mol_type = other RNA
tgataaatagattctgtagcttt

SEQ ID NO: 630 - RNA, 23 nt, synthetic construct
  source 1..23: mol_type = other RNA
ttgataaatagattctgtagctt

SEQ ID NO: 631 - RNA, 23 nt, synthetic construct
  source 1..23: mol_type = other RNA
tgagacagaaattgataaataga

SEQ ID NO: 632 - RNA, 23 nt, synthetic construct
  source 1..23: mol_type = other RNA
taagatgagacagaaattgataa

SEQ ID NO: 633 - RNA, 23 nt, synthetic construct
  source 1..23: mol_type = other RNA
ttaagatgagacagaaattgata

SEQ ID NO: 634 - RNA, 23 nt, synthetic construct
  source 1..23: mol_type = other RNA
tattaagatgagacagaaattga

SEQ ID NO: 635 - RNA, 23 nt, synthetic construct
  source 1..23: mol_type = other RNA
tagtcttaatcttgaccttttca

SEQ ID NO: 636 - RNA, 23 nt, synthetic construct
  source 1..23: mol_type = other RNA
ttagtcttaatcttgaccttttc

SEQ ID NO: 637 - RNA, 23 nt, synthetic construct
  source 1..23: mol_type = other RNA
tttagtcttaatcttgacctttt

SEQ ID NO: 638 - RNA, 23 nt, synthetic construct
  source 1..23: mol_type = other RNA
ttttagtcttaatcttgaccttt

SEQ ID NO: 639 - RNA, 23 nt, synthetic construct
  source 1..23: mol_type = other RNA
taagttttagtcttaatcttgac

SEQ ID NO: 640 - RNA, 23 nt, synthetic construct
  source 1..23: mol_type = other RNA
taacaataagttttagtcttaat

SEQ ID NO: 641 - RNA, 23 nt, synthetic construct
  source 1..23: mol_type = other RNA
tggtaacaataagttttagtctt

SEQ ID NO: 642 - RNA, 23 nt, synthetic construct
  source 1..23: mol_type = other RNA
tatggtaacaataagttttagtc

SEQ ID NO: 643 - RNA, 23 nt, synthetic construct
  source 1..23: mol_type = other RNA
tccaacagatgaatacatatggt

SEQ ID NO: 644 - RNA, 23 nt, synthetic construct
  source 1..23: mol_type = other RNA
tacaagatccaacagatgaatac

SEQ ID NO: 645 - RNA, 23 nt, synthetic construct
  source 1..23: mol_type = other RNA
ttacaagatccaacagatgaata

SEQ ID NO: 646 - RNA, 23 nt, synthetic construct
  source 1..23: mol_type = other RNA
tttacaagatccaacagatgaat

SEQ ID NO: 647 - RNA, 23 nt, synthetic construct
  source 1..23: mol_type = other RNA
tgtttacaagatccaacagatga

SEQ ID NO: 648 - RNA, 23 nt, synthetic construct
  source 1..23: mol_type = other RNA
tcatgtttacaagatccaacaga

SEQ ID NO: 649 - RNA, 23 nt, synthetic construct
  source 1..23: mol_type = other RNA
ttcatgtttacaagatccaacag

SEQ ID NO: 650 - RNA, 23 nt, synthetic construct
  source 1..23: mol_type = other RNA
tttcatgtttacaagatccaaca

SEQ ID NO: 651 - RNA, 23 nt, synthetic construct
  source 1..23: mol_type = other RNA
ttttcatgtttacaagatccaac

SEQ ID NO: 652 - RNA, 23 nt, synthetic construct
  source 1..23: mol_type = other RNA
tacacttattttgaagttaattt

SEQ ID NO: 653 - RNA, 23 nt, synthetic construct
  source 1..23: mol_type = other RNA
tatacacttattttgaagttaat

SEQ ID NO: 654 - RNA, 23 nt, synthetic construct
  source 1..23: mol_type = other RNA
ttatacacttattttgaagttaa

SEQ ID NO: 655 - RNA, 23 nt, synthetic construct
  source 1..23: mol_type = other RNA
tttatacacttattttgaagtta

SEQ ID NO: 656 - RNA, 23 nt, synthetic construct
  source 1..23: mol_type = other RNA
ttttatacacttattttgaagtt

SEQ ID NO: 657 - RNA, 23 nt, synthetic construct
  source 1..23: mol_type = other RNA
tgcattttatacacttattttga

SEQ ID NO: 658 - RNA, 23 nt, synthetic construct
  source 1..23: mol_type = other RNA
ttgcattttatacacttattttg

SEQ ID NO: 659 - RNA, 23 nt, synthetic construct
  source 1..23: mol_type = other RNA
tcaacagttgcattttatacact

SEQ ID NO: 660 - RNA, 23 nt, synthetic construct
  source 1..23: mol_type = other RNA
ttcatcttcagaaaagatttggg

SEQ ID NO: 661 - RNA, 23 nt, synthetic construct
  source 1..23: mol_type = other RNA
tcttcatcttcagaaaagatttg

SEQ ID NO: 662 - RNA, 23 nt, synthetic construct
  source 1..23: mol_type = other RNA
taaactcttcatcttcagaaaag

SEQ ID NO: 663 - RNA, 23 nt, synthetic construct
  source 1..23: mol_type = other RNA
taaaactaaactcttcatcttca

SEQ ID NO: 664 - RNA, 23 nt, synthetic construct
  source 1..23: mol_type = other RNA
ttaaaactaaactcttcatcttc

SEQ ID NO: 665 - RNA, 23 nt, synthetic construct
  source 1..23: mol_type = other RNA
tttaaaactaaactcttcatctt

SEQ ID NO: 666 - RNA, 23 nt, synthetic construct
  source 1..23: mol_type = other RNA
ttttaaaactaaactcttcatct

SEQ ID NO: 667 - RNA, 23 nt, synthetic construct
  source 1..23: mol_type = other RNA
tgcagttttaaaactaaactctt

SEQ ID NO: 668 - RNA, 23 nt, synthetic construct
  source 1..23: mol_type = other RNA
tggcagtgcagttttaaaactaa

SEQ ID NO: 669 - RNA, 23 nt, synthetic construct
  source 1..23: mol_type = other RNA
ttggcagtgcagttttaaaacta

SEQ ID NO: 670 - RNA, 23 nt, synthetic construct
  source 1..23: mol_type = other RNA
tatattttctctttcttatgatt

SEQ ID NO: 671 - RNA, 23 nt, synthetic construct
  source 1..23: mol_type = other RNA
taaatatattttctctttcttat

SEQ ID NO: 672 - RNA, 23 nt, synthetic construct
  source 1..23: mol_type = other RNA
tagtaaatatattttctctttct

SEQ ID NO: 673 - RNA, 23 nt, synthetic construct
  source 1..23: mol_type = other RNA
tgaatagtaaatatattttctct

SEQ ID NO: 674 - RNA, 23 nt, synthetic construct
  source 1..23: mol_type = other RNA
taatgaatagtaaatatattttc

SEQ ID NO: 675 - RNA, 23 nt, synthetic construct
  source 1..23: mol_type = other RNA
ttaatgaatagtaaatatatttt

SEQ ID NO: 676 - RNA, 23 nt, synthetic construct
  source 1..23: mol_type = other RNA
tttaatgaatagtaaatatattt

SEQ ID NO: 677 - RNA, 23 nt, synthetic construct
  source 1..23: mol_type = other RNA
tttctgtagcttttgacgtgctt

SEQ ID NO: 678 - RNA, 23 nt, synthetic construct
  source 1..23: mol_type = other RNA
tattctgtagcttttgacgtgct

SEQ ID NO: 679 - RNA, 23 nt, synthetic construct
  source 1..23: mol_type = other RNA
tgattctgtagcttttgacgtgc

SEQ ID NO: 680 - RNA, 23 nt, synthetic construct
  source 1..23: mol_type = other RNA
ttagattctgtagcttttgacgt

SEQ ID NO: 681 - RNA, 23 nt, synthetic construct
  source 1..23: mol_type = other RNA
tatagattctgtagcttttgacg

SEQ ID NO: 682 - RNA, 23 nt, synthetic construct
  source 1..23: mol_type = other RNA
taatagattctgtagcttttgac

SEQ ID NO: 683 - RNA, 23 nt, synthetic construct
  source 1..23: mol_type = other RNA
ttaaatagattctgtagcttttg

SEQ ID NO: 684 - RNA, 23 nt, synthetic construct
  source 1..23: mol_type = other RNA
tataaatagattctgtagctttt

SEQ ID NO: 685 - RNA, 23 nt, synthetic construct
  source 1..23: mol_type = other RNA
tttgataaatagattctgtagct

SEQ ID NO: 686 - RNA, 23 nt, synthetic construct
  source 1..23: mol_type = other RNA
tattgataaatagattctgtagc

SEQ ID NO: 687 - RNA, 23 nt, synthetic construct
  source 1..23: mol_type = other RNA
taattgataaatagattctgtag

SEQ ID NO: 688 - RNA, 23 nt, synthetic construct
  source 1..23: mol_type = other RNA
taaattgataaatagattctgta

SEQ ID NO: 689 - RNA, 23 nt, synthetic construct
  source 1..23: mol_type = other RNA
tgaaattgataaatagattctgt

SEQ ID NO: 690 - RNA, 23 nt, synthetic construct
  source 1..23: mol_type = other RNA
tagaaattgataaatagattctg

SEQ ID NO: 691 - RNA, 23 nt, synthetic construct
  source 1..23: mol_type = other RNA
tcagaaattgataaatagattct

SEQ ID NO: 692 - RNA, 23 nt, synthetic construct
  source 1..23: mol_type = other RNA
tacagaaattgataaatagattc

SEQ ID NO: 693 - RNA, 23 nt, synthetic construct
  source 1..23: mol_type = other RNA
tgacagaaattgataaatagatt

SEQ ID NO: 694 - RNA, 23 nt, synthetic construct
  source 1..23: mol_type = other RNA
tagacagaaattgataaatagat

SEQ ID NO: 695 - RNA, 23 nt, synthetic construct
  source 1..23: mol_type = other RNA
ttgagacagaaattgataaatag

SEQ ID NO: 696 - RNA, 23 nt, synthetic construct
  source 1..23: mol_type = other RNA
tatgagacagaaattgataaata

SEQ ID NO: 697 - RNA, 23 nt, synthetic construct
  source 1..23: mol_type = other RNA
tgatgagacagaaattgataaat

SEQ ID NO: 698 - RNA, 23 nt, synthetic construct
  source 1..23: mol_type = other RNA
tagatgagacagaaattgataaa

SEQ ID NO: 699 - RNA, 23 nt, synthetic construct
  source 1..23: mol_type = other RNA
tttaagatgagacagaaattgat

SEQ ID NO: 700 - RNA, 23 nt, synthetic construct
  source 1..23: mol_type = other RNA
ttattaagatgagacagaaattg

SEQ ID NO: 701 - RNA, 23 nt, synthetic construct
  source 1..23: mol_type = other RNA
tatattaagatgagacagaaatt

SEQ ID NO: 702 - RNA, 23 nt, synthetic construct
  source 1..23: mol_type = other RNA
tcatattaagatgagacagaaat

SEQ ID NO: 703 - RNA, 23 nt, synthetic construct
  source 1..23: mol_type = other RNA
tacatattaagatgagacagaaa

SEQ ID NO: 704 - RNA, 23 nt, synthetic construct
  source 1..23: mol_type = other RNA
tgacatattaagatgagacagaa

SEQ ID NO: 705 - RNA, 23 nt, synthetic construct
  source 1..23: mol_type = other RNA
tagacatattaagatgagacaga

SEQ ID NO: 706 - RNA, 23 nt, synthetic construct
  source 1..23: mol_type = other RNA
tgagacatattaagatgagacag

SEQ ID NO: 707 - RNA, 23 nt, synthetic construct
  source 1..23: mol_type = other RNA
tagagacatattaagatgagaca

SEQ ID NO: 708 - RNA, 23 nt, synthetic construct
  source 1..23: mol_type = other RNA
taagagacatattaagatgagac

SEQ ID NO: 709 - RNA, 23 nt, synthetic construct
  source 1..23: mol_type = other RNA
tgtcttaatcttgaccttttcac

SEQ ID NO: 710 - RNA, 23 nt, synthetic construct
  source 1..23: mol_type = other RNA
tttttagtcttaatcttgacctt

SEQ ID NO: 711 - RNA, 23 nt, synthetic construct
  source 1..23: mol_type = other RNA
tgttttagtcttaatcttgacct

SEQ ID NO: 712 - RNA, 23 nt, synthetic construct
  source 1..23: mol_type = other RNA
tagttttagtcttaatcttgacc

SEQ ID NO: 713 - RNA, 23 nt, synthetic construct
  source 1..23: mol_type = other RNA
ttaagttttagtcttaatcttga

SEQ ID NO: 714 - RNA, 23 nt, synthetic construct
  source 1..23: mol_type = other RNA
tataagttttagtcttaatcttg

SEQ ID NO: 715 - RNA, 23 nt, synthetic construct
  source 1..23: mol_type = other RNA
taataagttttagtcttaatctt

SEQ ID NO: 716 - RNA, 23 nt, synthetic construct
  source 1..23: mol_type = other RNA
tcaataagttttagtcttaatct

SEQ ID NO: 717 - RNA, 23 nt, synthetic construct
  source 1..23: mol_type = other RNA
tacaataagttttagtcttaatc

SEQ ID NO: 718 - RNA, 23 nt, synthetic construct
  source 1..23: mol_type = other RNA
ttaacaataagttttagtcttaa

SEQ ID NO: 719 - RNA, 23 nt, synthetic construct
  source 1..23: mol_type = other RNA
tgtaacaataagttttagtctta

SEQ ID NO: 720 - RNA, 23 nt, synthetic construct
  source 1..23: mol_type = other RNA
ttggtaacaataagttttagtct

SEQ ID NO: 721 - RNA, 23 nt, synthetic construct
  source 1..23: mol_type = other RNA
tgatgaatacatatggtaacaat

SEQ ID NO: 722 - RNA, 23 nt, synthetic construct
  source 1..23: mol_type = other RNA
tagatgaatacatatggtaacaa

SEQ ID NO: 723 - RNA, 23 nt, synthetic construct
  source 1..23: mol_type = other RNA
tcagatgaatacatatggtaaca

SEQ ID NO: 724 - RNA, 23 nt, synthetic construct
  source 1..23: mol_type = other RNA
tacagatgaatacatatggtaac

SEQ ID NO: 725 - RNA, 23 nt, synthetic construct
  source 1..23: mol_type = other RNA
taacagatgaatacatatggtaa

SEQ ID NO: 726 - RNA, 23 nt, synthetic construct
  source 1..23: mol_type = other RNA
tcaacagatgaatacatatggta

SEQ ID NO: 727 - RNA, 23 nt, synthetic construct
  source 1..23: mol_type = other RNA
ttccaacagatgaatacatatgg

SEQ ID NO: 728 - RNA, 23 nt, synthetic construct
  source 1..23: mol_type = other RNA
tatccaacagatgaatacatatg

SEQ ID NO: 729 - RNA, 23 nt, synthetic construct
  source 1..23: mol_type = other RNA
tgatccaacagatgaatacatat

SEQ ID NO: 730 - RNA, 23 nt, synthetic construct
  source 1..23: mol_type = other RNA
tagatccaacagatgaatacata

SEQ ID NO: 731 - RNA, 23 nt, synthetic construct
  source 1..23: mol_type = other RNA
taagatccaacagatgaatacat

SEQ ID NO: 732 - RNA, 23 nt, synthetic construct
  source 1..23: mol_type = other RNA
tcaagatccaacagatgaataca

SEQ ID NO: 733 - RNA, 23 nt, synthetic construct
  source 1..23: mol_type = other RNA
ttttacaagatccaacagatgaa

SEQ ID NO: 734 - RNA, 23 nt, synthetic construct
  source 1..23: mol_type = other RNA
ttgtttacaagatccaacagatg

SEQ ID NO: 735 - RNA, 23 nt, synthetic construct
  source 1..23: mol_type = other RNA
tatgtttacaagatccaacagat

SEQ ID NO: 736 - RNA, 23 nt, synthetic construct
  source 1..23: mol_type = other RNA
tttttcatgtttacaagatccaa

SEQ ID NO: 737 - RNA, 23 nt, synthetic construct
  source 1..23: mol_type = other RNA
tcttttcatgtttacaagatcca

SEQ ID NO: 738 - RNA, 23 nt, synthetic construct
  source 1..23: mol_type = other RNA
tccttttcatgtttacaagatcc

SEQ ID NO: 739 - RNA, 23 nt, synthetic construct
  source 1..23: mol_type = other RNA
tcccttttcatgtttacaagatc

SEQ ID NO: 740 - RNA, 23 nt, synthetic construct
  source 1..23: mol_type = other RNA
tgcccttttcatgtttacaagat

SEQ ID NO: 741 - RNA, 23 nt, synthetic construct
  source 1..23: mol_type = other RNA
tcacttattttgaagttaatttt

SEQ ID NO: 742 - RNA, 23 nt, synthetic construct
  source 1..23: mol_type = other RNA
ttacacttattttgaagttaatt

SEQ ID NO: 743 - RNA, 23 nt, synthetic construct
  source 1..23: mol_type = other RNA
tttttatacacttattttgaagt

SEQ ID NO: 744 - RNA, 23 nt, synthetic construct
  source 1..23: mol_type = other RNA
tattttatacacttattttgaag

SEQ ID NO: 745 - RNA, 23 nt, synthetic construct
  source 1..23: mol_type = other RNA
tcattttatacacttattttgaa

SEQ ID NO: 746 - RNA, 23 nt, synthetic construct
  source 1..23: mol_type = other RNA
tttgcattttatacacttatttt

SEQ ID NO: 747 - RNA, 23 nt, synthetic construct
  source 1..23: mol_type = other RNA
tgttgcattttatacacttattt

SEQ ID NO: 748 - RNA, 23 nt, synthetic construct
  source 1..23: mol_type = other RNA
tagttgcattttatacacttatt

SEQ ID NO: 749 - RNA, 23 nt, synthetic construct
  source 1..23: mol_type = other RNA
tcagttgcattttatacacttat

SEQ ID NO: 750 - RNA, 23 nt, synthetic construct
  source 1..23: mol_type = other RNA
tacagttgcattttatacactta

SEQ ID NO: 751 - RNA, 23 nt, synthetic construct
  source 1..23: mol_type = other RNA
taacagttgcattttatacactt

SEQ ID NO: 752 - RNA, 23 nt, synthetic construct
  source 1..23: mol_type = other RNA
tttcatcttcagaaaagatttgg

SEQ ID NO: 753 - RNA, 23 nt, synthetic construct
  source 1..23: mol_type = other RNA
ttcttcatcttcagaaaagattt

SEQ ID NO: 754 - RNA, 23 nt, synthetic construct
  source 1..23: mol_type = other RNA
tctcttcatcttcagaaaagatt

SEQ ID NO: 755 - RNA, 23 nt, synthetic construct
  source 1..23: mol_type = other RNA
tactcttcatcttcagaaaagat

SEQ ID NO: 756 - RNA, 23 nt, synthetic construct
  source 1..23: mol_type = other RNA
taactcttcatcttcagaaaaga

SEQ ID NO: 757 - RNA, 23 nt, synthetic construct
  source 1..23: mol_type = other RNA
ttaaactcttcatcttcagaaaa

SEQ ID NO: 758 - RNA, 23 nt, synthetic construct
  source 1..23: mol_type = other RNA
tctaaactcttcatcttcagaaa

SEQ ID NO: 759 - RNA, 23 nt, synthetic construct
  source 1..23: mol_type = other RNA
tactaaactcttcatcttcagaa

SEQ ID NO: 760 - RNA, 23 nt, synthetic construct
  source 1..23: mol_type = other RNA
taactaaactcttcatcttcaga

SEQ ID NO: 761 - RNA, 23 nt, synthetic construct
  source 1..23: mol_type = other RNA
taaactaaactcttcatcttcag

SEQ ID NO: 762 - RNA, 23 nt, synthetic construct
  source 1..23: mol_type = other RNA
tttttaaaactaaactcttcatc

SEQ ID NO: 763 - RNA, 23 nt, synthetic construct
  source 1..23: mol_type = other RNA
tgttttaaaactaaactcttcat

SEQ ID NO: 764 - RNA, 23 nt, synthetic construct
  source 1..23: mol_type = other RNA
tagttttaaaactaaactcttca

SEQ ID NO: 765 - RNA, 23 nt, synthetic construct
  source 1..23: mol_type = other RNA
tcagttttaaaactaaactcttc

SEQ ID NO: 766 - RNA, 23 nt, synthetic construct
  source 1..23: mol_type = other RNA
ttgcagttttaaaactaaactct

SEQ ID NO: 767 - RNA, 23 nt, synthetic construct
  source 1..23: mol_type = other RNA
tgtgcagttttaaaactaaactc

SEQ ID NO: 768 - RNA, 23 nt, synthetic construct
  source 1..23: mol_type = other RNA
tagtgcagttttaaaactaaact

SEQ ID NO: 769 - RNA, 23 nt, synthetic construct
  source 1..23: mol_type = other RNA
tcagtgcagttttaaaactaaac

SEQ ID NO: 770 - RNA, 23 nt, synthetic construct
  source 1..23: mol_type = other RNA
tgcagtgcagttttaaaactaaa

SEQ ID NO: 771 - RNA, 23 nt, synthetic construct
  source 1..23: mol_type = other RNA
ttattttctctttcttatgattt

SEQ ID NO: 772 - RNA, 23 nt, synthetic construct
  source 1..23: mol_type = other RNA
ttatattttctctttcttatgat

SEQ ID NO: 773 - RNA, 23 nt, synthetic construct
  source 1..23: mol_type = other RNA
tatatattttctctttcttatga

SEQ ID NO: 774 - RNA, 23 nt, synthetic construct
  source 1..23: mol_type = other RNA
taatatattttctctttcttatg

SEQ ID NO: 775 - RNA, 23 nt, synthetic construct
  source 1..23: mol_type = other RNA
ttaaatatattttctctttctta

SEQ ID NO: 776 - RNA, 23 nt, synthetic construct
  source 1..23: mol_type = other RNA
tgtaaatatattttctctttctt

SEQ ID NO: 777 - RNA, 23 nt, synthetic construct
  source 1..23: mol_type = other RNA
ttagtaaatatattttctctttc

SEQ ID NO: 778 - RNA, 23 nt, synthetic construct
  source 1..23: mol_type = other RNA
tatagtaaatatattttctcttt

SEQ ID NO: 779 - RNA, 23 nt, synthetic construct
  source 1..23: mol_type = other RNA
taatagtaaatatattttctctt

SEQ ID NO: 780 - RNA, 23 nt, synthetic construct
  source 1..23: mol_type = other RNA
ttgaatagtaaatatattttctc

SEQ ID NO: 781 - RNA, 23 nt, synthetic construct
  source 1..23: mol_type = other RNA
tatgaatagtaaatatattttct

SEQ ID NO: 782 - RNA, 23 nt, synthetic construct
  source 1..23: mol_type = other RNA
ttttaatgaatagtaaatatatt

SEQ ID NO: 783 - RNA, 21 nt, synthetic construct
  source 1..21: mol_type = other RNA
cgtcaaaagctacagaatcta

SEQ ID NO: 784 - RNA, 21 nt, synthetic construct
  source 1..21: mol_type = other RNA
aaaagctacagaatctattta

SEQ ID NO: 785 - RNA, 21 nt, synthetic construct
  source 1..21: mol_type = other RNA
agctacagaatctatttatca

SEQ ID NO: 786 - RNA, 21 nt, synthetic construct
  source 1..21: mol_type = other RNA
gctacagaatctatttatcaa

SEQ ID NO: 787 - RNA, 21 nt, synthetic construct
  source 1..21: mol_type = other RNA
tatttatcaatttctgtctca

SEQ ID NO: 788 - RNA, 21 nt, synthetic construct
  source 1..21: mol_type = other RNA
atcaatttctgtctcatctta

SEQ ID NO: 789 - RNA, 21 nt, synthetic construct
  source 1..21: mol_type = other RNA
tcaatttctgtctcatcttaa

SEQ ID NO: 790 - RNA, 21 nt, synthetic construct
  source 1..21: mol_type = other RNA
aatttctgtctcatcttaata

SEQ ID NO: 791 - RNA, 21 nt, synthetic construct
  source 1..21: mol_type = other RNA
aaaaggtcaagattaagacta

SEQ ID NO: 792 - RNA, 21 nt, synthetic construct
  source 1..21: mol_type = other RNA
aaaggtcaagattaagactaa

SEQ ID NO: 793 - RNA, 21 nt, synthetic construct
  source 1..21: mol_type = other RNA
aaggtcaagattaagactaaa

SEQ ID NO: 794 - RNA, 21 nt, synthetic construct
  source 1..21: mol_type = other RNA
aggtcaagattaagactaaaa

SEQ ID NO: 795 - RNA, 21 nt, synthetic construct
  source 1..21: mol_type = other RNA
caagattaagactaaaactta

SEQ ID NO: 796 - RNA, 21 nt, synthetic construct
  source 1..21: mol_type = other RNA
taagactaaaacttattgtta

SEQ ID NO: 797 - RNA, 21 nt, synthetic construct
  source 1..21: mol_type = other RNA
gactaaaacttattgttacca

SEQ ID NO: 798 - RNA, 21 nt, synthetic construct
  source 1..21: mol_type = other RNA
ctaaaacttattgttaccata

SEQ ID NO: 799 - RNA, 21 nt, synthetic construct
  source 1..21: mol_type = other RNA
catatgtattcatctgttgga

SEQ ID NO: 800 - RNA, 21 nt, synthetic construct
  source 1..21: mol_type = other RNA
attcatctgttggatcttgta

SEQ ID NO: 801 - RNA, 21 nt, synthetic construct
  source 1..21: mol_type = other RNA
ttcatctgttggatcttgtaa

SEQ ID NO: 802 - RNA, 21 nt, synthetic construct
  source 1..21: mol_type = other RNA
tcatctgttggatcttgtaaa

SEQ ID NO: 803 - RNA, 21 nt, synthetic construct
  source 1..21: mol_type = other RNA
atctgttggatcttgtaaaca

SEQ ID NO: 804 - RNA, 21 nt, synthetic construct
  source 1..21: mol_type = other RNA
tgttggatcttgtaaacatga

SEQ ID NO: 805 - RNA, 21 nt, synthetic construct
  source 1..21: mol_type = other RNA
gttggatcttgtaaacatgaa

SEQ ID NO: 806 - RNA, 21 nt, synthetic construct
  source 1..21: mol_type = other RNA
ttggatcttgtaaacatgaaa

SEQ ID NO: 807 - RNA, 21 nt, synthetic construct
  source 1..21: mol_type = other RNA
tggatcttgtaaacatgaaaa

SEQ ID NO: 808 - RNA, 21 nt, synthetic construct
  source 1..21: mol_type = other RNA
attaacttcaaaataagtgta

SEQ ID NO: 809 - RNA, 21 nt, synthetic construct
  source 1..21: mol_type = other RNA
taacttcaaaataagtgtata

SEQ ID NO: 810 - RNA, 21 nt, synthetic construct
  source 1..21: mol_type = other RNA
aacttcaaaataagtgtataa

SEQ ID NO: 811 - RNA, 21 nt, synthetic construct
  source 1..21: mol_type = other RNA
acttcaaaataagtgtataaa

SEQ ID NO: 812 - RNA, 21 nt, synthetic construct
  source 1..21: mol_type = other RNA
cttcaaaataagtgtataaaa

SEQ ID NO: 813 - RNA, 21 nt, synthetic construct
  source 1..21: mol_type = other RNA
aaaataagtgtataaaatgca

SEQ ID NO: 814 - RNA, 21 nt, synthetic construct
  source 1..21: mol_type = other RNA
aaataagtgtataaaatgcaa

SEQ ID NO: 815 - RNA, 21 nt, synthetic construct
  source 1..21: mol_type = other RNA
tgtataaaatgcaactgttga

SEQ ID NO: 816 - RNA, 21 nt, synthetic construct
  source 1..21: mol_type = other RNA
caaatcttttctgaagatgaa

SEQ ID NO: 817 - RNA, 21 nt, synthetic construct
  source 1..21: mol_type = other RNA
aatcttttctgaagatgaaga

SEQ ID NO: 818 - RNA, 21 nt, synthetic construct
  source 1..21: mol_type = other RNA
tttctgaagatgaagagttta

SEQ ID NO: 819 - RNA, 21 nt, synthetic construct
  source 1..21: mol_type = other RNA
aagatgaagagtttagtttta

SEQ ID NO: 820 - RNA, 21 nt, synthetic construct
  source 1..21: mol_type = other RNA
agatgaagagtttagttttaa

SEQ ID NO: 821 - RNA, 21 nt, synthetic construct
  source 1..21: mol_type = other RNA
gatgaagagtttagttttaaa

SEQ ID NO: 822 - RNA, 21 nt, synthetic construct
  source 1..21: mol_type = other RNA
atgaagagtttagttttaaaa

SEQ ID NO: 823 - RNA, 21 nt, synthetic construct
  source 1..21: mol_type = other RNA
gagtttagttttaaaactgca

SEQ ID NO: 824 - RNA, 21 nt, synthetic construct
  source 1..21: mol_type = other RNA
agttttaaaactgcactgcca

SEQ ID NO: 825 - RNA, 21 nt, synthetic construct
  source 1..21: mol_type = other RNA
gttttaaaactgcactgccaa

SEQ ID NO: 826 - RNA, 21 nt, synthetic construct
  source 1..21: mol_type = other RNA
tcataagaaagagaaaatata

SEQ ID NO: 827 - RNA, 21 nt, synthetic construct
  source 1..21: mol_type = other RNA
aagaaagagaaaatatattta

SEQ ID NO: 828 - RNA, 21 nt, synthetic construct
  source 1..21: mol_type = other RNA
aaagagaaaatatatttacta

SEQ ID NO: 829 - RNA, 21 nt, synthetic construct
  source 1..21: mol_type = other RNA
agaaaatatatttactattca

SEQ ID NO: 830 - RNA, 21 nt, synthetic construct
  source 1..21: mol_type = other RNA
aaatatatttactattcatta

SEQ ID NO: 831 - RNA, 21 nt, synthetic construct
  source 1..21: mol_type = other RNA
aatatatttactattcattaa

SEQ ID NO: 832 - RNA, 21 nt, synthetic construct
  source 1..21: mol_type = other RNA
atatatttactattcattaaa

SEQ ID NO: 833 - RNA, 21 nt, synthetic construct
  source 1..21: mol_type = other RNA
gcacgtcaaaagctacagaaa

SEQ ID NO: 834 - RNA, 21 nt, synthetic construct
  source 1..21: mol_type = other RNA
cacgtcaaaagctacagaata

SEQ ID NO: 835 - RNA, 21 nt, synthetic construct
  source 1..21: mol_type = other RNA
acgtcaaaagctacagaatca

SEQ ID NO: 836 - RNA, 21 nt, synthetic construct
  source 1..21: mol_type = other RNA
gtcaaaagctacagaatctaa

SEQ ID NO: 837 - RNA, 21 nt, synthetic construct
  source 1..21: mol_type = other RNA
tcaaaagctacagaatctata

SEQ ID NO: 838 - RNA, 21 nt, synthetic construct
  source 1..21: mol_type = other RNA
caaaagctacagaatctatta

SEQ ID NO: 839 - RNA, 21 nt, synthetic construct
  source 1..21: mol_type = other RNA
aaagctacagaatctatttaa

SEQ ID NO: 840 - RNA, 21 nt, synthetic construct
  source 1..21: mol_type = other RNA
aagctacagaatctatttata

SEQ ID NO: 841 - RNA, 21 nt, synthetic construct
  source 1..21: mol_type = other RNA
ctacagaatctatttatcaaa

SEQ ID NO: 842 - RNA, 21 nt, synthetic construct
  source 1..21: mol_type = other RNA
tacagaatctatttatcaata

SEQ ID NO: 843 - RNA, 21 nt, synthetic construct
  source 1..21: mol_type = other RNA
acagaatctatttatcaatta

SEQ ID NO: 844 - RNA, 21 nt, synthetic construct
  source 1..21: mol_type = other RNA
cagaatctatttatcaattta

SEQ ID NO: 845 - RNA, 21 nt, synthetic construct
  source 1..21: mol_type = other RNA
agaatctatttatcaatttca

SEQ ID NO: 846 - RNA, 21 nt, synthetic construct
  source 1..21: mol_type = other RNA
gaatctatttatcaatttcta

SEQ ID NO: 847 - RNA, 21 nt, synthetic construct
  source 1..21: mol_type = other RNA
aatctatttatcaatttctga

SEQ ID NO: 848 - RNA, 21 nt, synthetic construct
  source 1..21: mol_type = other RNA
atctatttatcaatttctgta

SEQ ID NO: 849 - RNA, 21 nt, synthetic construct
  source 1..21: mol_type = other RNA
tctatttatcaatttctgtca

SEQ ID NO: 850 - RNA, 21 nt, synthetic construct
  source 1..21: mol_type = other RNA
ctatttatcaatttctgtcta

SEQ ID NO: 851 - RNA, 21 nt, synthetic construct
  source 1..21: mol_type = other RNA
atttatcaatttctgtctcaa

SEQ ID NO: 852 - RNA, 21 nt, synthetic construct
  source 1..21: mol_type = other RNA
tttatcaatttctgtctcata

SEQ ID NO: 853 - RNA, 21 nt, synthetic construct
  source 1..21: mol_type = other RNA
ttatcaatttctgtctcatca

SEQ ID NO: 854 - RNA, 21 nt, synthetic construct
  source 1..21: mol_type = other RNA
tatcaatttctgtctcatcta

SEQ ID NO: 855 - RNA, 21 nt, synthetic construct
  source 1..21: mol_type = other RNA
caatttctgtctcatcttaaa

SEQ ID NO: 856 - RNA, 21 nt, synthetic construct
  source 1..21: mol_type = other RNA
atttctgtctcatcttaataa

SEQ ID NO: 857 - RNA, 21 nt, synthetic construct
  source 1..21: mol_type = other RNA
tttctgtctcatcttaatata

SEQ ID NO: 858 - RNA, 21 nt, synthetic construct
  source 1..21: mol_type = other RNA
ttctgtctcatcttaatatga

SEQ ID NO: 859 - RNA, 21 nt, synthetic construct
  source 1..21: mol_type = other RNA
tctgtctcatcttaatatgta

SEQ ID NO: 860 - RNA, 21 nt, synthetic construct
  source 1..21: mol_type = other RNA
ctgtctcatcttaatatgtca

SEQ ID NO: 861 - RNA, 21 nt, synthetic construct
  source 1..21: mol_type = other RNA
tgtctcatcttaatatgtcta

SEQ ID NO: 862 - RNA, 21 nt, synthetic construct
  source 1..21: mol_type = other RNA
gtctcatcttaatatgtctca

SEQ ID NO: 863 - RNA, 21 nt, synthetic construct
  source 1..21: mol_type = other RNA
tctcatcttaatatgtctcta

SEQ ID NO: 864 - RNA, 21 nt, synthetic construct
  source 1..21: mol_type = other RNA
ctcatcttaatatgtctctta

SEQ ID NO: 865 - RNA, 21 nt, synthetic construct
  source 1..21: mol_type = other RNA
gaaaaggtcaagattaagaca

SEQ ID NO: 866 - RNA, 21 nt, synthetic construct
  source 1..21: mol_type = other RNA
ggtcaagattaagactaaaaa

SEQ ID NO: 867 - RNA, 21 nt, synthetic construct
  source 1..21: mol_type = other RNA
gtcaagattaagactaaaaca

SEQ ID NO: 868 - RNA, 21 nt, synthetic construct
  source 1..21: mol_type = other RNA
tcaagattaagactaaaacta

SEQ ID NO: 869 - RNA, 21 nt, synthetic construct
  source 1..21: mol_type = other RNA
aagattaagactaaaacttaa

SEQ ID NO: 870 - RNA, 21 nt, synthetic construct
  source 1..21: mol_type = other RNA
agattaagactaaaacttata

SEQ ID NO: 871 - RNA, 21 nt, synthetic construct
  source 1..21: mol_type = other RNA
gattaagactaaaacttatta

SEQ ID NO: 872 - RNA, 21 nt, synthetic construct
  source 1..21: mol_type = other RNA
attaagactaaaacttattga

SEQ ID NO: 873 - RNA, 21 nt, synthetic construct
  source 1..21: mol_type = other RNA
ttaagactaaaacttattgta

SEQ ID NO: 874 - RNA, 21 nt, synthetic construct
  source 1..21: mol_type = other RNA
aagactaaaacttattgttaa

SEQ ID NO: 875 - RNA, 21 nt, synthetic construct
  source 1..21: mol_type = other RNA
agactaaaacttattgttaca

SEQ ID NO: 876 - RNA, 21 nt, synthetic construct
  source 1..21: mol_type = other RNA
actaaaacttattgttaccaa

SEQ ID NO: 877 - RNA, 21 nt, synthetic construct
  source 1..21: mol_type = other RNA
tgttaccatatgtattcatca

SEQ ID NO: 878 - RNA, 21 nt, synthetic construct
  source 1..21: mol_type = other RNA
gttaccatatgtattcatcta

SEQ ID NO: 879 - RNA, 21 nt, synthetic construct
  source 1..21: mol_type = other RNA
ttaccatatgtattcatctga

SEQ ID NO: 880 - RNA, 21 nt, synthetic construct
  source 1..21: mol_type = other RNA
taccatatgtattcatctgta

SEQ ID NO: 881 - RNA, 21 nt, synthetic construct
  source 1..21: mol_type = other RNA
accatatgtattcatctgtta

SEQ ID NO: 882 - RNA, 21 nt, synthetic construct
  source 1..21: mol_type = other RNA
ccatatgtattcatctgttga

SEQ ID NO: 883 - RNA, 21 nt, synthetic construct
  source 1..21: mol_type = other RNA
atatgtattcatctgttggaa

SEQ ID NO: 884 - RNA, 21 nt, synthetic construct
  source 1..21: mol_type = other RNA
tatgtattcatctgttggata

SEQ ID NO: 885 - RNA, 21 nt, synthetic construct
  source 1..21: mol_type = other RNA
atgtattcatctgttggatca

SEQ ID NO: 886 - RNA, 21 nt, synthetic construct
  source 1..21: mol_type = other RNA
tgtattcatctgttggatcta

SEQ ID NO: 887 - RNA, 21 nt, synthetic construct
  source 1..21: mol_type = other RNA
gtattcatctgttggatctta

SEQ ID NO: 888 - RNA, 21 nt, synthetic construct
  source 1..21: mol_type = other RNA
tattcatctgttggatcttga

SEQ ID NO: 889 - RNA, 21 nt, synthetic construct
  source 1..21: mol_type = other RNA
catctgttggatcttgtaaaa

SEQ ID NO: 890 - RNA, 21 nt, synthetic construct
  source 1..21: mol_type = other RNA
tctgttggatcttgtaaacaa

SEQ ID NO: 891 - RNA, 21 nt, synthetic construct
  source 1..21: mol_type = other RNA
ctgttggatcttgtaaacata

SEQ ID NO: 892 - RNA, 21 nt, synthetic construct
  source 1..21: mol_type = other RNA
ggatcttgtaaacatgaaaaa

SEQ ID NO: 893 - RNA, 21 nt, synthetic construct
  source 1..21: mol_type = other RNA
gatcttgtaaacatgaaaaga

SEQ ID NO: 894 - RNA, 21 nt, synthetic construct
  source 1..21: mol_type = other RNA
atcttgtaaacatgaaaagga

SEQ ID NO: 895 - RNA, 21 nt, synthetic construct
  source 1..21: mol_type = other RNA
tcttgtaaacatgaaaaggga

SEQ ID NO: 896 - RNA, 21 nt, synthetic construct
  source 1..21: mol_type = other RNA
cttgtaaacatgaaaagggca

SEQ ID NO: 897 - RNA, 21 nt, synthetic construct
  source 1..21: mol_type = other RNA
aattaacttcaaaataagtga

SEQ ID NO: 898 - RNA, 21 nt, synthetic construct
  source 1..21: mol_type = other RNA
ttaacttcaaaataagtgtaa

SEQ ID NO: 899 - RNA, 21 nt, synthetic construct
  source 1..21: mol_type = other RNA
ttcaaaataagtgtataaaaa

SEQ ID NO: 900 - RNA, 21 nt, synthetic construct
  source 1..21: mol_type = other RNA
tcaaaataagtgtataaaata

SEQ ID NO: 901 - RNA, 21 nt, synthetic construct
  source 1..21: mol_type = other RNA
caaaataagtgtataaaatga

SEQ ID NO: 902 - RNA, 21 nt, synthetic construct
  source 1..21: mol_type = other RNA
aataagtgtataaaatgcaaa

SEQ ID NO: 903 - RNA, 21 nt, synthetic construct
  source 1..21: mol_type = other RNA
ataagtgtataaaatgcaaca

SEQ ID NO: 904 - RNA, 21 nt, synthetic construct
  source 1..21: mol_type = other RNA
taagtgtataaaatgcaacta

SEQ ID NO: 905 - RNA, 21 nt, synthetic construct
  source 1..21: mol_type = other RNA
aagtgtataaaatgcaactga

SEQ ID NO: 906 - RNA, 21 nt, synthetic construct
  source 1..21: mol_type = other RNA
agtgtataaaatgcaactgta

SEQ ID NO: 907 - RNA, 21 nt, synthetic construct
  source 1..21: mol_type = other RNA
gtgtataaaatgcaactgtta

SEQ ID NO: 908 - RNA, 21 nt, synthetic construct
  source 1..21: mol_type = other RNA
aaatcttttctgaagatgaaa

SEQ ID NO: 909 - RNA, 21 nt, synthetic construct
  source 1..21: mol_type = other RNA
atcttttctgaagatgaagaa

SEQ ID NO: 910 - RNA, 21 nt, synthetic construct
  source 1..21: mol_type = other RNA
tcttttctgaagatgaagaga

SEQ ID NO: 911 - RNA, 21 nt, synthetic construct
  source 1..21: mol_type = other RNA
cttttctgaagatgaagagta

SEQ ID NO: 912 - RNA, 21 nt, synthetic construct
  source 1..21: mol_type = other RNA
ttttctgaagatgaagagtta

SEQ ID NO: 913 - RNA, 21 nt, synthetic construct
  source 1..21: mol_type = other RNA
ttctgaagatgaagagtttaa

SEQ ID NO: 914 - RNA, 21 nt, synthetic construct
  source 1..21: mol_type = other RNA
tctgaagatgaagagtttaga

SEQ ID NO: 915 - RNA, 21 nt, synthetic construct
  source 1..21: mol_type = other RNA
ctgaagatgaagagtttagta

SEQ ID NO: 916 - RNA, 21 nt, synthetic construct
  source 1..21: mol_type = other RNA
tgaagatgaagagtttagtta

SEQ ID NO: 917 - RNA, 21 nt, synthetic construct
  source 1..21: mol_type = other RNA
gaagatgaagagtttagttta

SEQ ID NO: 918 - RNA, 21 nt, synthetic construct
  source 1..21: mol_type = other RNA
tgaagagtttagttttaaaaa

SEQ ID NO: 919 - RNA, 21 nt, synthetic construct
  source 1..21: mol_type = other RNA
gaagagtttagttttaaaaca

SEQ ID NO: 920 - RNA, 21 nt, synthetic construct
  source 1..21: mol_type = other RNA
aagagtttagttttaaaacta

SEQ ID NO: 921 - RNA, 21 nt, synthetic construct
  source 1..21: mol_type = other RNA
agagtttagttttaaaactga

SEQ ID NO: 922 - RNA, 21 nt, synthetic construct
  source 1..21: mol_type = other RNA
agtttagttttaaaactgcaa

SEQ ID NO: 923 - RNA, 21 nt, synthetic construct
  source 1..21: mol_type = other RNA
gtttagttttaaaactgcaca

SEQ ID NO: 924 - RNA, 21 nt, synthetic construct
  source 1..21: mol_type = other RNA
tttagttttaaaactgcacta

SEQ ID NO: 925 - RNA, 21 nt, synthetic construct
  source 1..21: mol_type = other RNA
ttagttttaaaactgcactga

SEQ ID NO: 926 - RNA, 21 nt, synthetic construct
  source 1..21: mol_type = other RNA
tagttttaaaactgcactgca

SEQ ID NO: 927 - RNA, 21 nt, synthetic construct
  source 1..21: mol_type = other RNA
atcataagaaagagaaaataa

SEQ ID NO: 928 - RNA, 21 nt, synthetic construct
  source 1..21: mol_type = other RNA
cataagaaagagaaaatataa

SEQ ID NO: 929 - RNA, 21 nt, synthetic construct
  source 1..21: mol_type = other RNA
ataagaaagagaaaatatata

SEQ ID NO: 930 - RNA, 21 nt, synthetic construct
  source 1..21: mol_type = other RNA
taagaaagagaaaatatatta

SEQ ID NO: 931 - RNA, 21 nt, synthetic construct
  source 1..21: mol_type = other RNA
agaaagagaaaatatatttaa

SEQ ID NO: 932 - RNA, 21 nt, synthetic construct
  source 1..21: mol_type = other RNA
gaaagagaaaatatatttaca

SEQ ID NO: 933 - RNA, 21 nt, synthetic construct
  source 1..21: mol_type = other RNA
aagagaaaatatatttactaa

SEQ ID NO: 934 - RNA, 21 nt, synthetic construct
  source 1..21: mol_type = other RNA
agagaaaatatatttactata

SEQ ID NO: 935 - RNA, 21 nt, synthetic construct
  source 1..21: mol_type = other RNA
gagaaaatatatttactatta

SEQ ID NO: 936 - RNA, 21 nt, synthetic construct
  source 1..21: mol_type = other RNA
gaaaatatatttactattcaa

SEQ ID NO: 937 - RNA, 21 nt, synthetic construct
  source 1..21: mol_type = other RNA
aaaatatatttactattcata

SEQ ID NO: 938 - RNA, 21 nt, synthetic construct
  source 1..21: mol_type = other RNA
tatatttactattcattaaaa

SEQ ID NO: 939 - RNA, 23 nt, synthetic construct
  source 1..23: mol_type = other RNA
  modified_base 1: mod_base = OTHER; note = thymine
tttaagatgagacagaaattgat

SEQ ID NO: 940 - RNA, 23 nt, synthetic construct
  source 1..23: mol_type = other RNA
  modified_base 1: mod_base = OTHER; note = thymine
tataaatagattctgtagctttt

SEQ ID NO: 941 - RNA, 23 nt, synthetic construct
  source 1..23: mol_type = other RNA
  modified_base 1: mod_base = OTHER; note = thymine
tttctttgaaaaaattacttaaa

SEQ ID NO: 942 - RNA, 23 nt, synthetic construct
  source 1..23: mol_type = other RNA
  modified_base 1: mod_base = OTHER; note = thymine
ttaagttttagtcttaatcttga

SEQ ID NO: 943 - RNA, 23 nt, synthetic construct
  source 1..23: mol_type = other RNA
  modified_base 1: mod_base = OTHER; note = thymine
tatgtttacaagatccaacagat

SEQ ID NO: 944 - RNA, 23 nt, synthetic construct
  source 1..23: mol_type = other RNA
  modified_base 1: mod_base = OTHER; note = thymine
tcattttatacacttattttgaa

SEQ ID NO: 945 - RNA, 23 nt, synthetic construct
  source 1..23: mol_type = other RNA
  modified_base 1: mod_base = OTHER; note = thymine
tagttttaaaactaaactcttca

SEQ ID NO: 946 - RNA, 23 nt, synthetic construct
  source 1..23: mol_type = other RNA
  modified_base 1: mod_base = OTHER; note = thymine
tctaaactcttcatcttcagaaa

SEQ ID NO: 947 - RNA, 23 nt, synthetic construct
  source 1..23: mol_type = other RNA
  modified_base 1: mod_base = OTHER; note = thymine
ttagtaaatatattttctctttc

SEQ ID NO: 948 - RNA, 23 nt, synthetic construct
  source 1..23: mol_type = other RNA
  modified_base 1: mod_base = OTHER; note = thymine
  modified_base 23: mod_base = OTHER; note = thymine
tttaagatgagacagaaattgat

SEQ ID NO: 949 - RNA, 23 nt, synthetic construct
  source 1..23: mol_type = other RNA
  modified_base 1: mod_base = OTHER; note = thymine
  modified_base 23: mod_base = OTHER; note = thymine
tttaagatgagacagaaattgat

SEQ ID NO: 950 - RNA, 23 nt, synthetic construct
  source 1..23: mol_type = other RNA
  modified_base 1: mod_base = OTHER; note = thymine
  modified_base 23: mod_base = OTHER; note = thymine
tttaagatgagacagaaattgat

SEQ ID NO: 951 - RNA, 23 nt, synthetic construct
  source 1..23: mol_type = other RNA
  modified_base 1: mod_base = OTHER; note = thymine
  modified_base 23: mod_base = OTHER; note = thymine
tttaagatgagacagaaattgat

SEQ ID NO: 952 - RNA, 23 nt, synthetic construct
  source 1..23: mol_type = other RNA
  modified_base 1: mod_base = OTHER; note = thymine
  modified_base 23: mod_base = OTHER; note = thymine
tttaagatgagacagaaattgat

SEQ ID NO: 953 - RNA, 23 nt, synthetic construct
  source 1..23: mol_type = other RNA
  modified_base 1: mod_base = OTHER; note = thymine
tttaagatgagacagaaattgat

SEQ ID NO: 954 - RNA, 23 nt, synthetic construct
  source 1..23: mol_type = other RNA
  modified_base 1: mod_base = OTHER; note = thymine
tttaagatgagacagaaattgat

SEQ ID NO: 955 - RNA, 23 nt, synthetic construct
  source 1..23: mol_type = other RNA
  modified_base 1: mod_base = OTHER; note = thymine
tttaagatgagacagaaattgat

SEQ ID NO: 956 - RNA, 23 nt, synthetic construct
  source 1..23: mol_type = other RNA
  modified_base 1: mod_base = OTHER; note = thymine
tttaagatgagacagaaattgat

SEQ ID NO: 957 - RNA, 23 nt, synthetic construct
  source 1..23: mol_type = other RNA
  modified_base 1: mod_base = OTHER; note = thymine
  modified_base 23: mod_base = OTHER; note = thymine
tttaagatgagacagaaattgat

SEQ ID NO: 958 - RNA, 23 nt, synthetic construct
  source 1..23: mol_type = other RNA
  modified_base 1: mod_base = OTHER; note = thymine
  modified_base 23: mod_base = OTHER; note = thymine
tttaagatgagacagaaattgat

SEQ ID NO: 959 - RNA, 23 nt, synthetic construct
  source 1..23: mol_type = other RNA
  modified_base 1: mod_base = OTHER; note = thymine
tttaagatgagacagaaattgat

SEQ ID NO: 960 - RNA, 23 nt, synthetic construct
  source 1..23: mol_type = other RNA
  modified_base 1: mod_base = OTHER; note = thymine
tttaagatgagacagaaattgat

SEQ ID NO: 961 - RNA, 23 nt, synthetic construct
  source 1..23: mol_type = other RNA
  modified_base 1: mod_base = OTHER; note = thymine
tttaagatgagacagaaattgat

SEQ ID NO: 962 - RNA, 23 nt, synthetic construct
  source 1..23: mol_type = other RNA
  modified_base 1: mod_base = OTHER; note = thymine
tttaagatgagacagaaattgat

SEQ ID NO: 963 - RNA, 23 nt, synthetic construct
  source 1..23: mol_type = other RNA
  modified_base 1: mod_base = OTHER; note = thymine
  modified_base 23: mod_base = OTHER; note = thymine
tttaagatgagacagaaattgat

SEQ ID NO: 964 - RNA, 23 nt, synthetic construct
  source 1..23: mol_type = other RNA
  modified_base 1: mod_base = OTHER; note = thymine
tttaagatgagacagaaattgat

SEQ ID NO: 965 - RNA, 23 nt, synthetic construct
  source 1..23: mol_type = other RNA
  modified_base 1: mod_base = OTHER; note = thymine
  modified_base 23: mod_base = OTHER; note = thymine
tttaagatgagacagaaattgat

SEQ ID NO: 966 - RNA, 23 nt, synthetic construct
  source 1..23: mol_type = other RNA
  modified_base 1: mod_base = OTHER; note = thymine
tttaagatgagacagaaattgaa

SEQ ID NO: 967 - RNA, 23 nt, synthetic construct
  source 1..23: mol_type = other RNA
  modified_base 1: mod_base = OTHER; note = thymine
tttaagatgagacagaaattgaa

SEQ ID NO: 968 - RNA, 23 nt, synthetic construct
  source 1..23: mol_type = other RNA
  modified_base 1: mod_base = OTHER; note = thymine
tttaagatgagacagaaattgaa

SEQ ID NO: 969 - RNA, 23 nt, synthetic construct
  source 1..23: mol_type = other RNA
  modified_base 1: mod_base = OTHER; note = thymine
tttaagatgagacagaaattgaa

SEQ ID NO: 970 - RNA, 23 nt, synthetic construct
  source 1..23: mol_type = other RNA
  modified_base 1: mod_base = OTHER; note = thymine
tttaagatgagacagaaattgaa

SEQ ID NO: 971 - RNA, 23 nt, synthetic construct
  source 1..23: mol_type = other RNA
  modified_base 1: mod_base = OTHER; note = thymine
  modified_base 6: mod_base = OTHER; note = thymine
ttaagttttagtcttaatcttaa

SEQ ID NO: 972 - RNA, 23 nt, synthetic construct
  source 1..23: mol_type = other RNA
  modified_base 1: mod_base = OTHER; note = thymine
  modified_base 7: mod_base = OTHER; note = thymine
ttaagttttagtcttaatcttaa

SEQ ID NO: 973 - RNA, 23 nt, synthetic construct
  source 1..23: mol_type = other RNA
  modified_base 1: mod_base = OTHER; note = thymine
tttaagatgagacagaaattgaa

SEQ ID NO: 974 - RNA, 23 nt, synthetic construct
  source 1..23: mol_type = other RNA
  modified_base 1: mod_base = OTHER; note = thymine
tataaatagattctgtagcttaa

SEQ ID NO: 975 - RNA, 23 nt, synthetic construct
  source 1..23: mol_type = other RNA
  modified_base 1: mod_base = OTHER; note = thymine
  modified_base 7: mod_base = OTHER; note = thymine
tataaatagattctgtagcttaa

SEQ ID NO: 976 - RNA, 21 nt, synthetic construct
  source 1..21: mol_type = other RNA
  modified_base 1: mod_base = OTHER; note = thymine
tttaagatgagacagaaataa

SEQ ID NO: 977 - RNA, 21 nt, synthetic construct
  source 1..21: mol_type = other RNA
aagctacagaatctatttata

SEQ ID NO: 978 - RNA, 21 nt, synthetic construct
  source 1..21: mol_type = other RNA
caatttctgtctcatcttaaa

SEQ ID NO: 979 - RNA, 21 nt, synthetic construct
  source 1..21: mol_type = other RNA
taagtaattttttcaaagaaa

SEQ ID NO: 980 - RNA, 21 nt, synthetic construct
  source 1..21: mol_type = other RNA
aagattaagactaaaacttaa

SEQ ID NO: 981 - RNA, 21 nt, synthetic construct
  source 1..21: mol_type = other RNA
ctgttggatcttgtaaacata

SEQ ID NO: 982 - RNA, 21 nt, synthetic construct
  source 1..21: mol_type = other RNA
caaaataagtgtataaaatga

SEQ ID NO: 983 - RNA, 21 nt, synthetic construct
  source 1..21: mol_type = other RNA
aagagtttagttttaaaacta

SEQ ID NO: 984 - RNA, 21 nt, synthetic construct
  source 1..21: mol_type = other RNA
tctgaagatgaagagtttaga

SEQ ID NO: 985 - RNA, 21 nt, synthetic construct
  source 1..21: mol_type = other RNA
aagagaaaatatatttactaa

SEQ ID NO: 986 - RNA, 21 nt, synthetic construct
  source 1..21: mol_type = other RNA
caatttctgtctcatcttaaa

SEQ ID NO: 987 - RNA, 21 nt, synthetic construct
  source 1..21: mol_type = other RNA
caatttctgtctcatcttaaa

SEQ ID NO: 988 - RNA, 21 nt, synthetic construct
  source 1..21: mol_type = other RNA
caatttctgtctcatcttaaa

SEQ ID NO: 989 - RNA, 21 nt, synthetic construct
  source 1..21: mol_type = other RNA
caatttctgtctcatcttaaa

SEQ ID NO: 990 - RNA, 21 nt, synthetic construct
  source 1..21: mol_type = other RNA
caatttctgtctcatcttaaa

SEQ ID NO: 991 - RNA, 21 nt, synthetic construct
  source 1..21: mol_type = other RNA
caatttctgtctcatcttaaa

SEQ ID NO: 992 - RNA, 21 nt, synthetic construct
  source 1..21: mol_type = other RNA
caatttctgtctcatcttaaa

SEQ ID NO: 993 - RNA, 21 nt, synthetic construct
  source 1..21: mol_type = other RNA
caatttctgtctcatcttaaa

SEQ ID NO: 994 - RNA, 21 nt, synthetic construct
  source 1..21: mol_type = other RNA
caatttctgtctcatcttaaa

SEQ ID NO: 995 - RNA, 21 nt, synthetic construct
  source 1..21: mol_type = other RNA
caatttctgtctcatcttaaa

SEQ ID NO: 996 - RNA, 21 nt, synthetic construct
  source 1..21: mol_type = other RNA
caatttctgtctcatcttaaa

SEQ ID NO: 997 - RNA, 21 nt, synthetic construct
  source 1..21: mol_type = other RNA
caatttctgtctcatcttaaa

SEQ ID NO: 998 - RNA, 19 nt, synthetic construct
  source 1..19: mol_type = other RNA
atttctgtctcatcttaaa

SEQ ID NO: 999 - RNA, 19 nt, synthetic construct
  source 1..19: mol_type = other RNA
atttctgtctcatcttaaa

SEQ ID NO: 1000 - RNA, 19 nt, synthetic construct
  source 1..19: mol_type = other RNA
atttctgtctcatcttaaa

SEQ ID NO: 1001 - RNA, 21 nt, synthetic construct
  source 1..21: mol_type = other RNA
caatttctgtctcatcttaaa

SEQ ID NO: 1002 - RNA, 21 nt, synthetic construct
  source 1..21: mol_type = other RNA
aagctacagaatctatttata

SEQ ID NO: 1003 - RNA, 21 nt, synthetic construct
  source 1..21: mol_type = other RNA
taagtaattttttcaaagaaa

SEQ ID NO: 1004 - RNA, 21 nt, synthetic construct
  source 1..21: mol_type = other RNA
aagattaagactaaaacttaa

SEQ ID NO: 1005 - RNA, 21 nt, synthetic construct
  source 1..21: mol_type = other RNA
ctgttggatcttgtaaacata

SEQ ID NO: 1006 - RNA, 21 nt, synthetic construct
  source 1..21: mol_type = other RNA
caaaataagtgtataaaatga

SEQ ID NO: 1007 - RNA, 21 nt, synthetic construct
  source 1..21: mol_type = other RNA
aagagtttagttttaaaacta

SEQ ID NO: 1008 - RNA, 21 nt, synthetic construct
  source 1..21: mol_type = other RNA
tctgaagatgaagagtttaga

SEQ ID NO: 1009 - RNA, 21 nt, synthetic construct
  source 1..21: mol_type = other RNA
aagagaaaatatatttactaa

SEQ ID NO: 1010 - RNA, 21 nt, synthetic construct
  source 1..21: mol_type = other RNA
caatttctgtctcatcttaaa

SEQ ID NO: 1011 - RNA, 21 nt, synthetic construct
  source 1..21: mol_type = other RNA
caatttctgtctcatcttaaa

SEQ ID NO: 1012 - RNA, 21 nt, synthetic construct
  source 1..21: mol_type = other RNA
caatttctgtctcatcttaaa

SEQ ID NO: 1013 - RNA, 21 nt, synthetic construct
  source 1..21: mol_type = other RNA
  modified_base 20: mod_base = OTHER; note = thymine
aagctacagaatctatttata

SEQ ID NO: 1014 - RNA, 21 nt, synthetic construct
  source 1..21: mol_type = other RNA
aagctacagaatctatttata

SEQ ID NO: 1015 - RNA, 21 nt, synthetic construct
  source 1..21: mol_type = other RNA
aagattaagactaaaacttaa

SEQ ID NO: 1016 - RNA, 21 nt, synthetic construct
  source 1..21: mol_type = other RNA
aagattaagactaaaacttaa

SEQ ID NO: 1017 - DNA, 16 bp, synthetic construct
  source 1..16: mol_type = other DNA
acgagtatattaggaa

SEQ ID NO: 1018 - DNA, 16 bp, synthetic construct
  source 1..16: mol_type = other DNA
acgagtatattaggaa

SEQ ID NO: 1019 - DNA, 16 bp, synthetic construct
  source 1..16: mol_type = other DNA
tgagaattgtagaggt

SEQ ID NO: 1020 - DNA, 16 bp, synthetic construct
  source 1..16: mol_type = other DNA
tgagaattgtagaggt

SEQ ID NO: 1021 - DNA, 16 bp, synthetic construct
  source 1..16: mol_type = other DNA
tgagaattgtagaggt

SEQ ID NO: 1022 - DNA, 16 bp, synthetic construct
  source 1..16: mol_type = other DNA
tgagaattgtagaggt

SEQ ID NO: 1023 - DNA, 16 bp, synthetic construct
  source 1..16: mol_type = other DNA
tgagaattgtagaggt

SEQ ID NO: 1024 - DNA, 16 bp, synthetic construct
  source 1..16: mol_type = other DNA
acacgagtatattagg

SEQ ID NO: 1025 - DNA, 16 bp, synthetic construct
  source 1..16: mol_type = other DNA
acacgagtatattagg

SEQ ID NO: 1026 - protein, 13 aa, synthetic construct
CXXDXXXGCISYC

SEQ ID NO: 1027 - RNA, 21 nt, synthetic construct
  source 1..21: mol_type = other RNA
  modified_base 1: mod_base = OTHER; note = 2'-O-methoxyethyl 5-methylcytidine 3'-phosphorothioate
  modified_base 2: mod_base = OTHER; note = 2'-O-methoxyethyl adenosine 3'-phosphorothioate
  modified_base 3: mod_base = OTHER; note = 2'-O-methyl adenosine 3'-phosphodiester
  modified_base 4..6: mod_base = OTHER; note = 2'-O-methyl uridine 3'-phosphodiester
  modified_base 7: mod_base = OTHER; note = 2'-O-methyl cytidine 3'-phosphodiester
  modified_base 8: mod_base = OTHER; note = 2'-O-methyl uridine 3'-phosphodiester
  modified_base 9: mod_base = OTHER; note = 2'-O-methyl guanosine 3'-phosphodiester
  modified_base 10: mod_base = OTHER; note = 2'-fluoro uridine 3'-phosphodiester
  modified_base 11: mod_base = OTHER; note = 2'-fluoro cytidine 3'-phosphodiester
  modified_base 12: mod_base = OTHER; note = 2'-O-methyl uridine 3'-phosphodiester
  modified_base 13: mod_base = OTHER; note = 2'-O-methyl cytidine 3'-phosphodiester
  modified_base 14: mod_base = OTHER; note = 2'-O-methyl adenosine 3'-phosphodiester
  modified_base 15: mod_base = OTHER; note = 2'-O-methyl uridine 3'-phosphodiester
  modified_base 16: mod_base = OTHER; note = 2'-O-methyl cytidine 3'-phosphodiester
  modified_base 17..18: mod_base = OTHER; note = 2'-O-methyl uridine 3'-phosphodiester
  modified_base 19: mod_base = OTHER; note = 2'-O-methyl adenosine 3'-phosphorothioate
  modified_base 20: mod_base = OTHER; note = 2'-O-methoxyethyl adenosine 3'-phosphorothioate
  modified_base 21: mod_base = OTHER; note = 2'-O-methoxyethyl adenosine
caatttctgtctcatcttaaa

SEQ ID NO: 1028 - RNA, 21 nt, synthetic construct
  source 1..21: mol_type = other RNA
  modified_base 1: mod_base = OTHER; note = 2'-O-methoxyethyl cytidine 3'-phosphorothioate
  modified_base 2: mod_base = OTHER; note = 2'-O-methoxyethyl adenosine 3'-phosphorothioate
  modified_base 3: mod_base = OTHER; note = 2'-O-methyl adenosine 3'-phosphodiester
  modified_base 4..6: mod_base = OTHER; note = 2'-O-methyl uridine 3'-phosphodiester
  modified_base 7: mod_base = OTHER; note = 2'-fluoro cytidine 3'-phosphodiester
  modified_base 8: mod_base = OTHER; note = 2'-O-methyl uridine 3'-phosphodiester
  modified_base 9: mod_base = OTHER; note = 2'-fluoro guanosine 3'-phosphodiester
  modified_base 10: mod_base = OTHER; note = 2'-O-methyl uridine 3'-phosphodiester
  modified_base 11: mod_base = OTHER; note = 2'-fluoro cytidine 3'-phosphodiester
  modified_base 12: mod_base = OTHER; note = 2'-O-methyl uridine 3'-phosphodiester
  modified_base 13: mod_base = OTHER; note = 2'-O-methyl cytidine 3'-phosphodiester
  modified_base 14: mod_base = OTHER; note = 2'-O-methyl adenosine 3'-phosphodiester
  modified_base 15: mod_base = OTHER; note = 2'-O-methyl uridine 3'-phosphodiester
  modified_base 16: mod_base = OTHER; note = 2'-O-methyl cytidine 3'-phosphodiester
  modified_base 17..18: mod_base = OTHER; note = 2'-O-methyl uridine 3'-phosphodiester
  modified_base 19: mod_base = OTHER; note = 2'-O-methyl adenosine 3'-phosphorothioate
  modified_base 20: mod_base = OTHER; note = 2'-O-methoxyethyl adenosine 3'-phosphorothioate
  modified_base 21: mod_base = OTHER; note = 2'-O-methoxyethyl adenosine
caatttctgtctcatcttaaa

SEQ ID NO: 1029 - RNA, 21 nt, synthetic construct
  source 1..21: mol_type = other RNA
  modified_base 20: mod_base = OTHER; note = thymine
  modified_base 1..2: mod_base = OTHER; note = 2'-O-methoxyethyl adenosine 3'-phosphorothioate
  modified_base 3: mod_base = OTHER; note = 2'-O-methyl guanosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-O-methyl adenosine 3'-phosphodiester
  modified_base 7: mod_base = OTHER; note = 2'-O-methyl cytidine 3'-phosphodiester
  modified_base 8: mod_base = OTHER; note = 2'-O-methyl adenosine 3'-phosphodiester
  modified_base 9: mod_base = OTHER; note = 2'-O-methyl guanosine 3'-phosphodiester
  modified_base 10..11: mod_base = OTHER; note = 2'-fluoro adenosine 3'-phosphodiester
  modified_base 12: mod_base = OTHER; note = 2'-O-methyl uridine 3'-phosphodiester
  modified_base 13: mod_base = OTHER; note = 2'-O-methyl cytidine 3'-phosphodiester
  modified_base 14: mod_base = OTHER; note = 2'-O-methyl uridine 3'-phosphodiester
  modified_base 15: mod_base = OTHER; note = 2'-O-methyl adenosine 3'-phosphodiester
  modified_base 16..18: mod_base = OTHER; note = 2'-O-methyl uridine 3'-phosphodiester
  modified_base 19: mod_base = OTHER; note = 2'-O-methyl adenosine 3'-phosphorothioate
  modified_base 20: mod_base = OTHER; note = 2'-O-methoxyethyl thymidine 3'-phosphorothioate
  modified_base 21: mod_base = OTHER; note = 2'-O-methoxyethyl adenosine
aagctacagaatctatttata

SEQ ID NO: 1030 - RNA, 21 nt, synthetic construct
  source 1..21: mol_type = other RNA
  modified_base 1..2: mod_base = OTHER; note = 2'-O-methoxyethyl adenosine 3'-phosphorothioate
  modified_base 3: mod_base = OTHER; note = 2'-O-methyl guanosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-O-methyl adenosine 3'-phosphodiester
  modified_base 7: mod_base = OTHER; note = 2'-fluoro cytidine 3'-phosphodiester
  modified_base 8: mod_base = OTHER; note = 2'-O-methyl adenosine 3'-phosphodiester
  modified_base 9: mod_base = OTHER; note = 2'-fluoro guanosine 3'-phosphodiester
  modified_base 10: mod_base = OTHER; note = 2'-O-methyl adenosine 3'-phosphodiester
  modified_base 11: mod_base = OTHER; note = 2'-fluoro adenosine 3'-phosphodiester
  modified_base 12: mod_base = OTHER; note = 2'-O-methyl uridine 3'-phosphodiester
  modified_base 13: mod_base = OTHER; note = 2'-O-methyl cytidine 3'-phosphodiester
  modified_base 14: mod_base = OTHER; note = 2'-O-methyl uridine 3'-phosphodiester
  modified_base 15: mod_base = OTHER; note = 2'-O-methyl adenosine 3'-phosphodiester
  modified_base 16..18: mod_base = OTHER; note = 2'-O-methyl uridine 3'-phosphodiester
  modified_base 19: mod_base = OTHER; note = 2'-O-methyl adenosine 3'-phosphorothioate
  modified_base 20: mod_base = OTHER; note = 2'-O-methoxyethyl uridine 3'-phosphorothioate
  modified_base 21: mod_base = OTHER; note = 2'-O-methoxyethyl adenosine
aagctacagaatctatttata

SEQ ID NO: 1031 - RNA, 21 nt, synthetic construct
  source 1..21: mol_type = other RNA
  modified_base 1..2: mod_base = OTHER; note = 2'-O-methoxyethyl adenosine 3'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5..6: mod_base = OTHER; note = 2'-O-methyl uridine 3'-phosphodiester
  modified_base 7..8: mod_base = OTHER; note = 2'-O-methyl adenosine 3'-phosphodiester
  modified_base 9: mod_base = OTHER; note = 2'-O-methyl guanosine 3'-phosphodiester
  modified_base 10: mod_base = OTHER; note = 2'-fluoro adenosine 3'-phosphodiester
  modified_base 11: mod_base = OTHER; note = 2'-fluoro cytidine 3'-phosphodiester
  modified_base 12: mod_base = OTHER; note = 2'-O-methyl uridine 3'-phosphodiester
  modified_base 13..16: mod_base = OTHER; note = 2'-O-methyl adenosine 3'-phosphodiester
  modified_base 17: mod_base = OTHER; note = 2'-O-methyl cytidine 3'-phosphodiester
  modified_base 18: mod_base = OTHER; note = 2'-O-methyl uridine 3'-phosphodiester
  modified_base 19: mod_base = OTHER; note = 2'-O-methyl uridine 3'-phosphorothioate
  modified_base 20: mod_base = OTHER; note = 2'-O-methoxyethyl adenosine 3'-phosphorothioate
  modified_base 21: mod_base = OTHER; note = 2'-O-methoxyethyl adenosine
aagattaagactaaaacttaa

SEQ ID NO: 1032 - RNA, 21 nt, synthetic construct
  source 1..21: mol_type = other RNA
  modified_base 1..2: mod_base = OTHER; note = 2'-O-methoxyethyl adenosine 3'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5..6: mod_base = OTHER; note = 2'-O-methyl uridine 3'-phosphodiester
  modified_base 7: mod_base = OTHER; note = 2'-fluoro adenosine 3'-phosphodiester
  modified_base 8: mod_base = OTHER; note = 2'-O-methyl adenosine 3'-phosphodiester
  modified_base 9: mod_base = OTHER; note = 2'-fluoro guanosine 3'-phosphodiester
  modified_base 10: mod_base = OTHER; note = 2'-O-methyl adenosine 3'-phosphodiester
  modified_base 11: mod_base = OTHER; note = 2'-fluoro cytidine 3'-phosphodiester
  modified_base 12: mod_base = OTHER; note = 2'-O-methyl uridine 3'-phosphodiester
  modified_base 13..16: mod_base = OTHER; note = 2'-O-methyl adenosine 3'-phosphodiester
  modified_base 17: mod_base = OTHER; note = 2'-O-methyl cytidine 3'-phosphodiester
  modified_base 18: mod_base = OTHER; note = 2'-O-methyl uridine 3'-phosphodiester
  modified_base 19: mod_base = OTHER; note = 2'-O-methyl uridine 3'-phosphorothioate
  modified_base 20: mod_base = OTHER; note = 2'-O-methoxyethyl adenosine 3'-phosphorothioate
  modified_base 21: mod_base = OTHER; note = 2'-O-methoxyethyl adenosine
aagattaagactaaaacttaa

SEQ ID NO: 1033 - RNA, 23 nt, synthetic construct
  source 1..23: mol_type = other RNA
  modified_base 1: mod_base = OTHER; note = thymine
  modified_base <1: mod_base = OTHER; note = vinyl phosphonate
  modified_base 1: mod_base = OTHER; note = 2'-O-methoxyethyl thymidine 3'-phosphorothioate
  modified_base 2: mod_base = OTHER; note = 2'-fluoro uridine 3'-phosphorothioate
  modified_base 3: mod_base = OTHER; note = 2'-O-methyl uridine 3'-phosphodiester
  modified_base 4..5: mod_base = OTHER; note = 2'-O-methyl adenosine 3'-phosphodiester
  modified_base 6: mod_base = OTHER; note = 2'-deoxyguanosine 3'-phosphodiester
  modified_base 7: mod_base = OTHER; note = 2'-O-methyl adenosine 3'-phosphodiester
  modified_base 8: mod_base = OTHER; note = 2'-O-methyl uridine 3'-phosphodiester
  modified_base 9: mod_base = OTHER; note = 2'-O-methyl guanosine 3'-phosphodiester
  modified_base 10: mod_base = OTHER; note = 2'-O-methyl adenosine 3'-phosphodiester
  modified_base 11: mod_base = OTHER; note = 2'-O-methyl guanosine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro adenosine 3'-phosphodiester
  modified_base 15: mod_base = OTHER; note = 2'-O-methyl guanosine 3'-phosphodiester
  modified_base 16: mod_base = OTHER; note = 2'-fluoro adenosine 3'-phosphodiester
  modified_base 17..18: mod_base = OTHER; note = 2'-O-methyl adenosine 3'-phosphodiester
  modified_base 19..20: mod_base = OTHER; note = 2'-O-methyl uridine 3'-phosphodiester
  modified_base 21: mod_base = OTHER; note = 2'-O-methyl guanosine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tttaagatgagacagaaattgaa

SEQ ID NO: 1034 - RNA, 23 nt, synthetic construct
  source 1..23: mol_type = other RNA
  modified_base 1: mod_base = OTHER; note = thymine
  modified_base <1: mod_base = OTHER; note = vinyl phosphonate
  modified_base 1: mod_base = OTHER; note = 2'-O-methoxyethyl thymidine 3'-phosphorothioate
  modified_base 2: mod_base = OTHER; note = 2'-fluoro uridine 3'-phosphorothioate
  modified_base 3: mod_base = OTHER; note = 2'-O-methyl uridine 3'-phosphodiester
  modified_base 4: mod_base = OTHER; note = 2'-O-methyl adenosine 3'-phosphodiester
  modified_base 5: mod_base = OTHER; note = 2'-deoxyadenosine 3'-phosphodiester
  modified_base 6: mod_base = OTHER; note = 2'-O-methyl guanosine 3'-phosphodiester
  modified_base 7: mod_base = OTHER; note = 2'-deoxyadenosine 3'-phosphodiester
  modified_base 8: mod_base = OTHER; note = 2'-O-methyl uridine 3'-phosphodiester
  modified_base 9: mod_base = OTHER; note = 2'-O-methyl guanosine 3'-phosphodiester
  modified_base 10: mod_base = OTHER; note = 2'-O-methyl adenosine 3'-phosphodiester
  modified_base 11: mod_base = OTHER; note = 2'-O-methyl guanosine 3'-phosphodiester
  modified_base 12: mod_base = OTHER; note = 2'-O-methyl adenosine 3'-phosphodiester
  modified_base 13: mod_base = OTHER; note = 2'-O-methyl cytidine 3'-phosphodiester
  modified_base 14: mod_base = OTHER; note = 2'-fluoro adenosine 3'-phosphodiester
  modified_base 15: mod_base = OTHER; note = 2'-O-methyl guanosine 3'-phosphodiester
  modified_base 16: mod_base = OTHER; note = 2'-fluoro adenosine 3'-phosphodiester
  modified_base 17..18: mod_base = OTHER; note = 2'-O-methyl adenosine 3'-phosphodiester
  modified_base 19..20: mod_base = OTHER; note = 2'-O-methyl uridine 3'-phosphodiester
  modified_base 21: mod_base = OTHER; note = 2'-O-methyl guanosine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tttaagatgagacagaaattgaa

SEQ ID NO: 1035 - RNA, 23 nt, synthetic construct
  source 1..23: mol_type = other RNA
  modified_base 1: mod_base = OTHER; note = thymine
  modified_base <1: mod_base = OTHER; note = vinyl phosphonate
  modified_base 1: mod_base = OTHER; note = 2'-O-methoxyethyl thymidine 3'-phosphorothioate
  modified_base 2: mod_base = OTHER; note = 2'-fluoro adenosine 3'-phosphorothioate
  modified_base 3: mod_base = OTHER; note = 2'-O-methyl uridine 3'-phosphodiester
  modified_base 4..5: mod_base = OTHER; note = 2'-O-methyl adenosine 3'-phosphodiester
  modified_base 6: mod_base = OTHER; note = 2'-deoxyadenosine 3'-phosphodiester
  modified_base 7: mod_base = OTHER; note = 2'-O-methyl uridine 3'-phosphodiester
  modified_base 8: mod_base = OTHER; note = 2'-O-methyl adenosine 3'-phosphodiester
  modified_base 9: mod_base = OTHER; note = 2'-O-methyl guanosine 3'-phosphodiester
  modified_base 10: mod_base = OTHER; note = 2'-O-methyl adenosine 3'-phosphodiester
  modified_base 11..12: mod_base = OTHER; note = 2'-O-methyl uridine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17: mod_base = OTHER; note = 2'-O-methyl adenosine 3'-phosphodiester
  modified_base 18: mod_base = OTHER; note = 2'-O-methyl guanosine 3'-phosphodiester
  modified_base 19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tataaatagattctgtagcttaa

SEQ ID NO: 1036 - RNA, 23 nt, synthetic construct
  source 1..23: mol_type = other RNA
  modified_base 1: mod_base = OTHER; note = thymine
  modified_base 7: mod_base = OTHER; note = thymine
  modified_base <1: mod_base = OTHER; note = vinyl phosphonate
  modified_base 1: mod_base = OTHER; note = 2'-O-methoxyethyl thymidine 3'-phosphorothioate
  modified_base 2: mod_base = OTHER; note = 2'-fluoro adenosine 3'-phosphorothioate
  modified_base 3: mod_base = OTHER; note = 2'-O-methyl uridine 3'-phosphodiester
  modified_base 4: mod_base = OTHER; note = 2'-O-methyl adenosine 3'-phosphodiester
  modified_base 5: mod_base = OTHER; note = 2'-deoxyadenosine 3'-phosphodiester
  modified_base 6: mod_base = OTHER; note = 2'-O-methyl adenosine 3'-phosphodiester
  modified_base 7: mod_base = OTHER; note = 2'-deoxythymidine 3'-phosphodiester
  modified_base 8: mod_base = OTHER; note = 2'-O-methyl adenosine 3'-phosphodiester
  modified_base 9: mod_base = OTHER; note = 2'-O-methyl guanosine 3'-phosphodiester
  modified_base 10: mod_base = OTHER; note = 2'-O-methyl adenosine 3'-phosphodiester
  modified_base 11..12: mod_base = OTHER; note = 2'-O-methyl uridine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17: mod_base = OTHER; note = 2'-O-methyl adenosine 3'-phosphodiester
  modified_base 18: mod_base = OTHER; note = 2'-O-methyl guanosine 3'-phosphodiester
  modified_base 19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tataaatagattctgtagcttaa

SEQ ID NO: 1037 - RNA, 23 nt, synthetic construct
  source 1..23: mol_type = other RNA
  modified_base 1: mod_base = OTHER; note = thymine
  modified_base 6: mod_base = OTHER; note = thymine
  modified_base <1: mod_base = OTHER; note = vinyl phosphonate
  modified_base 1: mod_base = OTHER; note = 2'-O-methoxyethyl thymidine 3'-phosphorothioate
  modified_base 2: mod_base = OTHER; note = 2'-fluoro uridine 3'-phosphorothioate
  modified_base 3..4: mod_base = OTHER; note = 2'-O-methyl adenosine 3'-phosphodiester
  modified_base 5: mod_base = OTHER; note = 2'-O-methyl guanosine 3'-phosphodiester
  modified_base 6: mod_base = OTHER; note = 2'-deoxythymidine 3'-phosphodiester
  modified_base 7..9: mod_base = OTHER; note = 2'-O-methyl uridine 3'-phosphodiester
  modified_base 10: mod_base = OTHER; note = 2'-O-methyl adenosine 3'-phosphodiester
  modified_base 11: mod_base = OTHER; note = 2'-O-methyl guanosine 3'-phosphodiester
  modified_base 12: mod_base = OTHER; note = 2'-O-methyl uridine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl uridine 3'-phosphodiester
  modified_base 16: mod_base = OTHER; note = 2'-fluoro adenosine 3'-phosphodiester
  modified_base 17: mod_base = OTHER; note = 2'-O-methyl adenosine 3'-phosphodiester
  modified_base 18: mod_base = OTHER; note = 2'-O-methyl uridine 3'-phosphodiester
  modified_base 19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
ttaagttttagtcttaatcttaa

SEQ ID NO: 1038 - RNA, 23 nt, synthetic construct
  source 1..23: mol_type = other RNA
  modified_base 1: mod_base = OTHER; note = thymine
  modified_base 7: mod_base = OTHER; note = thymine
  modified_base <1: mod_base = OTHER; note = vinyl phosphonate
  modified_base 1: mod_base = OTHER; note = 2'-O-methoxyethyl thymidine 3'-phosphorothioate
  modified_base 2: mod_base = OTHER; note = 2'-fluoro uridine 3'-phosphorothioate
  modified_base 3..4: mod_base = OTHER; note = 2'-O-methyl adenosine 3'-phosphodiester
  modified_base 5: mod_base = OTHER; note = 2'-deoxyguanosine 3'-phosphodiester
  modified_base 6: mod_base = OTHER; note = 2'-O-methyl uridine 3'-phosphodiester
  modified_base 7: mod_base = OTHER; note = 2'-deoxythymidine 3'-phosphodiester
  modified_base 8..9: mod_base = OTHER; note = 2'-O-methyl uridine 3'-phosphodiester
  modified_base 10: mod_base = OTHER; note = 2'-O-methyl adenosine 3'-phosphodiester
  modified_base 11: mod_base = OTHER; note = 2'-O-methyl guanosine 3'-phosphodiester
  modified_base 12: mod_base = OTHER; note = 2'-O-methyl uridine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl uridine 3'-phosphodiester
  modified_base 16: mod_base = OTHER; note = 2'-fluoro adenosine 3'-phosphodiester
  modified_base 17: mod_base = OTHER; note = 2'-O-methyl adenosine 3'-phosphodiester
  modified_base 18: mod_base = OTHER; note = 2'-O-methyl uridine 3'-phosphodiester
  modified_base 19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
ttaagttttagtcttaatcttaa

SEQ ID NO: 1039 - RNA, 21 nt, synthetic construct
  source 1..21: mol_type = other RNA
  modified_base <1: mod_base = OTHER; note = [X]n , wherein [X] = a conjugate group comprising a bicycle ligand, and n and k (at >21) are 0 or 1, wherein if n=1 then k=0, and if n=0 then k=1
  modified_base 1: mod_base = OTHER; note = 2'-O-methoxyethyl 5-methylcytidine 3'-phosphorothioate
  modified_base 2: mod_base = OTHER; note = 2'-O-methoxyethyl adenosine 3'-phosphorothioate
  modified_base 3: mod_base = OTHER; note = 2'-O-methyl adenosine 3'-phosphodiester
  modified_base 4..6: mod_base = OTHER; note = 2'-O-methyl uridine 3'-phosphodiester
  modified_base 7: mod_base = OTHER; note = 2'-O-methyl cytidine 3'-phosphodiester
  modified_base 8: mod_base = OTHER; note = 2'-O-methyl uridine 3'-phosphodiester
  modified_base 9: mod_base = OTHER; note = 2'-O-methyl guanosine 3'-phosphodiester
  modified_base 10: mod_base = OTHER; note = 2'-fluoro uridine 3'-phosphodiester
  modified_base 11: mod_base = OTHER; note = 2'-fluoro cytidine 3'-phosphodiester
  modified_base 12: mod_base = OTHER; note = 2'-O-methyl uridine 3'-phosphodiester
  modified_base 13: mod_base = OTHER; note = 2'-O-methyl cytidine 3'-phosphodiester
  modified_base 14: mod_base = OTHER; note = 2'-O-methyl adenosine 3'-phosphodiester
  modified_base 15: mod_base = OTHER; note = 2'-O-methyl uridine 3'-phosphodiester
  modified_base 16: mod_base = OTHER; note = 2'-O-methyl cytidine 3'-phosphodiester
  modified_base 17..18: mod_base = OTHER; note = 2'-O-methyl uridine 3'-phosphodiester
  modified_base 19: mod_base = OTHER; note = 2'-O-methyl adenosine 3'-phosphorothioate
  modified_base 20: mod_base = OTHER; note = 2'-O-methoxyethyl adenosine 3'-phosphorothioate
  modified_base 21: mod_base = OTHER; note = 2'-O-methoxyethyl adenosine
  modified_base >21: mod_base = OTHER; note = [X]k , wherein [X] = a conjugate group comprising a bicycle ligand, and n (at <1) and k are 0 or 1, wherein if; n = 1 then k=0, and if n=0 then k=1
caatttctgtctcatcttaaa

SEQ ID NO: 1040 - RNA, 21 nt, synthetic construct
  source 1..21: mol_type = other RNA
  modified_base <1: mod_base = OTHER; note = [X]n , wherein [X] = a conjugate group comprising a bicycle ligand, and n and k (at >21) are 0 or 1, wherein if n=1 then k=0, and if n=0 then k=1
  modified_base 1: mod_base = OTHER; note = 2'-O-methoxyethyl cytidine 3'-phosphorothioate
  modified_base 2: mod_base = OTHER; note = 2'-O-methoxyethyl adenosine 3'-phosphorothioate
  modified_base 3: mod_base = OTHER; note = 2'-O-methyl adenosine 3'-phosphodiester
  modified_base 4..6: mod_base = OTHER; note = 2'-O-methyl uridine 3'-phosphodiester
  modified_base 7: mod_base = OTHER; note = 2'-fluoro cytidine 3'-phosphodiester
  modified_base 8: mod_base = OTHER; note = 2'-O-methyl uridine 3'-phosphodiester
  modified_base 9: mod_base = OTHER; note = 2'-fluoro guanosine 3'-phosphodiester
  modified_base 10: mod_base = OTHER; note = 2'-O-methyl uridine 3'-phosphodiester
  modified_base 11: mod_base = OTHER; note = 2'-fluoro cytidine 3'-phosphodiester
  modified_base 12: mod_base = OTHER; note = 2'-O-methyl uridine 3'-phosphodiester
  modified_base 13: mod_base = OTHER; note = 2'-O-methyl cytidine 3'-phosphodiester
  modified_base 14: mod_base = OTHER; note = 2'-O-methyl adenosine 3'-phosphodiester
  modified_base 15: mod_base = OTHER; note = 2'-O-methyl uridine 3'-phosphodiester
  modified_base 16: mod_base = OTHER; note = 2'-O-methyl cytidine 3'-phosphodiester
  modified_base 17..18: mod_base = OTHER; note = 2'-O-methyl uridine 3'-phosphodiester
  modified_base 19: mod_base = OTHER; note = 2'-O-methyl adenosine 3'-phosphorothioate
  modified_base 20: mod_base = OTHER; note = 2'-O-methoxyethyl adenosine 3'-phosphorothioate
  modified_base 21: mod_base = OTHER; note = 2'-O-methoxyethyl adenosine
  modified_base >21: mod_base = OTHER; note = [X]k , wherein [X] = a conjugate group comprising a bicycle ligand, and n (at <1) and k are 0 or 1, wherein if; n = 1 then k=0, and if n=0 then k=1
caatttctgtctcatcttaaa

SEQ ID NO: 1041 - RNA, 21 nt, synthetic construct
  source 1..21: mol_type = other RNA
  modified_base 20: mod_base = OTHER; note = thymine
  modified_base <1: mod_base = OTHER; note = [X]n , wherein [X] = a conjugate group comprising a bicycle ligand, and n and k (at >21) are 0 or 1, wherein if n=1 then k=0, and if n=0 then k=1
  modified_base 1..2: mod_base = OTHER; note = 2'-O-methoxyethyl adenosine 3'-phosphorothioate
  modified_base 3: mod_base = OTHER; note = 2'-O-methyl guanosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-O-methyl adenosine 3'-phosphodiester
  modified_base 7: mod_base = OTHER; note = 2'-O-methyl cytidine 3'-phosphodiester
  modified_base 8: mod_base = OTHER; note = 2'-O-methyl adenosine 3'-phosphodiester
  modified_base 9: mod_base = OTHER; note = 2'-O-methyl guanosine 3'-phosphodiester
  modified_base 10..11: mod_base = OTHER; note = 2'-fluoro adenosine 3'-phosphodiester
  modified_base 12: mod_base = OTHER; note = 2'-O-methyl uridine 3'-phosphodiester
  modified_base 13: mod_base = OTHER; note = 2'-O-methyl cytidine 3'-phosphodiester
  modified_base 14: mod_base = OTHER; note = 2'-O-methyl uridine 3'-phosphodiester
  modified_base 15: mod_base = OTHER; note = 2'-O-methyl adenosine 3'-phosphodiester
  modified_base 16..18: mod_base = OTHER; note = 2'-O-methyl uridine 3'-phosphodiester
  modified_base 19: mod_base = OTHER; note = 2'-O-methyl adenosine 3'-phosphorothioate
  modified_base 20: mod_base = OTHER; note = 2'-O-methoxyethyl thymidine 3'-phosphorothioate
  modified_base 21: mod_base = OTHER; note = 2'-O-methoxyethyl adenosine
  modified_base >21: mod_base = OTHER; note = [X]k , wherein [X] = a conjugate group comprising a bicycle ligand, and n (at <1) and k are 0 or 1, wherein if; n = 1 then k=0, and if n=0 then k=1
aagctacagaatctatttata

SEQ ID NO: 1042 - RNA, 21 nt, synthetic construct
  source 1..21: mol_type = other RNA
  modified_base <1: mod_base = OTHER; note = [X]n , wherein [X] = a conjugate group comprising a bicycle ligand, and n and k (at >21) are 0 or 1, wherein if n=1 then k=0, and if n=0 then k=1
  modified_base 1..2: mod_base = OTHER; note = 2'-O-methoxyethyl adenosine 3'-phosphorothioate
  modified_base 3: mod_base = OTHER; note = 2'-O-methyl guanosine 3'-phosphodiester
  modified_base 4: mod_base = OTHER; note = 2'-O-methyl cytidine 3'-phosphodiester
  modified_base 5: mod_base = OTHER; note = 2'-O-methyl uridine 3'-phosphodiester
  modified_base 6: mod_base = OTHER; note = 2'-O-methyl adenosine 3'-phosphodiester
  modified_base 7: mod_base = OTHER; note = 2'-fluoro cytidine 3'-phosphodiester
  modified_base 8: mod_base = OTHER; note = 2'-O-methyl adenosine 3'-phosphodiester
  modified_base 9: mod_base = OTHER; note = 2'-fluoro guanosine 3'-phosphodiester
  modified_base 10: mod_base = OTHER; note = 2'-O-methyl adenosine 3'-phosphodiester
  modified_base 11: mod_base = OTHER; note = 2'-fluoro adenosine 3'-phosphodiester
  modified_base 12: mod_base = OTHER; note = 2'-O-methyl uridine 3'-phosphodiester
  modified_base 13: mod_base = OTHER; note = 2'-O-methyl cytidine 3'-phosphodiester
  modified_base 14: mod_base = OTHER; note = 2'-O-methyl uridine 3'-phosphodiester
  modified_base 15: mod_base = OTHER; note = 2'-O-methyl adenosine 3'-phosphodiester
  modified_base 16..18: mod_base = OTHER; note = 2'-O-methyl uridine 3'-phosphodiester
  modified_base 19: mod_base = OTHER; note = 2'-O-methyl adenosine 3'-phosphorothioate
  modified_base 20: mod_base = OTHER; note = 2'-O-methoxyethyl uridine 3'-phosphorothioate
  modified_base 21: mod_base = OTHER; note = 2'-O-methoxyethyl adenosine
  modified_base >21: mod_base = OTHER; note = [X]k , wherein [X] = a conjugate group comprising a bicycle ligand, and n (at <1) and k are 0 or 1, wherein if; n = 1 then k=0, and if n=0 then k=1
aagctacagaatctatttata

SEQ ID NO: 1043 - RNA, 21 nt, synthetic construct
  source 1..21: mol_type = other RNA
  modified_base <1: mod_base = OTHER; note = [X]n , wherein [X] = a conjugate group comprising a bicycle ligand, and n and k (at >21) are 0 or 1, wherein if n=1 then k=0, and if n=0 then k=1
  modified_base 1..2: mod_base = OTHER; note = 2'-O-methoxyethyl adenosine 3'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5..6: mod_base = OTHER; note = 2'-O-methyl uridine 3'-phosphodiester
  modified_base 7..8: mod_base = OTHER; note = 2'-O-methyl adenosine 3'-phosphodiester
  modified_base 9: mod_base = OTHER; note = 2'-O-methyl guanosine 3'-phosphodiester
  modified_base 10: mod_base = OTHER; note = 2'-fluoro adenosine 3'-phosphodiester
  modified_base 11: mod_base = OTHER; note = 2'-fluoro cytidine 3'-phosphodiester
  modified_base 12: mod_base = OTHER; note = 2'-O-methyl uridine 3'-phosphodiester
  modified_base 13..16: mod_base = OTHER; note = 2'-O-methyl adenosine 3'-phosphodiester
  modified_base 17: mod_base = OTHER; note = 2'-O-methyl cytidine 3'-phosphodiester
  modified_base 18: mod_base = OTHER; note = 2'-O-methyl uridine 3'-phosphodiester
  modified_base 19: mod_base = OTHER; note = 2'-O-methyl uridine 3'-phosphorothioate
  modified_base 20: mod_base = OTHER; note = 2'-O-methoxyethyl adenosine 3'-phosphorothioate
  modified_base 21: mod_base = OTHER; note = 2'-O-methoxyethyl adenosine
  modified_base >21: mod_base = OTHER; note = [X]k , wherein [X] = a conjugate group comprising a bicycle ligand, and n (at <1) and k are 0 or 1, wherein if; n = 1 then k=0, and if n=0 then k=1
aagattaagactaaaacttaa

SEQ ID NO: 1044 - RNA, 21 nt, synthetic construct
  source 1..21: mol_type = other RNA
  modified_base <1: mod_base = OTHER; note = [X]n , wherein [X] = a conjugate group comprising a bicycle ligand, and n and k (at >21) are 0 or 1, wherein if n=1 then k=0, and if n=0 then k=1
  modified_base 1..2: mod_base = OTHER; note = 2'-O-methoxyethyl adenosine 3'-phosphorothioate
  modified_base 3: mod_base = OTHER; note = 2'-O-methyl guanosine 3'-phosphodiester
  modified_base 4: mod_base = OTHER; note = 2'-O-methyl adenosine 3'-phosphodiester
  modified_base 5..6: mod_base = OTHER; note = 2'-O-methyl uridine 3'-phosphodiester
  modified_base 7: mod_base = OTHER; note = 2'-fluoro adenosine 3'-phosphodiester
  modified_base 8: mod_base = OTHER; note = 2'-O-methyl adenosine 3'-phosphodiester
  modified_base 9: mod_base = OTHER; note = 2'-fluoro guanosine 3'-phosphodiester
  modified_base 10: mod_base = OTHER; note = 2'-O-methyl adenosine 3'-phosphodiester
  modified_base 11: mod_base = OTHER; note = 2'-fluoro cytidine 3'-phosphodiester
  modified_base 12: mod_base = OTHER; note = 2'-O-methyl uridine 3'-phosphodiester
  modified_base 13..16: mod_base = OTHER; note = 2'-O-methyl adenosine 3'-phosphodiester
  modified_base 17: mod_base = OTHER; note = 2'-O-methyl cytidine 3'-phosphodiester
  modified_base 18: mod_base = OTHER; note = 2'-O-methyl uridine 3'-phosphodiester
  modified_base 19: mod_base = OTHER; note = 2'-O-methyl uridine 3'-phosphorothioate
  modified_base 20: mod_base = OTHER; note = 2'-O-methoxyethyl adenosine 3'-phosphorothioate
  modified_base 21: mod_base = OTHER; note = 2'-O-methoxyethyl adenosine
  modified_base >21: mod_base = OTHER; note = [X]k , wherein [X] = a conjugate group comprising a bicycle ligand, and n (at <1) and k are 0 or 1, wherein if; n = 1 then k=0, and if n=0 then k=1
aagattaagactaaaacttaa

SEQ ID NO: 1045 - protein, 17 aa, synthetic construct
  SITE 1: note = Acetylation at N-terminus. Cysteines 1, 9 and 13 are linked by thioether bonds with the molecular scaffold 1,1',1''-(1,3,5-triazinane-1,3,5-triyl)tris(2-bromoethanon e) (TATB)
  SITE 3: note = 4Hyp
  SITE 9: note = Cysteines 1, 9 and 13 are linked by thioether bonds with the molecular scaffold 1,1',1''-(1,3,5-triazinane-1,3 ,5-triyl)tris(2-bromoethanone) (TATB)
  SITE 13: note = Cysteines 1, 9 and 13 are linked by thioether bonds with the molecular scaffold 1,1',1''-(1,3,5-triazinane-1,3 ,5-triyl)tris(2-bromoethanone) (TATB)
  SITE 17: note = 6-azido lysine. Amidation at C-terminus
  SITE 10: note = t-butyl glycine
CPXDAYLGCXSYCEPWX

SEQ ID NO: 1046 - DNA, 22 bp, synthetic construct
  source 1..22: mol_type = other DNA
gaagaacagaagggcagagatt

SEQ ID NO: 1047 - DNA, 22 bp, synthetic construct
  source 1..22: mol_type = other DNA
aaaccagtgaggtgagtgtatc

SEQ ID NO: 1048 - DNA, 26 bp, synthetic construct
  source 1..26: mol_type = other DNA
tttccagcttgtagatgaggcgtcac

SEQ ID NO: 1049 - DNA, 19 bp, synthetic construct
  source 1..19: mol_type = other DNA
gctgccaaggctacctaaa

SEQ ID NO: 1050 - DNA, 23 bp, synthetic construct
  source 1..23: mol_type = other DNA
gagtgaggtattggactttctcc

SEQ ID NO: 1051 - DNA, 26 bp, synthetic construct
  source 1..26: mol_type = other DNA
tcagacttcctgtcctgctggtatca

SEQ ID NO: 1052 - DNA, 14000 bp, Mus musculus
  source 1..14000: mol_type = genomic DNA
ctctttccctttgctgttggatggacccacggatggagaatccacagattgacactaaaccctataacattatctgccaaattattattagcaagagccaaatgcactgctagcattttagtagcttatagtctacagtaaaaaaatgaatgaacagtgctttcctcacttcagctcctcccactgttgggagtttttctcactttcccttaataaatctgcctgctctgcagaaaacaaaactgaataaacaaacatttcactgagagcttagtgaaaatcacaagtaactgtttccacagcatgttcaagggcctgagctatggctacccacggccatgtcaccagttcactttctgaaggcaaaggacactgagcactaacatgaaacagataacttgccacagtctgggaccatcactggatacacttcttgctgctaaccctcagctgcaataccattccacacatctctttgtgtcatgtgcccaatgtggactccagtgataagggattccaaatgaagactgacaagtagttgtacaagttttccaagactcaagtggagcaggttgttagcaacactcaataactgaccagtgtttctactaaacaatctgtgtttacggaatcttggtaaaaacaaacctacctcctaaaactccatcagagtcatcatgaccagcacagcaaaagtaaaggatagtatttctcatacacatctacagaatgtgacttagattccagttttcaactgtcaggactctatagtcccttccttgggtgtgtgtttgtgaaatatgtatttttaaaatcaccttgcaaacatgtaaaataagtatgatatgttggtctgcagcaattccttatcttctccaaccaagaattatcagttctattgttcctaagctttattctcaaataaatacatatatttgacagacaaagcagactttggaaaaatagttacagccctgaataattaccttcagaactgatgtgttaacatatgcagttgtatcagagcaaaatccaactgttggctttgccctccaatatgccaatcattctggaagttataatgtaaacttggctccatcccagacctcctctgcccttcccacctgccacctttgataagctactataagccactgcactttctaaggtttcatattactagcagttatttgcagctgataatgtgaccttttttttttttaatcatataaagacagaaagaaaactgtcttaaggtacagcggaaagtaccaaacaatgacgcttttgcctctacacacagtgacagggaattcttgcaataagatagtctgcactttacttttacgtggtaaatatgtccttaaataatatttatagactttctttgataaaatattttccagttagttttagctaacattttaagcaacaggagatttgctctagagaagtgtttcatcttgctttgcaagaaggcaagatcatatccatttaaggacttcagtgagcagctgcaggcacacagttttcatactcacaggaaagagggaggctgattttactctactatacactaagaattacgcataggttctctaaagctaaacagtgaaactgaaaaatcagggtccacacatgtgctgatttacatacatgtaatataaaaacagtgaagcacggtaggagacagcaggacgtgctcggctctgcagcatggctcagttctactcaaagtcagtccagacctactgtggtggcattctaaaatttttctcacatttcagacaaaaaaaaatgctacattaggtgaaaaactttcagaaataaatactggacacttatttcatctcttggagacctggtcagcaccgccacactgccatacagctgacagaaacctgcagctcatcttttctagaaaagctactcctgacactaggtcagcttactaaaaacctatttggtttctgtggtaatacaatgtagctatagaaaactcttaaggccttaaacgtataaagtgcatctttctggggaataatacatagctattgaaatacagattcatttctcagagcacgggtgaaaaccttaactgaagaaaatgtaagttgaatgtatttgctcctctaactataaatttctttttcaaaacacaaagtattgacctagttttcttttatgcattcattctcttacctagacagcaatacctagacagatgtgaaacacaaacataaataagacaaggaaaggcatcaaatatcacctgagaatgtttgctgctctgacaagcaaggctctgcgagggcagctccattaaccctgcctctgttgagcctacattatccctcccaagctacctgcattccttcacaactatgttagctcttccactccaacagcagtgtgtaactatctccaagtctgacgacttataactctttaaggaaagctacctactataaagcagtatcttcaaacttactttattctcagttatctgttatacacatttttaagttaaaaactaaaagcaaagcacaaagtgttaatggcatttccaaacctaacataaaataagttctaaggtttggttgtgataagaacatggctaaccaatcacatgttagaattcctacgtgatgtcataagacctccctagaatcctttttctcctccacctactgcagctgctcccataaacctgggaacagaaaactgcccagctaagctcccataagacttcatacagcttcatgctctgcactgtgacgatcaccgaagccaaggtaagacgtgagatcattgactttggacattcttactttctgatattcaatcttattaaactcttaaaagaattctaactcccaaattccaattcaaagctacataataaaagaacctgctatgacaaaatgcttaatttctaaataaattcttacccatttactgacagtgaaaattagacaactttaaaagagattctattcatctttaaattttgattactaggtttttttgggttttcacatatgaactcccaatttaaccccccattatattagcatatatggtaggagaggtatattaattttcatacaaaagcaatggtttcatgtgctatgtgttgcttgttaaaacacagtgcaggacgaggccacttatttacagctggggcacagcttattttcagcttatttataactgaagggtatgcacactgtgagtacacttatttgacgacattactgtagttcttaaggtgatacaataaaagagatttttaagaacagatagtaggaactacttttcaaatgctcttgaagactgttactctctaaaattccagtcaacctttaaacacttctagaacttttattttaatgaaaatagcttgttttagtttgaagctataaagaacaagttagatagttttaaaacaagcacatgtctttattaagtgacagaaaataattcaacatttttaaaagtaaaaaggacattttagcatacaaacatttctcaagaaatttaataagtgtatattcctctaacaacagagtacatttatcaagtttttattctccagtgttcttaagtttcctgctgtgaatttagaagtaaaacaaaatattcaggaaattgatatgcaacaaaaactttattattgctatttgttaaaatctcaatctactaagtaaaatattttctcactattaaatccaatagatcacatagaaaataaacagatttttattttactatttatattcctatcatatttaaatgctaaataaaacttacctctacaattctcaattcctataattctaactctgctgcttatgacaaacacagctgtctcctacacaacattcttgtcatgtaaaaacatttttagtatgtgctttaactccaaggaacactggtctctcaataattttgtaaccctaaaaatattttttctttgatattatacaaattacacttggtgccaagtttatttgtcatatttctgctatttttatttatctatccactgtgttgctgggggactagggaagagagtgagagagagagagagagagagagaaagagagagagagagagagagagtgtcattgcatgtctgtgaaggtcagaagagtttgtaggagtcagttctttcttcccaccatgtaggtctcaggagtcaagcttgggtcgtcaggcttggcagcaagtgccttacctactgagccctttccccggtccctgaaggtttaccttaatgcctatccaatagcattctcaaacttgaacagtgtgtgcaaaagtttttttcagtggagtaaggggtaggtaatgagtttctttccaccagaatttccttatatcacaattagttacaaaaaaaaggaagcaacttctagaaaaaaatataaattctgctatagaaagcatctttagcagattaagcagatgctgcctgtagttaaaattttcaatataaagcacagtttctacatagctttatggtgacattgaagtattttgtttataaaggtacatgctttaaaagtaagcacaaaaaaggataaaatcaatgaagaaatgtaacagatacactaaaaaagaatgagatgatttgctttgccaaaaggagttgccgcagtgtcatttcctaaagcaaatcaaaattgtttttagaatattttagtagacatagaggaaaattgctgcatttgggtcattattaacaaatattgtttcttaatctcaccaatttatcaagacataatatttgcattggctatagaattcaaagtatatattaaaacatcaagtgaggatgattaaagtatagatcactgctataaattacacagcataagcacattttttctgaagaacattttcctaagtgcacttacacacagacactccctcttaattctttgtatgcatctctacatcatcatttggagaacagattttaaaacaagtgagctctgtgaagttcacagagctacaagcaaataggagaccttgtgggagaggccatttgcacctctgatcccgttcagtgatgagcatggttaagatacatcatcatcctgacaaggtacagaaaaagaaaagcaaatgcaaaatgctgtgaatggtcattttctgagaaaatgaggatgcaaactactgctaactaaaaagcattcctctgtttcatttaaatccatcaagtgttggatctgtactattctgtatgatattctaaacattatcttcaaagtcaggcataaagttactgatgtccttagtttttaagcagtaaattctagtcagcaaagttcaaaataatcacatagacatgaacataccagaatcaaattaaaatatacaatcattaagtttgctctattgataaaaaatatacctccaatccatttaagcttgttatctaaaaatcagactgatttttccataactctgaacaaaggcttcaacactctattgcatccctacagtgcatcaatttcatttcttacctgctcactctacccaaggtcactgttttcaaggttcctggataaaagaatcttgcattgttatttacacaatgaatataattttcatctaatataggaattcacacattgatcagaccaacaaaaacaaaaatttagagttgggaatgaaaaatatagtctttctaaattatagcagcatggctgataagcagcagtctctttggacattccaagagtgtgtagtacttcagtttccttacccaacacaattctgaagcatgcattaccaaataaacacatgaaaataaagccttcttgggtattctaatacccatggagggatttacagagacaaagtttggagctgagacagaaggaaggaccatccagagactgtcccacccggggatccatcccataaacagccaccaaacccagacactattgcatatgccagcaagattttgctgaagggacactgatatagctgtctcttgtgaagctatgccagtacctggcaaatacaaaagtggatgttcacagtcatctattggatagaacatagggcccccaatggaggagctagagaaagtacccaaggagctaaaggggtctgcaaccctgtaggaggaacaacaatatgaactaaccaatacccccagagctcgtgtctctagctgcatatgtagcagaagatggcctagtcagccatcattgggaaaagaggcccttggtcttatgaagattatatgccccagtataggggaatgccagagccaggagcaggagtgggtgggttggagagcagagcggcgggagggtaaagggactttcgggatagcatttgaaatgtaaatgaagaaaatatctaataaaaaaaatctttttaaagaaatgaggaaaaaaagaaaggaaggaaagccttcatgtgtatgctctgccccatcagtagagcttacaggcaactgaggacctctagtgcttcagagcacgtactacatcttcagggctttccatgaattgggtgcatcactcactcctctacatgaacaggggtttatttgctaagtgcccctcaattttccttagaacttcccaaatactgtctccctacagcctgtttaaaaaaaaaaaaaacagactctgaagcttcagtataaggctaaaaatcactttcatggaggcacaggagccttgtaatcccaggcaaaagcaggactgcagagttcaagactaaccagtgtgacacacagtaagtttaagaccagcctagactacacagtccaaaatcggggctgtgttaattcaaataaatatgttcaatttcacataagtatacatcaataaaaataaacctacggacgaaaatatcaattgccatgagctaatttcagagggaaatttttaacagaattttttaattggtccaatttcattctgtttacacaatttttatcacaactagacagaagcaggtgaagagactctgacgcttcagtctgcacaacaaagctgggaccaaaggaacttgccagcttacaattctgcagtcttaaaggggcttcatcccagtgagctttcctgcgtaacaggttgatctaacttttttgtttttgtttttgtttttttgttttttttgttttggtttggtttggtttggttttggtttggtttggtttggtttttggtttttcgagacagggtttctctgtgtagccctggctgtcctggaactcactttgtagaccaggctggtcttgaactcagaaattcgcctgcctctgcctcccgactgttgggattaaaggaatgcaccaccacgcccggctccttcagggttttatacagataacttctacccctacccaacaggttcctaacaaaggatatgactttttttttttttttttttttttttttttttggctaggacctcactgtctagccctggttgccctggaacttgctatgtagagcagactggctgaaactcagagctcagcctaactctgctctccgtctggagtgataagacctaaggcaagagccgccacacctgacagtatggtactgtaggattactgccgtctgtagctttcctcttcctcctcctactaaaacaaaaatgatacttctgaacttaatttcctaaattccgatttattctctttaaacagagagagagaagataatacaaggtcccactaggaaaagtaaagaattgctggtagcgatcaatccttccaaaagtgagaacagatcccacacacatagaacacaaaacttgggttctcttctgcttcttacataactaaaatacgggatctcatatttcccacaaccaaatatacattgtatttccagcactctggtacaaaaatcagaaagattaaacttcaaattagtctgtaagtaaagaggctctgtgtctccagtaaacacccaatgaacatatacagtcatctttttaaacctgtagctaattttctgttaacacgctttattttgttgtatttactatcaaagctttgaaccttacatattcagccatagtttagaagttttgaaagaaggccagacacaaacccacccctggtagccaagattagctgaaaactggcactgtgatagtttaaaacgatagtccttactgtgccttctgagtttggaagtctttagtacttaacatcttcttaaagtgtttaaaataacattcaccaagaggctggatctgggaggagtccggaggatgtatatatgatcaaatacactgaatgattctacaaatctataaatcataaaagagaatggcaagtggctagaatctgctttttatttcatatttttcacttaactattttattttggactaaaactaggatccaagatatgttactatatgcatttctttttacaagattatttctgaaacattaatagtaaatgttaaaatcaattgtgtttacagagaaacagagctttaaaaaaagtaaccaagaaaatagccccaaagaagcacctcataaatcaagtactagccgccaaagccgtggctctccccgctcacccaggacagtgagacgctcacacatgagagacagaagagaggccttggggactggagaaggggatgggatttaaaatacagctgaggaacgagctgctgcaaagctgtgcatatctacccagagaacgggtttaggtatgaacctaatctgttgactattttttacatcccaggtctcctaaaagaagacagctcacatttggctgcctgttgtcaactttttatctttctcttgaccacttagaaaaacttgtctccctacttttgccttcctggcataatggaaaaagtgcaatacctcactcgctcggctatcaggagagcctccactattgaaatgcctcagcaagcacgtcagaatctccagaacctatttatcaatttctgcctcatcttgatatgtctgctgctgatctgcatcattgtgatgcttctgtgaagagctgccgccactccagacctgcaacatgccaactcagcttaaagccgagcactccgtcatgggggaagcaacgcaccgctgccttccggctgagaacagctttgtgagggtcacgattaagagtgaaactaattgttagccagtgtattcatctgctggatcttgtaaacaaactttacagtggctaagtgtgcataatgtagctgccaatttcctcaacatggctcataaatttctttcctaaatatttctgacaatgaagtatgagttttaattttgaaacaccactaccaaaaaagttcactttctatatccaactctagcaaggtcactttatagctccagtctggggaataaatggaatatatactgcattgccactaccttttttataggaactaagtgttcttcagttctccacaaagtagaaaatagtcatttaaatgaagtgttcttattgaaaatagctcactgggtcctcacatagatgacacaatgatgacgatgatgatgatgacgacgatgatgatgatgacgacgattcaaatctcttggtcactactgtcagaacaaataccttgaaaaaatgttgtgccctttttctacacactagtatttttaagcttatgagagttaaagggggaaatgtaagaacatattctcatataaaattattcactaattttgctcaagggctgtgtacatgtacatgtacattaaccacaaatctgctctctctagggtttgatcaacaagcaggaacatcttgctgtgttggctgtgataatcactaaagatccagagtctcacctgaagcttgtcgctctcatgtatctccttggtttaatagcgtttctcaaataatgaagataacagtaataaccaagctgtagtaagacacagctctttcttttcctgatctcccagagaggaagtggttgtcccctaactttagaagtagaactagagagctagtgtataattcttactgaccatggtttcaatttccctttctaagtttctactatctctcaataactactaaaatagaagcaaaaaatagattggaaaatatattcaccaaaatttagtgacttaaactggatcaagtacaaaacagtttatgcctaactttgcaaacccacaatttttaagtccagttcaaaacatgctactaacatagcaattattaaggaagagaattgtaattagatatattttccataaccattactacaaagacataaaacatcagtattcactttggtctttagcagagcagctgaaggctgctgatttaaactgtttgaacccctggtagaaaggaagctgttcgggttcagaaattatctaaagagtagcccctaaaggagtccaatggtaacagggggtctcttcataaaggctggaggggcccatcacagaaactataaaacatggatgacatcatctctaactcagtggtatttaacgttccctttctgacgctgtggcccttttatatagttcctcatgttgtggtgatacccaacaataaaatcatttttgttgctatttcataactgtaatttcgcctccttacctccactatgaattgtaaatattttggaaacaggtttgcatgaggggttgcaacccacaggttgagaaccactggtaagaggtctgtaaaaaatacataagttgtgtagttggccatgctgagagaaacgtgacgctgaagctgaaaagcaccaggcatttgttatattgctcactaccacatcaacttcaaggtataaactgtcagctcataaggctcttctcattaaacatttaatgctgccttttacatttttagtagtttatttgtagtttatctgtaactgtttatctgtaaaatgaaattggaatgacctatattcaaatggagttttgattttggtgaaattactatacaattatattctattgactataaaatgttgtcttgttacaaagagttttgtgttataaagggcagagtgggggaacttttaccaaacaaagtaaaaatcaagatttccagagaaaaacacaaaagaggataggcctacacataataatattttttagcctccaattattttgagtcttctggcaaagtaggagtgcaatcagtaacataagaaggcttggttacacactaataccagatttttaatgtcttgaaaggacatgataaattatatagacatggaaatagagatagacaaatcaatccgtaaagcatgtatccactagcagatgctttaagctacccactatgtcacaaactgtgactgcatgtgtcatagtcacctcctcagtatacagattgtctcagctaaagagttaaagataaaacagcatgaaaaataatgaaactaagaaaattttctttttaaaaaaacataagaaaatccgagttacaagccagactttttcattcacaattcatttcgaagaagtcttttatgttatttaatcaaaatcttcatttaaataacagaaagtctctgctaacatgagttagcaatcccttaactgatcaaaccactttatgatttatatacatacaaaatttcatctccatttttaacaactacagtccagggaacatgagacaatgcaaagttagcaatagagttccctacttaaccaccagggggcagacccactgtgggcttacgagaagctaatggttattttatgtgttgaggaggtctacattctcacacccctccttgaaaacaaggagtacagaagagcacatttcaatgaaacagtccctcctgaacaaggccgagactatcacaaccctacagagacaatttggggtactatgggtgaggtatcaaaacgcaaagaaaagagccaagactgtggtgaactataacgtgaaccaaacaaaggcaaatcgtttttcttcaaccacaaaggaaaaatggttcagaatattacacaggatggtgcacagtggttctcaacctccctgatgctgtgaccctgtaatagtctcttgtgttgtggtgacccccaaccatataattttgctccttttatgaatcataatgtgtctgttttccaatagtcttaggtgactccatcagccgttccaccgctcagagtggttgggcccctcgggctgagaatcactgctctaagtcataggggagcttcccattagctgtcttctccctccctctactgtgttctacaaagattcacacctgagtggttgctccttagtgcatatcttacaaagaaatttccctattagcatgttattccacagaccttatagggattaatacaccatgtttctctgtatgtggttattatcataaagtagttcctttaaactaattcaataaatcttggtgttccattcatttcttgaaaactgcagctgatttatatgctcatatacaaagaaagcattatcaaaatagtacaaggaactgggttccaccttctcattttatagaaggcccaaagaaattaagtgccaggtcaaaagacacctgagttctcaccagactaaattcccaaagagtctactaaaagtcatagaaatgagtacagtcccatgtttactaacattcatagcaactagaaataaaaccagcctagagatccatccacctgtaagcatgtaagaaaatgtggtacatatacacgatgcaactttatttggccataaaggaaaattaaattgtgacttctgcagaaaaccaaatgggacttgaaatcactacattaatcaaagcatgccagacccgggaaggcaaatgcgtcatgttttctctcatattgttaatctagatttgaaatcttgttcatgtgaattcttgttctacttgtgaaactagaaagagctaagctagagaagaataaagagggaggcagagtatagagagggtgtgtaaggagggggacagagcaaagggctcttgtgggaaagggaggggcagtagggtggaaccaggctgagggcaagcagaggaccaagaagacgactgtatgaaaatcctataatgaaataaacactttgaatgcagattaaaaaataaatgaaaaactaacttatgaagaaaaagaaaaaaaatatccaagctctaaatgtgacatgatcagaacaaagtccacggcctgtttgttccaatcagtttccttcctatgataccgtgacagcttctgtctgttttccgtttctttcaactgaatgtgtttaatttctatgcctacttaacaagagagaaagtaaaggccaagttaagtacaatgtttaaggctatcaaattaattaatctcaaaagatttgtaagtttttctaaacacagtgtgtcaattttatagcacttaacggtttttttaacttgtgtacttgctgcaatacataaaacatccactggagggaagcaaacactaggttttcagccttacctgcattggcgtctcacttccttgctgaaactcttcagtttgtggctgttgccaaggagctaaactgtaagaatcgtctgtggttctgccttccaatgggtttgtccgtaactgctctgtcagccacatctgagtcctcacagaaggttgaatgggaactccacctactgcctgctggcccagcattaaatactttggagactcaaaagtctctgagcctgtcacaatgggcttgctctcaggtaaggcactgtatgtcatgtctaaagtctgtctcctgagagcagaggaagagactctgacatcttctttgttgcagctctcaaatttatatttgcagtccagagttgatgaccgctttttatttatttccttgtcttccagcttaagcagctccatactgtcatgtaagcaacttggcccaaaggtccgtaagtgggacataaaaggcttgcccttccctgtcatctccaagctattcctcagctcttgcgggtatgcaatgccactgtggttgcgggtatctggg

SEQ ID NO: 1053 - DNA, 21 bp, synthetic construct
  source 1..21: mol_type = other DNA
tctgctctctctagggtttga

SEQ ID NO: 1054 - DNA, 22 bp, synthetic construct
  source 1..22: mol_type = other DNA
ggagatacatgagagcgacaag

SEQ ID NO: 1055 - DNA, 24 bp, synthetic construct
  source 1..24: mol_type = other DNA
acagccaacacagcaagatgttcc

SEQ ID NO: 1056 - DNA, 25 bp, synthetic construct
  source 1..25: mol_type = other DNA
cacccgtaagacttcatacaacaca

SEQ ID NO: 1057 - DNA, 25 bp, synthetic construct
  source 1..25: mol_type = other DNA
tggcagccaaatatgagataactgt

SEQ ID NO: 1058 - DNA, 16 bp, synthetic construct
  source 1..16: mol_type = other DNA
tgccaaggctacctaa

SEQ ID NO: 1059 - DNA, 20 bp, synthetic construct
  source 1..20: mol_type = other DNA
ggcaaattcaacggcacagt

SEQ ID NO: 1060 - DNA, 20 bp, synthetic construct
  source 1..20: mol_type = other DNA
gggtctcgctcctggaagat

SEQ ID NO: 1061 - DNA, 27 bp, synthetic construct
  source 1..27: mol_type = other DNA
aaggccgagaatgggaagcttgtcatc

SEQ ID NO: 1062 - protein, 13 aa, synthetic construct
CSPDAHLGCISYC

SEQ ID NO: 1063 - protein, 13 aa, synthetic construct
CSPDAYLGCISYC

SEQ ID NO: 1064 - protein, 13 aa, synthetic construct
  SITE 3: note = 4Hyp
CPXDAYLGCISYC

SEQ ID NO: 1065 - protein, 13 aa, synthetic construct
  SITE 3: note = 4Hyp
CSXDAHLGCISYC

SEQ ID NO: 1066 - protein, 13 aa, synthetic construct
  SITE 3: note = azetidine
CSXDAHLGCISYC

SEQ ID NO: 1067 - protein, 13 aa, synthetic construct
  SITE 3: note = 4Hyp
  SITE 10: note = t-butyl glycine
CPXDAYLGCXSYC

SEQ ID NO: 1068 - protein, 13 aa, synthetic construct
  SITE 2: note = 6-azido lysine
CXPDAHLGCISYC

SEQ ID NO: 1069 - protein, 13 aa, synthetic construct
  SITE 3: note = 6-azido lysine
CSXDAHLGCISYC

SEQ ID NO: 1070 - protein, 13 aa, synthetic construct
  SITE 5: note = 6-azido lysine
CSPDXHLGCISYC

SEQ ID NO: 1071 - protein, 15 aa, synthetic construct
ACSADDWLGCISWCA

SEQ ID NO: 1072 - protein, 22 aa, synthetic construct
  SITE 16..21: note = MeGly
  SITE 22: note = 6-fluorescein lysine
ACSADDWLGCISWCAXXXXXXX

SEQ ID NO: 1073 - protein, 15 aa, synthetic construct
ACSSDAYLGCISWCA

SEQ ID NO: 1074 - protein, 22 aa, synthetic construct
  SITE 16..21: note = MeGly
  SITE 22: note = 6-fluorescein lysine
ACSSDAYLGCISWCAXXXXXXX

SEQ ID NO: 1075 - protein, 15 aa, synthetic construct
ACPPDAHLGCISWCA

SEQ ID NO: 1076 - protein, 13 aa, synthetic construct
CPPDAHLGCISWC

SEQ ID NO: 1077 - protein, 15 aa, synthetic construct
ACPQDAYLGCISWCA

SEQ ID NO: 1078 - protein, 13 aa, synthetic construct
CPQDAYLGCISWC

SEQ ID NO: 1079 - protein, 15 aa, synthetic construct
ACPPDSWQGCISYCA

SEQ ID NO: 1080 - protein, 22 aa, synthetic construct
  SITE 16..21: note = MeGly
  SITE 22: note = 6-fluorescein lysine
ACPPDSWQGCISYCAXXXXXXX

SEQ ID NO: 1081 - protein, 15 aa, synthetic construct
ACSPDAHLGCISYCA

SEQ ID NO: 1082 - protein, 22 aa, synthetic construct
  SITE 16..21: note = MeGly
  SITE 22: note = 6-fluorescein lysine
ACSPDAHLGCISYCAXXXXXXX

SEQ ID NO: 1083 - protein, 20 aa, synthetic construct
  SITE 14..19: note = MeGly
  SITE 20: note = 6-fluorescein lysine
CSPDAHLGCISYCXXXXXXX

SEQ ID NO: 1084 - protein, 21 aa, synthetic construct
  SITE 15..20: note = MeGly
  SITE 21: note = 6-fluorescein lysine
CSPDAHLGCISYCAXXXXXXX

SEQ ID NO: 1085 - protein, 20 aa, synthetic construct
  SITE 14..19: note = MeGly
  SITE 20: note = 6-fluorescein lysine
CSPDAHLGCISYCXXXXXXX

SEQ ID NO: 1086 - protein, 21 aa, synthetic construct
  SITE 1: note = N-Fluorescein glycine
  SITE 2..6: note = MeGly
GXXXXXACSPDAHLGCISYCA

SEQ ID NO: 1087 - protein, 16 aa, synthetic construct
  SITE 2: note = 1-Naphthylalanine
NXNCSPDAHLGCISYC

SEQ ID NO: 1088 - protein, 16 aa, synthetic construct
  SITE 15: note = Pipecolic Acid
CSPDAHLGCISYCEXW

SEQ ID NO: 1089 - protein, 16 aa, synthetic construct
CSPDAHLGCISYCEPW

SEQ ID NO: 1090 - protein, 16 aa, synthetic construct
NWNCSPDAHLGCISYC

SEQ ID NO: 1091 - protein, 17 aa, synthetic construct
NWNCSPDAHLGCISYCA

SEQ ID NO: 1092 - protein, 16 aa, synthetic construct
  SITE 15: note = azetidine
CSPDAHLGCISYCEXW

SEQ ID NO: 1093 - protein, 16 aa, synthetic construct
NWNCSPDAHLGCISYC

SEQ ID NO: 1094 - protein, 16 aa, synthetic construct
  SITE 15: note = D-Proline
CSPDAHLGCISYCEXW

SEQ ID NO: 1095 - protein, 16 aa, synthetic construct
  SITE 2: note = 1-Naphthylalanine
NXNCSPDAHLGCISYC

SEQ ID NO: 1096 - protein, 16 aa, synthetic construct
  SITE 2: note = D-tryptophan
NXNCSPDAHLGCISYC

SEQ ID NO: 1097 - protein, 16 aa, synthetic construct
  SITE 2: note = D-tryptophan
NXNCSPDAHLGCISYC

SEQ ID NO: 1098 - protein, 17 aa, synthetic construct
HWMCSPDAHLGCISYCA

SEQ ID NO: 1099 - protein, 17 aa, synthetic construct
ACSPDAHLGCISYCPHP

SEQ ID NO: 1100 - protein, 17 aa, synthetic construct
ACSPDAHLGCISYCEPW

SEQ ID NO: 1101 - protein, 17 aa, synthetic construct
NEVCSPDAHLGCISYCA

SEQ ID NO: 1102 - protein, 18 aa, synthetic construct
ACSPDAHLGCISYCPIVH

SEQ ID NO: 1103 - protein, 13 aa, synthetic construct
CSPDAHLGCISYC

SEQ ID NO: 1104 - protein, 17 aa, synthetic construct
HTSCSPDAHLGCISYCA

SEQ ID NO: 1105 - protein, 16 aa, synthetic construct
  SITE 2: note = N-methyl tryptophan
NXNCSPDAHLGCISYC

SEQ ID NO: 1106 - protein, 16 aa, synthetic construct
  SITE 2: note = N-methyl tryptophan
NXNCSPDAHLGCISYC

SEQ ID NO: 1107 - protein, 15 aa, synthetic construct
ACSPDAHLGCISYCA

SEQ ID NO: 1108 - protein, 18 aa, synthetic construct
ACSPDAHLGCISYCEHQE

SEQ ID NO: 1109 - protein, 17 aa, synthetic construct
ESFCSPDAHLGCISYCA

SEQ ID NO: 1110 - protein, 17 aa, synthetic construct
  SITE 17: note = 6-azido lysine
NWNCSPDAHLGCISYCX

SEQ ID NO: 1111 - protein, 17 aa, synthetic construct
  SITE 17: note = 6-azido lysine
NWNCSPDAHLGCISYCX

SEQ ID NO: 1112 - protein, 16 aa, synthetic construct
NWNCSPDAHLGCISYC

SEQ ID NO: 1113 - protein, 17 aa, synthetic construct
  SITE 17: note = 6-azido lysine
CSPDAHLGCISYCEPWX

SEQ ID NO: 1114 - protein, 16 aa, synthetic construct
CSPDAHLGCISYCEPW

SEQ ID NO: 1115 - protein, 14 aa, synthetic construct
  SITE 14: note = 6-azido lysine
CSPDAHLGCISYCX

SEQ ID NO: 1116 - protein, 13 aa, synthetic construct
CSPDAHLGCISYC

SEQ ID NO: 1117 - protein, 17 aa, synthetic construct
  SITE 2: note = D-tyrosine
  SITE 17: note = 6-azido lysine
NXNCSPDAHLGCISYCX

SEQ ID NO: 1118 - protein, 17 aa, synthetic construct
  SITE 15: note = D-Proline
  SITE 17: note = 6-azido lysine
CSPDAHLGCISYCEXWX

SEQ ID NO: 1119 - protein, 17 aa, synthetic construct
  SITE 15: note = azetidine
  SITE 17: note = 6-azido lysine
CSPDAHLGCISYCEXWX

SEQ ID NO: 1120 - protein, 17 aa, synthetic construct
  SITE 15: note = Pipecolic Acid
  SITE 17: note = 6-azido lysine
CSPDAHLGCISYCEXWX

SEQ ID NO: 1121 - protein, 14 aa, synthetic construct
  SITE 14: note = K(N3)(PYA-Maleimide)
CSPDAHLGCISYCX

SEQ ID NO: 1122 - protein, 16 aa, synthetic construct
CSPDAHLGCISYCEPW

SEQ ID NO: 1123 - protein, 16 aa, synthetic construct
CSPDAHLGCISYCEPW

SEQ ID NO: 1124 - protein, 23 aa, synthetic construct
  SITE 23: note = 6-azido lysine
CSPDAHLGCISYCEPWGGSGGSX

SEQ ID NO: 1125 - protein, 15 aa, synthetic construct
ACPGDAHLGCISYCA

SEQ ID NO: 1126 - protein, 13 aa, synthetic construct
CPGDAHLGCISYC

SEQ ID NO: 1127 - protein, 15 aa, synthetic construct
ACPPDSHLGCISYCA

SEQ ID NO: 1128 - protein, 13 aa, synthetic construct
CPPDSHLGCISYC

SEQ ID NO: 1129 - protein, 15 aa, synthetic construct
ACSADDWLGCISYCA

SEQ ID NO: 1130 - protein, 13 aa, synthetic construct
CSADDWLGCISYC

SEQ ID NO: 1131 - protein, 13 aa, synthetic construct
  SITE 3: note = 4Hyp
  SITE 10: note = t-butyl glycine
CPXDAYLGCXSYC

SEQ ID NO: 1132 - protein, 16 aa, synthetic construct
  SITE 3: note = 4Hyp
  SITE 10: note = t-butyl glycine
CPXDAYLGCXSYCEPW

SEQ ID NO: 1133 - protein, 16 aa, synthetic construct
  SITE 6: note = 4Hyp
  SITE 13: note = t-butyl glycine
NWNCPXDAYLGCXSYC

SEQ ID NO: 1134 - protein, 15 aa, synthetic construct
  SITE 4: note = 4Hyp
  SITE 11: note = t-butyl glycine
ACPXDAYLGCXSYCA

SEQ ID NO: 1135 - protein, 16 aa, synthetic construct
  SITE 6: note = 4Hyp
  SITE 13: note = t-butyl glycine
NWNCPXDAYLGCXSYC

SEQ ID NO: 1136 - protein, 14 aa, synthetic construct
  SITE 3: note = 4Hyp
  SITE 10: note = t-butyl glycine
  SITE 14: note = 6-azido lysine
CPXDAYLGCXSYCX

SEQ ID NO: 1137 - protein, 17 aa, synthetic construct
  SITE 6: note = 4Hyp
  SITE 13: note = t-butyl glycine
  SITE 17: note = 6-azido lysine
NWNCPXDAYLGCXSYCX

SEQ ID NO: 1138 - protein, 13 aa, synthetic construct
  SITE 3: note = 4Hyp
CPXDAYLGCISYC

SEQ ID NO: 1139 - protein, 14 aa, synthetic construct
  SITE 3: note = 4Hyp
  SITE 14: note = 6-azido lysine
CPXDAYLGCISYCX

SEQ ID NO: 1140 - protein, 13 aa, synthetic construct
  SITE 3: note = 4Hyp
CPXDAYLGCISYC

SEQ ID NO: 1141 - protein, 13 aa, synthetic construct
  SITE 3: note = 4Hyp
CSXDAHLGCISYC

SEQ ID NO: 1142 - protein, 15 aa, synthetic construct
  SITE 4: note = 4Hyp
ACSXDAHLGCISYCA

SEQ ID NO: 1143 - protein, 14 aa, synthetic construct
  SITE 3: note = 4Hyp
  SITE 14: note = 6-azido lysine
CSXDAHLGCISYCX

SEQ ID NO: 1144 - protein, 13 aa, synthetic construct
  SITE 3: note = 4Hyp
CSXDAHLGCISYC

SEQ ID NO: 1145 - protein, 15 aa, synthetic construct
  SITE 4: note = Aib
  SITE 11: note = t-butyl glycine
ACPXDAHLGCXSYCA

SEQ ID NO: 1146 - protein, 18 aa, synthetic construct
TYMNCPPDAHLGCISYCA

SEQ ID NO: 1147 - protein, 15 aa, synthetic construct
ACPPDAHLGCISYCA

SEQ ID NO: 1148 - protein, 15 aa, synthetic construct
  SITE 4: note = Aib
  SITE 11: note = t-butyl glycine
ACPXDAYLGCXSYCA

SEQ ID NO: 1149 - protein, 13 aa, synthetic construct
CSADAHLGCISYC

SEQ ID NO: 1150 - protein, 15 aa, synthetic construct
  SITE 4: note = Aib
  SITE 11: note = t-butyl glycine
ACSXDAHLGCXSYCA

SEQ ID NO: 1151 - protein, 15 aa, synthetic construct
  SITE 11: note = 2-amino-3-ethyl-pentanoic acid
ACSPDAHLGCXSYCA

SEQ ID NO: 1152 - protein, 15 aa, synthetic construct
  SITE 11: note = t-butyl glycine
ACPPDAYLGCXSYCA

SEQ ID NO: 1153 - protein, 15 aa, synthetic construct
  SITE 4: note = Aib
  SITE 11: note = t-butyl glycine
ACSXDAYLGCXSYCA

SEQ ID NO: 1154 - protein, 13 aa, synthetic construct
CAPDAHLGCISYC

SEQ ID NO: 1155 - protein, 15 aa, synthetic construct
  SITE 4: note = Aib
ACPXDAHLGCISYCA

SEQ ID NO: 1156 - protein, 15 aa, synthetic construct
  SITE 11: note = t-butyl glycine
ACSPDAYLGCXSYCA

SEQ ID NO: 1157 - protein, 15 aa, synthetic construct
  SITE 11: note = t-butyl glycine
ACSPDAHLGCXSYCA

SEQ ID NO: 1158 - protein, 18 aa, synthetic construct
IDSNCPNDAHLGCISYCA

SEQ ID NO: 1159 - protein, 18 aa, synthetic construct
WGKSCPIDAHLGCISYCA

SEQ ID NO: 1160 - protein, 15 aa, synthetic construct
ACSPDAYLGCISYCA

SEQ ID NO: 1161 - protein, 15 aa, synthetic construct
ACPPDAYLGCISYCA

SEQ ID NO: 1162 - protein, 15 aa, synthetic construct
  SITE 4: note = Aib
ACSXDAHLGCISYCA

SEQ ID NO: 1163 - protein, 15 aa, synthetic construct
  SITE 11: note = cyclohexyl glycine
ACSPDAHLGCXSYCA

SEQ ID NO: 1164 - protein, 22 aa, synthetic construct
  SITE 16..21: note = MeGly
  SITE 22: note = 6-fluorescein lysine
ACAPDAHLGCISYCAXXXXXXX

SEQ ID NO: 1165 - protein, 15 aa, synthetic construct
  SITE 8: note = t-butyl alanine
ACYLPDWXCGDEYCA

SEQ ID NO: 1166 - protein, 15 aa, synthetic construct
  SITE 13: note = 2-Naphthylalanine
ACSPDAHLGCISXCA

SEQ ID NO: 1167 - protein, 15 aa, synthetic construct
  SITE 13: note = 3-t-Butyl-Tyrosine
ACSPDAHLGCISXCA

SEQ ID NO: 1168 - protein, 15 aa, synthetic construct
  SITE 6: note = Aib
ACSPDXHLGCISYCA

SEQ ID NO: 1169 - protein, 15 aa, synthetic construct
  SITE 13: note = 1-Naphthylalanine
ACSPDAHLGCISXCA

SEQ ID NO: 1170 - protein, 15 aa, synthetic construct
  SITE 8: note = t-butyl alanine
ACSPDAHXGCISYCA

SEQ ID NO: 1171 - protein, 15 aa, synthetic construct
  SITE 8: note = beta-cyclobutylalanine
ACSPDAHXGCISYCA

SEQ ID NO: 1172 - protein, 15 aa, synthetic construct
ACSPDAHLGCISWCA

SEQ ID NO: 1173 - protein, 15 aa, synthetic construct
  SITE 6: note = Abu
ACSPDXHLGCISYCA

SEQ ID NO: 1174 - protein, 15 aa, synthetic construct
  SITE 4: note = azetidine
ACSXDAHLGCISYCA

SEQ ID NO: 1175 - protein, 13 aa, synthetic construct
  SITE 3: note = azetidine
CSXDAHLGCISYC

SEQ ID NO: 1176 - protein, 14 aa, synthetic construct
  SITE 3: note = azetidine
  SITE 14: note = 6-azido lysine
CSXDAHLGCISYCX

SEQ ID NO: 1177 - protein, 13 aa, synthetic construct
  SITE 3: note = azetidine
CSXDAHLGCISYC

SEQ ID NO: 1178 - protein, 15 aa, synthetic construct
ACSPDDHLGCISYCA

SEQ ID NO: 1179 - protein, 15 aa, synthetic construct
ACSPDSHLGCISYCA

SEQ ID NO: 1180 - protein, 15 aa, synthetic construct
  SITE 8: note = Abu
ACSPDAHXGCISYCA

SEQ ID NO: 1181 - protein, 15 aa, synthetic construct
  SITE 13: note = 4-pyridylalanine
ACSPDAHLGCISXCA

SEQ ID NO: 1182 - protein, 15 aa, synthetic construct
  SITE 4: note = D-Alanine
ACPXDAHLGCISYCA

SEQ ID NO: 1183 - protein, 15 aa, synthetic construct
  SITE 11: note = t-butyl alanine
ACSPDAYLGCXSYCA

SEQ ID NO: 1184 - protein, 15 aa, synthetic construct
  SITE 11: note = cyclopentyl glycine
ACSPDAHLGCXSYCA

SEQ ID NO: 1185 - protein, 15 aa, synthetic construct
  SITE 11: note = cyclobutyl glycine
ACSPDAHLGCXSYCA

SEQ ID NO: 1186 - protein, 15 aa, synthetic construct
  SITE 9: note = D-alanine
ACSPDAHLXCISYCA

SEQ ID NO: 1187 - protein, 15 aa, synthetic construct
  SITE 8: note = Aib
ACSPDAHXGCISYCA

SEQ ID NO: 1188 - protein, 15 aa, synthetic construct
  SITE 11: note = cyclopropyl glycine
ACSPDAHLGCXSYCA

SEQ ID NO: 1189 - protein, 15 aa, synthetic construct
  SITE 11: note = beta-methyl isoleucine
ACSPDAHLGCXSYCA

SEQ ID NO: 1190 - protein, 22 aa, synthetic construct
  SITE 16..21: note = MeGly
  SITE 22: note = 6-fluorescein lysine
ACSADAHLGCISYCAXXXXXXX

SEQ ID NO: 1191 - protein, 22 aa, synthetic construct
  SITE 16..21: note = MeGly
  SITE 22: note = 6-fluorescein lysine
ACSPAAHLGCISYCAXXXXXXX

SEQ ID NO: 1192 - protein, 22 aa, synthetic construct
  SITE 16..21: note = MeGly
  SITE 22: note = 6-fluorescein lysine
ACSPDAALGCISYCAXXXXXXX

SEQ ID NO: 1193 - protein, 22 aa, synthetic construct
  SITE 16..21: note = MeGly
  SITE 22: note = 6-fluorescein lysine
ACSPDAHAGCISYCAXXXXXXX

SEQ ID NO: 1194 - protein, 22 aa, synthetic construct
  SITE 16..21: note = MeGly
  SITE 22: note = 6-fluorescein lysine
ACSPDAHLACISYCAXXXXXXX

SEQ ID NO: 1195 - protein, 22 aa, synthetic construct
  SITE 16..21: note = MeGly
  SITE 22: note = 6-fluorescein lysine
ACSPDAHLGCASYCAXXXXXXX

SEQ ID NO: 1196 - protein, 22 aa, synthetic construct
  SITE 16..21: note = MeGly
  SITE 22: note = 6-fluorescein lysine
ACSPDAHLGCIAYCAXXXXXXX

SEQ ID NO: 1197 - protein, 22 aa, synthetic construct
  SITE 16..21: note = MeGly
  SITE 22: note = 6-fluorescein lysine
ACSPDAHLGCISACAXXXXXXX

SEQ ID NO: 1198 - protein, 13 aa, synthetic construct
  SITE 2: note = 6-azido lysine
CXPDAHLGCISYC

SEQ ID NO: 1199 - protein, 13 aa, synthetic construct
  SITE 3: note = 6-azido lysine
CSXDAHLGCISYC

SEQ ID NO: 1200 - protein, 13 aa, synthetic construct
  SITE 5: note = 6-azido lysine
CSPDXHLGCISYC

SEQ ID NO: 1201 - protein, 16 aa, synthetic construct
  SITE 2..3: note = 4Hyp
  SITE 10: note = t-butyl glycine
CXXDAYLGCXSYCEPW

SEQ ID NO: 1202 - protein, 17 aa, synthetic construct
  SITE 3: note = 4Hyp
  SITE 10: note = t-butyl glycine
CPXDAYLGCXSYCEPWK

SEQ ID NO: 1203 - protein, 17 aa, synthetic construct
  SITE 3: note = 4Hyp
  SITE 10: note = t-butyl glycine
CPXDAYLGCXSYCEPWC

SEQ ID NO: 1204 - protein, 16 aa, synthetic construct
  SITE 2: note = oxazolidine-4-carboxylic acid
  SITE 3: note = 4Hyp
  SITE 10: note = t-butyl glycine
CXXDAYLGCXSYCEPW

SEQ ID NO: 1205 - protein, 16 aa, synthetic construct
  SITE 2: note = cis-L-4-hydroxyproline
  SITE 3: note = 4Hyp
  SITE 10: note = t-butyl glycine
CXXDAYLGCXSYCEPW

SEQ ID NO: 1206 - protein, 16 aa, synthetic construct
  SITE 3: note = oxazolidine-4-carboxylic acid
  SITE 10: note = t-butyl glycine
CPXDAYLGCXSYCEPW

SEQ ID NO: 1207 - protein, 16 aa, synthetic construct
  SITE 3: note = cis-L-4-hydroxyproline
  SITE 10: note = t-butyl glycine
CPXDAYLGCXSYCEPW

SEQ ID NO: 1208 - protein, 16 aa, synthetic construct
  SITE 3: note = 4Hyp
  SITE 6: note = 3,4-dihydroxy-phenylalanine
  SITE 10: note = t-butyl glycine
CPXDAXLGCXSYCEPW

SEQ ID NO: 1209 - protein, 16 aa, synthetic construct
  SITE 3: note = 4Hyp
  SITE 6: note = L-4-carbamoylphenylalanine
  SITE 10: note = t-butyl glycine
CPXDAXLGCXSYCEPW

SEQ ID NO: 1210 - protein, 16 aa, synthetic construct
  SITE 3: note = 4Hyp
  SITE 6: note = 4-carboxy-L-phenylalanine
  SITE 10: note = t-butyl glycine
CPXDAXLGCXSYCEPW

SEQ ID NO: 1211 - protein, 16 aa, synthetic construct
  SITE 3: note = 4Hyp
  SITE 6: note = homo-tyrosine
  SITE 10: note = t-butyl glycine
CPXDAXLGCXSYCEPW

SEQ ID NO: 1212 - protein, 16 aa, synthetic construct
  SITE 3: note = 4Hyp
  SITE 8: note = D-Serine
  SITE 10: note = t-butyl glycine
CPXDAYLXCXSYCEPW

SEQ ID NO: 1213 - protein, 16 aa, synthetic construct
  SITE 3: note = 4Hyp
  SITE 8: note = D-Threonine
  SITE 10: note = t-butyl glycine
CPXDAYLXCXSYCEPW

SEQ ID NO: 1214 - protein, 16 aa, synthetic construct
  SITE 3: note = 4Hyp
  SITE 8: note = D-Aspartic Acid
  SITE 10: note = t-butyl glycine
CPXDAYLXCXSYCEPW

SEQ ID NO: 1215 - protein, 16 aa, synthetic construct
  SITE 3: note = 4Hyp
  SITE 8: note = D-glutamic acid
  SITE 10: note = t-butyl glycine
CPXDAYLXCXSYCEPW

SEQ ID NO: 1216 - protein, 16 aa, synthetic construct
  SITE 3: note = 4Hyp
  SITE 8: note = D-asparagine
  SITE 10: note = t-butyl glycine
CPXDAYLXCXSYCEPW

SEQ ID NO: 1217 - protein, 16 aa, synthetic construct
  SITE 3: note = 4Hyp
  SITE 8: note = D-Glutamine
  SITE 10: note = t-butyl glycine
CPXDAYLXCXSYCEPW

SEQ ID NO: 1218 - protein, 16 aa, synthetic construct
  SITE 3: note = 4Hyp
  SITE 8: note = D-Tyrosine
  SITE 10: note = t-butyl glycine
CPXDAYLXCXSYCEPW

SEQ ID NO: 1219 - protein, 16 aa, synthetic construct
  SITE 3: note = 4Hyp
  SITE 10: note = t-butyl glycine
CPXDAYLSCXSYCEPW

SEQ ID NO: 1220 - protein, 16 aa, synthetic construct
  SITE 3: note = 4Hyp
  SITE 10: note = t-butyl glycine
CPXDAYLDCXSYCEPW

SEQ ID NO: 1221 - protein, 16 aa, synthetic construct
  SITE 3: note = 4Hyp
  SITE 10: note = t-butyl glycine
CPXDAYLYCXSYCEPW

SEQ ID NO: 1222 - protein, 16 aa, synthetic construct
  SITE 3: note = 4Hyp
  SITE 10: note = t-butyl glycine
CPXDAYLNCXSYCEPW

SEQ ID NO: 1223 - protein, 16 aa, synthetic construct
  SITE 3: note = 4Hyp
  SITE 10: note = t-butyl glycine
  SITE 12: note = 3,4-dihydroxy-phenylalanine
CPXDAYLGCXSXCEPW

SEQ ID NO: 1224 - protein, 16 aa, synthetic construct
  SITE 3: note = 4Hyp
  SITE 10: note = t-butyl glycine
  SITE 12: note = L-4-carbamoylphenylalanine
CPXDAYLGCXSXCEPW

SEQ ID NO: 1225 - protein, 16 aa, synthetic construct
  SITE 3: note = 4Hyp
  SITE 10: note = t-butyl glycine
  SITE 12: note = 4-carboxy-L-phenylalanine
CPXDAYLGCXSXCEPW

SEQ ID NO: 1226 - protein, 16 aa, synthetic construct
  SITE 3: note = 4Hyp
  SITE 10: note = t-butyl glycine
  SITE 12: note = homo-tyrosine
CPXDAYLGCXSXCEPW

SEQ ID NO: 1227 - protein, 16 aa, synthetic construct
  SITE 3: note = 4Hyp
  SITE 15: note = 4Hyp
  SITE 10: note = t-butyl glycine
CPXDAYLGCXSYCEXW

SEQ ID NO: 1228 - protein, 16 aa, synthetic construct
  SITE 3: note = 4Hyp
  SITE 10: note = t-butyl glycine
  SITE 15: note = oxazolidine-4-carboxylic acid
CPXDAYLGCXSYCEXW

SEQ ID NO: 1229 - protein, 16 aa, synthetic construct
  SITE 3: note = 4Hyp
  SITE 10: note = t-butyl glycine
  SITE 15: note = cis-L-4-hydroxyproline
CPXDAYLGCXSYCEXW

SEQ ID NO: 1230 - protein, 16 aa, synthetic construct
  SITE 3: note = 4Hyp
  SITE 10: note = t-butyl glycine
CPXDAYLGCXSYCEPY

SEQ ID NO: 1231 - protein, 16 aa, synthetic construct
  SITE 3: note = 4Hyp
  SITE 10: note = t-butyl glycine
  SITE 16: note = 3,4-dihydroxy-phenylalanine
CPXDAYLGCXSYCEPX

SEQ ID NO: 1232 - protein, 16 aa, synthetic construct
  SITE 3: note = 4Hyp
  SITE 10: note = t-butyl glycine
  SITE 16: note = L-4-carbamoylphenylalanine
CPXDAYLGCXSYCEPX

SEQ ID NO: 1233 - protein, 16 aa, synthetic construct
  SITE 3: note = 4Hyp
  SITE 10: note = t-butyl glycine
  SITE 16: note = 4-carboxy-L-phenylalanine
CPXDAYLGCXSYCEPX

SEQ ID NO: 1234 - protein, 16 aa, synthetic construct
  SITE 3: note = 4Hyp
  SITE 10: note = t-butyl glycine
  SITE 16: note = homo-tyrosine
CPXDAYLGCXSYCEPX

SEQ ID NO: 1235 - protein, 16 aa, synthetic construct
  SITE 3: note = 4Hyp
  SITE 10: note = t-butyl glycine
CPXEAYLGCXSYCEPW

SEQ ID NO: 1236 - protein, 16 aa, synthetic construct
  SITE 3: note = 4Hyp
  SITE 4: note = L-gamma-carboxyglutamic acid
  SITE 10: note = t-butyl glycine
CPXXAYLGCXSYCEPW

SEQ ID NO: 1237 - protein, 16 aa, synthetic construct
  SITE 3: note = 4Hyp
  SITE 10: note = t-butyl glycine
CPXDAYSGCXSYCEPW

SEQ ID NO: 1238 - protein, 16 aa, synthetic construct
  SITE 3: note = 4Hyp
  SITE 10: note = t-butyl glycine
CPXDAYTGCXSYCEPW

SEQ ID NO: 1239 - protein, 16 aa, synthetic construct
  SITE 3: note = 4Hyp
  SITE 10: note = t-butyl glycine
CPXDAYDGCXSYCEPW

SEQ ID NO: 1240 - protein, 16 aa, synthetic construct
  SITE 3: note = 4Hyp
  SITE 10: note = t-butyl glycine
CPXDAYEGCXSYCEPW

SEQ ID NO: 1241 - protein, 16 aa, synthetic construct
  SITE 3: note = 4Hyp
  SITE 10: note = t-butyl glycine
CPXDAYNGCXSYCEPW

SEQ ID NO: 1242 - protein, 16 aa, synthetic construct
  SITE 3: note = 4Hyp
  SITE 10: note = t-butyl glycine
CPXDAYQGCXSYCEPW

SEQ ID NO: 1243 - protein, 16 aa, synthetic construct
  SITE 3: note = 4Hyp
  SITE 10: note = t-butyl glycine
  SITE 11: note = homoserine
CPXDAYLGCXXYCEPW

SEQ ID NO: 1244 - protein, 16 aa, synthetic construct
  SITE 3: note = 4Hyp
  SITE 10: note = t-butyl glycine
CPXDAYLGCXTYCEPW

SEQ ID NO: 1245 - protein, 16 aa, synthetic construct
  SITE 3: note = 4Hyp
  SITE 10: note = t-butyl glycine
CPXDAYLGCXDYCEPW

SEQ ID NO: 1246 - protein, 16 aa, synthetic construct
  SITE 3: note = 4Hyp
  SITE 10: note = t-butyl glycine
CPXDAYLGCXEYCEPW

SEQ ID NO: 1247 - protein, 16 aa, synthetic construct
  SITE 3: note = 4Hyp
  SITE 10: note = t-butyl glycine
CPXDAYLGCXNYCEPW

SEQ ID NO: 1248 - protein, 16 aa, synthetic construct
  SITE 3: note = 4Hyp
  SITE 10: note = t-butyl glycine
CPXDAYLGCXQYCEPW

SEQ ID NO: 1249 - protein, 16 aa, synthetic construct
  SITE 3: note = 4Hyp
  SITE 10: note = t-butyl glycine
CPXDAYLGCXSYCDPW

SEQ ID NO: 1250 - protein, 16 aa, synthetic construct
  SITE 3: note = 4Hyp
  SITE 10: note = t-butyl glycine
  SITE 14: note = L-gamma-carboxyglutamic acid
CPXDAYLGCXSYCXPW

SEQ ID NO: 1251 - protein, 16 aa, synthetic construct
  SITE 3: note = 4Hyp
CPXDAYLGCYSYCEPW

SEQ ID NO: 1252 - protein, 16 aa, synthetic construct
  SITE 3: note = 4Hyp
  SITE 10: note = 3-hydroxy-L-valine
CPXDAYLGCXSYCEPW

SEQ ID NO: 1253 - protein, 17 aa, synthetic construct
  SITE 3: note = 4Hyp
  SITE 10: note = t-butyl glycine
  SITE 17: note = 6-azido lysine
CPXDAYLGCXSYCEPWX

SEQ ID NO: 1254 - RNA, 23 nt, synthetic construct
  source 1..23: mol_type = other RNA
  modified_base 1: mod_base = OTHER; note = thymidine
tataaatagattctgtagcttaa

SEQ ID NO: 1255 - RNA, 23 nt, synthetic construct
  source 1..23: mol_type = other RNA
  modified_base 1: mod_base = OTHER; note = thymidine
  modified_base <1: mod_base = OTHER; note = vinyl phosphonate
  modified_base 1: mod_base = OTHER; note = 2'-O-methoxyethyl thymidine 3'-phosphorothioate
  modified_base 2: mod_base = OTHER; note = 2'-fluoro adenosine 3'-phosphorothioate
  modified_base 3: mod_base = OTHER; note = 2'-O-methyl uridine 3'-phosphodiester
  modified_base 4..6: mod_base = OTHER; note = 2'-O-methyl adenosine 3'-phosphodiester
  modified_base 7: mod_base = OTHER; note = 2'-O-methyl uridine 3'-phosphodiester
  modified_base 8: mod_base = OTHER; note = 2'-O-methyl adenosine 3'-phosphodiester
  modified_base 9: mod_base = OTHER; note = 2'-O-methyl guanosine 3'-phosphodiester
  modified_base 10: mod_base = OTHER; note = 2'-O-methyl adenosine 3'-phosphodiester
  modified_base 11..12: mod_base = OTHER; note = 2'-O-methyl uridine 3'-phosphodiester
  modified_base 13: mod_base = OTHER; note = 2'-O-methyl cytidine 3'-phosphodiester
  modified_base 14: mod_base = OTHER; note = 2'-fluoro uridine 3'-phosphodiester
  modified_base 15: mod_base = OTHER; note = 2'-O-methyl guanosine 3'-phosphodiester
  modified_base 16: mod_base = OTHER; note = 2'-fluoro uridine 3'-phosphodiester
  modified_base 17: mod_base = OTHER; note = 2'-O-methyl adenosine 3'-phosphodiester
  modified_base 18: mod_base = OTHER; note = 2'-O-methyl guanosine 3'-phosphodiester
  modified_base 19: mod_base = OTHER; note = 2'-O-methyl cytidine 3'-phosphodiester
  modified_base 20: mod_base = OTHER; note = 2'-O-methyl uridine 3'-phosphodiester
  modified_base 21: mod_base = OTHER; note = 2'-O-methyl uridine 3'-phosphorothioate
  modified_base 22: mod_base = OTHER; note = 2'-O-methoxyethyl adenosine 3'-phosphorothioate
  modified_base 23: mod_base = OTHER; note = 2'-O-methoxyethyl adenosine
tataaatagattctgtagcttaa

SEQ ID NO: 1256 - RNA, 21 nt, synthetic construct
  source 1..21: mol_type = other RNA
caatttctgtctcatcttaaa

SEQ ID NO: 1257 - RNA, 21 nt, synthetic construct
  source 1..21: mol_type = other RNA
aagctacagaatctatttata

SEQ ID NO: 1258 - RNA, 23 nt, synthetic construct
  source 1..23: mol_type = other RNA
  modified_base 23: mod_base = i
  modified_base 1: mod_base = OTHER; note = thymine
tttaagatgagacagaaattgan

SEQ ID NO: 1259 - RNA, 23 nt, synthetic construct
  source 1..23: mol_type = other RNA
  modified_base 22..23: mod_base = i
  modified_base 1: mod_base = OTHER; note = thymine
tttaagatgagacagaaattgnn

SEQ ID NO: 1260 - RNA, 23 nt, synthetic construct
  source 1..23: mol_type = other RNA
  modified_base 22: mod_base = i
  modified_base 1: mod_base = OTHER; note = thymine
tttaagatgagacagaaattgna

SEQ ID NO: 1261 - RNA, 23 nt, synthetic construct
  source 1..23: mol_type = other RNA
  modified_base 23: mod_base = i
  modified_base 1: mod_base = OTHER; note = thymine
tataaatagattctgtagcttan

SEQ ID NO: 1262 - RNA, 23 nt, synthetic construct
  source 1..23: mol_type = other RNA
  modified_base 22..23: mod_base = i
  modified_base 1: mod_base = OTHER; note = thymine
tataaatagattctgtagcttnn

SEQ ID NO: 1263 - RNA, 23 nt, synthetic construct
  source 1..23: mol_type = other RNA
  modified_base 22: mod_base = i
  modified_base 1: mod_base = OTHER; note = thymine
tataaatagattctgtagcttna

SEQ ID NO: 1264 - RNA, 23 nt, synthetic construct
  source 1..23: mol_type = other RNA
  modified_base 23: mod_base = i
  modified_base 1: mod_base = OTHER; note = thymine
  modified_base 7: mod_base = OTHER; note = thymine
tataaatagattctgtagcttan

SEQ ID NO: 1265 - RNA, 23 nt, synthetic construct
  source 1..23: mol_type = other RNA
  modified_base 22..23: mod_base = i
  modified_base 7: mod_base = OTHER; note = thymine
  modified_base 1: mod_base = OTHER; note = thymine
tataaatagattctgtagcttnn

SEQ ID NO: 1266 - RNA, 23 nt, synthetic construct
  source 1..23: mol_type = other RNA
  modified_base 22: mod_base = i
  modified_base 7: mod_base = OTHER; note = thymine
  modified_base 1: mod_base = OTHER; note = thymine
tataaatagattctgtagcttna

SEQ ID NO: 1267 - RNA, 23 nt, synthetic construct
  source 1..23: mol_type = other RNA
  modified_base 23: mod_base = i
  modified_base 6: mod_base = OTHER; note = thymine
  modified_base 1: mod_base = OTHER; note = thymine
ttaagttttagtcttaatcttan

SEQ ID NO: 1268 - RNA, 23 nt, synthetic construct
  source 1..23: mol_type = other RNA
  modified_base 22..23: mod_base = i
  modified_base 1: mod_base = OTHER; note = thymine
  modified_base 6: mod_base = OTHER; note = thymine
ttaagttttagtcttaatcttnn

SEQ ID NO: 1269 - RNA, 23 nt, synthetic construct
  source 1..23: mol_type = other RNA
  modified_base 22: mod_base = i
  modified_base 1: mod_base = OTHER; note = thymine
  modified_base 6: mod_base = OTHER; note = thymine
ttaagttttagtcttaatcttna

SEQ ID NO: 1270 - RNA, 23 nt, synthetic construct
  source 1..23: mol_type = other RNA
  modified_base 23: mod_base = i
  modified_base 1: mod_base = OTHER; note = thymine
  modified_base 7: mod_base = OTHER; note = thymine
ttaagttttagtcttaatcttan

SEQ ID NO: 1271 - RNA, 23 nt, synthetic construct
  source 1..23: mol_type = other RNA
  modified_base 22..23: mod_base = i
  modified_base 1: mod_base = OTHER; note = thymine
  modified_base 7: mod_base = OTHER; note = thymine
ttaagttttagtcttaatcttnn

SEQ ID NO: 1272 - RNA, 23 nt, synthetic construct
  source 1..23: mol_type = other RNA
  modified_base 22: mod_base = i
  modified_base 1: mod_base = OTHER; note = thymine
  modified_base 7: mod_base = OTHER; note = thymine
ttaagttttagtcttaatcttna

SEQ ID NO: 1273 - RNA, 22 nt, synthetic construct
  source 1..22: mol_type = other RNA
  modified_base 1: mod_base = OTHER; note = thymine
tttaagatgagacagaaattga

SEQ ID NO: 1274 - RNA, 22 nt, synthetic construct
  source 1..22: mol_type = other RNA
  modified_base 22: mod_base = i
  modified_base 1: mod_base = OTHER; note = thymine
tttaagatgagacagaaattgn

SEQ ID NO: 1275 - RNA, 21 nt, synthetic construct
  source 1..21: mol_type = other RNA
  modified_base 1: mod_base = OTHER; note = thymine
tttaagatgagacagaaattg

SEQ ID NO: 1276 - RNA, 22 nt, synthetic construct
  source 1..22: mol_type = other RNA
  modified_base 1: mod_base = OTHER; note = thymine
tataaatagattctgtagctta

SEQ ID NO: 1277 - RNA, 22 nt, synthetic construct
  source 1..22: mol_type = other RNA
  modified_base 22: mod_base = i
  modified_base 1: mod_base = OTHER; note = thymine
tataaatagattctgtagcttn

SEQ ID NO: 1278 - RNA, 21 nt, synthetic construct
  source 1..21: mol_type = other RNA
  modified_base 1: mod_base = OTHER; note = thymine
tataaatagattctgtagctt

SEQ ID NO: 1279 - RNA, 22 nt, synthetic construct
  source 1..22: mol_type = other RNA
  modified_base 1: mod_base = OTHER; note = thymine
  modified_base 7: mod_base = OTHER; note = thymine
tataaatagattctgtagctta

SEQ ID NO: 1280 - RNA, 22 nt, synthetic construct
  source 1..22: mol_type = other RNA
  modified_base 22: mod_base = i
  modified_base 1: mod_base = OTHER; note = thymine
  modified_base 7: mod_base = OTHER; note = thymine
tataaatagattctgtagcttn

SEQ ID NO: 1281 - RNA, 21 nt, synthetic construct
  source 1..21: mol_type = other RNA
  modified_base 1: mod_base = OTHER; note = thymine
  modified_base 7: mod_base = OTHER; note = thymine
tataaatagattctgtagctt

SEQ ID NO: 1282 - RNA, 22 nt, synthetic construct
  source 1..22: mol_type = other RNA
  modified_base 1: mod_base = OTHER; note = thymine
  modified_base 6: mod_base = OTHER; note = thymine
ttaagttttagtcttaatctta

SEQ ID NO: 1283 - RNA, 22 nt, synthetic construct
  source 1..22: mol_type = other RNA
  modified_base 22: mod_base = i
  modified_base 1: mod_base = OTHER; note = thymine
  modified_base 6: mod_base = OTHER; note = thymine
ttaagttttagtcttaatcttn

SEQ ID NO: 1284 - RNA, 21 nt, synthetic construct
  source 1..21: mol_type = other RNA
  modified_base 1: mod_base = OTHER; note = thymine
  modified_base 6: mod_base = OTHER; note = thymine
ttaagttttagtcttaatctt

SEQ ID NO: 1285 - RNA, 22 nt, synthetic construct
  source 1..22: mol_type = other RNA
  modified_base 1: mod_base = OTHER; note = thymine
  modified_base 7: mod_base = OTHER; note = thymine
ttaagttttagtcttaatctta

SEQ ID NO: 1286 - RNA, 22 nt, synthetic construct
  source 1..22: mol_type = other RNA
  modified_base 22: mod_base = i
  modified_base 1: mod_base = OTHER; note = thymine
  modified_base 7: mod_base = OTHER; note = thymine
ttaagttttagtcttaatcttn

SEQ ID NO: 1287 - RNA, 21 nt, synthetic construct
  source 1..21: mol_type = other RNA
  modified_base 1: mod_base = OTHER; note = thymine
  modified_base 7: mod_base = OTHER; note = thymine
ttaagttttagtcttaatctt

SEQ ID NO: 1288 - RNA, 21 nt, synthetic construct
  source 1..21: mol_type = other RNA
aagctacagaatctatttata

SEQ ID NO: 1289 - RNA, 21 nt, synthetic construct
  source 1..21: mol_type = other RNA
caatttctgtctcatcttaaa

SEQ ID NO: 1290 - RNA, 21 nt, synthetic construct
  source 1..21: mol_type = other RNA
  modified_base 20: mod_base = OTHER; note = thymine
aagctacagaatctatttata